(19) United States
(12) Patent Application Publication
(10) Pub. No.: US 2025/0127038 A1
(43) Pub. Date: Apr. 17, 2025

(54) LIGHT EMITTING ELEMENT, AMINE COMPOUND FOR THE SAME, AND DISPLAY DEVICE INCLUDING THE LIGHT EMITTING ELEMENT

(71) Applicant: Samsung Display Co., Ltd., Yongin-si (KR)

(72) Inventor: Naoya SAKAMOTO, Yokohama (JP)

(21) Appl. No.: 18/903,617

(22) Filed: Oct. 1, 2024

(30) Foreign Application Priority Data
Oct. 6, 2023 (KR) 10-2023-0133643

(51) Int. Cl. H10K 85/60; H10K 50/15; H10K 50/18

(52) U.S. Cl. CPC H10K 85/633; H10K 50/15; H10K 50/181; H10K 85/615; H10K 85/622; H10K 85/6572; H10K 85/6574; H10K 85/6576

(57) ABSTRACT

A light emitting element, an amine compound for the light emitting element, and a display device including the light emitting element are provided. The light emitting element includes a first electrode, a second electrode disposed on the first electrode, and at least one functional layer disposed between the first electrode and the second electrode, and including an amine compound represented by Formula 1, thereby improving lifetime and efficiency.

### CROSS-REFERENCE TO RELATED APPLICATIONS

This application claims priority to and the benefit of Korean Patent Application No. 10-2023-0133643, filed on Oct. 6, 2023, in the Korean Intellectual Property Office, the entire content of which is incorporated herein by reference.

### BACKGROUND

The present disclosure herein relates to a light emitting element, an amine compound for the light emitting element, and a display device including the light emitting element.

Recently, the development of an organic electroluminescence display device, e.g., as an image display device, is being actively conducted or pursued. The organic electroluminescence display device may be a so-called “self-luminescent”-type or kind of light emitting element in which holes and electrons injected from a first electrode and a second electrode, respectively, combine in an emission layer of the display device. Subsequently, a light emitting material, e.g., an organic compound, in the emission layer (e.g., light emitting layer) emits light to achieve display (e.g., of an image).

The application of a light emitting element to a display device, requires, or there is a desire for, an increase of emission efficiency and lifetime. For example, it is desired or required, to develop materials for a light emitting element, capable of stably achieving the above goals and/or requirements, and these goals and/or requirements are being consistently pursued.

### SUMMARY

One or more aspects of embodiments of the present disclosure are directed toward a light emitting element having improved emission efficiency and lifetime characteristics.

One or more aspects of embodiments of the present disclosure are directed toward an amine compound that may improve the emission efficiency and lifetime of a light emitting element.

One or more aspects of embodiments of the present disclosure are directed toward a display device including the light emitting element and having excellent or suitable display quality.

Additional aspects will be set forth in part in the description which follows and, in part, will be apparent from the description, or may be learned by practice of the presented embodiments of the disclosure.

A light emitting element according to one or more embodiments includes a first electrode, a second electrode arranged on the first electrode, and at least one functional layer arranged between the first electrode and the second electrode, and including an amine compound of one or more embodiments. The amine compound of one or more embodiments may be represented by Formula 1.

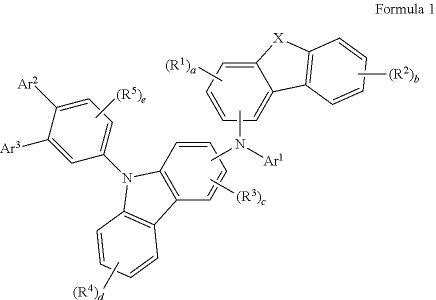

In Formula 1, X may be O or S, Ar¹ may be a substituted or unsubstituted aryl group of 6 to 30 ring-forming carbon atoms, or a substituted or unsubstituted heteroaryl group of 2 to 30 ring-forming carbon atoms. Ar² and Ar³ may each independently be a hydrogen atom, a deuterium atom, or a substituted or unsubstituted aryl group of 6 to 30 ring-forming carbon atoms, where at least one selected from among Ar² and Ar³ is a substituted or unsubstituted aryl group of 10 to 30 ring-forming carbon atoms. R¹ to R⁵ may each independently be a hydrogen atom, a deuterium atom, a halogen atom, a substituted or unsubstituted alkyl group of 1 to 15 carbon atoms, a substituted or unsubstituted aryl group of 6 to 30 ring-forming carbon atoms, or a substituted or unsubstituted heteroaryl group of 2 to 30 ring-forming carbon atoms. “a”, “c” and “e” may each independently be an integer of 0 to 3, and “b” and “d” may each independently be an integer of 0 to 4. In a case of Formula 1 where (e.g., when) the nitrogen atom of the amine compound is connected with position 2 or position 6 of a carbazole moiety, while being connected with position 3 or position 6 of a dibenzothiophene moiety or a dibenzofuran moiety, and/or in a case of Formula 1 where the nitrogen atom of the amine compound is connected with position 4 or position 5 of a carbazole moiety, while being connected with position 3 or position 6 of a dibenzothiophene moiety or a dibenzofuran moiety, a+b≥1 may be satisfied, and at least one selected from among R¹ and R² may not be a hydrogen atom. In a case of Formula 1 where (e.g., when) the nitrogen atom of the amine compound is connected with position 3 of the carbazole moiety, R³ may be a hydrogen atom or a deuterium atom. A case of Formula 1 where Ar¹ comprises a moiety of

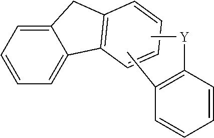

(where Y is O or S), may be excluded.

In one or more embodiments, the at least one functional layer may include an emission layer, a hole transport region arranged between the first electrode and the emission layer, and an electron transport region arranged between the emission layer and the second electrode, and the hole transport region may include the amine compound.

In one or more embodiments, the hole transport region may include at least one selected from among a hole injection layer, a hole transport layer and an electron blocking layer, and at least one selected from among the hole injection layer, the hole transport layer and the electron blocking layer may include the amine compound.

In one or more embodiments, the hole transport region may include a hole injection layer arranged on the first electrode, and a hole transport layer arranged on the hole injection layer, and the hole transport layer may include the amine compound.

In one or more embodiments, the amine compound represented by Formula 1 may be a monoamine compound.

In one or more embodiments, the amine compound represented by Formula 1 may be represented by Formula 2-1 or Formula 2-2.

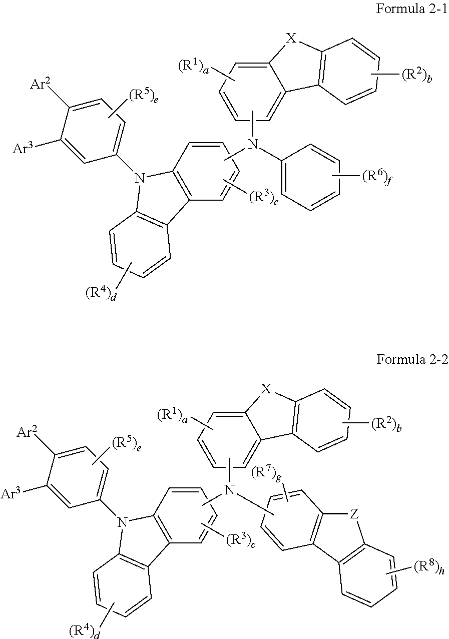

In Formula 2-1, R⁶ may be a hydrogen atom, a deuterium atom, a substituted or unsubstituted aryl group of 6 to 30 ring-forming carbon atoms, or a substituted or unsubstituted heteroaryl group of 2 to 30 ring-forming carbon atoms, and “f” is an integer of 0 to 5.

In Formula 2-2, Z may be O or S, R⁷ and R⁸ may each independently be a hydrogen atom or a deuterium atom, “g” may be an integer of 0 to 3, and “h” may be an integer of 0 to 4.

In Formula 2-1 and Formula 2-2, X, Ar², Ar³, R¹ to R⁵, and “a” to “e” may each independently be as defined in Formula 1.

In one or more embodiments, at least one selected from among Ar² and Ar³ may be a substituted or unsubstituted naphthyl group, a substituted or unsubstituted phenanthrenyl group, a substituted or unsubstituted triphenylenyl group, or a substituted or unsubstituted pyrenyl group.

In one or more embodiments, the amine compound represented by Formula 1 may be represented by Formula 3-1 or Formula 3-2.

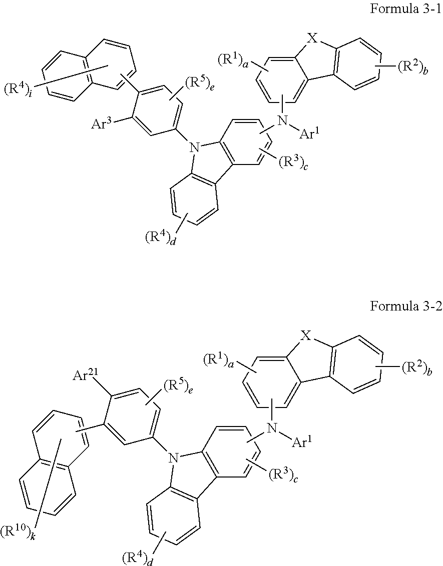

In Formula 3-1 and Formula 3-2, R⁹, R¹⁰, Ar²¹ and Ar³¹ may each independently be a hydrogen atom, a deuterium atom, or a substituted or unsubstituted aryl group of 6 to 30 ring-forming carbon atoms, “i” and “k” may each independently be an integer of 0 to 7, and X, Ar¹, R¹ to R⁵, and “a” to “e” may each independently be the same as defined in Formula 1.

In one or more embodiments, R¹ to R⁵ may each independently be a hydrogen atom, a deuterium atom, a halogen atom, a substituted or unsubstituted methyl group, or a substituted or unsubstituted phenyl group.

In one or more embodiments, the amine compound represented by Formula may be represented by one selected from among Formula 4-1 to Formula 4-4.

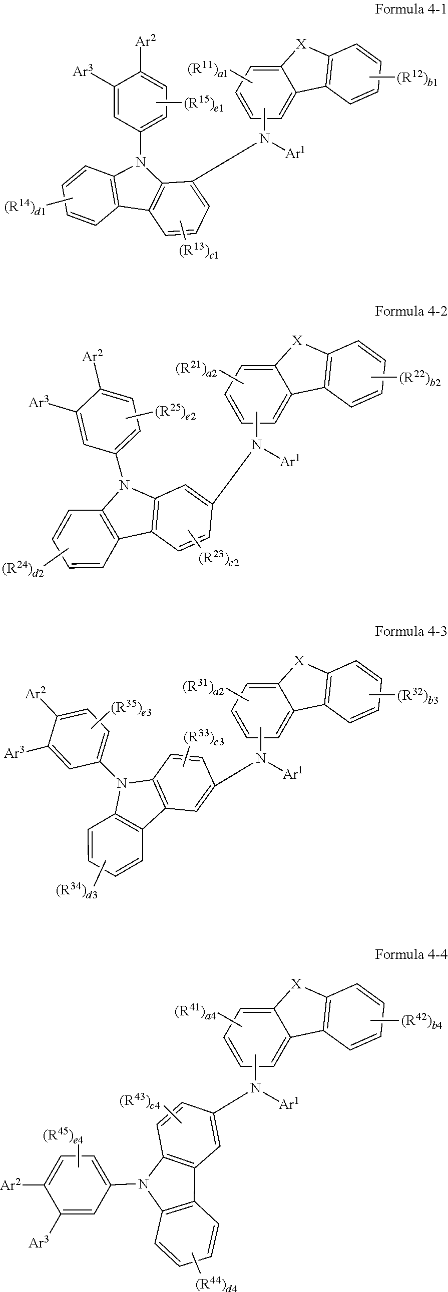

In Formula 4-1 to Formula 4-4, R¹¹ to R¹³, R²¹ to R²³, R³¹ to R³³, R⁴¹, and R⁴² may each independently be a hydrogen atom, a deuterium atom, or a substituted or unsubstituted aryl group of 6 to 30 ring-forming carbon atoms. R¹⁴, R²⁴, R³⁴, and R⁴⁴ may each independently be a hydrogen atom, a deuterium atom, a halogen atom, a substituted or unsubstituted alkyl group of 1 to 15 carbon atoms, or a substituted or unsubstituted aryl group of 6 to 30 ring-forming carbon atoms, R¹⁵, R²⁵, R³⁵, R⁴³ and R⁴⁵ may each independently be a hydrogen atom or a deuterium atom. a1 to a4, c1 to c4, and e1 to e4 may each independently be an integer of 0 to 3, b1 to b4, and d1 to d4 may each independently be an integer of 0 to 4. X, and Ar¹ to Ar³ may each independently be as defined in Formula 1.

In a case of Formula 4-2 where the nitrogen atom of the amine compound is connected with position 3 or position 6 of a dibenzothiophene moiety or a dibenzofuran moiety, a2+b2≥1 may be satisfied, and at least one selected from among R²¹ and R²² may be a substituted or unsubstituted aryl group of 6 to 30 ring-forming carbon atoms.

In some embodiments, in a case of Formula 4-4 where the nitrogen atom of the amine compound is connected with position 3 or position 6 of a dibenzothiophene moiety or a dibenzofuran moiety, a4+b4≥1 may be satisfied, and at least one selected from among R⁴¹ and R⁴² may be a substituted or unsubstituted aryl group of 6 to 30 ring-forming carbon atoms.

In one or more embodiments, at least one selected from among Ar² and Ar³ may be represented by one selected from among substituents in Substituent Group 1.

In one or more embodiments, the amine compound may be at least one selected from among compounds in Compound Group 1.

A display device of one or more embodiments includes a base layer, a circuit layer arranged on the base layer, and a display element layer arranged on the circuit layer and including a light emitting element. Here, the light emitting element includes a first electrode, a hole transport region arranged on the first electrode, an emission layer arranged on the hole transport region, an electron transport region arranged on the emission layer, and a second electrode arranged on the electron transport region, and the hole transport region includes an amine compound represented by Formula 1.

### BRIEF DESCRIPTION OF THE DRAWINGS

The accompanying drawings are included to provide a further understanding of the present disclosure and are incorporated in and constitute a part of this specification. The drawings illustrate example embodiments of the present disclosure and, together with the description, serve to explain principles of the present disclosure. In the drawings:

FIG. 1 is a plan view showing a display device according to one or more embodiments of the present disclosure;

FIG. 2 is a cross-sectional view of a display device according to one or more embodiments of the present disclosure;

FIG. 3 is a cross-sectional view schematically showing a light emitting element according to one or more embodiments of the present disclosure;

FIG. 4 is a cross-sectional view schematically showing a light emitting element according to one or more embodiments of the present disclosure;

FIG. 5 is a cross-sectional view schematically showing a light emitting element according to one or more embodiments of the present disclosure;

FIG. 6 is a cross-sectional view schematically showing a light emitting element according to one or more embodiments of the present disclosure;

FIG. 7 is a cross-sectional view of a display device according to one or more embodiments of the present disclosure;

FIG. 8 is a cross-sectional view of a display device according to one or more embodiments of the present disclosure;

FIG. 9 is a cross-sectional view showing a display device according to one or more embodiments of the present disclosure;

FIG. 10 is a cross-sectional view showing a display device according to one or more embodiments of the present disclosure; and

FIG. 11 is a view showing a vehicle in which a display device according to one or more embodiments of the present disclosure is arranged.

### DETAILED DESCRIPTION

The present disclosure may be modified in one or more suitable manners and have many forms, and thus specific embodiments will be exemplified in the drawings and described in more detail in the detailed description of present disclosure. It should be understood, however, that it is not intended to limit the present disclosure to the particular forms disclosed, but rather, is intended to cover all modifications, equivalents, and alternatives falling within the spirit and scope of the present disclosure.

When explaining each of drawings, like reference numbers are utilized for referring to like elements. In the accompanying drawings, the dimensions of each structure are exaggeratingly illustrated for clarity of the present disclosure. It will be understood that, although the terms “first,” “second,” and/or the like, may be utilized herein to describe one or more suitable components, these components should not be limited by these terms. These terms are only utilized to distinguish one component from another. For example, a first component could be termed a second component, and, similarly, a second component could be termed a first component, without departing from the scope of example embodiments of the present disclosure. As utilized herein, the singular forms, “a,” “an,” and “the” are intended to include the plural forms as well, unless the context clearly indicates otherwise.

In the present application, it will be understood that the terms “include,” “includes,” “including,” “comprise,” “comprises”, “comprising,” “has,” “having,” “have” and/or the like specify the presence of features, numbers, steps, operations, component, parts, and/or combinations thereof disclosed in the specification, but do not exclude the possibility of presence or addition of one or more other features, numbers, steps, operations, component, parts, or combinations thereof.

In the present application, when a layer, a film, a region, or a plate is referred to as being “on” or “in an upper portion of” another layer, film, region, or plate, it may be not only “directly on” the layer, film, region, or plate, but intervening layers, films, regions, or plates may also be present. On the contrary to this, when a layer, a film, a region, or a plate is referred to as being “below”, “in a lower portion of” another layer, film, region, or plate, it can be not only directly under the layer, film, region, or plate, but intervening layers, films, regions, or plates may also be present. In some embodiments, it will be understood that when a part is referred to as being “on” another part, it can be arranged above the other part, or arranged under the other part as well.

For example, the terms, such as “lower”, “above”, “upper” and/or the like, are utilized herein for ease of description to describe one element's relation to another element(s) as illustrated in the drawings. The terms are relative concepts and are described based on the directions indicated in the drawings. It will be understood that the terms have a relative concept and are described on the basis of the orientation depicted in the drawings. It will be understood that the spatially relative terms are intended to encompass different orientations of the device in use or operation in addition to the orientation depicted in the drawings. For example, if the device in the drawings is turned over, elements described as “beneath” other elements or features would then be oriented “above” or “over” the other elements or features. Thus, the term “beneath” may encompass both an orientation of above and below. The device may be otherwise oriented (rotated 90 degrees or at other orientations), and the spatially relative descriptors used herein should be interpreted accordingly.

As utilized herein, the terms “use,” “using,” and “used” may be considered synonymous with the terms “utilize,” “utilizing,” and “utilized,” respectively.

As utilized herein, expressions such as “at least one of,” “one of,” “selected from,” and “selected from among,” when preceding a list of elements, modify the entire list of elements and do not modify the individual elements of the list. For example, the expressions “at least one of a to c,” “at least one of a, b or c,” and “at least one of a, b and/or c” may indicate only a, only b, only c, both (e.g., simultaneously) a and b, both (e.g., simultaneously) a and c, both (e.g., simultaneously) b and c, all of a, b, and c, or variations thereof.

The term “and/or” includes all combinations of one or more of the associated listed elements.

As utilized herein, the term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

Unless otherwise defined, all terms (including chemical, technical and scientific terms) utilized herein have the same meaning as commonly understood by one of ordinary skill in the art to which this present disclosure belongs. It will be further understood that terms, such as those defined in commonly utilized dictionaries, should be interpreted as having a meaning that is consistent with their meaning in the context of the relevant art and will not be interpreted in an idealized or overly formal sense unless expressly so defined herein.

As utilized herein, the phrase “consisting essentially of” refers to that any additional components will not materially affect the chemical, physical, optical, or electrical properties of the semiconductor film.

As utilized herein, the phrase “on a plane,” or “plan view,” refers to viewing a target portion from the top, and the phrase “on a cross-section” refers to viewing a cross-section formed by vertically cutting a target portion from the side.

In present disclosure, “not include a or any ‘component’” “exclude a or any ‘component’”, “‘component’-free”, and/or the like refers to that the “component” not being added, selected or utilized as a component in the composition, but the “component” of less than a suitable amount may still be included due to other impurities and/or external factor.

### Definitions

In the specification, the term “substituted or unsubstituted” may refer to substituted or unsubstituted with at least one substituent selected from among the group including (e.g., consisting of) a deuterium atom, a halogen atom, a cyano group, a nitro group, an amino group, an amine group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In some embodiments, each of the substituents exemplified herein may be substituted or unsubstituted. For example, a biphenyl group may be interpreted as an aryl group or a phenyl group substituted with a phenyl group.

In the specification, the phrase “bonded to an adjacent group to form a ring” may refer to that a group is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In some embodiments, the rings formed by being bonded to each other may be connected to another ring to form a spiro structure.

In the specification, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In some embodiments, two methyl groups in 4,5-dimethylphenanthrene may be interpreted as “adjacent groups” to each other.

In the specification, examples of the halogen atom may include a fluorine atom, a chlorine atom, a bromine atom, or an iodine atom.

In the specification, the alkyl group may be linear or branched. The number of carbons in the alkyl group is 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and/or the like, but one or more embodiments of the present disclosure is not limited thereto.

In the specification, a cycloalkyl group may refer to a cyclic alkyl group. The number of carbons in the cycloalkyl group is 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, and/or the like, but one or more embodiments of the present disclosure is not limited thereto.

In the specification, an alkenyl group refers to a hydrocarbon group including at least one carbon double bond in the middle or terminal of an alkyl group having 2 or more carbon atoms. The alkenyl group may be linear or branched. The number of carbon atoms in the alkenyl group is not specifically limited, but is 2 to 30, 2 to 20, or 2 to 10. Examples of the alkenyl group include a vinyl group, a 1-butenyl group, a 1-pentenyl group, a 1,3-butadienyl group, a styrenyl group, a styryl vinyl group, and/or the like, but one or more embodiments of the present disclosure is not limited thereto.

In the specification, an alkynyl group refers to a hydrocarbon group including at least one carbon triple bond in the middle or terminal of an alkyl group having 2 or more carbon atoms. The alkynyl group may be linear or branched. Although the number of carbon atoms is not specifically limited, it is 2 to 30, 2 to 20, or 2 to 10. Specific examples of the alkynyl group may include an ethynyl group, a propynyl group, and/or the like, but are not limited thereto.

In the specification, the hydrocarbon ring group refers to any functional group or substituent derived from an aliphatic hydrocarbon ring. The hydrocarbon ring group may be a saturated hydrocarbon ring group having 5 to 20 ring-forming carbon atoms.

In the specification, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, and/or the like, but one or more embodiments of the present disclosure is not limited thereto.

In the specification, the fluorenyl group may be substituted, and two substituents may be bonded to each other to form a spiro structure. Examples of the substituted fluorenyl group are as follows. However, one or more embodiments of the present disclosure is not limited thereto.

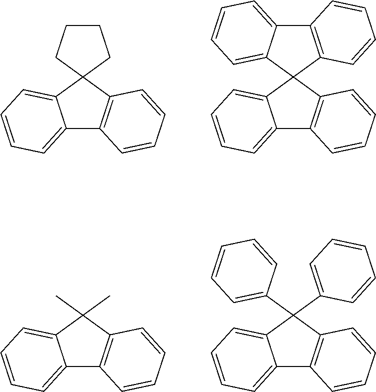

The heterocyclic group herein refers to any functional group or substituent derived from a ring containing at least one of B, O, N, P, Si, Se, or S as a heteroatom. The heterocyclic group includes an aliphatic heterocyclic group and an aromatic heterocyclic group. The aromatic heterocyclic group may be a heteroaryl group. The aliphatic heterocycle and the aromatic heterocycle may be monocyclic or polycyclic.

In the specification, the heterocyclic group may contain at least one of B, O, N, P, Si, Se, or S as a heteroatom. If the heterocyclic group contains two or more heteroatoms, the two or more heteroatoms may be the same as or different from each other. The heterocyclic group may be a monocyclic heterocyclic group or a polycyclic heterocyclic group, and includes a heteroaryl group. The number of ring-forming carbon atoms in the heterocyclic group may be 2 to 30, 2 to 20, or 2 to 10.

In the specification, the aliphatic heterocyclic group may include at least one of B, O, N, P, Si, Se, or S as a heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, and/or the like, but one or more embodiments of the present disclosure is not limited thereto.

In the specification, the heteroaryl group may contain at least one of B, O, N, P, Si, Se, or S as a heteroatom. If the heteroaryl group contains two or more heteroatoms, the two or more heteroatoms may be the same as or different from each other. The heteroaryl group may be a monocyclic heterocyclic group or a polycyclic heterocyclic group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, and/or the like, but one or more embodiments of the present disclosure is not limited thereto.

In the specification, the preceding description of the aryl group may be applied to an arylene group except that the arylene group is a divalent group. The preceding description of the heteroaryl group may be applied to a heteroarylene group except that the heteroarylene group is a divalent group.

In the specification, the silyl group includes an alkylsilyl group and an arylsilyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and/or the like, but one or more embodiments of the present disclosure is not limited thereto.

In the specification, the number of carbon atoms in the carbonyl group is not specifically limited, but may be 1 to 40, 1 to 30, or 1 to 20. For example, the carbonyl group may have the following structures, but one or more embodiments of the present disclosure is not limited thereto.

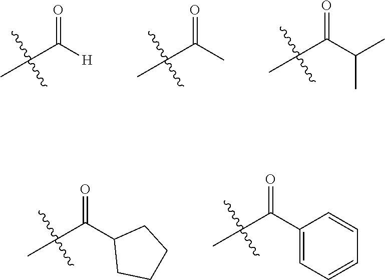

In the specification, the number of carbon atoms in the sulfinyl group and the sulfonyl group is not particularly limited, but may be 1 to 30. The sulfinyl group may include an alkyl sulfinyl group and an aryl sulfinyl group. The sulfonyl group may include an alkyl sulfonyl group and an aryl sulfonyl group.

In the specification, the thio group may include an alkylthio group and an arylthio group. The thio group may refer to that a sulfur atom is bonded to the alkyl group or the aryl group as defined herein. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but one or more embodiments of the present disclosure is not limited thereto.

In the specification, an oxy group may refer to that an oxygen atom is bonded to the alkyl group or the aryl group as defined herein. The oxy group may include an alkoxy group and an aryl oxy group. The alkoxy group may be a linear chain, a branched chain or a ring chain. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, and/or the like, but one or more embodiments of the present disclosure is not limited thereto.

The boron group herein may refer to that a boron atom is bonded to the alkyl group or the aryl group as defined herein. The boron group includes an alkyl boron group and an aryl boron group. Examples of the boron group may include a dimethylboron group, a t-butyldimethylboron group, a diphenylboron group, a phenylboron group, and/or the like, but one or more embodiments of the present disclosure is not limited thereto.

In the specification, the number of carbon atoms in an amine group is not specifically limited, but may be 1 to 30. The amine group may include an alkyl amine group and an aryl amine group. The alkyl group in the alkyl amine group may be a linear chain group, a branched chain group, or a cyclic group. The number of carbon atoms in the alkyl amine group is not specifically limited, but may be, for example, 1 to 20 or 1 to 10. The number of carbon atoms in the aryl amine group is not specifically limited, but may be, for example, 6 to 30, 6 to 20, or 6 to 15. Examples of the amine group may include a methylamine group, a dimethylamine group, a phenylamine group, a diphenylamine group, a naphthylamine group, a 9-methyl-anthracenylamine group, and/or the like, but one or more embodiments of the present disclosure is not limited thereto.

In the specification, the alkyl group among an alkylthio group, an alkylsulfoxy group, an alkylaryl group, an alkylamino group, an alkyl boron group, an alkyl silyl group, and an alkyl amine group is the same as the examples of the alkyl group described herein.

In the specification, the aryl group among an aryloxy group, an arylthio group, an arylsulfoxy group, an arylamino group, an arylboron group, an arylsilyl group, an arylamine group is the same as the examples of the aryl group described herein.

In the specification, a direct linkage may refer to a single bond.

In one or more embodiments, in the specification,

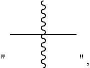

“ [symbol] ” “and”

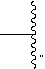

refer to a position to be connected.

Hereinafter, embodiments of the present disclosure will be described with reference to the accompanying drawings.

### Display Device

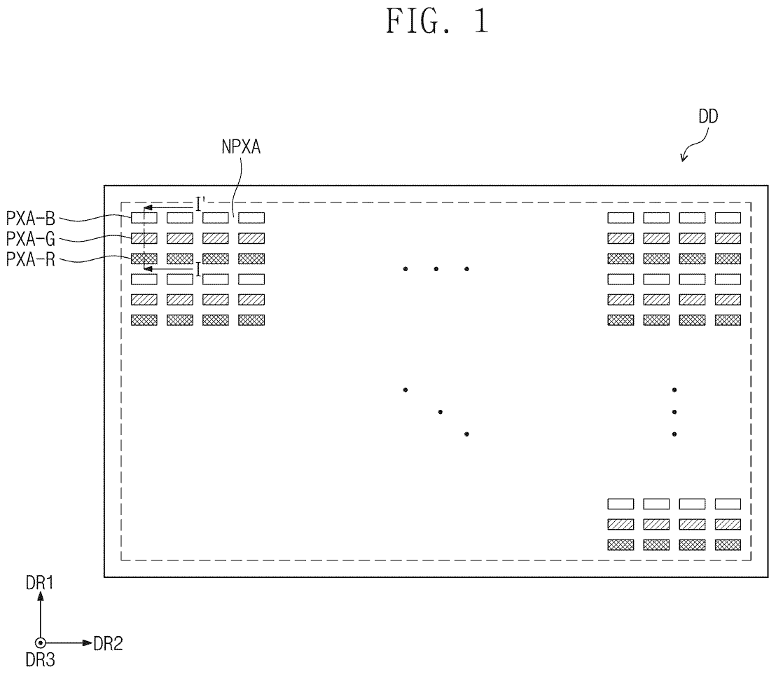

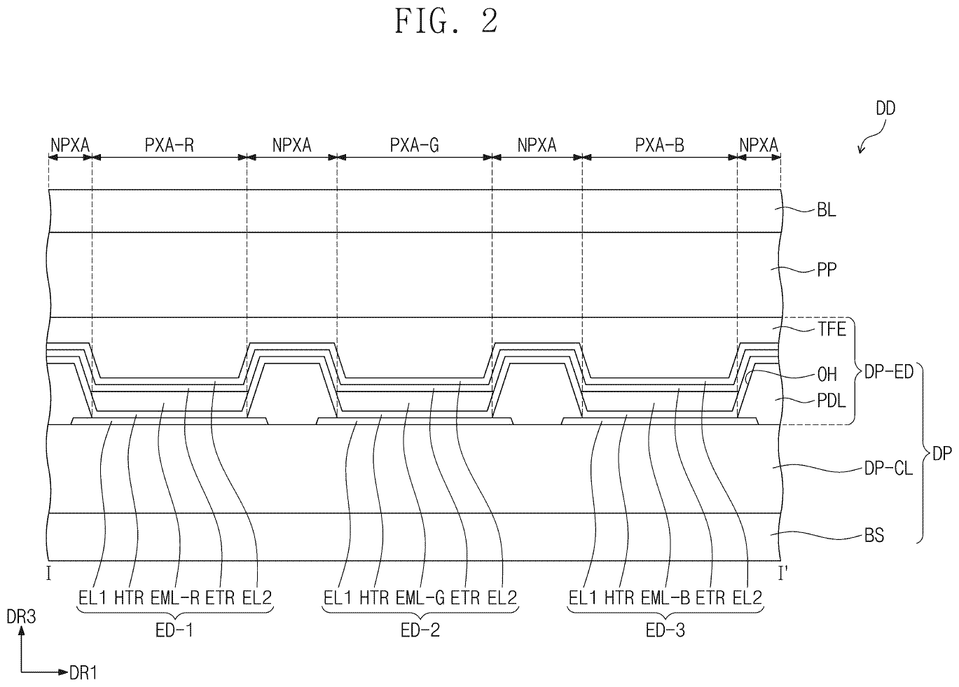

FIG. 1 is a plan view illustrating one or more embodiments of a display device DD. FIG. 2 is a cross-sectional view of the display device DD of the embodiment. FIG. 2 is a cross-sectional view illustrating a part taken along the line I-I′ of FIG. 1.

The display device DD may include a display panel DP and an optical layer PP arranged on the display panel DP. The display panel DP includes light emitting elements ED-1, ED-2, and ED-3. The display device DD may include a plurality of light emitting elements ED-1, ED-2, and ED-3. The optical layer PP may be arranged on the display panel DP to control reflected light in the display panel DP due to external light. The optical layer PP may include, for example, a polarization layer or a color filter layer. In one or more embodiments, unlike the configuration illustrated in the drawing, the optical layer PP may not be provided from the display device DD of one or more embodiments.

A base substrate BL may be arranged on the optical layer PP. The base substrate BL may be a member which provides a base surface on which the optical layer PP arranged. The base substrate BL may be a glass substrate, a metal substrate, a plastic substrate, and/or the like. However, one or more embodiments of the present disclosure is not limited thereto, and the base substrate BL may be an inorganic layer, an organic layer, or a composite material layer. In some embodiments, unlike the configuration illustrated, in one or more embodiments, the base substrate BL may not be provided.

The display device DD according to one or more embodiments may further include a filling layer. The filling layer may be arranged between a display device layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and the display device layer DP-ED. The display device layer DP-ED may include a pixel defining film PDL, the light emitting elements ED-1, ED-2, and ED-3 arranged between portions of the pixel defining film PDL, and an encapsulation layer TFE arranged on the light emitting elements ED-1, ED-2, and ED-3.

The base layer BS may be a member which provides a base surface on which the display device layer DP-ED is arranged. The base layer BS may be a glass substrate, a metal substrate, a plastic substrate, and/or the like. However, one or more embodiments is not limited thereto, and the base layer BS may be an inorganic layer, an organic layer, or a composite material layer.

In one or more embodiments, the circuit layer DP-CL is arranged on the base layer BS, and the circuit layer DP-CL may include a plurality of transistors. Each of the transistors may include a control electrode, an input electrode, and an output electrode. For example, the circuit layer DP-CL may include a switching transistor and a driving transistor for driving the light emitting elements ED-1, ED-2, and ED-3 of the display device layer DP-ED.

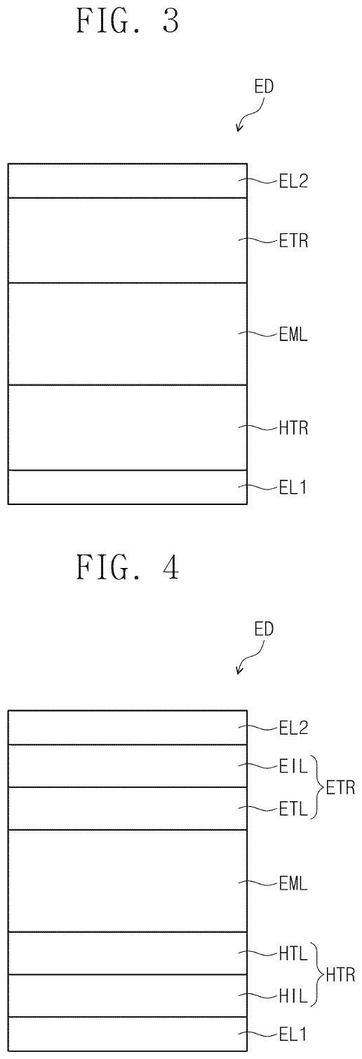

Each of the light emitting elements ED-1, ED-2, and ED-3 may have a structure of each light emitting element ED of embodiments according to FIGS. 3 to 6, as described in more detail elsewhere herein. Each of the light emitting elements ED-1, ED-2, and ED-3 may include a first electrode EL1, a hole transport region HTR, emission layers EML-R, EML-G, and EML-B, an electron transport region ETR, and a second electrode EL2.

FIG. 2 illustrates one or more embodiments in which the emission layers EML-R, EML-G, and EML-B of the light emitting elements ED-1, ED-2, and ED-3 are arranged in openings OH defined in the pixel defining film PDL, and the hole transport region HTR, the electron transport region ETR, and the second electrode EL2 are provided as a common layer in the entire light emitting elements ED-1, ED-2, and ED-3. However, one or more embodiments of the present disclosure is not limited thereto, and unlike the configuration illustrated in FIG. 2, the hole transport region HTR and the electron transport region ETR in one or more embodiments may be provided by being patterned inside the openings OH defined in the pixel defining film PDL. For example, the hole transport region HTR, the emission layers EML-R, EML-G, and EML-B, and the electron transport region ETR of the light emitting elements ED-1, ED-2, and ED-3 in one or more embodiments may be provided by being patterned in an inkjet printing method.

The encapsulation layer TFE may cover the light emitting elements ED-1, ED-2 and ED-3. The encapsulation layer TFE may seal the light emitting elements ED-1, ED-2 and ED-3 in the display device layer DP-ED. The encapsulation layer TFE may be a thin film encapsulation layer. The encapsulation layer TFE may be formed by laminating one layer or a plurality of layers. The encapsulation layer TFE includes at least one insulation layer. The encapsulation layer TFE according to one or more embodiments may include at least one inorganic film (hereinafter, an encapsulation-inorganic film). The encapsulation layer TFE according to one or more embodiments may also include at least one organic film (hereinafter, an encapsulation-organic film) and at least one encapsulation-inorganic film.

The encapsulation-inorganic film protects the display device layer DP-ED from moisture and/or oxygen, and the encapsulation-organic film protects the display device layer DP-ED from foreign substances such as dust particles. The encapsulation-inorganic film may include silicon nitride, silicon oxynitride, silicon oxide, titanium oxide, aluminum oxide, and/or the like, but one or more embodiments of the present disclosure is not particularly limited thereto. The encapsulation-organic film may include an acrylic-based compound, an epoxy-based compound, and/or the like. The encapsulation-organic film may include a photopolymerizable organic material, but one or more embodiments of the present disclosure is not particularly limited thereto.

The encapsulation layer TFE may be arranged on the second electrode EL2 and may be arranged filling the opening OH.

Referring to FIGS. 1 and 2, the display device DD may include one or more non-light emitting region(s) NPXA and also include light emitting regions PXA-R, PXA-G, and PXA-B. The light emitting regions PXA-R, PXA-G, and PXA-B may be regions in which light generated by the respective light emitting elements ED-1, ED-2, and ED-3 are emitted. The light emitting regions PXA-R, PXA-G, and PXA-B may be spaced and/or apart (e.g., spaced apart or separated) from each other on a plane (e.g., in a plan view).

Each of the light emitting regions PXA-R, PXA-G, and PXA-B may be a region divided (i.e., defined) by the pixel defining film PDL. The non-light emitting regions NPXA may be areas between the adjacent light emitting regions PXA-R, PXA-G, and PXA-B, which correspond to the pixel defining film PDL. In one or more embodiments, in the specification, the light emitting regions PXA-R, PXA-G, and PXA-B may respectively correspond to pixels. The pixel defining film PDL may divide the light emitting elements ED-1, ED-2, and ED-3. The emission layers EML-R, EML-G, and EML-B of the light emitting elements ED-1, ED-2, and ED-3 may be arranged in openings OH defined in the pixel defining film PDL and separated from each other.

The light emitting regions PXA-R, PXA-G, and PXA-B may be divided into a plurality of groups according to the color of light generated from the light emitting elements ED-1, ED-2, and ED-3. In the display device DD of one or more embodiments illustrated in FIGS. 1 and 2, three light emitting regions PXA-R, PXA-G, and PXA-B, which emit red light, green light, and blue light, respectively, are exemplarily illustrated. For example, the display device DD of one or more embodiments may include the red light emitting region PXA-R, the green light emitting region PXA-G, and the blue light emitting region PXA-B that are separated from each other.

In the display device DD according to one or more embodiments, the plurality of light emitting elements ED-1, ED-2 and ED-3 may be to emit light beams having wavelengths different from each other. For example, in one or more embodiments, the display device DD may include a first light emitting element ED-1 that emits red light, a second light emitting element ED-2 that emits green light, and a third light emitting element ED-3 that emits blue light. For example, the red light emitting region PXA-R, the green light emitting region PXA-G, and the blue light emitting region PXA-B of the display device DD may correspond to the first light emitting element ED-1, the second light emitting element ED-2, and the third light emitting element ED-3, respectively.

However, one or more embodiments of the present disclosure is not limited thereto, and the first to third light emitting elements ED-1, ED-2, and ED-3 may be to emit light beams in substantially the same wavelength range or at least one light emitting element may be to emit a light beam in a wavelength range different from the others. For example, the first to third light emitting elements ED-1, ED-2, and ED-3 may all emit blue light.

The light emitting regions PXA-R, PXA-G, and PXA-B in the display device DD according to one or more embodiments may be arranged in a stripe form. Referring to FIG. 1, the plurality of red light emitting regions PXA-R may be arranged with each other along a second directional axis DR2, the plurality of green light emitting regions PXA-G may be arranged with each other along the second directional axis DR2, and the plurality of blue light emitting regions PXA-B each may be arranged along the second directional axis DR2. In some embodiments, the red light emitting region PXA-R, the green light emitting region PXA-G, and the blue light emitting region PXA-B may be alternately arranged in this order along a first directional axis DR1.

FIGS. 1 and 2 illustrate that all the light emitting regions PXA-R, PXA-G, and PXA-B have similar area, but one or more embodiments of the present disclosure is not limited thereto. Thus, the light emitting regions PXA-R, PXA-G, and PXA-B may have different areas from each other according to the wavelength range of the emitted light. In this case, the areas of the light emitting regions PXA-R, PXA-G, and PXA-B may refer to areas when viewed on a plane defined by the first directional axis DR1 and the second directional axis DR2. A third directional axis DR3 may be perpendicular to a plane defined by the first directional axis DR1 and the second directional axis DR2.

In one or more embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality desired or required in the display device DD. For example, the arrangement form of the light emitting regions PXA-R, PXA-G, and PXA-B may be a pentile (PENTILE™) arrangement form or a diamond (Diamond Pixel™) arrangement form, (PENTILE™ and Diamond Pixel™ are registered trademarks owned by Samsung Display Co., Ltd.).

In some embodiments, the areas of the light emitting regions PXA-R, PXA-G, and PXA-B may be different from each other. For example, in one or more embodiments, the area of the green light emitting region PXA-G may be smaller than that of the blue light emitting region PXA-B, but one or more embodiments of the present disclosure is not limited thereto.

Hereinafter, FIG. 3 to FIG. 6 are cross-sectional views schematically showing light emitting elements according to one or more embodiments. The light emitting element ED according to one or more embodiments may include a first electrode EL1, a second electrode EL2 oppositely arranged to the first electrode EL1, and at least one functional layer arranged between the first electrode EL1 and the second electrode EL2. The light emitting element ED of one or more embodiments may include an amine compound of one or more embodiments, which will be explained later, in the at least one functional layer.

The light emitting element ED may include a hole transport region HTR, an emission layer EML, an electron transport region ETR and/or the like, stacked in order as the at least one functional layer. Referring to FIG. 3, the light emitting element ED of one or more embodiments may include a first electrode EL1, a hole transport region HTR, an emission layer EML, an electron transport region ETR, and a second electrode EL2 stacked in order.

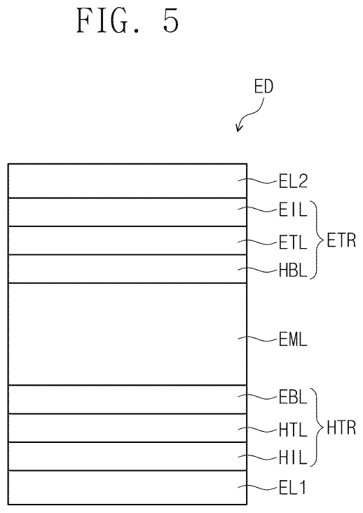

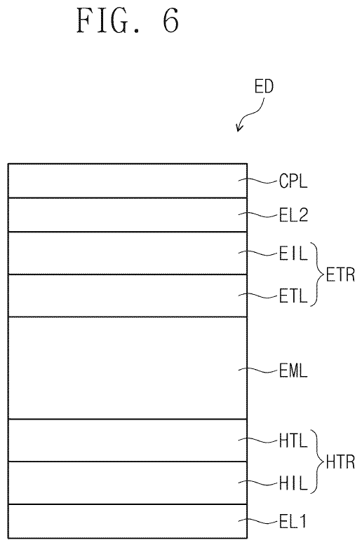

Compared with FIG. 3, FIG. 4 illustrates a cross-sectional view of a light emitting element ED of one or more embodiments, in which a hole transport region HTR includes a hole injection layer HIL and a hole transport layer HTL, and an electron transport region ETR includes an electron injection layer EIL and an electron transport layer ETL. In some embodiments, compared with FIG. 3, FIG. 5 illustrates a cross-sectional view of a light emitting element ED of one or more embodiments, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 4, FIG. 6 illustrates a cross-sectional view of a light emitting element ED of one or more embodiments including a capping layer CPL arranged on a second electrode EL2.

The light emitting element ED of one or more embodiments may include the amine compound of one or more embodiments, which will be explained later, in a hole transport region HTR. The light emitting element ED of one or more embodiments may include the amine compound of one or more embodiments in at least one of the hole injection layer HIL, the hole transport layer HTL and the electron blocking layer EBL of the hole transport region HTR. For example, in the light emitting element ED of one or more embodiments, the hole transport layer HTL may include the amine compound of one or more embodiments.

The first electrode EL1 has conductivity (e.g., is a conductor). The first electrode EL1 may be formed of a metal material, a metal alloy, or a conductive compound. The first electrode EL1 may be an anode or a cathode. However, one or more embodiments of the present disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn, and Zn, a compound of two or more selected from among these, a mixture of two or more selected from among these, or an oxide thereof.

If (e.g., when) the first electrode EL1 is the transmissive electrode, the first electrode EL1 may include a transparent metal oxide such as indium tin oxide (ITO), indium zinc oxide (IZO), zinc oxide (ZnO), or indium tin zinc oxide (ITZO). If (e.g., when) the first electrode EL1 is the transfiective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound or mixture thereof (e.g., a mixture of Ag and Mg). In one or more embodiments, the first electrode EL1 may have a multilayer structure including a reflective film or a transflective film formed of the herein-described materials, and a transparent conductive film formed of ITO, IZO, ZnO, ITZO, and/or the like. For example, the first electrode EL1 may have a three-layer structure of ITO/Ag/ITO, but one or more embodiments of the present disclosure is not limited thereto. In some embodiments, one or more embodiments of the disclosure is not limited thereto, and the first electrode EL1 may include the herein-described metal materials, combinations of at least two metal materials of the herein-described metal materials, oxides of the herein-described metal materials, and/or the like. The thickness of the first electrode EL1 may be from about 700 angstrom (A) to about 10,000 Å. For example, the thickness of the first electrode EL1 may be from about 1,000 Å to about 3,000 Å.

The hole transport region HTR is provided on the first electrode EL1. The hole transport region HTR may include at least one of a hole injection layer HIL, a hole transport layer HTL, and an electron blocking layer EBL. In some embodiments, though not shown, the hole transport region HTR may include multiple stacked hole transport layers.

The hole transport region HTR may have a single layer structure of the hole injection layer HIL or the hole transport layer HTL, or may have a single layer structure formed of a hole injection material and a hole transport material. In some embodiments, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron blocking layer EBL are stacked in order from the first electrode EL1, but one or more embodiments of the present disclosure is not limited thereto.

The thickness of the hole transport region HTR may be, for example, from about 50 Å to about 15,000 Å. The hole transport region HTR may be formed utilizing one or more suitable methods such as a vacuum deposition method, a spin coating method, a cast method, a Langmuir-Blodgett (LB) method, an inkjet printing method, a laser printing method, and a laser induced thermal imaging (LITI) method.

The light emitting element ED of one or more embodiments may include the amine compound of one or more embodiments in a hole transport region HTR. In the light emitting element ED of one or more embodiments, the hole transport region HTR includes at least one selected from among a hole injection layer HIL, a hole transport layer HTL and an electron blocking layer EBL, and may include the amine compound of one or more embodiments in at least one selected from among the hole injection layer HIL, the hole transport layer HTL and the electron blocking layer EBL. For example, the hole transport layer HTL may include the amine compound of one or more embodiments.

### Amine Compound

The amine compound of one or more embodiments includes an amine group and may include a first substituent, a second substituent and a third substituent, connected with (e.g., to) the amine group. For example, the amine compound of one or more embodiments may include a structure in which the first to third substituents are connected with (e.g., to) the nitrogen atom of the amine group.

The first substituent may include a carbazole moiety. The carbazole moiety may be connected with (e.g., to) the nitrogen atom of the amine group at position 1, position 2, position 3 or position 4 of the carbazole moiety. In one or more embodiments, the carbazole moiety may be connected with (e.g., to) the nitrogen atom of the amine group at position 8 corresponding to position 1, position 6 corresponding to position 2, position 6 corresponding to position 3 or position 5 corresponding to position 4. The first substituent may be directly bonded to the nitrogen atom of the amine group. The carbazole moiety in the first substituent may be connected with (e.g., to) a phenyl group substituted at position 9 of the carbazole moiety. For example, the carbazole moiety may be combined with a phenyl group substituted at the nitrogen atom of the carbazole moiety. A substituted phenyl group connected with (e.g., to) the nitrogen atom of the carbazole moiety, may refer to a phenylene linker substituted with a substituted or unsubstituted aryl group of 10 to 30 ring-forming carbon atoms in at least one position among the meta positions and para position with respect to the nitrogen atom of the carbazole moiety. In the present disclosure, the atomic (e.g., carbon) number (e.g., carbon atom position number) of the carbazole moiety is imparted as represented in Formula ST1.

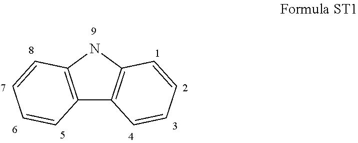 [SMILES: c1ccc2c(c1)[nH]c1ccccc12]

The second substituent may include a dibenzofuran moiety or a dibenzothiophene moiety. The second substituent may be directly bonded to the nitrogen atom of the amine group. In the present disclosure, the carbon number (e.g., carbon atom position number) of the second substituent may be imparted as represented in Formula ST2. In Formula ST2, X may be an oxygen atom (O) or a sulfur atom (S). In Formula ST2, if X is O, the second substituent may include a dibenzofuran moiety. In some embodiments, in Formula ST2, if X is S, the second substituent may include a dibenzothiophene moiety. For the convenience of explanation, substituents substituted at the dibenzofuran moiety or dibenzothiophene moiety are omitted in Formula ST2.

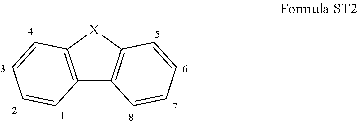

The third substituent may be an aryl group or a heteroaryl group. The third substituent may be directly bonded to the nitrogen atom of the amine group without a separate linker.

The amine compound of one or more embodiments may be a monoamine compound including a single amine group. The amine compound of one or more embodiments may be a monoamine compound in which only one amine group is present without forming a ring in a molecular structure.

In one or more embodiments, the amine compound may be represented by Formula 1. In Formula 1, a carbazole moiety in which Ar² and Ar³ are connected with the nitrogen atom of a carbazole moiety via a phenylene linker may correspond to the first substituent. In some embodiments, the dibenzofuran moiety or dibenzothiophene moiety including X which is an oxygen atom (O) or a sulfur atom (S) as a ring-forming atom, may correspond to the second substituent, and Ar¹ may correspond to the third substituent.

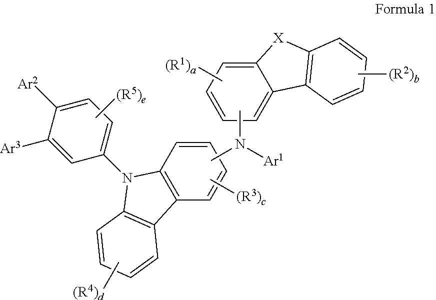

In Formula 1, X may be O or S. If X is O, the amine compound represented by Formula 1 may include a dibenzofuran moiety. If X is S, the amine compound represented by Formula 1 may include a dibenzothiophene moiety.

In Formula 1, Ar¹ may be a substituted or unsubstituted aryl group of 6 to 30 ring-forming carbon atoms, or a substituted or unsubstituted heteroaryl group of 2 to 30 ring-forming carbon atoms. For example, Ar¹ may be a substituted or unsubstituted phenyl group, a substituted or unsubstituted biphenyl group, a substituted or unsubstituted terphenyl group, a substituted or unsubstituted dibenzofuran group, or a substituted or unsubstituted dibenzothiophene group. However, in Formula 1, a case where Ar¹ comprises a moiety of

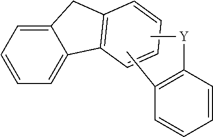

(Y is O or S), may be excluded. For example, in Formula 1, a case in which Ar¹ comprises a moiety of

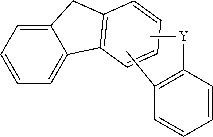

directly connected with the nitrogen atom of the amine compound and Y is O or S, is excluded. Because the amine compound of one or more embodiments, represented by Formula 1 does not include

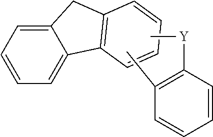

(Y is O or S) as Ar¹, hole transport properties may be improved, and improved emission efficiency and element lifetime may be shown.

In Formula 1, Ar² and Ar³ may each independently be a hydrogen atom, a deuterium atom, or a substituted or unsubstituted aryl group of 6 to 30 ring-forming carbon atoms. In this case, at least one selected from among Ar² and Ar³ may be a substituted or unsubstituted aryl group of 10 to 30 ring-forming carbon atoms. In Formula 1, the aryl group of 10 to 30 ring-forming carbon atoms may refer to an entire aryl group connected with the phenylene group in at least one selected from among the para position and meta positions with respect to the nitrogen atom of a carbazole moiety. For example, in Formula 1, the aryl group of 10 to 30 ring-forming carbon atoms, connected with the phenylene linker of the carbazole moiety may include

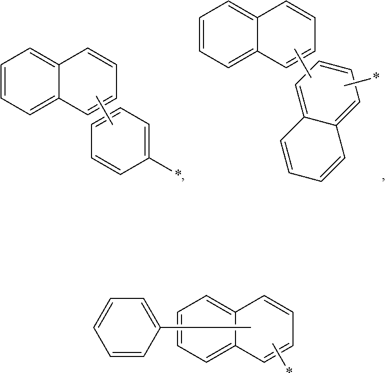

and/or the like, together with a naphthyl group, a phenanthrenyl group, a triphenylenyl group, a pyrenyl group and/or the like, and one or more embodiments of the present disclosure is not limited to any one or more embodiments.

In one or more embodiments, one selected from among Ar² and Ar³ may be a substituted or unsubstituted aryl group of 10 to 30 ring-forming carbon atoms, and the remainder may be a hydrogen atom, a deuterium atom, or a substituted or unsubstituted aryl group of 6 to 30 ring-forming carbon atoms. In some embodiments, both (e.g., simultaneously) Ar² and Ar³ may be substituted or unsubstituted aryl groups of 10 to 30 ring-forming carbon atoms.

For example, at least one selected from among Ar² and Ar³ may be a substituted or unsubstituted naphthyl group, a substituted or unsubstituted phenanthrenyl group, a substituted or unsubstituted triphenylenyl group, or a substituted or unsubstituted pyrenyl group. At least one selected from among Ar² and Ar³ may be represented by one selected from among the substituents in Substituent Group 1. However, one or more embodiments of the present disclosure is not limited thereto. In Substituent Group 1, “D” may be a deuterium atom, and * represents a site bonding to Formula 1.

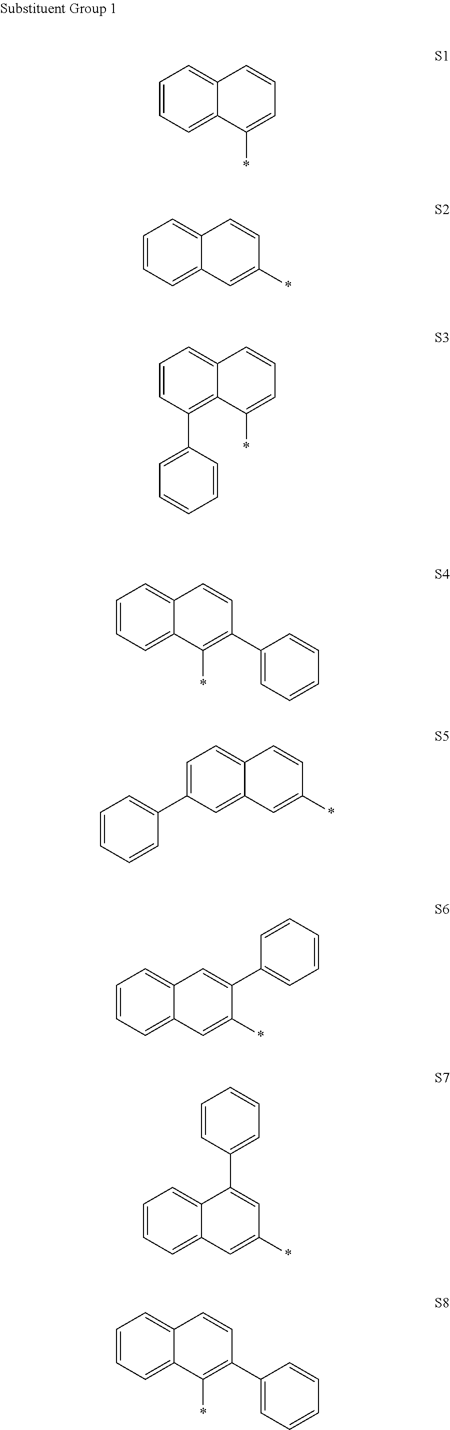

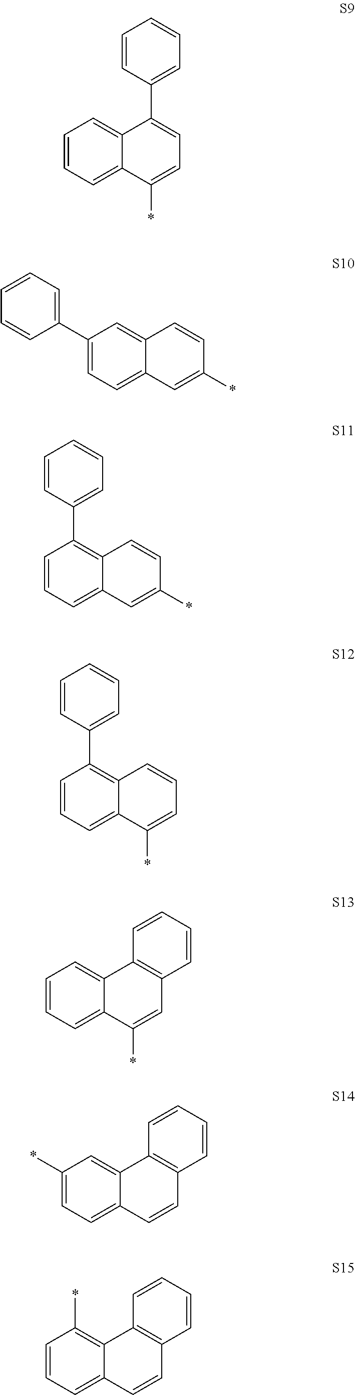

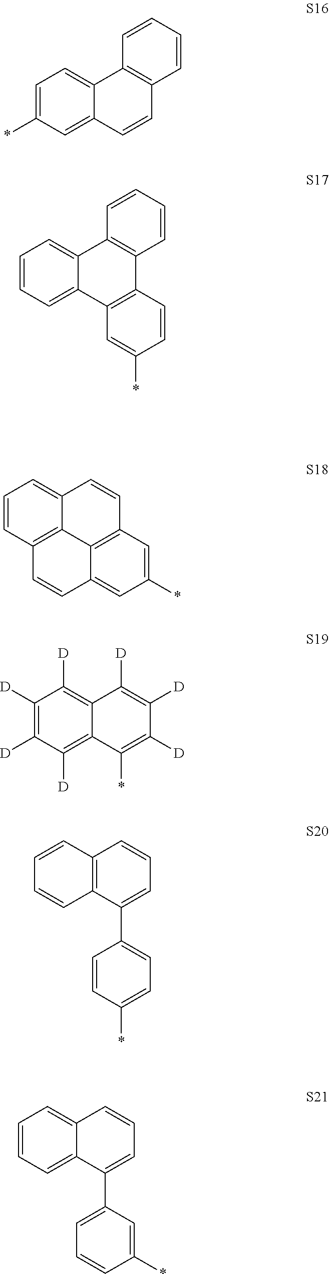 [SMILES: *c1ccc2c(ccc3ccccc32)c1 ; *c1ccc2c3ccccc3c3ccccc3c2c1 ; *c1cc2ccc3cccc4ccc(c1)c2c34 ; *c1c([2H])c([2H])c([2H])c2c([2H])c([2H])c([2H])c([2H])c12 ; *c1ccc(-c2cccc3ccccc23)cc1 ; *c1cccc(-c2cccc3ccccc23)c1]

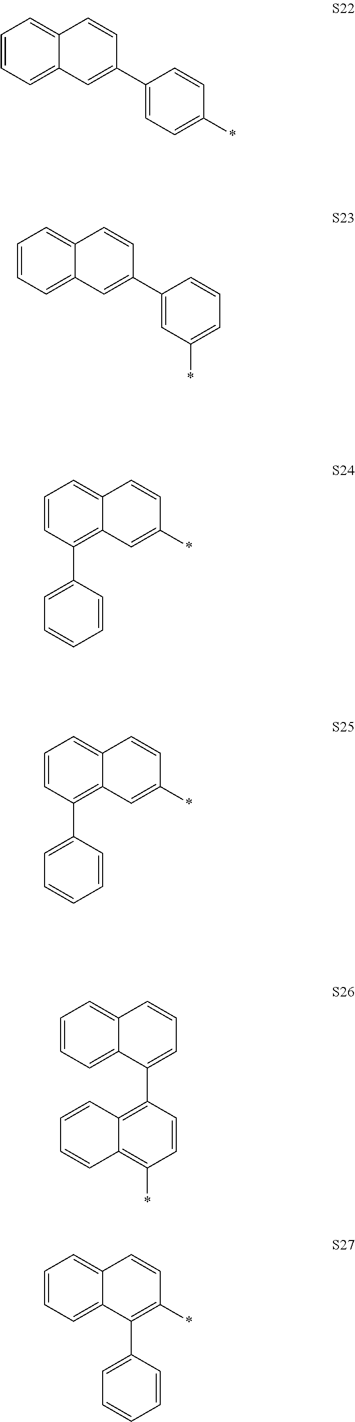 [SMILES: *c1ccc(-c2ccc3ccccc3c2)cc1 ; *c1cccc(-c2ccc3ccccc3c2)c1 ; *c1ccc2cccc(-c3ccccc3)c2c1 ; *c1ccc2cccc(-c3ccccc3)c2c1 ; *c1ccc(-c2cccc3ccccc23)c2ccccc12 ; *c1ccc2ccccc2c1-c1ccccc1]

In Formula 1, R¹ to R⁵ may each independently be a hydrogen atom, a deuterium atom, a halogen atom, a substituted or unsubstituted alkyl group of 1 to 15 carbon atoms, a substituted or unsubstituted aryl group of 6 to 30 ring-forming carbon atoms, or a substituted or unsubstituted heteroaryl group of 2 to 30 ring-forming carbon atoms. For example, R¹ to R⁵ may each independently be a hydrogen atom, a deuterium atom, a halogen atom, a substituted or unsubstituted methyl group or a substituted or unsubstituted phenyl group, but one or more embodiments of the present disclosure is not limited thereto.

In one or more embodiments, R¹, R², R³ and R⁵ may each independently be a hydrogen atom, a deuterium atom, or a substituted or unsubstituted aryl group of 6 to 30 ring-forming carbon atoms. R⁴ may be a hydrogen atom, a deuterium atom, a halogen atom, a substituted or unsubstituted alkyl group of 1 to 15 carbon atoms, or a substituted or unsubstituted aryl group of 6 to 30 ring-forming carbon atoms.

In Formula 1, “a”, “c” and “e” may each independently be an integer of 0 to 3, and “b” and “d” may each independently be an integer of 0 to 4. In Formula 1, if “a” is 0, the amine compound of one or more embodiments may be unsubstituted with R¹. In Formula 1, a case where “a” is 3, and three R¹ are each (e.g., any or all) hydrogen atoms, may be the same as a case where “a” is 0. If “a” is an integer of 2 or more, multiple R¹ may be all the same, or at least one selected from among multiple R¹ may be different.

In Formula 1, if “b” is 0, the amine compound of one or more embodiments may be unsubstituted with R². In Formula 1, a case where “b” is 4, and four R² are each (e.g., any or all) hydrogen atoms, may be the same as a case where “b” is 0. If “b” is an integer of 2 or more, multiple R² may be all the same, or at least one selected from among multiple R² may be different.

In Formula 1, if “c” is 0, the amine compound of one or more embodiments may be unsubstituted with R³. In Formula 1, a case where “c” is 3, and three R³ are each (e.g., any or all) hydrogen atoms, may be the same as a case where “c” is 0. If “c” is an integer of 2 or more, multiple R³ may be all the same, or at least one selected from among multiple R³ may be different.

In Formula 1, if “d” is 0, the amine compound of one or more embodiments may be unsubstituted with R⁴. In Formula 1, a case where “d” is 4, and four R⁴ are each (e.g., any or all) hydrogen atoms, may be the same as a case where “d” is 0. If “d” is an integer of 2 or more, multiple R⁴ may be all the same, or at least one selected from among multiple R⁴ may be different.

In Formula 1, if “e” is 0, the amine compound of one or more embodiments may be unsubstituted with R⁵. In Formula 1, a case where “e” is 3, and three R⁵ are each (e.g., any or all) hydrogen atoms, may be the same as a case where “e” is 0. If “e” is an integer of 2 or more, multiple R⁵ may be all the same, or at least one selected from among multiple R⁵ may be different.

In the amine compound of one or more embodiments, represented by Formula 1, for a case where the nitrogen atom of the amine compound is connected with position 2 or position 6 of a carbazole moiety, while being connected with position 3 or position 6 of a dibenzothiophene moiety or a dibenzofuran moiety, and/or for a case where the nitrogen atom of the amine compound is connected with position 4 or position 5 of a carbazole moiety, while being connected with position 3 or position 6 of a dibenzothiophene moiety or a dibenzofuran moiety, a+b≥1 may be satisfied, and at least one selected from among R¹ and R² may not a hydrogen atom.

For example, the case where the nitrogen atom of the amine compound is connected with position 2 or position 6 of a carbazole moiety, while being connected with position 3 or position 6 of a dibenzothiophene moiety or a dibenzofuran moiety, may be represented by Formula 1-A.

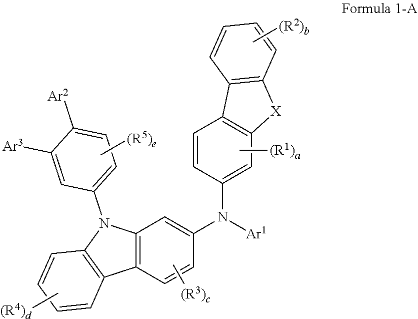

For example, the case where the nitrogen atom of the amine compound is connected with position 4 or position 5 of a carbazole moiety, while being connected with position 3 or position 6 of a dibenzothiophene moiety or a dibenzofuran moiety, may be represented by Formula 1-B.

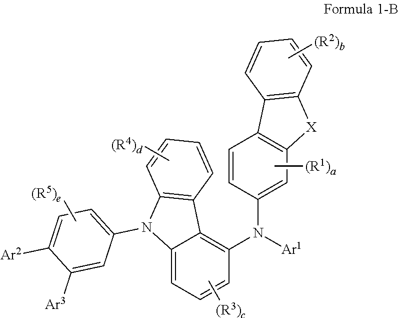

In Formula 1-A and Formula 1-B, the same definitions (e.g., contents) explained in Formula 1 may be applied for X, Ar¹ to Ar³, R¹ to R⁵, and “a” to “e”. However, if the amine compound of one or more embodiments is represented by Formula 1-A or Formula 1-B, a+b≥1 may be satisfied, and at least one selected from among R¹ and R² may not a hydrogen atom in Formula 1-A and Formula 1-B. For example, in Formula 1-A and Formula 1-B, at least one selected from among R¹ and R² may be a substituted or unsubstituted aryl group of 6 to 30 ring-forming carbon atoms, but one or more embodiments of the present disclosure is not limited thereto.

In the amine compound of one or more embodiments, represented by Formula 1, a case where the nitrogen atom of the amine compound is connected with position 3 of the carbazole moiety, R³ may be a hydrogen atom or a deuterium atom. For example, a case of Formula 1 where the nitrogen atom of the amine compound is connected at position 3 of the carbazole moiety may be represented by Formula 1-C.

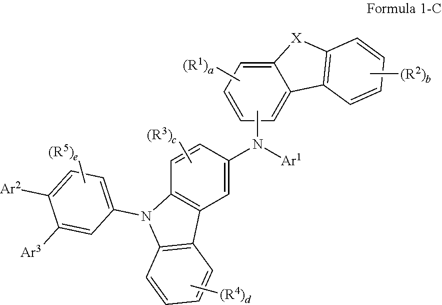

In Formula 1-C, the same definitions (e.g., contents) explained in Formula 1 may be applied for X, Ar¹ to Ar³, R¹, R², R⁴, R⁵, and “a” to “e”. However, if the amine compound of one or more embodiments is represented by Formula 1-C, R³ may be a hydrogen atom or a deuterium atom. For example, in Formula 1-C, R³ may be a hydrogen atom.

The amine compound of one or more embodiments may include a deuterium atom as a substituent. For example, in the amine compound represented by Formula 1, at least one selected from among Ar¹ to Ar³, and R¹ to R⁵ may include a deuterium atom or a substituent including a deuterium atom. However, this is merely an illustrative example, and one or more embodiments of the present disclosure is not limited thereto.

In one or more embodiments, the amine compound represented by Formula may be represented by Formula 2-1 or Formula 2-2. Formula 2-1 and Formula 2-2 correspond to Formula 1 where Ar¹ is embodied. In Formula 2-1 and Formula 2-2, the same definitions (e.g., contents) explained in Formula 1 may be applied for X, Ar², Ar³, R¹ to R⁵, and “a” to “e”.

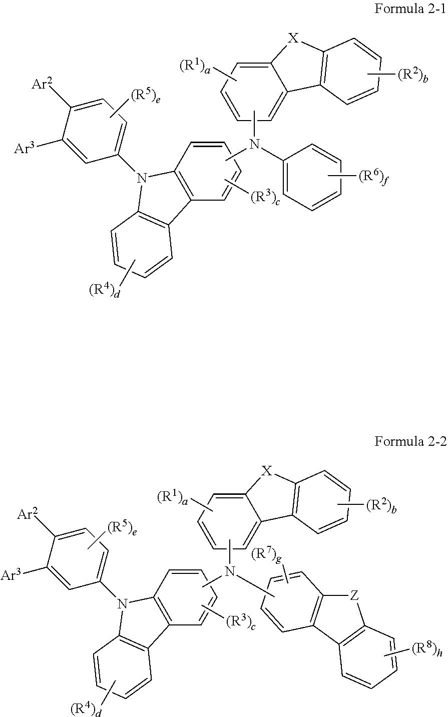

In Formula 2-1, R⁶ may be a hydrogen atom, a deuterium atom, a substituted or unsubstituted aryl group of 6 to 30 ring-forming carbon atoms, or a substituted or unsubstituted heteroaryl group of 2 to 30 ring-forming carbon atoms. For example, R⁶ may be a hydrogen atom, a deuterium atom, a substituted or unsubstituted phenyl group, a substituted or unsubstituted biphenyl group, a substituted or unsubstituted dibenzofuran group, or a substituted or unsubstituted dibenzothiophene group.

In Formula 2-1, “f” may be an integer of 0 to 5. In Formula 2-1, if “f” is 0, the amine compound of one or more embodiments may be unsubstituted with R⁶. In Formula 2-1, a case where “f” is 5, and five R⁶ are each (e.g., all) hydrogen atoms, may be the same as a case where “f” is 0. If “f” is an integer of 2 or more, multiple R⁶ may be all the same, or at least one selected from among multiple R⁶ may be different.

In Formula 2-2, Z may be O or S. If Z is 0, the amine compound represented by Formula 2-2 may include a dibenzofuran moiety as a third substituent. If Z is S, the amine compound represented by Formula 2-2 may include a dibenzothiophene moiety as a third substituent.

In Formula 2-2, R⁷ and R⁸ may each independently be a hydrogen atom or a deuterium atom. “g” may be an integer of 0 to 3, and “h” may be an integer of 0 to 4. In Formula 2-2, if “g” is 0, the amine compound of one or more embodiments may include a dibenzofuran moiety or a dibenzothiophene moiety which is unsubstituted with R⁷ as the third substituent. In Formula 2-2, a case where “g” is 3, and three R⁷ are each (e.g., all) hydrogen atoms, may be the same as a case where “g” is 0. If “g” is an integer of 2 or more, multiple R⁷ may be each (e.g., all) the same, or at least one selected from among multiple R⁷ may be different.

In Formula 2-2, if “h” is 0, the amine compound of one or more embodiments may include a dibenzofuran moiety or a dibenzothiophene moiety which is unsubstituted with R⁸ as a third substituent. In Formula 2-2, a case where “h” is 4, and four R⁸ are each (e.g., all) hydrogen atoms, may be the same as a case where “h” is 0. If “h” is an integer of 2 or more, multiple R⁸ may be each (e.g., all) the same, or at least one selected from among multiple R⁸ may be different.

In one or more embodiments, the amine compound represented by Formula may be represented by Formula 3-1 or Formula 3-2. Formula 3-1 and Formula 3-2 represent Formula 1 where one selected from among Ar² and Ar³ is a substituted or unsubstituted naphthyl group. Formula 3-1 may correspond to Formula 1 where Ar² is a substituted or unsubstituted naphthyl group, and Formula 3-2 may correspond to Formula 1 where Ar³ is a substituted or unsubstituted naphthyl group.

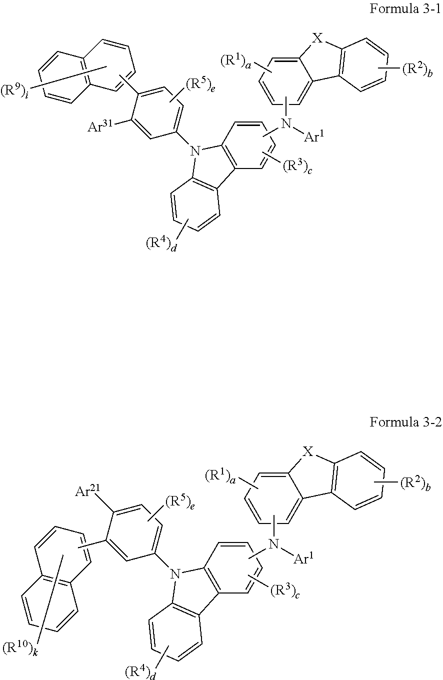

In Formula 3-1 and Formula 3-2, the same definitions (e.g., contents) explained in Formula 1 may be applied for X, Ar¹, R¹ to R⁵, and “a” to “e”.

In Formula 3-1 and Formula 3-2, R⁹, R¹⁰, Ar²¹ and Ar³¹ may each independently be a hydrogen atom, a deuterium atom, or a substituted or unsubstituted aryl group of 6 to 30 ring-forming carbon atoms. For example, R⁹, R¹⁰, Ar²¹ and Ar³¹ may each independently be a hydrogen atom, a deuterium atom, or a substituted or unsubstituted phenyl group.

In Formula 3-1 and Formula 3-2, “i” and “k” may each independently be an integer of 0 to 7. If “i” is 0, in the amine compound of one or more embodiments, a naphthyl group which is unsubstituted with R⁹ may be connected with a linker at the para relation with respect to the nitrogen atom of a carbazole moiety. In Formula 3-1, a case where “i” is 7, and seven R⁹ are each (e.g., all) hydrogen atoms, may be the same as a case where “i” is 0. If “i” is an integer of 2 or more, multiple R⁹ may be each (e.g., all) the same, or at least one selected from among multiple R⁹ may be different. For example, “i” may be 7, one selected from among seven R⁹ may be an unsubstituted phenyl group, and the remaining six R⁹ may be hydrogen atoms. If “k” is 0, in the amine compound of one or more embodiments, a naphthyl group which is unsubstituted with R¹⁰ may be connected with (e.g., to) a phenylene linker at the para relation with respect to the nitrogen atom of a carbazole moiety. In Formula 3-2, a case where “k” is 7, and seven R¹⁰ are each (e.g., all) hydrogen atoms, may be the same as a case where “k” is 0. If “k” is an integer of 2 or more, multiple R¹⁰ may be each (e.g., all) the same, or at least one selected from among multiple R¹⁰ may be different. For example, “k” may be 7, one selected from among seven R¹⁰ may be an unsubstituted phenyl group, and the remaining six R¹⁰ may be hydrogen atoms.

In one or more embodiments, the amine compound represented by Formula may be represented by one selected from among Formula 4-1 to Formula 4-4. In Formula 4-1 to Formula 4-4, the same definitions (e.g., contents) explained in Formula may be applied for X, and Ar¹ to Ar³.

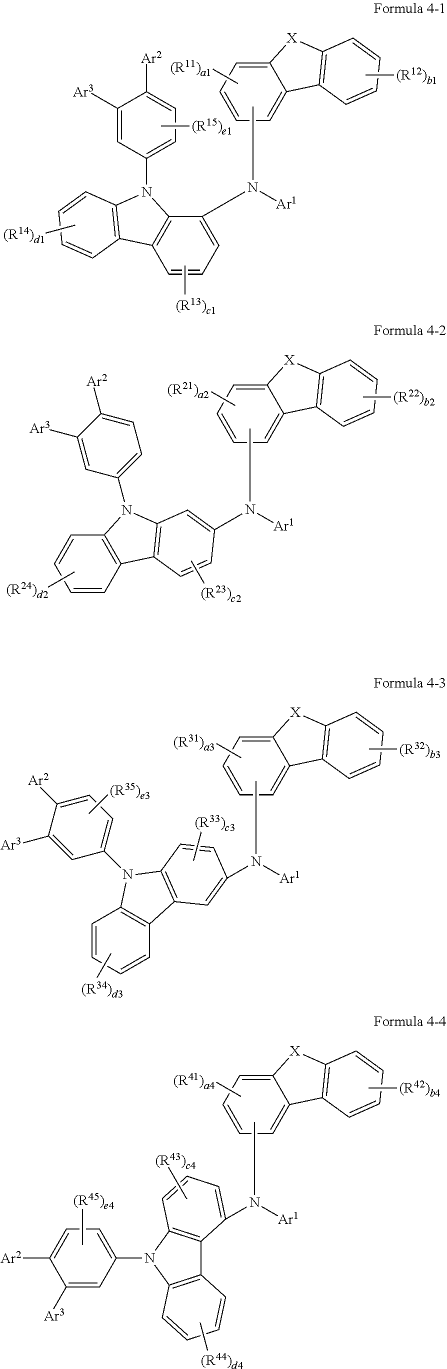

In Formula 4-1 to Formula 4-4, R¹¹ to R¹³, R²¹ to R²³, R³¹ to R³³, R⁴¹, and R⁴² may each independently be a hydrogen atom, a deuterium atom, or a substituted or unsubstituted aryl group of 6 to 30 ring-forming carbon atoms. For example, R¹¹ to R¹³, R²¹ to R²³, R³¹ to R³³, R⁴¹, and R⁴² may each independently be a hydrogen atom, a deuterium atom, or a substituted or unsubstituted phenyl group.

In Formula 4-1 to Formula 4-4, R¹⁴, R²⁴, R³⁴, and R⁴⁴ may each independently be a hydrogen atom, a deuterium atom, a halogen atom, a substituted or unsubstituted alkyl group of 1 to 15 carbon atoms, or a substituted or unsubstituted aryl group of 6 to 30 ring-forming carbon atoms. For example, R¹⁴, R²⁴, R³⁴, and R⁴⁴ may each independently be a hydrogen atom, a deuterium atom, a halogen atom, a substituted or unsubstituted methyl group or a substituted or unsubstituted phenyl group.

In Formula 4-1 to Formula 4-4, R¹⁵, R²⁵, R³⁵, R⁴³ and R⁴⁵ may each independently be a hydrogen atom or a deuterium atom, but one or more embodiments of the present disclosure is not limited thereto.

In Formula 4-1 to Formula 4-4, a1 to a4, c1 to c4, and e1 to e4 may each independently be an integer of 0 to 3. b1 to b4, and d1 to d4 may each independently be an integer of 0 to 4.

In Formula 4-1 to Formula 4-4, if a1, a2, a3 and a4 are 0, the amine compound of one or more embodiments may be unsubstituted with R¹¹, R²¹, R³¹ and R⁴¹, respectively. In Formula 4-1, a case where a1 is 3, and three R¹¹ are all hydrogen atoms, may be the same as a case where a1 is 0. If a1 is an integer of 2 or more, multiple R¹¹ may be each (e.g., all) the same, or at least one selected from among multiple R¹¹ may be different. The definition of (e.g., explanation on) a1 may be applied for a2, a3 and a4 in substantially the same way.

In Formula 4-1 to Formula 4-4, if b1, b2, b3 and b4 are 0, the amine compound of one or more embodiments may be unsubstituted with R¹², R²², R³² and R⁴², respectively. In Formula 4-1, a case where b1 is 4, and four R¹² are each (e.g., all) hydrogen atoms, may be the same as a case where b1 is 0. If b1 is an integer of 2 or more, multiple R¹² may be each (e.g., all) the same, or at least one selected from among multiple R¹² may be different. The definition of (e.g., explanation on) b1 may be applied for b2, b3 and b4 in substantially the same way.

In Formula 4-1 to Formula 4-4, if c1, c2, c3 and c4 are 0, the amine compound of one or more embodiments may be unsubstituted with R¹³, R²³, R³³ and R⁴³, respectively. In Formula 4-1, a case where c1 is 3, and three R¹³ are all hydrogen atoms, may be the same as a case where c1 is 0. If c1 is an integer of 2 or more, multiple R¹³ may be each (e.g., all) the same, or at least one selected from among multiple R¹³ may be different. The definition of (e.g., explanation on) c1 may be applied for c2, c3 and c4 in substantially the same way.

In Formula 4-1 to Formula 4-4, if d1, d2, d3 and d4 are 0, the amine compound of one or more embodiments may be unsubstituted with R¹⁴, R²⁴, R³⁴ and R⁴⁴, respectively. In Formula 4-1, a case where d1 is 4, and four R¹⁴ are each (e.g., all) hydrogen atoms, may be the same as a case where d1 is 0. If d1 is an integer of 2 or more, multiple R¹⁴ may be each (e.g., all) the same, or at least one selected from among multiple R¹⁴ may be different. The definition of (e.g., explanation on) d1 may be applied for d2, d3 and d4 in substantially the same way.

In Formula 4-1 to Formula 4-4, if e1, e2, e3 and e4 are 0, the amine compound of one or more embodiments may be unsubstituted with R¹⁵, R²⁵, R³⁵ and R⁴⁵, respectively. In Formula 4-1, a case where e1 is 3, and three R¹⁵ are all hydrogen atoms, may be the same as a case where e1 is 0. If e1 is an integer of 2 or more, multiple R¹⁵ may be each (e.g., all) the same, or at least one selected from among multiple R¹⁵ may be different. The definition of (e.g., explanation on) e1 may be applied for e2, e3 and e4 in substantially the same way.

In one or more embodiments, in Formula 4-2, if the nitrogen atom of the amine compound is connected with position 3 or position 6 of a dibenzothiophene moiety or a dibenzofuran moiety, a2+b2≥1 may be satisfied, and at least one selected from among R²¹ and R²² may be a substituted or unsubstituted aryl group of 6 to 30 ring-forming carbon atoms. For example, in Formula 4-2, if the nitrogen atom of the amine compound is connected with position 3 or position 6 of a dibenzothiophene moiety or a dibenzofuran moiety, a2+b2≥1 may be satisfied, and at least one selected from among R²¹ and R²² may be a substituted or unsubstituted phenyl group.

In Formula 4-4, if the nitrogen atom of the amine compound is connected with position 3 or position 6 of a dibenzothiophene moiety or a dibenzofuran moiety, a4+b4≥1 may be satisfied, and at least one selected from among R⁴¹ and R⁴² may be a substituted or unsubstituted aryl group of 6 to 30 ring-forming carbon atoms. For example, in Formula 4-4, if the nitrogen atom of the amine compound is connected with position 3 or position 6 of a dibenzothiophene moiety or a dibenzofuran moiety, a4+b4≥1 may be satisfied, and at least one selected from among R⁴¹ and R⁴² may be a substituted or unsubstituted phenyl group.

The amine compound of one or more embodiments, represented by Formula may be represented by one (e.g., any one) selected from among the compounds in Compound Group 1. The hole transport region HTR of the light emitting element ED of one or more embodiments may include at least one selected from among the amine compounds shown in Compound Group 1. For example, at least one selected from among the amine compounds shown Compound Group 1 may be included in the electron blocking layer EBL of the light emitting element ED. In Compound Group 1, “D” is a deuterium atom.

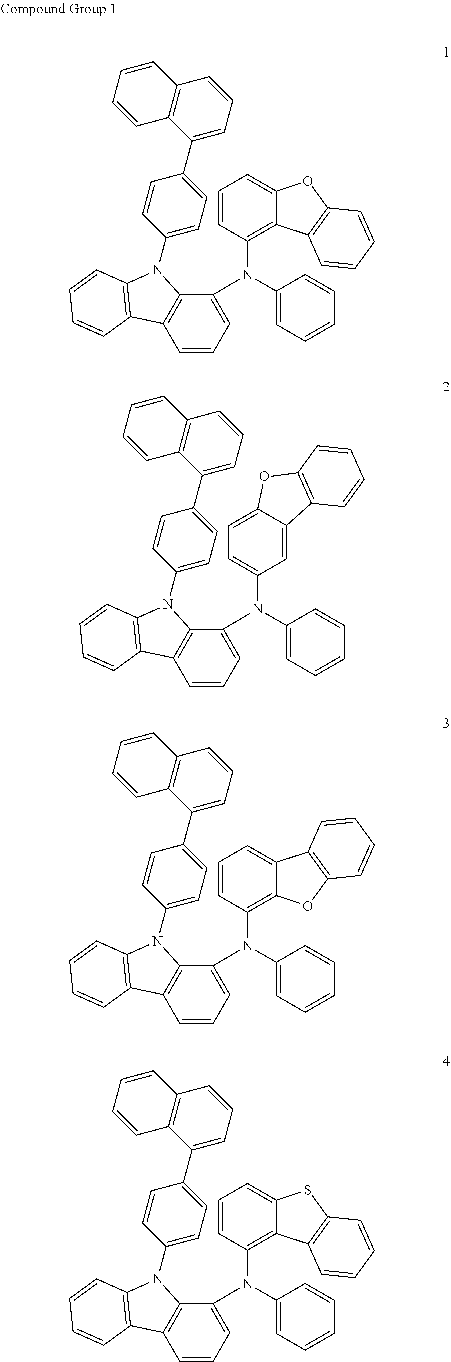

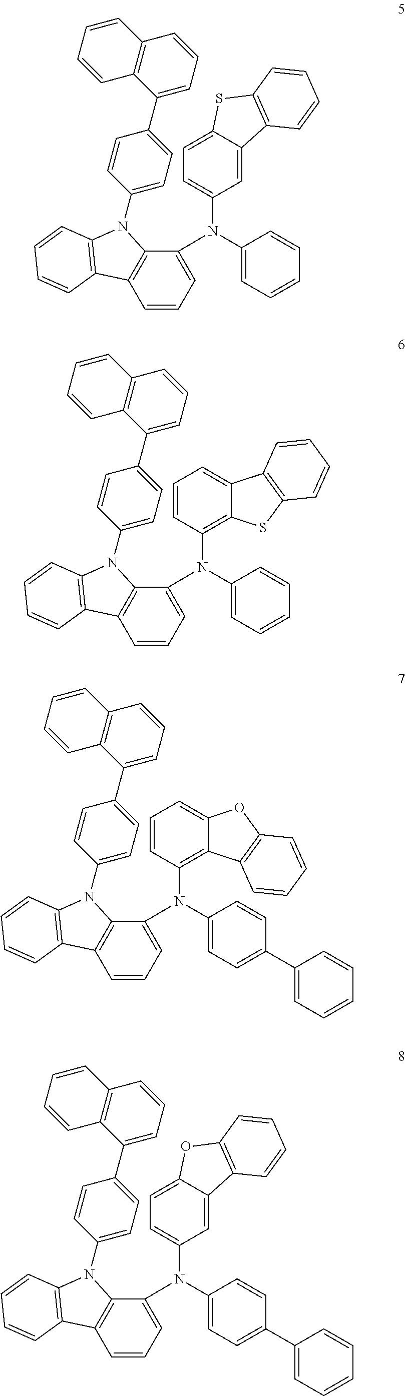 [SMILES: c1ccc(N(c2ccc3sc4ccccc4c3c2)c2cccc3c4ccccc4n(-c4ccc(-c5cccc6ccccc56)cc4)c23)cc1 ; c1ccc(N(c2cccc3c2sc2ccccc23)c2cccc3c4ccccc4n(-c4ccc(-c5cccc6ccccc56)cc4)c23)cc1 ; c1ccc(-c2ccc(N(c3cccc4oc5ccccc5c34)c3cccc4c5ccccc5n(-c5ccc(-c6cccc7ccccc67)cc5)c34)cc2)cc1 ; c1ccc(-c2ccc(N(c3ccc4oc5ccccc5c4c3)c3cccc4c5ccccc5n(-c5ccc(-c6cccc7ccccc67)cc5)c34)cc2)cc1]

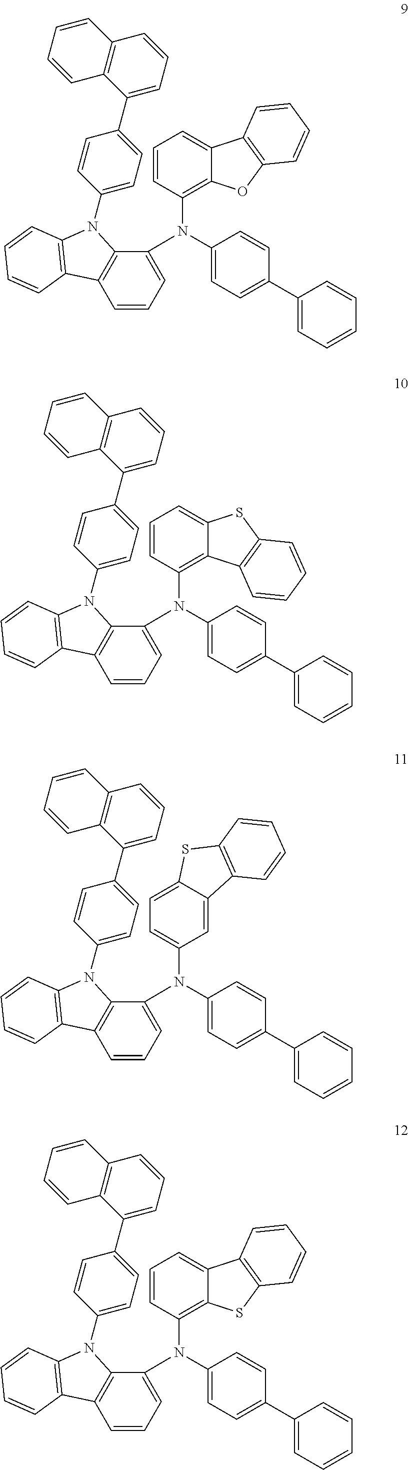 [SMILES: c1ccc(-c2ccc(N(c3cccc4c3oc3ccccc34)c3cccc4c5ccccc5n(-c5ccc(-c6cccc7ccccc67)cc5)c34)cc2)cc1 ; c1ccc(-c2ccc(N(c3cccc4sc5ccccc5c34)c3cccc4c5ccccc5n(-c5ccc(-c6cccc7ccccc67)cc5)c34)cc2)cc1 ; c1ccc(-c2ccc(N(c3ccc4sc5ccccc5c4c3)c3cccc4c5ccccc5n(-c5ccc(-c6cccc7ccccc67)cc5)c34)cc2)cc1 ; c1ccc(-c2ccc(N(c3cccc4c3sc3ccccc34)c3cccc4c5ccccc5n(-c5ccc(-c6cccc7ccccc67)cc5)c34)cc2)cc1]

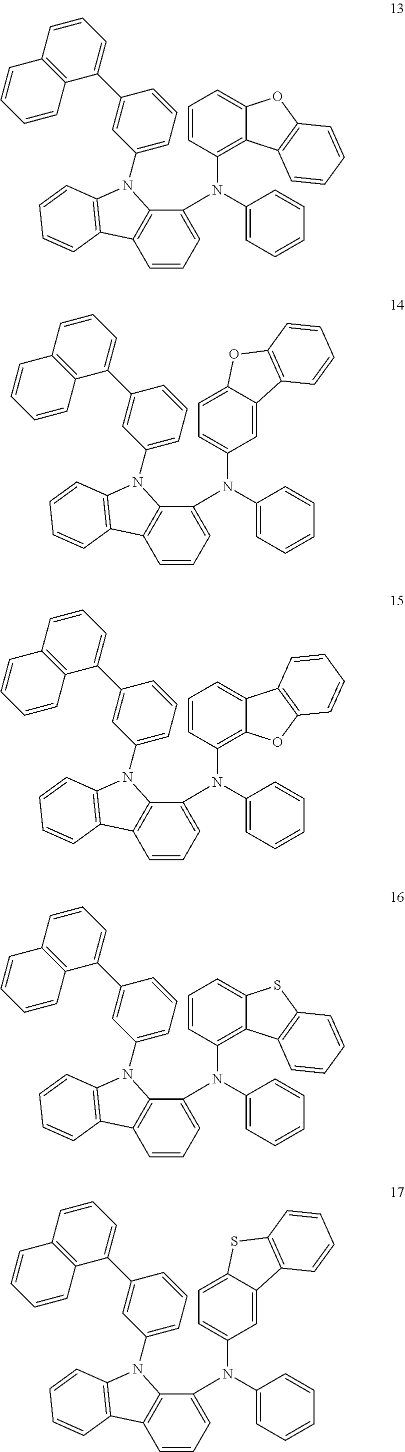 [SMILES: c1ccc(N(c2cccc3oc4ccccc4c23)c2cccc3c4ccccc4n(-c4cccc(-c5cccc6ccccc56)c4)c23)cc1 ; c1ccc(N(c2ccc3oc4ccccc4c3c2)c2cccc3c4ccccc4n(-c4cccc(-c5cccc6ccccc56)c4)c23)cc1 ; c1ccc(N(c2cccc3c2oc2ccccc23)c2cccc3c4ccccc4n(-c4cccc(-c5cccc6ccccc56)c4)c23)cc1 ; c1ccc(N(c2cccc3sc4ccccc4c23)c2cccc3c4ccccc4n(-c4cccc(-c5cccc6ccccc56)c4)c23)cc1 ; c1ccc(N(c2ccc3sc4ccccc4c3c2)c2cccc3c4ccccc4n(-c4cccc(-c5cccc6ccccc56)c4)c23)cc1]

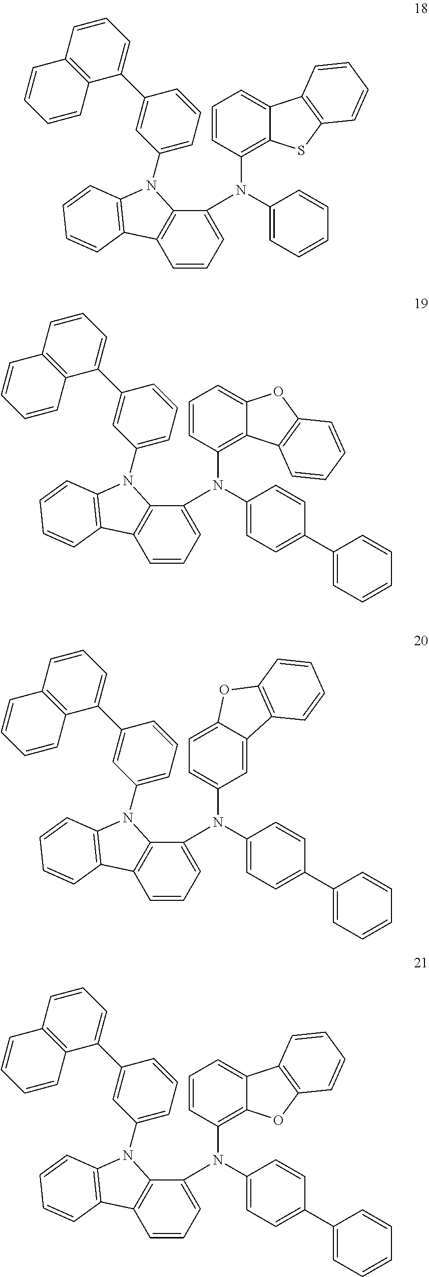 [SMILES: c1ccc(N(c2cccc3c2sc2ccccc23)c2cccc3c4ccccc4n(-c4cccc(-c5cccc6ccccc56)c4)c23)cc1 ; c1ccc(-c2ccc(N(c3cccc4oc5ccccc5c34)c3cccc4c5ccccc5n(-c5cccc(-c6cccc7ccccc67)c5)c34)cc2)cc1 ; c1ccc(-c2ccc(N(c3ccc4oc5ccccc5c4c3)c3cccc4c5ccccc5n(-c5cccc(-c6cccc7ccccc67)c5)c34)cc2)cc1 ; c1ccc(-c2ccc(N(c3cccc4c3oc3ccccc34)c3cccc4c5ccccc5n(-c5cccc(-c6cccc7ccccc67)c5)c34)cc2)cc1]

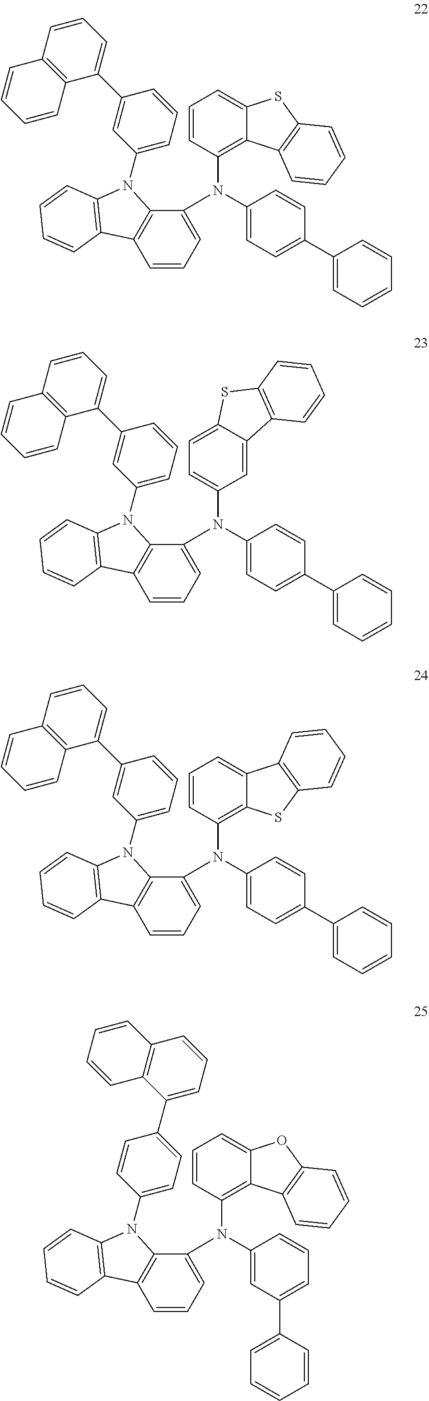 [SMILES: c1ccc(-c2ccc(N(c3cccc4sc5ccccc5c34)c3cccc4c5ccccc5n(-c5cccc(-c6cccc7ccccc67)c5)c34)cc2)cc1 ; c1ccc(-c2ccc(N(c3ccc4sc5ccccc5c4c3)c3cccc4c5ccccc5n(-c5cccc(-c6cccc7ccccc67)c5)c34)cc2)cc1 ; c1ccc(-c2ccc(N(c3cccc4c3sc3ccccc34)c3cccc4c5ccccc5n(-c5cccc(-c6cccc7ccccc67)c5)c34)cc2)cc1 ; c1ccc(-c2cccc(N(c3cccc4oc5ccccc5c34)c3cccc4c5ccccc5n(-c5ccc(-c6cccc7ccccc67)cc5)c34)c2)cc1]

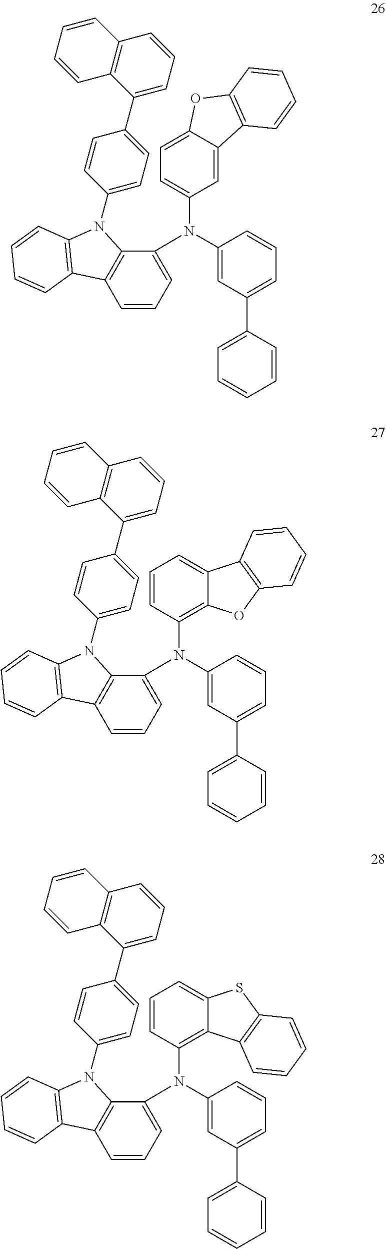 [SMILES: c1ccc(-c2cccc(N(c3ccc4oc5ccccc5c4c3)c3cccc4c5ccccc5n(-c5ccc(-c6cccc7ccccc67)cc5)c34)c2)cc1 ; c1ccc(-c2cccc(N(c3cccc4c3oc3ccccc34)c3cccc4c5ccccc5n(-c5ccc(-c6cccc7ccccc67)cc5)c34)c2)cc1 ; c1ccc(-c2cccc(N(c3cccc4sc5ccccc5c34)c3cccc4c5ccccc5n(-c5ccc(-c6cccc7ccccc67)cc5)c34)c2)cc1]

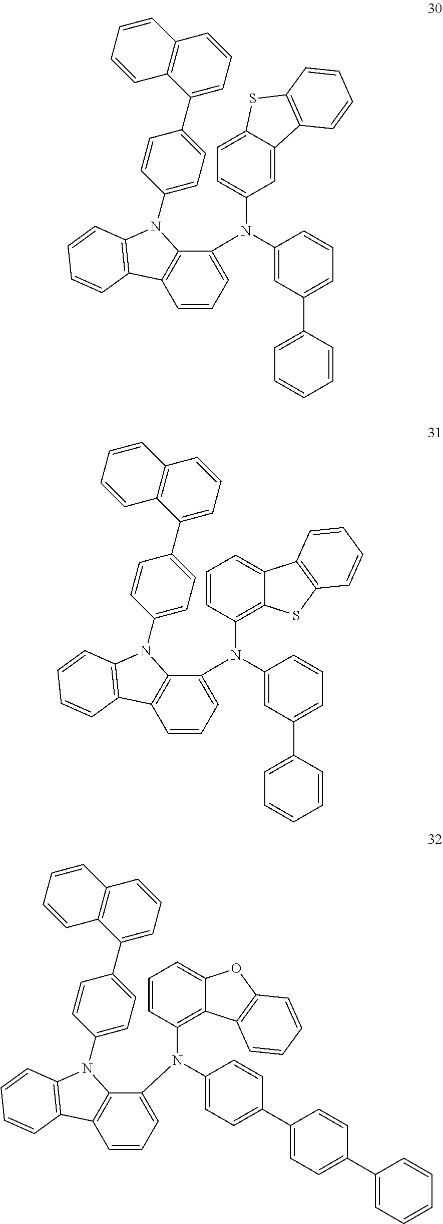 [SMILES: c1ccc(-c2cccc(N(c3ccc4sc5ccccc5c4c3)c3cccc4c5ccccc5n(-c5ccc(-c6cccc7ccccc67)cc5)c34)c2)cc1 ; c1ccc(-c2cccc(N(c3cccc4c3sc3ccccc34)c3cccc4c5ccccc5n(-c5ccc(-c6cccc7ccccc67)cc5)c34)c2)cc1 ; c1ccc(-c2ccc(-c3ccc(N(c4cccc5oc6ccccc6c45)c4cccc5c6ccccc6n(-c6ccc(-c7cccc8ccccc78)cc6)c45)cc3)cc2)cc1]

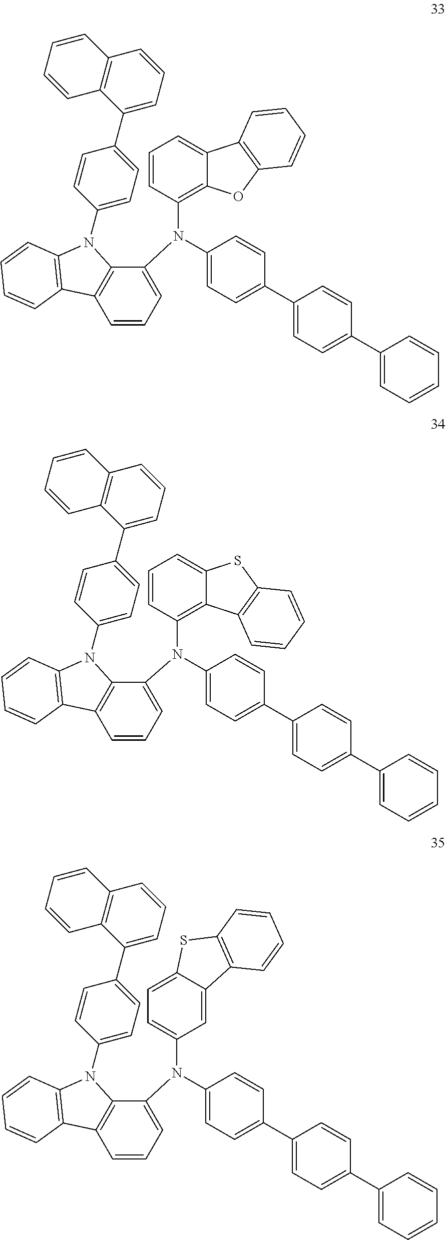 [SMILES: c1ccc(-c2ccc(-c3ccc(N(c4cccc5c4oc4ccccc45)c4cccc5c6ccccc6n(-c6ccc(-c7cccc8ccccc78)cc6)c45)cc3)cc2)cc1 ; c1ccc(-c2ccc(-c3ccc(N(c4cccc5sc6ccccc6c45)c4cccc5c6ccccc6n(-c6ccc(-c7cccc8ccccc78)cc6)c45)cc3)cc2)cc1 ; c1ccc(-c2ccc(-c3ccc(N(c4ccc5sc6ccccc6c5c4)c4cccc5c6ccccc6n(-c6ccc(-c7cccc8ccccc78)cc6)c45)cc3)cc2)cc1]

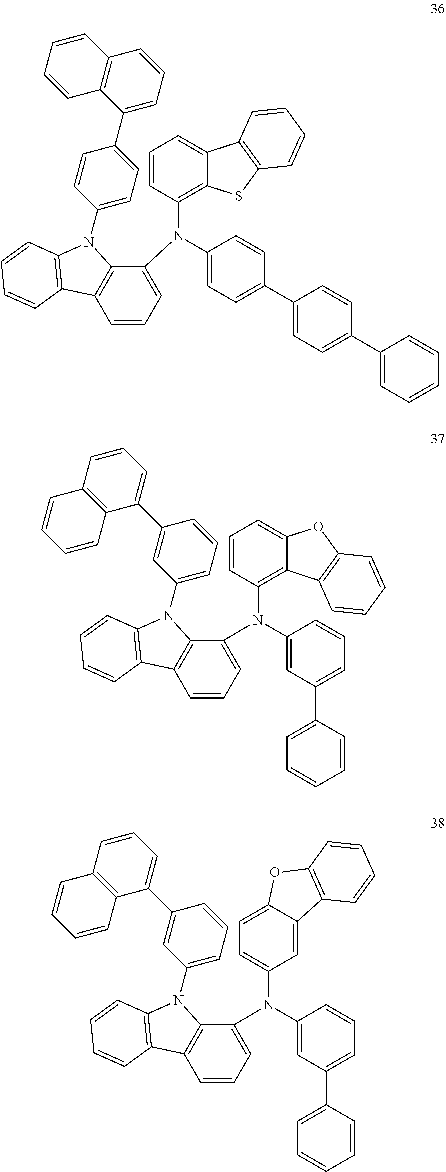 [SMILES: c1ccc(-c2ccc(-c3ccc(N(c4cccc5c4sc4ccccc45)c4cccc5c6ccccc6n(-c6ccc(-c7cccc8ccccc78)cc6)c45)cc3)cc2)cc1 ; c1ccc(-c2cccc(N(c3cccc4oc5ccccc5c34)c3cccc4c5ccccc5n(-c5cccc(-c6cccc7ccccc67)c5)c34)c2)cc1 ; c1ccc(-c2cccc(N(c3ccc4oc5ccccc5c4c3)c3cccc4c5ccccc5n(-c5cccc(-c6cccc7ccccc67)c5)c34)c2)cc1]

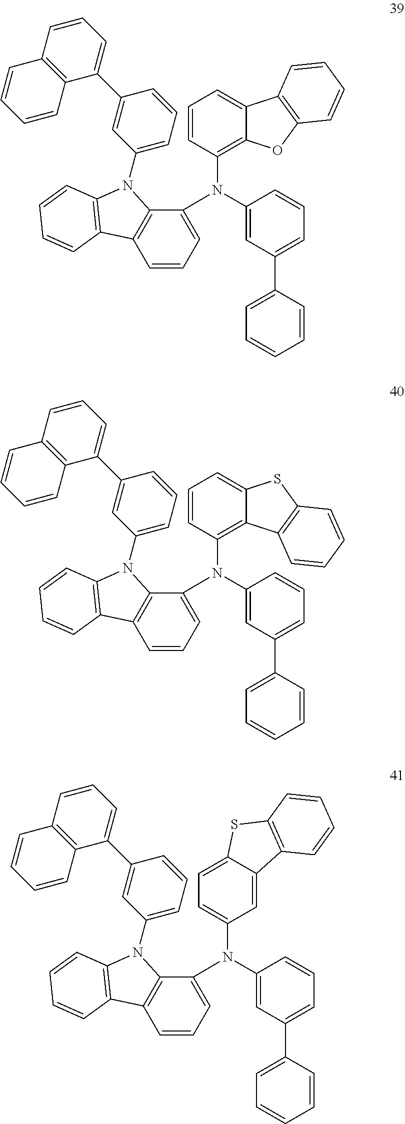 [SMILES: c1ccc(-c2cccc(N(c3cccc4c3oc3ccccc34)c3cccc4c5ccccc5n(-c5cccc(-c6cccc7ccccc67)c5)c34)c2)cc1 ; c1ccc(-c2cccc(N(c3cccc4sc5ccccc5c34)c3cccc4c5ccccc5n(-c5cccc(-c6cccc7ccccc67)c5)c34)c2)cc1 ; c1ccc(-c2cccc(N(c3ccc4sc5ccccc5c4c3)c3cccc4c5ccccc5n(-c5cccc(-c6cccc7ccccc67)c5)c34)c2)cc1]

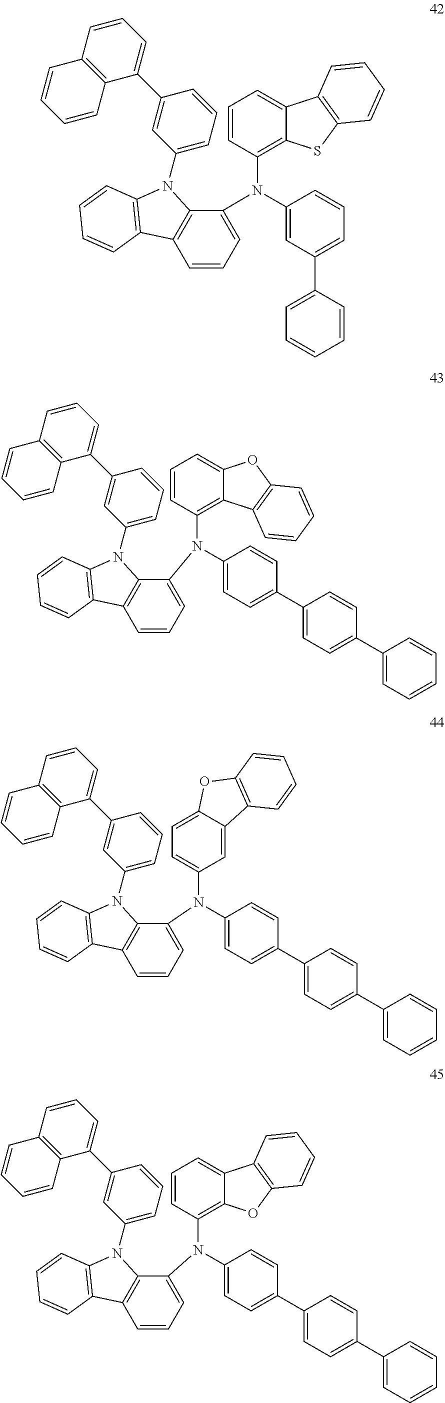 [SMILES: c1ccc(-c2cccc(N(c3cccc4c3sc3ccccc34)c3cccc4c5ccccc5n(-c5cccc(-c6cccc7ccccc67)c5)c34)c2)cc1 ; c1ccc(-c2ccc(-c3ccc(N(c4cccc5oc6ccccc6c45)c4cccc5c6ccccc6n(-c6cccc(-c7cccc8ccccc78)c6)c45)cc3)cc2)cc1 ; c1ccc(-c2ccc(-c3ccc(N(c4ccc5oc6ccccc6c5c4)c4cccc5c6ccccc6n(-c6cccc(-c7cccc8ccccc78)c6)c45)cc3)cc2)cc1 ; c1ccc(-c2ccc(-c3ccc(N(c4cccc5c4oc4ccccc45)c4cccc5c6ccccc6n(-c6cccc(-c7cccc8ccccc78)c6)c45)cc3)cc2)cc1]

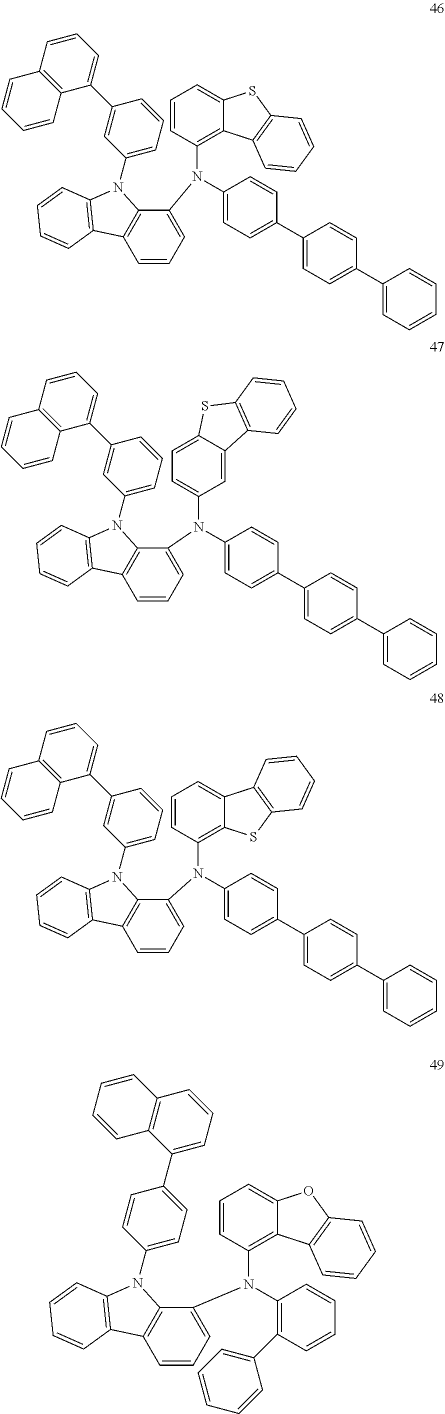 [SMILES: c1ccc(-c2ccc(-c3ccc(N(c4cccc5sc6ccccc6c45)c4cccc5c6ccccc6n(-c6cccc(-c7cccc8ccccc78)c6)c45)cc3)cc2)cc1 ; c1ccc(-c2ccc(-c3ccc(N(c4ccc5sc6ccccc6c5c4)c4cccc5c6ccccc6n(-c6cccc(-c7cccc8ccccc78)c6)c45)cc3)cc2)cc1 ; c1ccc(-c2ccc(-c3ccc(N(c4cccc5c4sc4ccccc45)c4cccc5c6ccccc6n(-c6cccc(-c7cccc8ccccc78)c6)c45)cc3)cc2)cc1 ; c1ccc(-c2ccccc2N(c2cccc3oc4ccccc4c23)c2cccc3c4ccccc4n(-c4ccc(-c5cccc6ccccc56)cc4)c23)cc1]

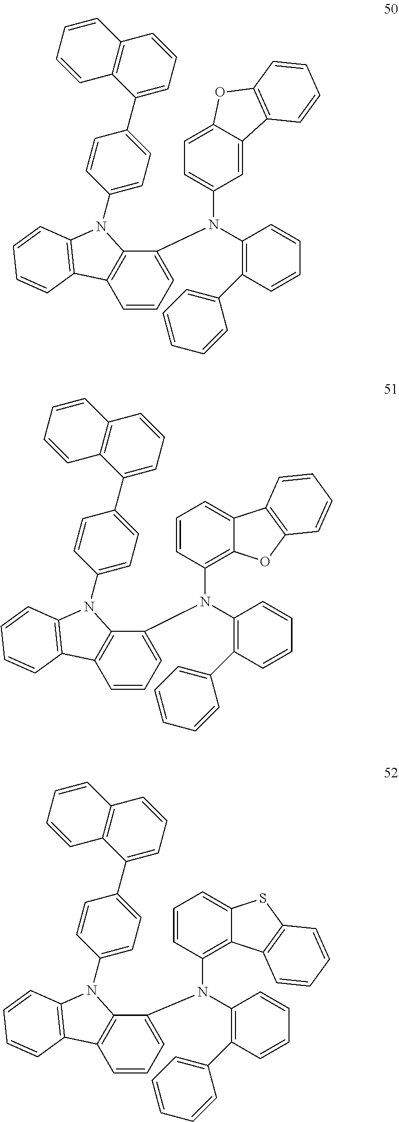 [SMILES: c1ccc(-c2ccccc2N(c2ccc3oc4ccccc4c3c2)c2cccc3c4ccccc4n(-c4ccc(-c5cccc6ccccc56)cc4)c23)cc1 ; c1ccc(-c2ccccc2N(c2cccc3c2oc2ccccc23)c2cccc3c4ccccc4n(-c4ccc(-c5cccc6ccccc56)cc4)c23)cc1 ; c1ccc(-c2ccccc2N(c2cccc3sc4ccccc4c23)c2cccc3c4ccccc4n(-c4ccc(-c5cccc6ccccc56)cc4)c23)cc1]

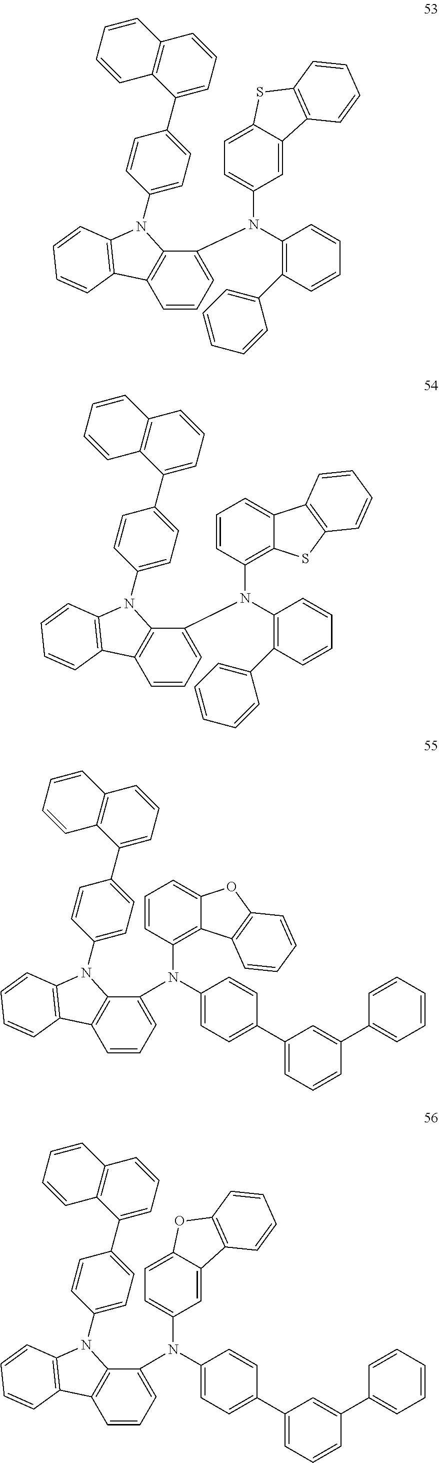 [SMILES: c1ccc(-c2ccccc2N(c2ccc3sc4ccccc4c3c2)c2cccc3c4ccccc4n(-c4ccc(-c5cccc6ccccc56)cc4)c23)cc1 ; c1ccc(-c2ccccc2N(c2cccc3c2sc2ccccc23)c2cccc3c4ccccc4n(-c4ccc(-c5cccc6ccccc56)cc4)c23)cc1 ; c1ccc(-c2cccc(-c3ccc(N(c4cccc5oc6ccccc6c45)c4cccc5c6ccccc6n(-c6ccc(-c7cccc8ccccc78)cc6)c45)cc3)c2)cc1 ; c1ccc(-c2cccc(-c3ccc(N(c4ccc5oc6ccccc6c5c4)c4cccc5c6ccccc6n(-c6ccc(-c7cccc8ccccc78)cc6)c45)cc3)c2)cc1]

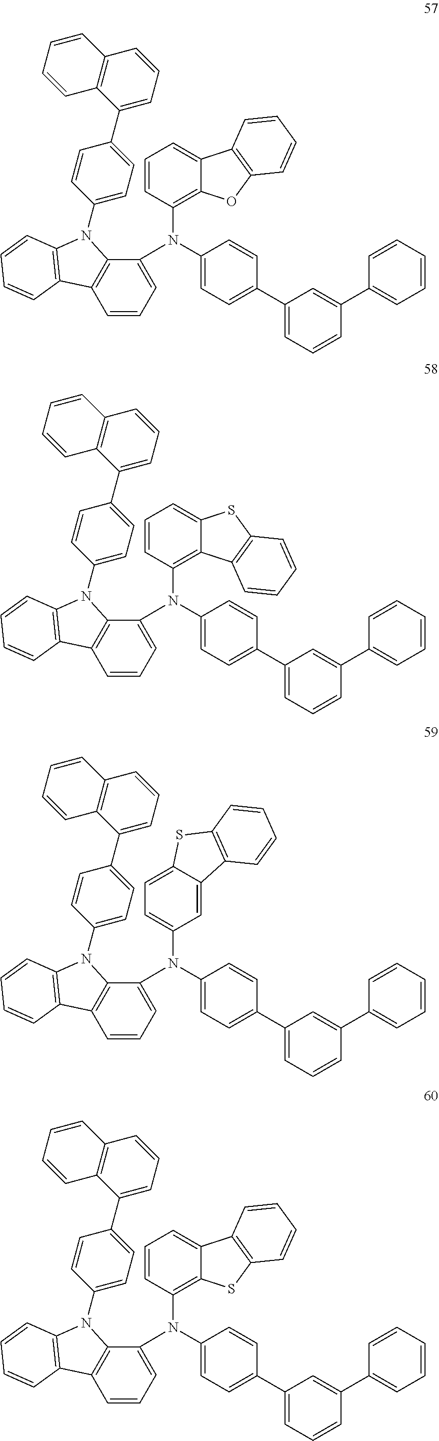 [SMILES: c1ccc(-c2cccc(-c3ccc(N(c4cccc5c4oc4ccccc45)c4cccc5c6ccccc6n(-c6ccc(-c7cccc8ccccc78)cc6)c45)cc3)c2)cc1 ; c1ccc(-c2cccc(-c3ccc(N(c4cccc5sc6ccccc6c45)c4cccc5c6ccccc6n(-c6ccc(-c7cccc8ccccc78)cc6)c45)cc3)c2)cc1 ; c1ccc(-c2cccc(-c3ccc(N(c4ccc5sc6ccccc6c5c4)c4cccc5c6ccccc6n(-c6ccc(-c7cccc8ccccc78)cc6)c45)cc3)c2)cc1 ; c1ccc(-c2cccc(-c3ccc(N(c4cccc5c4sc4ccccc45)c4cccc5c6ccccc6n(-c6ccc(-c7cccc8ccccc78)cc6)c45)cc3)c2)cc1]

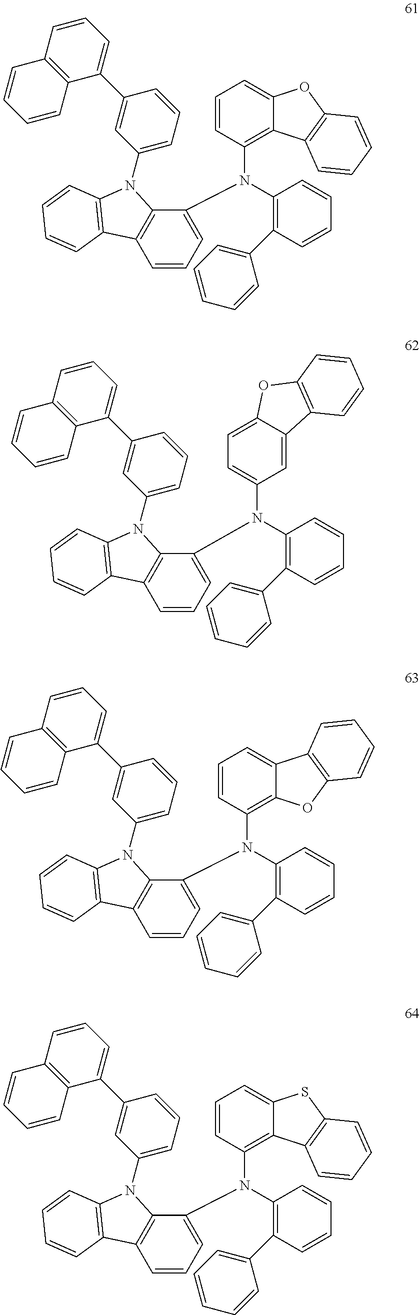 [SMILES: c1ccc(-c2ccccc2N(c2cccc3oc4ccccc4c23)c2cccc3c4ccccc4n(-c4cccc(-c5cccc6ccccc56)c4)c23)cc1 ; c1ccc(-c2ccccc2N(c2ccc3oc4ccccc4c3c2)c2cccc3c4ccccc4n(-c4cccc(-c5cccc6ccccc56)c4)c23)cc1 ; c1ccc(-c2ccccc2N(c2cccc3c2oc2ccccc23)c2cccc3c4ccccc4n(-c4cccc(-c5cccc6ccccc56)c4)c23)cc1 ; c1ccc(-c2ccccc2N(c2cccc3sc4ccccc4c23)c2cccc3c4ccccc4n(-c4cccc(-c5cccc6ccccc56)c4)c23)cc1]

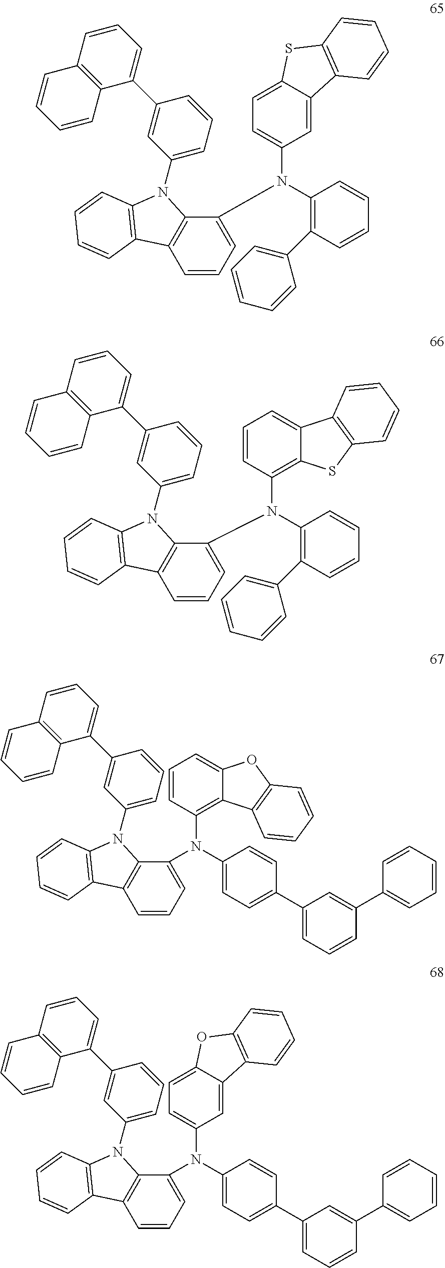 [SMILES: c1ccc(-c2ccccc2N(c2ccc3sc4ccccc4c3c2)c2cccc3c4ccccc4n(-c4cccc(-c5cccc6ccccc56)c4)c23)cc1 ; c1ccc(-c2ccccc2N(c2cccc3c2sc2ccccc23)c2cccc3c4ccccc4n(-c4cccc(-c5cccc6ccccc56)c4)c23)cc1 ; c1ccc(-c2cccc(-c3ccc(N(c4cccc5oc6ccccc6c45)c4cccc5c6ccccc6n(-c6cccc(-c7cccc8ccccc78)c6)c45)cc3)c2)cc1 ; c1ccc(-c2cccc(-c3ccc(N(c4ccc5oc6ccccc6c5c4)c4cccc5c6ccccc6n(-c6cccc(-c7cccc8ccccc78)c6)c45)cc3)c2)cc1]

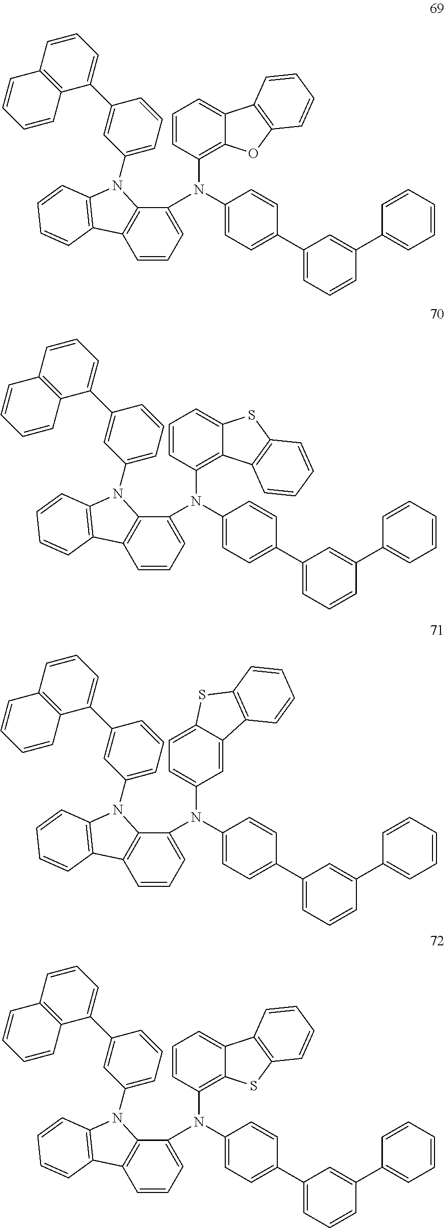 [SMILES: c1ccc(-c2cccc(-c3ccc(N(c4cccc5c4oc4ccccc45)c4cccc5c6ccccc6n(-c6cccc(-c7cccc8ccccc78)c6)c45)cc3)c2)cc1 ; c1ccc(-c2cccc(-c3ccc(N(c4cccc5sc6ccccc6c45)c4cccc5c6ccccc6n(-c6cccc(-c7cccc8ccccc78)c6)c45)cc3)c2)cc1 ; c1ccc(-c2cccc(-c3ccc(N(c4ccc5sc6ccccc6c5c4)c4cccc5c6ccccc6n(-c6cccc(-c7cccc8ccccc78)c6)c45)cc3)c2)cc1 ; c1ccc(-c2cccc(-c3ccc(N(c4cccc5c4sc4ccccc45)c4cccc5c6ccccc6n(-c6cccc(-c7cccc8ccccc78)c6)c45)cc3)c2)cc1]

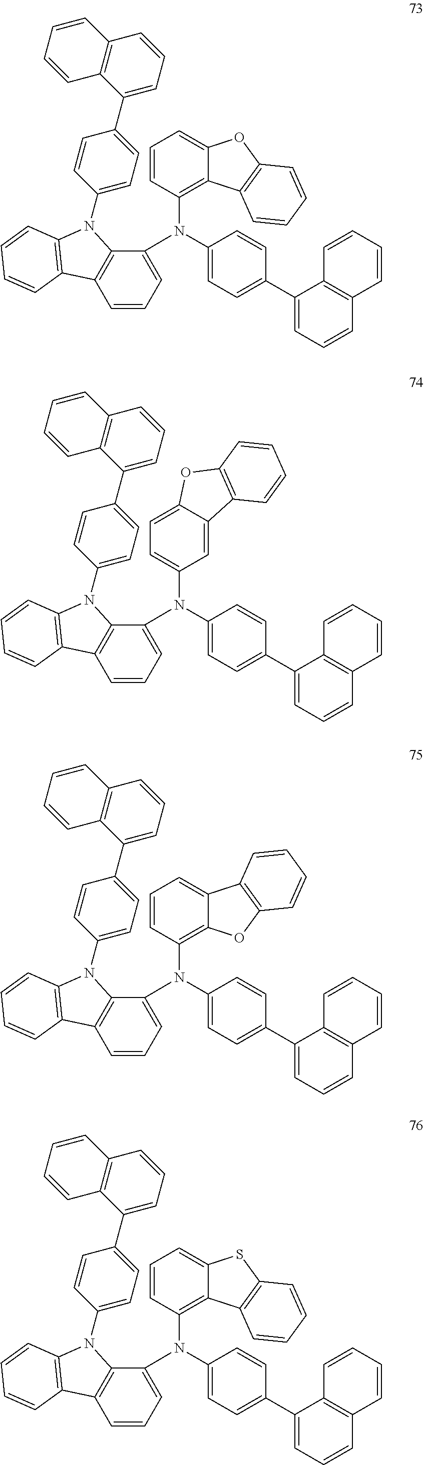 [SMILES: c1ccc2c(-c3ccc(N(c4cccc5oc6ccccc6c45)c4cccc5c6ccccc6n(-c6ccc(-c7cccc8ccccc78)cc6)c45)cc3)cccc2c1 ; c1ccc2c(-c3ccc(N(c4ccc5oc6ccccc6c5c4)c4cccc5c6ccccc6n(-c6ccc(-c7cccc8ccccc78)cc6)c45)cc3)cccc2c1 ; c1ccc2c(-c3ccc(N(c4cccc5c4oc4ccccc45)c4cccc5c6ccccc6n(-c6ccc(-c7cccc8ccccc78)cc6)c45)cc3)cccc2c1 ; c1ccc2c(-c3ccc(N(c4cccc5sc6ccccc6c45)c4cccc5c6ccccc6n(-c6ccc(-c7cccc8ccccc78)cc6)c45)cc3)cccc2c1]

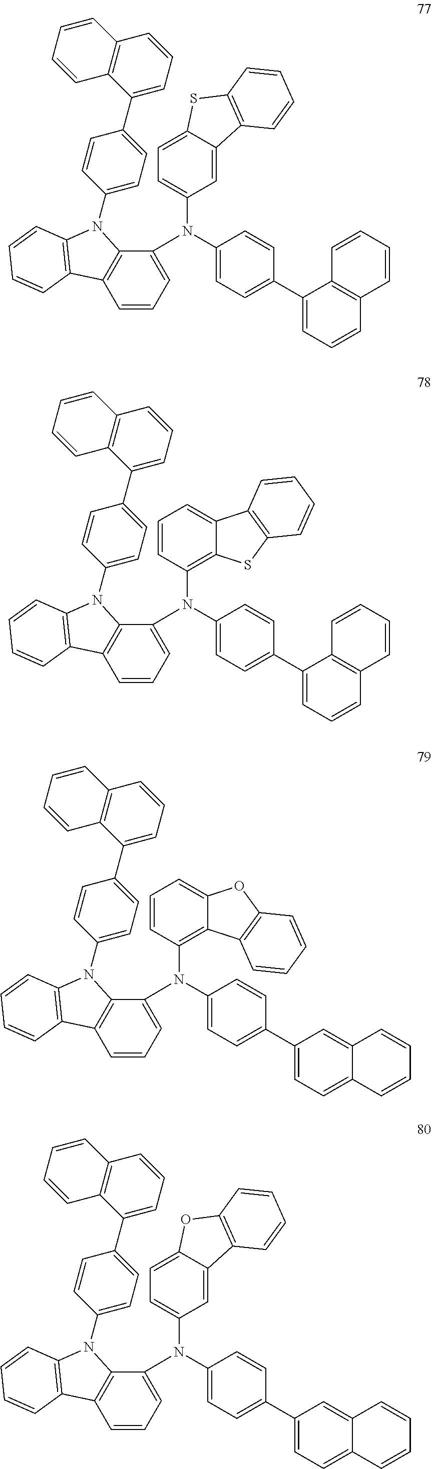 [SMILES: c1ccc2c(-c3ccc(N(c4ccc5sc6ccccc6c5c4)c4cccc5c6ccccc6n(-c6ccc(-c7cccc8ccccc78)cc6)c45)cc3)cccc2c1 ; c1ccc2c(-c3ccc(N(c4cccc5c4sc4ccccc45)c4cccc5c6ccccc6n(-c6ccc(-c7cccc8ccccc78)cc6)c45)cc3)cccc2c1 ; c1ccc2cc(-c3ccc(N(c4cccc5oc6ccccc6c45)c4cccc5c6ccccc6n(-c6ccc(-c7cccc8ccccc78)cc6)c45)cc3)ccc2c1 ; c1ccc2cc(-c3ccc(N(c4ccc5oc6ccccc6c5c4)c4cccc5c6ccccc6n(-c6ccc(-c7cccc8ccccc78)cc6)c45)cc3)ccc2c1]

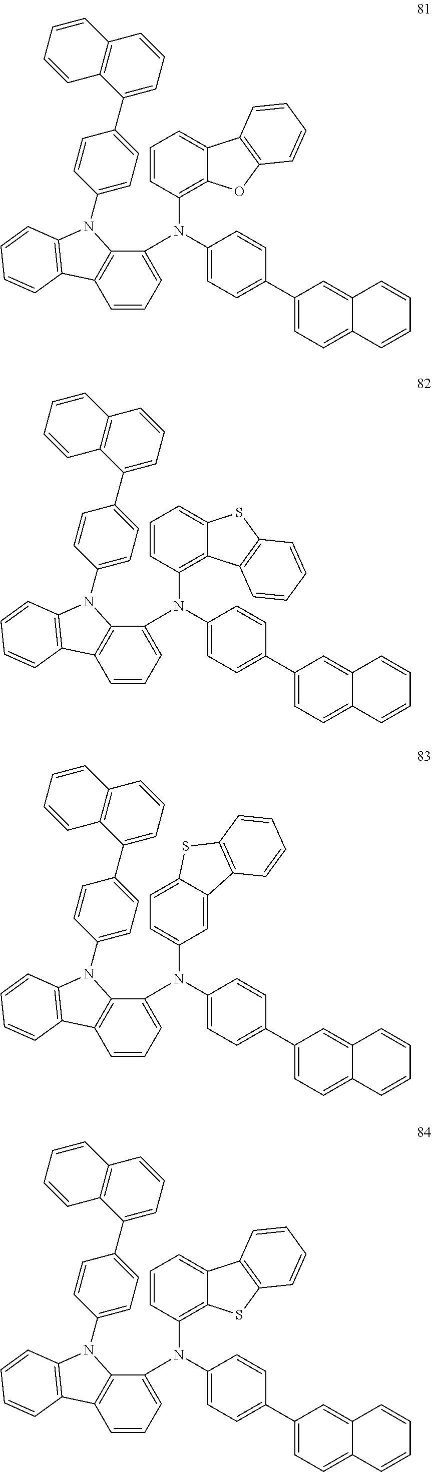 [SMILES: c1ccc2cc(-c3ccc(N(c4cccc5c4oc4ccccc45)c4cccc5c6ccccc6n(-c6ccc(-c7cccc8ccccc78)cc6)c45)cc3)ccc2c1 ; c1ccc2cc(-c3ccc(N(c4cccc5sc6ccccc6c45)c4cccc5c6ccccc6n(-c6ccc(-c7cccc8ccccc78)cc6)c45)cc3)ccc2c1 ; c1ccc2cc(-c3ccc(N(c4ccc5sc6ccccc6c5c4)c4cccc5c6ccccc6n(-c6ccc(-c7cccc8ccccc78)cc6)c45)cc3)ccc2c1 ; c1ccc2cc(-c3ccc(N(c4cccc5c4sc4ccccc45)c4cccc5c6ccccc6n(-c6ccc(-c7cccc8ccccc78)cc6)c45)cc3)ccc2c1]

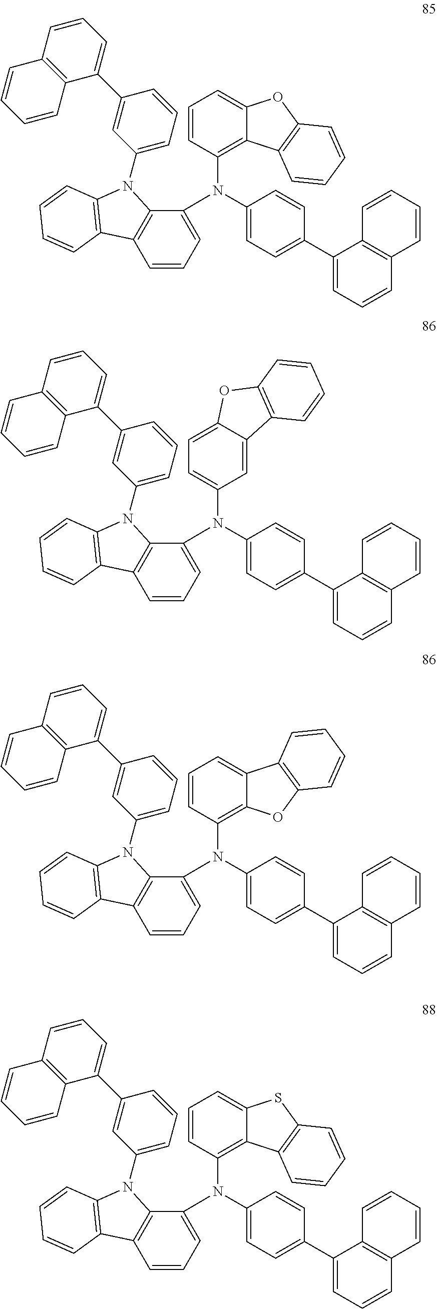 [SMILES: c1cc(-c2cccc3ccccc23)cc(-n2c3ccccc3c3cccc(N(c4ccc(-c5cccc6ccccc56)cc4)c4cccc5oc6ccccc6c45)c32)c1 ; c1cc(-c2cccc3ccccc23)cc(-n2c3ccccc3c3cccc(N(c4ccc(-c5cccc6ccccc56)cc4)c4ccc5oc6ccccc6c5c4)c32)c1 ; c1cc(-c2cccc3ccccc23)cc(-n2c3ccccc3c3cccc(N(c4ccc(-c5cccc6ccccc56)cc4)c4cccc5c4oc4ccccc45)c32)c1 ; c1cc(-c2cccc3ccccc23)cc(-n2c3ccccc3c3cccc(N(c4ccc(-c5cccc6ccccc56)cc4)c4cccc5sc6ccccc6c45)c32)c1]

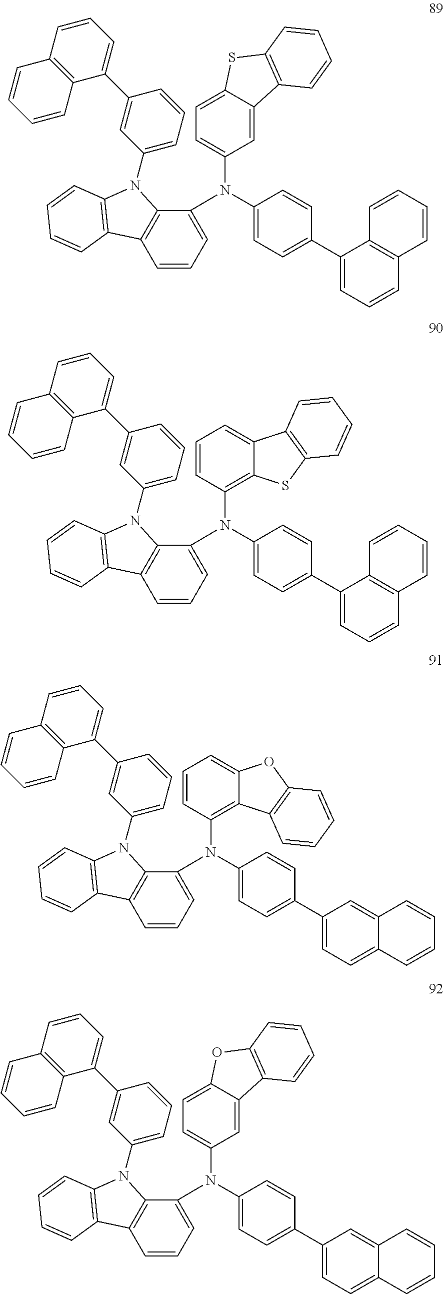 [SMILES: c1cc(-c2cccc3ccccc23)cc(-n2c3ccccc3c3cccc(N(c4ccc(-c5cccc6ccccc56)cc4)c4ccc5sc6ccccc6c5c4)c32)c1 ; c1cc(-c2cccc3ccccc23)cc(-n2c3ccccc3c3cccc(N(c4ccc(-c5cccc6ccccc56)cc4)c4cccc5c4sc4ccccc45)c32)c1 ; c1cc(-c2cccc3ccccc23)cc(-n2c3ccccc3c3cccc(N(c4ccc(-c5ccc6ccccc6c5)cc4)c4cccc5oc6ccccc6c45)c32)c1 ; c1cc(-c2cccc3ccccc23)cc(-n2c3ccccc3c3cccc(N(c4ccc(-c5ccc6ccccc6c5)cc4)c4ccc5oc6ccccc6c5c4)c32)c1]

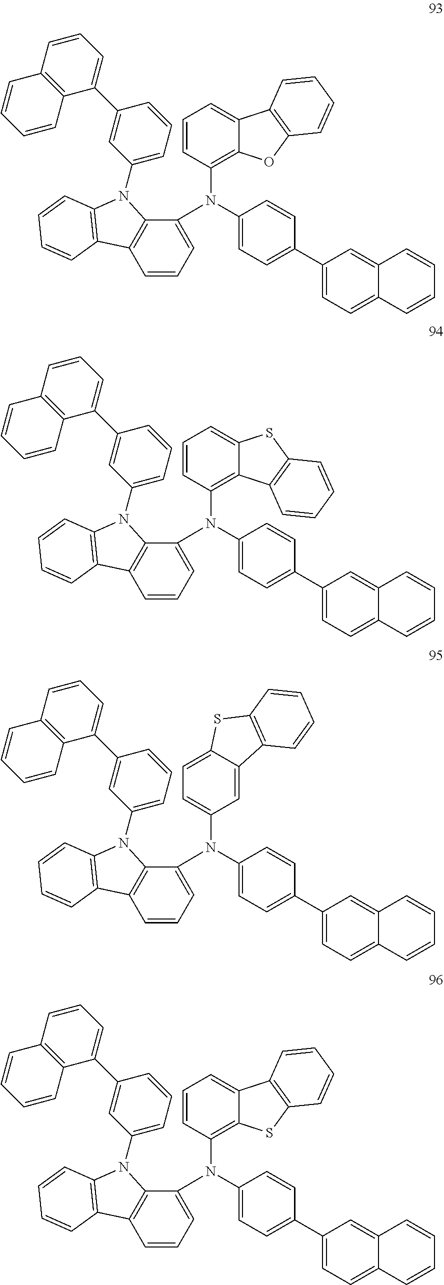 [SMILES: c1cc(-c2cccc3ccccc23)cc(-n2c3ccccc3c3cccc(N(c4ccc(-c5ccc6ccccc6c5)cc4)c4cccc5c4oc4ccccc45)c32)c1 ; c1cc(-c2cccc3ccccc23)cc(-n2c3ccccc3c3cccc(N(c4ccc(-c5ccc6ccccc6c5)cc4)c4cccc5sc6ccccc6c45)c32)c1 ; c1cc(-c2cccc3ccccc23)cc(-n2c3ccccc3c3cccc(N(c4ccc(-c5ccc6ccccc6c5)cc4)c4ccc5sc6ccccc6c5c4)c32)c1 ; c1cc(-c2cccc3ccccc23)cc(-n2c3ccccc3c3cccc(N(c4ccc(-c5ccc6ccccc6c5)cc4)c4cccc5c4sc4ccccc45)c32)c1]

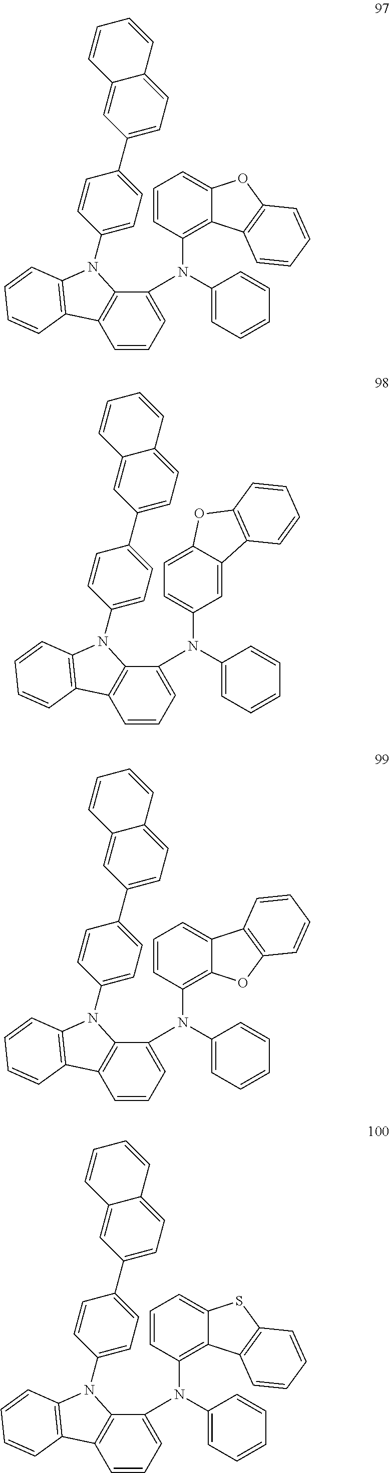

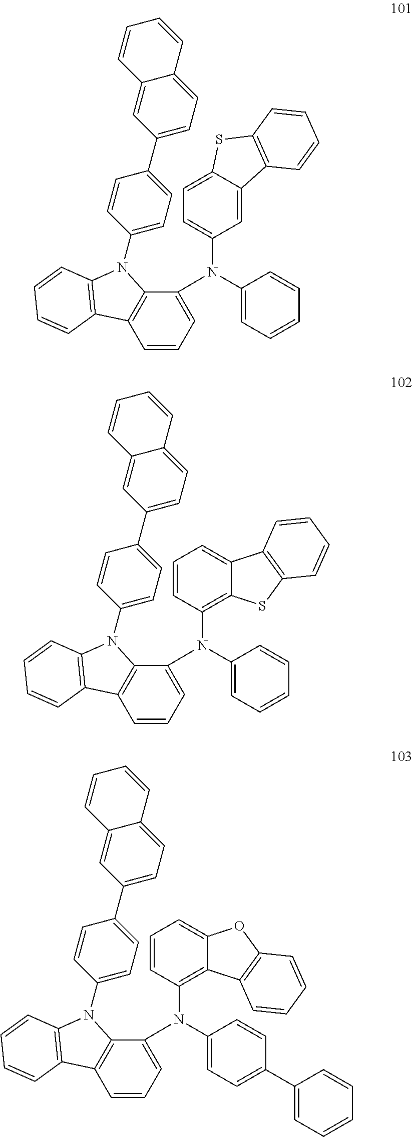

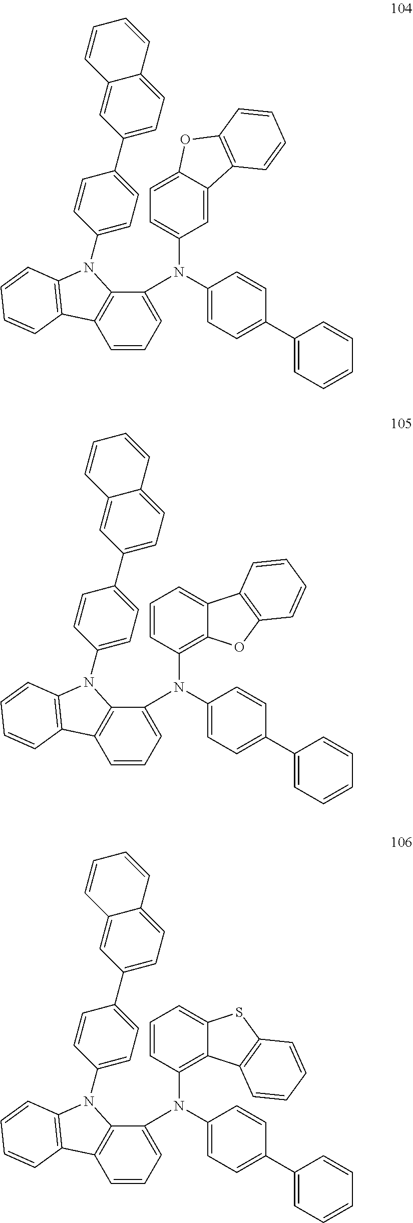 [SMILES: c1ccc(-c2ccc(N(c3ccc4oc5ccccc5c4c3)c3cccc4c5ccccc5n(-c5ccc(-c6ccc7ccccc7c6)cc5)c34)cc2)cc1 ; c1ccc(-c2ccc(N(c3cccc4c3oc3ccccc34)c3cccc4c5ccccc5n(-c5ccc(-c6ccc7ccccc7c6)cc5)c34)cc2)cc1 ; c1ccc(-c2ccc(N(c3cccc4sc5ccccc5c34)c3cccc4c5ccccc5n(-c5ccc(-c6ccc7ccccc7c6)cc5)c34)cc2)cc1]

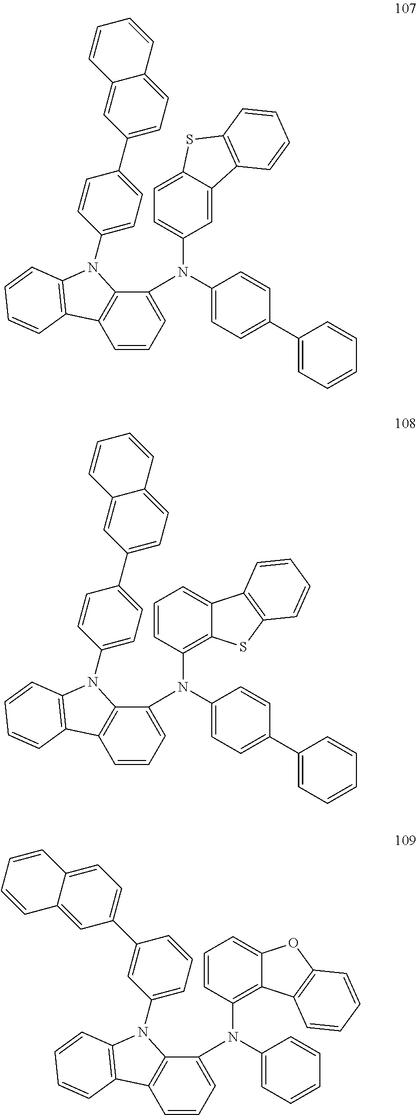 [SMILES: c1ccc(-c2ccc(N(c3ccc4sc5ccccc5c4c3)c3cccc4c5ccccc5n(-c5ccc(-c6ccc7ccccc7c6)cc5)c34)cc2)cc1 ; c1ccc(-c2ccc(N(c3cccc4c3sc3ccccc34)c3cccc4c5ccccc5n(-c5ccc(-c6ccc7ccccc7c6)cc5)c34)cc2)cc1 ; c1ccc(N(c2cccc3oc4ccccc4c23)c2cccc3c4ccccc4n(-c4cccc(-c5ccc6ccccc6c5)c4)c23)cc1]

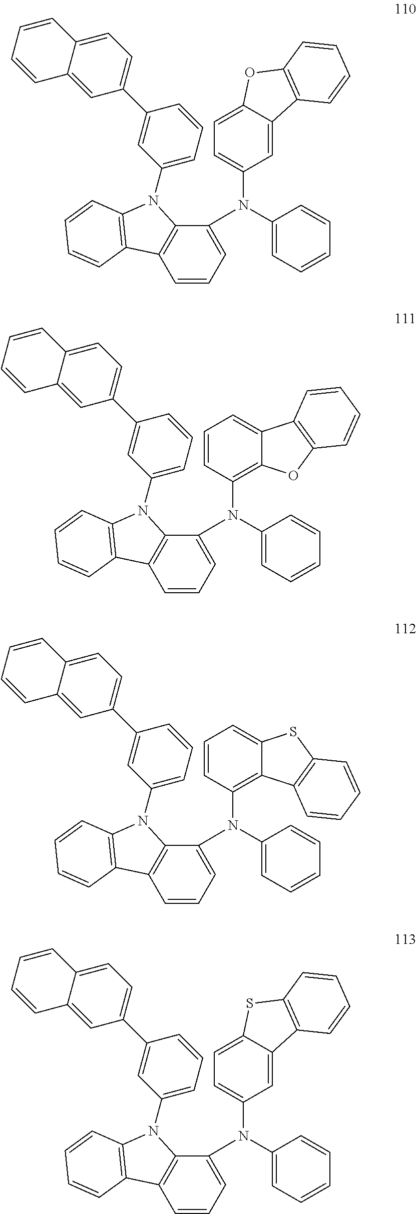 [SMILES: c1ccc(N(c2ccc3oc4ccccc4c3c2)c2cccc3c4ccccc4n(-c4cccc(-c5ccc6ccccc6c5)c4)c23)cc1 ; c1ccc(N(c2cccc3c2oc2ccccc23)c2cccc3c4ccccc4n(-c4cccc(-c5ccc6ccccc6c5)c4)c23)cc1 ; c1ccc(N(c2cccc3sc4ccccc4c23)c2cccc3c4ccccc4n(-c4cccc(-c5ccc6ccccc6c5)c4)c23)cc1 ; c1ccc(N(c2ccc3sc4ccccc4c3c2)c2cccc3c4ccccc4n(-c4cccc(-c5ccc6ccccc6c5)c4)c23)cc1]

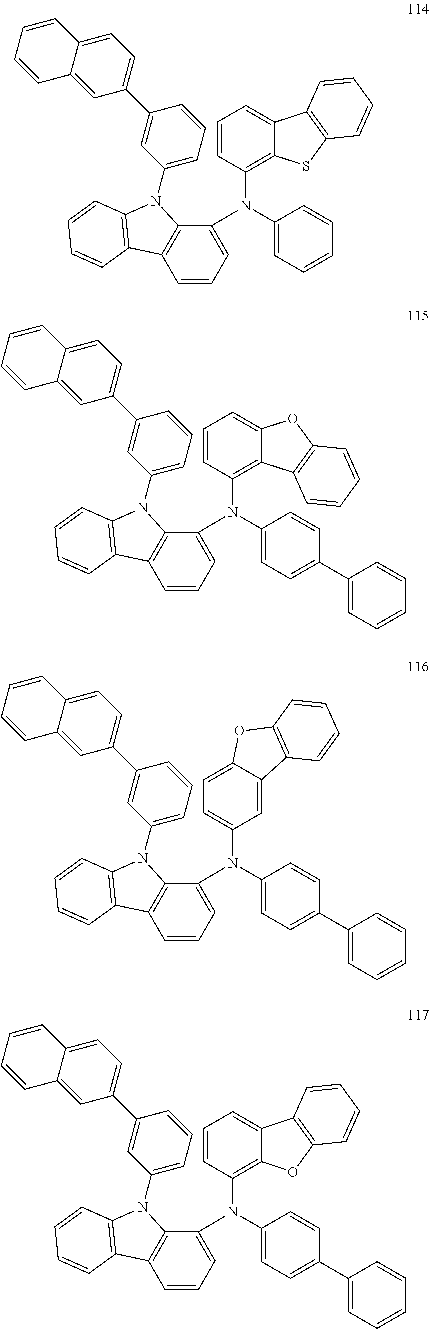 [SMILES: c1ccc(N(c2cccc3c2sc2ccccc23)c2cccc3c4ccccc4n(-c4cccc(-c5ccc6ccccc6c5)c4)c23)cc1 ; c1ccc(-c2ccc(N(c3cccc4oc5ccccc5c34)c3cccc4c5ccccc5n(-c5cccc(-c6ccc7ccccc7c6)c5)c34)cc2)cc1 ; c1ccc(-c2ccc(N(c3ccc4oc5ccccc5c4c3)c3cccc4c5ccccc5n(-c5cccc(-c6ccc7ccccc7c6)c5)c34)cc2)cc1 ; c1ccc(-c2ccc(N(c3cccc4c3oc3ccccc34)c3cccc4c5ccccc5n(-c5cccc(-c6ccc7ccccc7c6)c5)c34)cc2)cc1]

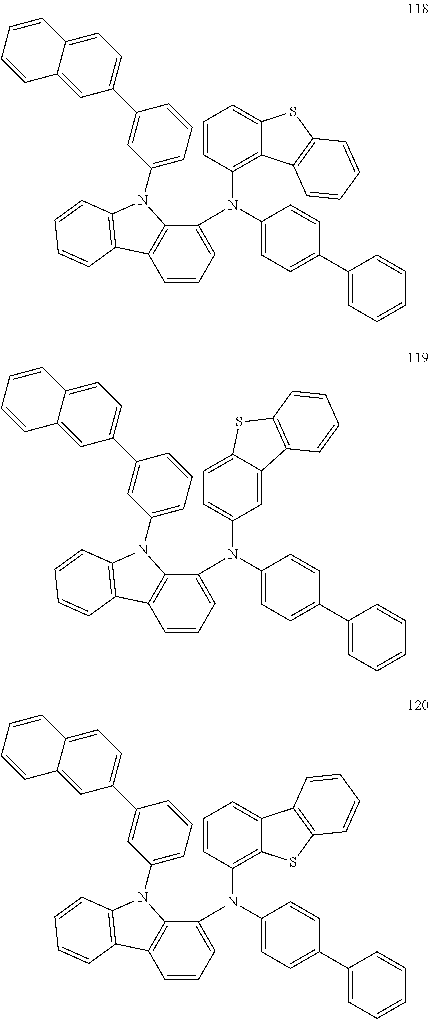 [SMILES: c1ccc(-c2ccc(N(c3cccc4sc5ccccc5c34)c3cccc4c5ccccc5n(-c5cccc(-c6ccc7ccccc7c6)c5)c34)cc2)cc1 ; c1ccc(-c2ccc(N(c3ccc4sc5ccccc5c4c3)c3cccc4c5ccccc5n(-c5cccc(-c6ccc7ccccc7c6)c5)c34)cc2)cc1 ; c1ccc(-c2ccc(N(c3cccc4c3sc3ccccc34)c3cccc4c5ccccc5n(-c5cccc(-c6ccc7ccccc7c6)c5)c34)cc2)cc1]

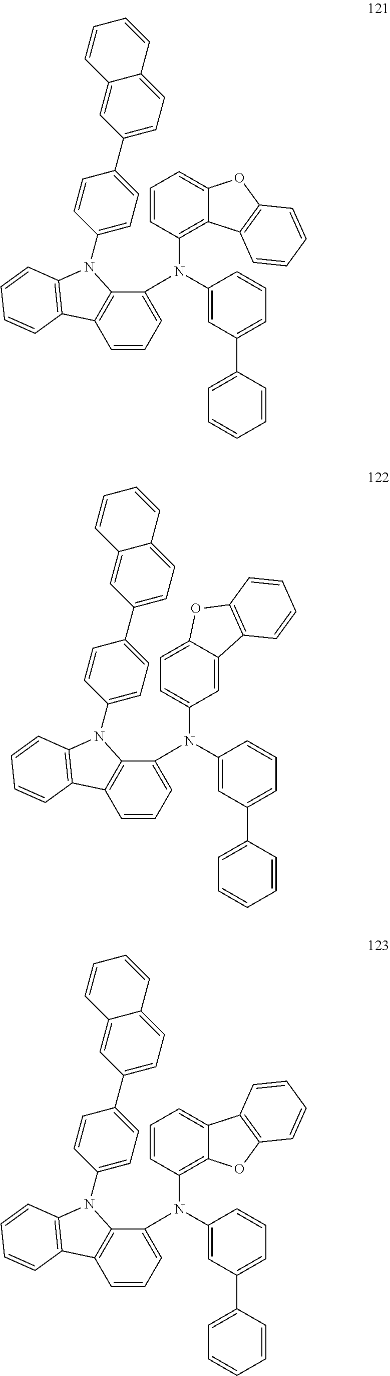 [SMILES: c1ccc(-c2cccc(N(c3cccc4oc5ccccc5c34)c3cccc4c5ccccc5n(-c5ccc(-c6ccc7ccccc7c6)cc5)c34)c2)cc1 ; c1ccc(-c2cccc(N(c3ccc4oc5ccccc5c4c3)c3cccc4c5ccccc5n(-c5ccc(-c6ccc7ccccc7c6)cc5)c34)c2)cc1 ; c1ccc(-c2cccc(N(c3cccc4c3oc3ccccc34)c3cccc4c5ccccc5n(-c5ccc(-c6ccc7ccccc7c6)cc5)c34)c2)cc1]

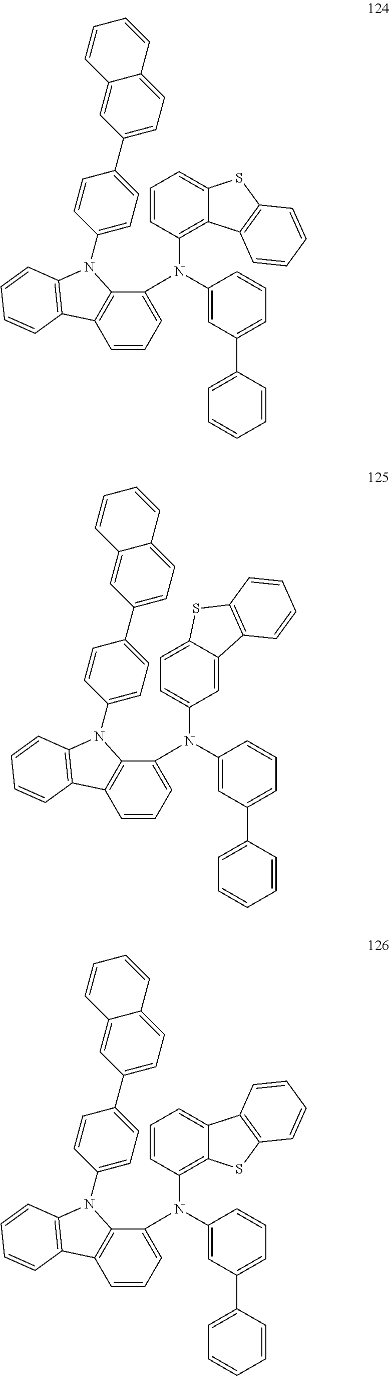 [SMILES: c1ccc(-c2cccc(N(c3cccc4sc5ccccc5c34)c3cccc4c5ccccc5n(-c5ccc(-c6ccc7ccccc7c6)cc5)c34)c2)cc1 ; c1ccc(-c2cccc(N(c3ccc4sc5ccccc5c4c3)c3cccc4c5ccccc5n(-c5ccc(-c6ccc7ccccc7c6)cc5)c34)c2)cc1 ; c1ccc(-c2cccc(N(c3cccc4c3sc3ccccc34)c3cccc4c5ccccc5n(-c5ccc(-c6ccc7ccccc7c6)cc5)c34)c2)cc1]

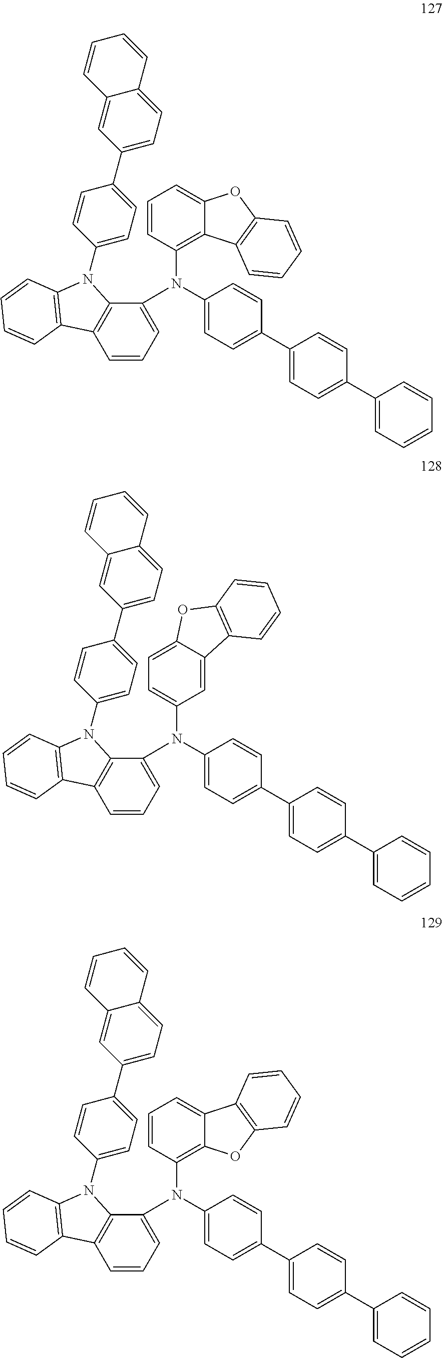 [SMILES: c1ccc(-c2ccc(-c3ccc(N(c4cccc5oc6ccccc6c45)c4cccc5c6ccccc6n(-c6ccc(-c7ccc8ccccc8c7)cc6)c45)cc3)cc2)cc1 ; c1ccc(-c2ccc(-c3ccc(N(c4ccc5oc6ccccc6c5c4)c4cccc5c6ccccc6n(-c6ccc(-c7ccc8ccccc8c7)cc6)c45)cc3)cc2)cc1 ; c1ccc(-c2ccc(-c3ccc(N(c4cccc5c4oc4ccccc45)c4cccc5c6ccccc6n(-c6ccc(-c7ccc8ccccc8c7)cc6)c45)cc3)cc2)cc1]

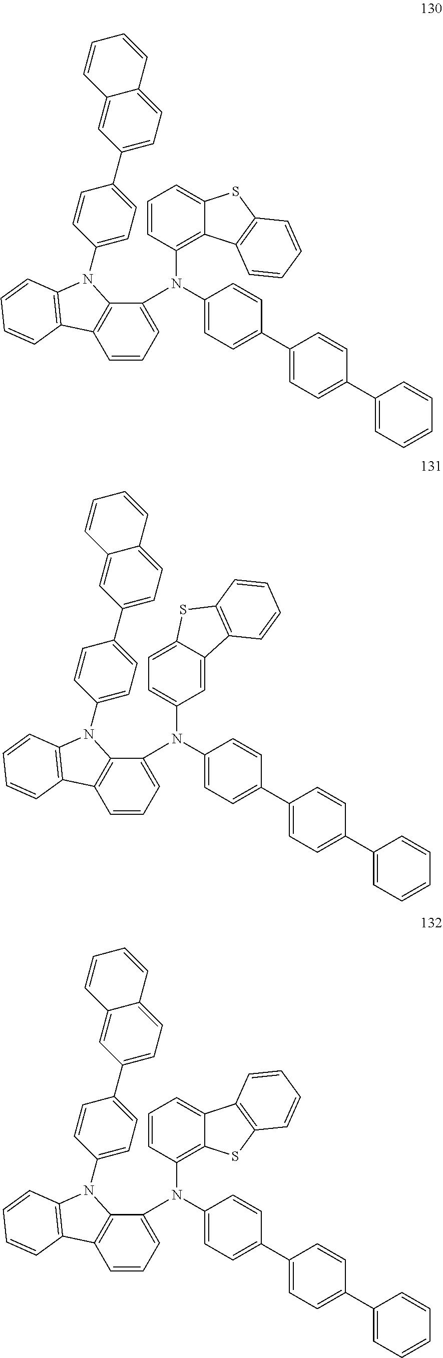 [SMILES: c1ccc(-c2ccc(-c3ccc(N(c4cccc5sc6ccccc6c45)c4cccc5c6ccccc6n(-c6ccc(-c7ccc8ccccc8c7)cc6)c45)cc3)cc2)cc1 ; c1ccc(-c2ccc(-c3ccc(N(c4ccc5sc6ccccc6c5c4)c4cccc5c6ccccc6n(-c6ccc(-c7ccc8ccccc8c7)cc6)c45)cc3)cc2)cc1 ; c1ccc(-c2ccc(-c3ccc(N(c4cccc5c4sc4ccccc45)c4cccc5c6ccccc6n(-c6ccc(-c7ccc8ccccc8c7)cc6)c45)cc3)cc2)cc1]

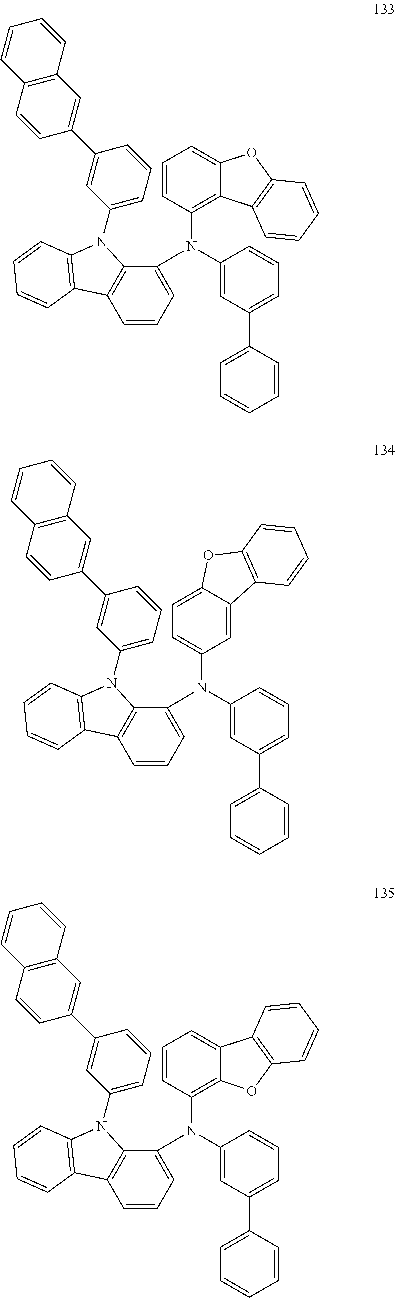 [SMILES: c1ccc(-c2cccc(N(c3cccc4oc5ccccc5c34)c3cccc4c5ccccc5n(-c5cccc(-c6ccc7ccccc7c6)c5)c34)c2)cc1 ; c1ccc(-c2cccc(N(c3ccc4oc5ccccc5c4c3)c3cccc4c5ccccc5n(-c5cccc(-c6ccc7ccccc7c6)c5)c34)c2)cc1 ; c1ccc(-c2cccc(N(c3cccc4c3oc3ccccc34)c3cccc4c5ccccc5n(-c5cccc(-c6ccc7ccccc7c6)c5)c34)c2)cc1]

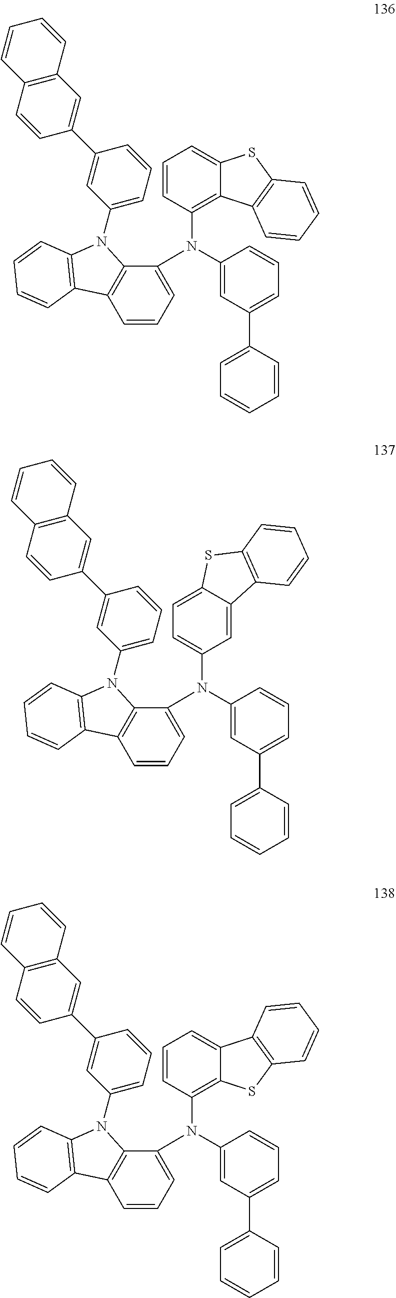 [SMILES: c1ccc(-c2cccc(N(c3cccc4sc5ccccc5c34)c3cccc4c5ccccc5n(-c5cccc(-c6ccc7ccccc7c6)c5)c34)c2)cc1 ; c1ccc(-c2cccc(N(c3ccc4sc5ccccc5c4c3)c3cccc4c5ccccc5n(-c5cccc(-c6ccc7ccccc7c6)c5)c34)c2)cc1 ; c1ccc(-c2cccc(N(c3cccc4c3sc3ccccc34)c3cccc4c5ccccc5n(-c5cccc(-c6ccc7ccccc7c6)c5)c34)c2)cc1]

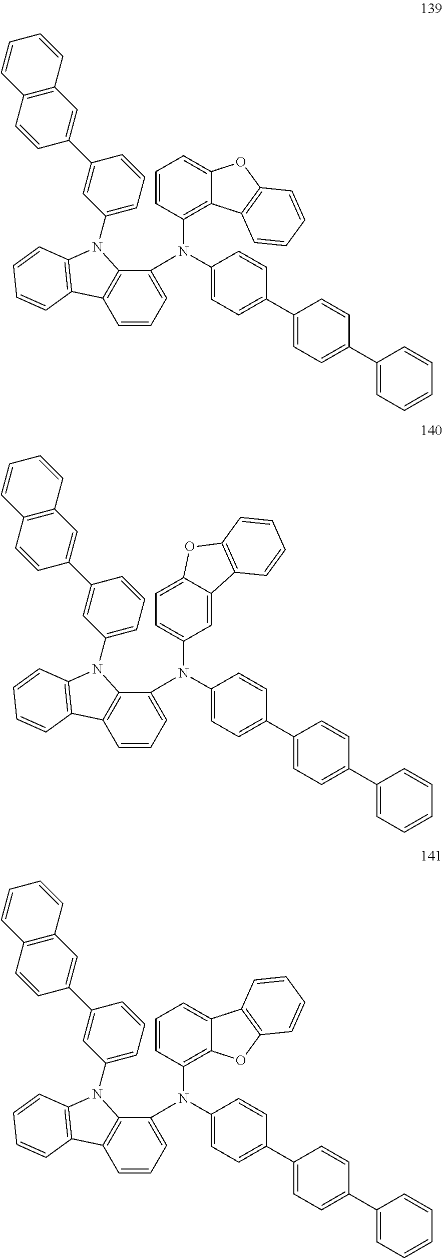 [SMILES: c1ccc(-c2ccc(-c3ccc(N(c4cccc5oc6ccccc6c45)c4cccc5c6ccccc6n(-c6cccc(-c7ccc8ccccc8c7)c6)c45)cc3)cc2)cc1 ; c1ccc(-c2ccc(-c3ccc(N(c4ccc5oc6ccccc6c5c4)c4cccc5c6ccccc6n(-c6cccc(-c7ccc8ccccc8c7)c6)c45)cc3)cc2)cc1 ; c1ccc(-c2ccc(-c3ccc(N(c4cccc5c4oc4ccccc45)c4cccc5c6ccccc6n(-c6cccc(-c7ccc8ccccc8c7)c6)c45)cc3)cc2)cc1]

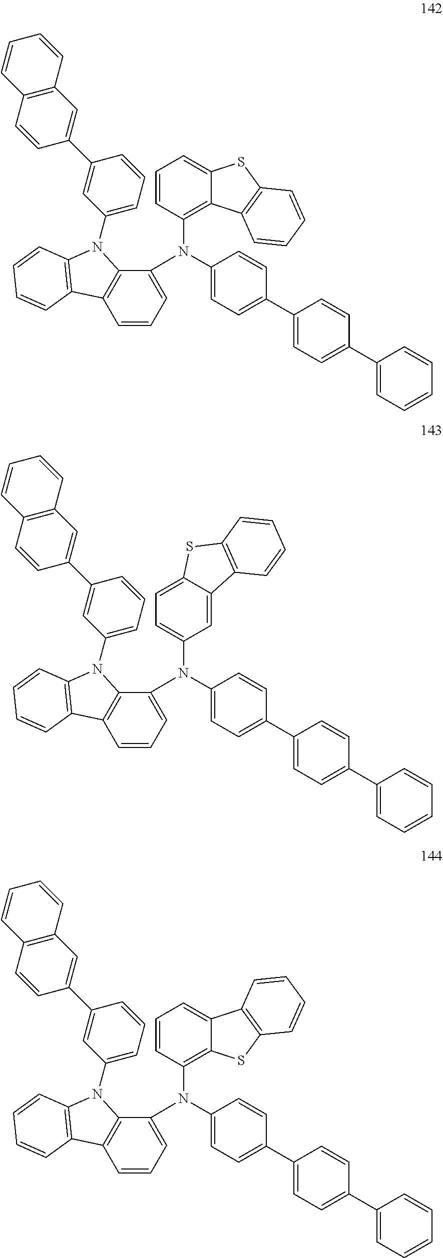 [SMILES: c1ccc(-c2ccc(-c3ccc(N(c4cccc5sc6ccccc6c45)c4cccc5c6ccccc6n(-c6cccc(-c7ccc8ccccc8c7)c6)c45)cc3)cc2)cc1 ; c1ccc(-c2ccc(-c3ccc(N(c4ccc5sc6ccccc6c5c4)c4cccc5c6ccccc6n(-c6cccc(-c7ccc8ccccc8c7)c6)c45)cc3)cc2)cc1 ; c1ccc(-c2ccc(-c3ccc(N(c4cccc5c4sc4ccccc45)c4cccc5c6ccccc6n(-c6cccc(-c7ccc8ccccc8c7)c6)c45)cc3)cc2)cc1]

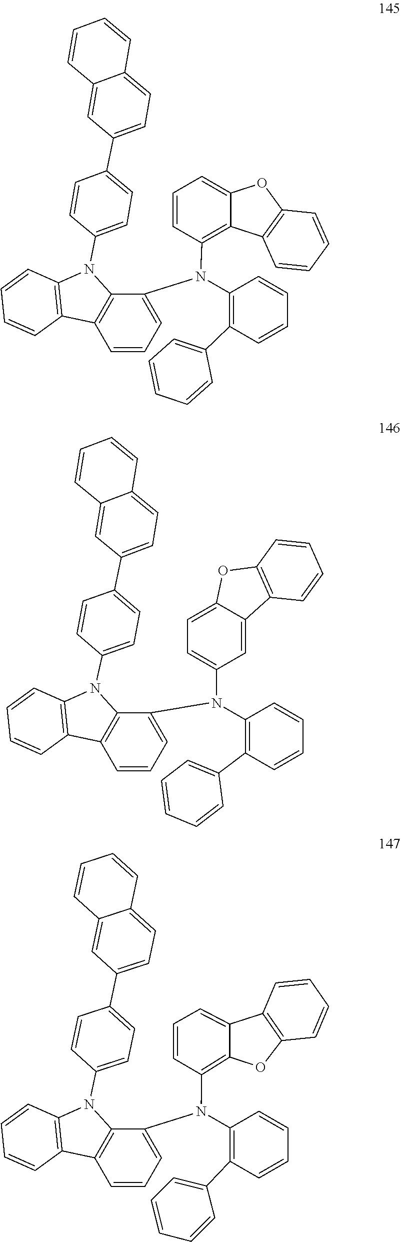 [SMILES: c1ccc(-c2ccccc2N(c2cccc3oc4ccccc4c23)c2cccc3c4ccccc4n(-c4ccc(-c5ccc6ccccc6c5)cc4)c23)cc1 ; c1ccc(-c2ccccc2N(c2ccc3oc4ccccc4c3c2)c2cccc3c4ccccc4n(-c4ccc(-c5ccc6ccccc6c5)cc4)c23)cc1 ; c1ccc(-c2ccccc2N(c2cccc3c2oc2ccccc23)c2cccc3c4ccccc4n(-c4ccc(-c5ccc6ccccc6c5)cc4)c23)cc1]

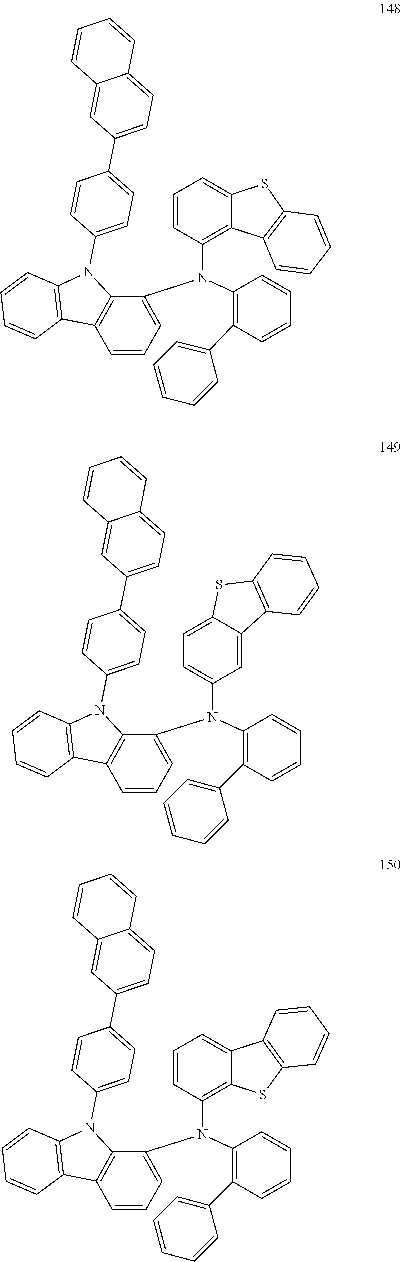 [SMILES: c1ccc(-c2ccccc2N(c2cccc3sc4ccccc4c23)c2cccc3c4ccccc4n(-c4ccc(-c5ccc6ccccc6c5)cc4)c23)cc1 ; c1ccc(-c2ccccc2N(c2ccc3sc4ccccc4c3c2)c2cccc3c4ccccc4n(-c4ccc(-c5ccc6ccccc6c5)cc4)c23)cc1 ; c1ccc(-c2ccccc2N(c2cccc3c2sc2ccccc23)c2cccc3c4ccccc4n(-c4ccc(-c5ccc6ccccc6c5)cc4)c23)cc1]

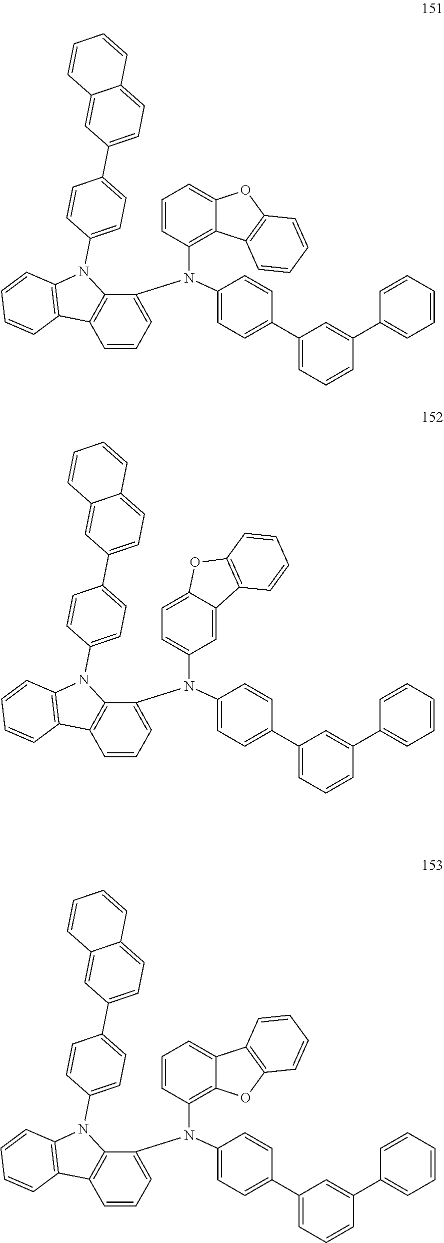 [SMILES: c1ccc(-c2cccc(-c3ccc(N(c4cccc5oc6ccccc6c45)c4cccc5c6ccccc6n(-c6ccc(-c7ccc8ccccc8c7)cc6)c45)cc3)c2)cc1 ; c1ccc(-c2cccc(-c3ccc(N(c4ccc5oc6ccccc6c5c4)c4cccc5c6ccccc6n(-c6ccc(-c7ccc8ccccc8c7)cc6)c45)cc3)c2)cc1 ; c1ccc(-c2cccc(-c3ccc(N(c4cccc5c4oc4ccccc45)c4cccc5c6ccccc6n(-c6ccc(-c7ccc8ccccc8c7)cc6)c45)cc3)c2)cc1]

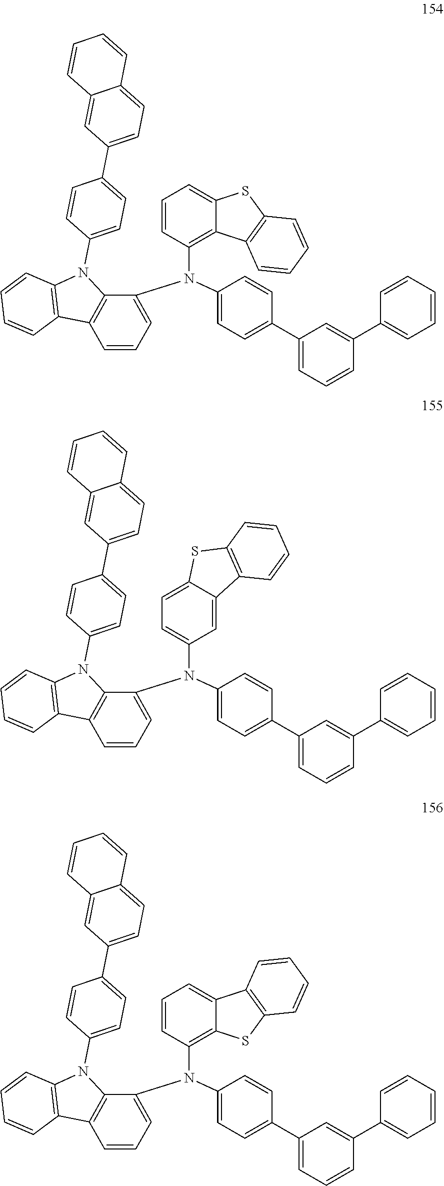 [SMILES: c1ccc(-c2cccc(-c3ccc(N(c4cccc5sc6ccccc6c45)c4cccc5c6ccccc6n(-c6ccc(-c7ccc8ccccc8c7)cc6)c45)cc3)c2)cc1 ; c1ccc(-c2cccc(-c3ccc(N(c4ccc5sc6ccccc6c5c4)c4cccc5c6ccccc6n(-c6ccc(-c7ccc8ccccc8c7)cc6)c45)cc3)c2)cc1 ; c1ccc(-c2cccc(-c3ccc(N(c4cccc5c4sc4ccccc45)c4cccc5c6ccccc6n(-c6ccc(-c7ccc8ccccc8c7)cc6)c45)cc3)c2)cc1]

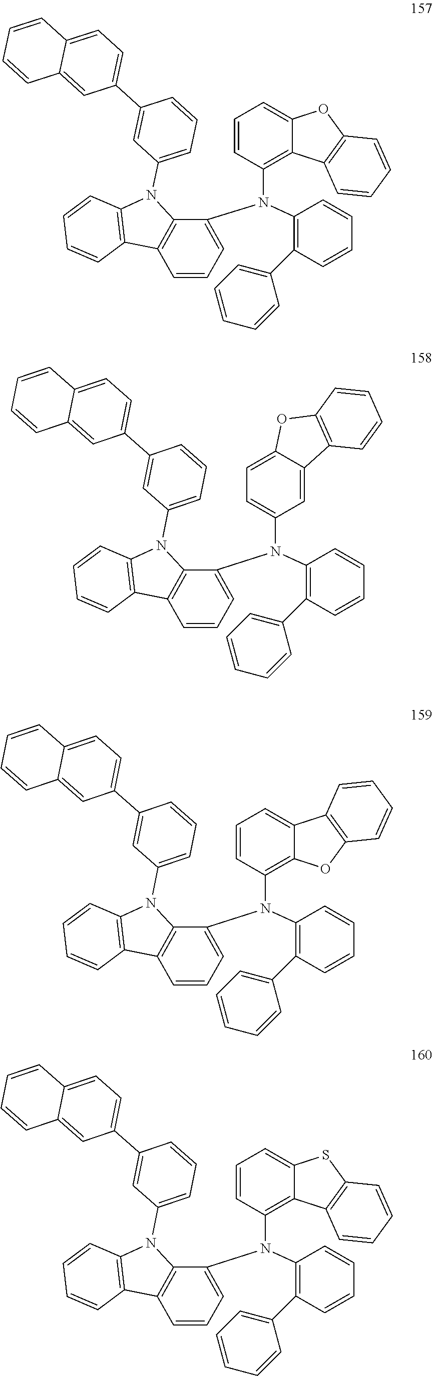 [SMILES: c1ccc(-c2ccccc2N(c2cccc3oc4ccccc4c23)c2cccc3c4ccccc4n(-c4cccc(-c5ccc6ccccc6c5)c4)c23)cc1 ; c1ccc(-c2ccccc2N(c2ccc3oc4ccccc4c3c2)c2cccc3c4ccccc4n(-c4cccc(-c5ccc6ccccc6c5)c4)c23)cc1 ; c1ccc(-c2ccccc2N(c2cccc3c2oc2ccccc23)c2cccc3c4ccccc4n(-c4cccc(-c5ccc6ccccc6c5)c4)c23)cc1 ; c1ccc(-c2ccccc2N(c2cccc3sc4ccccc4c23)c2cccc3c4ccccc4n(-c4cccc(-c5ccc6ccccc6c5)c4)c23)cc1]

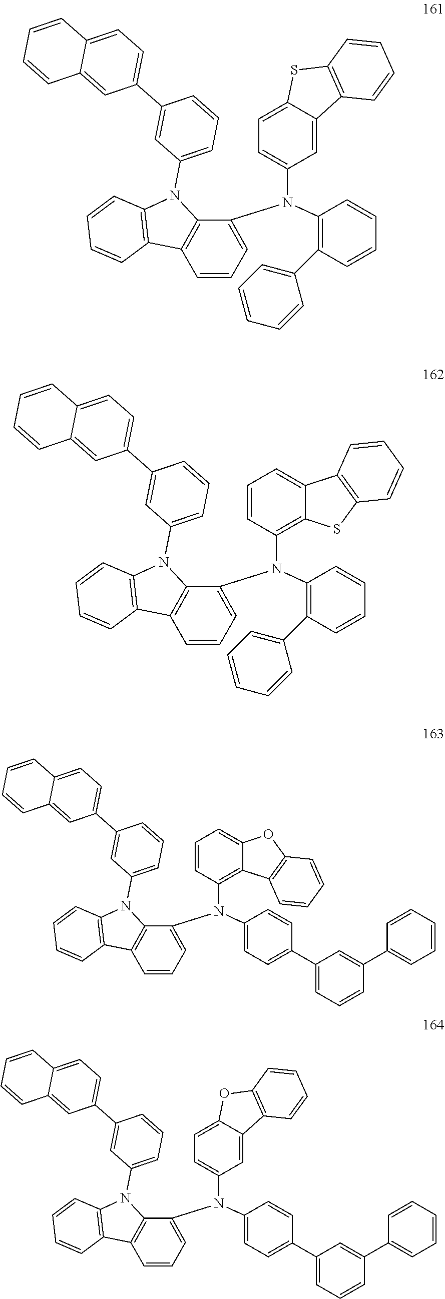 [SMILES: c1ccc(-c2ccccc2N(c2ccc3sc4ccccc4c3c2)c2cccc3c4ccccc4n(-c4cccc(-c5ccc6ccccc6c5)c4)c23)cc1 ; c1ccc(-c2ccccc2N(c2cccc3c2sc2ccccc23)c2cccc3c4ccccc4n(-c4cccc(-c5ccc6ccccc6c5)c4)c23)cc1 ; c1ccc(-c2cccc(-c3ccc(N(c4cccc5oc6ccccc6c45)c4cccc5c6ccccc6n(-c6cccc(-c7ccc8ccccc8c7)c6)c45)cc3)c2)cc1 ; c1ccc(-c2cccc(-c3ccc(N(c4ccc5oc6ccccc6c5c4)c4cccc5c6ccccc6n(-c6cccc(-c7ccc8ccccc8c7)c6)c45)cc3)c2)cc1]

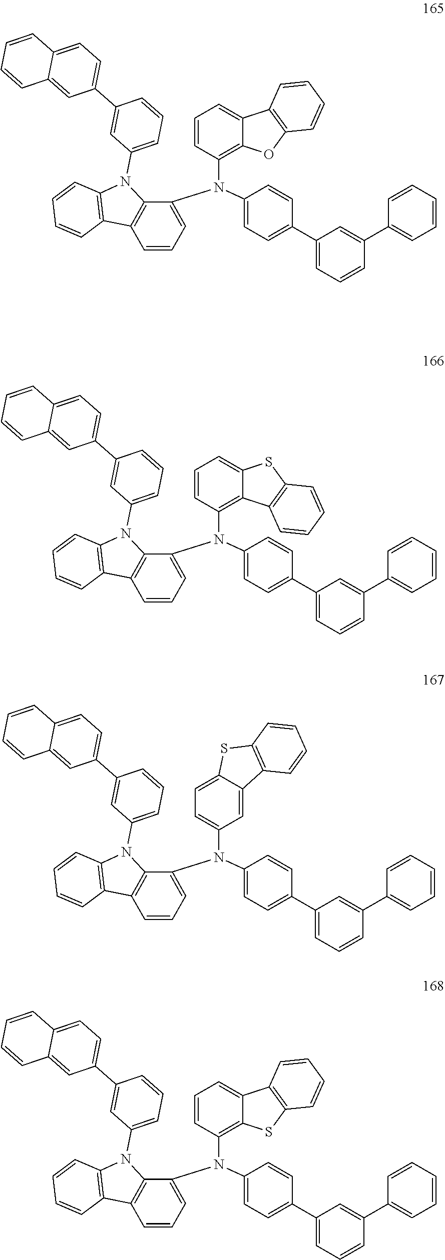 [SMILES: c1ccc(-c2cccc(-c3ccc(N(c4cccc5c4oc4ccccc45)c4cccc5c6ccccc6n(-c6cccc(-c7ccc8ccccc8c7)c6)c45)cc3)c2)cc1 ; c1ccc(-c2cccc(-c3ccc(N(c4cccc5sc6ccccc6c45)c4cccc5c6ccccc6n(-c6cccc(-c7ccc8ccccc8c7)c6)c45)cc3)c2)cc1 ; c1ccc(-c2cccc(-c3ccc(N(c4ccc5sc6ccccc6c5c4)c4cccc5c6ccccc6n(-c6cccc(-c7ccc8ccccc8c7)c6)c45)cc3)c2)cc1 ; c1ccc(-c2cccc(-c3ccc(N(c4cccc5c4sc4ccccc45)c4cccc5c6ccccc6n(-c6cccc(-c7ccc8ccccc8c7)c6)c45)cc3)c2)cc1]

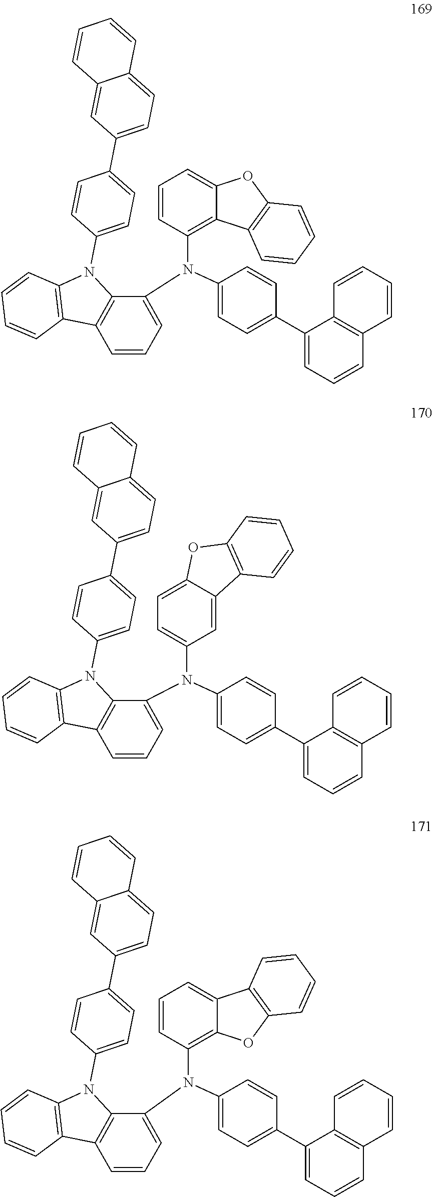 [SMILES: c1ccc2cc(-c3ccc(-n4c5ccccc5c5cccc(N(c6ccc(-c7cccc8ccccc78)cc6)c6cccc7oc8ccccc8c67)c54)cc3)ccc2c1 ; c1ccc2cc(-c3ccc(-n4c5ccccc5c5cccc(N(c6ccc(-c7cccc8ccccc78)cc6)c6ccc7oc8ccccc8c7c6)c54)cc3)ccc2c1 ; c1ccc2cc(-c3ccc(-n4c5ccccc5c5cccc(N(c6ccc(-c7cccc8ccccc78)cc6)c6cccc7c6oc6ccccc67)c54)cc3)ccc2c1]

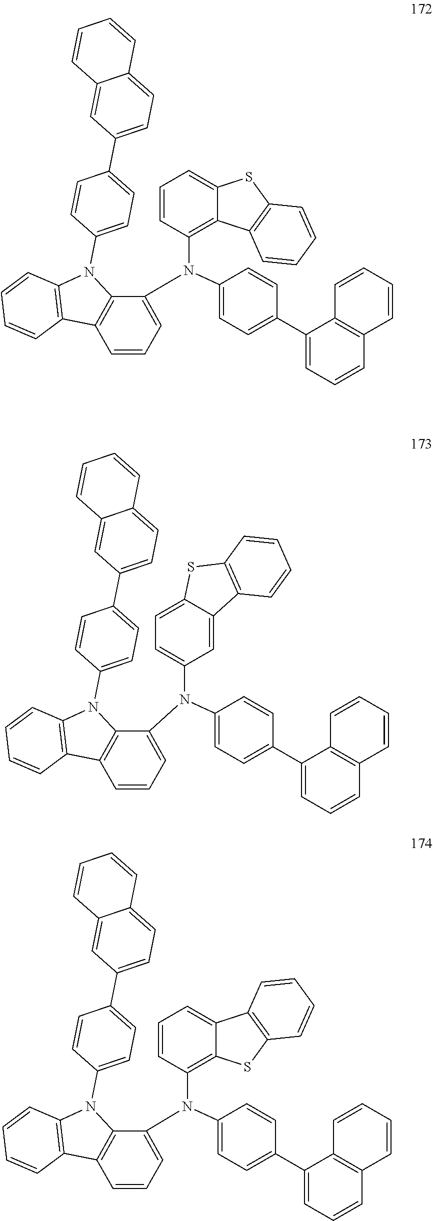 [SMILES: c1ccc2cc(-c3ccc(-n4c5ccccc5c5cccc(N(c6ccc(-c7cccc8ccccc78)cc6)c6cccc7sc8ccccc8c67)c54)cc3)ccc2c1 ; c1ccc2cc(-c3ccc(-n4c5ccccc5c5cccc(N(c6ccc(-c7cccc8ccccc78)cc6)c6ccc7sc8ccccc8c7c6)c54)cc3)ccc2c1 ; c1ccc2cc(-c3ccc(-n4c5ccccc5c5cccc(N(c6ccc(-c7cccc8ccccc78)cc6)c6cccc7c6sc6ccccc67)c54)cc3)ccc2c1]

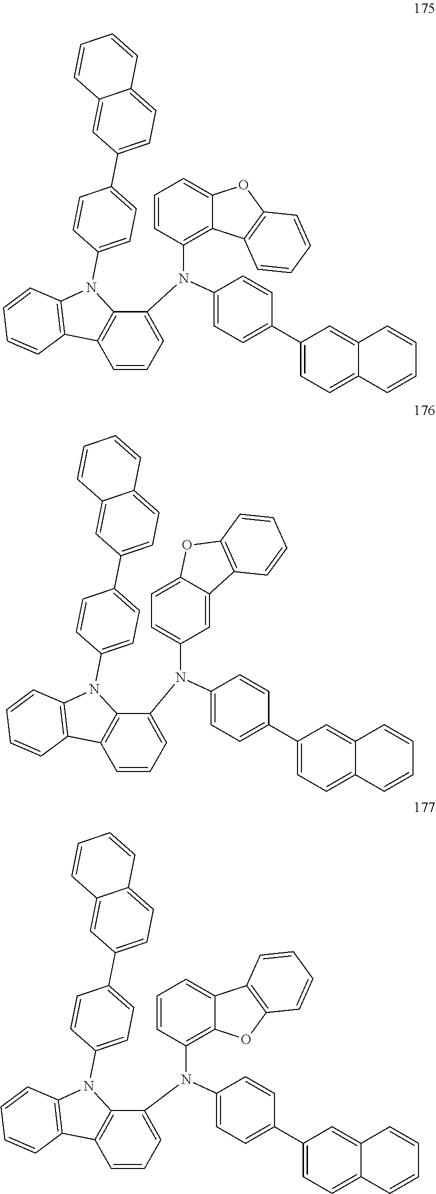 [SMILES: c1ccc2cc(-c3ccc(N(c4cccc5oc6ccccc6c45)c4cccc5c6ccccc6n(-c6ccc(-c7ccc8ccccc8c7)cc6)c45)cc3)ccc2c1 ; c1ccc2cc(-c3ccc(N(c4ccc5oc6ccccc6c5c4)c4cccc5c6ccccc6n(-c6ccc(-c7ccc8ccccc8c7)cc6)c45)cc3)ccc2c1 ; c1ccc2cc(-c3ccc(N(c4cccc5c4oc4ccccc45)c4cccc5c6ccccc6n(-c6ccc(-c7ccc8ccccc8c7)cc6)c45)cc3)ccc2c1]

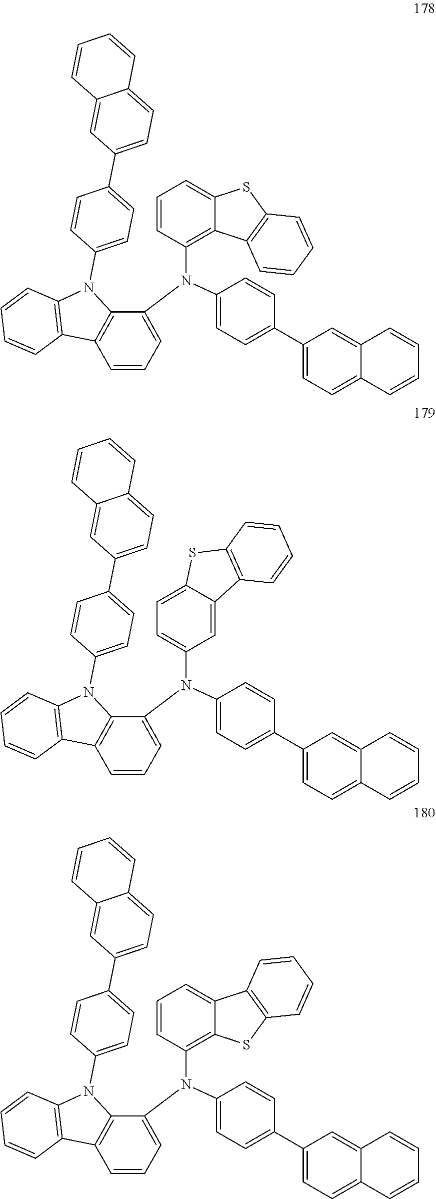 [SMILES: c1ccc2cc(-c3ccc(N(c4cccc5sc6ccccc6c45)c4cccc5c6ccccc6n(-c6ccc(-c7ccc8ccccc8c7)cc6)c45)cc3)ccc2c1 ; c1ccc2cc(-c3ccc(N(c4ccc5sc6ccccc6c5c4)c4cccc5c6ccccc6n(-c6ccc(-c7ccc8ccccc8c7)cc6)c45)cc3)ccc2c1 ; c1ccc2cc(-c3ccc(N(c4cccc5c4sc4ccccc45)c4cccc5c6ccccc6n(-c6ccc(-c7ccc8ccccc8c7)cc6)c45)cc3)ccc2c1]

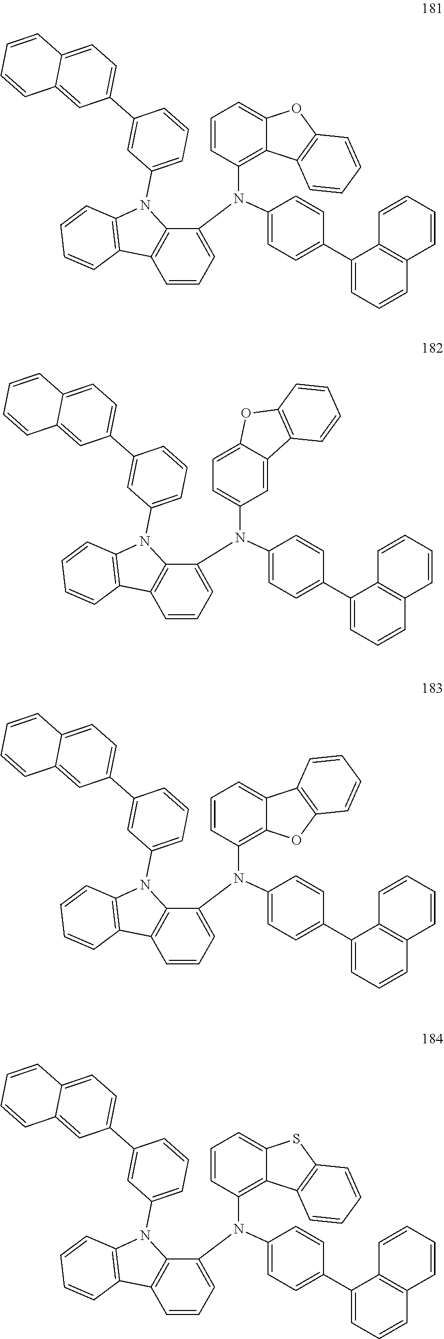 [SMILES: c1cc(-c2ccc3ccccc3c2)cc(-n2c3ccccc3c3cccc(N(c4ccc(-c5cccc6ccccc56)cc4)c4cccc5oc6ccccc6c45)c32)c1 ; c1cc(-c2ccc3ccccc3c2)cc(-n2c3ccccc3c3cccc(N(c4ccc(-c5cccc6ccccc56)cc4)c4ccc5oc6ccccc6c5c4)c32)c1 ; c1cc(-c2ccc3ccccc3c2)cc(-n2c3ccccc3c3cccc(N(c4ccc(-c5cccc6ccccc56)cc4)c4cccc5c4oc4ccccc45)c32)c1 ; c1cc(-c2ccc3ccccc3c2)cc(-n2c3ccccc3c3cccc(N(c4ccc(-c5cccc6ccccc56)cc4)c4cccc5sc6ccccc6c45)c32)c1]

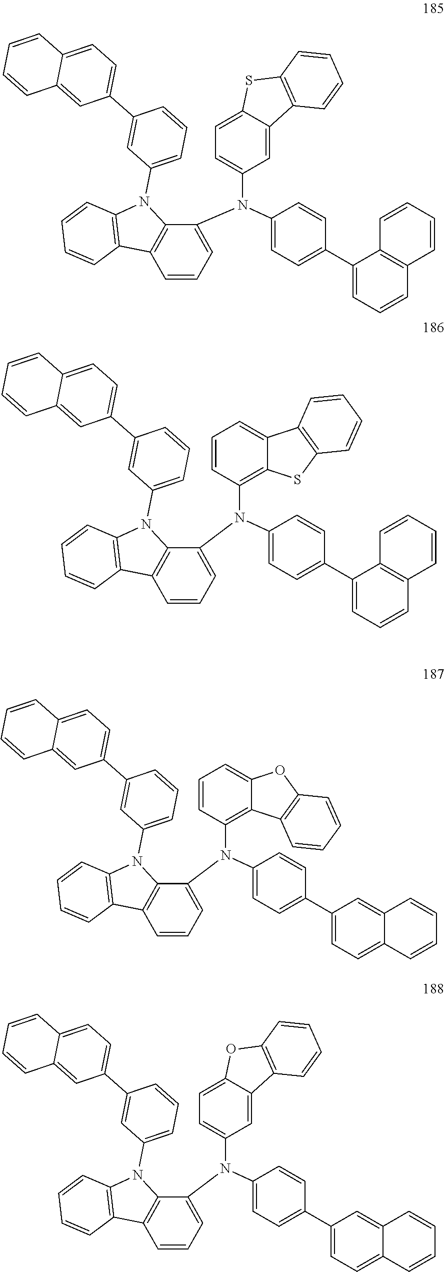 [SMILES: c1cc(-c2ccc3ccccc3c2)cc(-n2c3ccccc3c3cccc(N(c4ccc(-c5cccc6ccccc56)cc4)c4ccc5sc6ccccc6c5c4)c32)c1 ; c1cc(-c2ccc3ccccc3c2)cc(-n2c3ccccc3c3cccc(N(c4ccc(-c5cccc6ccccc56)cc4)c4cccc5c4sc4ccccc45)c32)c1 ; c1cc(-c2ccc3ccccc3c2)cc(-n2c3ccccc3c3cccc(N(c4ccc(-c5ccc6ccccc6c5)cc4)c4cccc5oc6ccccc6c45)c32)c1 ; c1cc(-c2ccc3ccccc3c2)cc(-n2c3ccccc3c3cccc(N(c4ccc(-c5ccc6ccccc6c5)cc4)c4ccc5oc6ccccc6c5c4)c32)c1]

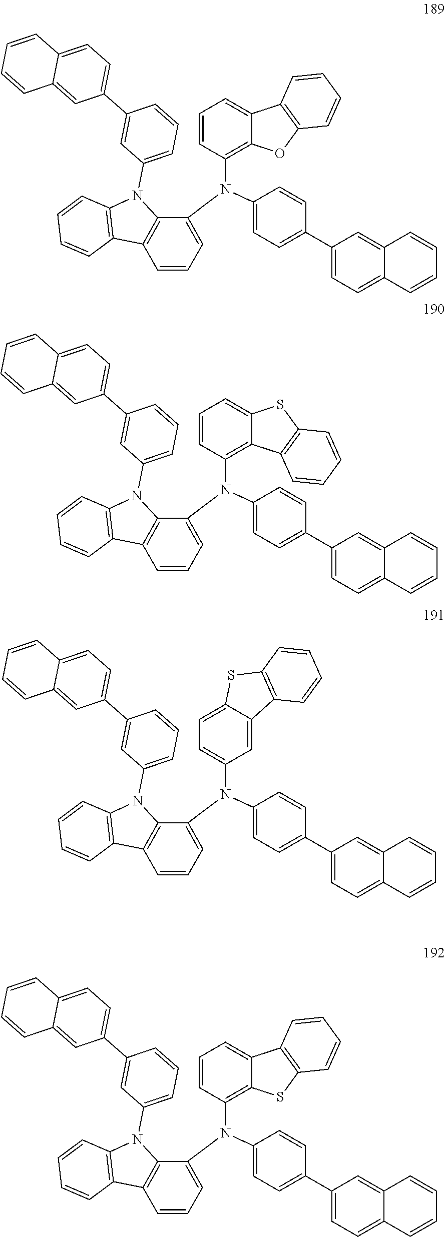 [SMILES: c1cc(-c2ccc3ccccc3c2)cc(-n2c3ccccc3c3cccc(N(c4ccc(-c5ccc6ccccc6c5)cc4)c4cccc5c4oc4ccccc45)c32)c1 ; c1cc(-c2ccc3ccccc3c2)cc(-n2c3ccccc3c3cccc(N(c4ccc(-c5ccc6ccccc6c5)cc4)c4cccc5sc6ccccc6c45)c32)c1 ; c1cc(-c2ccc3ccccc3c2)cc(-n2c3ccccc3c3cccc(N(c4ccc(-c5ccc6ccccc6c5)cc4)c4ccc5sc6ccccc6c5c4)c32)c1 ; c1cc(-c2ccc3ccccc3c2)cc(-n2c3ccccc3c3cccc(N(c4ccc(-c5ccc6ccccc6c5)cc4)c4cccc5c4sc4ccccc45)c32)c1]

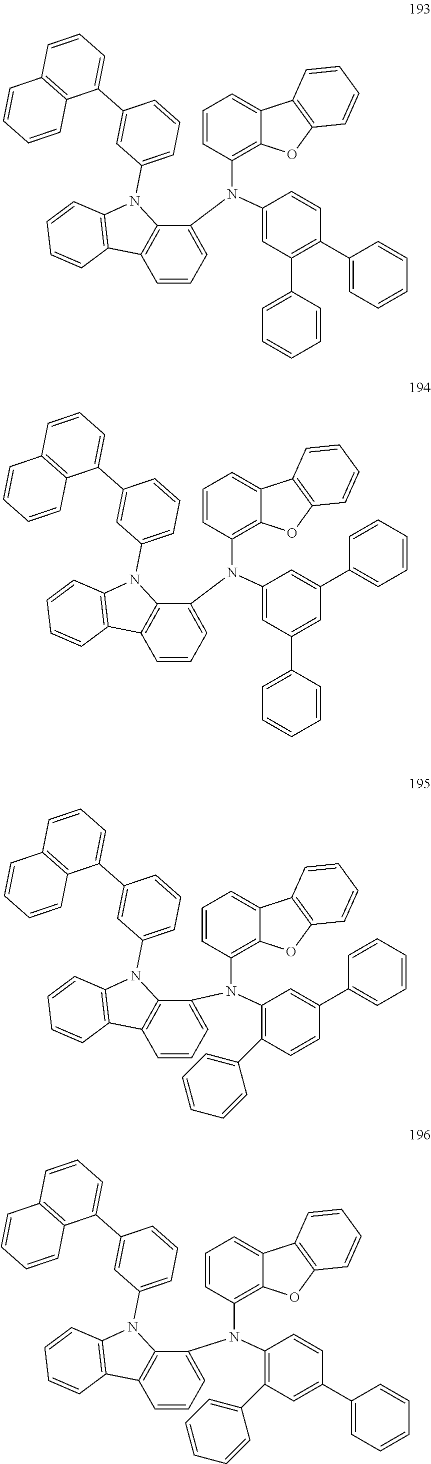 [SMILES: c1ccc(-c2ccc(N(c3cccc4c3oc3ccccc34)c3cccc4c5ccccc5n(-c5cccc(-c6cccc7ccccc67)c5)c34)cc2-c2ccccc2)cc1 ; c1ccc(-c2cc(-c3ccccc3)cc(N(c3cccc4c3oc3ccccc34)c3cccc4c5ccccc5n(-c5cccc(-c6cccc7ccccc67)c5)c34)c2)cc1 ; c1ccc(-c2ccc(-c3ccccc3)c(N(c3cccc4c3oc3ccccc34)c3cccc4c5ccccc5n(-c5cccc(-c6cccc7ccccc67)c5)c34)c2)cc1 ; c1ccc(-c2ccc(N(c3cccc4c3oc3ccccc34)c3cccc4c5ccccc5n(-c5cccc(-c6cccc7ccccc67)c5)c34)c(-c3ccccc3)c2)cc1]

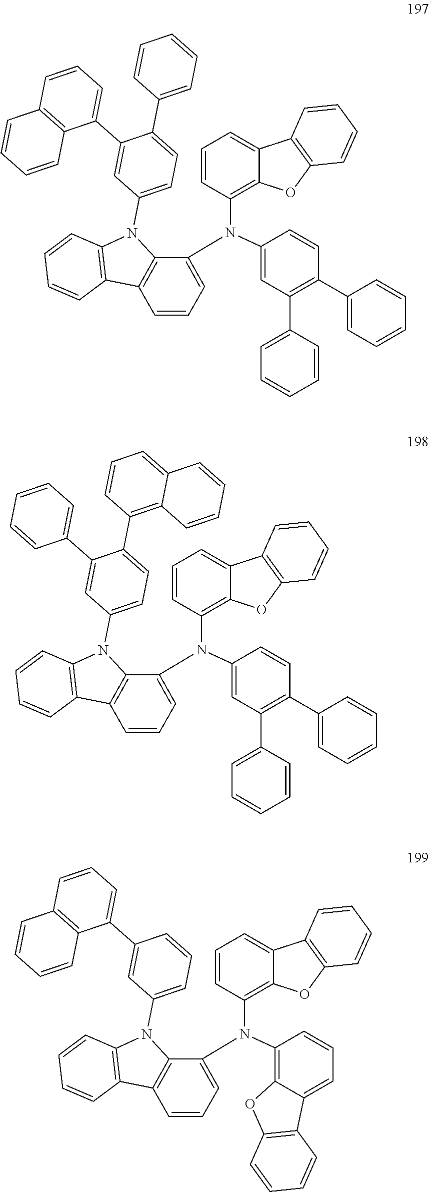 [SMILES: c1ccc(-c2ccc(N(c3cccc4c3oc3ccccc34)c3cccc4c5ccccc5n(-c5ccc(-c6ccccc6)c(-c6cccc7ccccc67)c5)c34)cc2-c2ccccc2)cc1 ; c1ccc(-c2ccc(N(c3cccc4c3oc3ccccc34)c3cccc4c5ccccc5n(-c5ccc(-c6cccc7ccccc67)c(-c6ccccc6)c5)c34)cc2-c2ccccc2)cc1 ; c1cc(-c2cccc3ccccc23)cc(-n2c3ccccc3c3cccc(N(c4cccc5c4oc4ccccc45)c4cccc5c4oc4ccccc45)c32)c1]

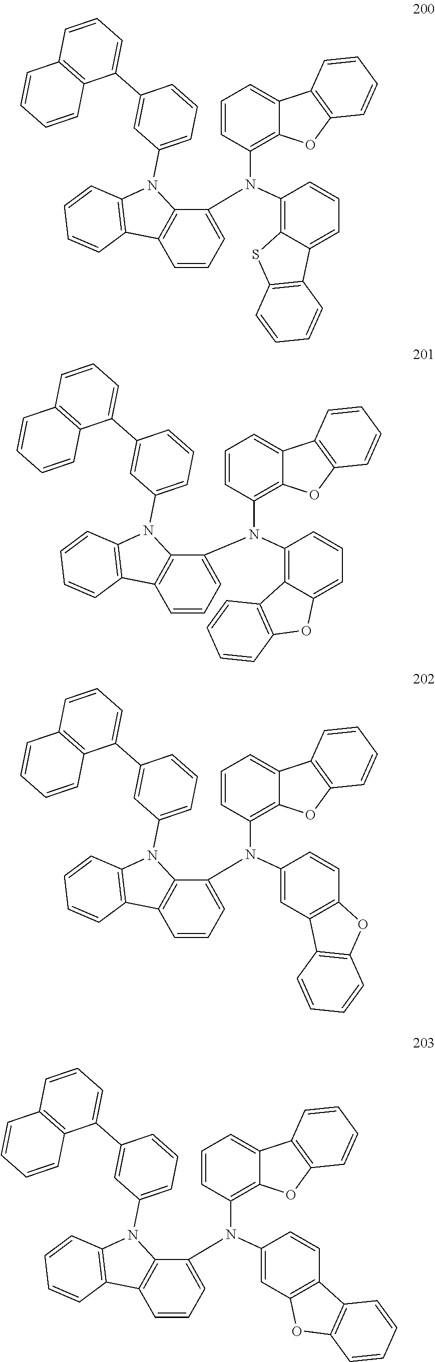 [SMILES: c1cc(-c2cccc3ccccc23)cc(-n2c3ccccc3c3cccc(N(c4cccc5c4oc4ccccc45)c4cccc5c4sc4ccccc45)c32)c1 ; c1cc(-c2cccc3ccccc23)cc(-n2c3ccccc3c3cccc(N(c4cccc5c4oc4ccccc45)c4cccc5oc6ccccc6c45)c32)c1 ; c1cc(-c2cccc3ccccc23)cc(-n2c3ccccc3c3cccc(N(c4ccc5oc6ccccc6c5c4)c4cccc5c4oc4ccccc45)c32)c1 ; c1cc(-c2cccc3ccccc23)cc(-n2c3ccccc3c3cccc(N(c4ccc5c(c4)oc4ccccc45)c4cccc5c4oc4ccccc45)c32)c1]

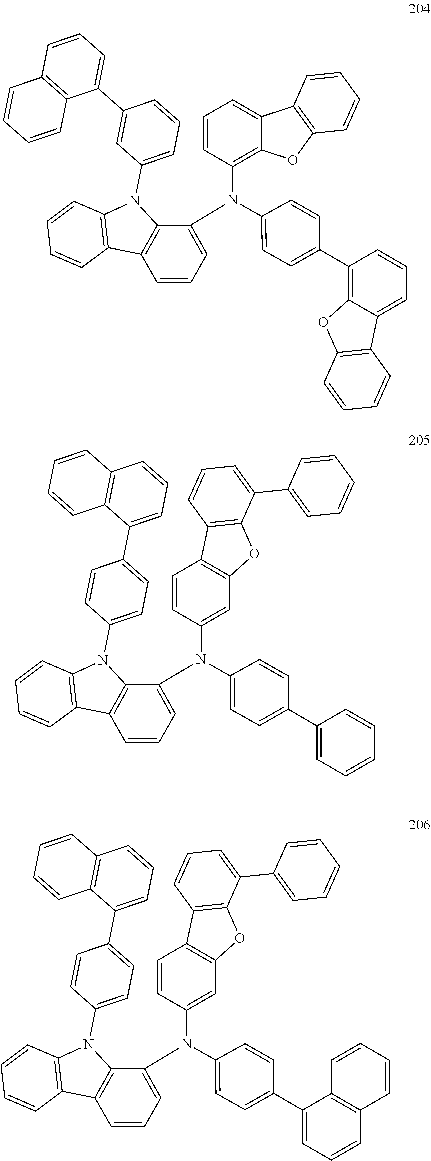

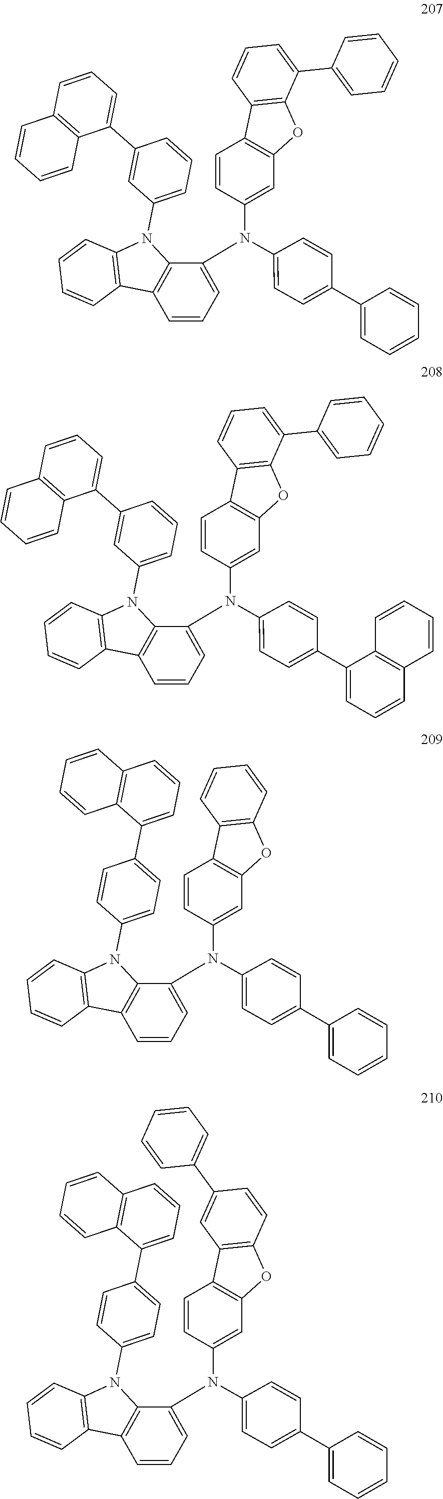 [SMILES: c1ccc(-c2ccc(N(c3ccc4c(c3)oc3c(-c5ccccc5)cccc34)c3cccc4c5ccccc5n(-c5cccc(-c6cccc7ccccc67)c5)c34)cc2)cc1 ; c1ccc(-c2cccc3c2oc2cc(N(c4ccc(-c5cccc6ccccc56)cc4)c4cccc5c6ccccc6n(-c6cccc(-c7cccc8ccccc78)c6)c45)ccc23)cc1 ; c1ccc(-c2ccc(N(c3ccc4c(c3)oc3ccccc34)c3cccc4c5ccccc5n(-c5ccc(-c6cccc7ccccc67)cc5)c34)cc2)cc1 ; c1ccc(-c2ccc(N(c3ccc4c(c3)oc3ccc(-c5ccccc5)cc34)c3cccc4c5ccccc5n(-c5ccc(-c6cccc7ccccc67)cc5)c34)cc2)cc1]

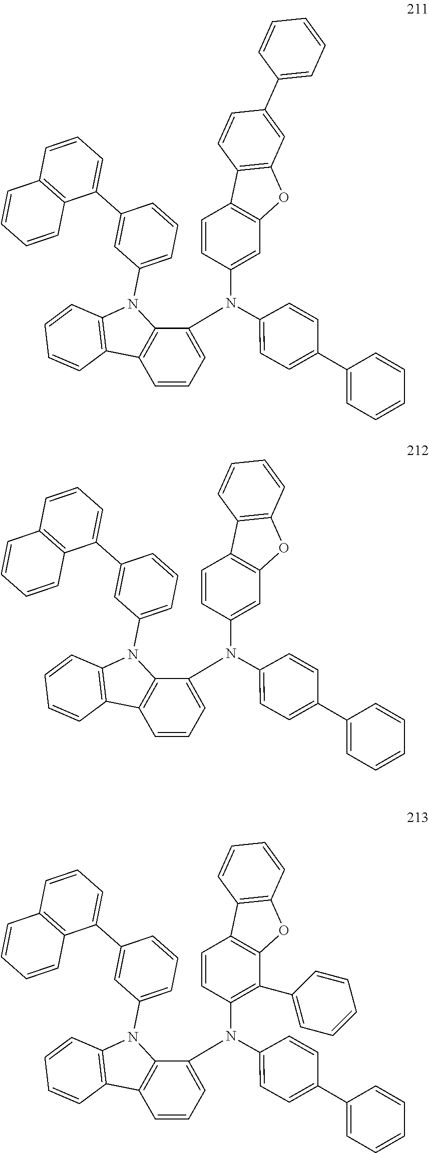 [SMILES: c1ccc(-c2ccc(N(c3ccc4c(c3)oc3cc(-c5ccccc5)ccc34)c3cccc4c5ccccc5n(-c5cccc(-c6cccc7ccccc67)c5)c34)cc2)cc1 ; c1ccc(-c2ccc(N(c3ccc4c(c3)oc3ccccc34)c3cccc4c5ccccc5n(-c5cccc(-c6cccc7ccccc67)c5)c34)cc2)cc1 ; c1ccc(-c2ccc(N(c3ccc4c(oc5ccccc54)c3-c3ccccc3)c3cccc4c5ccccc5n(-c5cccc(-c6cccc7ccccc67)c5)c34)cc2)cc1]

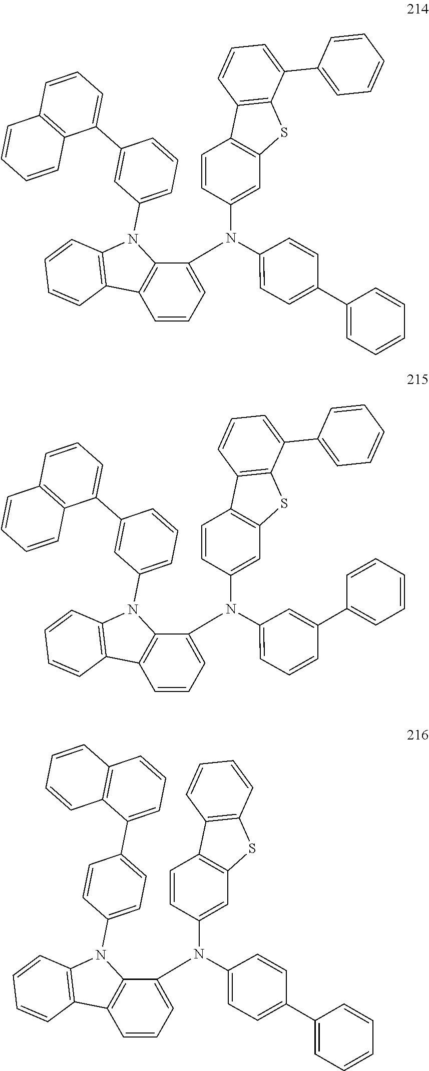 [SMILES: c1ccc(-c2ccc(N(c3ccc4c(c3)sc3c(-c5ccccc5)cccc34)c3cccc4c5ccccc5n(-c5cccc(-c6cccc7ccccc67)c5)c34)cc2)cc1 ; c1ccc(-c2cccc(N(c3ccc4c(c3)sc3c(-c5ccccc5)cccc34)c3cccc4c5ccccc5n(-c5cccc(-c6cccc7ccccc67)c5)c34)c2)cc1 ; c1ccc(-c2ccc(N(c3ccc4c(c3)sc3ccccc34)c3cccc4c5ccccc5n(-c5ccc(-c6cccc7ccccc67)cc5)c34)cc2)cc1]

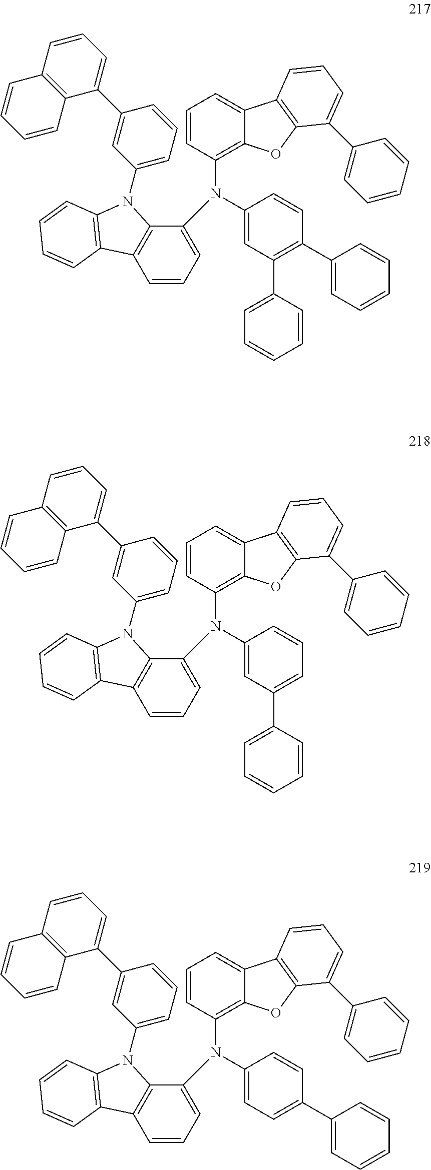 [SMILES: c1ccc(-c2ccc(N(c3cccc4c3oc3c(-c5ccccc5)cccc34)c3cccc4c5ccccc5n(-c5cccc(-c6cccc7ccccc67)c5)c34)cc2-c2ccccc2)cc1 ; c1ccc(-c2cccc(N(c3cccc4c3oc3c(-c5ccccc5)cccc34)c3cccc4c5ccccc5n(-c5cccc(-c6cccc7ccccc67)c5)c34)c2)cc1 ; c1ccc(-c2ccc(N(c3cccc4c3oc3c(-c5ccccc5)cccc34)c3cccc4c5ccccc5n(-c5cccc(-c6cccc7ccccc67)c5)c34)cc2)cc1]

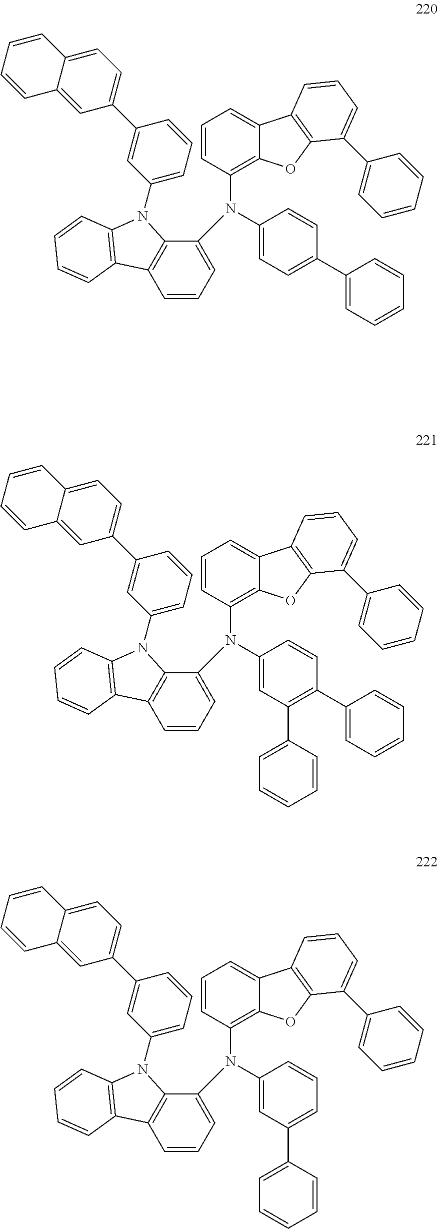 [SMILES: c1ccc(-c2ccc(N(c3cccc4c3oc3c(-c5ccccc5)cccc34)c3cccc4c5ccccc5n(-c5cccc(-c6ccc7ccccc7c6)c5)c34)cc2)cc1 ; c1ccc(-c2ccc(N(c3cccc4c3oc3c(-c5ccccc5)cccc34)c3cccc4c5ccccc5n(-c5cccc(-c6ccc7ccccc7c6)c5)c34)cc2-c2ccccc2)cc1 ; c1ccc(-c2cccc(N(c3cccc4c3oc3c(-c5ccccc5)cccc34)c3cccc4c5ccccc5n(-c5cccc(-c6ccc7ccccc7c6)c5)c34)c2)cc1]

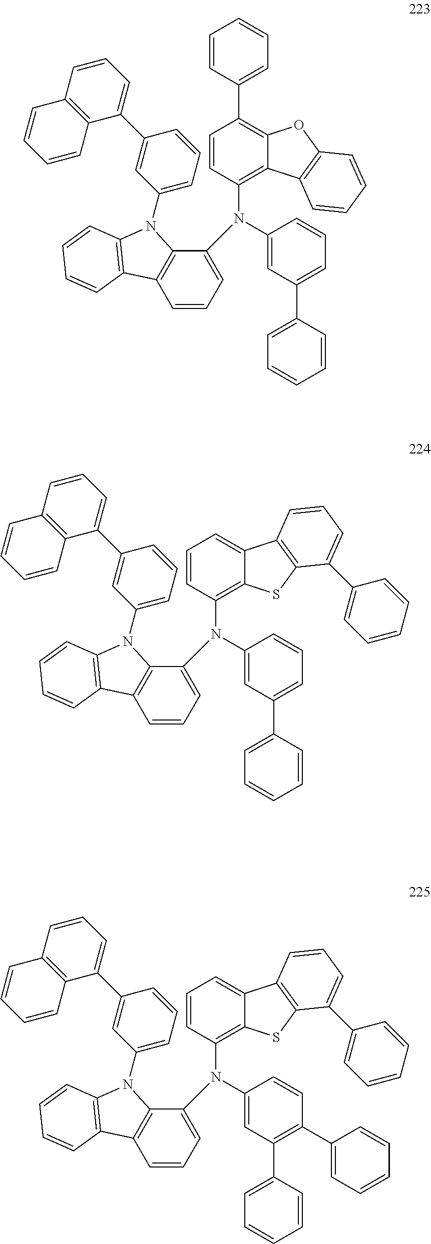 [SMILES: c1ccc(-c2cccc(N(c3ccc(-c4ccccc4)c4oc5ccccc5c34)c3cccc4c5ccccc5n(-c5cccc(-c6cccc7ccccc67)c5)c34)c2)cc1 ; c1ccc(-c2cccc(N(c3cccc4c3sc3c(-c5ccccc5)cccc34)c3cccc4c5ccccc5n(-c5cccc(-c6cccc7ccccc67)c5)c34)c2)cc1 ; c1ccc(-c2ccc(N(c3cccc4c3sc3c(-c5ccccc5)cccc34)c3cccc4c5ccccc5n(-c5cccc(-c6cccc7ccccc67)c5)c34)cc2-c2ccccc2)cc1]

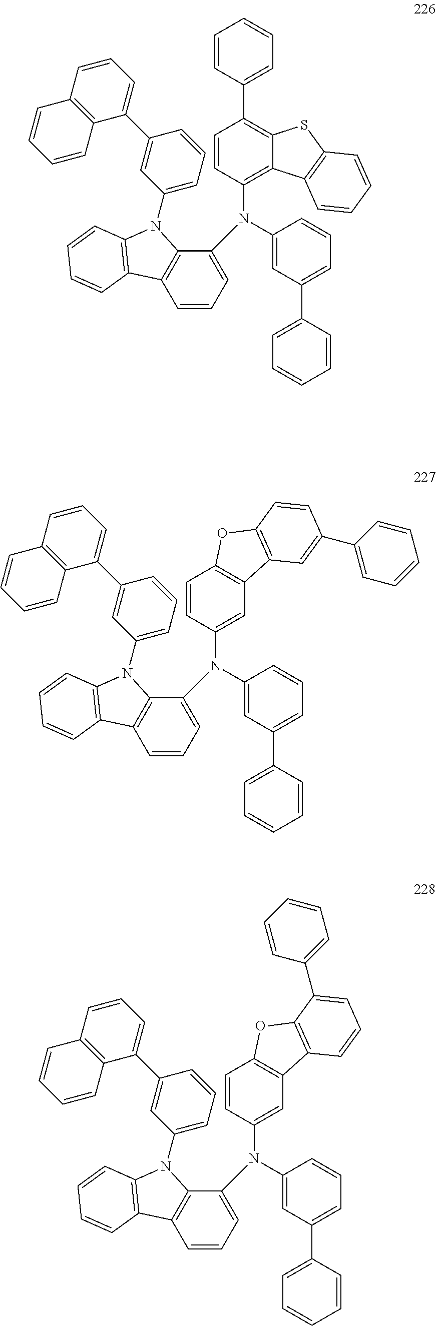 [SMILES: c1ccc(-c2cccc(N(c3ccc(-c4ccccc4)c4sc5ccccc5c34)c3cccc4c5ccccc5n(-c5cccc(-c6cccc7ccccc67)c5)c34)c2)cc1 ; c1ccc(-c2cccc(N(c3ccc4oc5ccc(-c6ccccc6)cc5c4c3)c3cccc4c5ccccc5n(-c5cccc(-c6cccc7ccccc67)c5)c34)c2)cc1 ; c1ccc(-c2cccc(N(c3ccc4oc5c(-c6ccccc6)cccc5c4c3)c3cccc4c5ccccc5n(-c5cccc(-c6cccc7ccccc67)c5)c34)c2)cc1]

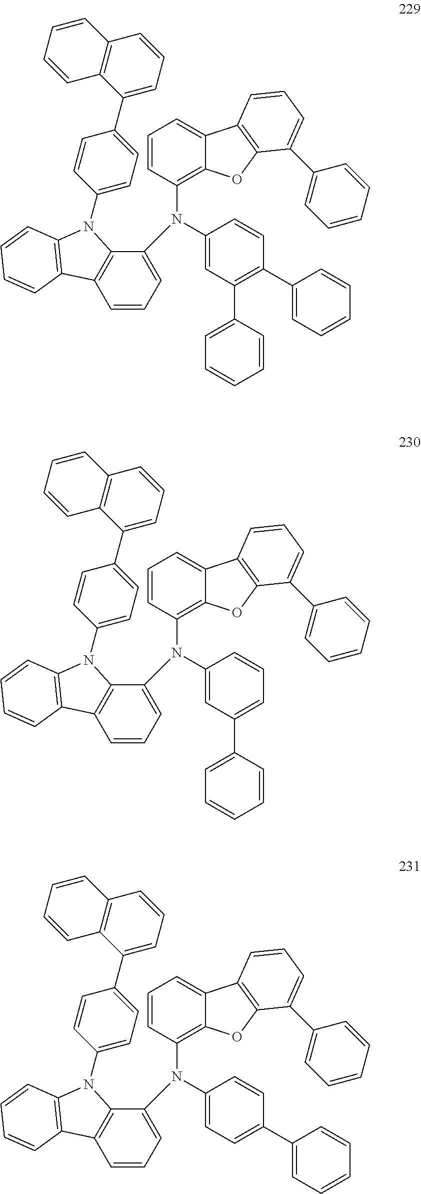 [SMILES: c1ccc(-c2ccc(N(c3cccc4c3oc3c(-c5ccccc5)cccc34)c3cccc4c5ccccc5n(-c5ccc(-c6cccc7ccccc67)cc5)c34)cc2-c2ccccc2)cc1 ; c1ccc(-c2cccc(N(c3cccc4c3oc3c(-c5ccccc5)cccc34)c3cccc4c5ccccc5n(-c5ccc(-c6cccc7ccccc67)cc5)c34)c2)cc1 ; c1ccc(-c2ccc(N(c3cccc4c3oc3c(-c5ccccc5)cccc34)c3cccc4c5ccccc5n(-c5ccc(-c6cccc7ccccc67)cc5)c34)cc2)cc1]

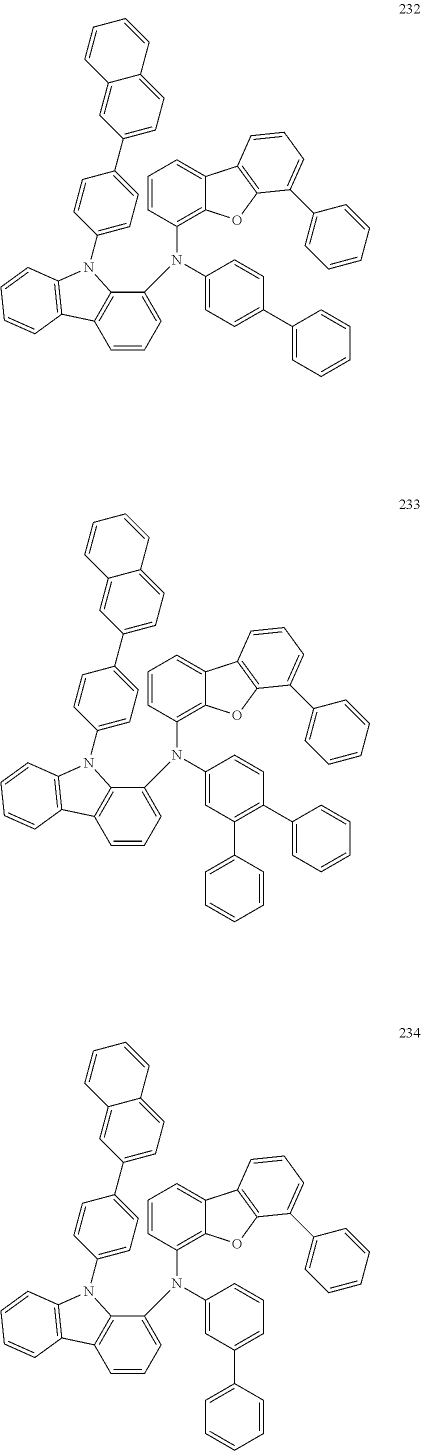 [SMILES: c1ccc(-c2ccc(N(c3cccc4c3oc3c(-c5ccccc5)cccc34)c3cccc4c5ccccc5n(-c5ccc(-c6ccc7ccccc7c6)cc5)c34)cc2)cc1 ; c1ccc(-c2ccc(N(c3cccc4c3oc3c(-c5ccccc5)cccc34)c3cccc4c5ccccc5n(-c5ccc(-c6ccc7ccccc7c6)cc5)c34)cc2-c2ccccc2)cc1 ; c1ccc(-c2cccc(N(c3cccc4c3oc3c(-c5ccccc5)cccc34)c3cccc4c5ccccc5n(-c5ccc(-c6ccc7ccccc7c6)cc5)c34)c2)cc1]

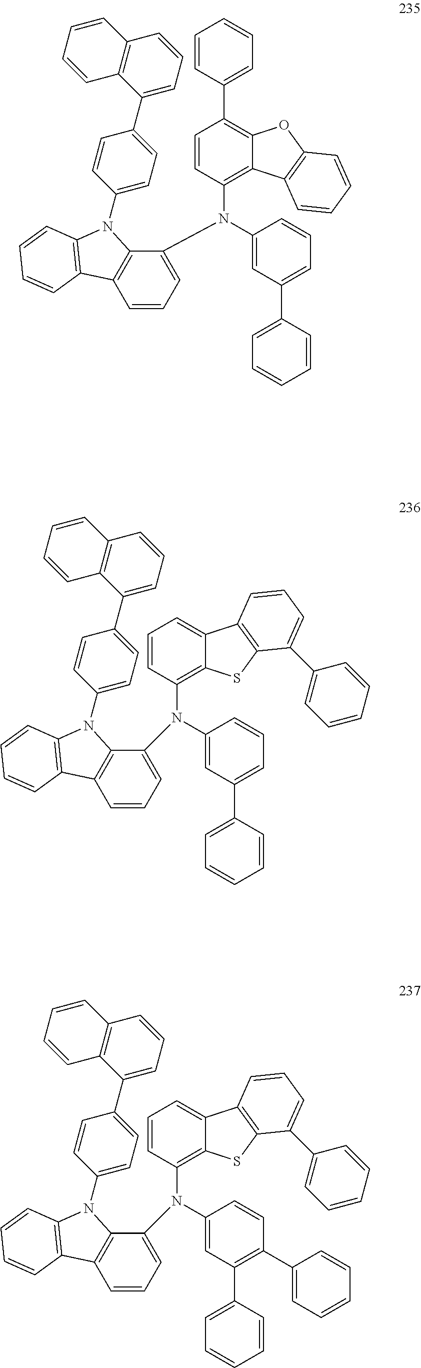 [SMILES: c1ccc(-c2cccc(N(c3ccc(-c4ccccc4)c4oc5ccccc5c34)c3cccc4c5ccccc5n(-c5ccc(-c6cccc7ccccc67)cc5)c34)c2)cc1 ; c1ccc(-c2cccc(N(c3cccc4c3sc3c(-c5ccccc5)cccc34)c3cccc4c5ccccc5n(-c5ccc(-c6cccc7ccccc67)cc5)c34)c2)cc1 ; c1ccc(-c2ccc(N(c3cccc4c3sc3c(-c5ccccc5)cccc34)c3cccc4c5ccccc5n(-c5ccc(-c6cccc7ccccc67)cc5)c34)cc2-c2ccccc2)cc1]

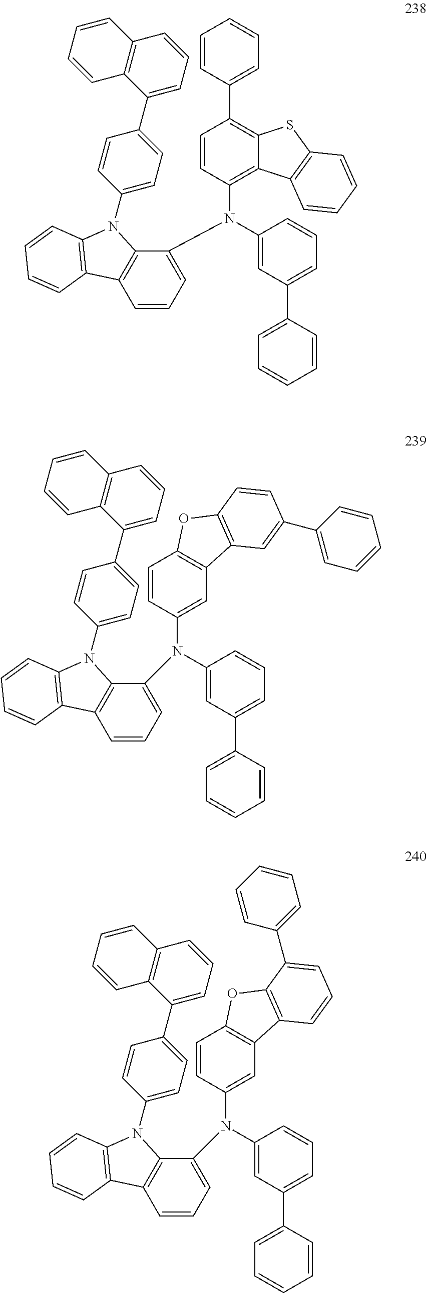 [SMILES: c1ccc(-c2cccc(N(c3ccc(-c4ccccc4)c4sc5ccccc5c34)c3cccc4c5ccccc5n(-c5ccc(-c6cccc7ccccc67)cc5)c34)c2)cc1 ; c1ccc(-c2cccc(N(c3ccc4oc5ccc(-c6ccccc6)cc5c4c3)c3cccc4c5ccccc5n(-c5ccc(-c6cccc7ccccc67)cc5)c34)c2)cc1 ; c1ccc(-c2cccc(N(c3ccc4oc5c(-c6ccccc6)cccc5c4c3)c3cccc4c5ccccc5n(-c5ccc(-c6cccc7ccccc67)cc5)c34)c2)cc1]

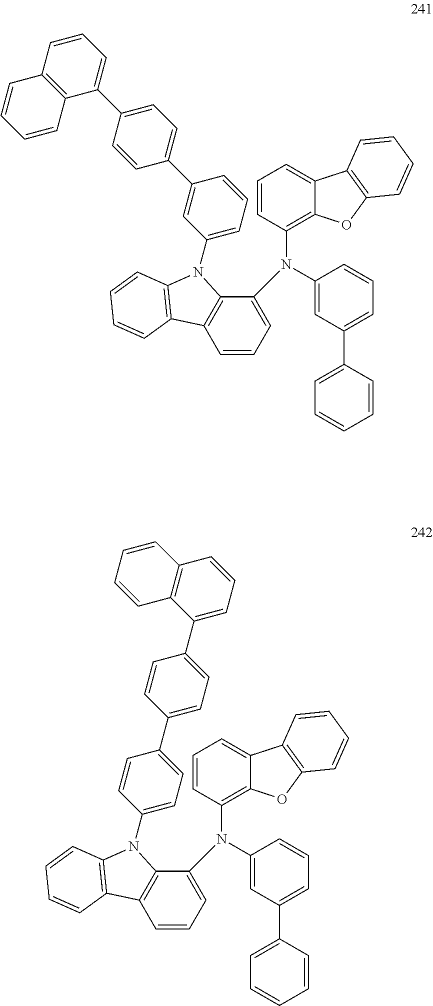 [SMILES: c1ccc(-c2cccc(N(c3cccc4c3oc3ccccc34)c3cccc4c5ccccc5n(-c5cccc(-c6ccc(-c7cccc8ccccc78)cc6)c5)c34)c2)cc1 ; c1ccc(-c2cccc(N(c3cccc4c3oc3ccccc34)c3cccc4c5ccccc5n(-c5ccc(-c6ccc(-c7cccc8ccccc78)cc6)cc5)c34)c2)cc1]

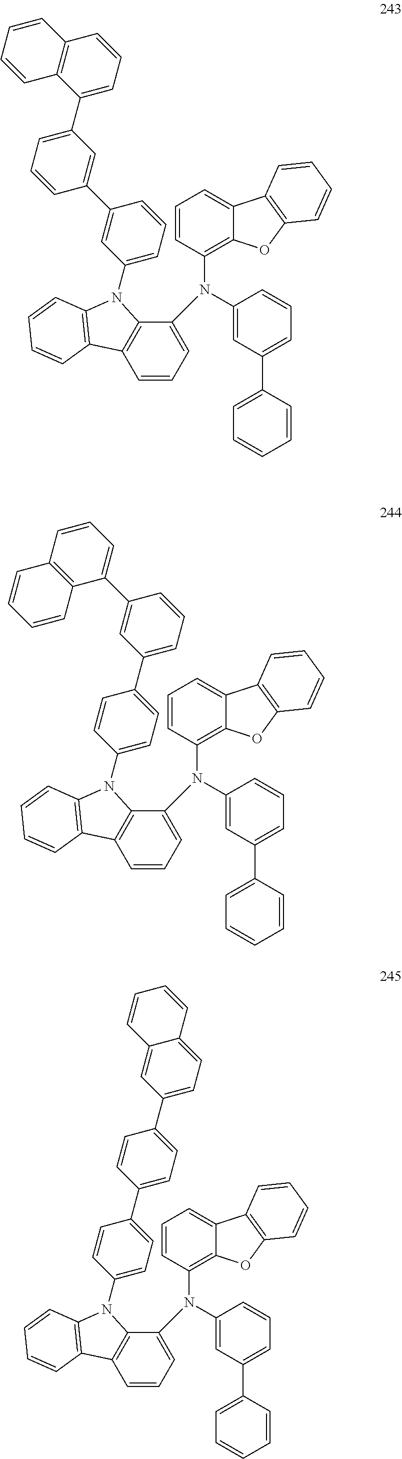 [SMILES: c1ccc(-c2cccc(N(c3cccc4c3oc3ccccc34)c3cccc4c5ccccc5n(-c5cccc(-c6cccc(-c7cccc8ccccc78)c6)c5)c34)c2)cc1 ; c1ccc(-c2cccc(N(c3cccc4c3oc3ccccc34)c3cccc4c5ccccc5n(-c5ccc(-c6cccc(-c7cccc8ccccc78)c6)cc5)c34)c2)cc1 ; c1ccc(-c2cccc(N(c3cccc4c3oc3ccccc34)c3cccc4c5ccccc5n(-c5ccc(-c6ccc(-c7ccc8ccccc8c7)cc6)cc5)c34)c2)cc1]

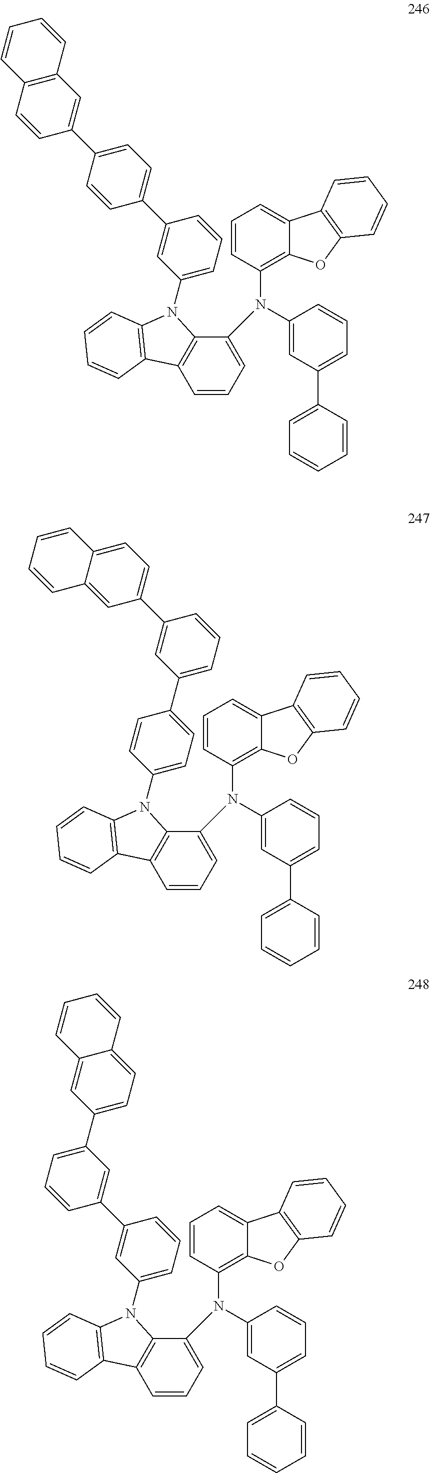 [SMILES: c1ccc(-c2cccc(N(c3cccc4c3oc3ccccc34)c3cccc4c5ccccc5n(-c5cccc(-c6ccc(-c7ccc8ccccc8c7)cc6)c5)c34)c2)cc1 ; c1ccc(-c2cccc(N(c3cccc4c3oc3ccccc34)c3cccc4c5ccccc5n(-c5ccc(-c6cccc(-c7ccc8ccccc8c7)c6)cc5)c34)c2)cc1 ; c1ccc(-c2cccc(N(c3cccc4c3oc3ccccc34)c3cccc4c5ccccc5n(-c5cccc(-c6cccc(-c7ccc8ccccc8c7)c6)c5)c34)c2)cc1]

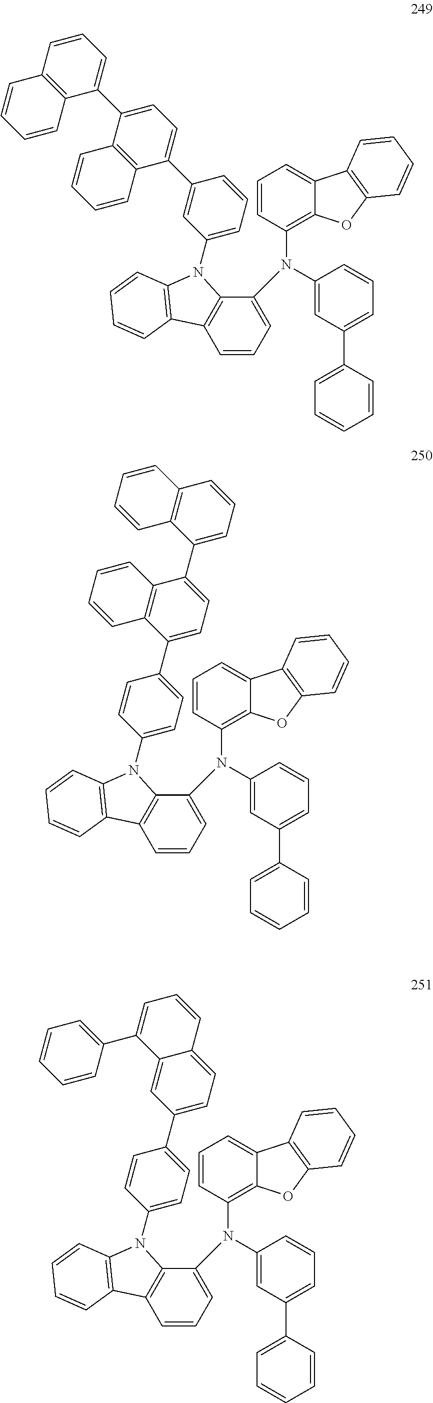 [SMILES: c1ccc(-c2cccc(N(c3cccc4c3oc3ccccc34)c3cccc4c5ccccc5n(-c5cccc(-c6ccc(-c7cccc8ccccc78)c7ccccc67)c5)c34)c2)cc1 ; c1ccc(-c2cccc(N(c3cccc4c3oc3ccccc34)c3cccc4c5ccccc5n(-c5ccc(-c6ccc(-c7cccc8ccccc78)c7ccccc67)cc5)c34)c2)cc1 ; c1ccc(-c2cccc(N(c3cccc4c3oc3ccccc34)c3cccc4c5ccccc5n(-c5ccc(-c6ccc7cccc(-c8ccccc8)c7c6)cc5)c34)c2)cc1]

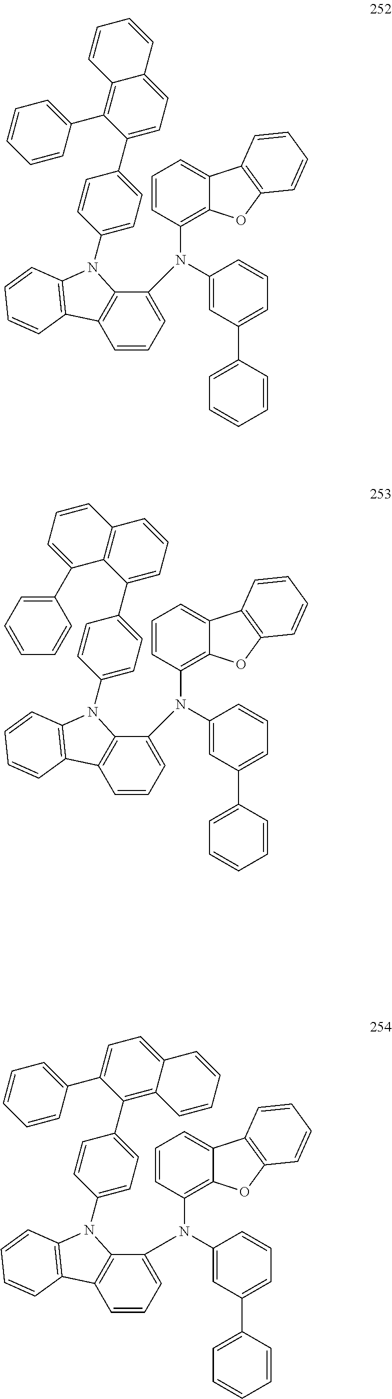 [SMILES: c1ccc(-c2cccc(N(c3cccc4c3oc3ccccc34)c3cccc4c5ccccc5n(-c5ccc(-c6ccc7ccccc7c6-c6ccccc6)cc5)c34)c2)cc1 ; c1ccc(-c2cccc(N(c3cccc4c3oc3ccccc34)c3cccc4c5ccccc5n(-c5ccc(-c6cccc7cccc(-c8ccccc8)c67)cc5)c34)c2)cc1 ; c1ccc(-c2cccc(N(c3cccc4c3oc3ccccc34)c3cccc4c5ccccc5n(-c5ccc(-c6c(-c7ccccc7)ccc7ccccc67)cc5)c34)c2)cc1]

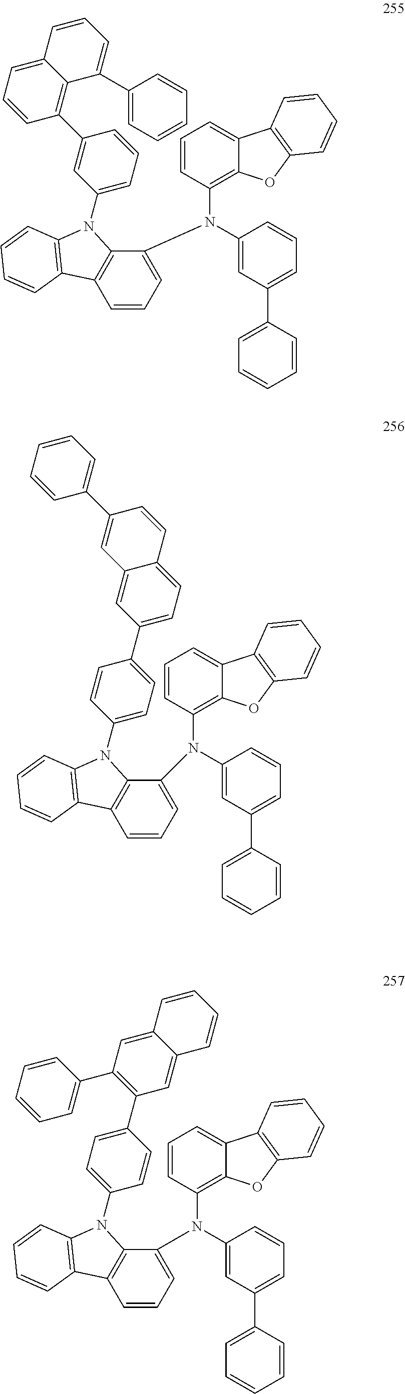 [SMILES: c1ccc(-c2cccc(N(c3cccc4c3oc3ccccc34)c3cccc4c5ccccc5n(-c5cccc(-c6cccc7cccc(-c8ccccc8)c67)c5)c34)c2)cc1 ; c1ccc(-c2cccc(N(c3cccc4c3oc3ccccc34)c3cccc4c5ccccc5n(-c5ccc(-c6ccc7ccc(-c8ccccc8)cc7c6)cc5)c34)c2)cc1 ; c1ccc(-c2cccc(N(c3cccc4c3oc3ccccc34)c3cccc4c5ccccc5n(-c5ccc(-c6cc7ccccc7cc6-c6ccccc6)cc5)c34)c2)cc1]

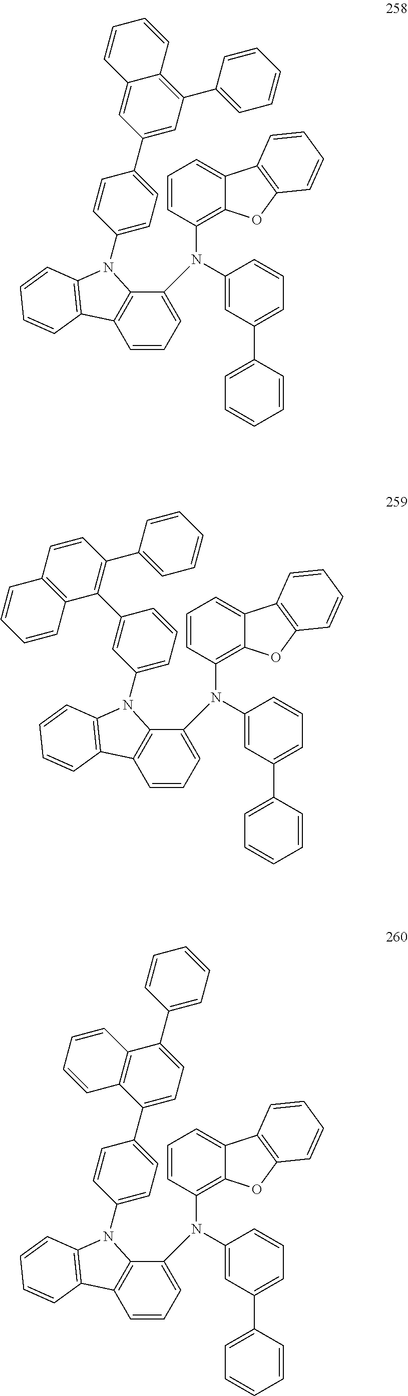 [SMILES: c1ccc(-c2cccc(N(c3cccc4c3oc3ccccc34)c3cccc4c5ccccc5n(-c5ccc(-c6cc(-c7ccccc7)c7ccccc7c6)cc5)c34)c2)cc1 ; c1ccc(-c2cccc(N(c3cccc4c3oc3ccccc34)c3cccc4c5ccccc5n(-c5cccc(-c6c(-c7ccccc7)ccc7ccccc67)c5)c34)c2)cc1 ; c1ccc(-c2cccc(N(c3cccc4c3oc3ccccc34)c3cccc4c5ccccc5n(-c5ccc(-c6ccc(-c7ccccc7)c7ccccc67)cc5)c34)c2)cc1]

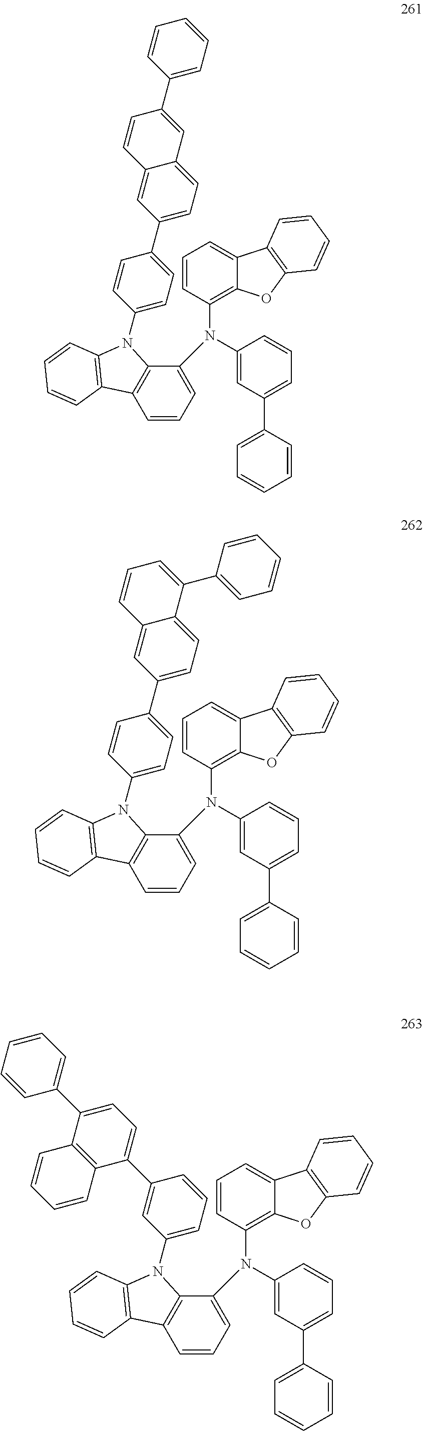

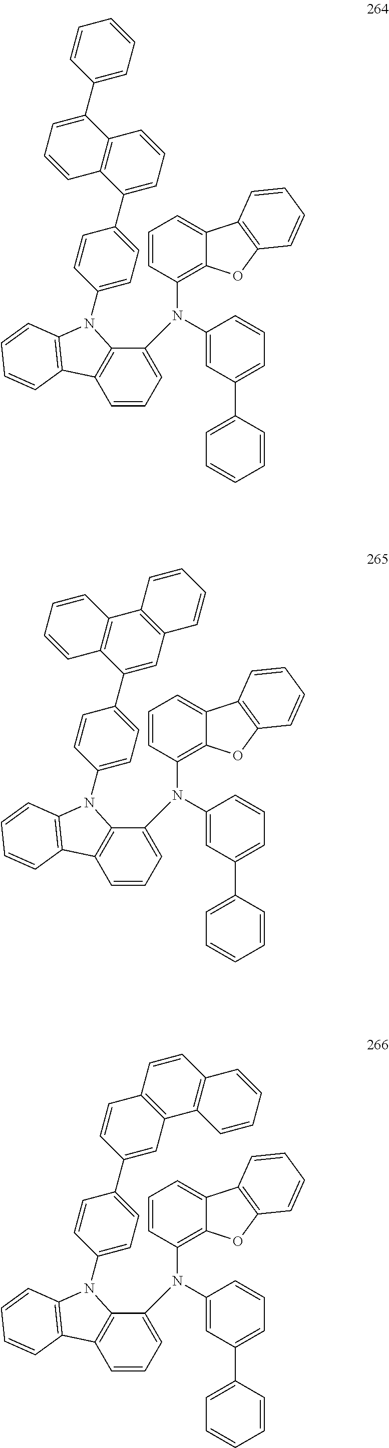 [SMILES: c1ccc(-c2cccc(N(c3cccc4c3oc3ccccc34)c3cccc4c5ccccc5n(-c5ccc(-c6cccc7c(-c8ccccc8)cccc67)cc5)c34)c2)cc1 ; c1ccc(-c2cccc(N(c3cccc4c3oc3ccccc34)c3cccc4c5ccccc5n(-c5ccc(-c6cc7ccccc7c7ccccc67)cc5)c34)c2)cc1 ; c1ccc(-c2cccc(N(c3cccc4c3oc3ccccc34)c3cccc4c5ccccc5n(-c5ccc(-c6ccc7ccc8ccccc8c7c6)cc5)c34)c2)cc1]

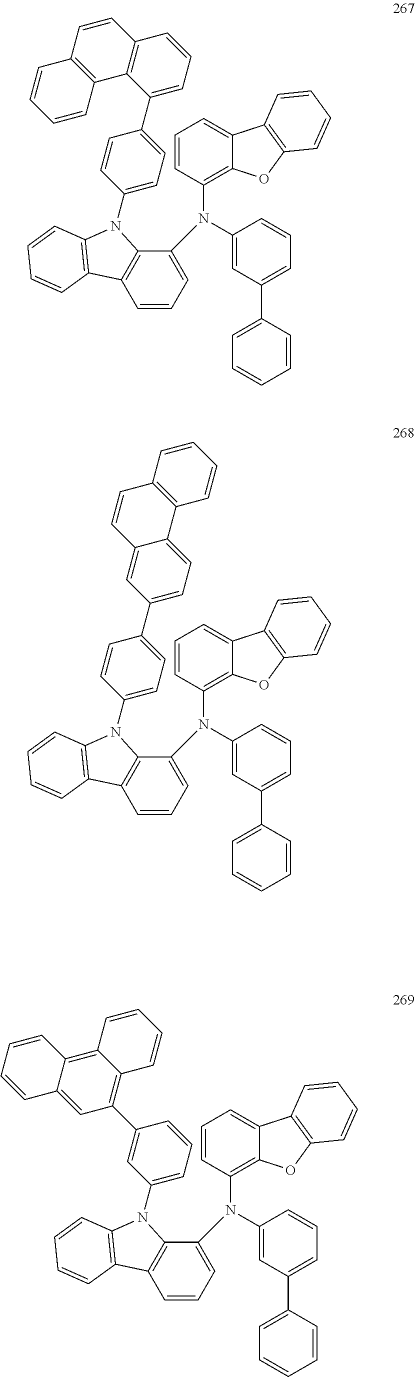 [SMILES: c1ccc(-c2cccc(N(c3cccc4c3oc3ccccc34)c3cccc4c5ccccc5n(-c5ccc(-c6cccc7ccc8ccccc8c67)cc5)c34)c2)cc1 ; c1ccc(-c2cccc(N(c3cccc4c3oc3ccccc34)c3cccc4c5ccccc5n(-c5ccc(-c6ccc7c(ccc8ccccc87)c6)cc5)c34)c2)cc1 ; c1ccc(-c2cccc(N(c3cccc4c3oc3ccccc34)c3cccc4c5ccccc5n(-c5cccc(-c6cc7ccccc7c7ccccc67)c5)c34)c2)cc1]

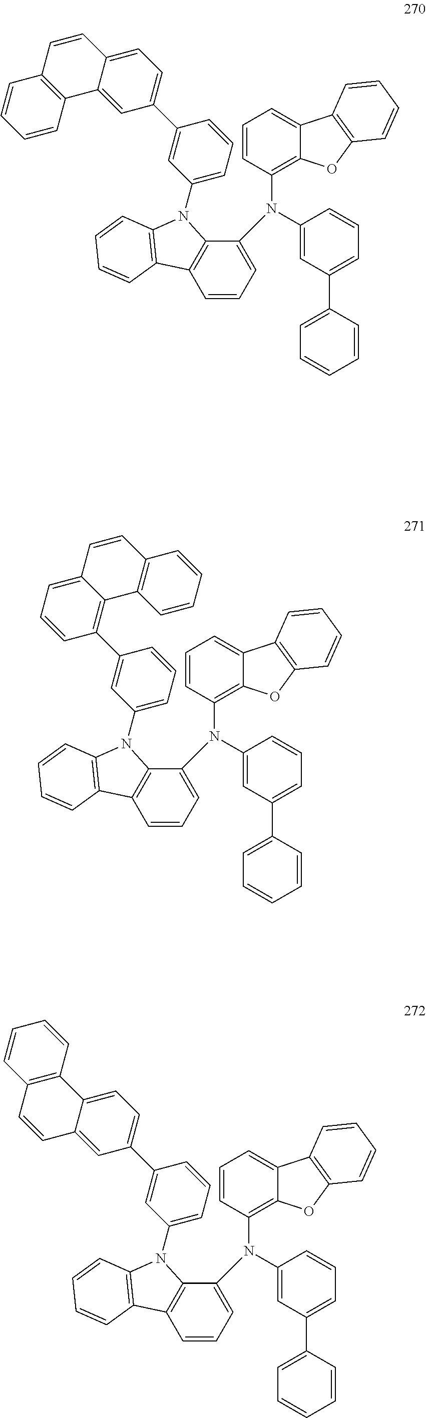 [SMILES: c1ccc(-c2cccc(N(c3cccc4c3oc3ccccc34)c3cccc4c5ccccc5n(-c5cccc(-c6ccc7ccc8ccccc8c7c6)c5)c34)c2)cc1 ; c1ccc(-c2cccc(N(c3cccc4c3oc3ccccc34)c3cccc4c5ccccc5n(-c5cccc(-c6cccc7ccc8ccccc8c67)c5)c34)c2)cc1 ; c1ccc(-c2cccc(N(c3cccc4c3oc3ccccc34)c3cccc4c5ccccc5n(-c5cccc(-c6ccc7c(ccc8ccccc87)c6)c5)c34)c2)cc1]

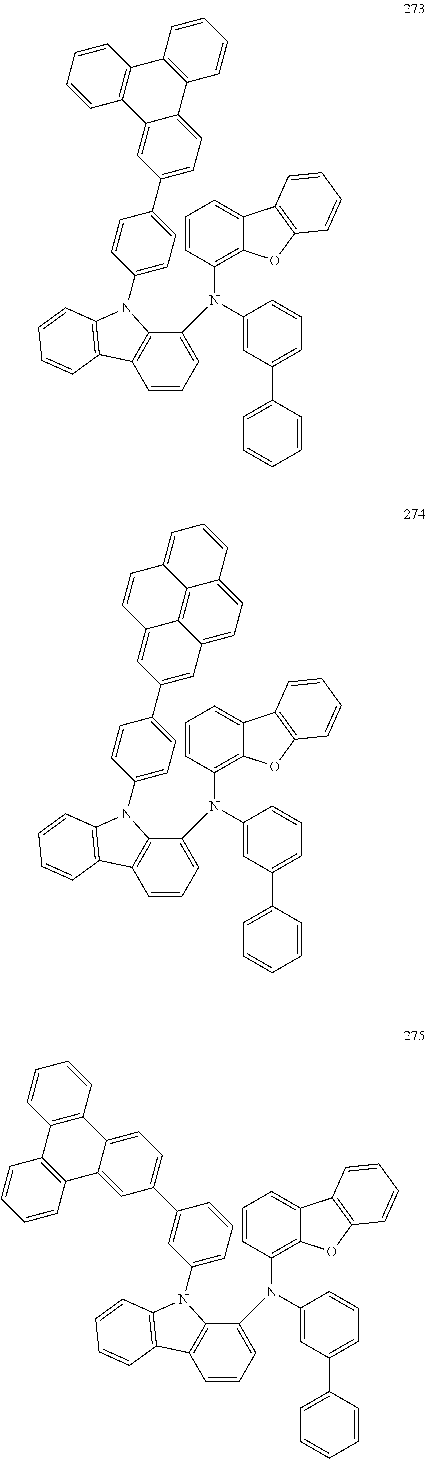 [SMILES: c1ccc(-c2cccc(N(c3cccc4c3oc3ccccc34)c3cccc4c5ccccc5n(-c5ccc(-c6ccc7c8ccccc8c8ccccc8c7c6)cc5)c34)c2)cc1 ; c1ccc(-c2cccc(N(c3cccc4c3oc3ccccc34)c3cccc4c5ccccc5n(-c5ccc(-c6cc7ccc8cccc9ccc(c6)c7c89)cc5)c34)c2)cc1 ; c1ccc(-c2cccc(N(c3cccc4c3oc3ccccc34)c3cccc4c5ccccc5n(-c5cccc(-c6ccc7c8ccccc8c8ccccc8c7c6)c5)c34)c2)cc1]

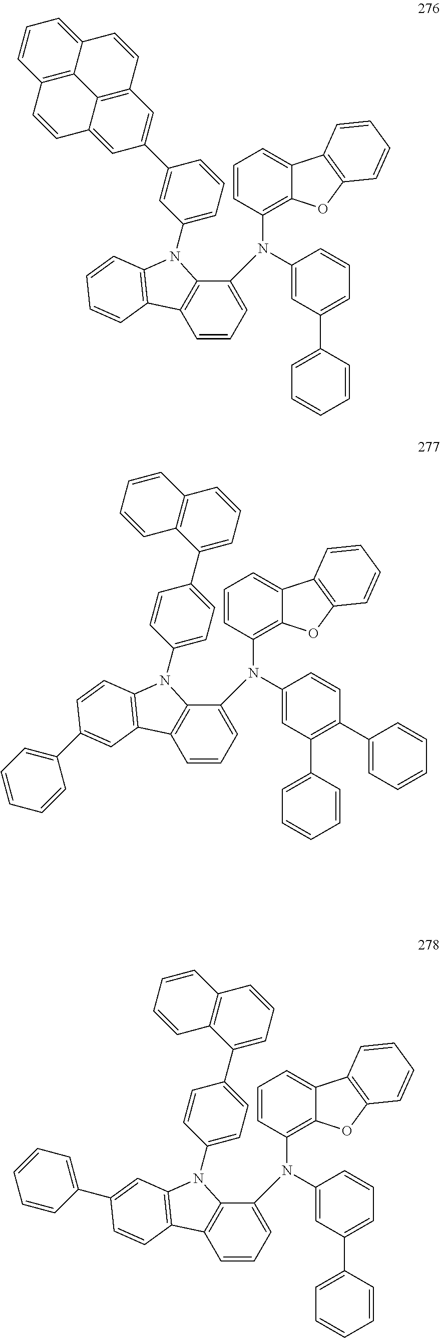 [SMILES: c1ccc(-c2cccc(N(c3cccc4c3oc3ccccc34)c3cccc4c5ccccc5n(-c5cccc(-c6cc7ccc8cccc9ccc(c6)c7c89)c5)c34)c2)cc1 ; c1ccc(-c2ccc3c(c2)c2cccc(N(c4ccc(-c5ccccc5)c(-c5ccccc5)c4)c4cccc5c4oc4ccccc45)c2n3-c2ccc(-c3cccc4ccccc34)cc2)cc1 ; c1ccc(-c2cccc(N(c3cccc4c3oc3ccccc34)c3cccc4c5ccc(-c6ccccc6)cc5n(-c5ccc(-c6cccc7ccccc67)cc5)c34)c2)cc1]

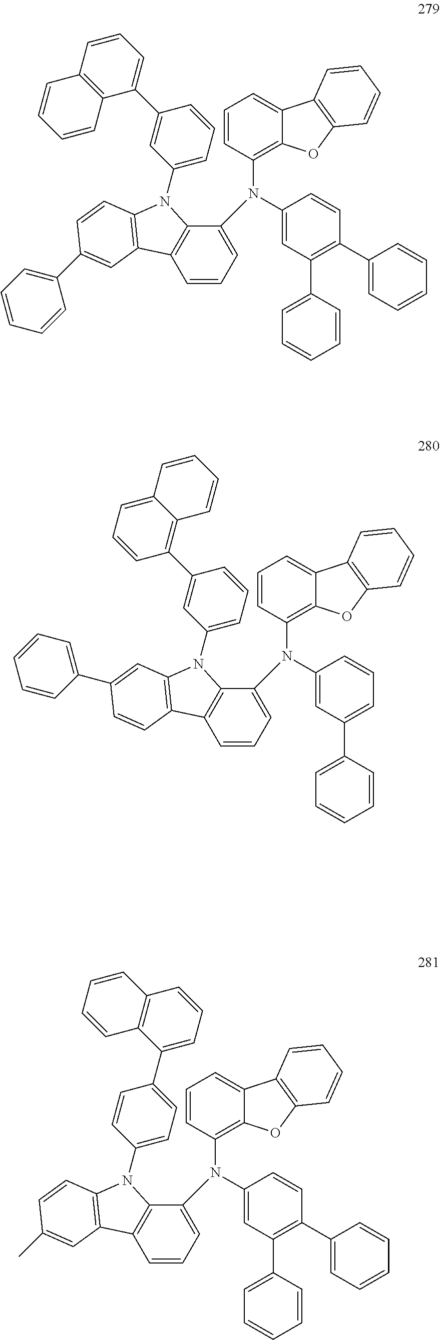 [SMILES: c1ccc(-c2ccc3c(c2)c2cccc(N(c4ccc(-c5ccccc5)c(-c5ccccc5)c4)c4cccc5c4oc4ccccc45)c2n3-c2cccc(-c3cccc4ccccc34)c2)cc1 ; c1ccc(-c2cccc(N(c3cccc4c3oc3ccccc34)c3cccc4c5ccc(-c6ccccc6)cc5n(-c5cccc(-c6cccc7ccccc67)c5)c34)c2)cc1 ; Cc1ccc2c(c1)c1cccc(N(c3ccc(-c4ccccc4)c(-c4ccccc4)c3)c3cccc4c3oc3ccccc34)c1n2-c1ccc(-c2cccc3ccccc23)cc1]

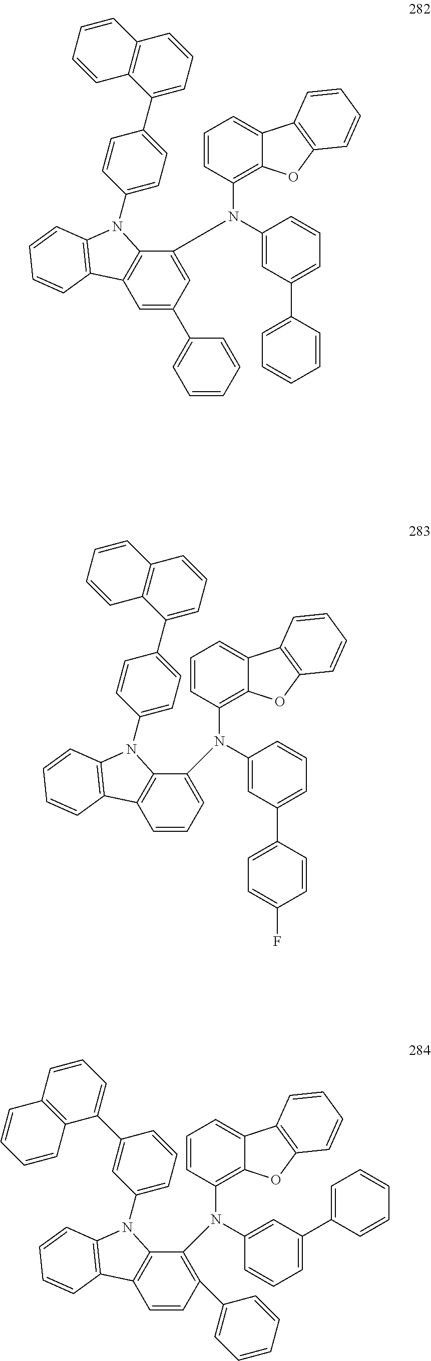 [SMILES: c1ccc(-c2cccc(N(c3cccc4c3oc3ccccc34)c3cc(-c4ccccc4)cc4c5ccccc5n(-c5ccc(-c6cccc7ccccc67)cc5)c34)c2)cc1 ; Fc1ccc(-c2cccc(N(c3cccc4c3oc3ccccc34)c3cccc4c5ccccc5n(-c5ccc(-c6cccc7ccccc67)cc5)c34)c2)cc1 ; c1ccc(-c2cccc(N(c3cccc4c3oc3ccccc34)c3c(-c4ccccc4)ccc4c5ccccc5n(-c5cccc(-c6cccc7ccccc67)c5)c34)c2)cc1]

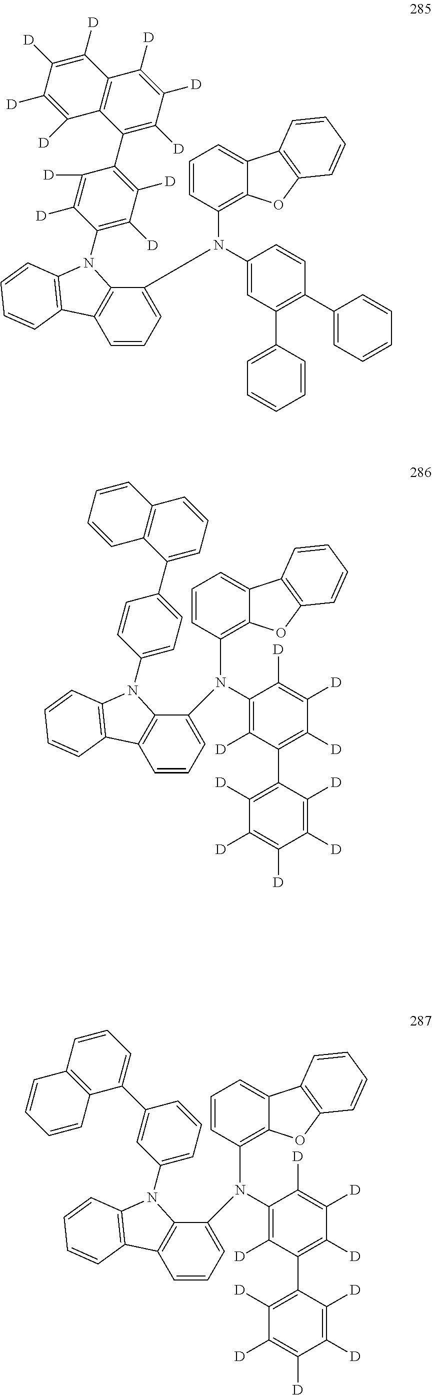 [SMILES: [2H]c1c([2H])c(-n2c3ccccc3c3cccc(N(c4ccc(-c5ccccc5)c(-c5ccccc5)c4)c4cccc5c4oc4ccccc45)c32)c([2H])c([2H])c1-c1c([2H])c([2H])c([2H])c2c([2H])c([2H])c([2H])c([2H])c12 ; [2H]c1c([2H])c([2H])c(-c2c([2H])c([2H])c([2H])c(N(c3cccc4c3oc3ccccc34)c3cccc4c5ccccc5n(-c5ccc(-c6cccc7ccccc67)cc5)c34)c2[2H])c([2H])c1[2H] ; [2H]c1c([2H])c([2H])c(-c2c([2H])c([2H])c([2H])c(N(c3cccc4c3oc3ccccc34)c3cccc4c5ccccc5n(-c5cccc(-c6cccc7ccccc67)c5)c34)c2[2H])c([2H])c1[2H]]

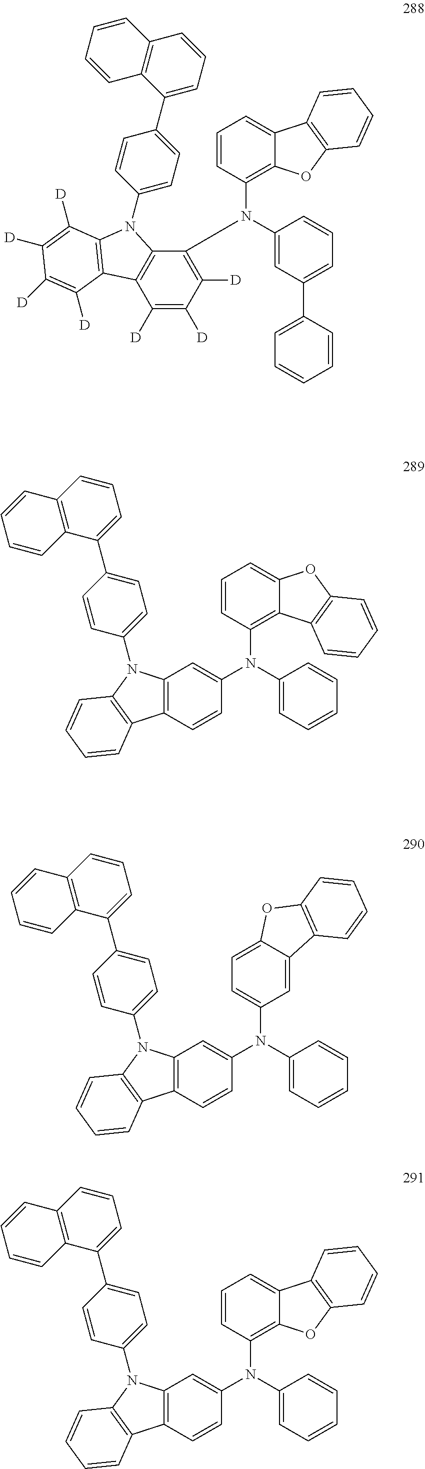 [SMILES: [2H]c1c([2H])c([2H])c2c(c1[2H])c1c([2H])c([2H])c([2H])c(N(c3cccc(-c4ccccc4)c3)c3cccc4c3oc3ccccc34)c1n2-c1ccc(-c2cccc3ccccc23)cc1 ; c1ccc(N(c2ccc3c4ccccc4n(-c4ccc(-c5cccc6ccccc56)cc4)c3c2)c2cccc3oc4ccccc4c23)cc1 ; c1ccc(N(c2ccc3oc4ccccc4c3c2)c2ccc3c4ccccc4n(-c4ccc(-c5cccc6ccccc56)cc4)c3c2)cc1 ; c1ccc(N(c2ccc3c4ccccc4n(-c4ccc(-c5cccc6ccccc56)cc4)c3c2)c2cccc3c2oc2ccccc23)cc1]

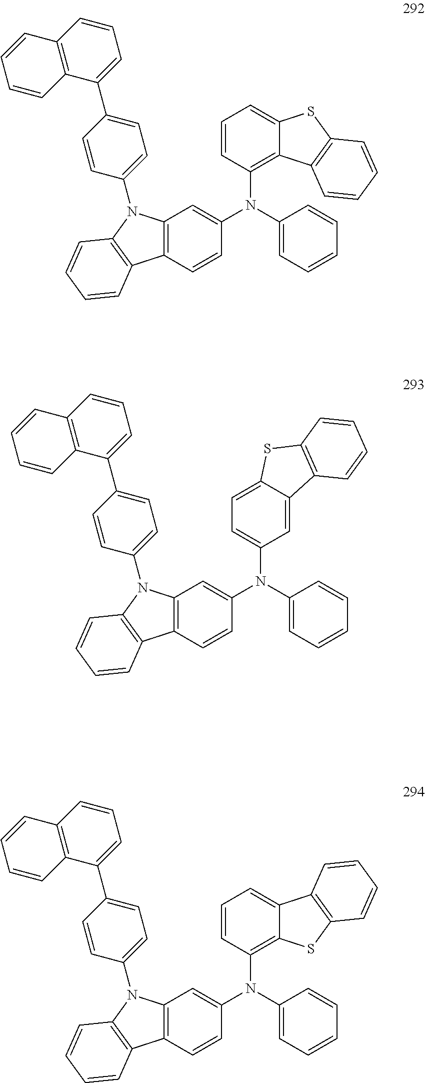 [SMILES: c1ccc(N(c2ccc3c4ccccc4n(-c4ccc(-c5cccc6ccccc56)cc4)c3c2)c2cccc3sc4ccccc4c23)cc1 ; c1ccc(N(c2ccc3sc4ccccc4c3c2)c2ccc3c4ccccc4n(-c4ccc(-c5cccc6ccccc56)cc4)c3c2)cc1 ; c1ccc(N(c2ccc3c4ccccc4n(-c4ccc(-c5cccc6ccccc56)cc4)c3c2)c2cccc3c2sc2ccccc23)cc1]

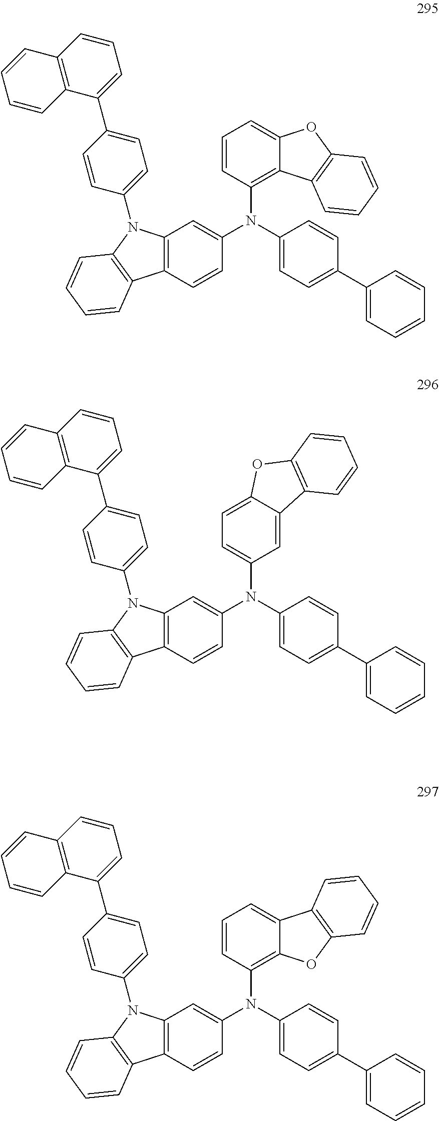 [SMILES: c1ccc(-c2ccc(N(c3ccc4c5ccccc5n(-c5ccc(-c6cccc7ccccc67)cc5)c4c3)c3cccc4oc5ccccc5c34)cc2)cc1 ; c1ccc(-c2ccc(N(c3ccc4oc5ccccc5c4c3)c3ccc4c5ccccc5n(-c5ccc(-c6cccc7ccccc67)cc5)c4c3)cc2)cc1 ; c1ccc(-c2ccc(N(c3ccc4c5ccccc5n(-c5ccc(-c6cccc7ccccc67)cc5)c4c3)c3cccc4c3oc3ccccc34)cc2)cc1]

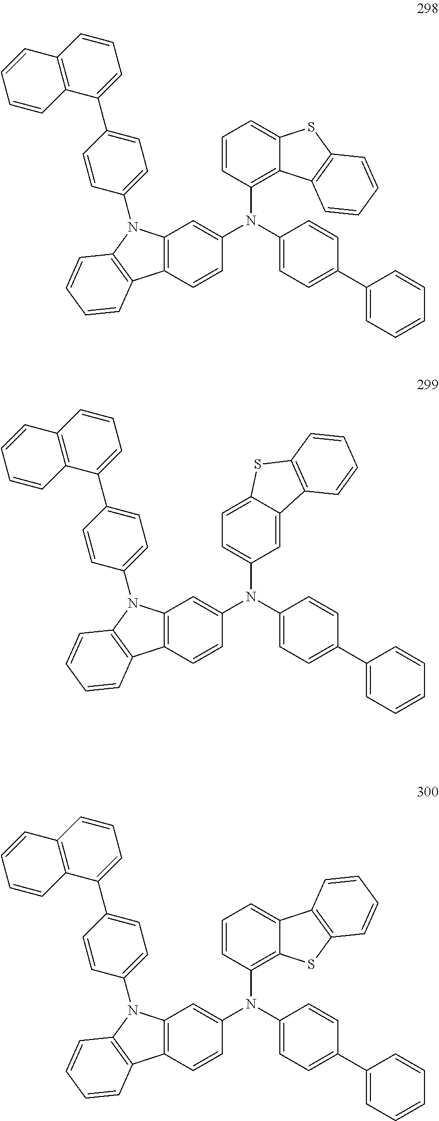 [SMILES: c1ccc(-c2ccc(N(c3ccc4c5ccccc5n(-c5ccc(-c6cccc7ccccc67)cc5)c4c3)c3cccc4sc5ccccc5c34)cc2)cc1 ; c1ccc(-c2ccc(N(c3ccc4sc5ccccc5c4c3)c3ccc4c5ccccc5n(-c5ccc(-c6cccc7ccccc67)cc5)c4c3)cc2)cc1 ; c1ccc(-c2ccc(N(c3ccc4c5ccccc5n(-c5ccc(-c6cccc7ccccc67)cc5)c4c3)c3cccc4c3sc3ccccc34)cc2)cc1]

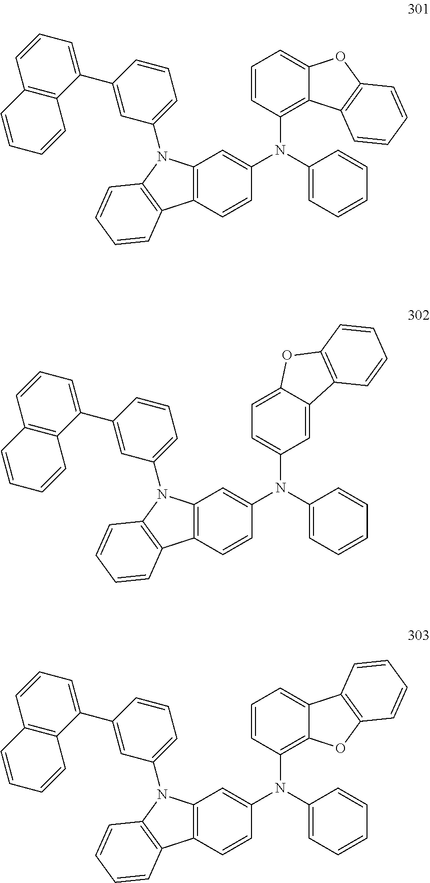 [SMILES: c1ccc(N(c2ccc3c4ccccc4n(-c4cccc(-c5cccc6ccccc56)c4)c3c2)c2cccc3oc4ccccc4c23)cc1 ; c1ccc(N(c2ccc3oc4ccccc4c3c2)c2ccc3c4ccccc4n(-c4cccc(-c5cccc6ccccc56)c4)c3c2)cc1 ; c1ccc(N(c2ccc3c4ccccc4n(-c4cccc(-c5cccc6ccccc56)c4)c3c2)c2cccc3c2oc2ccccc23)cc1]

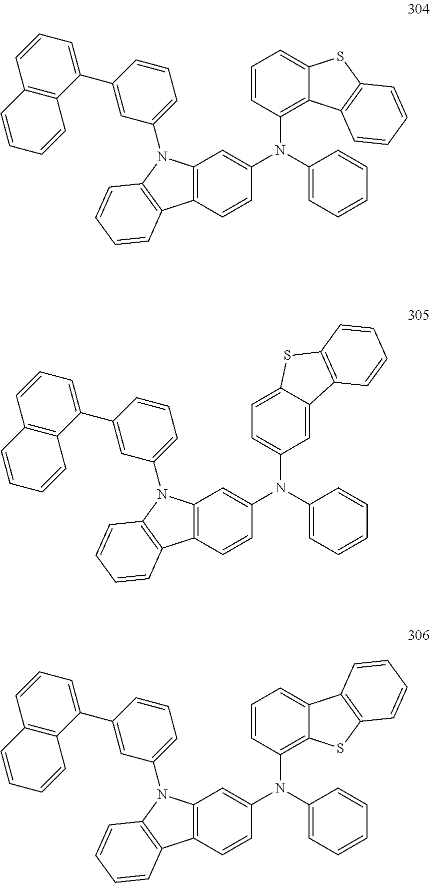 [SMILES: c1ccc(N(c2ccc3c4ccccc4n(-c4cccc(-c5cccc6ccccc56)c4)c3c2)c2cccc3sc4ccccc4c23)cc1 ; c1ccc(N(c2ccc3sc4ccccc4c3c2)c2ccc3c4ccccc4n(-c4cccc(-c5cccc6ccccc56)c4)c3c2)cc1 ; c1ccc(N(c2ccc3c4ccccc4n(-c4cccc(-c5cccc6ccccc56)c4)c3c2)c2cccc3c2sc2ccccc23)cc1]

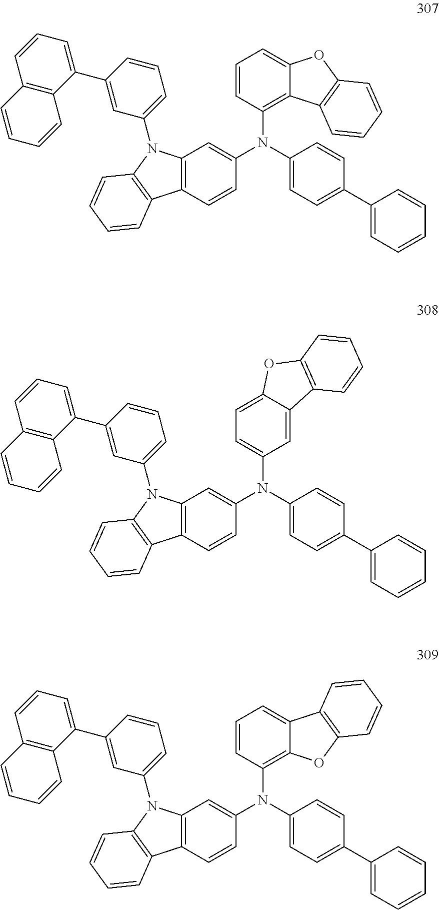 [SMILES: c1ccc(-c2ccc(N(c3ccc4c5ccccc5n(-c5cccc(-c6cccc7ccccc67)c5)c4c3)c3cccc4oc5ccccc5c34)cc2)cc1 ; c1ccc(-c2ccc(N(c3ccc4oc5ccccc5c4c3)c3ccc4c5ccccc5n(-c5cccc(-c6cccc7ccccc67)c5)c4c3)cc2)cc1 ; c1ccc(-c2ccc(N(c3ccc4c5ccccc5n(-c5cccc(-c6cccc7ccccc67)c5)c4c3)c3cccc4c3oc3ccccc34)cc2)cc1]

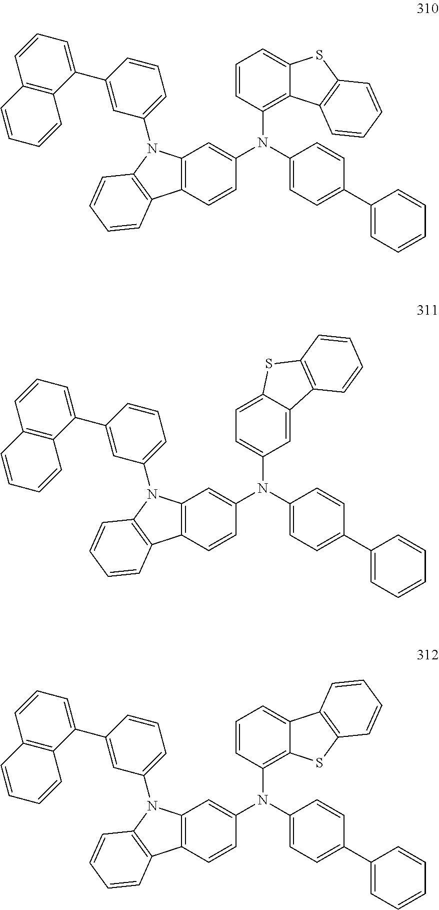 [SMILES: c1ccc(-c2ccc(N(c3ccc4c5ccccc5n(-c5cccc(-c6cccc7ccccc67)c5)c4c3)c3cccc4sc5ccccc5c34)cc2)cc1 ; c1ccc(-c2ccc(N(c3ccc4sc5ccccc5c4c3)c3ccc4c5ccccc5n(-c5cccc(-c6cccc7ccccc67)c5)c4c3)cc2)cc1 ; c1ccc(-c2ccc(N(c3ccc4c5ccccc5n(-c5cccc(-c6cccc7ccccc67)c5)c4c3)c3cccc4c3sc3ccccc34)cc2)cc1]

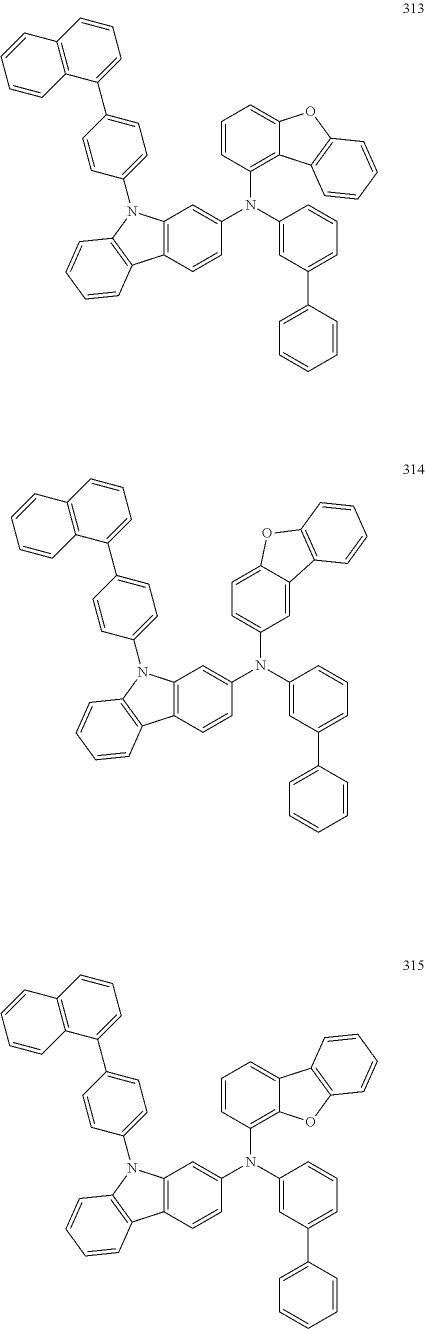 [SMILES: c1ccc(-c2cccc(N(c3ccc4c5ccccc5n(-c5ccc(-c6cccc7ccccc67)cc5)c4c3)c3cccc4oc5ccccc5c34)c2)cc1 ; c1ccc(-c2cccc(N(c3ccc4oc5ccccc5c4c3)c3ccc4c5ccccc5n(-c5ccc(-c6cccc7ccccc67)cc5)c4c3)c2)cc1 ; c1ccc(-c2cccc(N(c3ccc4c5ccccc5n(-c5ccc(-c6cccc7ccccc67)cc5)c4c3)c3cccc4c3oc3ccccc34)c2)cc1]

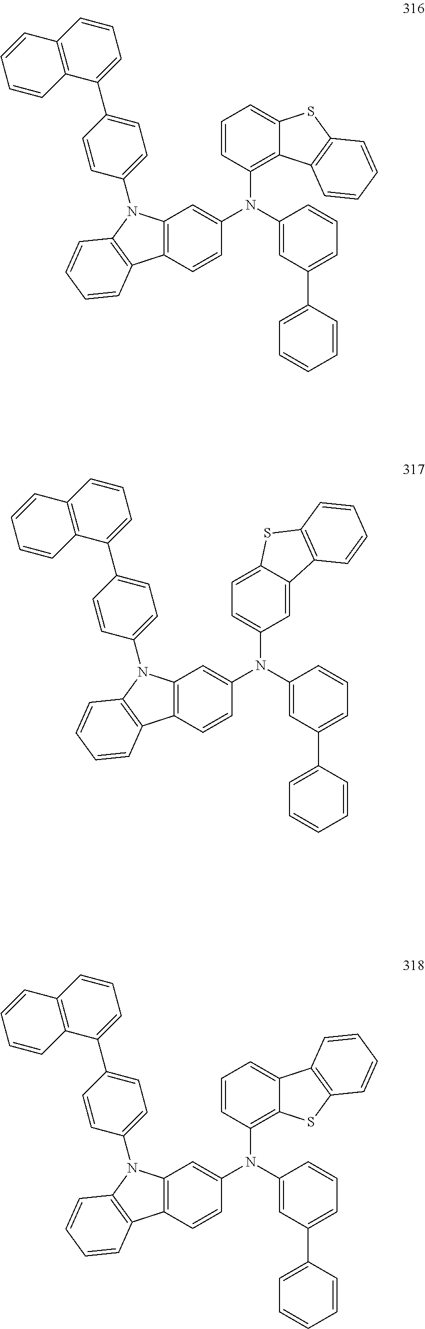 [SMILES: c1ccc(-c2cccc(N(c3ccc4c5ccccc5n(-c5ccc(-c6cccc7ccccc67)cc5)c4c3)c3cccc4sc5ccccc5c34)c2)cc1 ; c1ccc(-c2cccc(N(c3ccc4sc5ccccc5c4c3)c3ccc4c5ccccc5n(-c5ccc(-c6cccc7ccccc67)cc5)c4c3)c2)cc1 ; c1ccc(-c2cccc(N(c3ccc4c5ccccc5n(-c5ccc(-c6cccc7ccccc67)cc5)c4c3)c3cccc4c3sc3ccccc34)c2)cc1]

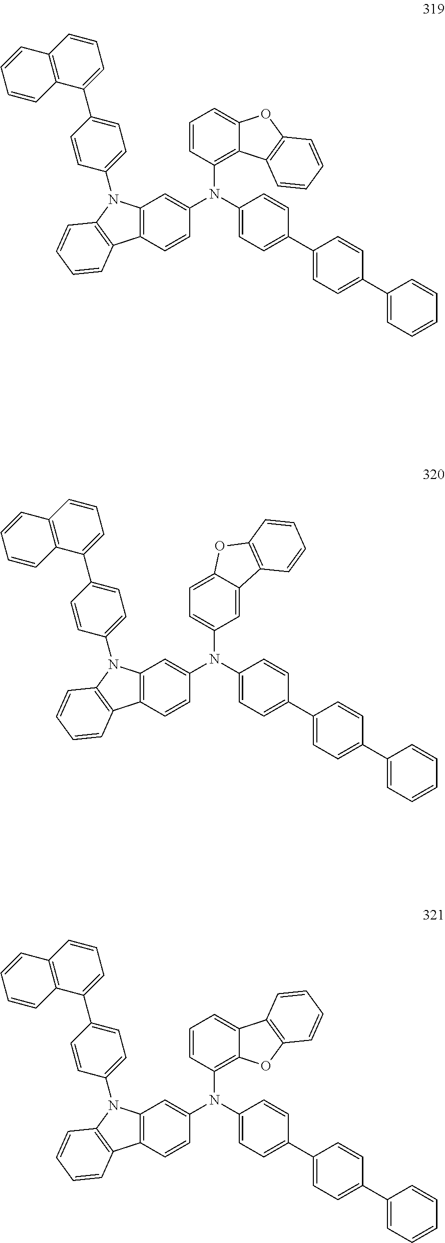 [SMILES: c1ccc(-c2ccc(-c3ccc(N(c4ccc5c6ccccc6n(-c6ccc(-c7cccc8ccccc78)cc6)c5c4)c4cccc5oc6ccccc6c45)cc3)cc2)cc1 ; c1ccc(-c2ccc(-c3ccc(N(c4ccc5oc6ccccc6c5c4)c4ccc5c6ccccc6n(-c6ccc(-c7cccc8ccccc78)cc6)c5c4)cc3)cc2)cc1 ; c1ccc(-c2ccc(-c3ccc(N(c4ccc5c6ccccc6n(-c6ccc(-c7cccc8ccccc78)cc6)c5c4)c4cccc5c4oc4ccccc45)cc3)cc2)cc1]

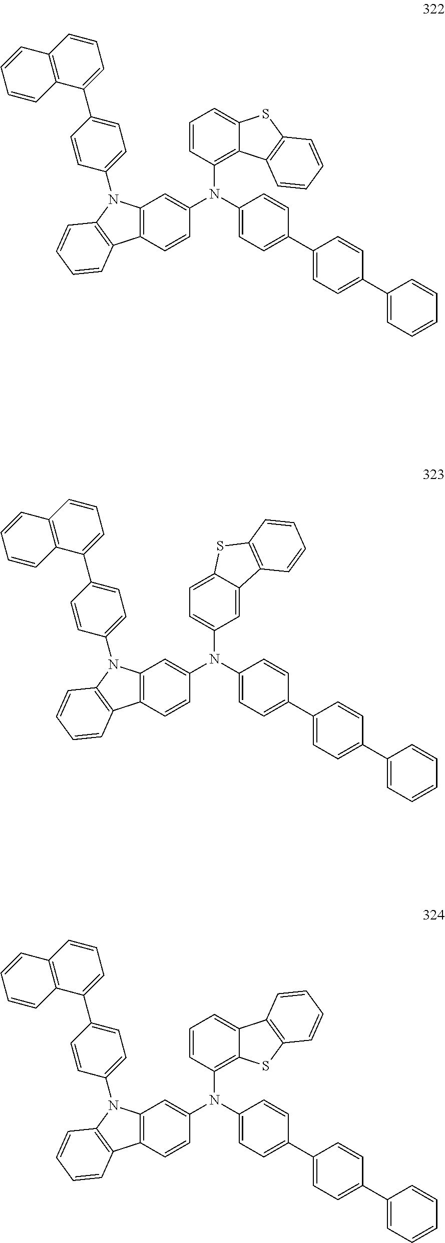 [SMILES: c1ccc(-c2ccc(-c3ccc(N(c4ccc5c6ccccc6n(-c6ccc(-c7cccc8ccccc78)cc6)c5c4)c4cccc5sc6ccccc6c45)cc3)cc2)cc1 ; c1ccc(-c2ccc(-c3ccc(N(c4ccc5sc6ccccc6c5c4)c4ccc5c6ccccc6n(-c6ccc(-c7cccc8ccccc78)cc6)c5c4)cc3)cc2)cc1 ; c1ccc(-c2ccc(-c3ccc(N(c4ccc5c6ccccc6n(-c6ccc(-c7cccc8ccccc78)cc6)c5c4)c4cccc5c4sc4ccccc45)cc3)cc2)cc1]

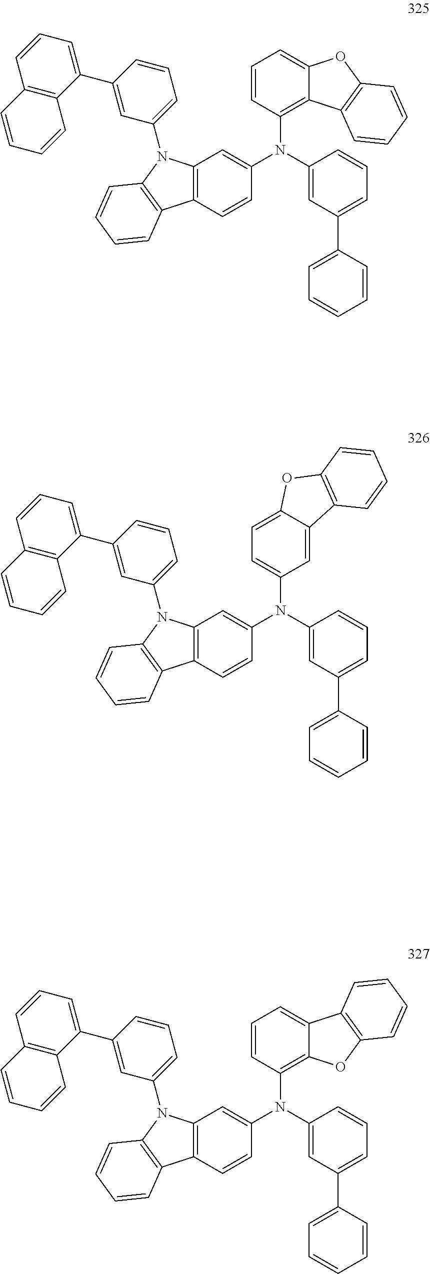 [SMILES: c1ccc(-c2cccc(N(c3ccc4c5ccccc5n(-c5cccc(-c6cccc7ccccc67)c5)c4c3)c3cccc4oc5ccccc5c34)c2)cc1 ; c1ccc(-c2cccc(N(c3ccc4oc5ccccc5c4c3)c3ccc4c5ccccc5n(-c5cccc(-c6cccc7ccccc67)c5)c4c3)c2)cc1 ; c1ccc(-c2cccc(N(c3ccc4c5ccccc5n(-c5cccc(-c6cccc7ccccc67)c5)c4c3)c3cccc4c3oc3ccccc34)c2)cc1]

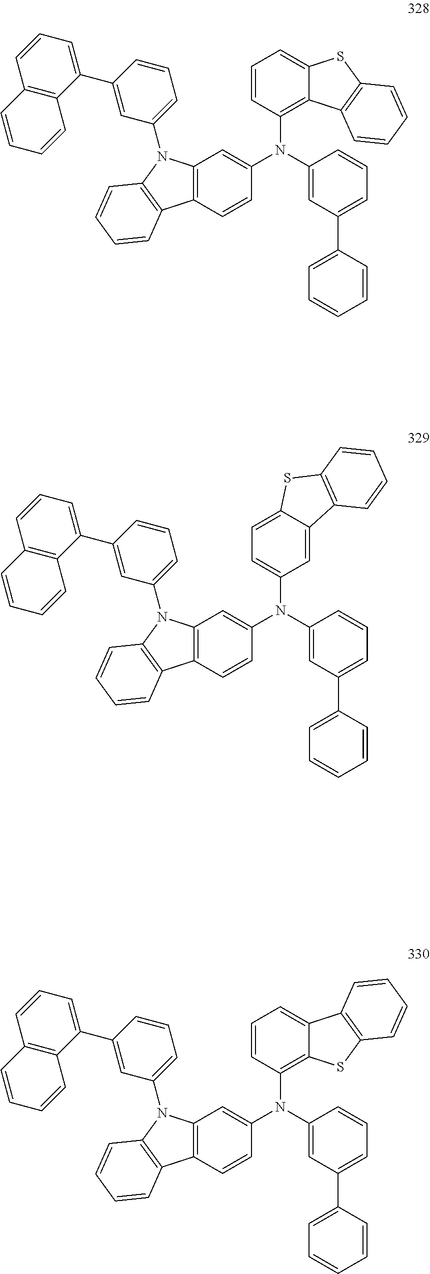 [SMILES: c1ccc(-c2cccc(N(c3ccc4c5ccccc5n(-c5cccc(-c6cccc7ccccc67)c5)c4c3)c3cccc4sc5ccccc5c34)c2)cc1 ; c1ccc(-c2cccc(N(c3ccc4sc5ccccc5c4c3)c3ccc4c5ccccc5n(-c5cccc(-c6cccc7ccccc67)c5)c4c3)c2)cc1 ; c1ccc(-c2cccc(N(c3ccc4c5ccccc5n(-c5cccc(-c6cccc7ccccc67)c5)c4c3)c3cccc4c3sc3ccccc34)c2)cc1]

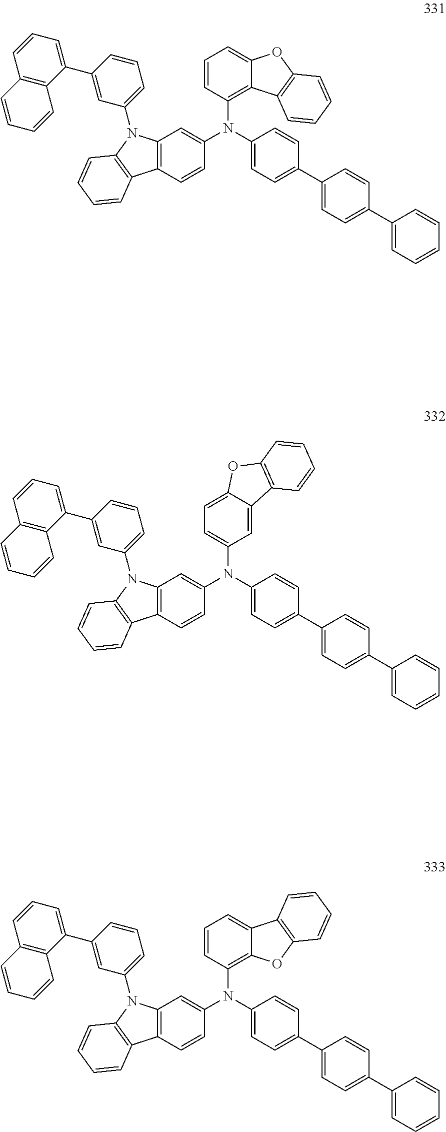 [SMILES: c1ccc(-c2ccc(-c3ccc(N(c4ccc5c6ccccc6n(-c6cccc(-c7cccc8ccccc78)c6)c5c4)c4cccc5oc6ccccc6c45)cc3)cc2)cc1 ; c1ccc(-c2ccc(-c3ccc(N(c4ccc5oc6ccccc6c5c4)c4ccc5c6ccccc6n(-c6cccc(-c7cccc8ccccc78)c6)c5c4)cc3)cc2)cc1 ; c1ccc(-c2ccc(-c3ccc(N(c4ccc5c6ccccc6n(-c6cccc(-c7cccc8ccccc78)c6)c5c4)c4cccc5c4oc4ccccc45)cc3)cc2)cc1]

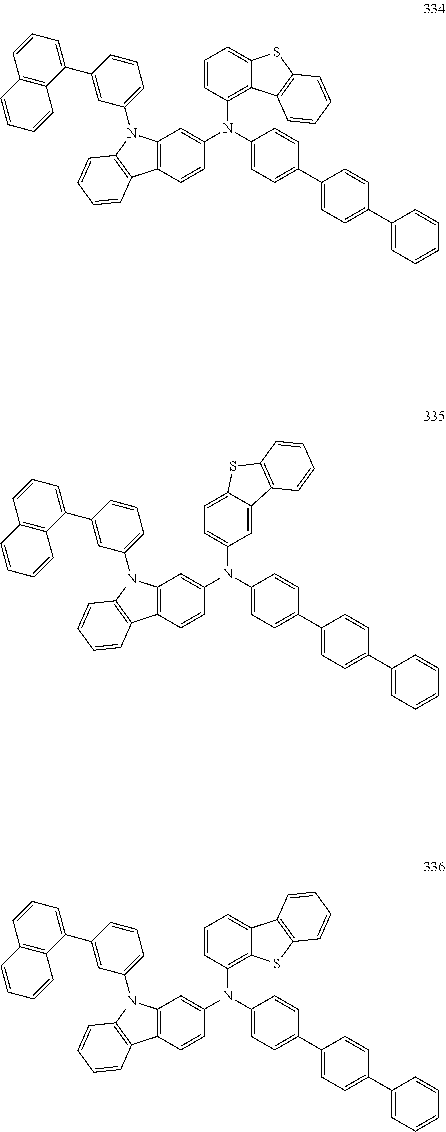 [SMILES: c1ccc(-c2ccc(-c3ccc(N(c4ccc5c6ccccc6n(-c6cccc(-c7cccc8ccccc78)c6)c5c4)c4cccc5sc6ccccc6c45)cc3)cc2)cc1 ; c1ccc(-c2ccc(-c3ccc(N(c4ccc5sc6ccccc6c5c4)c4ccc5c6ccccc6n(-c6cccc(-c7cccc8ccccc78)c6)c5c4)cc3)cc2)cc1 ; c1ccc(-c2ccc(-c3ccc(N(c4ccc5c6ccccc6n(-c6cccc(-c7cccc8ccccc78)c6)c5c4)c4cccc5c4sc4ccccc45)cc3)cc2)cc1]

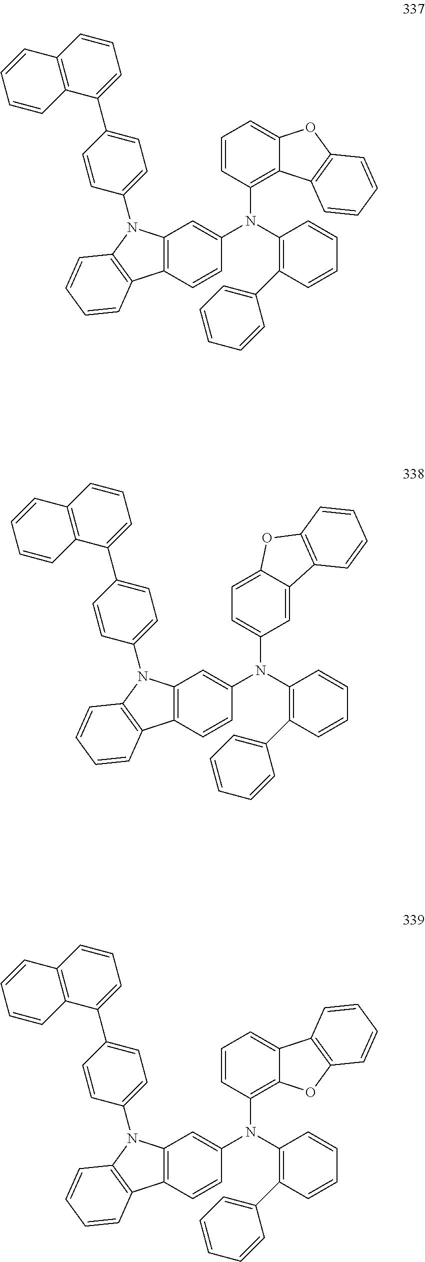 [SMILES: c1ccc(-c2ccccc2N(c2ccc3c4ccccc4n(-c4ccc(-c5cccc6ccccc56)cc4)c3c2)c2cccc3oc4ccccc4c23)cc1 ; c1ccc(-c2ccccc2N(c2ccc3oc4ccccc4c3c2)c2ccc3c4ccccc4n(-c4ccc(-c5cccc6ccccc56)cc4)c3c2)cc1 ; c1ccc(-c2ccccc2N(c2ccc3c4ccccc4n(-c4ccc(-c5cccc6ccccc56)cc4)c3c2)c2cccc3c2oc2ccccc23)cc1]

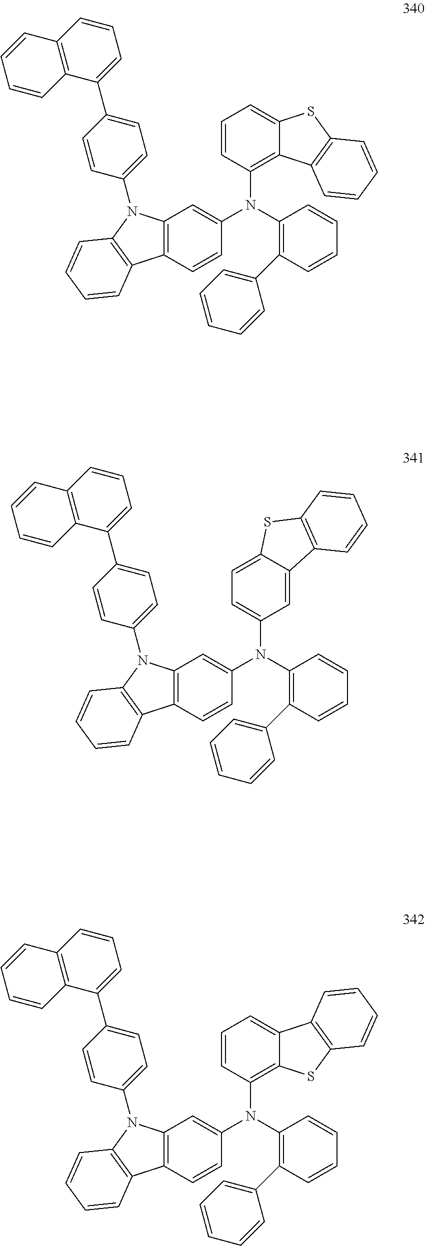 [SMILES: c1ccc(-c2ccccc2N(c2ccc3c4ccccc4n(-c4ccc(-c5cccc6ccccc56)cc4)c3c2)c2cccc3sc4ccccc4c23)cc1 ; c1ccc(-c2ccccc2N(c2ccc3sc4ccccc4c3c2)c2ccc3c4ccccc4n(-c4ccc(-c5cccc6ccccc56)cc4)c3c2)cc1 ; c1ccc(-c2ccccc2N(c2ccc3c4ccccc4n(-c4ccc(-c5cccc6ccccc56)cc4)c3c2)c2cccc3c2sc2ccccc23)cc1]

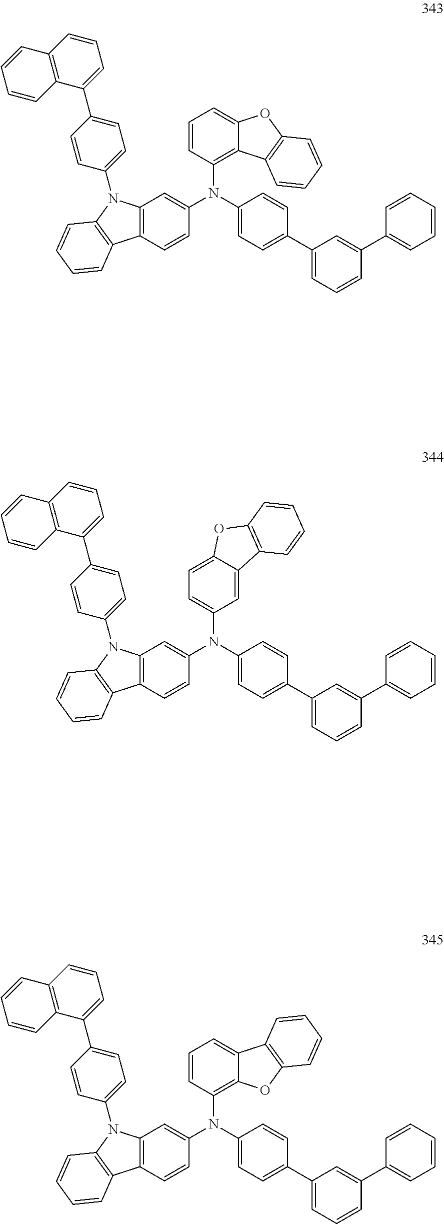 [SMILES: c1ccc(-c2cccc(-c3ccc(N(c4ccc5c6ccccc6n(-c6ccc(-c7cccc8ccccc78)cc6)c5c4)c4cccc5oc6ccccc6c45)cc3)c2)cc1 ; c1ccc(-c2cccc(-c3ccc(N(c4ccc5oc6ccccc6c5c4)c4ccc5c6ccccc6n(-c6ccc(-c7cccc8ccccc78)cc6)c5c4)cc3)c2)cc1 ; c1ccc(-c2cccc(-c3ccc(N(c4ccc5c6ccccc6n(-c6ccc(-c7cccc8ccccc78)cc6)c5c4)c4cccc5c4oc4ccccc45)cc3)c2)cc1]

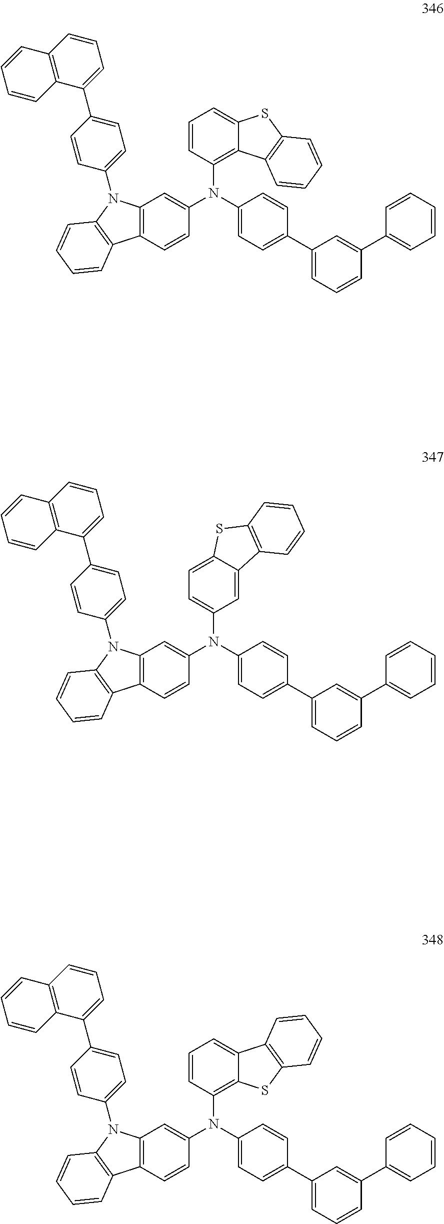 [SMILES: c1ccc(-c2cccc(-c3ccc(N(c4ccc5c6ccccc6n(-c6ccc(-c7cccc8ccccc78)cc6)c5c4)c4cccc5sc6ccccc6c45)cc3)c2)cc1 ; c1ccc(-c2cccc(-c3ccc(N(c4ccc5sc6ccccc6c5c4)c4ccc5c6ccccc6n(-c6ccc(-c7cccc8ccccc78)cc6)c5c4)cc3)c2)cc1 ; c1ccc(-c2cccc(-c3ccc(N(c4ccc5c6ccccc6n(-c6ccc(-c7cccc8ccccc78)cc6)c5c4)c4cccc5c4sc4ccccc45)cc3)c2)cc1]

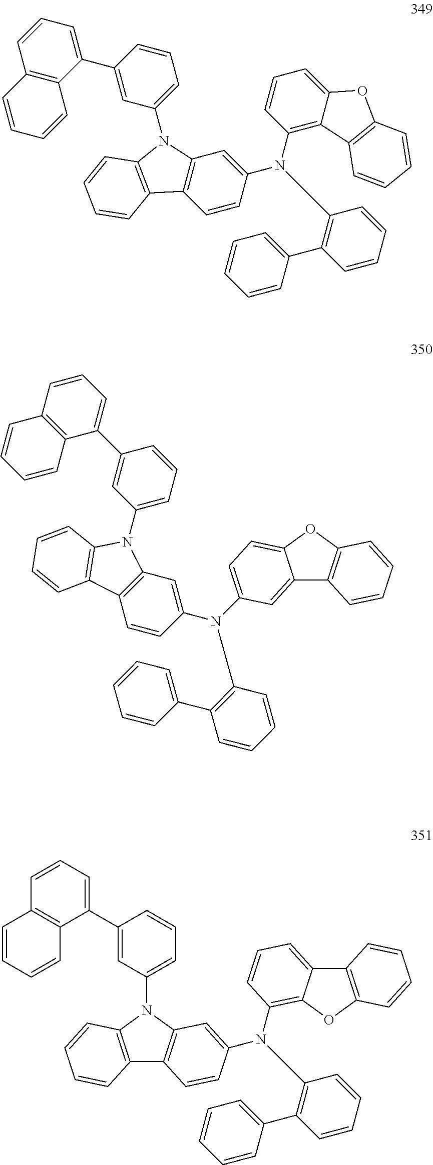 [SMILES: c1ccc(-c2ccccc2N(c2ccc3c4ccccc4n(-c4cccc(-c5cccc6ccccc56)c4)c3c2)c2cccc3oc4ccccc4c23)cc1 ; c1ccc(-c2ccccc2N(c2ccc3oc4ccccc4c3c2)c2ccc3c4ccccc4n(-c4cccc(-c5cccc6ccccc56)c4)c3c2)cc1 ; c1ccc(-c2ccccc2N(c2ccc3c4ccccc4n(-c4cccc(-c5cccc6ccccc56)c4)c3c2)c2cccc3c2oc2ccccc23)cc1]

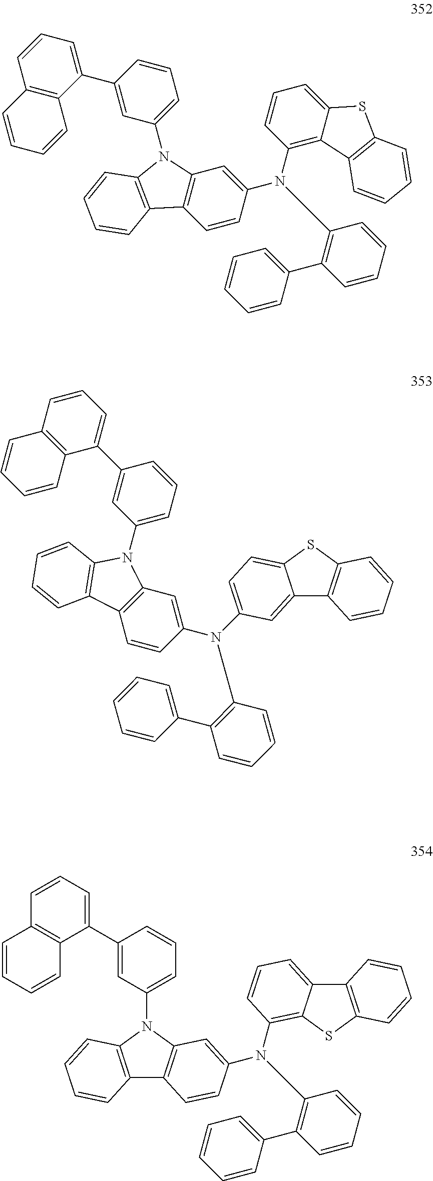 [SMILES: c1ccc(-c2ccccc2N(c2ccc3c4ccccc4n(-c4cccc(-c5cccc6ccccc56)c4)c3c2)c2cccc3sc4ccccc4c23)cc1 ; c1ccc(-c2ccccc2N(c2ccc3sc4ccccc4c3c2)c2ccc3c4ccccc4n(-c4cccc(-c5cccc6ccccc56)c4)c3c2)cc1 ; c1ccc(-c2ccccc2N(c2ccc3c4ccccc4n(-c4cccc(-c5cccc6ccccc56)c4)c3c2)c2cccc3c2sc2ccccc23)cc1]

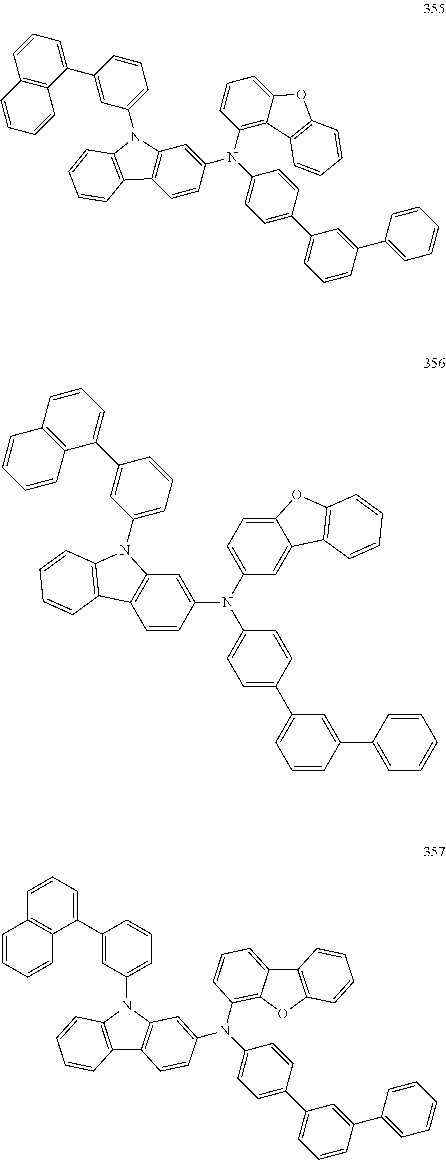 [SMILES: c1ccc(-c2cccc(-c3ccc(N(c4ccc5c6ccccc6n(-c6cccc(-c7cccc8ccccc78)c6)c5c4)c4cccc5oc6ccccc6c45)cc3)c2)cc1 ; c1ccc(-c2cccc(-c3ccc(N(c4ccc5oc6ccccc6c5c4)c4ccc5c6ccccc6n(-c6cccc(-c7cccc8ccccc78)c6)c5c4)cc3)c2)cc1 ; c1ccc(-c2cccc(-c3ccc(N(c4ccc5c6ccccc6n(-c6cccc(-c7cccc8ccccc78)c6)c5c4)c4cccc5c4oc4ccccc45)cc3)c2)cc1]

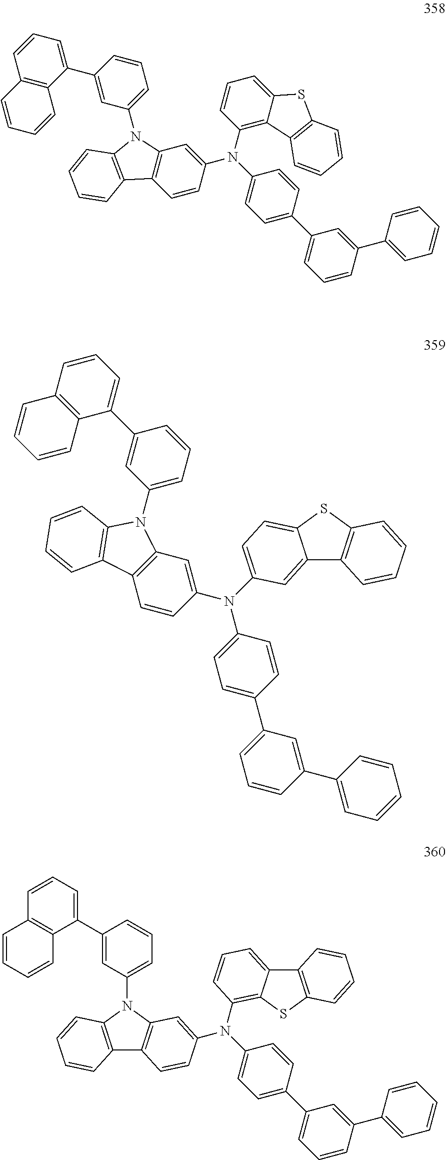 [SMILES: c1ccc(-c2cccc(-c3ccc(N(c4ccc5c6ccccc6n(-c6cccc(-c7cccc8ccccc78)c6)c5c4)c4cccc5sc6ccccc6c45)cc3)c2)cc1 ; c1ccc(-c2cccc(-c3ccc(N(c4ccc5sc6ccccc6c5c4)c4ccc5c6ccccc6n(-c6cccc(-c7cccc8ccccc78)c6)c5c4)cc3)c2)cc1 ; c1ccc(-c2cccc(-c3ccc(N(c4ccc5c6ccccc6n(-c6cccc(-c7cccc8ccccc78)c6)c5c4)c4cccc5c4sc4ccccc45)cc3)c2)cc1]

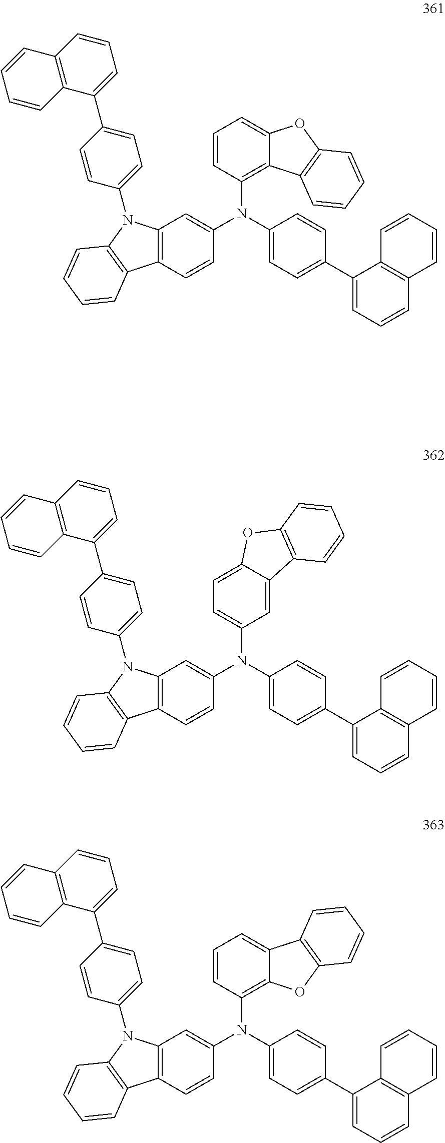 [SMILES: c1ccc2c(-c3ccc(N(c4ccc5c6ccccc6n(-c6ccc(-c7cccc8ccccc78)cc6)c5c4)c4cccc5oc6ccccc6c45)cc3)cccc2c1 ; c1ccc2c(-c3ccc(N(c4ccc5oc6ccccc6c5c4)c4ccc5c6ccccc6n(-c6ccc(-c7cccc8ccccc78)cc6)c5c4)cc3)cccc2c1 ; c1ccc2c(-c3ccc(N(c4ccc5c6ccccc6n(-c6ccc(-c7cccc8ccccc78)cc6)c5c4)c4cccc5c4oc4ccccc45)cc3)cccc2c1]

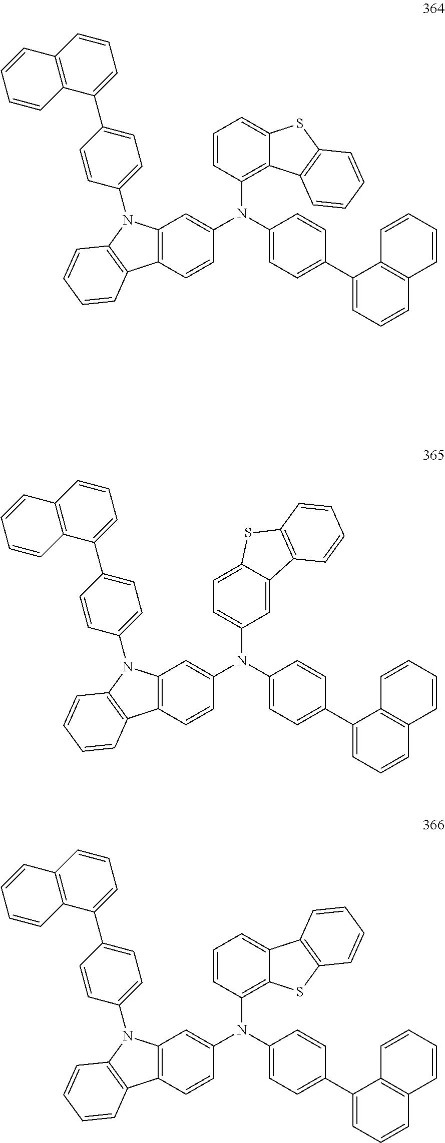 [SMILES: c1ccc2c(-c3ccc(N(c4ccc5c6ccccc6n(-c6ccc(-c7cccc8ccccc78)cc6)c5c4)c4cccc5sc6ccccc6c45)cc3)cccc2c1 ; c1ccc2c(-c3ccc(N(c4ccc5sc6ccccc6c5c4)c4ccc5c6ccccc6n(-c6ccc(-c7cccc8ccccc78)cc6)c5c4)cc3)cccc2c1 ; c1ccc2c(-c3ccc(N(c4ccc5c6ccccc6n(-c6ccc(-c7cccc8ccccc78)cc6)c5c4)c4cccc5c4sc4ccccc45)cc3)cccc2c1]

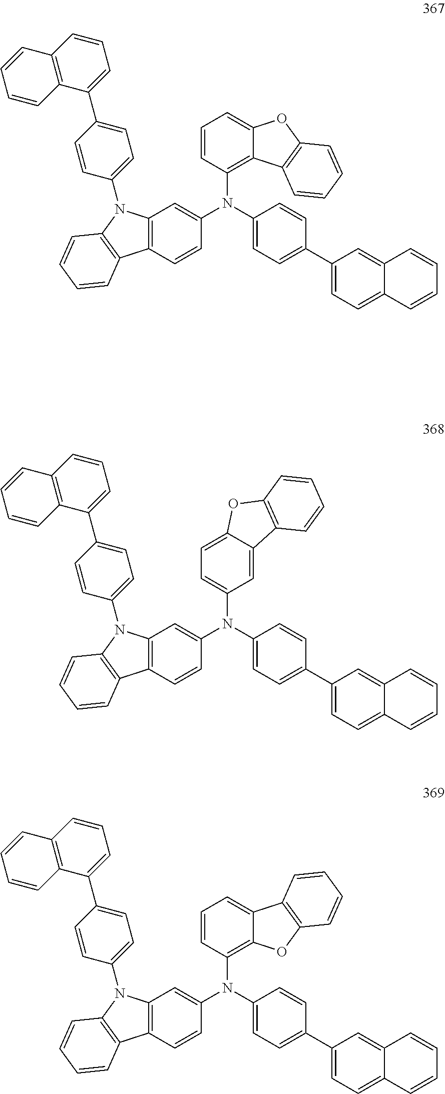 [SMILES: c1ccc2cc(-c3ccc(N(c4ccc5c6ccccc6n(-c6ccc(-c7cccc8ccccc78)cc6)c5c4)c4cccc5oc6ccccc6c45)cc3)ccc2c1 ; c1ccc2cc(-c3ccc(N(c4ccc5oc6ccccc6c5c4)c4ccc5c6ccccc6n(-c6ccc(-c7cccc8ccccc78)cc6)c5c4)cc3)ccc2c1 ; c1ccc2cc(-c3ccc(N(c4ccc5c6ccccc6n(-c6ccc(-c7cccc8ccccc78)cc6)c5c4)c4cccc5c4oc4ccccc45)cc3)ccc2c1]

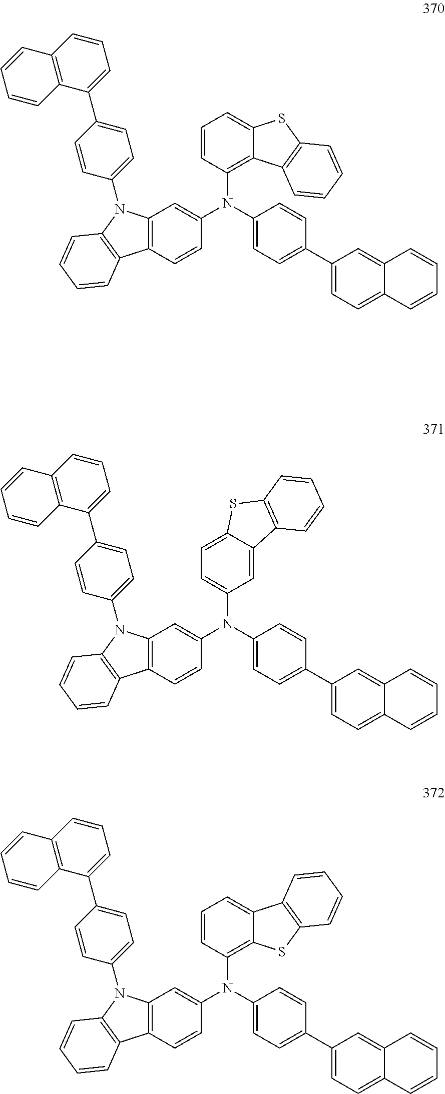 [SMILES: c1ccc2cc(-c3ccc(N(c4ccc5c6ccccc6n(-c6ccc(-c7cccc8ccccc78)cc6)c5c4)c4cccc5sc6ccccc6c45)cc3)ccc2c1 ; c1ccc2cc(-c3ccc(N(c4ccc5sc6ccccc6c5c4)c4ccc5c6ccccc6n(-c6ccc(-c7cccc8ccccc78)cc6)c5c4)cc3)ccc2c1 ; c1ccc2cc(-c3ccc(N(c4ccc5c6ccccc6n(-c6ccc(-c7cccc8ccccc78)cc6)c5c4)c4cccc5c4sc4ccccc45)cc3)ccc2c1]

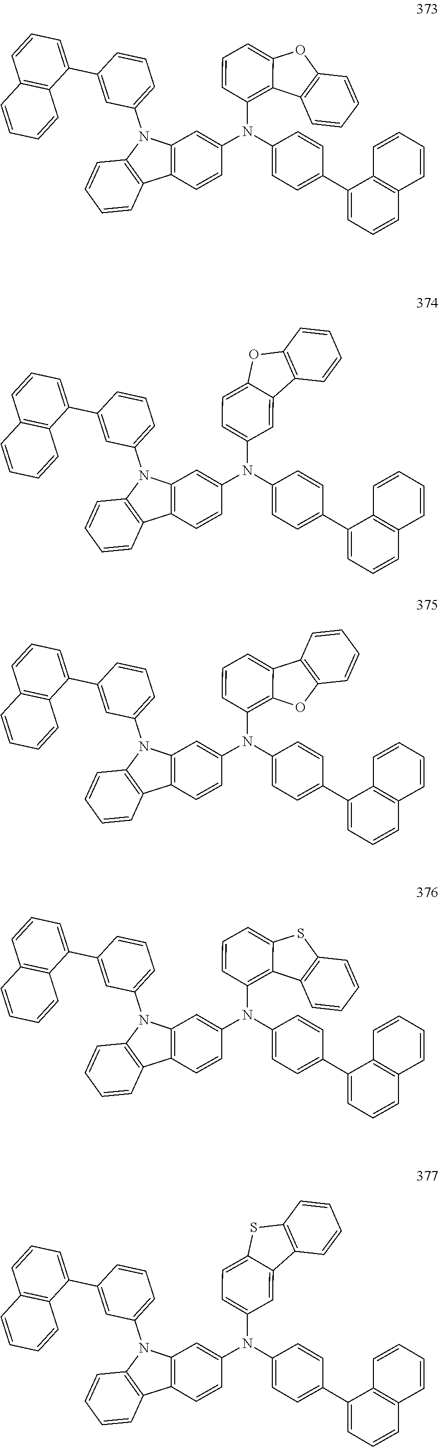 [SMILES: c1cc(-c2cccc3ccccc23)cc(-n2c3ccccc3c3ccc(N(c4ccc(-c5cccc6ccccc56)cc4)c4cccc5oc6ccccc6c45)cc32)c1 ; c1cc(-c2cccc3ccccc23)cc(-n2c3ccccc3c3ccc(N(c4ccc(-c5cccc6ccccc56)cc4)c4ccc5oc6ccccc6c5c4)cc32)c1 ; c1cc(-c2cccc3ccccc23)cc(-n2c3ccccc3c3ccc(N(c4ccc(-c5cccc6ccccc56)cc4)c4cccc5c4oc4ccccc45)cc32)c1 ; c1cc(-c2cccc3ccccc23)cc(-n2c3ccccc3c3ccc(N(c4ccc(-c5cccc6ccccc56)cc4)c4cccc5sc6ccccc6c45)cc32)c1 ; c1cc(-c2cccc3ccccc23)cc(-n2c3ccccc3c3ccc(N(c4ccc(-c5cccc6ccccc56)cc4)c4ccc5sc6ccccc6c5c4)cc32)c1]

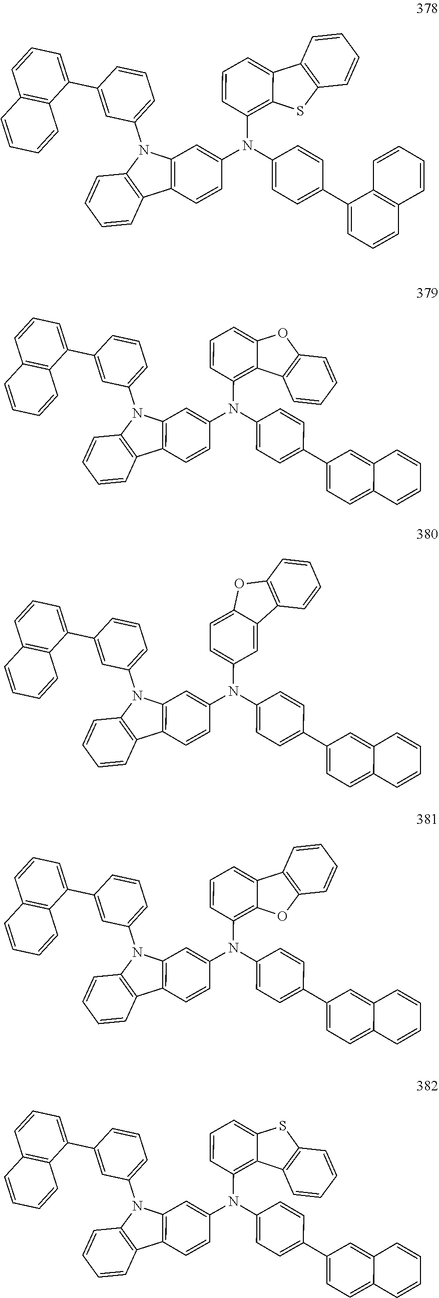 [SMILES: c1cc(-c2cccc3ccccc23)cc(-n2c3ccccc3c3ccc(N(c4ccc(-c5cccc6ccccc56)cc4)c4cccc5c4sc4ccccc45)cc32)c1 ; c1cc(-c2cccc3ccccc23)cc(-n2c3ccccc3c3ccc(N(c4ccc(-c5ccc6ccccc6c5)cc4)c4cccc5oc6ccccc6c45)cc32)c1 ; c1cc(-c2cccc3ccccc23)cc(-n2c3ccccc3c3ccc(N(c4ccc(-c5ccc6ccccc6c5)cc4)c4ccc5oc6ccccc6c5c4)cc32)c1 ; c1cc(-c2cccc3ccccc23)cc(-n2c3ccccc3c3ccc(N(c4ccc(-c5ccc6ccccc6c5)cc4)c4cccc5c4oc4ccccc45)cc32)c1 ; c1cc(-c2cccc3ccccc23)cc(-n2c3ccccc3c3ccc(N(c4ccc(-c5ccc6ccccc6c5)cc4)c4cccc5sc6ccccc6c45)cc32)c1]

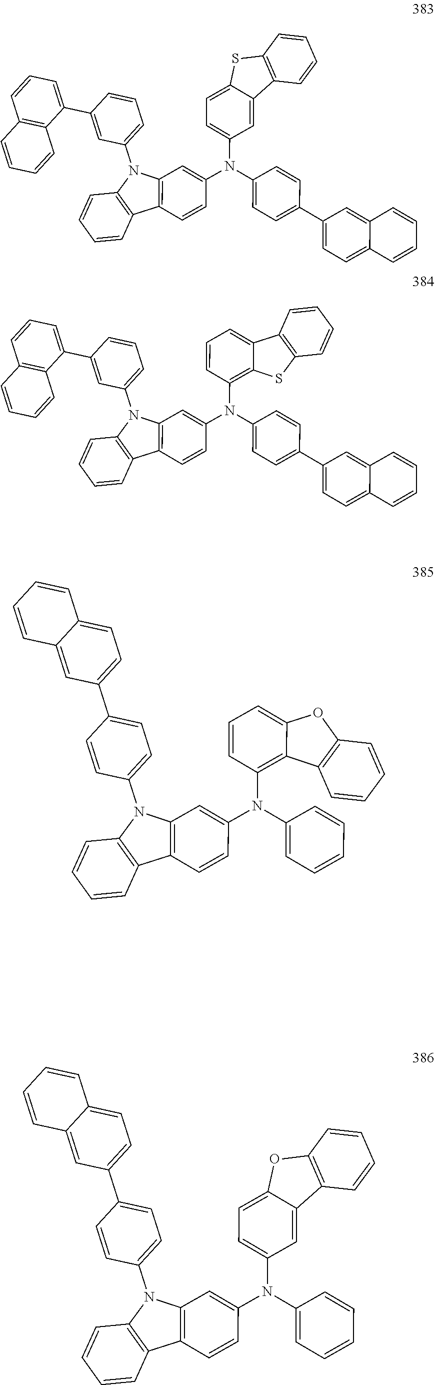 [SMILES: c1cc(-c2cccc3ccccc23)cc(-n2c3ccccc3c3ccc(N(c4ccc(-c5ccc6ccccc6c5)cc4)c4ccc5sc6ccccc6c5c4)cc32)c1 ; c1cc(-c2cccc3ccccc23)cc(-n2c3ccccc3c3ccc(N(c4ccc(-c5ccc6ccccc6c5)cc4)c4cccc5c4sc4ccccc45)cc32)c1 ; c1ccc(N(c2ccc3c4ccccc4n(-c4ccc(-c5ccc6ccccc6c5)cc4)c3c2)c2cccc3oc4ccccc4c23)cc1 ; c1ccc(N(c2ccc3oc4ccccc4c3c2)c2ccc3c4ccccc4n(-c4ccc(-c5ccc6ccccc6c5)cc4)c3c2)cc1]

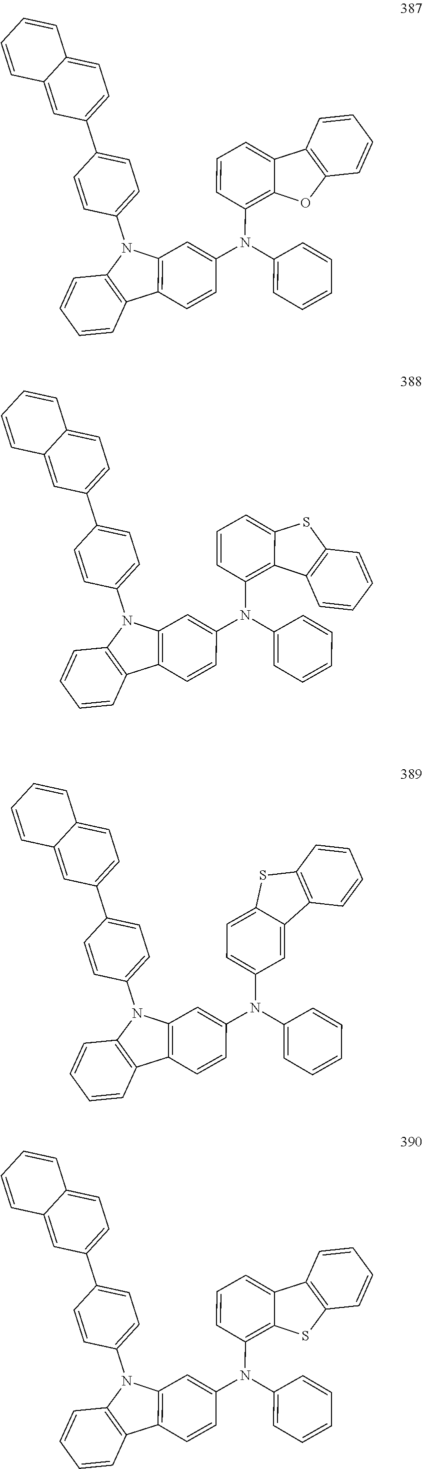 [SMILES: c1ccc(N(c2ccc3c4ccccc4n(-c4ccc(-c5ccc6ccccc6c5)cc4)c3c2)c2cccc3c2oc2ccccc23)cc1 ; c1ccc(N(c2ccc3c4ccccc4n(-c4ccc(-c5ccc6ccccc6c5)cc4)c3c2)c2cccc3sc4ccccc4c23)cc1 ; c1ccc(N(c2ccc3sc4ccccc4c3c2)c2ccc3c4ccccc4n(-c4ccc(-c5ccc6ccccc6c5)cc4)c3c2)cc1 ; c1ccc(N(c2ccc3c4ccccc4n(-c4ccc(-c5ccc6ccccc6c5)cc4)c3c2)c2cccc3c2sc2ccccc23)cc1]

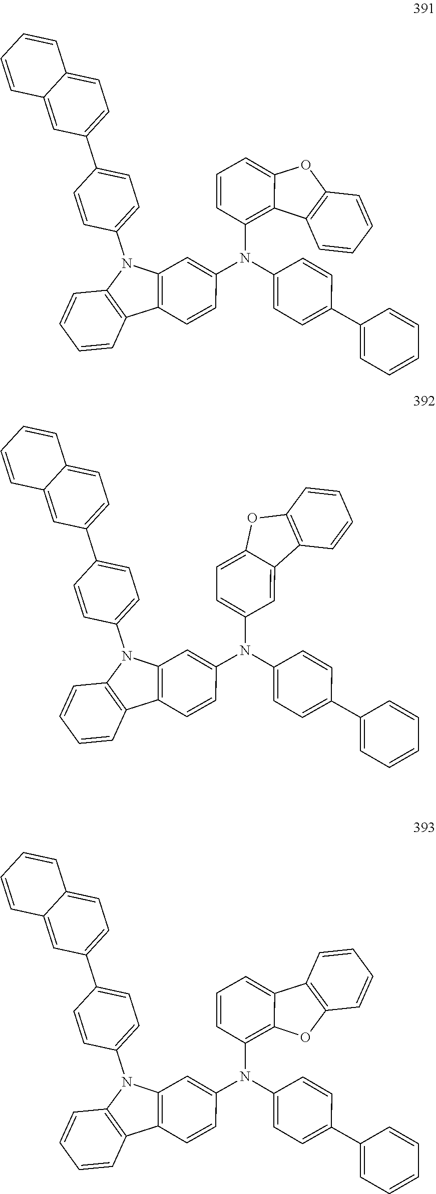 [SMILES: c1ccc(-c2ccc(N(c3ccc4c5ccccc5n(-c5ccc(-c6ccc7ccccc7c6)cc5)c4c3)c3cccc4oc5ccccc5c34)cc2)cc1 ; c1ccc(-c2ccc(N(c3ccc4oc5ccccc5c4c3)c3ccc4c5ccccc5n(-c5ccc(-c6ccc7ccccc7c6)cc5)c4c3)cc2)cc1 ; c1ccc(-c2ccc(N(c3ccc4c5ccccc5n(-c5ccc(-c6ccc7ccccc7c6)cc5)c4c3)c3cccc4c3oc3ccccc34)cc2)cc1]

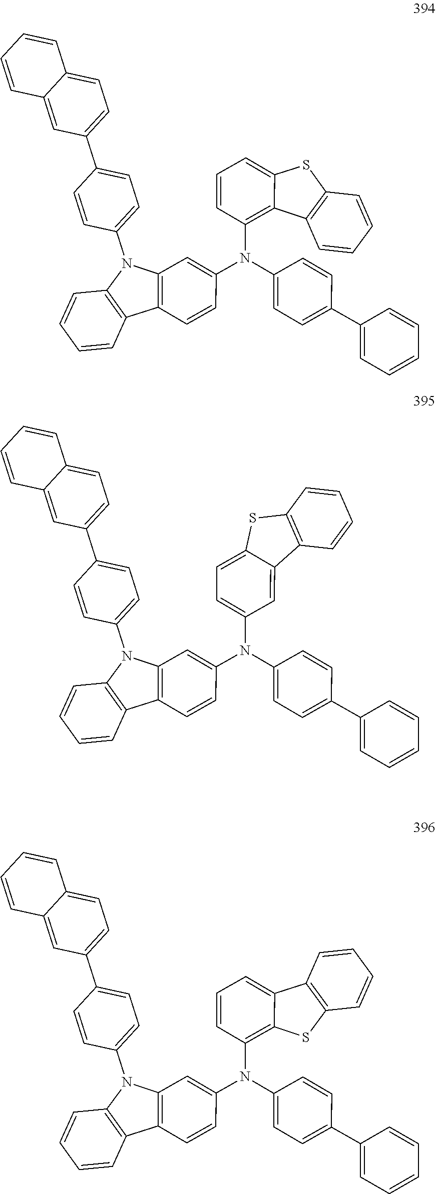 [SMILES: c1ccc(-c2ccc(N(c3ccc4c5ccccc5n(-c5ccc(-c6ccc7ccccc7c6)cc5)c4c3)c3cccc4sc5ccccc5c34)cc2)cc1 ; c1ccc(-c2ccc(N(c3ccc4sc5ccccc5c4c3)c3ccc4c5ccccc5n(-c5ccc(-c6ccc7ccccc7c6)cc5)c4c3)cc2)cc1 ; c1ccc(-c2ccc(N(c3ccc4c5ccccc5n(-c5ccc(-c6ccc7ccccc7c6)cc5)c4c3)c3cccc4c3sc3ccccc34)cc2)cc1]

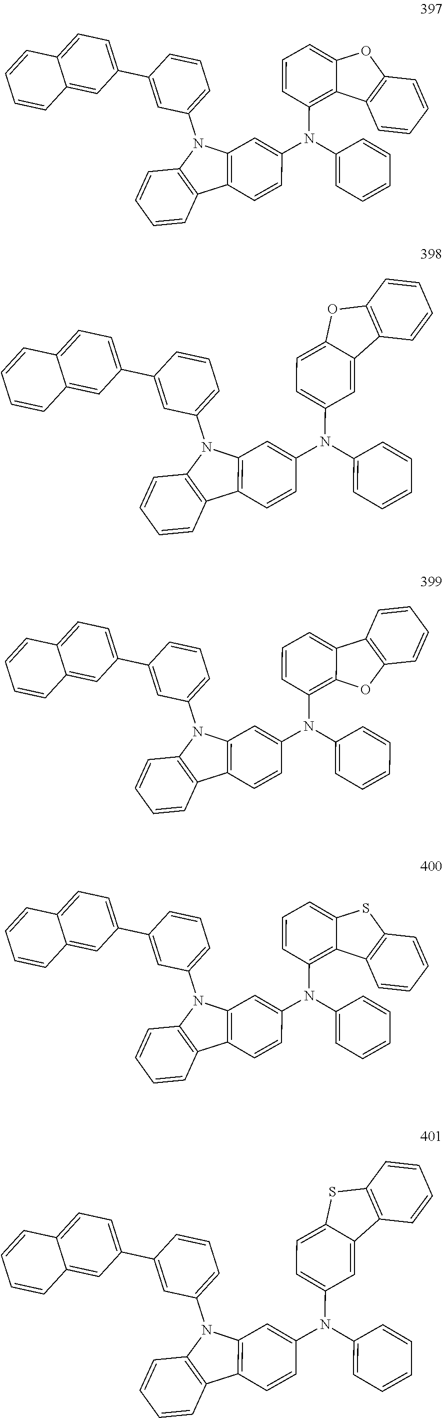 [SMILES: c1ccc(N(c2ccc3c4ccccc4n(-c4cccc(-c5ccc6ccccc6c5)c4)c3c2)c2cccc3oc4ccccc4c23)cc1 ; c1ccc(N(c2ccc3oc4ccccc4c3c2)c2ccc3c4ccccc4n(-c4cccc(-c5ccc6ccccc6c5)c4)c3c2)cc1 ; c1ccc(N(c2ccc3c4ccccc4n(-c4cccc(-c5ccc6ccccc6c5)c4)c3c2)c2cccc3c2oc2ccccc23)cc1 ; c1ccc(N(c2ccc3c4ccccc4n(-c4cccc(-c5ccc6ccccc6c5)c4)c3c2)c2cccc3sc4ccccc4c23)cc1 ; c1ccc(N(c2ccc3sc4ccccc4c3c2)c2ccc3c4ccccc4n(-c4cccc(-c5ccc6ccccc6c5)c4)c3c2)cc1]

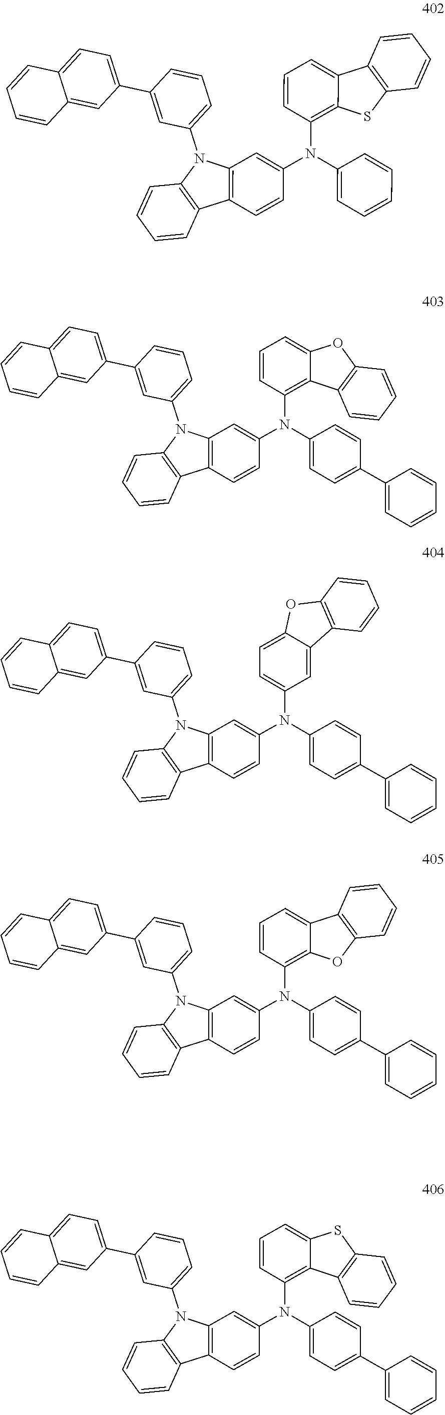 [SMILES: c1ccc(N(c2ccc3c4ccccc4n(-c4cccc(-c5ccc6ccccc6c5)c4)c3c2)c2cccc3c2sc2ccccc23)cc1 ; c1ccc(-c2ccc(N(c3ccc4c5ccccc5n(-c5cccc(-c6ccc7ccccc7c6)c5)c4c3)c3cccc4oc5ccccc5c34)cc2)cc1 ; c1ccc(-c2ccc(N(c3ccc4oc5ccccc5c4c3)c3ccc4c5ccccc5n(-c5cccc(-c6ccc7ccccc7c6)c5)c4c3)cc2)cc1 ; c1ccc(-c2ccc(N(c3ccc4c5ccccc5n(-c5cccc(-c6ccc7ccccc7c6)c5)c4c3)c3cccc4c3oc3ccccc34)cc2)cc1 ; c1ccc(-c2ccc(N(c3ccc4c5ccccc5n(-c5cccc(-c6ccc7ccccc7c6)c5)c4c3)c3cccc4sc5ccccc5c34)cc2)cc1]

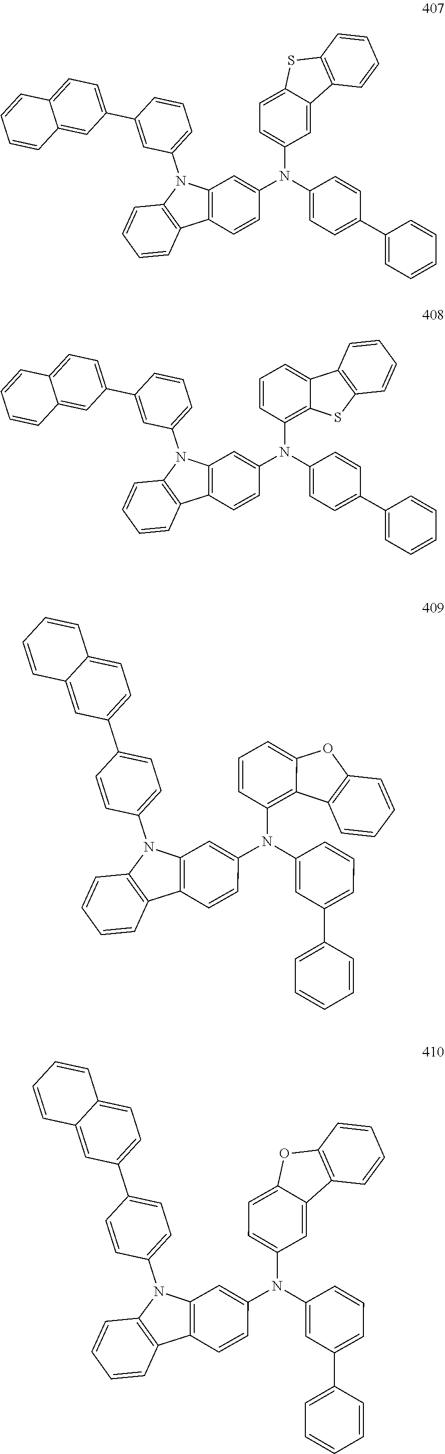 [SMILES: c1ccc(-c2ccc(N(c3ccc4sc5ccccc5c4c3)c3ccc4c5ccccc5n(-c5cccc(-c6ccc7ccccc7c6)c5)c4c3)cc2)cc1 ; c1ccc(-c2ccc(N(c3ccc4c5ccccc5n(-c5cccc(-c6ccc7ccccc7c6)c5)c4c3)c3cccc4c3sc3ccccc34)cc2)cc1 ; c1ccc(-c2cccc(N(c3ccc4c5ccccc5n(-c5ccc(-c6ccc7ccccc7c6)cc5)c4c3)c3cccc4oc5ccccc5c34)c2)cc1 ; c1ccc(-c2cccc(N(c3ccc4oc5ccccc5c4c3)c3ccc4c5ccccc5n(-c5ccc(-c6ccc7ccccc7c6)cc5)c4c3)c2)cc1]

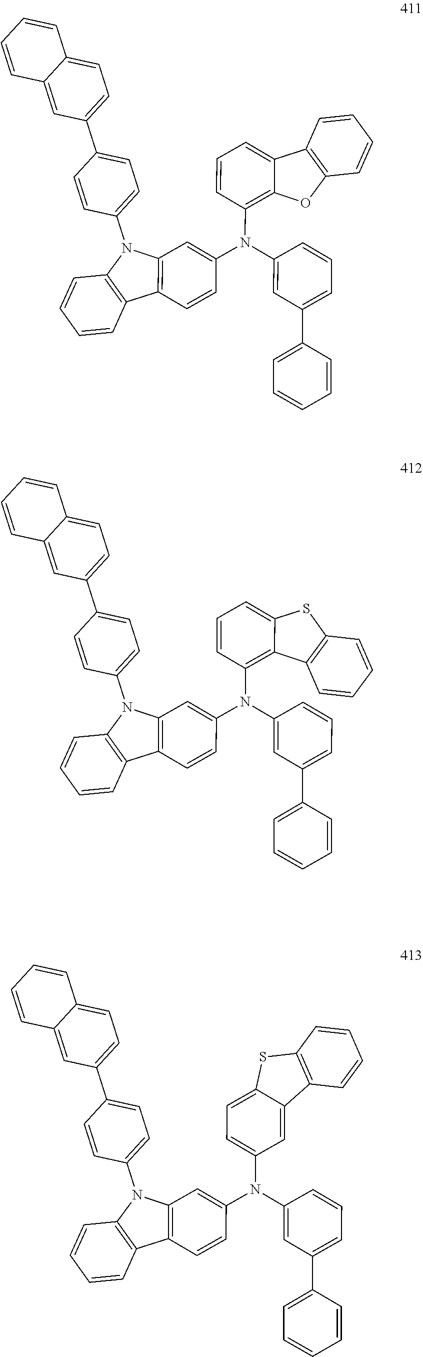 [SMILES: c1ccc(-c2cccc(N(c3ccc4c5ccccc5n(-c5ccc(-c6ccc7ccccc7c6)cc5)c4c3)c3cccc4c3oc3ccccc34)c2)cc1 ; c1ccc(-c2cccc(N(c3ccc4c5ccccc5n(-c5ccc(-c6ccc7ccccc7c6)cc5)c4c3)c3cccc4sc5ccccc5c34)c2)cc1 ; c1ccc(-c2cccc(N(c3ccc4sc5ccccc5c4c3)c3ccc4c5ccccc5n(-c5ccc(-c6ccc7ccccc7c6)cc5)c4c3)c2)cc1]

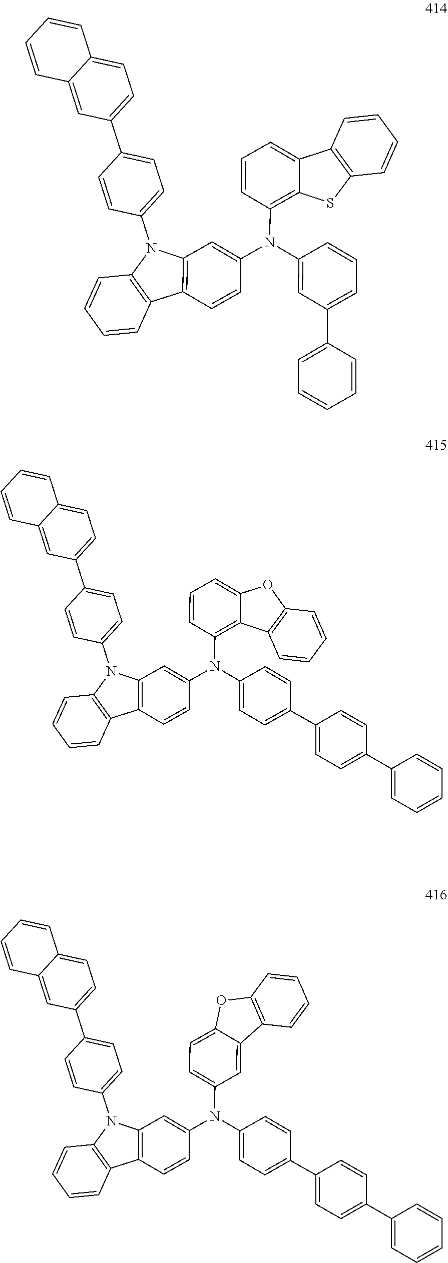 [SMILES: c1ccc(-c2cccc(N(c3ccc4c5ccccc5n(-c5ccc(-c6ccc7ccccc7c6)cc5)c4c3)c3cccc4c3sc3ccccc34)c2)cc1 ; c1ccc(-c2ccc(-c3ccc(N(c4ccc5c6ccccc6n(-c6ccc(-c7ccc8ccccc8c7)cc6)c5c4)c4cccc5oc6ccccc6c45)cc3)cc2)cc1 ; c1ccc(-c2ccc(-c3ccc(N(c4ccc5oc6ccccc6c5c4)c4ccc5c6ccccc6n(-c6ccc(-c7ccc8ccccc8c7)cc6)c5c4)cc3)cc2)cc1]

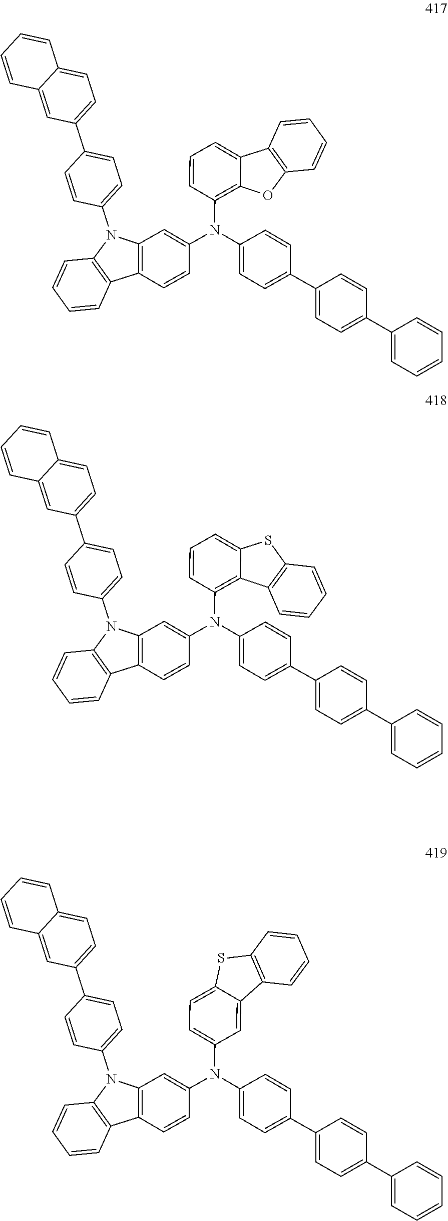 [SMILES: c1ccc(-c2ccc(-c3ccc(N(c4ccc5c6ccccc6n(-c6ccc(-c7ccc8ccccc8c7)cc6)c5c4)c4cccc5c4oc4ccccc45)cc3)cc2)cc1 ; c1ccc(-c2ccc(-c3ccc(N(c4ccc5c6ccccc6n(-c6ccc(-c7ccc8ccccc8c7)cc6)c5c4)c4cccc5sc6ccccc6c45)cc3)cc2)cc1 ; c1ccc(-c2ccc(-c3ccc(N(c4ccc5sc6ccccc6c5c4)c4ccc5c6ccccc6n(-c6ccc(-c7ccc8ccccc8c7)cc6)c5c4)cc3)cc2)cc1]

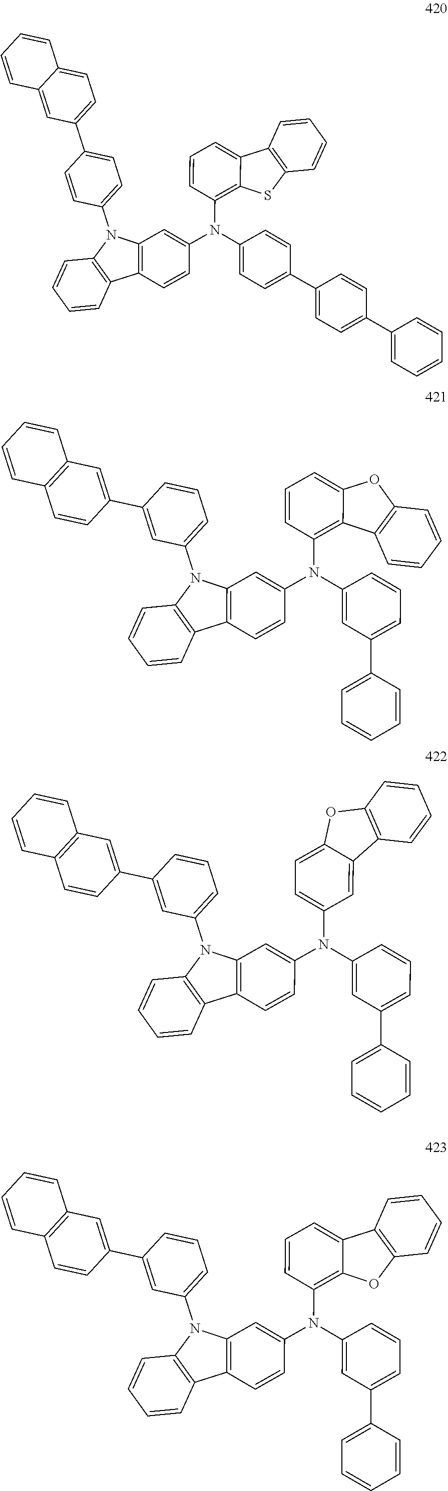

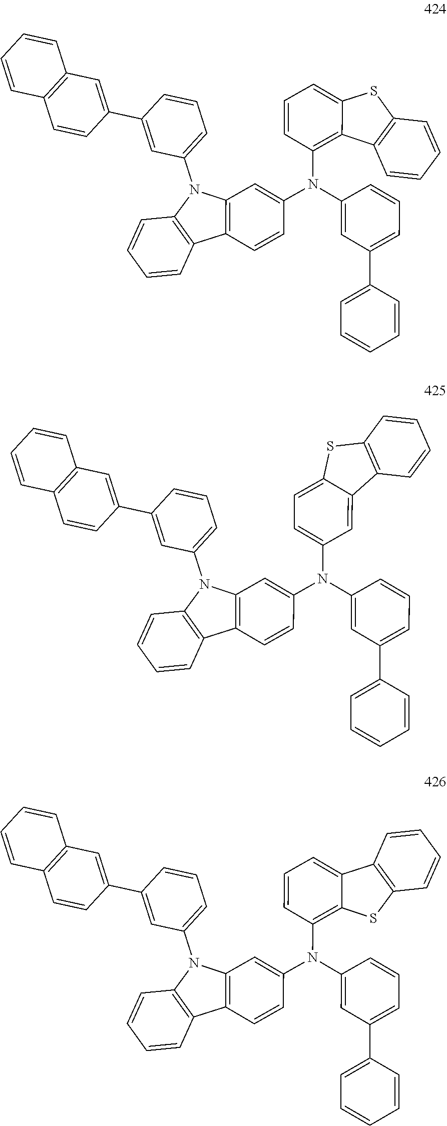 [SMILES: c1ccc(-c2cccc(N(c3ccc4c5ccccc5n(-c5cccc(-c6ccc7ccccc7c6)c5)c4c3)c3cccc4sc5ccccc5c34)c2)cc1 ; c1ccc(-c2cccc(N(c3ccc4sc5ccccc5c4c3)c3ccc4c5ccccc5n(-c5cccc(-c6ccc7ccccc7c6)c5)c4c3)c2)cc1 ; c1ccc(-c2cccc(N(c3ccc4c5ccccc5n(-c5cccc(-c6ccc7ccccc7c6)c5)c4c3)c3cccc4c3sc3ccccc34)c2)cc1]

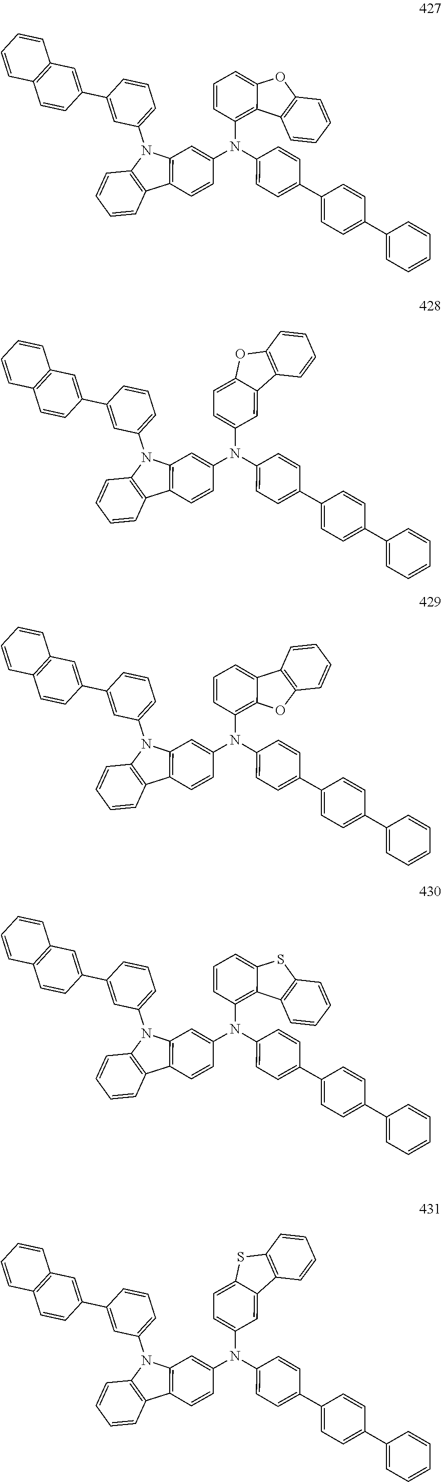

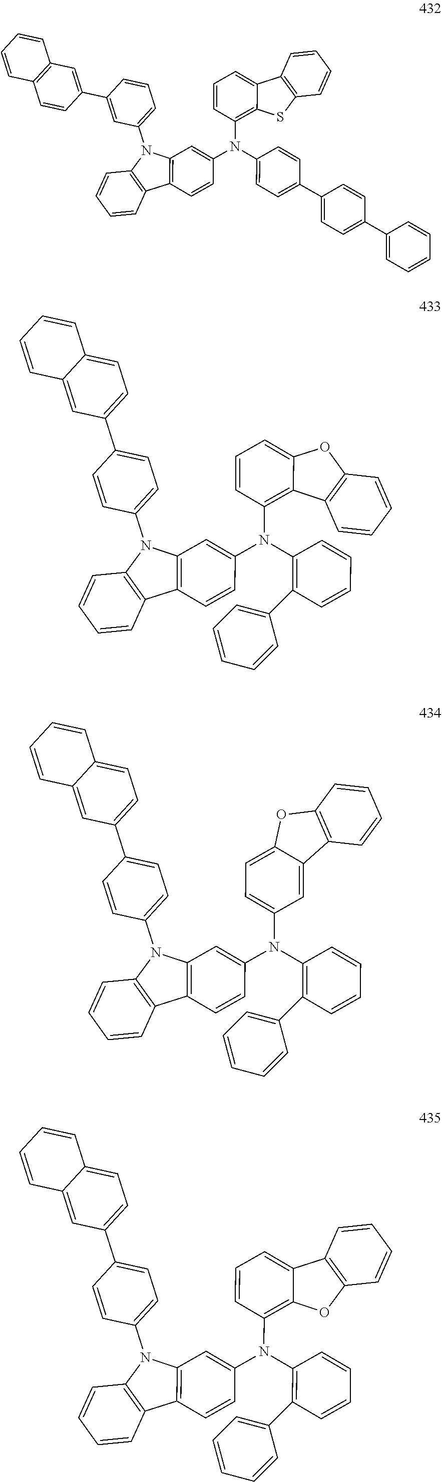 [SMILES: c1ccc(-c2ccc(-c3ccc(N(c4ccc5c6ccccc6n(-c6cccc(-c7ccc8ccccc8c7)c6)c5c4)c4cccc5c4sc4ccccc45)cc3)cc2)cc1 ; c1ccc(-c2ccccc2N(c2ccc3c4ccccc4n(-c4ccc(-c5ccc6ccccc6c5)cc4)c3c2)c2cccc3oc4ccccc4c23)cc1 ; c1ccc(-c2ccccc2N(c2ccc3oc4ccccc4c3c2)c2ccc3c4ccccc4n(-c4ccc(-c5ccc6ccccc6c5)cc4)c3c2)cc1 ; c1ccc(-c2ccccc2N(c2ccc3c4ccccc4n(-c4ccc(-c5ccc6ccccc6c5)cc4)c3c2)c2cccc3c2oc2ccccc23)cc1]

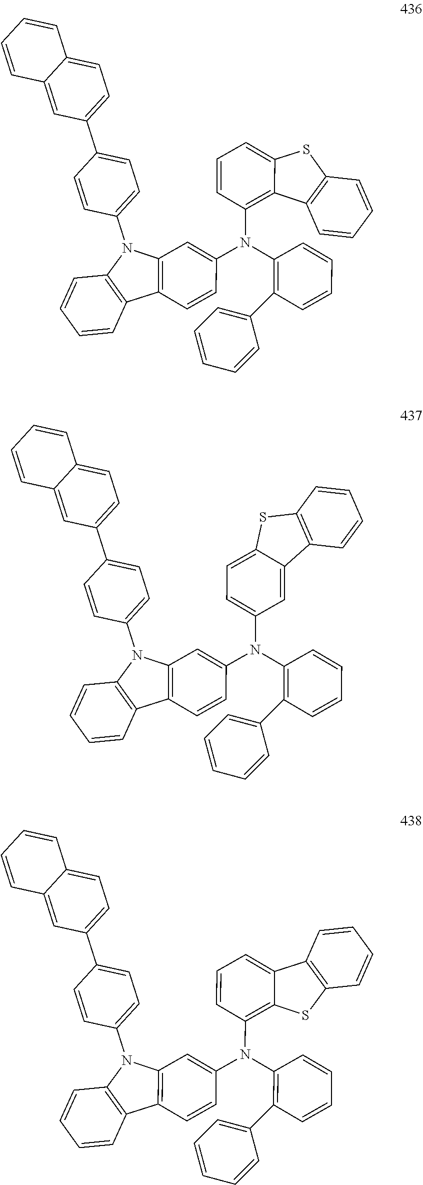 [SMILES: c1ccc(-c2ccccc2N(c2ccc3c4ccccc4n(-c4ccc(-c5ccc6ccccc6c5)cc4)c3c2)c2cccc3sc4ccccc4c23)cc1 ; c1ccc(-c2ccccc2N(c2ccc3sc4ccccc4c3c2)c2ccc3c4ccccc4n(-c4ccc(-c5ccc6ccccc6c5)cc4)c3c2)cc1 ; c1ccc(-c2ccccc2N(c2ccc3c4ccccc4n(-c4ccc(-c5ccc6ccccc6c5)cc4)c3c2)c2cccc3c2sc2ccccc23)cc1]

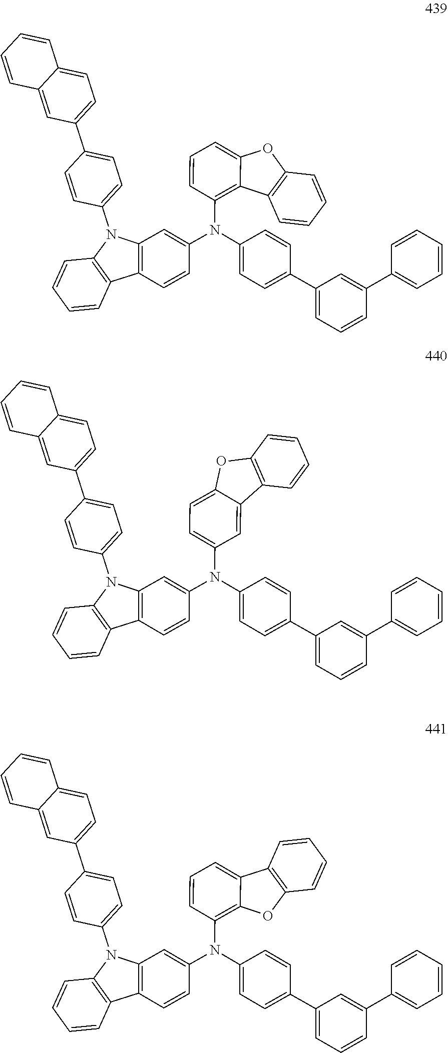 [SMILES: c1ccc(-c2cccc(-c3ccc(N(c4ccc5c6ccccc6n(-c6ccc(-c7ccc8ccccc8c7)cc6)c5c4)c4cccc5oc6ccccc6c45)cc3)c2)cc1 ; c1ccc(-c2cccc(-c3ccc(N(c4ccc5oc6ccccc6c5c4)c4ccc5c6ccccc6n(-c6ccc(-c7ccc8ccccc8c7)cc6)c5c4)cc3)c2)cc1 ; c1ccc(-c2cccc(-c3ccc(N(c4ccc5c6ccccc6n(-c6ccc(-c7ccc8ccccc8c7)cc6)c5c4)c4cccc5c4oc4ccccc45)cc3)c2)cc1]

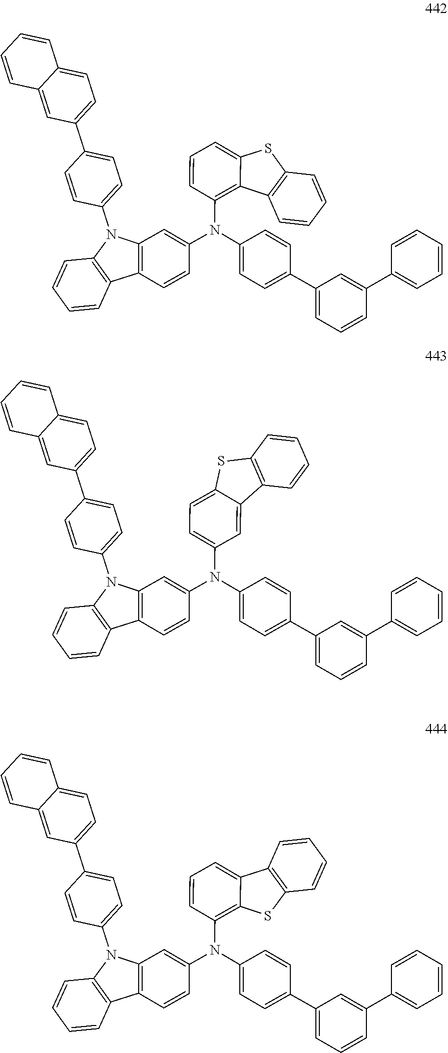 [SMILES: c1ccc(-c2cccc(-c3ccc(N(c4ccc5c6ccccc6n(-c6ccc(-c7ccc8ccccc8c7)cc6)c5c4)c4cccc5sc6ccccc6c45)cc3)c2)cc1 ; c1ccc(-c2cccc(-c3ccc(N(c4ccc5sc6ccccc6c5c4)c4ccc5c6ccccc6n(-c6ccc(-c7ccc8ccccc8c7)cc6)c5c4)cc3)c2)cc1 ; c1ccc(-c2cccc(-c3ccc(N(c4ccc5c6ccccc6n(-c6ccc(-c7ccc8ccccc8c7)cc6)c5c4)c4cccc5c4sc4ccccc45)cc3)c2)cc1]

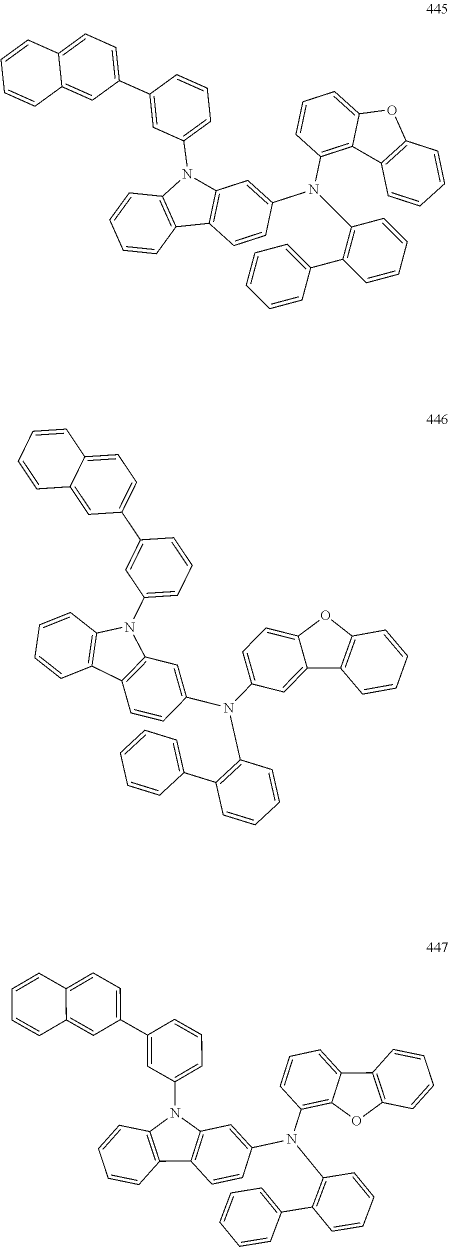 [SMILES: c1ccc(-c2ccccc2N(c2ccc3c4ccccc4n(-c4cccc(-c5ccc6ccccc6c5)c4)c3c2)c2cccc3oc4ccccc4c23)cc1 ; c1ccc(-c2ccccc2N(c2ccc3oc4ccccc4c3c2)c2ccc3c4ccccc4n(-c4cccc(-c5ccc6ccccc6c5)c4)c3c2)cc1 ; c1ccc(-c2ccccc2N(c2ccc3c4ccccc4n(-c4cccc(-c5ccc6ccccc6c5)c4)c3c2)c2cccc3c2oc2ccccc23)cc1]

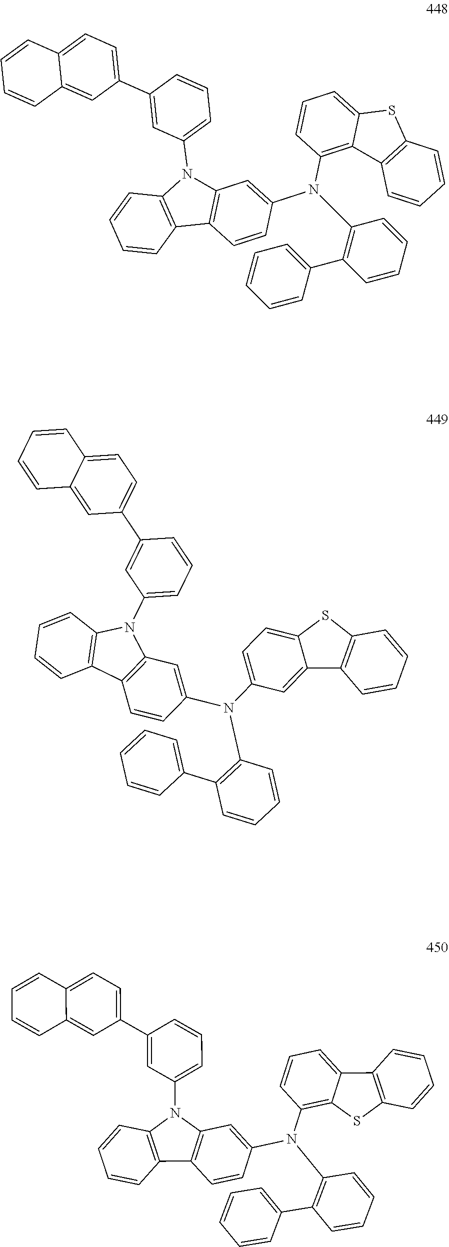 [SMILES: c1ccc(-c2ccccc2N(c2ccc3c4ccccc4n(-c4cccc(-c5ccc6ccccc6c5)c4)c3c2)c2cccc3sc4ccccc4c23)cc1 ; c1ccc(-c2ccccc2N(c2ccc3sc4ccccc4c3c2)c2ccc3c4ccccc4n(-c4cccc(-c5ccc6ccccc6c5)c4)c3c2)cc1 ; c1ccc(-c2ccccc2N(c2ccc3c4ccccc4n(-c4cccc(-c5ccc6ccccc6c5)c4)c3c2)c2cccc3c2sc2ccccc23)cc1]

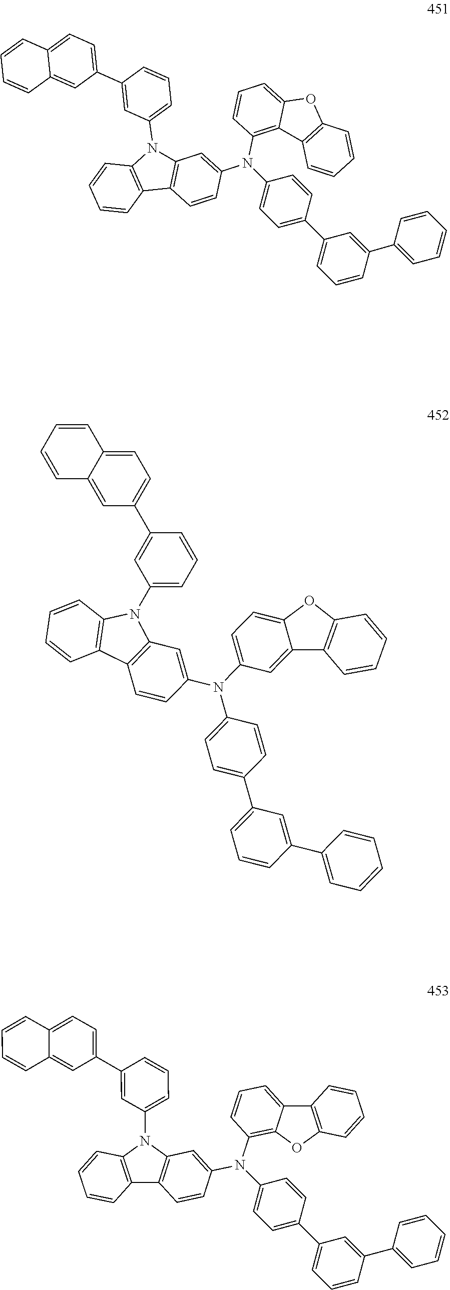 [SMILES: c1ccc(-c2cccc(-c3ccc(N(c4ccc5c6ccccc6n(-c6cccc(-c7ccc8ccccc8c7)c6)c5c4)c4cccc5oc6ccccc6c45)cc3)c2)cc1 ; c1ccc(-c2cccc(-c3ccc(N(c4ccc5oc6ccccc6c5c4)c4ccc5c6ccccc6n(-c6cccc(-c7ccc8ccccc8c7)c6)c5c4)cc3)c2)cc1 ; c1ccc(-c2cccc(-c3ccc(N(c4ccc5c6ccccc6n(-c6cccc(-c7ccc8ccccc8c7)c6)c5c4)c4cccc5c4oc4ccccc45)cc3)c2)cc1]

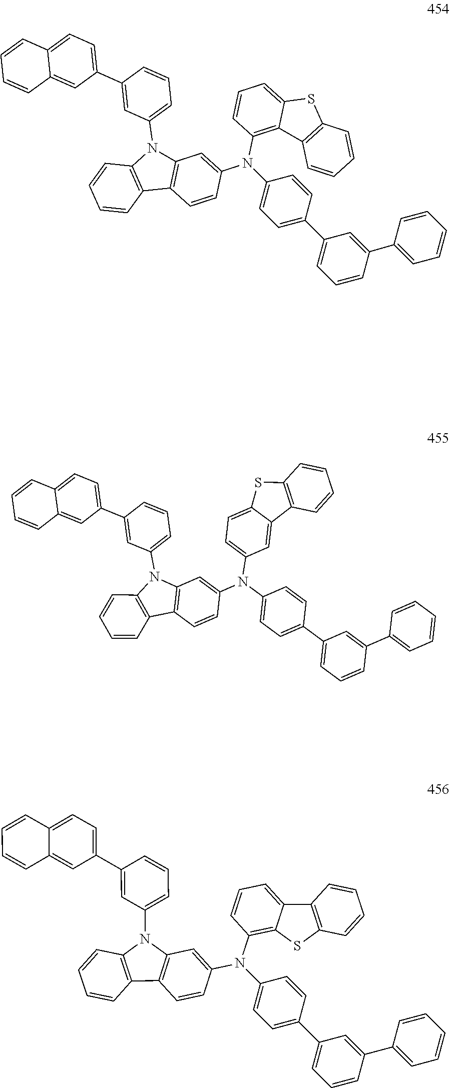 [SMILES: c1ccc(-c2cccc(-c3ccc(N(c4ccc5c6ccccc6n(-c6cccc(-c7ccc8ccccc8c7)c6)c5c4)c4cccc5sc6ccccc6c45)cc3)c2)cc1 ; c1ccc(-c2cccc(-c3ccc(N(c4ccc5sc6ccccc6c5c4)c4ccc5c6ccccc6n(-c6cccc(-c7ccc8ccccc8c7)c6)c5c4)cc3)c2)cc1 ; c1ccc(-c2cccc(-c3ccc(N(c4ccc5c6ccccc6n(-c6cccc(-c7ccc8ccccc8c7)c6)c5c4)c4cccc5c4sc4ccccc45)cc3)c2)cc1]

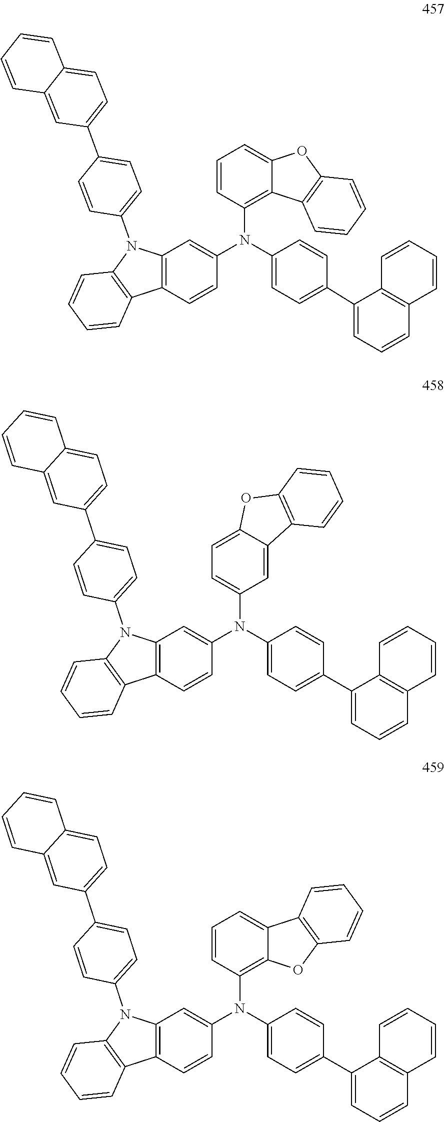 [SMILES: c1ccc2cc(-c3ccc(-n4c5ccccc5c5ccc(N(c6ccc(-c7cccc8ccccc78)cc6)c6cccc7oc8ccccc8c67)cc54)cc3)ccc2c1 ; c1ccc2cc(-c3ccc(-n4c5ccccc5c5ccc(N(c6ccc(-c7cccc8ccccc78)cc6)c6ccc7oc8ccccc8c7c6)cc54)cc3)ccc2c1 ; c1ccc2cc(-c3ccc(-n4c5ccccc5c5ccc(N(c6ccc(-c7cccc8ccccc78)cc6)c6cccc7c6oc6ccccc67)cc54)cc3)ccc2c1]

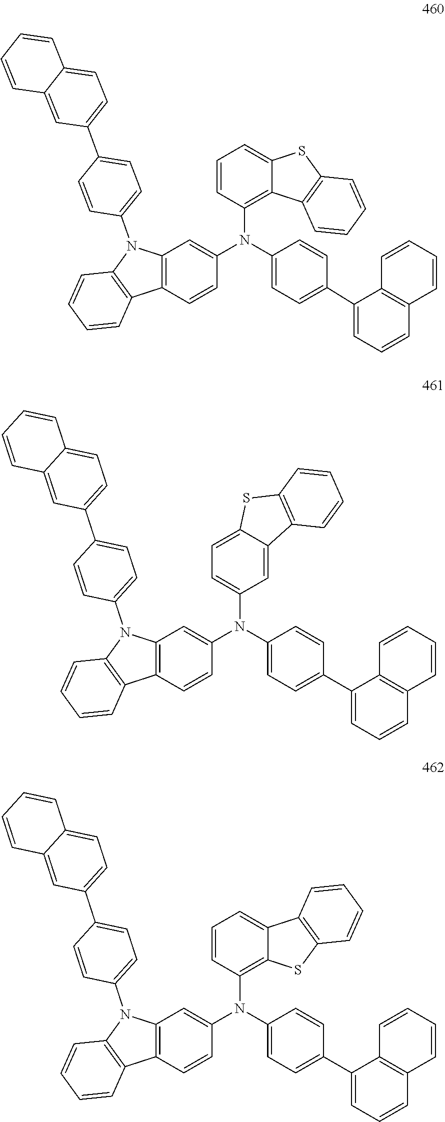 [SMILES: c1ccc2cc(-c3ccc(-n4c5ccccc5c5ccc(N(c6ccc(-c7cccc8ccccc78)cc6)c6cccc7sc8ccccc8c67)cc54)cc3)ccc2c1 ; c1ccc2cc(-c3ccc(-n4c5ccccc5c5ccc(N(c6ccc(-c7cccc8ccccc78)cc6)c6ccc7sc8ccccc8c7c6)cc54)cc3)ccc2c1 ; c1ccc2cc(-c3ccc(-n4c5ccccc5c5ccc(N(c6ccc(-c7cccc8ccccc78)cc6)c6cccc7c6sc6ccccc67)cc54)cc3)ccc2c1]

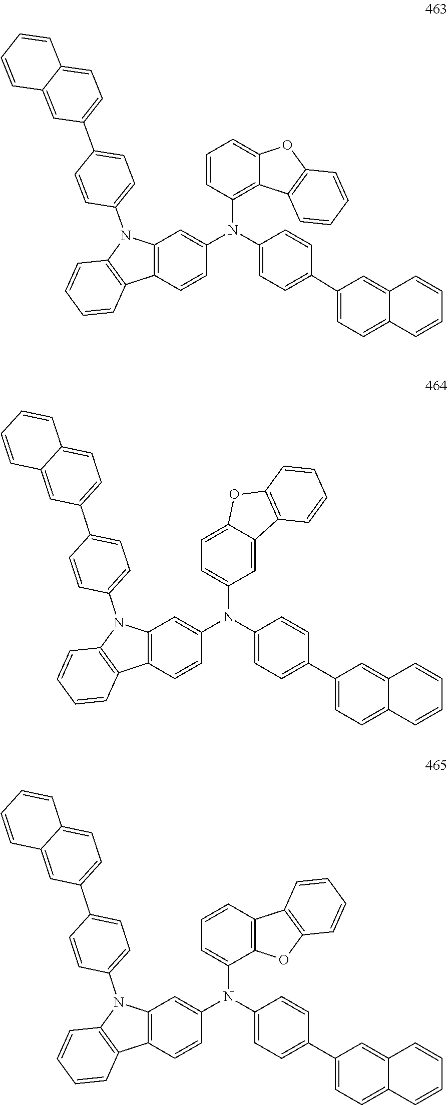 [SMILES: c1ccc2cc(-c3ccc(N(c4ccc5c6ccccc6n(-c6ccc(-c7ccc8ccccc8c7)cc6)c5c4)c4cccc5oc6ccccc6c45)cc3)ccc2c1 ; c1ccc2cc(-c3ccc(N(c4ccc5oc6ccccc6c5c4)c4ccc5c6ccccc6n(-c6ccc(-c7ccc8ccccc8c7)cc6)c5c4)cc3)ccc2c1 ; c1ccc2cc(-c3ccc(N(c4ccc5c6ccccc6n(-c6ccc(-c7ccc8ccccc8c7)cc6)c5c4)c4cccc5c4oc4ccccc45)cc3)ccc2c1]

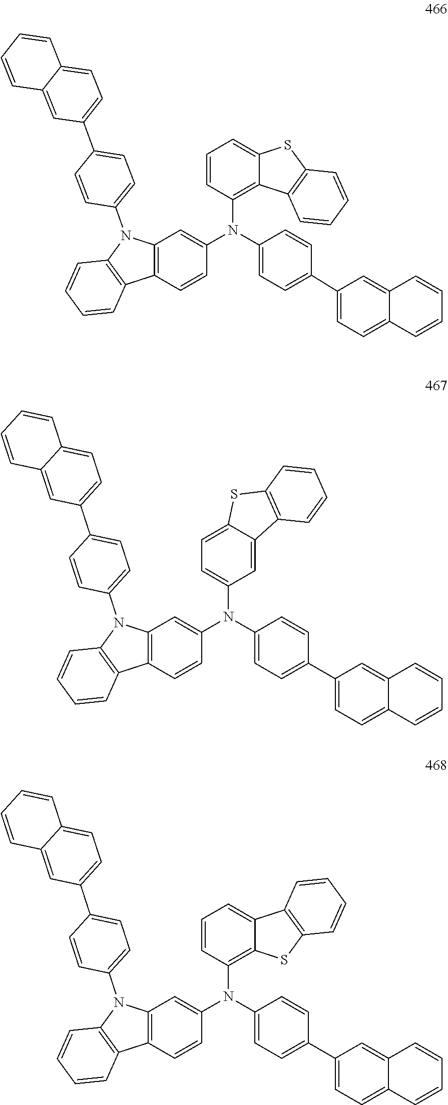 [SMILES: c1ccc2cc(-c3ccc(N(c4ccc5c6ccccc6n(-c6ccc(-c7ccc8ccccc8c7)cc6)c5c4)c4cccc5sc6ccccc6c45)cc3)ccc2c1 ; c1ccc2cc(-c3ccc(N(c4ccc5sc6ccccc6c5c4)c4ccc5c6ccccc6n(-c6ccc(-c7ccc8ccccc8c7)cc6)c5c4)cc3)ccc2c1 ; c1ccc2cc(-c3ccc(N(c4ccc5c6ccccc6n(-c6ccc(-c7ccc8ccccc8c7)cc6)c5c4)c4cccc5c4sc4ccccc45)cc3)ccc2c1]

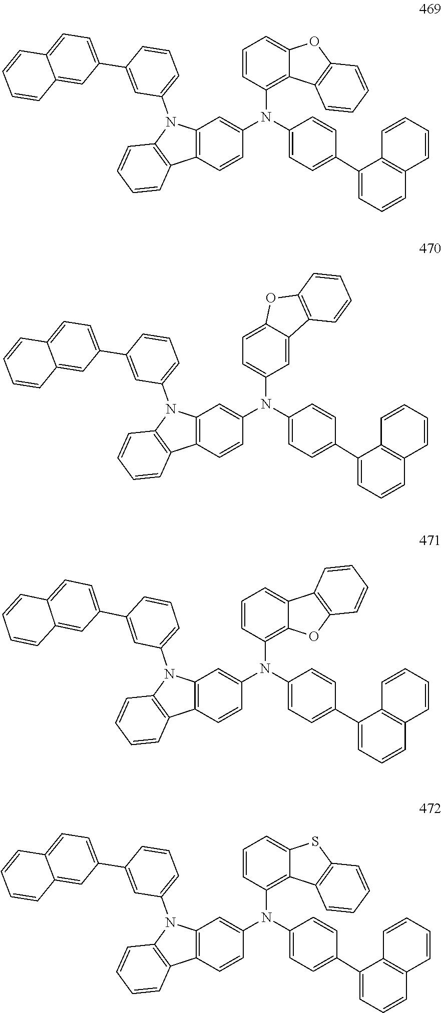 [SMILES: c1cc(-c2ccc3ccccc3c2)cc(-n2c3ccccc3c3ccc(N(c4ccc(-c5cccc6ccccc56)cc4)c4cccc5oc6ccccc6c45)cc32)c1 ; c1cc(-c2ccc3ccccc3c2)cc(-n2c3ccccc3c3ccc(N(c4ccc(-c5cccc6ccccc56)cc4)c4ccc5oc6ccccc6c5c4)cc32)c1 ; c1cc(-c2ccc3ccccc3c2)cc(-n2c3ccccc3c3ccc(N(c4ccc(-c5cccc6ccccc56)cc4)c4cccc5c4oc4ccccc45)cc32)c1 ; c1cc(-c2ccc3ccccc3c2)cc(-n2c3ccccc3c3ccc(N(c4ccc(-c5cccc6ccccc56)cc4)c4cccc5sc6ccccc6c45)cc32)c1]

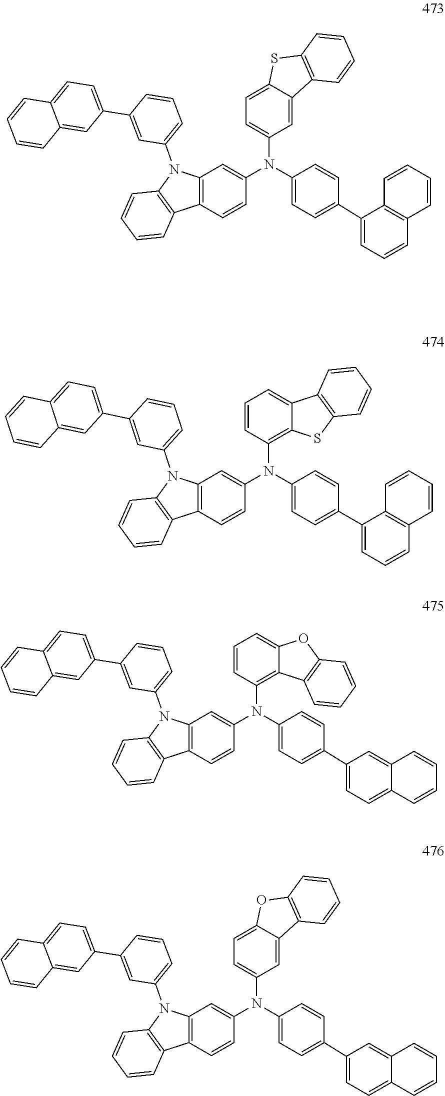 [SMILES: c1cc(-c2ccc3ccccc3c2)cc(-n2c3ccccc3c3ccc(N(c4ccc(-c5cccc6ccccc56)cc4)c4ccc5sc6ccccc6c5c4)cc32)c1 ; c1cc(-c2ccc3ccccc3c2)cc(-n2c3ccccc3c3ccc(N(c4ccc(-c5cccc6ccccc56)cc4)c4cccc5c4sc4ccccc45)cc32)c1 ; c1cc(-c2ccc3ccccc3c2)cc(-n2c3ccccc3c3ccc(N(c4ccc(-c5ccc6ccccc6c5)cc4)c4cccc5oc6ccccc6c45)cc32)c1 ; c1cc(-c2ccc3ccccc3c2)cc(-n2c3ccccc3c3ccc(N(c4ccc(-c5ccc6ccccc6c5)cc4)c4ccc5oc6ccccc6c5c4)cc32)c1]

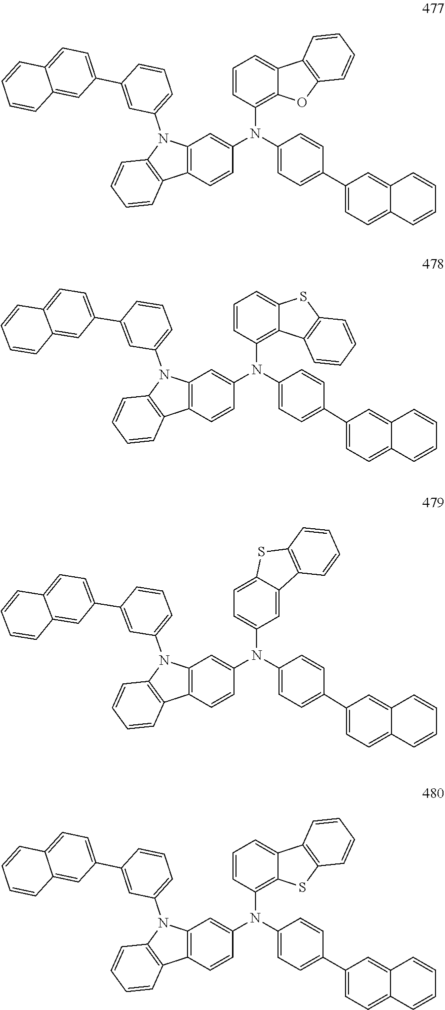 [SMILES: c1cc(-c2ccc3ccccc3c2)cc(-n2c3ccccc3c3ccc(N(c4ccc(-c5ccc6ccccc6c5)cc4)c4cccc5c4oc4ccccc45)cc32)c1 ; c1cc(-c2ccc3ccccc3c2)cc(-n2c3ccccc3c3ccc(N(c4ccc(-c5ccc6ccccc6c5)cc4)c4cccc5sc6ccccc6c45)cc32)c1 ; c1cc(-c2ccc3ccccc3c2)cc(-n2c3ccccc3c3ccc(N(c4ccc(-c5ccc6ccccc6c5)cc4)c4ccc5sc6ccccc6c5c4)cc32)c1 ; c1cc(-c2ccc3ccccc3c2)cc(-n2c3ccccc3c3ccc(N(c4ccc(-c5ccc6ccccc6c5)cc4)c4cccc5c4sc4ccccc45)cc32)c1]

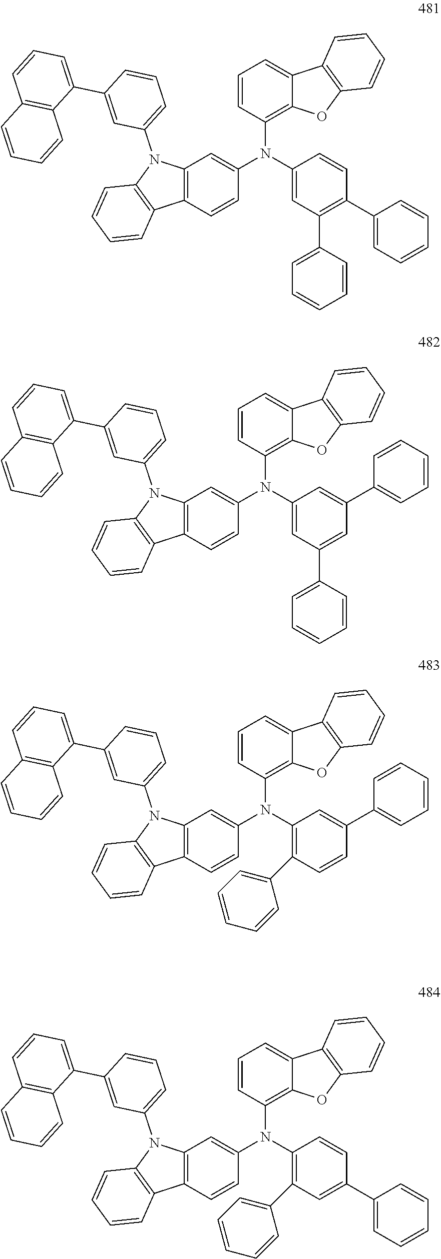 [SMILES: c1ccc(-c2ccc(N(c3ccc4c5ccccc5n(-c5cccc(-c6cccc7ccccc67)c5)c4c3)c3cccc4c3oc3ccccc34)cc2-c2ccccc2)cc1 ; c1ccc(-c2cc(-c3ccccc3)cc(N(c3ccc4c5ccccc5n(-c5cccc(-c6cccc7ccccc67)c5)c4c3)c3cccc4c3oc3ccccc34)c2)cc1 ; c1ccc(-c2ccc(-c3ccccc3)c(N(c3ccc4c5ccccc5n(-c5cccc(-c6cccc7ccccc67)c5)c4c3)c3cccc4c3oc3ccccc34)c2)cc1 ; c1ccc(-c2ccc(N(c3ccc4c5ccccc5n(-c5cccc(-c6cccc7ccccc67)c5)c4c3)c3cccc4c3oc3ccccc34)c(-c3ccccc3)c2)cc1]

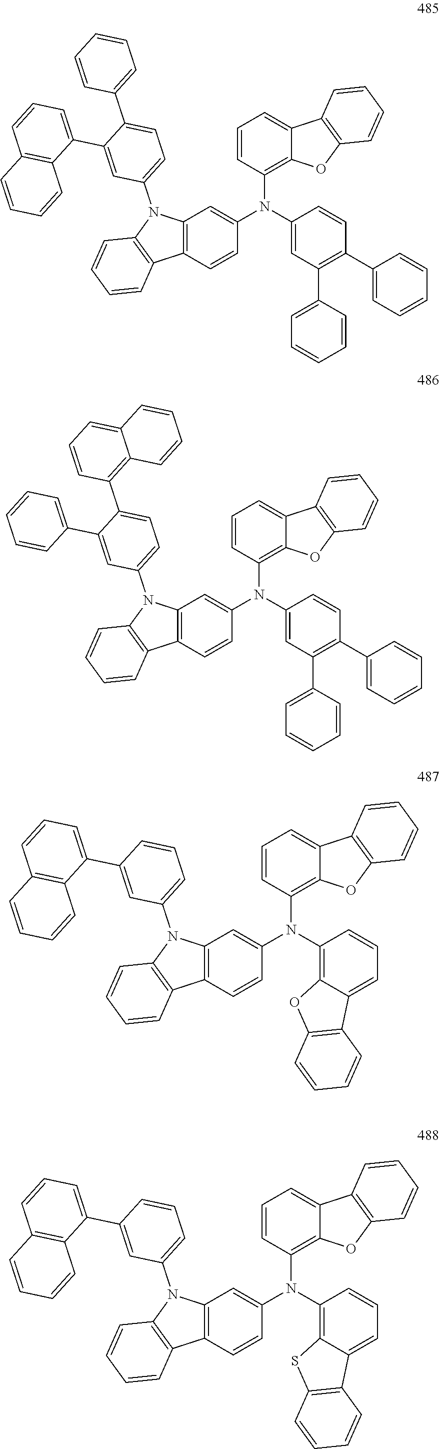 [SMILES: c1ccc(-c2ccc(N(c3ccc4c5ccccc5n(-c5ccc(-c6ccccc6)c(-c6cccc7ccccc67)c5)c4c3)c3cccc4c3oc3ccccc34)cc2-c2ccccc2)cc1 ; c1ccc(-c2ccc(N(c3ccc4c5ccccc5n(-c5ccc(-c6cccc7ccccc67)c(-c6ccccc6)c5)c4c3)c3cccc4c3oc3ccccc34)cc2-c2ccccc2)cc1 ; c1cc(-c2cccc3ccccc23)cc(-n2c3ccccc3c3ccc(N(c4cccc5c4oc4ccccc45)c4cccc5c4oc4ccccc45)cc32)c1 ; c1cc(-c2cccc3ccccc23)cc(-n2c3ccccc3c3ccc(N(c4cccc5c4oc4ccccc45)c4cccc5c4sc4ccccc45)cc32)c1]

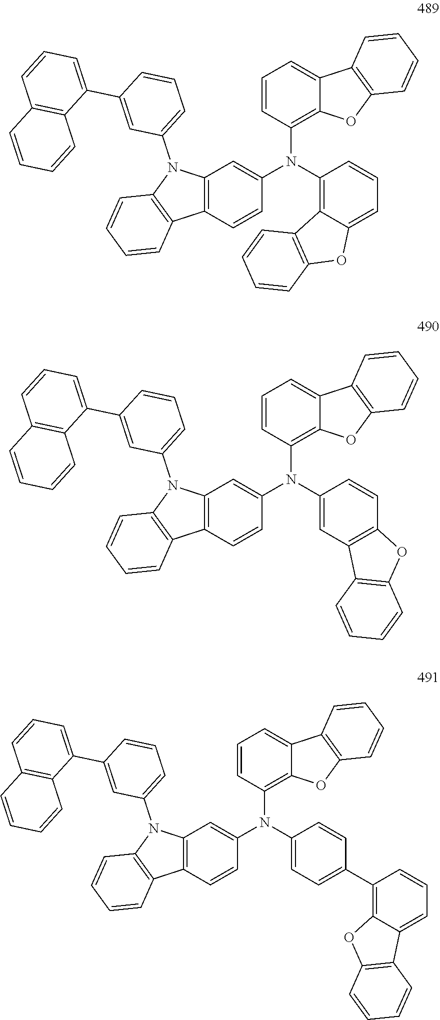 [SMILES: c1cc(-c2cccc3ccccc23)cc(-n2c3ccccc3c3ccc(N(c4cccc5c4oc4ccccc45)c4cccc5oc6ccccc6c45)cc32)c1 ; c1cc(-c2cccc3ccccc23)cc(-n2c3ccccc3c3ccc(N(c4ccc5oc6ccccc6c5c4)c4cccc5c4oc4ccccc45)cc32)c1 ; c1cc(-c2cccc3ccccc23)cc(-n2c3ccccc3c3ccc(N(c4ccc(-c5cccc6c5oc5ccccc56)cc4)c4cccc5c4oc4ccccc45)cc32)c1]

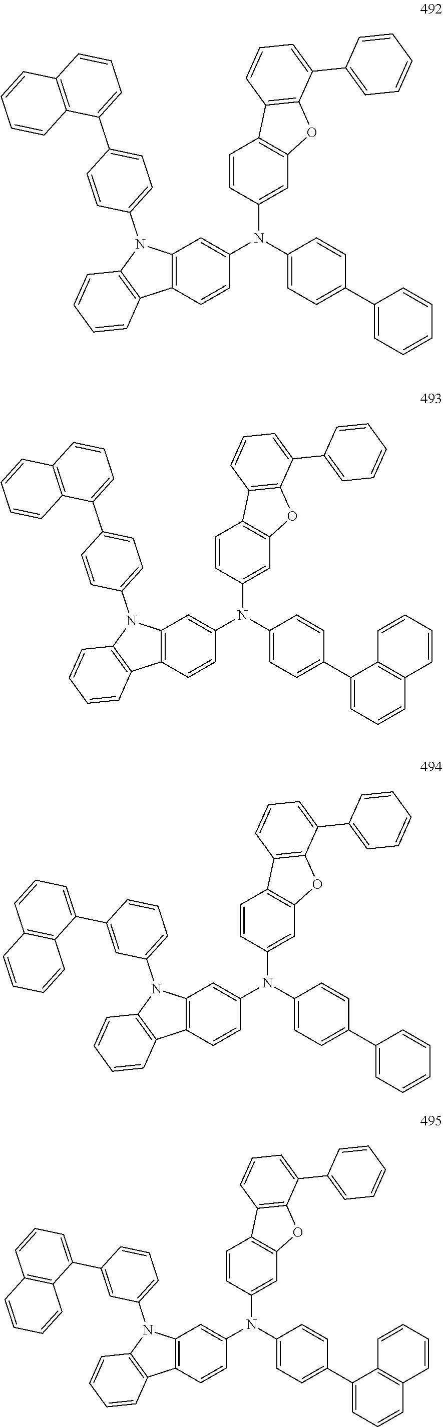 [SMILES: c1ccc(-c2ccc(N(c3ccc4c(c3)oc3c(-c5ccccc5)cccc34)c3ccc4c5ccccc5n(-c5ccc(-c6cccc7ccccc67)cc5)c4c3)cc2)cc1 ; c1ccc(-c2cccc3c2oc2cc(N(c4ccc(-c5cccc6ccccc56)cc4)c4ccc5c6ccccc6n(-c6ccc(-c7cccc8ccccc78)cc6)c5c4)ccc23)cc1 ; c1ccc(-c2ccc(N(c3ccc4c(c3)oc3c(-c5ccccc5)cccc34)c3ccc4c5ccccc5n(-c5cccc(-c6cccc7ccccc67)c5)c4c3)cc2)cc1 ; c1ccc(-c2cccc3c2oc2cc(N(c4ccc(-c5cccc6ccccc56)cc4)c4ccc5c6ccccc6n(-c6cccc(-c7cccc8ccccc78)c6)c5c4)ccc23)cc1]

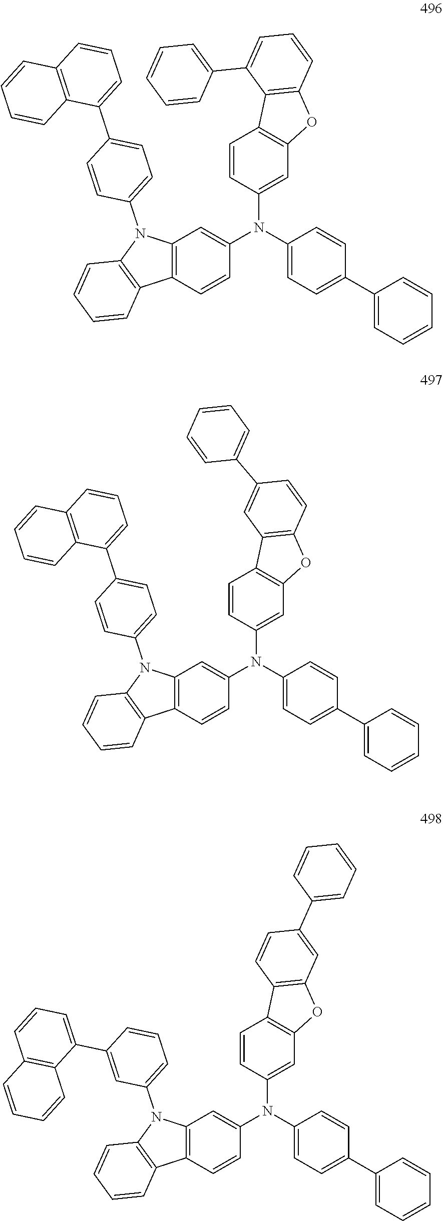 [SMILES: c1ccc(-c2ccc(N(c3ccc4c(c3)oc3cccc(-c5ccccc5)c34)c3ccc4c5ccccc5n(-c5ccc(-c6cccc7ccccc67)cc5)c4c3)cc2)cc1 ; c1ccc(-c2ccc(N(c3ccc4c(c3)oc3ccc(-c5ccccc5)cc34)c3ccc4c5ccccc5n(-c5ccc(-c6cccc7ccccc67)cc5)c4c3)cc2)cc1 ; c1ccc(-c2ccc(N(c3ccc4c(c3)oc3cc(-c5ccccc5)ccc34)c3ccc4c5ccccc5n(-c5cccc(-c6cccc7ccccc67)c5)c4c3)cc2)cc1]

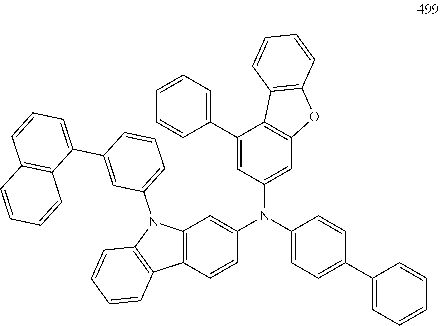 [SMILES: c1ccc(-c2ccc(N(c3cc(-c4ccccc4)c4c(c3)oc3ccccc34)c3ccc4c5ccccc5n(-c5cccc(-c6cccc7ccccc67)c5)c4c3)cc2)cc1]

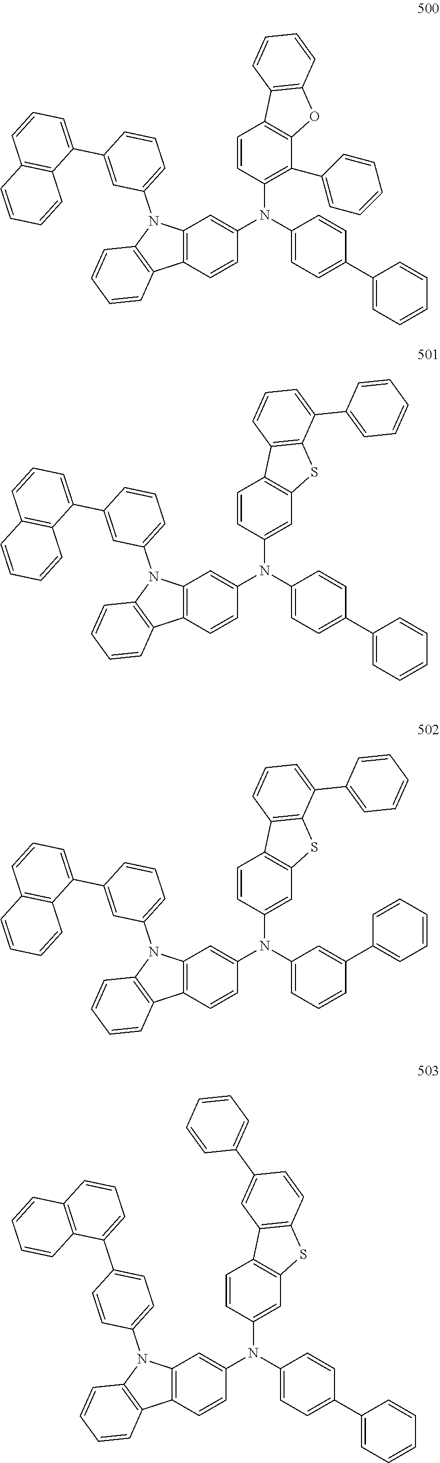 [SMILES: c1ccc(-c2ccc(N(c3ccc4c5ccccc5n(-c5cccc(-c6cccc7ccccc67)c5)c4c3)c3ccc4c(oc5ccccc54)c3-c3ccccc3)cc2)cc1 ; c1ccc(-c2ccc(N(c3ccc4c(c3)sc3c(-c5ccccc5)cccc34)c3ccc4c5ccccc5n(-c5cccc(-c6cccc7ccccc67)c5)c4c3)cc2)cc1 ; c1ccc(-c2cccc(N(c3ccc4c(c3)sc3c(-c5ccccc5)cccc34)c3ccc4c5ccccc5n(-c5cccc(-c6cccc7ccccc67)c5)c4c3)c2)cc1 ; c1ccc(-c2ccc(N(c3ccc4c(c3)sc3ccc(-c5ccccc5)cc34)c3ccc4c5ccccc5n(-c5ccc(-c6cccc7ccccc67)cc5)c4c3)cc2)cc1]

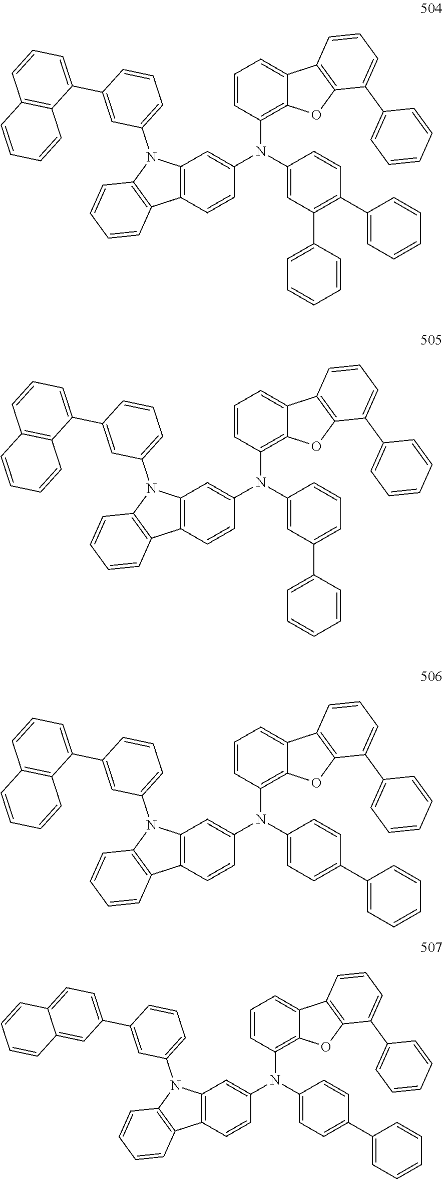 [SMILES: c1ccc(-c2ccc(N(c3ccc4c5ccccc5n(-c5cccc(-c6cccc7ccccc67)c5)c4c3)c3cccc4c3oc3c(-c5ccccc5)cccc34)cc2-c2ccccc2)cc1 ; c1ccc(-c2cccc(N(c3ccc4c5ccccc5n(-c5cccc(-c6cccc7ccccc67)c5)c4c3)c3cccc4c3oc3c(-c5ccccc5)cccc34)c2)cc1 ; c1ccc(-c2ccc(N(c3ccc4c5ccccc5n(-c5cccc(-c6cccc7ccccc67)c5)c4c3)c3cccc4c3oc3c(-c5ccccc5)cccc34)cc2)cc1 ; c1ccc(-c2ccc(N(c3ccc4c5ccccc5n(-c5cccc(-c6ccc7ccccc7c6)c5)c4c3)c3cccc4c3oc3c(-c5ccccc5)cccc34)cc2)cc1]

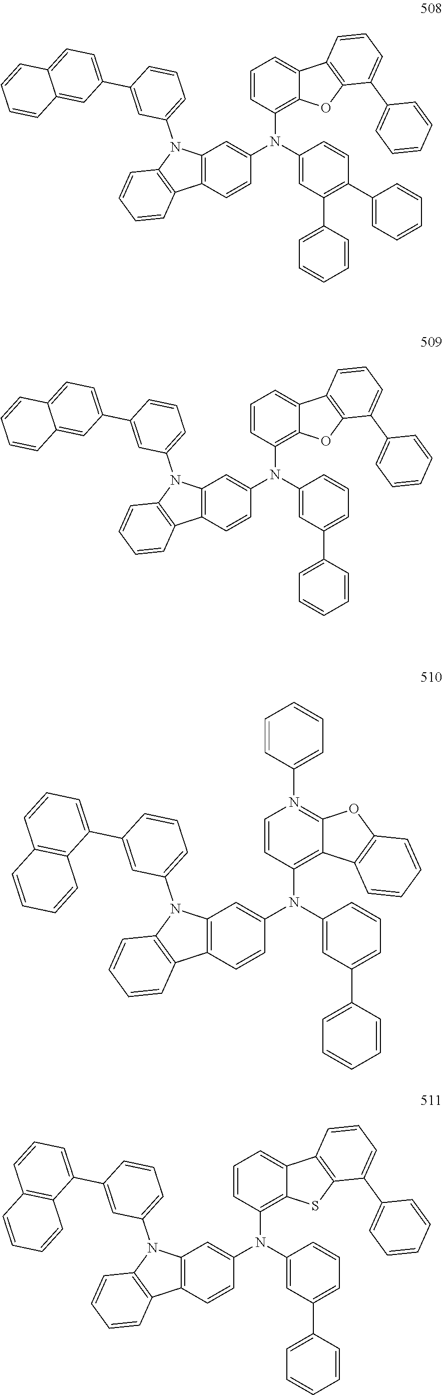

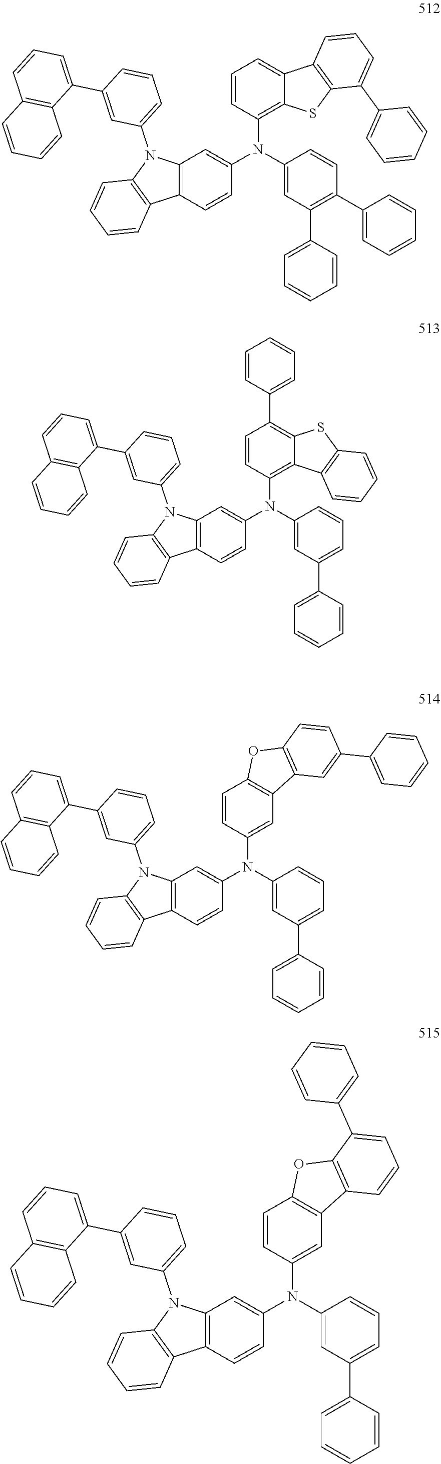

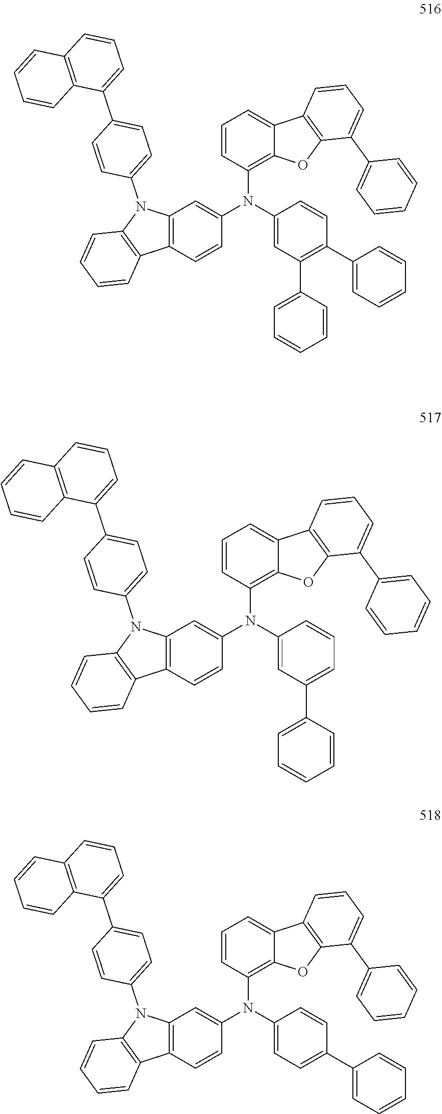 [SMILES: c1ccc(-c2ccc(N(c3ccc4c5ccccc5n(-c5ccc(-c6cccc7ccccc67)cc5)c4c3)c3cccc4c3oc3c(-c5ccccc5)cccc34)cc2-c2ccccc2)cc1 ; c1ccc(-c2cccc(N(c3ccc4c5ccccc5n(-c5ccc(-c6cccc7ccccc67)cc5)c4c3)c3cccc4c3oc3c(-c5ccccc5)cccc34)c2)cc1 ; c1ccc(-c2ccc(N(c3ccc4c5ccccc5n(-c5ccc(-c6cccc7ccccc67)cc5)c4c3)c3cccc4c3oc3c(-c5ccccc5)cccc34)cc2)cc1]

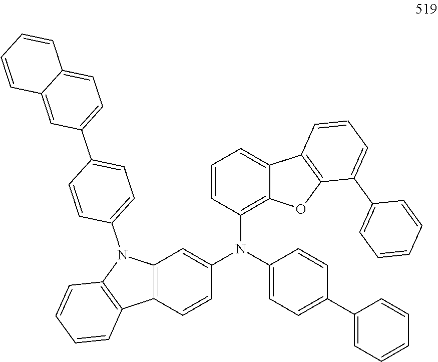 [SMILES: c1ccc(-c2ccc(N(c3ccc4c5ccccc5n(-c5ccc(-c6ccc7ccccc7c6)cc5)c4c3)c3cccc4c3oc3c(-c5ccccc5)cccc34)cc2)cc1]

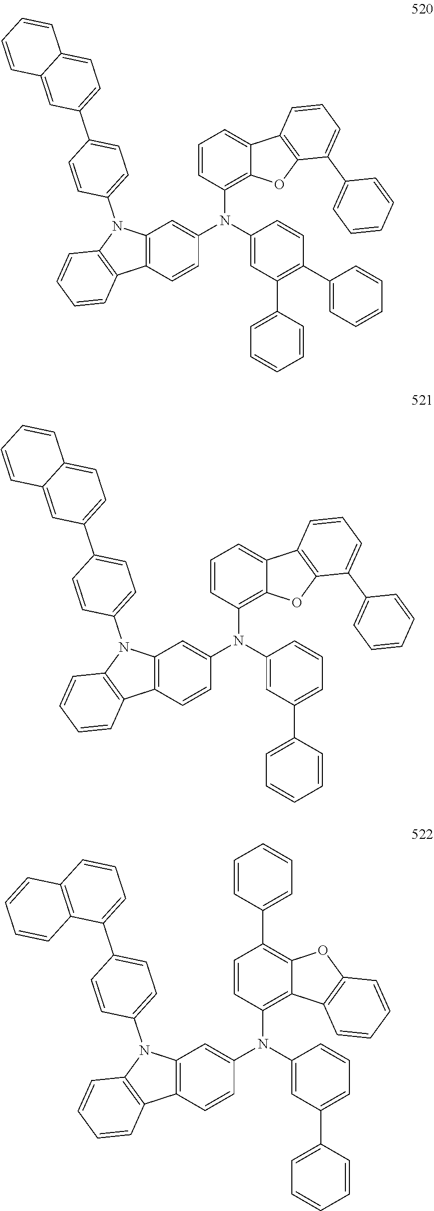 [SMILES: c1ccc(-c2ccc(N(c3ccc4c5ccccc5n(-c5ccc(-c6ccc7ccccc7c6)cc5)c4c3)c3cccc4c3oc3c(-c5ccccc5)cccc34)cc2-c2ccccc2)cc1 ; c1ccc(-c2cccc(N(c3ccc4c5ccccc5n(-c5ccc(-c6ccc7ccccc7c6)cc5)c4c3)c3cccc4c3oc3c(-c5ccccc5)cccc34)c2)cc1 ; c1ccc(-c2cccc(N(c3ccc4c5ccccc5n(-c5ccc(-c6cccc7ccccc67)cc5)c4c3)c3ccc(-c4ccccc4)c4oc5ccccc5c34)c2)cc1]

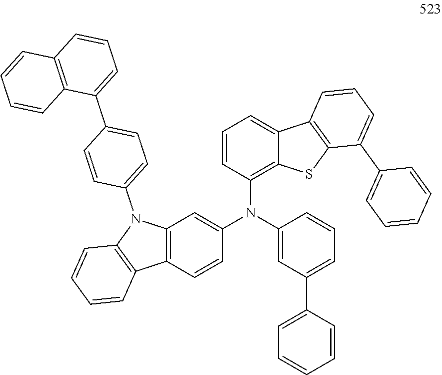 [SMILES: c1ccc(-c2cccc(N(c3ccc4c5ccccc5n(-c5ccc(-c6cccc7ccccc67)cc5)c4c3)c3cccc4c3sc3c(-c5ccccc5)cccc34)c2)cc1]

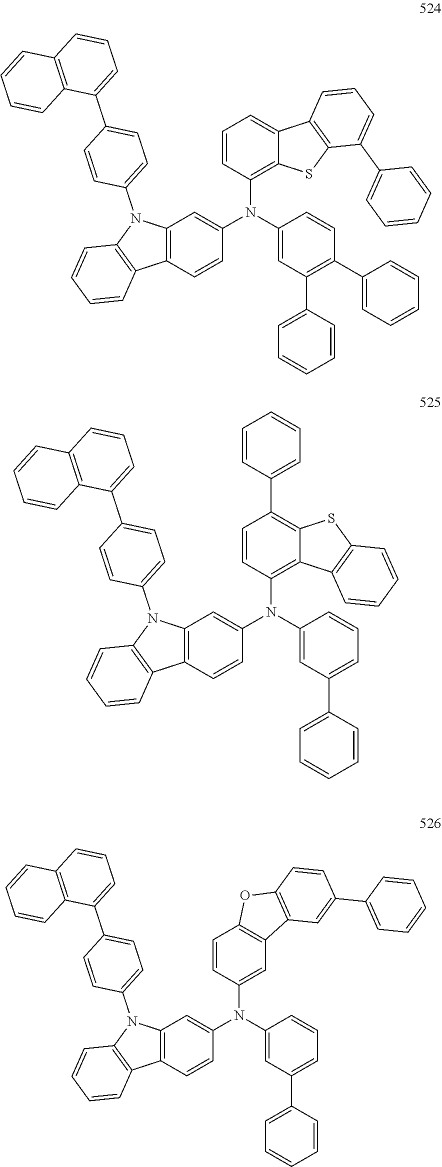 [SMILES: c1ccc(-c2ccc(N(c3ccc4c5ccccc5n(-c5ccc(-c6cccc7ccccc67)cc5)c4c3)c3cccc4c3sc3c(-c5ccccc5)cccc34)cc2-c2ccccc2)cc1 ; c1ccc(-c2cccc(N(c3ccc4c5ccccc5n(-c5ccc(-c6cccc7ccccc67)cc5)c4c3)c3ccc(-c4ccccc4)c4sc5ccccc5c34)c2)cc1 ; c1ccc(-c2cccc(N(c3ccc4oc5ccc(-c6ccccc6)cc5c4c3)c3ccc4c5ccccc5n(-c5ccc(-c6cccc7ccccc67)cc5)c4c3)c2)cc1]

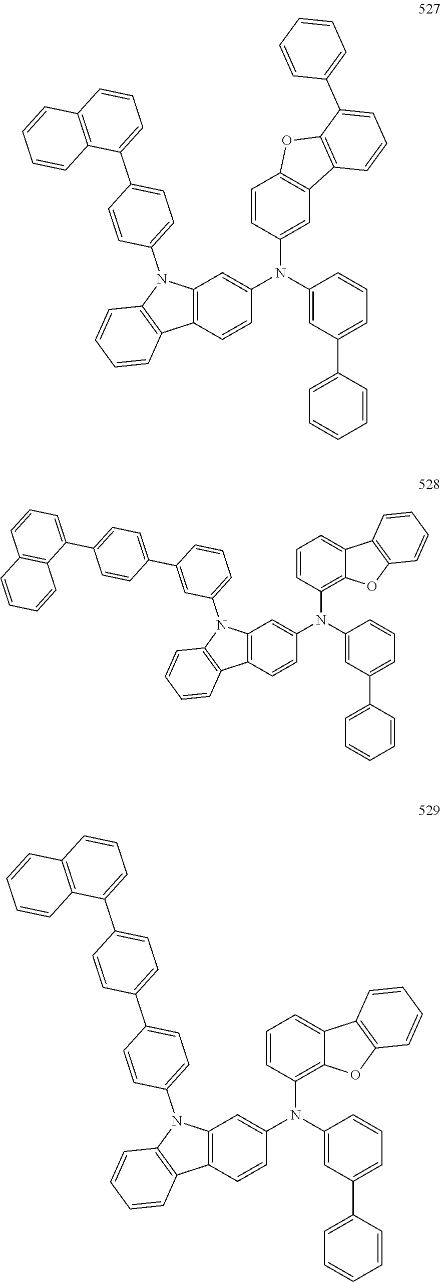 [SMILES: c1ccc(-c2cccc(N(c3ccc4oc5c(-c6ccccc6)cccc5c4c3)c3ccc4c5ccccc5n(-c5ccc(-c6cccc7ccccc67)cc5)c4c3)c2)cc1 ; c1ccc(-c2cccc(N(c3ccc4c5ccccc5n(-c5cccc(-c6ccc(-c7cccc8ccccc78)cc6)c5)c4c3)c3cccc4c3oc3ccccc34)c2)cc1 ; c1ccc(-c2cccc(N(c3ccc4c5ccccc5n(-c5ccc(-c6ccc(-c7cccc8ccccc78)cc6)cc5)c4c3)c3cccc4c3oc3ccccc34)c2)cc1]

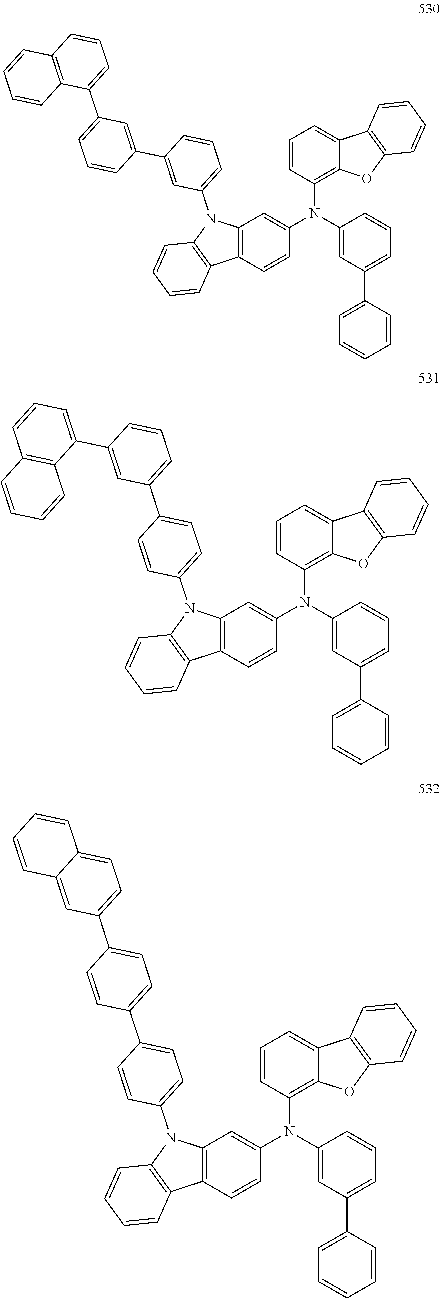 [SMILES: c1ccc(-c2cccc(N(c3ccc4c5ccccc5n(-c5cccc(-c6cccc(-c7cccc8ccccc78)c6)c5)c4c3)c3cccc4c3oc3ccccc34)c2)cc1 ; c1ccc(-c2cccc(N(c3ccc4c5ccccc5n(-c5ccc(-c6cccc(-c7cccc8ccccc78)c6)cc5)c4c3)c3cccc4c3oc3ccccc34)c2)cc1 ; c1ccc(-c2cccc(N(c3ccc4c5ccccc5n(-c5ccc(-c6ccc(-c7ccc8ccccc8c7)cc6)cc5)c4c3)c3cccc4c3oc3ccccc34)c2)cc1]

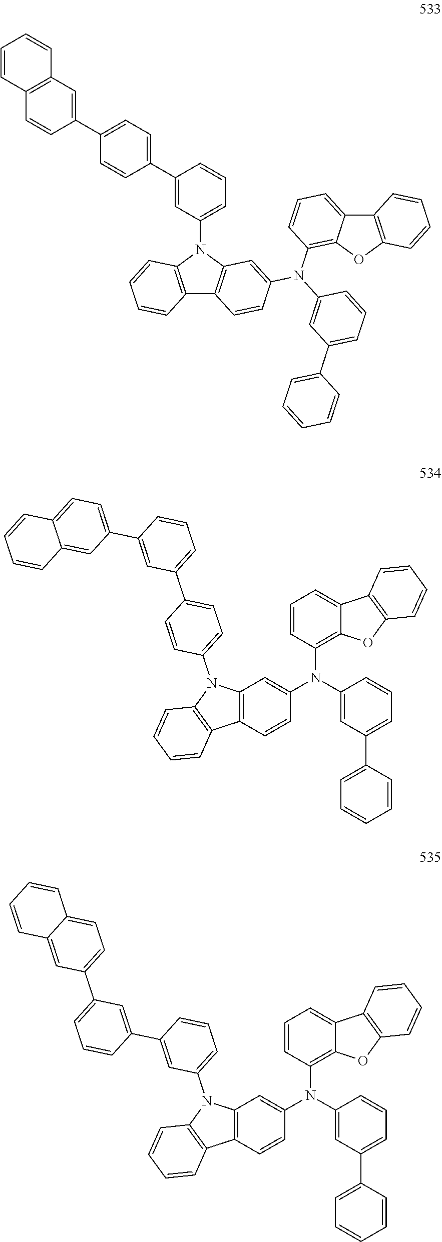 [SMILES: c1ccc(-c2cccc(N(c3ccc4c5ccccc5n(-c5cccc(-c6ccc(-c7ccc8ccccc8c7)cc6)c5)c4c3)c3cccc4c3oc3ccccc34)c2)cc1 ; c1ccc(-c2cccc(N(c3ccc4c5ccccc5n(-c5ccc(-c6cccc(-c7ccc8ccccc8c7)c6)cc5)c4c3)c3cccc4c3oc3ccccc34)c2)cc1 ; c1ccc(-c2cccc(N(c3ccc4c5ccccc5n(-c5cccc(-c6cccc(-c7ccc8ccccc8c7)c6)c5)c4c3)c3cccc4c3oc3ccccc34)c2)cc1]

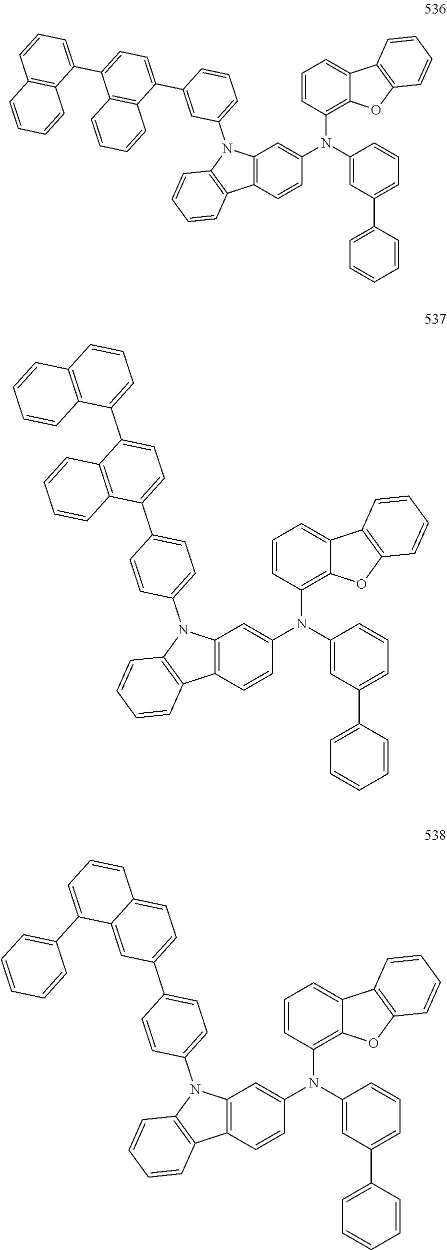 [SMILES: c1ccc(-c2cccc(N(c3ccc4c5ccccc5n(-c5cccc(-c6ccc(-c7cccc8ccccc78)c7ccccc67)c5)c4c3)c3cccc4c3oc3ccccc34)c2)cc1 ; c1ccc(-c2cccc(N(c3ccc4c5ccccc5n(-c5ccc(-c6ccc(-c7cccc8ccccc78)c7ccccc67)cc5)c4c3)c3cccc4c3oc3ccccc34)c2)cc1 ; c1ccc(-c2cccc(N(c3ccc4c5ccccc5n(-c5ccc(-c6ccc7cccc(-c8ccccc8)c7c6)cc5)c4c3)c3cccc4c3oc3ccccc34)c2)cc1]

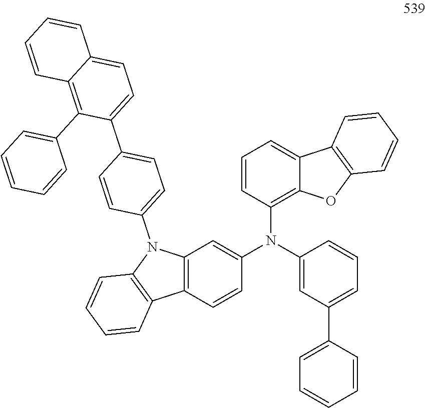 [SMILES: c1ccc(-c2cccc(N(c3ccc4c5ccccc5n(-c5ccc(-c6ccc7ccccc7c6-c6ccccc6)cc5)c4c3)c3cccc4c3oc3ccccc34)c2)cc1]

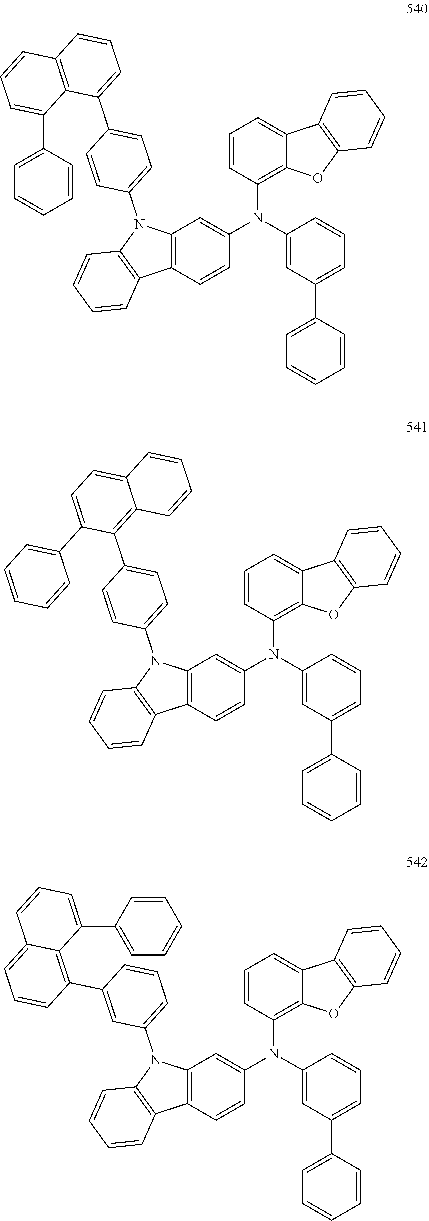 [SMILES: c1ccc(-c2cccc(N(c3ccc4c5ccccc5n(-c5ccc(-c6cccc7cccc(-c8ccccc8)c67)cc5)c4c3)c3cccc4c3oc3ccccc34)c2)cc1 ; c1ccc(-c2cccc(N(c3ccc4c5ccccc5n(-c5ccc(-c6c(-c7ccccc7)ccc7ccccc67)cc5)c4c3)c3cccc4c3oc3ccccc34)c2)cc1 ; c1ccc(-c2cccc(N(c3ccc4c5ccccc5n(-c5cccc(-c6cccc7cccc(-c8ccccc8)c67)c5)c4c3)c3cccc4c3oc3ccccc34)c2)cc1]

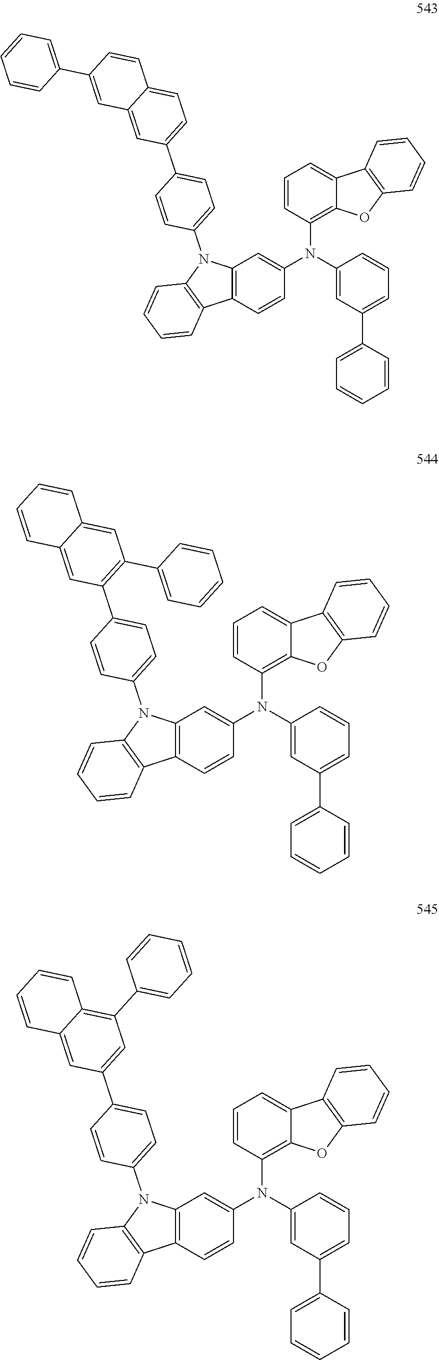 [SMILES: c1ccc(-c2cccc(N(c3ccc4c5ccccc5n(-c5ccc(-c6ccc7ccc(-c8ccccc8)cc7c6)cc5)c4c3)c3cccc4c3oc3ccccc34)c2)cc1 ; c1ccc(-c2cccc(N(c3ccc4c5ccccc5n(-c5ccc(-c6cc7ccccc7cc6-c6ccccc6)cc5)c4c3)c3cccc4c3oc3ccccc34)c2)cc1 ; c1ccc(-c2cccc(N(c3ccc4c5ccccc5n(-c5ccc(-c6cc(-c7ccccc7)c7ccccc7c6)cc5)c4c3)c3cccc4c3oc3ccccc34)c2)cc1]

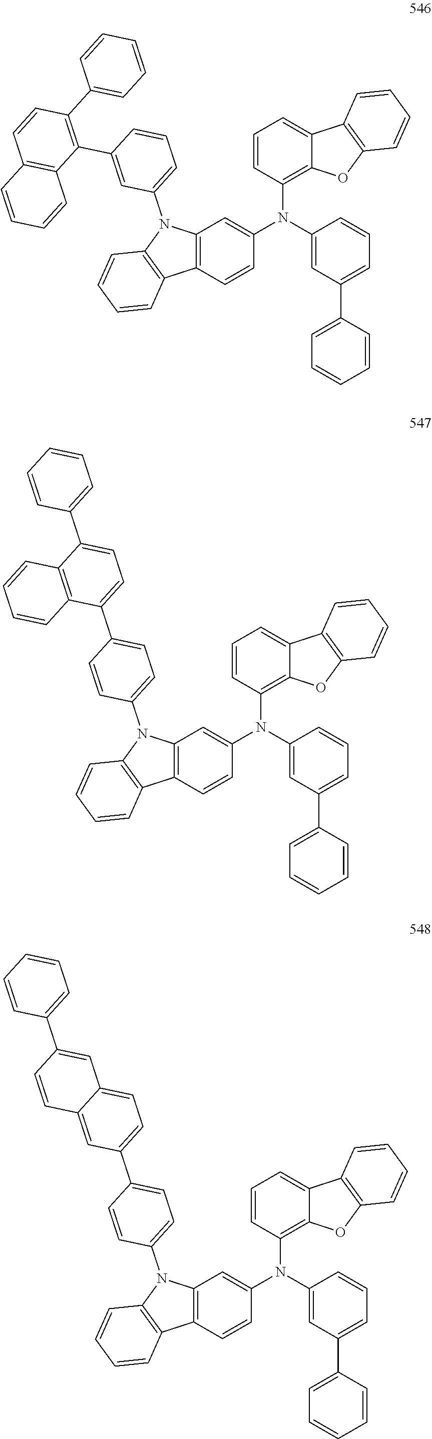 [SMILES: c1ccc(-c2cccc(N(c3ccc4c5ccccc5n(-c5cccc(-c6c(-c7ccccc7)ccc7ccccc67)c5)c4c3)c3cccc4c3oc3ccccc34)c2)cc1 ; c1ccc(-c2cccc(N(c3ccc4c5ccccc5n(-c5ccc(-c6ccc(-c7ccccc7)c7ccccc67)cc5)c4c3)c3cccc4c3oc3ccccc34)c2)cc1 ; c1ccc(-c2cccc(N(c3ccc4c5ccccc5n(-c5ccc(-c6ccc7cc(-c8ccccc8)ccc7c6)cc5)c4c3)c3cccc4c3oc3ccccc34)c2)cc1]

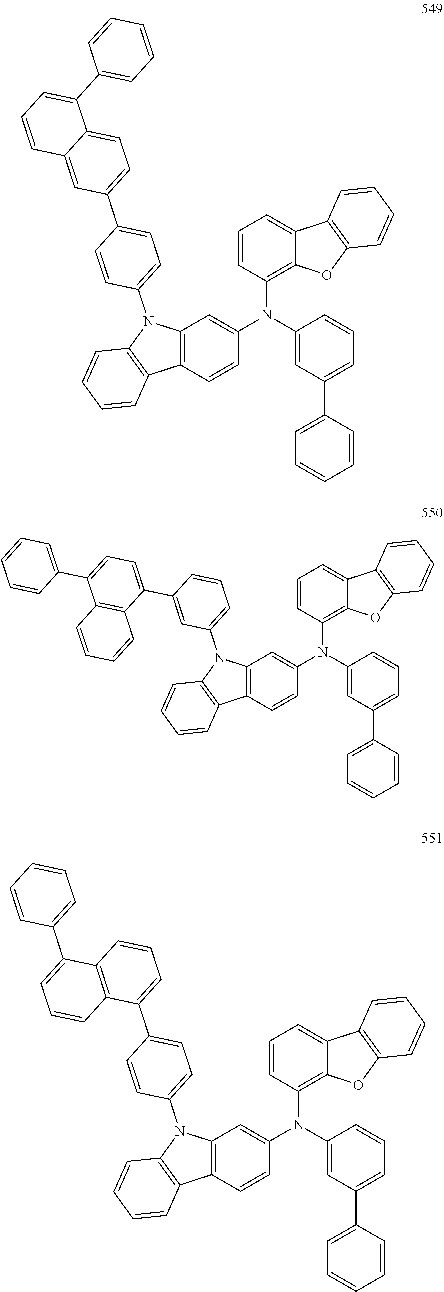 [SMILES: c1ccc(-c2cccc(N(c3ccc4c5ccccc5n(-c5ccc(-c6ccc7c(-c8ccccc8)cccc7c6)cc5)c4c3)c3cccc4c3oc3ccccc34)c2)cc1 ; c1ccc(-c2cccc(N(c3ccc4c5ccccc5n(-c5cccc(-c6ccc(-c7ccccc7)c7ccccc67)c5)c4c3)c3cccc4c3oc3ccccc34)c2)cc1 ; c1ccc(-c2cccc(N(c3ccc4c5ccccc5n(-c5ccc(-c6cccc7c(-c8ccccc8)cccc67)cc5)c4c3)c3cccc4c3oc3ccccc34)c2)cc1]

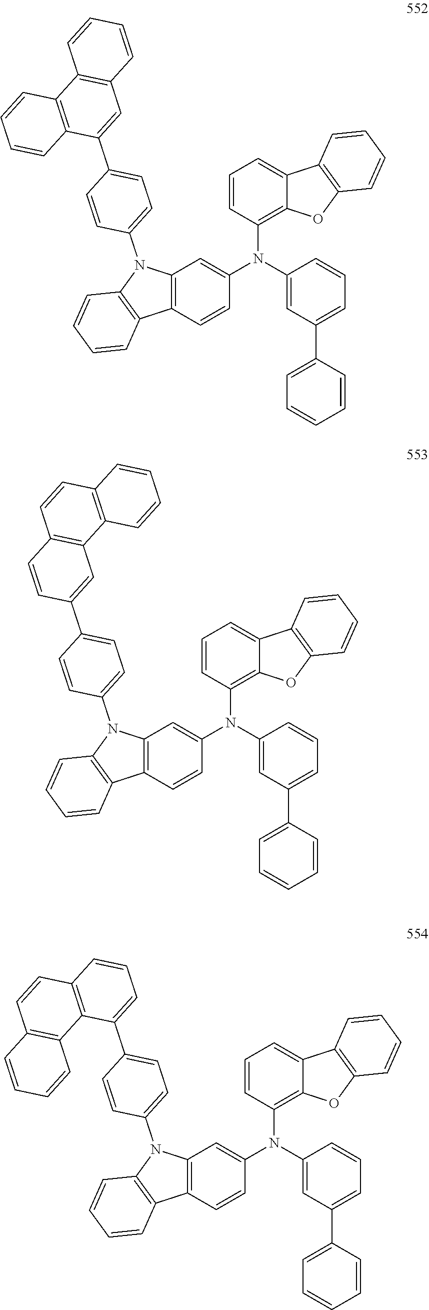 [SMILES: c1ccc(-c2cccc(N(c3ccc4c5ccccc5n(-c5ccc(-c6cc7ccccc7c7ccccc67)cc5)c4c3)c3cccc4c3oc3ccccc34)c2)cc1 ; c1ccc(-c2cccc(N(c3ccc4c5ccccc5n(-c5ccc(-c6ccc7ccc8ccccc8c7c6)cc5)c4c3)c3cccc4c3oc3ccccc34)c2)cc1 ; c1ccc(-c2cccc(N(c3ccc4c5ccccc5n(-c5ccc(-c6cccc7ccc8ccccc8c67)cc5)c4c3)c3cccc4c3oc3ccccc34)c2)cc1]

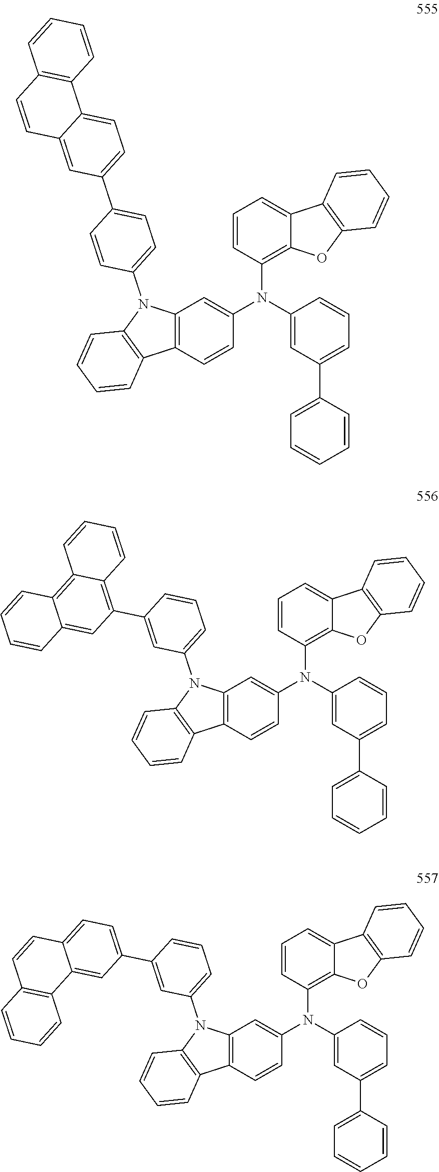 [SMILES: c1ccc(-c2cccc(N(c3ccc4c5ccccc5n(-c5ccc(-c6ccc7c(ccc8ccccc87)c6)cc5)c4c3)c3cccc4c3oc3ccccc34)c2)cc1 ; c1ccc(-c2cccc(N(c3ccc4c5ccccc5n(-c5cccc(-c6cc7ccccc7c7ccccc67)c5)c4c3)c3cccc4c3oc3ccccc34)c2)cc1 ; c1ccc(-c2cccc(N(c3ccc4c5ccccc5n(-c5cccc(-c6ccc7ccc8ccccc8c7c6)c5)c4c3)c3cccc4c3oc3ccccc34)c2)cc1]

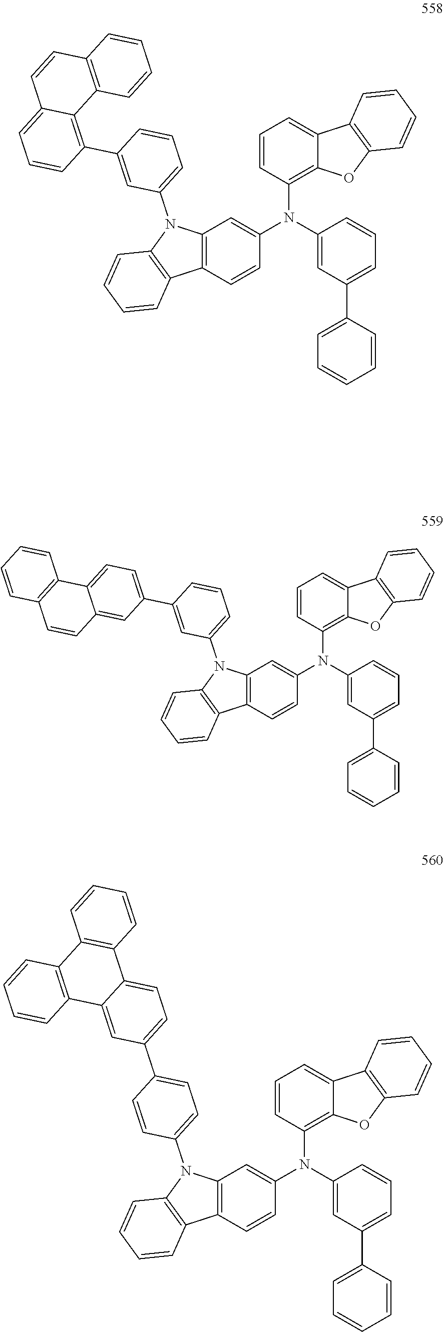 [SMILES: c1ccc(-c2cccc(N(c3ccc4c5ccccc5n(-c5cccc(-c6cccc7ccc8ccccc8c67)c5)c4c3)c3cccc4c3oc3ccccc34)c2)cc1 ; c1ccc(-c2cccc(N(c3ccc4c5ccccc5n(-c5cccc(-c6ccc7c(ccc8ccccc87)c6)c5)c4c3)c3cccc4c3oc3ccccc34)c2)cc1 ; c1ccc(-c2cccc(N(c3ccc4c5ccccc5n(-c5ccc(-c6ccc7c8ccccc8c8ccccc8c7c6)cc5)c4c3)c3cccc4c3oc3ccccc34)c2)cc1]

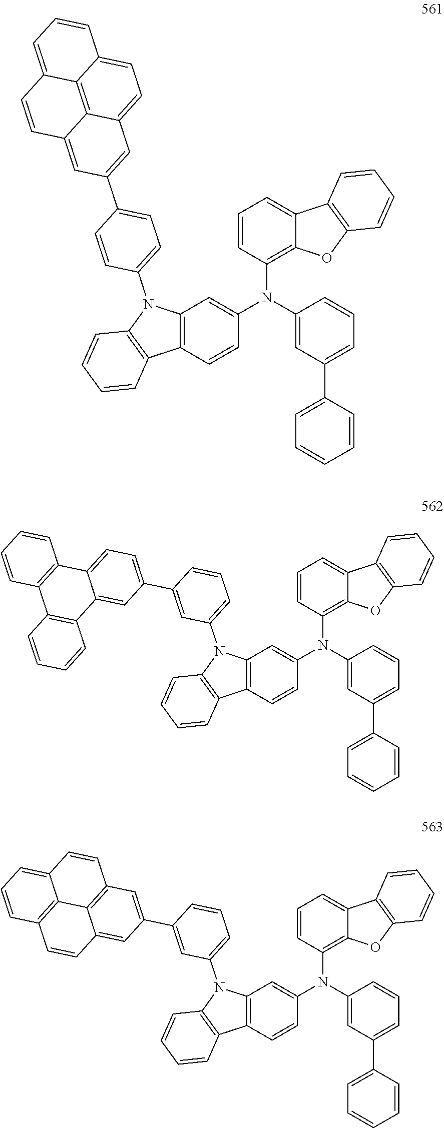 [SMILES: c1ccc(-c2cccc(N(c3ccc4c5ccccc5n(-c5ccc(-c6cc7ccc8cccc9ccc(c6)c7c89)cc5)c4c3)c3cccc4c3oc3ccccc34)c2)cc1 ; c1ccc(-c2cccc(N(c3ccc4c5ccccc5n(-c5cccc(-c6ccc7c8ccccc8c8ccccc8c7c6)c5)c4c3)c3cccc4c3oc3ccccc34)c2)cc1 ; c1ccc(-c2cccc(N(c3ccc4c5ccccc5n(-c5cccc(-c6cc7ccc8cccc9ccc(c6)c7c89)c5)c4c3)c3cccc4c3oc3ccccc34)c2)cc1]

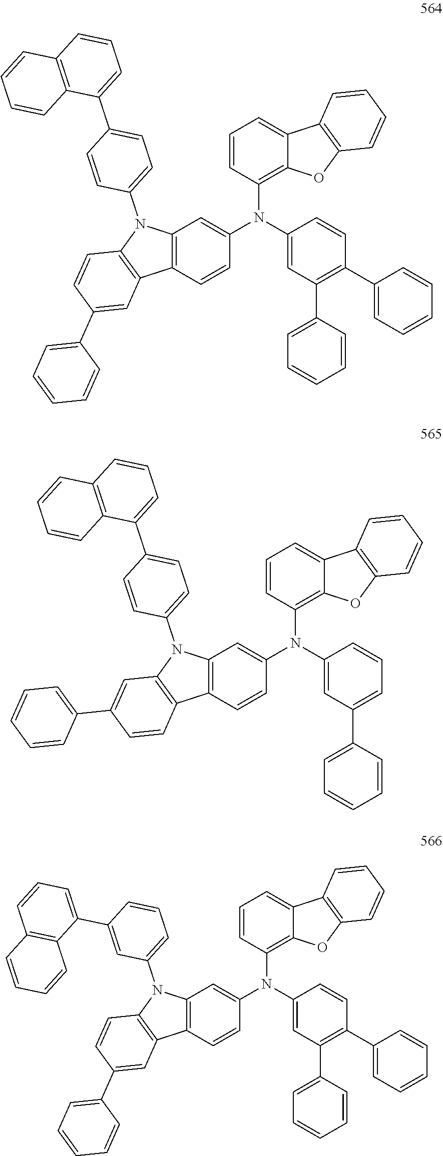 [SMILES: c1ccc(-c2ccc3c(c2)c2ccc(N(c4ccc(-c5ccccc5)c(-c5ccccc5)c4)c4cccc5c4oc4ccccc45)cc2n3-c2ccc(-c3cccc4ccccc34)cc2)cc1 ; c1ccc(-c2cccc(N(c3ccc4c5ccc(-c6ccccc6)cc5n(-c5ccc(-c6cccc7ccccc67)cc5)c4c3)c3cccc4c3oc3ccccc34)c2)cc1 ; c1ccc(-c2ccc3c(c2)c2ccc(N(c4ccc(-c5ccccc5)c(-c5ccccc5)c4)c4cccc5c4oc4ccccc45)cc2n3-c2cccc(-c3cccc4ccccc34)c2)cc1]

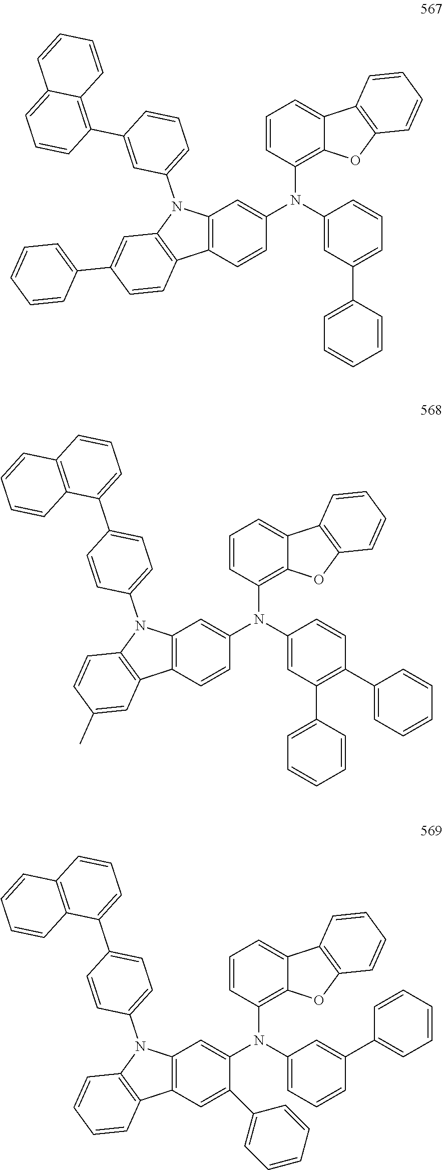 [SMILES: c1ccc(-c2cccc(N(c3ccc4c5ccc(-c6ccccc6)cc5n(-c5cccc(-c6cccc7ccccc67)c5)c4c3)c3cccc4c3oc3ccccc34)c2)cc1 ; Cc1ccc2c(c1)c1ccc(N(c3ccc(-c4ccccc4)c(-c4ccccc4)c3)c3cccc4c3oc3ccccc34)cc1n2-c1ccc(-c2cccc3ccccc23)cc1 ; c1ccc(-c2cccc(N(c3cc4c(cc3-c3ccccc3)c3ccccc3n4-c3ccc(-c4cccc5ccccc45)cc3)c3cccc4c3oc3ccccc34)c2)cc1]

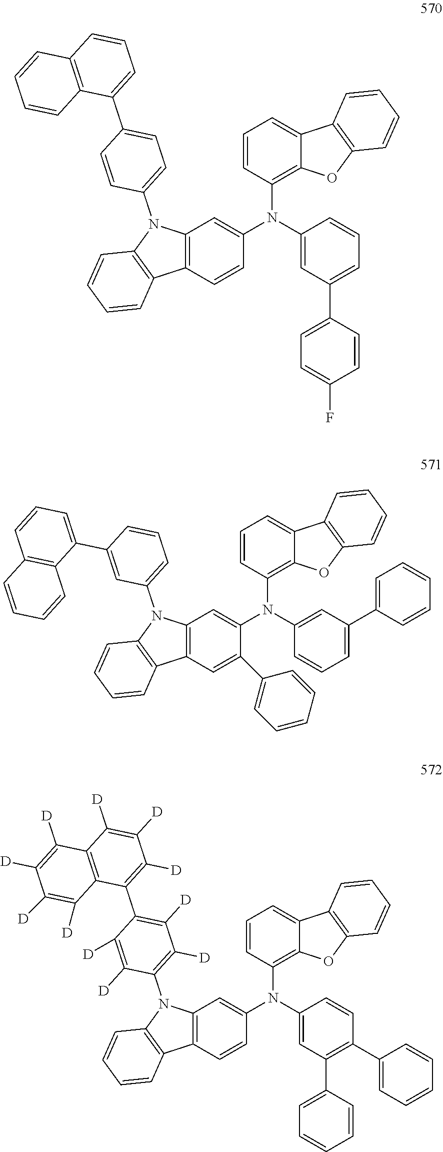 [SMILES: Fc1ccc(-c2cccc(N(c3ccc4c5ccccc5n(-c5ccc(-c6cccc7ccccc67)cc5)c4c3)c3cccc4c3oc3ccccc34)c2)cc1 ; c1ccc(-c2cccc(N(c3cc4c(cc3-c3ccccc3)c3ccccc3n4-c3cccc(-c4cccc5ccccc45)c3)c3cccc4c3oc3ccccc34)c2)cc1 ; [2H]c1c([2H])c(-n2c3ccccc3c3ccc(N(c4ccc(-c5ccccc5)c(-c5ccccc5)c4)c4cccc5c4oc4ccccc45)cc32)c([2H])c([2H])c1-c1c([2H])c([2H])c([2H])c2c([2H])c([2H])c([2H])c([2H])c12]

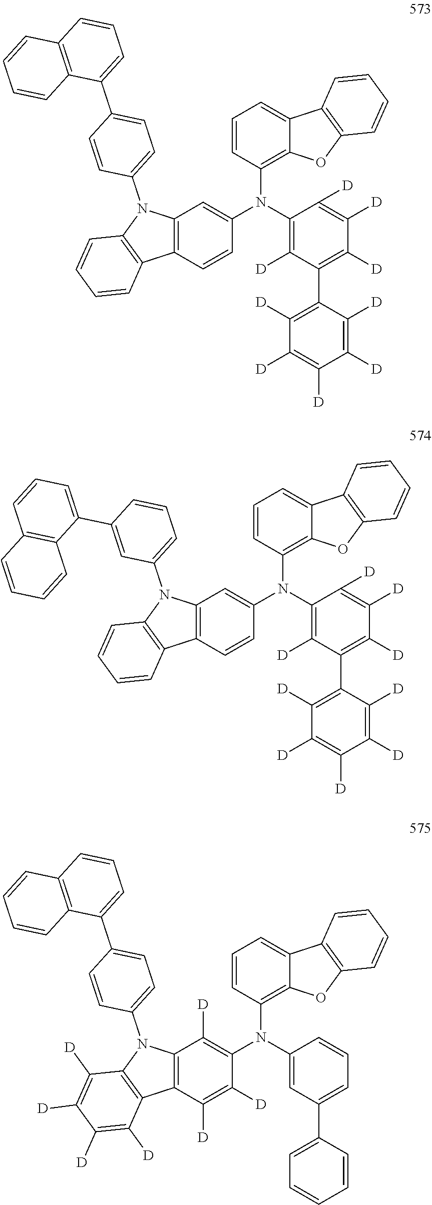 [SMILES: [2H]c1c([2H])c([2H])c(-c2c([2H])c([2H])c([2H])c(N(c3ccc4c5ccccc5n(-c5ccc(-c6cccc7ccccc67)cc5)c4c3)c3cccc4c3oc3ccccc34)c2[2H])c([2H])c1[2H] ; [2H]c1c([2H])c([2H])c(-c2c([2H])c([2H])c([2H])c(N(c3ccc4c5ccccc5n(-c5cccc(-c6cccc7ccccc67)c5)c4c3)c3cccc4c3oc3ccccc34)c2[2H])c([2H])c1[2H] ; [2H]c1c([2H])c([2H])c2c(c1[2H])c1c([2H])c([2H])c(N(c3cccc(-c4ccccc4)c3)c3cccc4c3oc3ccccc34)c([2H])c1n2-c1ccc(-c2cccc3ccccc23)cc1]

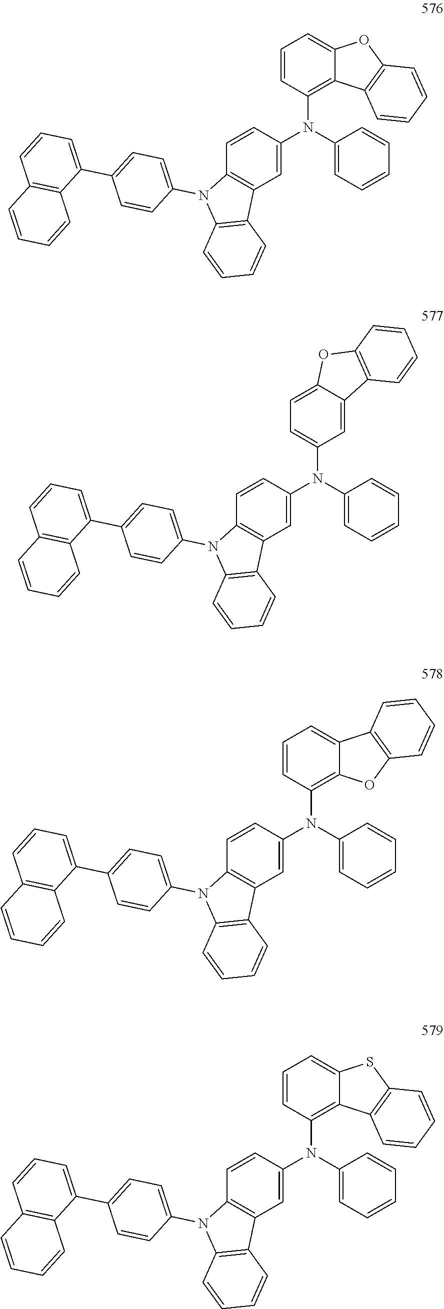 [SMILES: c1ccc(N(c2ccc3c(c2)c2ccccc2n3-c2ccc(-c3cccc4ccccc34)cc2)c2cccc3oc4ccccc4c23)cc1 ; c1ccc(N(c2ccc3oc4ccccc4c3c2)c2ccc3c(c2)c2ccccc2n3-c2ccc(-c3cccc4ccccc34)cc2)cc1 ; c1ccc(N(c2ccc3c(c2)c2ccccc2n3-c2ccc(-c3cccc4ccccc34)cc2)c2cccc3c2oc2ccccc23)cc1 ; c1ccc(N(c2ccc3c(c2)c2ccccc2n3-c2ccc(-c3cccc4ccccc34)cc2)c2cccc3sc4ccccc4c23)cc1]

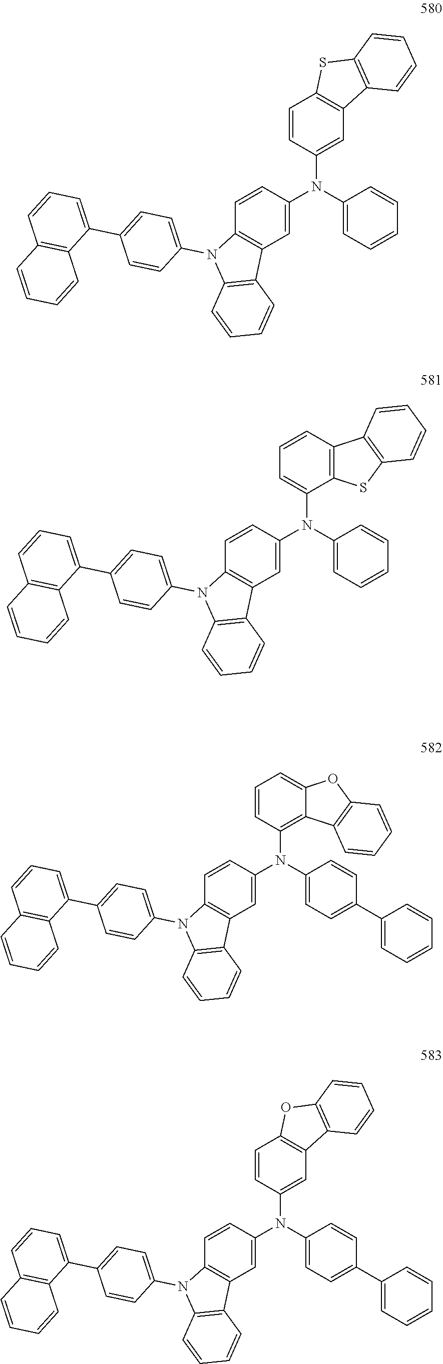 [SMILES: c1ccc(N(c2ccc3sc4ccccc4c3c2)c2ccc3c(c2)c2ccccc2n3-c2ccc(-c3cccc4ccccc34)cc2)cc1 ; c1ccc(N(c2ccc3c(c2)c2ccccc2n3-c2ccc(-c3cccc4ccccc34)cc2)c2cccc3c2sc2ccccc23)cc1 ; c1ccc(-c2ccc(N(c3ccc4c(c3)c3ccccc3n4-c3ccc(-c4cccc5ccccc45)cc3)c3cccc4oc5ccccc5c34)cc2)cc1 ; c1ccc(-c2ccc(N(c3ccc4oc5ccccc5c4c3)c3ccc4c(c3)c3ccccc3n4-c3ccc(-c4cccc5ccccc45)cc3)cc2)cc1]

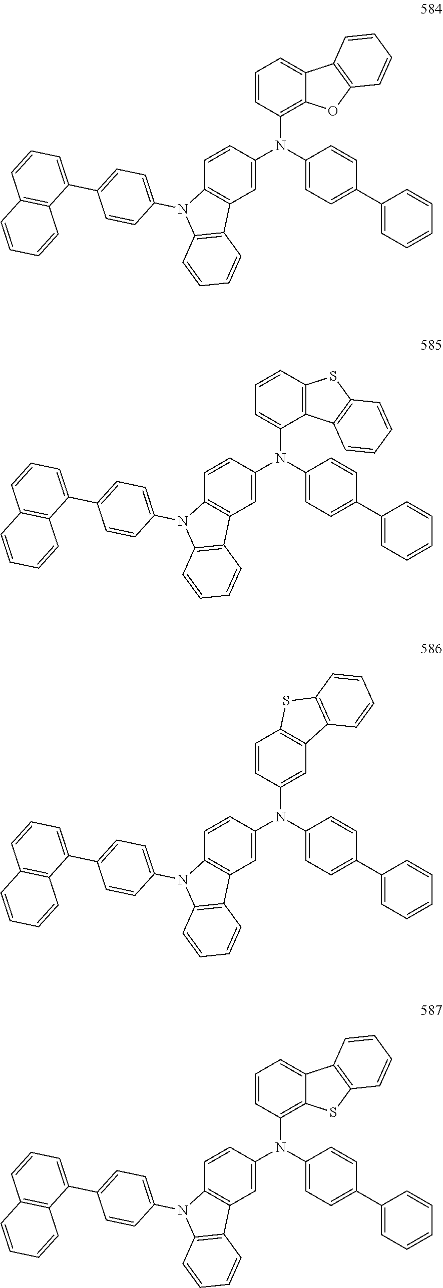 [SMILES: c1ccc(-c2ccc(N(c3ccc4c(c3)c3ccccc3n4-c3ccc(-c4cccc5ccccc45)cc3)c3cccc4c3oc3ccccc34)cc2)cc1 ; c1ccc(-c2ccc(N(c3ccc4c(c3)c3ccccc3n4-c3ccc(-c4cccc5ccccc45)cc3)c3cccc4sc5ccccc5c34)cc2)cc1 ; c1ccc(-c2ccc(N(c3ccc4sc5ccccc5c4c3)c3ccc4c(c3)c3ccccc3n4-c3ccc(-c4cccc5ccccc45)cc3)cc2)cc1 ; c1ccc(-c2ccc(N(c3ccc4c(c3)c3ccccc3n4-c3ccc(-c4cccc5ccccc45)cc3)c3cccc4c3sc3ccccc34)cc2)cc1]

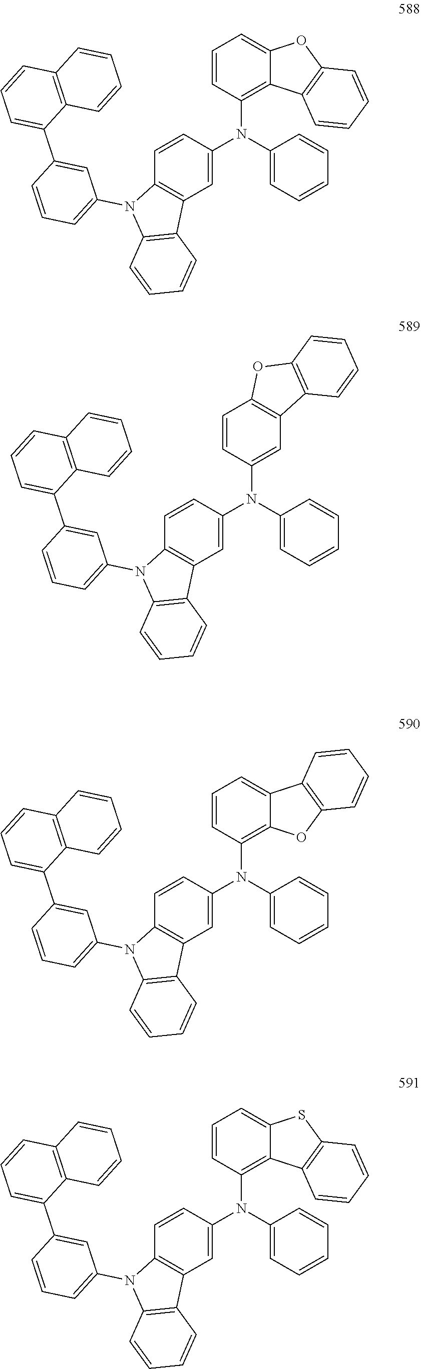 [SMILES: c1ccc(N(c2ccc3c(c2)c2ccccc2n3-c2cccc(-c3cccc4ccccc34)c2)c2cccc3oc4ccccc4c23)cc1 ; c1ccc(N(c2ccc3oc4ccccc4c3c2)c2ccc3c(c2)c2ccccc2n3-c2cccc(-c3cccc4ccccc34)c2)cc1 ; c1ccc(N(c2ccc3c(c2)c2ccccc2n3-c2cccc(-c3cccc4ccccc34)c2)c2cccc3c2oc2ccccc23)cc1 ; c1ccc(N(c2ccc3c(c2)c2ccccc2n3-c2cccc(-c3cccc4ccccc34)c2)c2cccc3sc4ccccc4c23)cc1]

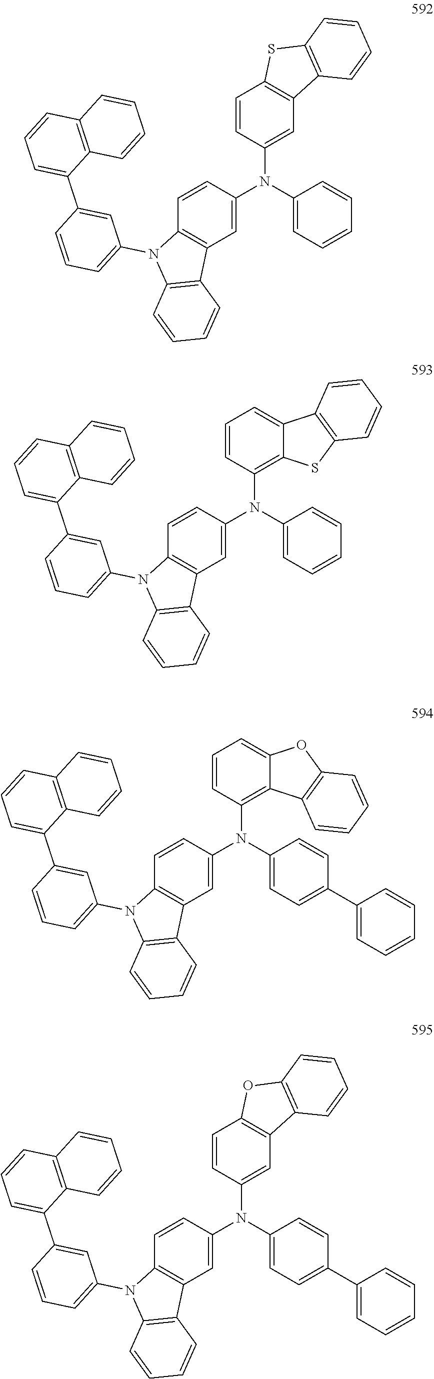 [SMILES: c1ccc(N(c2ccc3sc4ccccc4c3c2)c2ccc3c(c2)c2ccccc2n3-c2cccc(-c3cccc4ccccc34)c2)cc1 ; c1ccc(N(c2ccc3c(c2)c2ccccc2n3-c2cccc(-c3cccc4ccccc34)c2)c2cccc3c2sc2ccccc23)cc1 ; c1ccc(-c2ccc(N(c3ccc4c(c3)c3ccccc3n4-c3cccc(-c4cccc5ccccc45)c3)c3cccc4oc5ccccc5c34)cc2)cc1 ; c1ccc(-c2ccc(N(c3ccc4oc5ccccc5c4c3)c3ccc4c(c3)c3ccccc3n4-c3cccc(-c4cccc5ccccc45)c3)cc2)cc1]

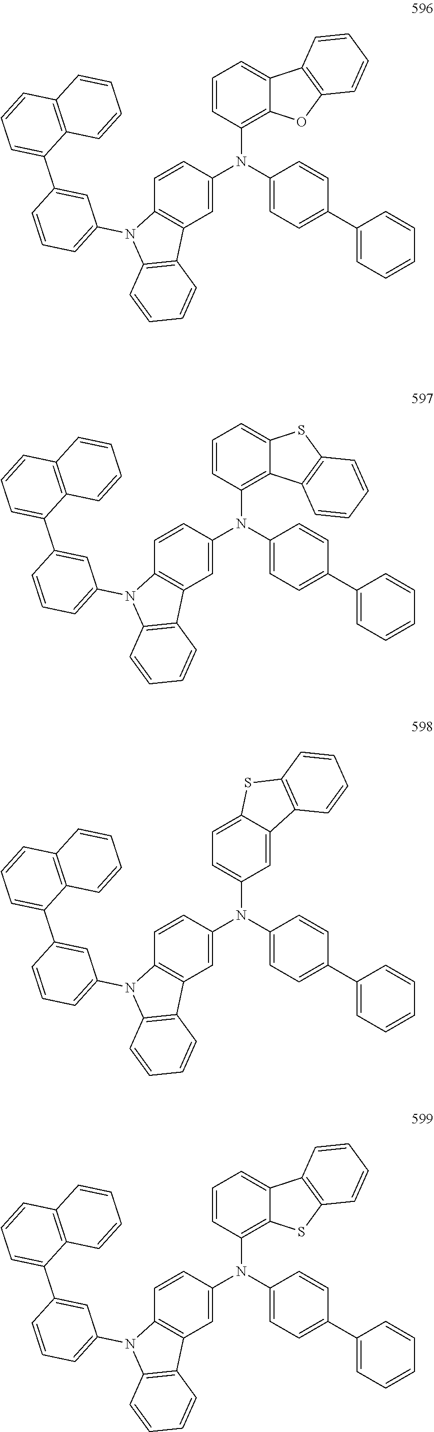 [SMILES: c1ccc(-c2ccc(N(c3ccc4c(c3)c3ccccc3n4-c3cccc(-c4cccc5ccccc45)c3)c3cccc4c3oc3ccccc34)cc2)cc1 ; c1ccc(-c2ccc(N(c3ccc4c(c3)c3ccccc3n4-c3cccc(-c4cccc5ccccc45)c3)c3cccc4sc5ccccc5c34)cc2)cc1 ; c1ccc(-c2ccc(N(c3ccc4sc5ccccc5c4c3)c3ccc4c(c3)c3ccccc3n4-c3cccc(-c4cccc5ccccc45)c3)cc2)cc1 ; c1ccc(-c2ccc(N(c3ccc4c(c3)c3ccccc3n4-c3cccc(-c4cccc5ccccc45)c3)c3cccc4c3sc3ccccc34)cc2)cc1]

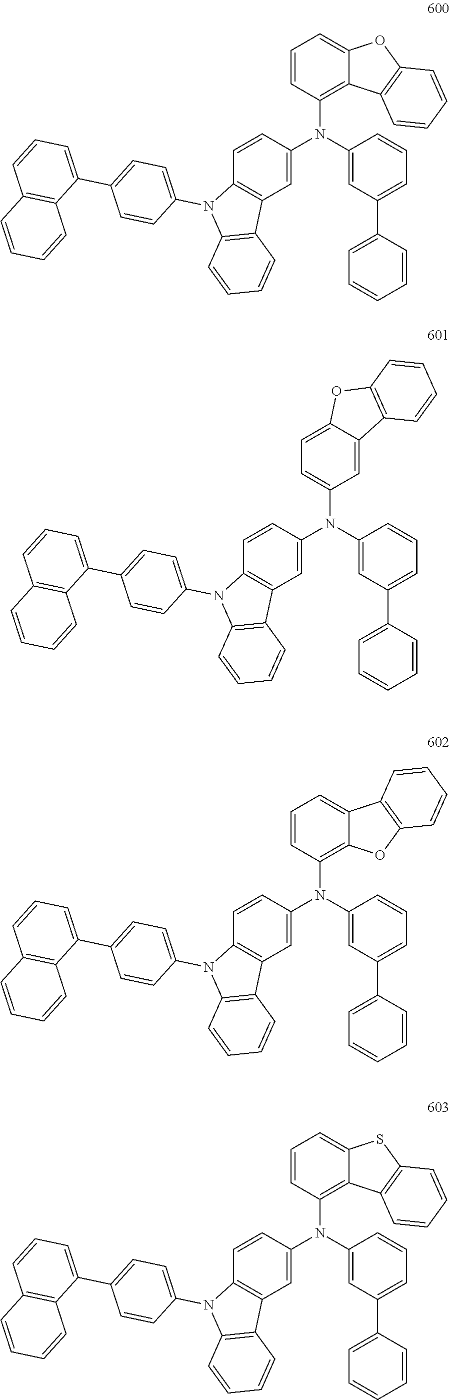 [SMILES: c1ccc(-c2cccc(N(c3ccc4c(c3)c3ccccc3n4-c3ccc(-c4cccc5ccccc45)cc3)c3cccc4oc5ccccc5c34)c2)cc1 ; c1ccc(-c2cccc(N(c3ccc4oc5ccccc5c4c3)c3ccc4c(c3)c3ccccc3n4-c3ccc(-c4cccc5ccccc45)cc3)c2)cc1 ; c1ccc(-c2cccc(N(c3ccc4c(c3)c3ccccc3n4-c3ccc(-c4cccc5ccccc45)cc3)c3cccc4c3oc3ccccc34)c2)cc1 ; c1ccc(-c2cccc(N(c3ccc4c(c3)c3ccccc3n4-c3ccc(-c4cccc5ccccc45)cc3)c3cccc4sc5ccccc5c34)c2)cc1]

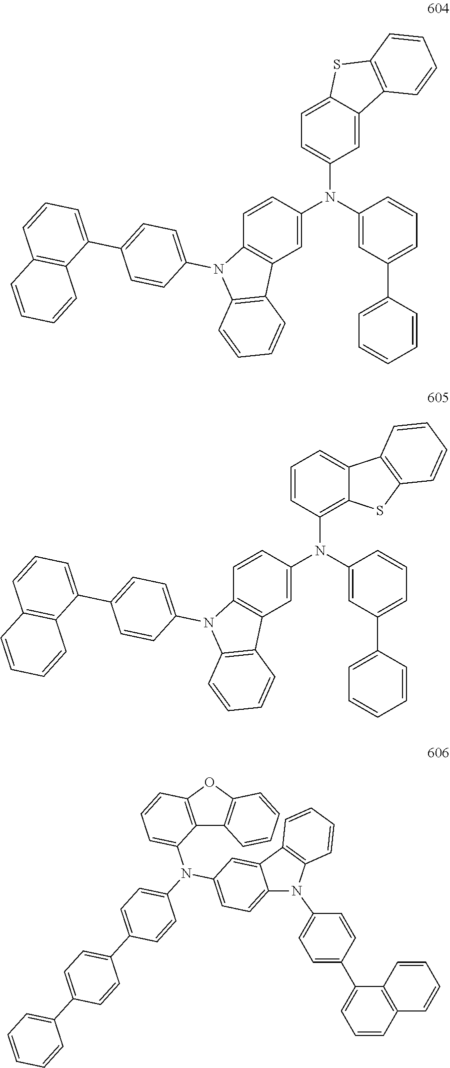 [SMILES: c1ccc(-c2cccc(N(c3ccc4sc5ccccc5c4c3)c3ccc4c(c3)c3ccccc3n4-c3ccc(-c4cccc5ccccc45)cc3)c2)cc1 ; c1ccc(-c2cccc(N(c3ccc4c(c3)c3ccccc3n4-c3ccc(-c4cccc5ccccc45)cc3)c3cccc4c3sc3ccccc34)c2)cc1 ; c1ccc(-c2ccc(-c3ccc(N(c4ccc5c(c4)c4ccccc4n5-c4ccc(-c5cccc6ccccc56)cc4)c4cccc5oc6ccccc6c45)cc3)cc2)cc1]

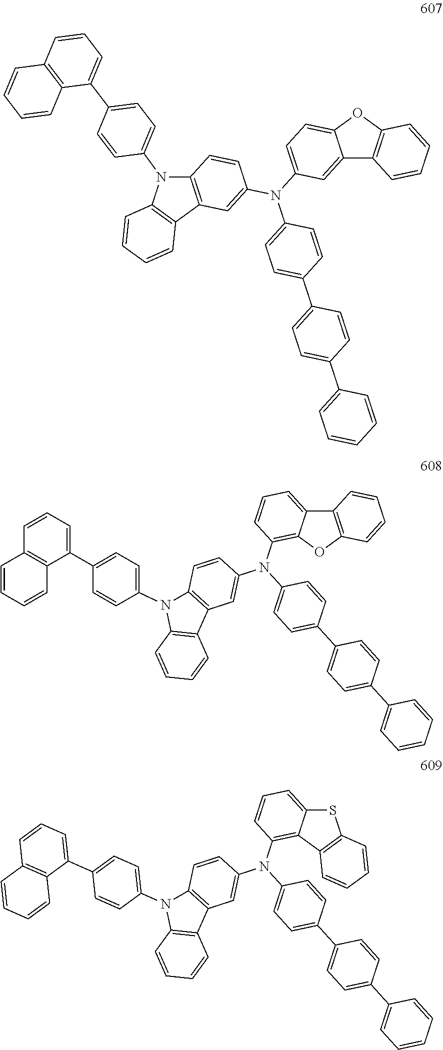 [SMILES: c1ccc(-c2ccc(-c3ccc(N(c4ccc5oc6ccccc6c5c4)c4ccc5c(c4)c4ccccc4n5-c4ccc(-c5cccc6ccccc56)cc4)cc3)cc2)cc1 ; c1ccc(-c2ccc(-c3ccc(N(c4ccc5c(c4)c4ccccc4n5-c4ccc(-c5cccc6ccccc56)cc4)c4cccc5c4oc4ccccc45)cc3)cc2)cc1 ; c1ccc(-c2ccc(-c3ccc(N(c4ccc5c(c4)c4ccccc4n5-c4ccc(-c5cccc6ccccc56)cc4)c4cccc5sc6ccccc6c45)cc3)cc2)cc1]

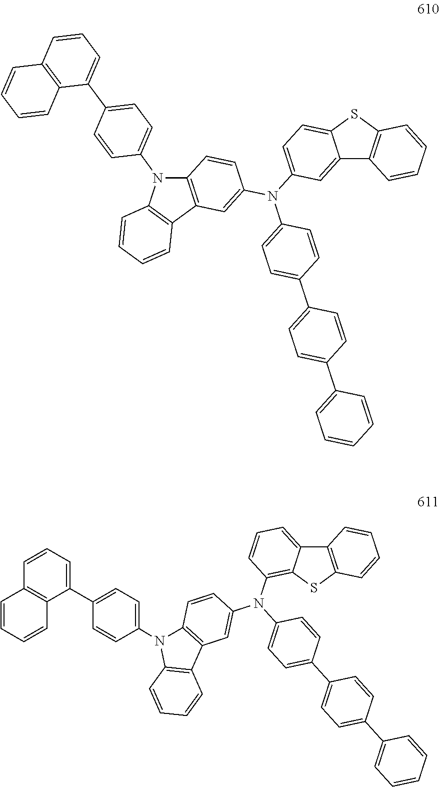 [SMILES: c1ccc(-c2ccc(-c3ccc(N(c4ccc5sc6ccccc6c5c4)c4ccc5c(c4)c4ccccc4n5-c4ccc(-c5cccc6ccccc56)cc4)cc3)cc2)cc1 ; c1ccc(-c2ccc(-c3ccc(N(c4ccc5c(c4)c4ccccc4n5-c4ccc(-c5cccc6ccccc56)cc4)c4cccc5c4sc4ccccc45)cc3)cc2)cc1]

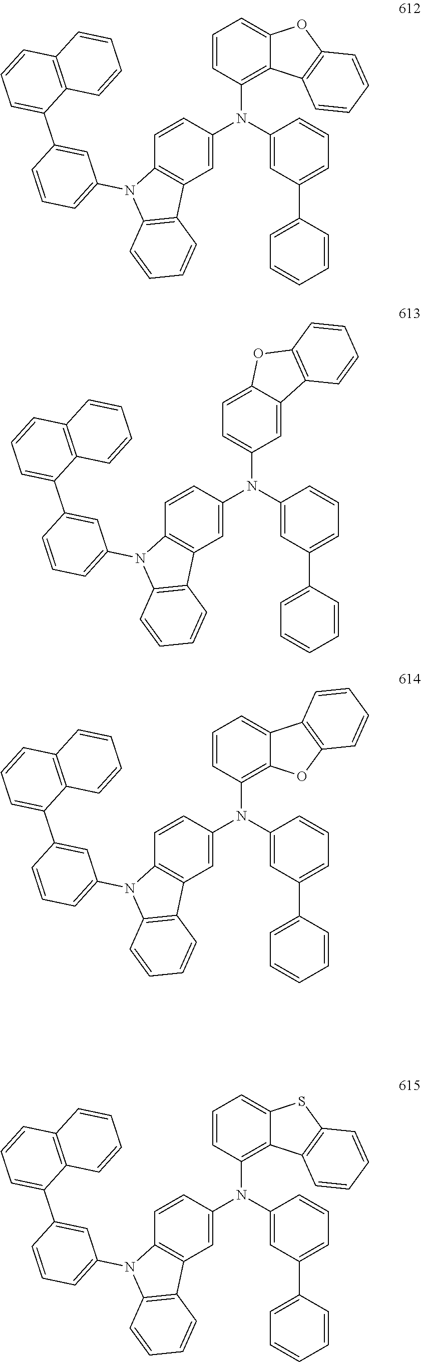

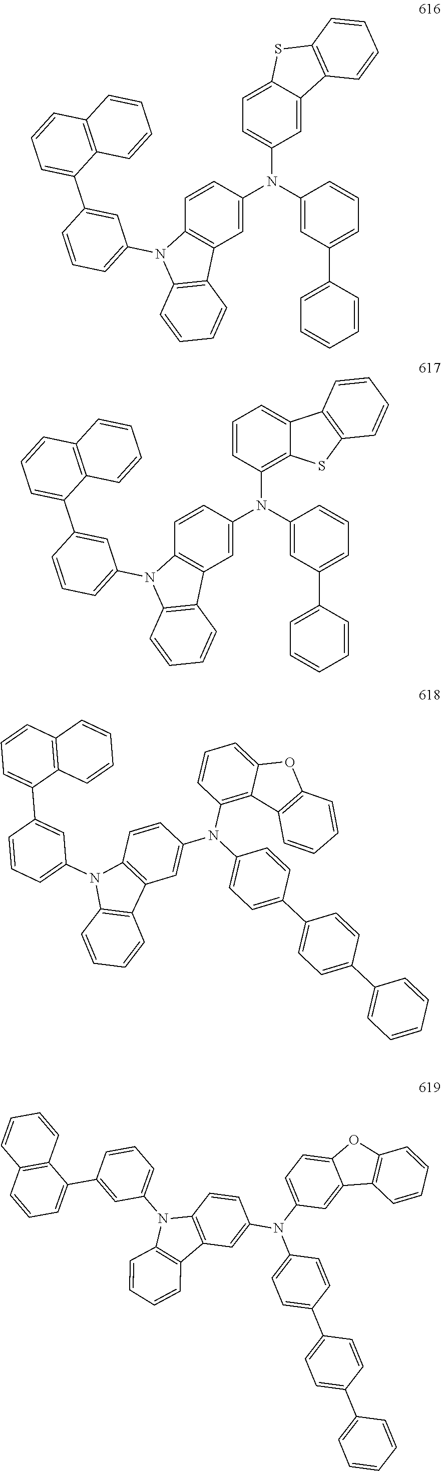

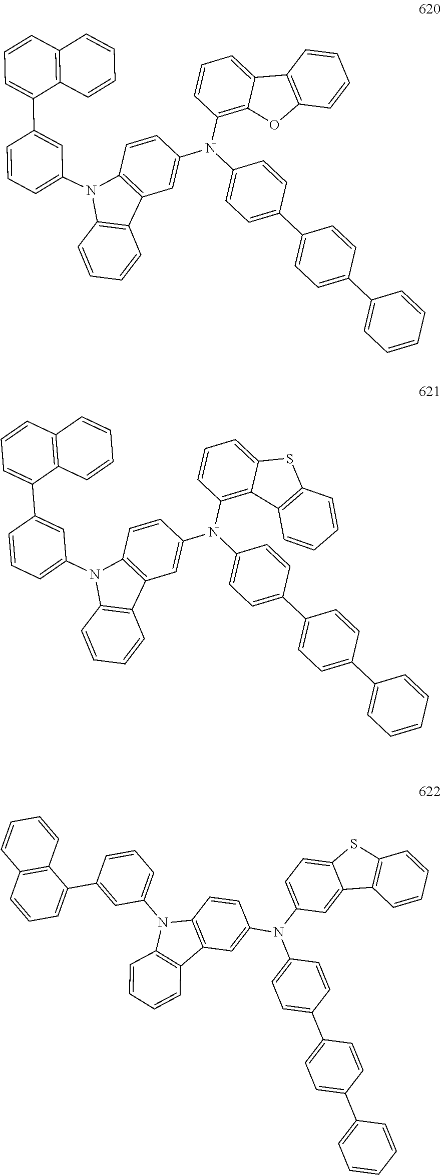 [SMILES: c1ccc(-c2ccc(-c3ccc(N(c4ccc5c(c4)c4ccccc4n5-c4cccc(-c5cccc6ccccc56)c4)c4cccc5c4oc4ccccc45)cc3)cc2)cc1 ; c1ccc(-c2ccc(-c3ccc(N(c4ccc5c(c4)c4ccccc4n5-c4cccc(-c5cccc6ccccc56)c4)c4cccc5sc6ccccc6c45)cc3)cc2)cc1 ; c1ccc(-c2ccc(-c3ccc(N(c4ccc5sc6ccccc6c5c4)c4ccc5c(c4)c4ccccc4n5-c4cccc(-c5cccc6ccccc56)c4)cc3)cc2)cc1]

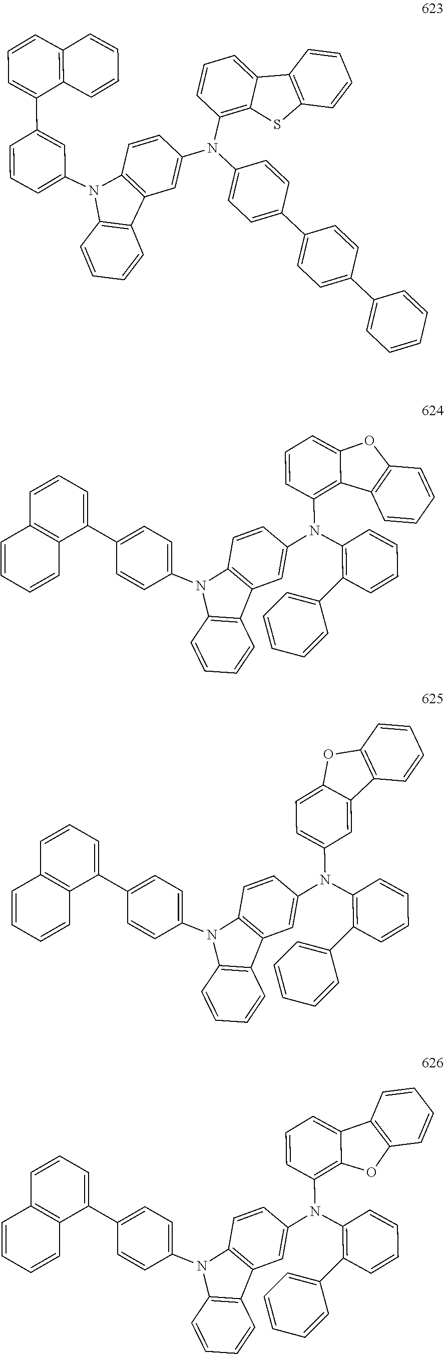 [SMILES: c1ccc(-c2ccc(-c3ccc(N(c4ccc5c(c4)c4ccccc4n5-c4cccc(-c5cccc6ccccc56)c4)c4cccc5c4sc4ccccc45)cc3)cc2)cc1 ; c1ccc(-c2ccccc2N(c2ccc3c(c2)c2ccccc2n3-c2ccc(-c3cccc4ccccc34)cc2)c2cccc3oc4ccccc4c23)cc1 ; c1ccc(-c2ccccc2N(c2ccc3oc4ccccc4c3c2)c2ccc3c(c2)c2ccccc2n3-c2ccc(-c3cccc4ccccc34)cc2)cc1 ; c1ccc(-c2ccccc2N(c2ccc3c(c2)c2ccccc2n3-c2ccc(-c3cccc4ccccc34)cc2)c2cccc3c2oc2ccccc23)cc1]

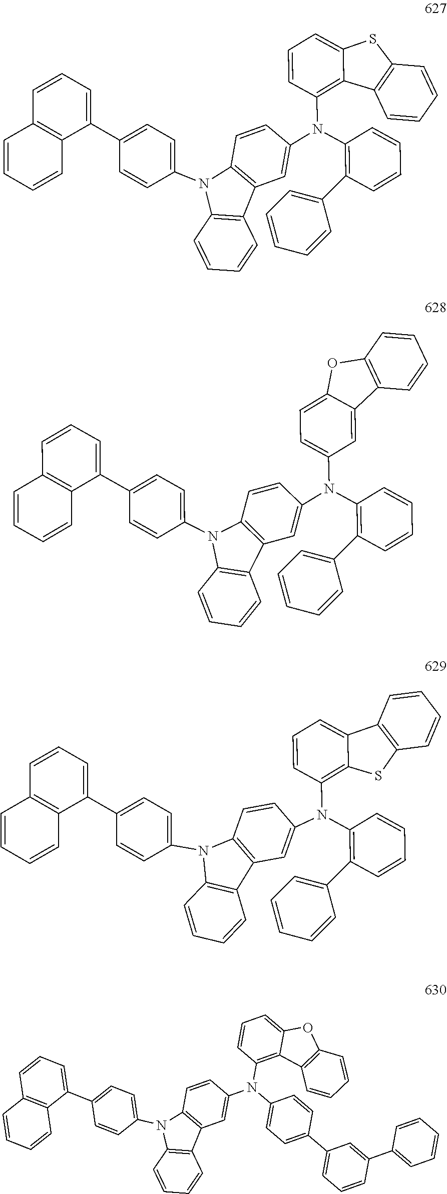 [SMILES: c1ccc(-c2ccccc2N(c2ccc3c(c2)c2ccccc2n3-c2ccc(-c3cccc4ccccc34)cc2)c2cccc3sc4ccccc4c23)cc1 ; c1ccc(-c2ccccc2N(c2ccc3oc4ccccc4c3c2)c2ccc3c(c2)c2ccccc2n3-c2ccc(-c3cccc4ccccc34)cc2)cc1 ; c1ccc(-c2ccccc2N(c2ccc3c(c2)c2ccccc2n3-c2ccc(-c3cccc4ccccc34)cc2)c2cccc3c2sc2ccccc23)cc1 ; c1ccc(-c2cccc(-c3ccc(N(c4ccc5c(c4)c4ccccc4n5-c4ccc(-c5cccc6ccccc56)cc4)c4cccc5oc6ccccc6c45)cc3)c2)cc1]

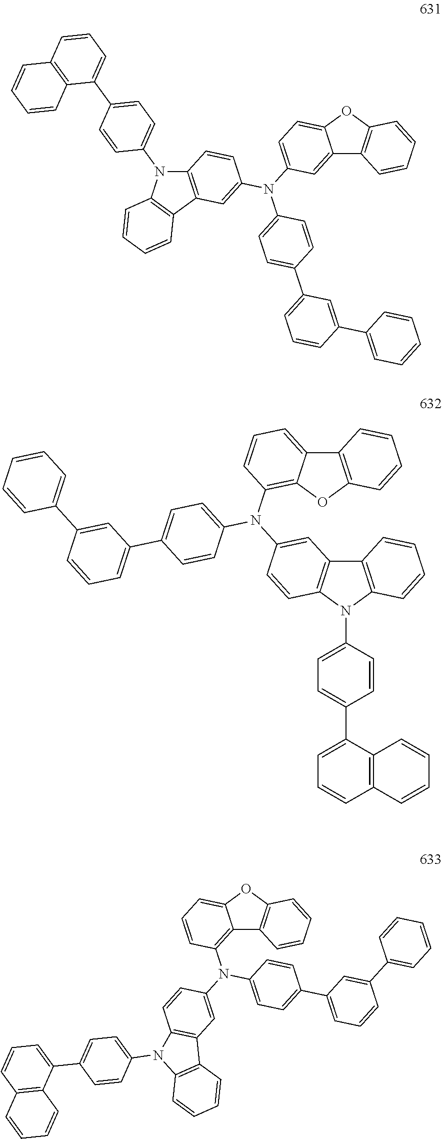 [SMILES: c1ccc(-c2cccc(-c3ccc(N(c4ccc5oc6ccccc6c5c4)c4ccc5c(c4)c4ccccc4n5-c4ccc(-c5cccc6ccccc56)cc4)cc3)c2)cc1 ; c1ccc(-c2cccc(-c3ccc(N(c4ccc5c(c4)c4ccccc4n5-c4ccc(-c5cccc6ccccc56)cc4)c4cccc5c4oc4ccccc45)cc3)c2)cc1 ; c1ccc(-c2cccc(-c3ccc(N(c4ccc5c(c4)c4ccccc4n5-c4ccc(-c5cccc6ccccc56)cc4)c4cccc5oc6ccccc6c45)cc3)c2)cc1]

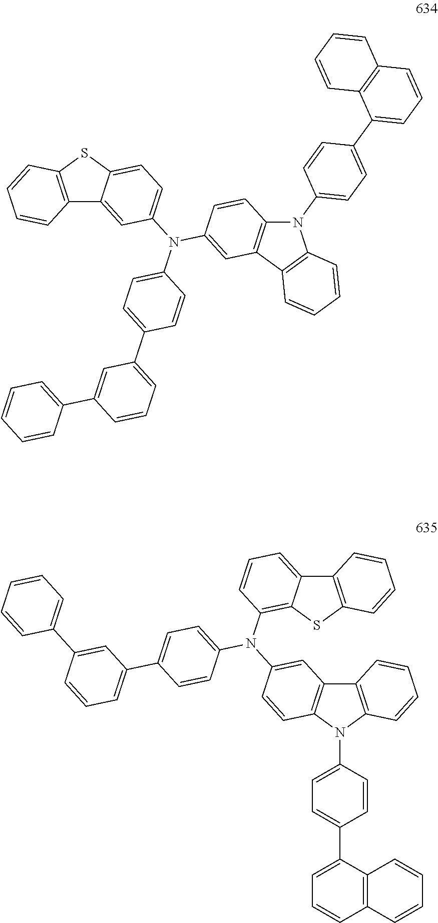 [SMILES: c1ccc(-c2cccc(-c3ccc(N(c4ccc5sc6ccccc6c5c4)c4ccc5c(c4)c4ccccc4n5-c4ccc(-c5cccc6ccccc56)cc4)cc3)c2)cc1 ; c1ccc(-c2cccc(-c3ccc(N(c4ccc5c(c4)c4ccccc4n5-c4ccc(-c5cccc6ccccc56)cc4)c4cccc5c4sc4ccccc45)cc3)c2)cc1]

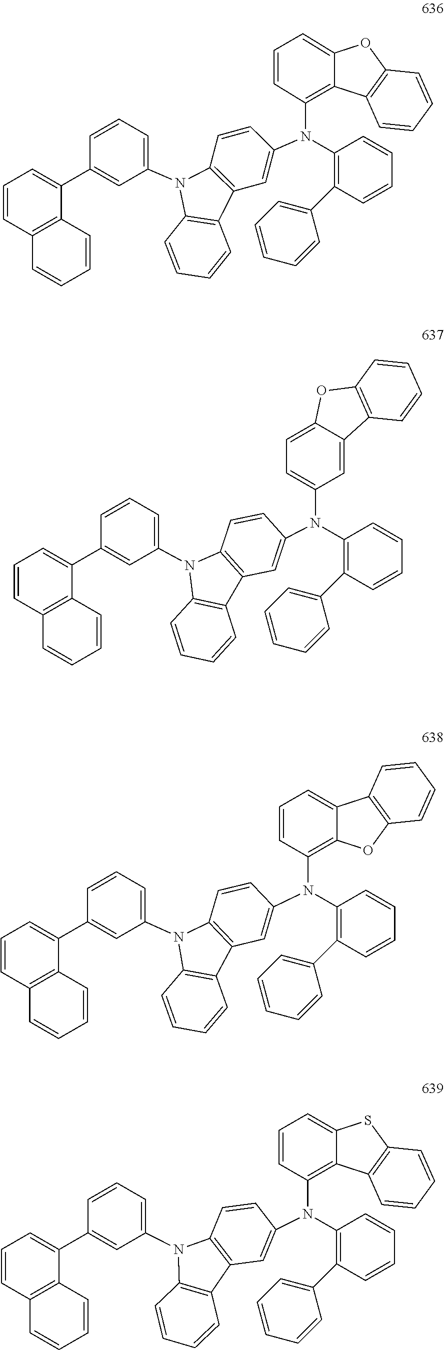 [SMILES: c1ccc(-c2ccccc2N(c2ccc3c(c2)c2ccccc2n3-c2cccc(-c3cccc4ccccc34)c2)c2cccc3oc4ccccc4c23)cc1 ; c1ccc(-c2ccccc2N(c2ccc3oc4ccccc4c3c2)c2ccc3c(c2)c2ccccc2n3-c2cccc(-c3cccc4ccccc34)c2)cc1 ; c1ccc(-c2ccccc2N(c2ccc3c(c2)c2ccccc2n3-c2cccc(-c3cccc4ccccc34)c2)c2cccc3c2oc2ccccc23)cc1 ; c1ccc(-c2ccccc2N(c2ccc3c(c2)c2ccccc2n3-c2cccc(-c3cccc4ccccc34)c2)c2cccc3sc4ccccc4c23)cc1]

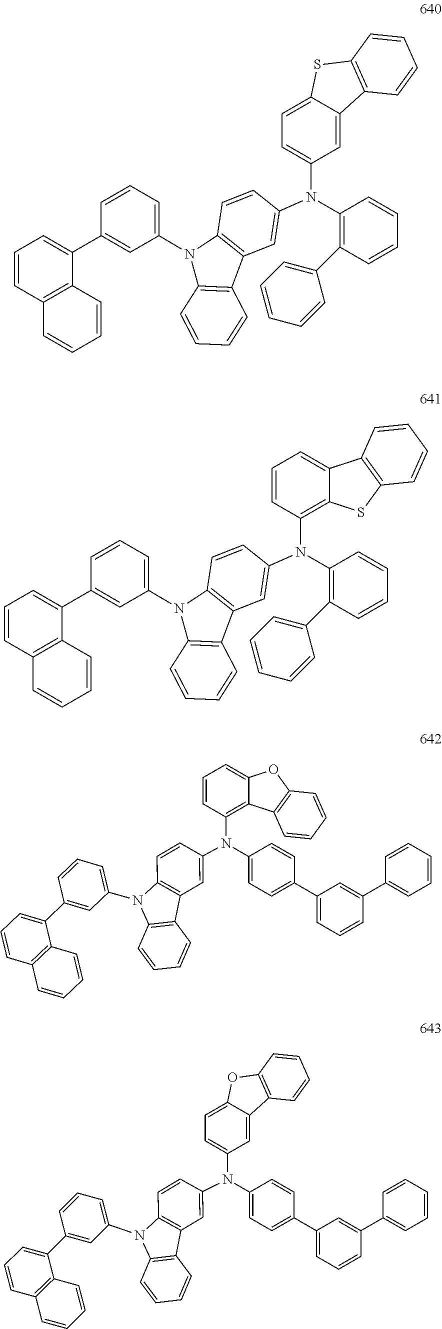 [SMILES: c1ccc(-c2ccccc2N(c2ccc3sc4ccccc4c3c2)c2ccc3c(c2)c2ccccc2n3-c2cccc(-c3cccc4ccccc34)c2)cc1 ; c1ccc(-c2ccccc2N(c2ccc3c(c2)c2ccccc2n3-c2cccc(-c3cccc4ccccc34)c2)c2cccc3c2sc2ccccc23)cc1 ; c1ccc(-c2cccc(-c3ccc(N(c4ccc5c(c4)c4ccccc4n5-c4cccc(-c5cccc6ccccc56)c4)c4cccc5oc6ccccc6c45)cc3)c2)cc1 ; c1ccc(-c2cccc(-c3ccc(N(c4ccc5oc6ccccc6c5c4)c4ccc5c(c4)c4ccccc4n5-c4cccc(-c5cccc6ccccc56)c4)cc3)c2)cc1]

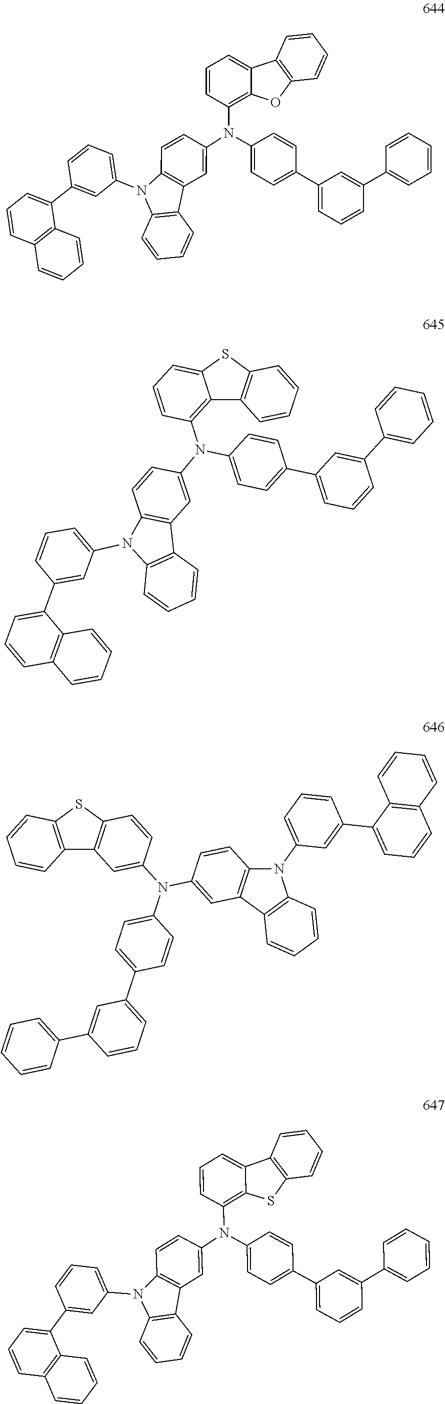 [SMILES: c1ccc(-c2cccc(-c3ccc(N(c4ccc5c(c4)c4ccccc4n5-c4cccc(-c5cccc6ccccc56)c4)c4cccc5c4oc4ccccc45)cc3)c2)cc1 ; c1ccc(-c2cccc(-c3ccc(N(c4ccc5c(c4)c4ccccc4n5-c4cccc(-c5cccc6ccccc56)c4)c4cccc5sc6ccccc6c45)cc3)c2)cc1 ; c1ccc(-c2cccc(-c3ccc(N(c4ccc5sc6ccccc6c5c4)c4ccc5c(c4)c4ccccc4n5-c4cccc(-c5cccc6ccccc56)c4)cc3)c2)cc1 ; c1ccc(-c2cccc(-c3ccc(N(c4ccc5c(c4)c4ccccc4n5-c4cccc(-c5cccc6ccccc56)c4)c4cccc5c4sc4ccccc45)cc3)c2)cc1]

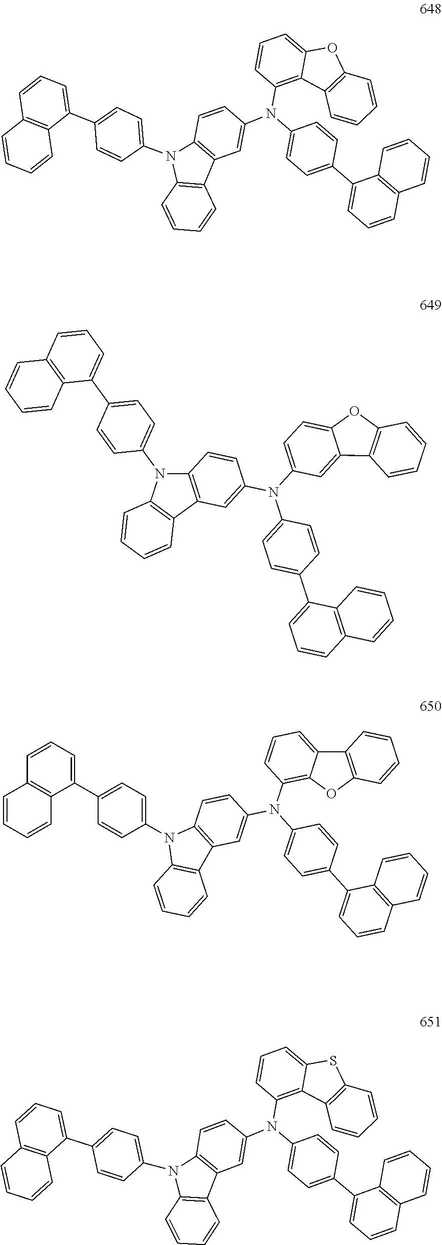 [SMILES: c1ccc2c(-c3ccc(N(c4ccc5c(c4)c4ccccc4n5-c4ccc(-c5cccc6ccccc56)cc4)c4cccc5oc6ccccc6c45)cc3)cccc2c1 ; c1ccc2c(-c3ccc(N(c4ccc5oc6ccccc6c5c4)c4ccc5c(c4)c4ccccc4n5-c4ccc(-c5cccc6ccccc56)cc4)cc3)cccc2c1 ; c1ccc2c(-c3ccc(N(c4ccc5c(c4)c4ccccc4n5-c4ccc(-c5cccc6ccccc56)cc4)c4cccc5c4oc4ccccc45)cc3)cccc2c1 ; c1ccc2c(-c3ccc(N(c4ccc5c(c4)c4ccccc4n5-c4ccc(-c5cccc6ccccc56)cc4)c4cccc5sc6ccccc6c45)cc3)cccc2c1]

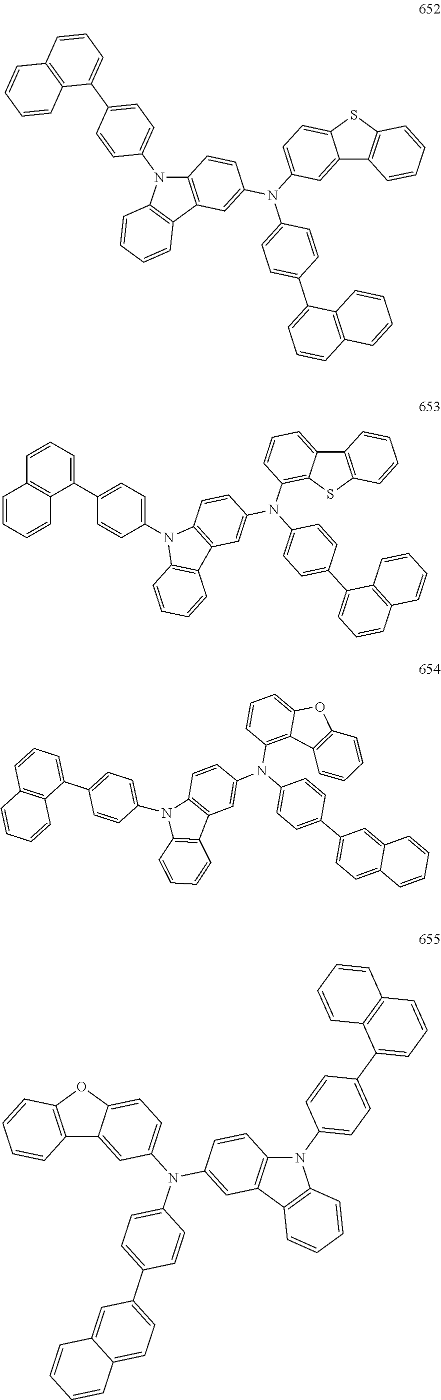 [SMILES: c1ccc2c(-c3ccc(N(c4ccc5sc6ccccc6c5c4)c4ccc5c(c4)c4ccccc4n5-c4ccc(-c5cccc6ccccc56)cc4)cc3)cccc2c1 ; c1ccc2c(-c3ccc(N(c4ccc5c(c4)c4ccccc4n5-c4ccc(-c5cccc6ccccc56)cc4)c4cccc5c4sc4ccccc45)cc3)cccc2c1 ; c1ccc2cc(-c3ccc(N(c4ccc5c(c4)c4ccccc4n5-c4ccc(-c5cccc6ccccc56)cc4)c4cccc5oc6ccccc6c45)cc3)ccc2c1 ; c1ccc2cc(-c3ccc(N(c4ccc5oc6ccccc6c5c4)c4ccc5c(c4)c4ccccc4n5-c4ccc(-c5cccc6ccccc56)cc4)cc3)ccc2c1]

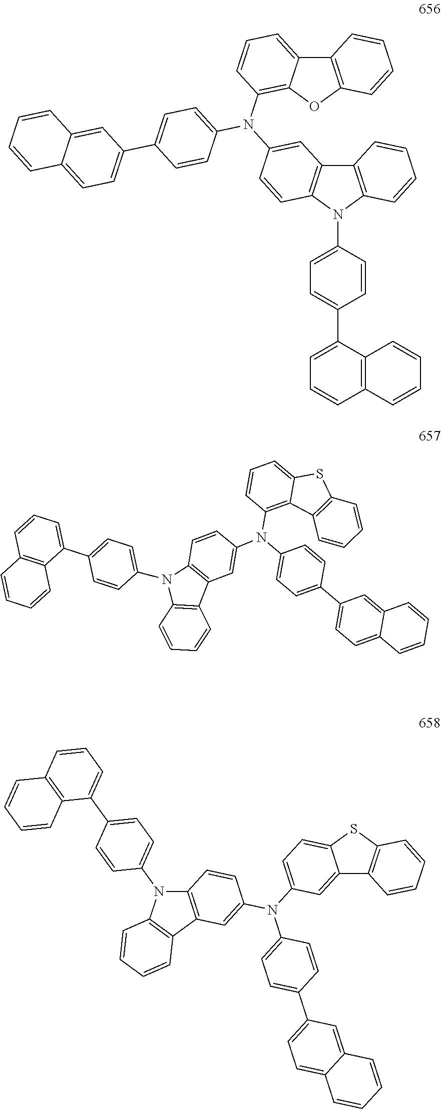 [SMILES: c1ccc2cc(-c3ccc(N(c4ccc5c(c4)c4ccccc4n5-c4ccc(-c5cccc6ccccc56)cc4)c4cccc5c4oc4ccccc45)cc3)ccc2c1 ; c1ccc2cc(-c3ccc(N(c4ccc5c(c4)c4ccccc4n5-c4ccc(-c5cccc6ccccc56)cc4)c4cccc5sc6ccccc6c45)cc3)ccc2c1 ; c1ccc2cc(-c3ccc(N(c4ccc5sc6ccccc6c5c4)c4ccc5c(c4)c4ccccc4n5-c4ccc(-c5cccc6ccccc56)cc4)cc3)ccc2c1]

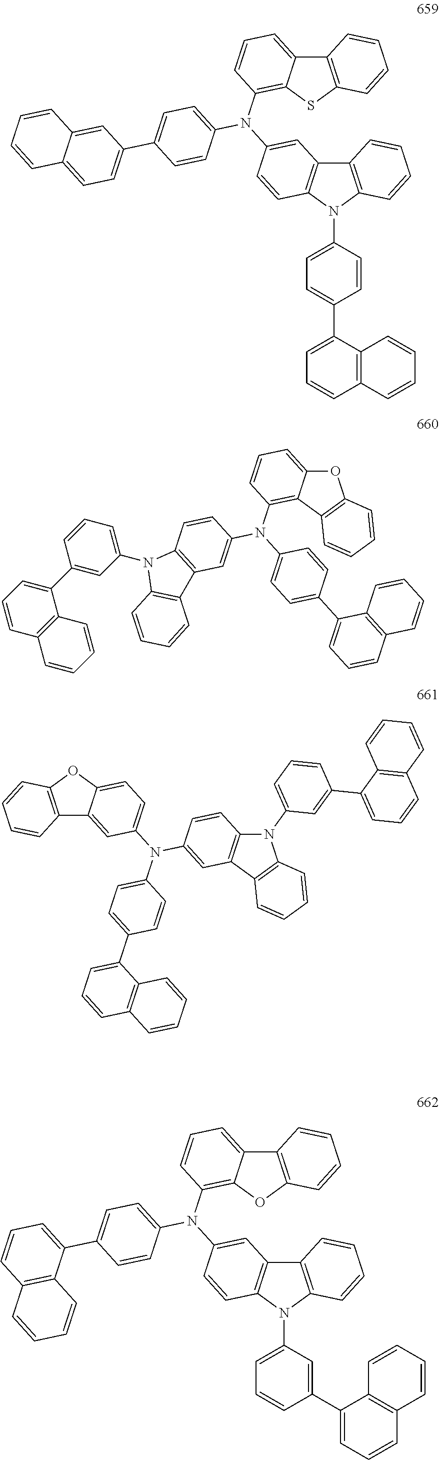 [SMILES: c1ccc2cc(-c3ccc(N(c4ccc5c(c4)c4ccccc4n5-c4ccc(-c5cccc6ccccc56)cc4)c4cccc5c4sc4ccccc45)cc3)ccc2c1 ; c1cc(-c2cccc3ccccc23)cc(-n2c3ccccc3c3cc(N(c4ccc(-c5cccc6ccccc56)cc4)c4cccc5oc6ccccc6c45)ccc32)c1 ; c1cc(-c2cccc3ccccc23)cc(-n2c3ccccc3c3cc(N(c4ccc(-c5cccc6ccccc56)cc4)c4ccc5oc6ccccc6c5c4)ccc32)c1 ; c1cc(-c2cccc3ccccc23)cc(-n2c3ccccc3c3cc(N(c4ccc(-c5cccc6ccccc56)cc4)c4cccc5c4oc4ccccc45)ccc32)c1]

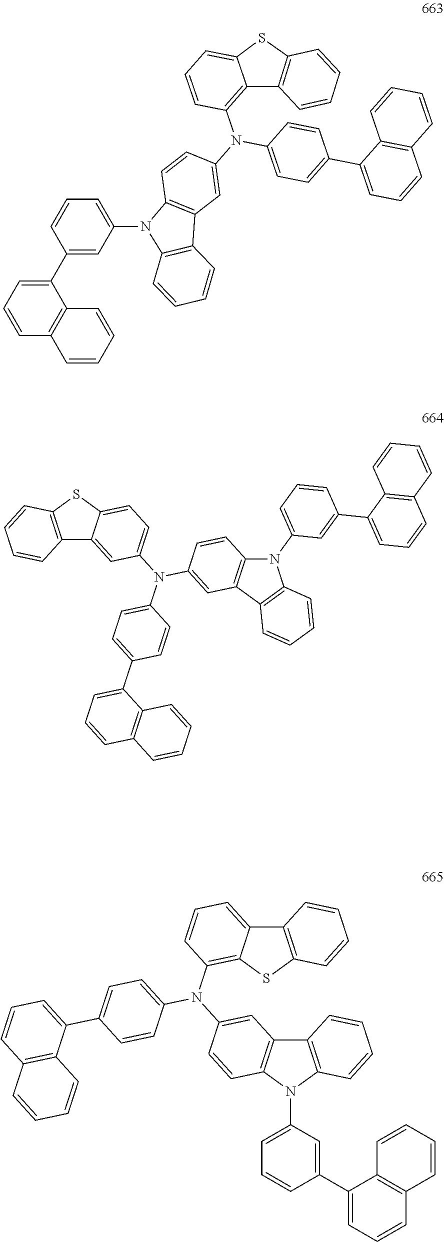 [SMILES: c1cc(-c2cccc3ccccc23)cc(-n2c3ccccc3c3cc(N(c4ccc(-c5cccc6ccccc56)cc4)c4cccc5sc6ccccc6c45)ccc32)c1 ; c1cc(-c2cccc3ccccc23)cc(-n2c3ccccc3c3cc(N(c4ccc(-c5cccc6ccccc56)cc4)c4ccc5sc6ccccc6c5c4)ccc32)c1 ; c1cc(-c2cccc3ccccc23)cc(-n2c3ccccc3c3cc(N(c4ccc(-c5cccc6ccccc56)cc4)c4cccc5c4sc4ccccc45)ccc32)c1]

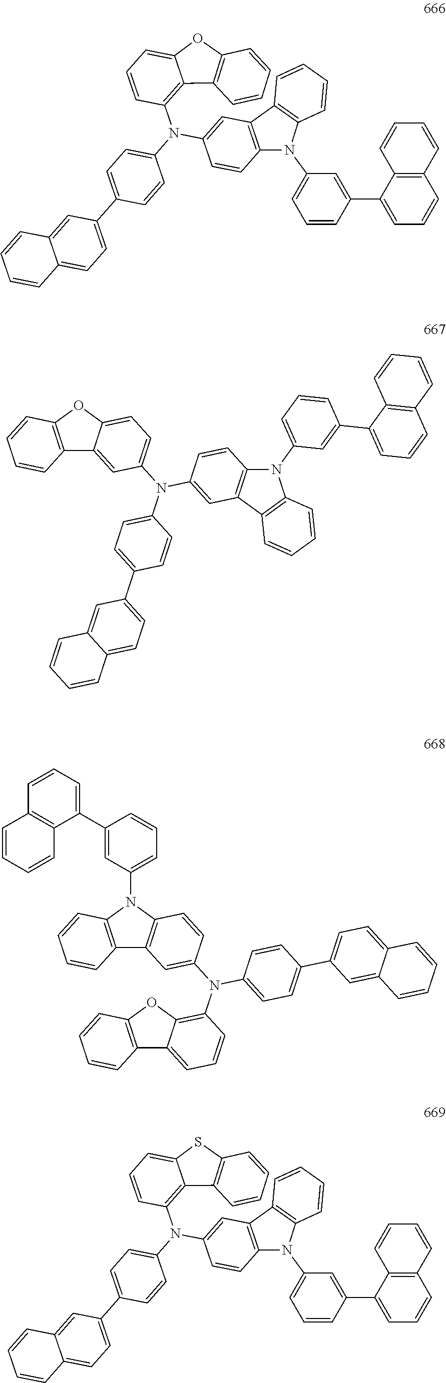 [SMILES: c1cc(-c2cccc3ccccc23)cc(-n2c3ccccc3c3cc(N(c4ccc(-c5ccc6ccccc6c5)cc4)c4cccc5oc6ccccc6c45)ccc32)c1 ; c1cc(-c2cccc3ccccc23)cc(-n2c3ccccc3c3cc(N(c4ccc(-c5ccc6ccccc6c5)cc4)c4ccc5oc6ccccc6c5c4)ccc32)c1 ; c1cc(-c2cccc3ccccc23)cc(-n2c3ccccc3c3cc(N(c4ccc(-c5ccc6ccccc6c5)cc4)c4cccc5c4oc4ccccc45)ccc32)c1 ; c1cc(-c2cccc3ccccc23)cc(-n2c3ccccc3c3cc(N(c4ccc(-c5ccc6ccccc6c5)cc4)c4cccc5sc6ccccc6c45)ccc32)c1]

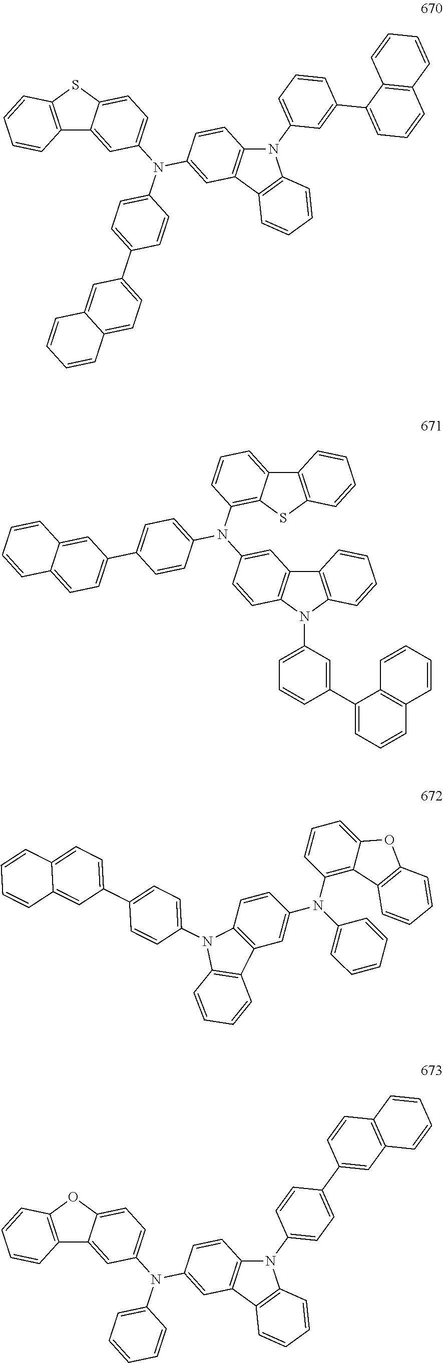 [SMILES: c1cc(-c2cccc3ccccc23)cc(-n2c3ccccc3c3cc(N(c4ccc(-c5ccc6ccccc6c5)cc4)c4ccc5sc6ccccc6c5c4)ccc32)c1 ; c1cc(-c2cccc3ccccc23)cc(-n2c3ccccc3c3cc(N(c4ccc(-c5ccc6ccccc6c5)cc4)c4cccc5c4sc4ccccc45)ccc32)c1 ; c1ccc(N(c2ccc3c(c2)c2ccccc2n3-c2ccc(-c3ccc4ccccc4c3)cc2)c2cccc3oc4ccccc4c23)cc1 ; c1ccc(N(c2ccc3oc4ccccc4c3c2)c2ccc3c(c2)c2ccccc2n3-c2ccc(-c3ccc4ccccc4c3)cc2)cc1]

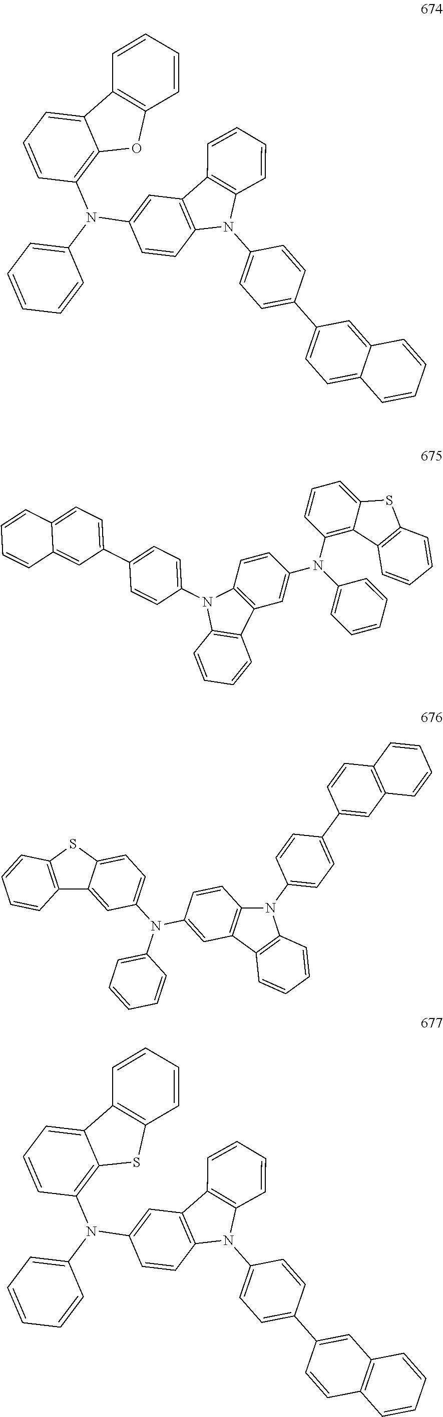

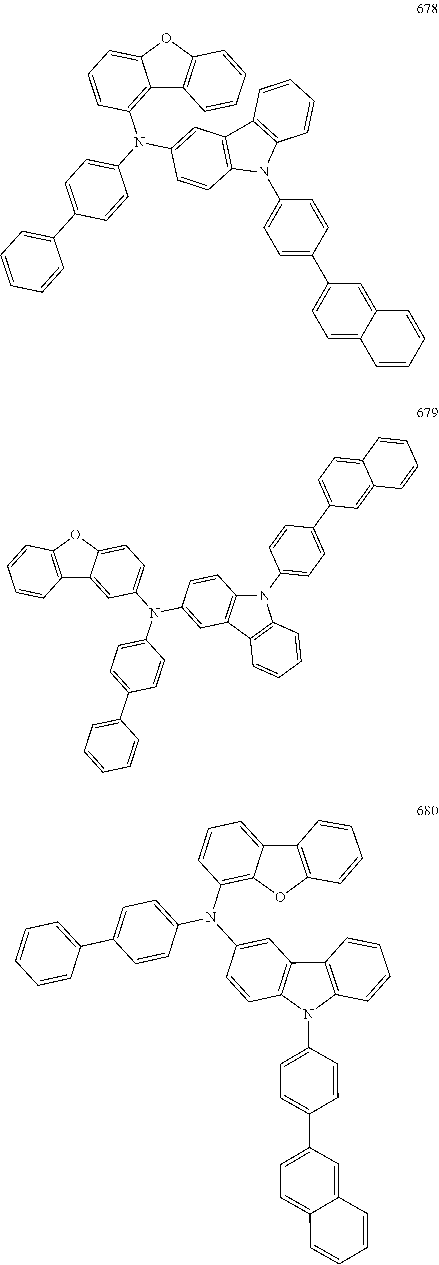 [SMILES: c1ccc(-c2ccc(N(c3ccc4c(c3)c3ccccc3n4-c3ccc(-c4ccc5ccccc5c4)cc3)c3cccc4oc5ccccc5c34)cc2)cc1 ; c1ccc(-c2ccc(N(c3ccc4oc5ccccc5c4c3)c3ccc4c(c3)c3ccccc3n4-c3ccc(-c4ccc5ccccc5c4)cc3)cc2)cc1 ; c1ccc(-c2ccc(N(c3ccc4c(c3)c3ccccc3n4-c3ccc(-c4ccc5ccccc5c4)cc3)c3cccc4c3oc3ccccc34)cc2)cc1]

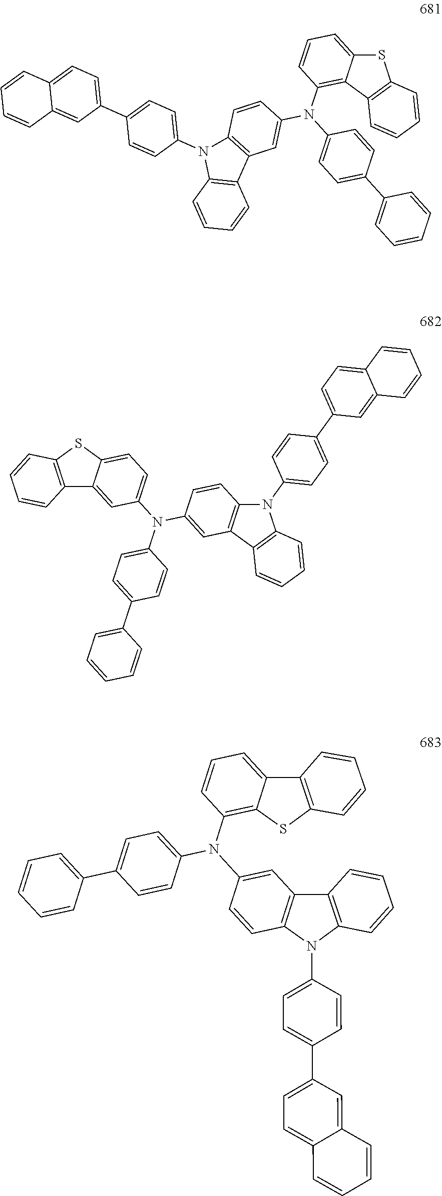 [SMILES: c1ccc(-c2ccc(N(c3ccc4c(c3)c3ccccc3n4-c3ccc(-c4ccc5ccccc5c4)cc3)c3cccc4sc5ccccc5c34)cc2)cc1 ; c1ccc(-c2ccc(N(c3ccc4sc5ccccc5c4c3)c3ccc4c(c3)c3ccccc3n4-c3ccc(-c4ccc5ccccc5c4)cc3)cc2)cc1 ; c1ccc(-c2ccc(N(c3ccc4c(c3)c3ccccc3n4-c3ccc(-c4ccc5ccccc5c4)cc3)c3cccc4c3sc3ccccc34)cc2)cc1]

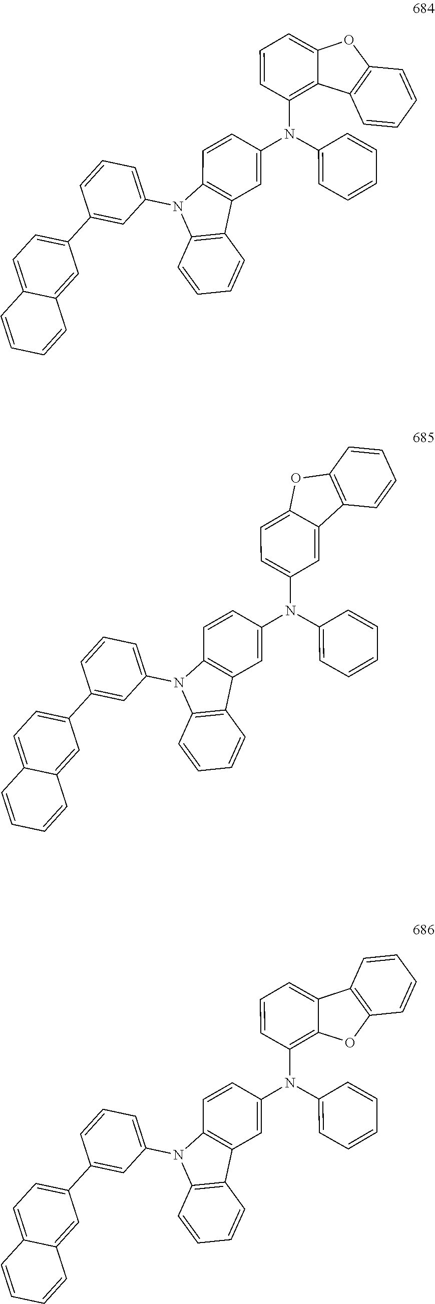 [SMILES: c1ccc(N(c2ccc3c(c2)c2ccccc2n3-c2cccc(-c3ccc4ccccc4c3)c2)c2cccc3oc4ccccc4c23)cc1 ; c1ccc(N(c2ccc3oc4ccccc4c3c2)c2ccc3c(c2)c2ccccc2n3-c2cccc(-c3ccc4ccccc4c3)c2)cc1 ; c1ccc(N(c2ccc3c(c2)c2ccccc2n3-c2cccc(-c3ccc4ccccc4c3)c2)c2cccc3c2oc2ccccc23)cc1]

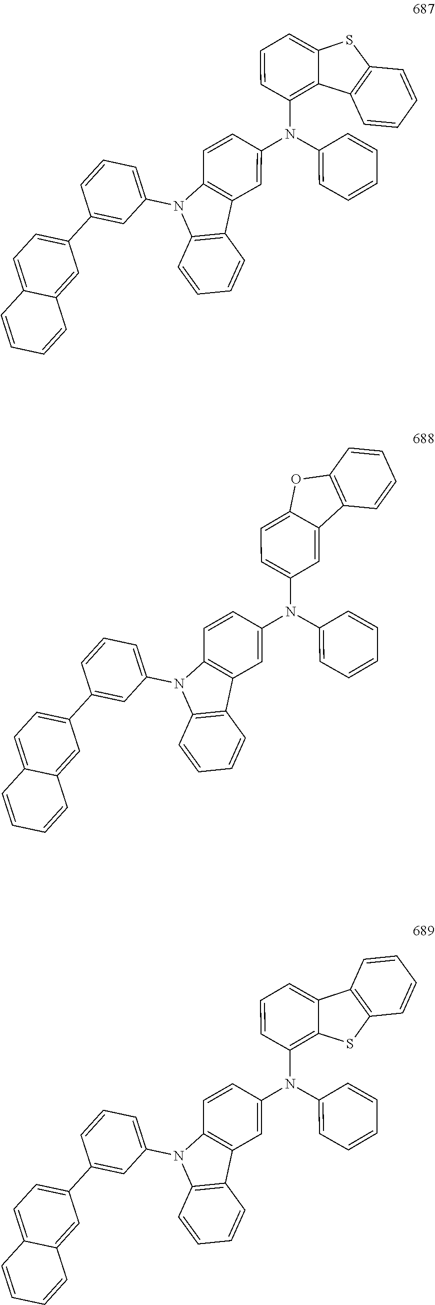 [SMILES: c1ccc(N(c2ccc3c(c2)c2ccccc2n3-c2cccc(-c3ccc4ccccc4c3)c2)c2cccc3sc4ccccc4c23)cc1 ; c1ccc(N(c2ccc3oc4ccccc4c3c2)c2ccc3c(c2)c2ccccc2n3-c2cccc(-c3ccc4ccccc4c3)c2)cc1 ; c1ccc(N(c2ccc3c(c2)c2ccccc2n3-c2cccc(-c3ccc4ccccc4c3)c2)c2cccc3c2sc2ccccc23)cc1]

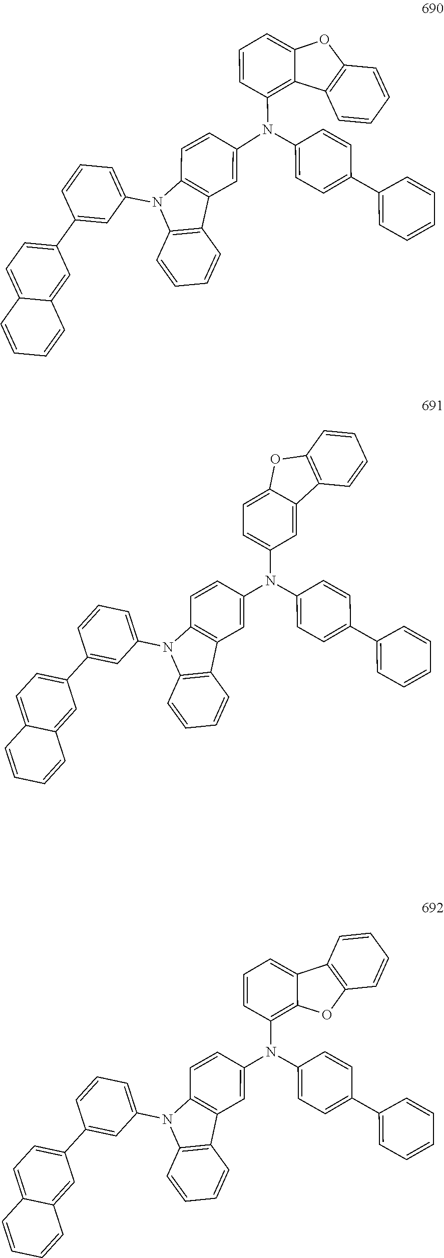 [SMILES: c1ccc(-c2ccc(N(c3ccc4c(c3)c3ccccc3n4-c3cccc(-c4ccc5ccccc5c4)c3)c3cccc4oc5ccccc5c34)cc2)cc1 ; c1ccc(-c2ccc(N(c3ccc4oc5ccccc5c4c3)c3ccc4c(c3)c3ccccc3n4-c3cccc(-c4ccc5ccccc5c4)c3)cc2)cc1 ; c1ccc(-c2ccc(N(c3ccc4c(c3)c3ccccc3n4-c3cccc(-c4ccc5ccccc5c4)c3)c3cccc4c3oc3ccccc34)cc2)cc1]

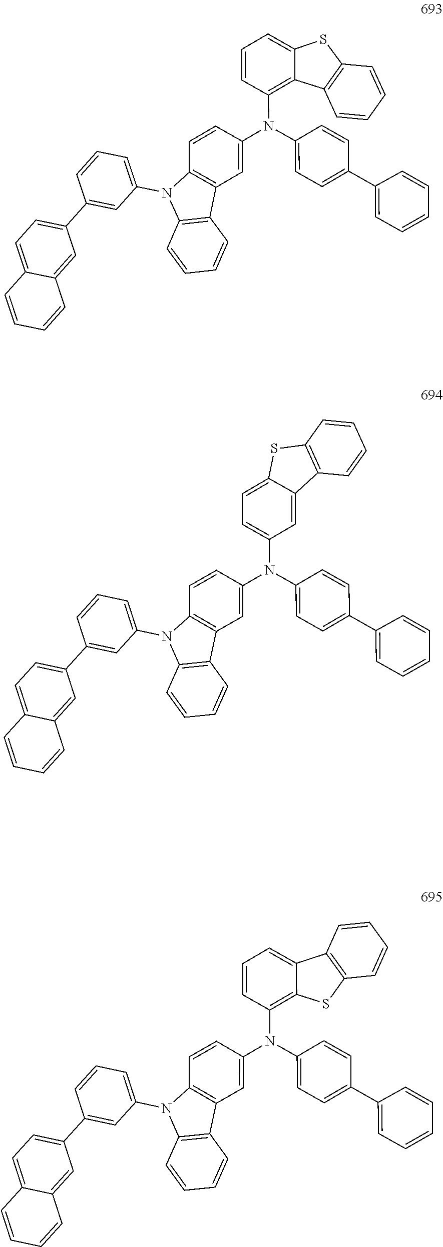 [SMILES: c1ccc(-c2ccc(N(c3ccc4c(c3)c3ccccc3n4-c3cccc(-c4ccc5ccccc5c4)c3)c3cccc4sc5ccccc5c34)cc2)cc1 ; c1ccc(-c2ccc(N(c3ccc4sc5ccccc5c4c3)c3ccc4c(c3)c3ccccc3n4-c3cccc(-c4ccc5ccccc5c4)c3)cc2)cc1 ; c1ccc(-c2ccc(N(c3ccc4c(c3)c3ccccc3n4-c3cccc(-c4ccc5ccccc5c4)c3)c3cccc4c3sc3ccccc34)cc2)cc1]

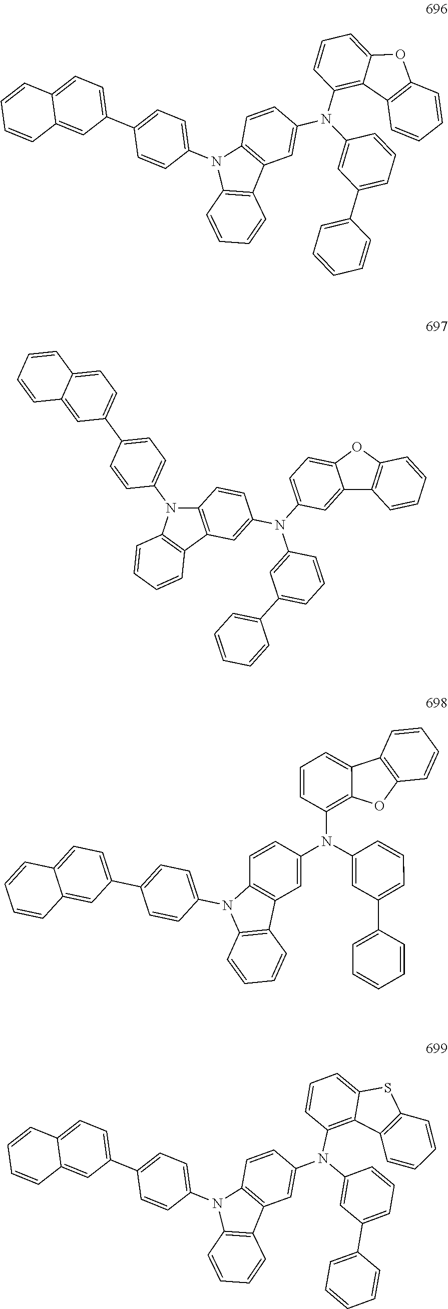 [SMILES: c1ccc(-c2cccc(N(c3ccc4c(c3)c3ccccc3n4-c3ccc(-c4ccc5ccccc5c4)cc3)c3cccc4oc5ccccc5c34)c2)cc1 ; c1ccc(-c2cccc(N(c3ccc4oc5ccccc5c4c3)c3ccc4c(c3)c3ccccc3n4-c3ccc(-c4ccc5ccccc5c4)cc3)c2)cc1 ; c1ccc(-c2cccc(N(c3ccc4c(c3)c3ccccc3n4-c3ccc(-c4ccc5ccccc5c4)cc3)c3cccc4c3oc3ccccc34)c2)cc1 ; c1ccc(-c2cccc(N(c3ccc4c(c3)c3ccccc3n4-c3ccc(-c4ccc5ccccc5c4)cc3)c3cccc4sc5ccccc5c34)c2)cc1]

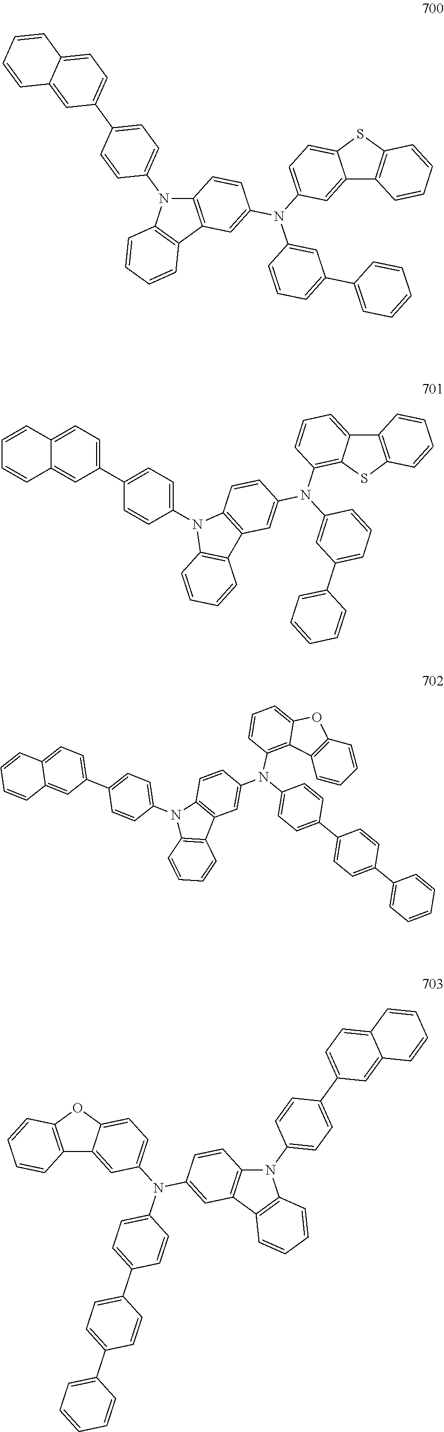 [SMILES: c1ccc(-c2cccc(N(c3ccc4sc5ccccc5c4c3)c3ccc4c(c3)c3ccccc3n4-c3ccc(-c4ccc5ccccc5c4)cc3)c2)cc1 ; c1ccc(-c2cccc(N(c3ccc4c(c3)c3ccccc3n4-c3ccc(-c4ccc5ccccc5c4)cc3)c3cccc4c3sc3ccccc34)c2)cc1 ; c1ccc(-c2ccc(-c3ccc(N(c4ccc5c(c4)c4ccccc4n5-c4ccc(-c5ccc6ccccc6c5)cc4)c4cccc5oc6ccccc6c45)cc3)cc2)cc1 ; c1ccc(-c2ccc(-c3ccc(N(c4ccc5oc6ccccc6c5c4)c4ccc5c(c4)c4ccccc4n5-c4ccc(-c5ccc6ccccc6c5)cc4)cc3)cc2)cc1]

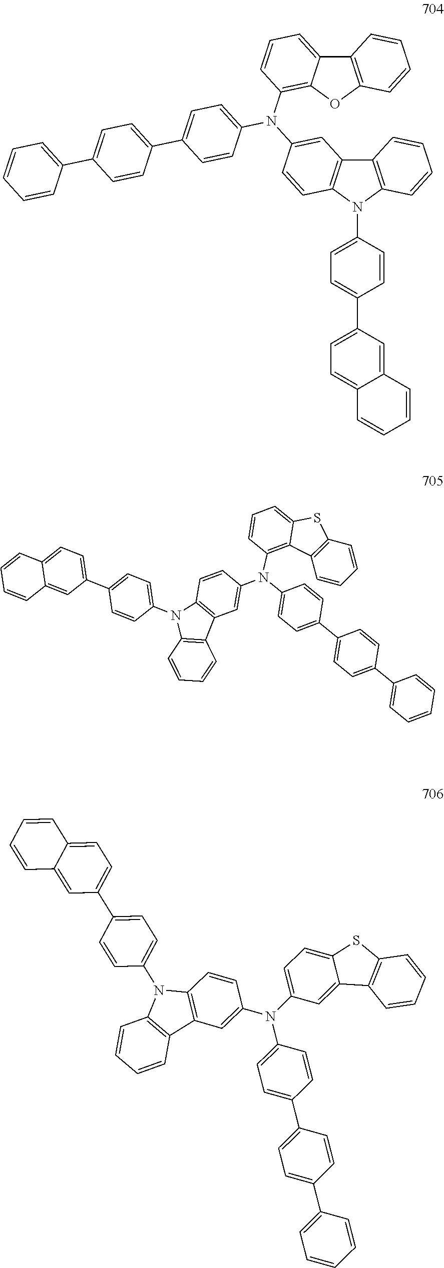 [SMILES: c1ccc(-c2ccc(-c3ccc(N(c4ccc5c(c4)c4ccccc4n5-c4ccc(-c5ccc6ccccc6c5)cc4)c4cccc5c4oc4ccccc45)cc3)cc2)cc1 ; c1ccc(-c2ccc(-c3ccc(N(c4ccc5c(c4)c4ccccc4n5-c4ccc(-c5ccc6ccccc6c5)cc4)c4cccc5sc6ccccc6c45)cc3)cc2)cc1 ; c1ccc(-c2ccc(-c3ccc(N(c4ccc5sc6ccccc6c5c4)c4ccc5c(c4)c4ccccc4n5-c4ccc(-c5ccc6ccccc6c5)cc4)cc3)cc2)cc1]

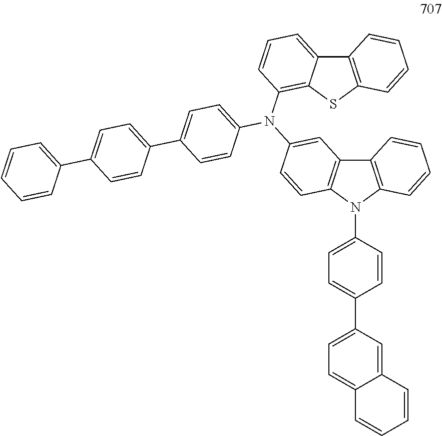 [SMILES: c1ccc(-c2ccc(-c3ccc(N(c4ccc5c(c4)c4ccccc4n5-c4ccc(-c5ccc6ccccc6c5)cc4)c4cccc5c4sc4ccccc45)cc3)cc2)cc1]

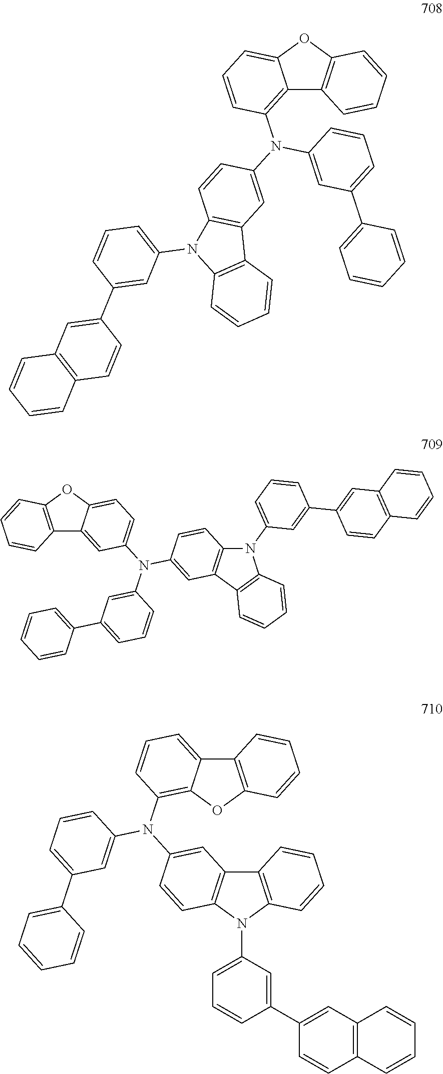 [SMILES: c1ccc(-c2cccc(N(c3ccc4c(c3)c3ccccc3n4-c3cccc(-c4ccc5ccccc5c4)c3)c3cccc4oc5ccccc5c34)c2)cc1 ; c1ccc(-c2cccc(N(c3ccc4oc5ccccc5c4c3)c3ccc4c(c3)c3ccccc3n4-c3cccc(-c4ccc5ccccc5c4)c3)c2)cc1 ; c1ccc(-c2cccc(N(c3ccc4c(c3)c3ccccc3n4-c3cccc(-c4ccc5ccccc5c4)c3)c3cccc4c3oc3ccccc34)c2)cc1]

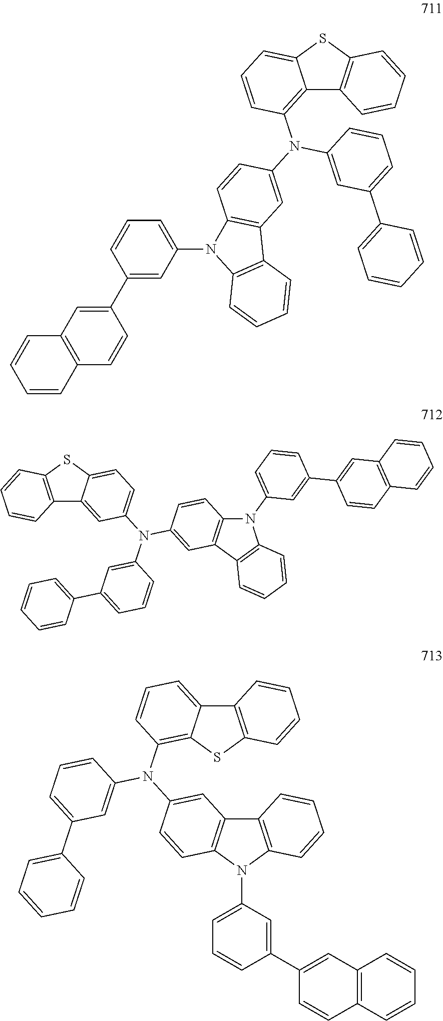 [SMILES: c1ccc(-c2cccc(N(c3ccc4c(c3)c3ccccc3n4-c3cccc(-c4ccc5ccccc5c4)c3)c3cccc4sc5ccccc5c34)c2)cc1 ; c1ccc(-c2cccc(N(c3ccc4sc5ccccc5c4c3)c3ccc4c(c3)c3ccccc3n4-c3cccc(-c4ccc5ccccc5c4)c3)c2)cc1 ; c1ccc(-c2cccc(N(c3ccc4c(c3)c3ccccc3n4-c3cccc(-c4ccc5ccccc5c4)c3)c3cccc4c3sc3ccccc34)c2)cc1]

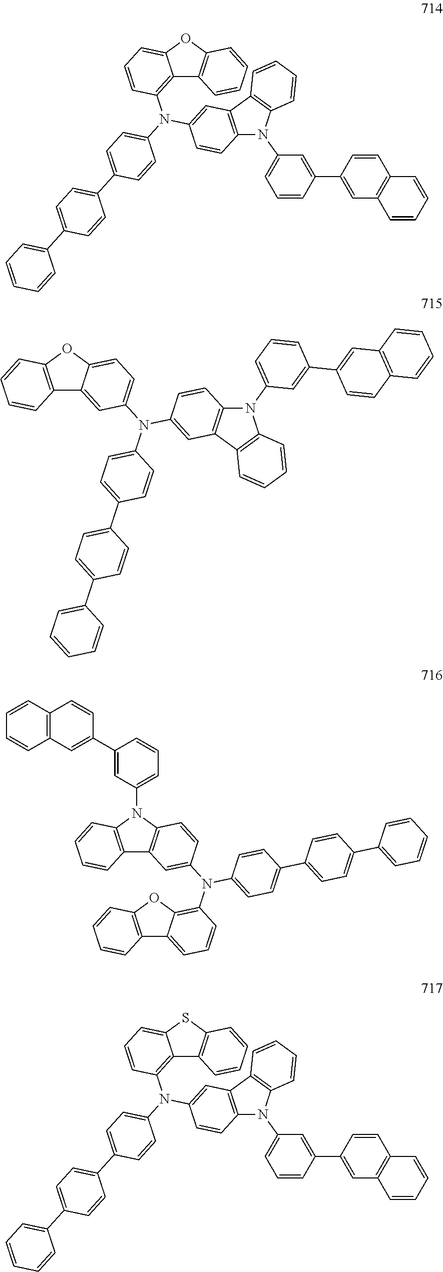 [SMILES: c1ccc(-c2ccc(-c3ccc(N(c4ccc5c(c4)c4ccccc4n5-c4cccc(-c5ccc6ccccc6c5)c4)c4cccc5oc6ccccc6c45)cc3)cc2)cc1 ; c1ccc(-c2ccc(-c3ccc(N(c4ccc5oc6ccccc6c5c4)c4ccc5c(c4)c4ccccc4n5-c4cccc(-c5ccc6ccccc6c5)c4)cc3)cc2)cc1 ; c1ccc(-c2ccc(-c3ccc(N(c4ccc5c(c4)c4ccccc4n5-c4cccc(-c5ccc6ccccc6c5)c4)c4cccc5c4oc4ccccc45)cc3)cc2)cc1 ; c1ccc(-c2ccc(-c3ccc(N(c4ccc5c(c4)c4ccccc4n5-c4cccc(-c5ccc6ccccc6c5)c4)c4cccc5sc6ccccc6c45)cc3)cc2)cc1]

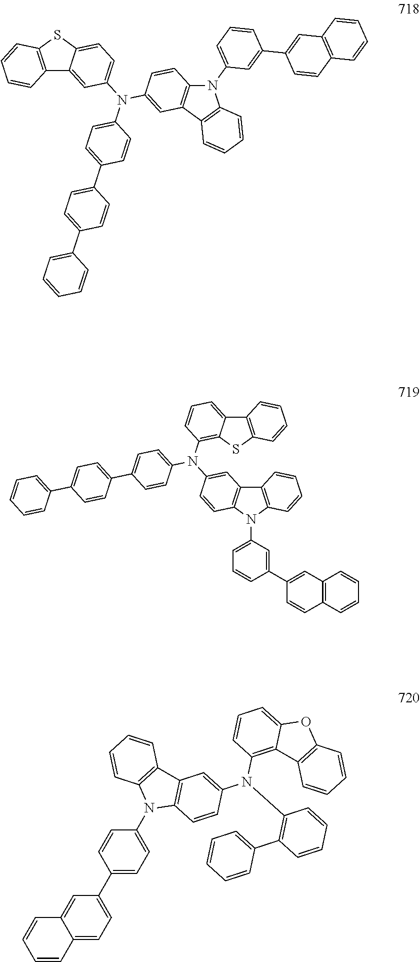 [SMILES: c1ccc(-c2ccc(-c3ccc(N(c4ccc5sc6ccccc6c5c4)c4ccc5c(c4)c4ccccc4n5-c4cccc(-c5ccc6ccccc6c5)c4)cc3)cc2)cc1 ; c1ccc(-c2ccc(-c3ccc(N(c4ccc5c(c4)c4ccccc4n5-c4cccc(-c5ccc6ccccc6c5)c4)c4cccc5c4sc4ccccc45)cc3)cc2)cc1 ; c1ccc(-c2ccccc2CN(c2ccc3c(c2)c2ccccc2n3-c2ccc(-c3ccc4ccccc4c3)cc2)c2cccc3oc4ccccc4c23)cc1]

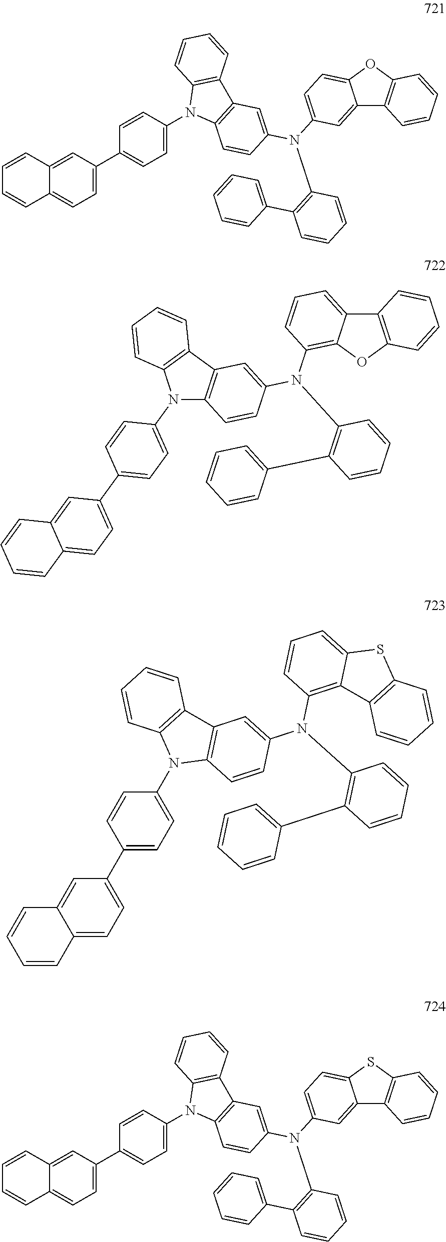 [SMILES: c1ccc(-c2ccccc2CN(c2ccc3oc4ccccc4c3c2)c2ccc3c(c2)c2ccccc2n3-c2ccc(-c3ccc4ccccc4c3)cc2)cc1 ; c1ccc(Cc2ccccc2CN(c2ccc3c(c2)c2ccccc2n3-c2ccc(-c3ccc4ccccc4c3)cc2)c2cccc3c2oc2ccccc23)cc1 ; c1ccc(Cc2ccccc2CN(c2ccc3c(c2)c2ccccc2n3-c2ccc(-c3ccc4ccccc4c3)cc2)c2cccc3sc4ccccc4c23)cc1 ; c1ccc(-c2ccccc2CN(c2ccc3sc4ccccc4c3c2)c2ccc3c(c2)c2ccccc2n3-c2ccc(-c3ccc4ccccc4c3)cc2)cc1]

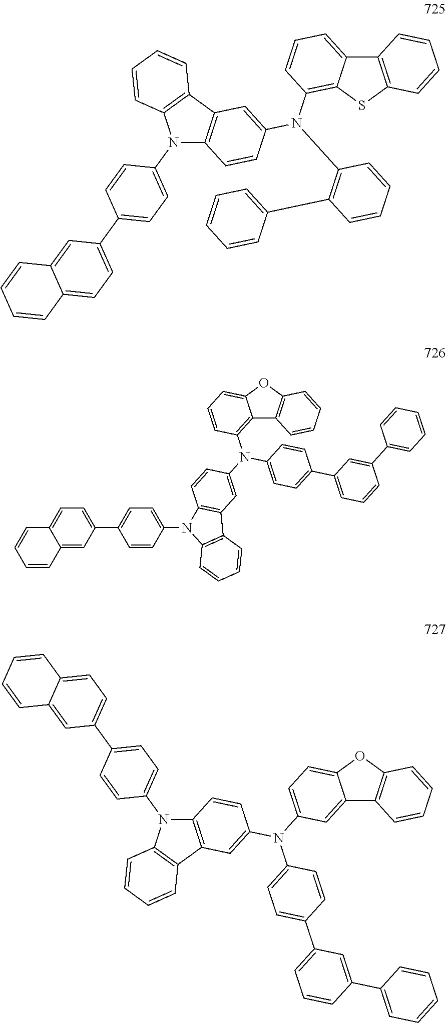 [SMILES: c1ccc(Cc2ccccc2CN(c2ccc3c(c2)c2ccccc2n3-c2ccc(-c3ccc4ccccc4c3)cc2)c2cccc3c2sc2ccccc23)cc1 ; c1ccc(-c2cccc(-c3ccc(N(c4ccc5c(c4)c4ccccc4n5-c4ccc(-c5ccc6ccccc6c5)cc4)c4cccc5oc6ccccc6c45)cc3)c2)cc1 ; c1ccc(-c2cccc(-c3ccc(N(c4ccc5oc6ccccc6c5c4)c4ccc5c(c4)c4ccccc4n5-c4ccc(-c5ccc6ccccc6c5)cc4)cc3)c2)cc1]

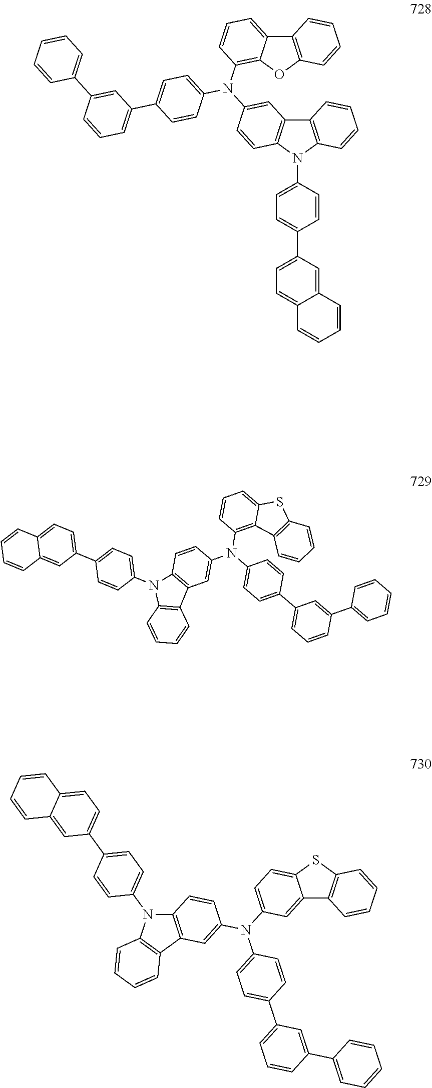 [SMILES: c1ccc(-c2cccc(-c3ccc(N(c4ccc5c(c4)c4ccccc4n5-c4ccc(-c5ccc6ccccc6c5)cc4)c4cccc5c4oc4ccccc45)cc3)c2)cc1 ; c1ccc(-c2cccc(-c3ccc(N(c4ccc5c(c4)c4ccccc4n5-c4ccc(-c5ccc6ccccc6c5)cc4)c4cccc5sc6ccccc6c45)cc3)c2)cc1 ; c1ccc(-c2cccc(-c3ccc(N(c4ccc5sc6ccccc6c5c4)c4ccc5c(c4)c4ccccc4n5-c4ccc(-c5ccc6ccccc6c5)cc4)cc3)c2)cc1]

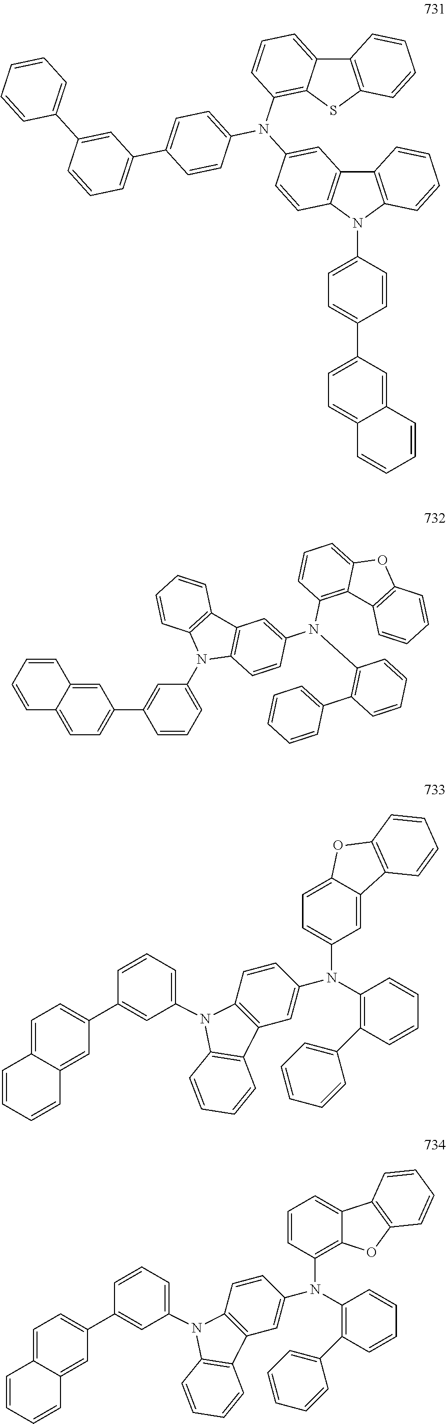 [SMILES: c1ccc(-c2cccc(-c3ccc(N(c4ccc5c(c4)c4ccccc4n5-c4ccc(-c5ccc6ccccc6c5)cc4)c4cccc5c4sc4ccccc45)cc3)c2)cc1 ; c1ccc(-c2ccccc2CN(c2ccc3c(c2)c2ccccc2n3-c2cccc(-c3ccc4ccccc4c3)c2)c2cccc3oc4ccccc4c23)cc1 ; c1ccc(-c2ccccc2N(c2ccc3oc4ccccc4c3c2)c2ccc3c(c2)c2ccccc2n3-c2cccc(-c3ccc4ccccc4c3)c2)cc1 ; c1ccc(-c2ccccc2N(c2ccc3c(c2)c2ccccc2n3-c2cccc(-c3ccc4ccccc4c3)c2)c2cccc3c2oc2ccccc23)cc1]

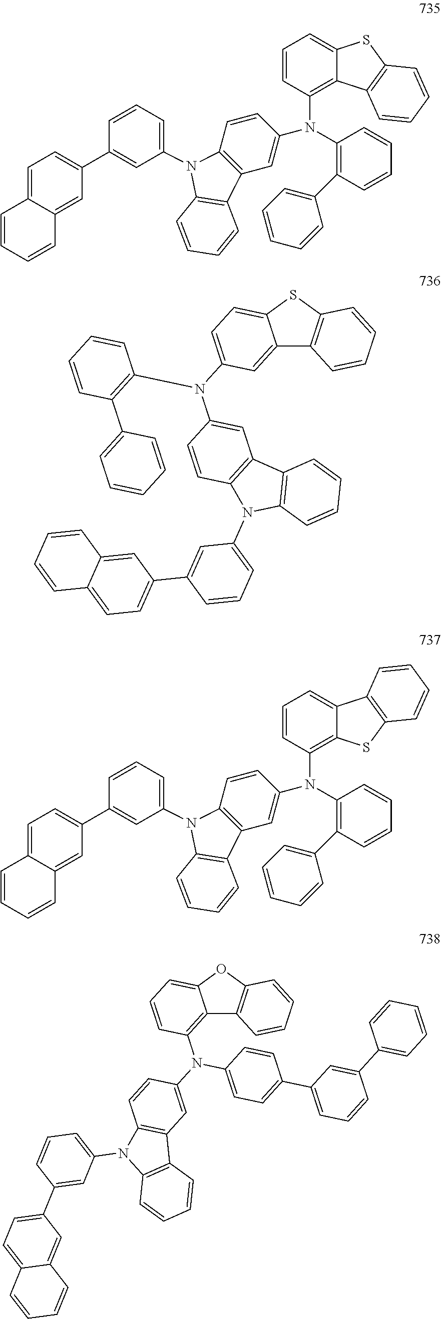 [SMILES: c1ccc(-c2ccccc2N(c2ccc3c(c2)c2ccccc2n3-c2cccc(-c3ccc4ccccc4c3)c2)c2cccc3sc4ccccc4c23)cc1 ; c1ccc(-c2ccccc2CN(c2ccc3sc4ccccc4c3c2)c2ccc3c(c2)c2ccccc2n3-c2cccc(-c3ccc4ccccc4c3)c2)cc1 ; c1ccc(-c2ccccc2N(c2ccc3c(c2)c2ccccc2n3-c2cccc(-c3ccc4ccccc4c3)c2)c2cccc3c2sc2ccccc23)cc1 ; c1ccc(-c2cccc(-c3ccc(N(c4ccc5c(c4)c4ccccc4n5-c4cccc(-c5ccc6ccccc6c5)c4)c4cccc5oc6ccccc6c45)cc3)c2)cc1]

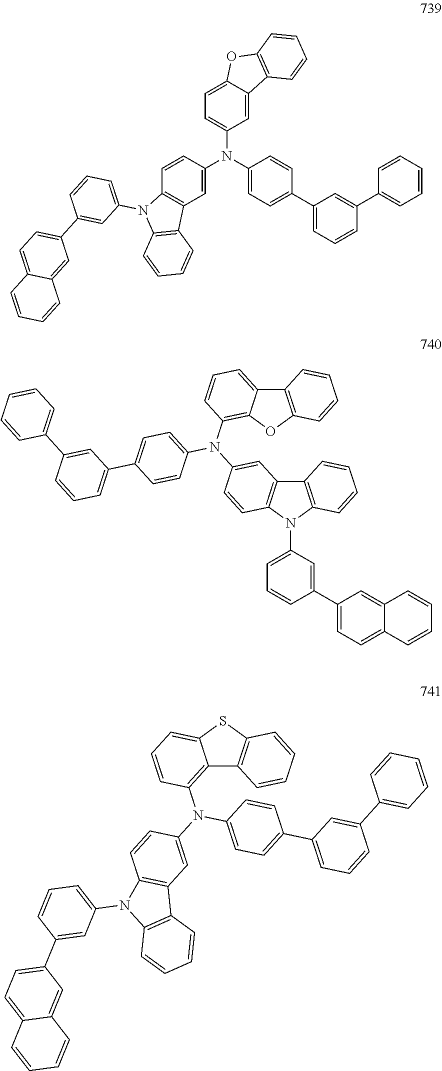 [SMILES: c1ccc(-c2cccc(-c3ccc(N(c4ccc5oc6ccccc6c5c4)c4ccc5c(c4)c4ccccc4n5-c4cccc(-c5ccc6ccccc6c5)c4)cc3)c2)cc1 ; c1ccc(-c2cccc(-c3ccc(N(c4ccc5c(c4)c4ccccc4n5-c4cccc(-c5ccc6ccccc6c5)c4)c4cccc5c4oc4ccccc45)cc3)c2)cc1 ; c1ccc(-c2cccc(-c3ccc(N(c4ccc5c(c4)c4ccccc4n5-c4cccc(-c5ccc6ccccc6c5)c4)c4cccc5sc6ccccc6c45)cc3)c2)cc1]

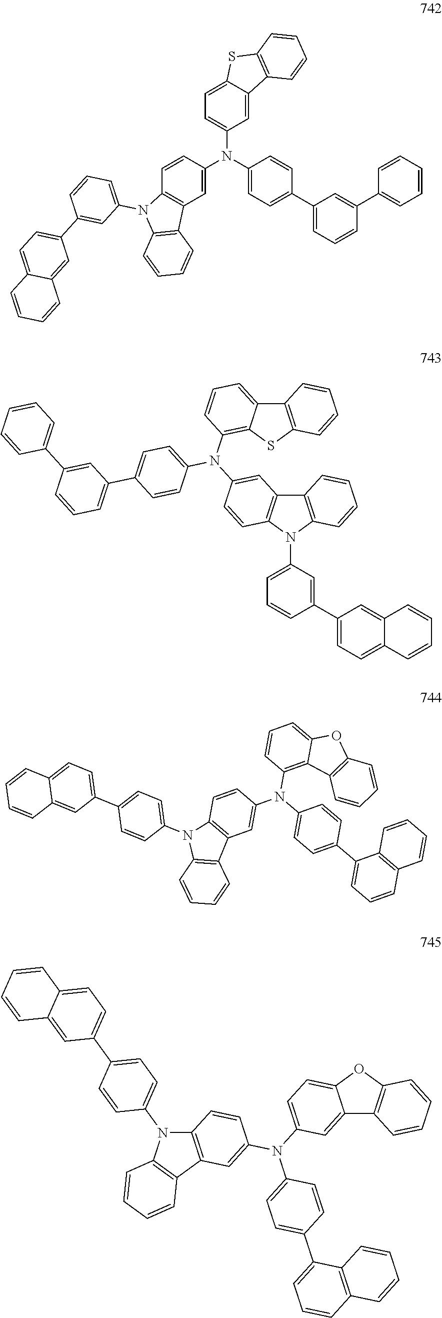 [SMILES: c1ccc(-c2cccc(-c3ccc(N(c4ccc5sc6ccccc6c5c4)c4ccc5c(c4)c4ccccc4n5-c4cccc(-c5ccc6ccccc6c5)c4)cc3)c2)cc1 ; c1ccc(-c2cccc(-c3ccc(N(c4ccc5c(c4)c4ccccc4n5-c4cccc(-c5ccc6ccccc6c5)c4)c4cccc5c4sc4ccccc45)cc3)c2)cc1 ; c1ccc2cc(-c3ccc(-n4c5ccccc5c5cc(N(c6ccc(-c7cccc8ccccc78)cc6)c6cccc7oc8ccccc8c67)ccc54)cc3)ccc2c1 ; c1ccc2cc(-c3ccc(-n4c5ccccc5c5cc(N(c6ccc(-c7cccc8ccccc78)cc6)c6ccc7oc8ccccc8c7c6)ccc54)cc3)ccc2c1]

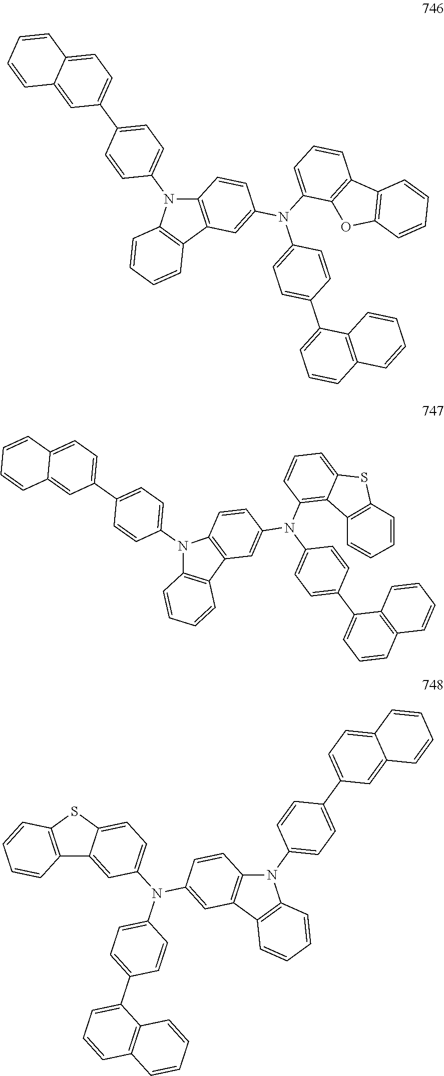 [SMILES: c1ccc2cc(-c3ccc(-n4c5ccccc5c5cc(N(c6ccc(-c7cccc8ccccc78)cc6)c6cccc7c6oc6ccccc67)ccc54)cc3)ccc2c1 ; c1ccc2cc(-c3ccc(-n4c5ccccc5c5cc(N(c6ccc(-c7cccc8ccccc78)cc6)c6cccc7sc8ccccc8c67)ccc54)cc3)ccc2c1 ; c1ccc2cc(-c3ccc(-n4c5ccccc5c5cc(N(c6ccc(-c7cccc8ccccc78)cc6)c6ccc7sc8ccccc8c7c6)ccc54)cc3)ccc2c1]

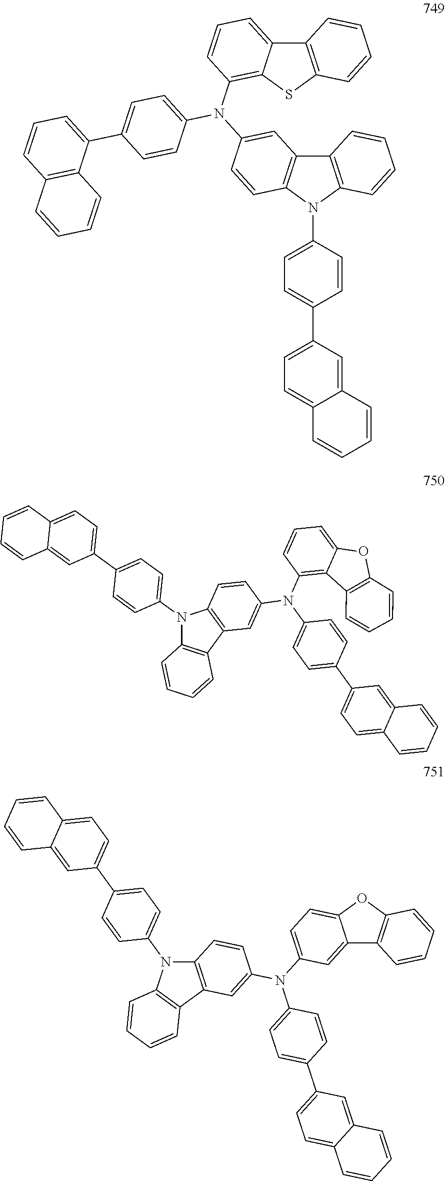 [SMILES: c1ccc2cc(-c3ccc(-n4c5ccccc5c5cc(N(c6ccc(-c7cccc8ccccc78)cc6)c6cccc7c6sc6ccccc67)ccc54)cc3)ccc2c1 ; c1ccc2cc(-c3ccc(N(c4ccc5c(c4)c4ccccc4n5-c4ccc(-c5ccc6ccccc6c5)cc4)c4cccc5oc6ccccc6c45)cc3)ccc2c1 ; c1ccc2cc(-c3ccc(N(c4ccc5oc6ccccc6c5c4)c4ccc5c(c4)c4ccccc4n5-c4ccc(-c5ccc6ccccc6c5)cc4)cc3)ccc2c1]

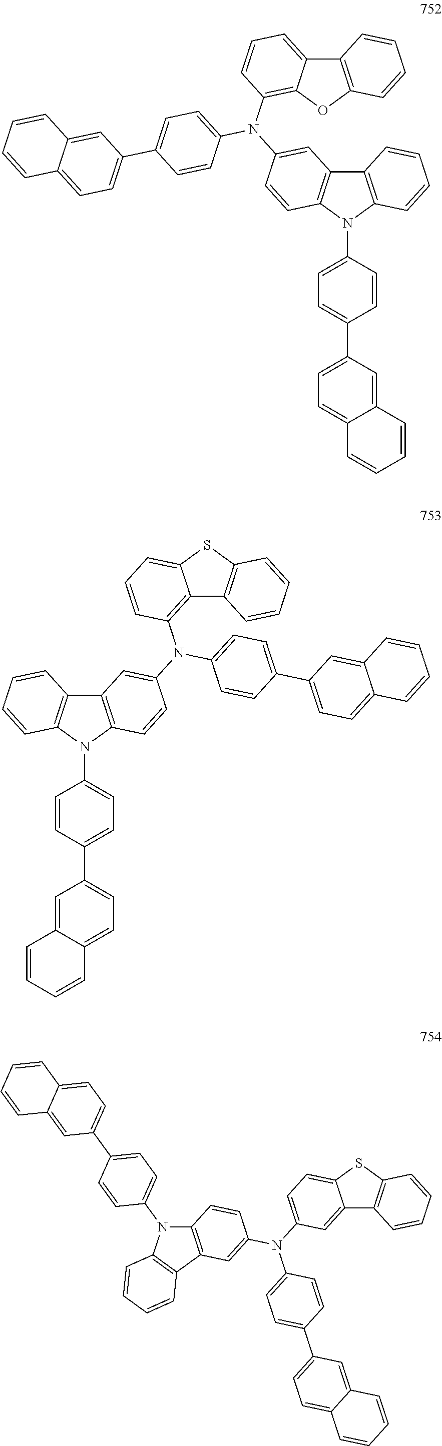 [SMILES: c1ccc2cc(-c3ccc(N(c4ccc5c(c4)c4ccccc4n5-c4ccc(-c5ccc6ccccc6c5)cc4)c4cccc5c4oc4ccccc45)cc3)ccc2c1 ; c1ccc2cc(-c3ccc(N(c4ccc5c(c4)c4ccccc4n5-c4ccc(-c5ccc6ccccc6c5)cc4)c4cccc5sc6ccccc6c45)cc3)ccc2c1 ; c1ccc2cc(-c3ccc(N(c4ccc5sc6ccccc6c5c4)c4ccc5c(c4)c4ccccc4n5-c4ccc(-c5ccc6ccccc6c5)cc4)cc3)ccc2c1]

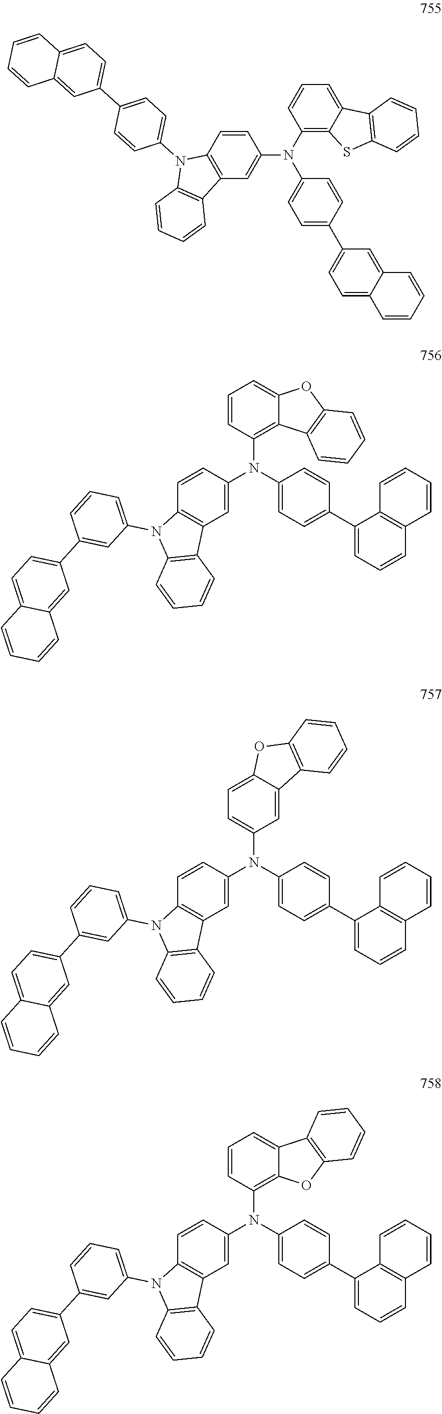 [SMILES: c1ccc2cc(-c3ccc(N(c4ccc5c(c4)c4ccccc4n5-c4ccc(-c5ccc6ccccc6c5)cc4)c4cccc5c4sc4ccccc45)cc3)ccc2c1 ; c1cc(-c2ccc3ccccc3c2)cc(-n2c3ccccc3c3cc(N(c4ccc(-c5cccc6ccccc56)cc4)c4cccc5oc6ccccc6c45)ccc32)c1 ; c1cc(-c2ccc3ccccc3c2)cc(-n2c3ccccc3c3cc(N(c4ccc(-c5cccc6ccccc56)cc4)c4ccc5oc6ccccc6c5c4)ccc32)c1 ; c1cc(-c2ccc3ccccc3c2)cc(-n2c3ccccc3c3cc(N(c4ccc(-c5cccc6ccccc56)cc4)c4cccc5c4oc4ccccc45)ccc32)c1]

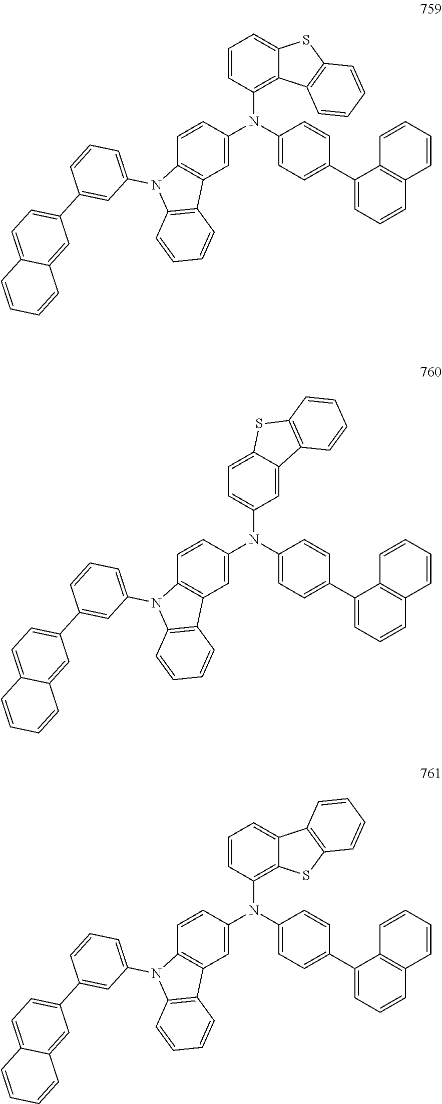 [SMILES: c1cc(-c2ccc3ccccc3c2)cc(-n2c3ccccc3c3cc(N(c4ccc(-c5cccc6ccccc56)cc4)c4cccc5sc6ccccc6c45)ccc32)c1 ; c1cc(-c2ccc3ccccc3c2)cc(-n2c3ccccc3c3cc(N(c4ccc(-c5cccc6ccccc56)cc4)c4ccc5sc6ccccc6c5c4)ccc32)c1 ; c1cc(-c2ccc3ccccc3c2)cc(-n2c3ccccc3c3cc(N(c4ccc(-c5cccc6ccccc56)cc4)c4cccc5c4sc4ccccc45)ccc32)c1]

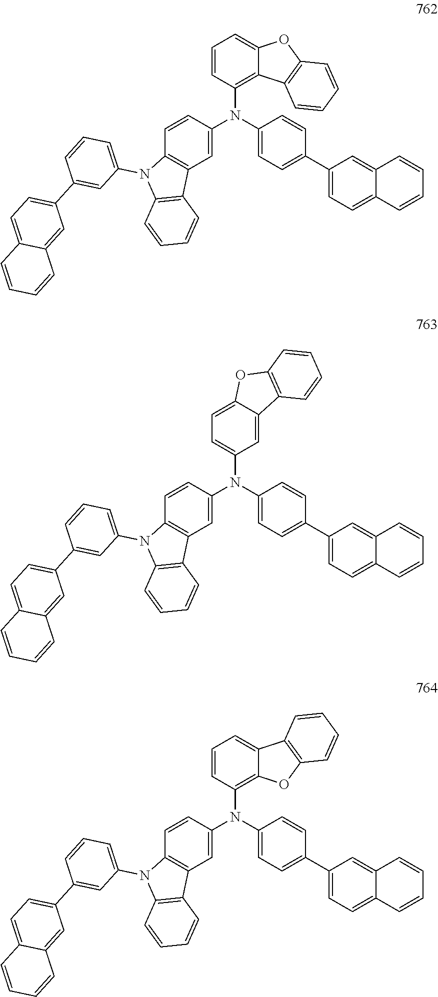 [SMILES: c1cc(-c2ccc3ccccc3c2)cc(-n2c3ccccc3c3cc(N(c4ccc(-c5ccc6ccccc6c5)cc4)c4cccc5oc6ccccc6c45)ccc32)c1 ; c1cc(-c2ccc3ccccc3c2)cc(-n2c3ccccc3c3cc(N(c4ccc(-c5ccc6ccccc6c5)cc4)c4ccc5oc6ccccc6c5c4)ccc32)c1 ; c1cc(-c2ccc3ccccc3c2)cc(-n2c3ccccc3c3cc(N(c4ccc(-c5ccc6ccccc6c5)cc4)c4cccc5c4oc4ccccc45)ccc32)c1]

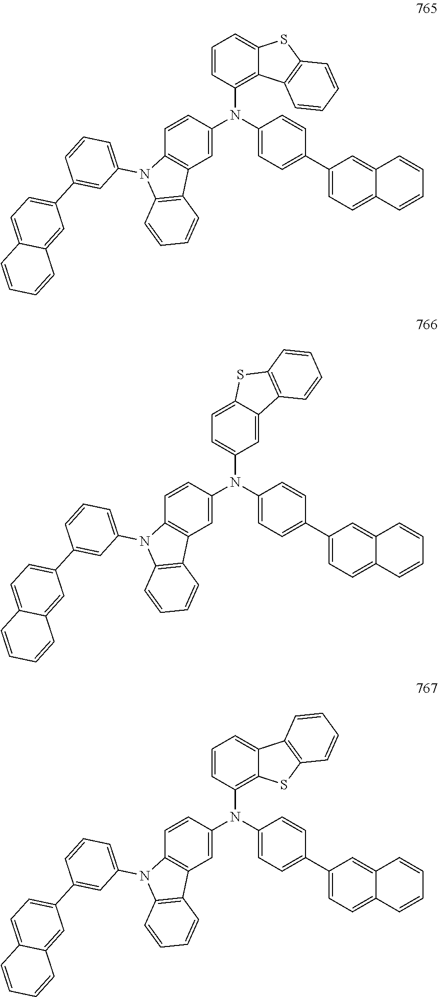 [SMILES: c1cc(-c2ccc3ccccc3c2)cc(-n2c3ccccc3c3cc(N(c4ccc(-c5ccc6ccccc6c5)cc4)c4cccc5sc6ccccc6c45)ccc32)c1 ; c1cc(-c2ccc3ccccc3c2)cc(-n2c3ccccc3c3cc(N(c4ccc(-c5ccc6ccccc6c5)cc4)c4ccc5sc6ccccc6c5c4)ccc32)c1 ; c1cc(-c2ccc3ccccc3c2)cc(-n2c3ccccc3c3cc(N(c4ccc(-c5ccc6ccccc6c5)cc4)c4cccc5c4sc4ccccc45)ccc32)c1]

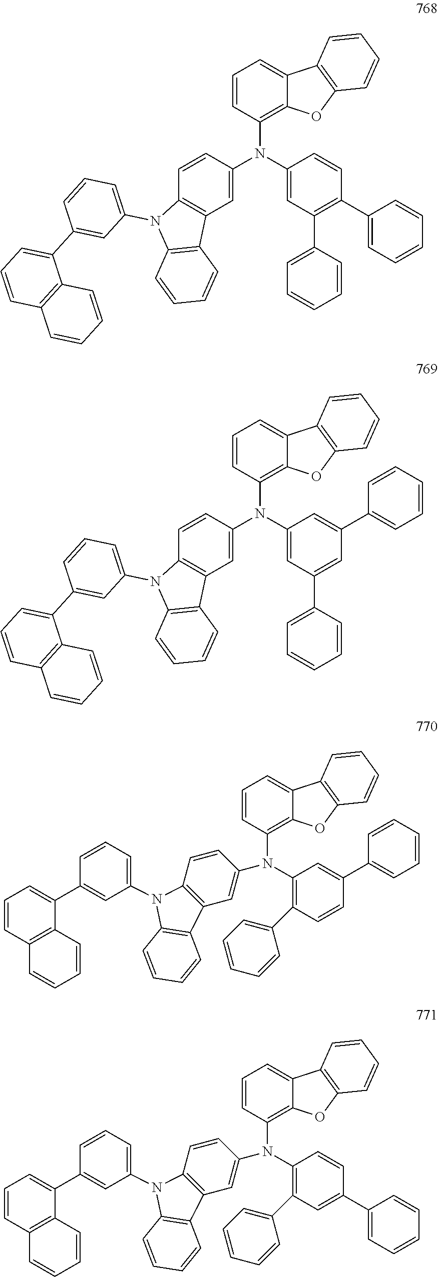 [SMILES: c1ccc(-c2ccc(N(c3ccc4c(c3)c3ccccc3n4-c3cccc(-c4cccc5ccccc45)c3)c3cccc4c3oc3ccccc34)cc2-c2ccccc2)cc1 ; c1ccc(-c2cc(-c3ccccc3)cc(N(c3ccc4c(c3)c3ccccc3n4-c3cccc(-c4cccc5ccccc45)c3)c3cccc4c3oc3ccccc34)c2)cc1 ; c1ccc(-c2ccc(-c3ccccc3)c(N(c3ccc4c(c3)c3ccccc3n4-c3cccc(-c4cccc5ccccc45)c3)c3cccc4c3oc3ccccc34)c2)cc1 ; c1ccc(-c2ccc(N(c3ccc4c(c3)c3ccccc3n4-c3cccc(-c4cccc5ccccc45)c3)c3cccc4c3oc3ccccc34)c(-c3ccccc3)c2)cc1]

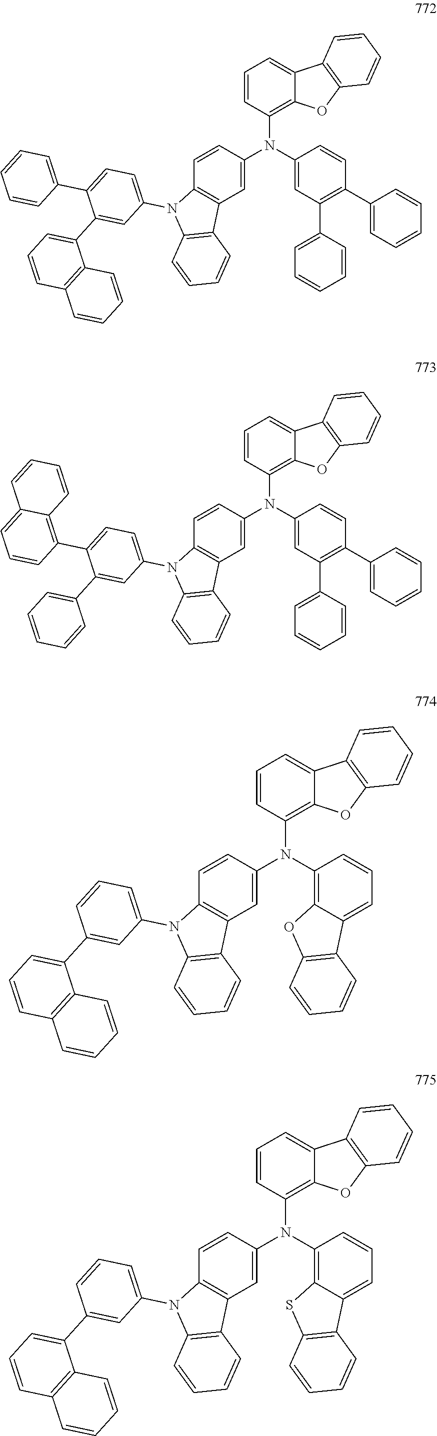 [SMILES: c1ccc(-c2ccc(N(c3ccc4c(c3)c3ccccc3n4-c3ccc(-c4ccccc4)c(-c4cccc5ccccc45)c3)c3cccc4c3oc3ccccc34)cc2-c2ccccc2)cc1 ; c1ccc(-c2ccc(N(c3ccc4c(c3)c3ccccc3n4-c3ccc(-c4cccc5ccccc45)c(-c4ccccc4)c3)c3cccc4c3oc3ccccc34)cc2-c2ccccc2)cc1 ; c1cc(-c2cccc3ccccc23)cc(-n2c3ccccc3c3cc(N(c4cccc5c4oc4ccccc45)c4cccc5c4oc4ccccc45)ccc32)c1 ; c1cc(-c2cccc3ccccc23)cc(-n2c3ccccc3c3cc(N(c4cccc5c4oc4ccccc45)c4cccc5c4sc4ccccc45)ccc32)c1]

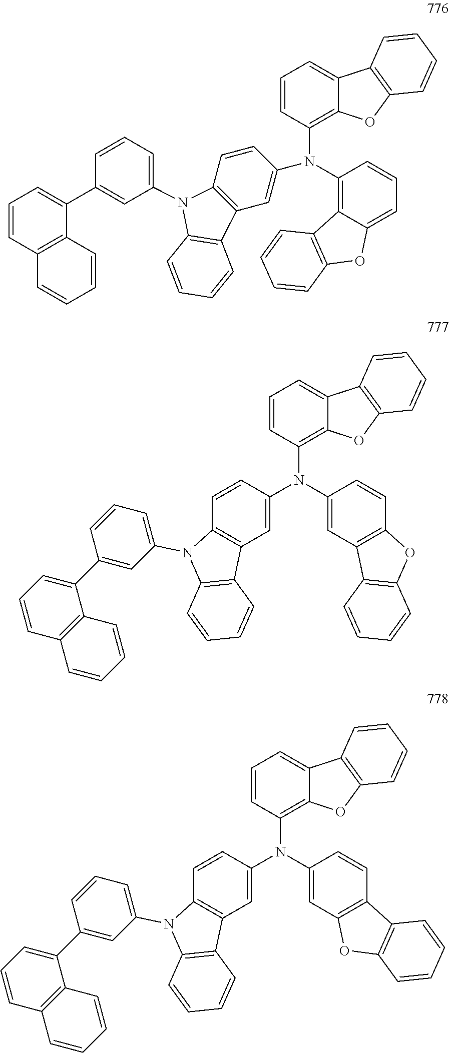 [SMILES: c1cc(-c2cccc3ccccc23)cc(-n2c3ccccc3c3cc(N(c4cccc5c4oc4ccccc45)c4cccc5oc6ccccc6c45)ccc32)c1 ; c1cc(-c2cccc3ccccc23)cc(-n2c3ccccc3c3cc(N(c4ccc5oc6ccccc6c5c4)c4cccc5c4oc4ccccc45)ccc32)c1 ; c1cc(-c2cccc3ccccc23)cc(-n2c3ccccc3c3cc(N(c4ccc5c(c4)oc4ccccc45)c4cccc5c4oc4ccccc45)ccc32)c1]

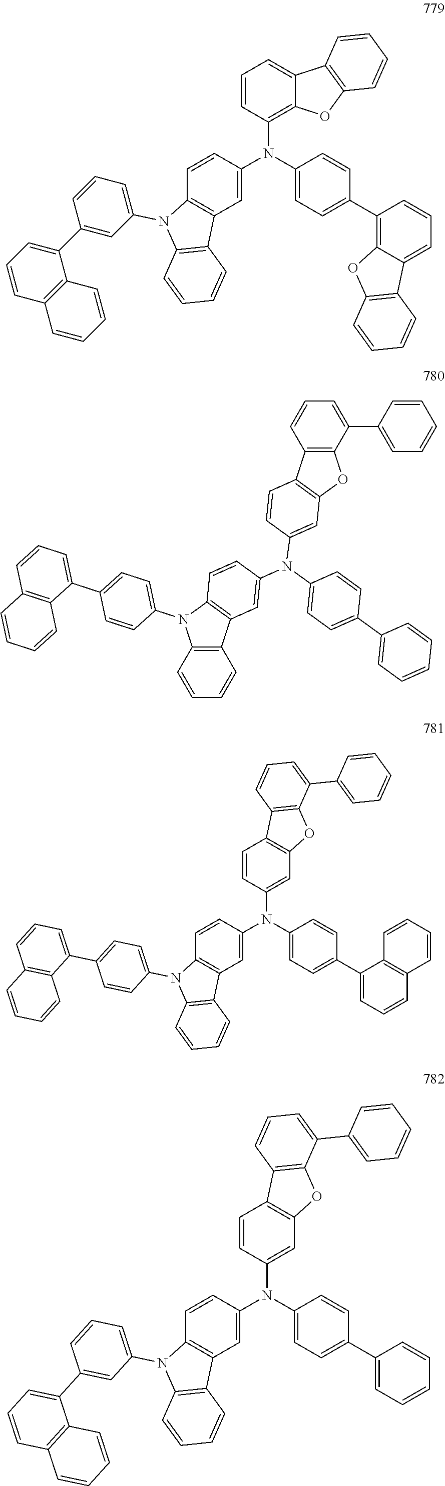 [SMILES: c1cc(-c2cccc3ccccc23)cc(-n2c3ccccc3c3cc(N(c4ccc(-c5cccc6c5oc5ccccc56)cc4)c4cccc5c4oc4ccccc45)ccc32)c1 ; c1ccc(-c2ccc(N(c3ccc4c(c3)oc3c(-c5ccccc5)cccc34)c3ccc4c(c3)c3ccccc3n4-c3ccc(-c4cccc5ccccc45)cc3)cc2)cc1 ; c1ccc(-c2cccc3c2oc2cc(N(c4ccc(-c5cccc6ccccc56)cc4)c4ccc5c(c4)c4ccccc4n5-c4ccc(-c5cccc6ccccc56)cc4)ccc23)cc1 ; c1ccc(-c2ccc(N(c3ccc4c(c3)oc3c(-c5ccccc5)cccc34)c3ccc4c(c3)c3ccccc3n4-c3cccc(-c4cccc5ccccc45)c3)cc2)cc1]

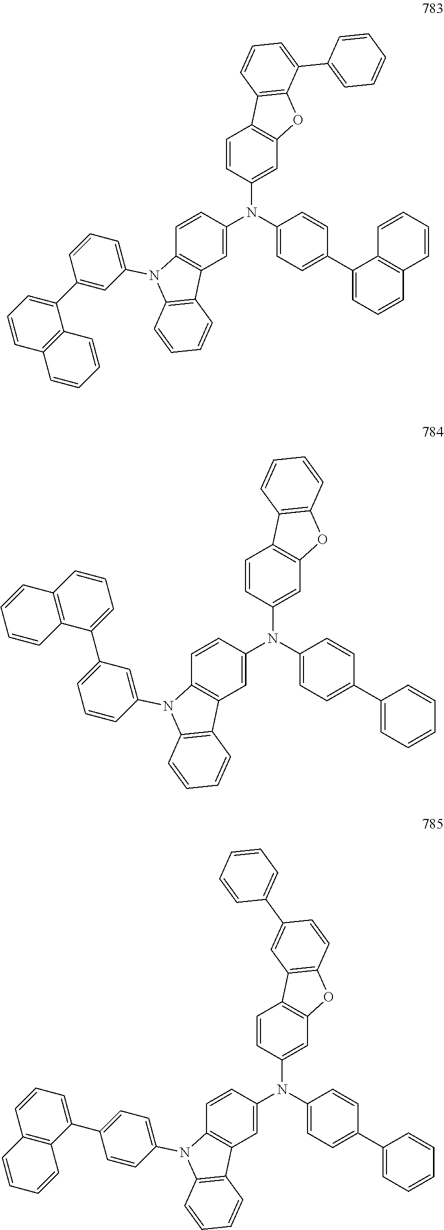 [SMILES: c1ccc(-c2cccc3c2oc2cc(N(c4ccc(-c5cccc6ccccc56)cc4)c4ccc5c(c4)c4ccccc4n5-c4cccc(-c5cccc6ccccc56)c4)ccc23)cc1 ; c1ccc(-c2ccc(N(c3ccc4c(c3)oc3ccccc34)c3ccc4c(c3)c3ccccc3n4-c3cccc(-c4cccc5ccccc45)c3)cc2)cc1 ; c1ccc(-c2ccc(N(c3ccc4c(c3)oc3ccc(-c5ccccc5)cc34)c3ccc4c(c3)c3ccccc3n4-c3ccc(-c4cccc5ccccc45)cc3)cc2)cc1]

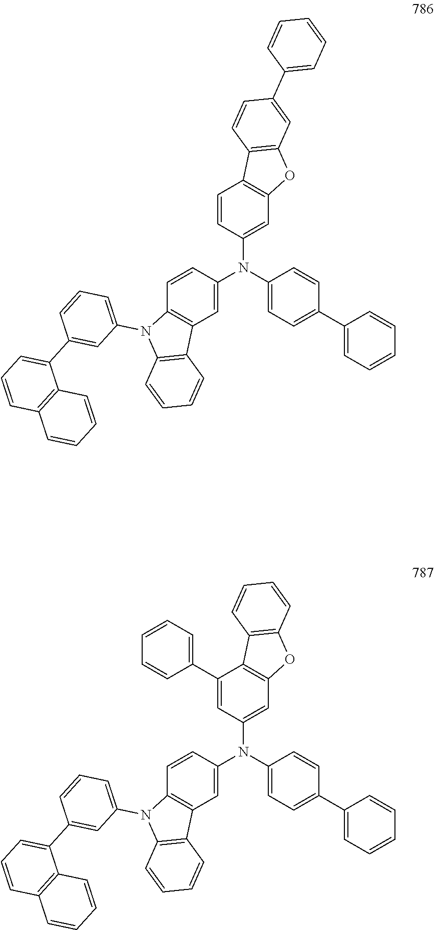 [SMILES: c1ccc(-c2ccc(N(c3ccc4c(c3)oc3cc(-c5ccccc5)ccc34)c3ccc4c(c3)c3ccccc3n4-c3cccc(-c4cccc5ccccc45)c3)cc2)cc1 ; c1ccc(-c2ccc(N(c3cc(-c4ccccc4)c4c(c3)oc3ccccc34)c3ccc4c(c3)c3ccccc3n4-c3cccc(-c4cccc5ccccc45)c3)cc2)cc1]

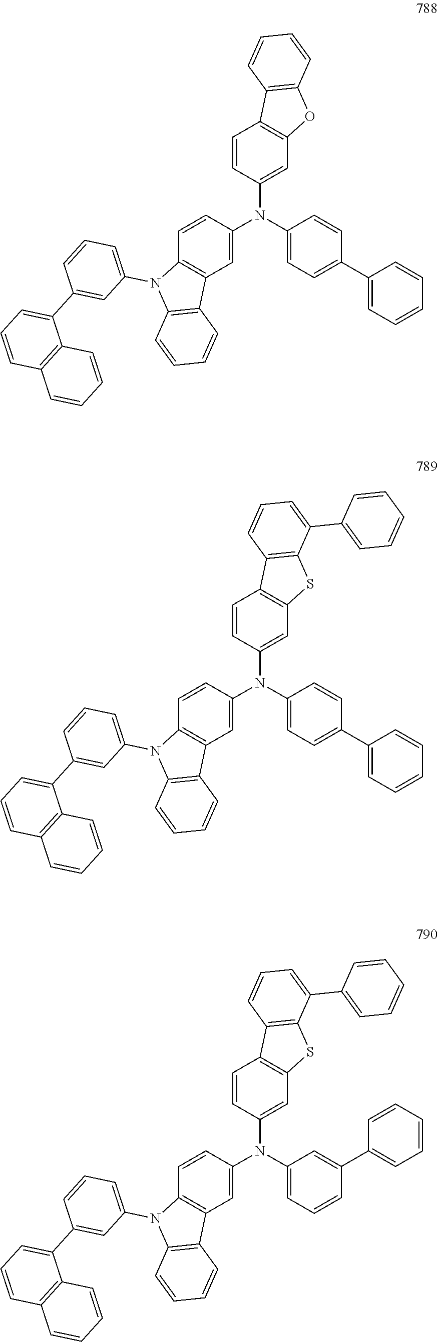 [SMILES: c1ccc(-c2ccc(N(c3ccc4c(c3)oc3ccccc34)c3ccc4c(c3)c3ccccc3n4-c3cccc(-c4cccc5ccccc45)c3)cc2)cc1 ; c1ccc(-c2ccc(N(c3ccc4c(c3)sc3c(-c5ccccc5)cccc34)c3ccc4c(c3)c3ccccc3n4-c3cccc(-c4cccc5ccccc45)c3)cc2)cc1 ; c1ccc(-c2cccc(N(c3ccc4c(c3)sc3c(-c5ccccc5)cccc34)c3ccc4c(c3)c3ccccc3n4-c3cccc(-c4cccc5ccccc45)c3)c2)cc1]

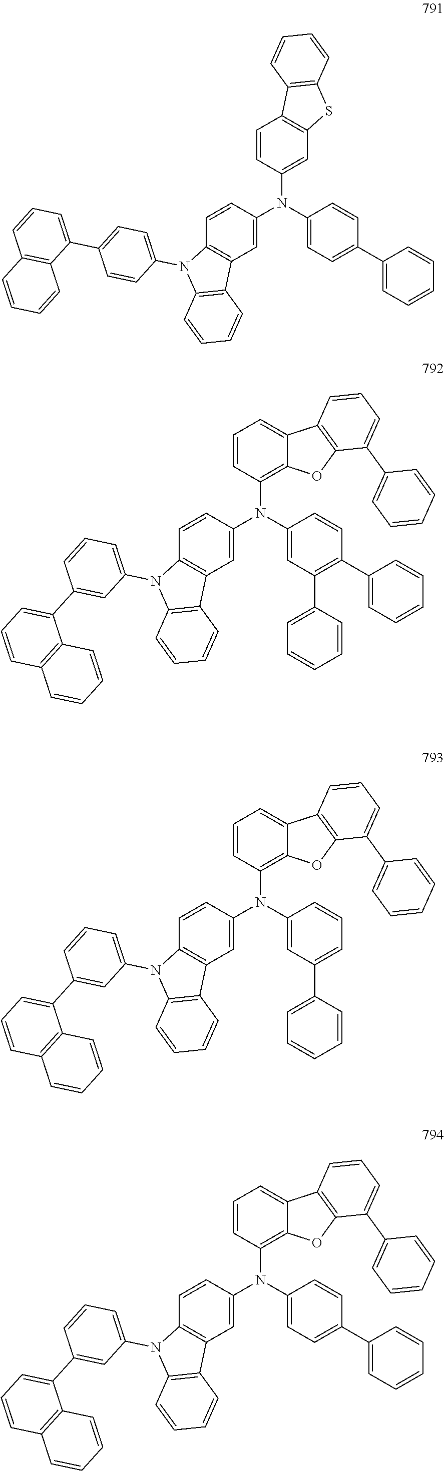 [SMILES: c1ccc(-c2ccc(N(c3ccc4c(c3)sc3ccccc34)c3ccc4c(c3)c3ccccc3n4-c3ccc(-c4cccc5ccccc45)cc3)cc2)cc1 ; c1ccc(-c2ccc(N(c3ccc4c(c3)c3ccccc3n4-c3cccc(-c4cccc5ccccc45)c3)c3cccc4c3oc3c(-c5ccccc5)cccc34)cc2-c2ccccc2)cc1 ; c1ccc(-c2cccc(N(c3ccc4c(c3)c3ccccc3n4-c3cccc(-c4cccc5ccccc45)c3)c3cccc4c3oc3c(-c5ccccc5)cccc34)c2)cc1 ; c1ccc(-c2ccc(N(c3ccc4c(c3)c3ccccc3n4-c3cccc(-c4cccc5ccccc45)c3)c3cccc4c3oc3c(-c5ccccc5)cccc34)cc2)cc1]

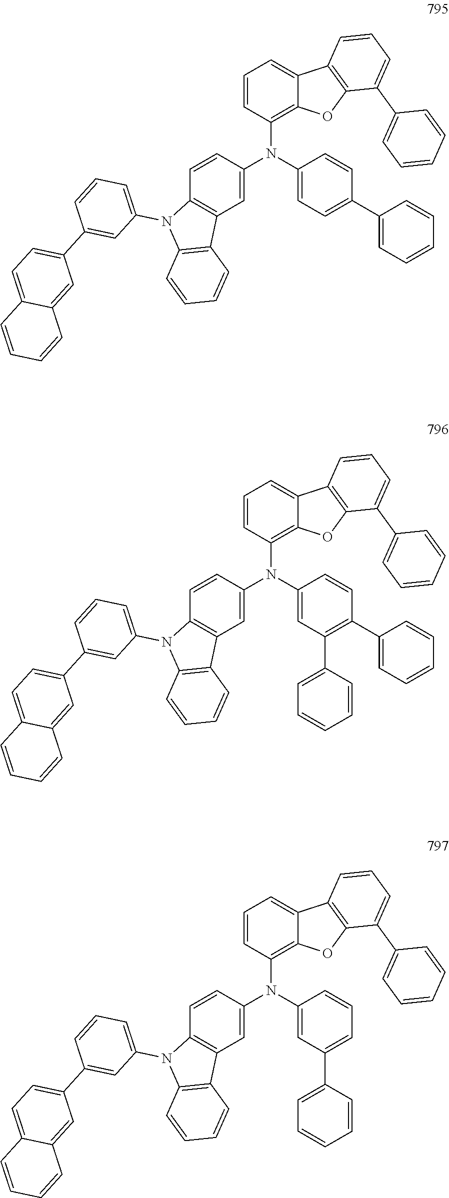 [SMILES: c1ccc(-c2ccc(N(c3ccc4c(c3)c3ccccc3n4-c3cccc(-c4ccc5ccccc5c4)c3)c3cccc4c3oc3c(-c5ccccc5)cccc34)cc2)cc1 ; c1ccc(-c2ccc(N(c3ccc4c(c3)c3ccccc3n4-c3cccc(-c4ccc5ccccc5c4)c3)c3cccc4c3oc3c(-c5ccccc5)cccc34)cc2-c2ccccc2)cc1 ; c1ccc(-c2cccc(N(c3ccc4c(c3)c3ccccc3n4-c3cccc(-c4ccc5ccccc5c4)c3)c3cccc4c3oc3c(-c5ccccc5)cccc34)c2)cc1]

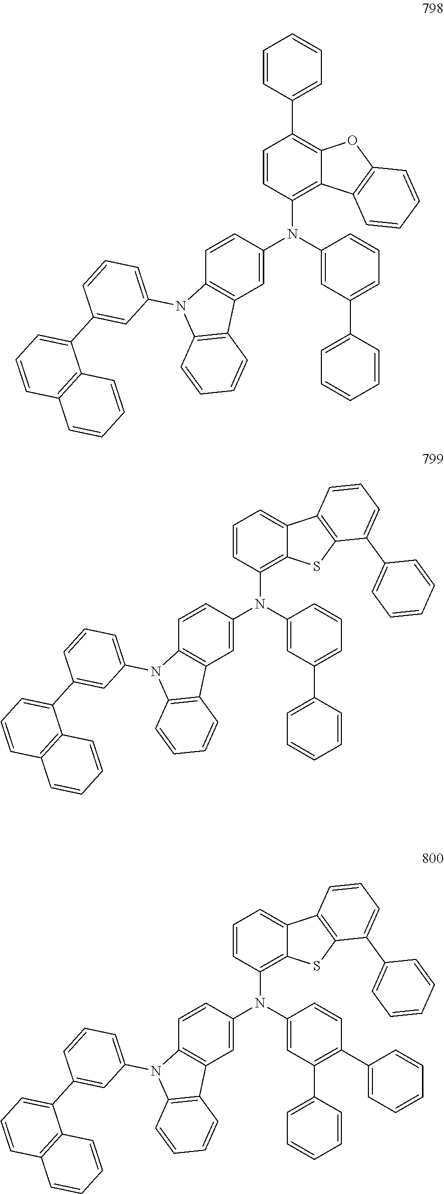 [SMILES: c1ccc(-c2cccc(N(c3ccc4c(c3)c3ccccc3n4-c3cccc(-c4cccc5ccccc45)c3)c3ccc(-c4ccccc4)c4oc5ccccc5c34)c2)cc1 ; c1ccc(-c2cccc(N(c3ccc4c(c3)c3ccccc3n4-c3cccc(-c4cccc5ccccc45)c3)c3cccc4c3sc3c(-c5ccccc5)cccc34)c2)cc1 ; c1ccc(-c2ccc(N(c3ccc4c(c3)c3ccccc3n4-c3cccc(-c4cccc5ccccc45)c3)c3cccc4c3sc3c(-c5ccccc5)cccc34)cc2-c2ccccc2)cc1]

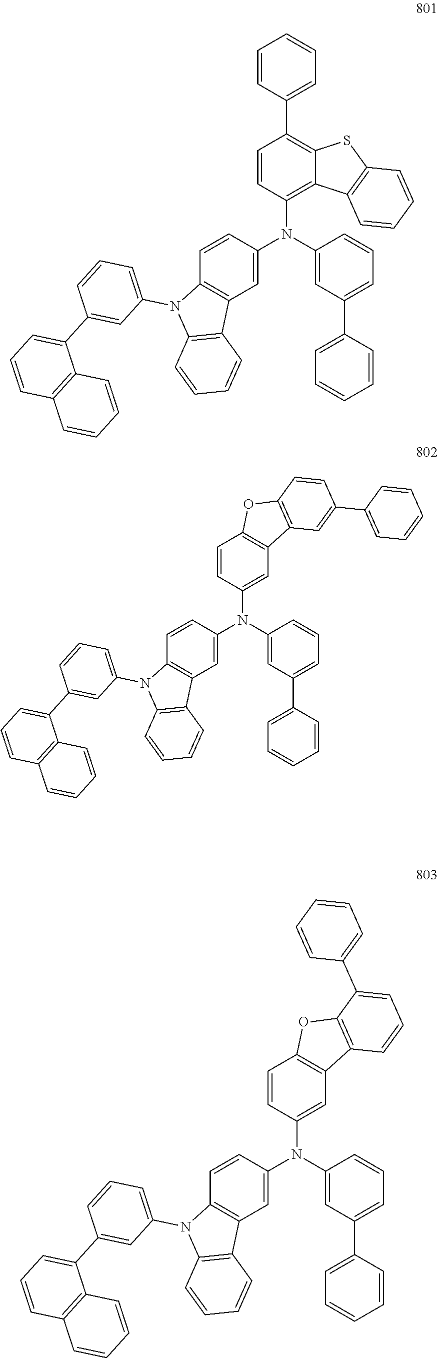 [SMILES: c1ccc(-c2cccc(N(c3ccc4c(c3)c3ccccc3n4-c3cccc(-c4cccc5ccccc45)c3)c3ccc(-c4ccccc4)c4sc5ccccc5c34)c2)cc1 ; c1ccc(-c2cccc(N(c3ccc4oc5ccc(-c6ccccc6)cc5c4c3)c3ccc4c(c3)c3ccccc3n4-c3cccc(-c4cccc5ccccc45)c3)c2)cc1 ; c1ccc(-c2cccc(N(c3ccc4oc5c(-c6ccccc6)cccc5c4c3)c3ccc4c(c3)c3ccccc3n4-c3cccc(-c4cccc5ccccc45)c3)c2)cc1]

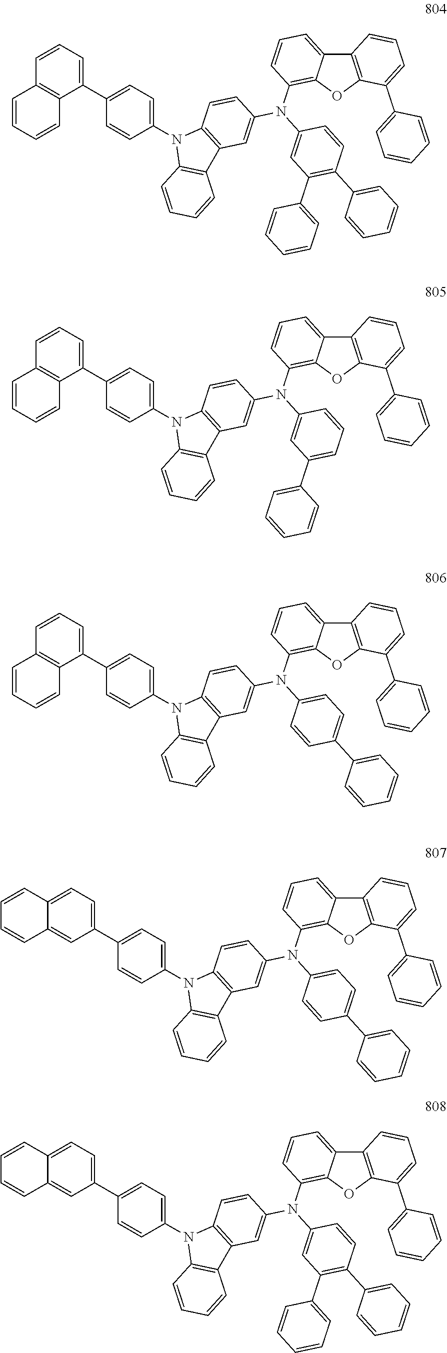

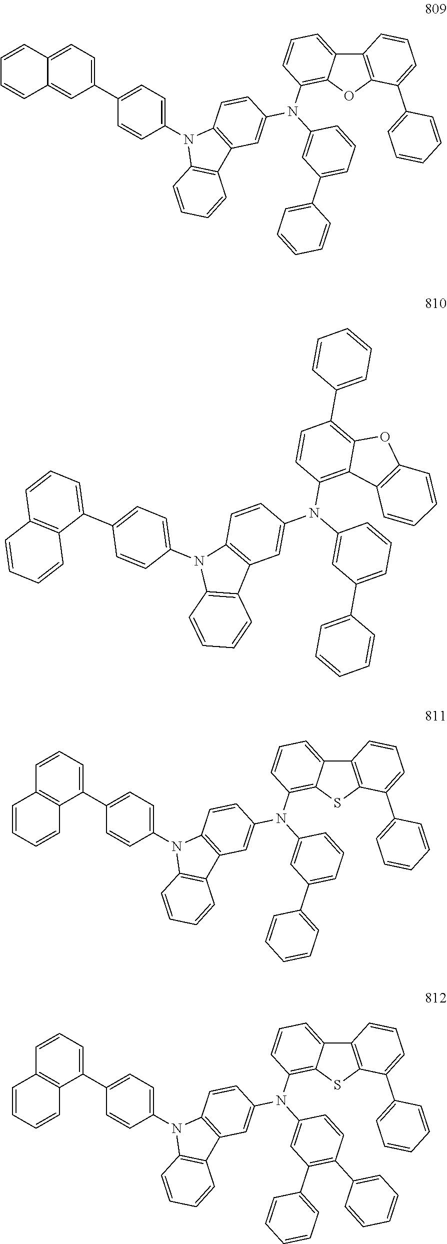 [SMILES: c1ccc(-c2cccc(N(c3ccc4c(c3)c3ccccc3n4-c3ccc(-c4ccc5ccccc5c4)cc3)c3cccc4c3oc3c(-c5ccccc5)cccc34)c2)cc1 ; c1ccc(-c2cccc(N(c3ccc4c(c3)c3ccccc3n4-c3ccc(-c4cccc5ccccc45)cc3)c3ccc(-c4ccccc4)c4oc5ccccc5c34)c2)cc1 ; c1ccc(-c2cccc(N(c3ccc4c(c3)c3ccccc3n4-c3ccc(-c4cccc5ccccc45)cc3)c3cccc4c3sc3c(-c5ccccc5)cccc34)c2)cc1 ; c1ccc(-c2ccc(N(c3ccc4c(c3)c3ccccc3n4-c3ccc(-c4cccc5ccccc45)cc3)c3cccc4c3sc3c(-c5ccccc5)cccc34)cc2-c2ccccc2)cc1]

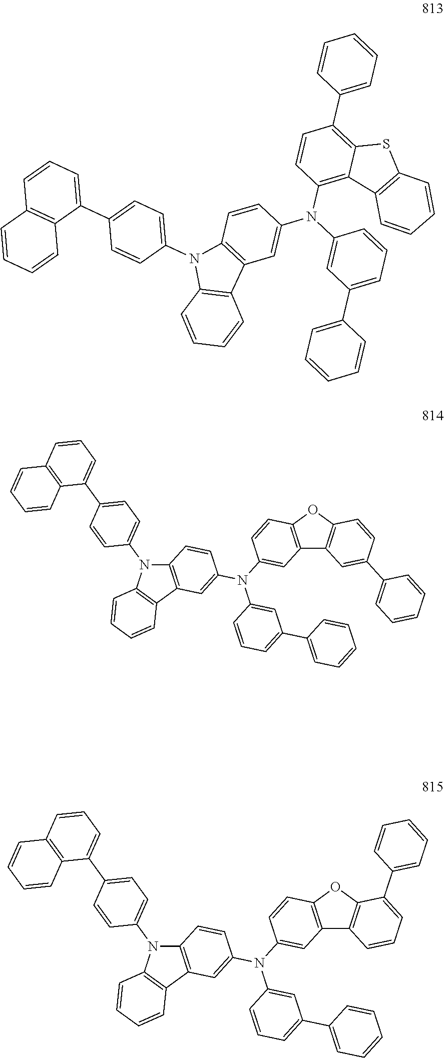 [SMILES: c1ccc(-c2cccc(N(c3ccc4c(c3)c3ccccc3n4-c3ccc(-c4cccc5ccccc45)cc3)c3ccc(-c4ccccc4)c4sc5ccccc5c34)c2)cc1 ; c1ccc(-c2cccc(N(c3ccc4oc5ccc(-c6ccccc6)cc5c4c3)c3ccc4c(c3)c3ccccc3n4-c3ccc(-c4cccc5ccccc45)cc3)c2)cc1 ; c1ccc(-c2cccc(N(c3ccc4oc5c(-c6ccccc6)cccc5c4c3)c3ccc4c(c3)c3ccccc3n4-c3ccc(-c4cccc5ccccc45)cc3)c2)cc1]

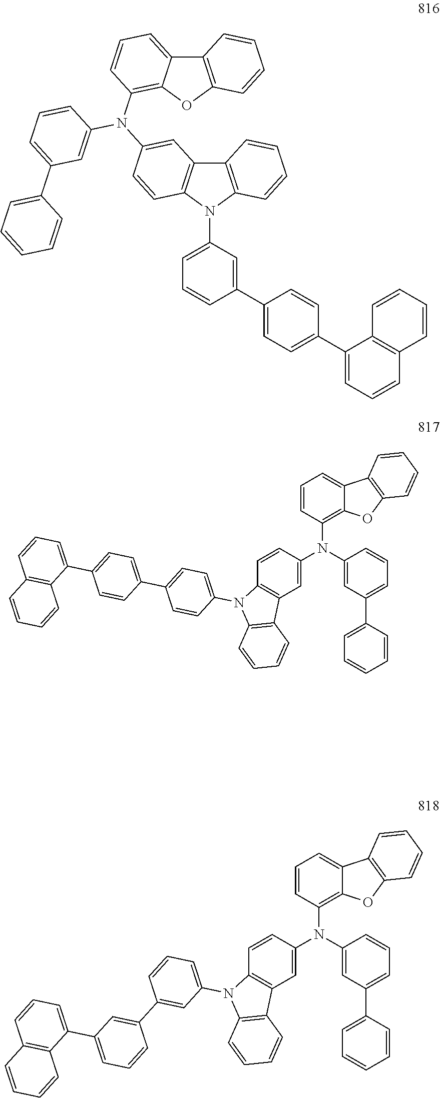 [SMILES: c1ccc(-c2cccc(N(c3ccc4c(c3)c3ccccc3n4-c3cccc(-c4ccc(-c5cccc6ccccc56)cc4)c3)c3cccc4c3oc3ccccc34)c2)cc1 ; c1ccc(-c2cccc(N(c3ccc4c(c3)c3ccccc3n4-c3ccc(-c4ccc(-c5cccc6ccccc56)cc4)cc3)c3cccc4c3oc3ccccc34)c2)cc1 ; c1ccc(-c2cccc(N(c3ccc4c(c3)c3ccccc3n4-c3cccc(-c4cccc(-c5cccc6ccccc56)c4)c3)c3cccc4c3oc3ccccc34)c2)cc1]

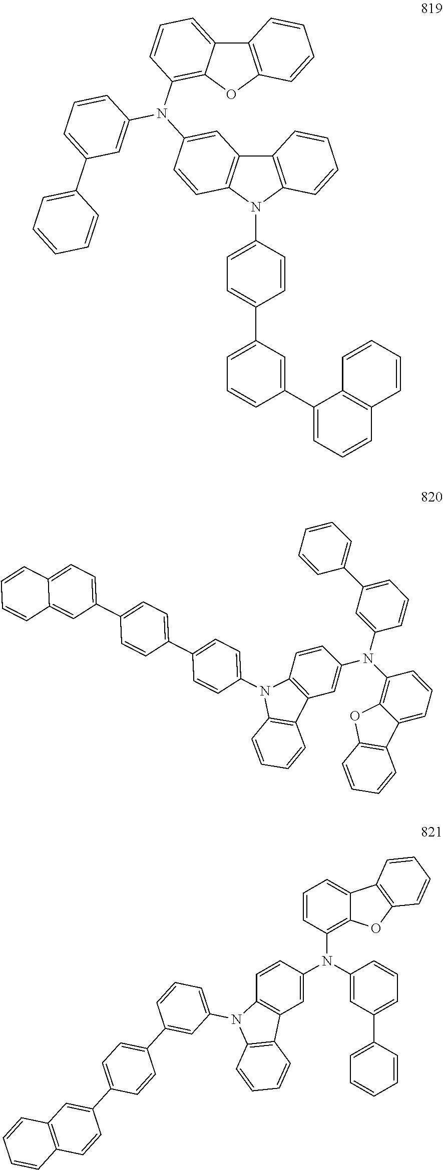 [SMILES: c1ccc(-c2cccc(N(c3ccc4c(c3)c3ccccc3n4-c3ccc(-c4cccc(-c5cccc6ccccc56)c4)cc3)c3cccc4c3oc3ccccc34)c2)cc1 ; c1ccc(-c2cccc(N(c3ccc4c(c3)c3ccccc3n4-c3ccc(-c4ccc(-c5ccc6ccccc6c5)cc4)cc3)c3cccc4c3oc3ccccc34)c2)cc1 ; c1ccc(-c2cccc(N(c3ccc4c(c3)c3ccccc3n4-c3cccc(-c4ccc(-c5ccc6ccccc6c5)cc4)c3)c3cccc4c3oc3ccccc34)c2)cc1]

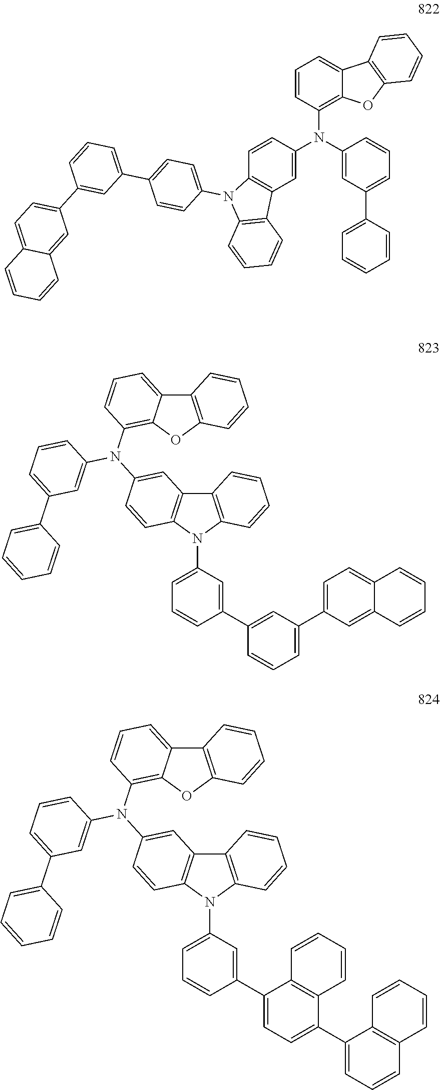 [SMILES: c1ccc(-c2cccc(N(c3ccc4c(c3)c3ccccc3n4-c3ccc(-c4cccc(-c5ccc6ccccc6c5)c4)cc3)c3cccc4c3oc3ccccc34)c2)cc1 ; c1ccc(-c2cccc(N(c3ccc4c(c3)c3ccccc3n4-c3cccc(-c4cccc(-c5ccc6ccccc6c5)c4)c3)c3cccc4c3oc3ccccc34)c2)cc1 ; c1ccc(-c2cccc(N(c3ccc4c(c3)c3ccccc3n4-c3cccc(-c4ccc(-c5cccc6ccccc56)c5ccccc45)c3)c3cccc4c3oc3ccccc34)c2)cc1]

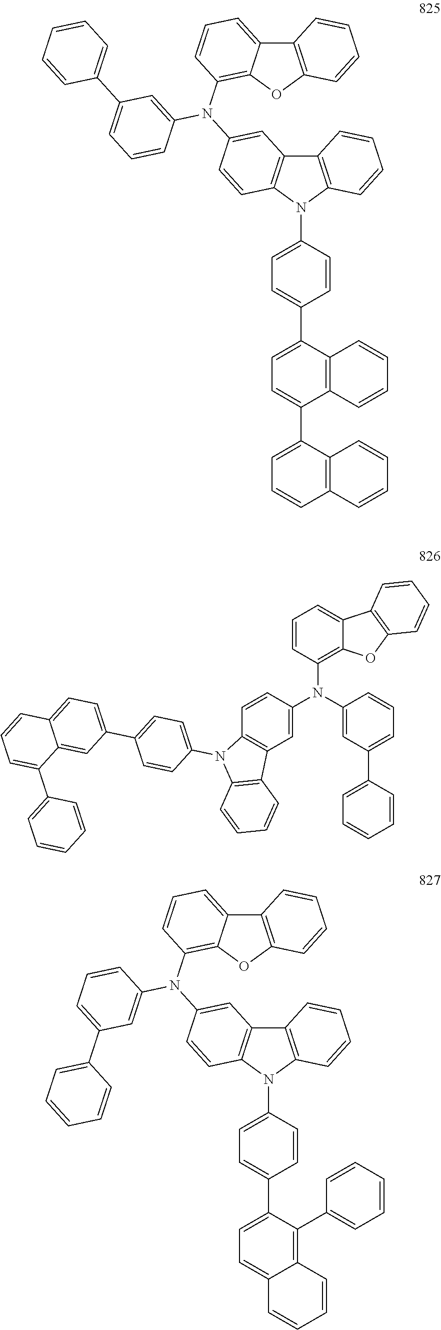 [SMILES: c1ccc(-c2cccc(N(c3ccc4c(c3)c3ccccc3n4-c3ccc(-c4ccc(-c5cccc6ccccc56)c5ccccc45)cc3)c3cccc4c3oc3ccccc34)c2)cc1 ; c1ccc(-c2cccc(N(c3ccc4c(c3)c3ccccc3n4-c3ccc(-c4ccc5cccc(-c6ccccc6)c5c4)cc3)c3cccc4c3oc3ccccc34)c2)cc1 ; c1ccc(-c2cccc(N(c3ccc4c(c3)c3ccccc3n4-c3ccc(-c4ccc5ccccc5c4-c4ccccc4)cc3)c3cccc4c3oc3ccccc34)c2)cc1]

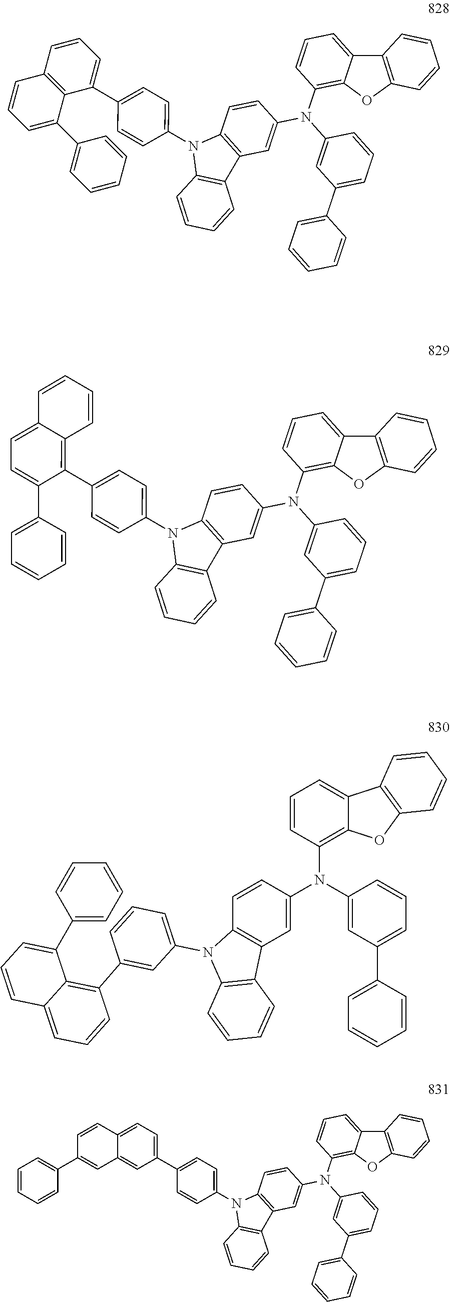 [SMILES: c1ccc(-c2cccc(N(c3ccc4c(c3)c3ccccc3n4-c3ccc(-c4cccc5cccc(-c6ccccc6)c45)cc3)c3cccc4c3oc3ccccc34)c2)cc1 ; c1ccc(-c2cccc(N(c3ccc4c(c3)c3ccccc3n4-c3ccc(-c4c(-c5ccccc5)ccc5ccccc45)cc3)c3cccc4c3oc3ccccc34)c2)cc1 ; c1ccc(-c2cccc(N(c3ccc4c(c3)c3ccccc3n4-c3cccc(-c4cccc5cccc(-c6ccccc6)c45)c3)c3cccc4c3oc3ccccc34)c2)cc1 ; c1ccc(-c2cccc(N(c3ccc4c(c3)c3ccccc3n4-c3ccc(-c4ccc5ccc(-c6ccccc6)cc5c4)cc3)c3cccc4c3oc3ccccc34)c2)cc1]

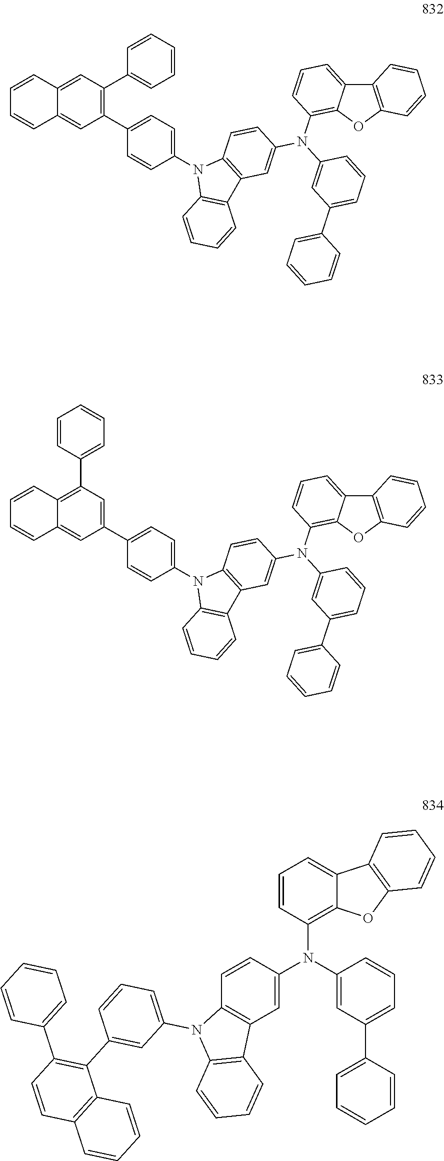 [SMILES: c1ccc(-c2cccc(N(c3ccc4c(c3)c3ccccc3n4-c3ccc(-c4cc5ccccc5cc4-c4ccccc4)cc3)c3cccc4c3oc3ccccc34)c2)cc1 ; c1ccc(-c2cccc(N(c3ccc4c(c3)c3ccccc3n4-c3ccc(-c4cc(-c5ccccc5)c5ccccc5c4)cc3)c3cccc4c3oc3ccccc34)c2)cc1 ; c1ccc(-c2cccc(N(c3ccc4c(c3)c3ccccc3n4-c3cccc(-c4c(-c5ccccc5)ccc5ccccc45)c3)c3cccc4c3oc3ccccc34)c2)cc1]

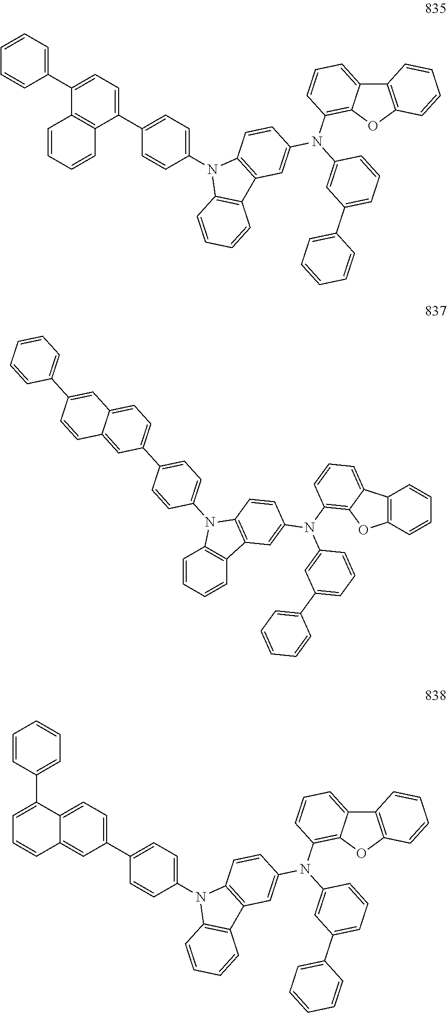 [SMILES: c1ccc(-c2cccc(N(c3ccc4c(c3)c3ccccc3n4-c3ccc(-c4ccc(-c5ccccc5)c5ccccc45)cc3)c3cccc4c3oc3ccccc34)c2)cc1 ; c1ccc(-c2cccc(N(c3ccc4c(c3)c3ccccc3n4-c3ccc(-c4ccc5cc(-c6ccccc6)ccc5c4)cc3)c3cccc4c3oc3ccccc34)c2)cc1 ; c1ccc(-c2cccc(N(c3ccc4c(c3)c3ccccc3n4-c3ccc(-c4ccc5c(-c6ccccc6)cccc5c4)cc3)c3cccc4c3oc3ccccc34)c2)cc1]

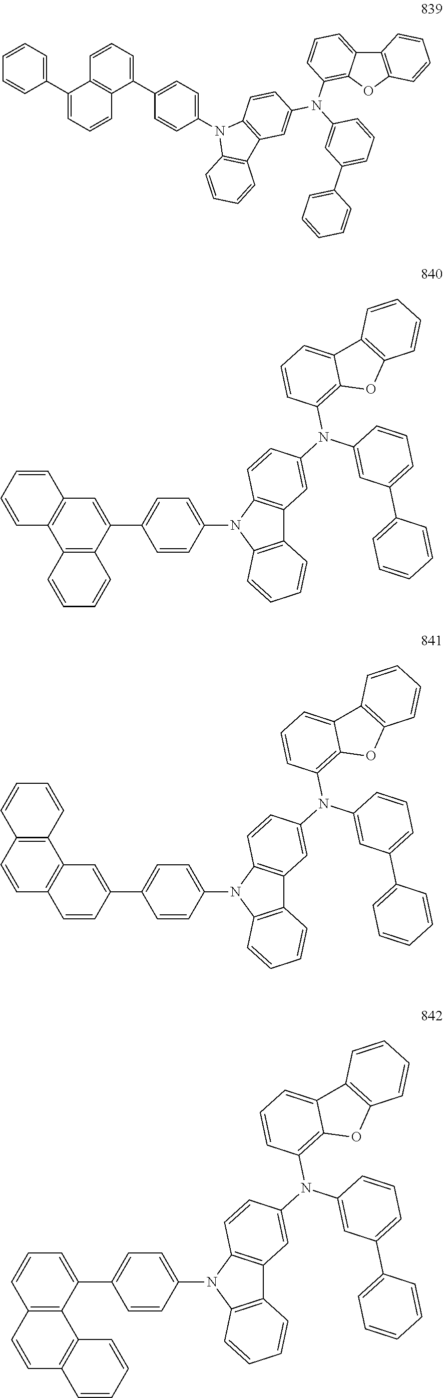 [SMILES: c1ccc(-c2cccc(N(c3ccc4c(c3)c3ccccc3n4-c3ccc(-c4cccc5c(-c6ccccc6)cccc45)cc3)c3cccc4c3oc3ccccc34)c2)cc1 ; c1ccc(-c2cccc(N(c3ccc4c(c3)c3ccccc3n4-c3ccc(-c4cc5ccccc5c5ccccc45)cc3)c3cccc4c3oc3ccccc34)c2)cc1 ; c1ccc(-c2cccc(N(c3ccc4c(c3)c3ccccc3n4-c3ccc(-c4ccc5ccc6ccccc6c5c4)cc3)c3cccc4c3oc3ccccc34)c2)cc1 ; c1ccc(-c2cccc(N(c3ccc4c(c3)c3ccccc3n4-c3ccc(-c4cccc5ccc6ccccc6c45)cc3)c3cccc4c3oc3ccccc34)c2)cc1]

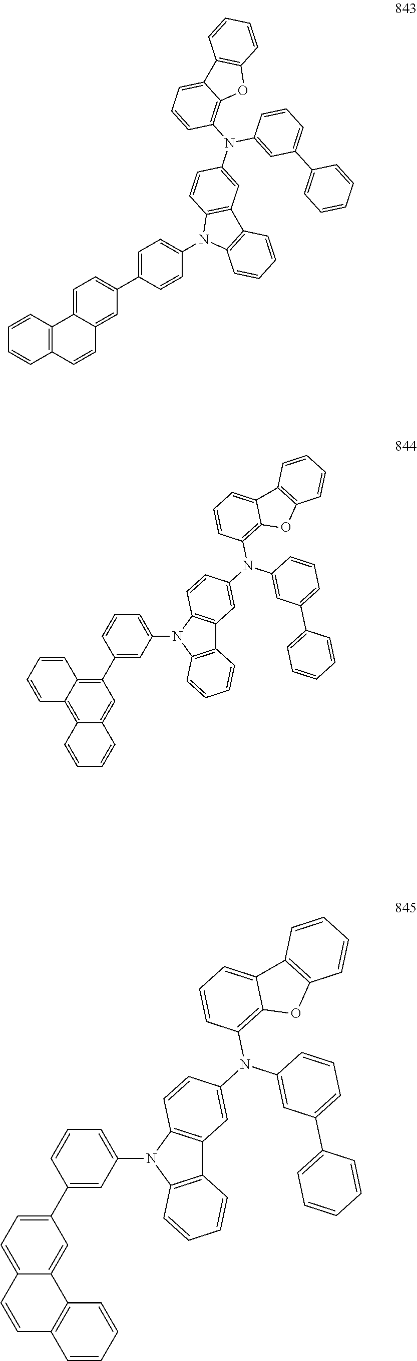 [SMILES: c1ccc(-c2cccc(N(c3ccc4c(c3)c3ccccc3n4-c3ccc(-c4ccc5c(ccc6ccccc65)c4)cc3)c3cccc4c3oc3ccccc34)c2)cc1 ; c1ccc(-c2cccc(N(c3ccc4c(c3)c3ccccc3n4-c3cccc(-c4cc5ccccc5c5ccccc45)c3)c3cccc4c3oc3ccccc34)c2)cc1 ; c1ccc(-c2cccc(N(c3ccc4c(c3)c3ccccc3n4-c3cccc(-c4ccc5ccc6ccccc6c5c4)c3)c3cccc4c3oc3ccccc34)c2)cc1]

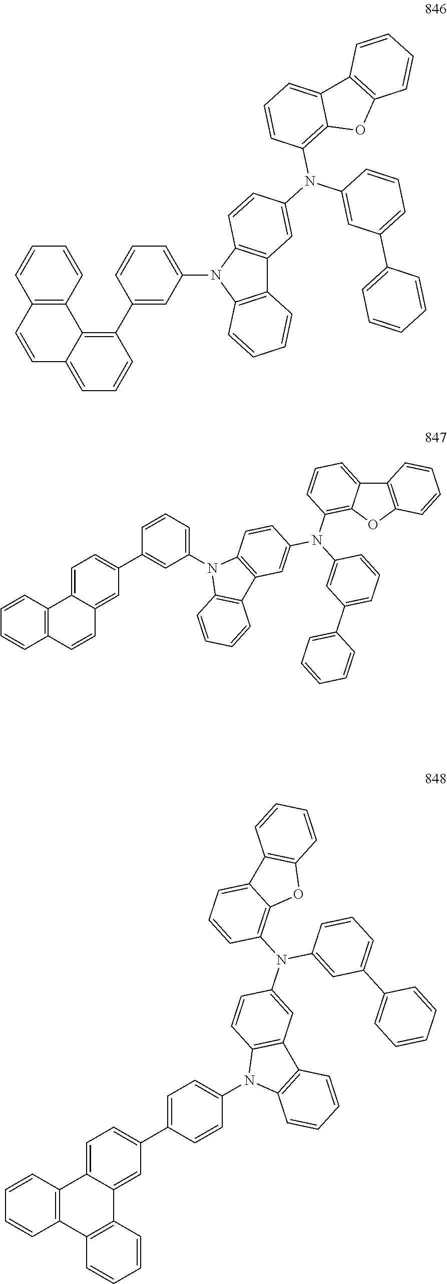 [SMILES: c1ccc(-c2cccc(N(c3ccc4c(c3)c3ccccc3n4-c3cccc(-c4cccc5ccc6ccccc6c45)c3)c3cccc4c3oc3ccccc34)c2)cc1 ; c1ccc(-c2cccc(N(c3ccc4c(c3)c3ccccc3n4-c3cccc(-c4ccc5c(ccc6ccccc65)c4)c3)c3cccc4c3oc3ccccc34)c2)cc1 ; c1ccc(-c2cccc(N(c3ccc4c(c3)c3ccccc3n4-c3ccc(-c4ccc5c6ccccc6c6ccccc6c5c4)cc3)c3cccc4c3oc3ccccc34)c2)cc1]

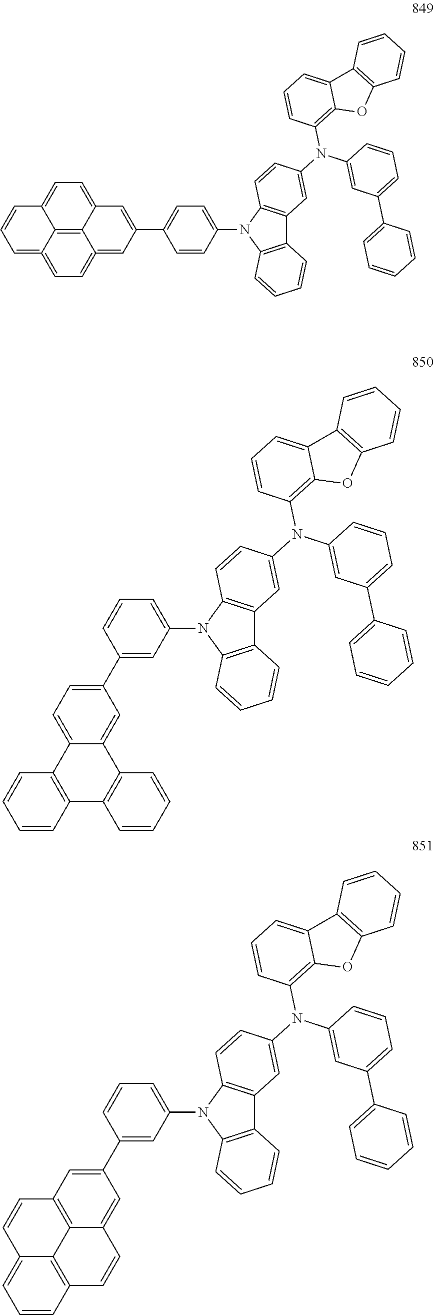

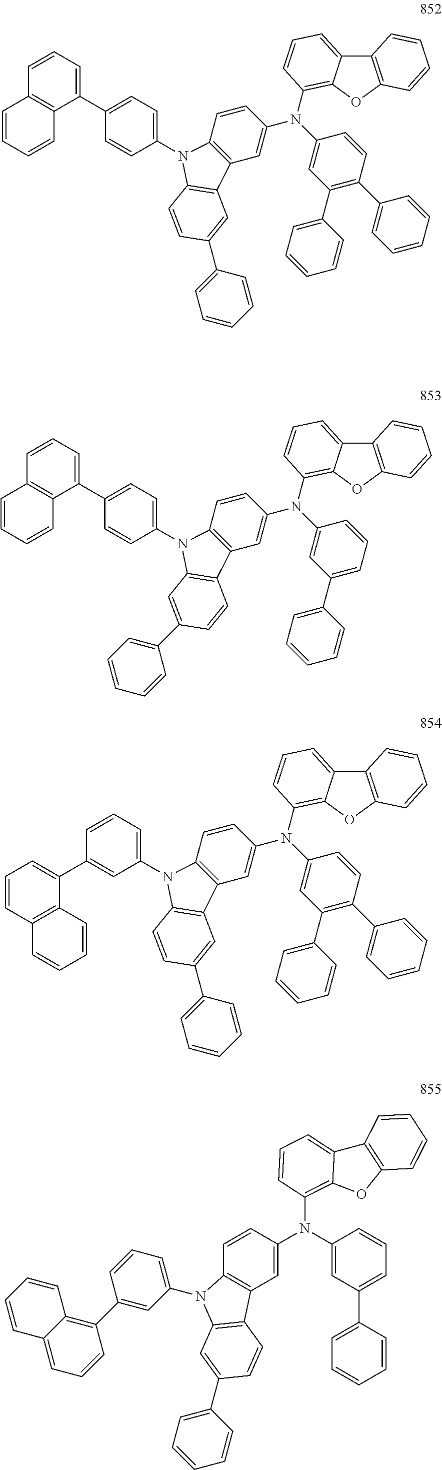 [SMILES: c1ccc(-c2ccc3c(c2)c2cc(N(c4ccc(-c5ccccc5)c(-c5ccccc5)c4)c4cccc5c4oc4ccccc45)ccc2n3-c2ccc(-c3cccc4ccccc34)cc2)cc1 ; c1ccc(-c2cccc(N(c3ccc4c(c3)c3ccc(-c5ccccc5)cc3n4-c3ccc(-c4cccc5ccccc45)cc3)c3cccc4c3oc3ccccc34)c2)cc1 ; c1ccc(-c2ccc3c(c2)c2cc(N(c4ccc(-c5ccccc5)c(-c5ccccc5)c4)c4cccc5c4oc4ccccc45)ccc2n3-c2cccc(-c3cccc4ccccc34)c2)cc1 ; c1ccc(-c2cccc(N(c3ccc4c(c3)c3ccc(-c5ccccc5)cc3n4-c3cccc(-c4cccc5ccccc45)c3)c3cccc4c3oc3ccccc34)c2)cc1]

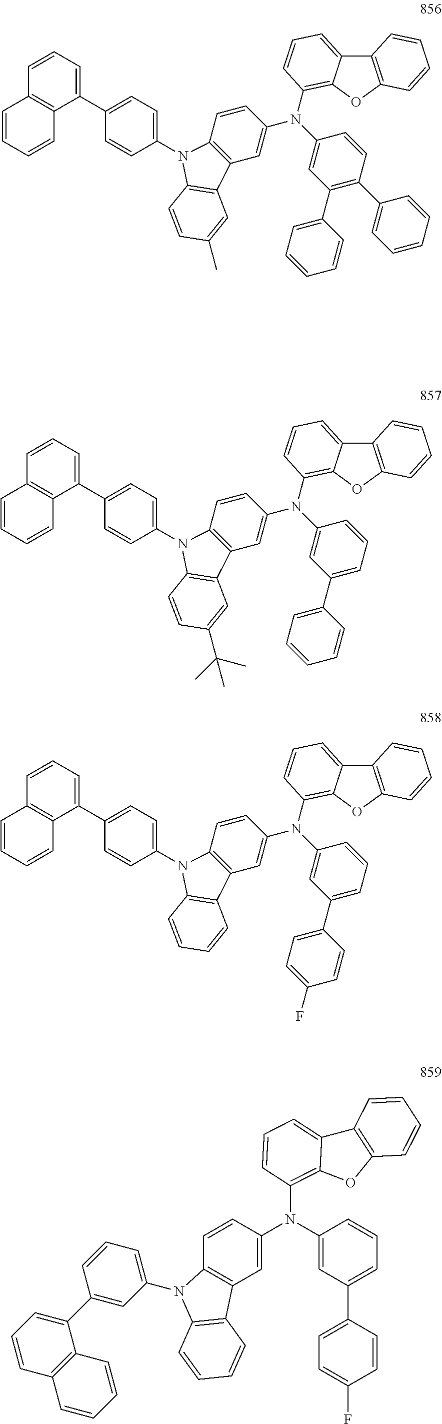 [SMILES: Cc1ccc2c(c1)c1cc(N(c3ccc(-c4ccccc4)c(-c4ccccc4)c3)c3cccc4c3oc3ccccc34)ccc1n2-c1ccc(-c2cccc3ccccc23)cc1 ; CC(C)(C)c1ccc2c(c1)c1cc(N(c3cccc(-c4ccccc4)c3)c3cccc4c3oc3ccccc34)ccc1n2-c1ccc(-c2cccc3ccccc23)cc1 ; Fc1ccc(-c2cccc(N(c3ccc4c(c3)c3ccccc3n4-c3ccc(-c4cccc5ccccc45)cc3)c3cccc4c3oc3ccccc34)c2)cc1 ; Fc1ccc(-c2cccc(N(c3ccc4c(c3)c3ccccc3n4-c3cccc(-c4cccc5ccccc45)c3)c3cccc4c3oc3ccccc34)c2)cc1]

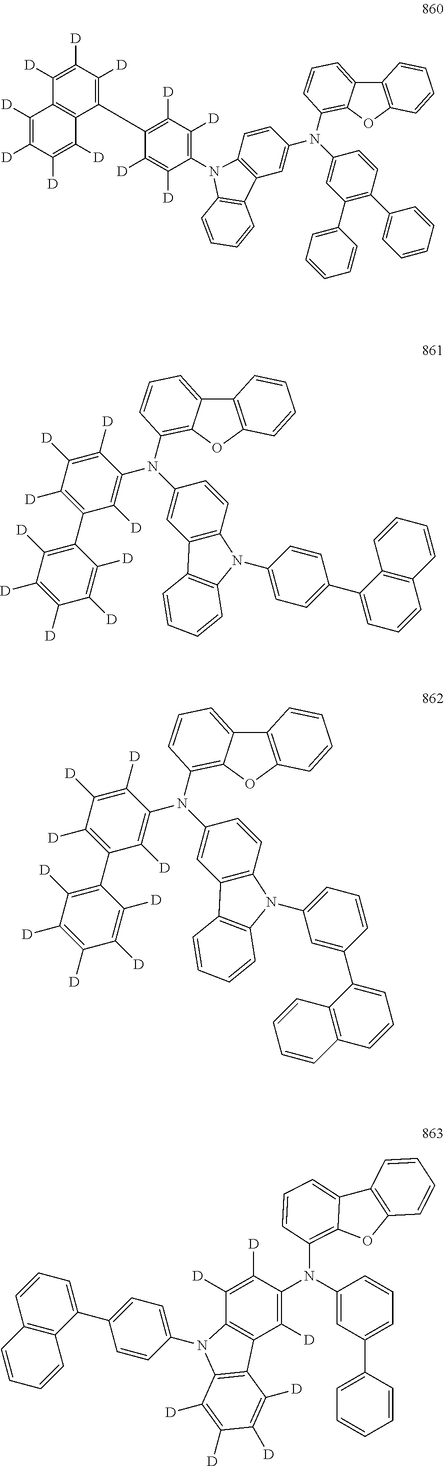

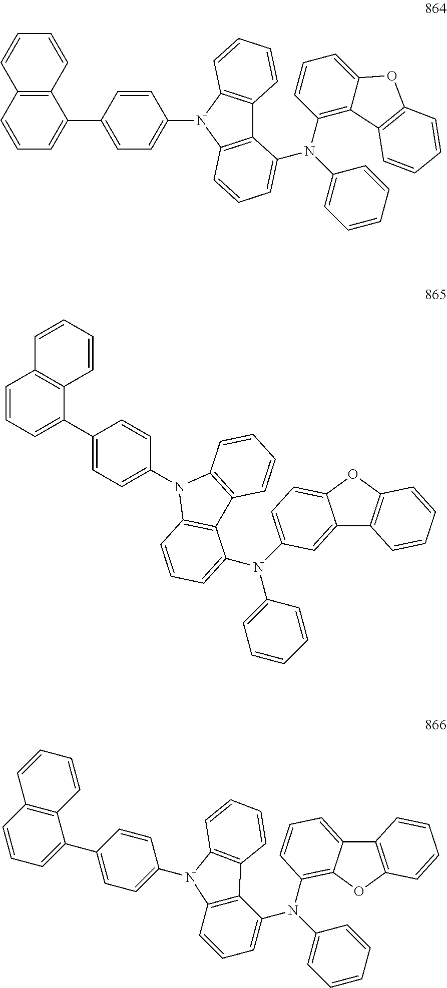 [SMILES: c1ccc(N(c2cccc3oc4ccccc4c23)c2cccc3c2c2ccccc2n3-c2ccc(-c3cccc4ccccc34)cc2)cc1 ; c1ccc(N(c2ccc3oc4ccccc4c3c2)c2cccc3c2c2ccccc2n3-c2ccc(-c3cccc4ccccc34)cc2)cc1 ; c1ccc(N(c2cccc3c2oc2ccccc23)c2cccc3c2c2ccccc2n3-c2ccc(-c3cccc4ccccc34)cc2)cc1]

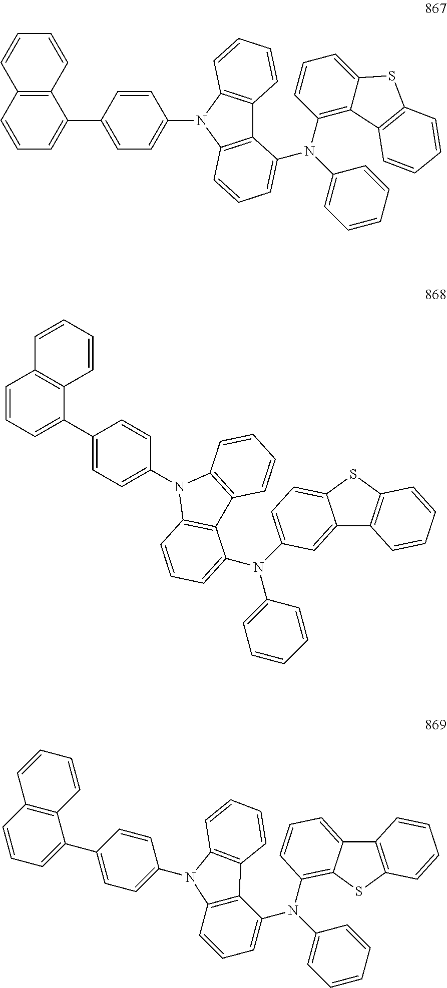 [SMILES: c1ccc(N(c2cccc3sc4ccccc4c23)c2cccc3c2c2ccccc2n3-c2ccc(-c3cccc4ccccc34)cc2)cc1 ; c1ccc(N(c2ccc3sc4ccccc4c3c2)c2cccc3c2c2ccccc2n3-c2ccc(-c3cccc4ccccc34)cc2)cc1 ; c1ccc(N(c2cccc3c2sc2ccccc23)c2cccc3c2c2ccccc2n3-c2ccc(-c3cccc4ccccc34)cc2)cc1]

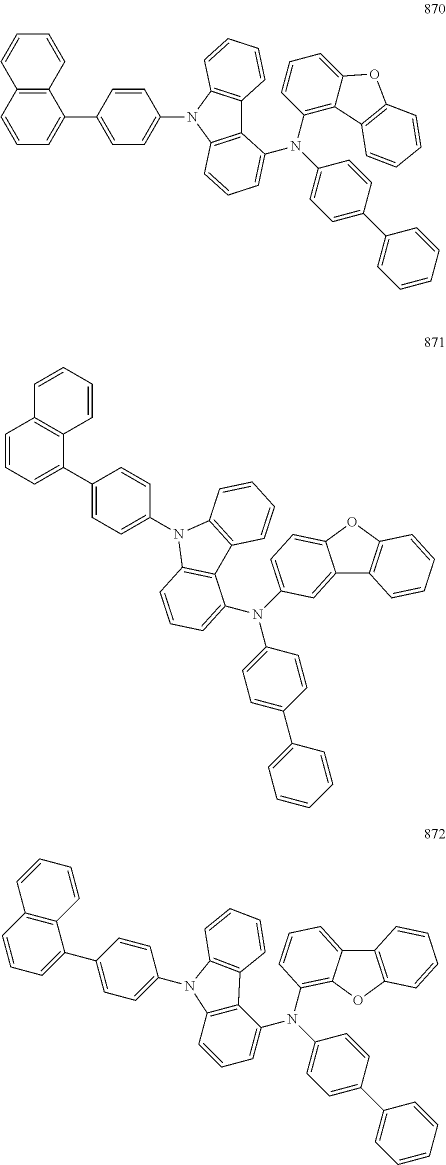 [SMILES: c1ccc(-c2ccc(N(c3cccc4oc5ccccc5c34)c3cccc4c3c3ccccc3n4-c3ccc(-c4cccc5ccccc45)cc3)cc2)cc1 ; c1ccc(-c2ccc(N(c3ccc4oc5ccccc5c4c3)c3cccc4c3c3ccccc3n4-c3ccc(-c4cccc5ccccc45)cc3)cc2)cc1 ; c1ccc(-c2ccc(N(c3cccc4c3oc3ccccc34)c3cccc4c3c3ccccc3n4-c3ccc(-c4cccc5ccccc45)cc3)cc2)cc1]

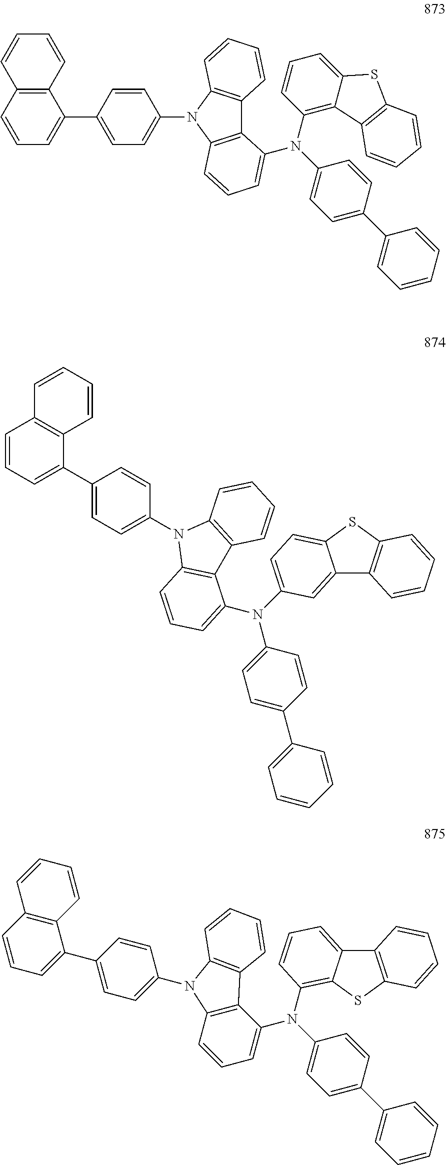 [SMILES: c1ccc(-c2ccc(N(c3cccc4sc5ccccc5c34)c3cccc4c3c3ccccc3n4-c3ccc(-c4cccc5ccccc45)cc3)cc2)cc1 ; c1ccc(-c2ccc(N(c3ccc4sc5ccccc5c4c3)c3cccc4c3c3ccccc3n4-c3ccc(-c4cccc5ccccc45)cc3)cc2)cc1 ; c1ccc(-c2ccc(N(c3cccc4c3sc3ccccc34)c3cccc4c3c3ccccc3n4-c3ccc(-c4cccc5ccccc45)cc3)cc2)cc1]

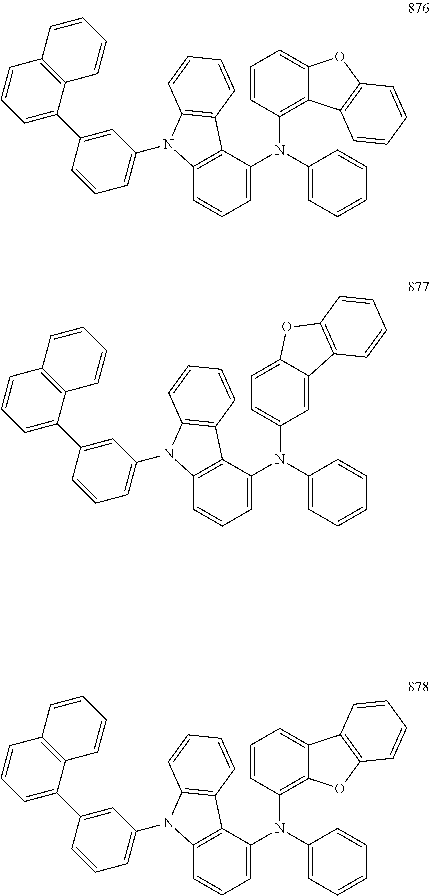 [SMILES: c1ccc(N(c2cccc3oc4ccccc4c23)c2cccc3c2c2ccccc2n3-c2cccc(-c3cccc4ccccc34)c2)cc1 ; c1ccc(N(c2ccc3oc4ccccc4c3c2)c2cccc3c2c2ccccc2n3-c2cccc(-c3cccc4ccccc34)c2)cc1 ; c1ccc(N(c2cccc3c2oc2ccccc23)c2cccc3c2c2ccccc2n3-c2cccc(-c3cccc4ccccc34)c2)cc1]

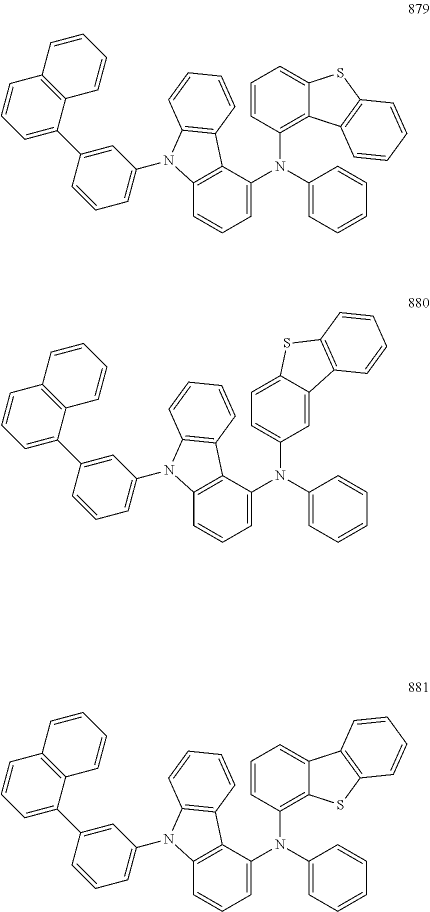 [SMILES: c1ccc(N(c2cccc3sc4ccccc4c23)c2cccc3c2c2ccccc2n3-c2cccc(-c3cccc4ccccc34)c2)cc1 ; c1ccc(N(c2ccc3sc4ccccc4c3c2)c2cccc3c2c2ccccc2n3-c2cccc(-c3cccc4ccccc34)c2)cc1 ; c1ccc(N(c2cccc3c2sc2ccccc23)c2cccc3c2c2ccccc2n3-c2cccc(-c3cccc4ccccc34)c2)cc1]

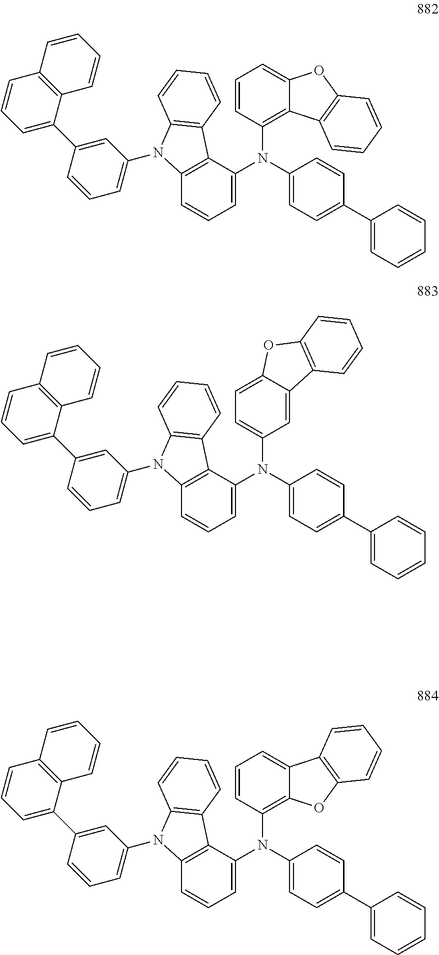 [SMILES: c1ccc(-c2ccc(N(c3cccc4oc5ccccc5c34)c3cccc4c3c3ccccc3n4-c3cccc(-c4cccc5ccccc45)c3)cc2)cc1 ; c1ccc(-c2ccc(N(c3ccc4oc5ccccc5c4c3)c3cccc4c3c3ccccc3n4-c3cccc(-c4cccc5ccccc45)c3)cc2)cc1 ; c1ccc(-c2ccc(N(c3cccc4c3oc3ccccc34)c3cccc4c3c3ccccc3n4-c3cccc(-c4cccc5ccccc45)c3)cc2)cc1]

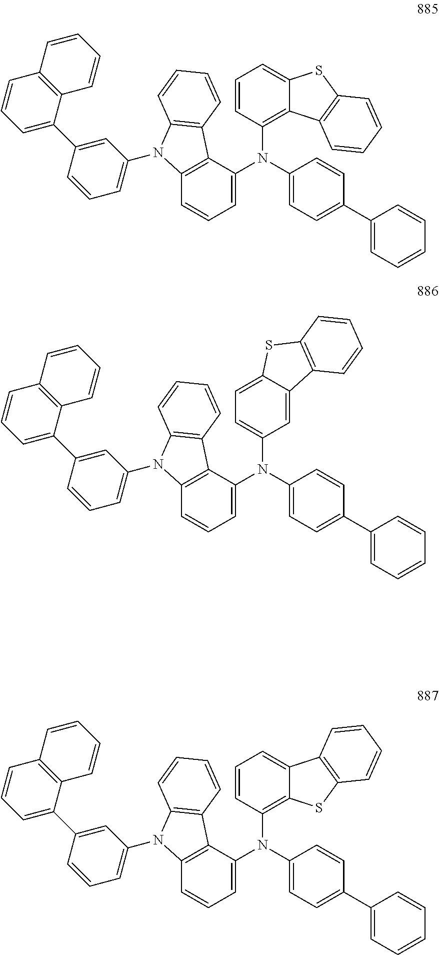 [SMILES: c1ccc(-c2ccc(N(c3cccc4sc5ccccc5c34)c3cccc4c3c3ccccc3n4-c3cccc(-c4cccc5ccccc45)c3)cc2)cc1 ; c1ccc(-c2ccc(N(c3ccc4sc5ccccc5c4c3)c3cccc4c3c3ccccc3n4-c3cccc(-c4cccc5ccccc45)c3)cc2)cc1 ; c1ccc(-c2ccc(N(c3cccc4c3sc3ccccc34)c3cccc4c3c3ccccc3n4-c3cccc(-c4cccc5ccccc45)c3)cc2)cc1]

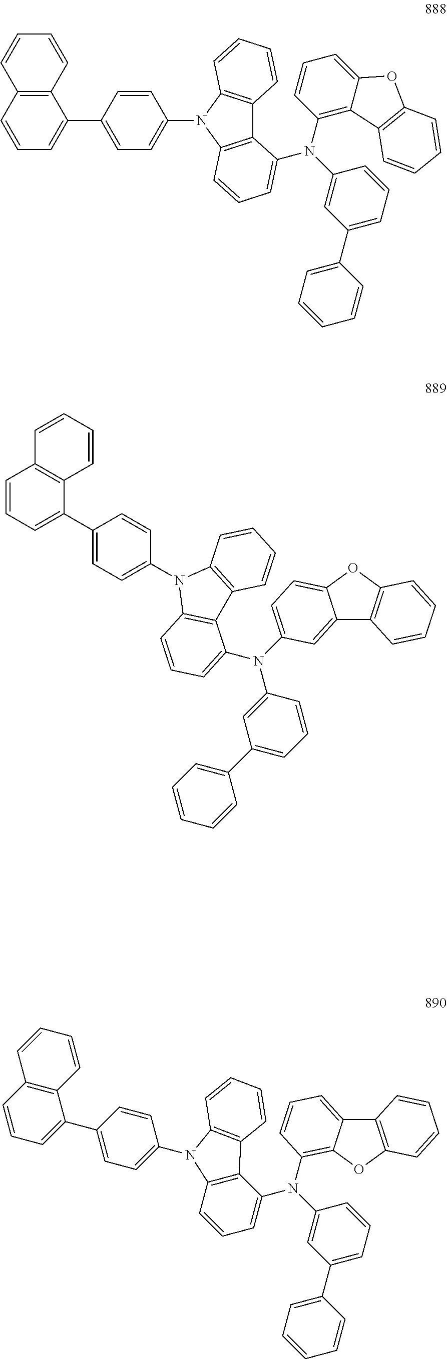 [SMILES: c1ccc(-c2cccc(N(c3cccc4oc5ccccc5c34)c3cccc4c3c3ccccc3n4-c3ccc(-c4cccc5ccccc45)cc3)c2)cc1 ; c1ccc(-c2cccc(N(c3ccc4oc5ccccc5c4c3)c3cccc4c3c3ccccc3n4-c3ccc(-c4cccc5ccccc45)cc3)c2)cc1 ; c1ccc(-c2cccc(N(c3cccc4c3oc3ccccc34)c3cccc4c3c3ccccc3n4-c3ccc(-c4cccc5ccccc45)cc3)c2)cc1]

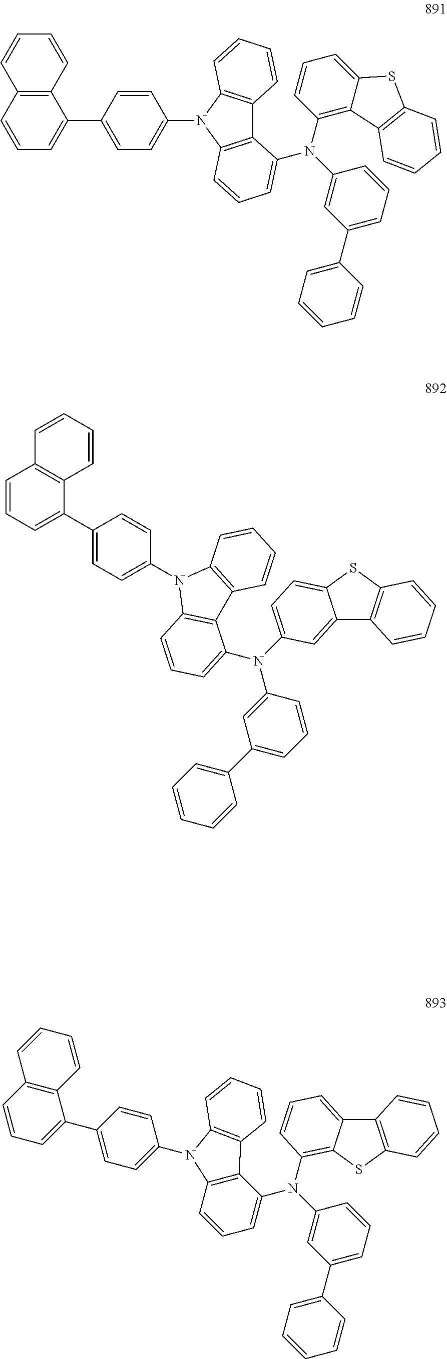 [SMILES: c1ccc(-c2cccc(N(c3cccc4sc5ccccc5c34)c3cccc4c3c3ccccc3n4-c3ccc(-c4cccc5ccccc45)cc3)c2)cc1 ; c1ccc(-c2cccc(N(c3ccc4sc5ccccc5c4c3)c3cccc4c3c3ccccc3n4-c3ccc(-c4cccc5ccccc45)cc3)c2)cc1 ; c1ccc(-c2cccc(N(c3cccc4c3sc3ccccc34)c3cccc4c3c3ccccc3n4-c3ccc(-c4cccc5ccccc45)cc3)c2)cc1]

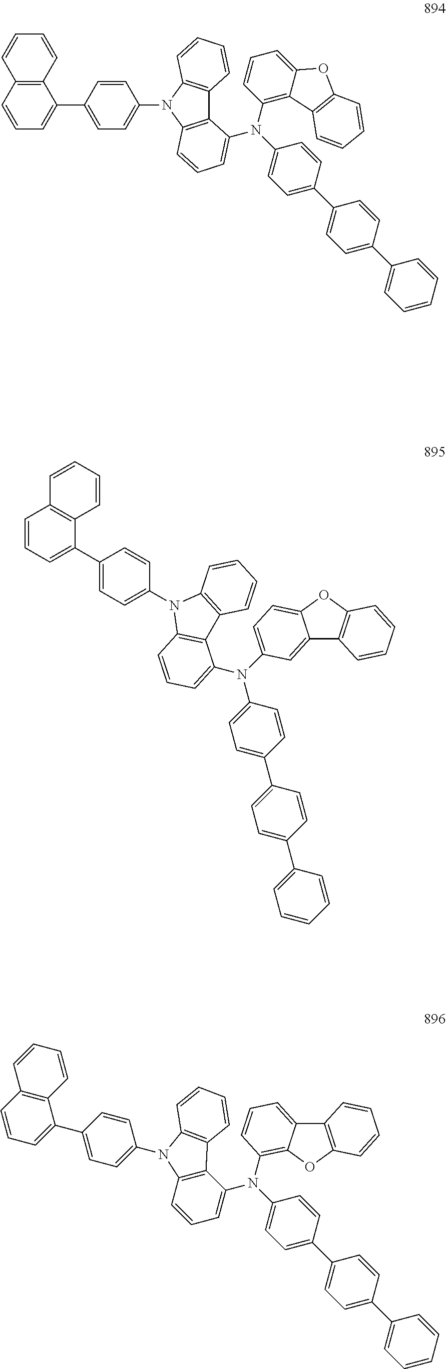 [SMILES: c1ccc(-c2ccc(-c3ccc(N(c4cccc5oc6ccccc6c45)c4cccc5c4c4ccccc4n5-c4ccc(-c5cccc6ccccc56)cc4)cc3)cc2)cc1 ; c1ccc(-c2ccc(-c3ccc(N(c4ccc5oc6ccccc6c5c4)c4cccc5c4c4ccccc4n5-c4ccc(-c5cccc6ccccc56)cc4)cc3)cc2)cc1 ; c1ccc(-c2ccc(-c3ccc(N(c4cccc5c4oc4ccccc45)c4cccc5c4c4ccccc4n5-c4ccc(-c5cccc6ccccc56)cc4)cc3)cc2)cc1]

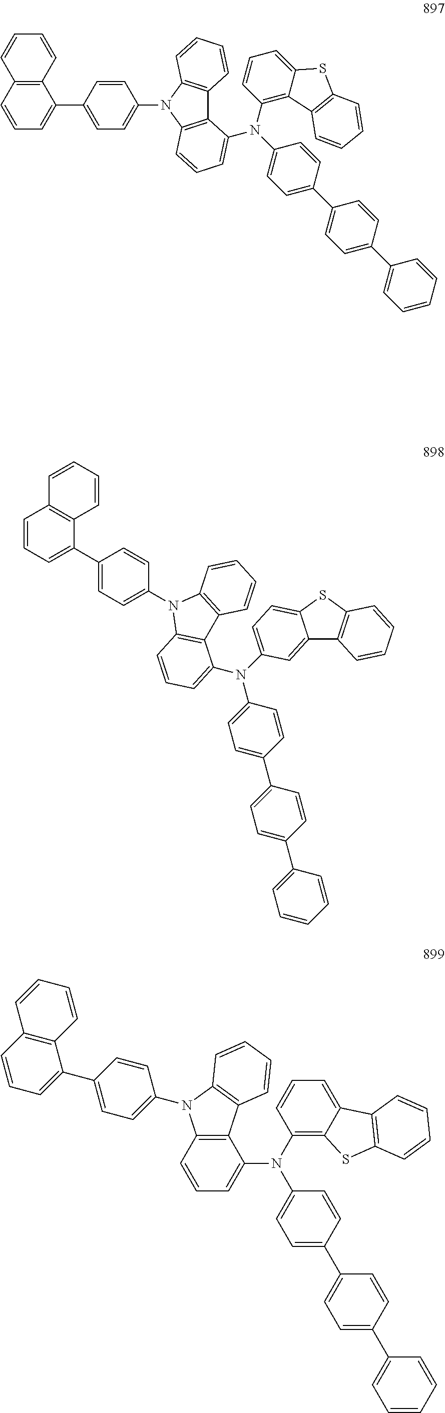 [SMILES: c1ccc(-c2ccc(-c3ccc(N(c4cccc5sc6ccccc6c45)c4cccc5c4c4ccccc4n5-c4ccc(-c5cccc6ccccc56)cc4)cc3)cc2)cc1 ; c1ccc(-c2ccc(-c3ccc(N(c4ccc5sc6ccccc6c5c4)c4cccc5c4c4ccccc4n5-c4ccc(-c5cccc6ccccc56)cc4)cc3)cc2)cc1 ; c1ccc(-c2ccc(-c3ccc(N(c4cccc5c4sc4ccccc45)c4cccc5c4c4ccccc4n5-c4ccc(-c5cccc6ccccc56)cc4)cc3)cc2)cc1]

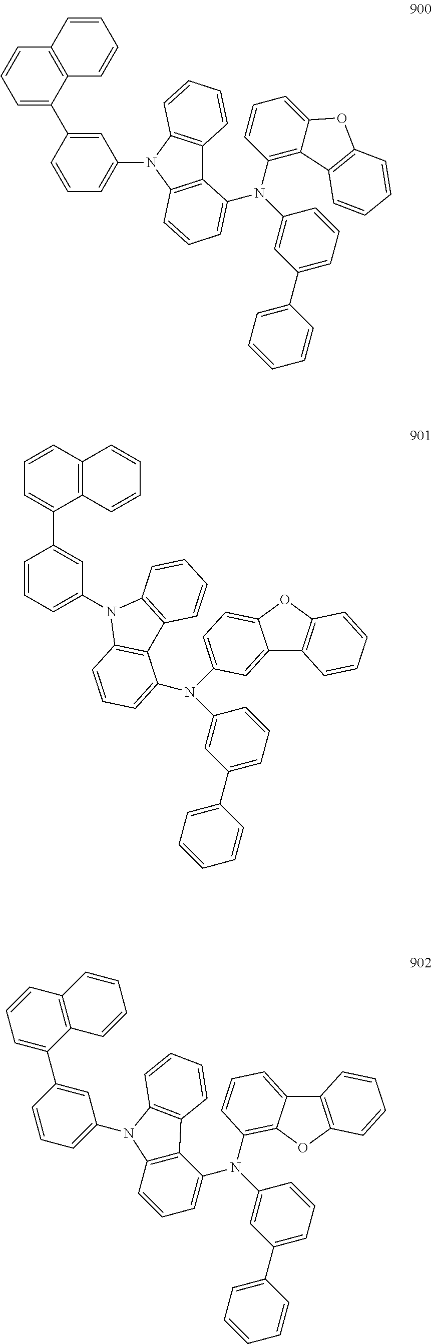 [SMILES: c1ccc(-c2cccc(N(c3cccc4oc5ccccc5c34)c3cccc4c3c3ccccc3n4-c3cccc(-c4cccc5ccccc45)c3)c2)cc1 ; c1ccc(-c2cccc(N(c3ccc4oc5ccccc5c4c3)c3cccc4c3c3ccccc3n4-c3cccc(-c4cccc5ccccc45)c3)c2)cc1 ; c1ccc(-c2cccc(N(c3cccc4c3oc3ccccc34)c3cccc4c3c3ccccc3n4-c3cccc(-c4cccc5ccccc45)c3)c2)cc1]

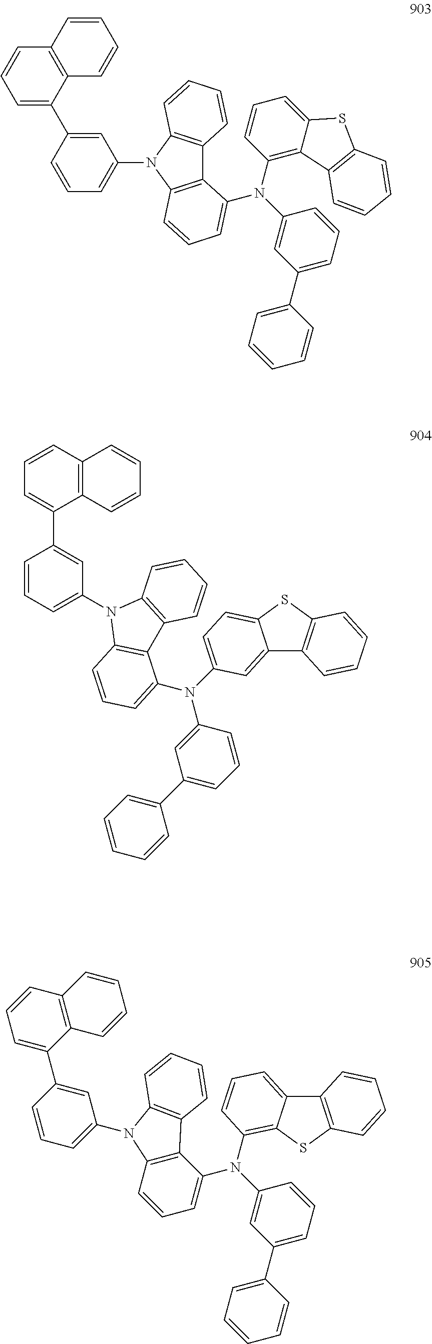 [SMILES: c1ccc(-c2cccc(N(c3cccc4sc5ccccc5c34)c3cccc4c3c3ccccc3n4-c3cccc(-c4cccc5ccccc45)c3)c2)cc1 ; c1ccc(-c2cccc(N(c3ccc4sc5ccccc5c4c3)c3cccc4c3c3ccccc3n4-c3cccc(-c4cccc5ccccc45)c3)c2)cc1 ; c1ccc(-c2cccc(N(c3cccc4c3sc3ccccc34)c3cccc4c3c3ccccc3n4-c3cccc(-c4cccc5ccccc45)c3)c2)cc1]

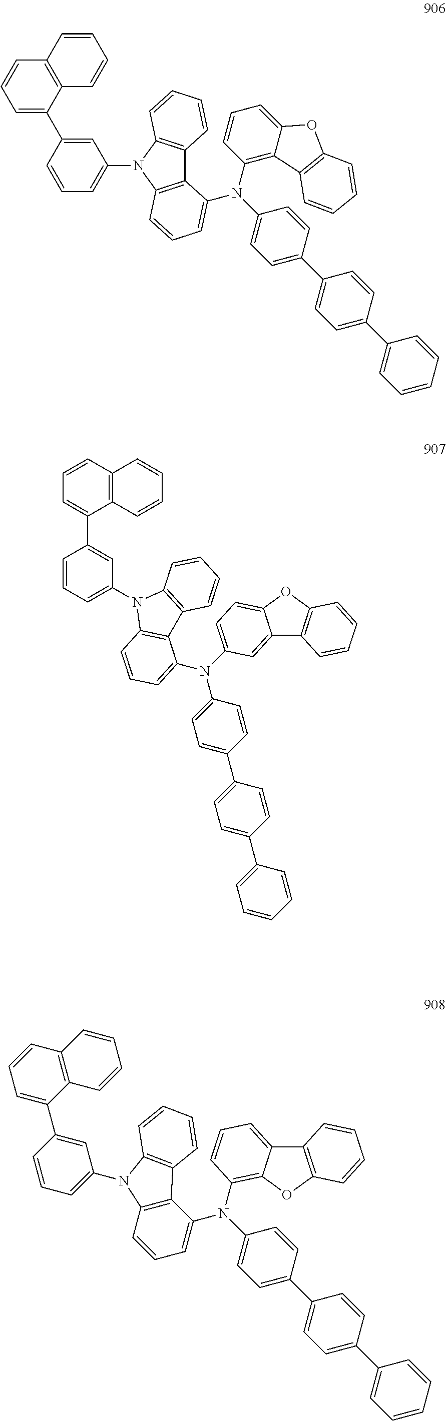 [SMILES: c1ccc(-c2ccc(-c3ccc(N(c4cccc5oc6ccccc6c45)c4cccc5c4c4ccccc4n5-c4cccc(-c5cccc6ccccc56)c4)cc3)cc2)cc1 ; c1ccc(-c2ccc(-c3ccc(N(c4ccc5oc6ccccc6c5c4)c4cccc5c4c4ccccc4n5-c4cccc(-c5cccc6ccccc56)c4)cc3)cc2)cc1 ; c1ccc(-c2ccc(-c3ccc(N(c4cccc5c4oc4ccccc45)c4cccc5c4c4ccccc4n5-c4cccc(-c5cccc6ccccc56)c4)cc3)cc2)cc1]

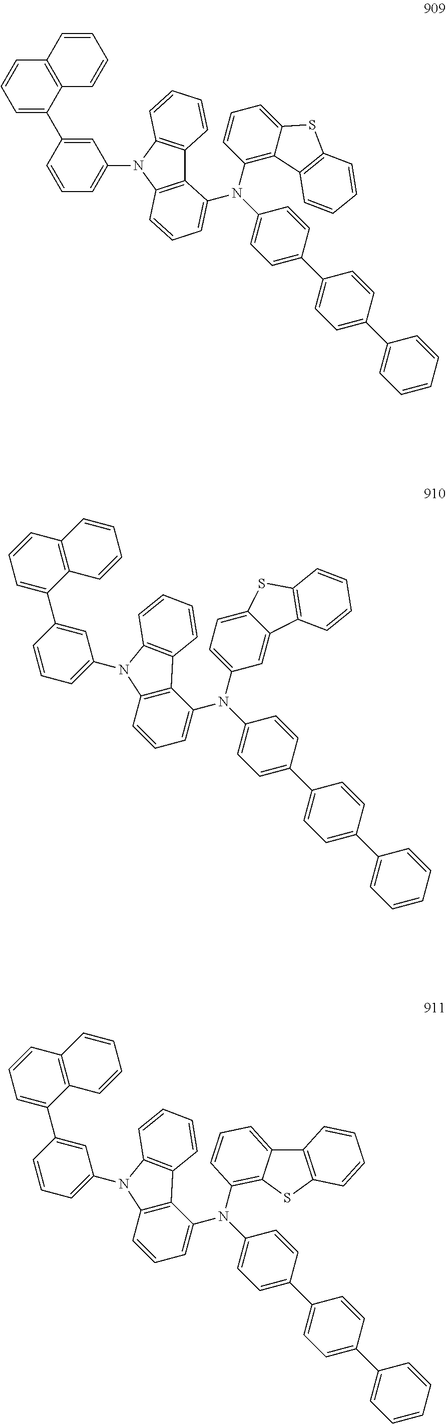 [SMILES: c1ccc(-c2ccc(-c3ccc(N(c4cccc5sc6ccccc6c45)c4cccc5c4c4ccccc4n5-c4cccc(-c5cccc6ccccc56)c4)cc3)cc2)cc1 ; c1ccc(-c2ccc(-c3ccc(N(c4ccc5sc6ccccc6c5c4)c4cccc5c4c4ccccc4n5-c4cccc(-c5cccc6ccccc56)c4)cc3)cc2)cc1 ; c1ccc(-c2ccc(-c3ccc(N(c4cccc5c4sc4ccccc45)c4cccc5c4c4ccccc4n5-c4cccc(-c5cccc6ccccc56)c4)cc3)cc2)cc1]

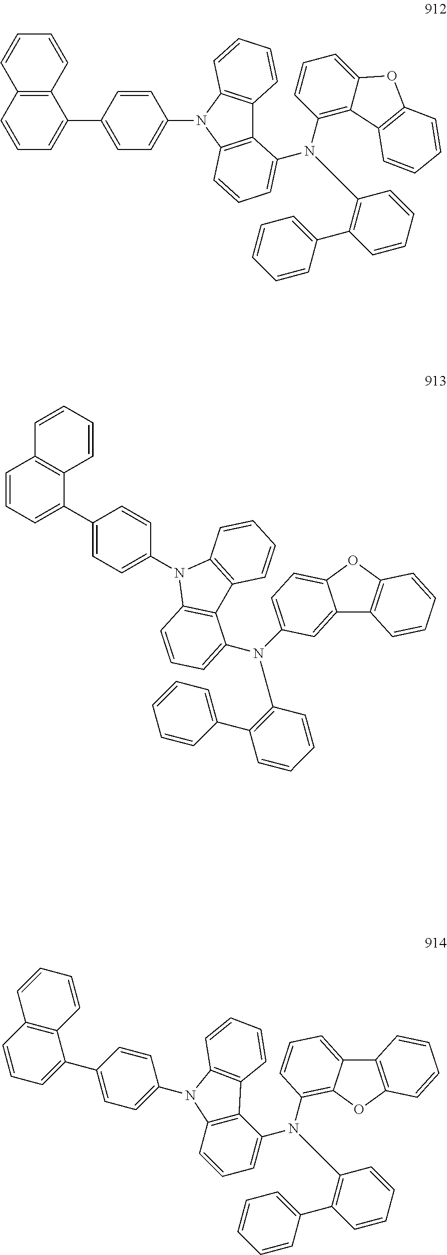 [SMILES: c1ccc(-c2ccccc2N(c2cccc3oc4ccccc4c23)c2cccc3c2c2ccccc2n3-c2ccc(-c3cccc4ccccc34)cc2)cc1 ; c1ccc(-c2ccccc2N(c2ccc3oc4ccccc4c3c2)c2cccc3c2c2ccccc2n3-c2ccc(-c3cccc4ccccc34)cc2)cc1 ; c1ccc(-c2ccccc2N(c2cccc3c2oc2ccccc23)c2cccc3c2c2ccccc2n3-c2ccc(-c3cccc4ccccc34)cc2)cc1]

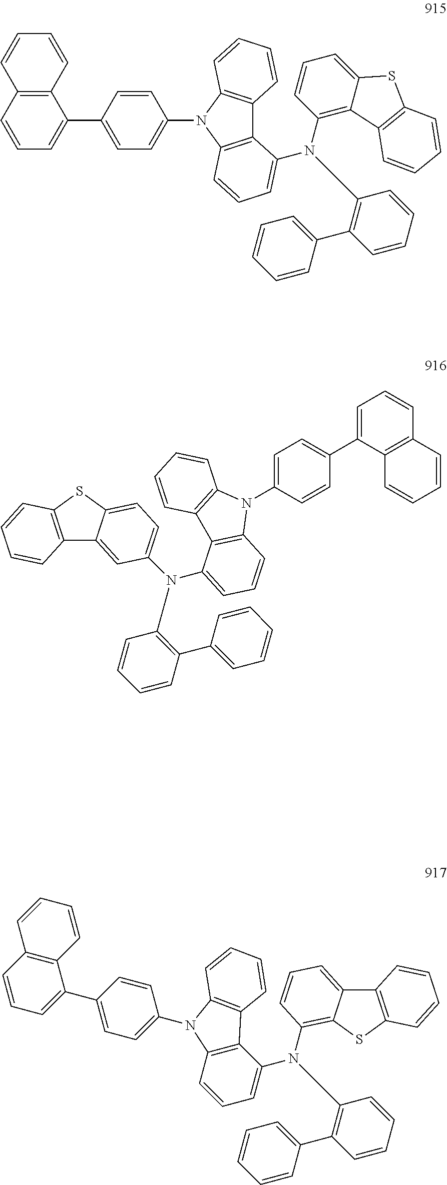 [SMILES: c1ccc(-c2ccccc2N(c2cccc3sc4ccccc4c23)c2cccc3c2c2ccccc2n3-c2ccc(-c3cccc4ccccc34)cc2)cc1 ; c1ccc(-c2ccccc2N(c2ccc3sc4ccccc4c3c2)c2cccc3c2c2ccccc2n3-c2ccc(-c3cccc4ccccc34)cc2)cc1 ; c1ccc(-c2ccccc2N(c2cccc3c2sc2ccccc23)c2cccc3c2c2ccccc2n3-c2ccc(-c3cccc4ccccc34)cc2)cc1]

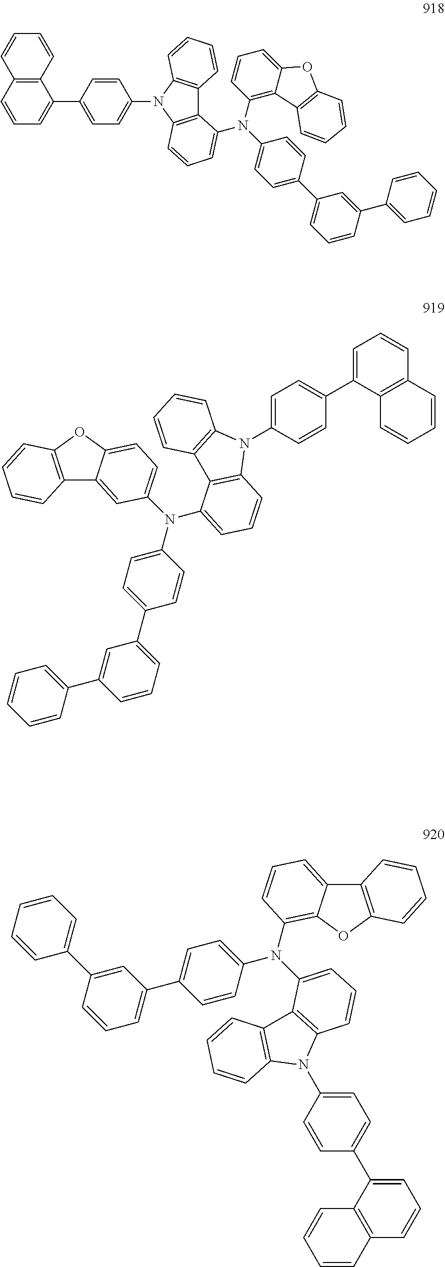 [SMILES: c1ccc(-c2cccc(-c3ccc(N(c4cccc5oc6ccccc6c45)c4cccc5c4c4ccccc4n5-c4ccc(-c5cccc6ccccc56)cc4)cc3)c2)cc1 ; c1ccc(-c2cccc(-c3ccc(N(c4ccc5oc6ccccc6c5c4)c4cccc5c4c4ccccc4n5-c4ccc(-c5cccc6ccccc56)cc4)cc3)c2)cc1 ; c1ccc(-c2cccc(-c3ccc(N(c4cccc5c4oc4ccccc45)c4cccc5c4c4ccccc4n5-c4ccc(-c5cccc6ccccc56)cc4)cc3)c2)cc1]

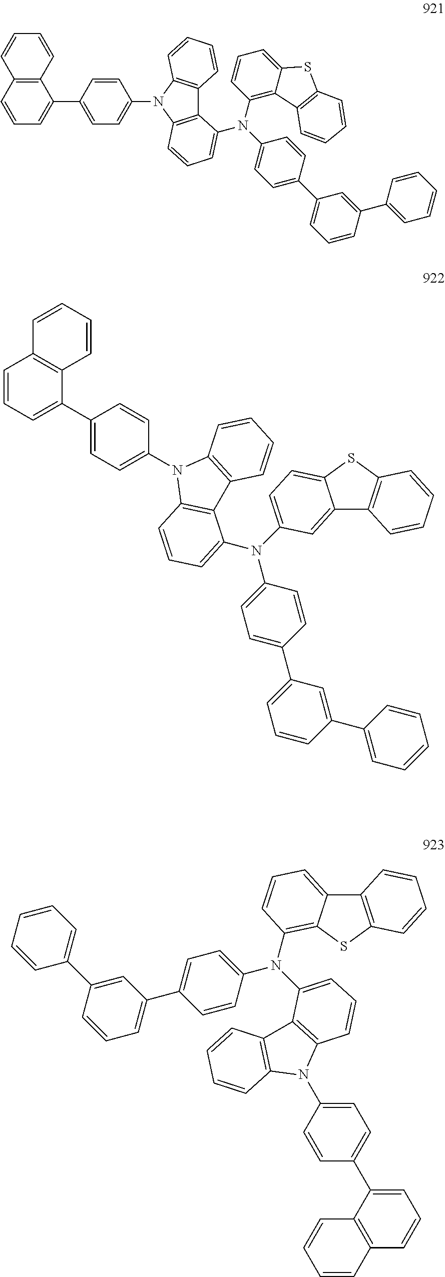 [SMILES: c1ccc(-c2cccc(-c3ccc(N(c4cccc5sc6ccccc6c45)c4cccc5c4c4ccccc4n5-c4ccc(-c5cccc6ccccc56)cc4)cc3)c2)cc1 ; c1ccc(-c2cccc(-c3ccc(N(c4ccc5sc6ccccc6c5c4)c4cccc5c4c4ccccc4n5-c4ccc(-c5cccc6ccccc56)cc4)cc3)c2)cc1 ; c1ccc(-c2cccc(-c3ccc(N(c4cccc5c4sc4ccccc45)c4cccc5c4c4ccccc4n5-c4ccc(-c5cccc6ccccc56)cc4)cc3)c2)cc1]

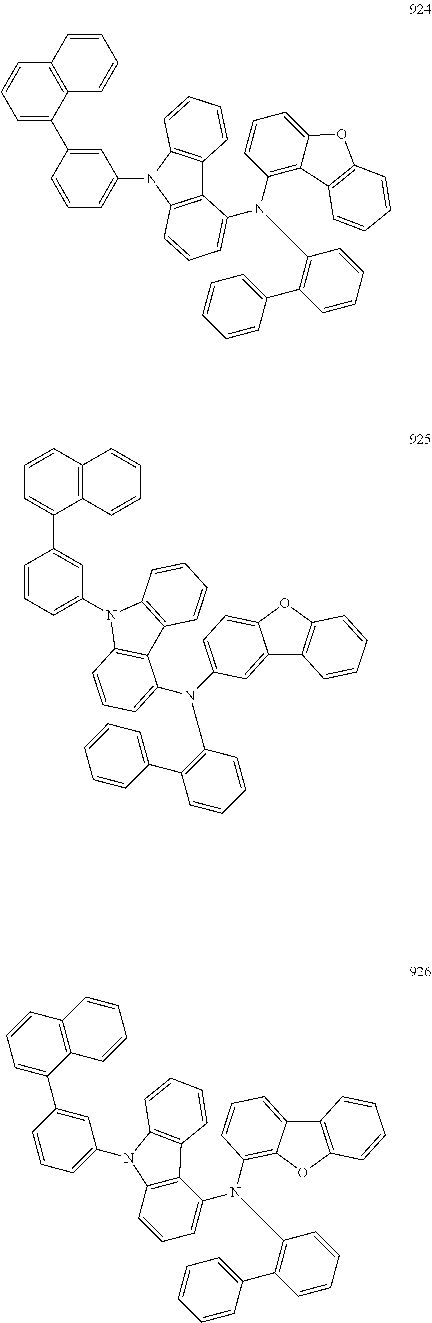 [SMILES: c1ccc(-c2ccccc2N(c2cccc3oc4ccccc4c23)c2cccc3c2c2ccccc2n3-c2cccc(-c3cccc4ccccc34)c2)cc1 ; c1ccc(-c2ccccc2N(c2ccc3oc4ccccc4c3c2)c2cccc3c2c2ccccc2n3-c2cccc(-c3cccc4ccccc34)c2)cc1 ; c1ccc(-c2ccccc2N(c2cccc3c2oc2ccccc23)c2cccc3c2c2ccccc2n3-c2cccc(-c3cccc4ccccc34)c2)cc1]

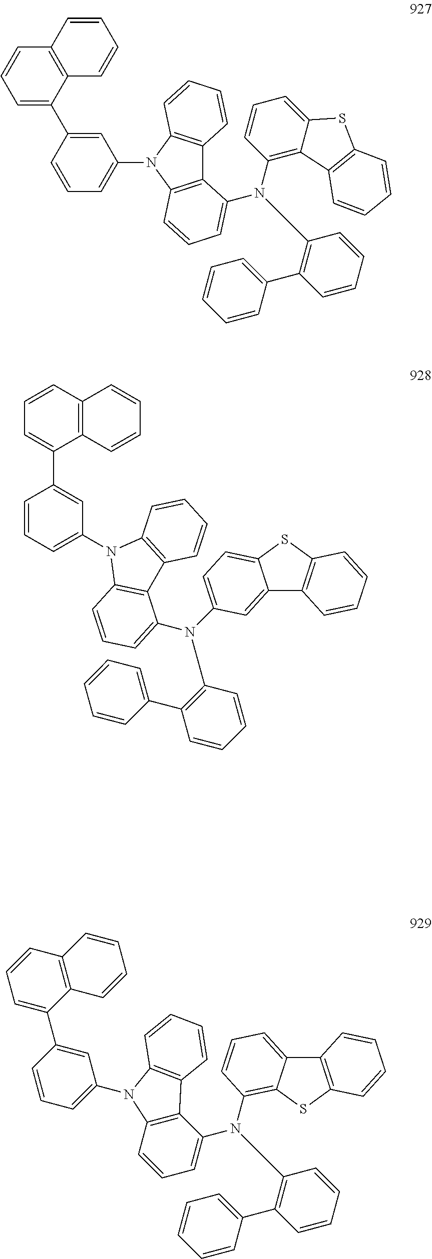 [SMILES: c1ccc(-c2ccccc2N(c2cccc3sc4ccccc4c23)c2cccc3c2c2ccccc2n3-c2cccc(-c3cccc4ccccc34)c2)cc1 ; c1ccc(-c2ccccc2N(c2ccc3sc4ccccc4c3c2)c2cccc3c2c2ccccc2n3-c2cccc(-c3cccc4ccccc34)c2)cc1 ; c1ccc(-c2ccccc2N(c2cccc3c2sc2ccccc23)c2cccc3c2c2ccccc2n3-c2cccc(-c3cccc4ccccc34)c2)cc1]

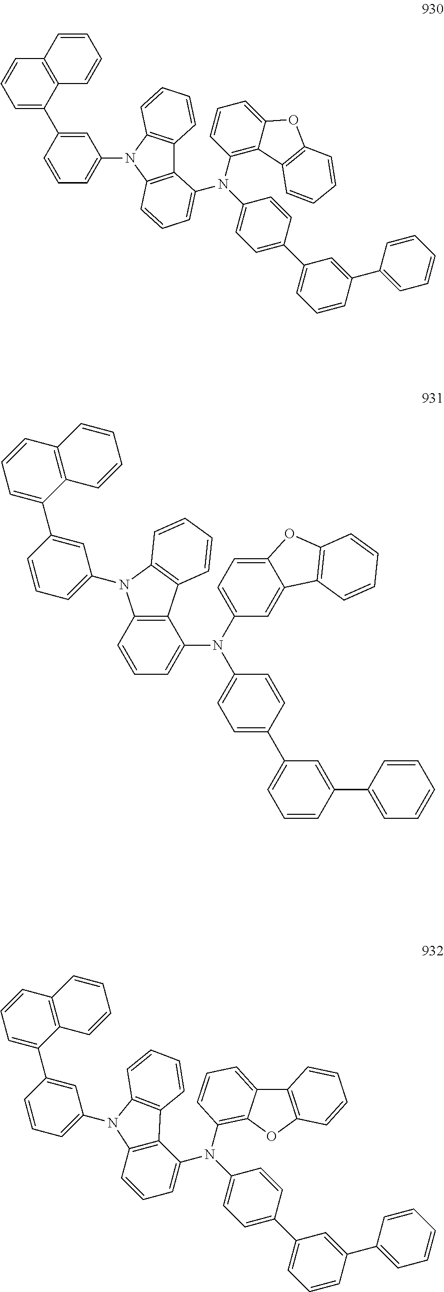 [SMILES: c1ccc(-c2cccc(-c3ccc(N(c4cccc5oc6ccccc6c45)c4cccc5c4c4ccccc4n5-c4cccc(-c5cccc6ccccc56)c4)cc3)c2)cc1 ; c1ccc(-c2cccc(-c3ccc(N(c4ccc5oc6ccccc6c5c4)c4cccc5c4c4ccccc4n5-c4cccc(-c5cccc6ccccc56)c4)cc3)c2)cc1 ; c1ccc(-c2cccc(-c3ccc(N(c4cccc5c4oc4ccccc45)c4cccc5c4c4ccccc4n5-c4cccc(-c5cccc6ccccc56)c4)cc3)c2)cc1]

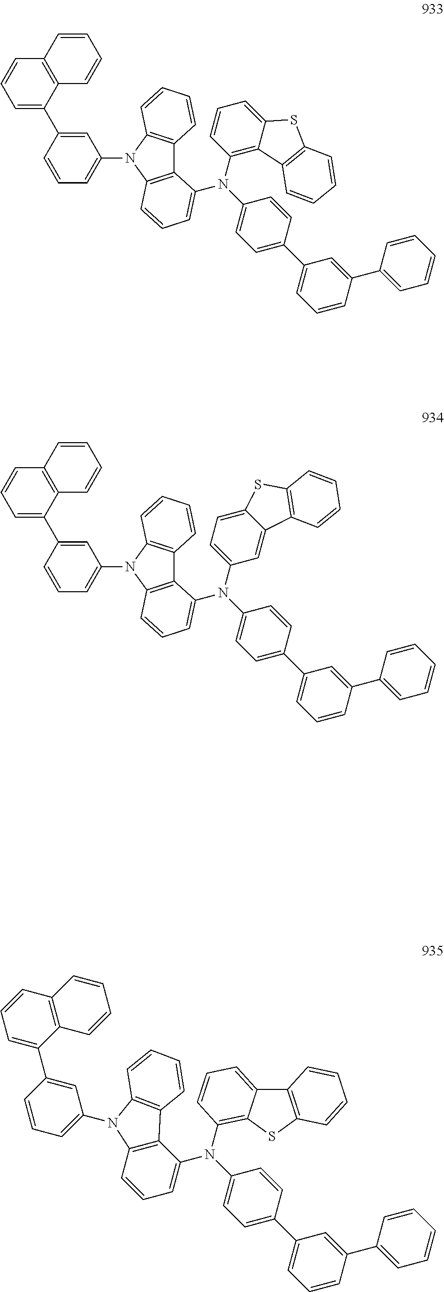 [SMILES: c1ccc(-c2cccc(-c3ccc(N(c4cccc5sc6ccccc6c45)c4cccc5c4c4ccccc4n5-c4cccc(-c5cccc6ccccc56)c4)cc3)c2)cc1 ; c1ccc(-c2cccc(-c3ccc(N(c4ccc5sc6ccccc6c5c4)c4cccc5c4c4ccccc4n5-c4cccc(-c5cccc6ccccc56)c4)cc3)c2)cc1 ; c1ccc(-c2cccc(-c3ccc(N(c4cccc5c4sc4ccccc45)c4cccc5c4c4ccccc4n5-c4cccc(-c5cccc6ccccc56)c4)cc3)c2)cc1]

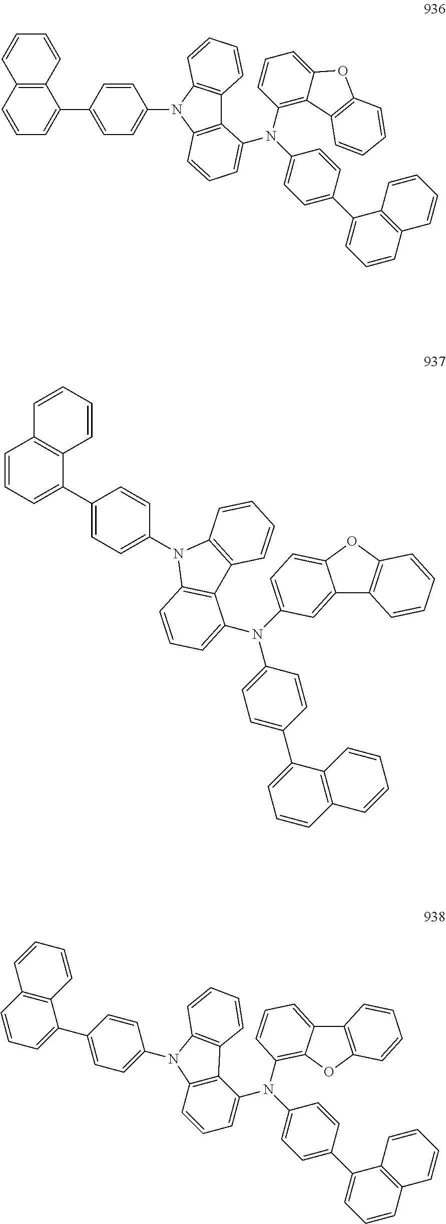 [SMILES: c1ccc2c(-c3ccc(N(c4cccc5oc6ccccc6c45)c4cccc5c4c4ccccc4n5-c4ccc(-c5cccc6ccccc56)cc4)cc3)cccc2c1 ; c1ccc2c(-c3ccc(N(c4ccc5oc6ccccc6c5c4)c4cccc5c4c4ccccc4n5-c4ccc(-c5cccc6ccccc56)cc4)cc3)cccc2c1 ; c1ccc2c(-c3ccc(N(c4cccc5c4oc4ccccc45)c4cccc5c4c4ccccc4n5-c4ccc(-c5cccc6ccccc56)cc4)cc3)cccc2c1]

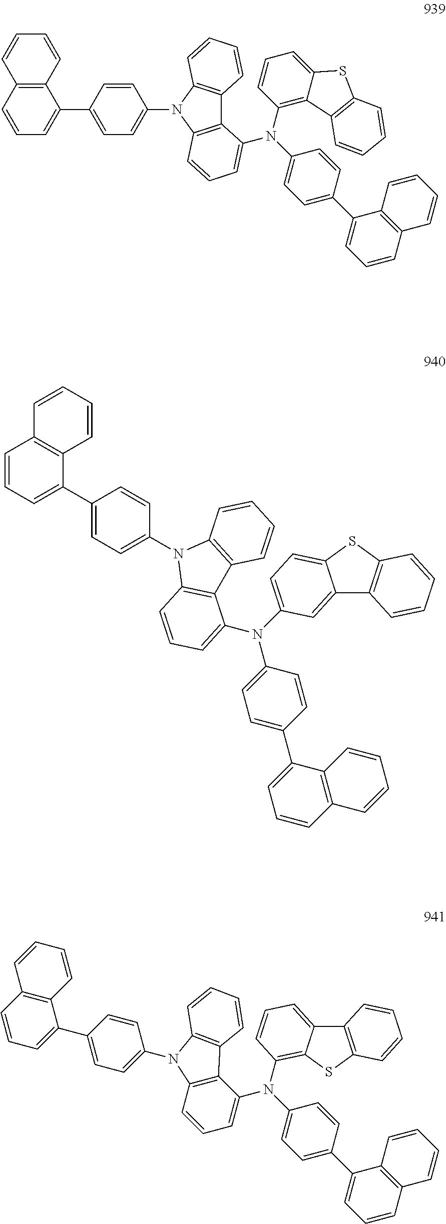 [SMILES: c1ccc2c(-c3ccc(N(c4cccc5sc6ccccc6c45)c4cccc5c4c4ccccc4n5-c4ccc(-c5cccc6ccccc56)cc4)cc3)cccc2c1 ; c1ccc2c(-c3ccc(N(c4ccc5sc6ccccc6c5c4)c4cccc5c4c4ccccc4n5-c4ccc(-c5cccc6ccccc56)cc4)cc3)cccc2c1 ; c1ccc2c(-c3ccc(N(c4cccc5c4sc4ccccc45)c4cccc5c4c4ccccc4n5-c4ccc(-c5cccc6ccccc56)cc4)cc3)cccc2c1]

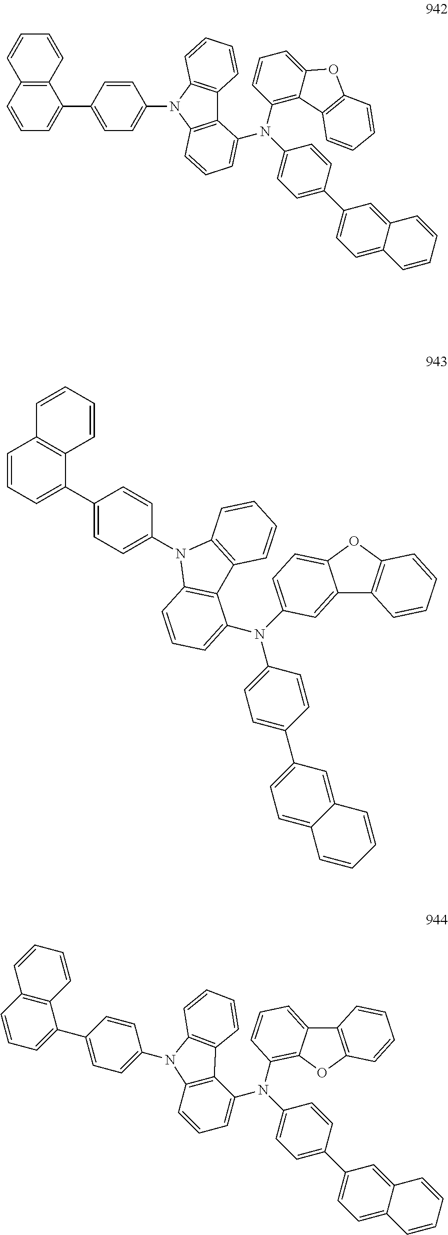 [SMILES: c1ccc2cc(-c3ccc(N(c4cccc5oc6ccccc6c45)c4cccc5c4c4ccccc4n5-c4ccc(-c5cccc6ccccc56)cc4)cc3)ccc2c1 ; c1ccc2cc(-c3ccc(N(c4ccc5oc6ccccc6c5c4)c4cccc5c4c4ccccc4n5-c4ccc(-c5cccc6ccccc56)cc4)cc3)ccc2c1 ; c1ccc2cc(-c3ccc(N(c4cccc5c4oc4ccccc45)c4cccc5c4c4ccccc4n5-c4ccc(-c5cccc6ccccc56)cc4)cc3)ccc2c1]

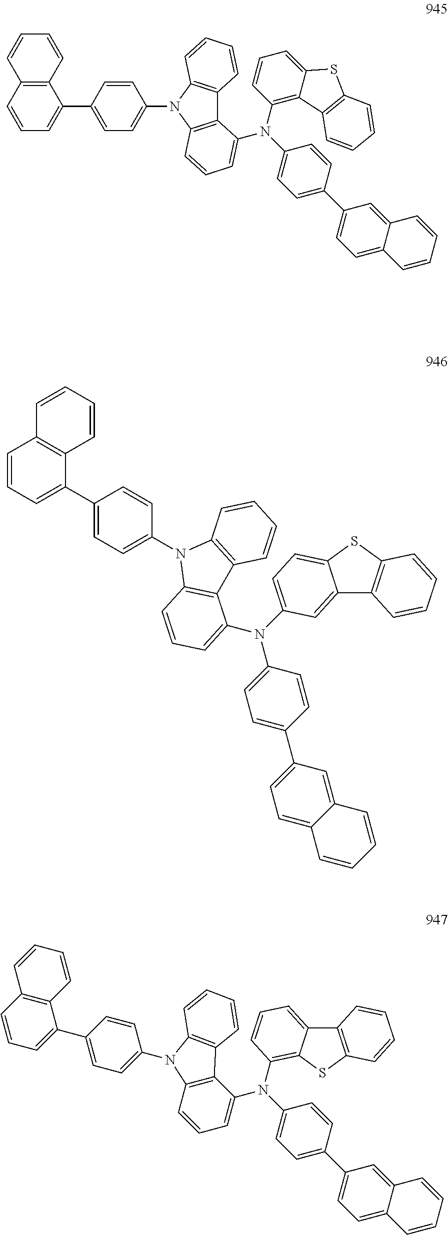 [SMILES: c1ccc2cc(-c3ccc(N(c4cccc5sc6ccccc6c45)c4cccc5c4c4ccccc4n5-c4ccc(-c5cccc6ccccc56)cc4)cc3)ccc2c1 ; c1ccc2cc(-c3ccc(N(c4ccc5sc6ccccc6c5c4)c4cccc5c4c4ccccc4n5-c4ccc(-c5cccc6ccccc56)cc4)cc3)ccc2c1 ; c1ccc2cc(-c3ccc(N(c4cccc5c4sc4ccccc45)c4cccc5c4c4ccccc4n5-c4ccc(-c5cccc6ccccc56)cc4)cc3)ccc2c1]

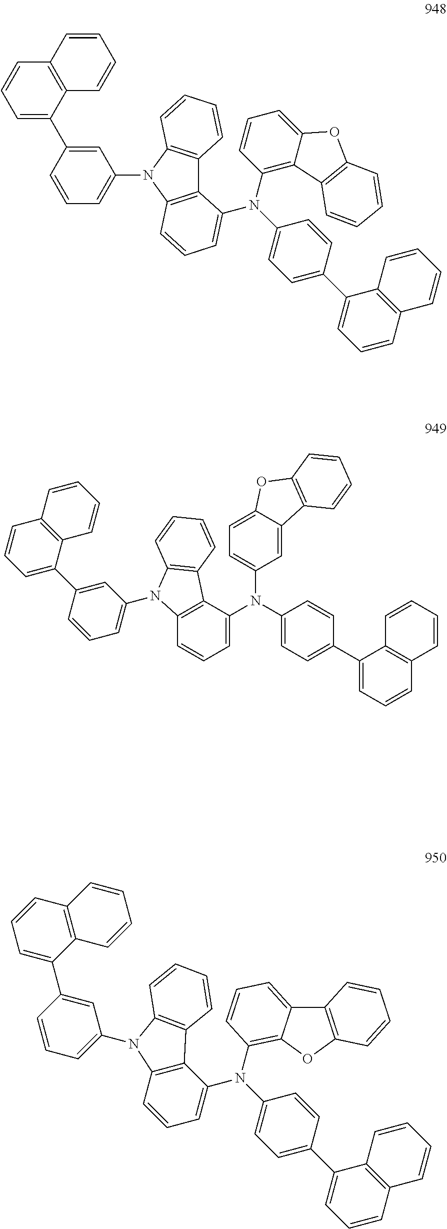 [SMILES: c1cc(-c2cccc3ccccc23)cc(-n2c3ccccc3c3c(N(c4ccc(-c5cccc6ccccc56)cc4)c4cccc5oc6ccccc6c45)cccc32)c1 ; c1cc(-c2cccc3ccccc23)cc(-n2c3ccccc3c3c(N(c4ccc(-c5cccc6ccccc56)cc4)c4ccc5oc6ccccc6c5c4)cccc32)c1 ; c1cc(-c2cccc3ccccc23)cc(-n2c3ccccc3c3c(N(c4ccc(-c5cccc6ccccc56)cc4)c4cccc5c4oc4ccccc45)cccc32)c1]

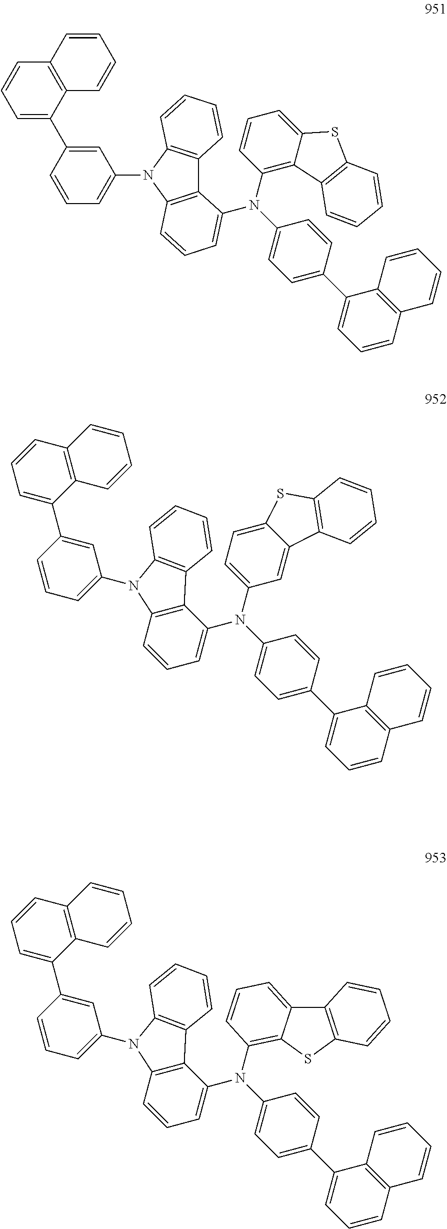 [SMILES: c1cc(-c2cccc3ccccc23)cc(-n2c3ccccc3c3c(N(c4ccc(-c5cccc6ccccc56)cc4)c4cccc5sc6ccccc6c45)cccc32)c1 ; c1cc(-c2cccc3ccccc23)cc(-n2c3ccccc3c3c(N(c4ccc(-c5cccc6ccccc56)cc4)c4ccc5sc6ccccc6c5c4)cccc32)c1 ; c1cc(-c2cccc3ccccc23)cc(-n2c3ccccc3c3c(N(c4ccc(-c5cccc6ccccc56)cc4)c4cccc5c4sc4ccccc45)cccc32)c1]

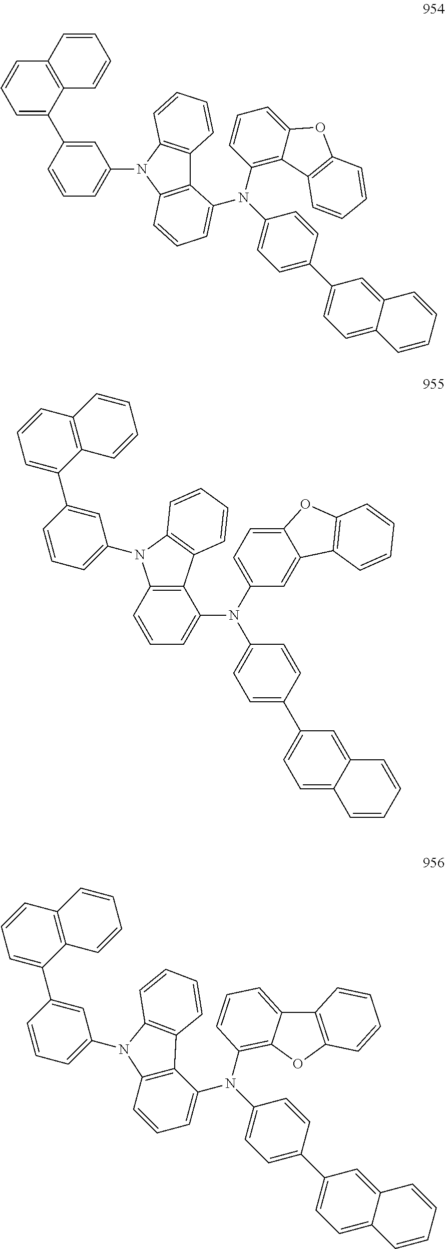 [SMILES: c1cc(-c2cccc3ccccc23)cc(-n2c3ccccc3c3c(N(c4ccc(-c5ccc6ccccc6c5)cc4)c4cccc5oc6ccccc6c45)cccc32)c1 ; c1cc(-c2cccc3ccccc23)cc(-n2c3ccccc3c3c(N(c4ccc(-c5ccc6ccccc6c5)cc4)c4ccc5oc6ccccc6c5c4)cccc32)c1 ; c1cc(-c2cccc3ccccc23)cc(-n2c3ccccc3c3c(N(c4ccc(-c5ccc6ccccc6c5)cc4)c4cccc5c4oc4ccccc45)cccc32)c1]

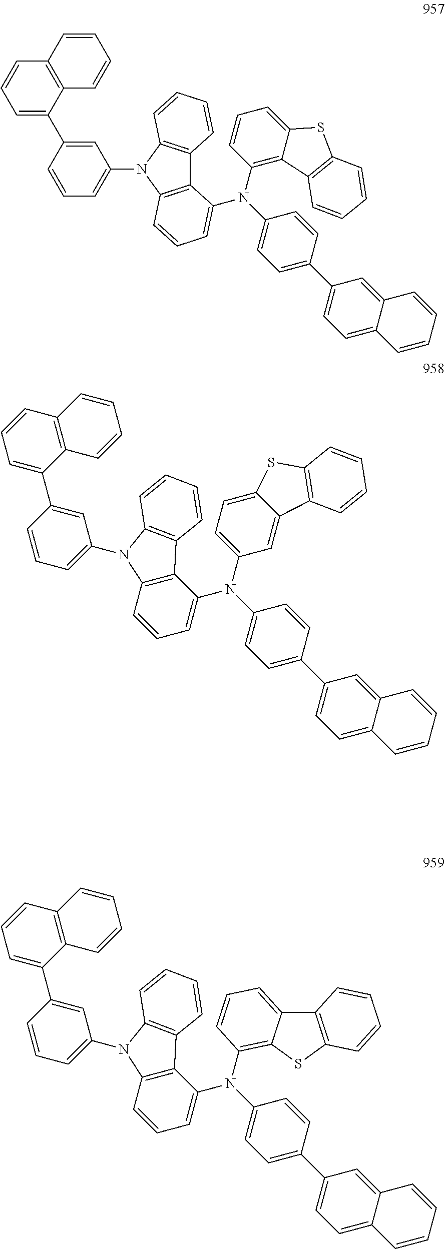 [SMILES: c1cc(-c2cccc3ccccc23)cc(-n2c3ccccc3c3c(N(c4ccc(-c5ccc6ccccc6c5)cc4)c4cccc5sc6ccccc6c45)cccc32)c1 ; c1cc(-c2cccc3ccccc23)cc(-n2c3ccccc3c3c(N(c4ccc(-c5ccc6ccccc6c5)cc4)c4ccc5sc6ccccc6c5c4)cccc32)c1 ; c1cc(-c2cccc3ccccc23)cc(-n2c3ccccc3c3c(N(c4ccc(-c5ccc6ccccc6c5)cc4)c4cccc5c4sc4ccccc45)cccc32)c1]

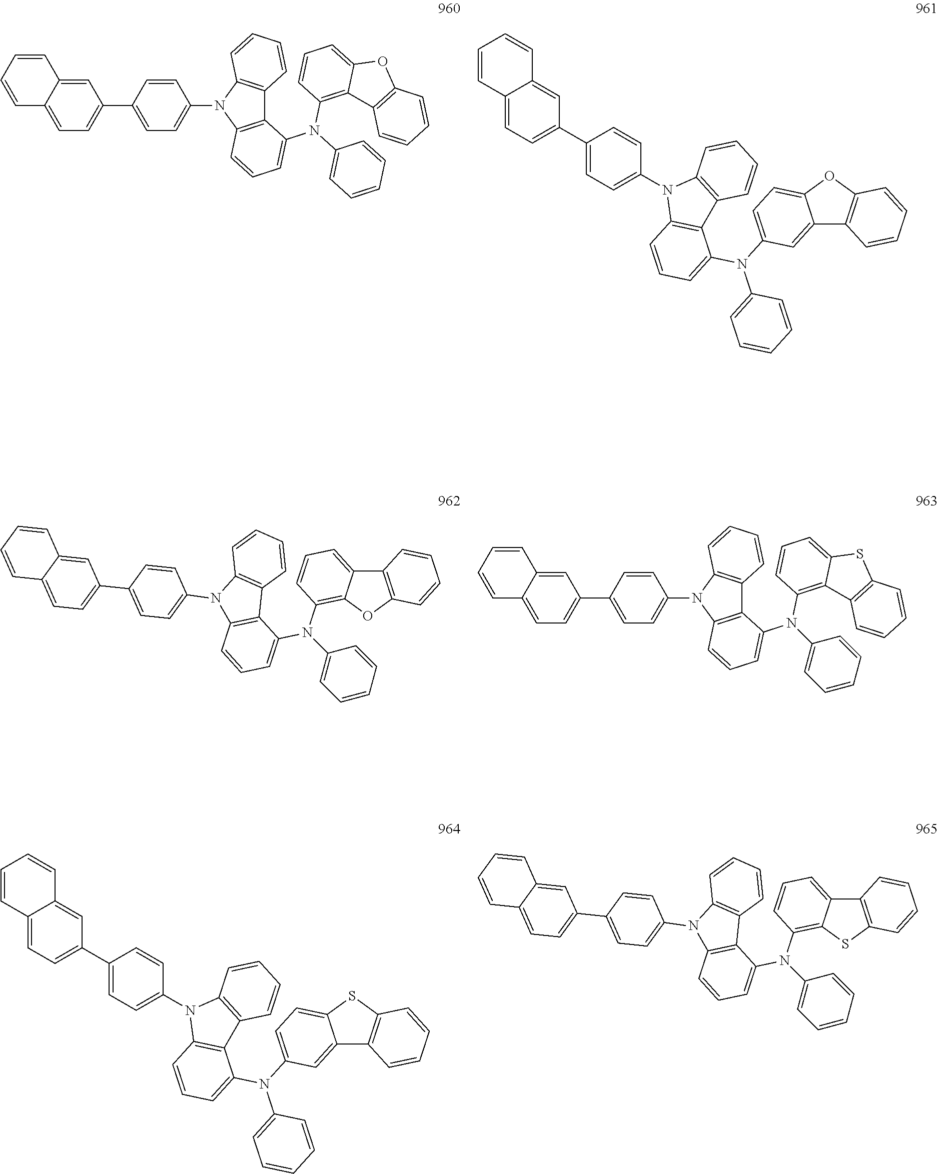

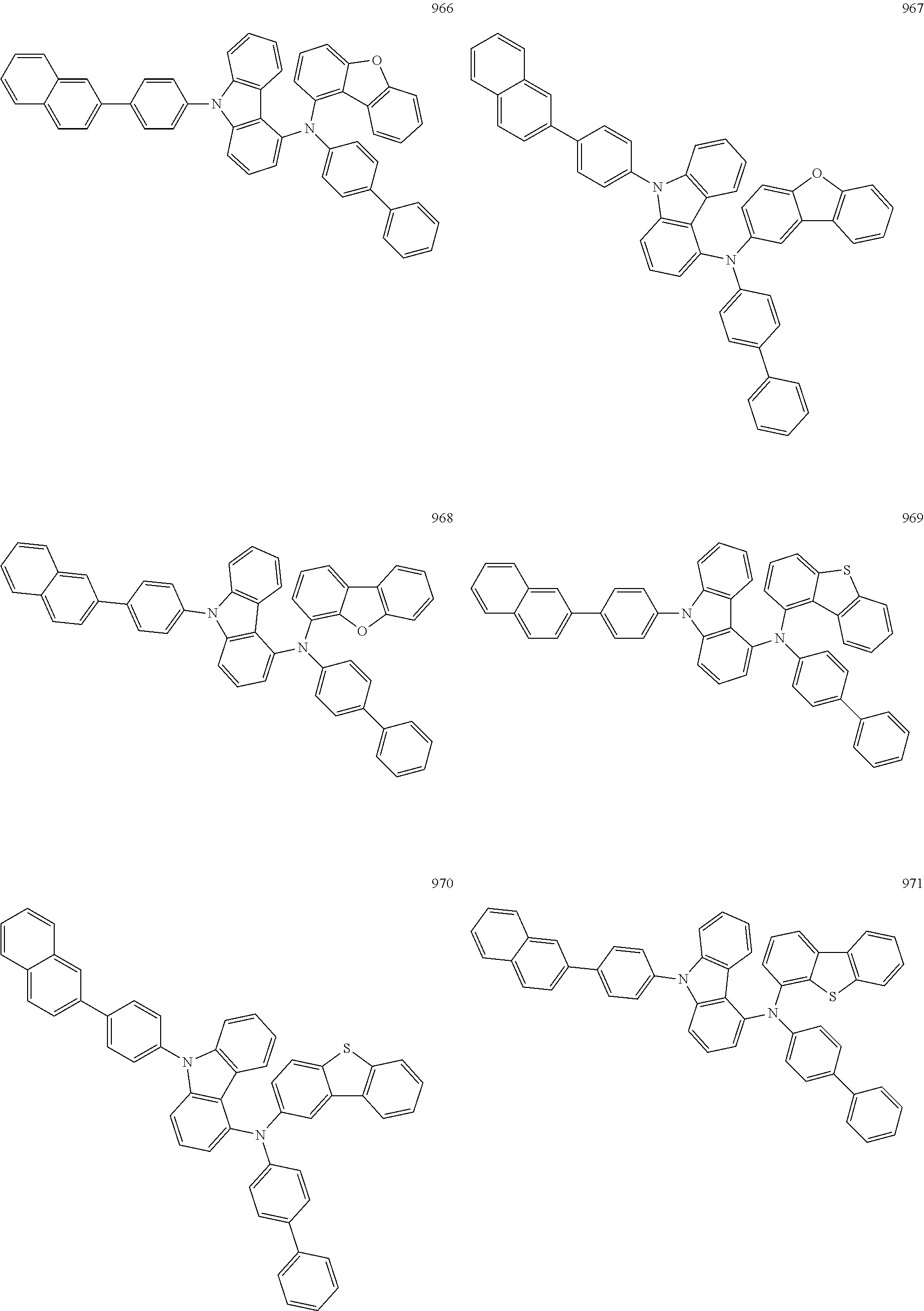

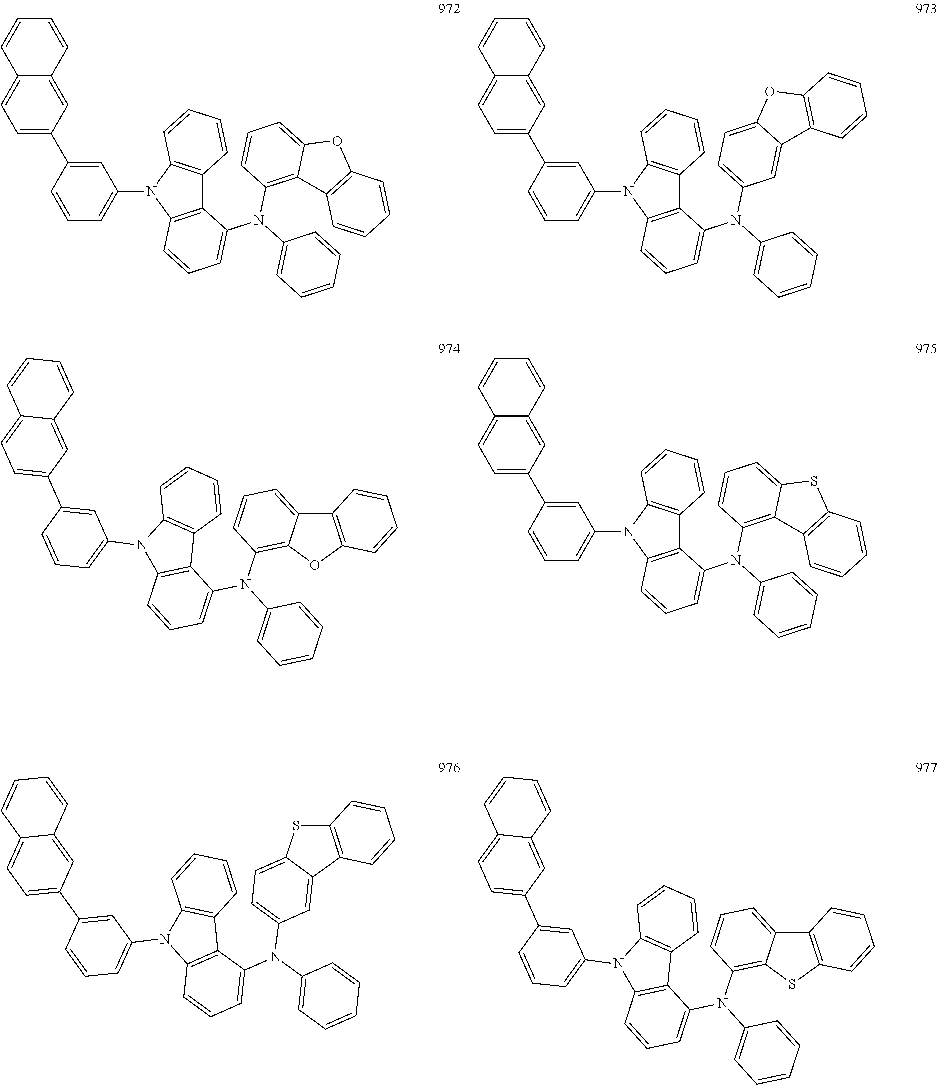 [SMILES: c1ccc(N(c2cccc3oc4ccccc4c23)c2cccc3c2c2ccccc2n3-c2cccc(-c3ccc4ccccc4c3)c2)cc1 ; c1ccc(N(c2ccc3oc4ccccc4c3c2)c2cccc3c2c2ccccc2n3-c2cccc(-c3ccc4ccccc4c3)c2)cc1 ; c1ccc(N(c2cccc3c2oc2ccccc23)c2cccc3c2c2ccccc2n3-c2cccc(-c3ccc4ccccc4c3)c2)cc1 ; c1ccc(N(c2cccc3sc4ccccc4c23)c2cccc3c2c2ccccc2n3-c2cccc(-c3ccc4ccccc4c3)c2)cc1 ; c1ccc(N(c2ccc3sc4ccccc4c3c2)c2cccc3c2c2ccccc2n3-c2cccc(-c3ccc4ccccc4c3)c2)cc1 ; c1ccc(N(c2cccc3c2sc2ccccc23)c2cccc3c2c2ccccc2n3-c2cccc(-c3ccc4ccccc4c3)c2)cc1]

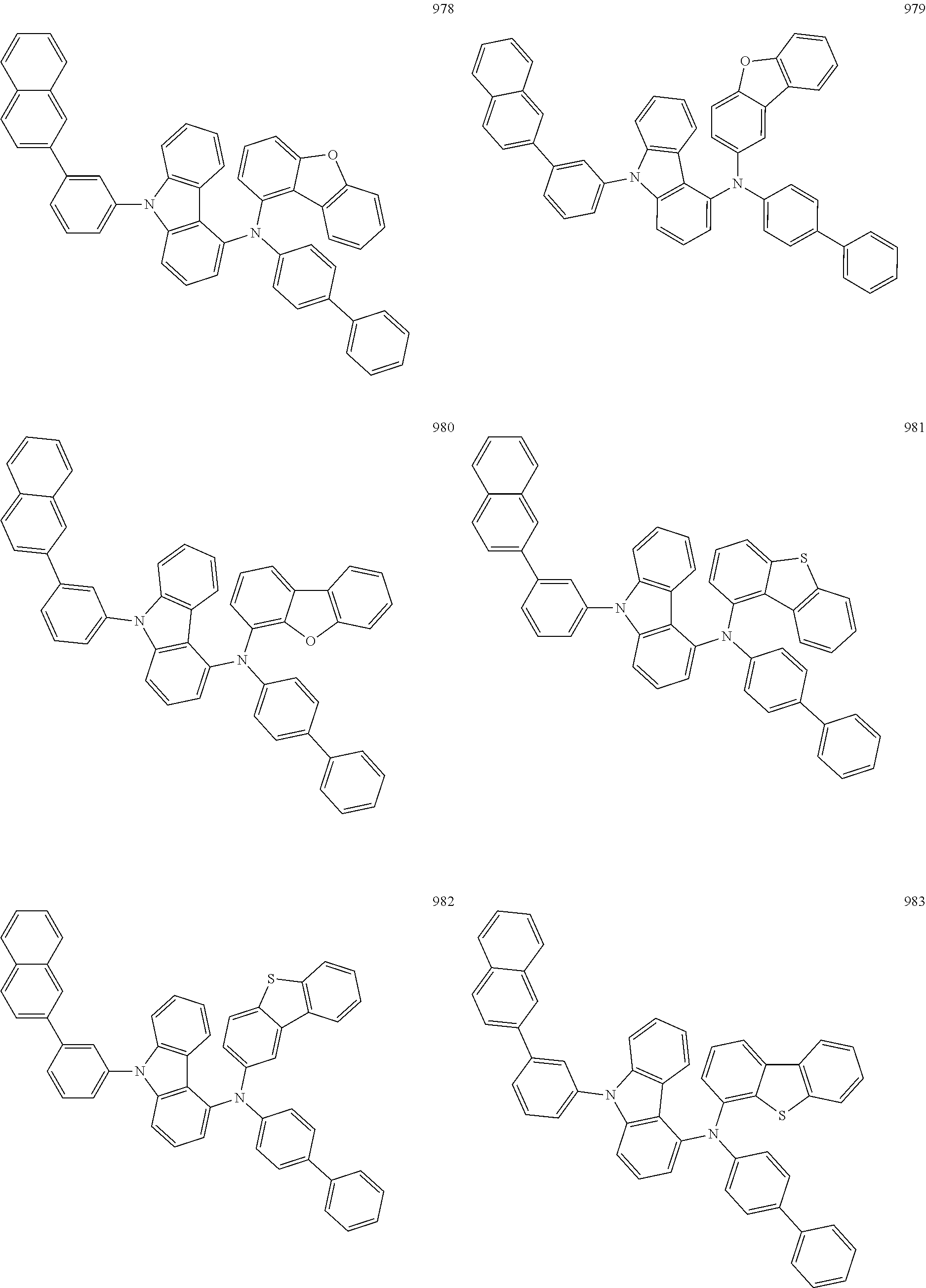

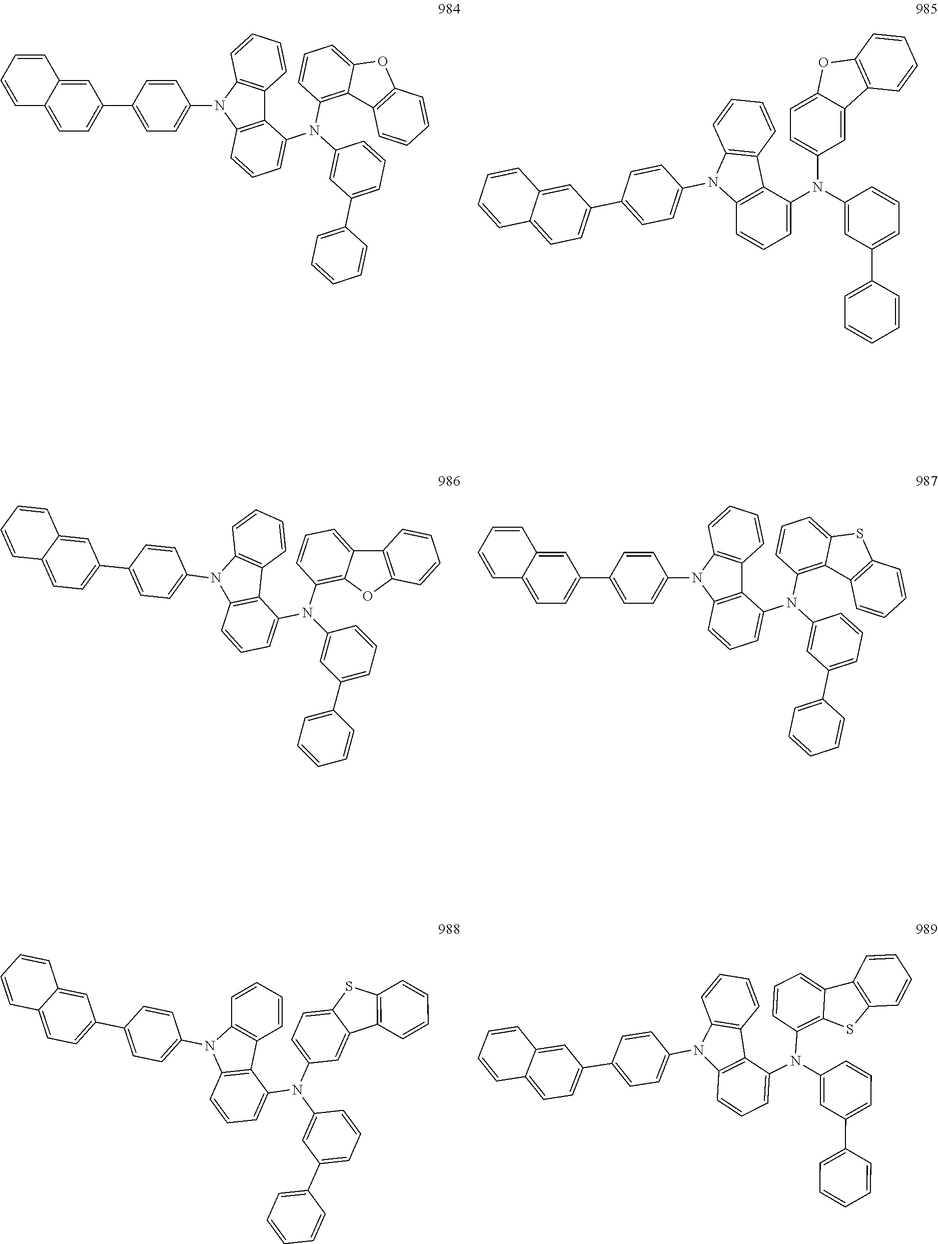

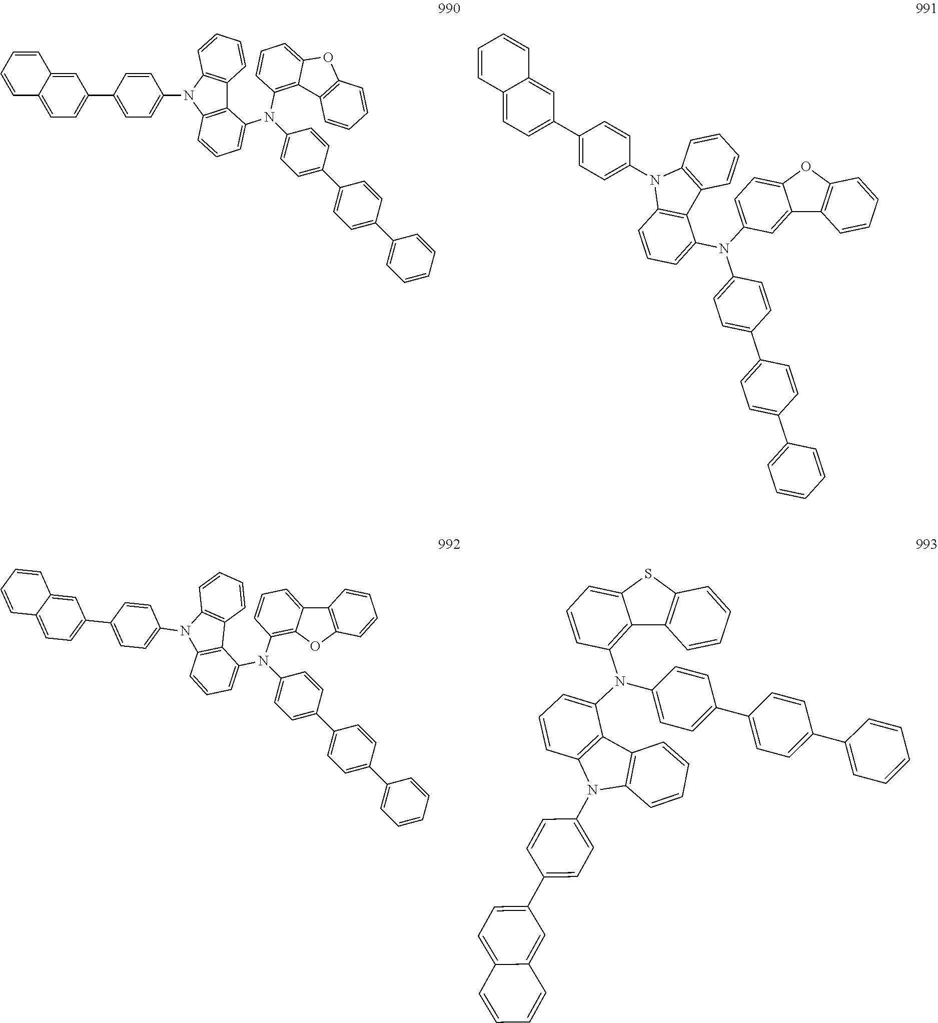 [SMILES: c1ccc(-c2ccc(-c3ccc(N(c4cccc5oc6ccccc6c45)c4cccc5c4c4ccccc4n5-c4ccc(-c5ccc6ccccc6c5)cc4)cc3)cc2)cc1 ; c1ccc(-c2ccc(-c3ccc(N(c4ccc5oc6ccccc6c5c4)c4cccc5c4c4ccccc4n5-c4ccc(-c5ccc6ccccc6c5)cc4)cc3)cc2)cc1 ; c1ccc(-c2ccc(-c3ccc(N(c4cccc5c4oc4ccccc45)c4cccc5c4c4ccccc4n5-c4ccc(-c5ccc6ccccc6c5)cc4)cc3)cc2)cc1 ; c1ccc(-c2ccc(-c3ccc(N(c4cccc5sc6ccccc6c45)c4cccc5c4c4ccccc4n5-c4ccc(-c5ccc6ccccc6c5)cc4)cc3)cc2)cc1]

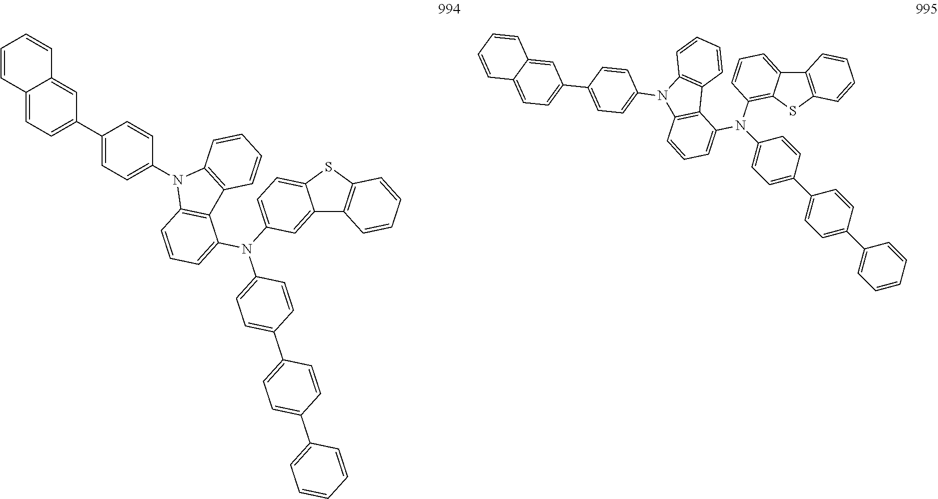 [SMILES: c1ccc(-c2ccc(-c3ccc(N(c4ccc5sc6ccccc6c5c4)c4cccc5c4c4ccccc4n5-c4ccc(-c5ccc6ccccc6c5)cc4)cc3)cc2)cc1 ; c1ccc(-c2ccc(-c3ccc(N(c4cccc5c4sc4ccccc45)c4cccc5c4c4ccccc4n5-c4ccc(-c5ccc6ccccc6c5)cc4)cc3)cc2)cc1]

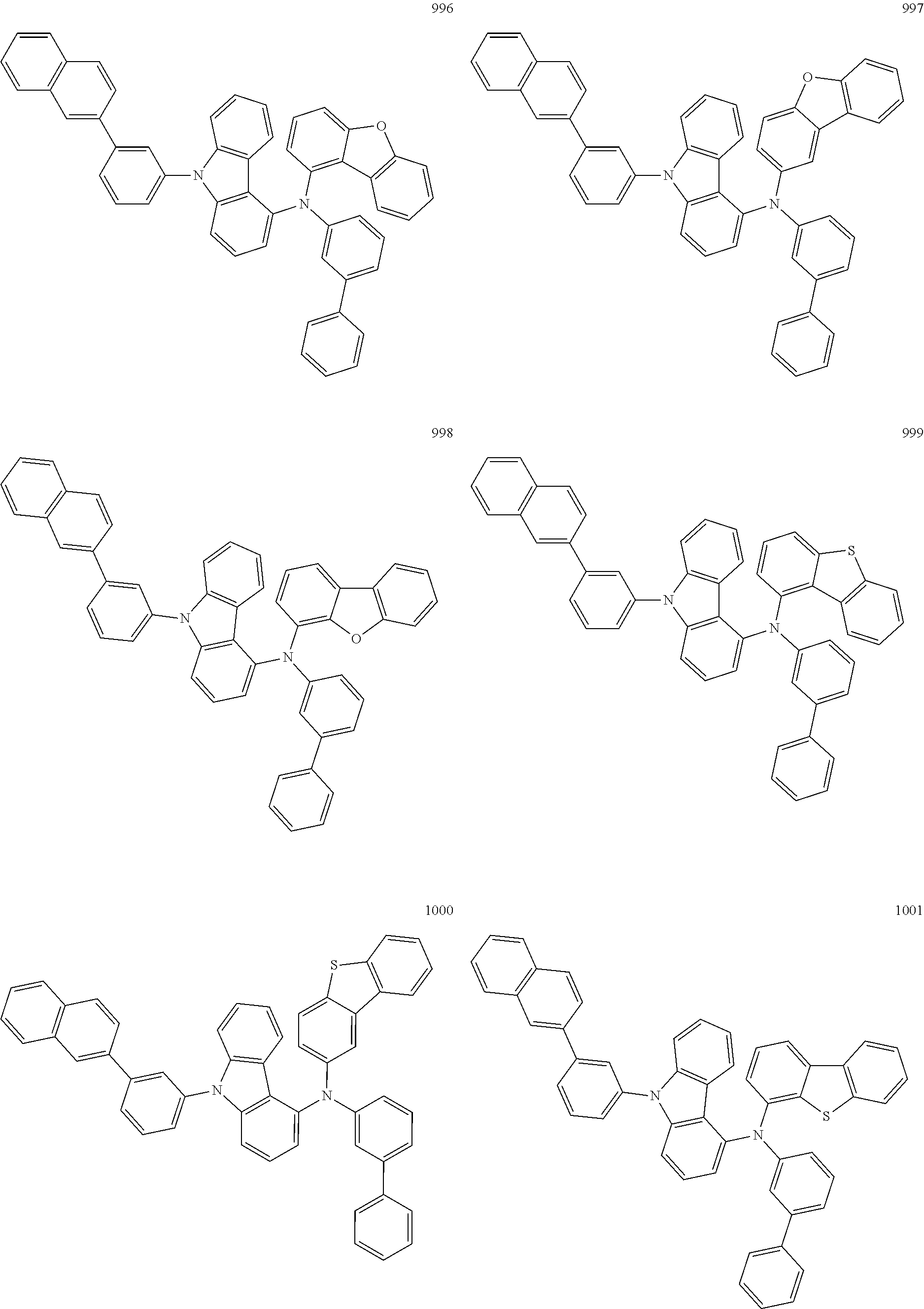

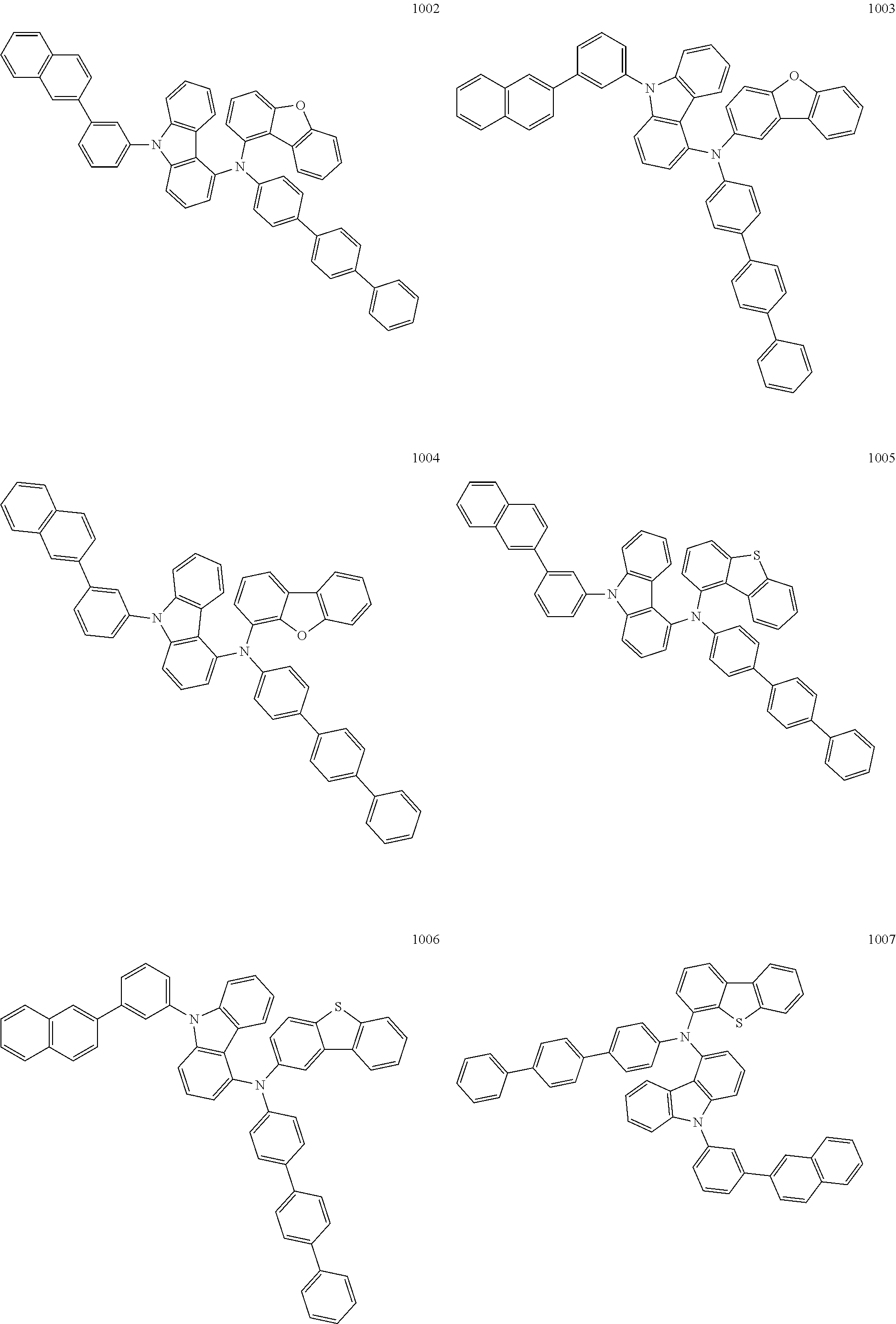

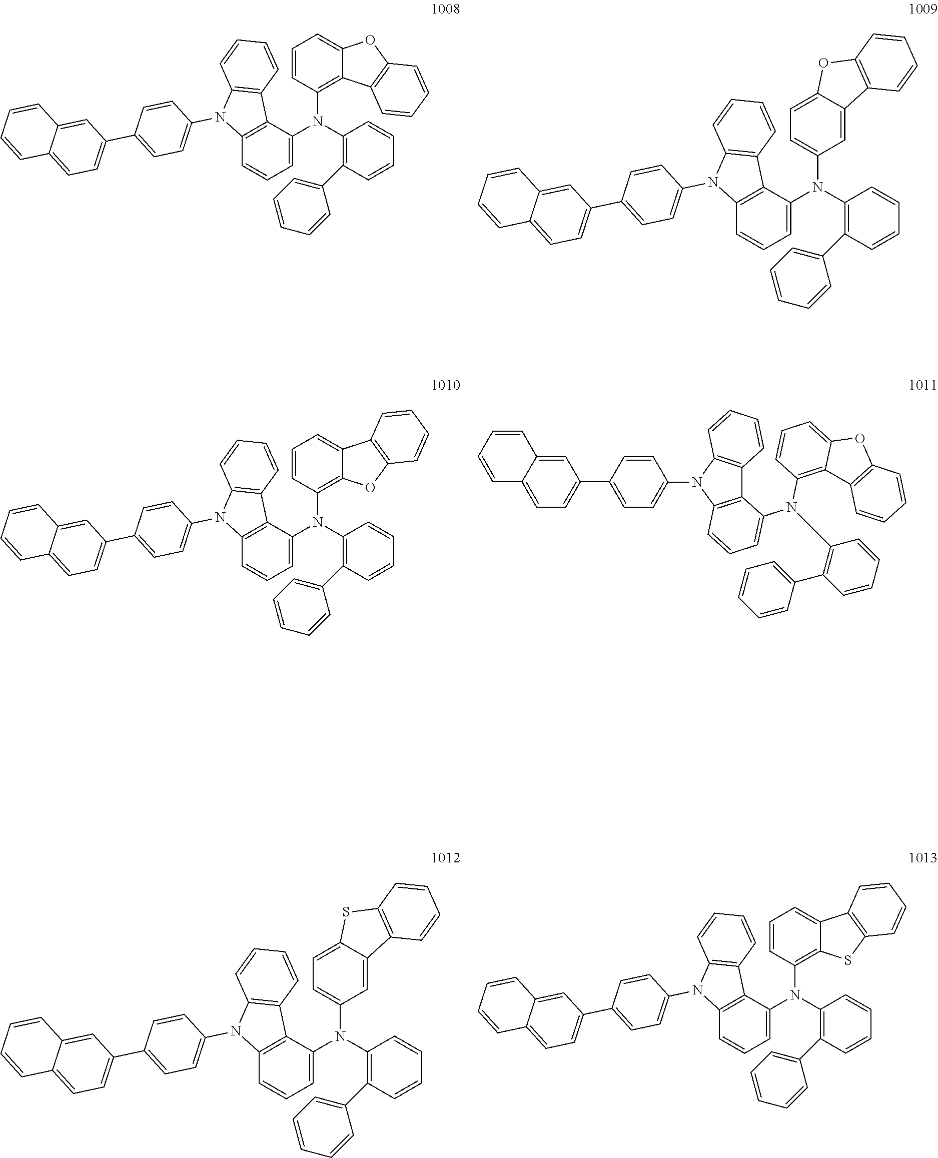

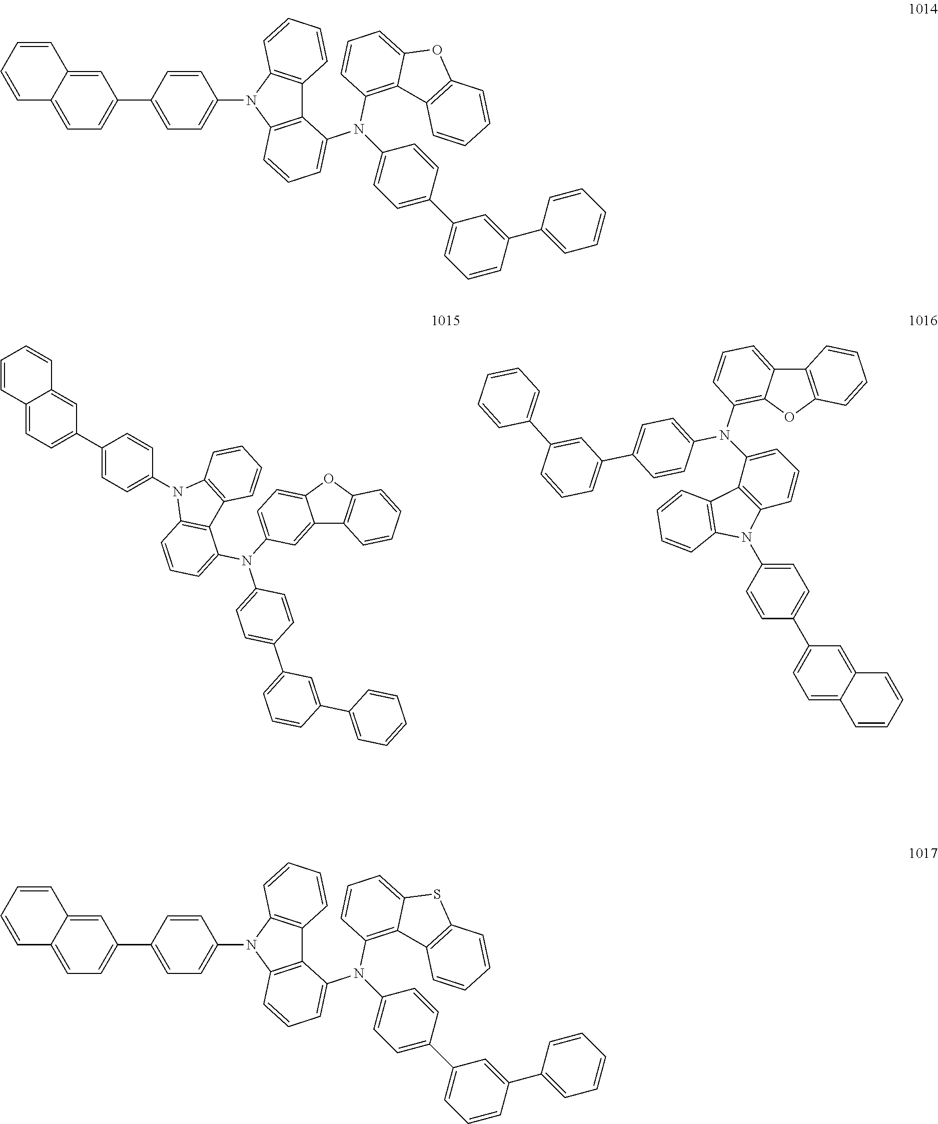 [SMILES: c1ccc(-c2cccc(-c3ccc(N(c4cccc5oc6ccccc6c45)c4cccc5c4c4ccccc4n5-c4ccc(-c5ccc6ccccc6c5)cc4)cc3)c2)cc1 ; c1ccc(-c2cccc(-c3ccc(N(c4ccc5oc6ccccc6c5c4)c4cccc5c4c4ccccc4n5-c4ccc(-c5ccc6ccccc6c5)cc4)cc3)c2)cc1 ; c1ccc(-c2cccc(-c3ccc(N(c4cccc5c4oc4ccccc45)c4cccc5c4c4ccccc4n5-c4ccc(-c5ccc6ccccc6c5)cc4)cc3)c2)cc1 ; c1ccc(-c2cccc(-c3ccc(N(c4cccc5sc6ccccc6c45)c4cccc5c4c4ccccc4n5-c4ccc(-c5ccc6ccccc6c5)cc4)cc3)c2)cc1]

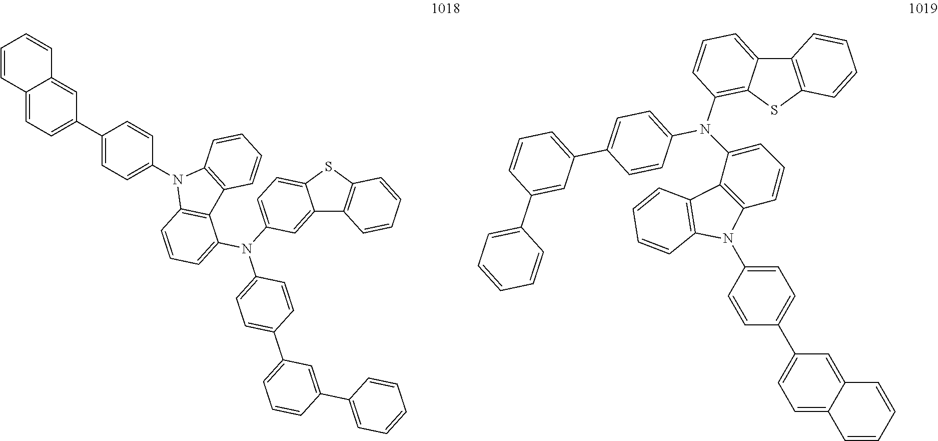 [SMILES: c1ccc(-c2cccc(-c3ccc(N(c4ccc5sc6ccccc6c5c4)c4cccc5c4c4ccccc4n5-c4ccc(-c5ccc6ccccc6c5)cc4)cc3)c2)cc1 ; c1ccc(-c2cccc(-c3ccc(N(c4cccc5c4sc4ccccc45)c4cccc5c4c4ccccc4n5-c4ccc(-c5ccc6ccccc6c5)cc4)cc3)c2)cc1]

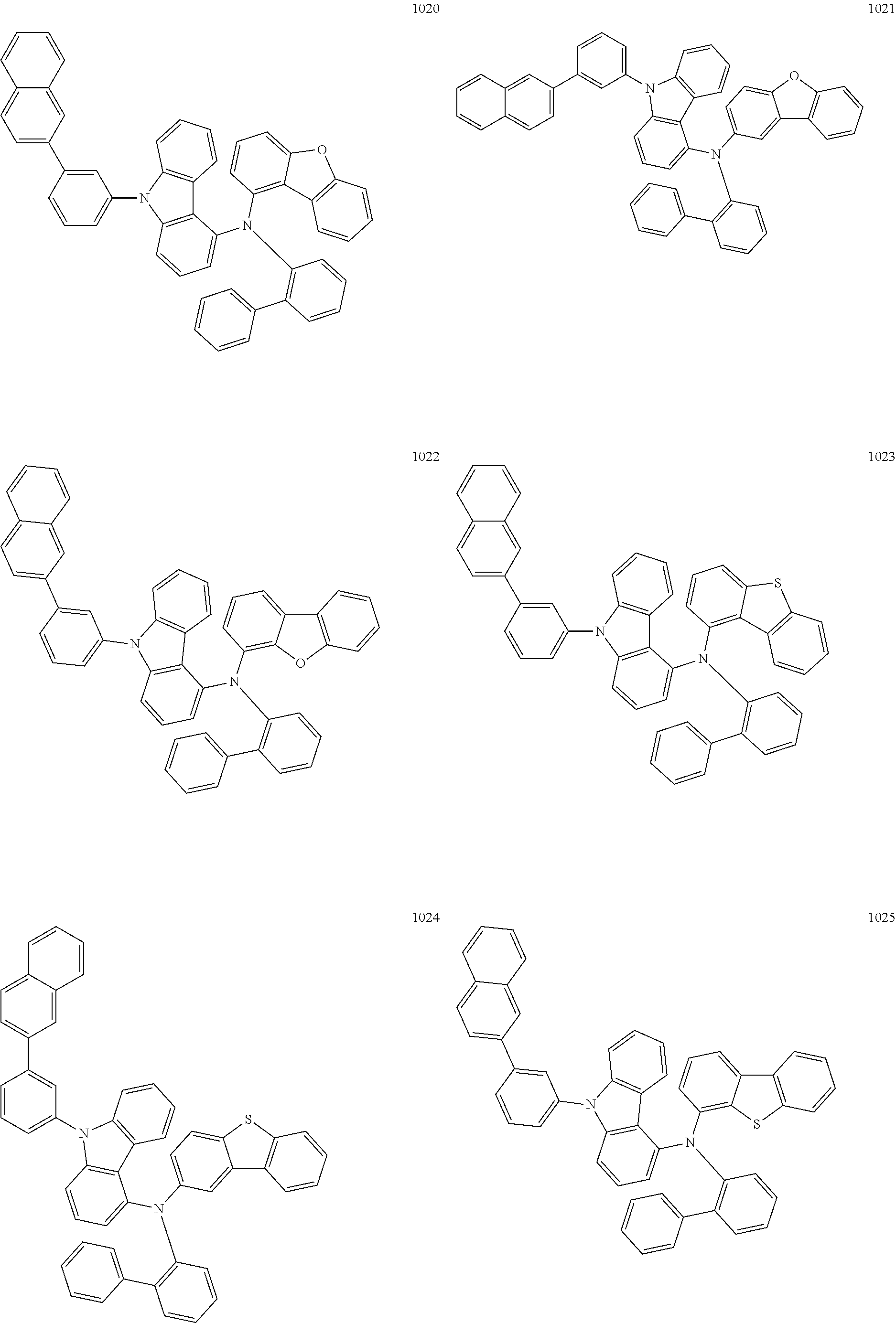

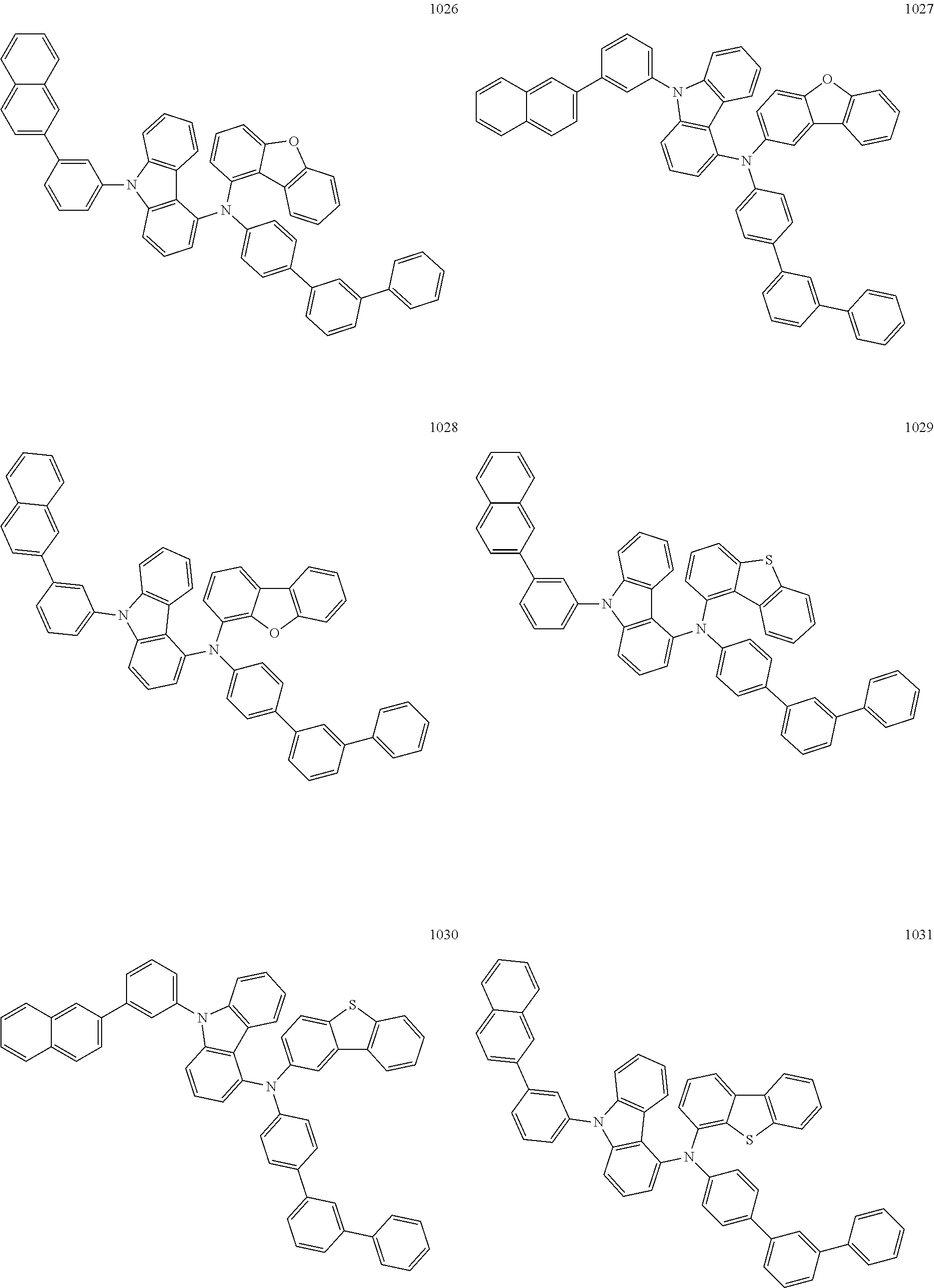

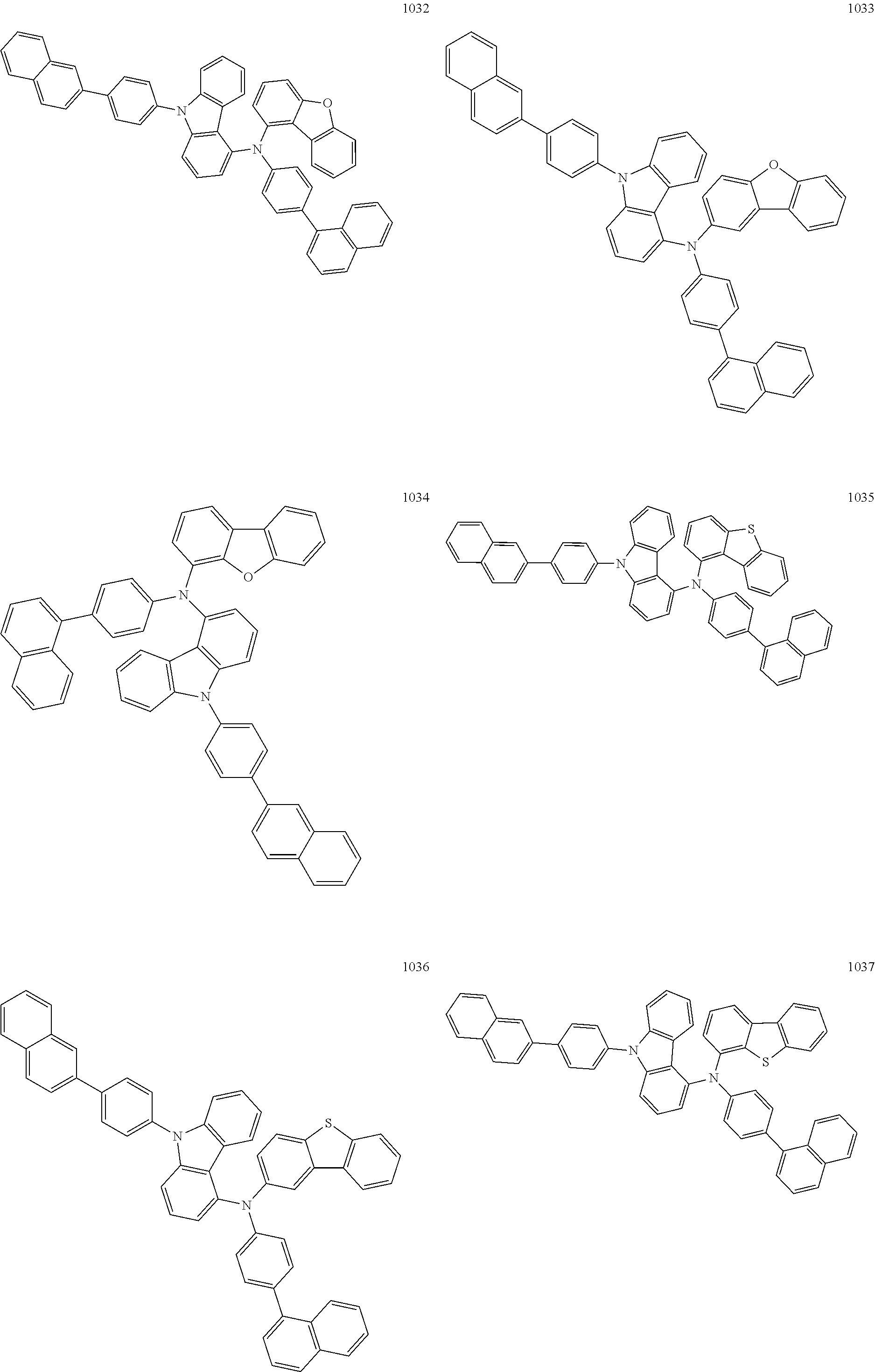

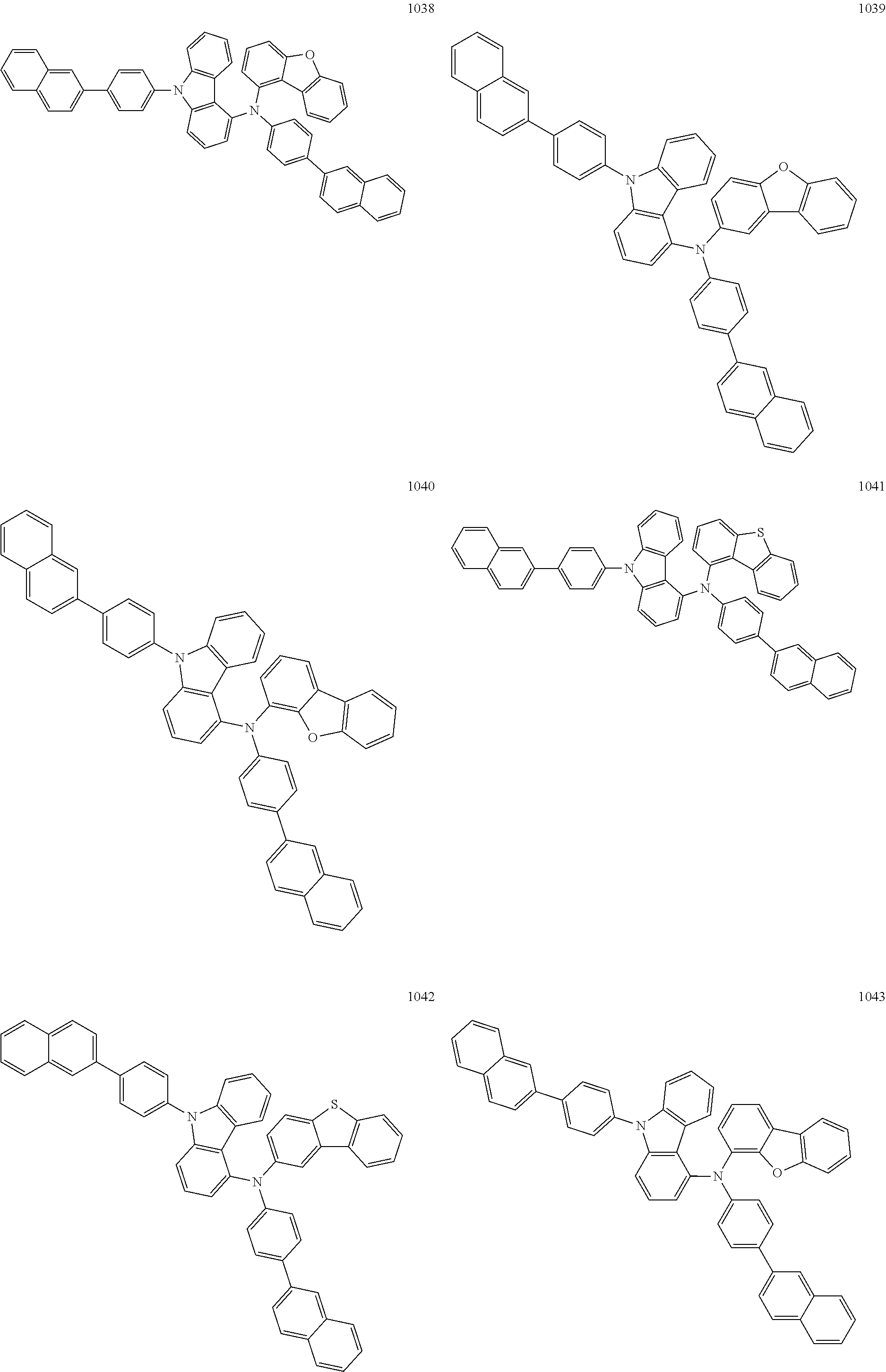

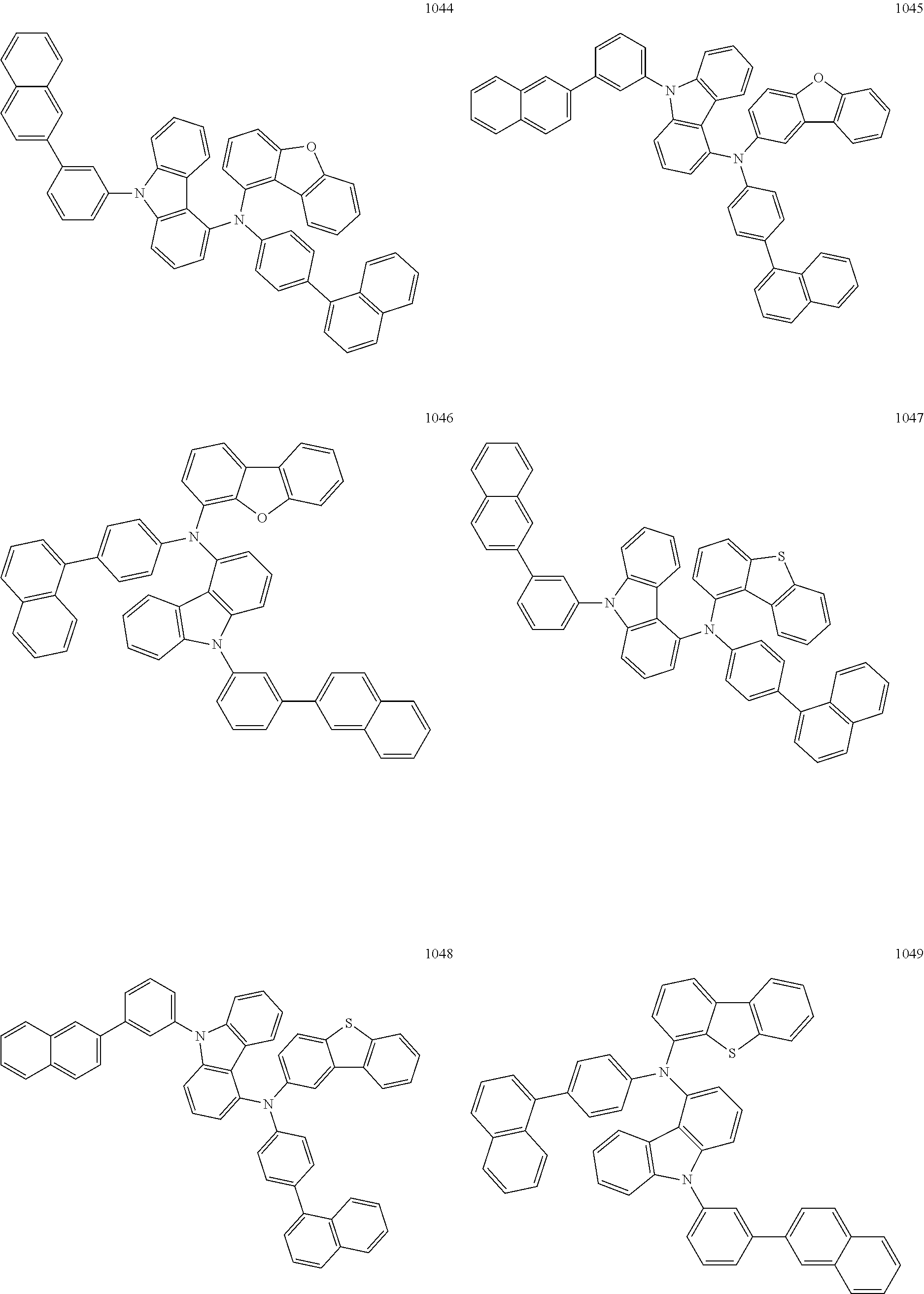

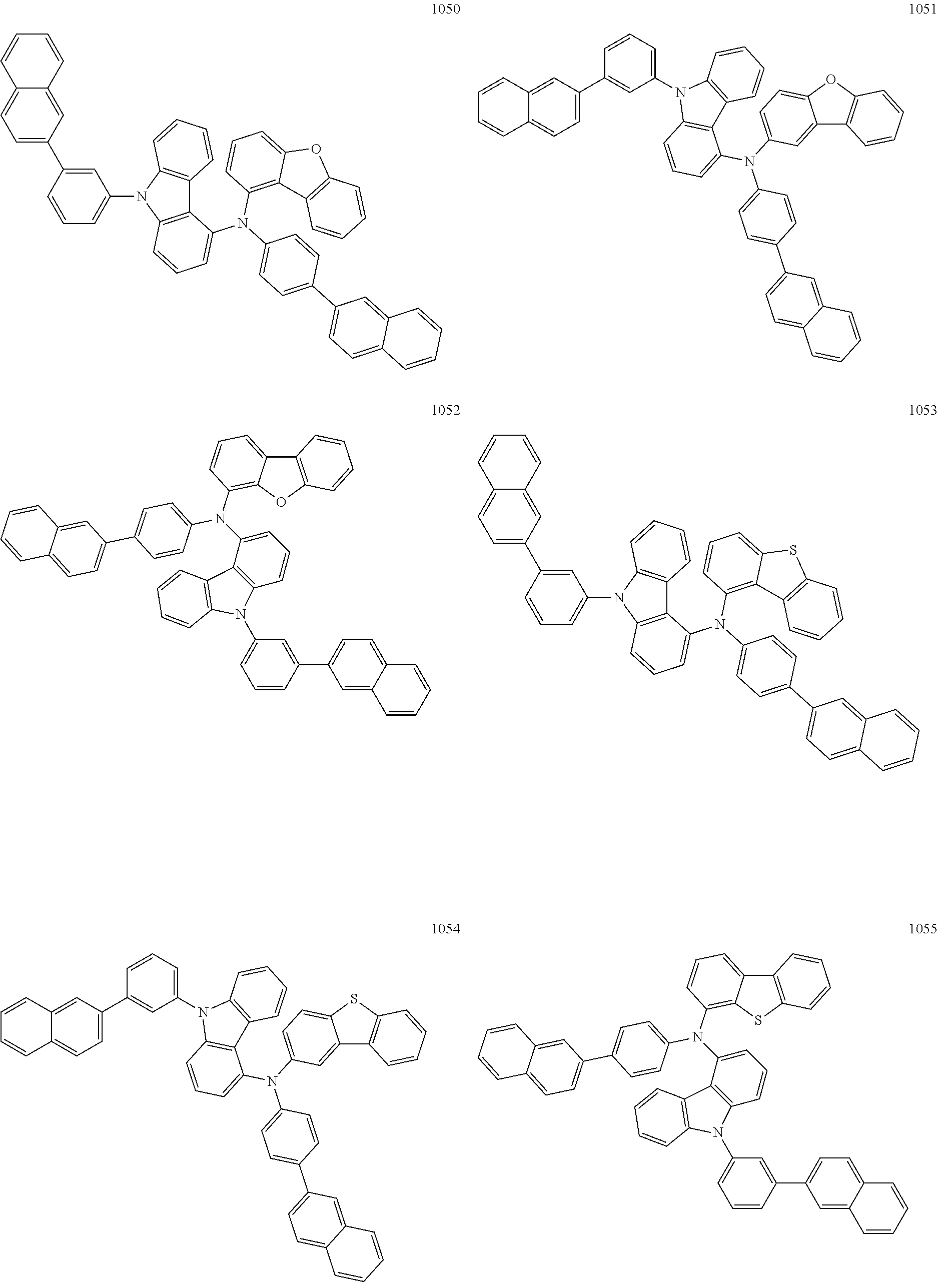

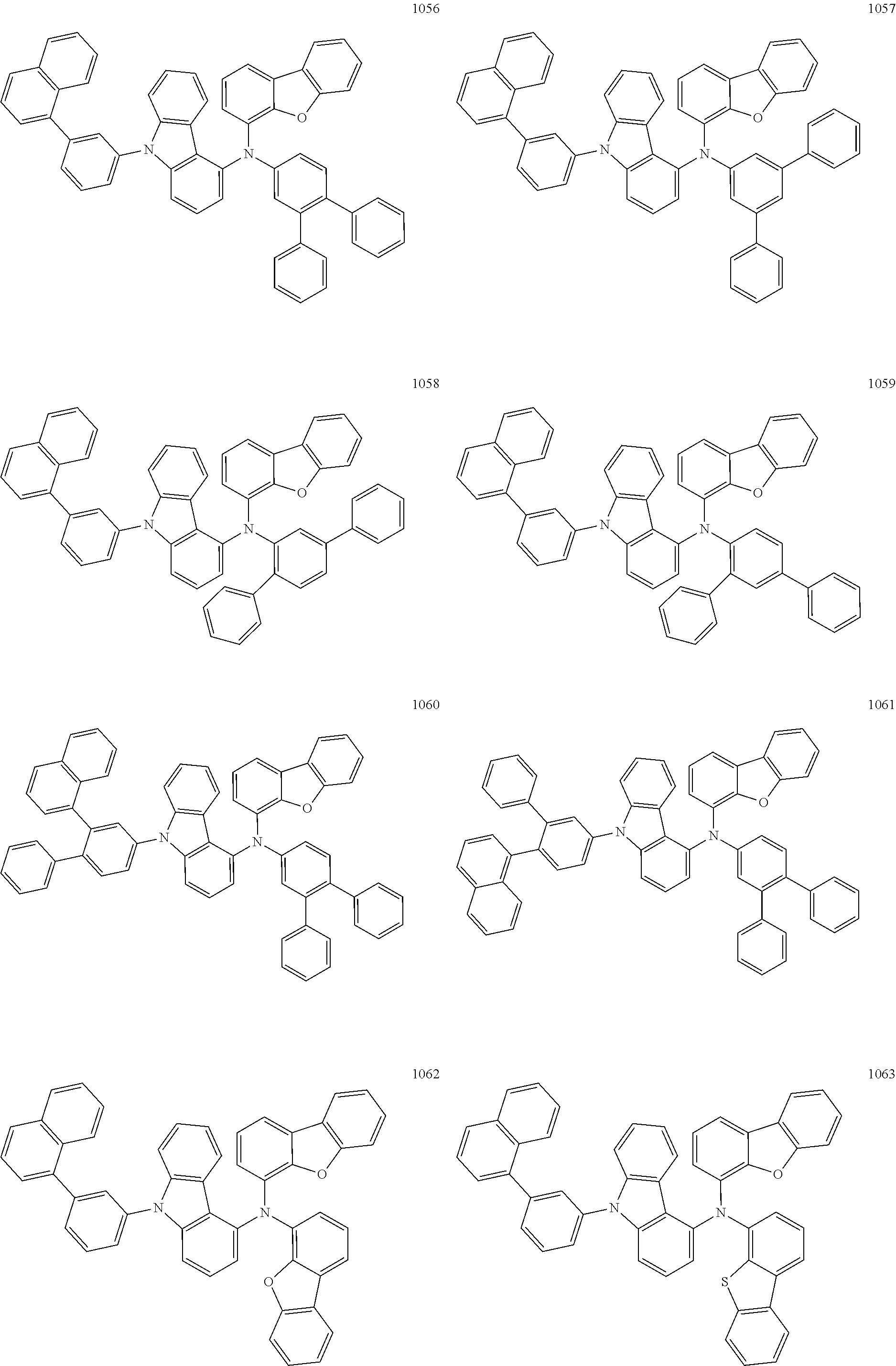

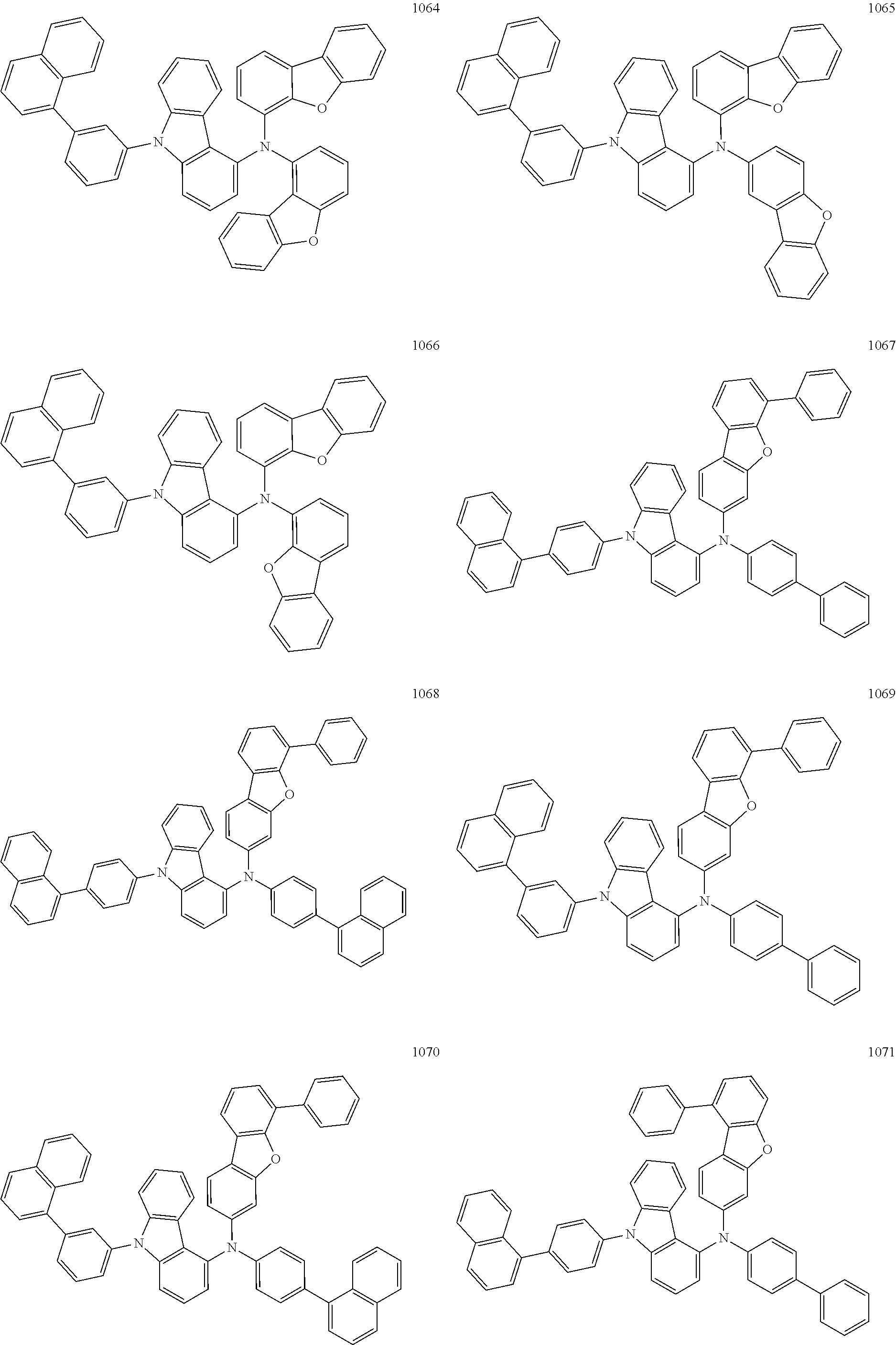

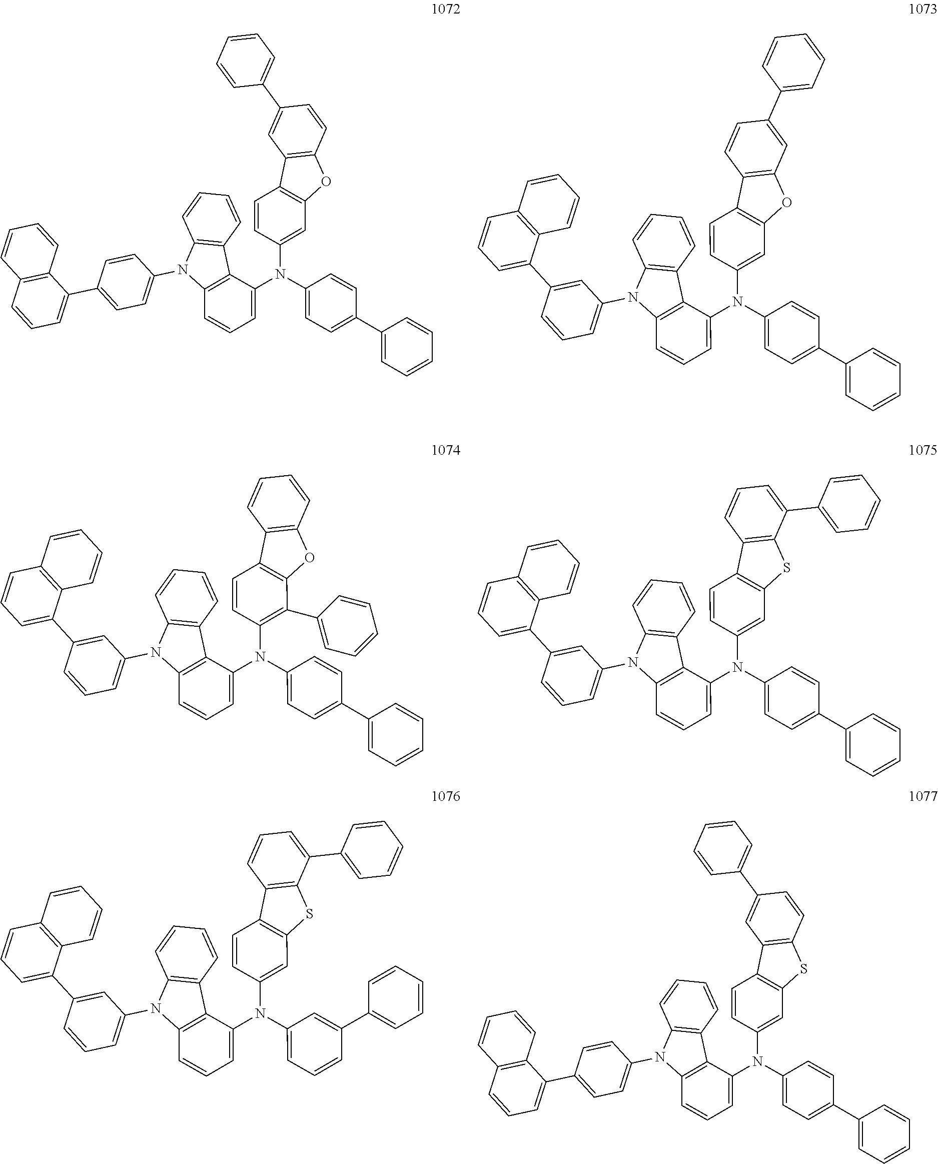

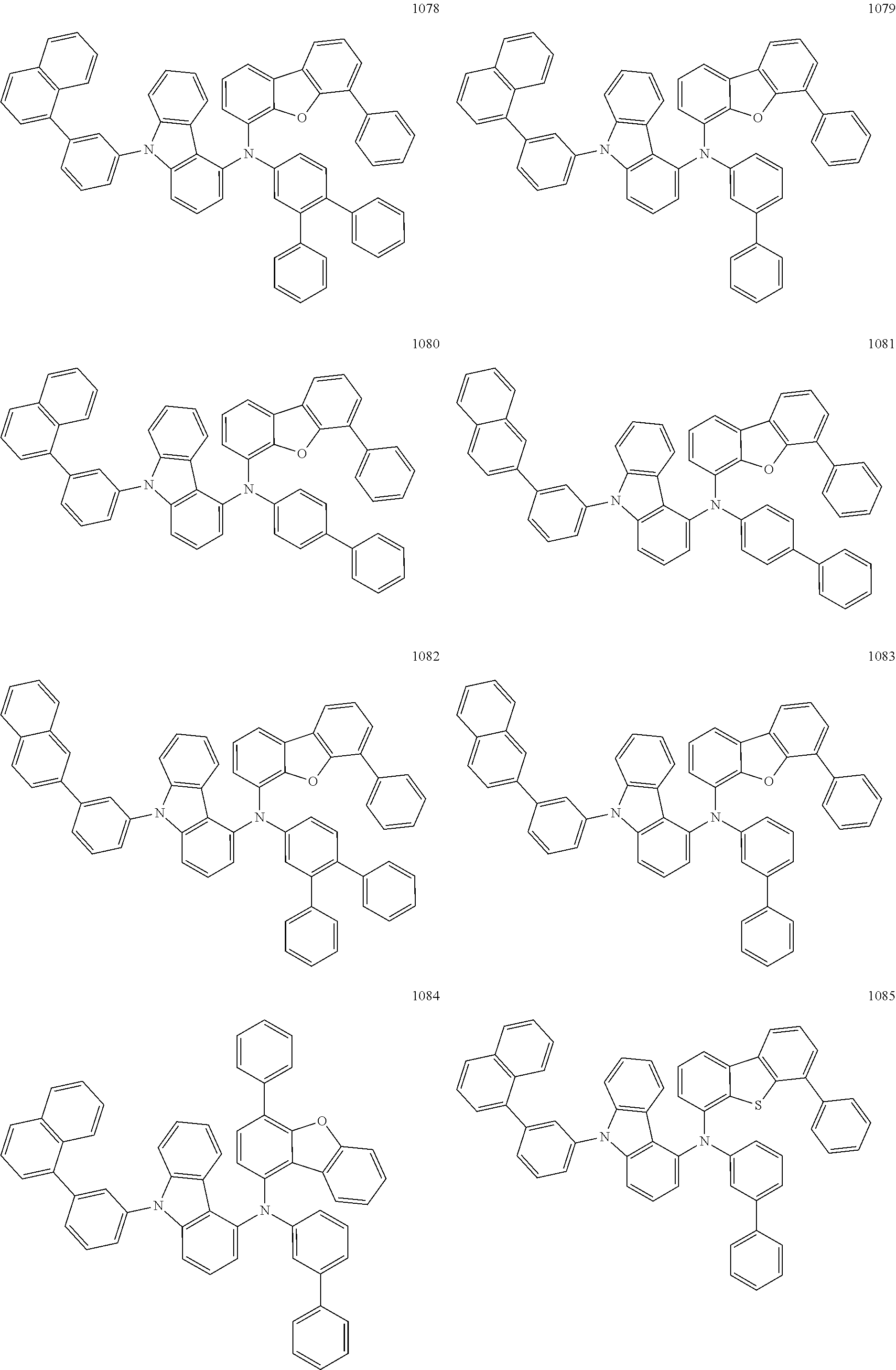

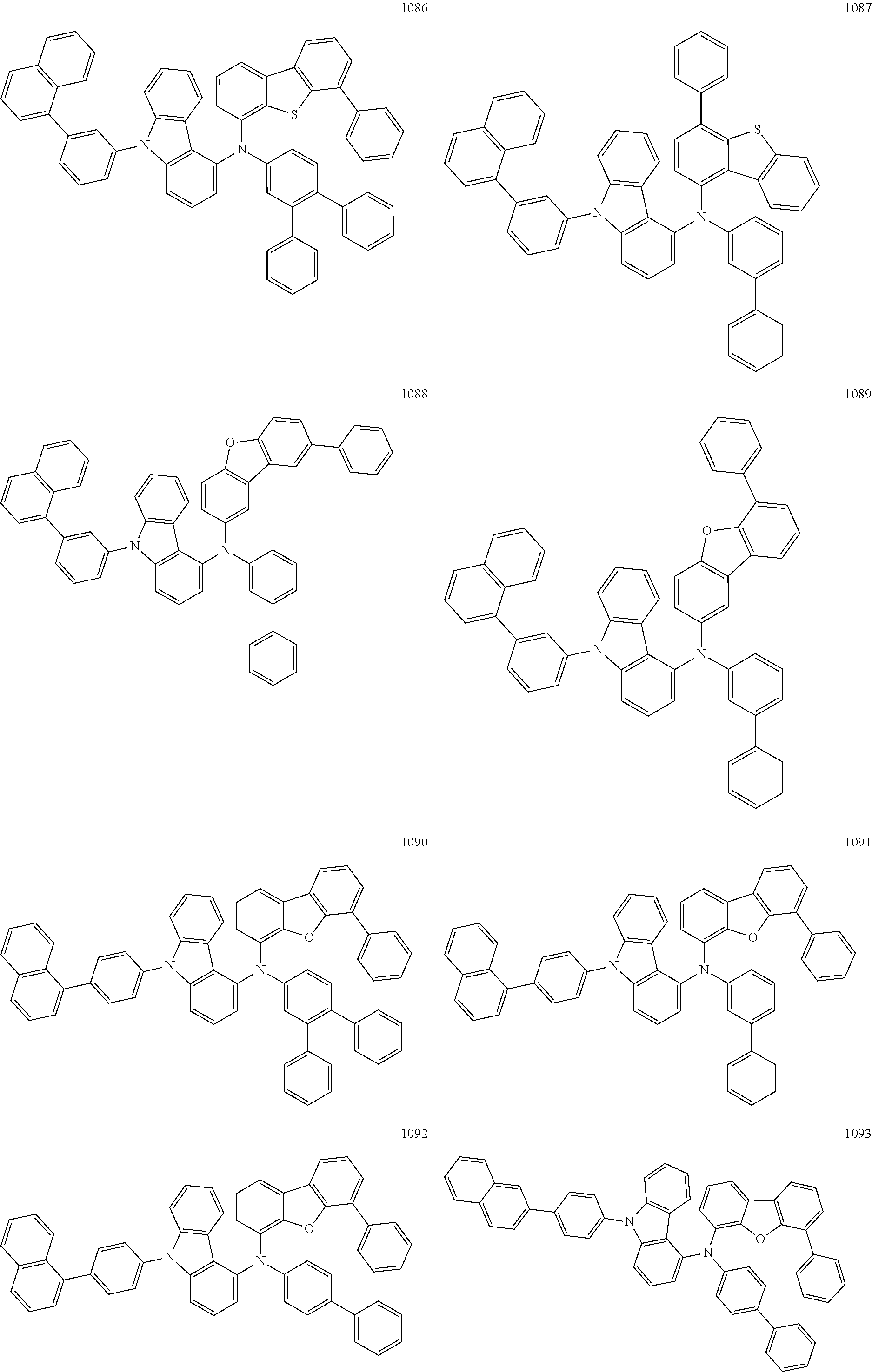

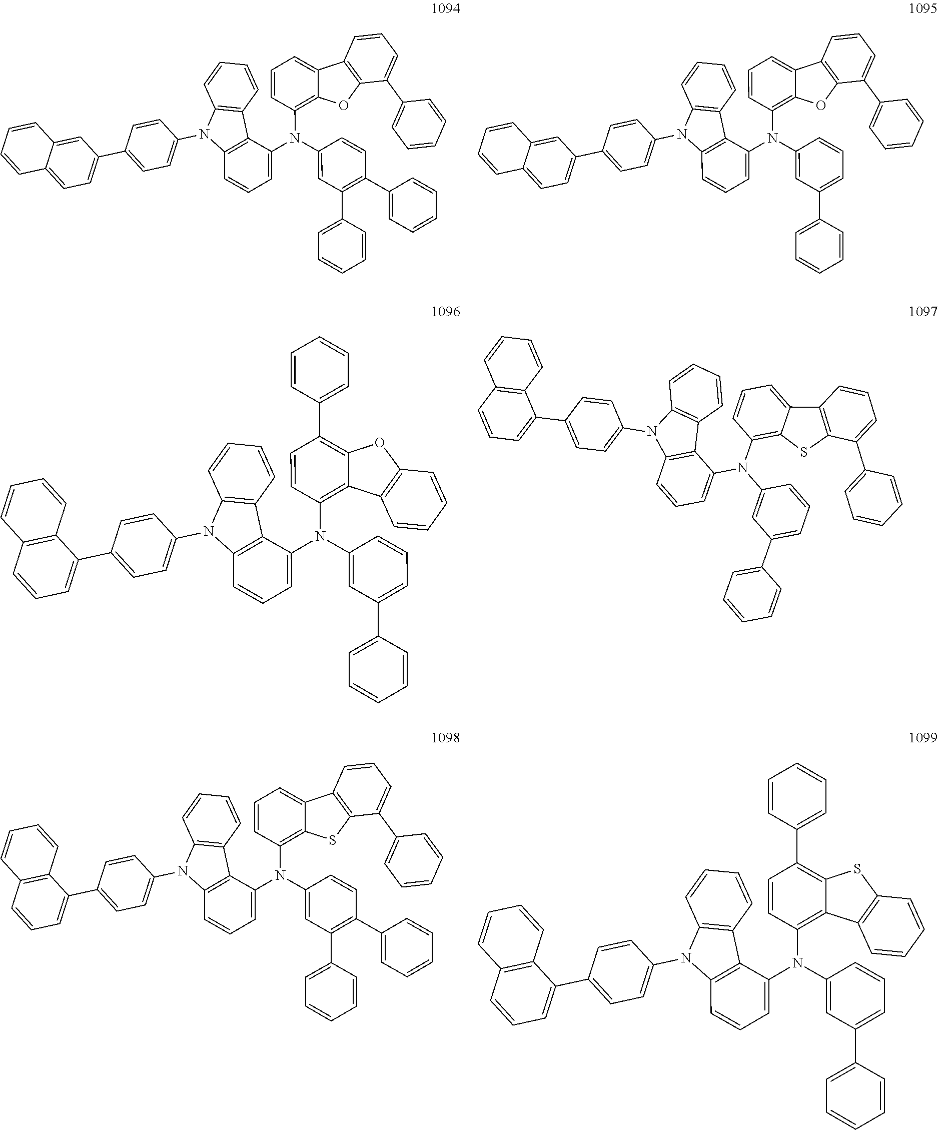

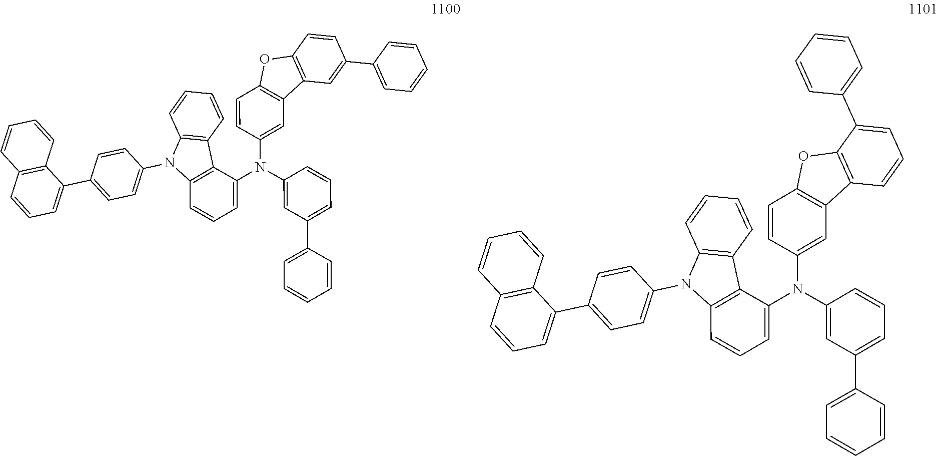 [SMILES: c1ccc(-c2cccc(N(c3ccc4oc5ccc(-c6ccccc6)cc5c4c3)c3cccc4c3c3ccccc3n4-c3ccc(-c4cccc5ccccc45)cc3)c2)cc1 ; c1ccc(-c2cccc(N(c3ccc4oc5c(-c6ccccc6)cccc5c4c3)c3cccc4c3c3ccccc3n4-c3ccc(-c4cccc5ccccc45)cc3)c2)cc1]

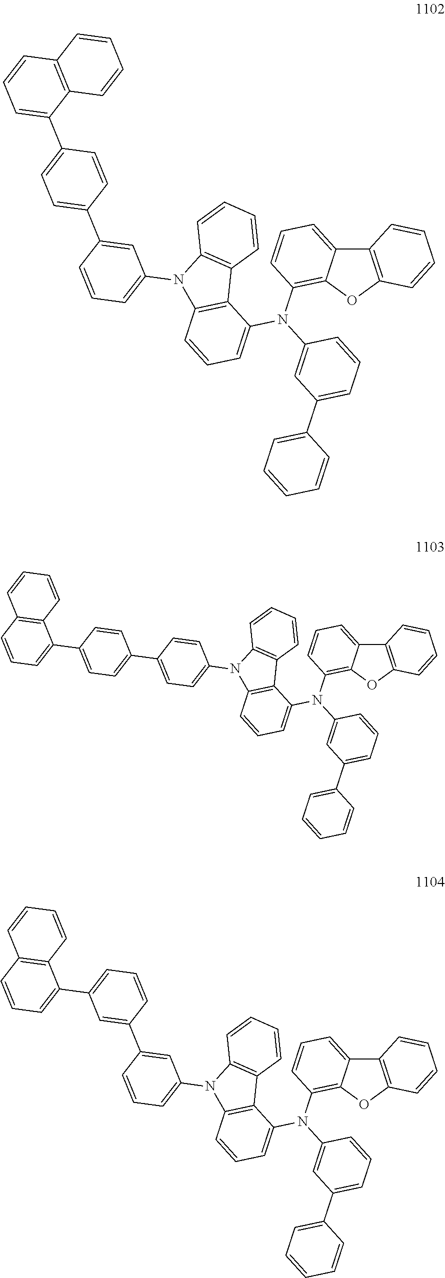 [SMILES: c1ccc(-c2cccc(N(c3cccc4c3oc3ccccc34)c3cccc4c3c3ccccc3n4-c3cccc(-c4ccc(-c5cccc6ccccc56)cc4)c3)c2)cc1 ; c1ccc(-c2cccc(N(c3cccc4c3oc3ccccc34)c3cccc4c3c3ccccc3n4-c3ccc(-c4ccc(-c5cccc6ccccc56)cc4)cc3)c2)cc1 ; c1ccc(-c2cccc(N(c3cccc4c3oc3ccccc34)c3cccc4c3c3ccccc3n4-c3cccc(-c4cccc(-c5cccc6ccccc56)c4)c3)c2)cc1]

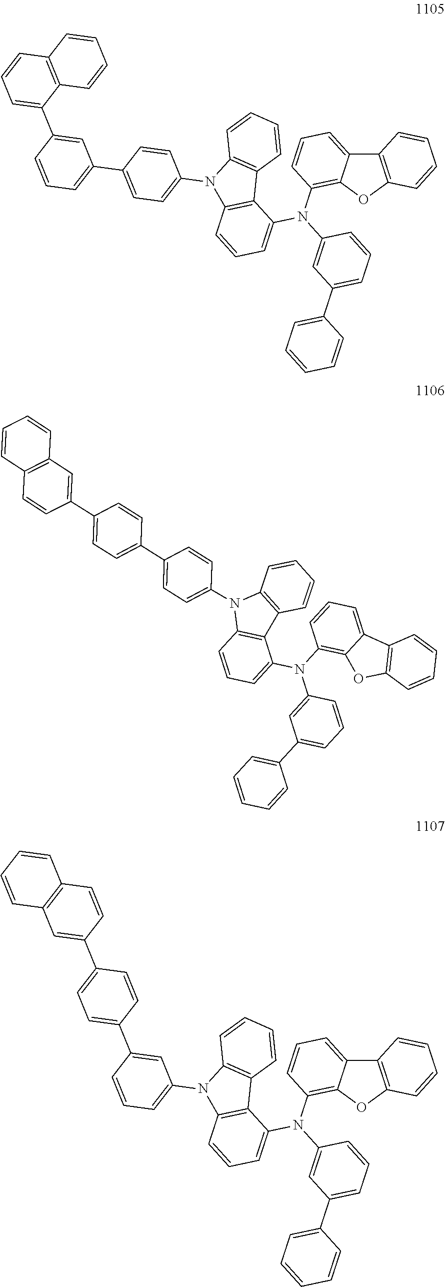 [SMILES: c1ccc(-c2cccc(N(c3cccc4c3oc3ccccc34)c3cccc4c3c3ccccc3n4-c3ccc(-c4cccc(-c5cccc6ccccc56)c4)cc3)c2)cc1 ; c1ccc(-c2cccc(N(c3cccc4c3oc3ccccc34)c3cccc4c3c3ccccc3n4-c3ccc(-c4ccc(-c5ccc6ccccc6c5)cc4)cc3)c2)cc1 ; c1ccc(-c2cccc(N(c3cccc4c3oc3ccccc34)c3cccc4c3c3ccccc3n4-c3cccc(-c4ccc(-c5ccc6ccccc6c5)cc4)c3)c2)cc1]

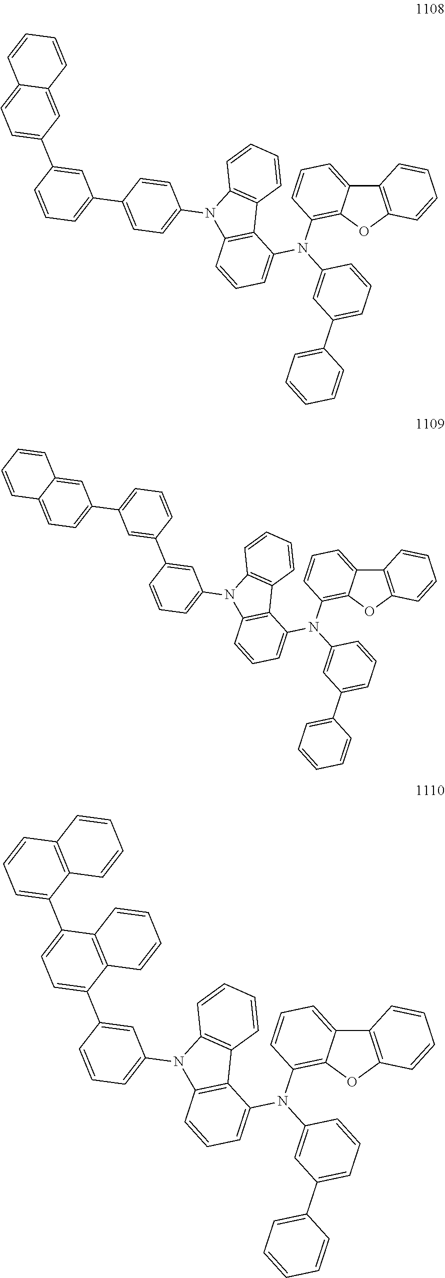 [SMILES: c1ccc(-c2cccc(N(c3cccc4c3oc3ccccc34)c3cccc4c3c3ccccc3n4-c3ccc(-c4cccc(-c5ccc6ccccc6c5)c4)cc3)c2)cc1 ; c1ccc(-c2cccc(N(c3cccc4c3oc3ccccc34)c3cccc4c3c3ccccc3n4-c3cccc(-c4cccc(-c5ccc6ccccc6c5)c4)c3)c2)cc1 ; c1ccc(-c2cccc(N(c3cccc4c3oc3ccccc34)c3cccc4c3c3ccccc3n4-c3cccc(-c4ccc(-c5cccc6ccccc56)c5ccccc45)c3)c2)cc1]

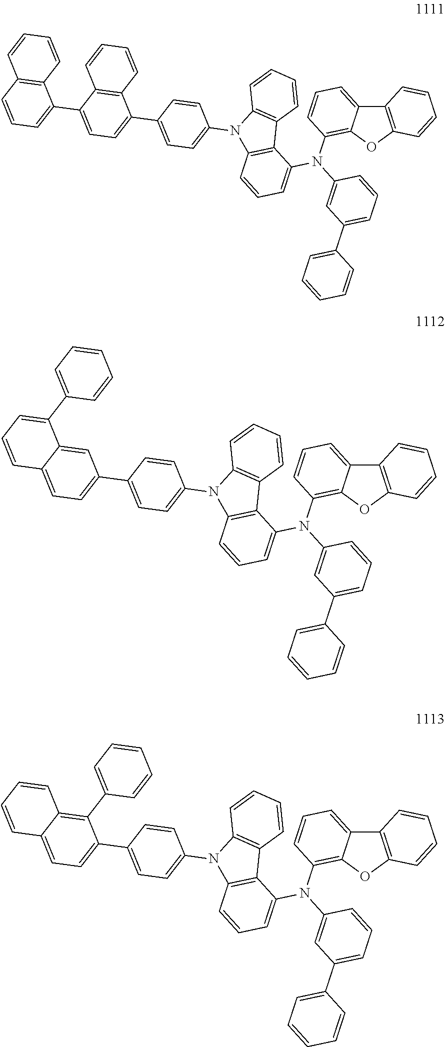 [SMILES: c1ccc(-c2cccc(N(c3cccc4c3oc3ccccc34)c3cccc4c3c3ccccc3n4-c3ccc(-c4ccc(-c5cccc6ccccc56)c5ccccc45)cc3)c2)cc1 ; c1ccc(-c2cccc(N(c3cccc4c3oc3ccccc34)c3cccc4c3c3ccccc3n4-c3ccc(-c4ccc5cccc(-c6ccccc6)c5c4)cc3)c2)cc1 ; c1ccc(-c2cccc(N(c3cccc4c3oc3ccccc34)c3cccc4c3c3ccccc3n4-c3ccc(-c4ccc5ccccc5c4-c4ccccc4)cc3)c2)cc1]

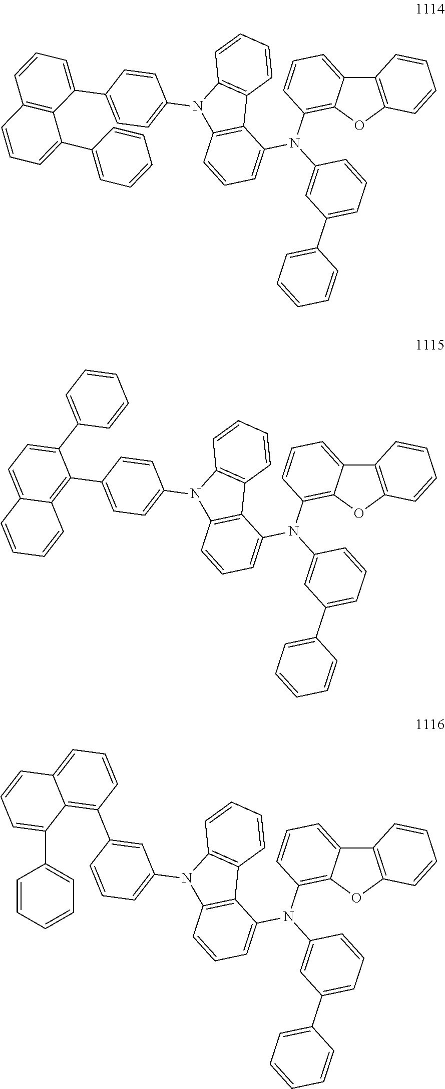 [SMILES: c1ccc(-c2cccc(N(c3cccc4c3oc3ccccc34)c3cccc4c3c3ccccc3n4-c3ccc(-c4cccc5cccc(-c6ccccc6)c45)cc3)c2)cc1 ; c1ccc(-c2cccc(N(c3cccc4c3oc3ccccc34)c3cccc4c3c3ccccc3n4-c3ccc(-c4c(-c5ccccc5)ccc5ccccc45)cc3)c2)cc1 ; c1ccc(-c2cccc(N(c3cccc4c3oc3ccccc34)c3cccc4c3c3ccccc3n4-c3cccc(-c4cccc5cccc(-c6ccccc6)c45)c3)c2)cc1]

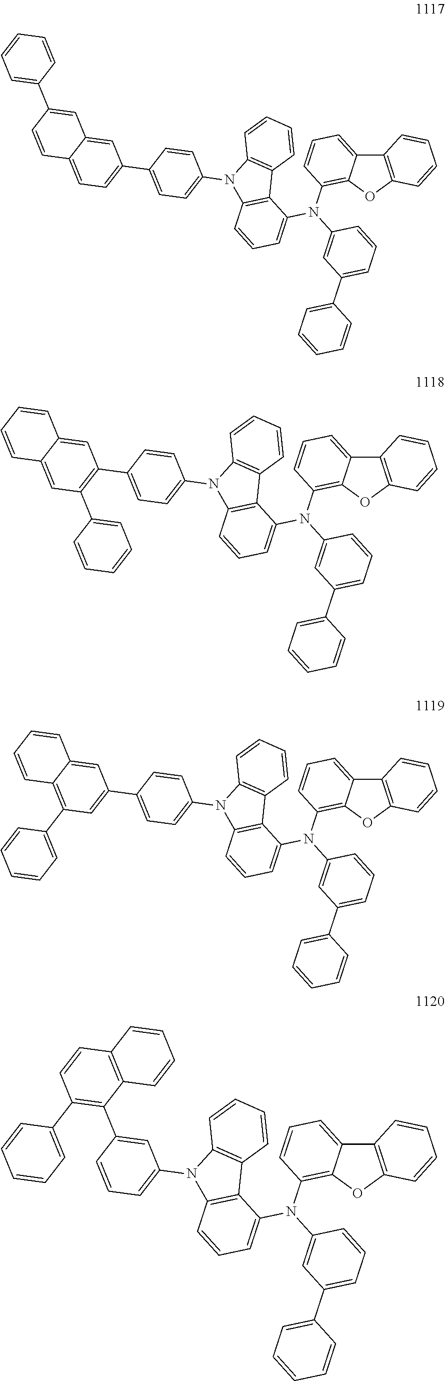 [SMILES: c1ccc(-c2cccc(N(c3cccc4c3oc3ccccc34)c3cccc4c3c3ccccc3n4-c3ccc(-c4ccc5ccc(-c6ccccc6)cc5c4)cc3)c2)cc1 ; c1ccc(-c2cccc(N(c3cccc4c3oc3ccccc34)c3cccc4c3c3ccccc3n4-c3ccc(-c4cc5ccccc5cc4-c4ccccc4)cc3)c2)cc1 ; c1ccc(-c2cccc(N(c3cccc4c3oc3ccccc34)c3cccc4c3c3ccccc3n4-c3ccc(-c4cc(-c5ccccc5)c5ccccc5c4)cc3)c2)cc1 ; c1ccc(-c2cccc(N(c3cccc4c3oc3ccccc34)c3cccc4c3c3ccccc3n4-c3cccc(-c4c(-c5ccccc5)ccc5ccccc45)c3)c2)cc1]

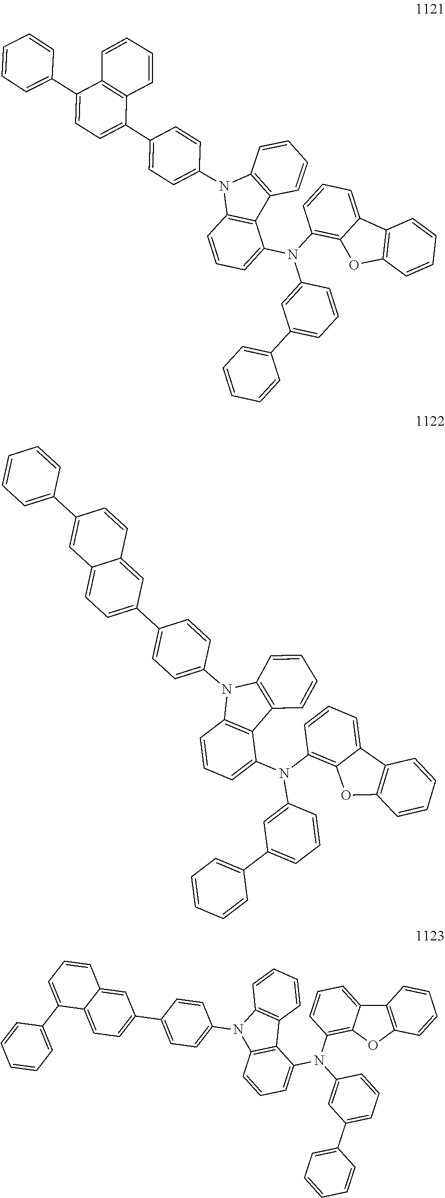 [SMILES: c1ccc(-c2cccc(N(c3cccc4c3oc3ccccc34)c3cccc4c3c3ccccc3n4-c3ccc(-c4ccc(-c5ccccc5)c5ccccc45)cc3)c2)cc1 ; c1ccc(-c2cccc(N(c3cccc4c3oc3ccccc34)c3cccc4c3c3ccccc3n4-c3ccc(-c4ccc5cc(-c6ccccc6)ccc5c4)cc3)c2)cc1 ; c1ccc(-c2cccc(N(c3cccc4c3oc3ccccc34)c3cccc4c3c3ccccc3n4-c3ccc(-c4ccc5c(-c6ccccc6)cccc5c4)cc3)c2)cc1]

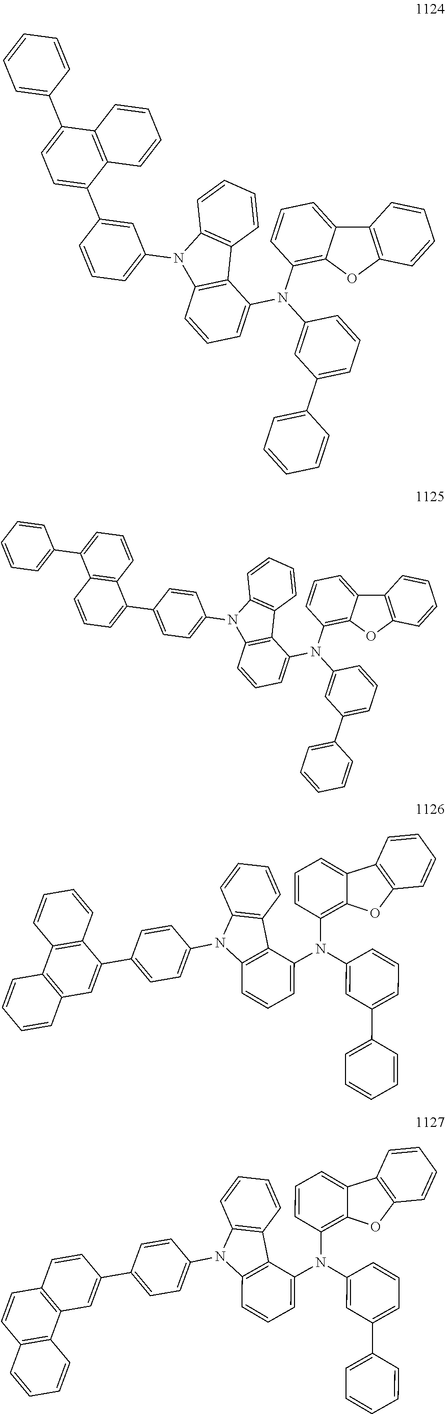 [SMILES: c1ccc(-c2cccc(N(c3cccc4c3oc3ccccc34)c3cccc4c3c3ccccc3n4-c3cccc(-c4ccc(-c5ccccc5)c5ccccc45)c3)c2)cc1 ; c1ccc(-c2cccc(N(c3cccc4c3oc3ccccc34)c3cccc4c3c3ccccc3n4-c3ccc(-c4cccc5c(-c6ccccc6)cccc45)cc3)c2)cc1 ; c1ccc(-c2cccc(N(c3cccc4c3oc3ccccc34)c3cccc4c3c3ccccc3n4-c3ccc(-c4cc5ccccc5c5ccccc45)cc3)c2)cc1 ; c1ccc(-c2cccc(N(c3cccc4c3oc3ccccc34)c3cccc4c3c3ccccc3n4-c3ccc(-c4ccc5ccc6ccccc6c5c4)cc3)c2)cc1]

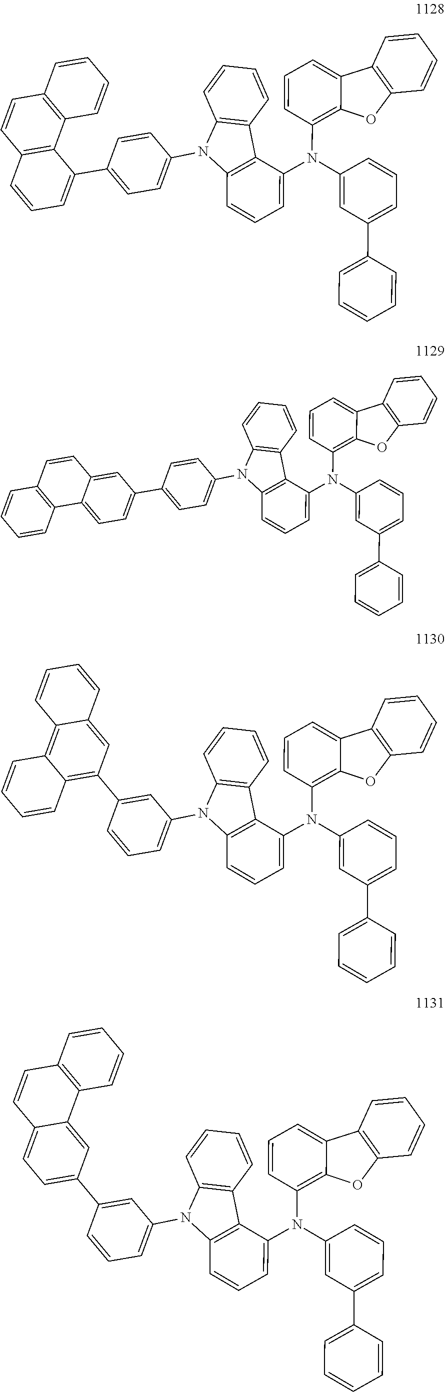 [SMILES: c1ccc(-c2cccc(N(c3cccc4c3oc3ccccc34)c3cccc4c3c3ccccc3n4-c3ccc(-c4cccc5ccc6ccccc6c45)cc3)c2)cc1 ; c1ccc(-c2cccc(N(c3cccc4c3oc3ccccc34)c3cccc4c3c3ccccc3n4-c3ccc(-c4ccc5c(ccc6ccccc65)c4)cc3)c2)cc1 ; c1ccc(-c2cccc(N(c3cccc4c3oc3ccccc34)c3cccc4c3c3ccccc3n4-c3cccc(-c4cc5ccccc5c5ccccc45)c3)c2)cc1 ; c1ccc(-c2cccc(N(c3cccc4c3oc3ccccc34)c3cccc4c3c3ccccc3n4-c3cccc(-c4ccc5ccc6ccccc6c5c4)c3)c2)cc1]

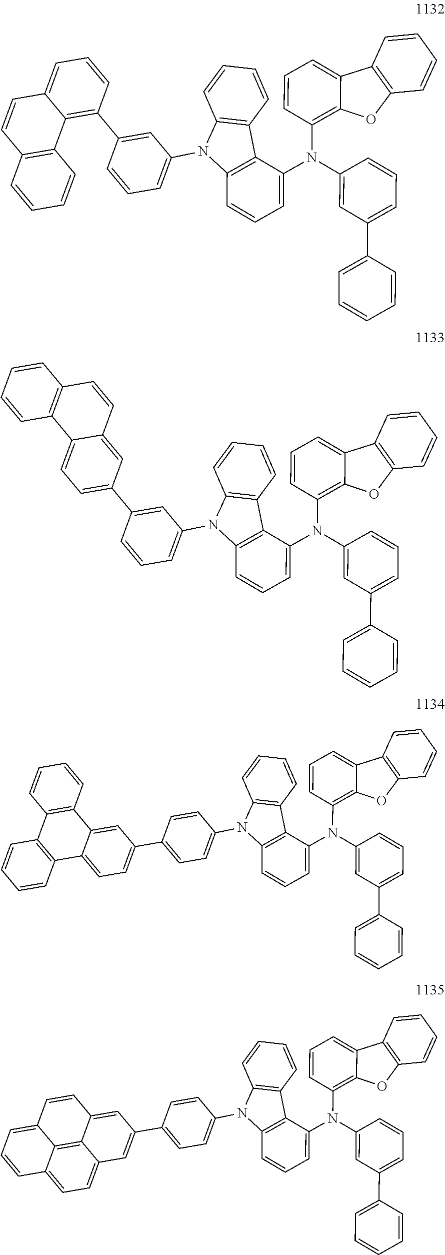 [SMILES: c1ccc(-c2cccc(N(c3cccc4c3oc3ccccc34)c3cccc4c3c3ccccc3n4-c3cccc(-c4cccc5ccc6ccccc6c45)c3)c2)cc1 ; c1ccc(-c2cccc(N(c3cccc4c3oc3ccccc34)c3cccc4c3c3ccccc3n4-c3cccc(-c4ccc5c(ccc6ccccc65)c4)c3)c2)cc1 ; c1ccc(-c2cccc(N(c3cccc4c3oc3ccccc34)c3cccc4c3c3ccccc3n4-c3ccc(-c4ccc5c6ccccc6c6ccccc6c5c4)cc3)c2)cc1 ; c1ccc(-c2cccc(N(c3cccc4c3oc3ccccc34)c3cccc4c3c3ccccc3n4-c3ccc(-c4cc5ccc6cccc7ccc(c4)c5c67)cc3)c2)cc1]

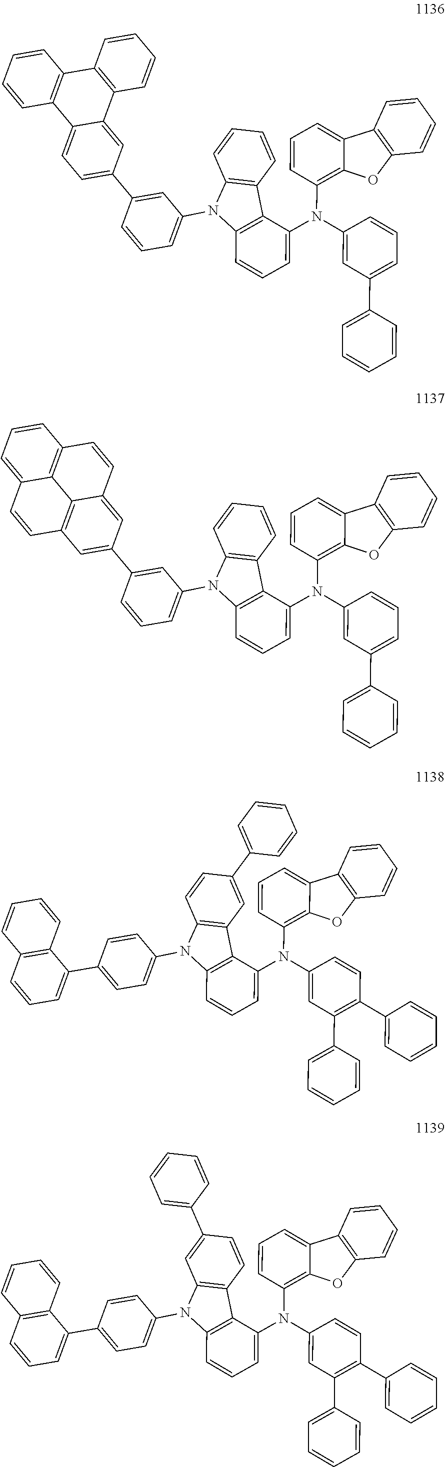 [SMILES: c1ccc(-c2cccc(N(c3cccc4c3oc3ccccc34)c3cccc4c3c3ccccc3n4-c3cccc(-c4ccc5c6ccccc6c6ccccc6c5c4)c3)c2)cc1 ; c1ccc(-c2cccc(N(c3cccc4c3oc3ccccc34)c3cccc4c3c3ccccc3n4-c3cccc(-c4cc5ccc6cccc7ccc(c4)c5c67)c3)c2)cc1 ; c1ccc(-c2ccc3c(c2)c2c(N(c4ccc(-c5ccccc5)c(-c5ccccc5)c4)c4cccc5c4oc4ccccc45)cccc2n3-c2ccc(-c3cccc4ccccc34)cc2)cc1 ; c1ccc(-c2ccc3c4c(N(c5ccc(-c6ccccc6)c(-c6ccccc6)c5)c5cccc6c5oc5ccccc56)cccc4n(-c4ccc(-c5cccc6ccccc56)cc4)c3c2)cc1]

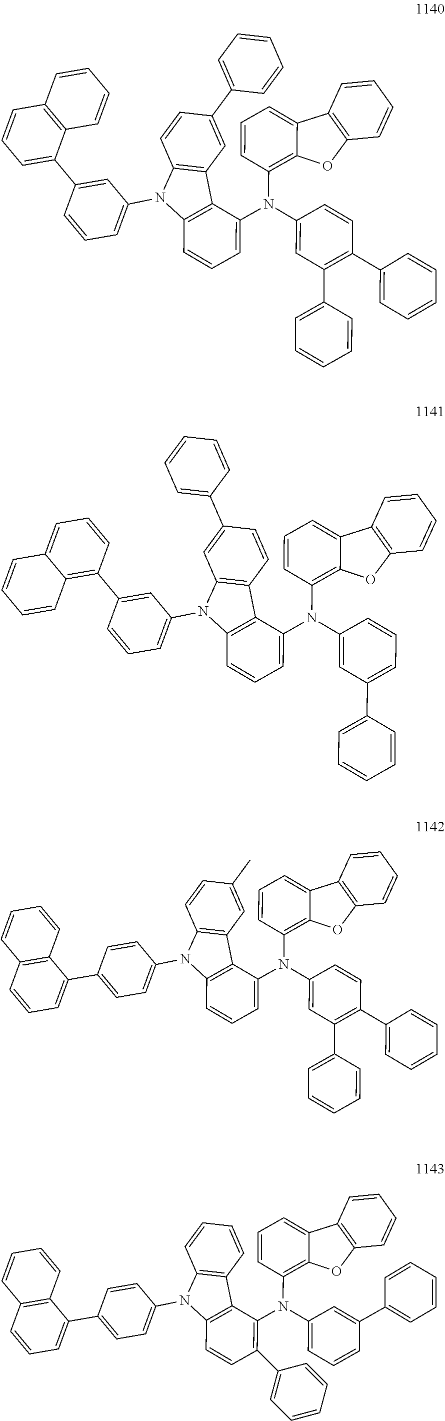 [SMILES: c1ccc(-c2ccc3c(c2)c2c(N(c4ccc(-c5ccccc5)c(-c5ccccc5)c4)c4cccc5c4oc4ccccc45)cccc2n3-c2cccc(-c3cccc4ccccc34)c2)cc1 ; c1ccc(-c2cccc(N(c3cccc4c3oc3ccccc34)c3cccc4c3c3ccc(-c5ccccc5)cc3n4-c3cccc(-c4cccc5ccccc45)c3)c2)cc1 ; Cc1ccc2c(c1)c1c(N(c3ccc(-c4ccccc4)c(-c4ccccc4)c3)c3cccc4c3oc3ccccc34)cccc1n2-c1ccc(-c2cccc3ccccc23)cc1 ; c1ccc(-c2cccc(N(c3cccc4c3oc3ccccc34)c3c(-c4ccccc4)ccc4c3c3ccccc3n4-c3ccc(-c4cccc5ccccc45)cc3)c2)cc1]

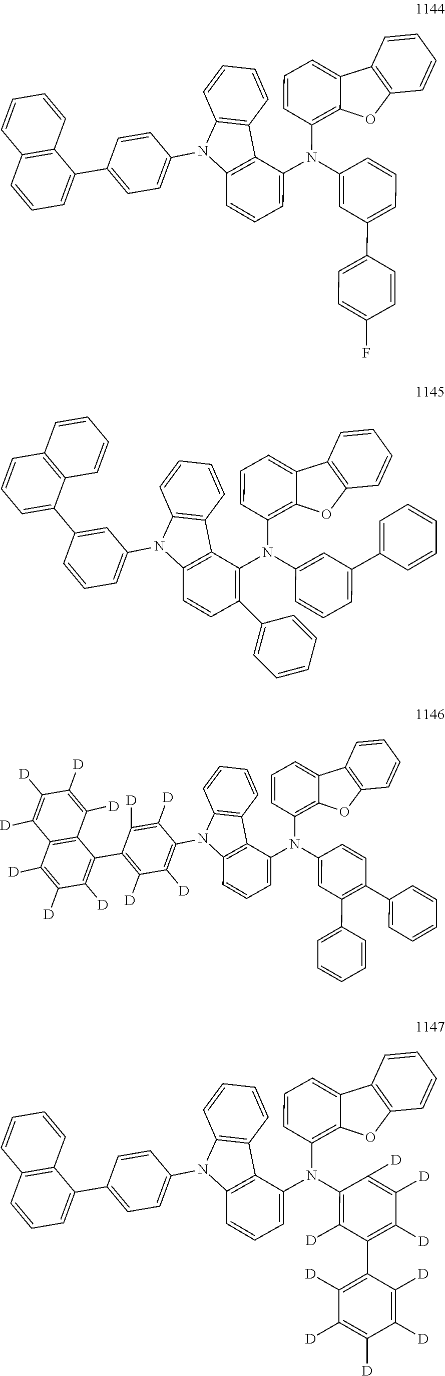 [SMILES: Fc1ccc(-c2cccc(N(c3cccc4c3oc3ccccc34)c3cccc4c3c3ccccc3n4-c3ccc(-c4cccc5ccccc45)cc3)c2)cc1 ; c1ccc(-c2cccc(N(c3cccc4c3oc3ccccc34)c3c(-c4ccccc4)ccc4c3c3ccccc3n4-c3cccc(-c4cccc5ccccc45)c3)c2)cc1 ; [2H]c1c([2H])c(-n2c3ccccc3c3c(N(c4ccc(-c5ccccc5)c(-c5ccccc5)c4)c4cccc5c4oc4ccccc45)cccc32)c([2H])c([2H])c1-c1c([2H])c([2H])c([2H])c2c([2H])c([2H])c([2H])c([2H])c12 ; [2H]c1c([2H])c([2H])c(-c2c([2H])c([2H])c([2H])c(N(c3cccc4c3oc3ccccc34)c3cccc4c3c3ccccc3n4-c3ccc(-c4cccc5ccccc45)cc3)c2[2H])c([2H])c1[2H]]

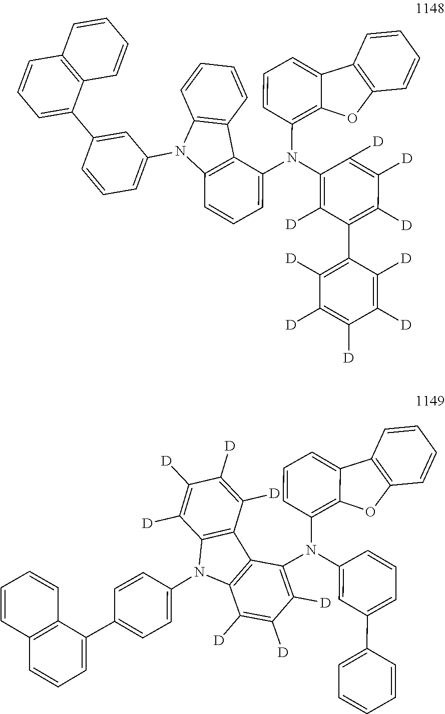 [SMILES: [2H]c1c([2H])c([2H])c(-c2c([2H])c([2H])c([2H])c(N(c3cccc4c3oc3ccccc34)c3cccc4c3c3ccccc3n4-c3cccc(-c4cccc5ccccc45)c3)c2[2H])c([2H])c1[2H] ; [2H]c1c([2H])c([2H])c2c(c1[2H])c1c(N(c3cccc(-c4ccccc4)c3)c3cccc4c3oc3ccccc34)c([2H])c([2H])c([2H])c1n2-c1ccc(-c2cccc3ccccc23)cc1]

In Compound Group 1, “D” is a deuterium atom.

The light emitting element ED of one or more embodiments may include at least one selected from among the amine compounds illustrated in Compound Group 1 in a hole transport region HTR. The amine compound according to one or more embodiments may include a first substituent, a second substituent, and a third substituent, connected with the nitrogen atom of the amine compound, and may accomplish the high efficiency and long lifetime of the light emitting element ED.

For example, the amine compound of one or more embodiments may include a first substituent including a carbazole moiety directly connected with the nitrogen atom of the amine, and a second substituent which is directly connected with the nitrogen atom of the amine and including a dibenzofuran moiety or a dibenzothiophene moiety. In some embodiments, an aryl group or a heteroaryl group may be included as a third substituent. For example, in the amine compound of one or more embodiments, a substituted or unsubstituted aryl group of 10 to 30 ring-forming carbon atoms (for example, a naphthyl group) may be connected at position 9 of the carbazole moiety of the first substituent via a phenylene linker, and may show excellent or suitable hole transport properties and improved electron tolerance, thereby showing properties of not being easily decomposed by heat. Accordingly, if the amine compound of one or more embodiments is utilized or applied in a light emitting element ED, element lifetime and emission efficiency may be improved.

The hole transport region HTR may further include a compound represented by Formula H-1:

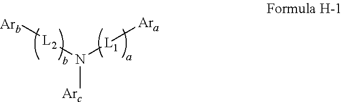

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer of 0 to 10. In one or more embodiments, when a or b is an integer of 2 or greater, a plurality of L₁'s and L₂'s may each independently be a substituted or unsubstituted arylene group having 6 to 30 ring-forming carbon atoms, or a substituted or unsubstituted heteroarylene group having 2 to 30 ring-forming carbon atoms.

In Formula H-1, Arₐ and Ar_{b} may each independently be a substituted or unsubstituted aryl group having 6 to 30 ring-forming carbon atoms, or a substituted or unsubstituted heteroaryl group having 2 to 30 ring-forming carbon atoms. In some embodiments, in Formula H-1, Ar_{c} may be a substituted or unsubstituted aryl group having 6 to 30 ring-forming carbon atoms, or a substituted or unsubstituted heteroaryl group having 2 to 30 ring-forming carbon atoms.

The compound represented by Formula H-1 may be a monoamine compound. In one or more embodiments, the compound represented by Formula H-1 may be a diamine compound in which at least one selected from among Arₐ to Ar_{c} includes the amine group as a substituent. In some embodiments, the compound represented by Formula H-1 may be a carbazole-based compound including a substituted or unsubstituted carbazole group in at least one of Arₐ or Ar_{b}, or a fluorene-based compound including a substituted or unsubstituted fluorene group in at least one of Ar₁ or Ar₂.

The compound represented by Formula H-1 may be represented by any one selected from among the compounds in Compound Group H. However, the compounds listed in Compound Group H are examples, and the compounds represented by Formula H-1 are not limited to those represented by Compound Group H:

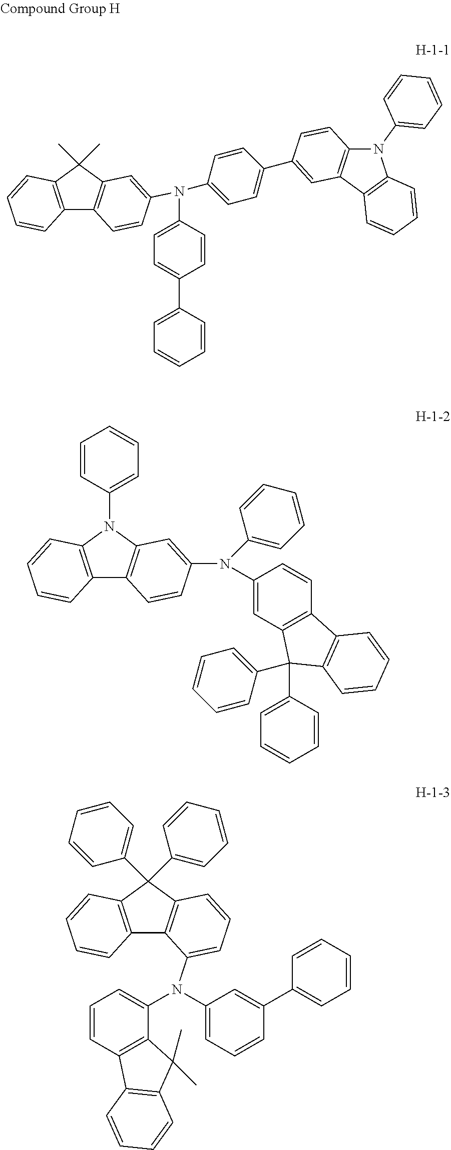 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

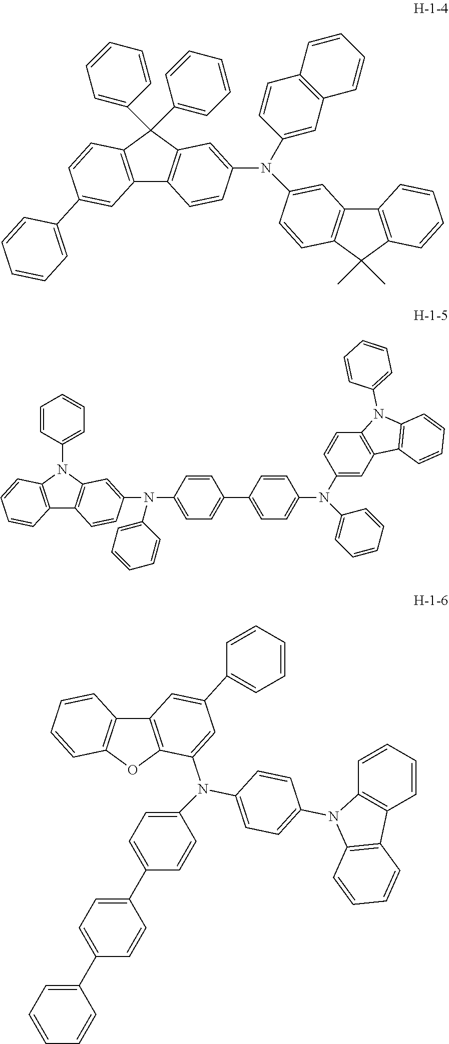 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

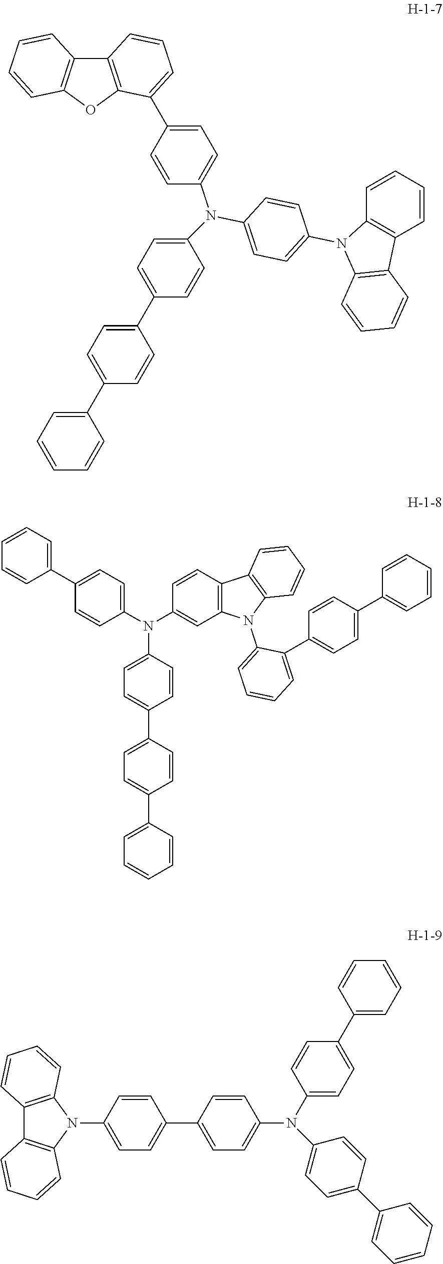 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

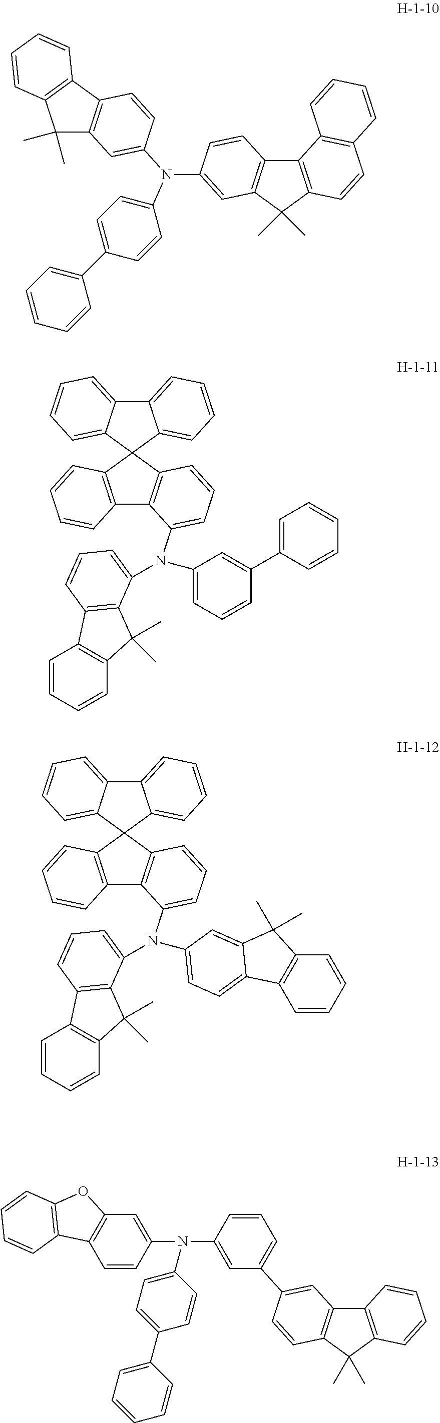 [SMILES: CC1(C)c2ccccc2-c2ccc(N(c3ccc(-c4ccccc4)cc3)c3ccc4c(c3)C(C)(C)c3ccc5ccccc5c3-4)cc21 ; CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

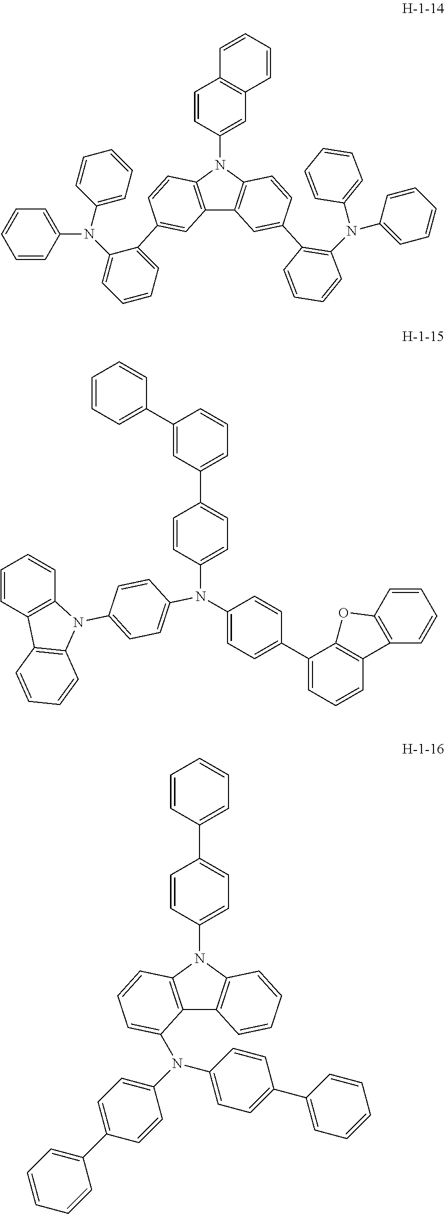 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

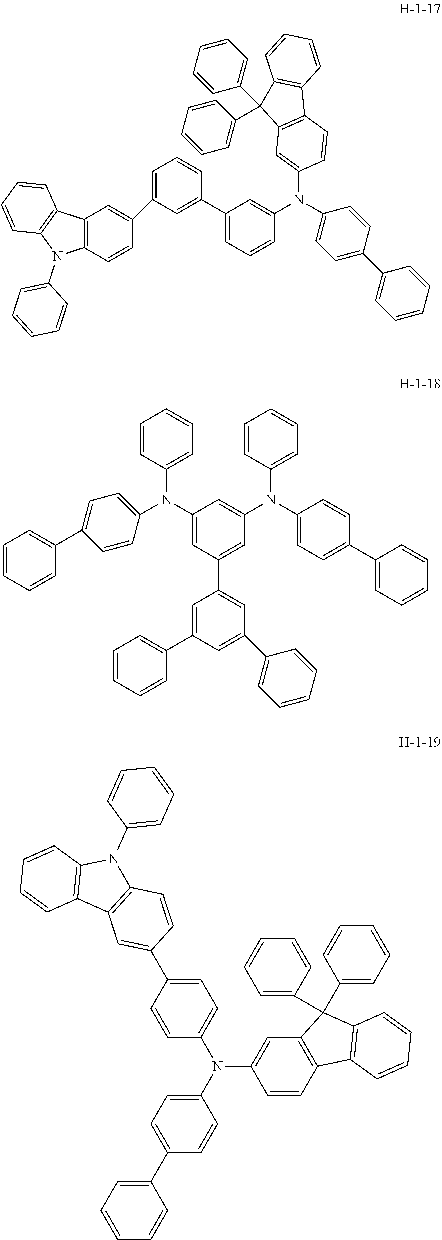 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

In some embodiments, the hole transport region HTR may further include a suitable hole transport material. For example, the hole transport region HTR may include at least one selected from among a phthalocyanine compound such as copper phthalocyanine; N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (in-MTDATA), 4,4′,4″-tris(N,N-diphenylamino)triphenylamine (TDATA), 4,4′,4″-tris[N-(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), and/or the like.

The hole transport region HTR may include a carbazole-based derivative such as at least one selected from among N-phenyl carbazole or polyvinyl carbazole, a fluorene-based derivative, a triphenylamine-based derivative such as N,N′-bis(3-methylphenyl)-N,N′-diphenyl-[1,1-biphenyl]-4,4′-diamine (TPD) or 4,4′,4″-tris(N-carbazolyl)triphenylamine (TCTA), N,N′-di(naphthalene-1-yl)-N,N′-diphenyl-benzidine (NPB), 4,4′-cyclohexylidene bis[N,N-bis(4-methylphenyl)benzenamine](TAPC), 4,4′-bis[N,N′-(3-tolyl)amino]-3,3′-dimethylbiphenyl (HMTPD), 1,3-bis(N-carbazolyl)benzene (mCP), and/or the like.

In some embodiments, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), and/or the like.

The hole transport region HTR may include the herein-described compounds of the hole transport region HTR in at least one of a hole injection layer HIL, a hole transport layer HTL, or an electron blocking layer EBL.

The thickness of the hole transport region HTR may be from about 100 Å to about 10,000 Å, for example, from about 100 Å to about 5,000 Å. When the hole transport region HTR includes the hole injection layer HIL, the hole injection layer HIL may have, for example, a thickness of about 30 Å to about 1,000 Å. When the hole transport region HTR includes the hole transport layer HTL, the hole transport layer HTL may have a thickness of about 250 Å to about 1,000 Å. For example, if (e.g., when) the hole transport region HTR includes the electron blocking layer EBL, the electron blocking layer EBL may have a thickness of about 10 Å to about 1,000 Å. If the thicknesses of the hole transport region HTR, the hole injection layer HIL, the hole transport layer HTL and the electron blocking layer EBL satisfy the herein-described ranges, satisfactory hole transport properties may be achieved without a substantial increase in driving voltage.

The hole transport region HTR may further include a charge generating material to increase conductivity in addition to the herein-described materials. The charge generating material may be dispersed uniformly (e.g., substantially uniformly) or non-uniformly (e.g., substantially non-uniformly) in the hole transport region HTR. The charge generating material may be, for example, a p-dopant. The p-dopant may include at least one of a halogenated metal compound, a quinone derivative, a metal oxide, or a cyano group-containing compound, but one or more embodiments of the present disclosure is not limited thereto. For example, the p-dopant may include a metal halide compound such as CuI or RbI, a quinone derivative such as tetracyanoquinodimethane (TCNQ) or 2,3,5,6-tetrafluoro-7,7,8,8-tetracyanoquinodimethane (F4-TCNQ), a metal oxide such as tungsten oxide or molybdenum oxide, a cyano group-containing compound such as dipyrazino[2,3-f: 2′,3′-h]quinoxaline-2,3,6,7,10,11-hexacarbonitrile (HATCN) or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), and/or the like, but one or more embodiments of the present disclosure is not limited thereto.

As described herein, the hole transport region HTR may further include at least one of the buffer layer or the electron blocking layer EBL in addition to the hole injection layer HIL and the hole transport layer HTL. The buffer layer may compensate for a resonance distance according to the wavelength of light emitted from the emission layer EML and may thus increase light emission efficiency. A material that may be included in the hole transport region HTR may be utilized as a material to be included in the buffer layer. The electron blocking layer EBL is a layer that serves to prevent or reduce the electron injection from the electron transport region ETR to the hole transport region HTR.

In the light emitting element ED of one or more embodiments, the emission layer EML may be to emit blue light. The light emitting element ED of one or more embodiments may include the amine compound of one or more embodiments in the hole transport region HTR and may show high efficiency and long lifetime characteristics in a blue emission region. However, one or more embodiments of the present disclosure is not limited thereto.

In the light emitting element ED of one or more embodiments, the emission layer EML may include an anthracene derivative, a pyrene derivative, a fluoranthene derivative, a chrysene derivative, a dehydrobenzanthracene derivative, or a triphenylene derivative. For example, the emission layer EML may include the anthracene derivative or the pyrene derivative.

In each light emitting element ED of embodiments illustrated in FIGS. 3 to 6, the emission layer EML may further include a suitable host and dopant besides the herein-described host and dopant, and for example the emission layer EML may include a compound represented by Formula E-1. The compound represented by Formula E-1 may be utilized as a fluorescent host material.

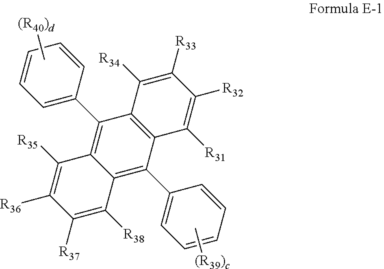

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In one or more embodiments, R₃₁ to R₄₀ may be bonded to an adjacent group to form a saturated hydrocarbon ring or an unsaturated hydrocarbon ring, a saturated heterocycle, or an unsaturated heterocycle.

In Formula E-1, c and d may each independently be an integer of 0 to 5.

Formula E-1 may be represented by any one selected from among Compound E1 to Compound E19:

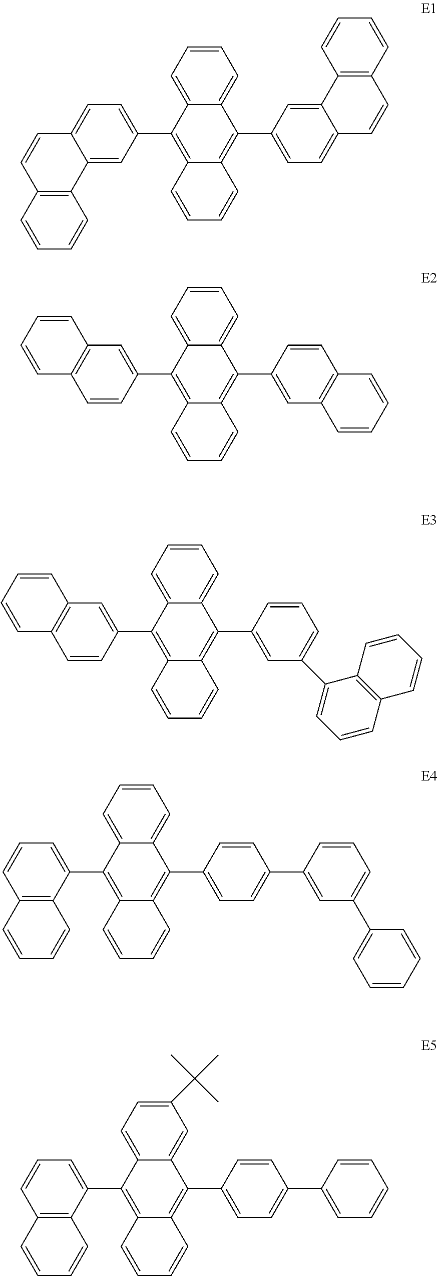

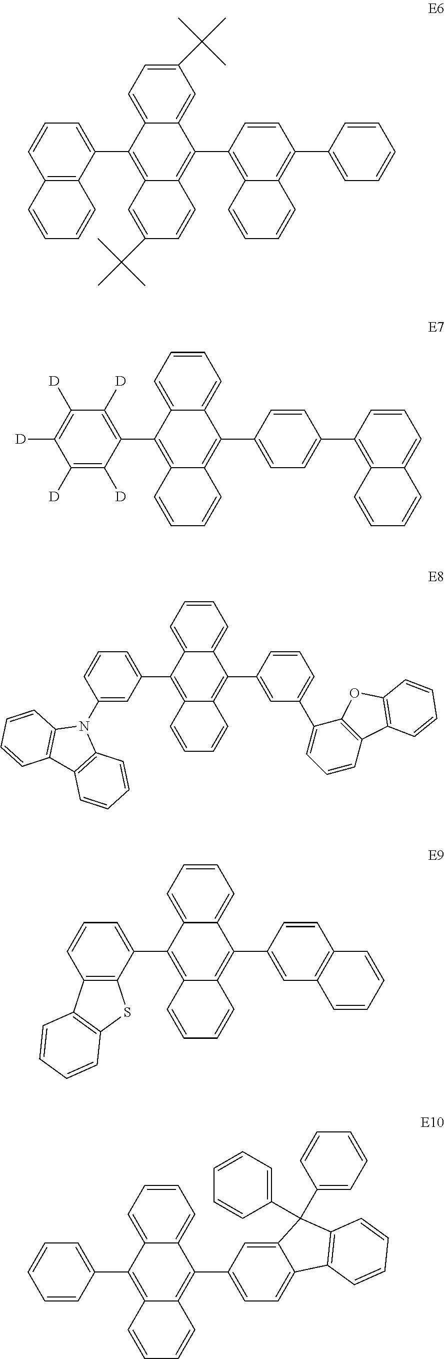

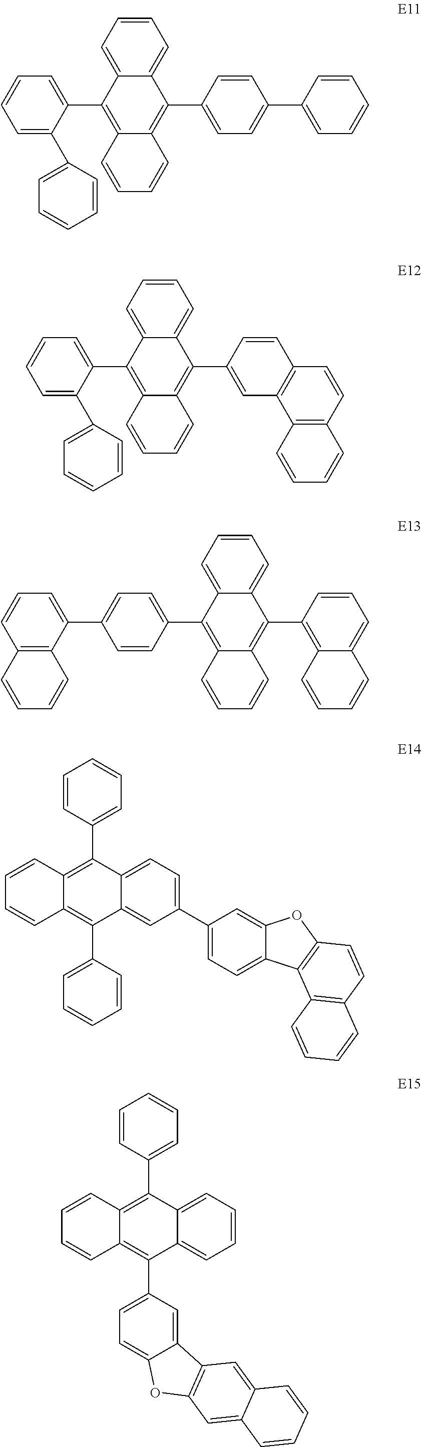

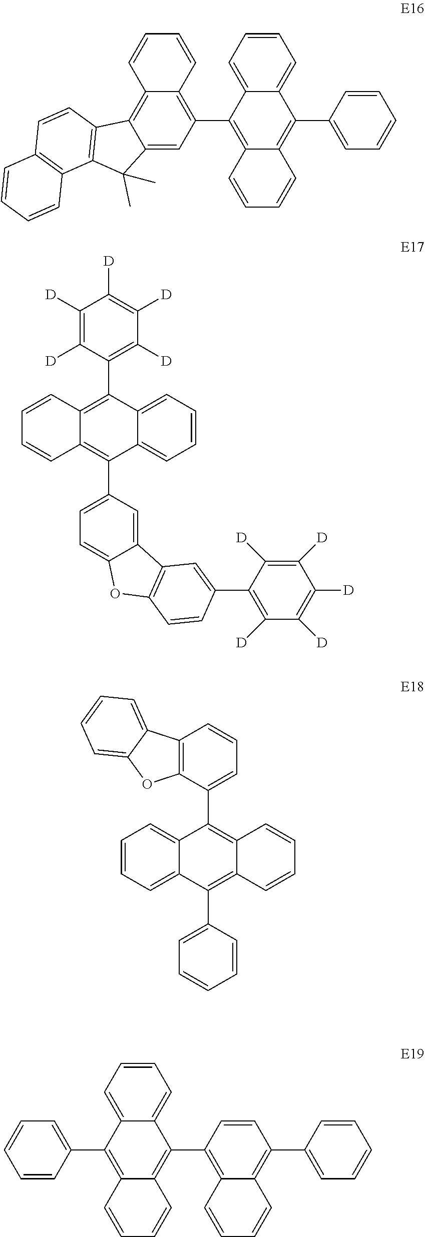

In one or more embodiments, the emission layer EML may include a compound represented by Formula E-2a or Formula E-2b. The compound represented by Formula E-2a or Formula E-2b may be utilized as a phosphorescent host material.

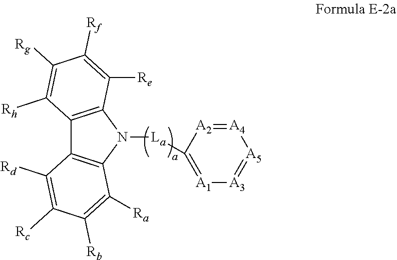

In Formula E-2a, a may be an integer of 0 to 10, and Lₐ may be a direct linkage, a substituted or unsubstituted arylene group having 6 to 30 ring-forming carbon atoms, or a substituted or unsubstituted heteroarylene group having 2 to 30 ring-forming carbon atoms. In one or more embodiments, if (e.g., when) a is an integer of 2 or greater, a plurality of Lₐ's may each independently be a substituted or unsubstituted arylene group having 6 to 30 ring-forming carbon atoms, or a substituted or unsubstituted heteroarylene group having 2 to 30 ring-forming carbon atoms.

In some embodiments, in Formula E-2a, A₁ to A₅ may each independently be N or CRᵢ. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. Rₐ to Rᵢ may be bonded to an adjacent group to form a hydrocarbon ring or a heterocycle containing N, O, S, and/or the like, as a ring-forming atom(s).

In one or more embodiments, in Formula E-2a, two or three selected from among A₁ to A₅ may be N, and the rest may be CRᵢ.

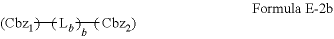

In Formula E-2b, Cbz1 and Cbz2 may each independently be an unsubstituted carbazole group, or a carbazole group substituted with an aryl group having 6 to 30 ring-forming carbon atoms. L_{b} is a direct linkage, a substituted or unsubstituted arylene group having 6 to 30 ring-forming carbon atoms, or a substituted or unsubstituted heteroarylene group having 2 to 30 ring-forming carbon atoms. In one or more embodiments, b is an integer of 0 to 10, and if (e.g., when) b is an integer of 2 or more, a plurality of L_{b}'s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one selected from among the compounds of Compound Group E-2. However, the compounds listed in Compound Group E-2 are merely examples, and the compound represented by Formula E-2a or Formula E-2b is not limited to those represented in Compound Group E-2.

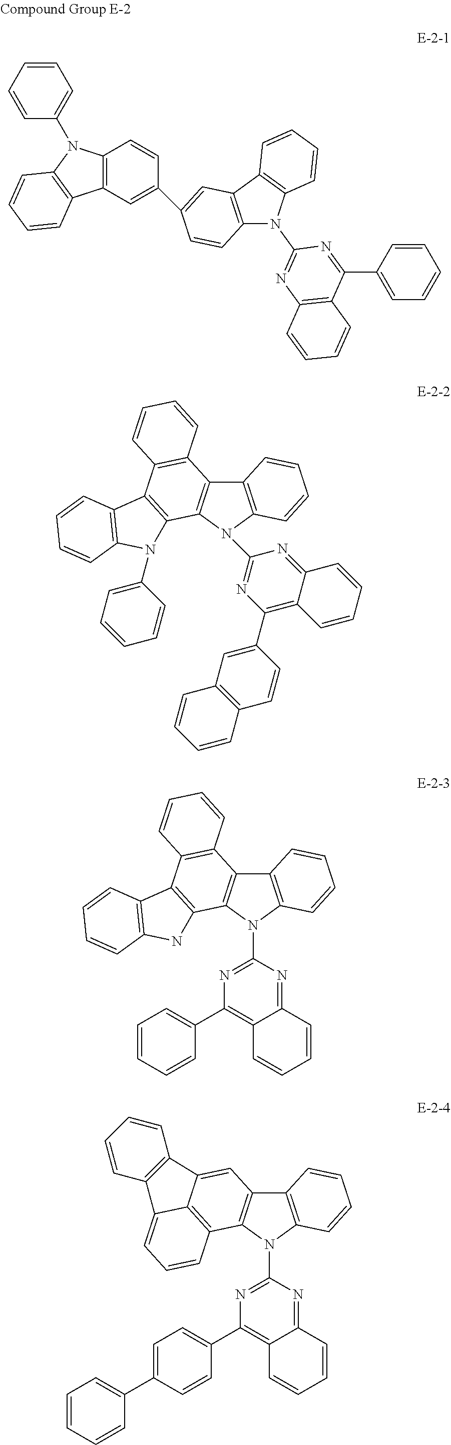

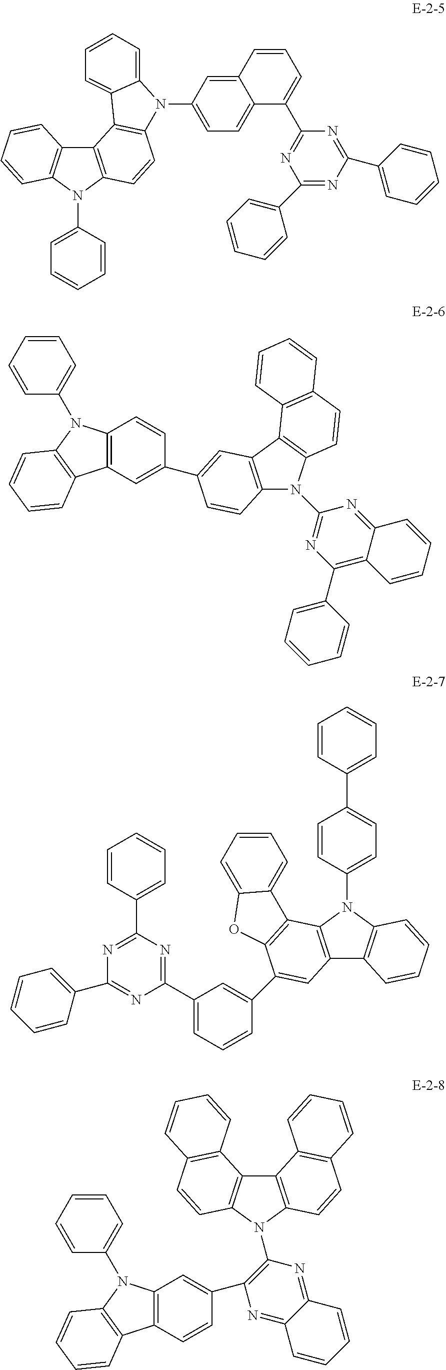 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

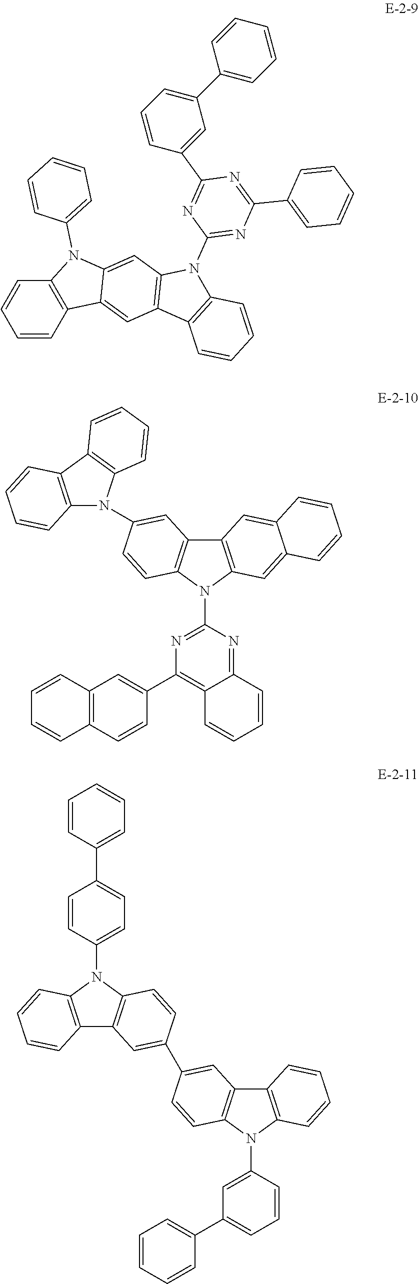 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1]

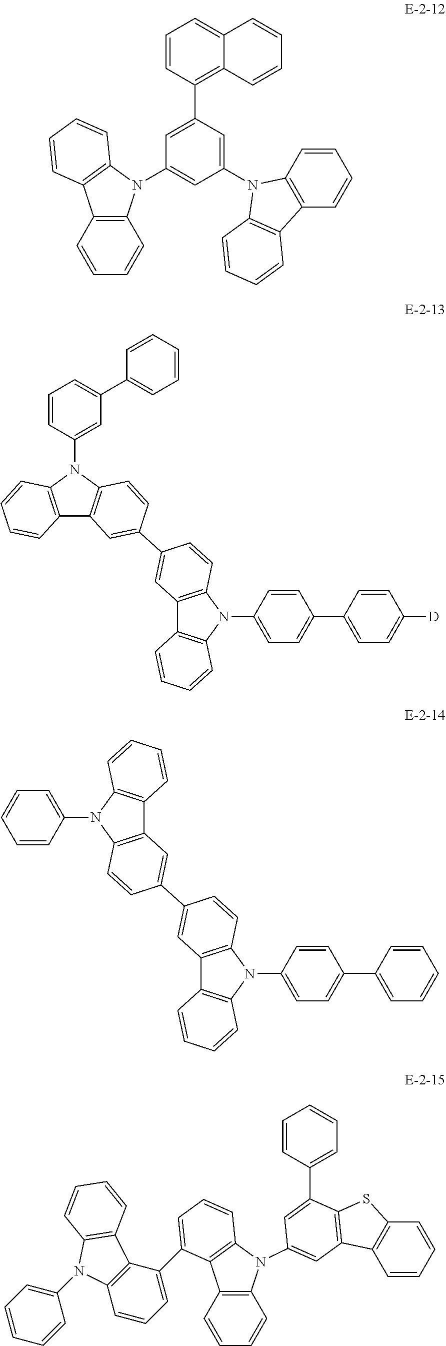 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

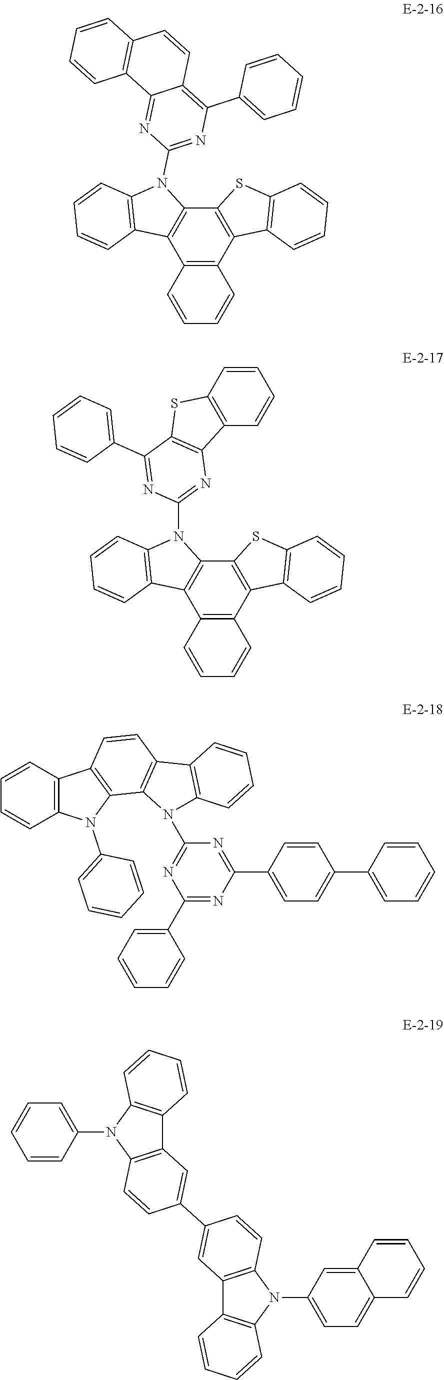

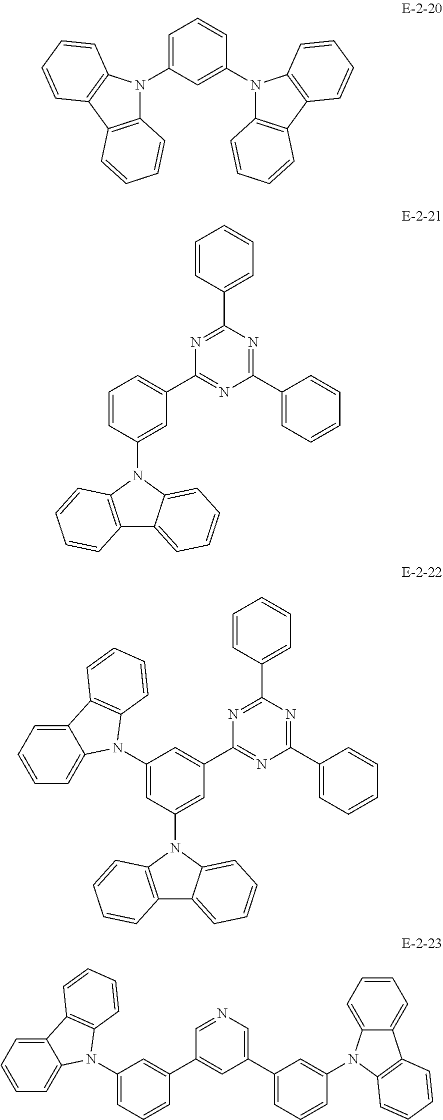 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

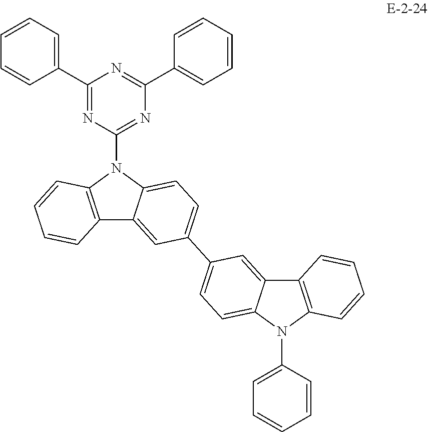 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a general material suitable in the art as a host material. For example, the emission layer EML may include, as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, one or more embodiments of the present disclosure is not limited thereto, for example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetrasiloxane (DPSiO₄), and/or the like may be utilized as a host material.

The emission layer EML may include the compound represented by Formula M-a or M-b. The compound represented by Formula M-a or M-b may be utilized as a phosphorescent dopant material.

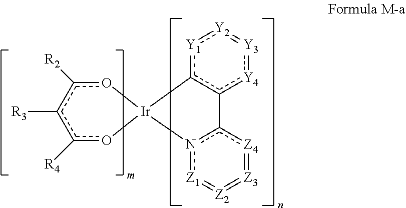

In Formula M-a, Y₁ to Y₄ and Z₁ to Z₄ may each independently be CR₁ or N, R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In Formula M-a, m is 0 or 1, and n is 2 or 3. In Formula M-a, if (e.g., when) m is 0, n is 3, and if (e.g., when) m is 1, n is 2.

The compound represented by Formula M-a may be utilized as a phosphorescent dopant.

The compound represented by Formula M-a may be represented by any one selected from among Compound M-a1 to Compound M-a25. However, Compounds M-a1 to M-a25 are examples, and the compound represented by Formula M-a is not limited to those represented by Compounds M-a1 to M-a25.

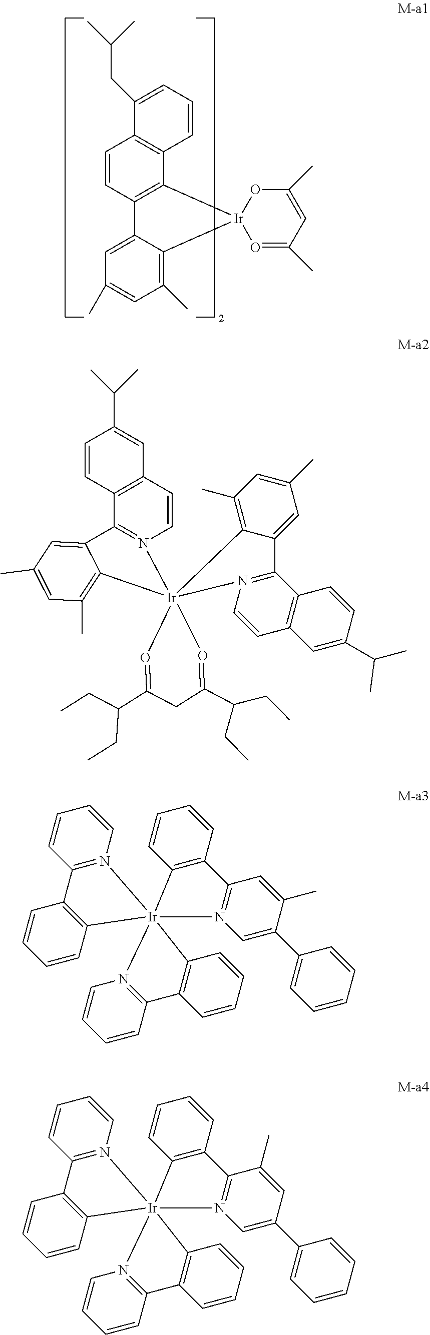

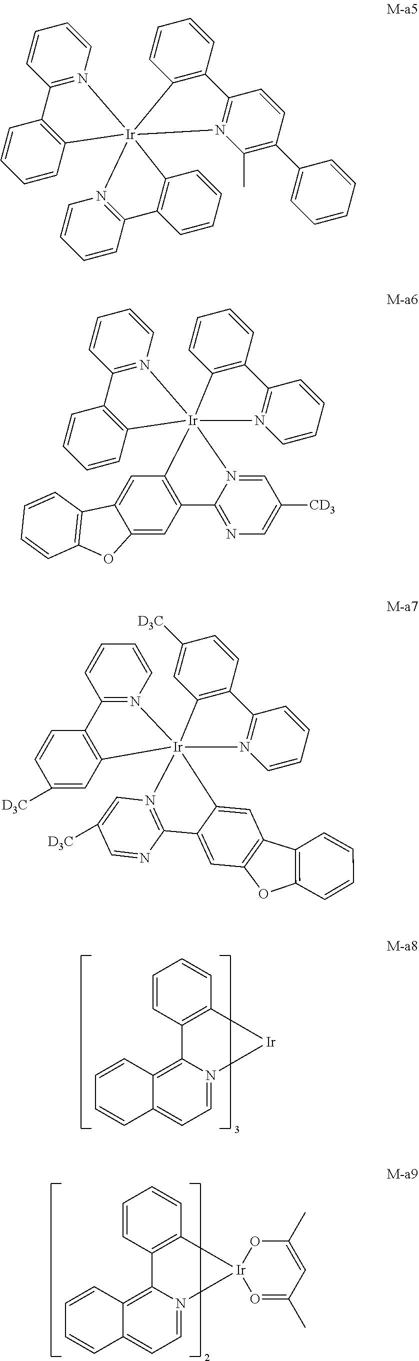

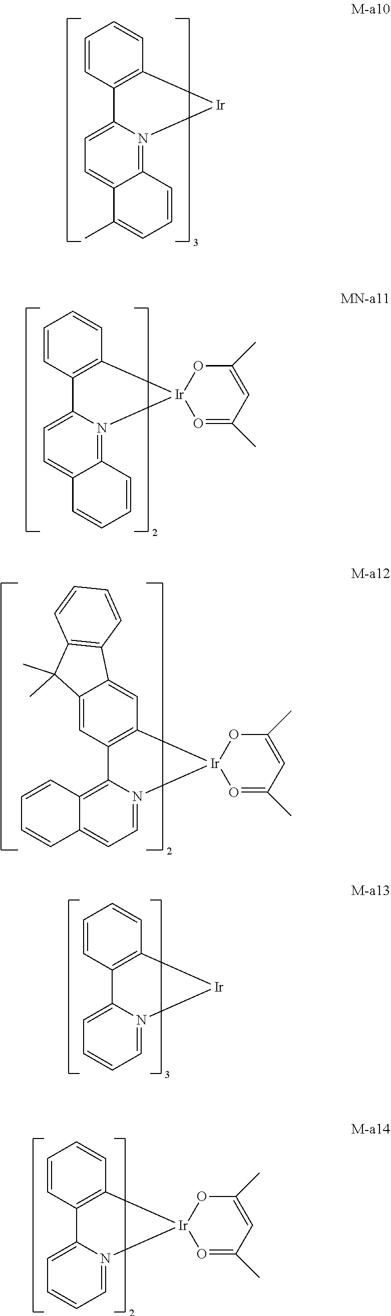

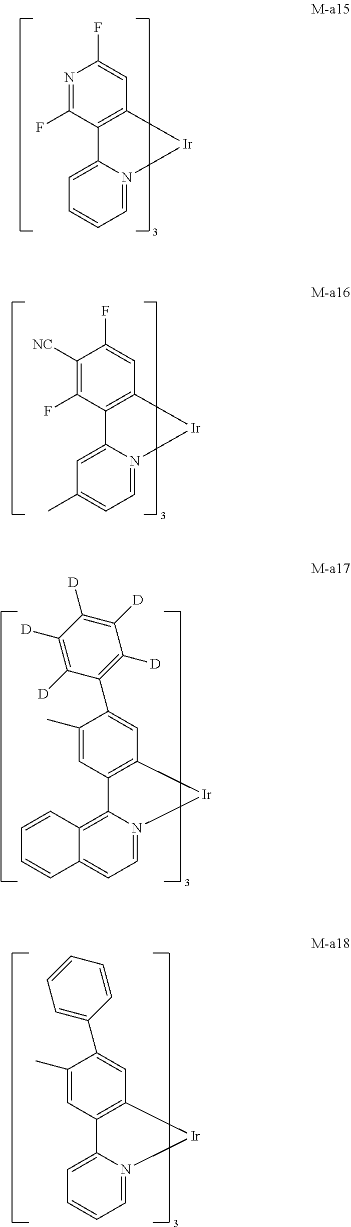

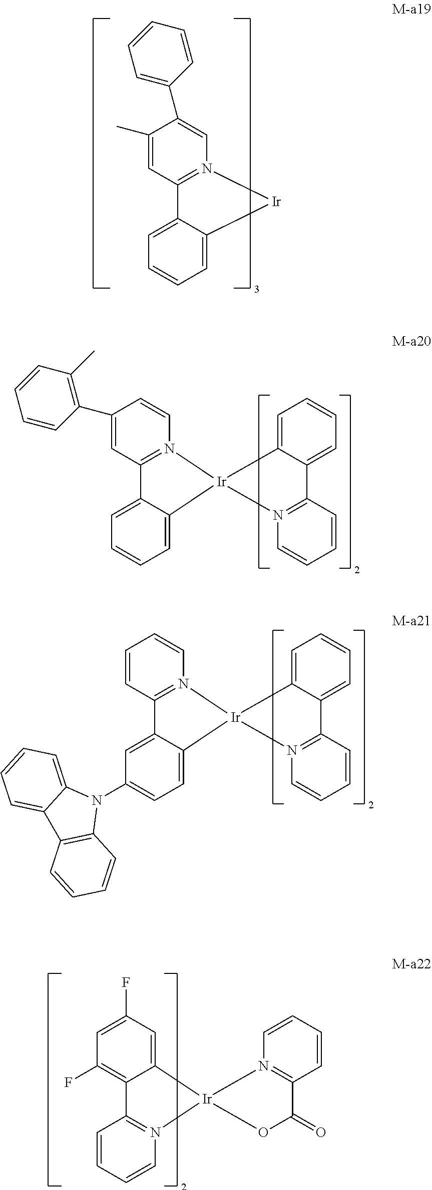

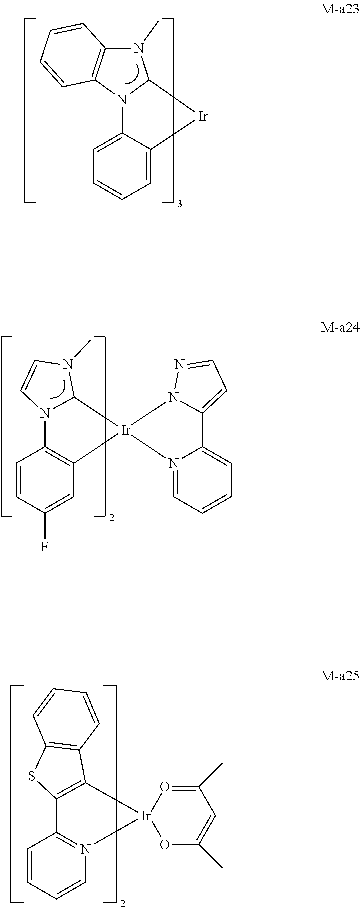

The compound M-a1 and the compound M-a2 may be utilized as a red dopant material, and the compound M-a3 to the compound M-a7 may be utilized as a green dopant material.

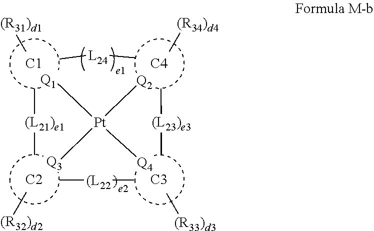

In Formula M-b, Q₁ to Q₄ may each independently be C or N, and C₁ to C₄ may each independently be a substituted or unsubstituted hydrocarbon ring having 5 to ring-forming carbon atoms, or a substituted or unsubstituted hetero ring having 2 to ring-forming carbon atoms. L₂₁ to L₂₄ may each independently be a direct linkage,

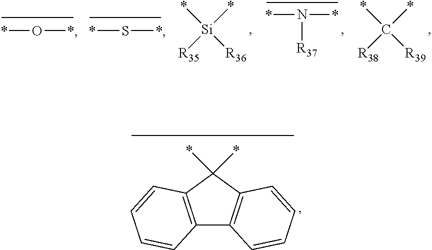

substituted or unsubstituted alkylene group having 1 to 20 carbon, a substituted or unsubstituted arylene group having 6 to 30 ring-forming carbon atoms, or a substituted or unsubstituted heteroarylene group having 2 to 30 ring-forming carbon atoms, and e1 to e4 may each independently be 0 or 1. R₃₁ to R₃₉ may each independently be a hydrogen atom, a deuterium atom, a halogen atom, a cyano group, a substituted or unsubstituted amine group, a substituted or unsubstituted alkyl group having 1 to 20 carbons, a substituted or unsubstituted aryl group having 6 to 30 ring-forming carbon atoms, or a substituted or unsubstituted heteroaryl group having 2 to 30 ring-forming carbon atoms, or forms a ring by being coupled to an adjacent group, and d1 to d4 may each independently be an integer of 0 to 4.

The compound represented by Formula M-b may be utilized as a blue phosphorescent dopant or a green phosphorescent dopant. In some embodiments, the compound represented by Formula M-b may further be included in the light emitting layer EML as an auxiliary dopant in one or more embodiments.

The compound represented by Formula M-b may be represented by any one of compound M-b-1 to compound M-b-11. However, the compounds are only examples, and the compound represented by Formula M-b is not limited to the compound M-b-1 to the compound M-b-11.

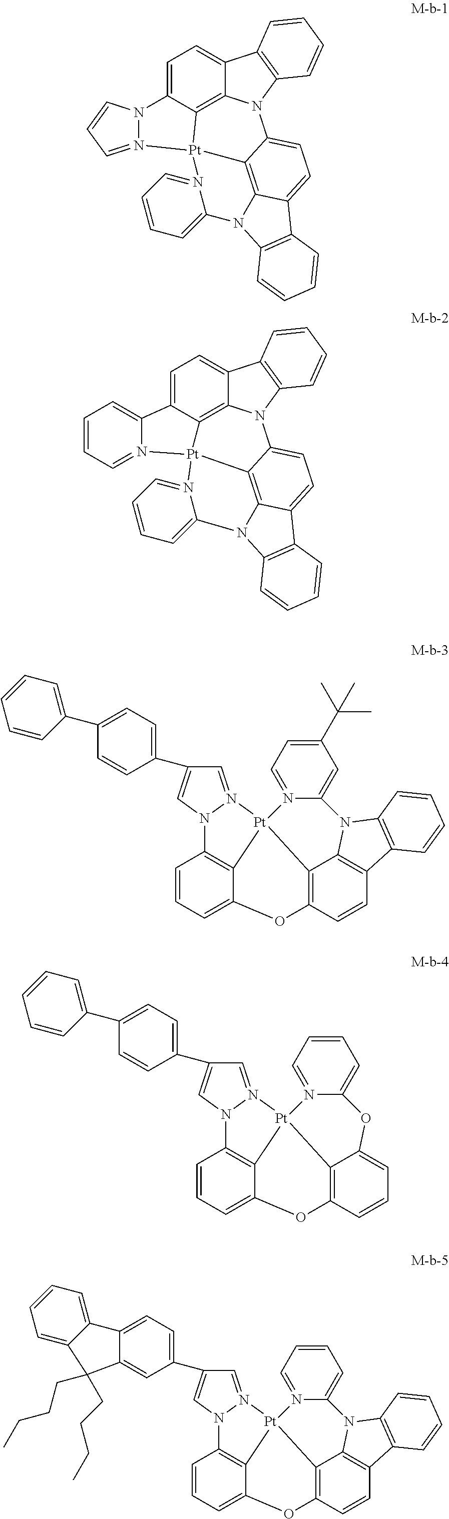

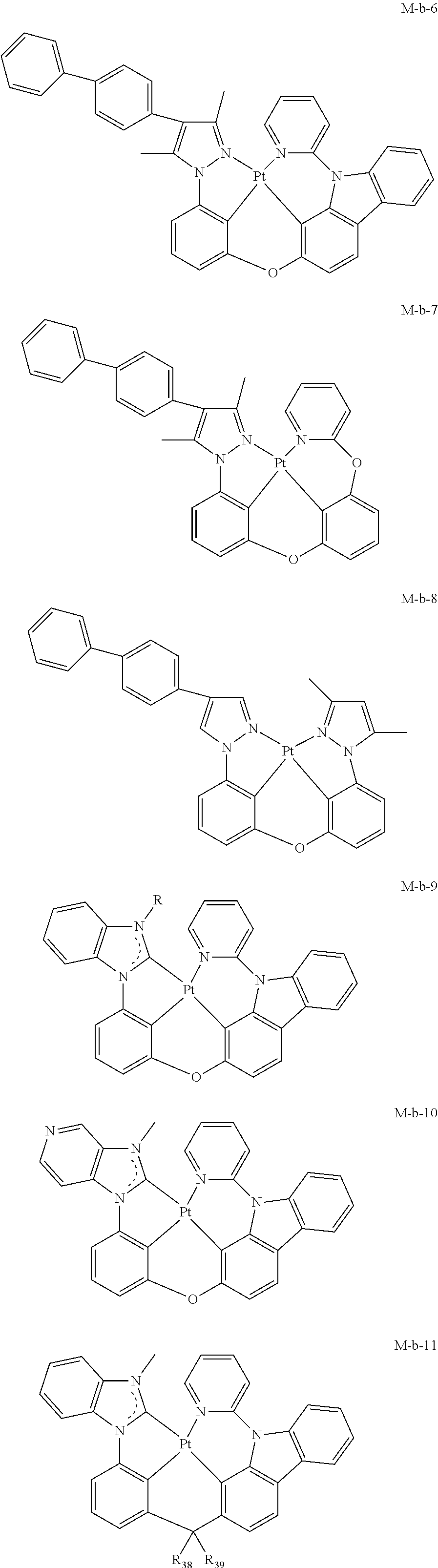

In the compounds described herein, R, R₃₈, and R₃₉ may each independently be a hydrogen atom, a deuterium atom, a halogen atom, a cyano group, a substituted or unsubstituted amine group, a substituted or unsubstituted alkyl group having 1 to 20 carbons, a substituted or unsubstituted aryl group having 6 to 30 ring-forming carbon atoms, or a substituted or unsubstituted heteroaryl group having 2 to 30 ring-forming carbon atoms.

The emission layer EML may include a compound represented by any one selected from among Formula F-a to Formula F-c. The compound represented by Formula F-a to Formula F-c may be utilized as a fluorescence dopant material.

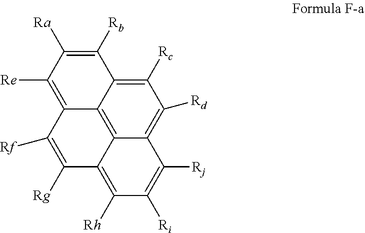

In Formula F-a, at least two selected from among Rₐ to Rⱼ may each independently be substituted with *̅—̅N̅A̅r̅₁̅A̅r̅₂̅. The others, which are not substituted with *̅—̅N̅A̅r̅₁̅A̅r̅₂̅, selected from among Rₐ to Rⱼ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In *̅—̅N̅A̅r̅₁̅A̅r̅₂̅, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one of Ar₁ or Ar₂ may be a heteroaryl group containing O or S as a ring-forming atom.

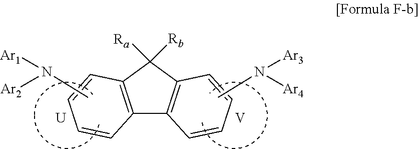

In Formula F-b, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms. At least one selected from among Ar₁ to Ar₄ may be a heteroaryl group containing O or S as a ring-forming atom.

In Formula F-b, the number of rings represented by U and V may each independently be 0 or 1. For example, in Formula F-b, it refers to that if (e.g., when) the number of U or V is 1, one ring constitutes a fused ring at a portion indicated by U or V, and if (e.g., when) the number of U or V is 0, a ring indicated by U or V does not exist. For example, if (e.g., when) the number of U is 0 and the number of V is 1, or if (e.g., when) the number of U is 1 and the number of V is 0, the fused ring having a fluorene core in Formula F-b may be a cyclic compound having four rings. In some embodiments, if (e.g., when) each number of U and V is 0, the fused ring in Formula F-b may be a cyclic compound having three rings. In some embodiments, if (e.g., when) each number of U and V is 1, the fused ring having a fluorene core in Formula F-b may be a cyclic compound having five rings.

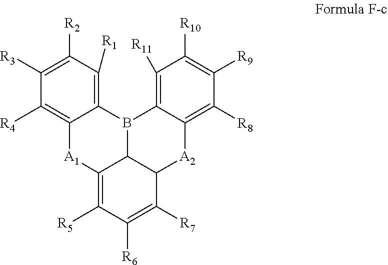

In Formula F-c, A₁ and A₂ may each independently be O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring.

In Formula F-c, A₁ and A₂ may each independently be bonded to substituents of an adjacent ring to form a fused ring. For example, if (e.g., when) A₁ and A₂ are each independently NRₘ, A₁ may be bonded to R₄ or R₅ to form a ring. In some embodiments, A₂ may be bonded to R₇ or R₈ to form a ring.

In one or more embodiments, the emission layer EML may further include, as a suitable dopant material, a styryl derivative (e.g., 1,4-bis[2-(3-N-ethylcarbazolyl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and a derivative thereof (e.g., 2,5,8,11-tetra-t-butylperylene (TBP)), pyrene and a derivative thereof (e.g., 1,1-dipyrene, 1,4-dipyrenylbenzene, 1,4-bis(N,N-diphenylamino)pyrene), and/or the like.

The emission layer EML may further include a suitable phosphorescence dopant material. For example, a metal complex containing iridium (Ir), platinum (Pt), osmium (Os), aurum (Au), titanium (Ti), zirconium (Zr), hafnium (Hf), europium (Eu), terbium (Tb), or thulium (Tm) may be utilized as a phosphorescent dopant. For example, iridium(III) bis(4,6-difluorophenylpyridinato-N,C2) picolinato (Flrpic), bis(2,4-difluorophenylpyridinato)-tetrakis(1-pyrazolyl)borate iridium(III) (FIr6), or platinum octaethyl porphyrin (PtOEP) may be utilized as a phosphorescent dopant. However, one or more embodiments of the disclosure is not limited thereto.

In one or more embodiments, the emission layer EML may include a hole transport host and an electron transport host. In some embodiments, the emission layer EML may include an auxiliary dopant and a light emitting dopant. In one or more embodiments, as the auxiliary dopant, a phosphorescence dopant material or a thermally activated delayed fluorescence dopant material may be included. For example, in one or more embodiments, the emission layer EML may include a hole transport host, an electron transport host, an auxiliary dopant and a light emitting dopant.

In some embodiments, in the emission layer EML, exciplex may be formed by the hole transport host and the electron transport host. In this case, the triplet energy of the exciplex formed by the hole transport host and the electron transport host may correspond to the gap T1 between the LUMO energy level of the electron transport host and the HOMO energy level of the hole transport host.

In one or more embodiments, the triplet energy (T1) of the exciplex formed by the hole transport host and the electron transport host may be about 2.4 electron volt (eV) to about 3.0 eV. In some embodiments, the triplet energy of the exciplex may be a smaller value than the energy gap of each host material. Accordingly, the exciplex may have the triplet energy of about 3.0 eV or less, which is the gap between the hole transport host and the electron transport host.

In one or more embodiments, at least one emission layer EML may include a quantum dot material.

In the description, the quantum dot refers to the crystal of a semiconductor compound. The quantum dot may be to emit light in one or more suitable emission wavelengths according to the size of the crystal. The quantum dot may be to emit light in one or more suitable emission wavelengths by controlling the element ratio in the quantum dot compound. The diameter of the quantum dot may be, for example, about nanometer (nm) to about 10 nm. Unless otherwise defined, in the present disclosure, the term “particle diameter” or “dot diameter” refers to an average diameter if (e.g., when) particles or dots are spherical and refers to an average major axis length if (e.g., when) particles or dots are non-spherical.

In one or more embodiments, the quantum dot may be synthesized by a chemical bath deposition, a metal organic chemical vapor deposition (MOCVD), a molecular beam epitaxy (MBE) or a similar process therewith. For example, the chemical bath deposition is a method of mixing an organic solvent and a precursor material and then, growing a quantum dot particle crystal. During growing the crystal, the organic solvent may naturally play the role of a dispersant which is coordinated on the surface of the quantum dot crystal and may control the growth of the crystal. Accordingly, the chemical bath deposition is more advantageous if (e.g., when) compared to a vapor deposition method including a metal organic chemical vapor deposition (MOCVD) and molecular beam epitaxy (MBE), and the growth of the quantum dot particle may be controlled or selected through a low-cost process.

The core of the quantum dot may be selected from among a Group II-VI compound, a Group III-VI compound, a Group I-III-VI compound, a Group III-V compound, a Group III-II-V compound, a Group IV-VI compound, a Group IV element, a Group IV compound, a Group II-IV-V compound, and/or a (e.g., any suitable) combination thereof.

The Group II-VI compound may be selected from among the group including (e.g., consisting of) a binary compound selected from among the group including (e.g., consisting of) CdSe, CdTe, CdS, ZnS, ZnSe, ZnTe, ZnO, HgS, HgSe, HgTe, MgSe, MgS, and/or a (e.g., any suitable) mixture thereof, a ternary compound selected from among the group including (e.g., consisting of) CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and/or a (e.g., any suitable) mixture thereof, and a quaternary compound selected from among the group including (e.g., consisting of) HgZnTeS, CdZnSeS, CdZnSeTe, CdZnSTe, CdHgSeS, CdHgSeTe, CdHgSTe, HgZnSeS, HgZnSeTe, and/or a (e.g., any suitable) mixture thereof. In one or more embodiments, the Group II-VI semiconductor compound may further include a Group I metal and/or a Group IV element. The Group I-II-VI compound may be selected from among CuSnS or CuZnS, and the Group II-IV-VI compound may be selected from among ZnSnS and/or the like. Group I-II-IV-VI compounds may be selected from among tetraelement compounds selected from among the group including (e.g., consisting of) Cu₂ZnSnS₂, Cu₂ZnSnS₄, Cu₂ZnSnSe₄, Ag₂ZnSnS₂, and mixtures thereof.

The Group III-VI compound may include a binary compound such as GaS, Ga₂S₃, GaSe, Ga₂Se₃, GaTe, InS, InSe, In₂Se₃, InTe, In₂S₃, a ternary compound such as InGaS₃ or InGaSes, or any combination thereof.

The Group I-III-VI compound may be selected from among a ternary compound selected from among the group including (e.g., consisting of) AgInS, AgInS₂, CuInS, CuInS₂, AgGaS₂, CuGaS₂, CuGaO₂, AgGaO₂, AgAlO₂, AgInSe₂, AgGaS, AgGaSe₂, CuInSe₂, CuGaSe₂, and/or a (e.g., any suitable) mixture thereof, or a quaternary compound such as AgInGaS₂, AgInGaSe₂, CuInGaS₂.

The Group III-V compound may be selected from among the group including (e.g., consisting of) a binary compound selected from among the group including (e.g., consisting of) GaN, GaP, GaAs, GaSb, AlN, AlP, AlAs, AlSb, InN, InP, InAs, InSb, and/or a (e.g., any suitable) mixture thereof, a ternary compound selected from among the group including (e.g., consisting of) GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and/or a (e.g., any suitable) mixture thereof, and a quaternary compound selected from among the group including (e.g., consisting of) GaAINP, GaAINAs, GaAINSb, GaAlPAs, GaAlPSb, GaInNP, GaInNAs, GalnNSb, GaInPAs, GalnPSb, InAINP, InAINAs, InAINSb, InAlPAs, InAlPSb, and/or a (e.g., any suitable) mixture thereof. In one or more embodiments, the Group III-V compound may further include a Group II metal. For example, InZnP, InGaZnP, InAIZnP, and/or the like, may be selected as a Group III-II-V compound.

The Group IV-VI compound may be selected from among the group including (e.g., consisting of) a binary compound selected from among the group including (e.g., consisting of) SnS, SnSe, SnTe, PbS, PbSe, PbTe, and/or a (e.g., any suitable) mixture thereof, a ternary compound selected from among the group including (e.g., consisting of) SnSeS, SnSeTe, SnSTe, PbSeS, PbSeTe, PbSTe, SnPbS, SnPbSe, SnPbTe, and/or a (e.g., any suitable) mixture thereof, and a quaternary compound selected from among the group including (e.g., consisting of) SnPbSSe, SnPbSeTe, SnPbSTe, and/or a (e.g., any suitable) mixture thereof. The Group IV element may be selected from among the group including (e.g., consisting of) Si, Ge, and/or a (e.g., any suitable) mixture thereof. The Group IV compound may be a binary compound selected from among the group including (e.g., consisting of) SiC, SiGe, and/or a (e.g., any suitable) mixture thereof.

The Group II-IV-V compound may be selected from among a ternary compound selected from among the group including (e.g., consisting of) ZnSnP, ZnSnP₂, ZnSnAs₂, ZnGeP₂, ZnGeAs₂, CdSnP₂, or CdGeP₂, and/or a (e.g., any suitable) mixture thereof.

Each element included in a polynary compound such as the binary compound, the ternary compound, or the quaternary compound may be present in a particle with a substantially uniform or substantially non-uniform concentration distribution. For example, the formulae refer to the types (kinds) of elements included in the compounds, and the elemental ratio in the compound may be different. For example, AgInGaS₂ may refer to AgInₓGa₁₋ₓS₂ (where x is a real number of 0 to 1).

Additionally, the quantum dot of one or more embodiments may have a core-shell structure in which one quantum dot surrounds another quantum dot. In core-shell structure, an interface between the core and the shell may have a concentration gradient in which the concentration of an element present in the shell becomes lower towards the center.

In one or more embodiments, the quantum dot may have the herein-described core/shell structure including a core containing nanocrystals and a shell around (e.g., surrounding) the core. The shell of the quantum dot may serve as a protection layer to prevent or reduce the chemical deformation of the core to maintain semiconductor properties, and/or a charging layer to impart electrophoresis properties to the quantum dot. The shell may be a single layer or multiple layers. An example of the shell of the quantum dots may include a metal or non-metal oxide, a semiconductor compound, and/or a (e.g., any suitable) combination thereof.

For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO, or a ternary compound such as MgAl₂O₄, CoFe₂O₄, NiFe₂O₄, or CoMn₂O₄, but one or more embodiments of the present disclosure is not limited thereto.

Also, examples of the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, and/or the like, but one or more embodiments of the present disclosure is not limited thereto.

The quantum dot may have a full width of half maximum (FWHM) of a light emitting wavelength spectrum of about 45 nm or less, about 40 nm or less, or about 30 nm or less, and color purity or color reproducibility may be improved in the herein-described ranges. In some embodiments, light emitted through such quantum dot is emitted in all directions so that a wide viewing angle may be improved.

In some embodiments, although the form of the quantum dot is not particularly limited as long as it is a form commonly utilized in the art, more specifically, the quantum dot in the form of spherical, pyramidal, multi-arm, or cubic nanoparticles, nanotubes, nanowires, nanofibers, nanoplate particles, and/or the like may be utilized.

The quantum dot may control the color of light emitted according to the size of the particle, and accordingly, the quantum dot may have one or more suitable emission colors such as blue, red, and green.

As the size of the quantum dot is adjusted or the elemental ratio in the quantum dot compound is adjusted, it is possible to control the energy band gap, and thus light in one or more suitable wavelength ranges may be obtained in the quantum dot emission layer. Therefore, the quantum dot as described herein (utilizing different sizes of quantum dots or different elemental ratios in the quantum dot compound) is utilized, and thus the light emitting element, which emits light in one or more suitable wavelengths, may be implemented. For example, the adjustment of the size of the quantum dot or the elemental ratio in the quantum dot compound may be selected to emit red, green, and/or blue light. In some embodiments, the quantum dots may be configured to emit white light by combining one or more suitable colors of light.

In each of the light emitting elements ED of embodiments illustrated in FIGS. 3 to 6, the electron transport region ETR is provided on the emission layer EML. The electron transport region ETR may include at least one of the hole blocking layer HBL, the electron transport layer ETL, or the electron injection layer EIL, but one or more embodiments of the present disclosure is not limited thereto.

The electron transport region ETR may have a single layer formed of a single material, a single layer formed of a plurality of different materials, or a multilayer structure including a plurality of layers formed of a plurality of different materials.

For example, the electron transport region ETR may have a single layer structure of the electron injection layer EIL or the electron transport layer ETL, and may have a single layer structure formed of an electron injection material and an electron transport material. In some embodiments, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL are stacked in order from the emission layer EML, but one or more embodiments of the present disclosure is not limited thereto. The electron transport region ETR may have a thickness, for example, from about 1,000 angstrom (A) to about 1,500 Å.

The electron transport region ETR may be formed utilizing one or more suitable methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include at least one compound represented by Formula ET-1:

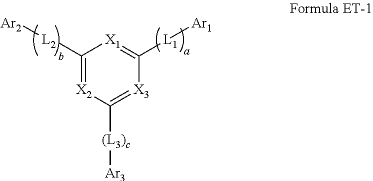

In Formula ET-1, at least one selected from among X₁ to X₃ is N, and the rest are CRₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may each independently be an integer of 0 to 10. In Formula ET-1, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In one or more embodiments, if (e.g., when) a to c may each independently be an integer of 2 or more, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, one or more embodiments of the present disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl) phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), beryllium bis(benzoquinolin-10-olate) (Bebq₂), 9,10-di(naphthalene-2-yl)anthracene (ADN), 1,3-bis[3,5-di(pyridin-3-yl)phenyl]benzene (BmPyPhB), and/or a (e.g., any suitable) mixture thereof.

The electron transport region ETR may include at least one selected from among Compound ET1 to Compound ET36:

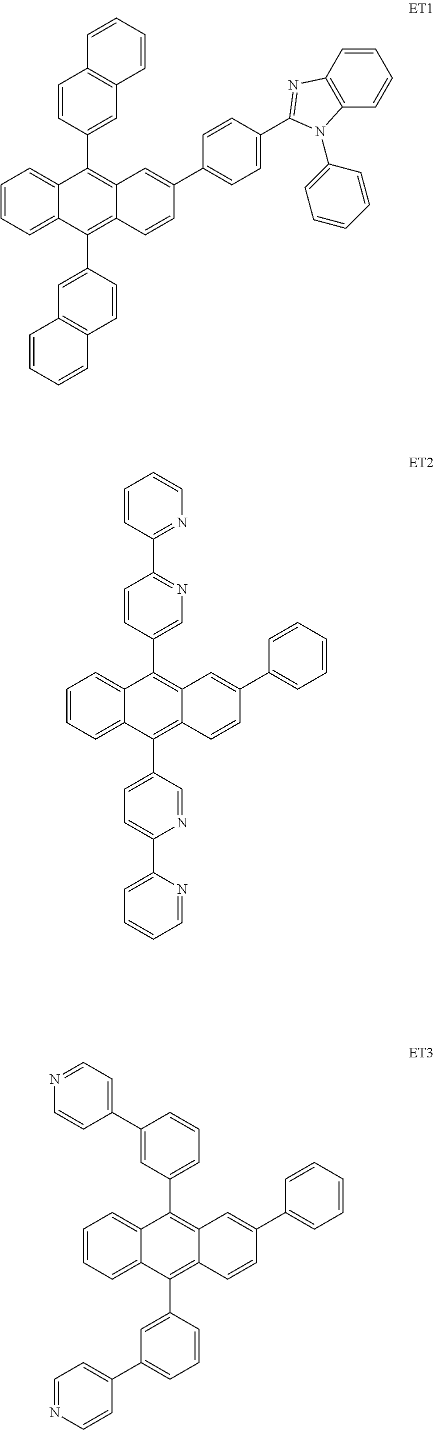 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

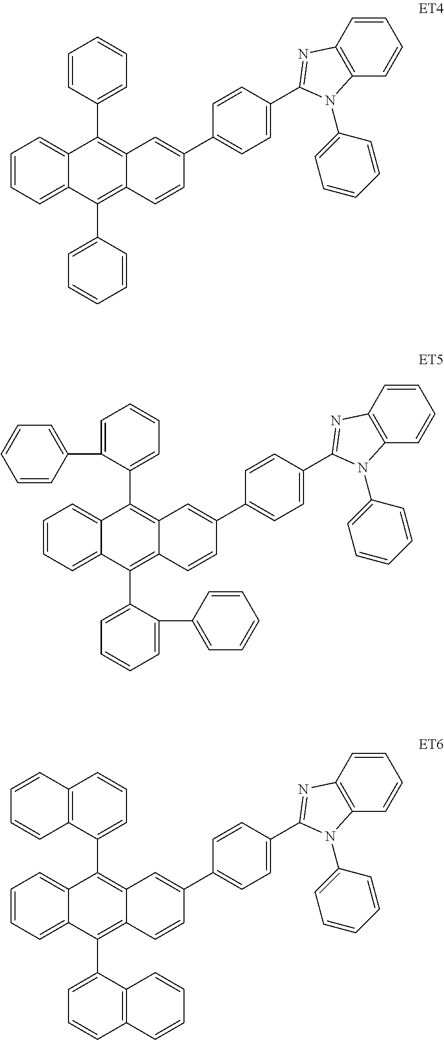 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

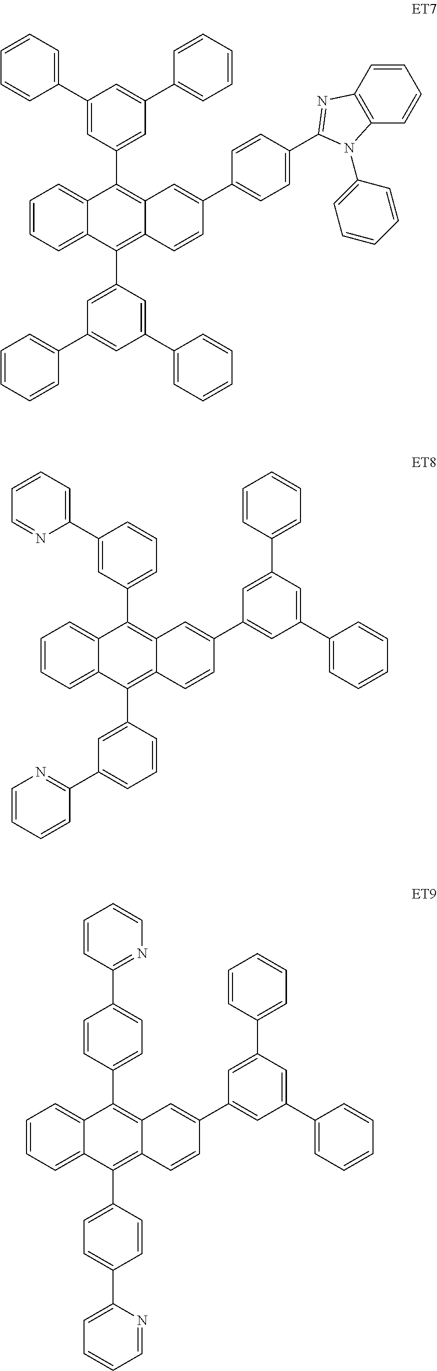 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

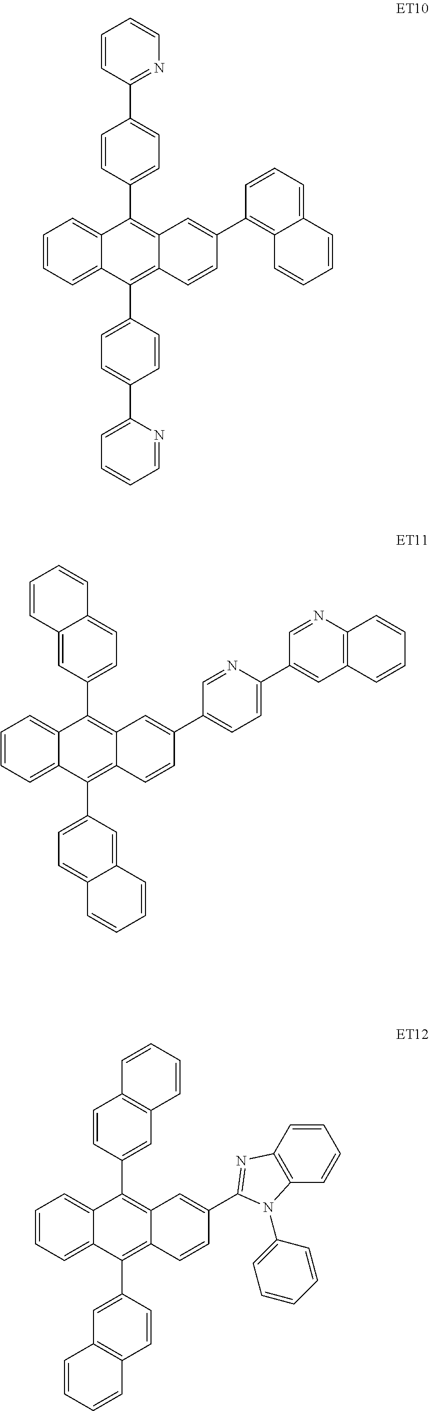 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

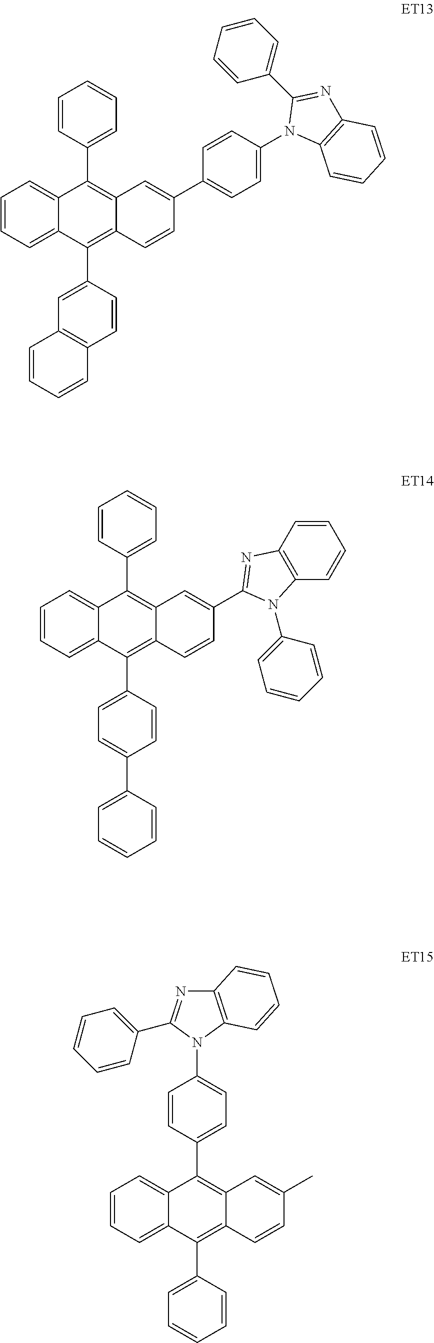 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

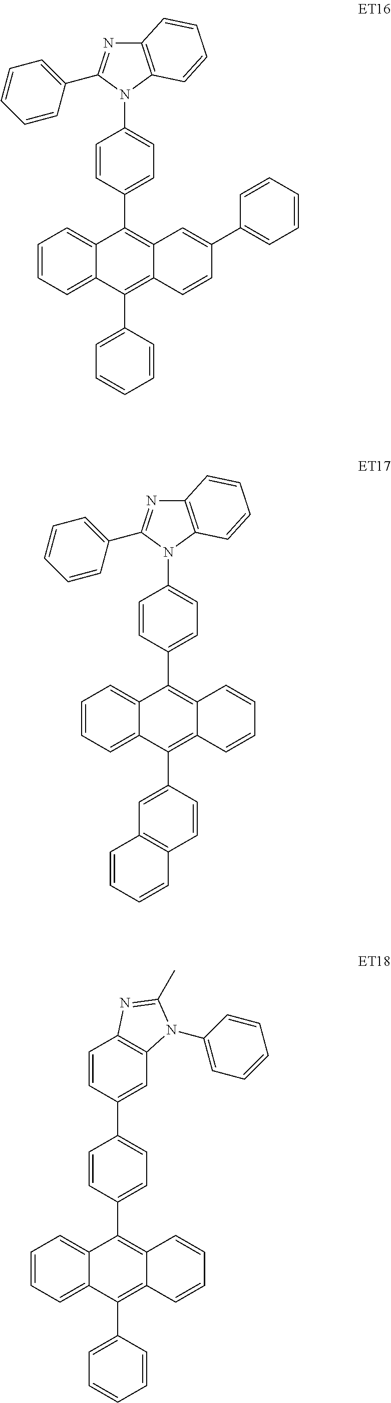 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

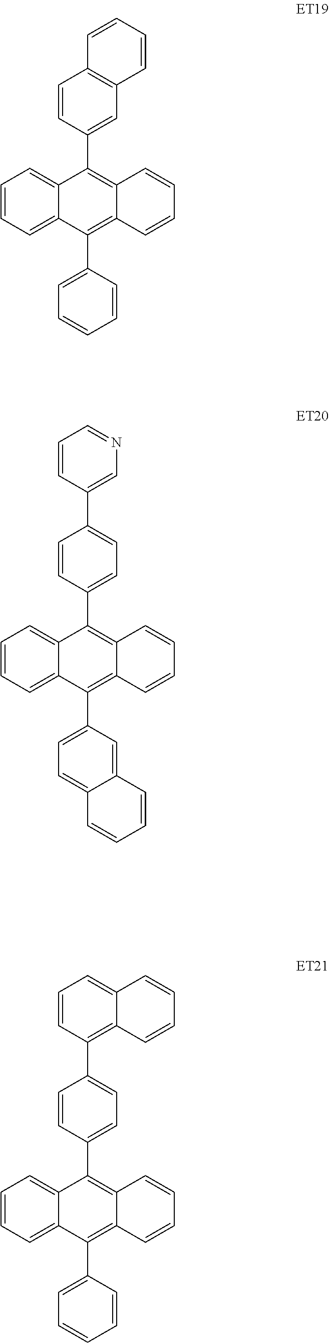

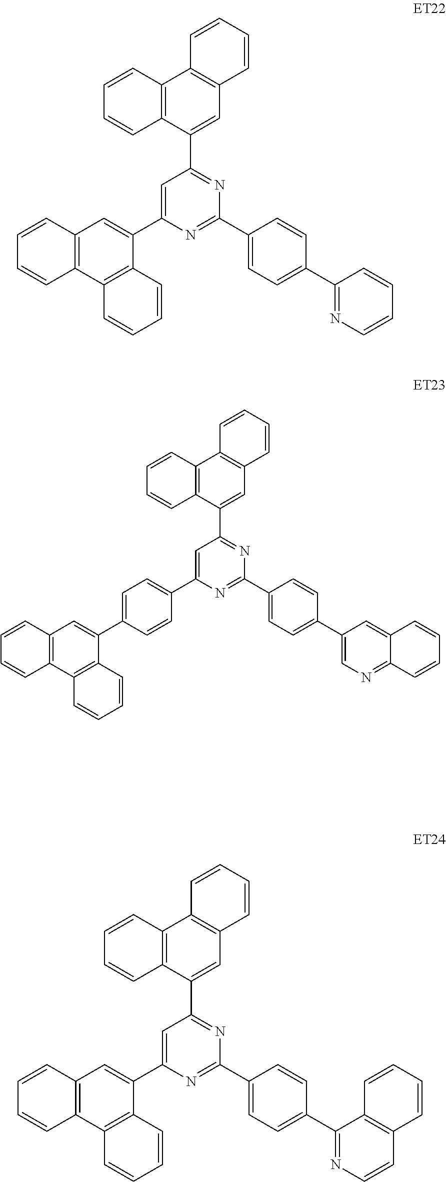 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

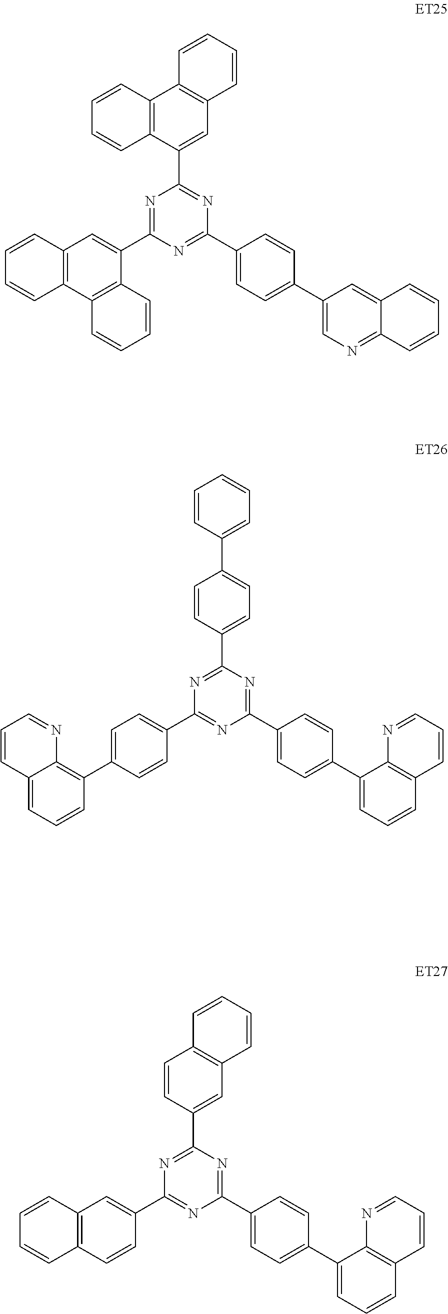 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

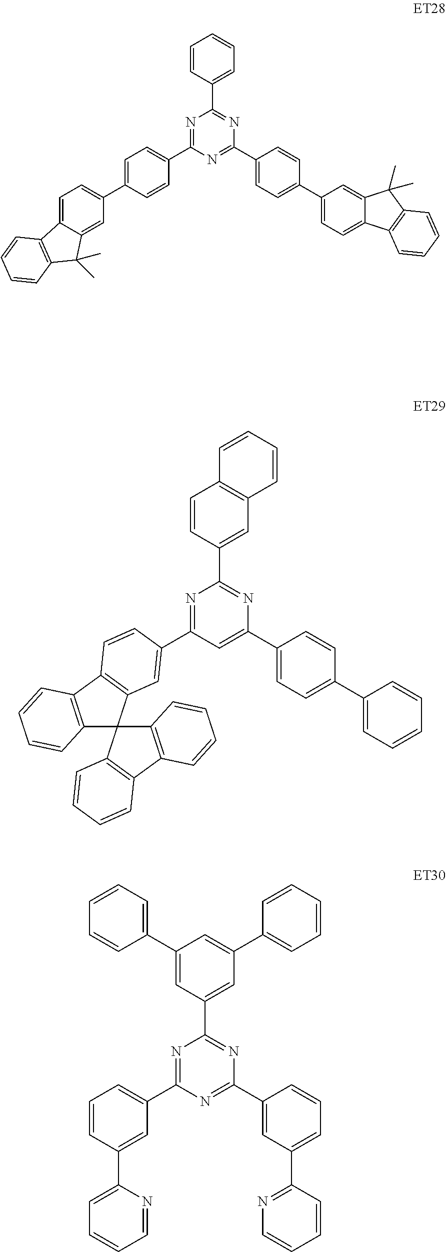 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

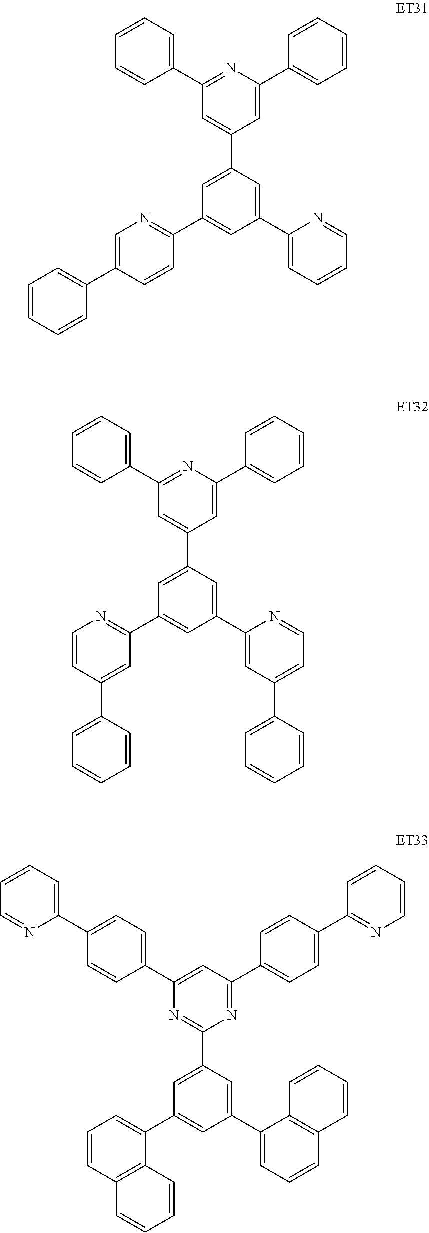 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

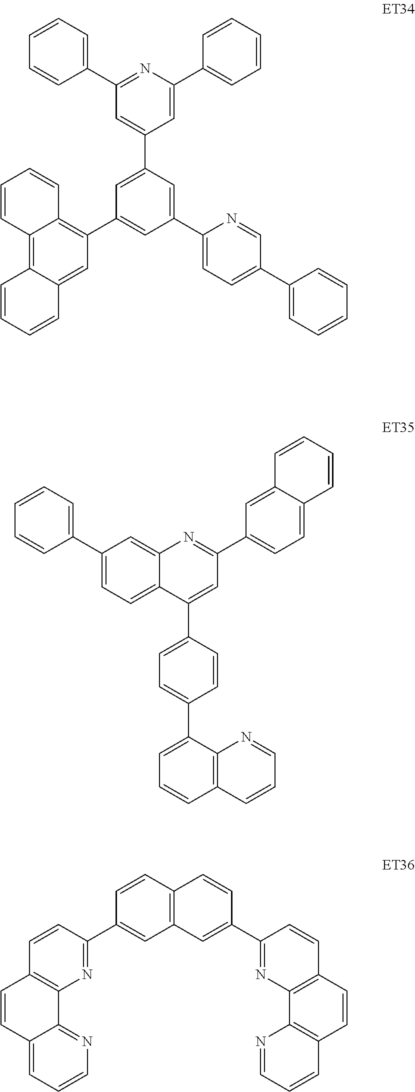 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport region ETR may include a metal halide such as LiF, NaCl, CsF, RbCl, RbI, CuI, and KI, a lanthanide metal such as Yb, and a co-deposited material of the metal halide and the lanthanide metal. For example, the electron transport region ETR may include KI:Yb, RbI:Yb, LiF:Yb, and/or the like, as a co-deposited material. In one or more embodiments, the electron transport region ETR may be formed utilizing a metal oxide such as Li₂O or BaO, or 8-hydroxyl-lithium quinolate (Liq), and/or the like, but one or more embodiments of the present disclosure is not limited thereto. The electron transport region ETR may also be formed of a mixture material of an electron transport material and an insulating organometallic salt. The organometallic salt may be a material having an energy band gap of about 4 eV or more. For example, the organometallic salt may include, for example, a metal acetate, a metal benzoate, a metal acetoacetate, a metal acetylacetonate, a metal stearate, and/or the like.

The electron transport region ETR may further include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to the herein-described materials, but one or more embodiments of the present disclosure is not limited thereto.

The electron transport region ETR may include the herein-described compounds of the electron transport ETR region in at least one of the electron injection layer EIL, the electron transport layer ETL, or the hole blocking layer HBL.

When the electron transport region ETR includes the electron transport layer ETL, the electron transport layer ETL may have a thickness of about 100 Å to about 1,000 Å, for example, about 150 Å to about 500 Å. If the thickness of the electron transport layer ETL satisfies the aforementioned ranges, satisfactory electron transport characteristics may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. If the thickness of the electron injection layer EIL satisfies the herein-described ranges, satisfactory electron injection characteristics may be obtained without a substantial increase in driving voltage.

The second electrode EL2 is provided on the electron transport region ETR. The second electrode EL2 may be a common electrode. The second electrode EL2 may be a cathode or an anode, but one or more embodiments of the present disclosure is not limited thereto. For example, if (e.g., when) the first electrode EL1 is an anode, the second electrode EL2 may be a cathode, and if (e.g., when) the first electrode EL1 is a cathode, the second electrode EL2 may be an anode.

The second electrode EL2 may be a transmissive electrode, a transflective electrode, or a reflective electrode. When the second electrode EL2 is the transmissive electrode, the second electrode EL2 may be formed of a transparent metal oxide, for example, indium tin oxide (ITO), indium zinc oxide (IZO), zinc oxide (ZnO), indium tin zinc oxide (ITZO), and/or the like.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, or a compound or mixture thereof (e.g., AgMg, AgYb, or MgAg). In one or more embodiments, the second electrode EL2 may have a multilayer structure including a reflective film or a transflective film formed of the herein-described materials, and a transparent conductive film formed of ITO, IZO, ZnO, ITZO, and/or the like. For example, the second electrode EL2 may include the herein-described metal materials, combinations of at least two metal materials of the herein-described metal materials, oxides of the herein-described metal materials, and/or the like.

In one or more embodiments, the second electrode EL2 may be connected with an auxiliary electrode. If the second electrode EL2 is connected with the auxiliary electrode, the resistance of the second electrode EL2 may be decreased.

In one or more embodiments, a capping layer CPL may further be arranged on the second electrode EL2 of the light emitting element ED of one or more embodiments. The capping layer CPL may include a multilayer or a single layer.

In one or more embodiments, the capping layer CPL may be an organic layer or an inorganic layer. For example, if (e.g., when) the capping layer CPL contains an inorganic material, the inorganic material may include an alkaline metal compound (e.g., LiF), an alkaline earth metal compound (e.g., MgF₂), SiON, SiNₓ, SiOy, and/or the like.

For example, if (e.g., when) the capping layer CPL includes an organic material, the organic material may include α-NPD, NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol-9-yl)triphenylamine (TCTA), and/or the like, or an epoxy resin, or acrylate such as methacrylate. However, one or more embodiments of the present disclosure is not limited thereto, and the capping layer CPL may include at least one selected from among Compounds P1 to P5:

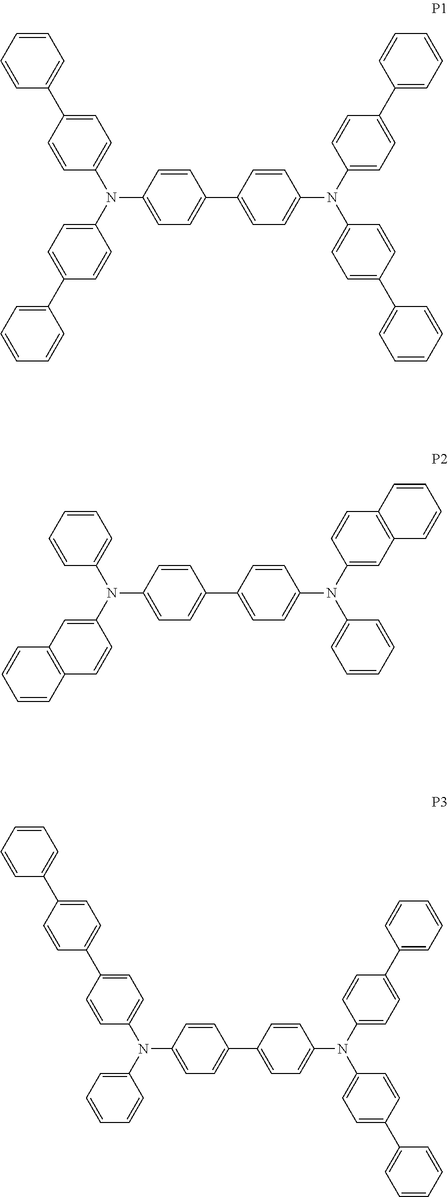 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

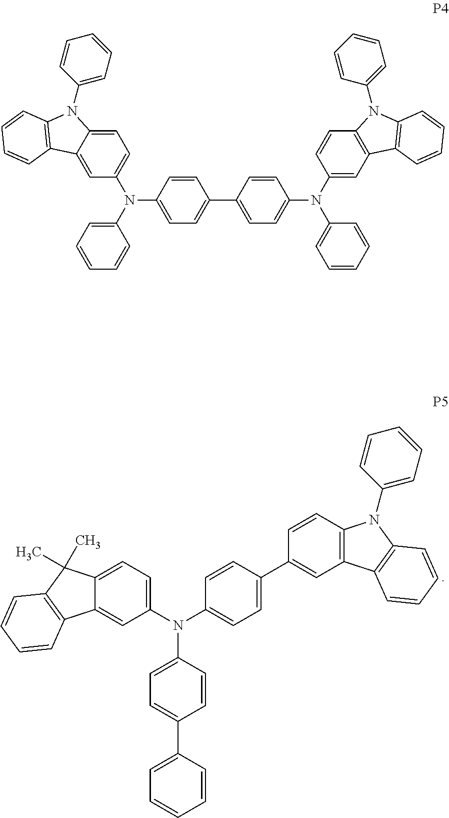 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In one or more embodiments, the refractive index of the capping layer CPL may be about 1.6 or more. For example, with respect to light in a wavelength range of about 550 nm to about 660 nm, the refractive index of the capping layer CPL may be about 1.6 or more.

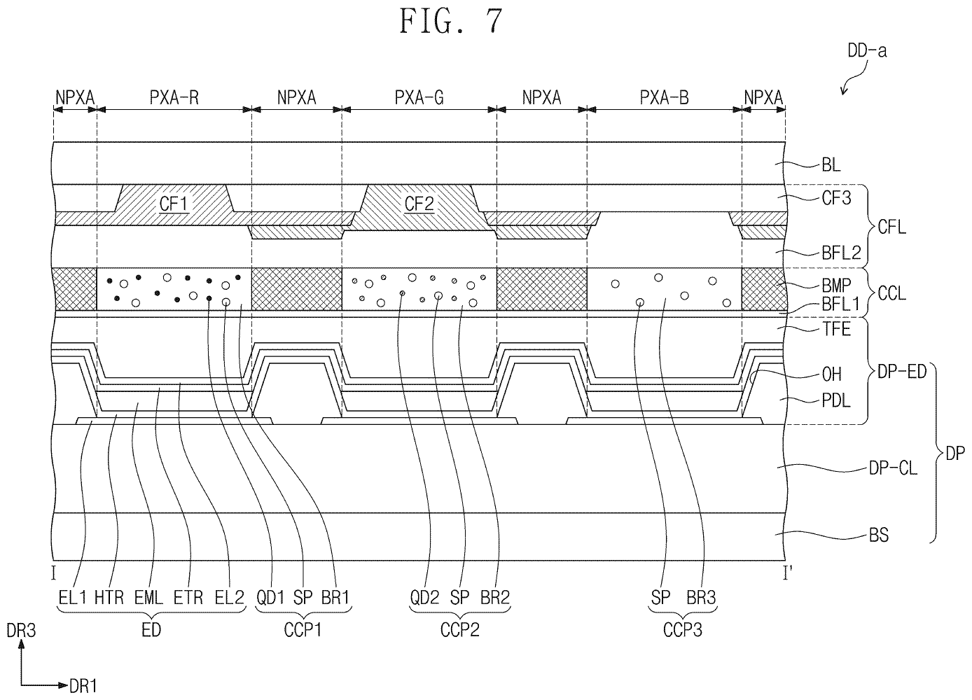

Each of FIGS. 7 to 10 is a cross-sectional view of a display device according to one or more embodiments of the disclosure. Hereinafter, in describing the display devices of embodiments with reference to FIGS. 7 to 10, the duplicated features which have been described in FIGS. 1 to 6 are not described again, but their differences will be mainly described, e.g., in more detail.

Referring to FIG. 7, the display device DD-a according to one or more embodiments may include a display panel DP including a display device layer DP-ED, a light control layer CCL arranged on the display panel DP, and a color filter layer CFL. In one or more embodiments illustrated in FIG. 7, the display panel DP may include a base layer BS, a circuit layer DP-CL provided on the base layer BS, and the display device layer DP-ED, and the display device layer DP-ED may include a light emitting element ED.

The light emitting element ED may include a first electrode EL1, a hole transport region HTR arranged on the first electrode EL1, an emission layer EML arranged on the hole transport region HTR, an electron transport region ETR arranged on the emission layer EML, and a second electrode EL2 arranged on the electron transport region ETR. In one or more embodiments, the structures of the light emitting elements ED of FIGS. 3 to 6 as described herein may be equally applied to the structure of the light emitting element ED illustrated in FIG. 7.

The hole transport region HTR of the light emitting element ED included in the display device DD-a according to one or more embodiments, may include the amine compound of one or more embodiments.

Referring to FIG. 7, the emission layer EML may be arranged in an opening OH defined in a pixel defining film PDL. For example, the emission layer EML which is divided by the pixel defining film PDL and provided corresponding to each light emitting regions PXA-R, PXA-G, and PXA-B may be to emit light in substantially the same wavelength range. In the display device DD-a of one or more embodiments, the emission layer EML may be to emit blue light. In one or more embodiments, unlike the configuration illustrated, in one or more embodiments, the emission layer EML may be provided as a common layer in the entire light emitting regions PXA-R, PXA-G, and PXA-B.

The light control layer CCL may be arranged on the display panel DP. The light control layer CCL may include a light conversion body. The light conversion body may be a quantum dot, a phosphor, and/or the like. The light conversion body may be to emit provided light by converting the wavelength thereof. For example, the light control layer CCL may a layer containing the quantum dot or a layer containing the phosphor.

The light control layer CCL may include a plurality of light control parts CCP1, CCP2 and CCP3. The light control parts CCP1, CCP2, and CCP3 may be spaced and/or apart (e.g., spaced apart or separated) from each other.

Referring to FIG. 7, divided patterns BMP may be arranged between the light control parts CCP1, CCP2 and CCP3 which are spaced and/or apart (e.g., spaced apart or separated) from each other, but one or more embodiments of the present disclosure is not limited thereto. FIG. 7 illustrates that the divided patterns BMP do not overlap the light control parts CCP1, CCP2 and CCP3, but at least a portion of the edges of the light control parts CCP1, CCP2 and CCP3 may overlap the divided patterns BMP.

The light control layer CCL may include a first light control part CCP1 containing a first quantum dot QD1 which converts first color light provided from the light emitting element ED into second color light, a second light control part CCP2 containing a second quantum dot QD2 which converts the first color light into third color light, and a third light control part CCP3 which transmits the first color light.

In one or more embodiments, the first light control part CCP1 may provide red light that is the second color light, and the second light control part CCP2 may provide green light that is the third color light. The third light control part CCP3 may provide blue light by transmitting the blue light that is the first color light provided from the light emitting element ED. For example, the first quantum dot QD1 may be a red quantum dot, and the second quantum dot QD2 may be a green quantum dot. The same as described herein may be applied with respect to the quantum dots QD1 and QD2.

In some embodiments, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include (e.g., may exclude) any quantum dot but include the scatterer SP.

The scatterer SP may be inorganic particles. For example, the scatterer SP may include at least one of TiO₂, ZnO, Al₂O₃, SiO₂, or hollow sphere silica. The scatterer SP may include any one selected from among TiO₂, ZnO, Al₂O₃, SiO₂, and hollow sphere silica, or may be a mixture of at least two materials selected from among TiO₂, ZnO, Al₂O₃, SiO₂, and hollow sphere silica.

The first light control part CCP1, the second light control part CCP2, and the third light control part CCP3 each may include base resins BR1, BR2, and BR3 in which the quantum dots QD1 and QD2 and the scatterer SP are dispersed. In one or more embodiments, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3.

The base resins BR1, BR2, and BR3 are media in which the quantum dots QD1 and QD2 and the scatterer SP are dispersed, and may be formed of one or more suitable resin compositions, which may be generally referred to as a binder. For example, the base resins BR1, BR2, and BR3 may be acrylic-based resins, urethane-based resins, silicone-based resins, epoxy-based resins, and/or the like. The base resins BR1, BR2, and BR3 may be transparent resins. In one or more embodiments, the first base resin BR1, the second base resin BR2, and the third base resin BR3 may be the same as or different from each other.

The light control layer CCL may include a barrier layer BFL1. The barrier layer BFL1 may serve to prevent or reduce the penetration of moisture and/or oxygen (hereinafter, referred to as ‘moisture/oxygen’). The barrier layer BFL1 may block or reduce the light control parts CCP1, CCP2 and CCP3 from being exposed to moisture/oxygen. In one or more embodiments, the barrier layer BFL1 may cover the light control parts CCP1, CCP2, and CCP3. In some embodiments, the barrier layer BFL2 may be provided between the light control parts CCP1, CCP2, and CCP3 and the filters CF1, CF2, and CF3.

The barrier layers BFL1 and BFL2 may include at least one inorganic layer. For example, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, and/or the like. In one or more embodiments, the barrier layers BFL1 and BFL2 may further include an organic film. The barrier layers BFL1 and BFL2 may be formed of a single layer or a plurality of layers.

In the display device DD-a of one or more embodiments, the color filter layer CFL may be arranged on the light control layer CCL. For example, the color filter layer CFL may be directly arranged on the light control layer CCL. In this case, the barrier layer BFL2 may not be provided.

The color filter layer CFL may include filters CF1, CF2, and CF3. The color filter layer CFL may include a first filter CF1 configured to transmit the second color light, a second filter CF2 configured to transmit the third color light, and a third filter CF3 configured to transmit the first color light. For example, the first filter CF1 may be a red filter, the second filter CF2 may be a green filter, and the third filter CF3 may be a blue filter. The filters CF1, CF2, and CF3 each may include a polymeric photosensitive resin and a pigment or dye. The first filter CF1 may include a red pigment or dye, the second filter CF2 may include a green pigment or dye, and the third filter CF3 may include a blue pigment or dye.

In one or more embodiments, one or more embodiments of the present disclosure is not limited thereto, and the third filter CF3 may not include (e.g., may exclude) a pigment or dye. The third filter CF3 may include a polymeric photosensitive resin and may not include (e.g., may exclude) a pigment or dye. The third filter CF3 may be transparent. The third filter CF3 may be formed of a transparent photosensitive resin.

Furthermore, in one or more embodiments, the first filter CF1 and the second filter CF2 may be a yellow filter. The first filter CF1 and the second filter CF2 may not be separated but be provided as one filter.

In one or more embodiments, the color filter layer CFL may further include a light shielding part. The light shielding part may be a black matrix. The light shielding part may include an organic light shielding material or an inorganic light shielding material containing a black pigment or dye. The light shielding part may prevent or reduce light leakage, and may separate boundaries between the adjacent filters CF1, CF2, and CF3.

The first to third filters CF1, CF2, and CF3 may be arranged corresponding to the red light emitting region PXA-R, the green light emitting region PXA-G, and the blue light emitting region PXA-B, respectively.

A base substrate BL may be arranged on the color filter layer CFL. The base substrate BL may be a member which provides a base surface in which the color filter layer CFL, the light control layer CCL, and/or the like are arranged. The base substrate BL may be a glass substrate, a metal substrate, a plastic substrate, and/or the like. However, one or more embodiments of the present disclosure is not limited thereto, and the base substrate BL may be an inorganic layer, an organic layer, or a composite material layer. In some embodiments, unlike the configuration illustrated, in one or more embodiments, the base substrate BL may not be provided.

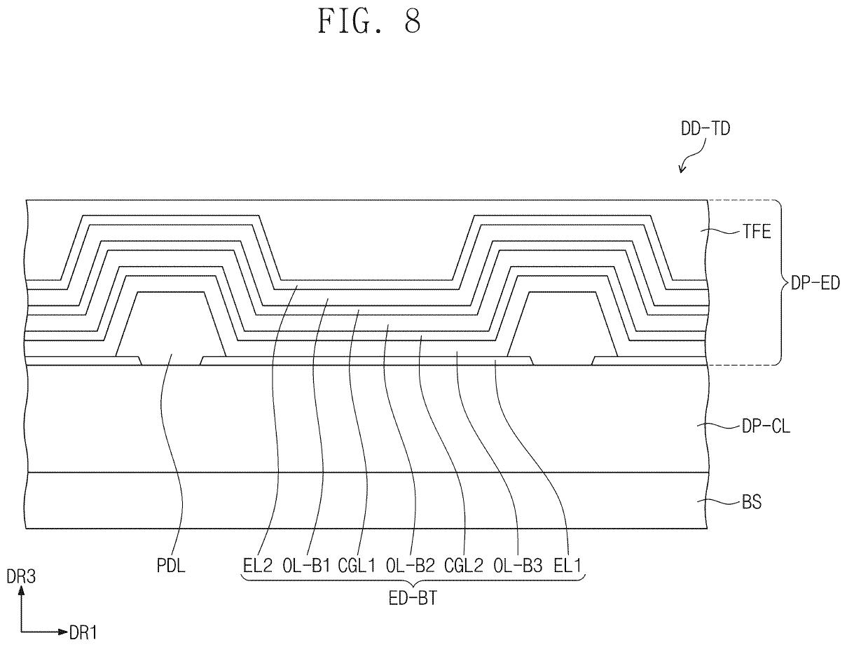

FIG. 8 is a cross-sectional view illustrating a portion of a display device according to one or more embodiments. In the display device DD-TD of one or more embodiments, the light emitting element ED-BT may include a plurality of light emitting structures OL-B1, OL-B2, and OL-B3. The light emitting element ED-BT may include a first electrode EL1 and a second electrode EL2 which face each other, and the plurality of light emitting structures OL-B1, OL-B2, and OL-B3 sequentially stacked in the thickness direction between the first electrode EL1 and the second electrode EL2. The light emitting structures OL-B1, OL-B2, and OL-B3 each may include an emission layer EML (FIG. 7) and a hole transport region HTR (FIG. 7) and an electron transport region ETR (FIG. 7) arranged with the emission layer EML located therebetween.

For example, the light emitting element ED-BT included in the display device DD-TD of one or more embodiments may be a light emitting element having a tandem structure and including a plurality of emission layers EML.

In one or more embodiments illustrated in FIG. 8, all light beams respectively emitted from the light emitting structures OL-B1, OL-B2, and OL-B3 may be blue light. However, one or more embodiments of the present disclosure is not limited thereto, and the light beams respectively emitted from the light emitting structures OL-B1, OL-B2, and OL-B3 may have wavelength ranges different from each other. For example, the light emitting element ED-BT including the plurality of light emitting structures OL-B1, OL-B2, and OL-B3 which emit light beams having wavelength ranges different from each other may be to emit white light.

Charge generation layers CGL1 and CGL2 may be respectively arranged between two of the neighboring light emitting structures OL-B1, OL-B2, and OL-B3. The charge generation layers CGL1 and CGL2 may include a p-type or kind charge generation layer (e.g., p-charge generating layer) and/or an n-type or kind charge generation layer (e.g., n-charge generating layer).

At least one of the light emitting structures OL-B1, OL-B2, and OL-B3 included in the display device DD-TD according to one or more embodiments, may include the amine compound of one or more embodiments.

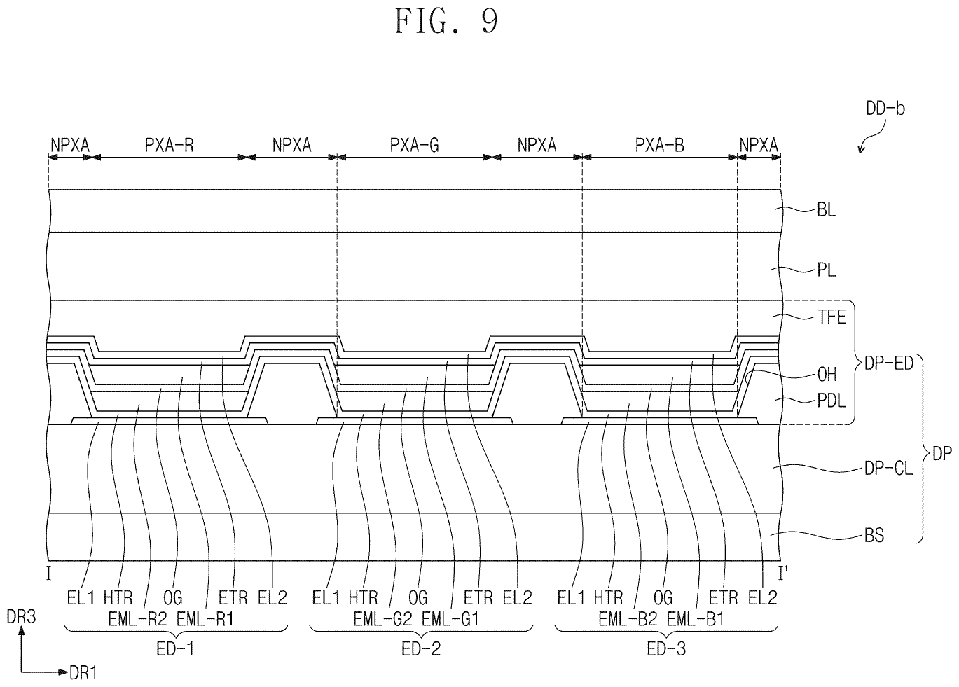

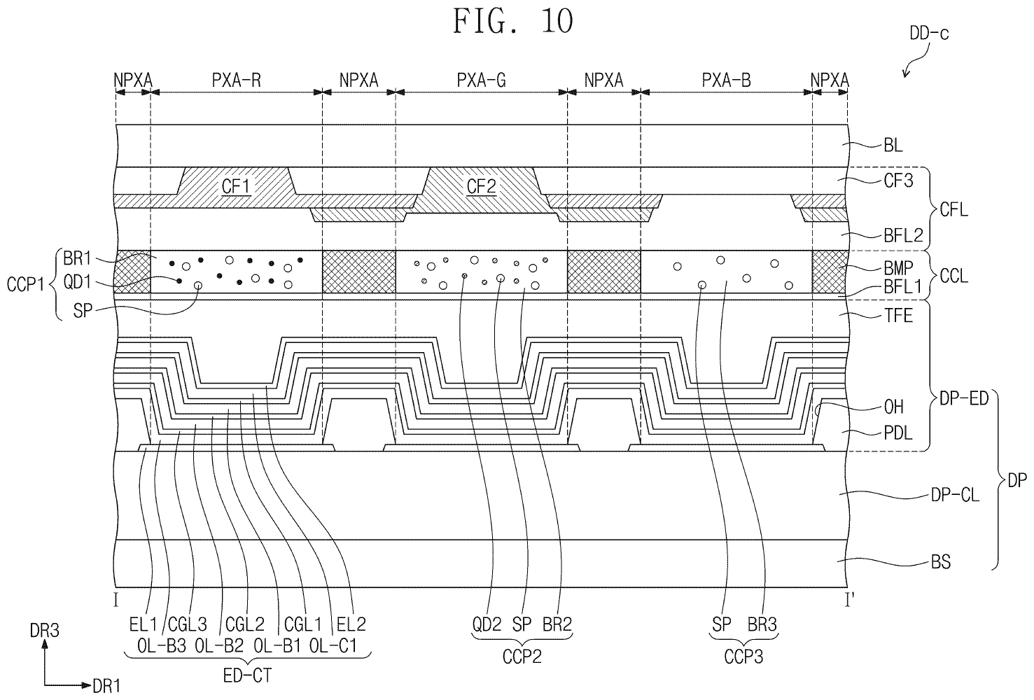

Referring to FIG. 9, the display device DD-b according to one or more embodiments may include light emitting elements ED-1, ED-2, and ED-3 in which two emission layers are stacked. Compared with the display device DD of one or more embodiments illustrated in FIG. 2, one or more embodiments illustrated in FIG. 9 has a difference in that the first to third light emitting elements ED-1, ED-2, and ED-3 each include two emission layers stacked in the thickness direction. In each of the first to third light emitting elements ED-1, ED-2, and ED-3, the two emission layers may be to emit light in substantially the same wavelength region.

The first light emitting element ED-1 may include a first red emission layer EML-R₁ and a second red emission layer EML-R₂. The second light emitting element ED-2 may include a first green emission layer EML-G1 and a second green emission layer EML-G2. In some embodiments, the third light emitting element ED-3 may include a first blue emission layer EML-B1 and a second blue emission layer EML-B2. An emission auxiliary part OG may be arranged between the first red emission layer EML-R₁ and the second red emission layer EML-R₂, between the first green emission layer EML-G1 and the second green emission layer EML-G2, and between the first blue emission layer EML-B1 and the second blue emission layer EML-B2.

The emission auxiliary part OG may include a single layer or a multilayer. The emission auxiliary part OG may include a charge generation layer. For example, the emission auxiliary part OG may include an electron transport region, a charge generation layer, and a hole transport region that are sequentially stacked. The emission auxiliary part OG may be provided as a common layer in the whole of the first to third light emitting elements ED-1, ED-2, and ED-3. However, one or more embodiments of the present disclosure is not limited thereto, and the emission auxiliary part OG may be provided by being patterned within the openings OH defined in the pixel defining film PDL.

The first red emission layer EML-R₁, the first green emission layer EML-G1, and the first blue emission layer EML-B1 may be arranged between the electron transport region ETR and the emission auxiliary part OG. The second red emission layer EML-R₂, the second green emission layer EML-G2, and the second blue emission layer EML-B2 may be arranged between the emission auxiliary part OG and the hole transport region HTR.

For example, the first light emitting element ED-1 may include the first electrode EL1, the hole transport region HTR, the second red emission layer EML-R₂, the emission auxiliary part OG, the first red emission layer EML-R₁, the electron transport region ETR, and the second electrode EL2 that are sequentially stacked. The second light emitting element ED-2 may include the first electrode EL1, the hole transport region HTR, the second green emission layer EML-G2, the emission auxiliary part OG, the first green emission layer EML-G1, the electron transport region ETR, and the second electrode EL2 that are sequentially stacked. The third light emitting element ED-3 may include the first electrode EL1, the hole transport region HTR, the second blue emission layer EML-B2, the emission auxiliary part OG, the first blue emission layer EML-B1, the electron transport region ETR, and the second electrode EL2 that are sequentially stacked.

In one or more embodiments, an optical auxiliary layer PL may be arranged on the display device layer DP-ED. The optical auxiliary layer PL may include a polarizing layer. The optical auxiliary layer PL may be arranged on the display panel DP and control reflected light in the display panel DP due to external light. Unlike the configuration illustrated, the optical auxiliary layer PL in the display device according to one or more embodiments may not be provided.

Unlike FIGS. 8 and 9, FIG. 10 illustrates that a display device DD-c includes four light emitting structures OL-B1, OL-B2, OL-B3, and OL-C1. A light emitting element ED-CT may include a first electrode EL1 and a second electrode EL2 which face each other, and first to fourth light emitting structures OL-B1, OL-B2, OL-B3, and OL-C1 that are sequentially stacked in the thickness direction between the first electrode EL1 and the second electrode EL2. Light emitting structures OL-C1, OL-B1, OL-B2, and OL-B3 are stacked successively, and the charge generation layer CGL1 is disposed between light emitting structures OL-B1 and OL-C1, the charge generation layer CGL2 is disposed between light emitting structures OL-B1 and OL-B2, and the charge generation layer CGL3 is disposed between light emitting structures OL-B2 and OL-B3. Among the four light emitting structures, the first to third light emitting structures OL-B1, OL-B2, and OL-B3 may be to emit blue light, and the fourth light emitting structure OL-C1 may be to emit green light. However, one or more embodiments of the disclosure is not limited thereto, and the first to fourth light emitting structures OL-B1, OL-B2, OL-B3, and OL-C1 may be to emit light beams in different wavelength regions.

The charge generation layers CGL1, CGL2, and CGL3 arranged between adjacent light emitting structures OL-B1, OL-B2, OL-B3, and OL-C1 may include a p-type or kind charge generation layer (e.g., p-charge generating layer) and/or an n-type or kind charge generation layer (e.g., n-charge generating layer).

In at least one selected from among the light emitting structures OL-B1, OL-B2, OL-B3 and OL-C1, included in the display device DD-c of one or more embodiments, the amine compound of one or more embodiments may be included.

The light emitting element ED according to one or more embodiments of the disclosure may include the amine compound of one or more embodiments in at least one functional layer arranged between the first electrode EL1 and the second electrode EL2 to show improved emission efficiency and improved lifetime characteristics. The light emitting element ED according to one or more embodiments may include the amine compound of one or more embodiments in at least one selected from among a hole transport region HTR, an emission layer EML, and an electron transport region ETR, arranged between the first electrode EL1 and the second electrode EL2, or in a capping layer CPL. For example, the amine compound according to one or more embodiments may be included in the hole transport region HTR of the light emitting element ED of one or more embodiments, and the light emitting element ED of one or more embodiments may show high efficiency and long lifetime characteristics.

The amine compound of one or more embodiments includes a first core, and second and third substituents and may improve the stability of a material and improve hole transport properties. Accordingly, the efficiency and lifetime of the light emitting element including the amine compound of one or more embodiments may be improved. For example, the light emitting element of one or more embodiments may include the amine compound according to one or more embodiments in an electron blocking layer to show improved efficiency and lifetime characteristics.

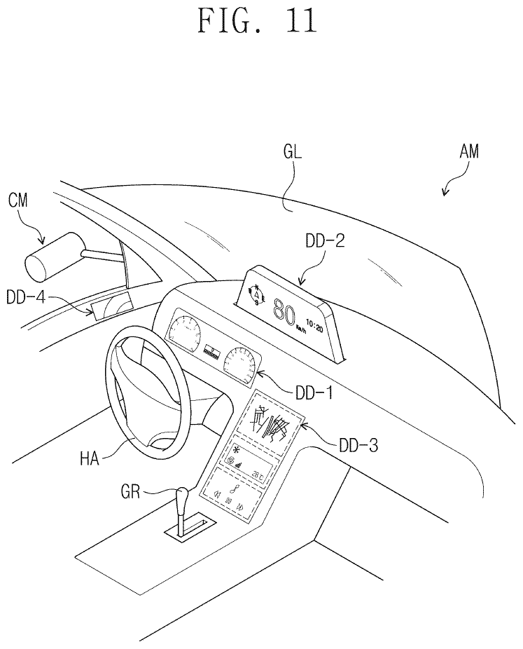

FIG. 11 is a view illustrating a vehicle AM in which first to fourth display devices DD-1, DD-2, DD-3, and DD-4 are arranged. At least one selected from among the first to fourth display devices DD-1, DD-2, DD-3, and DD-4 may include the same configuration as the display devices DD, DD-TD, DD-a, DD-b, and DD-c as described with reference to FIGS. 1, 2, and 7 to 10.

FIG. 11 illustrates a vehicle AM, but this is an example, and the first to fourth display devices DD-1, DD-2, DD-3, and DD-4 may be arranged in another transportation modes such as bicycles, motorcycles, trains, ships, and airplanes. In some embodiments, at least one selected from among the first to fourth display devices DD-1, DD-2, DD-3, and DD-4 including the same configuration as the display devices DD, DD-TD, DD-a, DD-b, and DD-c of one or more embodiments may be employed in a personal computer, a laptop computer, a personal digital terminal, a game console, a portable electronic device, a television, a monitor, an outdoor billboard, and/or the like. In some embodiments, these are merely provided as embodiments, and thus may be employed in other electronic apparatuses unless departing from the present disclosure.

At least one selected from among the first to fourth display devices DD-1, DD-2, DD-3, and DD-4 may include the light emitting element ED of one or more embodiments as described with reference to FIGS. 3 to 6.

At least one selected from among the first to fourth display devices DD-1, DD-2, DD-3, and DD-4 includes the light emitting element ED of one or more embodiments described with reference to FIGS. 3 to 6. can do. The light emitting element ED of one or more embodiments may include the amine compound of one or more embodiments. At least one selected from among the first to fourth display devices DD-1, DD-2, DD-3, and DD-4 includes a light emitting element ED including an amine compound according to one or more embodiments, and the display lifetime is improved.

Referring to FIG. 11, the vehicle AM may include a steering wheel HA and a gear GR for driving the vehicle AM. In some embodiments, the vehicle AM may include a front window GL arranged so as to face the driver.

The first display device DD-1 may be arranged in a first region overlapping the steering wheel HA. For example, the first display device DD-1 may be a digital cluster which displays first information of the vehicle AM. The first information may include a first scale which indicates a driving speed of the vehicle AM, a second scale which indicates an engine speed (that is, revolutions per minute (RPM)), an image which indicates a fuel state, and/or the like. A first scale and a second scale may be indicated as a digital image.

The second display device DD-2 may be arranged in a second region facing the driver's seat and overlapping the front window GL. The driver's seat may be a seat in which the steering wheel HA is arranged. For example, the second display device DD-2 may be a head up display (HUD) which displays second information of the vehicle AM. The second display device DD-2 may be optically transparent. The second information may include digital numbers which indicate a driving speed, and may further include information such as the current time. Unlike the configuration illustrated, the second information of the second display device DD-2 may be projected to the front window GL to be displayed.

The third display device DD-3 may be arranged in a third region adjacent to the gear GR. For example, the third display device DD-3 may be arranged between the driver's seat and the passenger seat and may be a center information display (CID) for a vehicle AM for displaying third information. The passenger seat may be a seat spaced and/or apart (e.g., spaced apart or separated) from the driver's seat with the gear GR arranged therebetween. The third information may include information about traffic (e.g., navigation information), playing music or radio or a video (or an image), temperatures inside the vehicle AM, and/or the like.

The fourth display device DD-4 may be spaced and/or apart (e.g., spaced apart or separated) from the steering wheel HA and the gear GR, and may be arranged in a fourth region adjacent to the side of the vehicle AM. For example, the fourth display device DD-4 may be a digital side-view mirror which displays fourth information. The fourth display device DD-4 may display an image outside the vehicle AM taken by a camera module CM arranged outside the vehicle AM. The fourth information may include an image outside the vehicle AM.

The herein-described first to fourth information may be examples, and the first to fourth display devices DD-1, DD-2, DD-3, and DD-4 may further display information about the inside and outside of the vehicle AM. The first to fourth information may include different information. However, one or more embodiments of the disclosure is not limited thereto, and a part of the first to fourth information may include the same information as one another.

Hereinafter, referring to one or more embodiments and comparative embodiments, the amine compound according to one or more embodiments and the light emitting element according to one or more embodiments of the present disclosure will be explained in particular. In some embodiments, one or more embodiments are illustrations to assist the understanding of the present disclosure, but the scope of the present disclosure is not limited thereto.

### Examples

### 1. Synthesis of Amine Compounds

First, the synthetic methods of the amine compounds according to one or more embodiments will be explained in particular illustrating the synthetic methods of Compound 209, 295, 296, 300, 327, 480, 495, 504, 566, 584, 784, 923, and 968. In some embodiments, the synthetic methods of the amine compounds explained hereinafter are merely example embodiments, and the synthetic method of the amine compound according to one or more embodiments of the present disclosure is not limited to one or more embodiments as described herein.

### (1) Synthesis of Intermediate Compound IM-1 to Intermediate Compound IM-15

### 1) Synthesis of Intermediate Compound IM-1

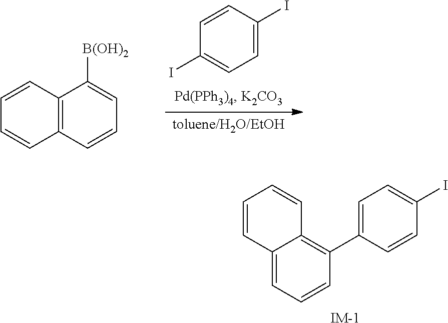

Under an Ar₁ atmosphere, to a 1 L, three-neck flask, naphthalen-1-ylboronic acid (20.0 g), 1,4-diiodobenzene (38.3 g), tetrakis(triphenylphosphine)palladium(0) (Pd(PPh₃)₄, 6.7 g), and potassium carbonate (K₂CO₃, 32.1 g) were added and dissolved in a mixture solvent of toluene, water and ethanol (10:2:1, 500 mL), followed by heating and stirring at about 80° C. for about 4 hours. Then, water was added, extraction was performed with CH₂Cl₂, organic layers were collected and dried over MgSO₄, and the solvent was removed by distillation under a reduced pressure. The crude product thus obtained was separated by silica gel column chromatography and subjected to recrystallization to obtain 22.7 g (yield 58%) of Intermediate Compound IM-1. The molecular weight of Intermediate Compound IM-1, measured by FAB-MS was 330.

### 2) Synthesis of Intermediate Compound IM-2

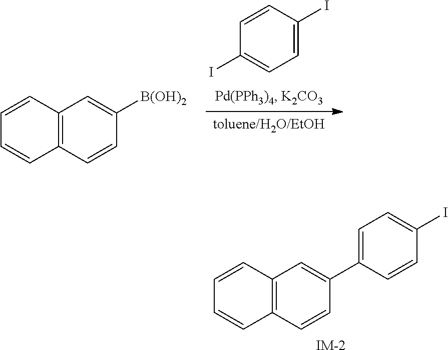

According to substantially the same method as the synthesis of Intermediate Compound IM-1, 23.8 g (yield 62%) of Intermediate Compound IM-2 was obtained from naphthalen-2-ylboronic acid (20.0 g) and 1,4-diiodobenzene (38.3 g). The molecular weight of Intermediate Compound IM-2, measured by FAB-MS was 330.

### 3) Synthesis of Intermediate Compound IM-3

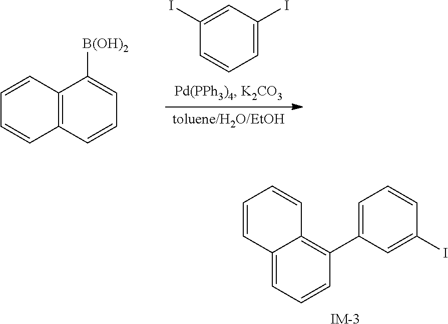

According to substantially the same method as the synthesis of Intermediate Compound IM-1, 23.0 g (yield 60%) of Intermediate Compound IM-3 was obtained from naphthalen-1-ylboronic acid (20.0 g) and 1,3-diiodobenzene (38.3 g). The molecular weight of Intermediate Compound IM-3, measured by FAB-MS was 330.

### 4) Synthesis of Intermediate Compound IM-4

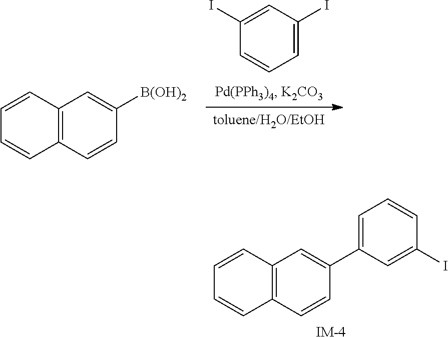

According to substantially the same method as the synthesis of Intermediate Compound IM-1, 21.1 g (yield 55%) of Intermediate Compound IM-4 was obtained from naphthalen-2-ylboronic acid (20.0 g) and 1,3-diiodobenzene (38.3 g). The molecular weight of Intermediate Compound IM-4, measured by FAB-MS was 330.

### 5) Synthesis of Intermediate Compound IM-5

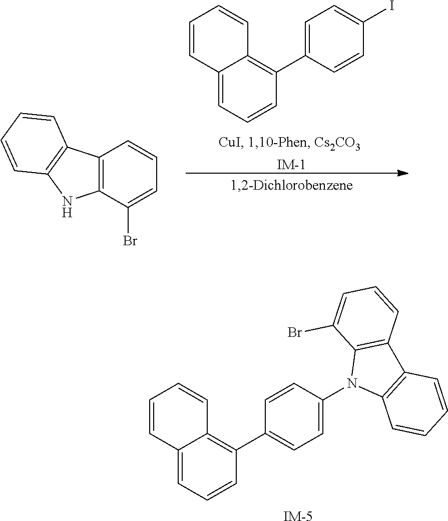

Under an Ar₁ atmosphere, to a 1 L, three-neck flask, 1-bromocarbazole (20.0 g), Intermediate Compound IM-1 (26.8 g), copper(I) iodide (CuI, 1.5 g), 1,10-phenanthroline (1,10-Phen, 2.9 g), and cesium carbonate (Cs₂CO₃, 52 g) were added and dissolved in 1,2-dichlorobenzene (400 mL), followed by heating and stirring at about 150° C. for about 12 hours. Then, water was added, extraction was performed with CH₂Cl₂, organic layers were collected and dried over MgSO₄, and the solvent was removed by distillation under a reduced pressure. The crude product thus obtained was separated by silica gel column chromatography and subjected to recrystallization to obtain 20.0 g (yield 55%) of Intermediate Compound IM-5. The molecular weight of Intermediate Compound IM-5, measured by FAB-MS was 448.

### 6) Synthesis of Intermediate Compound IM-6

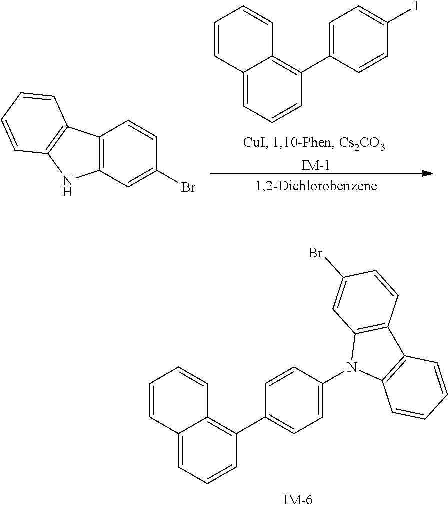

According to substantially the same method as the synthesis of Intermediate Compound IM-5, 21.1 g (yield 58%) of Intermediate Compound IM-6 was obtained from 2-bromocarbazole (20.0 g) and Intermediate Compound IM-1 (26.8 g). The molecular weight of Intermediate Compound IM-6, measured by FAB-MS was 448.

### 7) Synthesis of Intermediate Compound IM-7

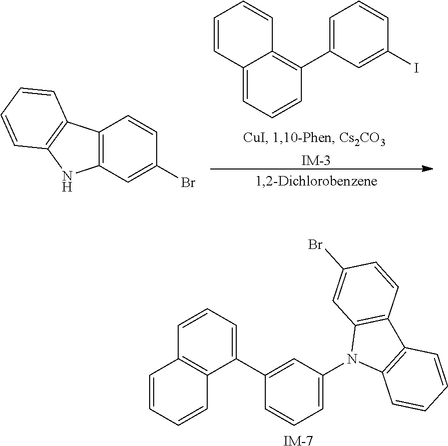

According to substantially the same method as the synthesis of Intermediate Compound IM-5, 18.9 g (yield 52%) of Intermediate Compound IM-7 was obtained from 2-bromocarbazole (20.0 g) and Intermediate Compound IM-3 (26.8 g). The molecular weight of Intermediate Compound IM-7, measured by FAB-MS was 448.

### 8) Synthesis of Intermediate Compound IM-8

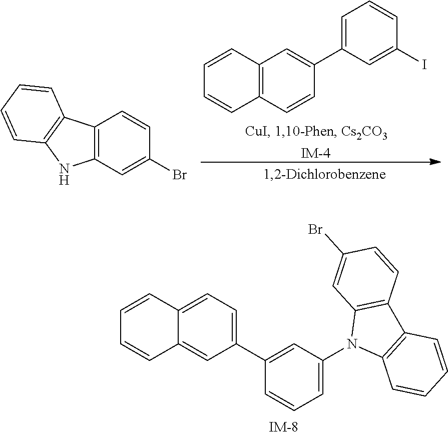

According to substantially the same method as the synthesis of Intermediate Compound IM-5, 19.6 g (yield 54%) of Intermediate Compound IM-8 was obtained from 2-bromocarbazole (20.0 g) and Intermediate Compound IM-4 (26.8 g). The molecular weight of Intermediate Compound IM-8, measured by FAB-MS was 448.

### 9) Synthesis of Intermediate Compound IM-9 to Intermediate Compound IM-11

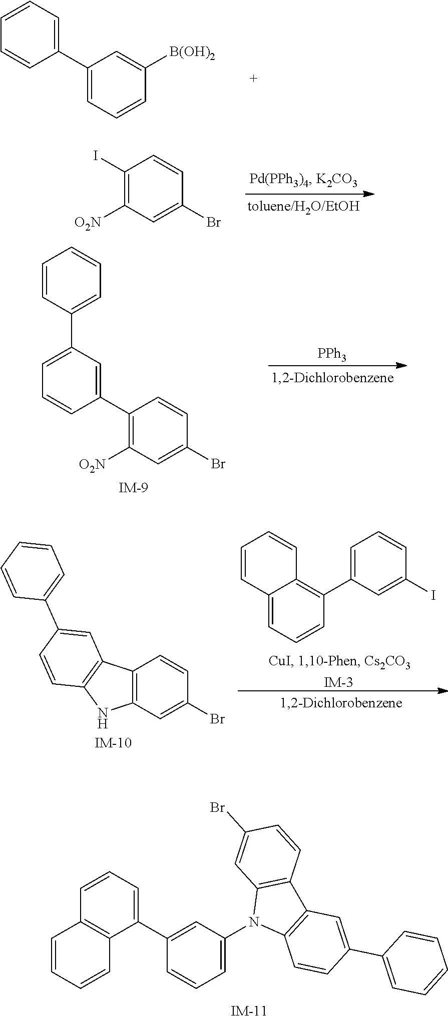

Under an Ar₁ atmosphere, to a 1 L, three-neck flask, [1,1′-biphenyl]-3-ylboronic acid (10.0 g), 4-bromo-1-iodo-2-nitrobenzene (16.5 g), Pd(PPh₃)₄ (2.9 g), and K₂CO₃ (13.9 g) were added and dissolved in a mixture solvent of toluene, water and ethanol (10:2:1, 300 mL), followed by heating and stirring at about 80° C. for about 4 hours. Then, water was added, extraction was performed with CH₂Cl₂, organic layers were collected and dried over MgSO₄, and the solvent was removed by distillation under a reduced pressure. The crude product thus obtained was separated by silica gel column chromatography and subjected to recrystallization to obtain 13.6 g (yield 76%) of Intermediate Compound IM-9. The molecular weight of Intermediate Compound IM-9, measured by FAB-MS was 354.

Under an Ar₁ atmosphere, to a 500 mL, three-neck flask, Intermediate Compound IM-9 (10.0 g), and triphenylphosphine (PPh₃, 22.2 g) were added and dissolved in 1,2-dichlorobenzene (200 mL), followed by heating and stirring at about 180° C. for about 12 hours. Then, water was added, extraction was performed with CH₂Cl₂, organic layers were collected and dried over MgSO₄, and the solvent was removed by distillation under a reduced pressure. The crude product thus obtained was separated by silica gel column chromatography and subjected to recrystallization to obtain 7.7 g (yield 85%) of Intermediate Compound IM-10. The molecular weight of Intermediate Compound IM-10, measured by FAB-MS was 322.

According to substantially the same method as the synthesis of Intermediate Compound IM-5, 4.5 g (yield 56%) of Intermediate Compound IM-11 was obtained from Intermediate Compound IM-10 (5.0 g) and Intermediate Compound IM-3 (5.1 g). The molecular weight of Intermediate Compound IM-11, measured by FAB-MS was 524.

### 10) Synthesis of Intermediate Compound IM-12

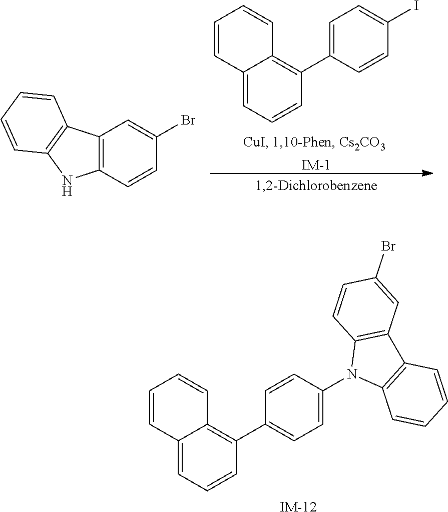

According to substantially the same method as the synthesis of Intermediate Compound IM-5, 16.4 g (yield 45%) of Intermediate Compound IM-12 was obtained from 3-bromocarbazole (20.0 g) and Intermediate Compound IM-1 (26.8 g). The molecular weight of Intermediate Compound IM-12, measured by FAB-MS was 448.

### 11) Synthesis of Intermediate Compound IM-13

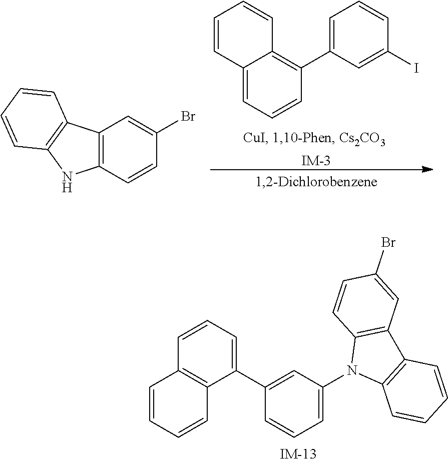

According to substantially the same method as the synthesis of Intermediate Compound IM-5, 15.3 g (yield 42%) of Intermediate Compound IM-13 was obtained from 3-bromocarbazole (20.0 g) and Intermediate Compound IM-3 (26.8 g). The molecular weight of Intermediate Compound IM-13, measured by FAB-MS was 448.

### 12) Synthesis of Intermediate Compound IM-14

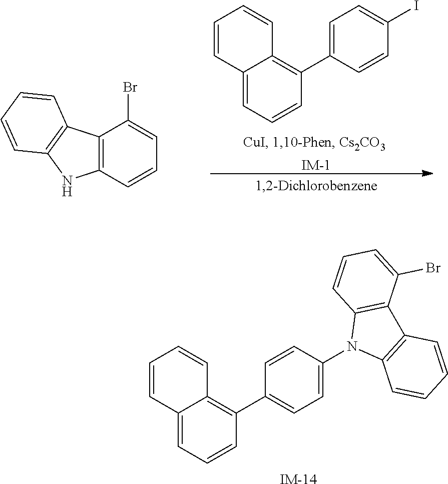

According to substantially the same method as the synthesis of Intermediate Compound IM-5, 19.6 g (yield 54%) of Intermediate Compound IM-14 was obtained from 4-bromocarbazole (20.0 g) and Intermediate Compound IM-1 (26.8 g). The molecular weight of Intermediate Compound IM-14, measured by FAB-MS was 448.

### 13) Synthesis of Intermediate Compound IM-15

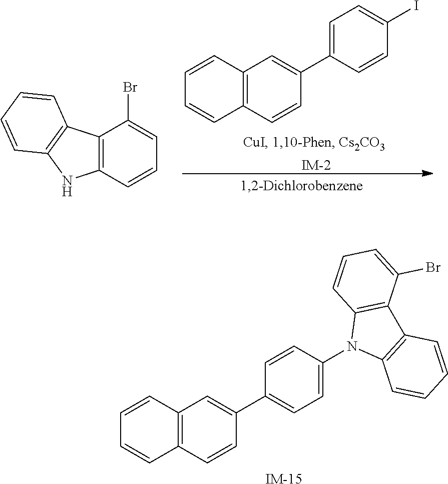

According to substantially the same method as the synthesis of Intermediate Compound IM-5, 19.6 g (yield 54%) of Intermediate Compound IM-15 was obtained from 4-bromocarbazole (20.0 g) and Intermediate Compound IM-2 (26.8 g). The molecular weight of Intermediate Compound IM-15, measured by FAB-MS was 448.

### (2) Synthesis of Amine Compound 209

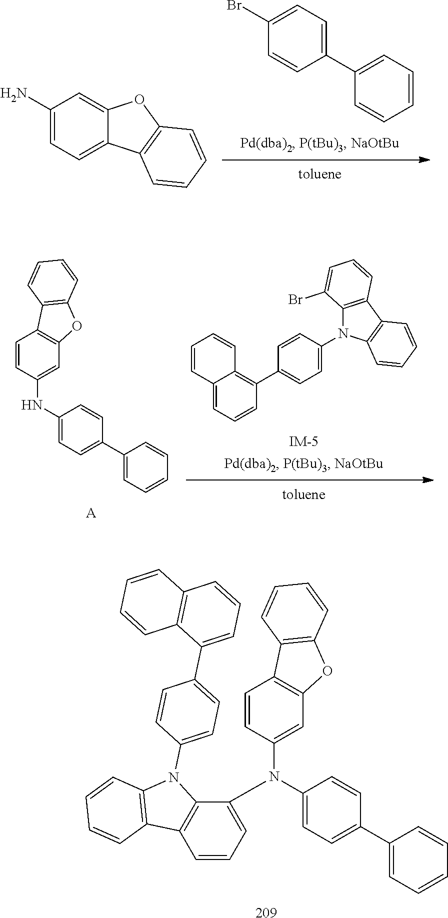

### 1) Synthesis of Intermediate Compound A

Under an Ar₁ atmosphere, to a 1 L, three-neck flask, dibenzo[b,d]furan-3-amine (20.0 g), 4-bromo-1,1′-biphenyl (25.4 g), bis(dibenzylideneacetone)palladium(0) (Pd(dba)₂, 3.1 g), and sodium tert-butoxide (NaOtBu, 15.7 g) were added and dissolved in toluene (400 mL), and tri-tert-butylphosphine (P(tBu)₃, 2.0 M in toluene, 5.5 mL) was added thereto, followed by stirring at room temperature for about 4 hours. Then, water was added, extraction was performed with CH₂Cl₂, organic layers were collected and dried over MgSO₄, and the solvent was removed by distillation under a reduced pressure. The crude product thus obtained was separated by silica gel column chromatography and subjected to recrystallization to obtain 30.7 g (yield 84%) of Intermediate Compound A. The molecular weight of Intermediate Compound A, measured by FAB-MS was 335.

### 2) Synthesis of Amine Compound 209

Under an Ar₁ atmosphere, to a 300 mL, three-neck flask, Intermediate Compound A (5.0 g), Intermediate Compound IM-5 (6.6 g), Pd(dba)₂ (0.4 g), and NaOtBu (2.1 g) were added and dissolved in toluene (100 mL), and P(tBu)₃ (2.0 M in toluene, 0.7 mL) was added thereto, followed by heating and refluxing for about 24 hours. Then, water was added, extraction was performed with CH₂Cl₂, organic layers were collected and dried over MgSO₄, and the solvent was removed by distillation under a reduced pressure. The crude product thus obtained was separated by silica gel column chromatography to obtain 3.7 g (yield 36%) of Amine Compound 209. The molecular weight of Amine Compound 209, measured by FAB-MS was 702.

### (3) Synthesis of Amine Compound 295

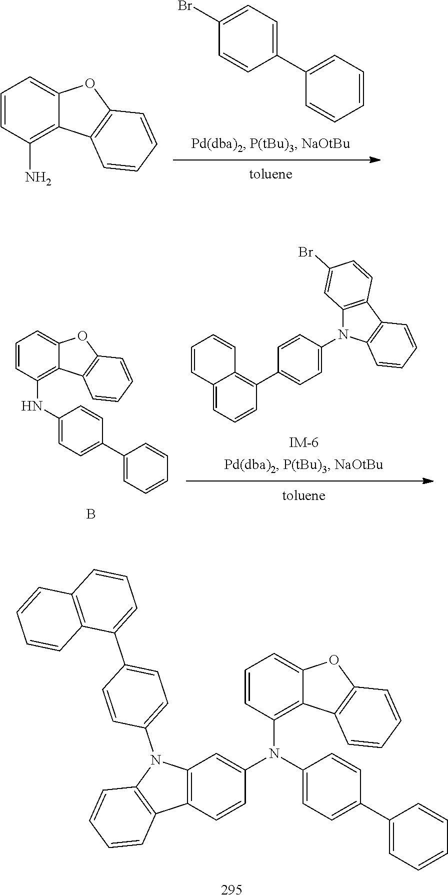

According to substantially the same method as the synthesis of Intermediate Compound A, 27.4 g (yield 75%) of Intermediate Compound B was obtained from dibenzo[b,d]furan-1-amine (20.0 g) and 4-bromo-1,1′-biphenyl (25.4 g). The molecular weight of Intermediate Compound B, measured by FAB-MS was 335.

According to substantially the same method as the synthesis of Amine Compound 209, 7.8 g (yield 75%) of Amine Compound 295 was obtained from Intermediate Compound B (5.0 g) and Intermediate Compound IM-6 (6.6 g). The molecular weight of Amine Compound 295, measured by FAB-MS was 702.

### (4) Synthesis of Amine Compound 296

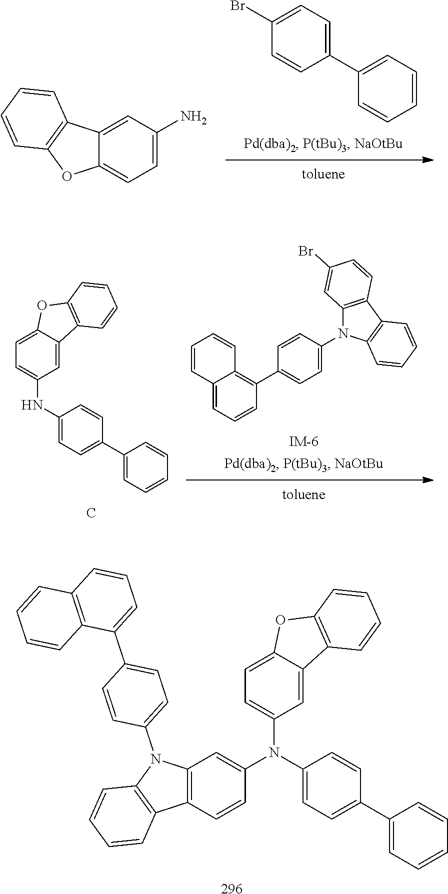

According to substantially the same method as the synthesis of Intermediate Compound A, 20.1 g (yield 55%) of Intermediate Compound C was obtained from dibenzo[b,d]furan-2-amine (20.0 g) and 4-bromo-1,1′-biphenyl (25.4 g). The molecular weight of Intermediate Compound C, measured by FAB-MS was 335.

According to substantially the same method as the synthesis of Amine Compound 209, 7.3 g (yield 70%) of Amine Compound 296 was obtained from Intermediate Compound C (5.0 g) and Intermediate Compound IM-6 (6.6 g). The molecular weight of Amine Compound 296, measured by FAB-MS was 702.

### (5) Synthesis of Amine Compound 300

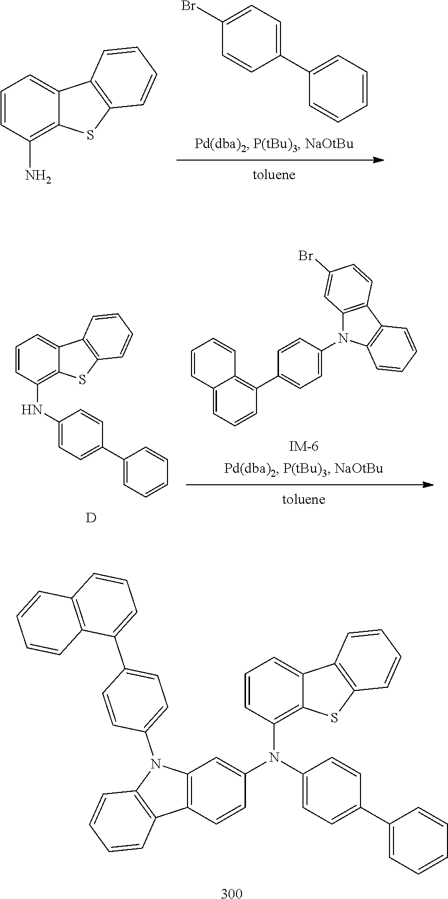

According to substantially the same method as the synthesis of Intermediate Compound A, 20.8 g (yield 59%) of Intermediate Compound D was obtained from dibenzo[b,d]thiophen-4-amine (20.0 g) and 4-bromo-1,1′-biphenyl (23.4 g). The molecular weight of Intermediate Compound D, measured by FAB-MS was 351.

According to substantially the same method as the synthesis of Amine Compound 209, 7.1 g (yield 70%) of Amine Compound 300 was obtained from Intermediate Compound D (5.0 g) and Intermediate Compound IM-6 (6.6 g). The molecular weight of Amine Compound 300, measured by FAB-MS was 718.

### (6) Synthesis of Amine Compound 327

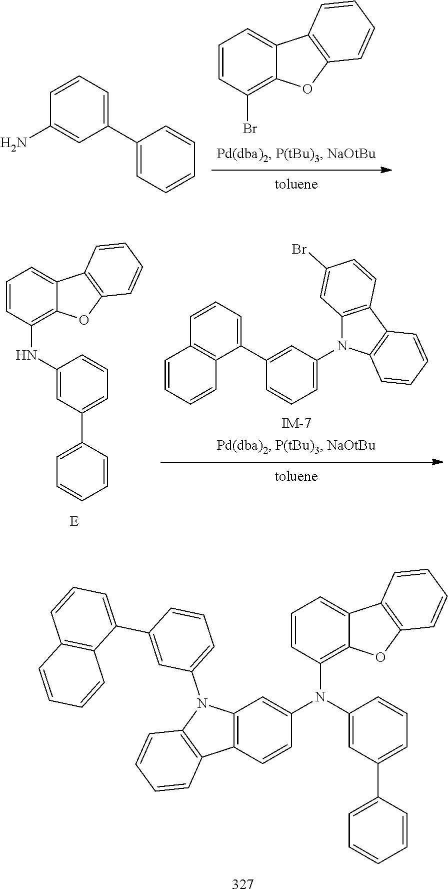

According to substantially the same method as the synthesis of Intermediate Compound A, 25.7 g (yield 65%) of Intermediate Compound E was obtained from [1,1′-biphenyl]-3-amine (20.0 g) and 4-bromodibenzo[b,d]furan (29.2 g). The molecular weight of Intermediate Compound E, measured by FAB-MS was 335.

According to substantially the same method as the synthesis of Amine Compound 209, 7.9 g (yield 76%) of Amine Compound 327 was obtained from Intermediate Compound E (5.0 g) and Intermediate Compound IM-7 (6.7 g). The molecular weight of Amine Compound 327, measured by FAB-MS was 702.

### (7) Synthesis of Amine Compound 480

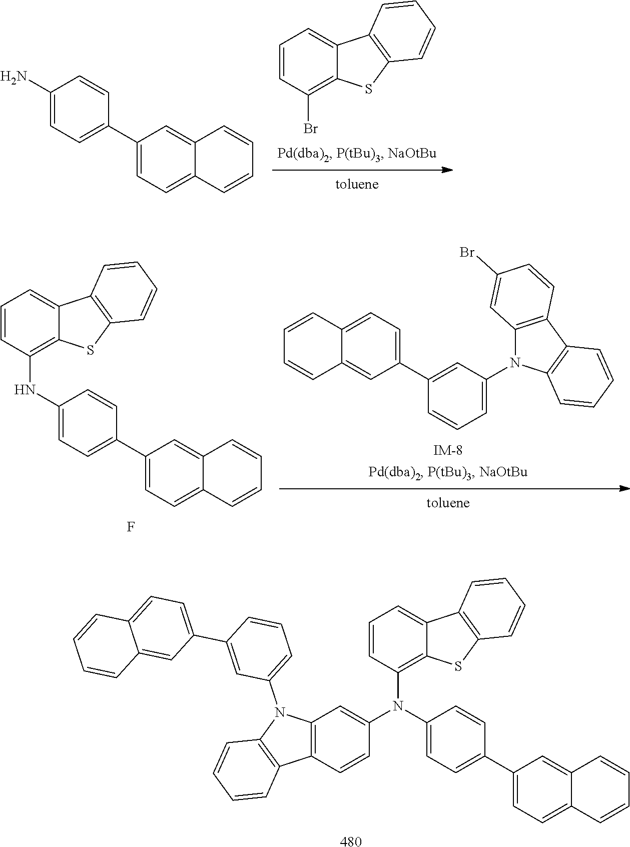

According to substantially the same method as the synthesis of Intermediate Compound A, 29.6 g (yield 81%) of Intermediate Compound F was obtained from 4-(naphthalen-2-yl)aniline (20.0 g) and 4-bromodibenzo[b,d]thiophene (29.2 g). The molecular weight of Intermediate Compound F, measured by FAB-MS was 401.

According to substantially the same method as the synthesis of Amine Compound 209, 6.8 g (yield 71%) of Amine Compound 480 was obtained from Intermediate Compound F (5.0 g) and Intermediate Compound IM-8 (5.6 g). The molecular weight of Amine Compound 480, measured by FAB-MS was 768.

### (8) Synthesis of Amine Compound 495

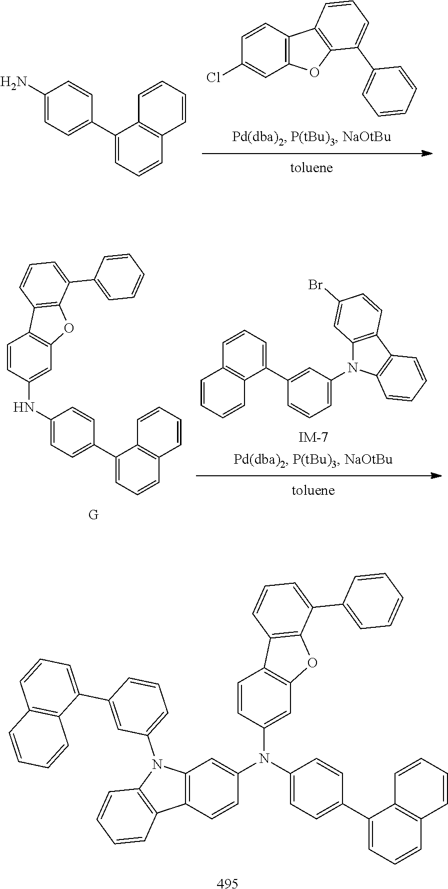

According to substantially the same method as the synthesis of Intermediate Compound A, 21.4 g (yield 51%) of Intermediate Compound G was obtained from 4-(naphthalen-2-yl)aniline (20.0 g) and 3-chloro-6-phenyldibenzo[b,d]furan (25.4 g). The molecular weight of Intermediate Compound G, measured by FAB-MS was 461.

According to substantially the same method as the synthesis of Amine Compound 209, 5.8 g (yield 65%) of Amine Compound 495 was obtained from Intermediate Compound G (5.0 g) and Intermediate Compound IM-7 (4.9 g). The molecular weight of Amine Compound 495, measured by FAB-MS was 829.

### (9) Synthesis of Amine Compound 504

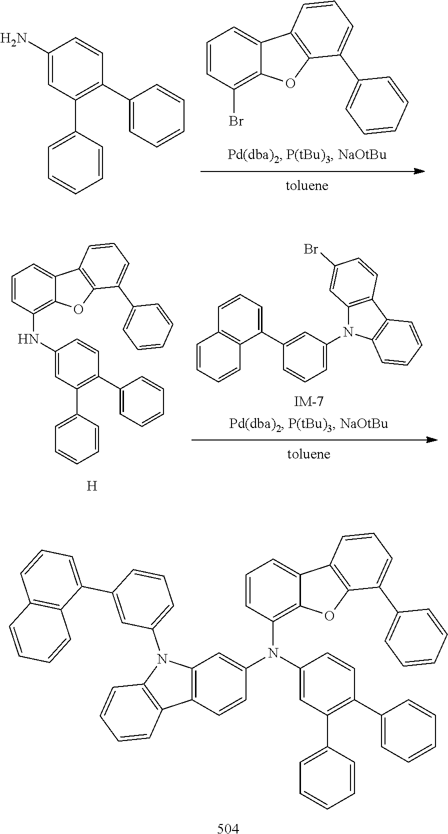

According to substantially the same method as the synthesis of Intermediate Compound A, 34.5 g (yield 87%) of Intermediate Compound H was obtained from [1,1′:2′,1″-terphenyl]-4′-amine (20.0 g) and 4-bromo-6-phenyldibenzo[b,d]furan (26.3 g). The molecular weight of Intermediate Compound H, measured by FAB-MS was 487.

According to substantially the same method as the synthesis of Amine Compound 209, 5.7 g (yield 65%) of Amine Compound 504 was obtained from Intermediate Compound H (5.0 g) and Intermediate Compound IM-7 (4.6 g). The molecular weight of Amine Compound 504, measured by FAB-MS was 855.

### (10) Synthesis of Amine Compound 566

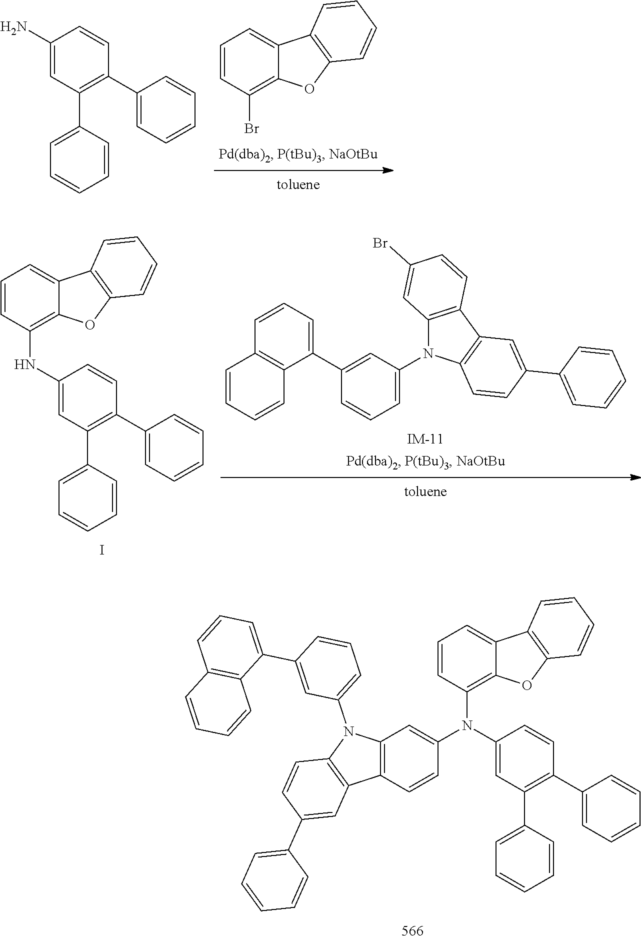

According to substantially the same method as the synthesis of Intermediate Compound A, 28.1 g (yield 84%) of Intermediate Compound I was obtained from [1,1′:2′,1″-terphenyl]-4′-amine (20.0 g) and 4-bromodibenzo[b,d]furan (20.1 g). The molecular weight of Intermediate Compound I, measured by FAB-MS was 411.

According to substantially the same method as the synthesis of Amine Compound 209, 6.3 g (yield 61%) of Amine Compound 566 was obtained from Intermediate Compound I (5.0 g) and Intermediate Compound IM-11 (5.4 g). The molecular weight of Amine Compound 566, measured by FAB-MS was 855.

### (11) Synthesis of Amine Compound 584

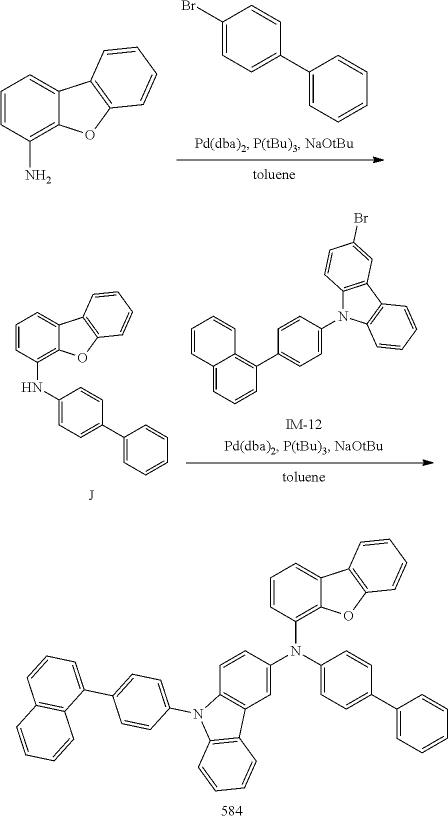

According to substantially the same method as the synthesis of Intermediate Compound A, 29.2 g (yield 80%) of Intermediate Compound J was obtained from dibenzo[b,d]furan-4-amine (20.0 g) and 4-bromo-1,1′-biphenyl (25.4 g). The molecular weight of Intermediate Compound J, measured by FAB-MS was 335.

According to substantially the same method as the synthesis of Amine Compound 209, 7.9 g (yield 76%) of Amine Compound 584 was obtained from Intermediate Compound J (5.0 g) and Intermediate Compound IM-12 (6.7 g). The molecular weight of Amine Compound 584, measured by FAB-MS was 702.

### (12) Synthesis of Amine Compound 784

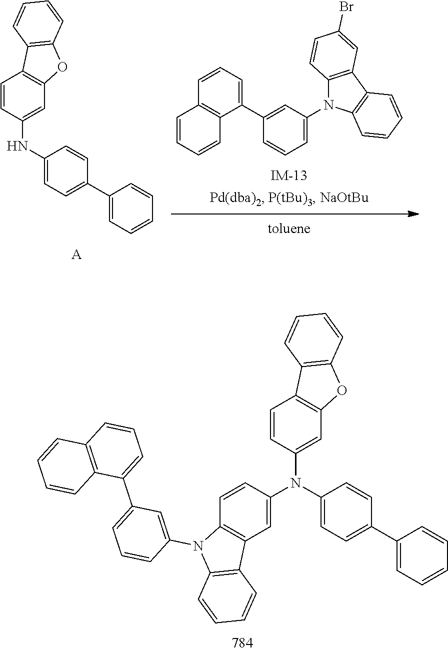

According to substantially the same method as the synthesis of Amine Compound 209, 7.0 g (yield 67%) of Amine Compound 784 was obtained from Intermediate Compound A (5.0 g) and Intermediate Compound IM-13 (6.7 g). The molecular weight of Amine Compound 784, measured by FAB-MS was 702.

### (13) Synthesis of Amine Compound 923

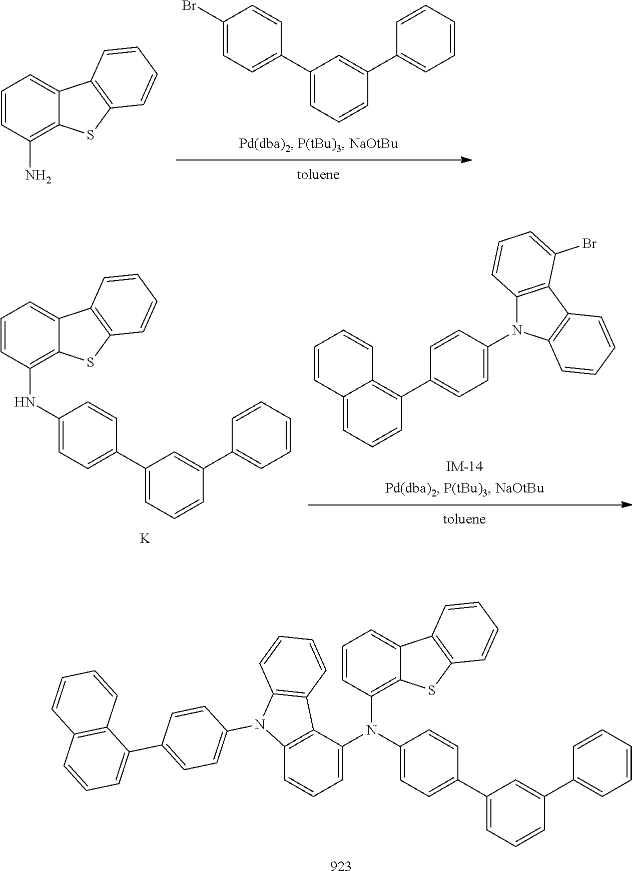

According to substantially the same method as the synthesis of Intermediate Compound A, 33.4 g (yield 78%) of Intermediate Compound K was obtained from dibenzo[b,d]thiophene-4-amine (20.0 g) and 4-bromo-1,1′:3′,1″-terphenyl (31.0 g). The molecular weight of Intermediate Compound K, measured by FAB-MS was 427.

According to substantially the same method as the synthesis of Amine Compound 209, 6.3 g (yield 68%) of Amine Compound 923 was obtained from Intermediate Compound K (5.0 g) and Intermediate Compound IM-14 (5.3 g). The molecular weight of Amine Compound 923, measured by FAB-MS was 795.

### (14) Synthesis of Amine Compound 968

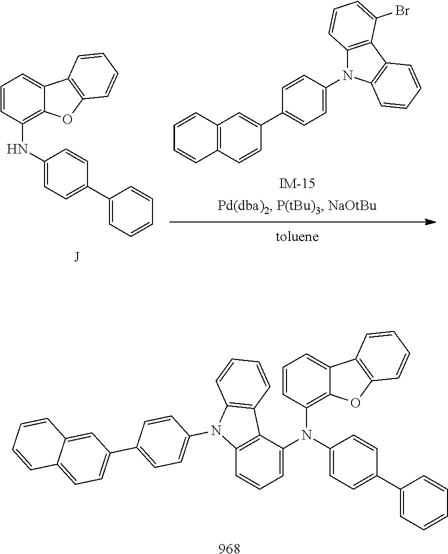

According to substantially the same method as the synthesis of Amine Compound 209, 6.1 g (yield 59%) of Amine Compound 968 was obtained from Intermediate Compound J (5.0 g) and Intermediate Compound IM-15 (6.7 g). The molecular weight of Amine Compound 968, measured by FAB-MS was 702.

### 2. Manufacture and Evaluation of Light Emitting Elements

A light emitting element of one or more embodiments, including the amine compound of one or more embodiments in a hole transport layer was manufactured by a method as described herein. Light emitting elements of Example 1 to Example 13 were manufactured utilizing the Example Compounds of Compounds 209, 295, 296, 300, 327, 480, 495, 504, 566, 584, 784, 923, and 968 as hole transport layer materials. Comparative Example 1 to Comparative Example 9 correspond to light emitting elements manufactured utilizing Comparative Compound X-1 to Comparative Compound X-9 as hole transport layer materials.

### Example Compounds

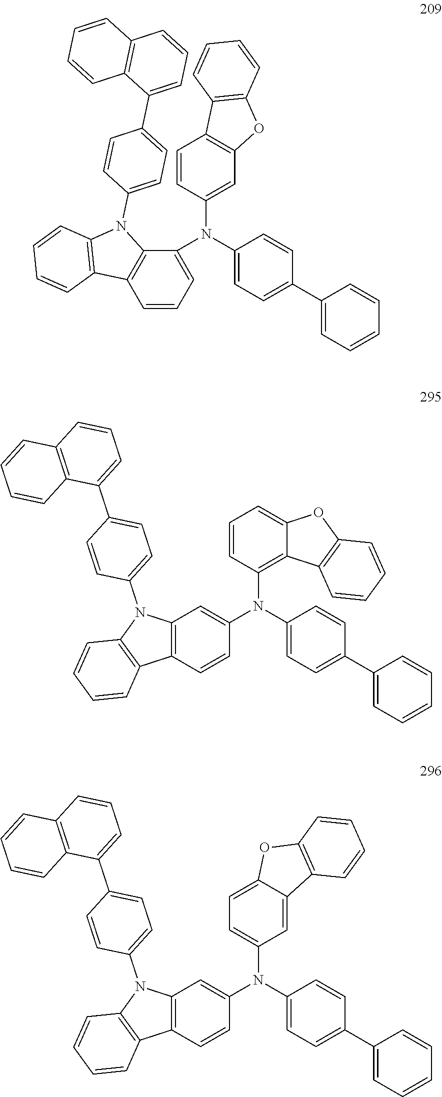 [SMILES: c1ccc(-c2ccc(N(c3ccc4c(c3)oc3ccccc34)c3cccc4c5ccccc5n(-c5ccc(-c6cccc7ccccc67)cc5)c34)cc2)cc1 ; c1ccc(-c2ccc(N(c3ccc4c5ccccc5n(-c5ccc(-c6cccc7ccccc67)cc5)c4c3)c3cccc4oc5ccccc5c34)cc2)cc1 ; c1ccc(-c2ccc(N(c3ccc4oc5ccccc5c4c3)c3ccc4c5ccccc5n(-c5ccc(-c6cccc7ccccc67)cc5)c4c3)cc2)cc1]

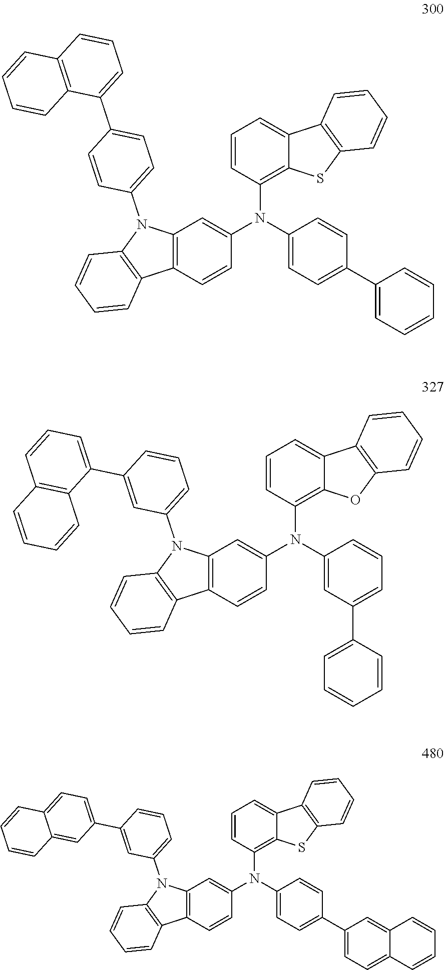 [SMILES: c1ccc(-c2ccc(N(c3ccc4c5ccccc5n(-c5ccc(-c6cccc7ccccc67)cc5)c4c3)c3cccc4c3sc3ccccc34)cc2)cc1 ; c1ccc(-c2cccc(N(c3ccc4c5ccccc5n(-c5cccc(-c6cccc7ccccc67)c5)c4c3)c3cccc4c3oc3ccccc34)c2)cc1 ; c1cc(-c2ccc3ccccc3c2)cc(-n2c3ccccc3c3ccc(N(c4ccc(-c5ccc6ccccc6c5)cc4)c4cccc5c4sc4ccccc45)cc32)c1]

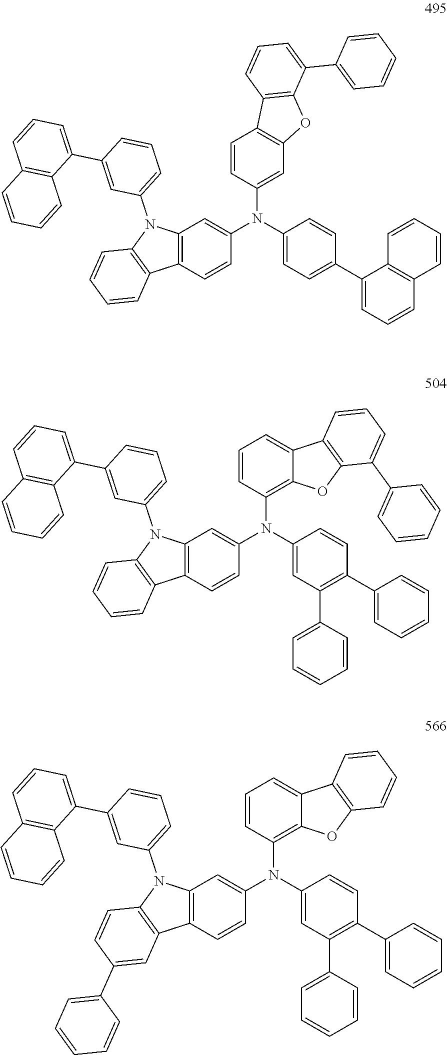 [SMILES: c1ccc(-c2cccc3c2oc2cc(N(c4ccc(-c5cccc6ccccc56)cc4)c4ccc5c6ccccc6n(-c6cccc(-c7cccc8ccccc78)c6)c5c4)ccc23)cc1 ; c1ccc(-c2ccc(N(c3ccc4c5ccccc5n(-c5cccc(-c6cccc7ccccc67)c5)c4c3)c3cccc4c3oc3c(-c5ccccc5)cccc34)cc2-c2ccccc2)cc1 ; c1ccc(-c2ccc3c(c2)c2ccc(N(c4ccc(-c5ccccc5)c(-c5ccccc5)c4)c4cccc5c4oc4ccccc45)cc2n3-c2cccc(-c3cccc4ccccc34)c2)cc1]

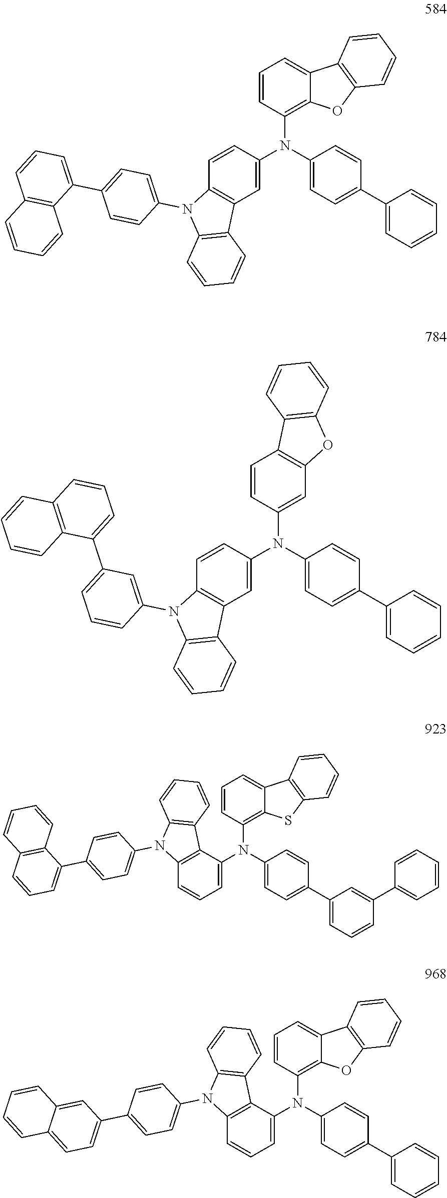 [SMILES: c1ccc(-c2ccc(N(c3ccc4c(c3)c3ccccc3n4-c3ccc(-c4cccc5ccccc45)cc3)c3cccc4c3oc3ccccc34)cc2)cc1 ; c1ccc(-c2ccc(N(c3ccc4c(c3)oc3ccccc34)c3ccc4c(c3)c3ccccc3n4-c3cccc(-c4cccc5ccccc45)c3)cc2)cc1 ; c1ccc(-c2cccc(-c3ccc(N(c4cccc5c4sc4ccccc45)c4cccc5c4c4ccccc4n5-c4ccc(-c5cccc6ccccc56)cc4)cc3)c2)cc1 ; c1ccc(-c2ccc(N(c3cccc4c3oc3ccccc34)c3cccc4c3c3ccccc3n4-c3ccc(-c4ccc5ccccc5c4)cc3)cc2)cc1]

### Comparative Compounds

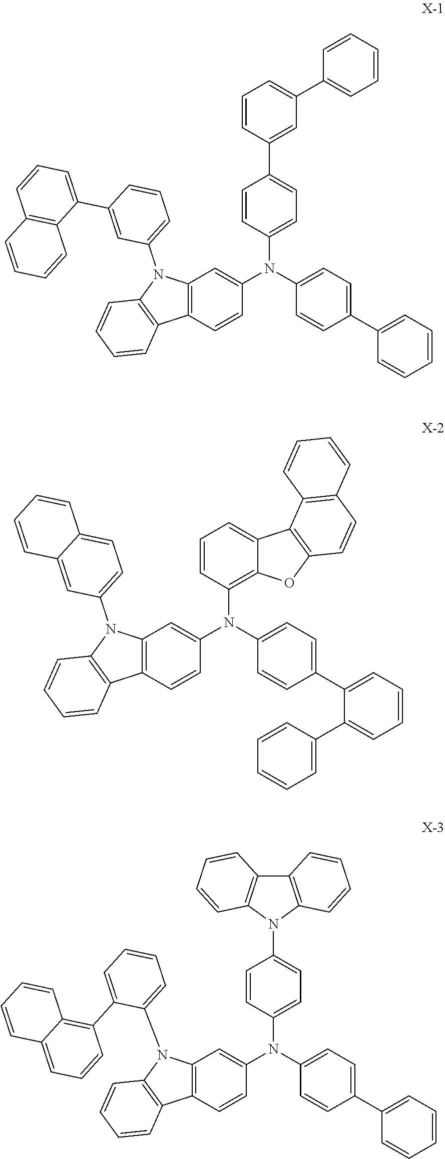 [SMILES: c1ccc(-c2ccc(N(c3ccc(-c4cccc(-c5ccccc5)c4)cc3)c3ccc4c5ccccc5n(-c5cccc(-c6cccc7ccccc67)c5)c4c3)cc2)cc1 ; c1ccc(-c2ccccc2-c2ccc(N(c3ccc4c5ccccc5n(-c5ccc6ccccc6c5)c4c3)c3cccc4c3oc3ccc5ccccc5c34)cc2)cc1 ; c1ccc(-c2ccc(N(c3ccc(-n4c5ccccc5c5ccccc54)cc3)c3ccc4c5ccccc5n(-c5ccccc5-c5cccc6ccccc56)c4c3)cc2)cc1]

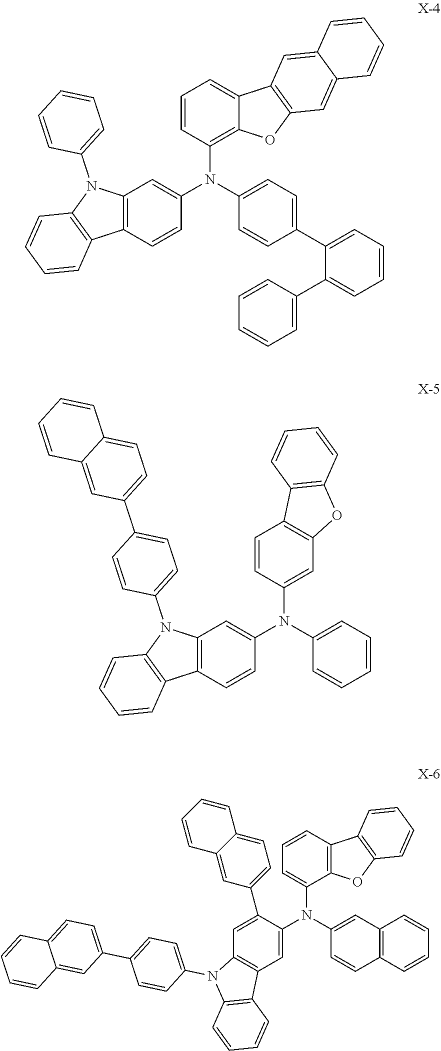 [SMILES: c1ccc(-c2ccccc2-c2ccc(N(c3ccc4c5ccccc5n(-c5ccccc5)c4c3)c3cccc4c3oc3cc5ccccc5cc34)cc2)cc1 ; c1ccc(N(c2ccc3c(c2)oc2ccccc23)c2ccc3c4ccccc4n(-c4ccc(-c5ccc6ccccc6c5)cc4)c3c2)cc1 ; c1ccc2cc(-c3ccc(-n4c5ccccc5c5cc(N(c6ccc7ccccc7c6)c6cccc7c6oc6ccccc67)c(-c6ccc7ccccc7c6)cc54)cc3)ccc2c1]

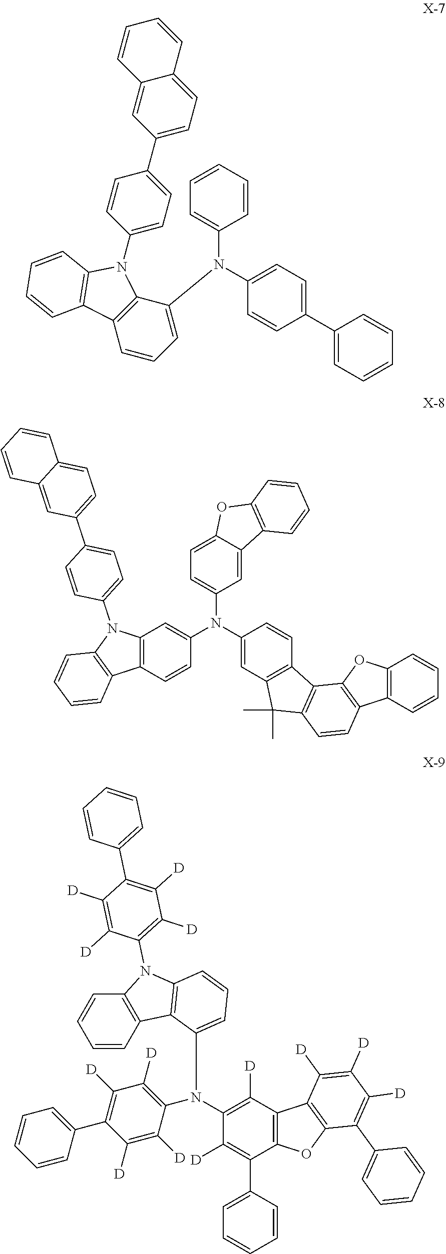 [SMILES: c1ccc(-c2ccc(N(c3ccccc3)c3cccc4c5ccccc5n(-c5ccc(-c6ccc7ccccc7c6)cc5)c34)cc2)cc1 ; CC1(C)c2cc(N(c3ccc4oc5ccccc5c4c3)c3ccc4c5ccccc5n(-c5ccc(-c6ccc7ccccc7c6)cc5)c4c3)ccc2-c2c1ccc1c2oc2ccccc21 ; [2H]c1c([2H])c(N(c2c([2H])c(-c3ccccc3)c3oc4c(-c5ccccc5)c([2H])c([2H])c([2H])c4c3c2[2H])c2cccc3c2c2ccccc2n3-c2c([2H])c([2H])c(-c3ccccc3)c([2H])c2[2H])c([2H])c([2H])c1-c1ccccc1]

### Manufacture of Light Emitting Elements

As a first electrode, an indium tin oxide (ITO) glass substrate with about 15 ohm per square centimeter (Ω/cm²) (about 150 nanometer (nm)) of Corning Co. was cut into a size of 50 millimeter (mm)×50 mm×0.7 mm, washed with isopropyl alcohol and ultrapure water, cleansed utilizing ultrasonic waves for about 5 minutes, exposed to UV for about 30 minutes and treated with ozone. Then, 4,4′,4″-tris{N,-(2-naphthyl)-N-phenylamino}-triphenylamine (2-TNATA) was vacuum deposited to a thickness of about 60 nm to form a hole injection layer. After that, the Example Compound or Comparative Compound was vacuum deposited to a thickness of about 30 nm to form a hole transport layer.

On the hole transport layer, a blue fluorescence host of 9,10-di(naphthalen-2-yl)anthracene (ADN) and a fluorescence dopant of 2,5,8,11-tetra-t-butylperylene (TBP) were co-deposited in a ratio (e.g., amount) of about 97:3 to form an emission layer with a thickness of about 25 nm.

On the emission layer, an electron transport layer was formed to a thickness of about 25 nm utilizing tris(8-hydroxyquinolino)aluminum (Alq₃), and then, an electron injection layer was formed to a thickness of about 1 nm by depositing LiF. On the electron injection layer, a second electrode was formed to a thickness of about 100 nm utilizing aluminum (Al).

In some embodiments, the compounds of the functional layers utilized for the manufacture of the light emitting elements are as follows.

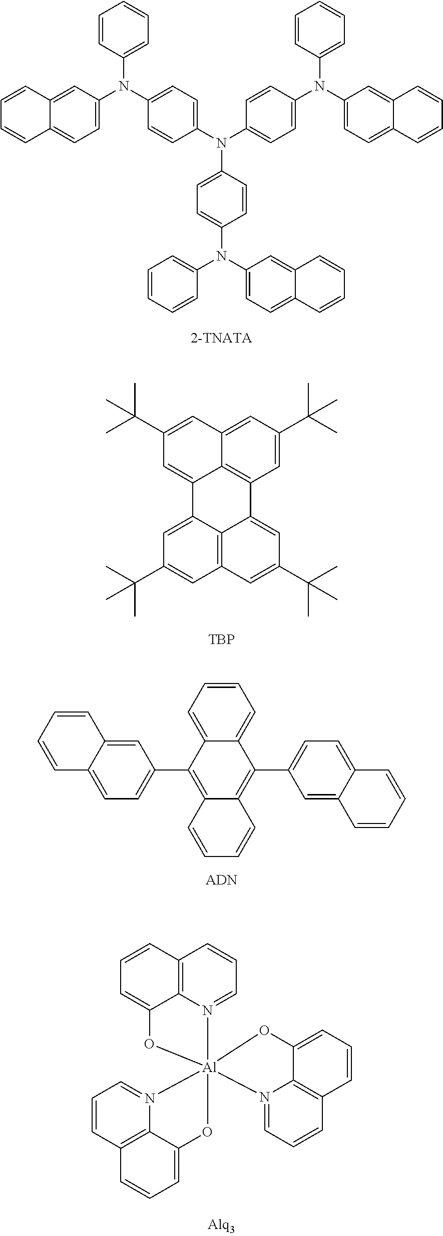

### Evaluation of Light Emitting Elements

Table 1 shows evaluation results on the light emitting elements of Examples 1 to 13 and Comparative Examples 1 to 9. In Table 1, the evaluation results of the emission efficiency and the lifetime of the light emitting elements manufactured are shown.

The evaluation results on the properties of the Examples and Comparative Examples, shown in Table 1 were conducted utilizing a Hamamatsu Photonics C9920-11 light distribution measurement system. The emission efficiency represents efficiency values at a current density of about 10 milliampere per square centimeter (mA/cm²), and the element lifetime (LT50) represents time taken for reducing an initial luminance of about 1000 candela per square meter (cd/m²) to a half luminance.

TABLE 1

| Element | | Emission | Element |
|---|---|---|---|
| manufacturing | Hole transport layer | efficiency | lifetime |
| example | materials | (@10 mA/cm²) | (LT50) |
| Example 1 | Amine Compound 209 | 109% | 110% |
| Example 2 | Amine Compound 295 | 109% | 110% |
| Example 3 | Amine Compound 296 | 107% | 110% |
| Example 4 | Amine Compound 300 | 107% | 120% |
| Example 5 | Amine Compound 327 | 108% | 120% |
| Example 6 | Amine Compound 480 | 106% | 130% |
| Example 7 | Amine Compound 495 | 102% | 150% |
| Example 8 | Amine Compound 504 | 108% | 120% |
| Example 9 | Amine Compound 566 | 108% | 120% |
| Example 10 | Amine Compound 584 | 103% | 110% |
| Example 11 | Amine Compound 784 | 102% | 120% |
| Example 12 | Amine Compound 923 | 109% | 110% |
| Example 13 | Amine Compound 968 | 108% | 120% |
| Comparative | Comparative | 100% | 100% |
| Example 1 | Compound X-1 | | |
| Comparative | Comparative | 101% | 95% |
| Example 2 | Compound X-2 | | |
| Comparative | Comparative | 100% | 80% |
| Example 3 | Compound X-3 | | |
| Comparative | Comparative | 102% | 60% |
| Example 4 | Compound X-4 | | |
| Comparative | Comparative | 98% | 100% |
| Example 5 | Compound X-5 | | |
| Comparative | Comparative | 100% | 40% |
| Example 6 | Compound X-6 | | |
| Comparative | Comparative | 101% | 50% |
| Example 7 | Compound X-7 | | |
| Comparative | Comparative | 98% | 70% |
| Example 8 | Compound X-8 | | |
| Comparative | Comparative | 100% | 50% |
| Example 9 | Compound X-9 | | |

Referring to the results of Table 1, it may be found that (e.g., most of) the light emitting elements of the Examples, utilizing the amine compounds according to one or more embodiments of the present disclosure as the hole transport layer materials, showed high emission efficiency when compared to the Comparative Examples. In some embodiments, it may be found that the light emitting elements of the Examples showed long lifetime characteristics when compared to the light emitting elements of the Comparative Examples.

For example, in the amine compound of one or more embodiments, it may be found that a carbazole moiety is directly connected with the nitrogen atom of an amine, and an aryl group of 10 to 30 ring-forming carbon atoms (for example, a naphthyl group) is connected at position 9 of the carbazole moiety via a phenylene linker, and electron tolerance was improved, and long lifetime characteristics were shown. In some embodiments, in the amine compounds of the Examples, it may be confirmed that because a dibenzoheterole such as a dibenzofuran moiety or dibenzothiophene moiety is directly bonded together with the nitrogen atom of an amine compound, excellent or suitable emission efficiency and long lifetime characteristics were shown.

In Comparative Compounds X-1 and X-7, because a dibenzofuran moiety or a dibenzothiophene moiety is not connected with the nitrogen atom of an amine, it may be found that emission efficiency was relatively low, and lifetime characteristics were degraded.

In Comparative Compound X-2, a naphthyl group is directly connected at position 9 of a carbazole moiety which is connected with the nitrogen atom of an amine, and a structure in which a benzene ring is additionally fused with a dibenzofuran moiety is included, and relatively low emission efficiency and lifetime characteristics were shown in contrast to the Examples.

In Comparative Compound X-3, a naphthyl group is connected (e.g., to a phenylene liker) at the ortho position with respect to the nitrogen atom of a carbazole moiety, and a dibenzofuran moiety or a dibenzothiophene moiety is not included, and relatively low emission efficiency and lifetime characteristics were shown in contrast to the Examples.

In Comparative Compound X-4, an aryl group of 10 to 30 ring-forming carbon atoms is not attached (e.g., introduced) to a phenyl group which is connected at position 9 of a carbazole moiety, and a structure in which a benzene ring is additionally fused with a dibenzofuran moiety, is included, and markedly degraded lifetime was shown in contrast to the Examples.

Comparative Compound X-5 corresponds to a case where the nitrogen atom of an amine is connected at position 2 (or position 6) of a carbazole moiety, while being connected at position 3 (or position 6) of a dibenzofuran moiety. However, Comparative Compound X-5 does not include a substituent connected with the dibenzofuran moiety, and degraded results of emission efficiency and lifetime were shown in contrast to the Examples.

Comparative Compound X-6 corresponds to a case where the nitrogen atom of an amine compound is connected at position 3 of a carbazole moiety. However, in Comparative Compound X-6, a naphthyl group is connected with the benzene ring of a carbazole moiety with which the nitrogen atom of an amine is connected, and markedly degraded lifetime results were shown in contrast to the Example compounds.

Comparative Compound X-8 essentially includes a heteroaryl group in which 9,9-dimethylfluorene is fused with benzofuran to form a ring, and lifetime was markedly degraded, and emission efficiency was low in contrast to the Examples.

In Comparative Compound X-9, a phenyl group of 6 ring-forming carbon atoms is connected at position 9 of a carbazole moiety via a phenylene linker, and the light emitting element of Comparative Example 9, utilizing Comparative Compound X-9 showed low emission efficiency and markedly degraded lifetime in contrast to the Examples.

The light emitting element of one or more embodiments includes the amine compound of one or more embodiments and may show high emission efficiency and long lifetime characteristics.

The amine compound of one or more embodiments has excellent or suitable hole transport properties and electron tolerance, and if applied to a light emitting element, the amine compound may contribute to the increase of the efficiency and lifetime of the light emitting element.

The display device of one or more embodiments includes the light emitting element and may show excellent or suitable display quality.

Although one or more embodiments of the present disclosure have been described, it is understood that the present disclosure should not be limited to these embodiments, but one or more suitable changes and modifications can be made by one ordinary skilled in the art within the spirit and scope of the present disclosure as hereinafter claimed and equivalents thereof.

Accordingly, the technical scope of the present disclosure is not intended to be limited to the contents set forth in the detailed description of the specification, but is intended to be defined by the appended claims, and equivalents thereof.

What is claimed is:

1. A light emitting element, comprising:
a first electrode;
a second electrode on the first electrode; and
at least one functional layer between the first electrode and the second electrode,
the at least one functional layer comprising an amine compound, the amine compound is represented by Formula 1: 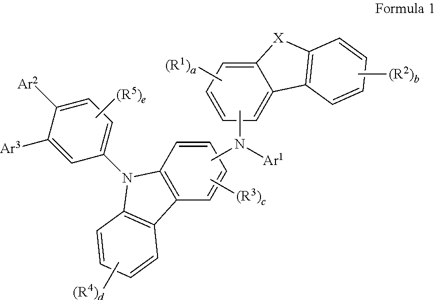
in Formula 1,
X is O or S,
Ar¹ is a substituted or unsubstituted aryl group of 6 to 30 ring-forming carbon atoms, or a substituted or unsubstituted heteroaryl group of 2 to 30 ring-forming carbon atoms,
Ar² and Ar³ are each independently a hydrogen atom, a deuterium atom, or a substituted or unsubstituted aryl group of 6 to 30 ring-forming carbon atoms, where at least one selected from among Ar² and Ar³ is a substituted or unsubstituted aryl group of 10 to 30 ring-forming carbon atoms,
R¹ to R⁵ are each independently a hydrogen atom, a deuterium atom, a halogen atom, a substituted or unsubstituted alkyl group of 1 to 15 carbon atoms, a substituted or unsubstituted aryl group of 6 to 30 ring-forming carbon atoms, or a substituted or unsubstituted heteroaryl group of 2 to 30 ring-forming carbon atoms,
“a”, “c” and “e” are each independently an integer of 0 to 3,
“b” and “d” are each independently an integer of 0 to 4,
when
a nitrogen atom of the amine compound is connected with position 2 or position 6 of a carbazole moiety, and is connected with position 3 or position 6 of a dibenzothiophene moiety or a dibenzofuran moiety, or
the nitrogen atom of the amine compound is connected with position 4 or position 5 of the carbazole moiety, and is connected with position 3 or position 7 of the dibenzothiophene moiety or the dibenzofuran moiety,
then
a+b≥1, and at least one selected from among R¹ and R² is not a hydrogen atom, and
when the nitrogen atom of the amine compound is connected with position 3 of the carbazole moiety, then R³ is a hydrogen atom or a deuterium atom, and
Ar¹ comprises a moiety of 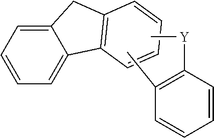
and Y is O or S, is excluded.

2. The light emitting element of claim 1, wherein
the at least one functional layer comprises an emission layer, a hole transport region between the first electrode and the emission layer, and an electron transport region between the emission layer and the second electrode, and
the hole transport region comprises the amine compound.

3. The light emitting element of claim 2, wherein the hole transport region comprises at least one selected from among a hole injection layer, a hole transport layer and an electron blocking layer, and
at least one selected from among the hole injection layer, the hole transport layer and the electron blocking layer comprises the amine compound.

4. The light emitting element of claim 2, wherein
the hole transport region comprises a hole injection layer on the first electrode, and a hole transport layer on the hole injection layer, and
the hole transport layer comprises the amine compound.

5. The light emitting element of claim 1, wherein the amine compound is a monoamine compound.

6. The light emitting element of claim 1, wherein the amine compound is represented by Formula 2-1 or Formula 2-2: 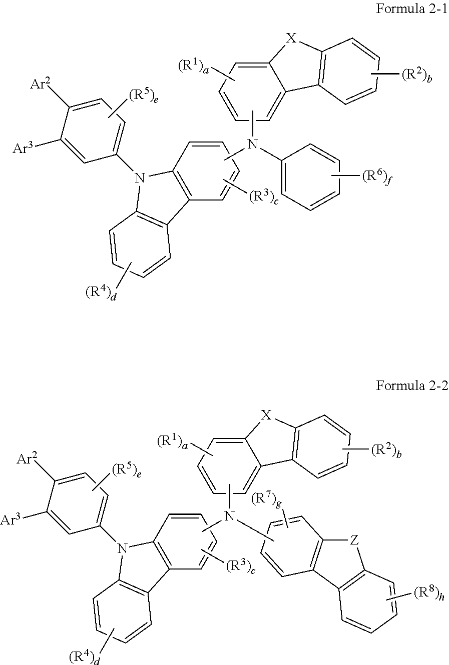
in Formula 2-1,
R⁶ is a hydrogen atom, a deuterium atom, a substituted or unsubstituted aryl group of 6 to 30 ring-forming carbon atoms, or a substituted or unsubstituted heteroaryl group of 2 to 30 ring-forming carbon atoms, and
“f” is an integer of 0 to 5,
in Formula 2-2,
Z is O or S,
R⁷ and R⁸ are each independently a hydrogen atom or a deuterium atom,
“g” is an integer of 0 to 3, and “h” is an integer of 0 to 4, and
in Formula 2-1 and Formula 2-2,
X, Ar², Ar³, R¹ to R⁵, and “a” to “e” are as defined in Formula 1.

7. The light emitting element of claim 1, wherein at least one selected from among Ar² and Ar³ is a substituted or unsubstituted naphthyl group, a substituted or unsubstituted phenanthrenyl group, a substituted or unsubstituted triphenylenyl group, or a substituted or unsubstituted pyrenyl group.

8. The light emitting element of claim 7, wherein the amine compound is represented by Formula 3-1 or Formula 3-2: 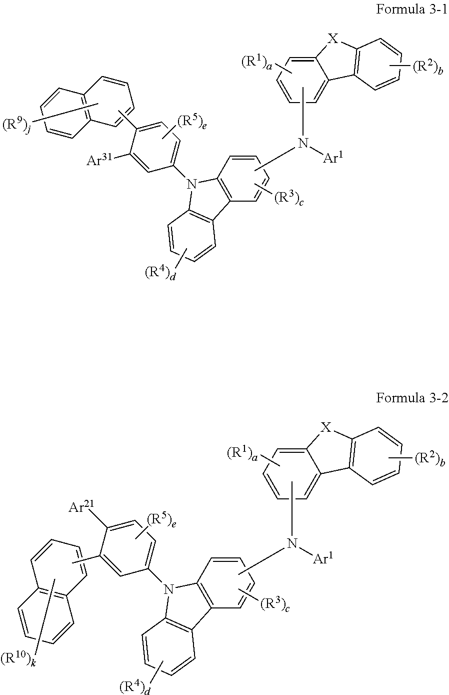
in Formula 3-1 and Formula 3-2,
R⁹, R¹⁰, Ar²¹ and Ar³¹ are each independently a hydrogen atom, a deuterium atom, or a substituted or unsubstituted aryl group of 6 to 30 ring-forming carbon atoms,
“i” and “k” are each independently an integer of 0 to 7, and
X, Ar¹, R¹ to R⁵, and “a” to “e” are as defined in Formula 1.

9. The light emitting element of claim 1, wherein R¹ to R⁵ are each independently a hydrogen atom, a deuterium atom, a halogen atom, a substituted or unsubstituted methyl group, or a substituted or unsubstituted phenyl group.

10. The light emitting element of claim 1, wherein the amine compound is represented by any one selected from among Formula 4-1 to Formula 4-4: 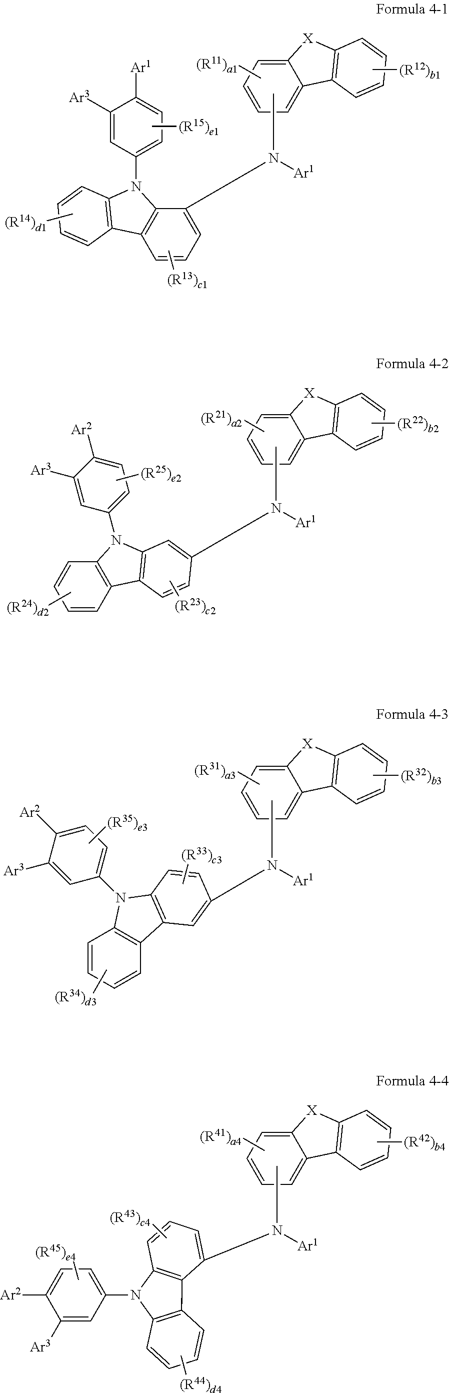
in Formula 4-1 to Formula 4-4,
R¹¹ to R¹³, R²¹ to R²³, R³¹ to R³³, R⁴¹, and R⁴² are each independently a hydrogen atom, a deuterium atom, or a substituted or unsubstituted aryl group of 6 to ring-forming carbon atoms,
R¹⁴, R²⁴, R³⁴, and R⁴⁴ are each independently a hydrogen atom, a deuterium atom, a halogen atom, a substituted or unsubstituted alkyl group of 1 to 15 carbon atoms, or a substituted or unsubstituted aryl group of 6 to 30 ring-forming carbon atoms,
R¹⁵, R²⁵, R³⁵, R⁴³ and R⁴⁵ are each independently a hydrogen atom, or a deuterium atom,
a1 to a4, c1 to c4, and e1 to e4 are each independently an integer of 0 to 3,
b1 to b4, and d1 to d4 are each independently an integer of 0 to 4,
X, and Ar¹ to Ar³ are as defined in Formula 1,
in Formula 4-2 when the nitrogen atom of the amine compound is connected with position 3 or position 6 of a dibenzothiophene moiety or a dibenzofuran moiety, then a2+b2≥1, and at least one selected from among R²¹ and R²² is a substituted or unsubstituted aryl group of 6 to 30 ring-forming carbon atoms, and
in Formula 4-4 when the nitrogen atom of the amine compound is connected with position 3 or position 6 of a dibenzothiophene moiety or a dibenzofuran moiety, then a4+b4≥1, and at least one selected from among R⁴¹ and R⁴² is a substituted or unsubstituted aryl group of 6 to 30 ring-forming carbon atoms.

11. The light emitting element of claim 1, wherein at least one selected from among Ar² and Ar³ is represented by any one selected from among substituents in Substituent Group 1: 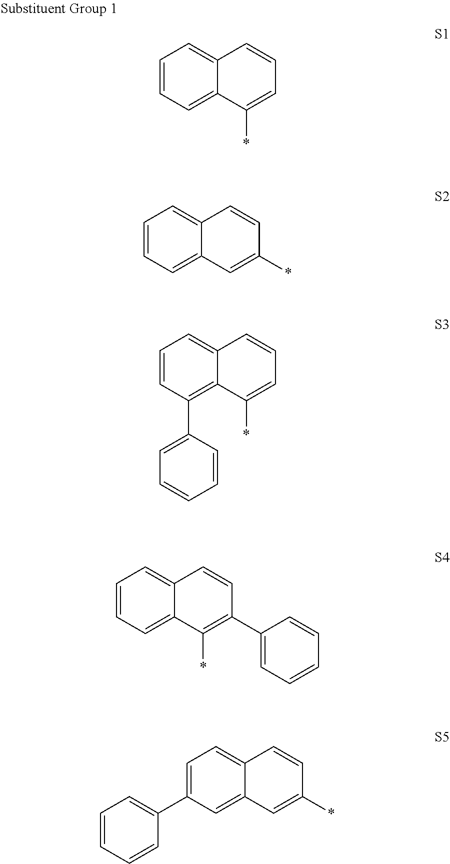 [SMILES: *c1cccc2ccccc12 ; *c1ccc2ccccc2c1 ; *c1cccc2cccc(-c3ccccc3)c12 ; *c1c(-c2ccccc2)ccc2ccccc12 ; *c1ccc2ccc(-c3ccccc3)cc2c1] 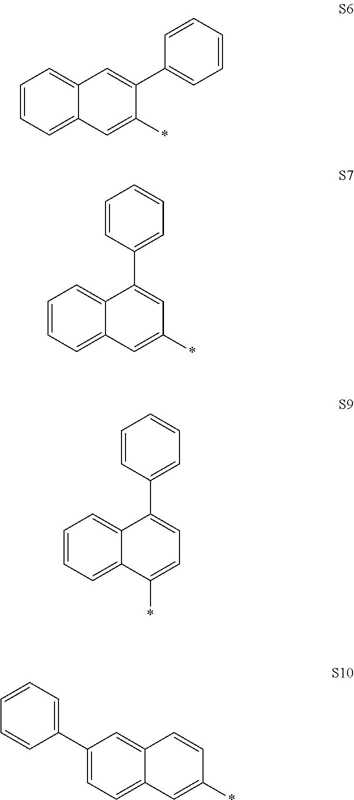 [SMILES: *c1cc2ccccc2cc1-c1ccccc1 ; *c1cc(-c2ccccc2)c2ccccc2c1 ; *c1ccc(-c2ccccc2)c2ccccc12 ; *c1ccc2cc(-c3ccccc3)ccc2c1] 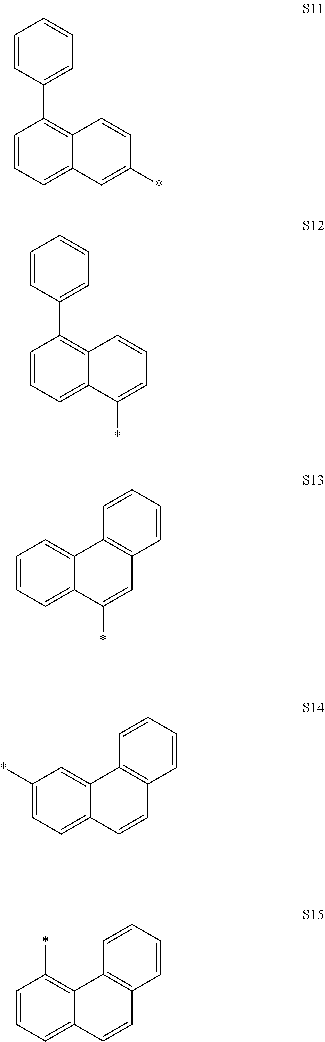 [SMILES: *c1ccc2c(-c3ccccc3)cccc2c1 ; *c1cccc2c(-c3ccccc3)cccc12 ; *c1cc2ccccc2c2ccccc12 ; *c1ccc2ccc3ccccc3c2c1 ; *c1cccc2ccc3ccccc3c12] 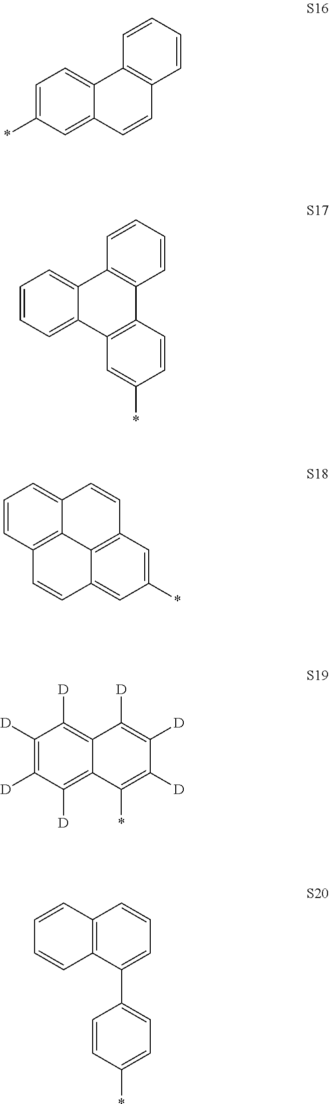 [SMILES: *c1ccc2c(ccc3ccccc32)c1 ; *c1ccc2c3ccccc3c3ccccc3c2c1 ; *c1cc2ccc3cccc4ccc(c1)c2c34 ; *c1c([2H])c([2H])c([2H])c2c([2H])c([2H])c([2H])c([2H])c12 ; *c1ccc(-c2cccc3ccccc23)cc1] 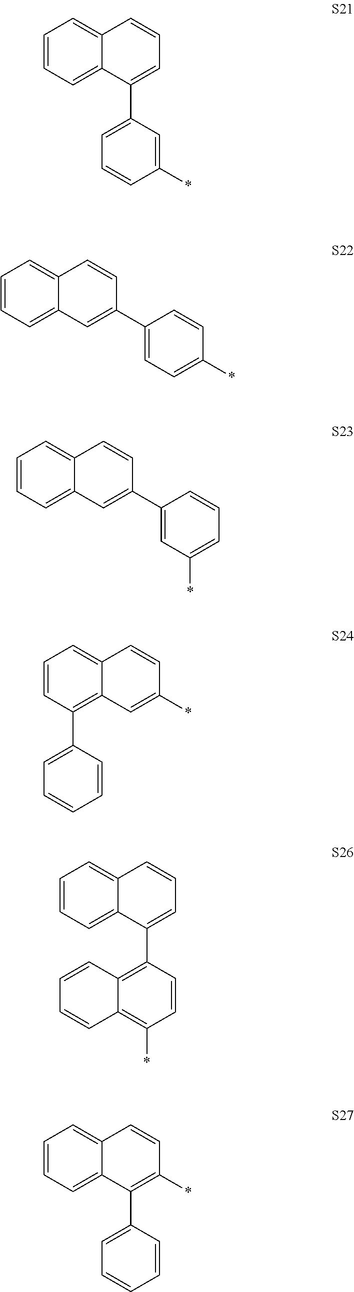 [SMILES: *c1cccc(-c2cccc3ccccc23)c1 ; *c1ccc(-c2ccc3ccccc3c2)cc1 ; *c1cccc(-c2ccc3ccccc3c2)c1 ; *c1ccc2cccc(-c3ccccc3)c2c1 ; *c1ccc(-c2cccc3ccccc23)c2ccccc12 ; *c1ccc2ccccc2c1-c1ccccc1]
in Substituent Group 1, D is a deuterium atom.

12. The light emitting element of claim 1, wherein at least one selected from among Ar¹ to Ar³, and R¹ to R⁵ comprises a deuterium atom, or a substituent comprising a deuterium atom.

13. The light emitting element of claim 1, wherein the amine compound is represented by any one selected from among compounds in Compound Group 1: 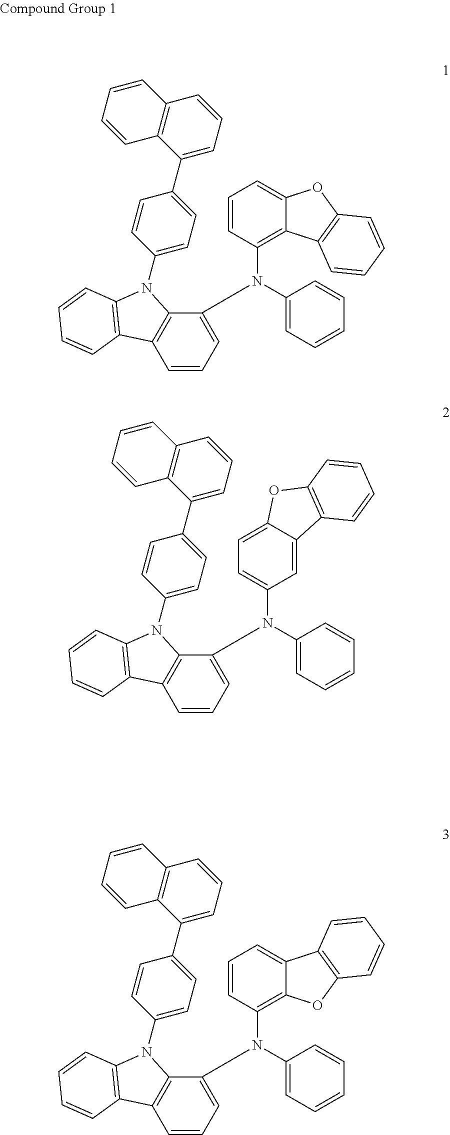 [SMILES: c1ccc(N(c2cccc3oc4ccccc4c23)c2cccc3c4ccccc4n(-c4ccc(-c5cccc6ccccc56)cc4)c23)cc1 ; c1ccc(N(c2ccc3oc4ccccc4c3c2)c2cccc3c4ccccc4n(-c4ccc(-c5cccc6ccccc56)cc4)c23)cc1 ; c1ccc(N(c2cccc3c2oc2ccccc23)c2cccc3c4ccccc4n(-c4ccc(-c5cccc6ccccc56)cc4)c23)cc1] 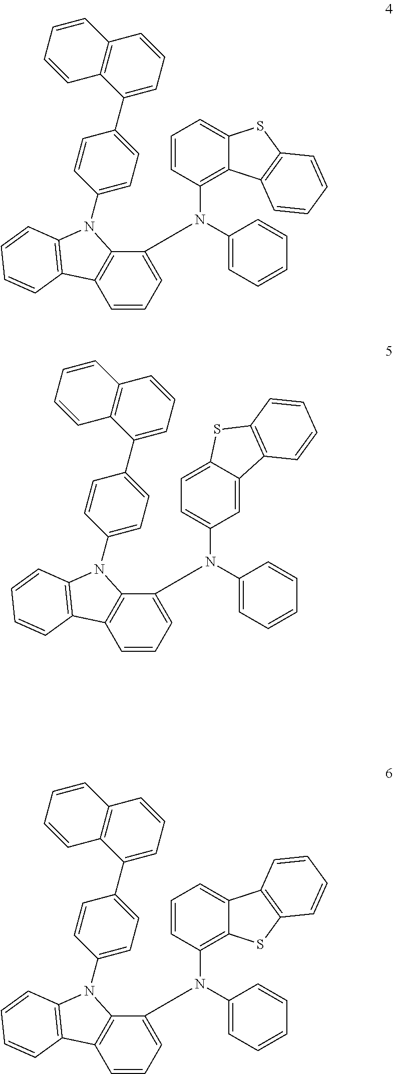 [SMILES: c1ccc(N(c2cccc3sc4ccccc4c23)c2cccc3c4ccccc4n(-c4ccc(-c5cccc6ccccc56)cc4)c23)cc1 ; c1ccc(N(c2ccc3sc4ccccc4c3c2)c2cccc3c4ccccc4n(-c4ccc(-c5cccc6ccccc56)cc4)c23)cc1 ; c1ccc(N(c2cccc3c2sc2ccccc23)c2cccc3c4ccccc4n(-c4ccc(-c5cccc6ccccc56)cc4)c23)cc1] 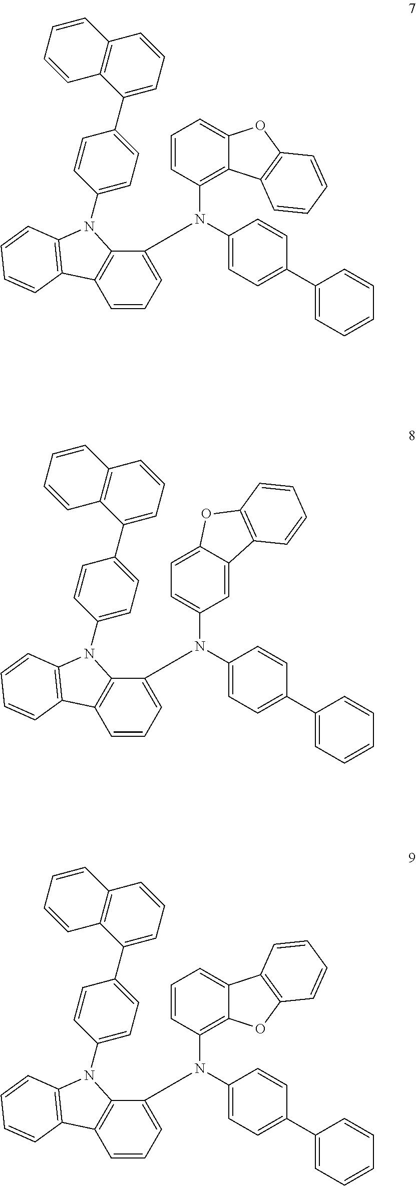 [SMILES: c1ccc(-c2ccc(N(c3cccc4oc5ccccc5c34)c3cccc4c5ccccc5n(-c5ccc(-c6cccc7ccccc67)cc5)c34)cc2)cc1 ; c1ccc(-c2ccc(N(c3ccc4oc5ccccc5c4c3)c3cccc4c5ccccc5n(-c5ccc(-c6cccc7ccccc67)cc5)c34)cc2)cc1 ; c1ccc(-c2ccc(N(c3cccc4c3oc3ccccc34)c3cccc4c5ccccc5n(-c5ccc(-c6cccc7ccccc67)cc5)c34)cc2)cc1] 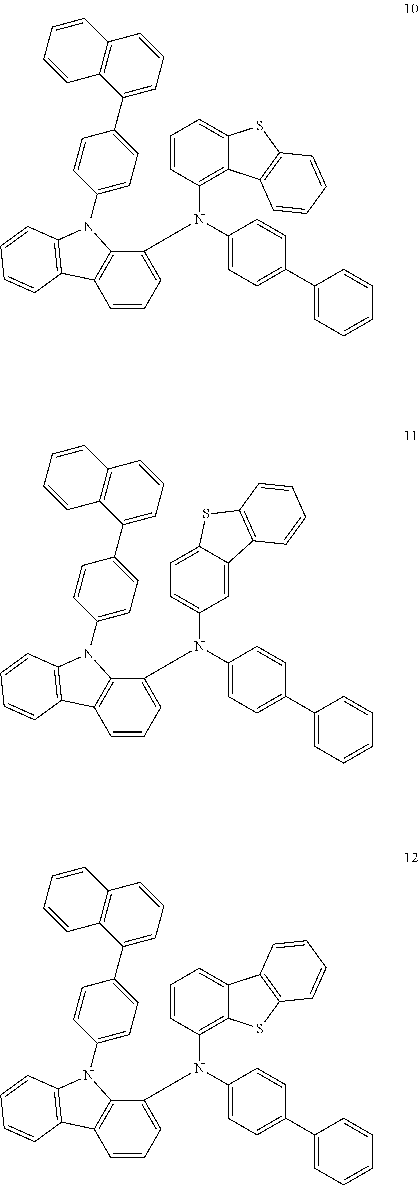 [SMILES: c1ccc(-c2ccc(N(c3cccc4sc5ccccc5c34)c3cccc4c5ccccc5n(-c5ccc(-c6cccc7ccccc67)cc5)c34)cc2)cc1 ; c1ccc(-c2ccc(N(c3ccc4sc5ccccc5c4c3)c3cccc4c5ccccc5n(-c5ccc(-c6cccc7ccccc67)cc5)c34)cc2)cc1 ; c1ccc(-c2ccc(N(c3cccc4c3sc3ccccc34)c3cccc4c5ccccc5n(-c5ccc(-c6cccc7ccccc67)cc5)c34)cc2)cc1] 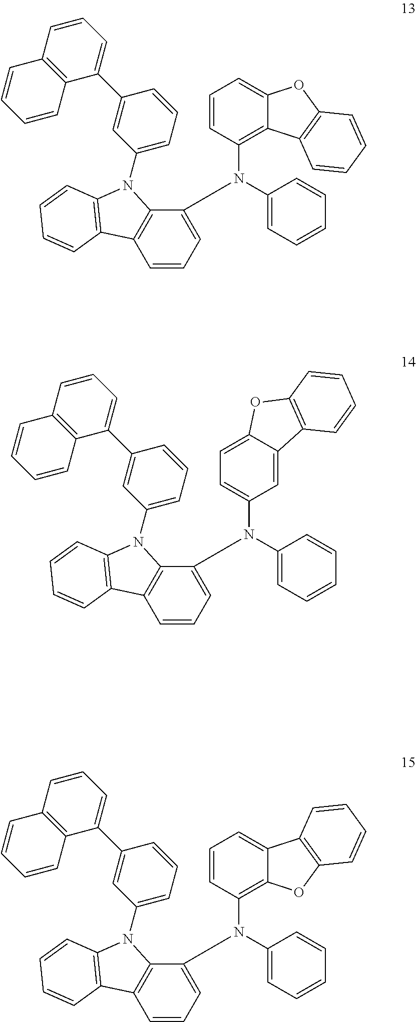 [SMILES: c1ccc(N(c2cccc3oc4ccccc4c23)c2cccc3c4ccccc4n(-c4cccc(-c5cccc6ccccc56)c4)c23)cc1 ; c1ccc(N(c2ccc3oc4ccccc4c3c2)c2cccc3c4ccccc4n(-c4cccc(-c5cccc6ccccc56)c4)c23)cc1 ; c1ccc(N(c2cccc3c2oc2ccccc23)c2cccc3c4ccccc4n(-c4cccc(-c5cccc6ccccc56)c4)c23)cc1] 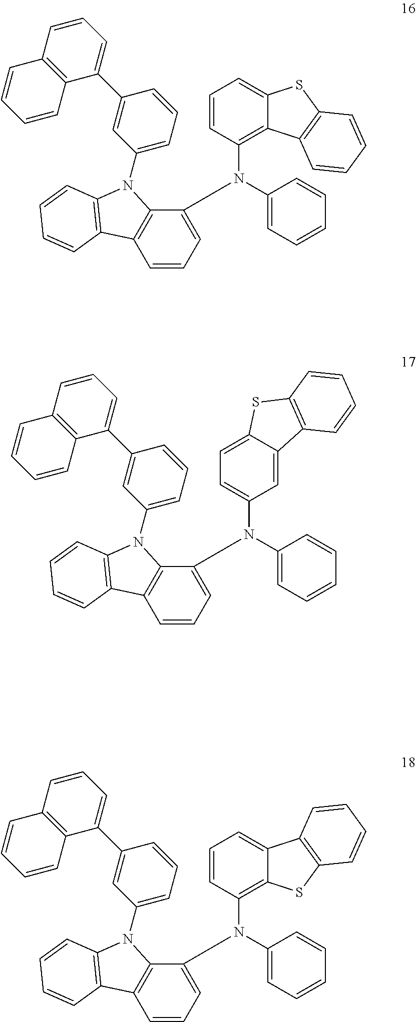 [SMILES: c1ccc(N(c2cccc3sc4ccccc4c23)c2cccc3c4ccccc4n(-c4cccc(-c5cccc6ccccc56)c4)c23)cc1 ; c1ccc(N(c2ccc3sc4ccccc4c3c2)c2cccc3c4ccccc4n(-c4cccc(-c5cccc6ccccc56)c4)c23)cc1 ; c1ccc(N(c2cccc3c2sc2ccccc23)c2cccc3c4ccccc4n(-c4cccc(-c5cccc6ccccc56)c4)c23)cc1] 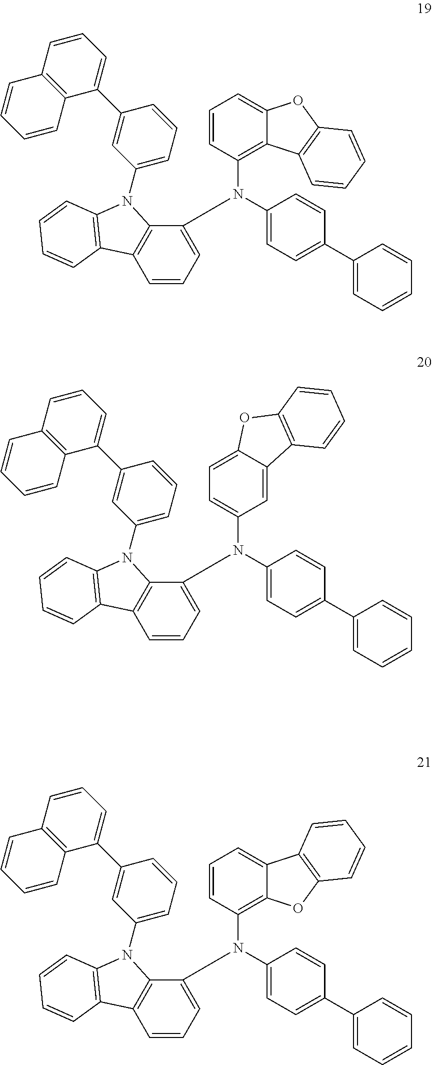 [SMILES: c1ccc(-c2ccc(N(c3cccc4oc5ccccc5c34)c3cccc4c5ccccc5n(-c5cccc(-c6cccc7ccccc67)c5)c34)cc2)cc1 ; c1ccc(-c2ccc(N(c3ccc4oc5ccccc5c4c3)c3cccc4c5ccccc5n(-c5cccc(-c6cccc7ccccc67)c5)c34)cc2)cc1 ; c1ccc(-c2ccc(N(c3cccc4c3oc3ccccc34)c3cccc4c5ccccc5n(-c5cccc(-c6cccc7ccccc67)c5)c34)cc2)cc1] 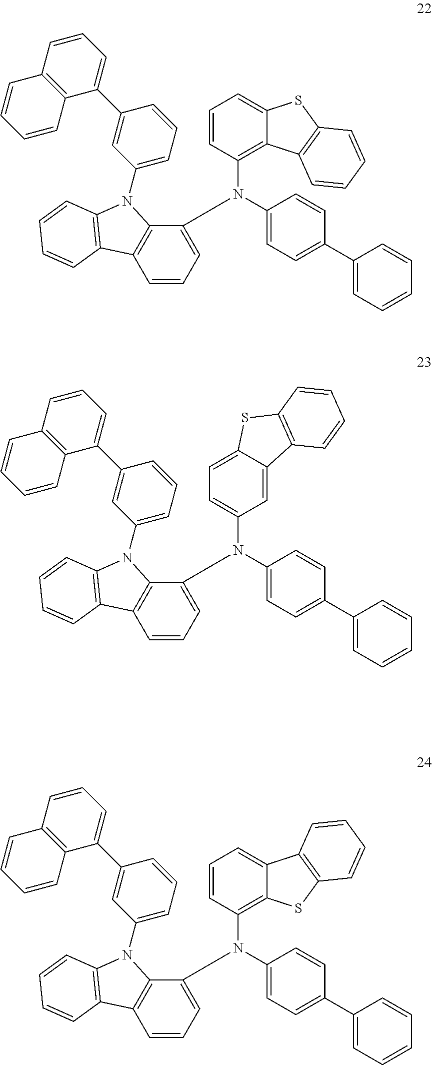 [SMILES: c1ccc(-c2ccc(N(c3cccc4sc5ccccc5c34)c3cccc4c5ccccc5n(-c5cccc(-c6cccc7ccccc67)c5)c34)cc2)cc1 ; c1ccc(-c2ccc(N(c3ccc4sc5ccccc5c4c3)c3cccc4c5ccccc5n(-c5cccc(-c6cccc7ccccc67)c5)c34)cc2)cc1 ; c1ccc(-c2ccc(N(c3cccc4c3sc3ccccc34)c3cccc4c5ccccc5n(-c5cccc(-c6cccc7ccccc67)c5)c34)cc2)cc1] 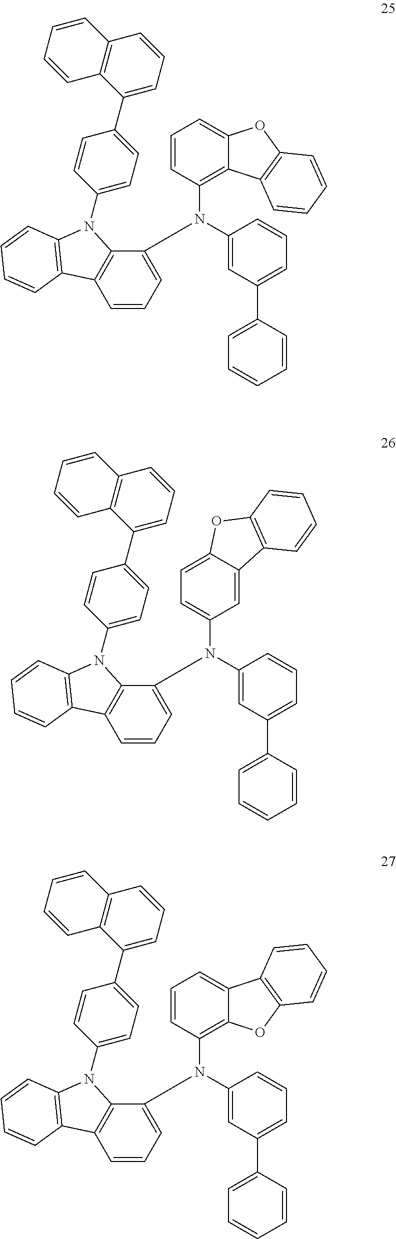 [SMILES: c1ccc(-c2cccc(N(c3cccc4oc5ccccc5c34)c3cccc4c5ccccc5n(-c5ccc(-c6cccc7ccccc67)cc5)c34)c2)cc1 ; c1ccc(-c2cccc(N(c3ccc4oc5ccccc5c4c3)c3cccc4c5ccccc5n(-c5ccc(-c6cccc7ccccc67)cc5)c34)c2)cc1 ; c1ccc(-c2cccc(N(c3cccc4c3oc3ccccc34)c3cccc4c5ccccc5n(-c5ccc(-c6cccc7ccccc67)cc5)c34)c2)cc1] 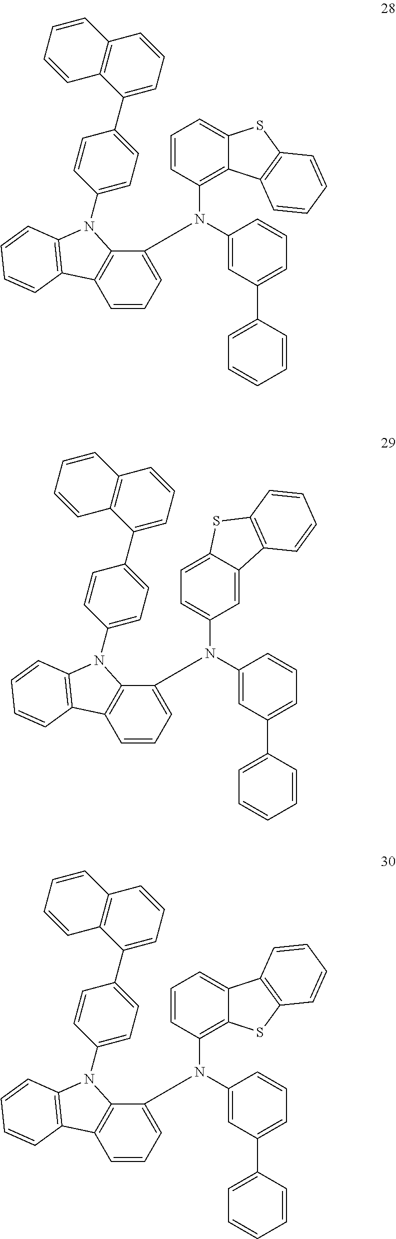 [SMILES: c1ccc(-c2cccc(N(c3cccc4sc5ccccc5c34)c3cccc4c5ccccc5n(-c5ccc(-c6cccc7ccccc67)cc5)c34)c2)cc1 ; c1ccc(-c2cccc(N(c3ccc4sc5ccccc5c4c3)c3cccc4c5ccccc5n(-c5ccc(-c6cccc7ccccc67)cc5)c34)c2)cc1 ; c1ccc(-c2cccc(N(c3cccc4c3sc3ccccc34)c3cccc4c5ccccc5n(-c5ccc(-c6cccc7ccccc67)cc5)c34)c2)cc1] 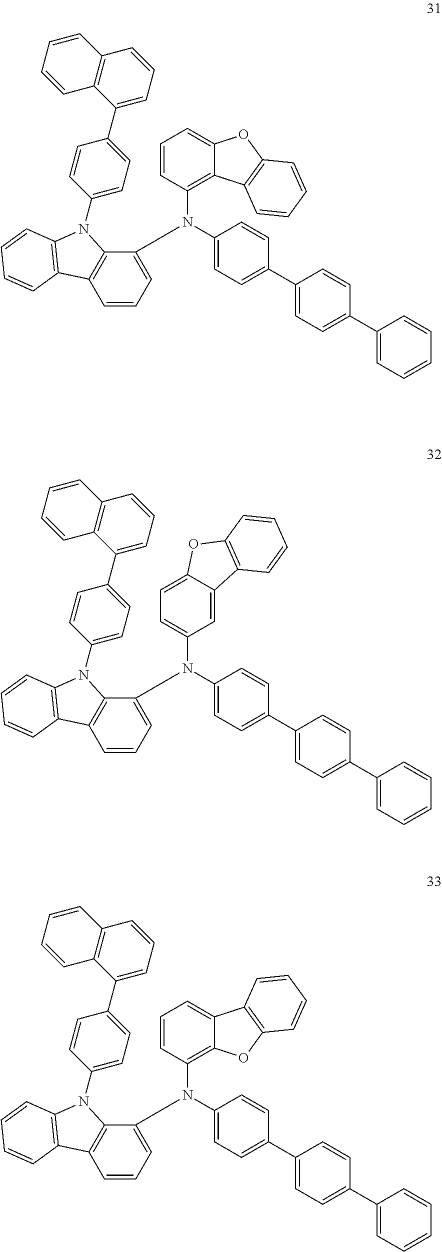 [SMILES: c1ccc(-c2ccc(-c3ccc(N(c4cccc5oc6ccccc6c45)c4cccc5c6ccccc6n(-c6ccc(-c7cccc8ccccc78)cc6)c45)cc3)cc2)cc1 ; c1ccc(-c2ccc(-c3ccc(N(c4ccc5oc6ccccc6c5c4)c4cccc5c6ccccc6n(-c6ccc(-c7cccc8ccccc78)cc6)c45)cc3)cc2)cc1 ; c1ccc(-c2ccc(-c3ccc(N(c4cccc5c4oc4ccccc45)c4cccc5c6ccccc6n(-c6ccc(-c7cccc8ccccc78)cc6)c45)cc3)cc2)cc1] 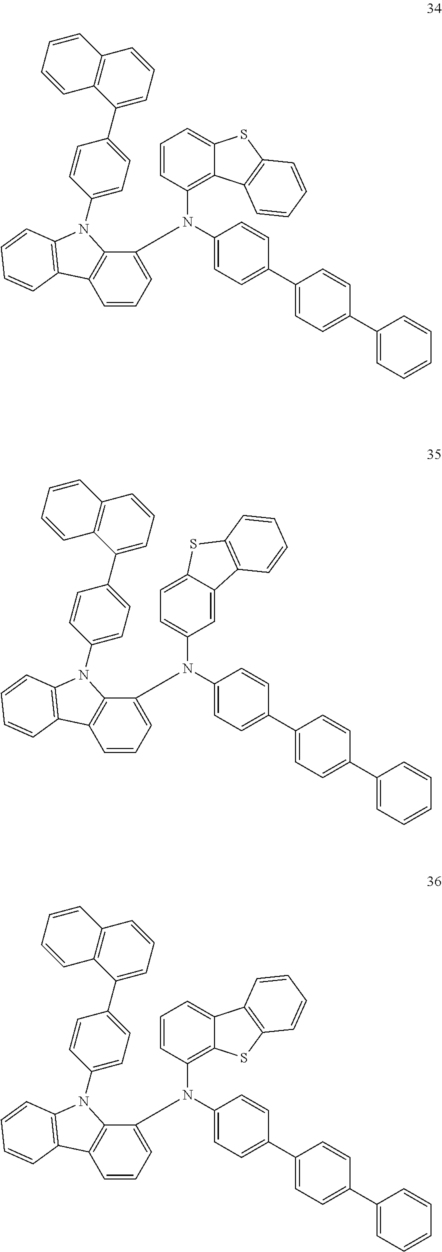 [SMILES: c1ccc(-c2ccc(-c3ccc(N(c4cccc5sc6ccccc6c45)c4cccc5c6ccccc6n(-c6ccc(-c7cccc8ccccc78)cc6)c45)cc3)cc2)cc1 ; c1ccc(-c2ccc(-c3ccc(N(c4ccc5sc6ccccc6c5c4)c4cccc5c6ccccc6n(-c6ccc(-c7cccc8ccccc78)cc6)c45)cc3)cc2)cc1 ; c1ccc(-c2ccc(-c3ccc(N(c4cccc5c4sc4ccccc45)c4cccc5c6ccccc6n(-c6ccc(-c7cccc8ccccc78)cc6)c45)cc3)cc2)cc1] 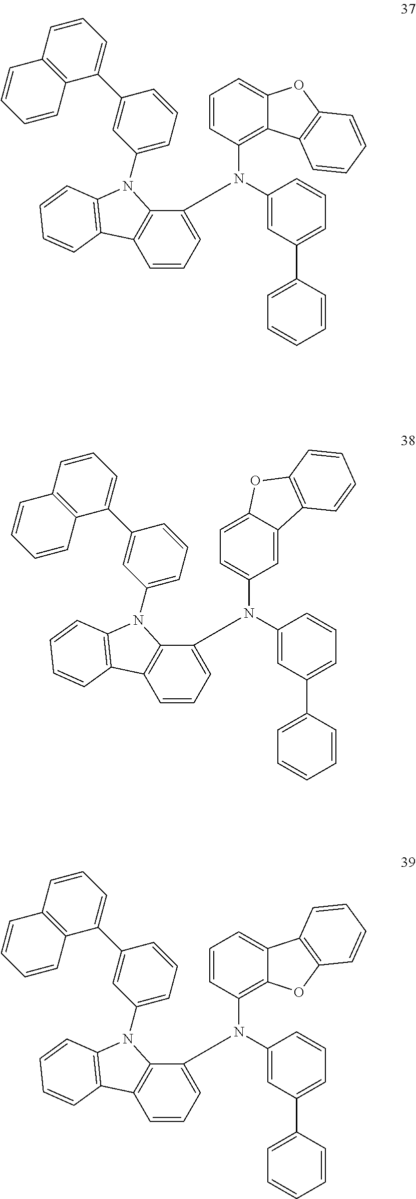 [SMILES: c1ccc(-c2cccc(N(c3cccc4oc5ccccc5c34)c3cccc4c5ccccc5n(-c5cccc(-c6cccc7ccccc67)c5)c34)c2)cc1 ; c1ccc(-c2cccc(N(c3ccc4oc5ccccc5c4c3)c3cccc4c5ccccc5n(-c5cccc(-c6cccc7ccccc67)c5)c34)c2)cc1 ; c1ccc(-c2cccc(N(c3cccc4c3oc3ccccc34)c3cccc4c5ccccc5n(-c5cccc(-c6cccc7ccccc67)c5)c34)c2)cc1] 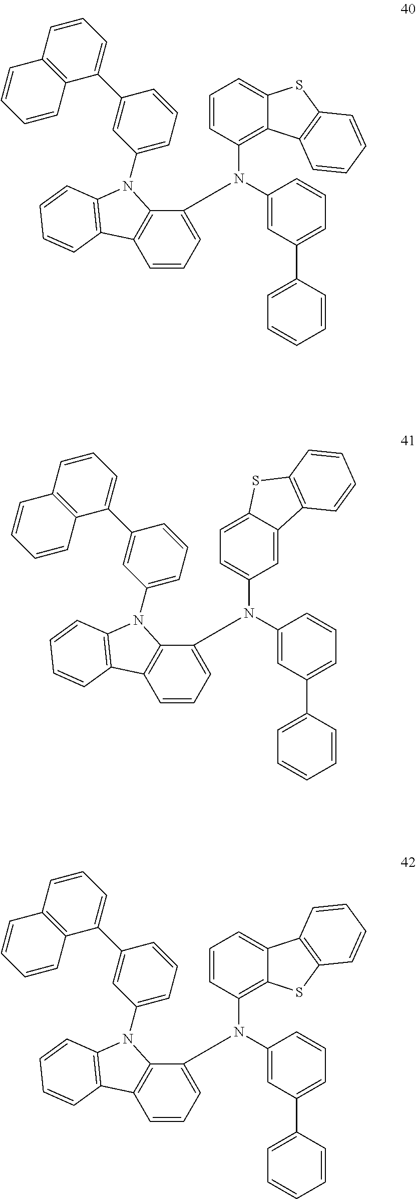 [SMILES: c1ccc(-c2cccc(N(c3cccc4sc5ccccc5c34)c3cccc4c5ccccc5n(-c5cccc(-c6cccc7ccccc67)c5)c34)c2)cc1 ; c1ccc(-c2cccc(N(c3ccc4sc5ccccc5c4c3)c3cccc4c5ccccc5n(-c5cccc(-c6cccc7ccccc67)c5)c34)c2)cc1 ; c1ccc(-c2cccc(N(c3cccc4c3sc3ccccc34)c3cccc4c5ccccc5n(-c5cccc(-c6cccc7ccccc67)c5)c34)c2)cc1] 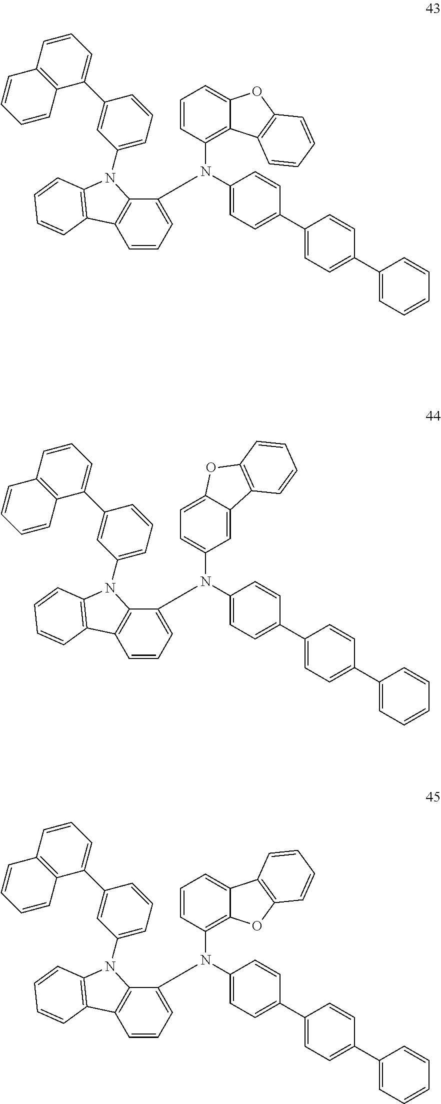 [SMILES: c1ccc(-c2ccc(-c3ccc(N(c4cccc5oc6ccccc6c45)c4cccc5c6ccccc6n(-c6cccc(-c7cccc8ccccc78)c6)c45)cc3)cc2)cc1 ; c1ccc(-c2ccc(-c3ccc(N(c4ccc5oc6ccccc6c5c4)c4cccc5c6ccccc6n(-c6cccc(-c7cccc8ccccc78)c6)c45)cc3)cc2)cc1 ; c1ccc(-c2ccc(-c3ccc(N(c4cccc5c4oc4ccccc45)c4cccc5c6ccccc6n(-c6cccc(-c7cccc8ccccc78)c6)c45)cc3)cc2)cc1] 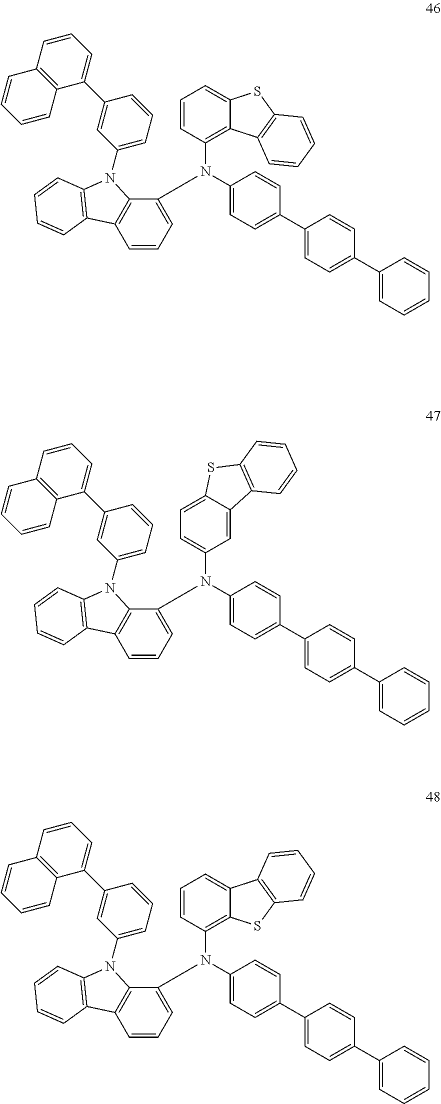 [SMILES: c1ccc(-c2ccc(-c3ccc(N(c4cccc5sc6ccccc6c45)c4cccc5c6ccccc6n(-c6cccc(-c7cccc8ccccc78)c6)c45)cc3)cc2)cc1 ; c1ccc(-c2ccc(-c3ccc(N(c4ccc5sc6ccccc6c5c4)c4cccc5c6ccccc6n(-c6cccc(-c7cccc8ccccc78)c6)c45)cc3)cc2)cc1 ; c1ccc(-c2ccc(-c3ccc(N(c4cccc5c4sc4ccccc45)c4cccc5c6ccccc6n(-c6cccc(-c7cccc8ccccc78)c6)c45)cc3)cc2)cc1] 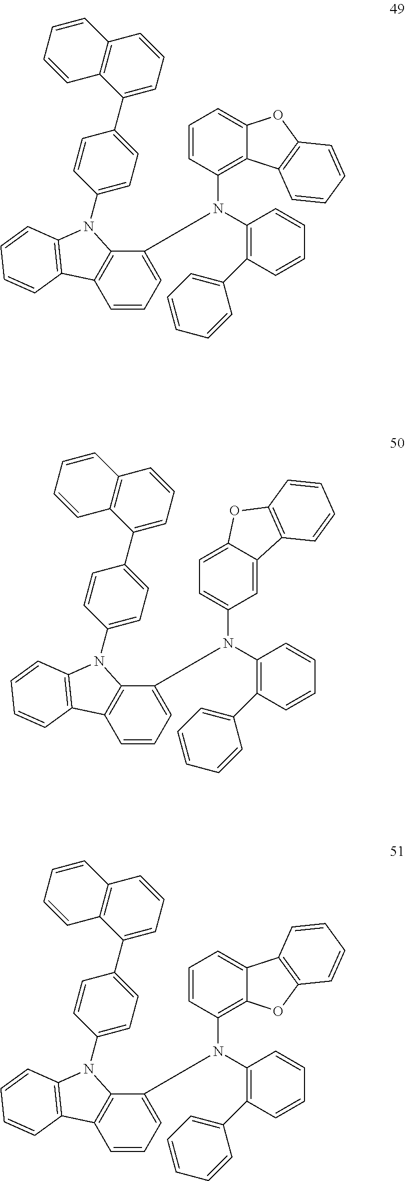 [SMILES: c1ccc(-c2ccccc2N(c2cccc3oc4ccccc4c23)c2cccc3c4ccccc4n(-c4ccc(-c5cccc6ccccc56)cc4)c23)cc1 ; c1ccc(-c2ccccc2N(c2ccc3oc4ccccc4c3c2)c2cccc3c4ccccc4n(-c4ccc(-c5cccc6ccccc56)cc4)c23)cc1 ; c1ccc(-c2ccccc2N(c2cccc3c2oc2ccccc23)c2cccc3c4ccccc4n(-c4ccc(-c5cccc6ccccc56)cc4)c23)cc1] 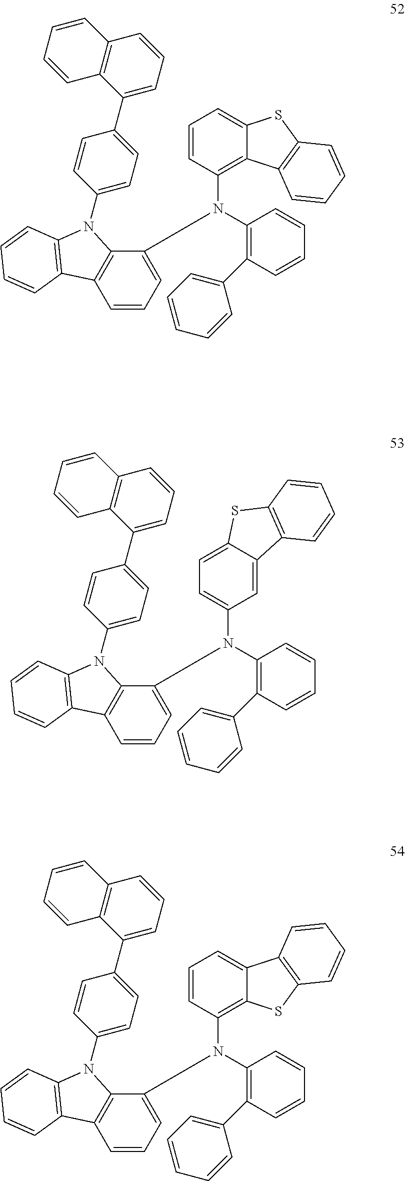 [SMILES: c1ccc(-c2ccccc2N(c2cccc3sc4ccccc4c23)c2cccc3c4ccccc4n(-c4ccc(-c5cccc6ccccc56)cc4)c23)cc1 ; c1ccc(-c2ccccc2N(c2ccc3sc4ccccc4c3c2)c2cccc3c4ccccc4n(-c4ccc(-c5cccc6ccccc56)cc4)c23)cc1 ; c1ccc(-c2ccccc2N(c2cccc3c2sc2ccccc23)c2cccc3c4ccccc4n(-c4ccc(-c5cccc6ccccc56)cc4)c23)cc1] 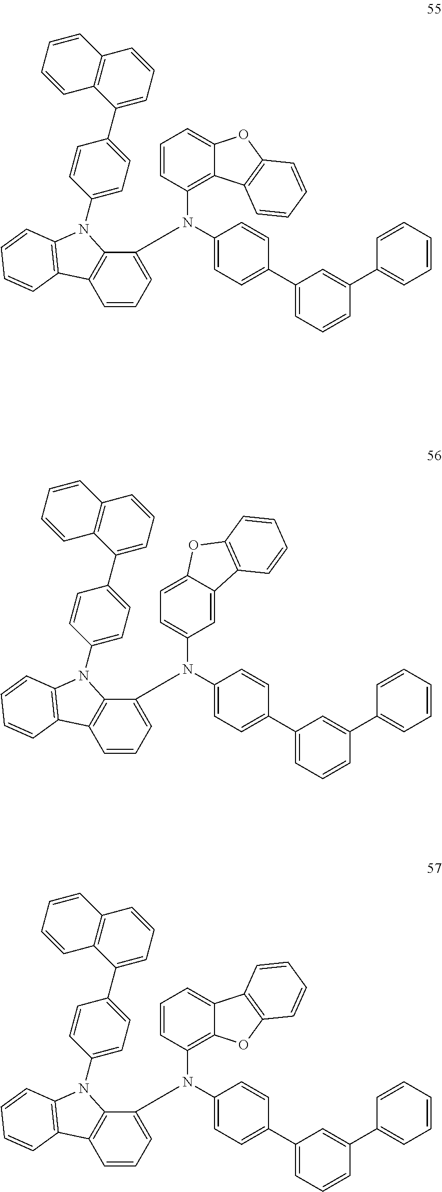 [SMILES: c1ccc(-c2cccc(-c3ccc(N(c4cccc5oc6ccccc6c45)c4cccc5c6ccccc6n(-c6ccc(-c7cccc8ccccc78)cc6)c45)cc3)c2)cc1 ; c1ccc(-c2cccc(-c3ccc(N(c4ccc5oc6ccccc6c5c4)c4cccc5c6ccccc6n(-c6ccc(-c7cccc8ccccc78)cc6)c45)cc3)c2)cc1 ; c1ccc(-c2cccc(-c3ccc(N(c4cccc5c4oc4ccccc45)c4cccc5c6ccccc6n(-c6ccc(-c7cccc8ccccc78)cc6)c45)cc3)c2)cc1] 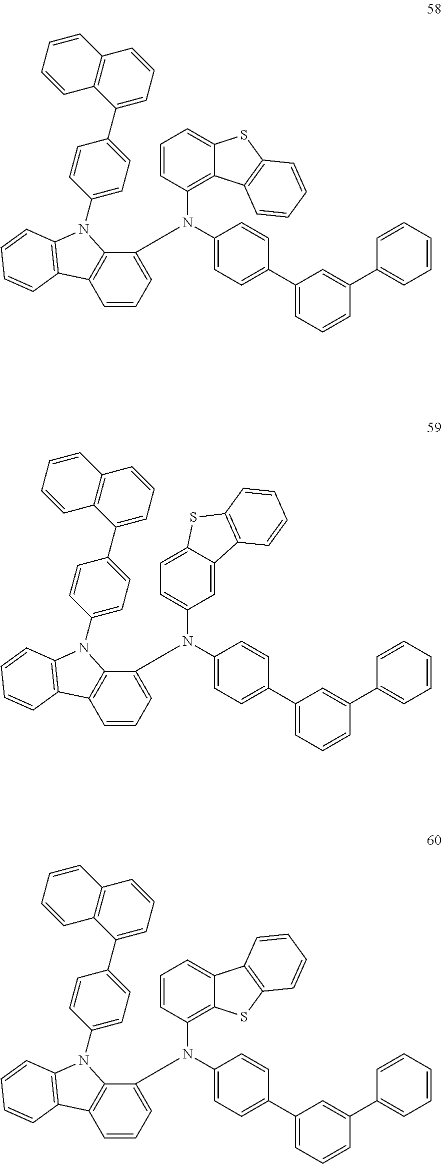 [SMILES: c1ccc(-c2cccc(-c3ccc(N(c4cccc5sc6ccccc6c45)c4cccc5c6ccccc6n(-c6ccc(-c7cccc8ccccc78)cc6)c45)cc3)c2)cc1 ; c1ccc(-c2cccc(-c3ccc(N(c4ccc5sc6ccccc6c5c4)c4cccc5c6ccccc6n(-c6ccc(-c7cccc8ccccc78)cc6)c45)cc3)c2)cc1 ; c1ccc(-c2cccc(-c3ccc(N(c4cccc5c4sc4ccccc45)c4cccc5c6ccccc6n(-c6ccc(-c7cccc8ccccc78)cc6)c45)cc3)c2)cc1] 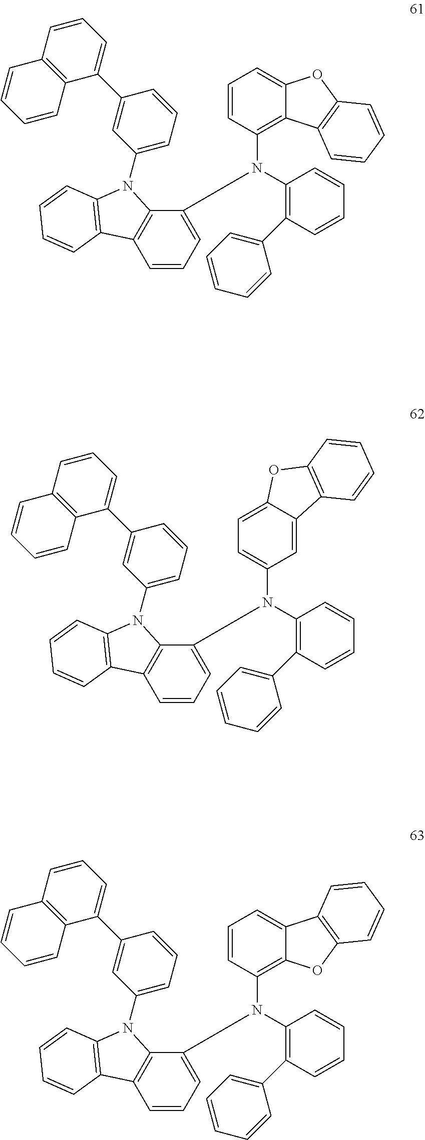 [SMILES: c1ccc(-c2ccccc2N(c2cccc3oc4ccccc4c23)c2cccc3c4ccccc4n(-c4cccc(-c5cccc6ccccc56)c4)c23)cc1 ; c1ccc(-c2ccccc2N(c2ccc3oc4ccccc4c3c2)c2cccc3c4ccccc4n(-c4cccc(-c5cccc6ccccc56)c4)c23)cc1 ; c1ccc(-c2ccccc2N(c2cccc3c2oc2ccccc23)c2cccc3c4ccccc4n(-c4cccc(-c5cccc6ccccc56)c4)c23)cc1] 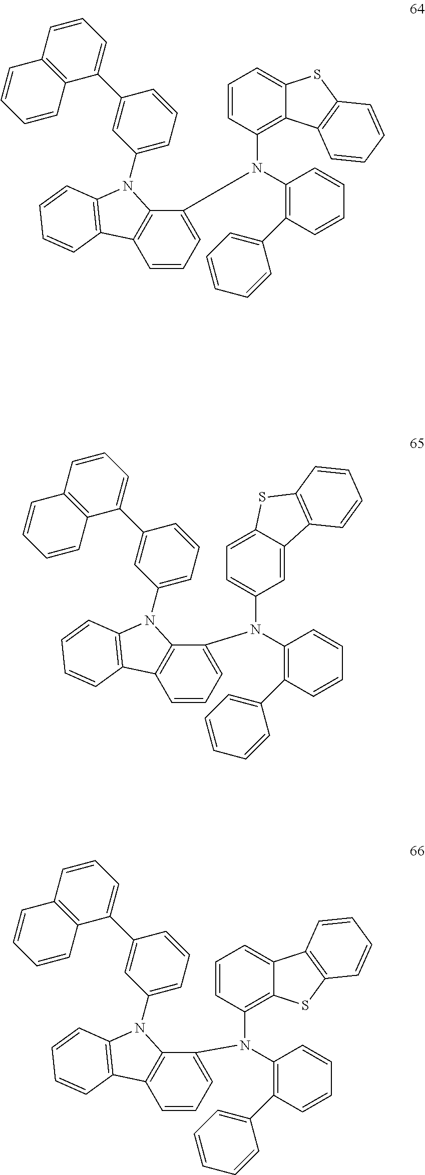 [SMILES: c1ccc(-c2ccccc2N(c2cccc3sc4ccccc4c23)c2cccc3c4ccccc4n(-c4cccc(-c5cccc6ccccc56)c4)c23)cc1 ; c1ccc(-c2ccccc2N(c2ccc3sc4ccccc4c3c2)c2cccc3c4ccccc4n(-c4cccc(-c5cccc6ccccc56)c4)c23)cc1 ; c1ccc(-c2ccccc2N(c2cccc3c2sc2ccccc23)c2cccc3c4ccccc4n(-c4cccc(-c5cccc6ccccc56)c4)c23)cc1] 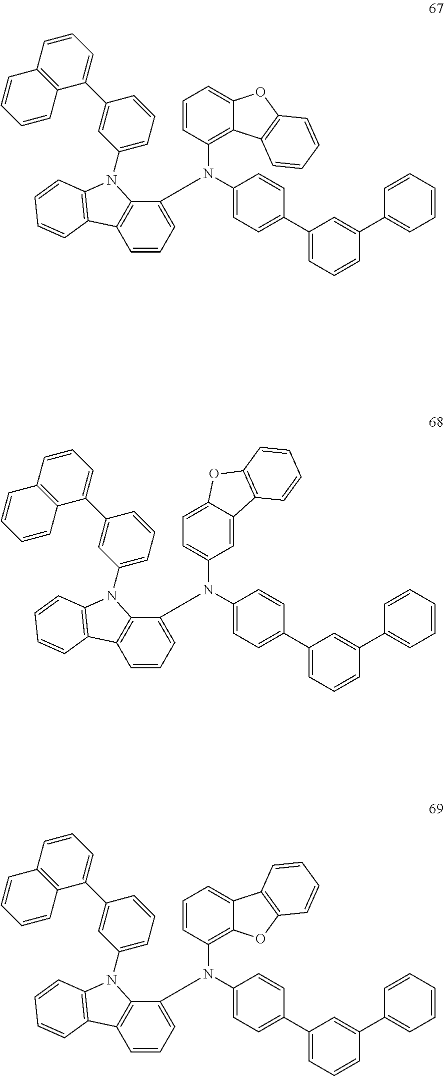 [SMILES: c1ccc(-c2cccc(-c3ccc(N(c4cccc5oc6ccccc6c45)c4cccc5c6ccccc6n(-c6cccc(-c7cccc8ccccc78)c6)c45)cc3)c2)cc1 ; c1ccc(-c2cccc(-c3ccc(N(c4ccc5oc6ccccc6c5c4)c4cccc5c6ccccc6n(-c6cccc(-c7cccc8ccccc78)c6)c45)cc3)c2)cc1 ; c1ccc(-c2cccc(-c3ccc(N(c4cccc5c4oc4ccccc45)c4cccc5c6ccccc6n(-c6cccc(-c7cccc8ccccc78)c6)c45)cc3)c2)cc1] 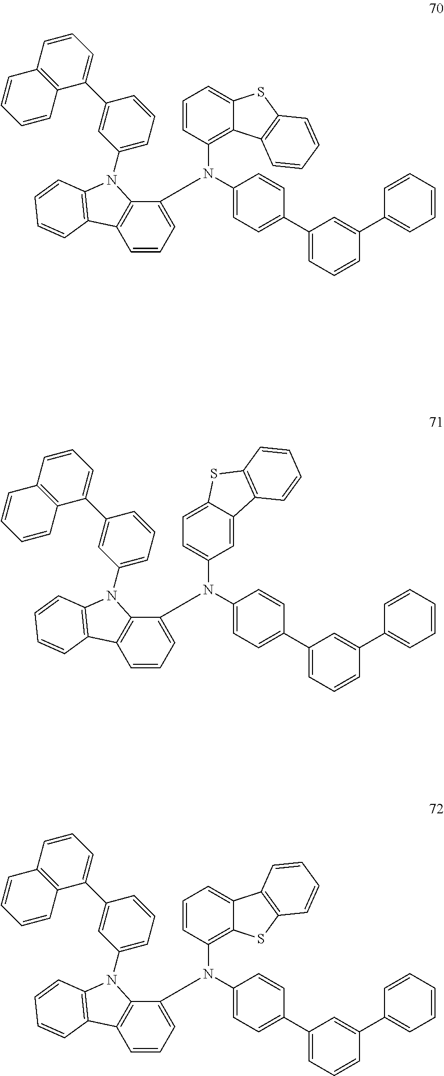 [SMILES: c1ccc(-c2cccc(-c3ccc(N(c4cccc5sc6ccccc6c45)c4cccc5c6ccccc6n(-c6cccc(-c7cccc8ccccc78)c6)c45)cc3)c2)cc1 ; c1ccc(-c2cccc(-c3ccc(N(c4ccc5sc6ccccc6c5c4)c4cccc5c6ccccc6n(-c6cccc(-c7cccc8ccccc78)c6)c45)cc3)c2)cc1 ; c1ccc(-c2cccc(-c3ccc(N(c4cccc5c4sc4ccccc45)c4cccc5c6ccccc6n(-c6cccc(-c7cccc8ccccc78)c6)c45)cc3)c2)cc1] 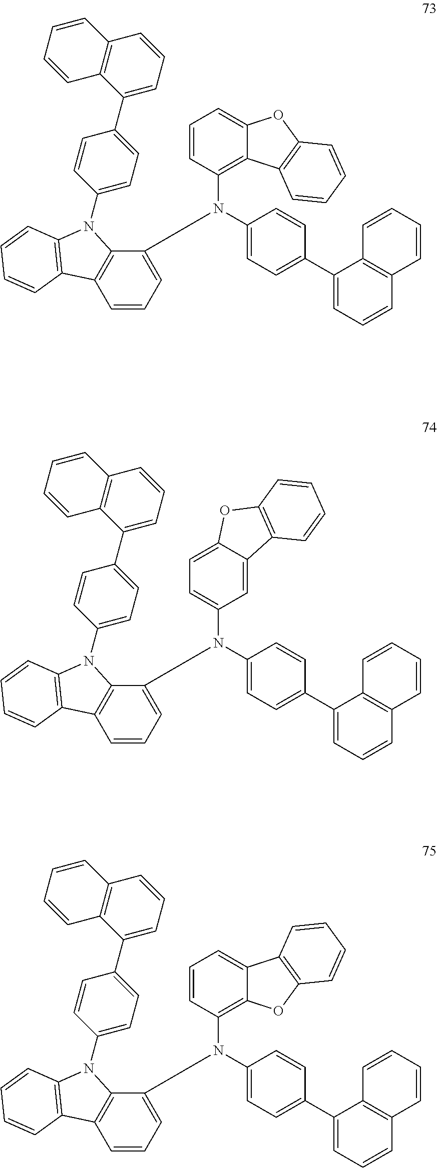 [SMILES: c1ccc2c(-c3ccc(N(c4cccc5oc6ccccc6c45)c4cccc5c6ccccc6n(-c6ccc(-c7cccc8ccccc78)cc6)c45)cc3)cccc2c1 ; c1ccc2c(-c3ccc(N(c4ccc5oc6ccccc6c5c4)c4cccc5c6ccccc6n(-c6ccc(-c7cccc8ccccc78)cc6)c45)cc3)cccc2c1 ; c1ccc2c(-c3ccc(N(c4cccc5c4oc4ccccc45)c4cccc5c6ccccc6n(-c6ccc(-c7cccc8ccccc78)cc6)c45)cc3)cccc2c1] 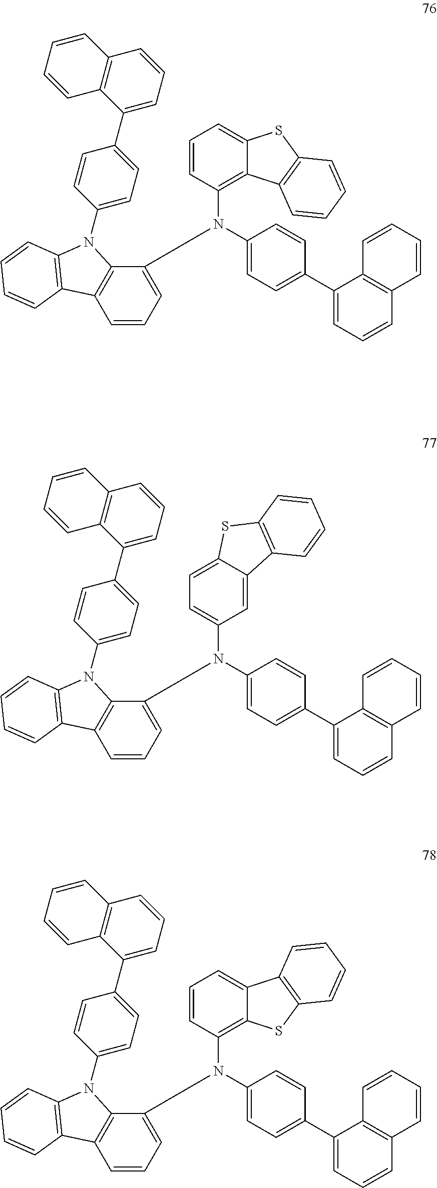 [SMILES: c1ccc2c(-c3ccc(N(c4cccc5sc6ccccc6c45)c4cccc5c6ccccc6n(-c6ccc(-c7cccc8ccccc78)cc6)c45)cc3)cccc2c1 ; c1ccc2c(-c3ccc(N(c4ccc5sc6ccccc6c5c4)c4cccc5c6ccccc6n(-c6ccc(-c7cccc8ccccc78)cc6)c45)cc3)cccc2c1 ; c1ccc2c(-c3ccc(N(c4cccc5c4sc4ccccc45)c4cccc5c6ccccc6n(-c6ccc(-c7cccc8ccccc78)cc6)c45)cc3)cccc2c1] 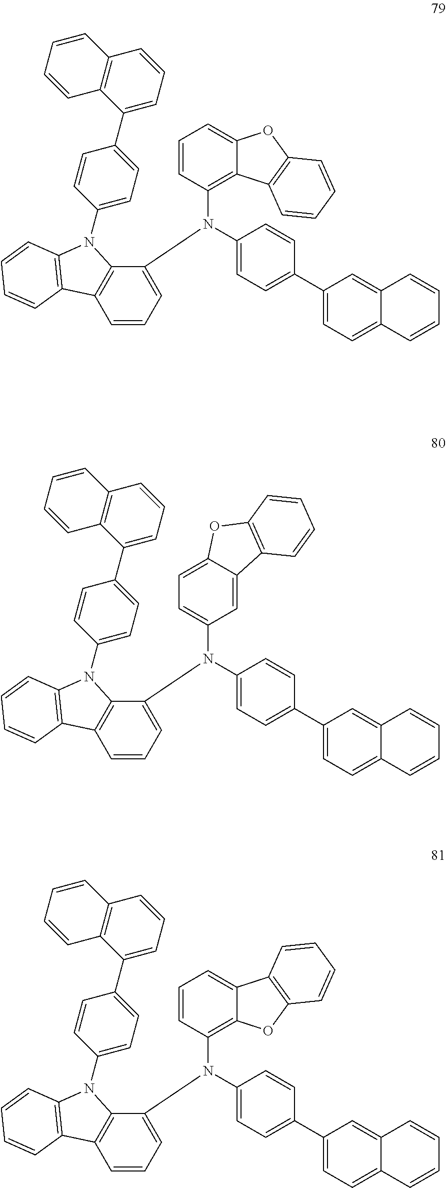 [SMILES: c1ccc2cc(-c3ccc(N(c4cccc5oc6ccccc6c45)c4cccc5c6ccccc6n(-c6ccc(-c7cccc8ccccc78)cc6)c45)cc3)ccc2c1 ; c1ccc2cc(-c3ccc(N(c4ccc5oc6ccccc6c5c4)c4cccc5c6ccccc6n(-c6ccc(-c7cccc8ccccc78)cc6)c45)cc3)ccc2c1 ; c1ccc2cc(-c3ccc(N(c4cccc5c4oc4ccccc45)c4cccc5c6ccccc6n(-c6ccc(-c7cccc8ccccc78)cc6)c45)cc3)ccc2c1] 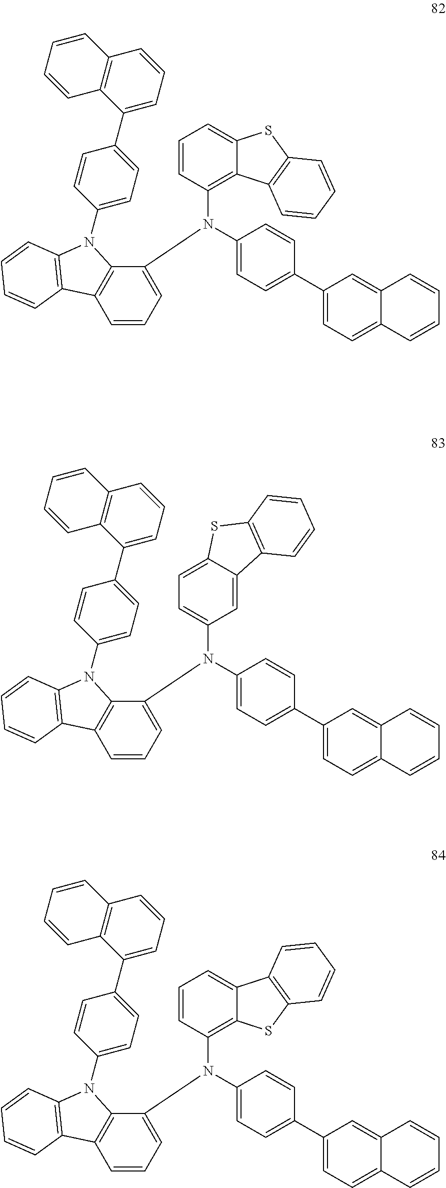 [SMILES: c1ccc2cc(-c3ccc(N(c4cccc5sc6ccccc6c45)c4cccc5c6ccccc6n(-c6ccc(-c7cccc8ccccc78)cc6)c45)cc3)ccc2c1 ; c1ccc2cc(-c3ccc(N(c4ccc5sc6ccccc6c5c4)c4cccc5c6ccccc6n(-c6ccc(-c7cccc8ccccc78)cc6)c45)cc3)ccc2c1 ; c1ccc2cc(-c3ccc(N(c4cccc5c4sc4ccccc45)c4cccc5c6ccccc6n(-c6ccc(-c7cccc8ccccc78)cc6)c45)cc3)ccc2c1] 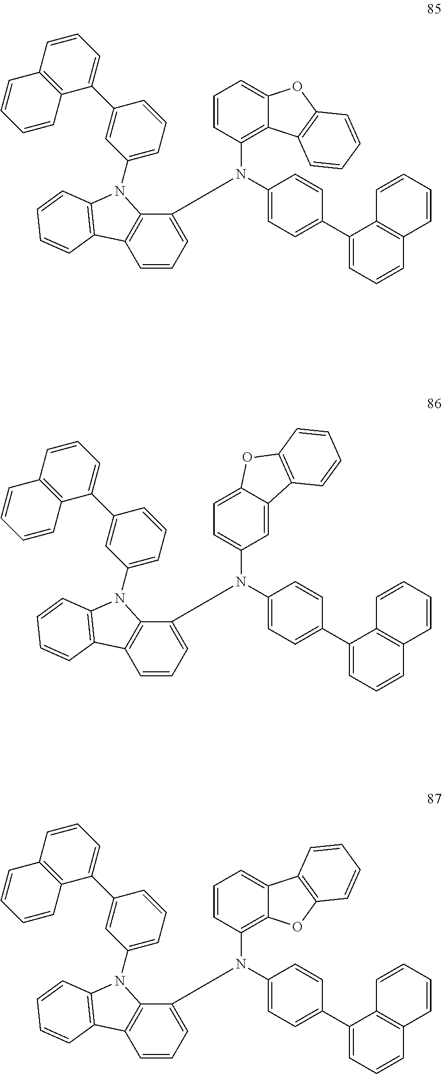 [SMILES: c1cc(-c2cccc3ccccc23)cc(-n2c3ccccc3c3cccc(N(c4ccc(-c5cccc6ccccc56)cc4)c4cccc5oc6ccccc6c45)c32)c1 ; c1cc(-c2cccc3ccccc23)cc(-n2c3ccccc3c3cccc(N(c4ccc(-c5cccc6ccccc56)cc4)c4ccc5oc6ccccc6c5c4)c32)c1 ; c1cc(-c2cccc3ccccc23)cc(-n2c3ccccc3c3cccc(N(c4ccc(-c5cccc6ccccc56)cc4)c4cccc5c4oc4ccccc45)c32)c1] 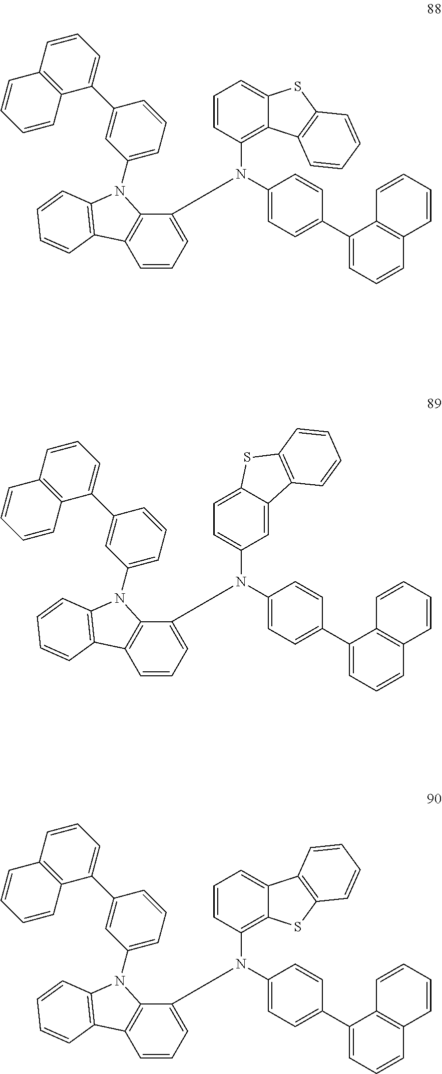 [SMILES: c1cc(-c2cccc3ccccc23)cc(-n2c3ccccc3c3cccc(N(c4ccc(-c5cccc6ccccc56)cc4)c4cccc5sc6ccccc6c45)c32)c1 ; c1cc(-c2cccc3ccccc23)cc(-n2c3ccccc3c3cccc(N(c4ccc(-c5cccc6ccccc56)cc4)c4ccc5sc6ccccc6c5c4)c32)c1 ; c1cc(-c2cccc3ccccc23)cc(-n2c3ccccc3c3cccc(N(c4ccc(-c5cccc6ccccc56)cc4)c4cccc5c4sc4ccccc45)c32)c1] 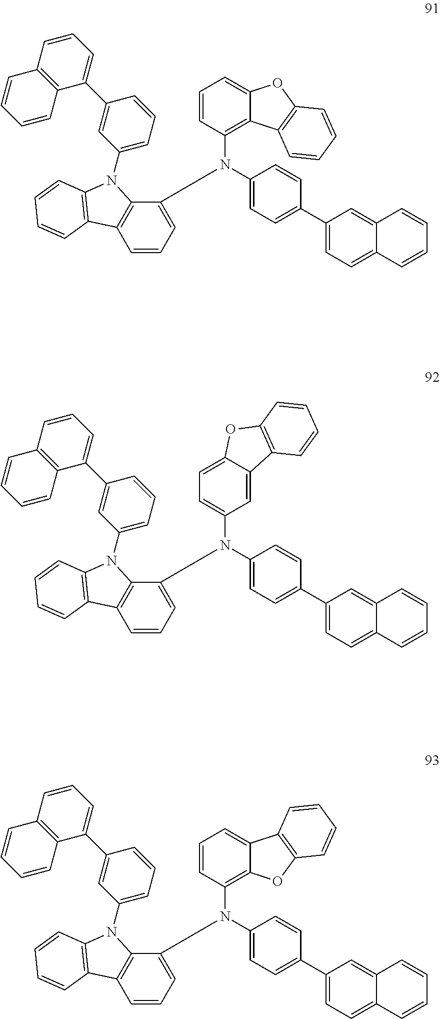 [SMILES: c1cc(-c2cccc3ccccc23)cc(-n2c3ccccc3c3cccc(N(c4ccc(-c5ccc6ccccc6c5)cc4)c4cccc5oc6ccccc6c45)c32)c1 ; c1cc(-c2cccc3ccccc23)cc(-n2c3ccccc3c3cccc(N(c4ccc(-c5ccc6ccccc6c5)cc4)c4ccc5oc6ccccc6c5c4)c32)c1 ; c1cc(-c2cccc3ccccc23)cc(-n2c3ccccc3c3cccc(N(c4ccc(-c5ccc6ccccc6c5)cc4)c4cccc5c4oc4ccccc45)c32)c1] 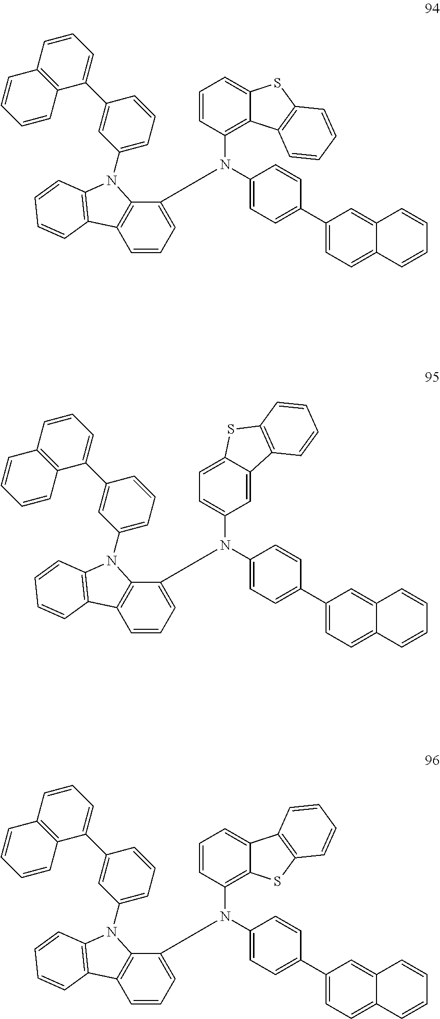 [SMILES: c1cc(-c2cccc3ccccc23)cc(-n2c3ccccc3c3cccc(N(c4ccc(-c5ccc6ccccc6c5)cc4)c4cccc5sc6ccccc6c45)c32)c1 ; c1cc(-c2cccc3ccccc23)cc(-n2c3ccccc3c3cccc(N(c4ccc(-c5ccc6ccccc6c5)cc4)c4ccc5sc6ccccc6c5c4)c32)c1 ; c1cc(-c2cccc3ccccc23)cc(-n2c3ccccc3c3cccc(N(c4ccc(-c5ccc6ccccc6c5)cc4)c4cccc5c4sc4ccccc45)c32)c1] 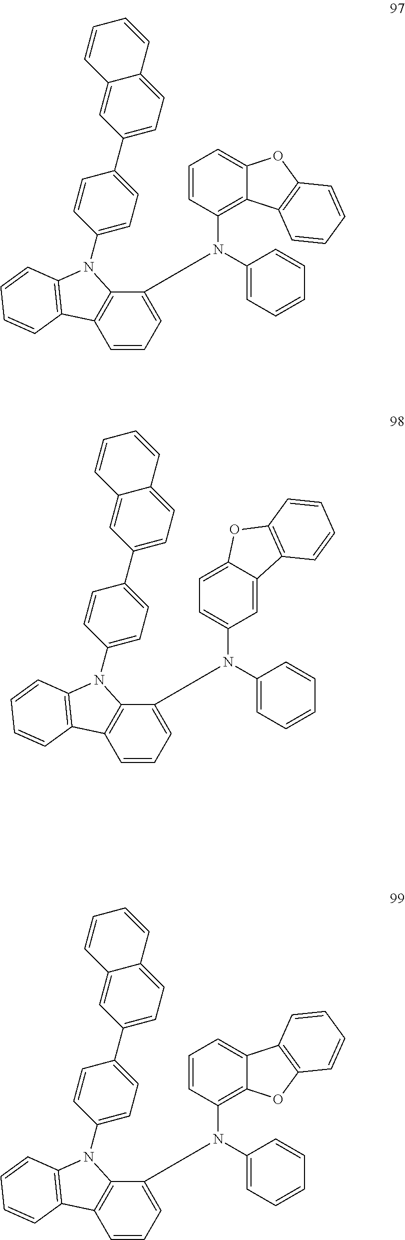 [SMILES: c1ccc(N(c2cccc3oc4ccccc4c23)c2cccc3c4ccccc4n(-c4ccc(-c5ccc6ccccc6c5)cc4)c23)cc1 ; c1ccc(N(c2ccc3oc4ccccc4c3c2)c2cccc3c4ccccc4n(-c4ccc(-c5ccc6ccccc6c5)cc4)c23)cc1 ; c1ccc(N(c2cccc3c2oc2ccccc23)c2cccc3c4ccccc4n(-c4ccc(-c5ccc6ccccc6c5)cc4)c23)cc1] 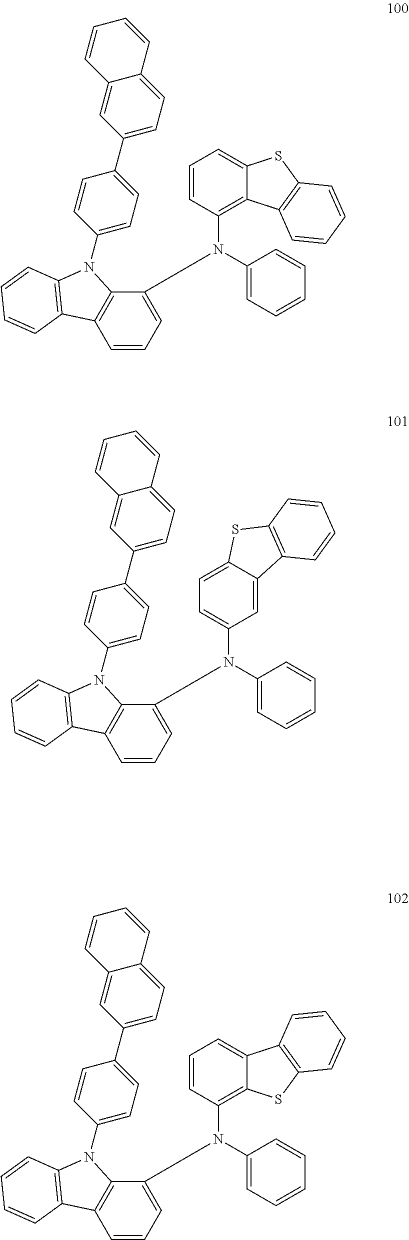 [SMILES: c1ccc(N(c2cccc3sc4ccccc4c23)c2cccc3c4ccccc4n(-c4ccc(-c5ccc6ccccc6c5)cc4)c23)cc1 ; c1ccc(N(c2ccc3sc4ccccc4c3c2)c2cccc3c4ccccc4n(-c4ccc(-c5ccc6ccccc6c5)cc4)c23)cc1 ; c1ccc(N(c2cccc3c2sc2ccccc23)c2cccc3c4ccccc4n(-c4ccc(-c5ccc6ccccc6c5)cc4)c23)cc1] 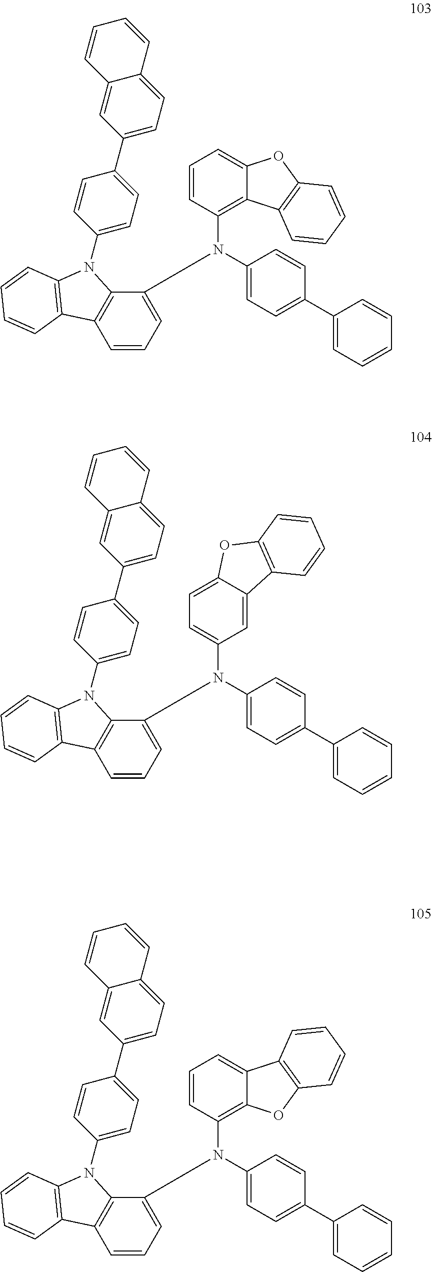 [SMILES: c1ccc(-c2ccc(N(c3cccc4oc5ccccc5c34)c3cccc4c5ccccc5n(-c5ccc(-c6ccc7ccccc7c6)cc5)c34)cc2)cc1 ; c1ccc(-c2ccc(N(c3ccc4oc5ccccc5c4c3)c3cccc4c5ccccc5n(-c5ccc(-c6ccc7ccccc7c6)cc5)c34)cc2)cc1 ; c1ccc(-c2ccc(N(c3cccc4c3oc3ccccc34)c3cccc4c5ccccc5n(-c5ccc(-c6ccc7ccccc7c6)cc5)c34)cc2)cc1] 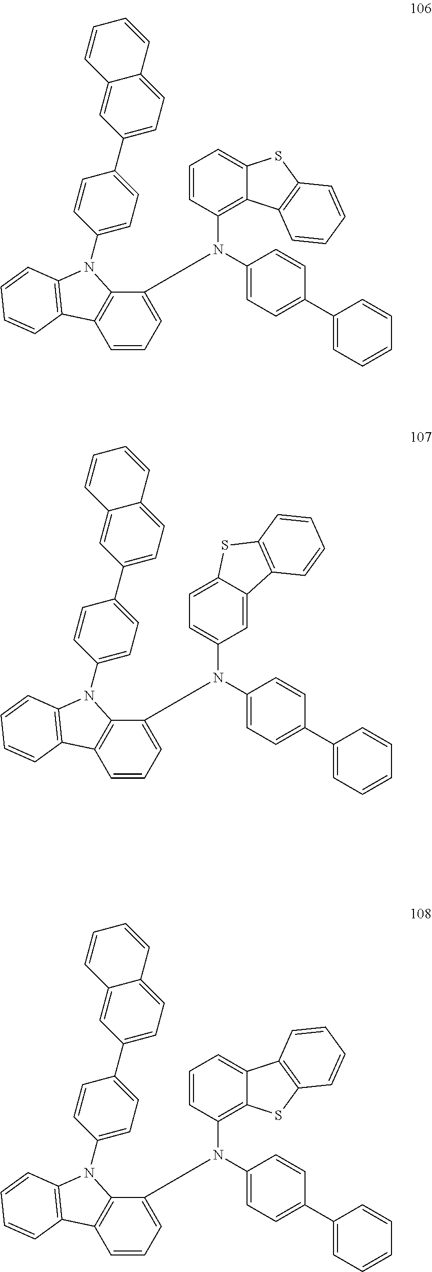 [SMILES: c1ccc(-c2ccc(N(c3cccc4sc5ccccc5c34)c3cccc4c5ccccc5n(-c5ccc(-c6ccc7ccccc7c6)cc5)c34)cc2)cc1 ; c1ccc(-c2ccc(N(c3ccc4sc5ccccc5c4c3)c3cccc4c5ccccc5n(-c5ccc(-c6ccc7ccccc7c6)cc5)c34)cc2)cc1 ; c1ccc(-c2ccc(N(c3cccc4c3sc3ccccc34)c3cccc4c5ccccc5n(-c5ccc(-c6ccc7ccccc7c6)cc5)c34)cc2)cc1] 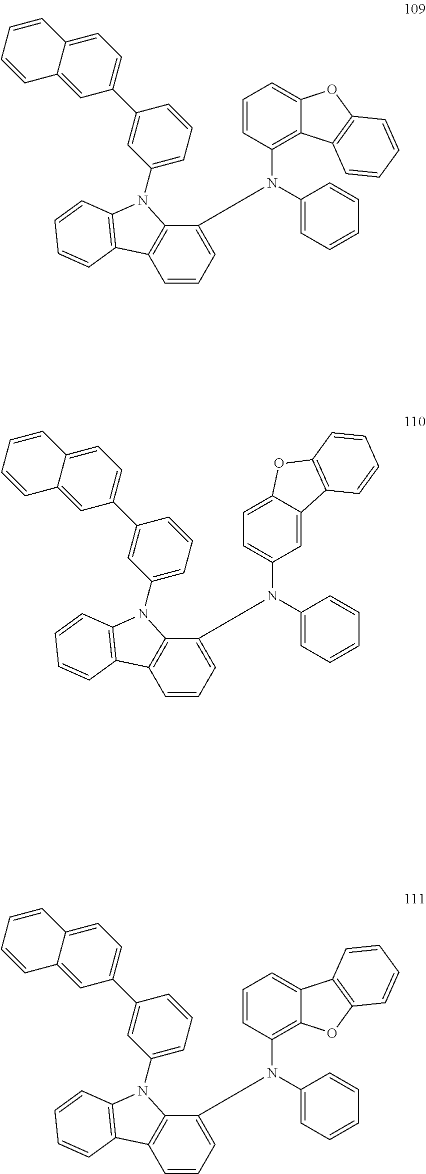 [SMILES: c1ccc(N(c2cccc3oc4ccccc4c23)c2cccc3c4ccccc4n(-c4cccc(-c5ccc6ccccc6c5)c4)c23)cc1 ; c1ccc(N(c2ccc3oc4ccccc4c3c2)c2cccc3c4ccccc4n(-c4cccc(-c5ccc6ccccc6c5)c4)c23)cc1 ; c1ccc(N(c2cccc3c2oc2ccccc23)c2cccc3c4ccccc4n(-c4cccc(-c5ccc6ccccc6c5)c4)c23)cc1] 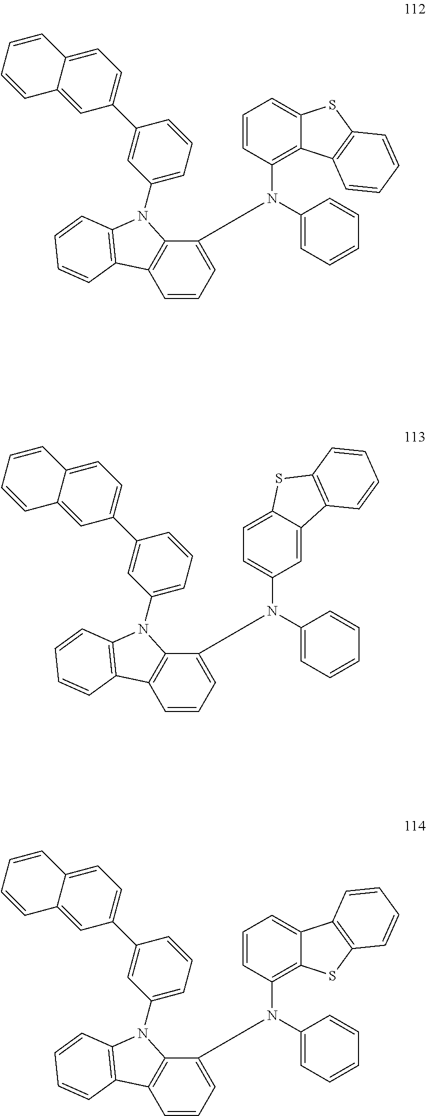 [SMILES: c1ccc(N(c2cccc3sc4ccccc4c23)c2cccc3c4ccccc4n(-c4cccc(-c5ccc6ccccc6c5)c4)c23)cc1 ; c1ccc(N(c2ccc3sc4ccccc4c3c2)c2cccc3c4ccccc4n(-c4cccc(-c5ccc6ccccc6c5)c4)c23)cc1 ; c1ccc(N(c2cccc3c2sc2ccccc23)c2cccc3c4ccccc4n(-c4cccc(-c5ccc6ccccc6c5)c4)c23)cc1] 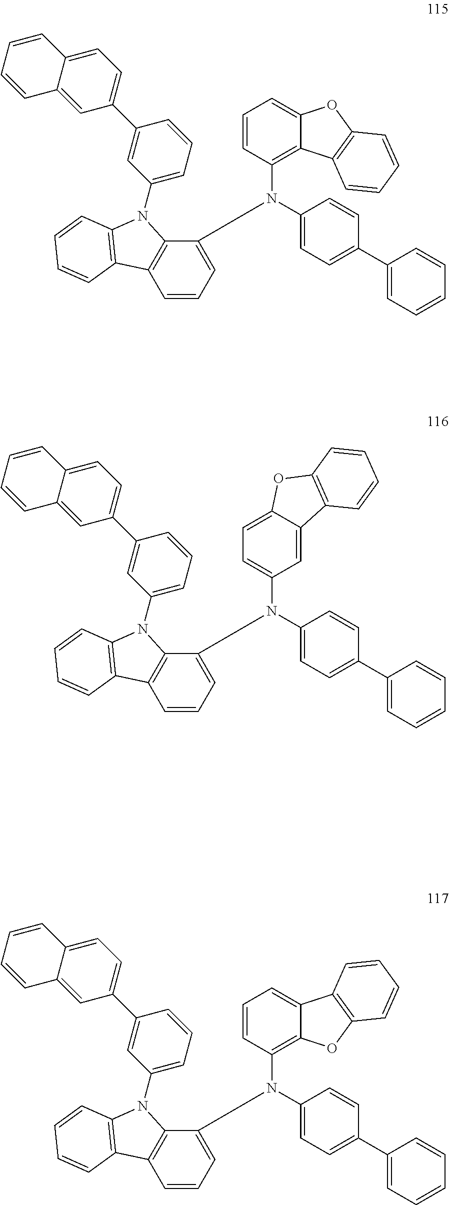 [SMILES: c1ccc(-c2ccc(N(c3cccc4oc5ccccc5c34)c3cccc4c5ccccc5n(-c5cccc(-c6ccc7ccccc7c6)c5)c34)cc2)cc1 ; c1ccc(-c2ccc(N(c3ccc4oc5ccccc5c4c3)c3cccc4c5ccccc5n(-c5cccc(-c6ccc7ccccc7c6)c5)c34)cc2)cc1 ; c1ccc(-c2ccc(N(c3cccc4c3oc3ccccc34)c3cccc4c5ccccc5n(-c5cccc(-c6ccc7ccccc7c6)c5)c34)cc2)cc1] 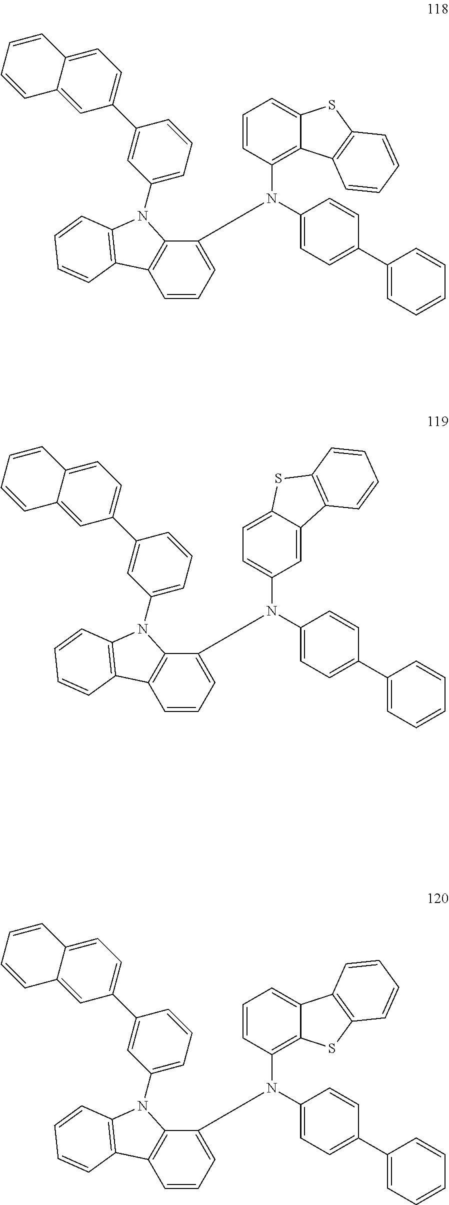 [SMILES: c1ccc(-c2ccc(N(c3cccc4sc5ccccc5c34)c3cccc4c5ccccc5n(-c5cccc(-c6ccc7ccccc7c6)c5)c34)cc2)cc1 ; c1ccc(-c2ccc(N(c3ccc4sc5ccccc5c4c3)c3cccc4c5ccccc5n(-c5cccc(-c6ccc7ccccc7c6)c5)c34)cc2)cc1 ; c1ccc(-c2ccc(N(c3cccc4c3sc3ccccc34)c3cccc4c5ccccc5n(-c5cccc(-c6ccc7ccccc7c6)c5)c34)cc2)cc1] 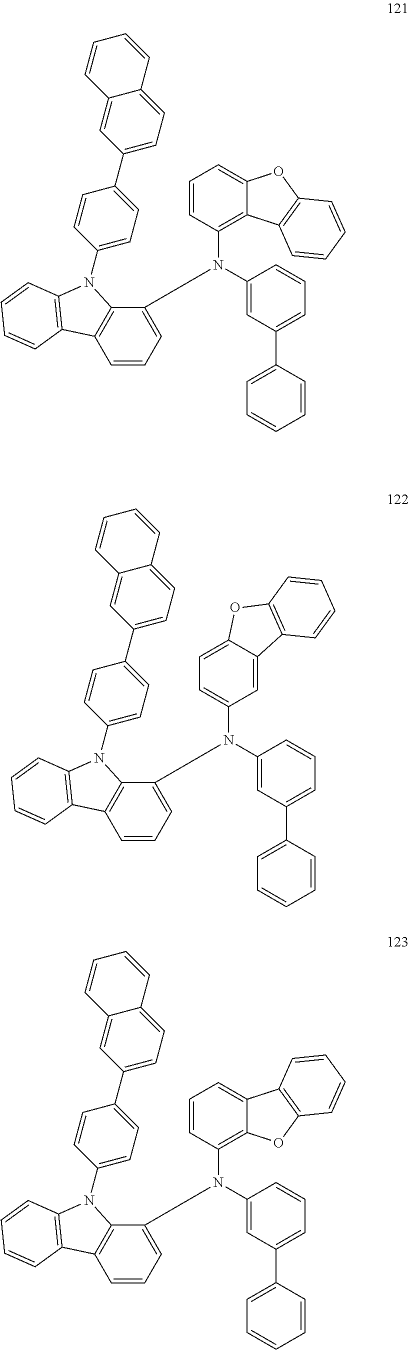 [SMILES: c1ccc(-c2cccc(N(c3cccc4oc5ccccc5c34)c3cccc4c5ccccc5n(-c5ccc(-c6ccc7ccccc7c6)cc5)c34)c2)cc1 ; c1ccc(-c2cccc(N(c3ccc4oc5ccccc5c4c3)c3cccc4c5ccccc5n(-c5ccc(-c6ccc7ccccc7c6)cc5)c34)c2)cc1 ; c1ccc(-c2cccc(N(c3cccc4c3oc3ccccc34)c3cccc4c5ccccc5n(-c5ccc(-c6ccc7ccccc7c6)cc5)c34)c2)cc1] 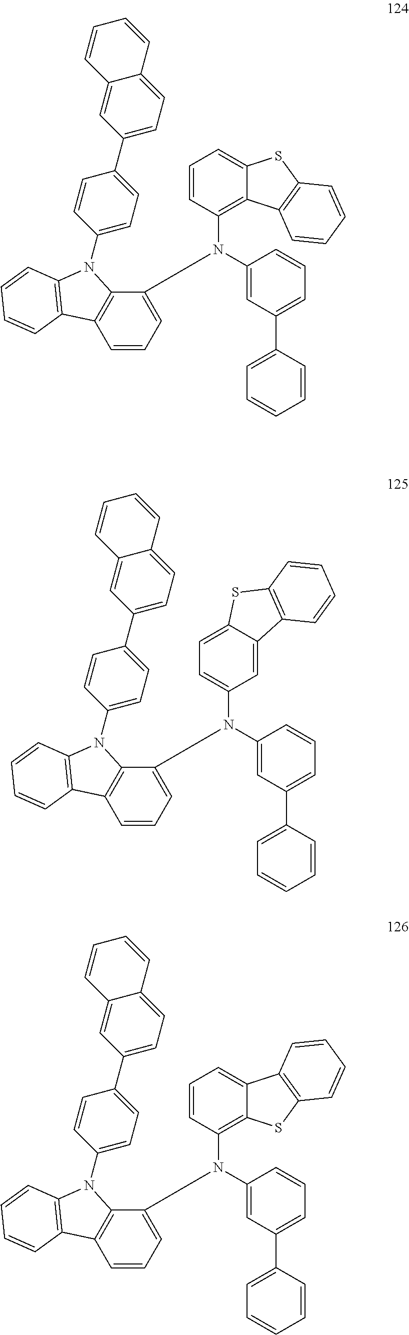 [SMILES: c1ccc(-c2cccc(N(c3cccc4sc5ccccc5c34)c3cccc4c5ccccc5n(-c5ccc(-c6ccc7ccccc7c6)cc5)c34)c2)cc1 ; c1ccc(-c2cccc(N(c3ccc4sc5ccccc5c4c3)c3cccc4c5ccccc5n(-c5ccc(-c6ccc7ccccc7c6)cc5)c34)c2)cc1 ; c1ccc(-c2cccc(N(c3cccc4c3sc3ccccc34)c3cccc4c5ccccc5n(-c5ccc(-c6ccc7ccccc7c6)cc5)c34)c2)cc1] 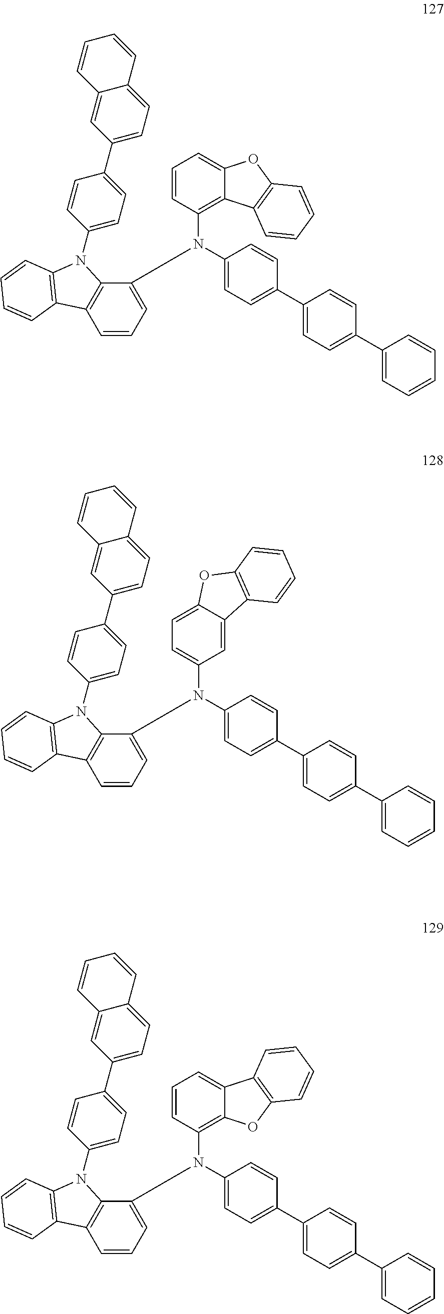 [SMILES: c1ccc(-c2ccc(-c3ccc(N(c4cccc5oc6ccccc6c45)c4cccc5c6ccccc6n(-c6ccc(-c7ccc8ccccc8c7)cc6)c45)cc3)cc2)cc1 ; c1ccc(-c2ccc(-c3ccc(N(c4ccc5oc6ccccc6c5c4)c4cccc5c6ccccc6n(-c6ccc(-c7ccc8ccccc8c7)cc6)c45)cc3)cc2)cc1 ; c1ccc(-c2ccc(-c3ccc(N(c4cccc5c4oc4ccccc45)c4cccc5c6ccccc6n(-c6ccc(-c7ccc8ccccc8c7)cc6)c45)cc3)cc2)cc1] 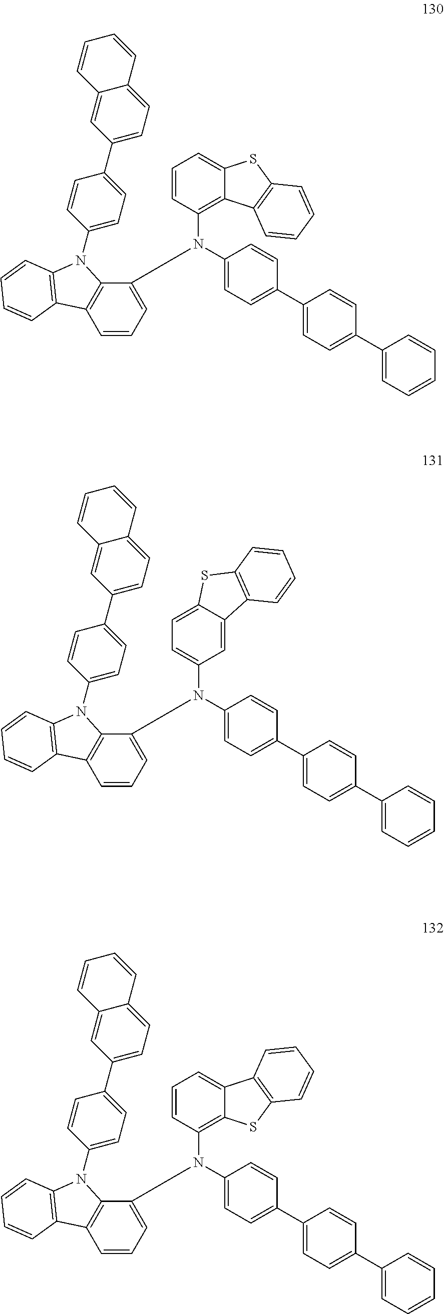 [SMILES: c1ccc(-c2ccc(-c3ccc(N(c4cccc5sc6ccccc6c45)c4cccc5c6ccccc6n(-c6ccc(-c7ccc8ccccc8c7)cc6)c45)cc3)cc2)cc1 ; c1ccc(-c2ccc(-c3ccc(N(c4ccc5sc6ccccc6c5c4)c4cccc5c6ccccc6n(-c6ccc(-c7ccc8ccccc8c7)cc6)c45)cc3)cc2)cc1 ; c1ccc(-c2ccc(-c3ccc(N(c4cccc5c4sc4ccccc45)c4cccc5c6ccccc6n(-c6ccc(-c7ccc8ccccc8c7)cc6)c45)cc3)cc2)cc1] 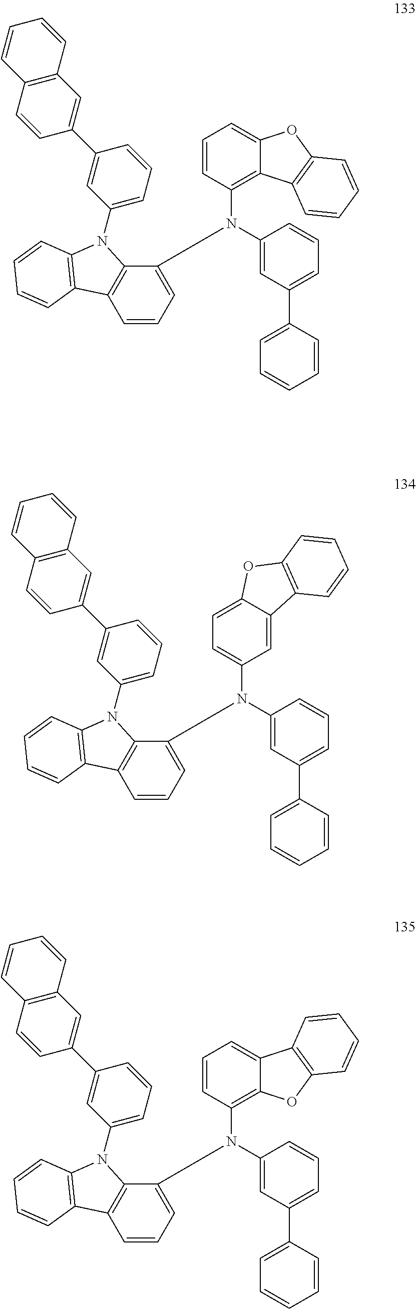 [SMILES: c1ccc(-c2cccc(N(c3cccc4oc5ccccc5c34)c3cccc4c5ccccc5n(-c5cccc(-c6ccc7ccccc7c6)c5)c34)c2)cc1 ; c1ccc(-c2cccc(N(c3ccc4oc5ccccc5c4c3)c3cccc4c5ccccc5n(-c5cccc(-c6ccc7ccccc7c6)c5)c34)c2)cc1 ; c1ccc(-c2cccc(N(c3cccc4c3oc3ccccc34)c3cccc4c5ccccc5n(-c5cccc(-c6ccc7ccccc7c6)c5)c34)c2)cc1] 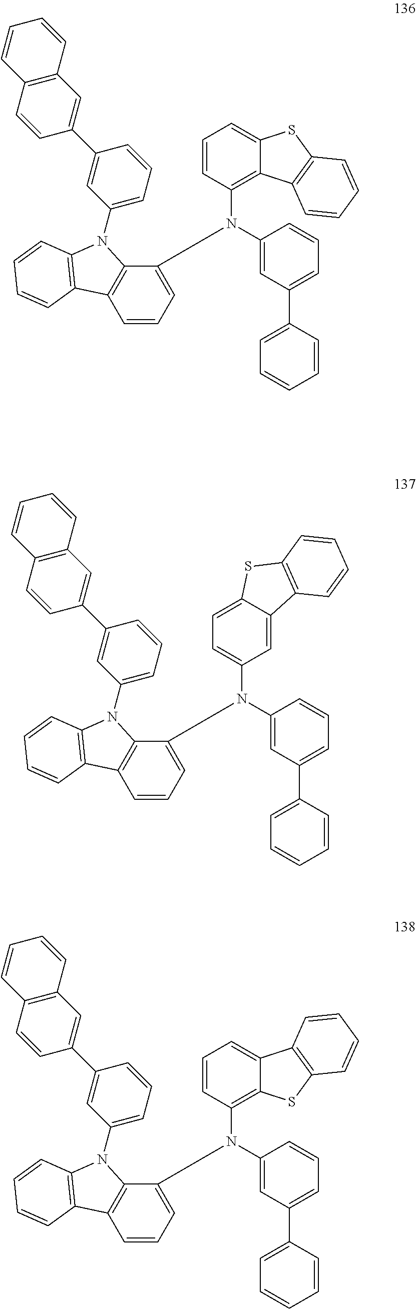 [SMILES: c1ccc(-c2cccc(N(c3cccc4sc5ccccc5c34)c3cccc4c5ccccc5n(-c5cccc(-c6ccc7ccccc7c6)c5)c34)c2)cc1 ; c1ccc(-c2cccc(N(c3ccc4sc5ccccc5c4c3)c3cccc4c5ccccc5n(-c5cccc(-c6ccc7ccccc7c6)c5)c34)c2)cc1 ; c1ccc(-c2cccc(N(c3cccc4c3sc3ccccc34)c3cccc4c5ccccc5n(-c5cccc(-c6ccc7ccccc7c6)c5)c34)c2)cc1] 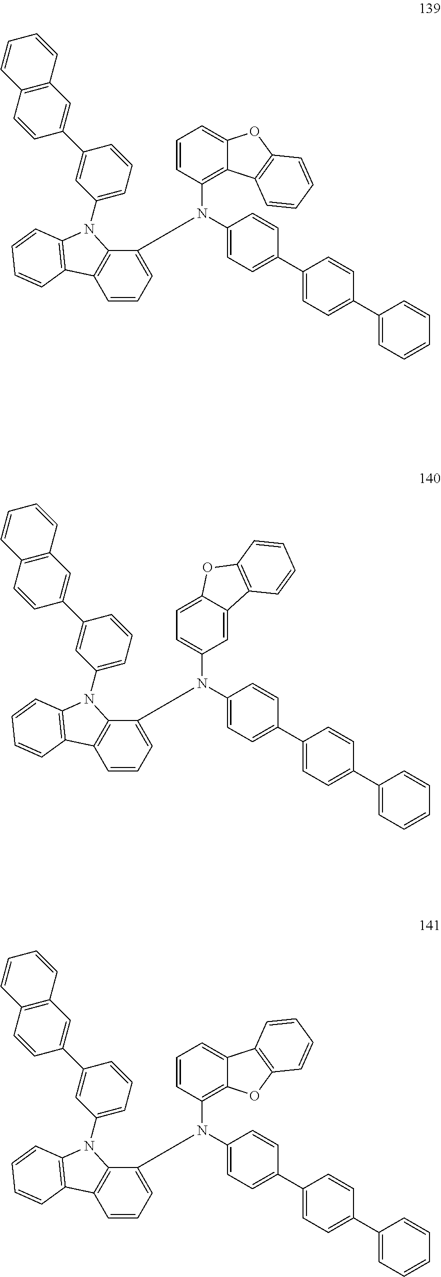 [SMILES: c1ccc(-c2ccc(-c3ccc(N(c4cccc5oc6ccccc6c45)c4cccc5c6ccccc6n(-c6cccc(-c7ccc8ccccc8c7)c6)c45)cc3)cc2)cc1 ; c1ccc(-c2ccc(-c3ccc(N(c4ccc5oc6ccccc6c5c4)c4cccc5c6ccccc6n(-c6cccc(-c7ccc8ccccc8c7)c6)c45)cc3)cc2)cc1 ; c1ccc(-c2ccc(-c3ccc(N(c4cccc5c4oc4ccccc45)c4cccc5c6ccccc6n(-c6cccc(-c7ccc8ccccc8c7)c6)c45)cc3)cc2)cc1] 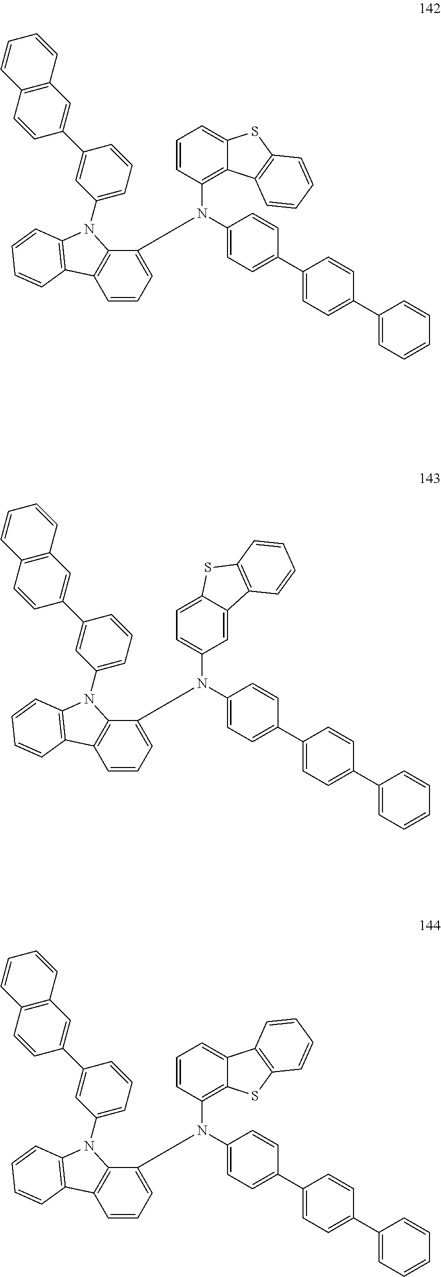 [SMILES: c1ccc(-c2ccc(-c3ccc(N(c4cccc5sc6ccccc6c45)c4cccc5c6ccccc6n(-c6cccc(-c7ccc8ccccc8c7)c6)c45)cc3)cc2)cc1 ; c1ccc(-c2ccc(-c3ccc(N(c4ccc5sc6ccccc6c5c4)c4cccc5c6ccccc6n(-c6cccc(-c7ccc8ccccc8c7)c6)c45)cc3)cc2)cc1 ; c1ccc(-c2ccc(-c3ccc(N(c4cccc5c4sc4ccccc45)c4cccc5c6ccccc6n(-c6cccc(-c7ccc8ccccc8c7)c6)c45)cc3)cc2)cc1] 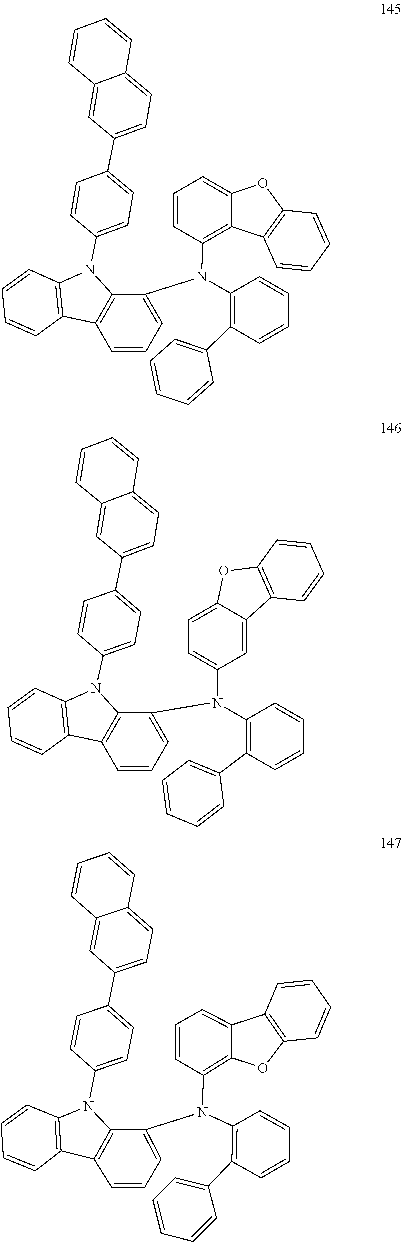 [SMILES: c1ccc(-c2ccccc2N(c2cccc3oc4ccccc4c23)c2cccc3c4ccccc4n(-c4ccc(-c5ccc6ccccc6c5)cc4)c23)cc1 ; c1ccc(-c2ccccc2N(c2ccc3oc4ccccc4c3c2)c2cccc3c4ccccc4n(-c4ccc(-c5ccc6ccccc6c5)cc4)c23)cc1 ; c1ccc(-c2ccccc2N(c2cccc3c2oc2ccccc23)c2cccc3c4ccccc4n(-c4ccc(-c5ccc6ccccc6c5)cc4)c23)cc1] 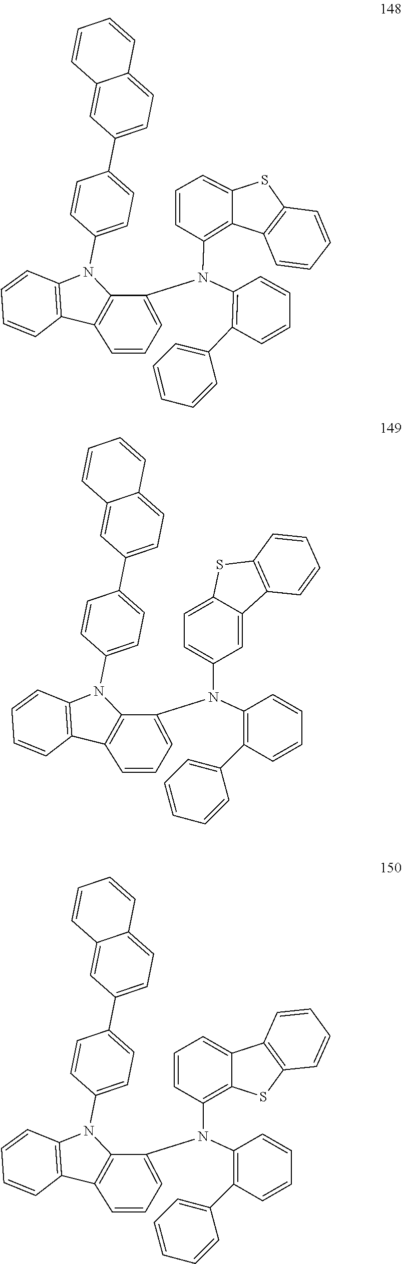 [SMILES: c1ccc(-c2ccccc2N(c2cccc3sc4ccccc4c23)c2cccc3c4ccccc4n(-c4ccc(-c5ccc6ccccc6c5)cc4)c23)cc1 ; c1ccc(-c2ccccc2N(c2ccc3sc4ccccc4c3c2)c2cccc3c4ccccc4n(-c4ccc(-c5ccc6ccccc6c5)cc4)c23)cc1 ; c1ccc(-c2ccccc2N(c2cccc3c2sc2ccccc23)c2cccc3c4ccccc4n(-c4ccc(-c5ccc6ccccc6c5)cc4)c23)cc1] 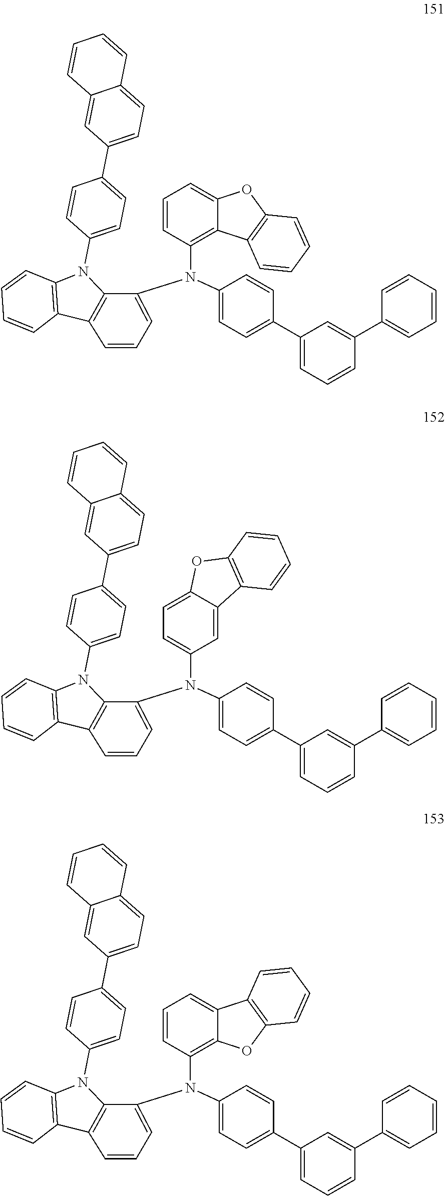 [SMILES: c1ccc(-c2cccc(-c3ccc(N(c4cccc5oc6ccccc6c45)c4cccc5c6ccccc6n(-c6ccc(-c7ccc8ccccc8c7)cc6)c45)cc3)c2)cc1 ; c1ccc(-c2cccc(-c3ccc(N(c4ccc5oc6ccccc6c5c4)c4cccc5c6ccccc6n(-c6ccc(-c7ccc8ccccc8c7)cc6)c45)cc3)c2)cc1 ; c1ccc(-c2cccc(-c3ccc(N(c4cccc5c4oc4ccccc45)c4cccc5c6ccccc6n(-c6ccc(-c7ccc8ccccc8c7)cc6)c45)cc3)c2)cc1] 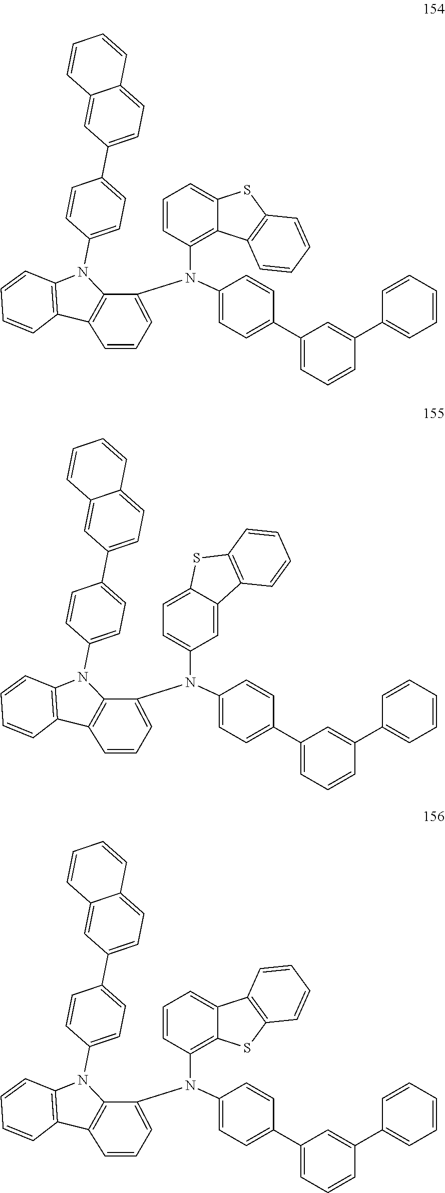 [SMILES: c1ccc(-c2cccc(-c3ccc(N(c4cccc5sc6ccccc6c45)c4cccc5c6ccccc6n(-c6ccc(-c7ccc8ccccc8c7)cc6)c45)cc3)c2)cc1 ; c1ccc(-c2cccc(-c3ccc(N(c4ccc5sc6ccccc6c5c4)c4cccc5c6ccccc6n(-c6ccc(-c7ccc8ccccc8c7)cc6)c45)cc3)c2)cc1 ; c1ccc(-c2cccc(-c3ccc(N(c4cccc5c4sc4ccccc45)c4cccc5c6ccccc6n(-c6ccc(-c7ccc8ccccc8c7)cc6)c45)cc3)c2)cc1] 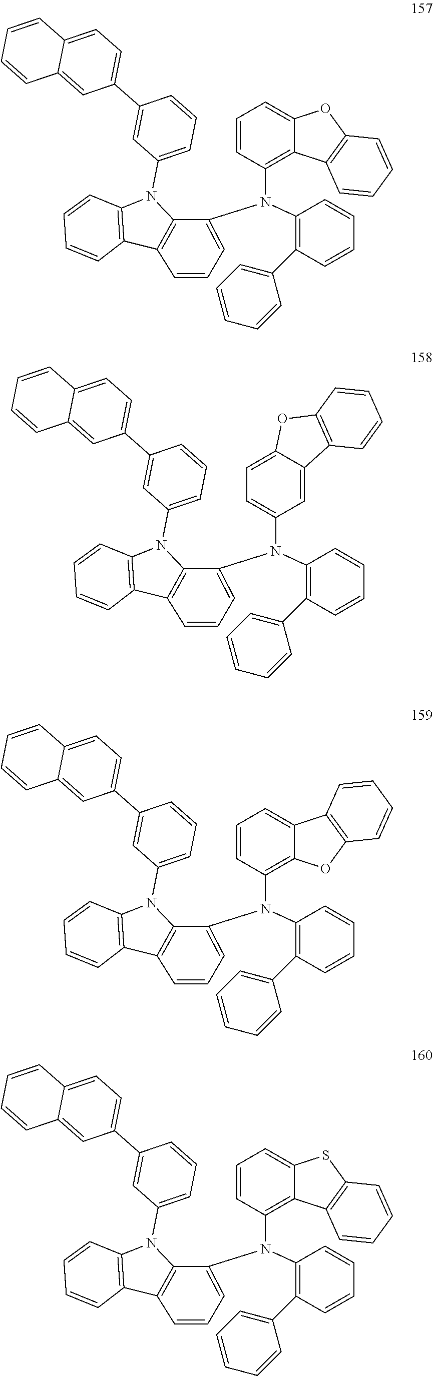 [SMILES: c1ccc(-c2ccccc2N(c2cccc3oc4ccccc4c23)c2cccc3c4ccccc4n(-c4cccc(-c5ccc6ccccc6c5)c4)c23)cc1 ; c1ccc(-c2ccccc2N(c2ccc3oc4ccccc4c3c2)c2cccc3c4ccccc4n(-c4cccc(-c5ccc6ccccc6c5)c4)c23)cc1 ; c1ccc(-c2ccccc2N(c2cccc3c2oc2ccccc23)c2cccc3c4ccccc4n(-c4cccc(-c5ccc6ccccc6c5)c4)c23)cc1 ; c1ccc(-c2ccccc2N(c2cccc3sc4ccccc4c23)c2cccc3c4ccccc4n(-c4cccc(-c5ccc6ccccc6c5)c4)c23)cc1] 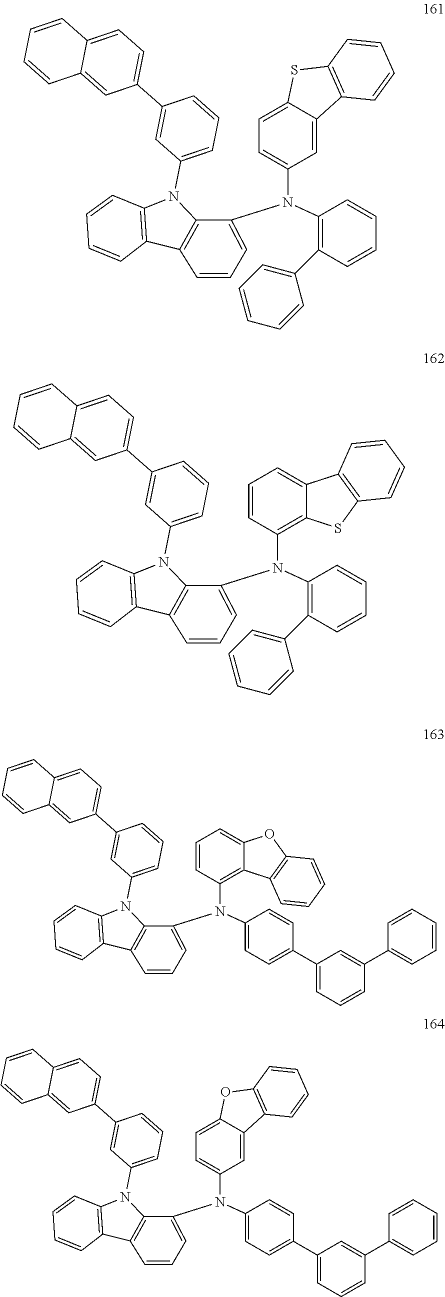 [SMILES: c1ccc(-c2ccccc2N(c2ccc3sc4ccccc4c3c2)c2cccc3c4ccccc4n(-c4cccc(-c5ccc6ccccc6c5)c4)c23)cc1 ; c1ccc(-c2ccccc2N(c2cccc3c2sc2ccccc23)c2cccc3c4ccccc4n(-c4cccc(-c5ccc6ccccc6c5)c4)c23)cc1 ; c1ccc(-c2cccc(-c3ccc(N(c4cccc5oc6ccccc6c45)c4cccc5c6ccccc6n(-c6cccc(-c7ccc8ccccc8c7)c6)c45)cc3)c2)cc1 ; c1ccc(-c2cccc(-c3ccc(N(c4ccc5oc6ccccc6c5c4)c4cccc5c6ccccc6n(-c6cccc(-c7ccc8ccccc8c7)c6)c45)cc3)c2)cc1] 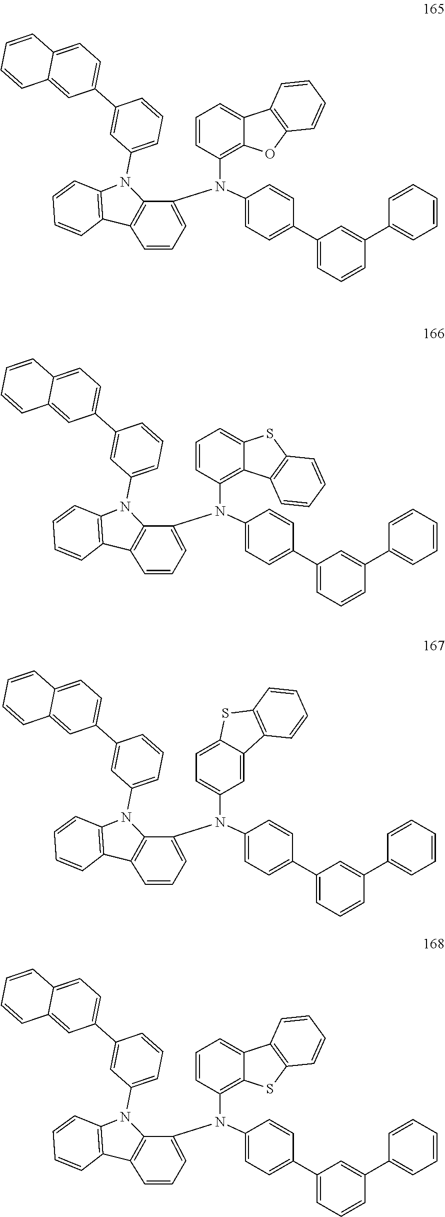 [SMILES: c1ccc(-c2cccc(-c3ccc(N(c4cccc5c4oc4ccccc45)c4cccc5c6ccccc6n(-c6cccc(-c7ccc8ccccc8c7)c6)c45)cc3)c2)cc1 ; c1ccc(-c2cccc(-c3ccc(N(c4cccc5sc6ccccc6c45)c4cccc5c6ccccc6n(-c6cccc(-c7ccc8ccccc8c7)c6)c45)cc3)c2)cc1 ; c1ccc(-c2cccc(-c3ccc(N(c4ccc5sc6ccccc6c5c4)c4cccc5c6ccccc6n(-c6cccc(-c7ccc8ccccc8c7)c6)c45)cc3)c2)cc1 ; c1ccc(-c2cccc(-c3ccc(N(c4cccc5c4sc4ccccc45)c4cccc5c6ccccc6n(-c6cccc(-c7ccc8ccccc8c7)c6)c45)cc3)c2)cc1] 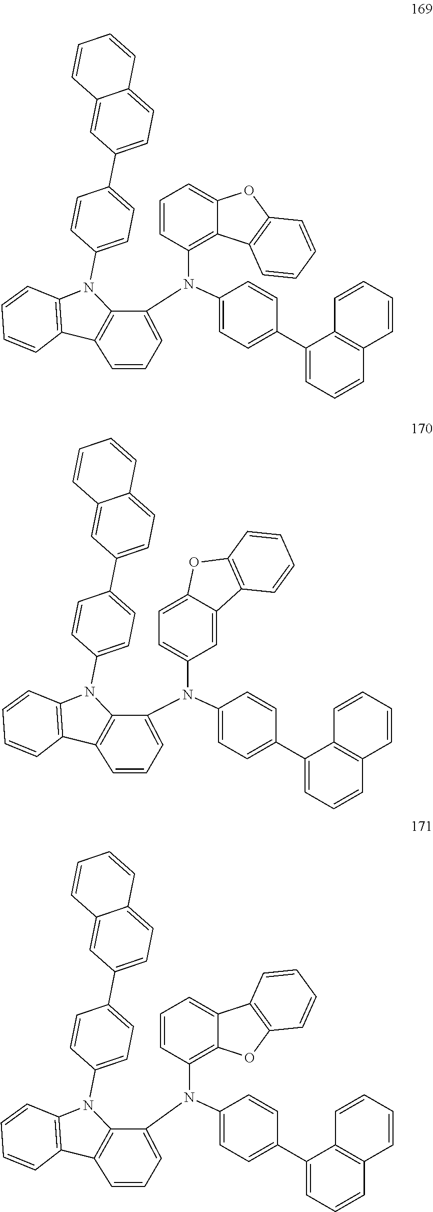 [SMILES: c1ccc2cc(-c3ccc(-n4c5ccccc5c5cccc(N(c6ccc(-c7cccc8ccccc78)cc6)c6cccc7oc8ccccc8c67)c54)cc3)ccc2c1 ; c1ccc2cc(-c3ccc(-n4c5ccccc5c5cccc(N(c6ccc(-c7cccc8ccccc78)cc6)c6ccc7oc8ccccc8c7c6)c54)cc3)ccc2c1 ; c1ccc2cc(-c3ccc(-n4c5ccccc5c5cccc(N(c6ccc(-c7cccc8ccccc78)cc6)c6cccc7c6oc6ccccc67)c54)cc3)ccc2c1] 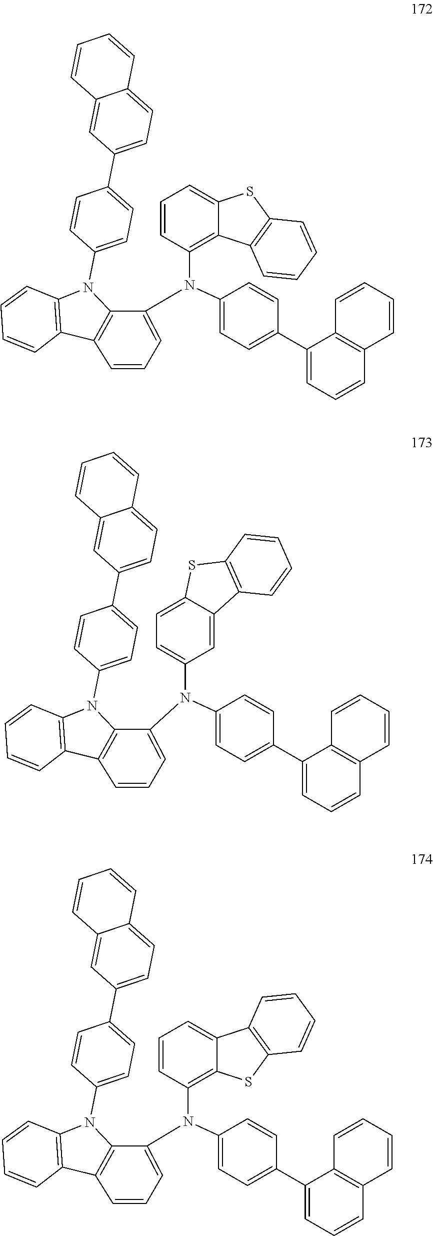 [SMILES: c1ccc2cc(-c3ccc(-n4c5ccccc5c5cccc(N(c6ccc(-c7cccc8ccccc78)cc6)c6cccc7sc8ccccc8c67)c54)cc3)ccc2c1 ; c1ccc2cc(-c3ccc(-n4c5ccccc5c5cccc(N(c6ccc(-c7cccc8ccccc78)cc6)c6ccc7sc8ccccc8c7c6)c54)cc3)ccc2c1 ; c1ccc2cc(-c3ccc(-n4c5ccccc5c5cccc(N(c6ccc(-c7cccc8ccccc78)cc6)c6cccc7c6sc6ccccc67)c54)cc3)ccc2c1] 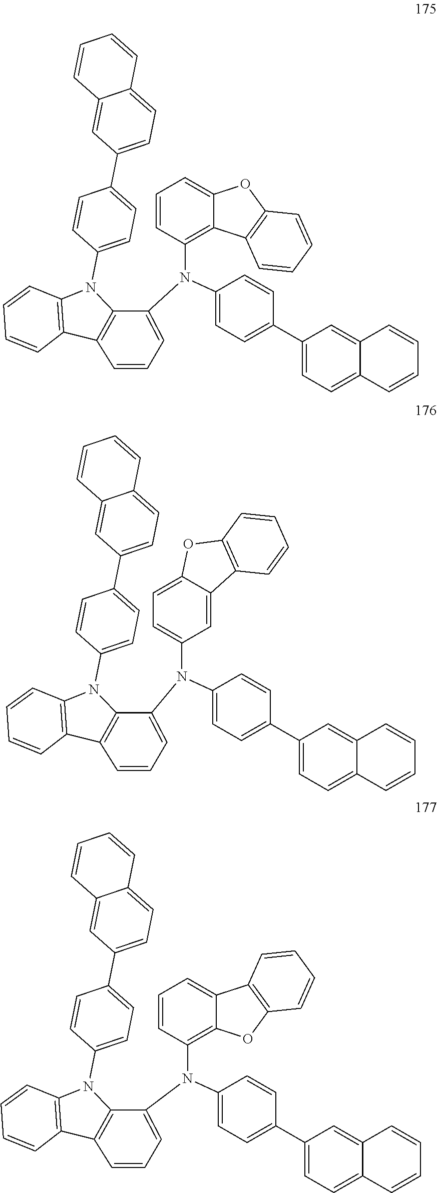 [SMILES: c1ccc2cc(-c3ccc(N(c4cccc5oc6ccccc6c45)c4cccc5c6ccccc6n(-c6ccc(-c7ccc8ccccc8c7)cc6)c45)cc3)ccc2c1 ; c1ccc2cc(-c3ccc(N(c4ccc5oc6ccccc6c5c4)c4cccc5c6ccccc6n(-c6ccc(-c7ccc8ccccc8c7)cc6)c45)cc3)ccc2c1 ; c1ccc2cc(-c3ccc(N(c4cccc5c4oc4ccccc45)c4cccc5c6ccccc6n(-c6ccc(-c7ccc8ccccc8c7)cc6)c45)cc3)ccc2c1] 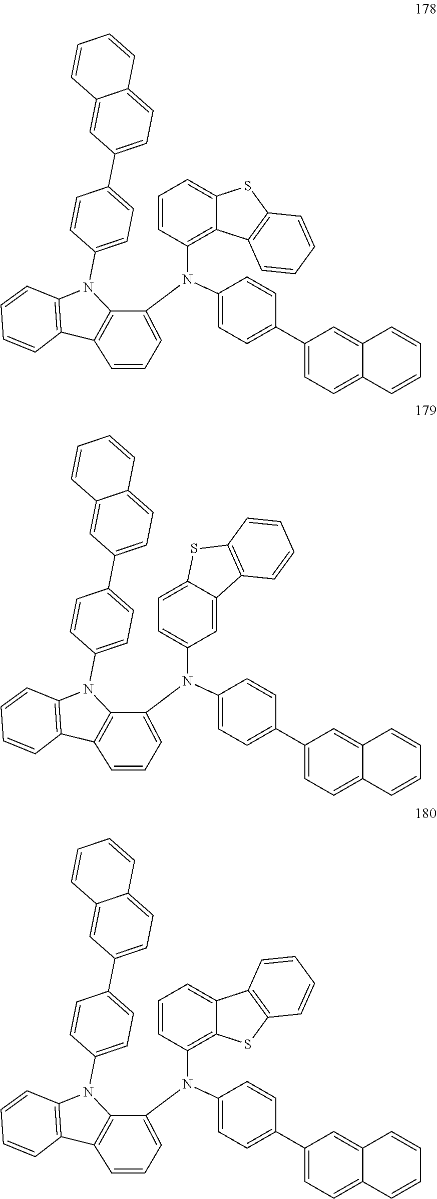 [SMILES: c1ccc2cc(-c3ccc(N(c4cccc5sc6ccccc6c45)c4cccc5c6ccccc6n(-c6ccc(-c7ccc8ccccc8c7)cc6)c45)cc3)ccc2c1 ; c1ccc2cc(-c3ccc(N(c4ccc5sc6ccccc6c5c4)c4cccc5c6ccccc6n(-c6ccc(-c7ccc8ccccc8c7)cc6)c45)cc3)ccc2c1 ; c1ccc2cc(-c3ccc(N(c4cccc5c4sc4ccccc45)c4cccc5c6ccccc6n(-c6ccc(-c7ccc8ccccc8c7)cc6)c45)cc3)ccc2c1] 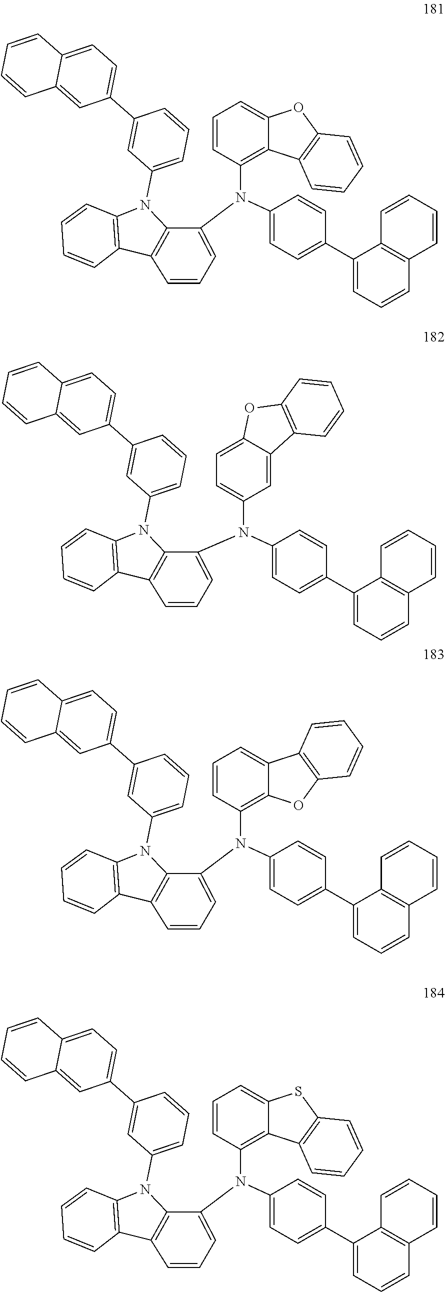 [SMILES: c1cc(-c2ccc3ccccc3c2)cc(-n2c3ccccc3c3cccc(N(c4ccc(-c5cccc6ccccc56)cc4)c4cccc5oc6ccccc6c45)c32)c1 ; c1cc(-c2ccc3ccccc3c2)cc(-n2c3ccccc3c3cccc(N(c4ccc(-c5cccc6ccccc56)cc4)c4ccc5oc6ccccc6c5c4)c32)c1 ; c1cc(-c2ccc3ccccc3c2)cc(-n2c3ccccc3c3cccc(N(c4ccc(-c5cccc6ccccc56)cc4)c4cccc5c4oc4ccccc45)c32)c1 ; c1cc(-c2ccc3ccccc3c2)cc(-n2c3ccccc3c3cccc(N(c4ccc(-c5cccc6ccccc56)cc4)c4cccc5sc6ccccc6c45)c32)c1] 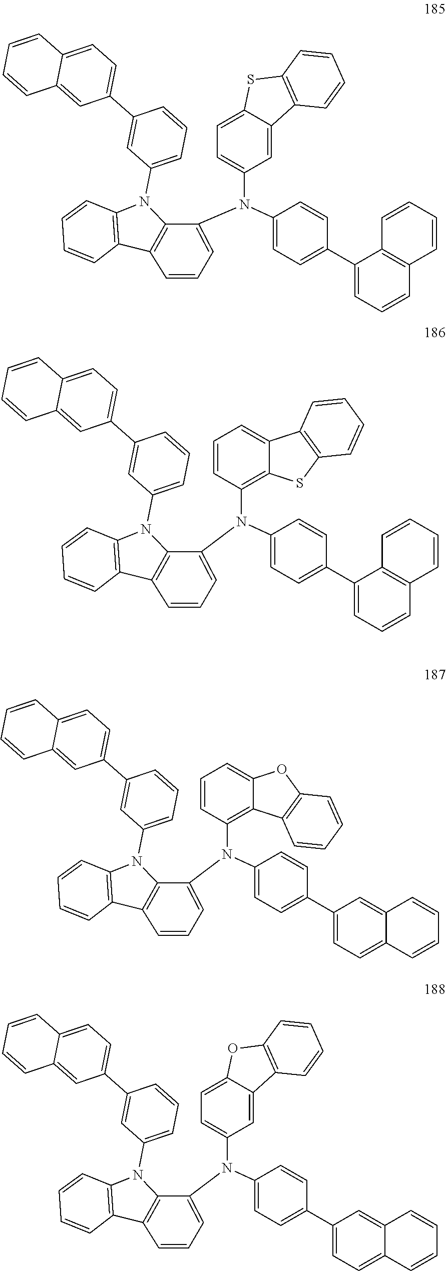 [SMILES: c1cc(-c2ccc3ccccc3c2)cc(-n2c3ccccc3c3cccc(N(c4ccc(-c5cccc6ccccc56)cc4)c4ccc5sc6ccccc6c5c4)c32)c1 ; c1cc(-c2ccc3ccccc3c2)cc(-n2c3ccccc3c3cccc(N(c4ccc(-c5cccc6ccccc56)cc4)c4cccc5c4sc4ccccc45)c32)c1 ; c1cc(-c2ccc3ccccc3c2)cc(-n2c3ccccc3c3cccc(N(c4ccc(-c5ccc6ccccc6c5)cc4)c4cccc5oc6ccccc6c45)c32)c1 ; c1cc(-c2ccc3ccccc3c2)cc(-n2c3ccccc3c3cccc(N(c4ccc(-c5ccc6ccccc6c5)cc4)c4ccc5oc6ccccc6c5c4)c32)c1] 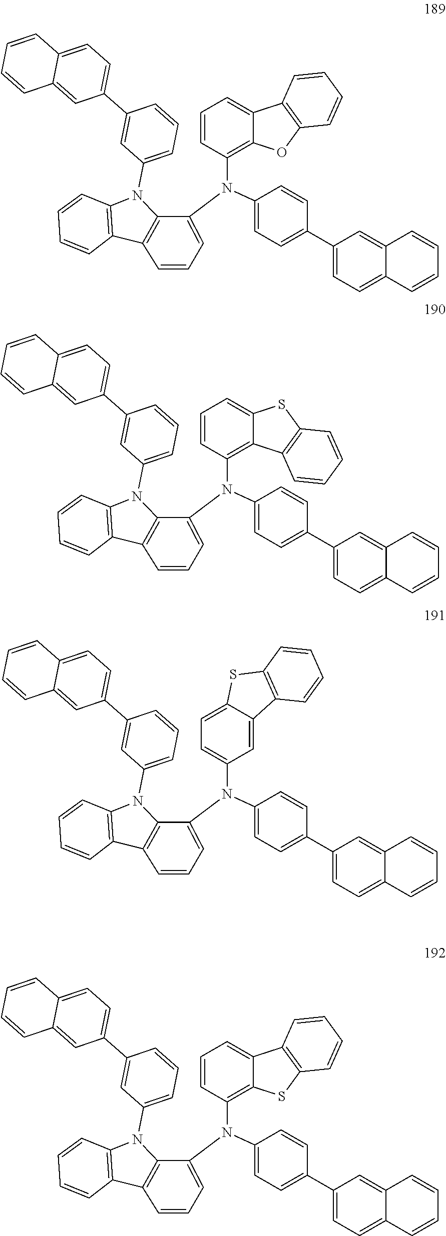 [SMILES: c1cc(-c2ccc3ccccc3c2)cc(-n2c3ccccc3c3cccc(N(c4ccc(-c5ccc6ccccc6c5)cc4)c4cccc5c4oc4ccccc45)c32)c1 ; c1cc(-c2ccc3ccccc3c2)cc(-n2c3ccccc3c3cccc(N(c4ccc(-c5ccc6ccccc6c5)cc4)c4cccc5sc6ccccc6c45)c32)c1 ; c1cc(-c2ccc3ccccc3c2)cc(-n2c3ccccc3c3cccc(N(c4ccc(-c5ccc6ccccc6c5)cc4)c4ccc5sc6ccccc6c5c4)c32)c1 ; c1cc(-c2ccc3ccccc3c2)cc(-n2c3ccccc3c3cccc(N(c4ccc(-c5ccc6ccccc6c5)cc4)c4cccc5c4sc4ccccc45)c32)c1] 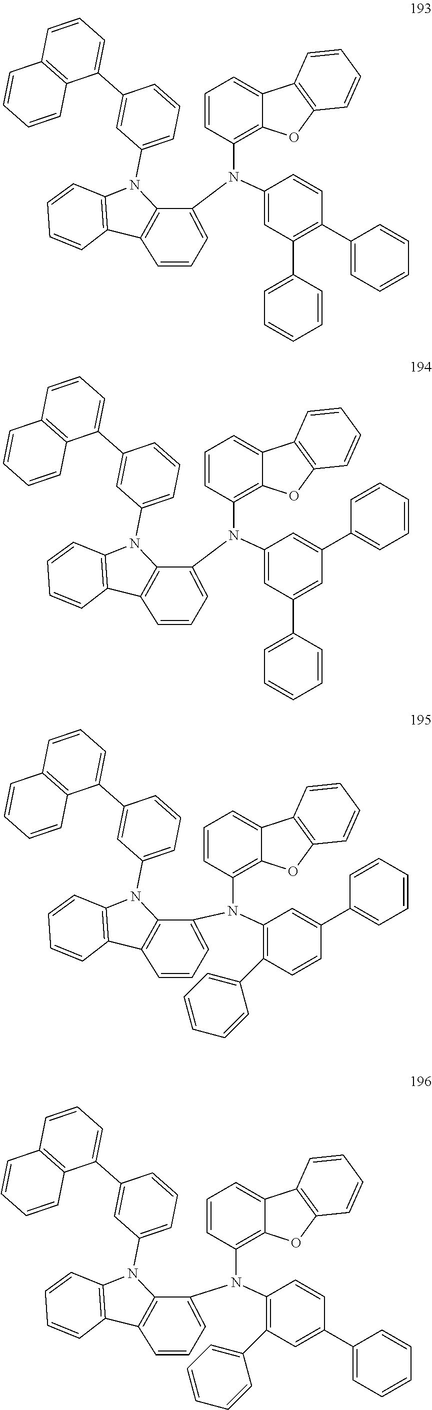 [SMILES: c1ccc(-c2ccc(N(c3cccc4c3oc3ccccc34)c3cccc4c5ccccc5n(-c5cccc(-c6cccc7ccccc67)c5)c34)cc2-c2ccccc2)cc1 ; c1ccc(-c2cc(-c3ccccc3)cc(N(c3cccc4c3oc3ccccc34)c3cccc4c5ccccc5n(-c5cccc(-c6cccc7ccccc67)c5)c34)c2)cc1 ; c1ccc(-c2ccc(-c3ccccc3)c(N(c3cccc4c3oc3ccccc34)c3cccc4c5ccccc5n(-c5cccc(-c6cccc7ccccc67)c5)c34)c2)cc1 ; c1ccc(-c2ccc(N(c3cccc4c3oc3ccccc34)c3cccc4c5ccccc5n(-c5cccc(-c6cccc7ccccc67)c5)c34)c(-c3ccccc3)c2)cc1] 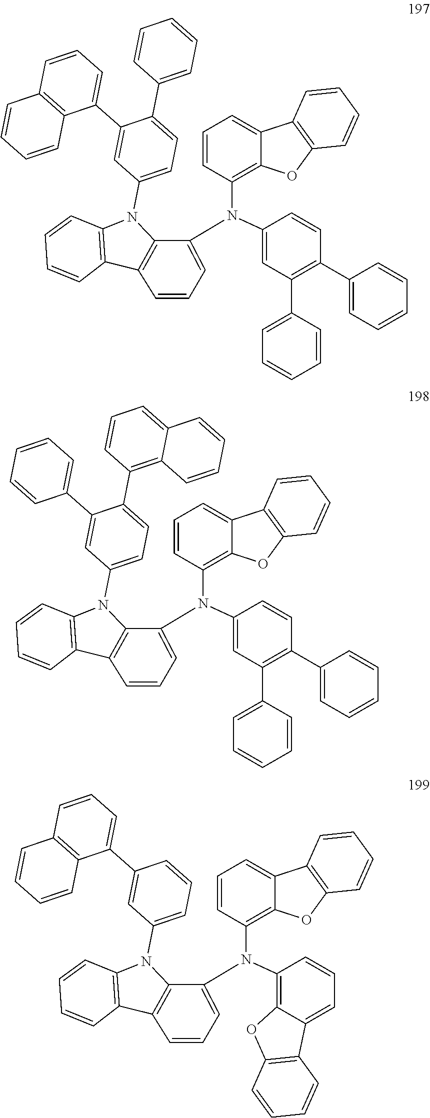 [SMILES: c1ccc(-c2ccc(N(c3cccc4c3oc3ccccc34)c3cccc4c5ccccc5n(-c5ccc(-c6ccccc6)c(-c6cccc7ccccc67)c5)c34)cc2-c2ccccc2)cc1 ; c1ccc(-c2ccc(N(c3cccc4c3oc3ccccc34)c3cccc4c5ccccc5n(-c5ccc(-c6cccc7ccccc67)c(-c6ccccc6)c5)c34)cc2-c2ccccc2)cc1 ; c1cc(-c2cccc3ccccc23)cc(-n2c3ccccc3c3cccc(N(c4cccc5c4oc4ccccc45)c4cccc5c4oc4ccccc45)c32)c1] 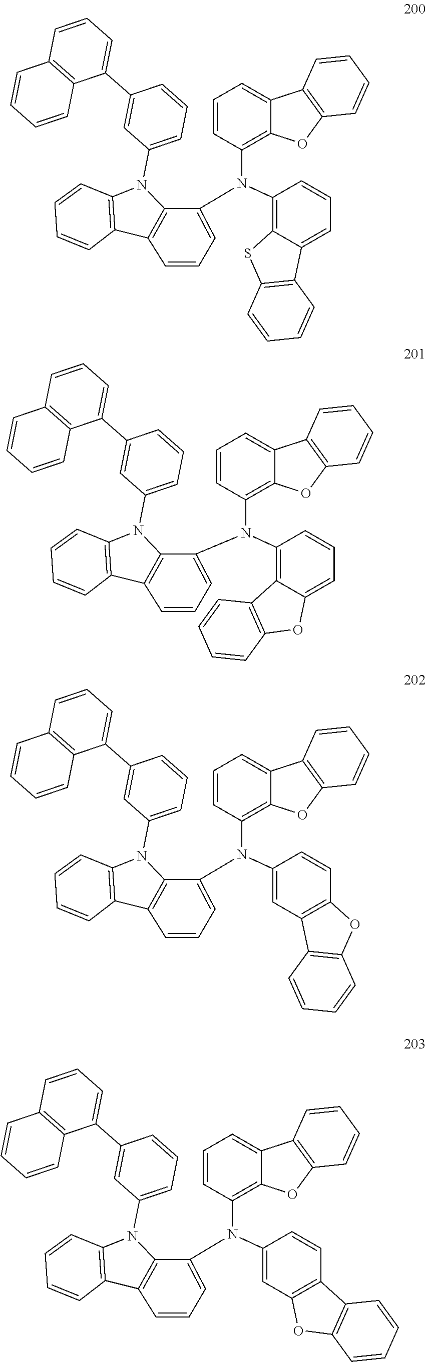 [SMILES: c1cc(-c2cccc3ccccc23)cc(-n2c3ccccc3c3cccc(N(c4cccc5c4oc4ccccc45)c4cccc5c4sc4ccccc45)c32)c1 ; c1cc(-c2cccc3ccccc23)cc(-n2c3ccccc3c3cccc(N(c4cccc5c4oc4ccccc45)c4cccc5oc6ccccc6c45)c32)c1 ; c1cc(-c2cccc3ccccc23)cc(-n2c3ccccc3c3cccc(N(c4ccc5oc6ccccc6c5c4)c4cccc5c4oc4ccccc45)c32)c1 ; c1cc(-c2cccc3ccccc23)cc(-n2c3ccccc3c3cccc(N(c4ccc5c(c4)oc4ccccc45)c4cccc5c4oc4ccccc45)c32)c1] 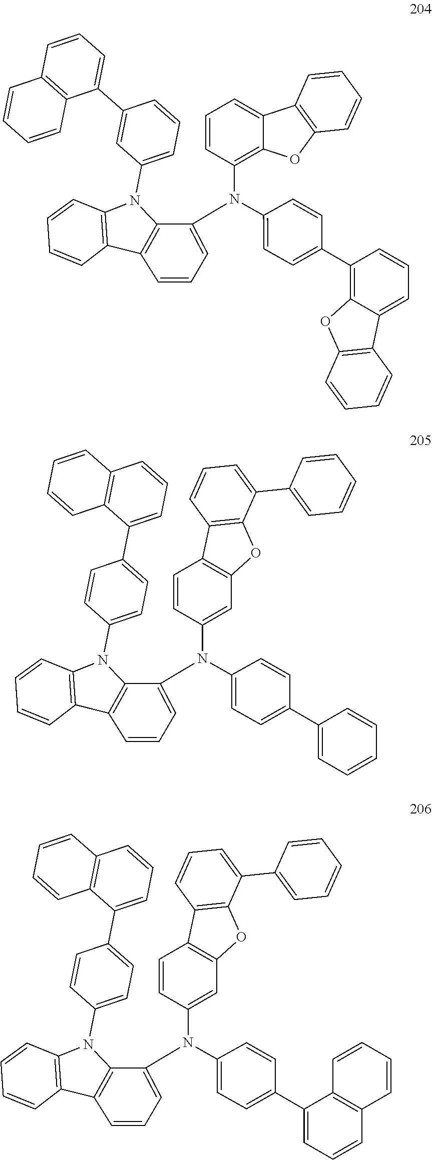 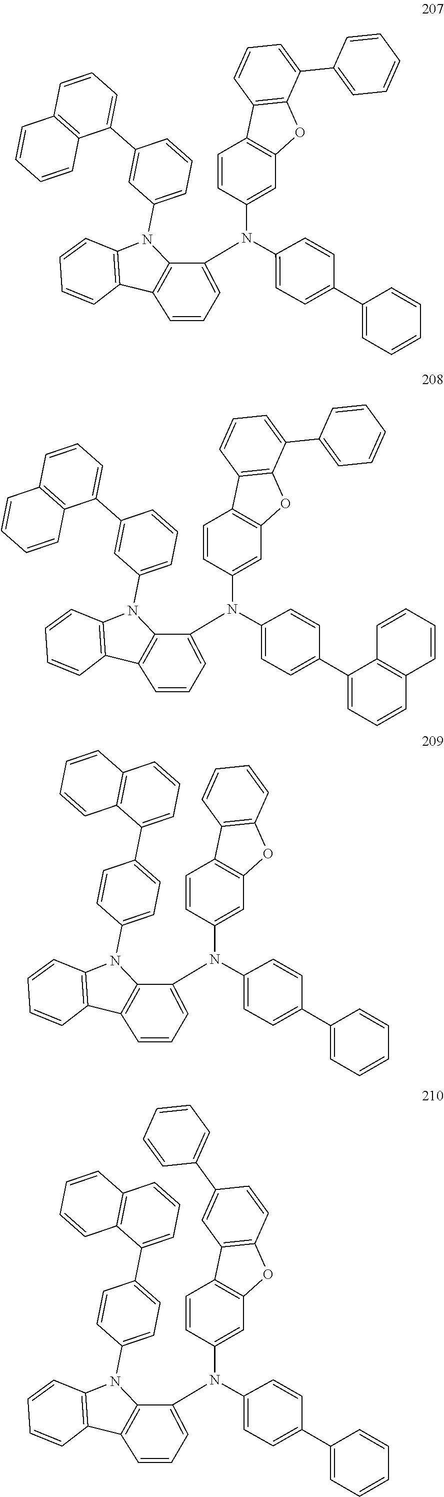 [SMILES: c1ccc(-c2ccc(N(c3ccc4c(c3)oc3c(-c5ccccc5)cccc34)c3cccc4c5ccccc5n(-c5cccc(-c6cccc7ccccc67)c5)c34)cc2)cc1 ; c1ccc(-c2cccc3c2oc2cc(N(c4ccc(-c5cccc6ccccc56)cc4)c4cccc5c6ccccc6n(-c6cccc(-c7cccc8ccccc78)c6)c45)ccc23)cc1 ; c1ccc(-c2ccc(N(c3ccc4c(c3)oc3ccccc34)c3cccc4c5ccccc5n(-c5ccc(-c6cccc7ccccc67)cc5)c34)cc2)cc1 ; c1ccc(-c2ccc(N(c3ccc4c(c3)oc3ccc(-c5ccccc5)cc34)c3cccc4c5ccccc5n(-c5ccc(-c6cccc7ccccc67)cc5)c34)cc2)cc1] 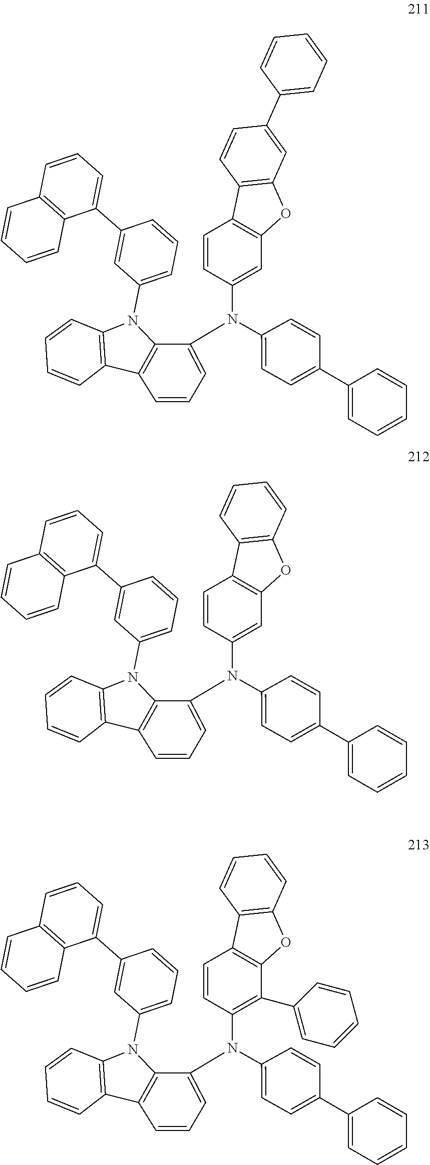 [SMILES: c1ccc(-c2ccc(N(c3ccc4c(c3)oc3cc(-c5ccccc5)ccc34)c3cccc4c5ccccc5n(-c5cccc(-c6cccc7ccccc67)c5)c34)cc2)cc1 ; c1ccc(-c2ccc(N(c3ccc4c(c3)oc3ccccc34)c3cccc4c5ccccc5n(-c5cccc(-c6cccc7ccccc67)c5)c34)cc2)cc1 ; c1ccc(-c2ccc(N(c3ccc4c(oc5ccccc54)c3-c3ccccc3)c3cccc4c5ccccc5n(-c5cccc(-c6cccc7ccccc67)c5)c34)cc2)cc1] 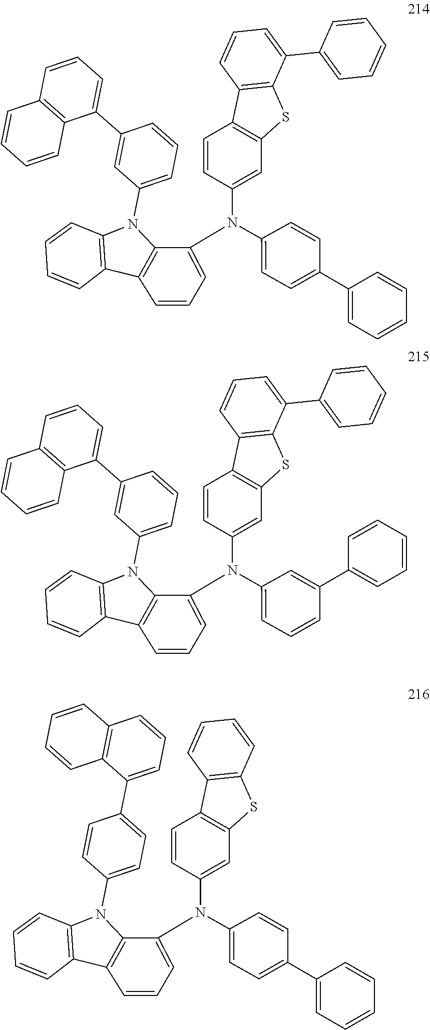 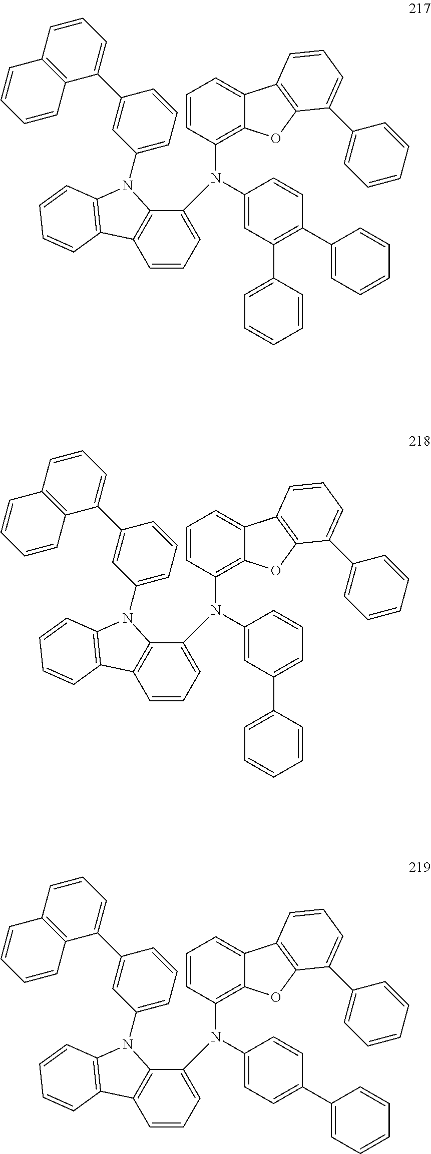 [SMILES: c1ccc(-c2ccc(N(c3cccc4c3oc3c(-c5ccccc5)cccc34)c3cccc4c5ccccc5n(-c5cccc(-c6cccc7ccccc67)c5)c34)cc2-c2ccccc2)cc1 ; c1ccc(-c2cccc(N(c3cccc4c3oc3c(-c5ccccc5)cccc34)c3cccc4c5ccccc5n(-c5cccc(-c6cccc7ccccc67)c5)c34)c2)cc1 ; c1ccc(-c2ccc(N(c3cccc4c3oc3c(-c5ccccc5)cccc34)c3cccc4c5ccccc5n(-c5cccc(-c6cccc7ccccc67)c5)c34)cc2)cc1] 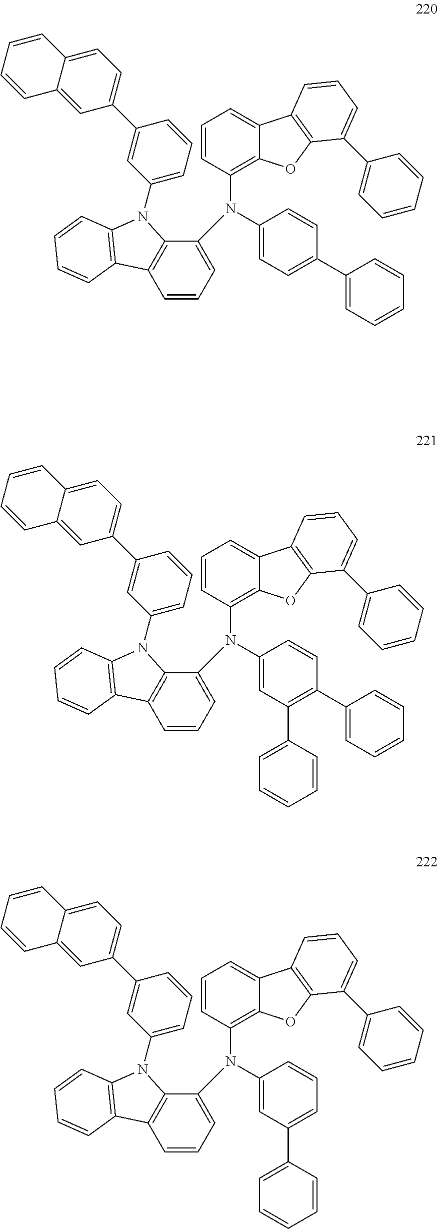 [SMILES: c1ccc(-c2ccc(N(c3cccc4c3oc3c(-c5ccccc5)cccc34)c3cccc4c5ccccc5n(-c5cccc(-c6ccc7ccccc7c6)c5)c34)cc2)cc1 ; c1ccc(-c2ccc(N(c3cccc4c3oc3c(-c5ccccc5)cccc34)c3cccc4c5ccccc5n(-c5cccc(-c6ccc7ccccc7c6)c5)c34)cc2-c2ccccc2)cc1 ; c1ccc(-c2cccc(N(c3cccc4c3oc3c(-c5ccccc5)cccc34)c3cccc4c5ccccc5n(-c5cccc(-c6ccc7ccccc7c6)c5)c34)c2)cc1] 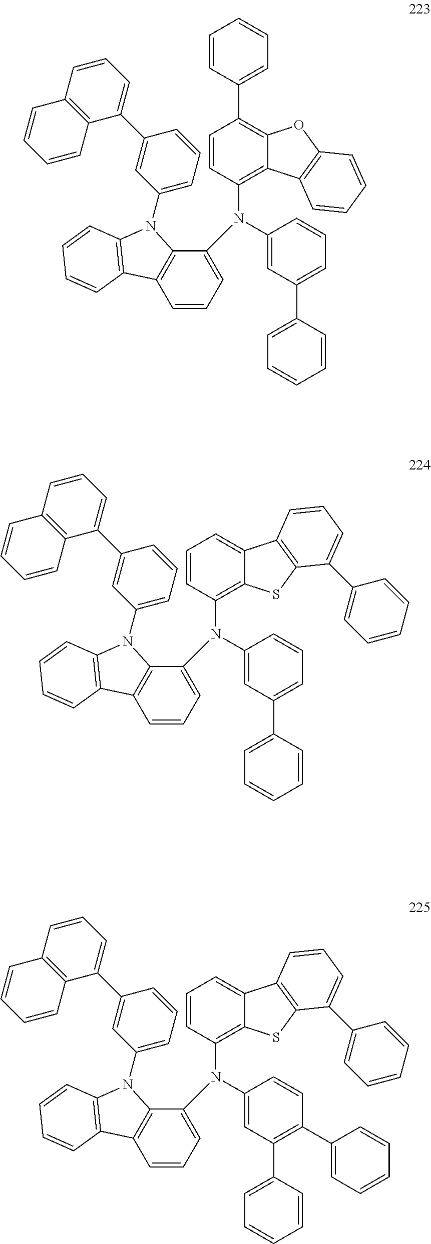 [SMILES: c1ccc(-c2cccc(N(c3ccc(-c4ccccc4)c4oc5ccccc5c34)c3cccc4c5ccccc5n(-c5cccc(-c6cccc7ccccc67)c5)c34)c2)cc1 ; c1ccc(-c2cccc(N(c3cccc4c3sc3c(-c5ccccc5)cccc34)c3cccc4c5ccccc5n(-c5cccc(-c6cccc7ccccc67)c5)c34)c2)cc1 ; c1ccc(-c2ccc(N(c3cccc4c3sc3c(-c5ccccc5)cccc34)c3cccc4c5ccccc5n(-c5cccc(-c6cccc7ccccc67)c5)c34)cc2-c2ccccc2)cc1] 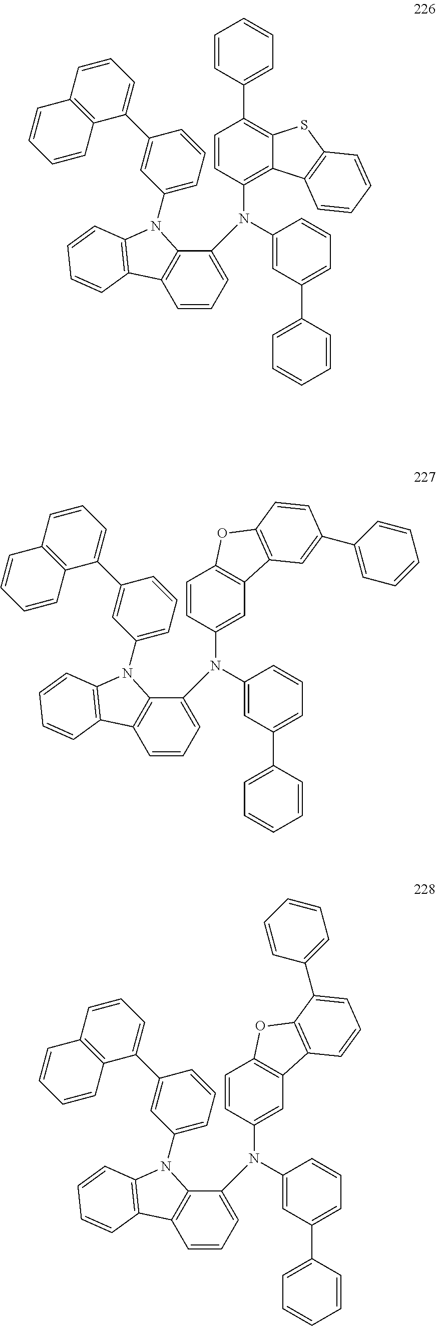 [SMILES: c1ccc(-c2cccc(N(c3ccc(-c4ccccc4)c4sc5ccccc5c34)c3cccc4c5ccccc5n(-c5cccc(-c6cccc7ccccc67)c5)c34)c2)cc1 ; c1ccc(-c2cccc(N(c3ccc4oc5ccc(-c6ccccc6)cc5c4c3)c3cccc4c5ccccc5n(-c5cccc(-c6cccc7ccccc67)c5)c34)c2)cc1 ; c1ccc(-c2cccc(N(c3ccc4oc5c(-c6ccccc6)cccc5c4c3)c3cccc4c5ccccc5n(-c5cccc(-c6cccc7ccccc67)c5)c34)c2)cc1] 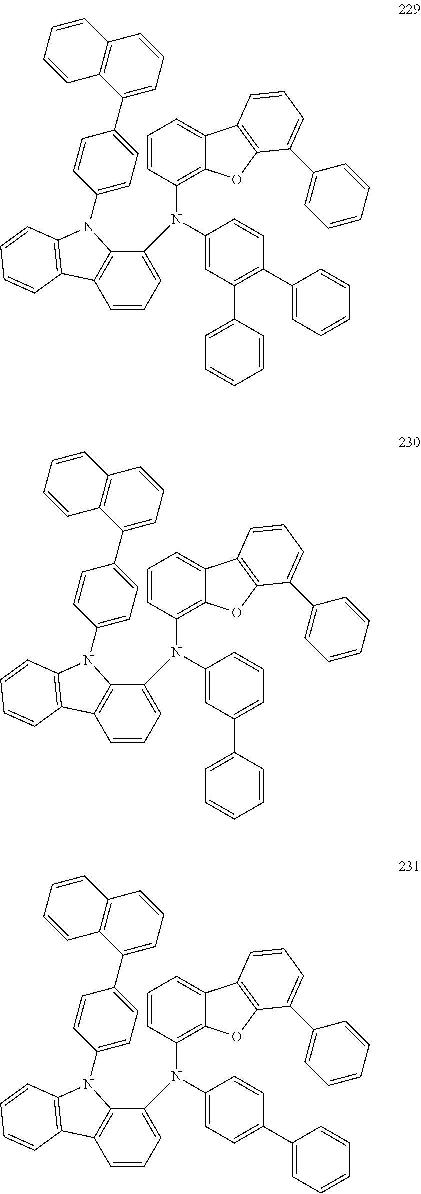 [SMILES: c1ccc(-c2ccc(N(c3cccc4c3oc3c(-c5ccccc5)cccc34)c3cccc4c5ccccc5n(-c5ccc(-c6cccc7ccccc67)cc5)c34)cc2-c2ccccc2)cc1 ; c1ccc(-c2cccc(N(c3cccc4c3oc3c(-c5ccccc5)cccc34)c3cccc4c5ccccc5n(-c5ccc(-c6cccc7ccccc67)cc5)c34)c2)cc1 ; c1ccc(-c2ccc(N(c3cccc4c3oc3c(-c5ccccc5)cccc34)c3cccc4c5ccccc5n(-c5ccc(-c6cccc7ccccc67)cc5)c34)cc2)cc1] 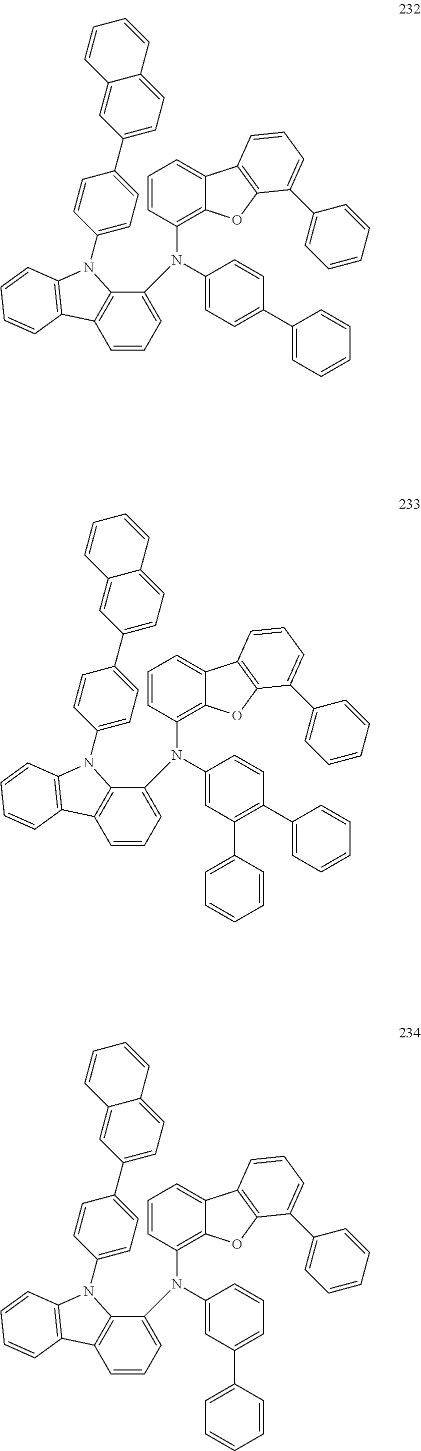 [SMILES: c1ccc(-c2ccc(N(c3cccc4c3oc3c(-c5ccccc5)cccc34)c3cccc4c5ccccc5n(-c5ccc(-c6ccc7ccccc7c6)cc5)c34)cc2)cc1 ; c1ccc(-c2ccc(N(c3cccc4c3oc3c(-c5ccccc5)cccc34)c3cccc4c5ccccc5n(-c5ccc(-c6ccc7ccccc7c6)cc5)c34)cc2-c2ccccc2)cc1 ; c1ccc(-c2cccc(N(c3cccc4c3oc3c(-c5ccccc5)cccc34)c3cccc4c5ccccc5n(-c5ccc(-c6ccc7ccccc7c6)cc5)c34)c2)cc1] 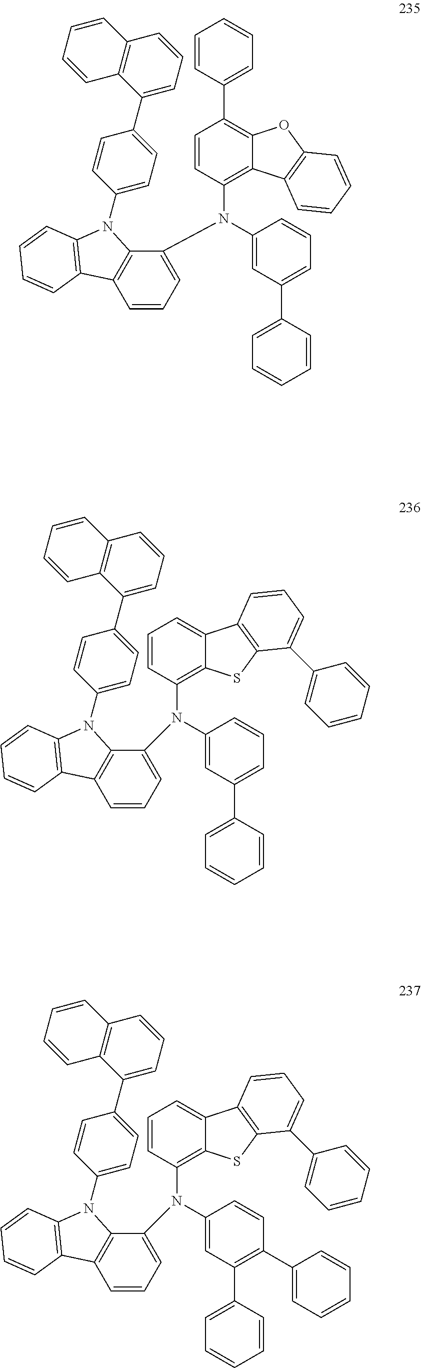 [SMILES: c1ccc(-c2cccc(N(c3ccc(-c4ccccc4)c4oc5ccccc5c34)c3cccc4c5ccccc5n(-c5ccc(-c6cccc7ccccc67)cc5)c34)c2)cc1 ; c1ccc(-c2cccc(N(c3cccc4c3sc3c(-c5ccccc5)cccc34)c3cccc4c5ccccc5n(-c5ccc(-c6cccc7ccccc67)cc5)c34)c2)cc1 ; c1ccc(-c2ccc(N(c3cccc4c3sc3c(-c5ccccc5)cccc34)c3cccc4c5ccccc5n(-c5ccc(-c6cccc7ccccc67)cc5)c34)cc2-c2ccccc2)cc1] 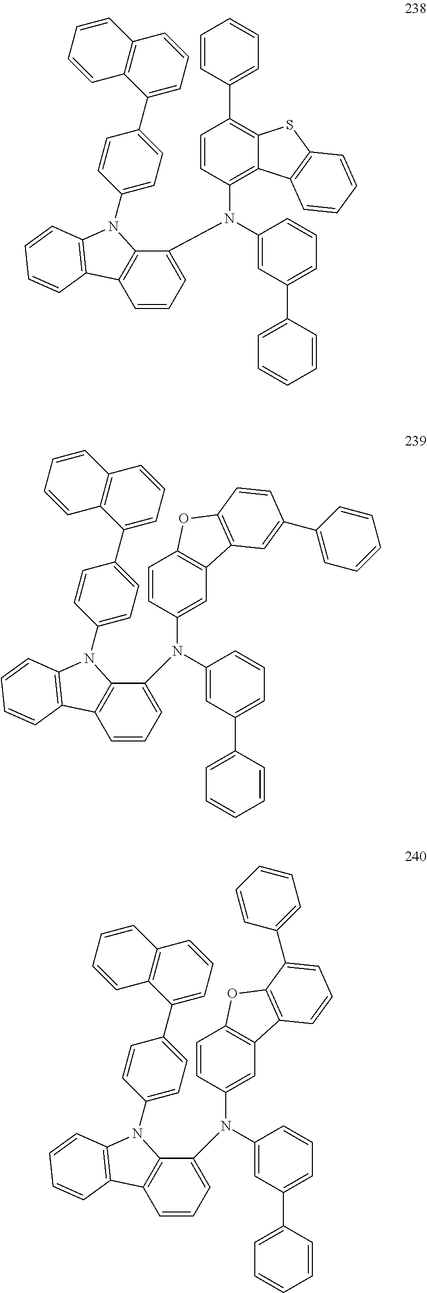 [SMILES: c1ccc(-c2cccc(N(c3ccc(-c4ccccc4)c4sc5ccccc5c34)c3cccc4c5ccccc5n(-c5ccc(-c6cccc7ccccc67)cc5)c34)c2)cc1 ; c1ccc(-c2cccc(N(c3ccc4oc5ccc(-c6ccccc6)cc5c4c3)c3cccc4c5ccccc5n(-c5ccc(-c6cccc7ccccc67)cc5)c34)c2)cc1 ; c1ccc(-c2cccc(N(c3ccc4oc5c(-c6ccccc6)cccc5c4c3)c3cccc4c5ccccc5n(-c5ccc(-c6cccc7ccccc67)cc5)c34)c2)cc1] 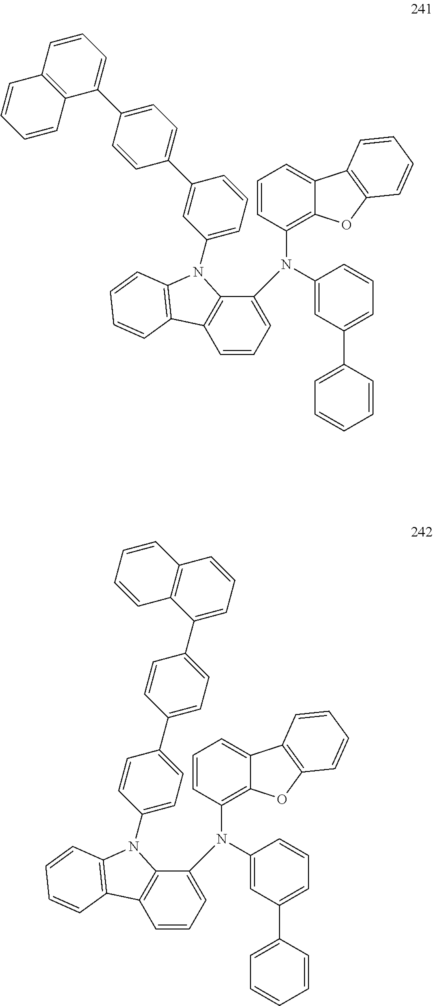 [SMILES: c1ccc(-c2cccc(N(c3cccc4c3oc3ccccc34)c3cccc4c5ccccc5n(-c5cccc(-c6ccc(-c7cccc8ccccc78)cc6)c5)c34)c2)cc1 ; c1ccc(-c2cccc(N(c3cccc4c3oc3ccccc34)c3cccc4c5ccccc5n(-c5ccc(-c6ccc(-c7cccc8ccccc78)cc6)cc5)c34)c2)cc1] 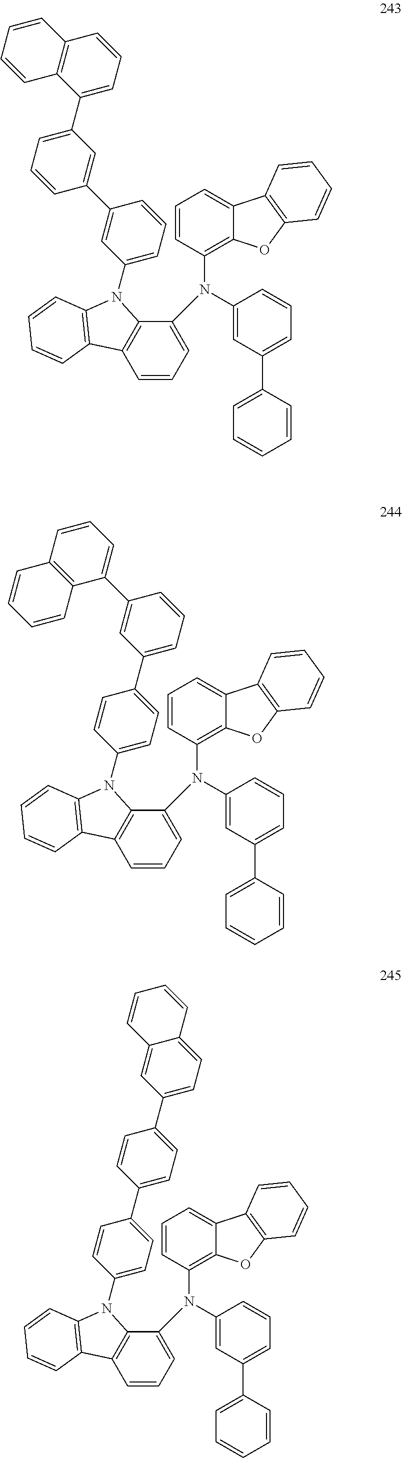 [SMILES: c1ccc(-c2cccc(N(c3cccc4c3oc3ccccc34)c3cccc4c5ccccc5n(-c5cccc(-c6cccc(-c7cccc8ccccc78)c6)c5)c34)c2)cc1 ; c1ccc(-c2cccc(N(c3cccc4c3oc3ccccc34)c3cccc4c5ccccc5n(-c5ccc(-c6cccc(-c7cccc8ccccc78)c6)cc5)c34)c2)cc1 ; c1ccc(-c2cccc(N(c3cccc4c3oc3ccccc34)c3cccc4c5ccccc5n(-c5ccc(-c6ccc(-c7ccc8ccccc8c7)cc6)cc5)c34)c2)cc1] 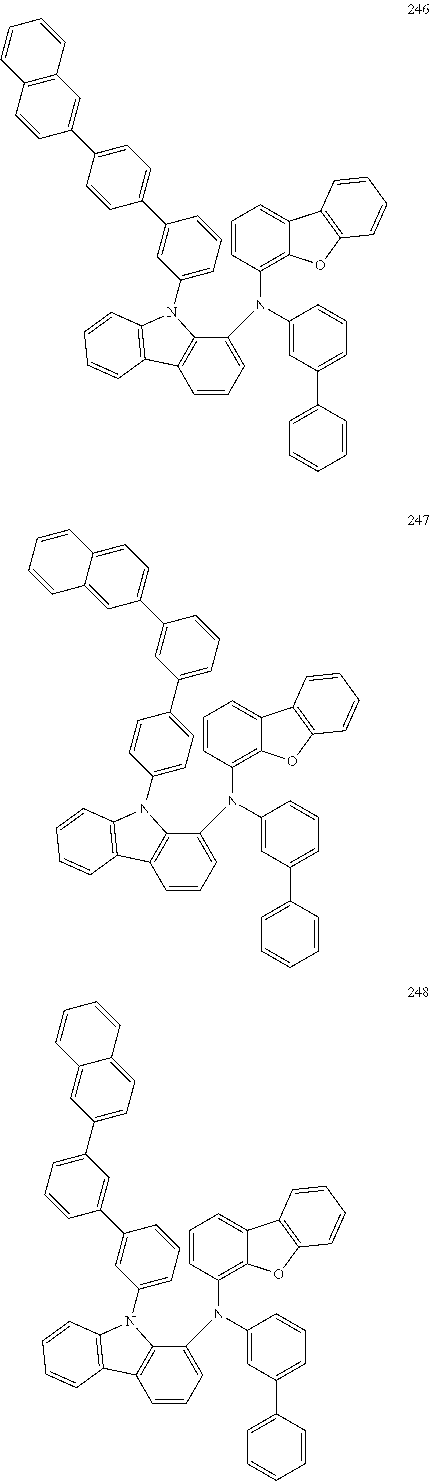 [SMILES: c1ccc(-c2cccc(N(c3cccc4c3oc3ccccc34)c3cccc4c5ccccc5n(-c5cccc(-c6ccc(-c7ccc8ccccc8c7)cc6)c5)c34)c2)cc1 ; c1ccc(-c2cccc(N(c3cccc4c3oc3ccccc34)c3cccc4c5ccccc5n(-c5ccc(-c6cccc(-c7ccc8ccccc8c7)c6)cc5)c34)c2)cc1 ; c1ccc(-c2cccc(N(c3cccc4c3oc3ccccc34)c3cccc4c5ccccc5n(-c5cccc(-c6cccc(-c7ccc8ccccc8c7)c6)c5)c34)c2)cc1] 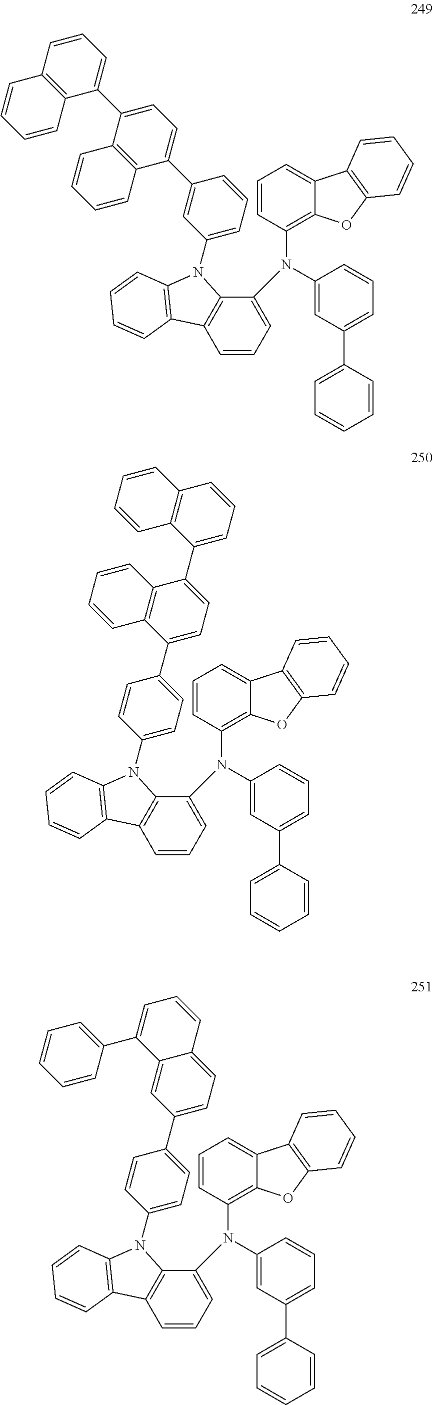 [SMILES: c1ccc(-c2cccc(N(c3cccc4c3oc3ccccc34)c3cccc4c5ccccc5n(-c5cccc(-c6ccc(-c7cccc8ccccc78)c7ccccc67)c5)c34)c2)cc1 ; c1ccc(-c2cccc(N(c3cccc4c3oc3ccccc34)c3cccc4c5ccccc5n(-c5ccc(-c6ccc(-c7cccc8ccccc78)c7ccccc67)cc5)c34)c2)cc1 ; c1ccc(-c2cccc(N(c3cccc4c3oc3ccccc34)c3cccc4c5ccccc5n(-c5ccc(-c6ccc7cccc(-c8ccccc8)c7c6)cc5)c34)c2)cc1] 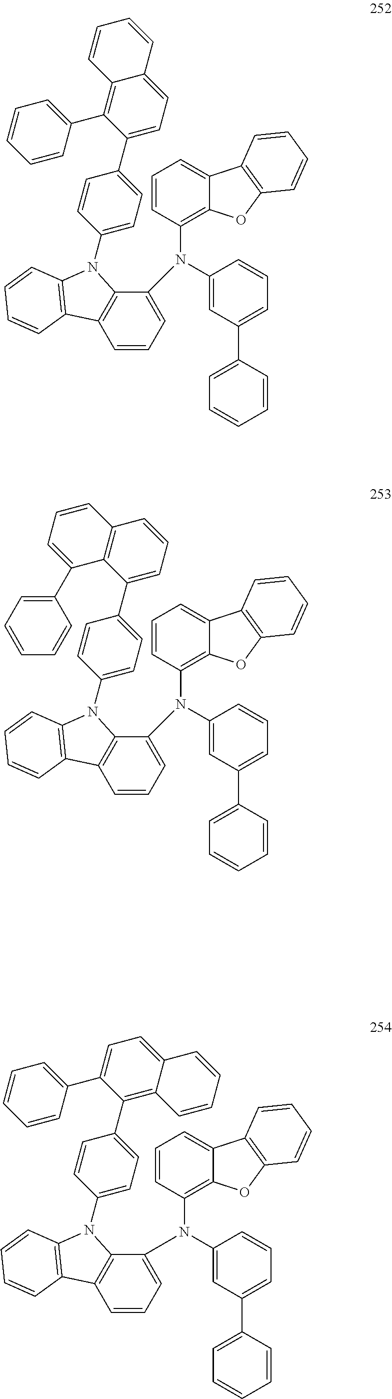 [SMILES: c1ccc(-c2cccc(N(c3cccc4c3oc3ccccc34)c3cccc4c5ccccc5n(-c5ccc(-c6ccc7ccccc7c6-c6ccccc6)cc5)c34)c2)cc1 ; c1ccc(-c2cccc(N(c3cccc4c3oc3ccccc34)c3cccc4c5ccccc5n(-c5ccc(-c6cccc7cccc(-c8ccccc8)c67)cc5)c34)c2)cc1 ; c1ccc(-c2cccc(N(c3cccc4c3oc3ccccc34)c3cccc4c5ccccc5n(-c5ccc(-c6c(-c7ccccc7)ccc7ccccc67)cc5)c34)c2)cc1] 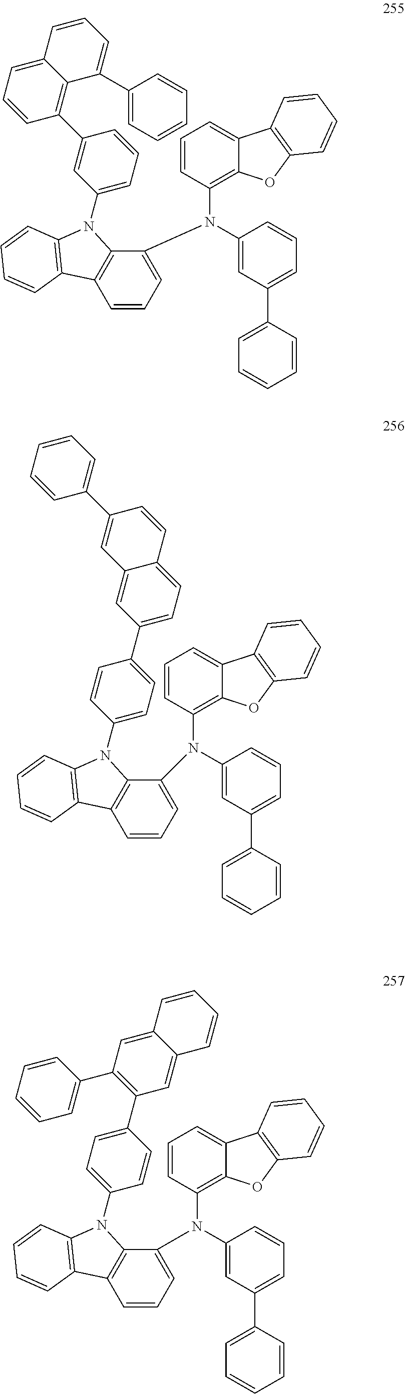 [SMILES: c1ccc(-c2cccc(N(c3cccc4c3oc3ccccc34)c3cccc4c5ccccc5n(-c5cccc(-c6cccc7cccc(-c8ccccc8)c67)c5)c34)c2)cc1 ; c1ccc(-c2cccc(N(c3cccc4c3oc3ccccc34)c3cccc4c5ccccc5n(-c5ccc(-c6ccc7ccc(-c8ccccc8)cc7c6)cc5)c34)c2)cc1 ; c1ccc(-c2cccc(N(c3cccc4c3oc3ccccc34)c3cccc4c5ccccc5n(-c5ccc(-c6cc7ccccc7cc6-c6ccccc6)cc5)c34)c2)cc1] 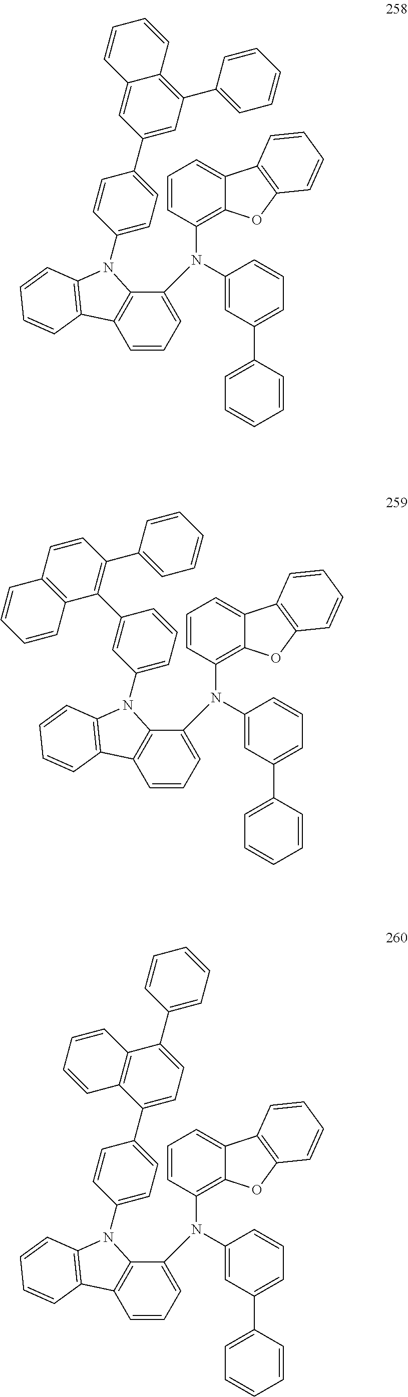 [SMILES: c1ccc(-c2cccc(N(c3cccc4c3oc3ccccc34)c3cccc4c5ccccc5n(-c5ccc(-c6cc(-c7ccccc7)c7ccccc7c6)cc5)c34)c2)cc1 ; c1ccc(-c2cccc(N(c3cccc4c3oc3ccccc34)c3cccc4c5ccccc5n(-c5cccc(-c6c(-c7ccccc7)ccc7ccccc67)c5)c34)c2)cc1 ; c1ccc(-c2cccc(N(c3cccc4c3oc3ccccc34)c3cccc4c5ccccc5n(-c5ccc(-c6ccc(-c7ccccc7)c7ccccc67)cc5)c34)c2)cc1] 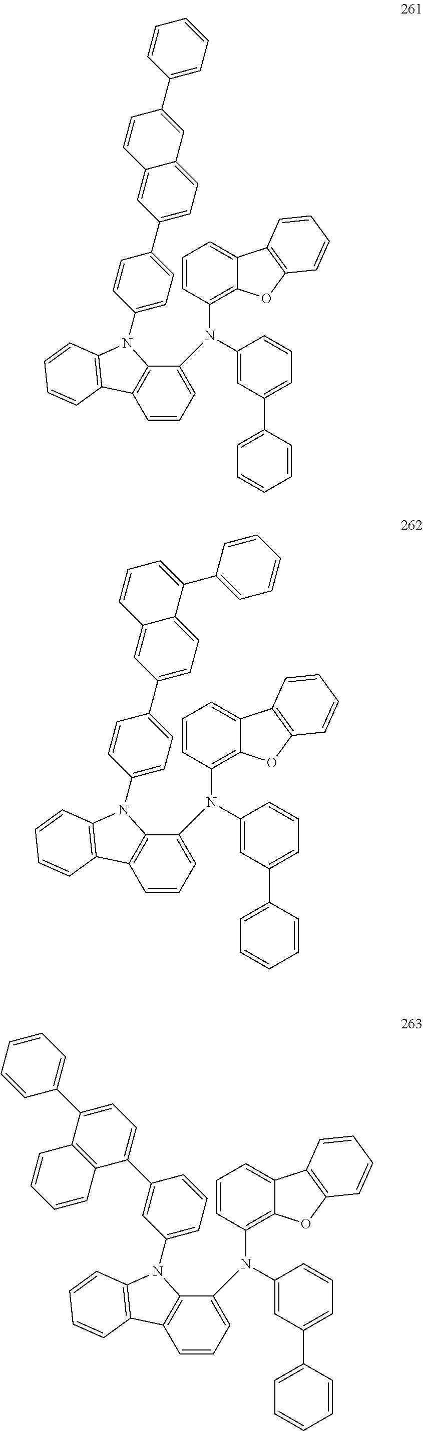 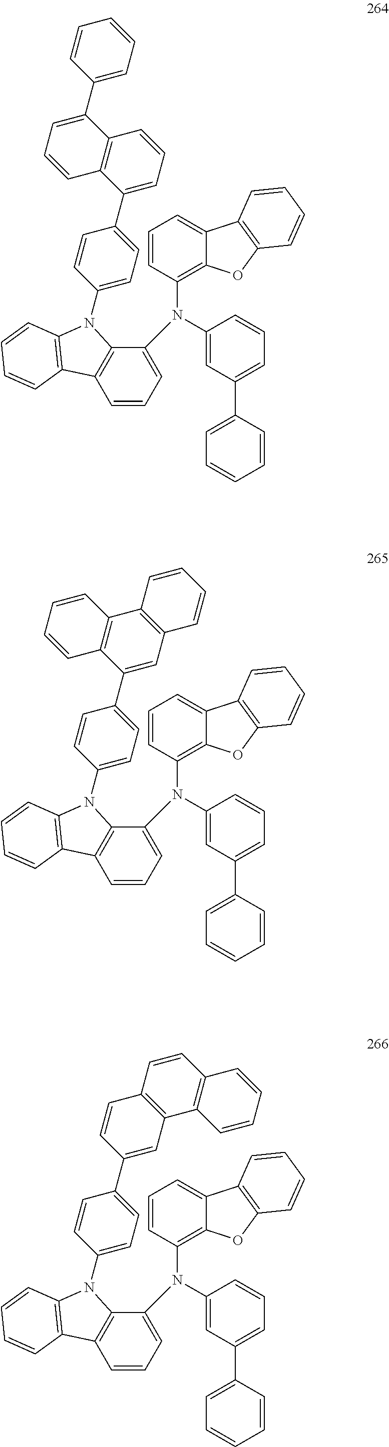 [SMILES: c1ccc(-c2cccc(N(c3cccc4c3oc3ccccc34)c3cccc4c5ccccc5n(-c5ccc(-c6cccc7c(-c8ccccc8)cccc67)cc5)c34)c2)cc1 ; c1ccc(-c2cccc(N(c3cccc4c3oc3ccccc34)c3cccc4c5ccccc5n(-c5ccc(-c6cc7ccccc7c7ccccc67)cc5)c34)c2)cc1 ; c1ccc(-c2cccc(N(c3cccc4c3oc3ccccc34)c3cccc4c5ccccc5n(-c5ccc(-c6ccc7ccc8ccccc8c7c6)cc5)c34)c2)cc1] 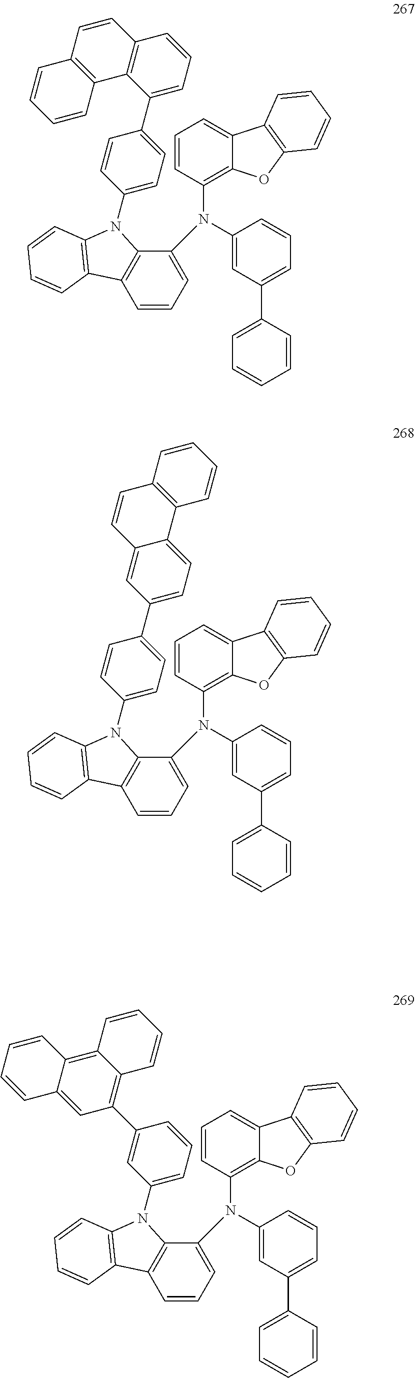 [SMILES: c1ccc(-c2cccc(N(c3cccc4c3oc3ccccc34)c3cccc4c5ccccc5n(-c5ccc(-c6cccc7ccc8ccccc8c67)cc5)c34)c2)cc1 ; c1ccc(-c2cccc(N(c3cccc4c3oc3ccccc34)c3cccc4c5ccccc5n(-c5ccc(-c6ccc7c(ccc8ccccc87)c6)cc5)c34)c2)cc1 ; c1ccc(-c2cccc(N(c3cccc4c3oc3ccccc34)c3cccc4c5ccccc5n(-c5cccc(-c6cc7ccccc7c7ccccc67)c5)c34)c2)cc1] 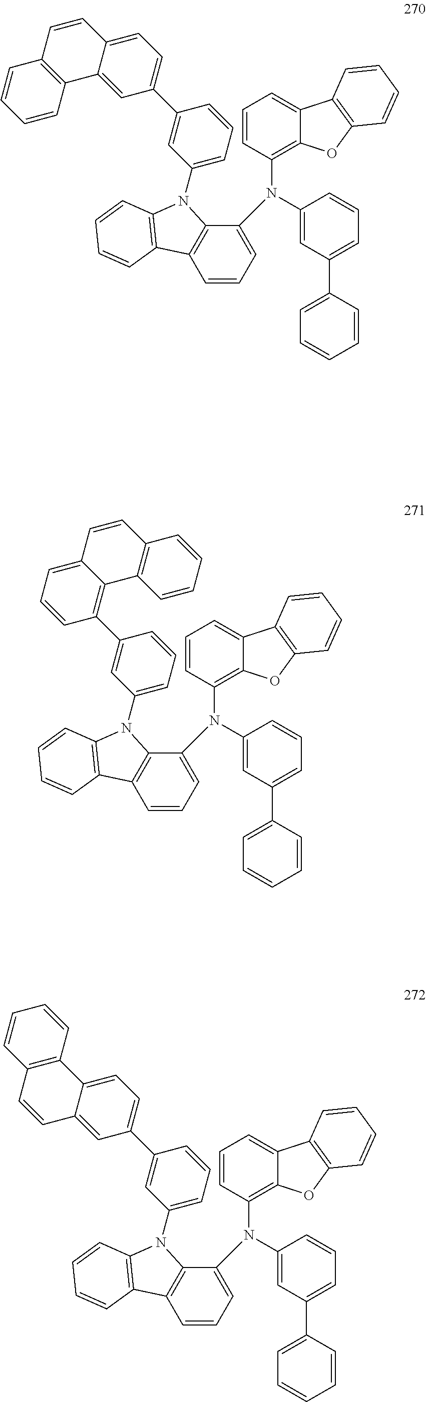 [SMILES: c1ccc(-c2cccc(N(c3cccc4c3oc3ccccc34)c3cccc4c5ccccc5n(-c5cccc(-c6ccc7ccc8ccccc8c7c6)c5)c34)c2)cc1 ; c1ccc(-c2cccc(N(c3cccc4c3oc3ccccc34)c3cccc4c5ccccc5n(-c5cccc(-c6cccc7ccc8ccccc8c67)c5)c34)c2)cc1 ; c1ccc(-c2cccc(N(c3cccc4c3oc3ccccc34)c3cccc4c5ccccc5n(-c5cccc(-c6ccc7c(ccc8ccccc87)c6)c5)c34)c2)cc1] 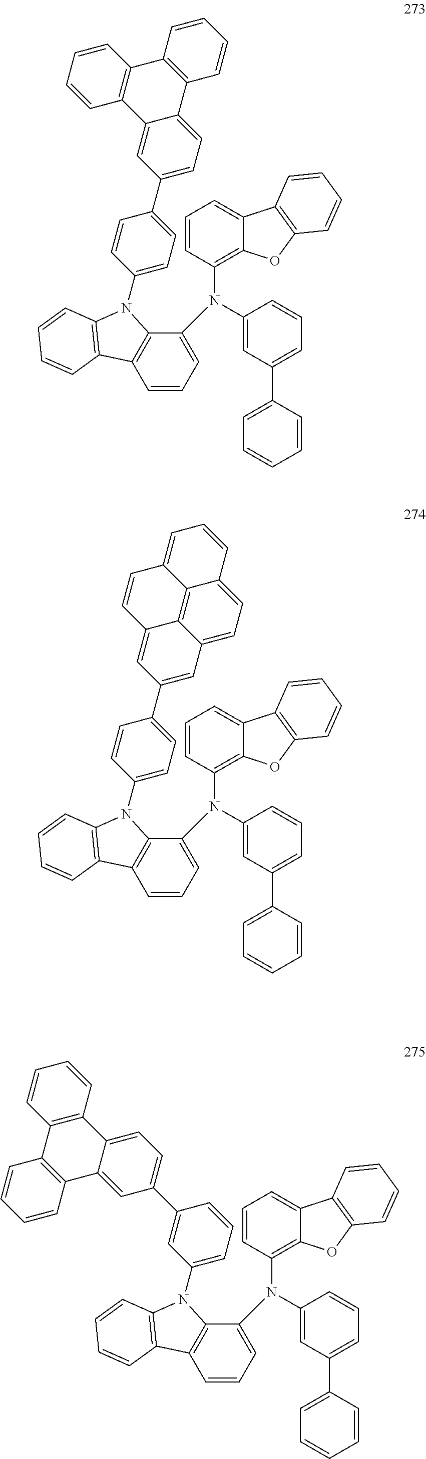 [SMILES: c1ccc(-c2cccc(N(c3cccc4c3oc3ccccc34)c3cccc4c5ccccc5n(-c5ccc(-c6ccc7c8ccccc8c8ccccc8c7c6)cc5)c34)c2)cc1 ; c1ccc(-c2cccc(N(c3cccc4c3oc3ccccc34)c3cccc4c5ccccc5n(-c5ccc(-c6cc7ccc8cccc9ccc(c6)c7c89)cc5)c34)c2)cc1 ; c1ccc(-c2cccc(N(c3cccc4c3oc3ccccc34)c3cccc4c5ccccc5n(-c5cccc(-c6ccc7c8ccccc8c8ccccc8c7c6)c5)c34)c2)cc1] 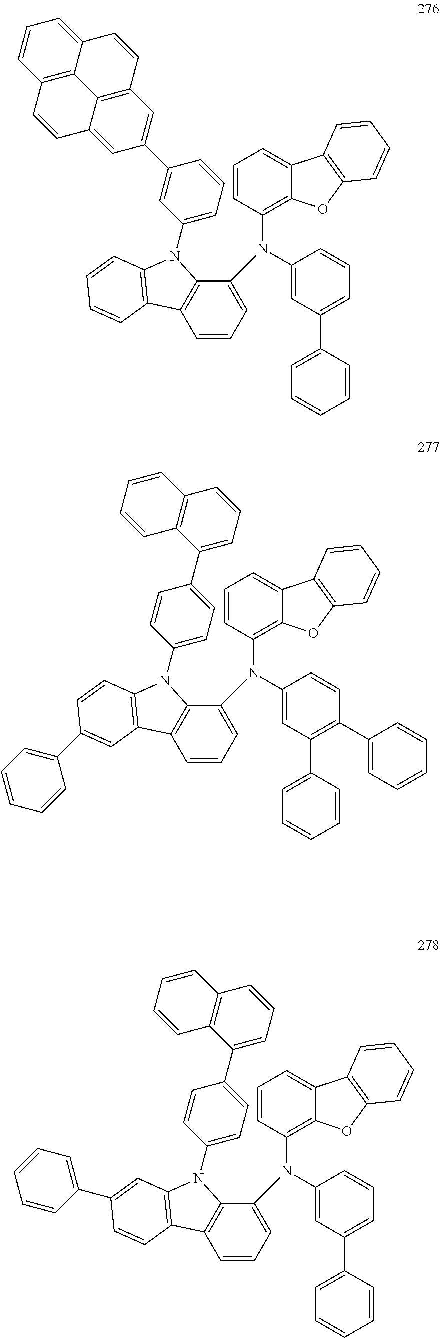 [SMILES: c1ccc(-c2cccc(N(c3cccc4c3oc3ccccc34)c3cccc4c5ccccc5n(-c5cccc(-c6cc7ccc8cccc9ccc(c6)c7c89)c5)c34)c2)cc1 ; c1ccc(-c2ccc3c(c2)c2cccc(N(c4ccc(-c5ccccc5)c(-c5ccccc5)c4)c4cccc5c4oc4ccccc45)c2n3-c2ccc(-c3cccc4ccccc34)cc2)cc1 ; c1ccc(-c2cccc(N(c3cccc4c3oc3ccccc34)c3cccc4c5ccc(-c6ccccc6)cc5n(-c5ccc(-c6cccc7ccccc67)cc5)c34)c2)cc1] 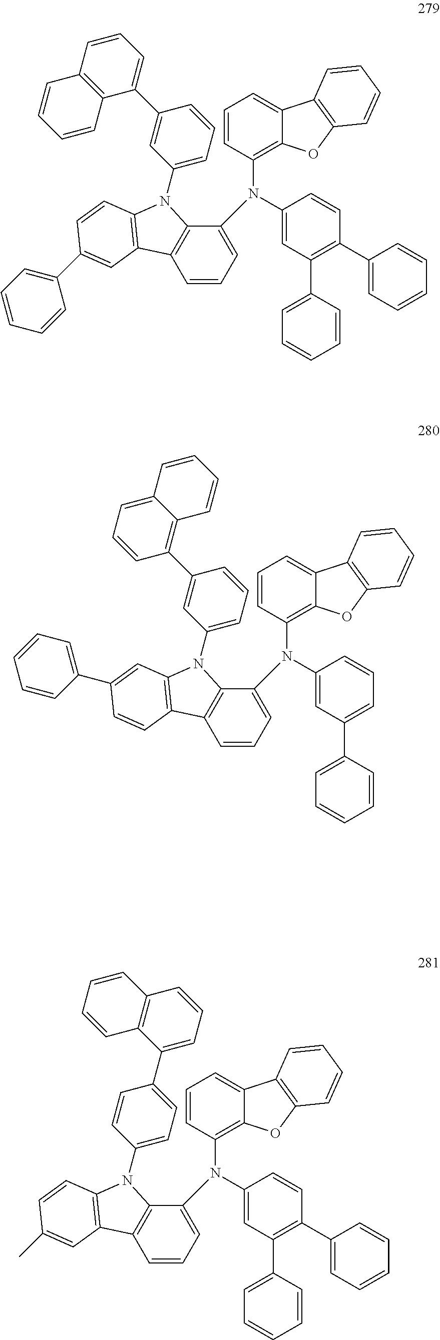 [SMILES: c1ccc(-c2ccc3c(c2)c2cccc(N(c4ccc(-c5ccccc5)c(-c5ccccc5)c4)c4cccc5c4oc4ccccc45)c2n3-c2cccc(-c3cccc4ccccc34)c2)cc1 ; c1ccc(-c2cccc(N(c3cccc4c3oc3ccccc34)c3cccc4c5ccc(-c6ccccc6)cc5n(-c5cccc(-c6cccc7ccccc67)c5)c34)c2)cc1 ; Cc1ccc2c(c1)c1cccc(N(c3ccc(-c4ccccc4)c(-c4ccccc4)c3)c3cccc4c3oc3ccccc34)c1n2-c1ccc(-c2cccc3ccccc23)cc1] 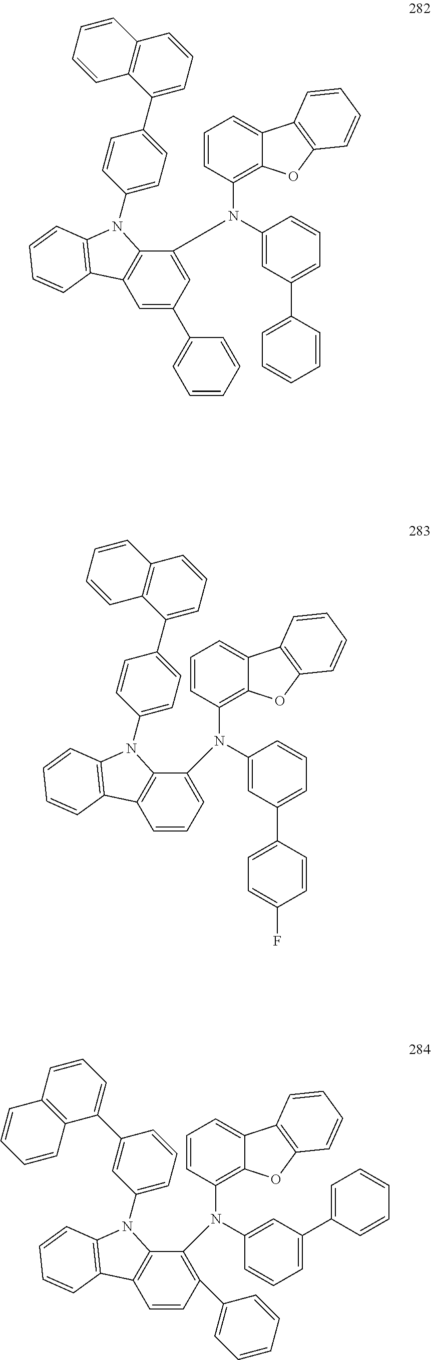 [SMILES: c1ccc(-c2cccc(N(c3cccc4c3oc3ccccc34)c3cc(-c4ccccc4)cc4c5ccccc5n(-c5ccc(-c6cccc7ccccc67)cc5)c34)c2)cc1 ; Fc1ccc(-c2cccc(N(c3cccc4c3oc3ccccc34)c3cccc4c5ccccc5n(-c5ccc(-c6cccc7ccccc67)cc5)c34)c2)cc1 ; c1ccc(-c2cccc(N(c3cccc4c3oc3ccccc34)c3c(-c4ccccc4)ccc4c5ccccc5n(-c5cccc(-c6cccc7ccccc67)c5)c34)c2)cc1] 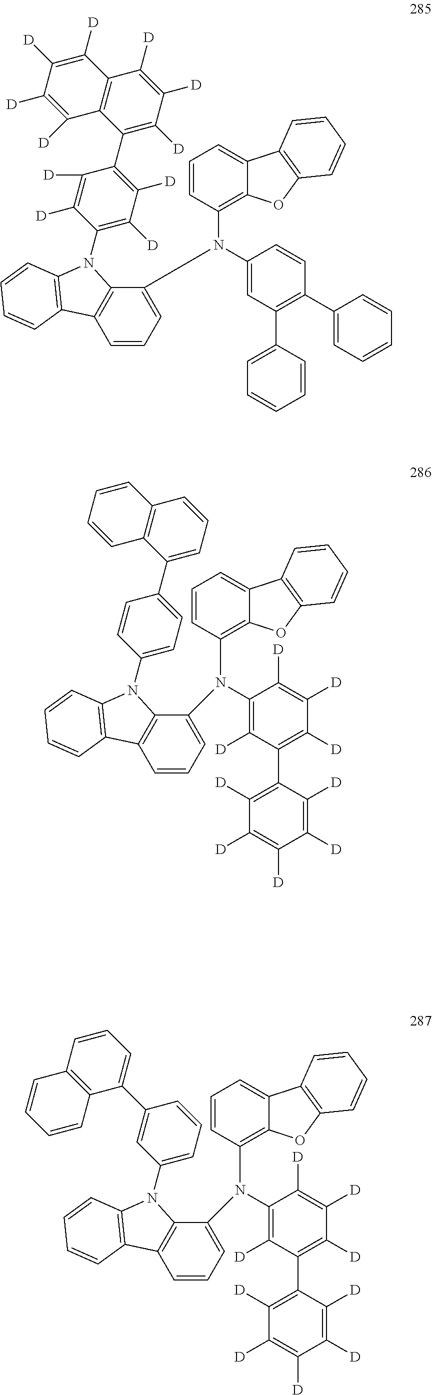 [SMILES: [2H]c1c([2H])c(-n2c3ccccc3c3cccc(N(c4ccc(-c5ccccc5)c(-c5ccccc5)c4)c4cccc5c4oc4ccccc45)c32)c([2H])c([2H])c1-c1c([2H])c([2H])c([2H])c2c([2H])c([2H])c([2H])c([2H])c12 ; [2H]c1c([2H])c([2H])c(-c2c([2H])c([2H])c([2H])c(N(c3cccc4c3oc3ccccc34)c3cccc4c5ccccc5n(-c5ccc(-c6cccc7ccccc67)cc5)c34)c2[2H])c([2H])c1[2H] ; [2H]c1c([2H])c([2H])c(-c2c([2H])c([2H])c([2H])c(N(c3cccc4c3oc3ccccc34)c3cccc4c5ccccc5n(-c5cccc(-c6cccc7ccccc67)c5)c34)c2[2H])c([2H])c1[2H]] 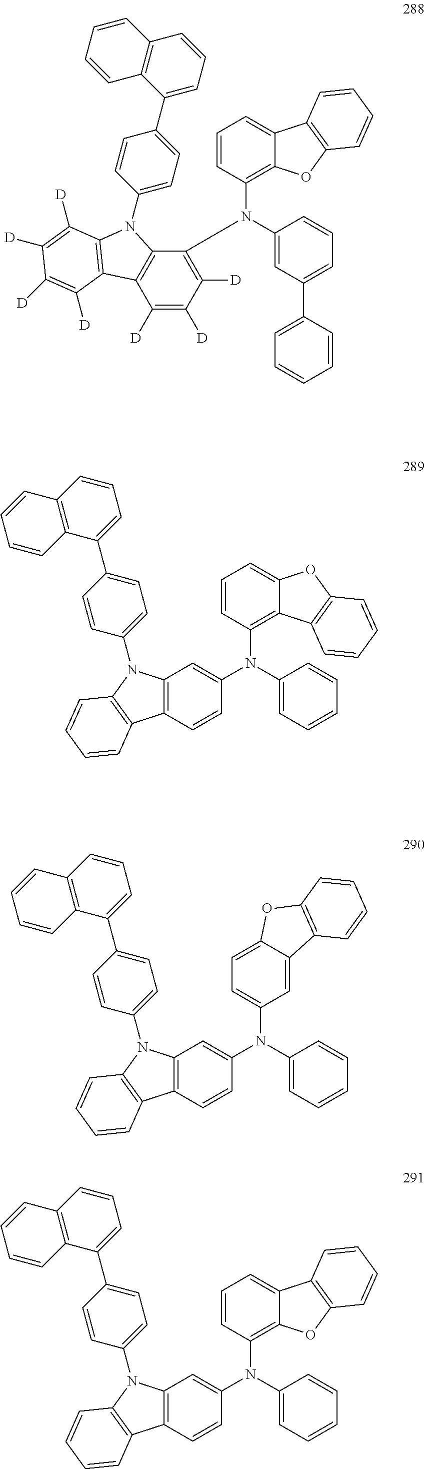 [SMILES: [2H]c1c([2H])c([2H])c2c(c1[2H])c1c([2H])c([2H])c([2H])c(N(c3cccc(-c4ccccc4)c3)c3cccc4c3oc3ccccc34)c1n2-c1ccc(-c2cccc3ccccc23)cc1 ; c1ccc(N(c2ccc3c4ccccc4n(-c4ccc(-c5cccc6ccccc56)cc4)c3c2)c2cccc3oc4ccccc4c23)cc1 ; c1ccc(N(c2ccc3oc4ccccc4c3c2)c2ccc3c4ccccc4n(-c4ccc(-c5cccc6ccccc56)cc4)c3c2)cc1 ; c1ccc(N(c2ccc3c4ccccc4n(-c4ccc(-c5cccc6ccccc56)cc4)c3c2)c2cccc3c2oc2ccccc23)cc1] 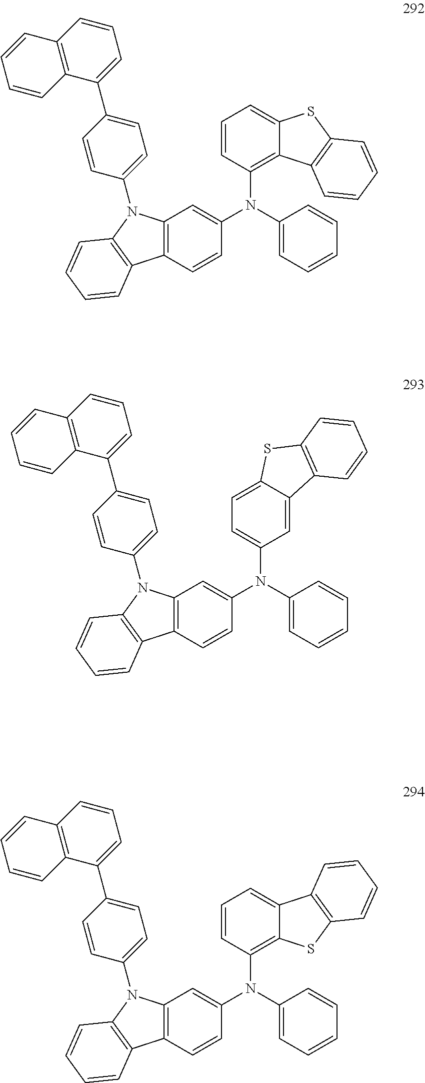 [SMILES: c1ccc(N(c2ccc3c4ccccc4n(-c4ccc(-c5cccc6ccccc56)cc4)c3c2)c2cccc3sc4ccccc4c23)cc1 ; c1ccc(N(c2ccc3sc4ccccc4c3c2)c2ccc3c4ccccc4n(-c4ccc(-c5cccc6ccccc56)cc4)c3c2)cc1 ; c1ccc(N(c2ccc3c4ccccc4n(-c4ccc(-c5cccc6ccccc56)cc4)c3c2)c2cccc3c2sc2ccccc23)cc1] 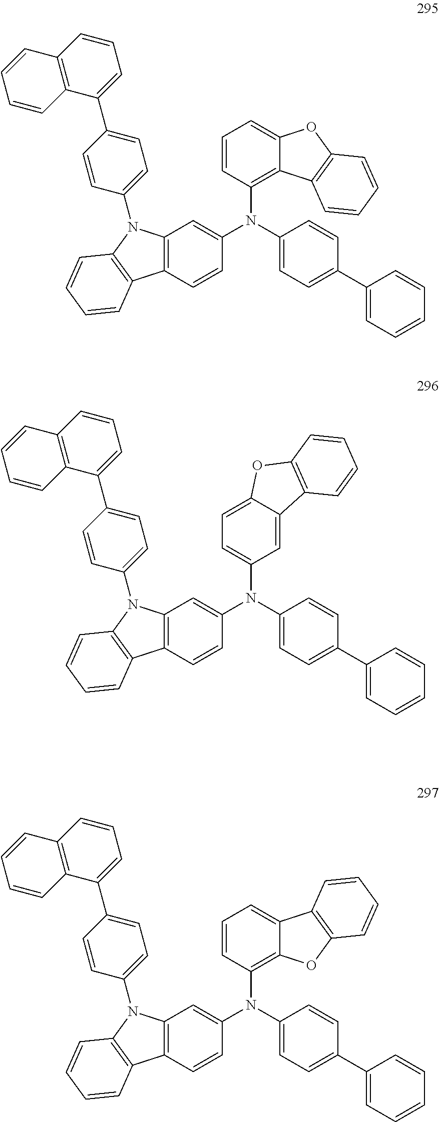 [SMILES: c1ccc(-c2ccc(N(c3ccc4c5ccccc5n(-c5ccc(-c6cccc7ccccc67)cc5)c4c3)c3cccc4oc5ccccc5c34)cc2)cc1 ; c1ccc(-c2ccc(N(c3ccc4oc5ccccc5c4c3)c3ccc4c5ccccc5n(-c5ccc(-c6cccc7ccccc67)cc5)c4c3)cc2)cc1 ; c1ccc(-c2ccc(N(c3ccc4c5ccccc5n(-c5ccc(-c6cccc7ccccc67)cc5)c4c3)c3cccc4c3oc3ccccc34)cc2)cc1] 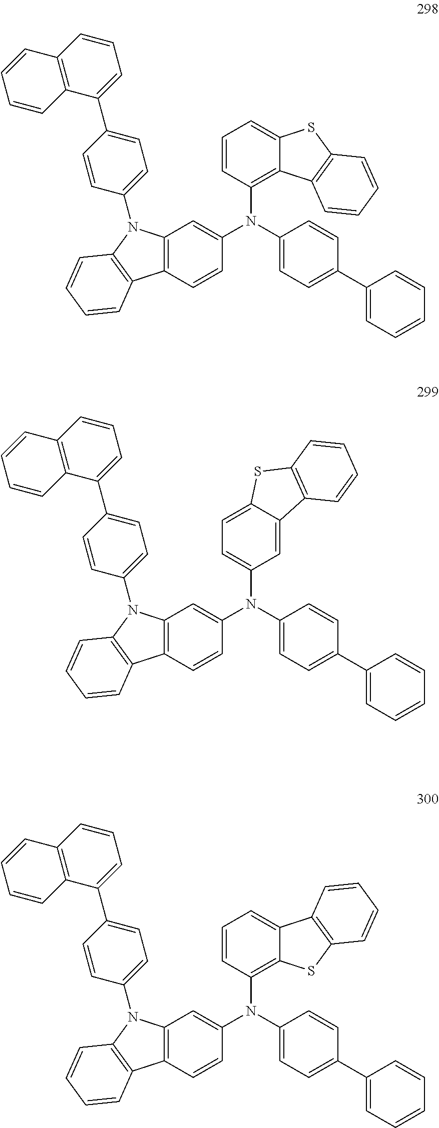 [SMILES: c1ccc(-c2ccc(N(c3ccc4c5ccccc5n(-c5ccc(-c6cccc7ccccc67)cc5)c4c3)c3cccc4sc5ccccc5c34)cc2)cc1 ; c1ccc(-c2ccc(N(c3ccc4sc5ccccc5c4c3)c3ccc4c5ccccc5n(-c5ccc(-c6cccc7ccccc67)cc5)c4c3)cc2)cc1 ; c1ccc(-c2ccc(N(c3ccc4c5ccccc5n(-c5ccc(-c6cccc7ccccc67)cc5)c4c3)c3cccc4c3sc3ccccc34)cc2)cc1] 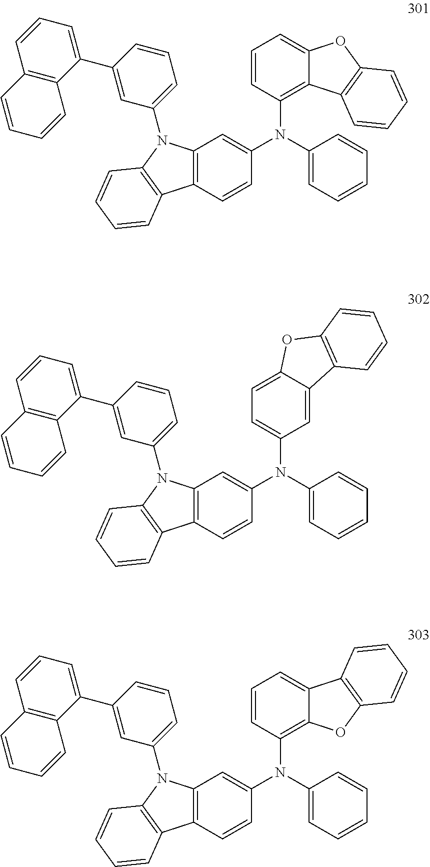 [SMILES: c1ccc(N(c2ccc3c4ccccc4n(-c4cccc(-c5cccc6ccccc56)c4)c3c2)c2cccc3oc4ccccc4c23)cc1 ; c1ccc(N(c2ccc3oc4ccccc4c3c2)c2ccc3c4ccccc4n(-c4cccc(-c5cccc6ccccc56)c4)c3c2)cc1 ; c1ccc(N(c2ccc3c4ccccc4n(-c4cccc(-c5cccc6ccccc56)c4)c3c2)c2cccc3c2oc2ccccc23)cc1] 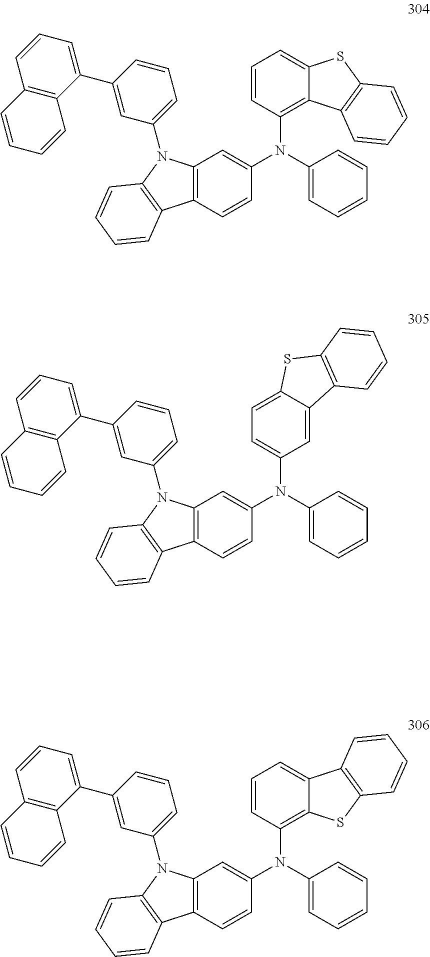 [SMILES: c1ccc(N(c2ccc3c4ccccc4n(-c4cccc(-c5cccc6ccccc56)c4)c3c2)c2cccc3sc4ccccc4c23)cc1 ; c1ccc(N(c2ccc3sc4ccccc4c3c2)c2ccc3c4ccccc4n(-c4cccc(-c5cccc6ccccc56)c4)c3c2)cc1 ; c1ccc(N(c2ccc3c4ccccc4n(-c4cccc(-c5cccc6ccccc56)c4)c3c2)c2cccc3c2sc2ccccc23)cc1] 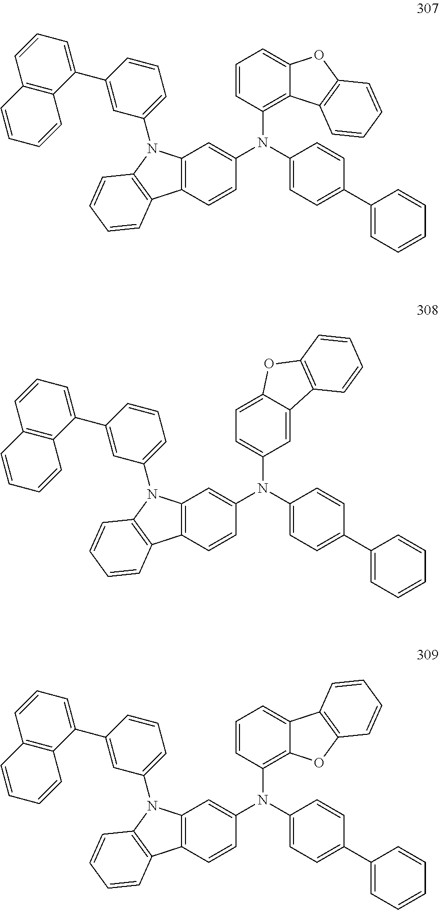 [SMILES: c1ccc(-c2ccc(N(c3ccc4c5ccccc5n(-c5cccc(-c6cccc7ccccc67)c5)c4c3)c3cccc4oc5ccccc5c34)cc2)cc1 ; c1ccc(-c2ccc(N(c3ccc4oc5ccccc5c4c3)c3ccc4c5ccccc5n(-c5cccc(-c6cccc7ccccc67)c5)c4c3)cc2)cc1 ; c1ccc(-c2ccc(N(c3ccc4c5ccccc5n(-c5cccc(-c6cccc7ccccc67)c5)c4c3)c3cccc4c3oc3ccccc34)cc2)cc1] 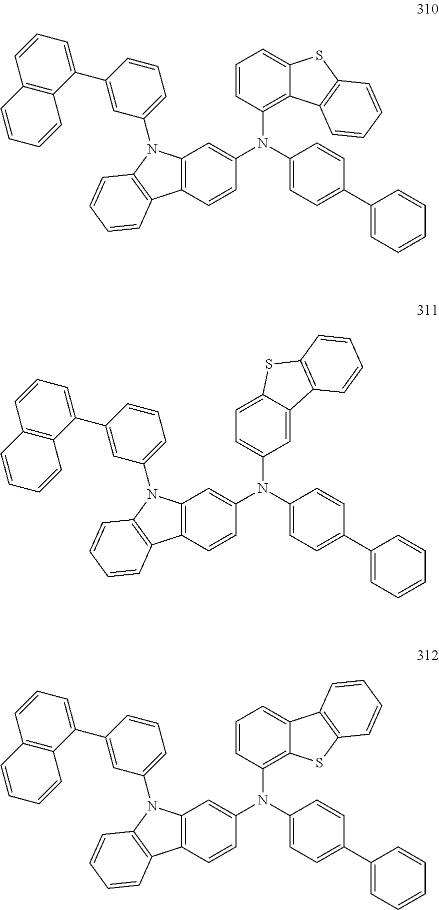 [SMILES: c1ccc(-c2ccc(N(c3ccc4c5ccccc5n(-c5cccc(-c6cccc7ccccc67)c5)c4c3)c3cccc4sc5ccccc5c34)cc2)cc1 ; c1ccc(-c2ccc(N(c3ccc4sc5ccccc5c4c3)c3ccc4c5ccccc5n(-c5cccc(-c6cccc7ccccc67)c5)c4c3)cc2)cc1 ; c1ccc(-c2ccc(N(c3ccc4c5ccccc5n(-c5cccc(-c6cccc7ccccc67)c5)c4c3)c3cccc4c3sc3ccccc34)cc2)cc1] 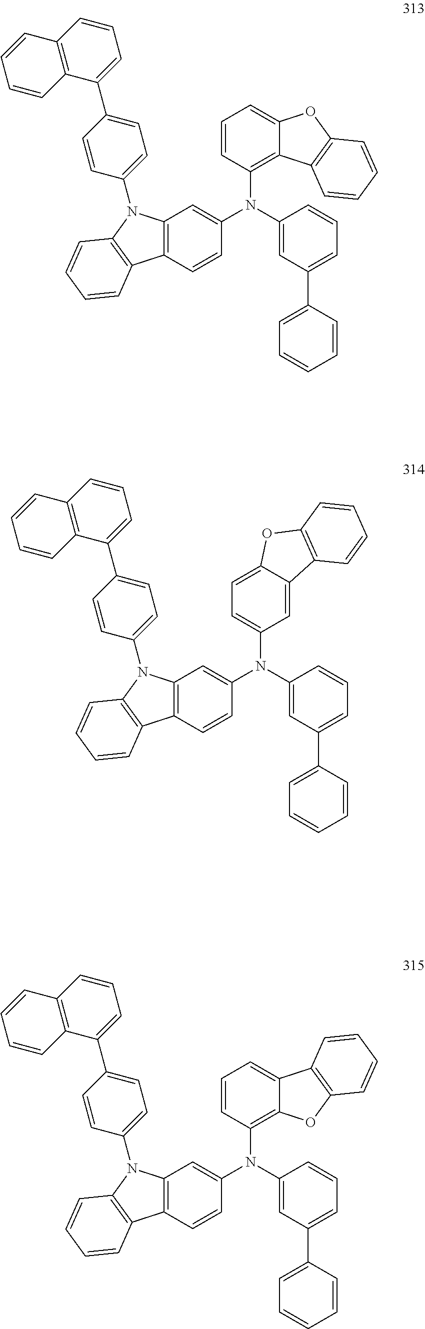 [SMILES: c1ccc(-c2cccc(N(c3ccc4c5ccccc5n(-c5ccc(-c6cccc7ccccc67)cc5)c4c3)c3cccc4oc5ccccc5c34)c2)cc1 ; c1ccc(-c2cccc(N(c3ccc4oc5ccccc5c4c3)c3ccc4c5ccccc5n(-c5ccc(-c6cccc7ccccc67)cc5)c4c3)c2)cc1 ; c1ccc(-c2cccc(N(c3ccc4c5ccccc5n(-c5ccc(-c6cccc7ccccc67)cc5)c4c3)c3cccc4c3oc3ccccc34)c2)cc1] 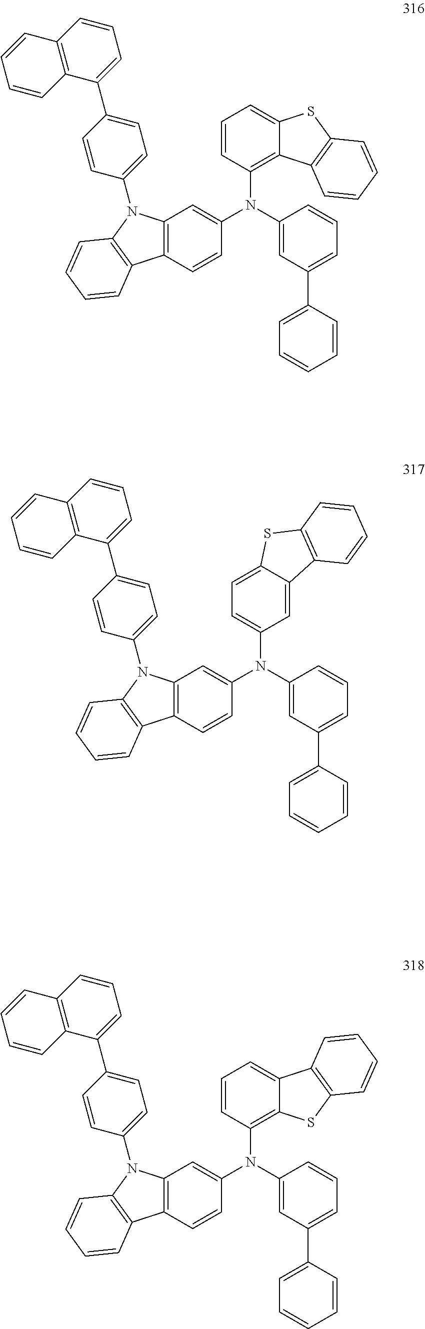 [SMILES: c1ccc(-c2cccc(N(c3ccc4c5ccccc5n(-c5ccc(-c6cccc7ccccc67)cc5)c4c3)c3cccc4sc5ccccc5c34)c2)cc1 ; c1ccc(-c2cccc(N(c3ccc4sc5ccccc5c4c3)c3ccc4c5ccccc5n(-c5ccc(-c6cccc7ccccc67)cc5)c4c3)c2)cc1 ; c1ccc(-c2cccc(N(c3ccc4c5ccccc5n(-c5ccc(-c6cccc7ccccc67)cc5)c4c3)c3cccc4c3sc3ccccc34)c2)cc1] 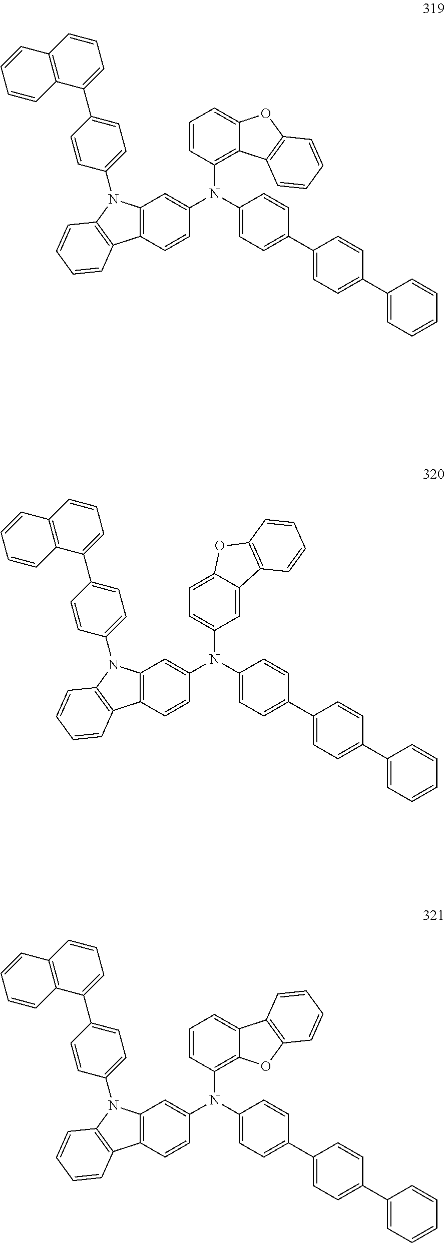 [SMILES: c1ccc(-c2ccc(-c3ccc(N(c4ccc5c6ccccc6n(-c6ccc(-c7cccc8ccccc78)cc6)c5c4)c4cccc5oc6ccccc6c45)cc3)cc2)cc1 ; c1ccc(-c2ccc(-c3ccc(N(c4ccc5oc6ccccc6c5c4)c4ccc5c6ccccc6n(-c6ccc(-c7cccc8ccccc78)cc6)c5c4)cc3)cc2)cc1 ; c1ccc(-c2ccc(-c3ccc(N(c4ccc5c6ccccc6n(-c6ccc(-c7cccc8ccccc78)cc6)c5c4)c4cccc5c4oc4ccccc45)cc3)cc2)cc1] 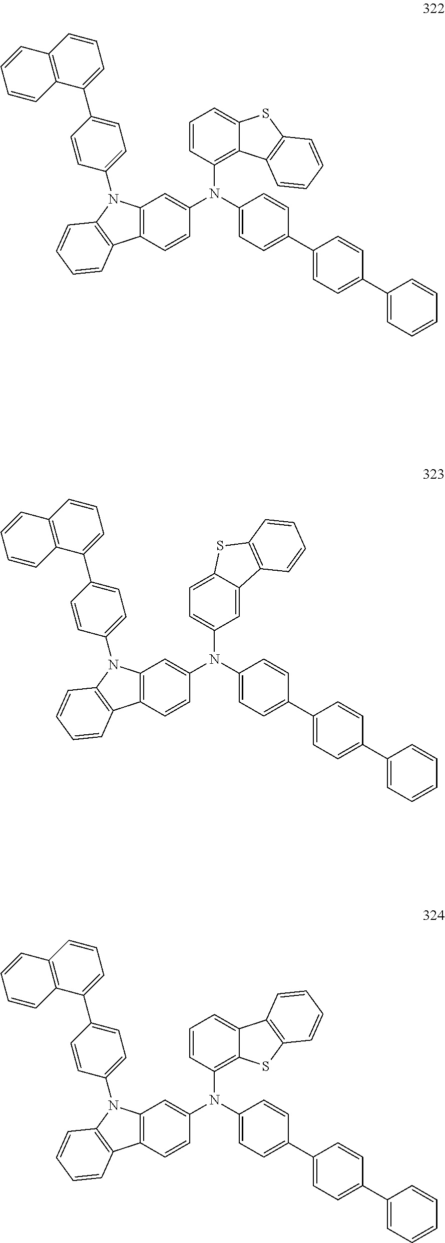 [SMILES: c1ccc(-c2ccc(-c3ccc(N(c4ccc5c6ccccc6n(-c6ccc(-c7cccc8ccccc78)cc6)c5c4)c4cccc5sc6ccccc6c45)cc3)cc2)cc1 ; c1ccc(-c2ccc(-c3ccc(N(c4ccc5sc6ccccc6c5c4)c4ccc5c6ccccc6n(-c6ccc(-c7cccc8ccccc78)cc6)c5c4)cc3)cc2)cc1 ; c1ccc(-c2ccc(-c3ccc(N(c4ccc5c6ccccc6n(-c6ccc(-c7cccc8ccccc78)cc6)c5c4)c4cccc5c4sc4ccccc45)cc3)cc2)cc1] 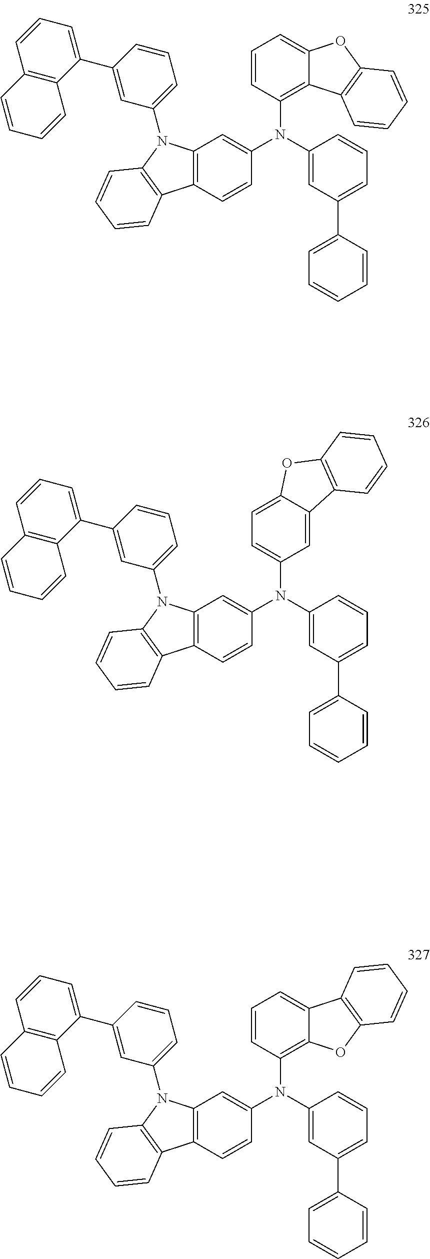 [SMILES: c1ccc(-c2cccc(N(c3ccc4c5ccccc5n(-c5cccc(-c6cccc7ccccc67)c5)c4c3)c3cccc4oc5ccccc5c34)c2)cc1 ; c1ccc(-c2cccc(N(c3ccc4oc5ccccc5c4c3)c3ccc4c5ccccc5n(-c5cccc(-c6cccc7ccccc67)c5)c4c3)c2)cc1 ; c1ccc(-c2cccc(N(c3ccc4c5ccccc5n(-c5cccc(-c6cccc7ccccc67)c5)c4c3)c3cccc4c3oc3ccccc34)c2)cc1] 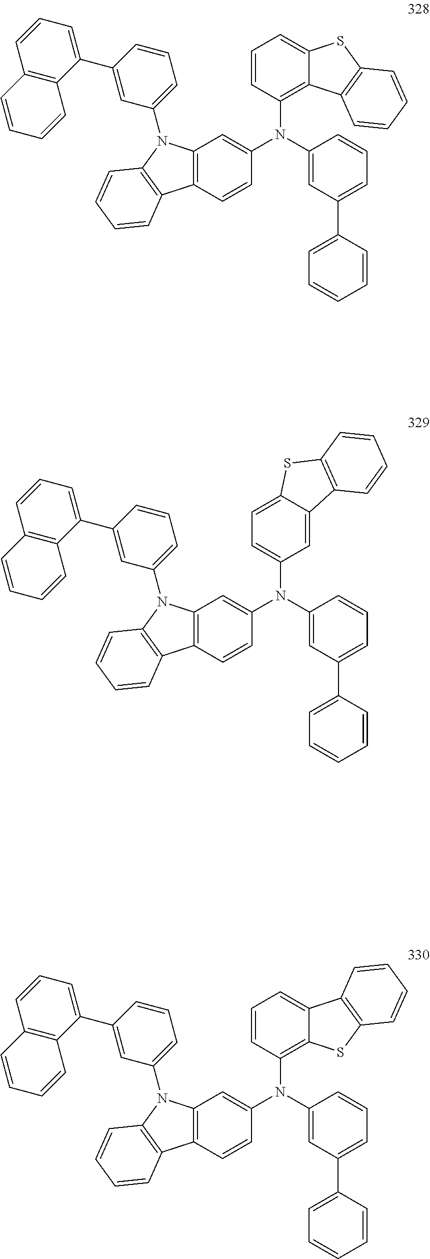 [SMILES: c1ccc(-c2cccc(N(c3ccc4c5ccccc5n(-c5cccc(-c6cccc7ccccc67)c5)c4c3)c3cccc4sc5ccccc5c34)c2)cc1 ; c1ccc(-c2cccc(N(c3ccc4sc5ccccc5c4c3)c3ccc4c5ccccc5n(-c5cccc(-c6cccc7ccccc67)c5)c4c3)c2)cc1 ; c1ccc(-c2cccc(N(c3ccc4c5ccccc5n(-c5cccc(-c6cccc7ccccc67)c5)c4c3)c3cccc4c3sc3ccccc34)c2)cc1] 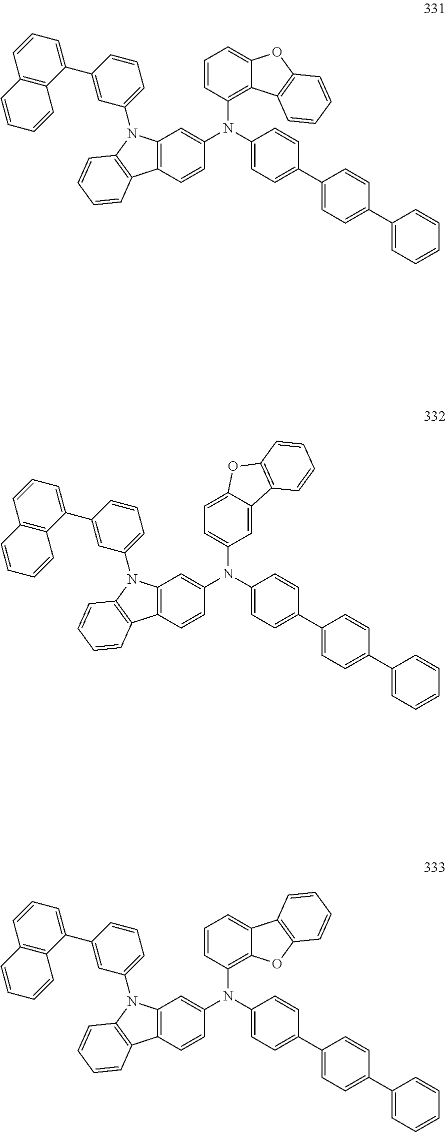 [SMILES: c1ccc(-c2ccc(-c3ccc(N(c4ccc5c6ccccc6n(-c6cccc(-c7cccc8ccccc78)c6)c5c4)c4cccc5oc6ccccc6c45)cc3)cc2)cc1 ; c1ccc(-c2ccc(-c3ccc(N(c4ccc5oc6ccccc6c5c4)c4ccc5c6ccccc6n(-c6cccc(-c7cccc8ccccc78)c6)c5c4)cc3)cc2)cc1 ; c1ccc(-c2ccc(-c3ccc(N(c4ccc5c6ccccc6n(-c6cccc(-c7cccc8ccccc78)c6)c5c4)c4cccc5c4oc4ccccc45)cc3)cc2)cc1] 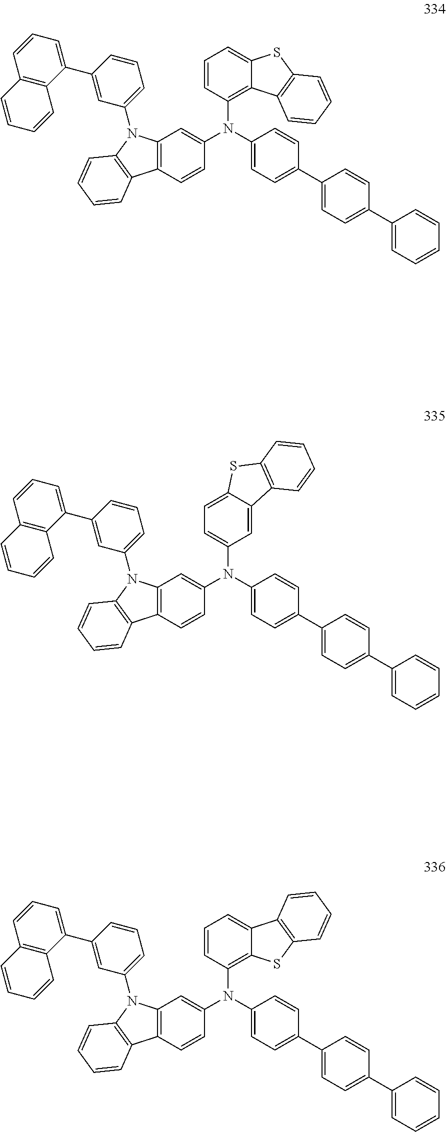 [SMILES: c1ccc(-c2ccc(-c3ccc(N(c4ccc5c6ccccc6n(-c6cccc(-c7cccc8ccccc78)c6)c5c4)c4cccc5sc6ccccc6c45)cc3)cc2)cc1 ; c1ccc(-c2ccc(-c3ccc(N(c4ccc5sc6ccccc6c5c4)c4ccc5c6ccccc6n(-c6cccc(-c7cccc8ccccc78)c6)c5c4)cc3)cc2)cc1 ; c1ccc(-c2ccc(-c3ccc(N(c4ccc5c6ccccc6n(-c6cccc(-c7cccc8ccccc78)c6)c5c4)c4cccc5c4sc4ccccc45)cc3)cc2)cc1] 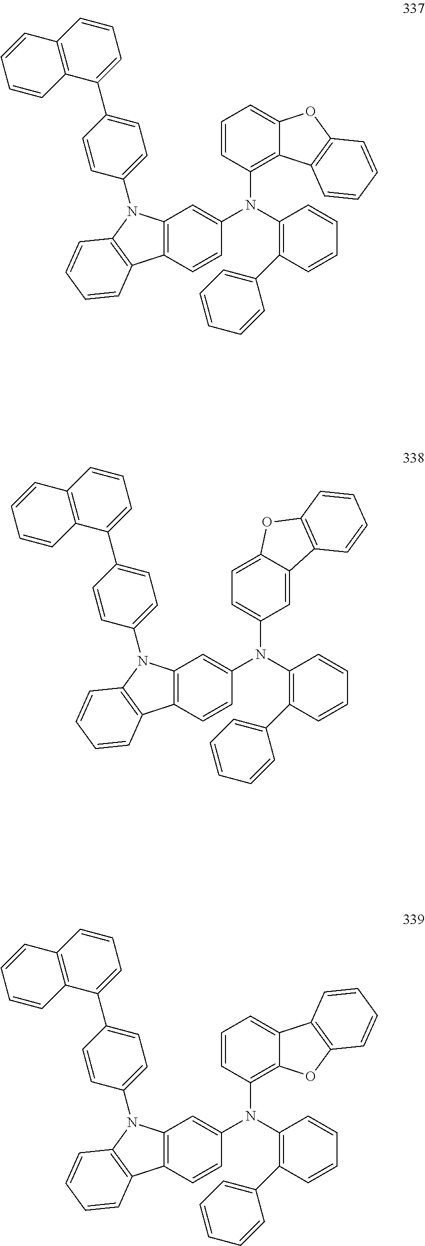 [SMILES: c1ccc(-c2ccccc2N(c2ccc3c4ccccc4n(-c4ccc(-c5cccc6ccccc56)cc4)c3c2)c2cccc3oc4ccccc4c23)cc1 ; c1ccc(-c2ccccc2N(c2ccc3oc4ccccc4c3c2)c2ccc3c4ccccc4n(-c4ccc(-c5cccc6ccccc56)cc4)c3c2)cc1 ; c1ccc(-c2ccccc2N(c2ccc3c4ccccc4n(-c4ccc(-c5cccc6ccccc56)cc4)c3c2)c2cccc3c2oc2ccccc23)cc1] 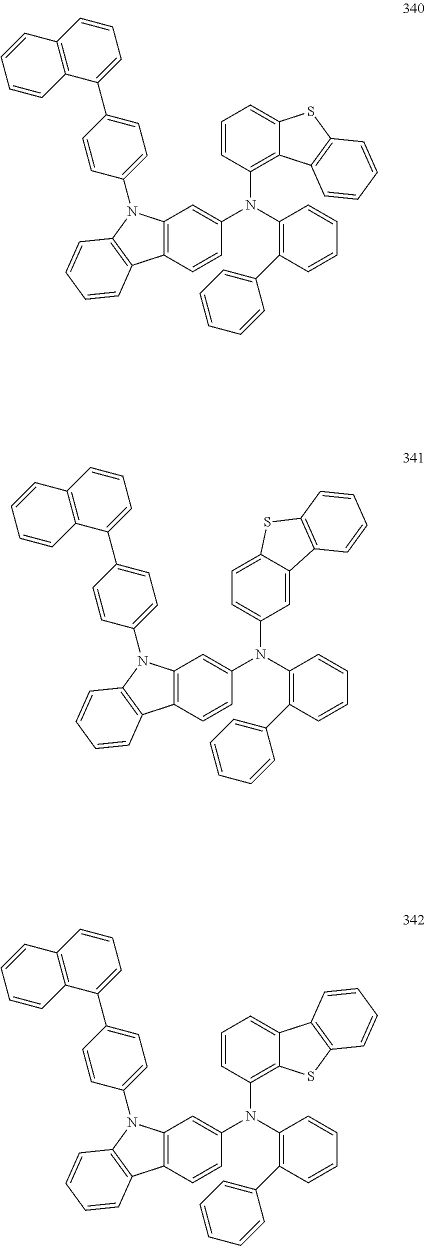 [SMILES: c1ccc(-c2ccccc2N(c2ccc3c4ccccc4n(-c4ccc(-c5cccc6ccccc56)cc4)c3c2)c2cccc3sc4ccccc4c23)cc1 ; c1ccc(-c2ccccc2N(c2ccc3sc4ccccc4c3c2)c2ccc3c4ccccc4n(-c4ccc(-c5cccc6ccccc56)cc4)c3c2)cc1 ; c1ccc(-c2ccccc2N(c2ccc3c4ccccc4n(-c4ccc(-c5cccc6ccccc56)cc4)c3c2)c2cccc3c2sc2ccccc23)cc1] 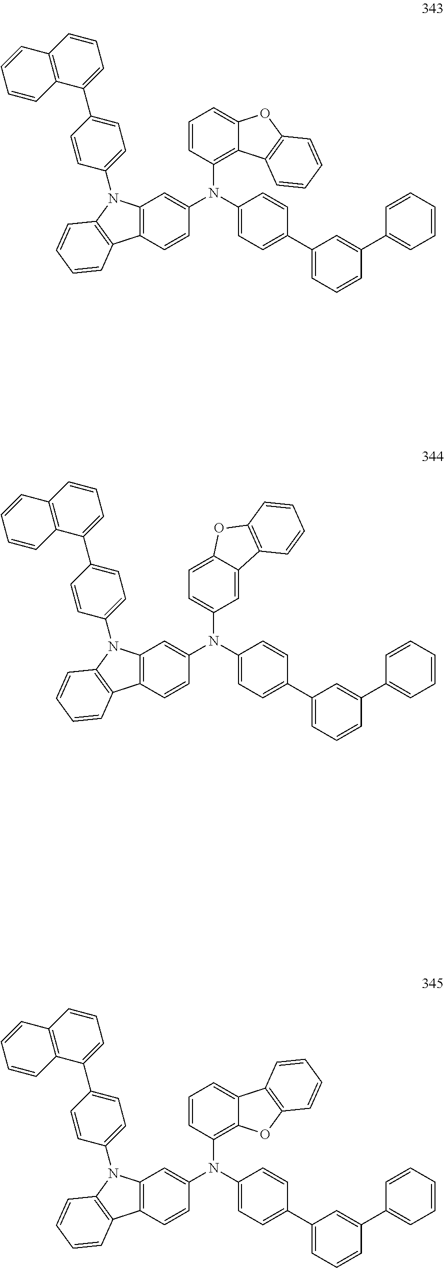 [SMILES: c1ccc(-c2cccc(-c3ccc(N(c4ccc5c6ccccc6n(-c6ccc(-c7cccc8ccccc78)cc6)c5c4)c4cccc5oc6ccccc6c45)cc3)c2)cc1 ; c1ccc(-c2cccc(-c3ccc(N(c4ccc5oc6ccccc6c5c4)c4ccc5c6ccccc6n(-c6ccc(-c7cccc8ccccc78)cc6)c5c4)cc3)c2)cc1 ; c1ccc(-c2cccc(-c3ccc(N(c4ccc5c6ccccc6n(-c6ccc(-c7cccc8ccccc78)cc6)c5c4)c4cccc5c4oc4ccccc45)cc3)c2)cc1] 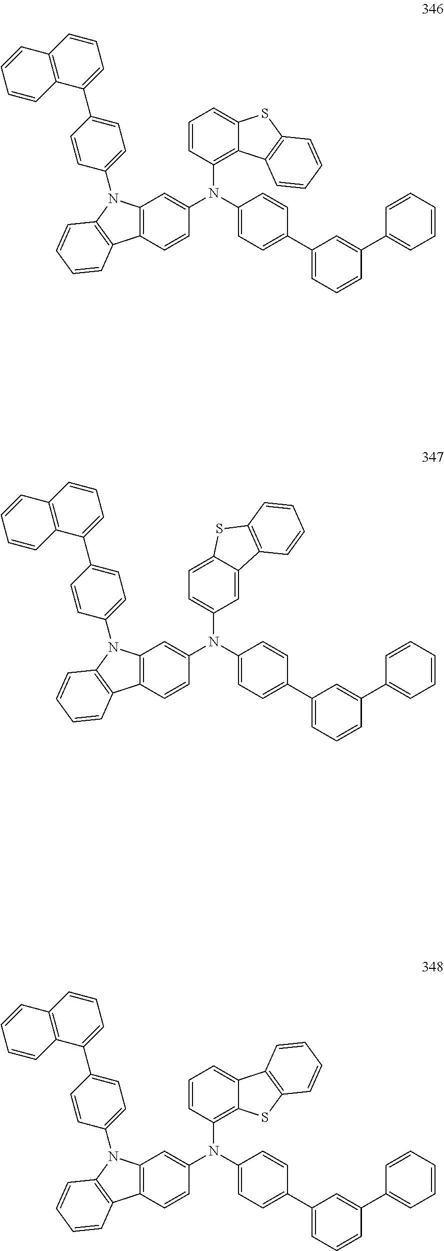 [SMILES: c1ccc(-c2cccc(-c3ccc(N(c4ccc5c6ccccc6n(-c6ccc(-c7cccc8ccccc78)cc6)c5c4)c4cccc5sc6ccccc6c45)cc3)c2)cc1 ; c1ccc(-c2cccc(-c3ccc(N(c4ccc5sc6ccccc6c5c4)c4ccc5c6ccccc6n(-c6ccc(-c7cccc8ccccc78)cc6)c5c4)cc3)c2)cc1 ; c1ccc(-c2cccc(-c3ccc(N(c4ccc5c6ccccc6n(-c6ccc(-c7cccc8ccccc78)cc6)c5c4)c4cccc5c4sc4ccccc45)cc3)c2)cc1] 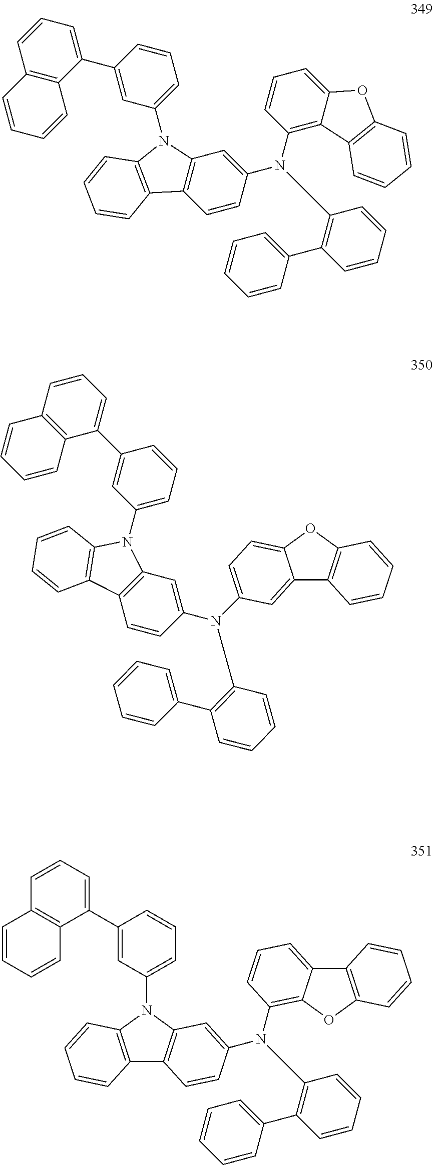 [SMILES: c1ccc(-c2ccccc2N(c2ccc3c4ccccc4n(-c4cccc(-c5cccc6ccccc56)c4)c3c2)c2cccc3oc4ccccc4c23)cc1 ; c1ccc(-c2ccccc2N(c2ccc3oc4ccccc4c3c2)c2ccc3c4ccccc4n(-c4cccc(-c5cccc6ccccc56)c4)c3c2)cc1 ; c1ccc(-c2ccccc2N(c2ccc3c4ccccc4n(-c4cccc(-c5cccc6ccccc56)c4)c3c2)c2cccc3c2oc2ccccc23)cc1] 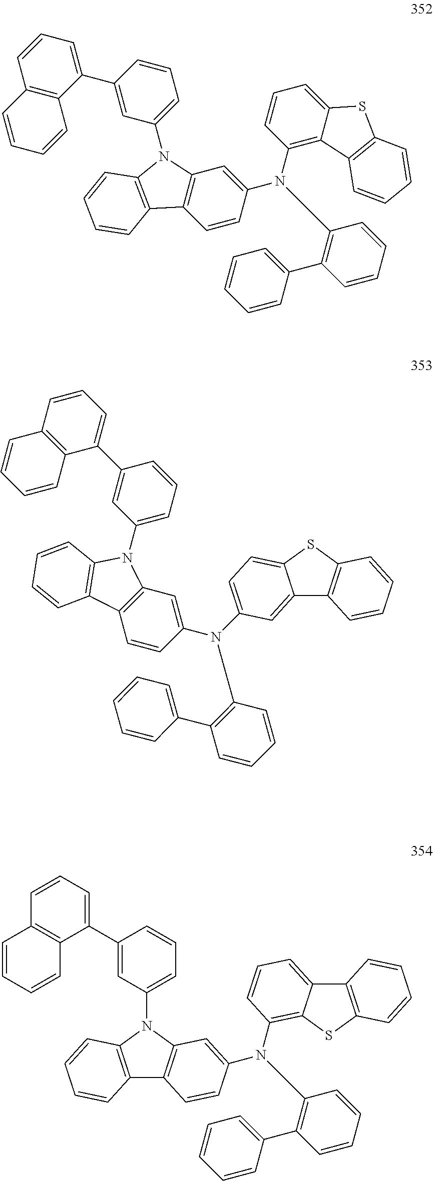 [SMILES: c1ccc(-c2ccccc2N(c2ccc3c4ccccc4n(-c4cccc(-c5cccc6ccccc56)c4)c3c2)c2cccc3sc4ccccc4c23)cc1 ; c1ccc(-c2ccccc2N(c2ccc3sc4ccccc4c3c2)c2ccc3c4ccccc4n(-c4cccc(-c5cccc6ccccc56)c4)c3c2)cc1 ; c1ccc(-c2ccccc2N(c2ccc3c4ccccc4n(-c4cccc(-c5cccc6ccccc56)c4)c3c2)c2cccc3c2sc2ccccc23)cc1] 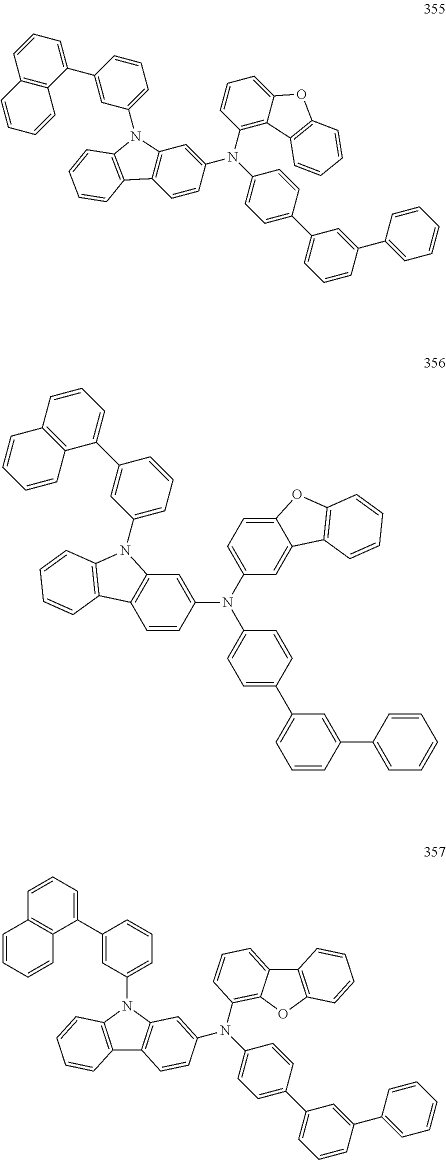 [SMILES: c1ccc(-c2cccc(-c3ccc(N(c4ccc5c6ccccc6n(-c6cccc(-c7cccc8ccccc78)c6)c5c4)c4cccc5oc6ccccc6c45)cc3)c2)cc1 ; c1ccc(-c2cccc(-c3ccc(N(c4ccc5oc6ccccc6c5c4)c4ccc5c6ccccc6n(-c6cccc(-c7cccc8ccccc78)c6)c5c4)cc3)c2)cc1 ; c1ccc(-c2cccc(-c3ccc(N(c4ccc5c6ccccc6n(-c6cccc(-c7cccc8ccccc78)c6)c5c4)c4cccc5c4oc4ccccc45)cc3)c2)cc1] 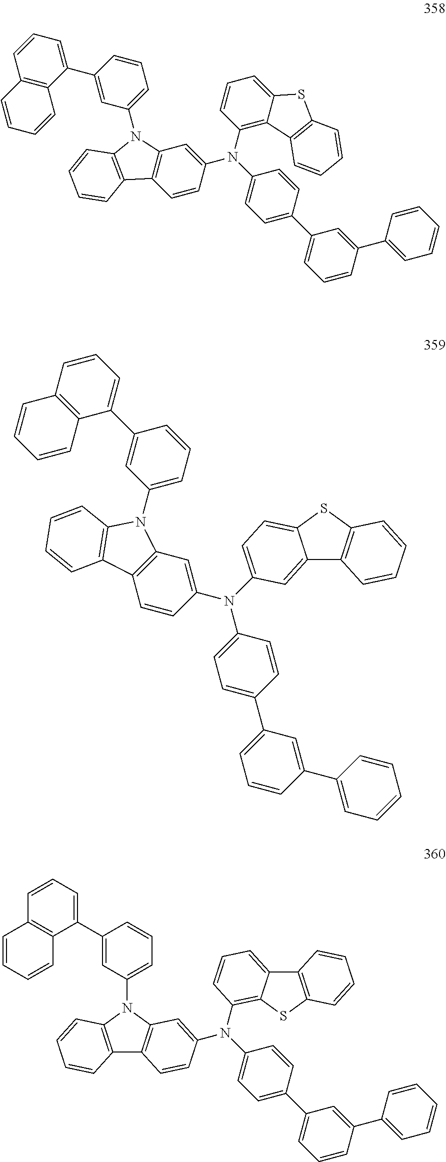 [SMILES: c1ccc(-c2cccc(-c3ccc(N(c4ccc5c6ccccc6n(-c6cccc(-c7cccc8ccccc78)c6)c5c4)c4cccc5sc6ccccc6c45)cc3)c2)cc1 ; c1ccc(-c2cccc(-c3ccc(N(c4ccc5sc6ccccc6c5c4)c4ccc5c6ccccc6n(-c6cccc(-c7cccc8ccccc78)c6)c5c4)cc3)c2)cc1 ; c1ccc(-c2cccc(-c3ccc(N(c4ccc5c6ccccc6n(-c6cccc(-c7cccc8ccccc78)c6)c5c4)c4cccc5c4sc4ccccc45)cc3)c2)cc1] 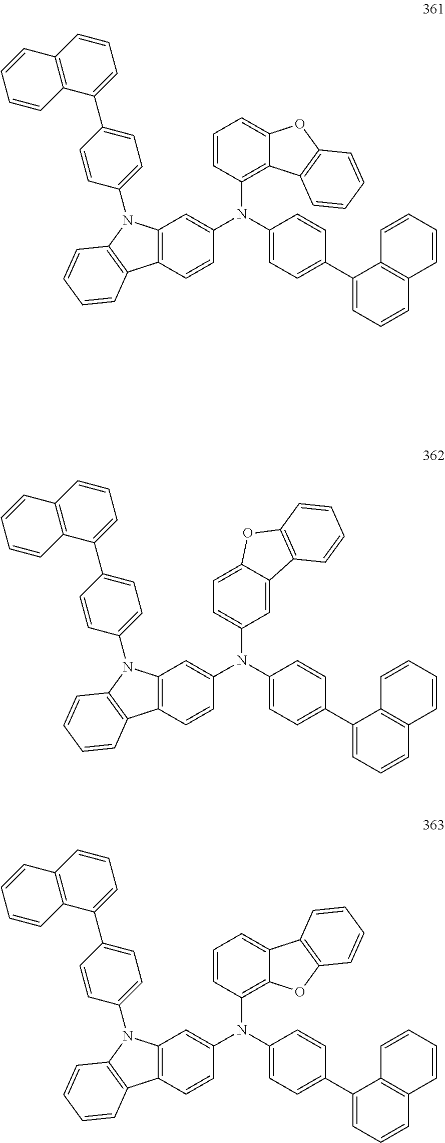 [SMILES: c1ccc2c(-c3ccc(N(c4ccc5c6ccccc6n(-c6ccc(-c7cccc8ccccc78)cc6)c5c4)c4cccc5oc6ccccc6c45)cc3)cccc2c1 ; c1ccc2c(-c3ccc(N(c4ccc5oc6ccccc6c5c4)c4ccc5c6ccccc6n(-c6ccc(-c7cccc8ccccc78)cc6)c5c4)cc3)cccc2c1 ; c1ccc2c(-c3ccc(N(c4ccc5c6ccccc6n(-c6ccc(-c7cccc8ccccc78)cc6)c5c4)c4cccc5c4oc4ccccc45)cc3)cccc2c1] 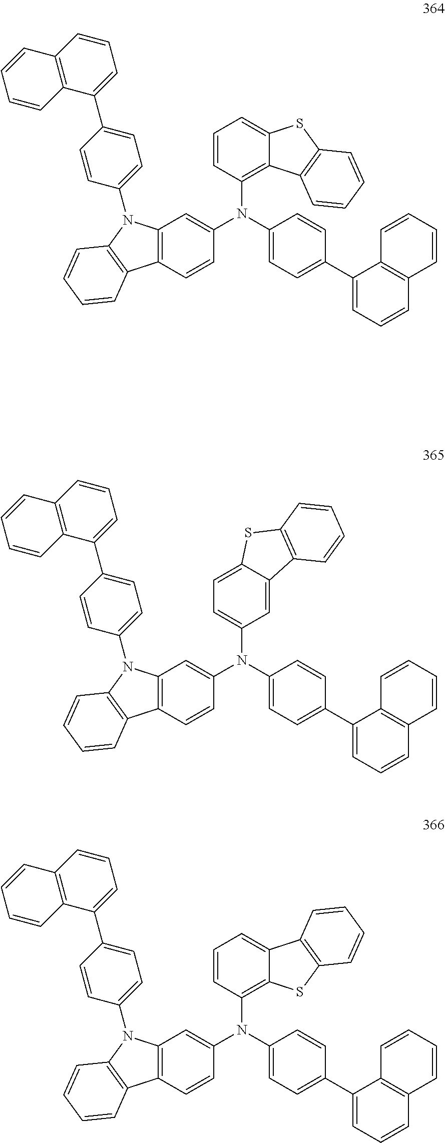 [SMILES: c1ccc2c(-c3ccc(N(c4ccc5c6ccccc6n(-c6ccc(-c7cccc8ccccc78)cc6)c5c4)c4cccc5sc6ccccc6c45)cc3)cccc2c1 ; c1ccc2c(-c3ccc(N(c4ccc5sc6ccccc6c5c4)c4ccc5c6ccccc6n(-c6ccc(-c7cccc8ccccc78)cc6)c5c4)cc3)cccc2c1 ; c1ccc2c(-c3ccc(N(c4ccc5c6ccccc6n(-c6ccc(-c7cccc8ccccc78)cc6)c5c4)c4cccc5c4sc4ccccc45)cc3)cccc2c1] 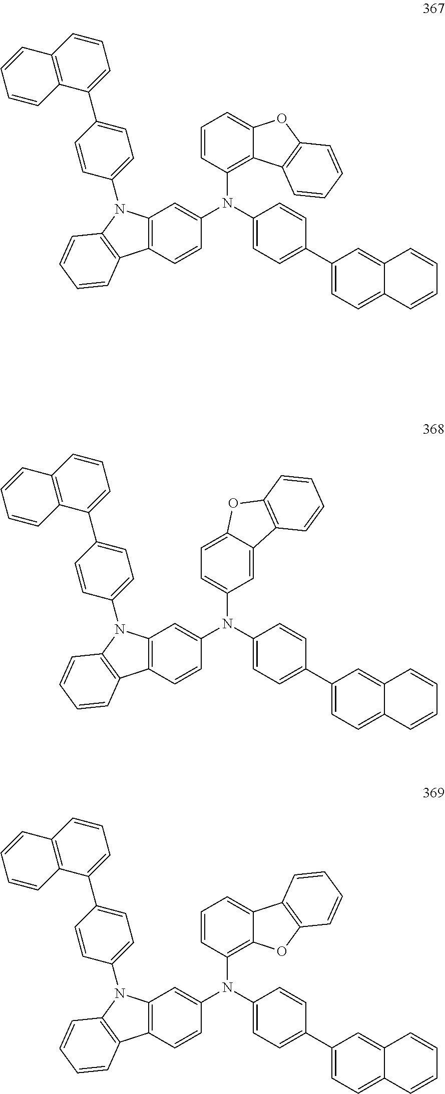 [SMILES: c1ccc2cc(-c3ccc(N(c4ccc5c6ccccc6n(-c6ccc(-c7cccc8ccccc78)cc6)c5c4)c4cccc5oc6ccccc6c45)cc3)ccc2c1 ; c1ccc2cc(-c3ccc(N(c4ccc5oc6ccccc6c5c4)c4ccc5c6ccccc6n(-c6ccc(-c7cccc8ccccc78)cc6)c5c4)cc3)ccc2c1 ; c1ccc2cc(-c3ccc(N(c4ccc5c6ccccc6n(-c6ccc(-c7cccc8ccccc78)cc6)c5c4)c4cccc5c4oc4ccccc45)cc3)ccc2c1] 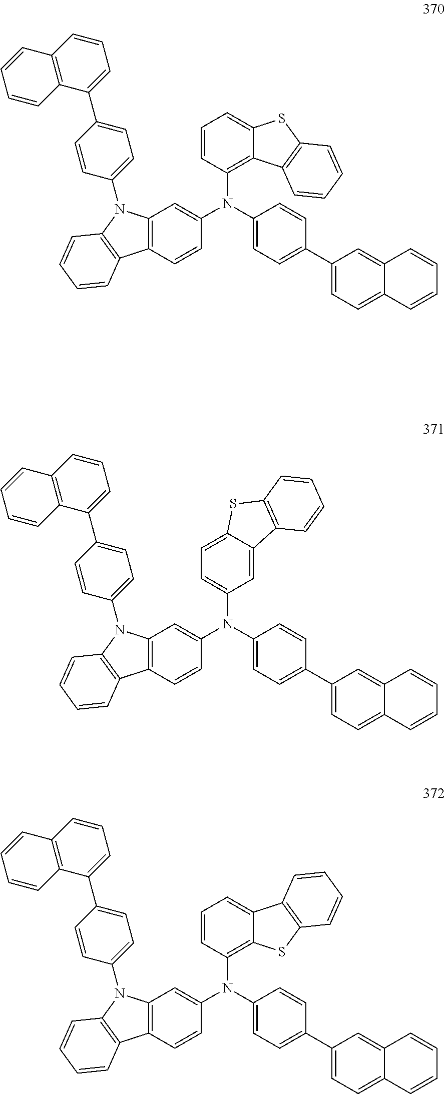 [SMILES: c1ccc2cc(-c3ccc(N(c4ccc5c6ccccc6n(-c6ccc(-c7cccc8ccccc78)cc6)c5c4)c4cccc5sc6ccccc6c45)cc3)ccc2c1 ; c1ccc2cc(-c3ccc(N(c4ccc5sc6ccccc6c5c4)c4ccc5c6ccccc6n(-c6ccc(-c7cccc8ccccc78)cc6)c5c4)cc3)ccc2c1 ; c1ccc2cc(-c3ccc(N(c4ccc5c6ccccc6n(-c6ccc(-c7cccc8ccccc78)cc6)c5c4)c4cccc5c4sc4ccccc45)cc3)ccc2c1] 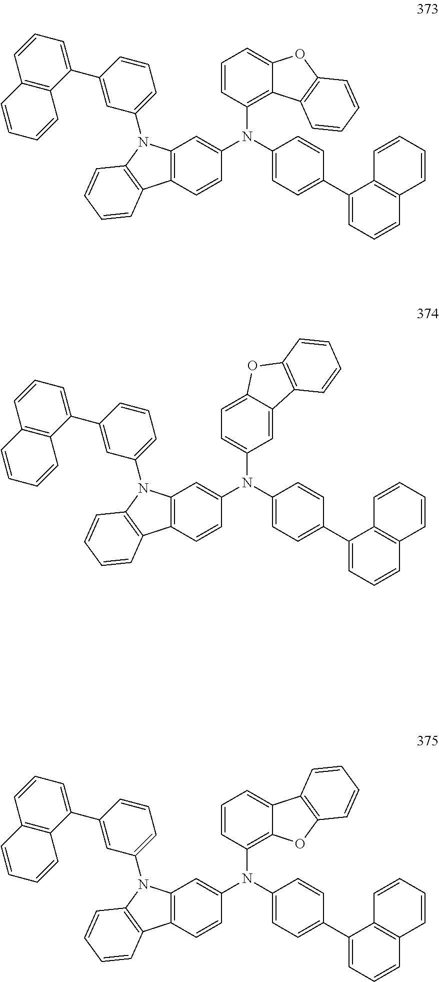 [SMILES: c1cc(-c2cccc3ccccc23)cc(-n2c3ccccc3c3ccc(N(c4ccc(-c5cccc6ccccc56)cc4)c4cccc5oc6ccccc6c45)cc32)c1 ; c1cc(-c2cccc3ccccc23)cc(-n2c3ccccc3c3ccc(N(c4ccc(-c5cccc6ccccc56)cc4)c4ccc5oc6ccccc6c5c4)cc32)c1 ; c1cc(-c2cccc3ccccc23)cc(-n2c3ccccc3c3ccc(N(c4ccc(-c5cccc6ccccc56)cc4)c4cccc5c4oc4ccccc45)cc32)c1] 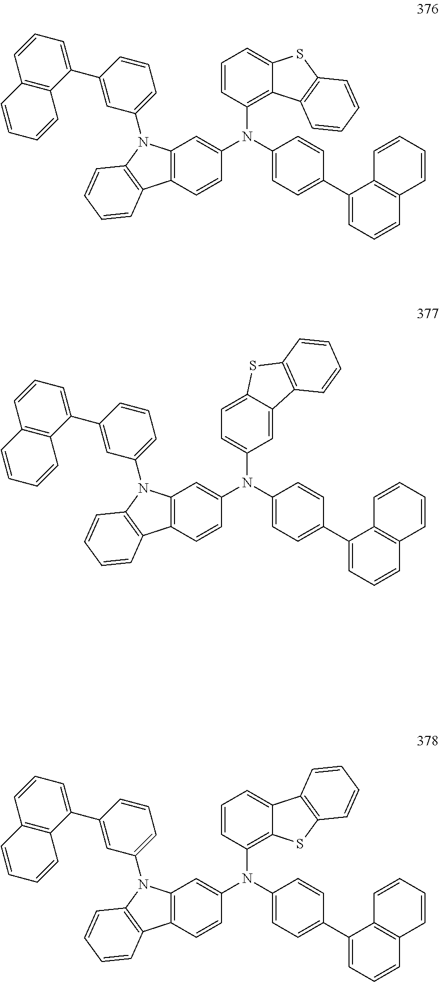 [SMILES: c1cc(-c2cccc3ccccc23)cc(-n2c3ccccc3c3ccc(N(c4ccc(-c5cccc6ccccc56)cc4)c4cccc5sc6ccccc6c45)cc32)c1 ; c1cc(-c2cccc3ccccc23)cc(-n2c3ccccc3c3ccc(N(c4ccc(-c5cccc6ccccc56)cc4)c4ccc5sc6ccccc6c5c4)cc32)c1 ; c1cc(-c2cccc3ccccc23)cc(-n2c3ccccc3c3ccc(N(c4ccc(-c5cccc6ccccc56)cc4)c4cccc5c4sc4ccccc45)cc32)c1] 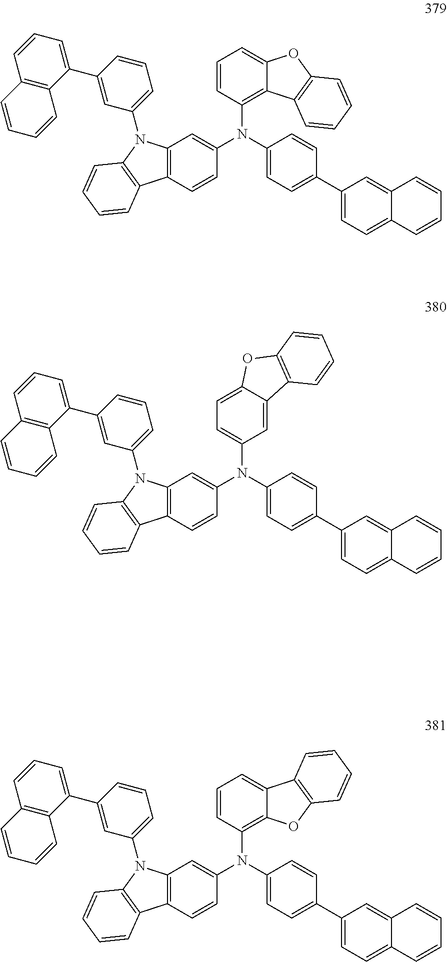 [SMILES: c1cc(-c2cccc3ccccc23)cc(-n2c3ccccc3c3ccc(N(c4ccc(-c5ccc6ccccc6c5)cc4)c4cccc5oc6ccccc6c45)cc32)c1 ; c1cc(-c2cccc3ccccc23)cc(-n2c3ccccc3c3ccc(N(c4ccc(-c5ccc6ccccc6c5)cc4)c4ccc5oc6ccccc6c5c4)cc32)c1 ; c1cc(-c2cccc3ccccc23)cc(-n2c3ccccc3c3ccc(N(c4ccc(-c5ccc6ccccc6c5)cc4)c4cccc5c4oc4ccccc45)cc32)c1] 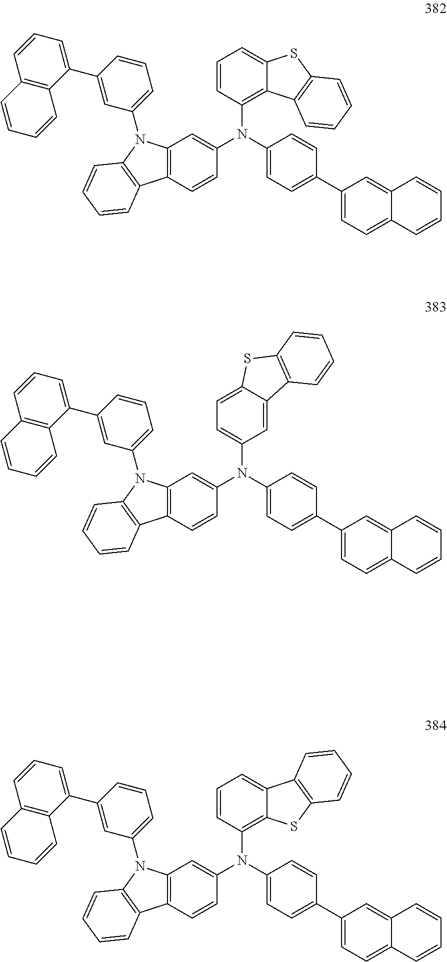 [SMILES: c1cc(-c2cccc3ccccc23)cc(-n2c3ccccc3c3ccc(N(c4ccc(-c5ccc6ccccc6c5)cc4)c4cccc5sc6ccccc6c45)cc32)c1 ; c1cc(-c2cccc3ccccc23)cc(-n2c3ccccc3c3ccc(N(c4ccc(-c5ccc6ccccc6c5)cc4)c4ccc5sc6ccccc6c5c4)cc32)c1 ; c1cc(-c2cccc3ccccc23)cc(-n2c3ccccc3c3ccc(N(c4ccc(-c5ccc6ccccc6c5)cc4)c4cccc5c4sc4ccccc45)cc32)c1] 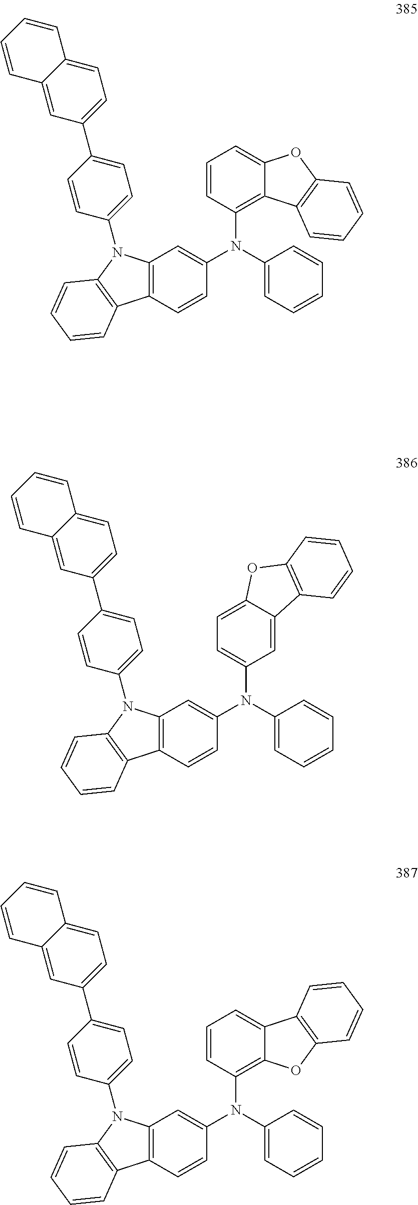 [SMILES: c1ccc(N(c2ccc3c4ccccc4n(-c4ccc(-c5ccc6ccccc6c5)cc4)c3c2)c2cccc3oc4ccccc4c23)cc1 ; c1ccc(N(c2ccc3oc4ccccc4c3c2)c2ccc3c4ccccc4n(-c4ccc(-c5ccc6ccccc6c5)cc4)c3c2)cc1 ; c1ccc(N(c2ccc3c4ccccc4n(-c4ccc(-c5ccc6ccccc6c5)cc4)c3c2)c2cccc3c2oc2ccccc23)cc1] 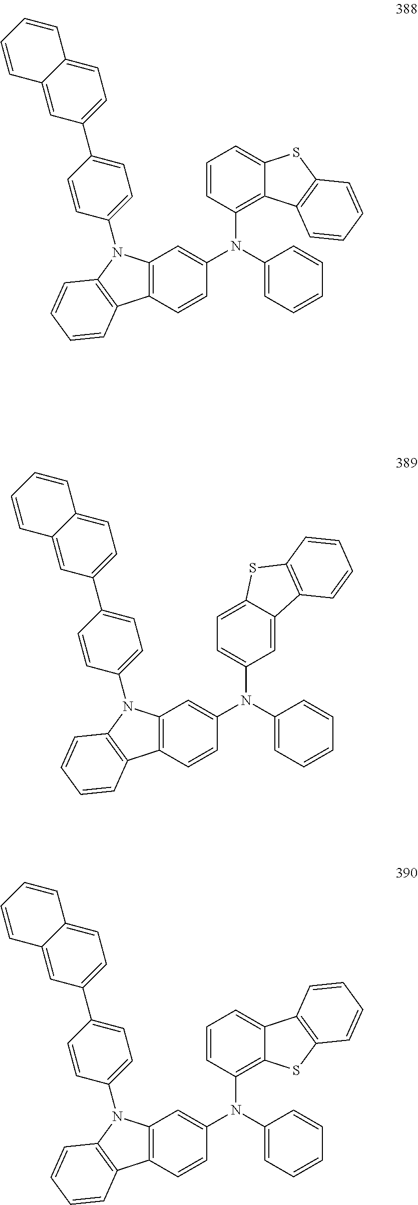 [SMILES: c1ccc(N(c2ccc3c4ccccc4n(-c4ccc(-c5ccc6ccccc6c5)cc4)c3c2)c2cccc3sc4ccccc4c23)cc1 ; c1ccc(N(c2ccc3sc4ccccc4c3c2)c2ccc3c4ccccc4n(-c4ccc(-c5ccc6ccccc6c5)cc4)c3c2)cc1 ; c1ccc(N(c2ccc3c4ccccc4n(-c4ccc(-c5ccc6ccccc6c5)cc4)c3c2)c2cccc3c2sc2ccccc23)cc1] 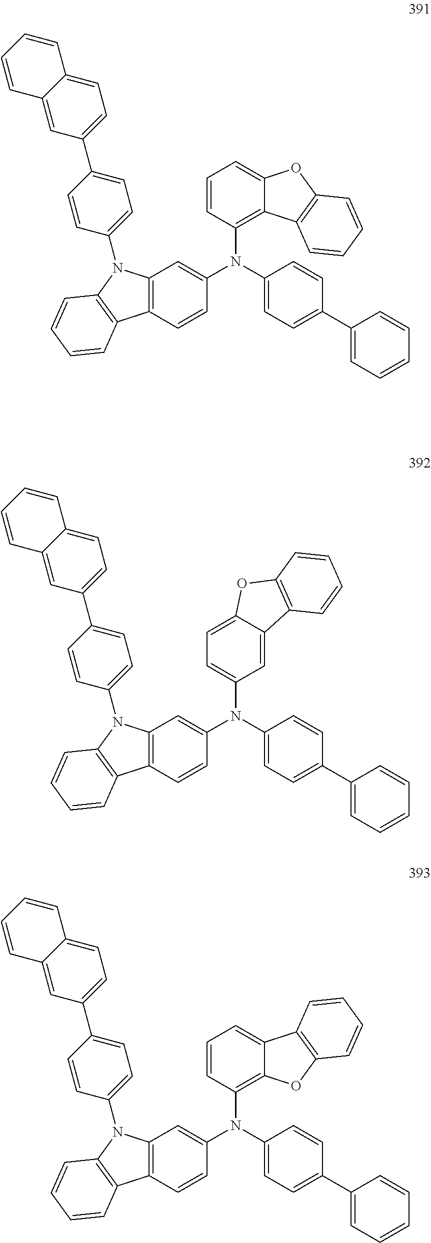 [SMILES: c1ccc(-c2ccc(N(c3ccc4c5ccccc5n(-c5ccc(-c6ccc7ccccc7c6)cc5)c4c3)c3cccc4oc5ccccc5c34)cc2)cc1 ; c1ccc(-c2ccc(N(c3ccc4oc5ccccc5c4c3)c3ccc4c5ccccc5n(-c5ccc(-c6ccc7ccccc7c6)cc5)c4c3)cc2)cc1 ; c1ccc(-c2ccc(N(c3ccc4c5ccccc5n(-c5ccc(-c6ccc7ccccc7c6)cc5)c4c3)c3cccc4c3oc3ccccc34)cc2)cc1] 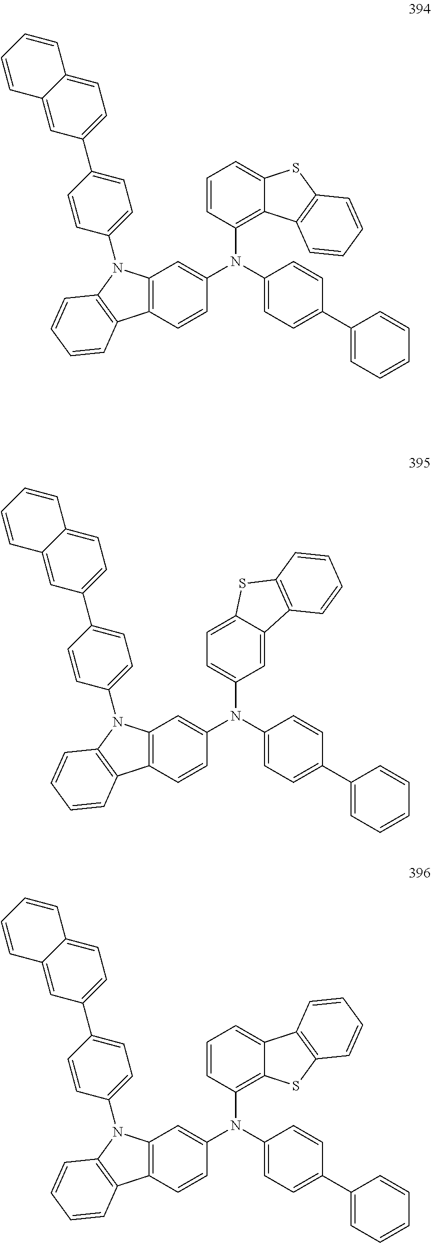 [SMILES: c1ccc(-c2ccc(N(c3ccc4c5ccccc5n(-c5ccc(-c6ccc7ccccc7c6)cc5)c4c3)c3cccc4sc5ccccc5c34)cc2)cc1 ; c1ccc(-c2ccc(N(c3ccc4sc5ccccc5c4c3)c3ccc4c5ccccc5n(-c5ccc(-c6ccc7ccccc7c6)cc5)c4c3)cc2)cc1 ; c1ccc(-c2ccc(N(c3ccc4c5ccccc5n(-c5ccc(-c6ccc7ccccc7c6)cc5)c4c3)c3cccc4c3sc3ccccc34)cc2)cc1] 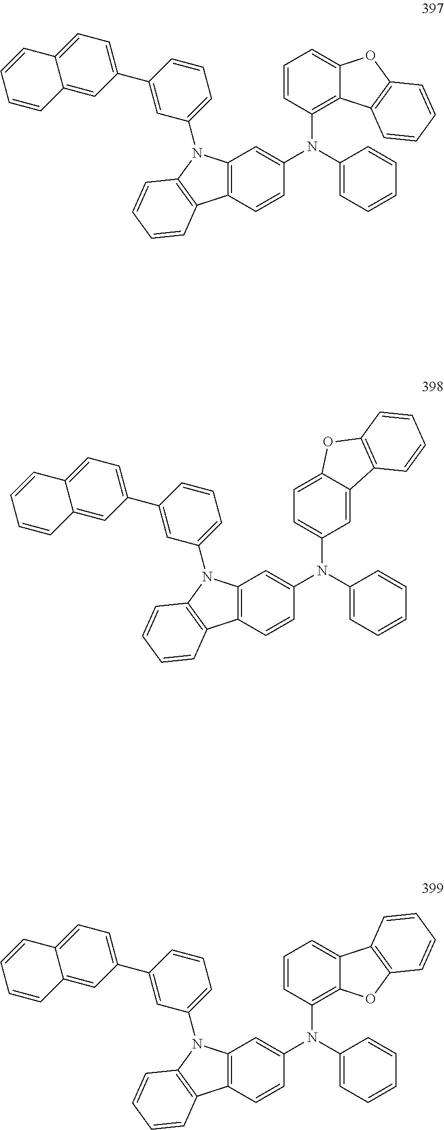 [SMILES: c1ccc(N(c2ccc3c4ccccc4n(-c4cccc(-c5ccc6ccccc6c5)c4)c3c2)c2cccc3oc4ccccc4c23)cc1 ; c1ccc(N(c2ccc3oc4ccccc4c3c2)c2ccc3c4ccccc4n(-c4cccc(-c5ccc6ccccc6c5)c4)c3c2)cc1 ; c1ccc(N(c2ccc3c4ccccc4n(-c4cccc(-c5ccc6ccccc6c5)c4)c3c2)c2cccc3c2oc2ccccc23)cc1] 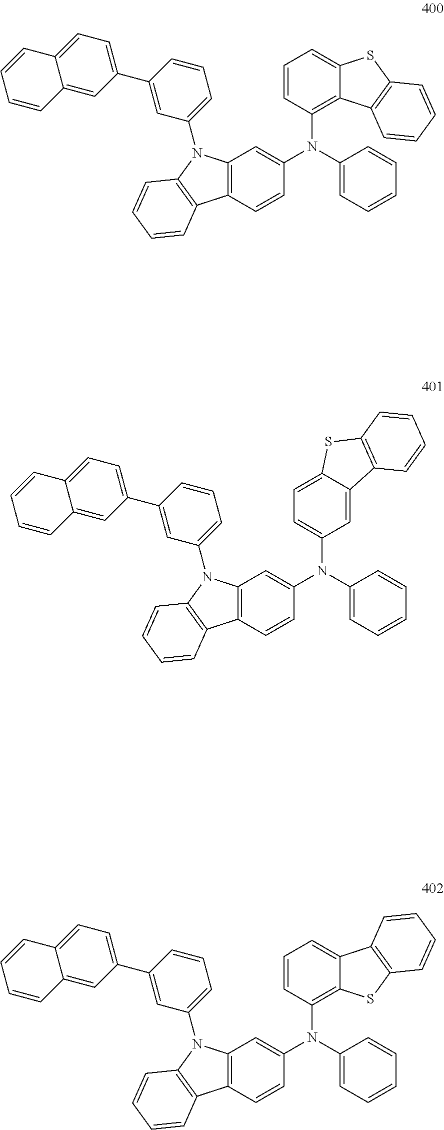 [SMILES: c1ccc(N(c2ccc3c4ccccc4n(-c4cccc(-c5ccc6ccccc6c5)c4)c3c2)c2cccc3sc4ccccc4c23)cc1 ; c1ccc(N(c2ccc3sc4ccccc4c3c2)c2ccc3c4ccccc4n(-c4cccc(-c5ccc6ccccc6c5)c4)c3c2)cc1 ; c1ccc(N(c2ccc3c4ccccc4n(-c4cccc(-c5ccc6ccccc6c5)c4)c3c2)c2cccc3c2sc2ccccc23)cc1] 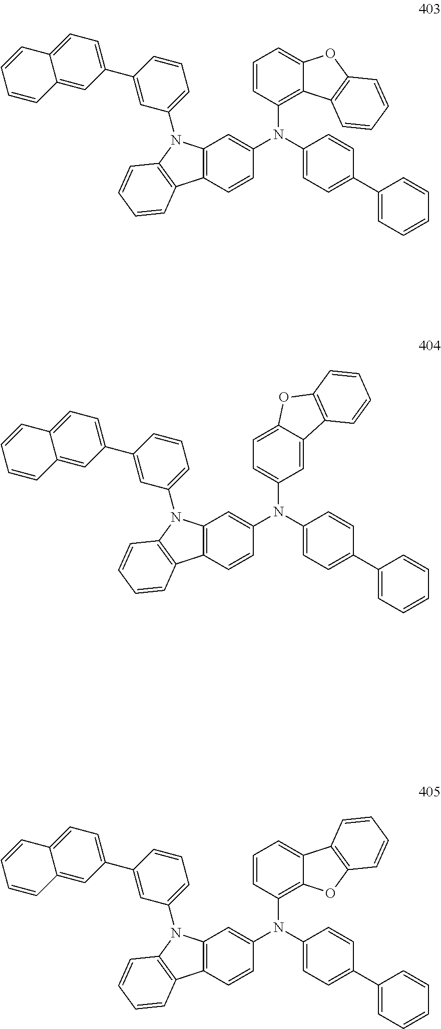 [SMILES: c1ccc(-c2ccc(N(c3ccc4c5ccccc5n(-c5cccc(-c6ccc7ccccc7c6)c5)c4c3)c3cccc4oc5ccccc5c34)cc2)cc1 ; c1ccc(-c2ccc(N(c3ccc4oc5ccccc5c4c3)c3ccc4c5ccccc5n(-c5cccc(-c6ccc7ccccc7c6)c5)c4c3)cc2)cc1 ; c1ccc(-c2ccc(N(c3ccc4c5ccccc5n(-c5cccc(-c6ccc7ccccc7c6)c5)c4c3)c3cccc4c3oc3ccccc34)cc2)cc1] 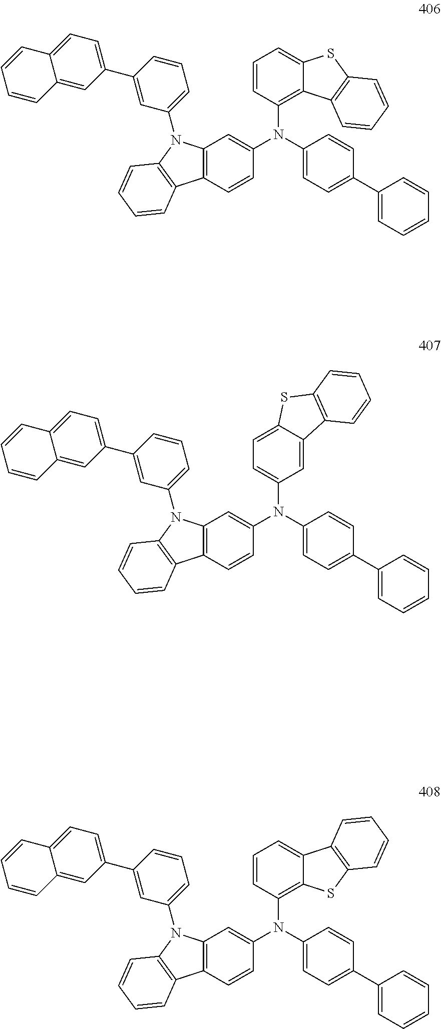 [SMILES: c1ccc(-c2ccc(N(c3ccc4c5ccccc5n(-c5cccc(-c6ccc7ccccc7c6)c5)c4c3)c3cccc4sc5ccccc5c34)cc2)cc1 ; c1ccc(-c2ccc(N(c3ccc4sc5ccccc5c4c3)c3ccc4c5ccccc5n(-c5cccc(-c6ccc7ccccc7c6)c5)c4c3)cc2)cc1 ; c1ccc(-c2ccc(N(c3ccc4c5ccccc5n(-c5cccc(-c6ccc7ccccc7c6)c5)c4c3)c3cccc4c3sc3ccccc34)cc2)cc1] 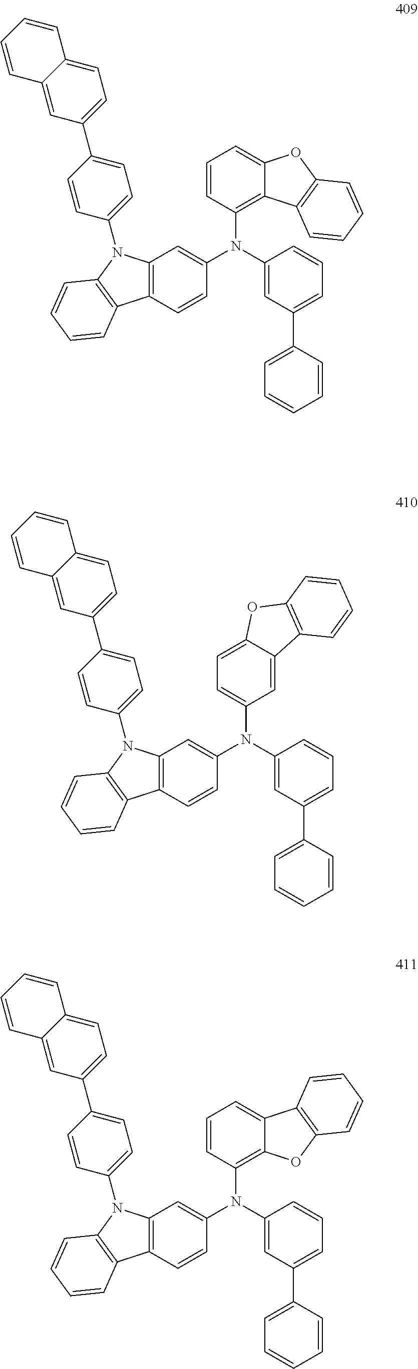 [SMILES: c1ccc(-c2cccc(N(c3ccc4c5ccccc5n(-c5ccc(-c6ccc7ccccc7c6)cc5)c4c3)c3cccc4oc5ccccc5c34)c2)cc1 ; c1ccc(-c2cccc(N(c3ccc4oc5ccccc5c4c3)c3ccc4c5ccccc5n(-c5ccc(-c6ccc7ccccc7c6)cc5)c4c3)c2)cc1 ; c1ccc(-c2cccc(N(c3ccc4c5ccccc5n(-c5ccc(-c6ccc7ccccc7c6)cc5)c4c3)c3cccc4c3oc3ccccc34)c2)cc1] 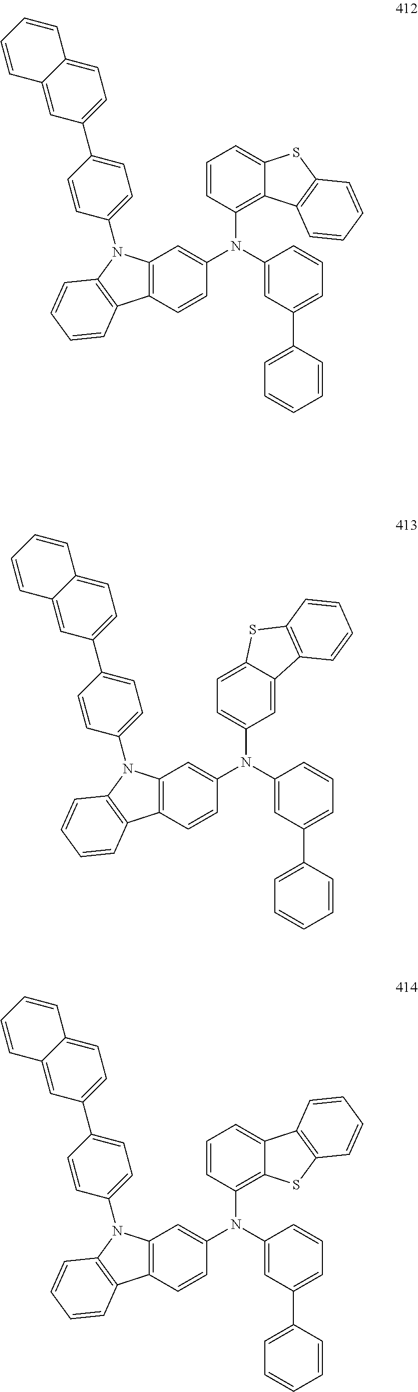 [SMILES: c1ccc(-c2cccc(N(c3ccc4c5ccccc5n(-c5ccc(-c6ccc7ccccc7c6)cc5)c4c3)c3cccc4sc5ccccc5c34)c2)cc1 ; c1ccc(-c2cccc(N(c3ccc4sc5ccccc5c4c3)c3ccc4c5ccccc5n(-c5ccc(-c6ccc7ccccc7c6)cc5)c4c3)c2)cc1 ; c1ccc(-c2cccc(N(c3ccc4c5ccccc5n(-c5ccc(-c6ccc7ccccc7c6)cc5)c4c3)c3cccc4c3sc3ccccc34)c2)cc1] 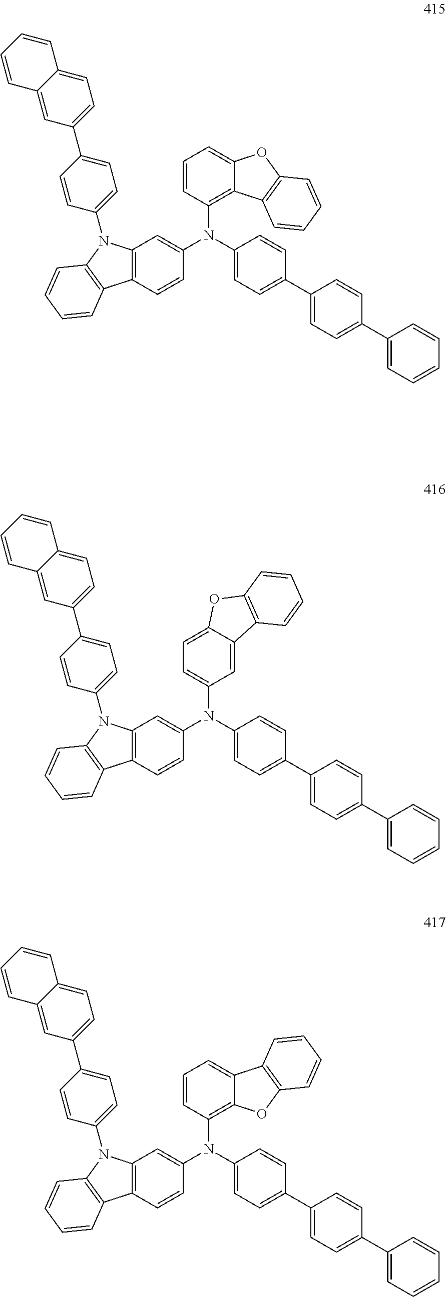 [SMILES: c1ccc(-c2ccc(-c3ccc(N(c4ccc5c6ccccc6n(-c6ccc(-c7ccc8ccccc8c7)cc6)c5c4)c4cccc5oc6ccccc6c45)cc3)cc2)cc1 ; c1ccc(-c2ccc(-c3ccc(N(c4ccc5oc6ccccc6c5c4)c4ccc5c6ccccc6n(-c6ccc(-c7ccc8ccccc8c7)cc6)c5c4)cc3)cc2)cc1 ; c1ccc(-c2ccc(-c3ccc(N(c4ccc5c6ccccc6n(-c6ccc(-c7ccc8ccccc8c7)cc6)c5c4)c4cccc5c4oc4ccccc45)cc3)cc2)cc1] 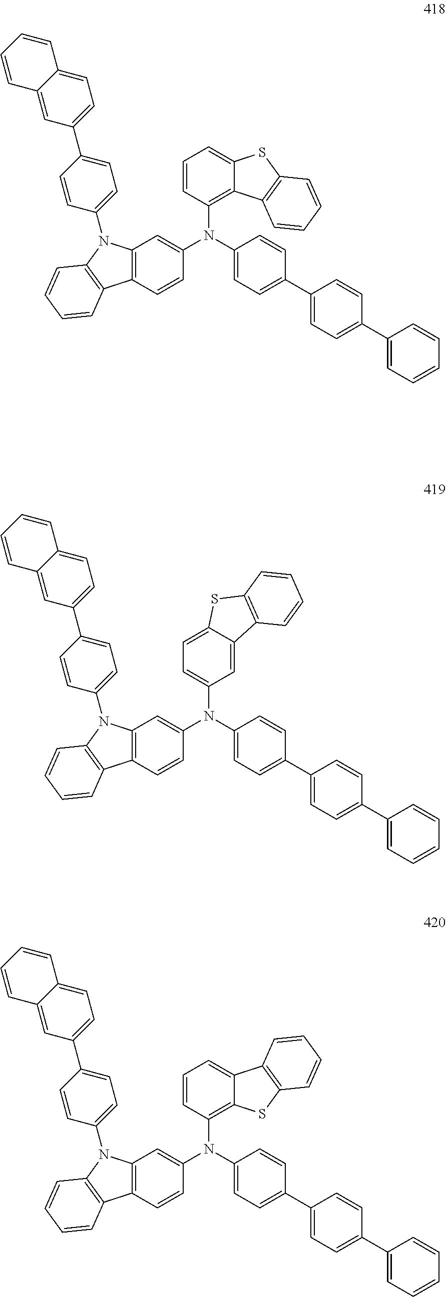 [SMILES: c1ccc(-c2ccc(-c3ccc(N(c4ccc5c6ccccc6n(-c6ccc(-c7ccc8ccccc8c7)cc6)c5c4)c4cccc5sc6ccccc6c45)cc3)cc2)cc1 ; c1ccc(-c2ccc(-c3ccc(N(c4ccc5sc6ccccc6c5c4)c4ccc5c6ccccc6n(-c6ccc(-c7ccc8ccccc8c7)cc6)c5c4)cc3)cc2)cc1 ; c1ccc(-c2ccc(-c3ccc(N(c4ccc5c6ccccc6n(-c6ccc(-c7ccc8ccccc8c7)cc6)c5c4)c4cccc5c4sc4ccccc45)cc3)cc2)cc1] 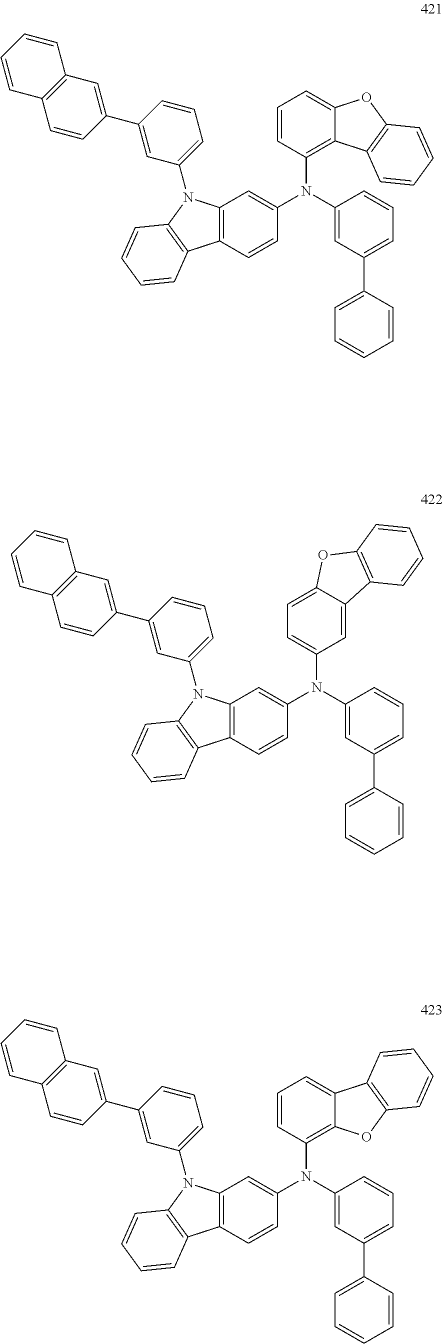 [SMILES: c1ccc(-c2cccc(N(c3ccc4c5ccccc5n(-c5cccc(-c6ccc7ccccc7c6)c5)c4c3)c3cccc4oc5ccccc5c34)c2)cc1 ; c1ccc(-c2cccc(N(c3ccc4oc5ccccc5c4c3)c3ccc4c5ccccc5n(-c5cccc(-c6ccc7ccccc7c6)c5)c4c3)c2)cc1 ; c1ccc(-c2cccc(N(c3ccc4c5ccccc5n(-c5cccc(-c6ccc7ccccc7c6)c5)c4c3)c3cccc4c3oc3ccccc34)c2)cc1] 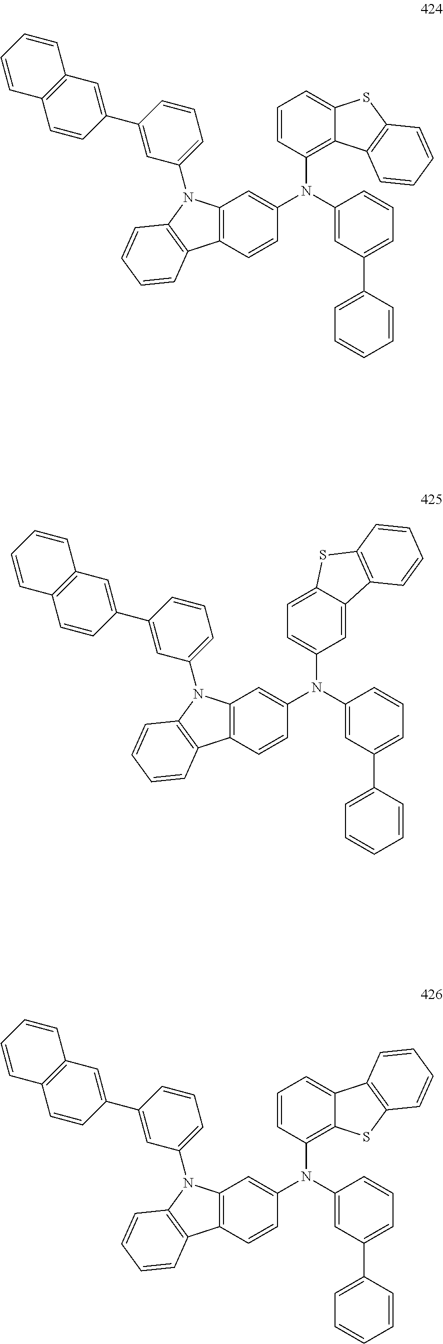 [SMILES: c1ccc(-c2cccc(N(c3ccc4c5ccccc5n(-c5cccc(-c6ccc7ccccc7c6)c5)c4c3)c3cccc4sc5ccccc5c34)c2)cc1 ; c1ccc(-c2cccc(N(c3ccc4sc5ccccc5c4c3)c3ccc4c5ccccc5n(-c5cccc(-c6ccc7ccccc7c6)c5)c4c3)c2)cc1 ; c1ccc(-c2cccc(N(c3ccc4c5ccccc5n(-c5cccc(-c6ccc7ccccc7c6)c5)c4c3)c3cccc4c3sc3ccccc34)c2)cc1] 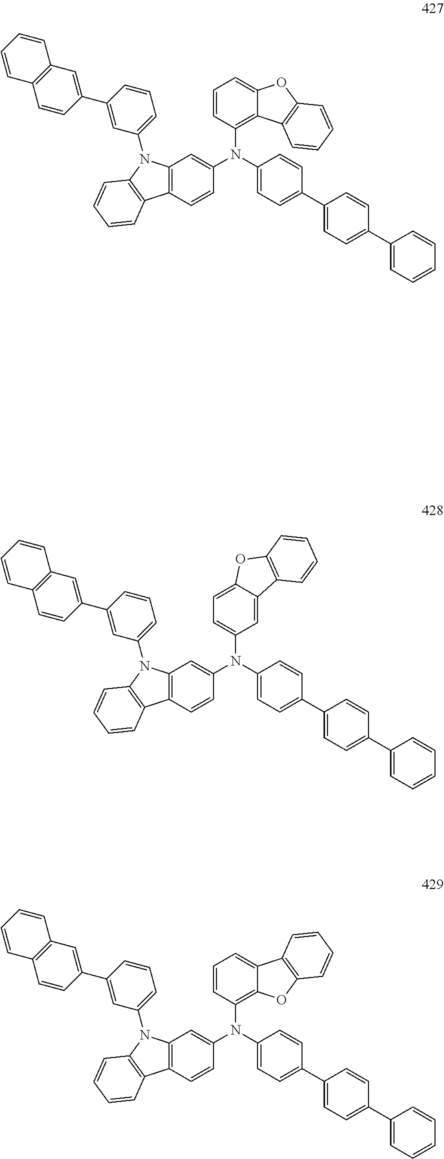 [SMILES: c1ccc(-c2ccc(-c3ccc(N(c4ccc5c6ccccc6n(-c6cccc(-c7ccc8ccccc8c7)c6)c5c4)c4cccc5oc6ccccc6c45)cc3)cc2)cc1 ; c1ccc(-c2ccc(-c3ccc(N(c4ccc5oc6ccccc6c5c4)c4ccc5c6ccccc6n(-c6cccc(-c7ccc8ccccc8c7)c6)c5c4)cc3)cc2)cc1 ; c1ccc(-c2ccc(-c3ccc(N(c4ccc5c6ccccc6n(-c6cccc(-c7ccc8ccccc8c7)c6)c5c4)c4cccc5c4oc4ccccc45)cc3)cc2)cc1] 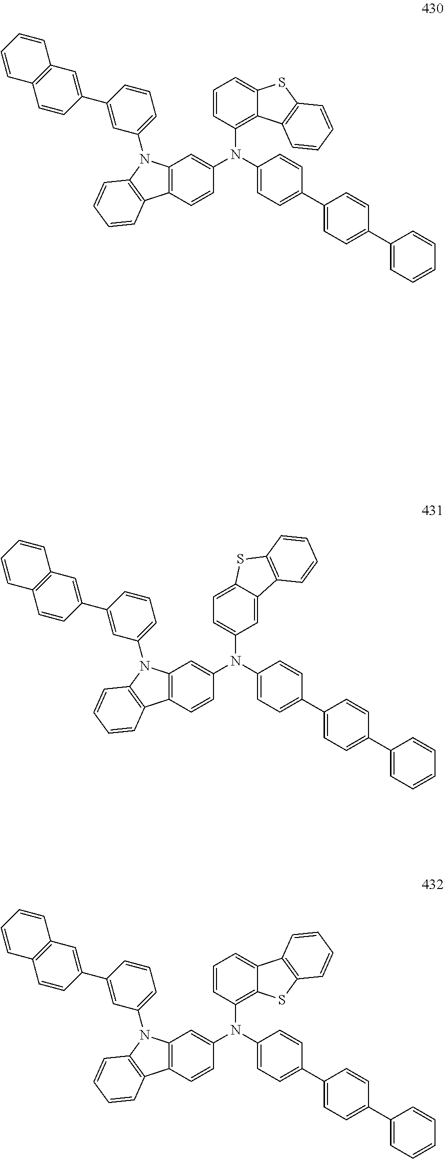 [SMILES: c1ccc(-c2ccc(-c3ccc(N(c4ccc5c6ccccc6n(-c6cccc(-c7ccc8ccccc8c7)c6)c5c4)c4cccc5sc6ccccc6c45)cc3)cc2)cc1 ; c1ccc(-c2ccc(-c3ccc(N(c4ccc5sc6ccccc6c5c4)c4ccc5c6ccccc6n(-c6cccc(-c7ccc8ccccc8c7)c6)c5c4)cc3)cc2)cc1 ; c1ccc(-c2ccc(-c3ccc(N(c4ccc5c6ccccc6n(-c6cccc(-c7ccc8ccccc8c7)c6)c5c4)c4cccc5c4sc4ccccc45)cc3)cc2)cc1] 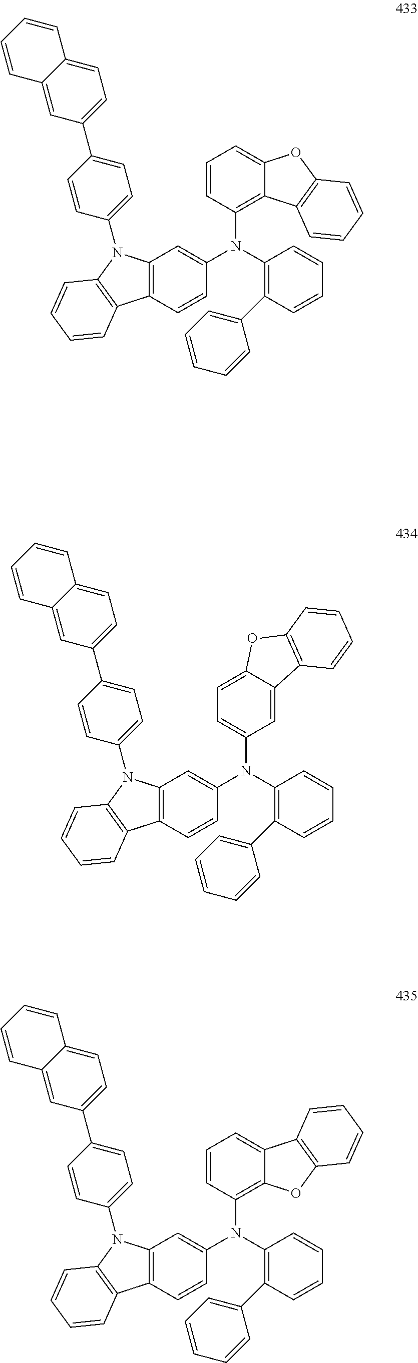 [SMILES: c1ccc(-c2ccccc2N(c2ccc3c4ccccc4n(-c4ccc(-c5ccc6ccccc6c5)cc4)c3c2)c2cccc3oc4ccccc4c23)cc1 ; c1ccc(-c2ccccc2N(c2ccc3oc4ccccc4c3c2)c2ccc3c4ccccc4n(-c4ccc(-c5ccc6ccccc6c5)cc4)c3c2)cc1 ; c1ccc(-c2ccccc2N(c2ccc3c4ccccc4n(-c4ccc(-c5ccc6ccccc6c5)cc4)c3c2)c2cccc3c2oc2ccccc23)cc1] 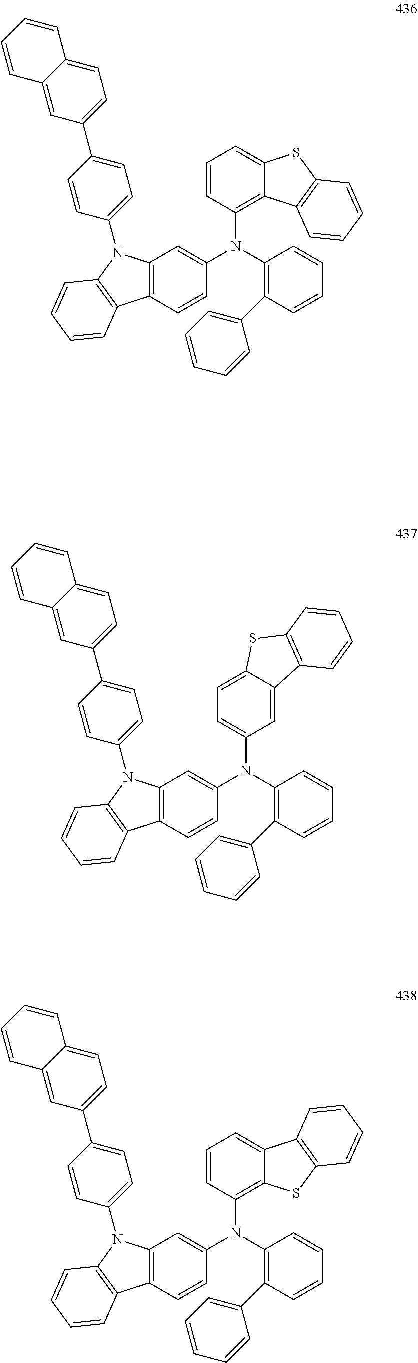 [SMILES: c1ccc(-c2ccccc2N(c2ccc3c4ccccc4n(-c4ccc(-c5ccc6ccccc6c5)cc4)c3c2)c2cccc3sc4ccccc4c23)cc1 ; c1ccc(-c2ccccc2N(c2ccc3sc4ccccc4c3c2)c2ccc3c4ccccc4n(-c4ccc(-c5ccc6ccccc6c5)cc4)c3c2)cc1 ; c1ccc(-c2ccccc2N(c2ccc3c4ccccc4n(-c4ccc(-c5ccc6ccccc6c5)cc4)c3c2)c2cccc3c2sc2ccccc23)cc1] 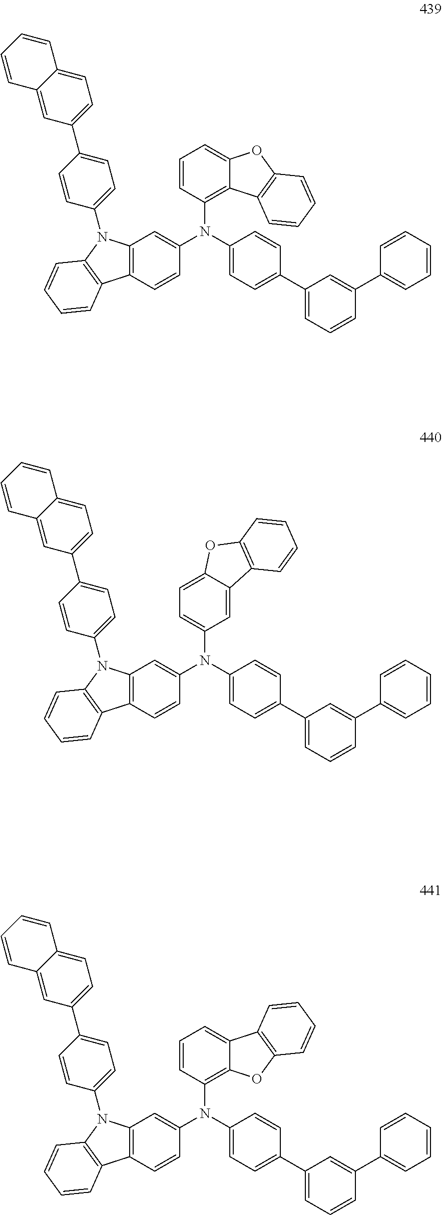 [SMILES: c1ccc(-c2cccc(-c3ccc(N(c4ccc5c6ccccc6n(-c6ccc(-c7ccc8ccccc8c7)cc6)c5c4)c4cccc5oc6ccccc6c45)cc3)c2)cc1 ; c1ccc(-c2cccc(-c3ccc(N(c4ccc5oc6ccccc6c5c4)c4ccc5c6ccccc6n(-c6ccc(-c7ccc8ccccc8c7)cc6)c5c4)cc3)c2)cc1 ; c1ccc(-c2cccc(-c3ccc(N(c4ccc5c6ccccc6n(-c6ccc(-c7ccc8ccccc8c7)cc6)c5c4)c4cccc5c4oc4ccccc45)cc3)c2)cc1] 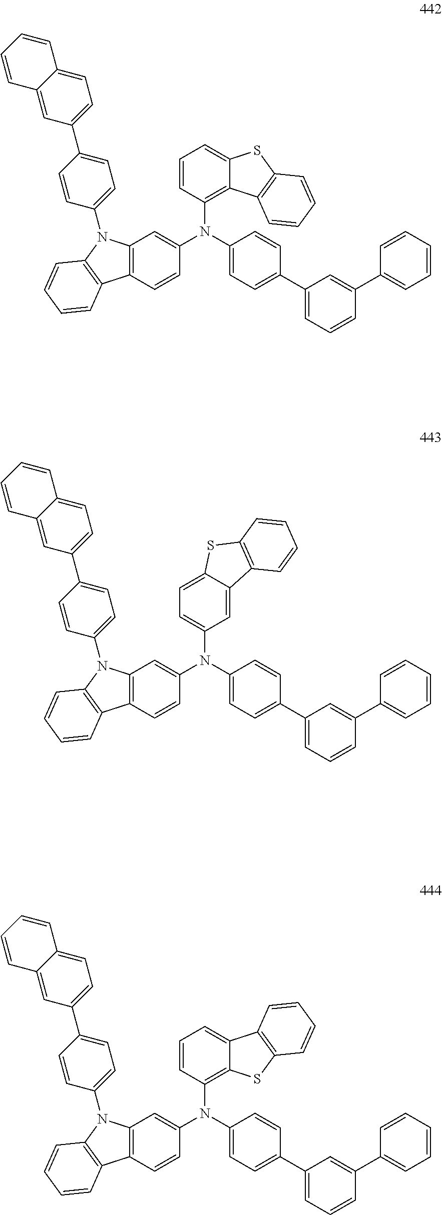 [SMILES: c1ccc(-c2cccc(-c3ccc(N(c4ccc5c6ccccc6n(-c6ccc(-c7ccc8ccccc8c7)cc6)c5c4)c4cccc5sc6ccccc6c45)cc3)c2)cc1 ; c1ccc(-c2cccc(-c3ccc(N(c4ccc5sc6ccccc6c5c4)c4ccc5c6ccccc6n(-c6ccc(-c7ccc8ccccc8c7)cc6)c5c4)cc3)c2)cc1 ; c1ccc(-c2cccc(-c3ccc(N(c4ccc5c6ccccc6n(-c6ccc(-c7ccc8ccccc8c7)cc6)c5c4)c4cccc5c4sc4ccccc45)cc3)c2)cc1] 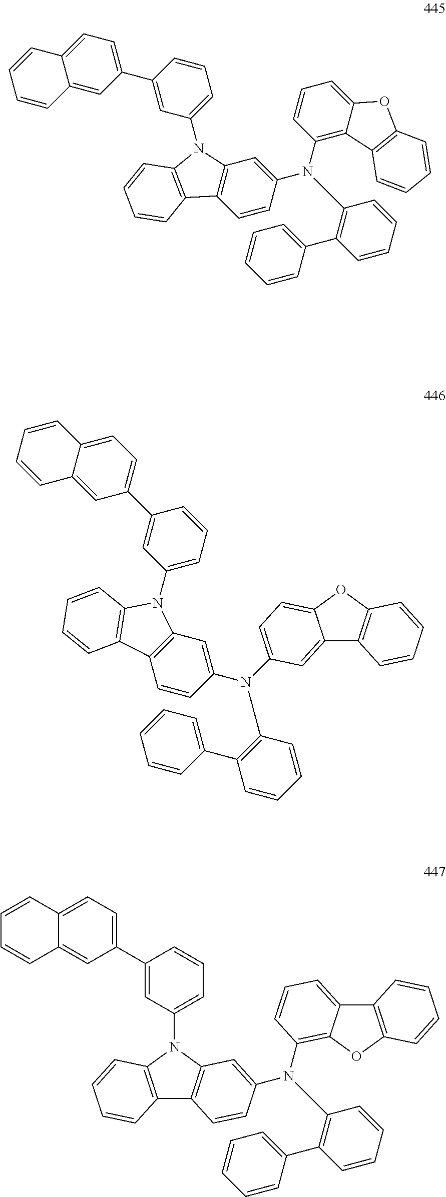 [SMILES: c1ccc(-c2ccccc2N(c2ccc3c4ccccc4n(-c4cccc(-c5ccc6ccccc6c5)c4)c3c2)c2cccc3oc4ccccc4c23)cc1 ; c1ccc(-c2ccccc2N(c2ccc3oc4ccccc4c3c2)c2ccc3c4ccccc4n(-c4cccc(-c5ccc6ccccc6c5)c4)c3c2)cc1 ; c1ccc(-c2ccccc2N(c2ccc3c4ccccc4n(-c4cccc(-c5ccc6ccccc6c5)c4)c3c2)c2cccc3c2oc2ccccc23)cc1] 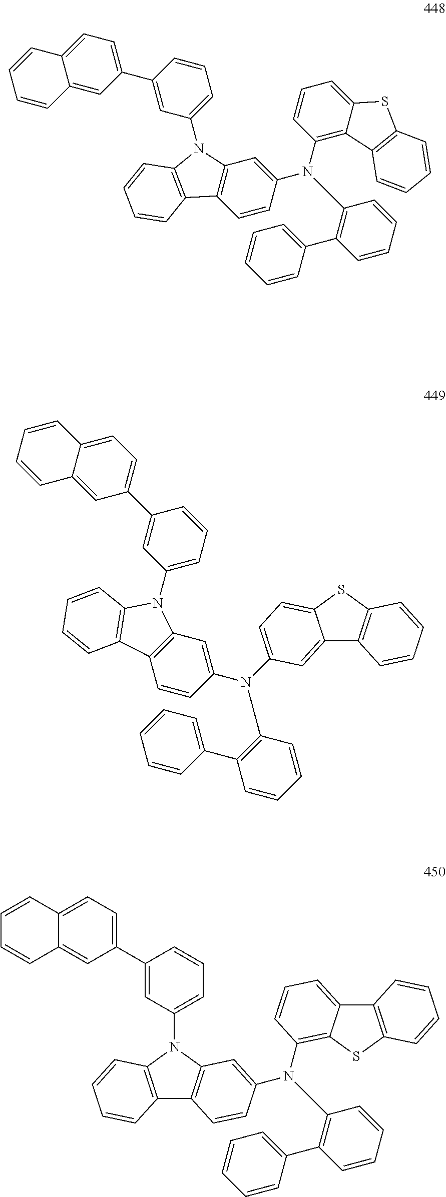 [SMILES: c1ccc(-c2ccccc2N(c2ccc3c4ccccc4n(-c4cccc(-c5ccc6ccccc6c5)c4)c3c2)c2cccc3sc4ccccc4c23)cc1 ; c1ccc(-c2ccccc2N(c2ccc3sc4ccccc4c3c2)c2ccc3c4ccccc4n(-c4cccc(-c5ccc6ccccc6c5)c4)c3c2)cc1 ; c1ccc(-c2ccccc2N(c2ccc3c4ccccc4n(-c4cccc(-c5ccc6ccccc6c5)c4)c3c2)c2cccc3c2sc2ccccc23)cc1] 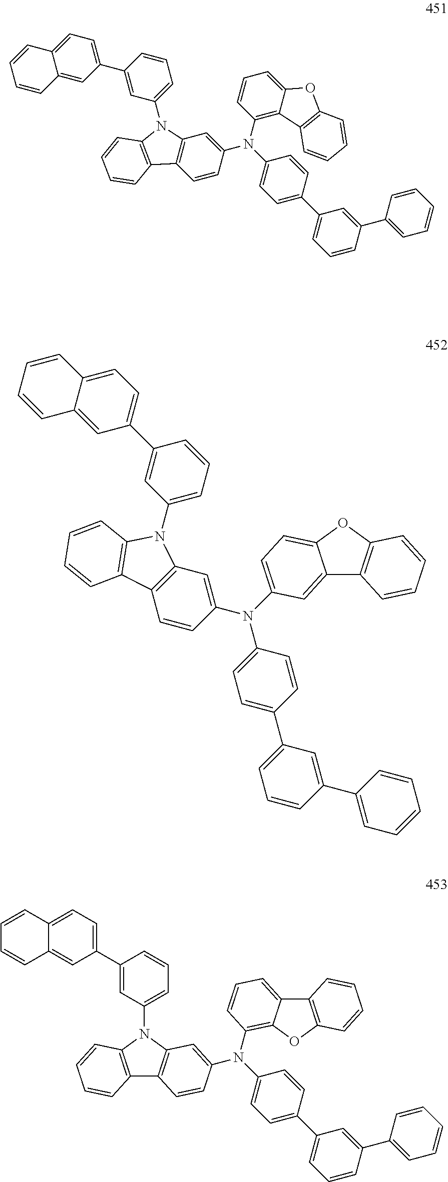 [SMILES: c1ccc(-c2cccc(-c3ccc(N(c4ccc5c6ccccc6n(-c6cccc(-c7ccc8ccccc8c7)c6)c5c4)c4cccc5oc6ccccc6c45)cc3)c2)cc1 ; c1ccc(-c2cccc(-c3ccc(N(c4ccc5oc6ccccc6c5c4)c4ccc5c6ccccc6n(-c6cccc(-c7ccc8ccccc8c7)c6)c5c4)cc3)c2)cc1 ; c1ccc(-c2cccc(-c3ccc(N(c4ccc5c6ccccc6n(-c6cccc(-c7ccc8ccccc8c7)c6)c5c4)c4cccc5c4oc4ccccc45)cc3)c2)cc1] 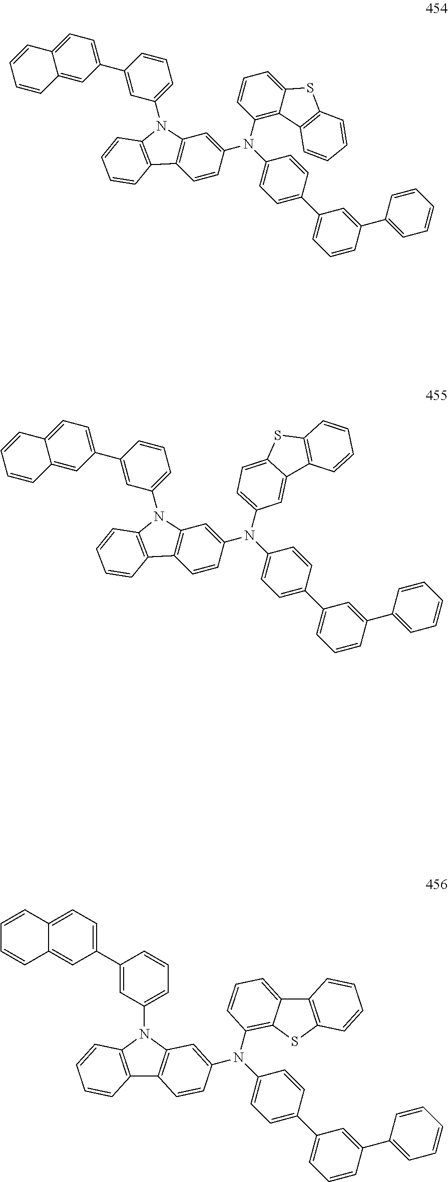 [SMILES: c1ccc(-c2cccc(-c3ccc(N(c4ccc5c6ccccc6n(-c6cccc(-c7ccc8ccccc8c7)c6)c5c4)c4cccc5sc6ccccc6c45)cc3)c2)cc1 ; c1ccc(-c2cccc(-c3ccc(N(c4ccc5sc6ccccc6c5c4)c4ccc5c6ccccc6n(-c6cccc(-c7ccc8ccccc8c7)c6)c5c4)cc3)c2)cc1 ; c1ccc(-c2cccc(-c3ccc(N(c4ccc5c6ccccc6n(-c6cccc(-c7ccc8ccccc8c7)c6)c5c4)c4cccc5c4sc4ccccc45)cc3)c2)cc1] 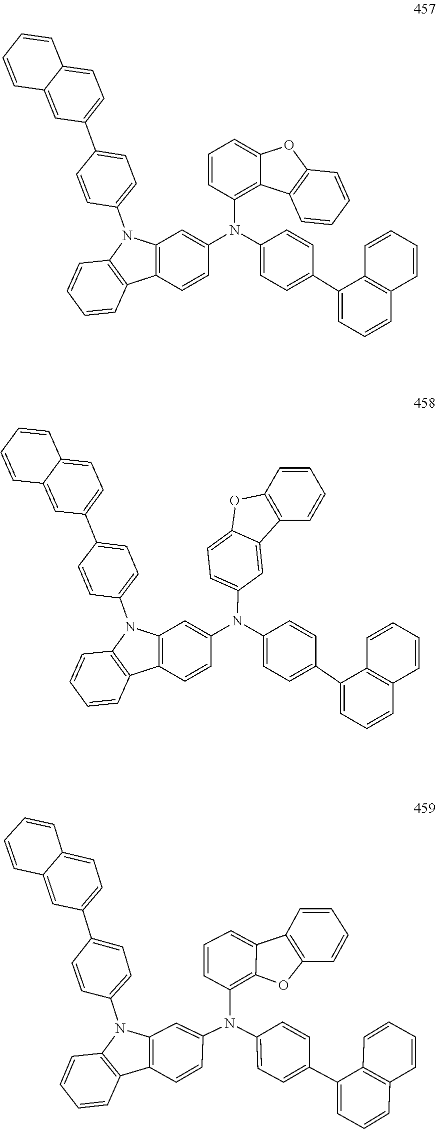 [SMILES: c1ccc2cc(-c3ccc(-n4c5ccccc5c5ccc(N(c6ccc(-c7cccc8ccccc78)cc6)c6cccc7oc8ccccc8c67)cc54)cc3)ccc2c1 ; c1ccc2cc(-c3ccc(-n4c5ccccc5c5ccc(N(c6ccc(-c7cccc8ccccc78)cc6)c6ccc7oc8ccccc8c7c6)cc54)cc3)ccc2c1 ; c1ccc2cc(-c3ccc(-n4c5ccccc5c5ccc(N(c6ccc(-c7cccc8ccccc78)cc6)c6cccc7c6oc6ccccc67)cc54)cc3)ccc2c1] 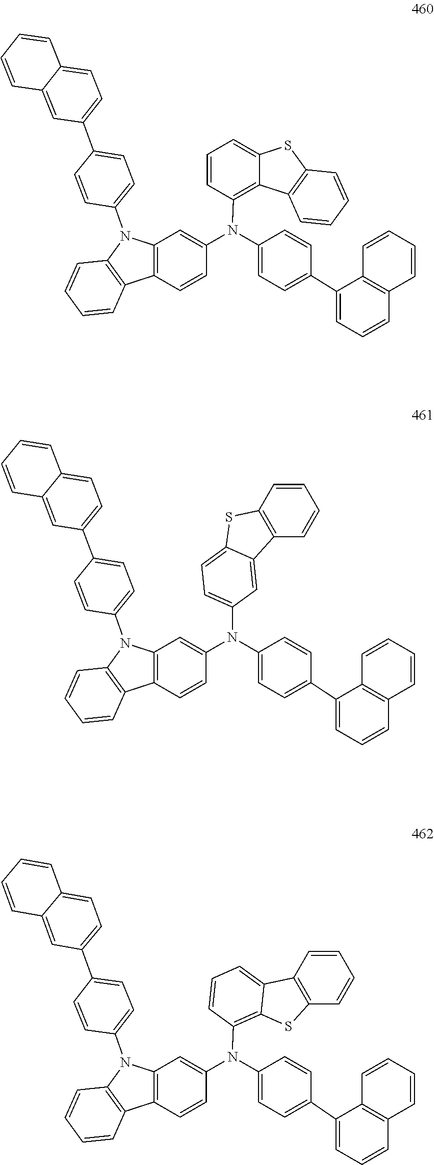 [SMILES: c1ccc2cc(-c3ccc(-n4c5ccccc5c5ccc(N(c6ccc(-c7cccc8ccccc78)cc6)c6cccc7sc8ccccc8c67)cc54)cc3)ccc2c1 ; c1ccc2cc(-c3ccc(-n4c5ccccc5c5ccc(N(c6ccc(-c7cccc8ccccc78)cc6)c6ccc7sc8ccccc8c7c6)cc54)cc3)ccc2c1 ; c1ccc2cc(-c3ccc(-n4c5ccccc5c5ccc(N(c6ccc(-c7cccc8ccccc78)cc6)c6cccc7c6sc6ccccc67)cc54)cc3)ccc2c1] 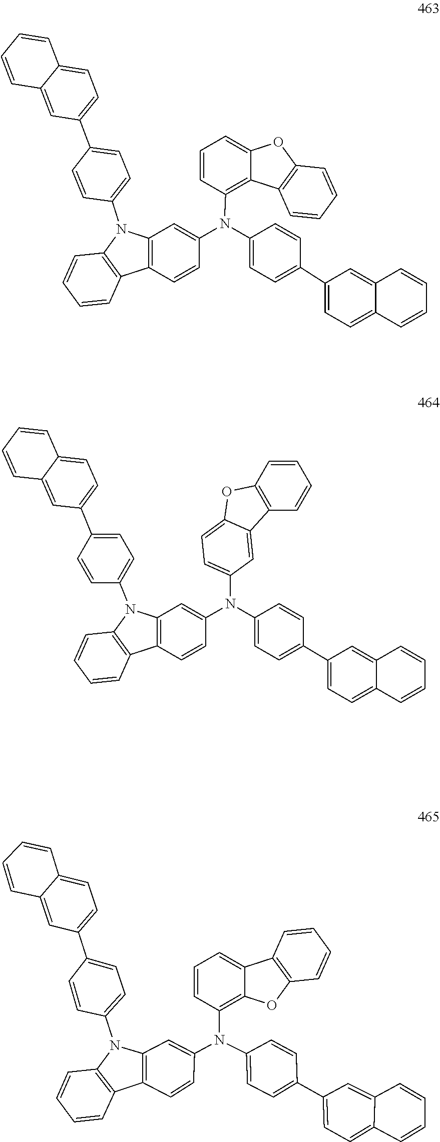 [SMILES: c1ccc2cc(-c3ccc(N(c4ccc5c6ccccc6n(-c6ccc(-c7ccc8ccccc8c7)cc6)c5c4)c4cccc5oc6ccccc6c45)cc3)ccc2c1 ; c1ccc2cc(-c3ccc(N(c4ccc5oc6ccccc6c5c4)c4ccc5c6ccccc6n(-c6ccc(-c7ccc8ccccc8c7)cc6)c5c4)cc3)ccc2c1 ; c1ccc2cc(-c3ccc(N(c4ccc5c6ccccc6n(-c6ccc(-c7ccc8ccccc8c7)cc6)c5c4)c4cccc5c4oc4ccccc45)cc3)ccc2c1] 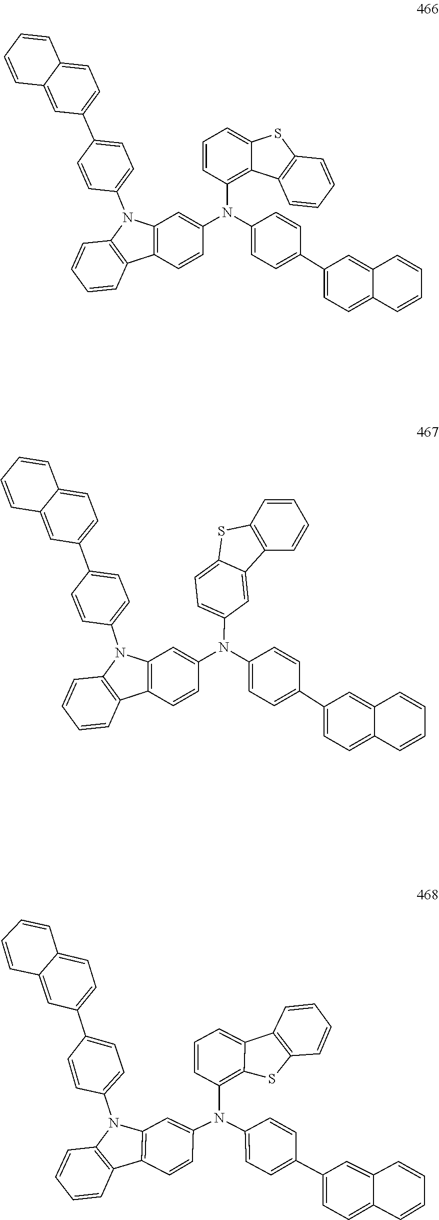 [SMILES: c1ccc2cc(-c3ccc(N(c4ccc5c6ccccc6n(-c6ccc(-c7ccc8ccccc8c7)cc6)c5c4)c4cccc5sc6ccccc6c45)cc3)ccc2c1 ; c1ccc2cc(-c3ccc(N(c4ccc5sc6ccccc6c5c4)c4ccc5c6ccccc6n(-c6ccc(-c7ccc8ccccc8c7)cc6)c5c4)cc3)ccc2c1 ; c1ccc2cc(-c3ccc(N(c4ccc5c6ccccc6n(-c6ccc(-c7ccc8ccccc8c7)cc6)c5c4)c4cccc5c4sc4ccccc45)cc3)ccc2c1] 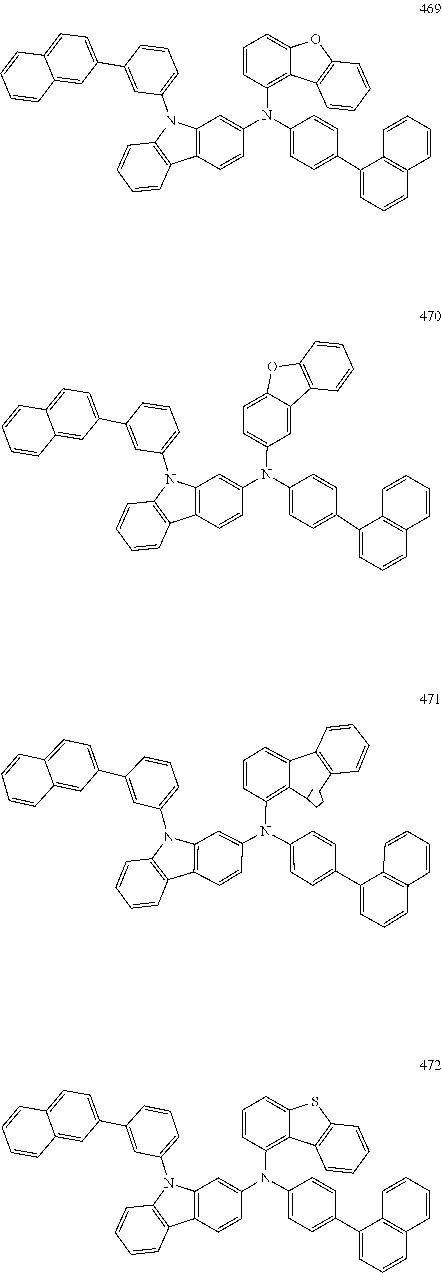 [SMILES: c1cc(-c2ccc3ccccc3c2)cc(-n2c3ccccc3c3ccc(N(c4ccc(-c5cccc6ccccc56)cc4)c4cccc5oc6ccccc6c45)cc32)c1 ; c1cc(-c2ccc3ccccc3c2)cc(-n2c3ccccc3c3ccc(N(c4ccc(-c5cccc6ccccc56)cc4)c4ccc5oc6ccccc6c5c4)cc32)c1 ; CC1CCCc2ccccc2-c2cccc(N(c3ccc(-c4cccc5ccccc45)cc3)c3ccc4c5ccccc5n(-c5cccc(-c6ccc7ccccc7c6)c5)c4c3)c21 ; c1cc(-c2ccc3ccccc3c2)cc(-n2c3ccccc3c3ccc(N(c4ccc(-c5cccc6ccccc56)cc4)c4cccc5sc6ccccc6c45)cc32)c1] 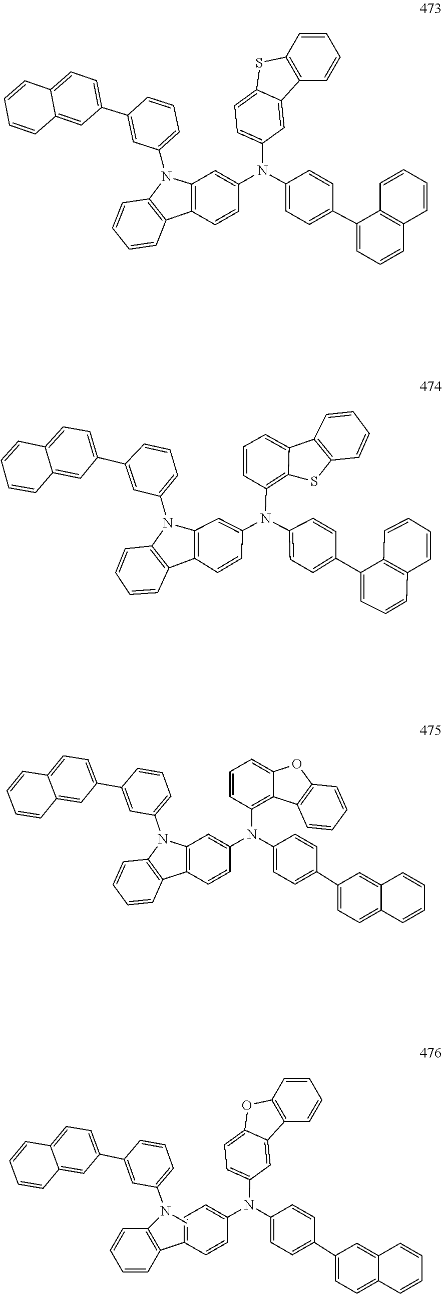 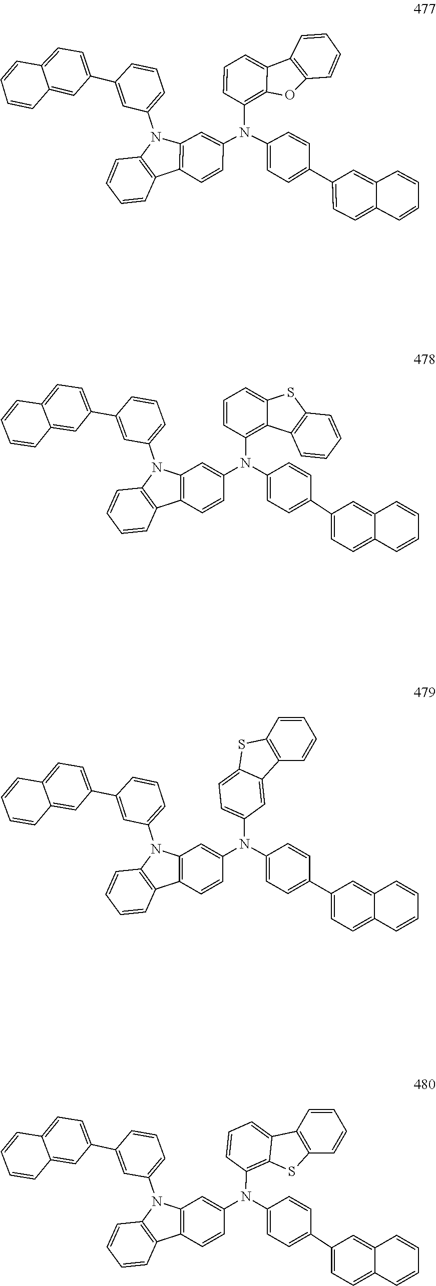 [SMILES: c1cc(-c2ccc3ccccc3c2)cc(-n2c3ccccc3c3ccc(N(c4ccc(-c5ccc6ccccc6c5)cc4)c4cccc5c4oc4ccccc45)cc32)c1 ; c1cc(-c2ccc3ccccc3c2)cc(-n2c3ccccc3c3ccc(N(c4ccc(-c5ccc6ccccc6c5)cc4)c4cccc5sc6ccccc6c45)cc32)c1 ; c1cc(-c2ccc3ccccc3c2)cc(-n2c3ccccc3c3ccc(N(c4ccc(-c5ccc6ccccc6c5)cc4)c4ccc5sc6ccccc6c5c4)cc32)c1 ; c1cc(-c2ccc3ccccc3c2)cc(-n2c3ccccc3c3ccc(N(c4ccc(-c5ccc6ccccc6c5)cc4)c4cccc5c4sc4ccccc45)cc32)c1] 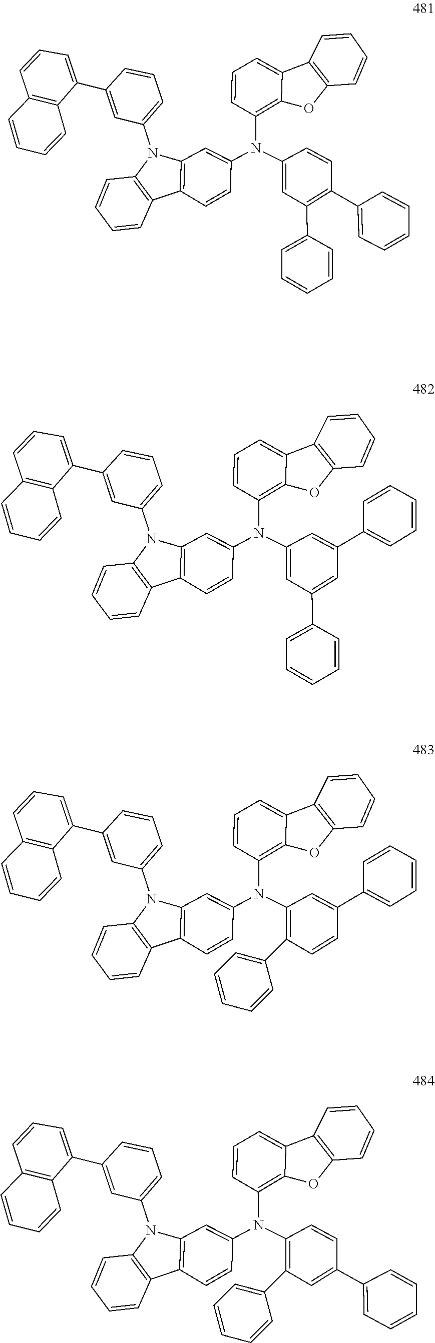 [SMILES: c1ccc(-c2ccc(N(c3ccc4c5ccccc5n(-c5cccc(-c6cccc7ccccc67)c5)c4c3)c3cccc4c3oc3ccccc34)cc2-c2ccccc2)cc1 ; c1ccc(-c2cc(-c3ccccc3)cc(N(c3ccc4c5ccccc5n(-c5cccc(-c6cccc7ccccc67)c5)c4c3)c3cccc4c3oc3ccccc34)c2)cc1 ; c1ccc(-c2ccc(-c3ccccc3)c(N(c3ccc4c5ccccc5n(-c5cccc(-c6cccc7ccccc67)c5)c4c3)c3cccc4c3oc3ccccc34)c2)cc1 ; c1ccc(-c2ccc(N(c3ccc4c5ccccc5n(-c5cccc(-c6cccc7ccccc67)c5)c4c3)c3cccc4c3oc3ccccc34)c(-c3ccccc3)c2)cc1] 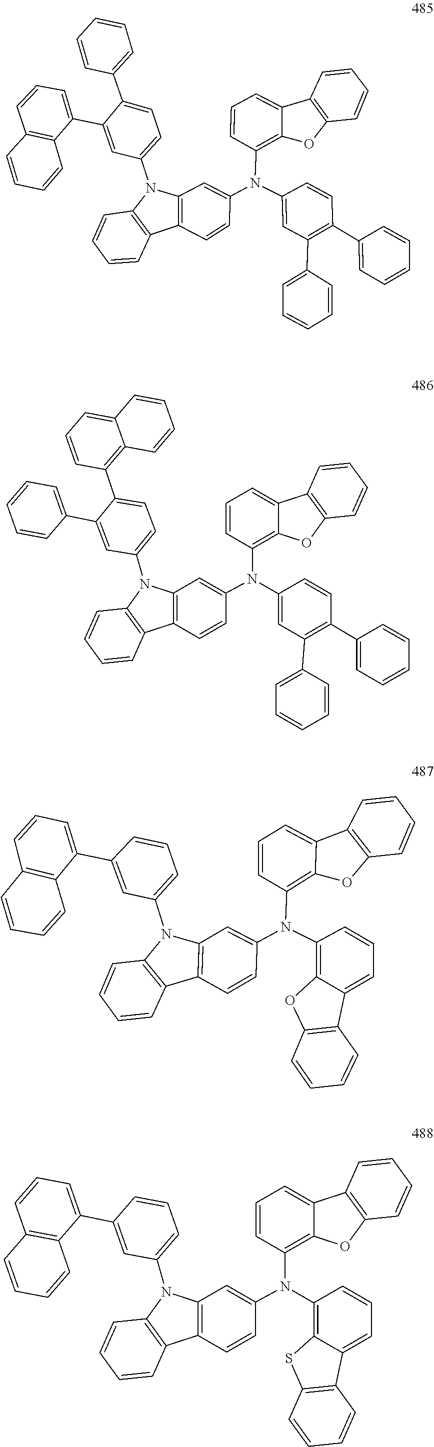 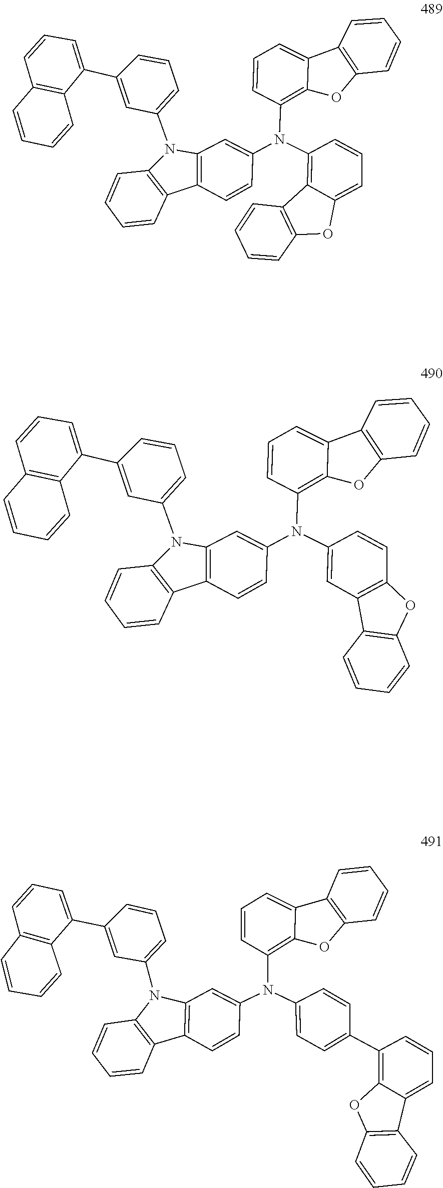 [SMILES: c1cc(-c2cccc3ccccc23)cc(-n2c3ccccc3c3ccc(N(c4cccc5c4oc4ccccc45)c4cccc5oc6ccccc6c45)cc32)c1 ; c1cc(-c2cccc3ccccc23)cc(-n2c3ccccc3c3ccc(N(c4ccc5oc6ccccc6c5c4)c4cccc5c4oc4ccccc45)cc32)c1 ; c1cc(-c2cccc3ccccc23)cc(-n2c3ccccc3c3ccc(N(c4ccc(-c5cccc6c5oc5ccccc56)cc4)c4cccc5c4oc4ccccc45)cc32)c1] 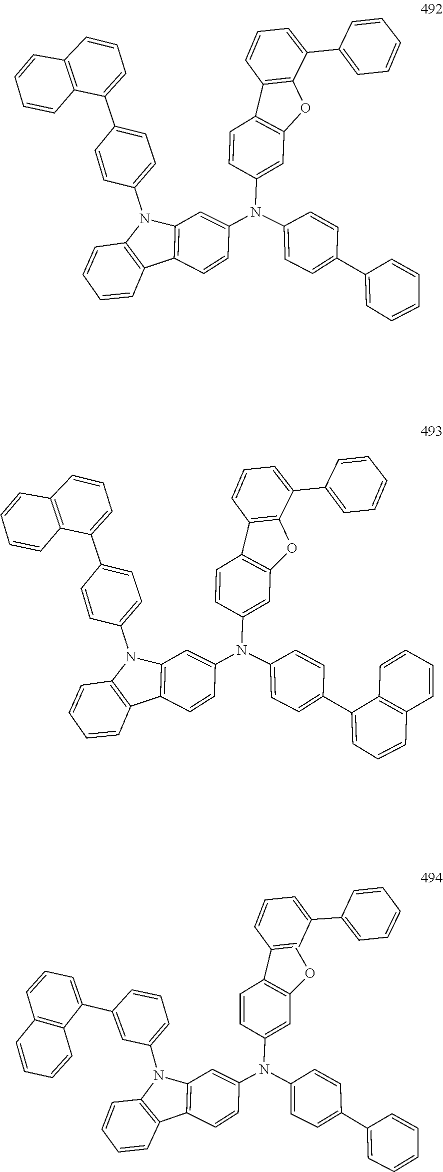 [SMILES: c1ccc(-c2ccc(N(c3ccc4c(c3)oc3c(-c5ccccc5)cccc34)c3ccc4c5ccccc5n(-c5ccc(-c6cccc7ccccc67)cc5)c4c3)cc2)cc1 ; c1ccc(-c2cccc3c2oc2cc(N(c4ccc(-c5cccc6ccccc56)cc4)c4ccc5c6ccccc6n(-c6ccc(-c7cccc8ccccc78)cc6)c5c4)ccc23)cc1 ; COc1cc(N(c2ccc(-c3ccccc3)cc2)c2ccc3c4ccccc4n(-c4cccc(-c5cccc6ccccc56)c4)c3c2)ccc1-c1cccc(-c2ccccc2)c1] 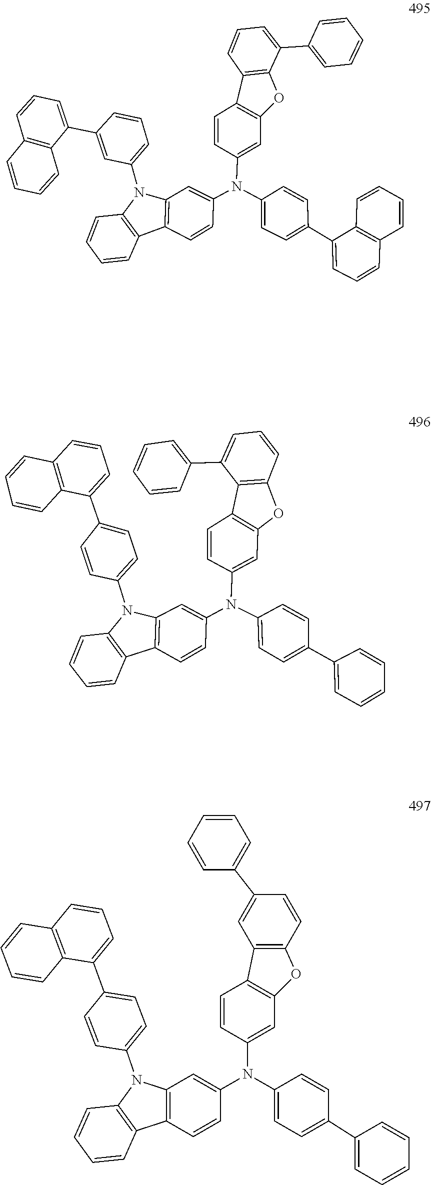 [SMILES: c1ccc(-c2cccc3c2oc2cc(N(c4ccc(-c5cccc6ccccc56)cc4)c4ccc5c6ccccc6n(-c6cccc(-c7cccc8ccccc78)c6)c5c4)ccc23)cc1 ; c1ccc(-c2ccc(N(c3ccc4c(c3)oc3cccc(-c5ccccc5)c34)c3ccc4c5ccccc5n(-c5ccc(-c6cccc7ccccc67)cc5)c4c3)cc2)cc1 ; c1ccc(-c2ccc(N(c3ccc4c(c3)oc3ccc(-c5ccccc5)cc34)c3ccc4c5ccccc5n(-c5ccc(-c6cccc7ccccc67)cc5)c4c3)cc2)cc1] 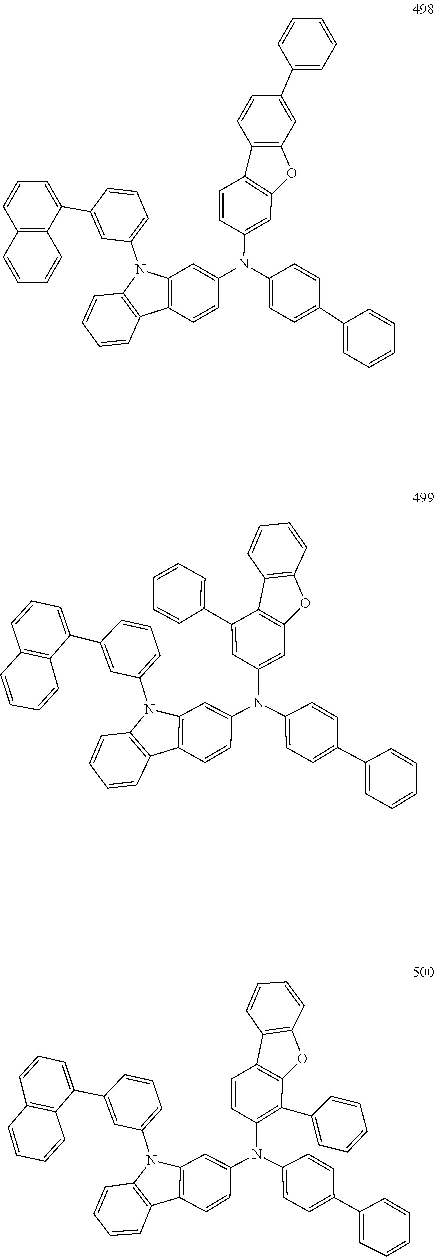 [SMILES: c1ccc(-c2ccc(N(c3ccc4c(c3)oc3cc(-c5ccccc5)ccc34)c3ccc4c5ccccc5n(-c5cccc(-c6cccc7ccccc67)c5)c4c3)cc2)cc1 ; c1ccc(-c2ccc(N(c3cc(-c4ccccc4)c4c(c3)oc3ccccc34)c3ccc4c5ccccc5n(-c5cccc(-c6cccc7ccccc67)c5)c4c3)cc2)cc1 ; c1ccc(-c2ccc(N(c3ccc4c5ccccc5n(-c5cccc(-c6cccc7ccccc67)c5)c4c3)c3ccc4c(oc5ccccc54)c3-c3ccccc3)cc2)cc1] 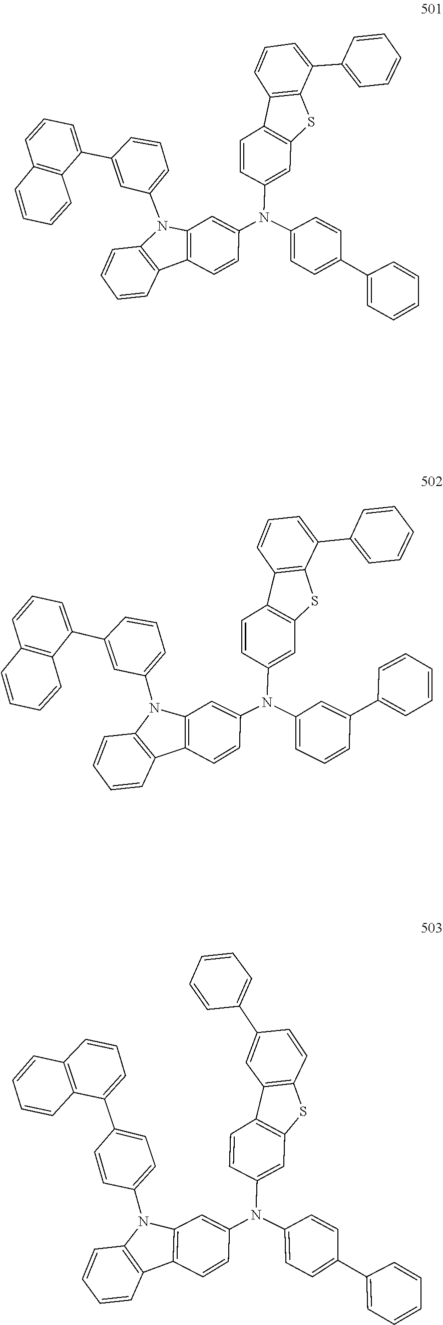 [SMILES: c1ccc(-c2ccc(N(c3ccc4c(c3)sc3c(-c5ccccc5)cccc34)c3ccc4c5ccccc5n(-c5cccc(-c6cccc7ccccc67)c5)c4c3)cc2)cc1 ; c1ccc(-c2cccc(N(c3ccc4c(c3)sc3c(-c5ccccc5)cccc34)c3ccc4c5ccccc5n(-c5cccc(-c6cccc7ccccc67)c5)c4c3)c2)cc1 ; c1ccc(-c2ccc(N(c3ccc4c(c3)sc3ccc(-c5ccccc5)cc34)c3ccc4c5ccccc5n(-c5ccc(-c6cccc7ccccc67)cc5)c4c3)cc2)cc1] 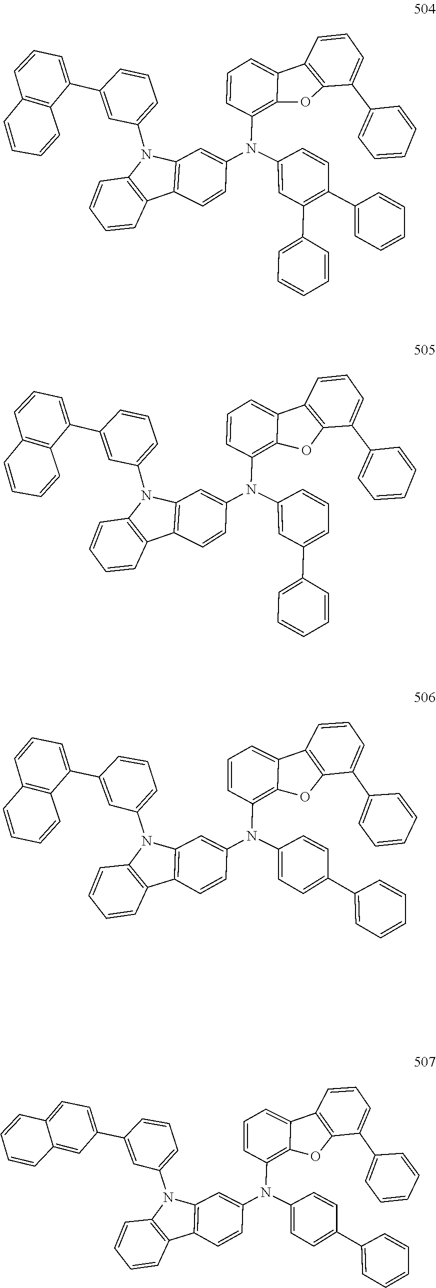 [SMILES: c1ccc(-c2ccc(N(c3ccc4c5ccccc5n(-c5cccc(-c6cccc7ccccc67)c5)c4c3)c3cccc4c3oc3c(-c5ccccc5)cccc34)cc2-c2ccccc2)cc1 ; c1ccc(-c2cccc(N(c3ccc4c5ccccc5n(-c5cccc(-c6cccc7ccccc67)c5)c4c3)c3cccc4c3oc3c(-c5ccccc5)cccc34)c2)cc1 ; c1ccc(-c2ccc(N(c3ccc4c5ccccc5n(-c5cccc(-c6cccc7ccccc67)c5)c4c3)c3cccc4c3oc3c(-c5ccccc5)cccc34)cc2)cc1 ; c1ccc(-c2ccc(N(c3ccc4c5ccccc5n(-c5cccc(-c6ccc7ccccc7c6)c5)c4c3)c3cccc4c3oc3c(-c5ccccc5)cccc34)cc2)cc1] 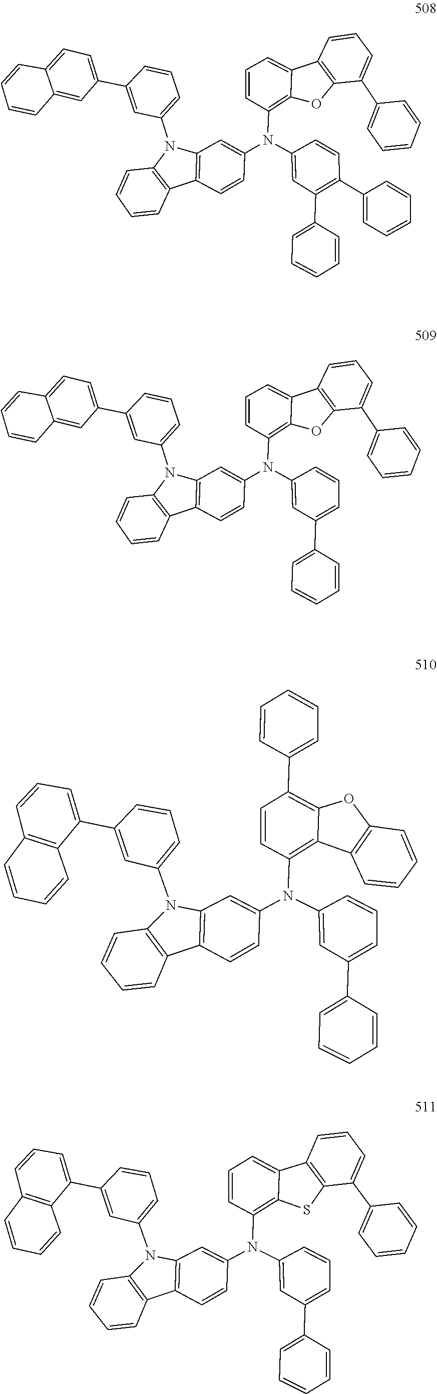 [SMILES: c1ccc(-c2ccc(N(c3ccc4c5ccccc5n(-c5cccc(-c6ccc7ccccc7c6)c5)c4c3)c3cccc4c3oc3c(-c5ccccc5)cccc34)cc2-c2ccccc2)cc1 ; c1ccc(-c2cccc(N(c3ccc4c5ccccc5n(-c5cccc(-c6ccc7ccccc7c6)c5)c4c3)c3cccc4c3oc3c(-c5ccccc5)cccc34)c2)cc1 ; c1ccc(-c2cccc(N(c3ccc4c5ccccc5n(-c5cccc(-c6cccc7ccccc67)c5)c4c3)c3ccc(-c4ccccc4)c4oc5ccccc5c34)c2)cc1 ; c1ccc(-c2cccc(N(c3ccc4c5ccccc5n(-c5cccc(-c6cccc7ccccc67)c5)c4c3)c3cccc4c3sc3c(-c5ccccc5)cccc34)c2)cc1] 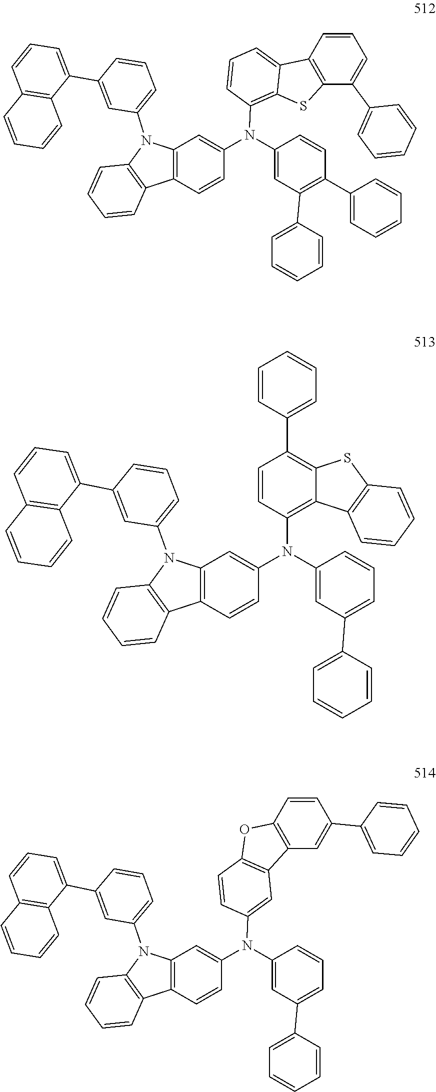 [SMILES: c1ccc(-c2ccc(N(c3ccc4c5ccccc5n(-c5cccc(-c6cccc7ccccc67)c5)c4c3)c3cccc4c3sc3c(-c5ccccc5)cccc34)cc2-c2ccccc2)cc1 ; c1ccc(-c2cccc(N(c3ccc4c5ccccc5n(-c5cccc(-c6cccc7ccccc67)c5)c4c3)c3ccc(-c4ccccc4)c4sc5ccccc5c34)c2)cc1 ; c1ccc(-c2cccc(N(c3ccc4oc5ccc(-c6ccccc6)cc5c4c3)c3ccc4c5ccccc5n(-c5cccc(-c6cccc7ccccc67)c5)c4c3)c2)cc1] 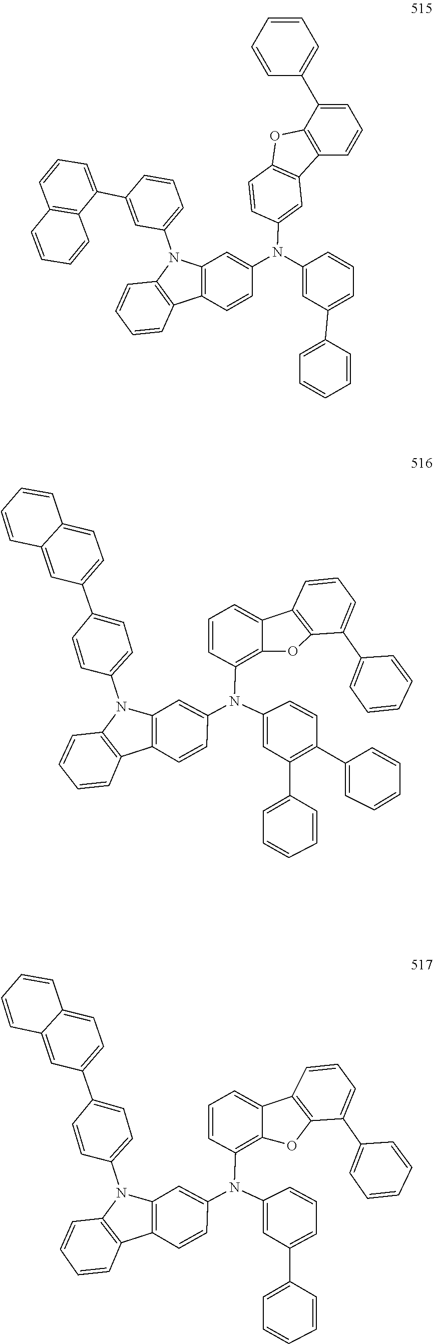 [SMILES: c1ccc(-c2cccc(N(c3ccc4oc5c(-c6ccccc6)cccc5c4c3)c3ccc4c5ccccc5n(-c5cccc(-c6cccc7ccccc67)c5)c4c3)c2)cc1 ; c1ccc(-c2ccc(N(c3ccc4c5ccccc5n(-c5ccc(-c6ccc7ccccc7c6)cc5)c4c3)c3cccc4c3oc3c(-c5ccccc5)cccc34)cc2-c2ccccc2)cc1 ; c1ccc(-c2cccc(N(c3ccc4c5ccccc5n(-c5ccc(-c6ccc7ccccc7c6)cc5)c4c3)c3cccc4c3oc3c(-c5ccccc5)cccc34)c2)cc1] 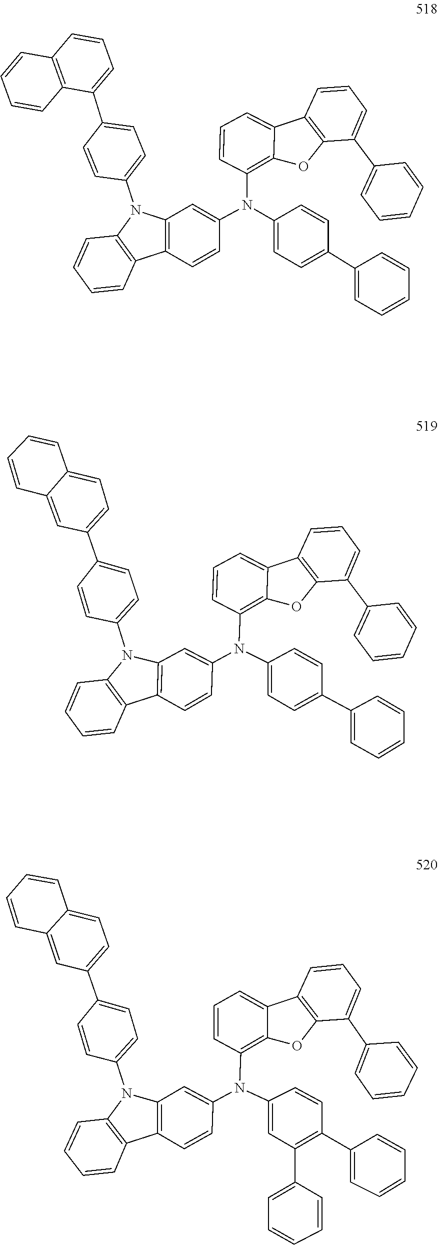 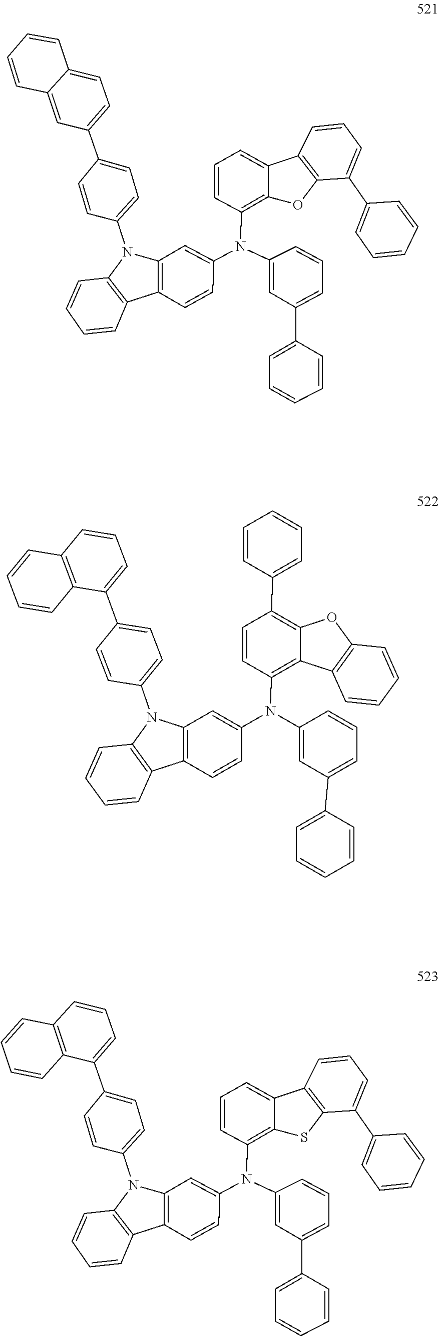 [SMILES: c1ccc(-c2cccc(N(c3ccc4c5ccccc5n(-c5ccc(-c6ccc7ccccc7c6)cc5)c4c3)c3cccc4c3oc3c(-c5ccccc5)cccc34)c2)cc1 ; c1ccc(-c2cccc(N(c3ccc4c5ccccc5n(-c5ccc(-c6cccc7ccccc67)cc5)c4c3)c3ccc(-c4ccccc4)c4oc5ccccc5c34)c2)cc1 ; c1ccc(-c2cccc(N(c3ccc4c5ccccc5n(-c5ccc(-c6cccc7ccccc67)cc5)c4c3)c3cccc4c3sc3c(-c5ccccc5)cccc34)c2)cc1] 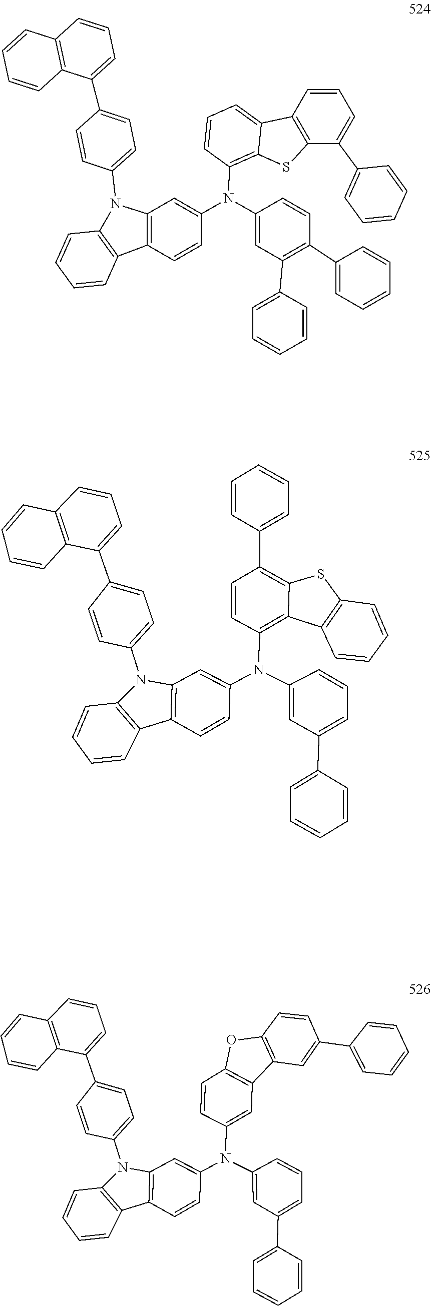 [SMILES: c1ccc(-c2ccc(N(c3ccc4c5ccccc5n(-c5ccc(-c6cccc7ccccc67)cc5)c4c3)c3cccc4c3sc3c(-c5ccccc5)cccc34)cc2-c2ccccc2)cc1 ; c1ccc(-c2cccc(N(c3ccc4c5ccccc5n(-c5ccc(-c6cccc7ccccc67)cc5)c4c3)c3ccc(-c4ccccc4)c4sc5ccccc5c34)c2)cc1 ; c1ccc(-c2cccc(N(c3ccc4oc5ccc(-c6ccccc6)cc5c4c3)c3ccc4c5ccccc5n(-c5ccc(-c6cccc7ccccc67)cc5)c4c3)c2)cc1] 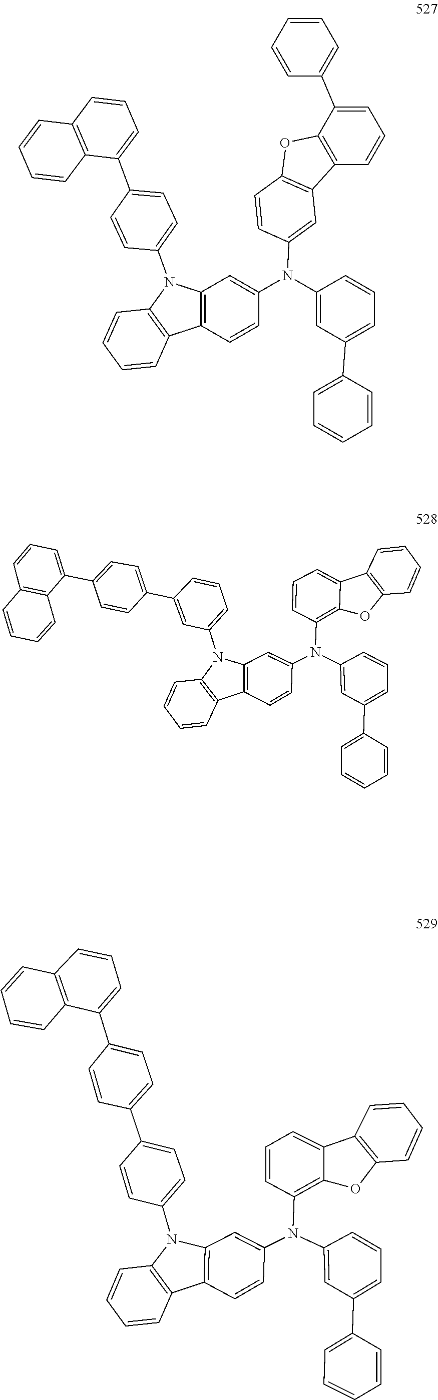 [SMILES: c1ccc(-c2cccc(N(c3ccc4oc5c(-c6ccccc6)cccc5c4c3)c3ccc4c5ccccc5n(-c5ccc(-c6cccc7ccccc67)cc5)c4c3)c2)cc1 ; c1ccc(-c2cccc(N(c3ccc4c5ccccc5n(-c5cccc(-c6ccc(-c7cccc8ccccc78)cc6)c5)c4c3)c3cccc4c3oc3ccccc34)c2)cc1 ; c1ccc(-c2cccc(N(c3ccc4c5ccccc5n(-c5ccc(-c6ccc(-c7cccc8ccccc78)cc6)cc5)c4c3)c3cccc4c3oc3ccccc34)c2)cc1] 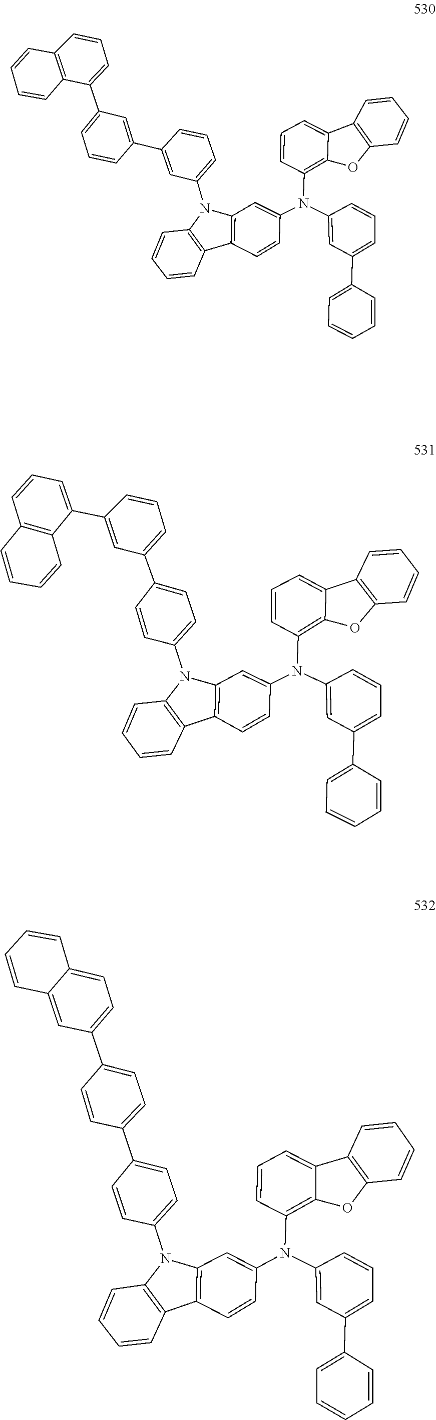 [SMILES: c1ccc(-c2cccc(N(c3ccc4c5ccccc5n(-c5cccc(-c6cccc(-c7cccc8ccccc78)c6)c5)c4c3)c3cccc4c3oc3ccccc34)c2)cc1 ; c1ccc(-c2cccc(N(c3ccc4c5ccccc5n(-c5ccc(-c6cccc(-c7cccc8ccccc78)c6)cc5)c4c3)c3cccc4c3oc3ccccc34)c2)cc1 ; c1ccc(-c2cccc(N(c3ccc4c5ccccc5n(-c5ccc(-c6ccc(-c7ccc8ccccc8c7)cc6)cc5)c4c3)c3cccc4c3oc3ccccc34)c2)cc1] 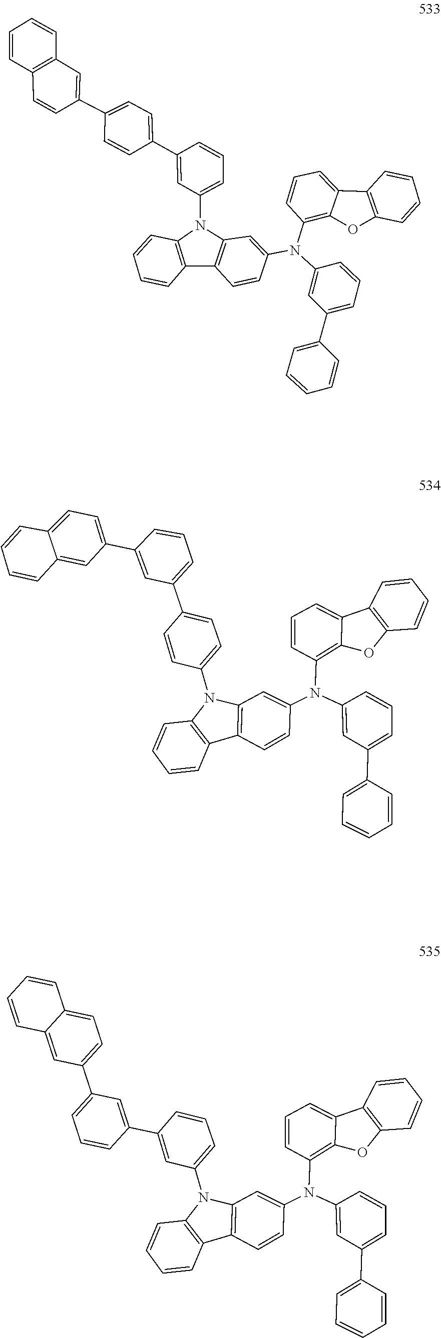 [SMILES: c1ccc(-c2cccc(N(c3ccc4c5ccccc5n(-c5cccc(-c6ccc(-c7ccc8ccccc8c7)cc6)c5)c4c3)c3cccc4c3oc3ccccc34)c2)cc1 ; c1ccc(-c2cccc(N(c3ccc4c5ccccc5n(-c5ccc(-c6cccc(-c7ccc8ccccc8c7)c6)cc5)c4c3)c3cccc4c3oc3ccccc34)c2)cc1 ; c1ccc(-c2cccc(N(c3ccc4c5ccccc5n(-c5cccc(-c6cccc(-c7ccc8ccccc8c7)c6)c5)c4c3)c3cccc4c3oc3ccccc34)c2)cc1] 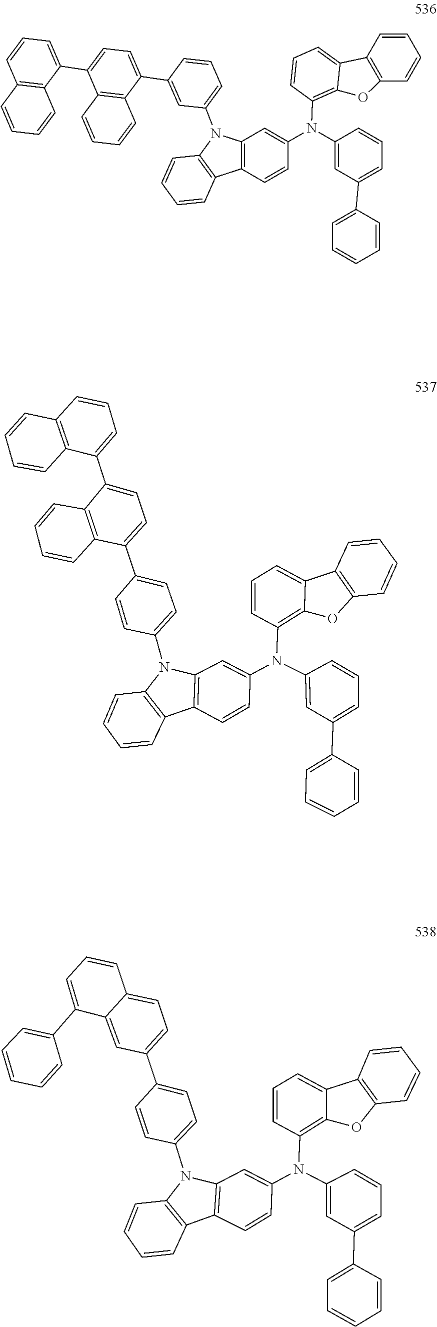 [SMILES: c1ccc(-c2cccc(N(c3ccc4c5ccccc5n(-c5cccc(-c6ccc(-c7cccc8ccccc78)c7ccccc67)c5)c4c3)c3cccc4c3oc3ccccc34)c2)cc1 ; c1ccc(-c2cccc(N(c3ccc4c5ccccc5n(-c5ccc(-c6ccc(-c7cccc8ccccc78)c7ccccc67)cc5)c4c3)c3cccc4c3oc3ccccc34)c2)cc1 ; c1ccc(-c2cccc(N(c3ccc4c5ccccc5n(-c5ccc(-c6ccc7cccc(-c8ccccc8)c7c6)cc5)c4c3)c3cccc4c3oc3ccccc34)c2)cc1] 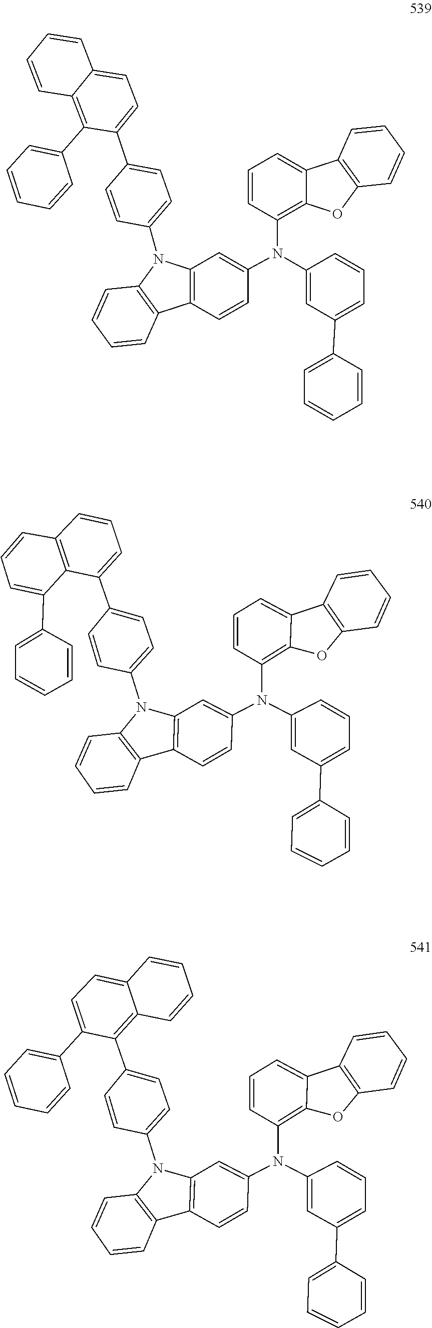 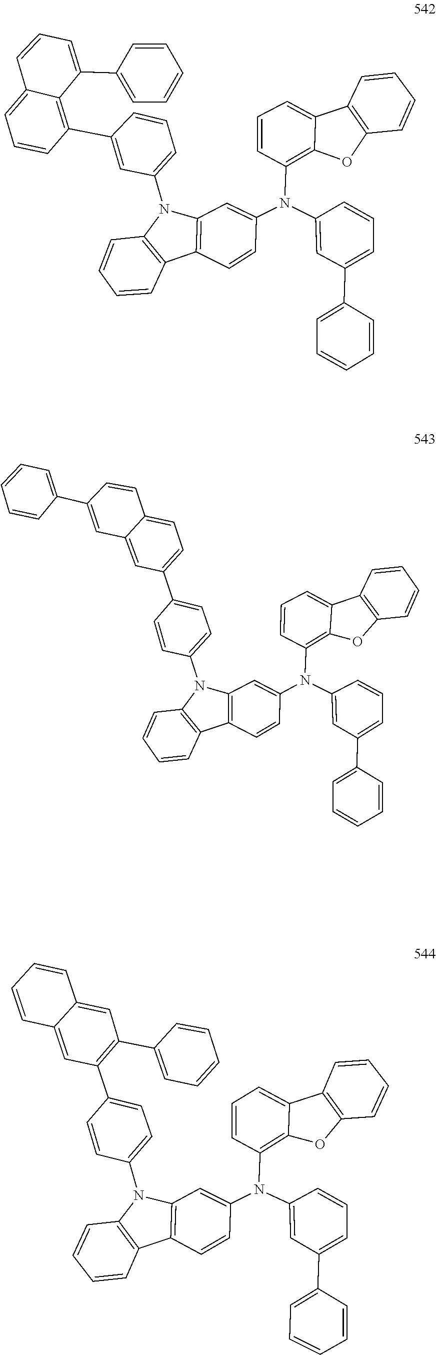 [SMILES: c1ccc(-c2cccc(N(c3ccc4c5ccccc5n(-c5cccc(-c6cccc7cccc(-c8ccccc8)c67)c5)c4c3)c3cccc4c3oc3ccccc34)c2)cc1 ; c1ccc(-c2cccc(N(c3ccc4c5ccccc5n(-c5ccc(-c6ccc7ccc(-c8ccccc8)cc7c6)cc5)c4c3)c3cccc4c3oc3ccccc34)c2)cc1 ; c1ccc(-c2cccc(N(c3ccc4c5ccccc5n(-c5ccc(-c6cc7ccccc7cc6-c6ccccc6)cc5)c4c3)c3cccc4c3oc3ccccc34)c2)cc1] 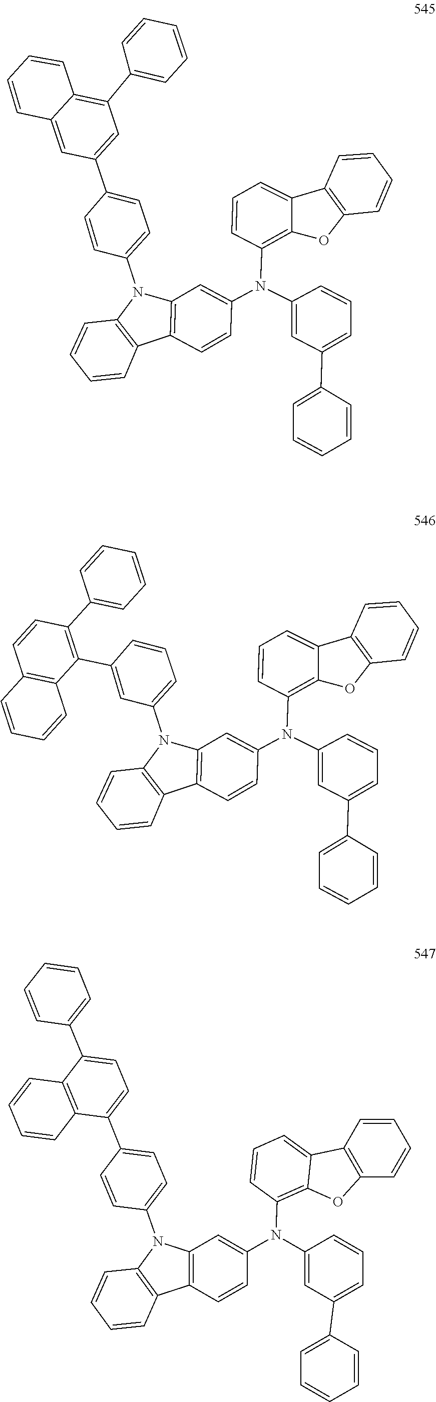 [SMILES: c1ccc(-c2cccc(N(c3ccc4c5ccccc5n(-c5ccc(-c6cc(-c7ccccc7)c7ccccc7c6)cc5)c4c3)c3cccc4c3oc3ccccc34)c2)cc1 ; c1ccc(-c2cccc(N(c3ccc4c5ccccc5n(-c5cccc(-c6c(-c7ccccc7)ccc7ccccc67)c5)c4c3)c3cccc4c3oc3ccccc34)c2)cc1 ; c1ccc(-c2cccc(N(c3ccc4c5ccccc5n(-c5ccc(-c6ccc(-c7ccccc7)c7ccccc67)cc5)c4c3)c3cccc4c3oc3ccccc34)c2)cc1] 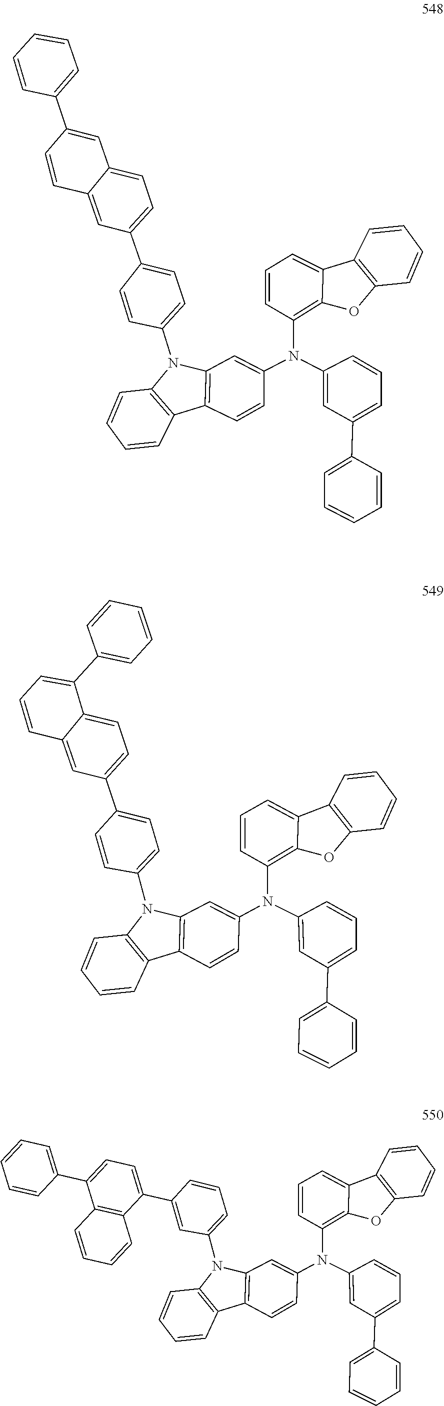 [SMILES: c1ccc(-c2cccc(N(c3ccc4c5ccccc5n(-c5ccc(-c6ccc7cc(-c8ccccc8)ccc7c6)cc5)c4c3)c3cccc4c3oc3ccccc34)c2)cc1 ; c1ccc(-c2cccc(N(c3ccc4c5ccccc5n(-c5ccc(-c6ccc7c(-c8ccccc8)cccc7c6)cc5)c4c3)c3cccc4c3oc3ccccc34)c2)cc1 ; c1ccc(-c2cccc(N(c3ccc4c5ccccc5n(-c5cccc(-c6ccc(-c7ccccc7)c7ccccc67)c5)c4c3)c3cccc4c3oc3ccccc34)c2)cc1] 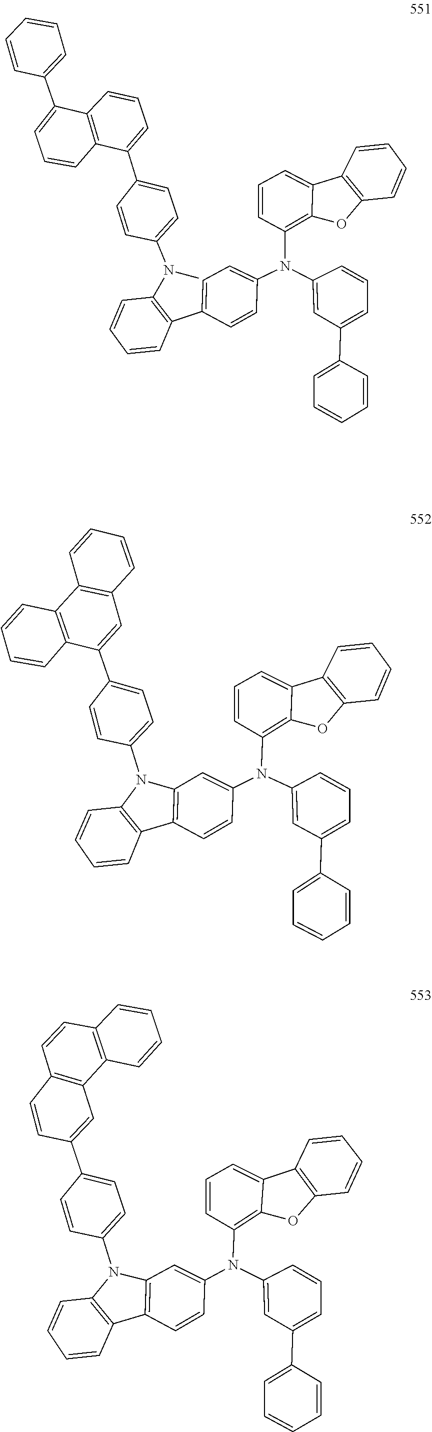 [SMILES: CN(c1ccc(-c2cccc3c(-c4ccccc4)cccc23)cc1)c1ccccc1-c1ccc(N(c2cccc(-c3ccccc3)c2)c2cccc3c2oc2ccccc23)cc1 ; c1ccc(-c2cccc(N(c3ccc4c5ccccc5n(-c5ccc(-c6cc7ccccc7c7ccccc67)cc5)c4c3)c3cccc4c3oc3ccccc34)c2)cc1 ; c1ccc(-c2cccc(N(c3ccc4c5ccccc5n(-c5ccc(-c6ccc7ccc8ccccc8c7c6)cc5)c4c3)c3cccc4c3oc3ccccc34)c2)cc1] 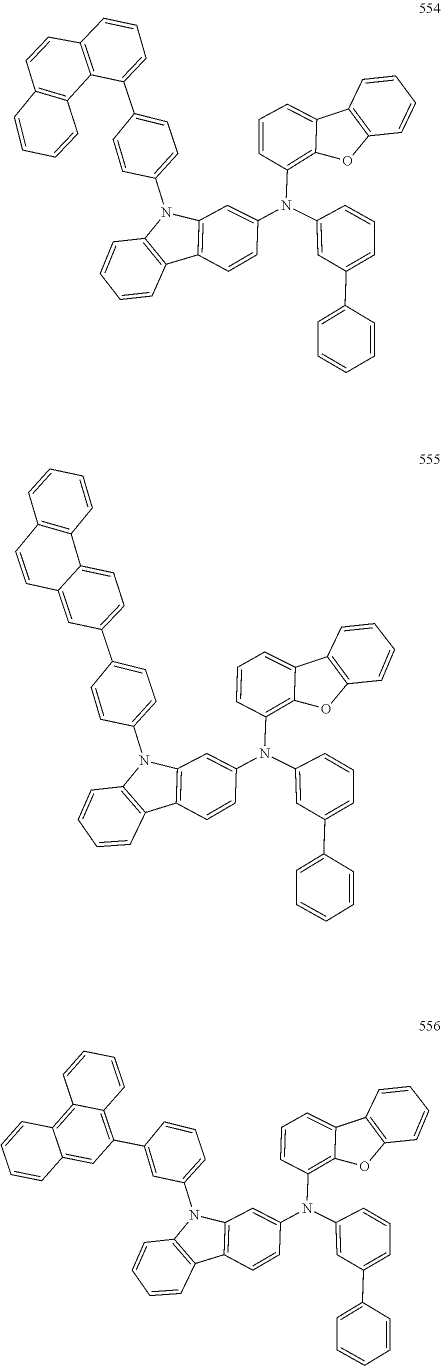 [SMILES: c1ccc(-c2cccc(N(c3ccc4c5ccccc5n(-c5ccc(-c6cccc7ccc8ccccc8c67)cc5)c4c3)c3cccc4c3oc3ccccc34)c2)cc1 ; c1ccc(-c2cccc(N(c3ccc4c5ccccc5n(-c5ccc(-c6ccc7c(ccc8ccccc87)c6)cc5)c4c3)c3cccc4c3oc3ccccc34)c2)cc1 ; c1ccc(-c2cccc(N(c3ccc4c5ccccc5n(-c5cccc(-c6cc7ccccc7c7ccccc67)c5)c4c3)c3cccc4c3oc3ccccc34)c2)cc1] 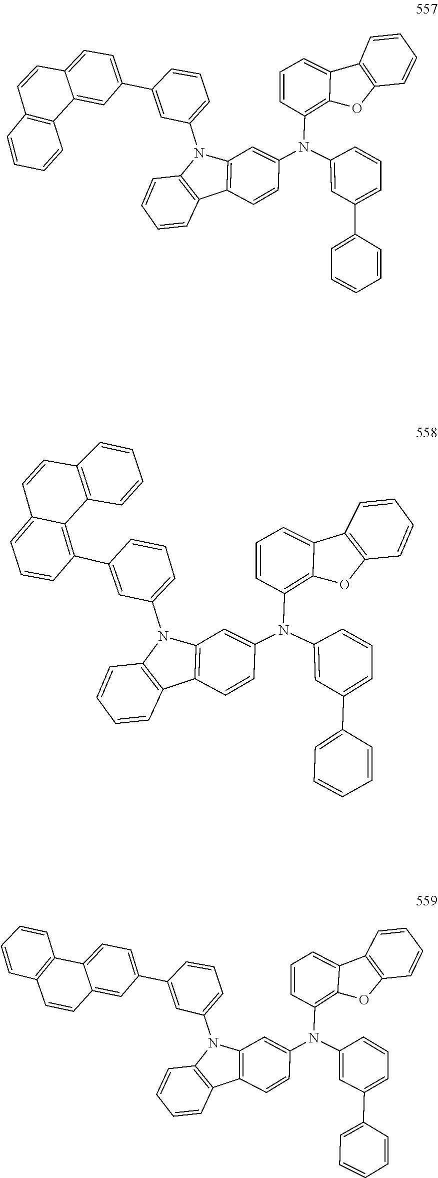 [SMILES: c1ccc(-c2cccc(N(c3ccc4c5ccccc5n(-c5cccc(-c6ccc7ccc8ccccc8c7c6)c5)c4c3)c3cccc4c3oc3ccccc34)c2)cc1 ; c1ccc(-c2cccc(N(c3ccc4c5ccccc5n(-c5cccc(-c6cccc7ccc8ccccc8c67)c5)c4c3)c3cccc4c3oc3ccccc34)c2)cc1 ; c1ccc(-c2cccc(N(c3ccc4c5ccccc5n(-c5cccc(-c6ccc7c(ccc8ccccc87)c6)c5)c4c3)c3cccc4c3oc3ccccc34)c2)cc1] 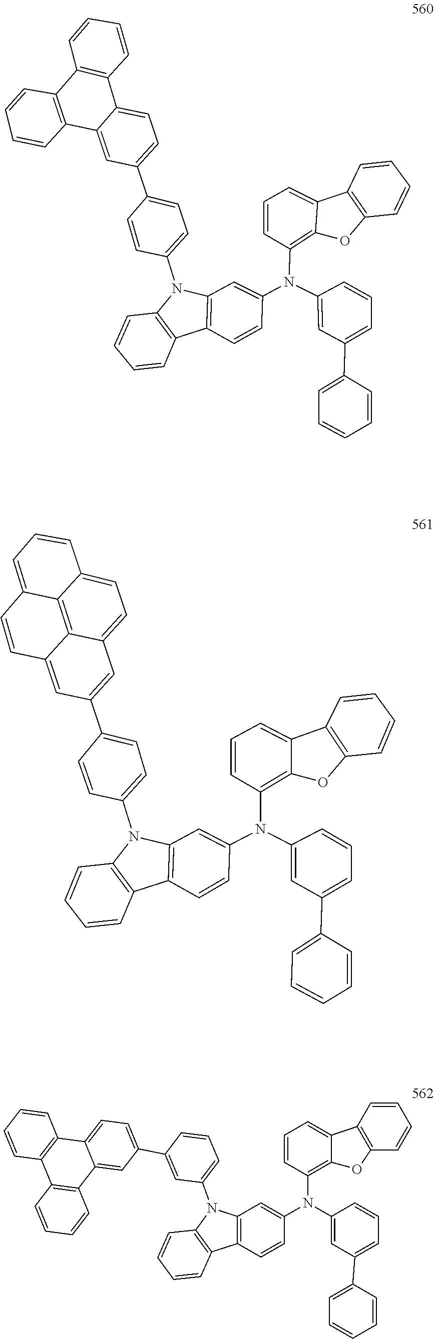 [SMILES: c1ccc(-c2cccc(N(c3ccc4c5ccccc5n(-c5ccc(-c6ccc7c8ccccc8c8ccccc8c7c6)cc5)c4c3)c3cccc4c3oc3ccccc34)c2)cc1 ; c1ccc(-c2cccc(N(c3ccc4c5ccccc5n(-c5ccc(-c6cc7ccc8cccc9ccc(c6)c7c89)cc5)c4c3)c3cccc4c3oc3ccccc34)c2)cc1 ; c1ccc(-c2cccc(N(c3ccc4c5ccccc5n(-c5cccc(-c6ccc7c8ccccc8c8ccccc8c7c6)c5)c4c3)c3cccc4c3oc3ccccc34)c2)cc1] 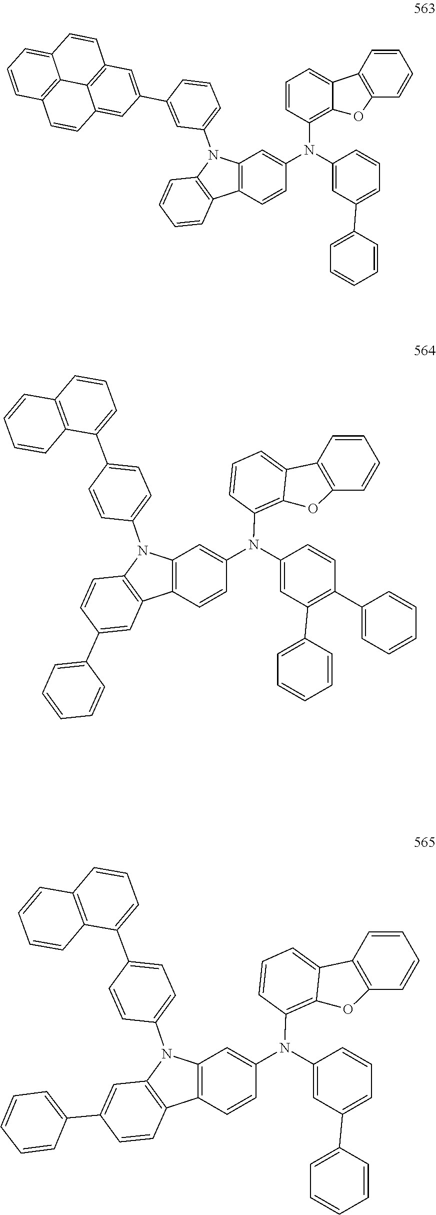 [SMILES: c1ccc(-c2cccc(N(c3ccc4c5ccccc5n(-c5cccc(-c6cc7ccc8cccc9ccc(c6)c7c89)c5)c4c3)c3cccc4c3oc3ccccc34)c2)cc1 ; c1ccc(-c2ccc3c(c2)c2ccc(N(c4ccc(-c5ccccc5)c(-c5ccccc5)c4)c4cccc5c4oc4ccccc45)cc2n3-c2ccc(-c3cccc4ccccc34)cc2)cc1 ; c1ccc(-c2cccc(N(c3ccc4c5ccc(-c6ccccc6)cc5n(-c5ccc(-c6cccc7ccccc67)cc5)c4c3)c3cccc4c3oc3ccccc34)c2)cc1] 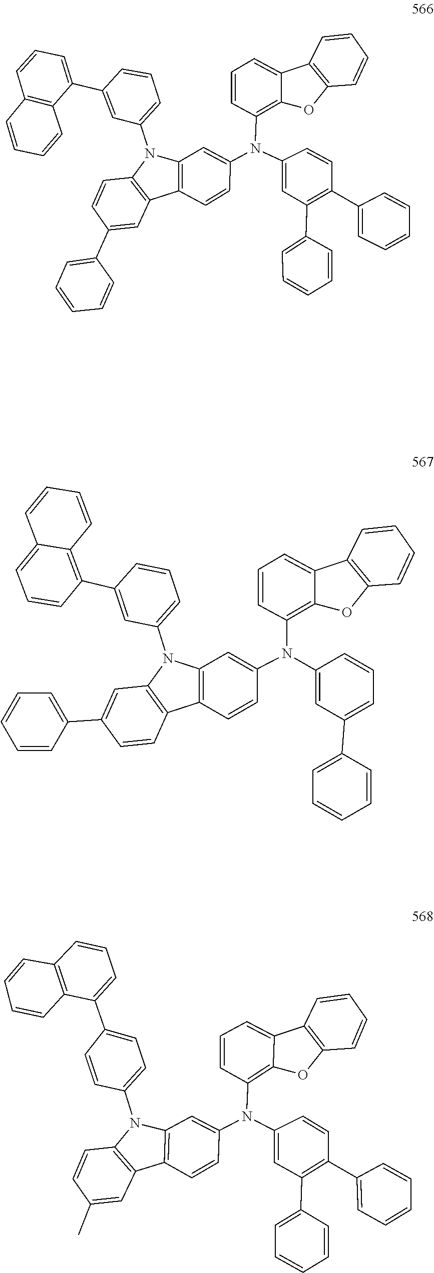 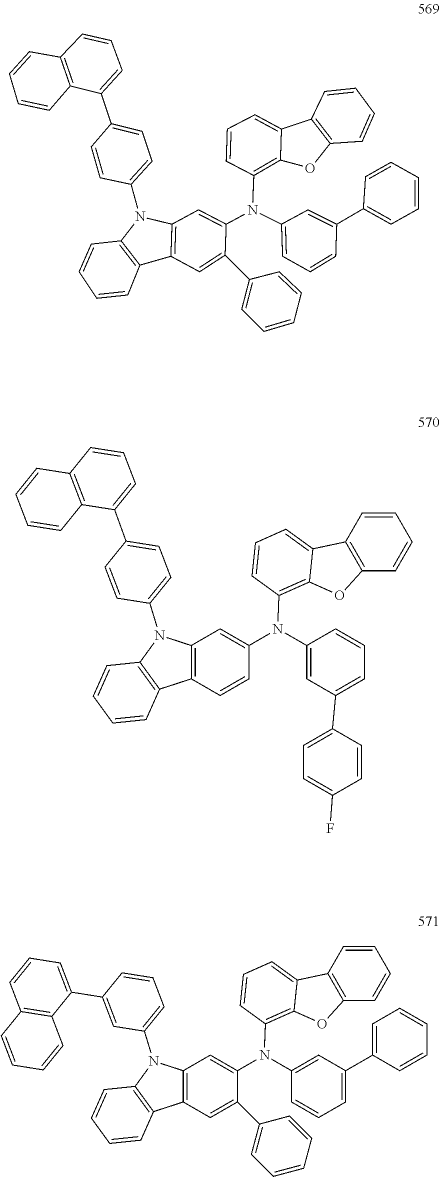 [SMILES: c1ccc(-c2cccc(N(c3cc4c(cc3-c3ccccc3)c3ccccc3n4-c3ccc(-c4cccc5ccccc45)cc3)c3cccc4c3oc3ccccc34)c2)cc1 ; Fc1ccc(-c2cccc(N(c3ccc4c5ccccc5n(-c5ccc(-c6cccc7ccccc67)cc5)c4c3)c3cccc4c3oc3ccccc34)c2)cc1 ; c1ccc(-c2cccc(N(c3cc4c(cc3-c3ccccc3)c3ccccc3n4-c3cccc(-c4cccc5ccccc45)c3)c3cccc4c3oc3ccccc34)c2)cc1] 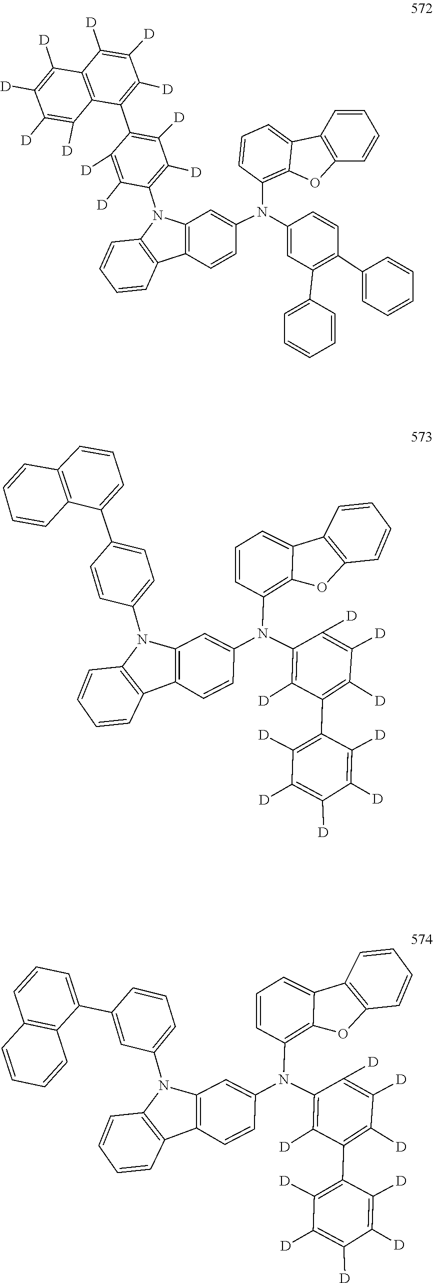 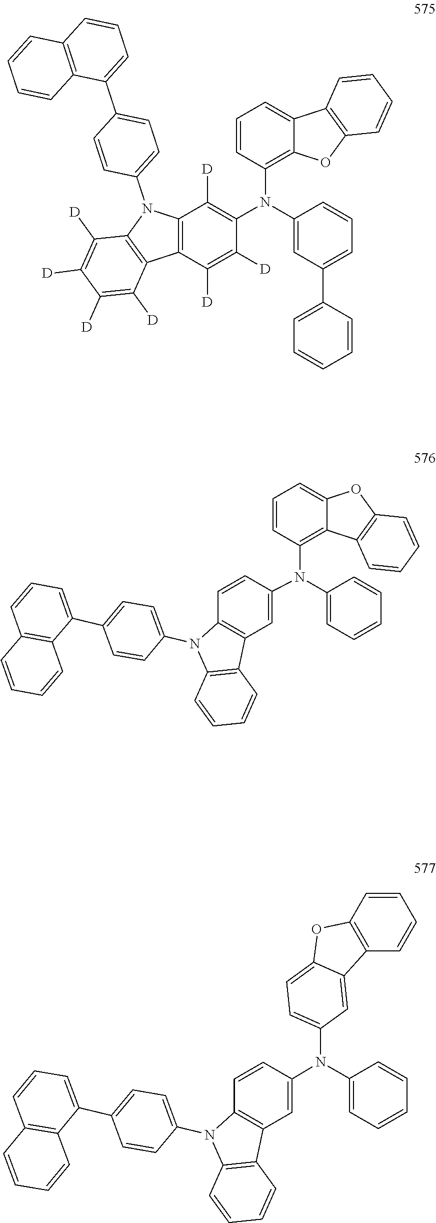 [SMILES: [2H]c1c([2H])c([2H])c2c(c1[2H])c1c([2H])c([2H])c(N(c3cccc(-c4ccccc4)c3)c3cccc4c3oc3ccccc34)c([2H])c1n2-c1ccc(-c2cccc3ccccc23)cc1 ; c1ccc(N(c2ccc3c(c2)c2ccccc2n3-c2ccc(-c3cccc4ccccc34)cc2)c2cccc3oc4ccccc4c23)cc1 ; c1ccc(N(c2ccc3oc4ccccc4c3c2)c2ccc3c(c2)c2ccccc2n3-c2ccc(-c3cccc4ccccc34)cc2)cc1] 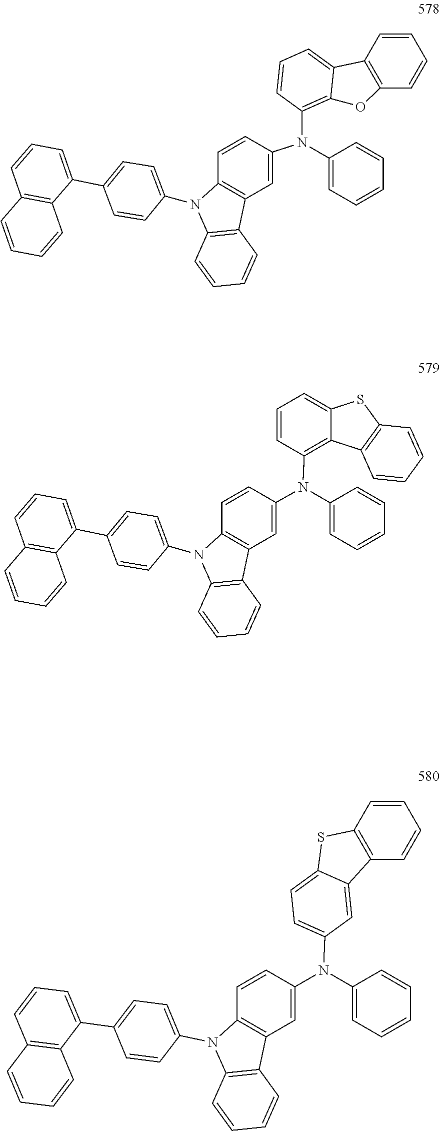 [SMILES: c1ccc(N(c2ccc3c(c2)c2ccccc2n3-c2ccc(-c3cccc4ccccc34)cc2)c2cccc3c2oc2ccccc23)cc1 ; c1ccc(N(c2ccc3c(c2)c2ccccc2n3-c2ccc(-c3cccc4ccccc34)cc2)c2cccc3sc4ccccc4c23)cc1 ; c1ccc(N(c2ccc3sc4ccccc4c3c2)c2ccc3c(c2)c2ccccc2n3-c2ccc(-c3cccc4ccccc34)cc2)cc1] 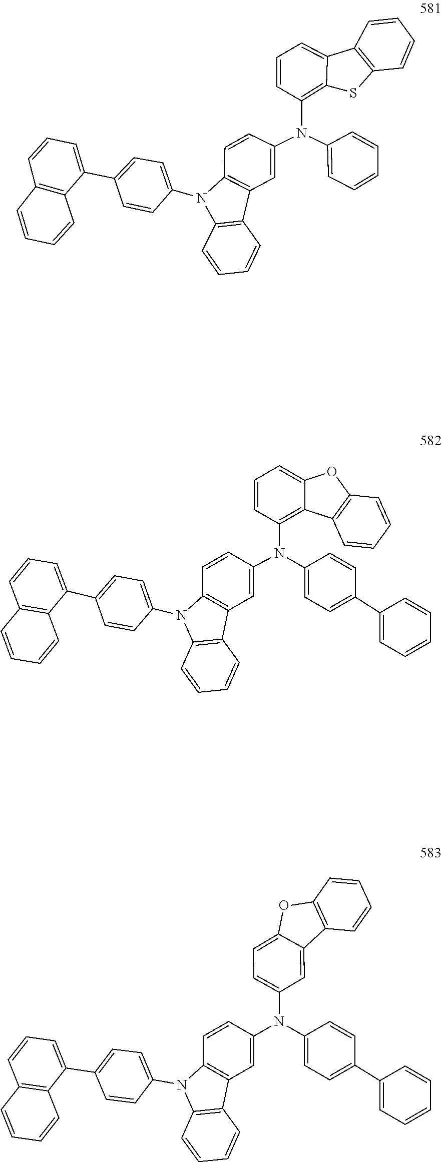 [SMILES: c1ccc(N(c2ccc3c(c2)c2ccccc2n3-c2ccc(-c3cccc4ccccc34)cc2)c2cccc3c2sc2ccccc23)cc1 ; c1ccc(-c2ccc(N(c3ccc4c(c3)c3ccccc3n4-c3ccc(-c4cccc5ccccc45)cc3)c3cccc4oc5ccccc5c34)cc2)cc1 ; c1ccc(-c2ccc(N(c3ccc4oc5ccccc5c4c3)c3ccc4c(c3)c3ccccc3n4-c3ccc(-c4cccc5ccccc45)cc3)cc2)cc1] 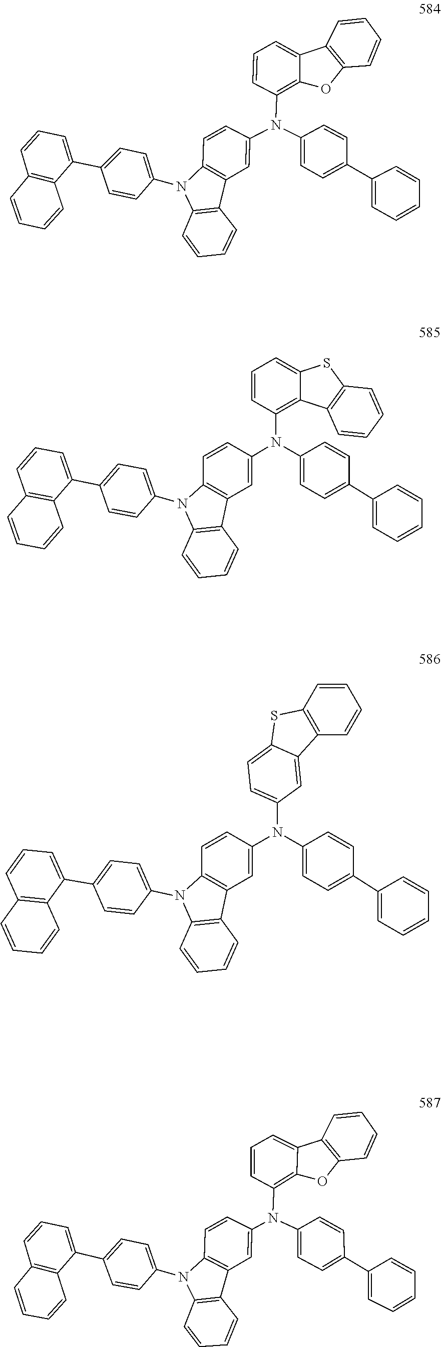 [SMILES: c1ccc(-c2ccc(N(c3ccc4c(c3)c3ccccc3n4-c3ccc(-c4cccc5ccccc45)cc3)c3cccc4c3oc3ccccc34)cc2)cc1 ; c1ccc(-c2ccc(N(c3ccc4c(c3)c3ccccc3n4-c3ccc(-c4cccc5ccccc45)cc3)c3cccc4sc5ccccc5c34)cc2)cc1 ; c1ccc(-c2ccc(N(c3ccc4sc5ccccc5c4c3)c3ccc4c(c3)c3ccccc3n4-c3ccc(-c4cccc5ccccc45)cc3)cc2)cc1 ; c1ccc(-c2ccc(N(c3ccc4c(c3)c3ccccc3n4-c3ccc(-c4cccc5ccccc45)cc3)c3cccc4c3oc3ccccc34)cc2)cc1] 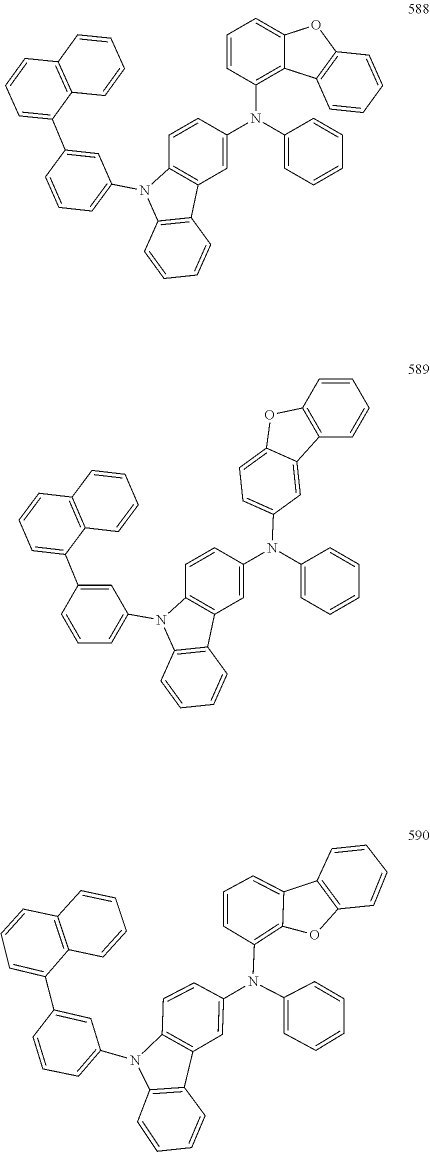 [SMILES: c1ccc(N(c2ccc3c(c2)c2ccccc2n3-c2cccc(-c3cccc4ccccc34)c2)c2cccc3oc4ccccc4c23)cc1 ; c1ccc(N(c2ccc3oc4ccccc4c3c2)c2ccc3c(c2)c2ccccc2n3-c2cccc(-c3cccc4ccccc34)c2)cc1 ; c1ccc(N(c2ccc3c(c2)c2ccccc2n3-c2cccc(-c3cccc4ccccc34)c2)c2cccc3c2oc2ccccc23)cc1] 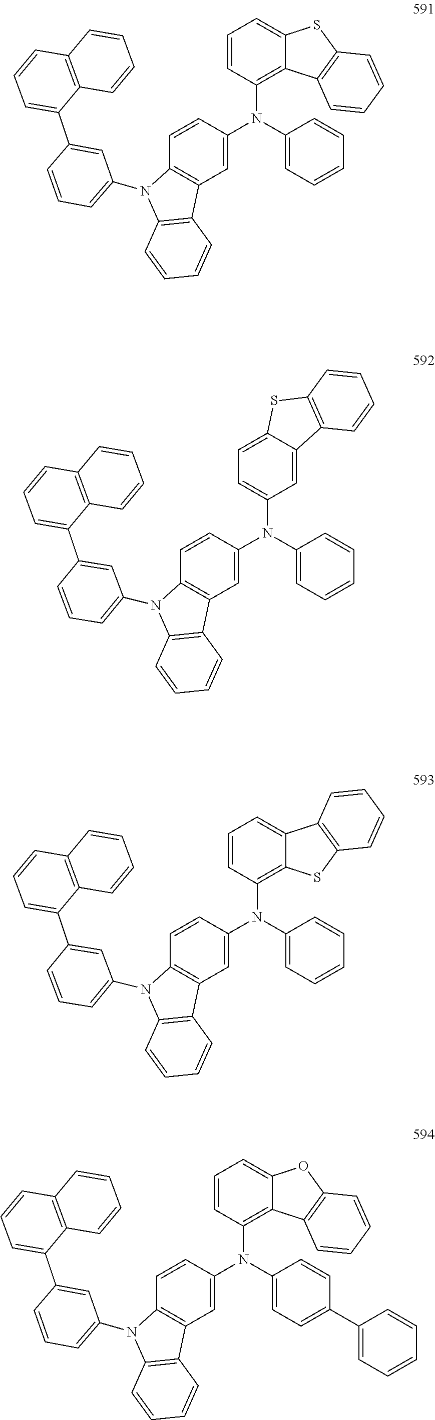 [SMILES: c1ccc(N(c2ccc3c(c2)c2ccccc2n3-c2cccc(-c3cccc4ccccc34)c2)c2cccc3sc4ccccc4c23)cc1 ; c1ccc(N(c2ccc3sc4ccccc4c3c2)c2ccc3c(c2)c2ccccc2n3-c2cccc(-c3cccc4ccccc34)c2)cc1 ; c1ccc(N(c2ccc3c(c2)c2ccccc2n3-c2cccc(-c3cccc4ccccc34)c2)c2cccc3c2sc2ccccc23)cc1 ; c1ccc(-c2ccc(N(c3ccc4c(c3)c3ccccc3n4-c3cccc(-c4cccc5ccccc45)c3)c3cccc4oc5ccccc5c34)cc2)cc1] 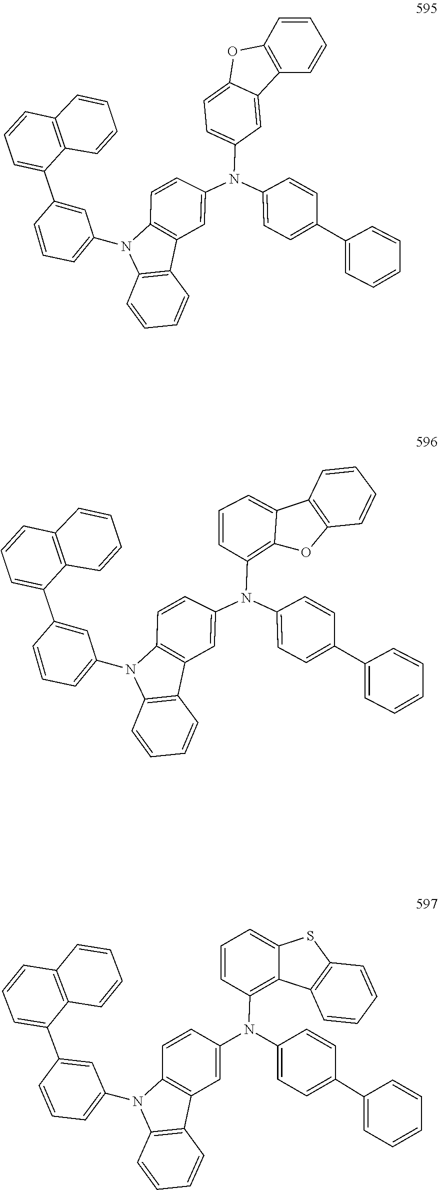 [SMILES: c1ccc(-c2ccc(N(c3ccc4oc5ccccc5c4c3)c3ccc4c(c3)c3ccccc3n4-c3cccc(-c4cccc5ccccc45)c3)cc2)cc1 ; c1ccc(-c2ccc(N(c3ccc4c(c3)c3ccccc3n4-c3cccc(-c4cccc5ccccc45)c3)c3cccc4c3oc3ccccc34)cc2)cc1 ; c1ccc(-c2ccc(N(c3ccc4c(c3)c3ccccc3n4-c3cccc(-c4cccc5ccccc45)c3)c3cccc4sc5ccccc5c34)cc2)cc1] 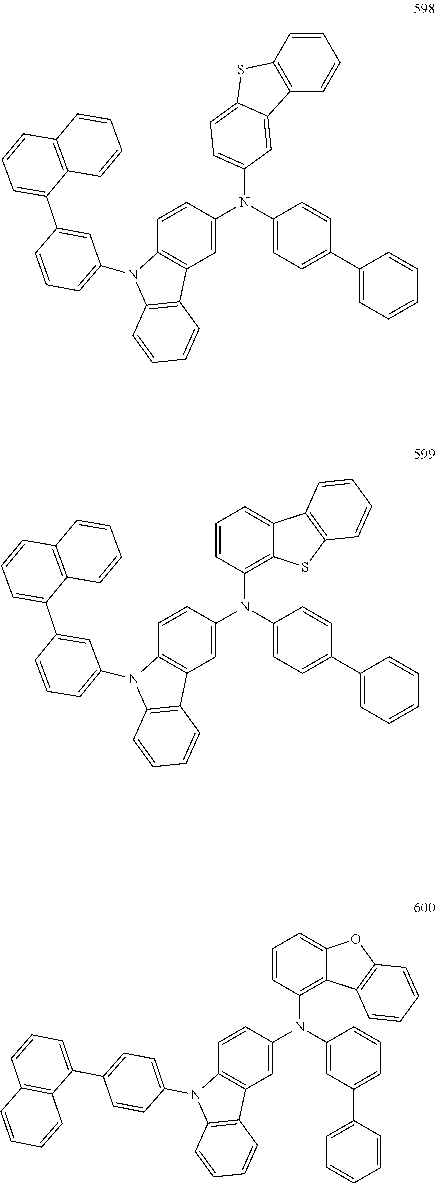 [SMILES: c1ccc(-c2ccc(N(c3ccc4sc5ccccc5c4c3)c3ccc4c(c3)c3ccccc3n4-c3cccc(-c4cccc5ccccc45)c3)cc2)cc1 ; c1ccc(-c2ccc(N(c3ccc4c(c3)c3ccccc3n4-c3cccc(-c4cccc5ccccc45)c3)c3cccc4c3sc3ccccc34)cc2)cc1 ; c1ccc(-c2cccc(N(c3ccc4c(c3)c3ccccc3n4-c3ccc(-c4cccc5ccccc45)cc3)c3cccc4oc5ccccc5c34)c2)cc1] 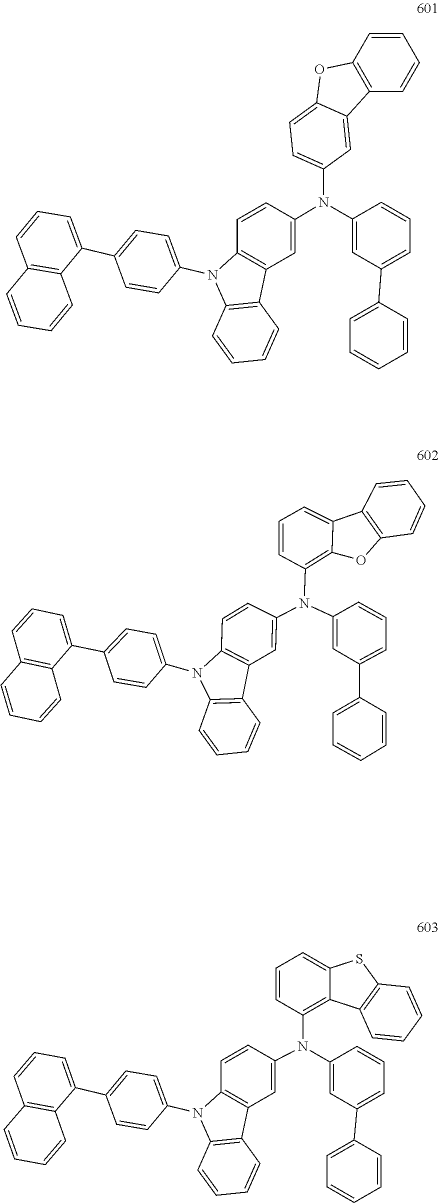 [SMILES: c1ccc(-c2cccc(N(c3ccc4oc5ccccc5c4c3)c3ccc4c(c3)c3ccccc3n4-c3ccc(-c4cccc5ccccc45)cc3)c2)cc1 ; c1ccc(-c2cccc(N(c3ccc4c(c3)c3ccccc3n4-c3ccc(-c4cccc5ccccc45)cc3)c3cccc4c3oc3ccccc34)c2)cc1 ; c1ccc(-c2cccc(N(c3ccc4c(c3)c3ccccc3n4-c3ccc(-c4cccc5ccccc45)cc3)c3cccc4sc5ccccc5c34)c2)cc1] 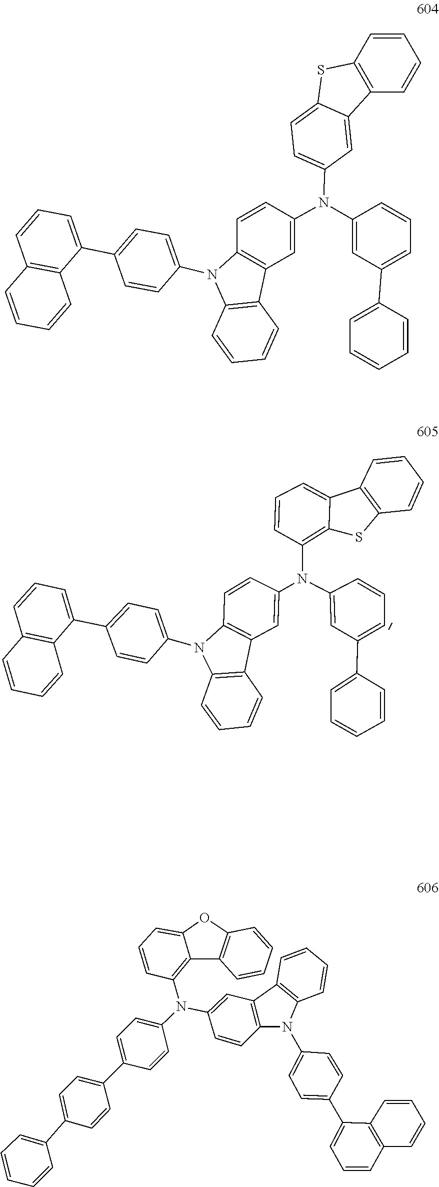 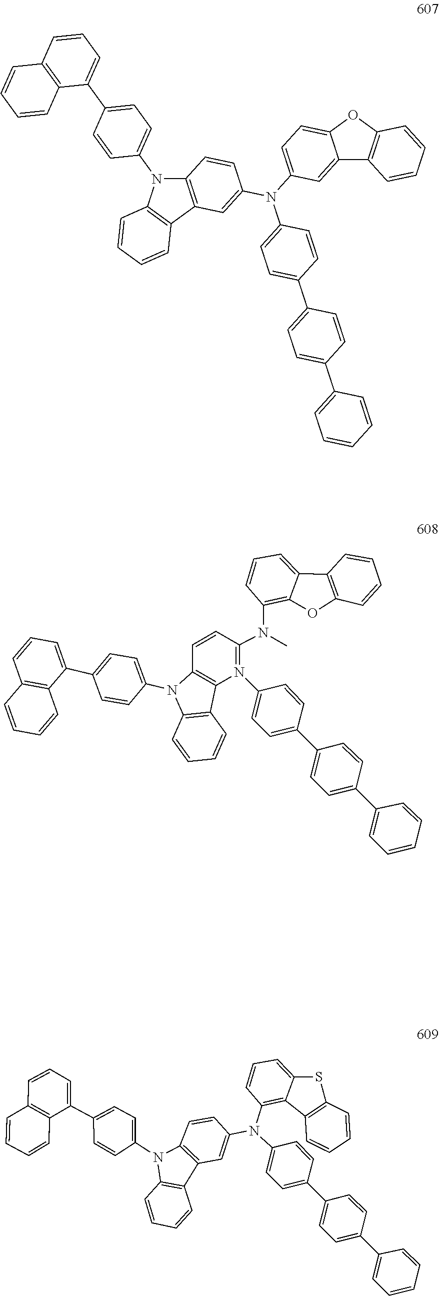 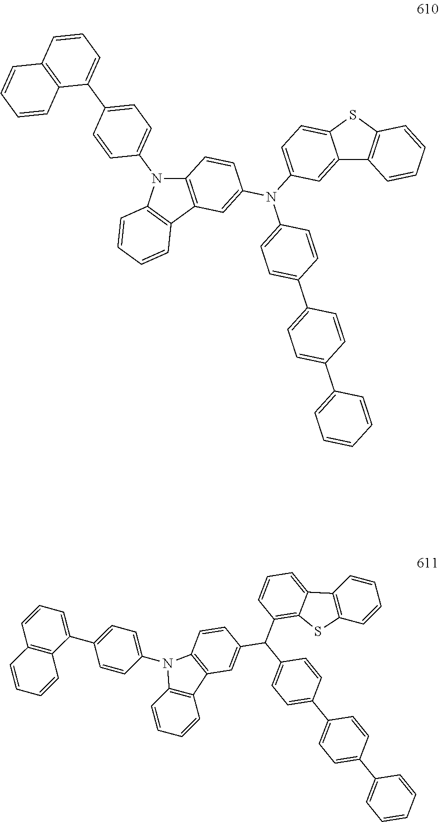 [SMILES: c1ccc(-c2ccc(-c3ccc(N(c4ccc5sc6ccccc6c5c4)c4ccc5c(c4)c4ccccc4n5-c4ccc(-c5cccc6ccccc56)cc4)cc3)cc2)cc1 ; c1ccc(-c2ccc(-c3ccc(C(c4ccc5c(c4)c4ccccc4n5-c4ccc(-c5cccc6ccccc56)cc4)c4cccc5c4sc4ccccc45)cc3)cc2)cc1] 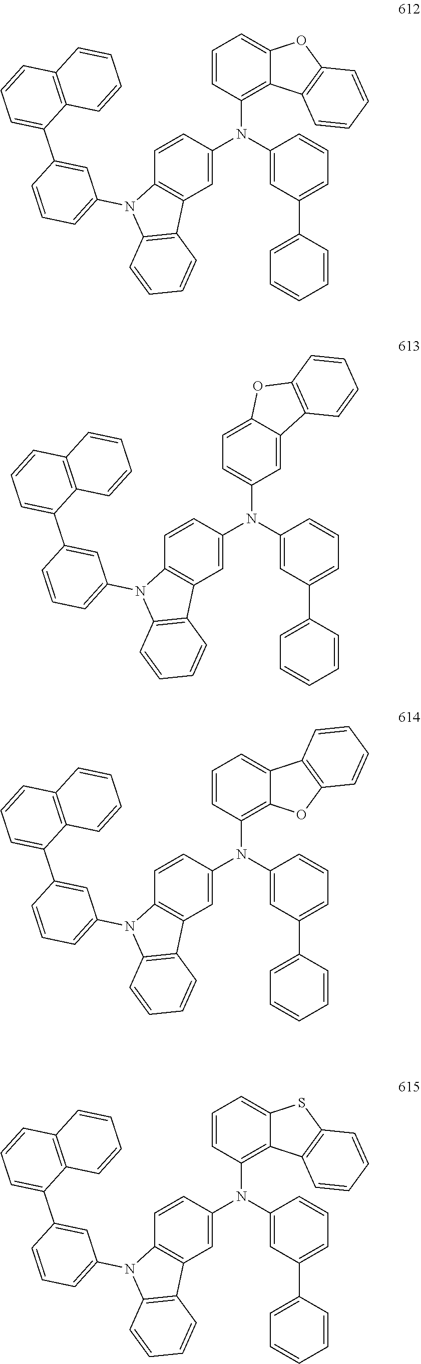 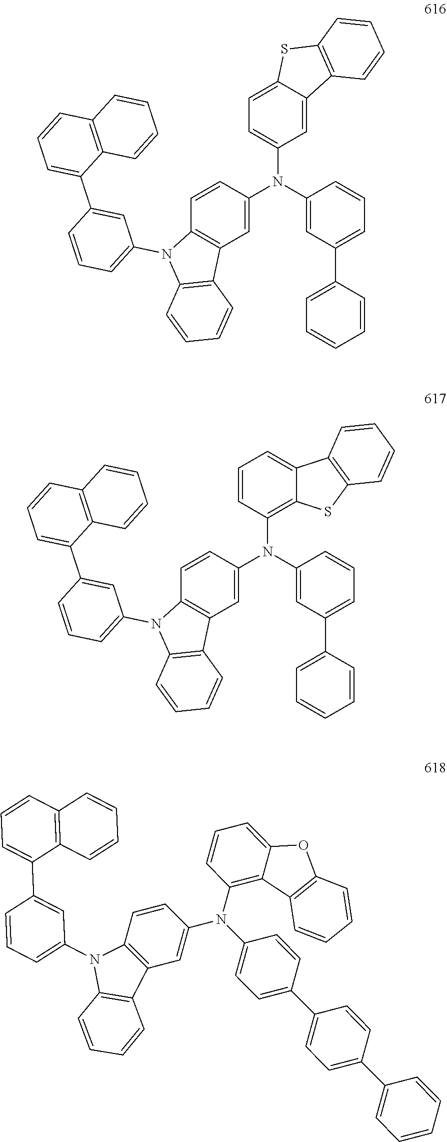 [SMILES: c1ccc(-c2cccc(N(c3ccc4sc5ccccc5c4c3)c3ccc4c(c3)c3ccccc3n4-c3cccc(-c4cccc5ccccc45)c3)c2)cc1 ; c1ccc(-c2cccc(N(c3ccc4c(c3)c3ccccc3n4-c3cccc(-c4cccc5ccccc45)c3)c3cccc4c3sc3ccccc34)c2)cc1 ; c1ccc(-c2ccc(-c3ccc(N(c4ccc5c(c4)c4ccccc4n5-c4cccc(-c5cccc6ccccc56)c4)c4cccc5oc6ccccc6c45)cc3)cc2)cc1] 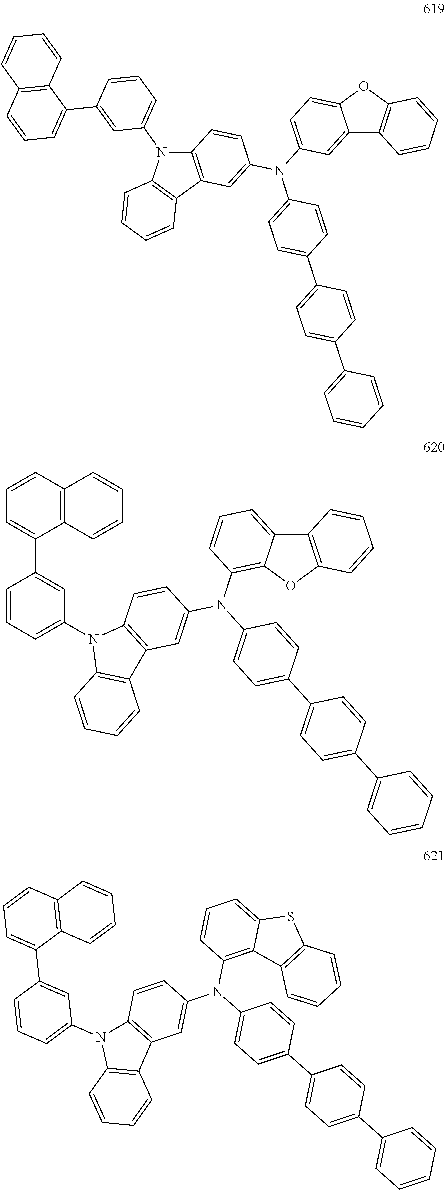 [SMILES: c1ccc(-c2ccc(-c3ccc(N(c4ccc5oc6ccccc6c5c4)c4ccc5c(c4)c4ccccc4n5-c4cccc(-c5cccc6ccccc56)c4)cc3)cc2)cc1 ; c1ccc(-c2ccc(-c3ccc(N(c4ccc5c(c4)c4ccccc4n5-c4cccc(-c5cccc6ccccc56)c4)c4cccc5c4oc4ccccc45)cc3)cc2)cc1 ; c1ccc(-c2ccc(-c3ccc(N(c4ccc5c(c4)c4ccccc4n5-c4cccc(-c5cccc6ccccc56)c4)c4cccc5sc6ccccc6c45)cc3)cc2)cc1] 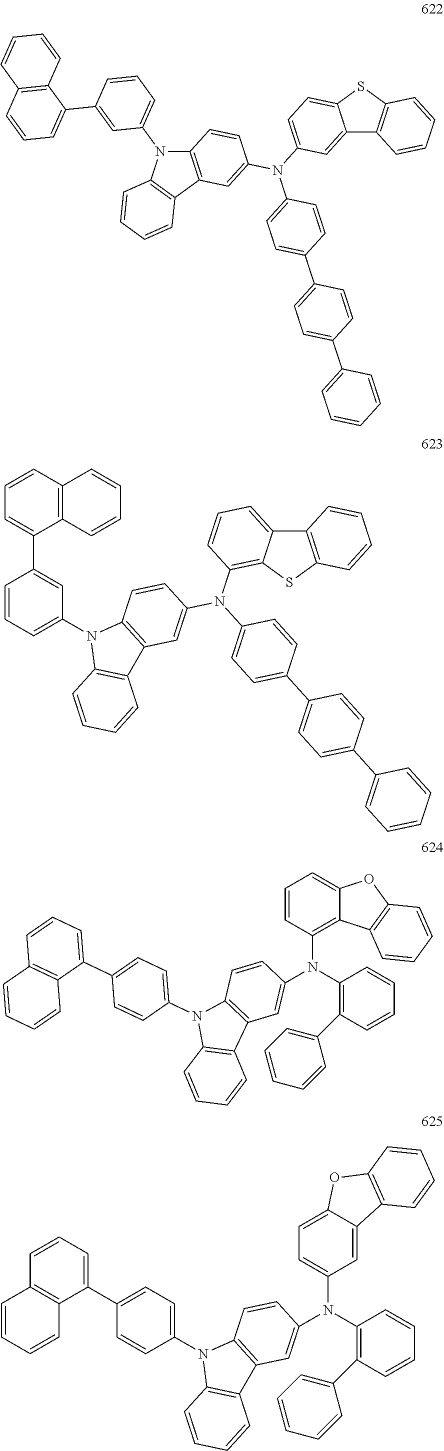 [SMILES: c1ccc(-c2ccc(-c3ccc(N(c4ccc5sc6ccccc6c5c4)c4ccc5c(c4)c4ccccc4n5-c4cccc(-c5cccc6ccccc56)c4)cc3)cc2)cc1 ; c1ccc(-c2ccc(-c3ccc(N(c4ccc5c(c4)c4ccccc4n5-c4cccc(-c5cccc6ccccc56)c4)c4cccc5c4sc4ccccc45)cc3)cc2)cc1 ; c1ccc(-c2ccccc2N(c2ccc3c(c2)c2ccccc2n3-c2ccc(-c3cccc4ccccc34)cc2)c2cccc3oc4ccccc4c23)cc1 ; c1ccc(-c2ccccc2N(c2ccc3oc4ccccc4c3c2)c2ccc3c(c2)c2ccccc2n3-c2ccc(-c3cccc4ccccc34)cc2)cc1] 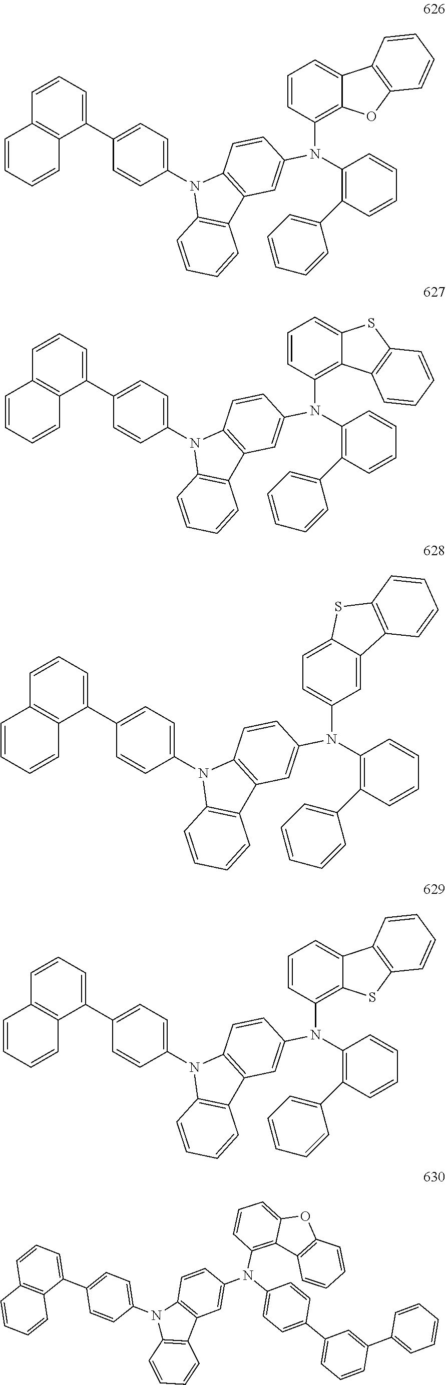 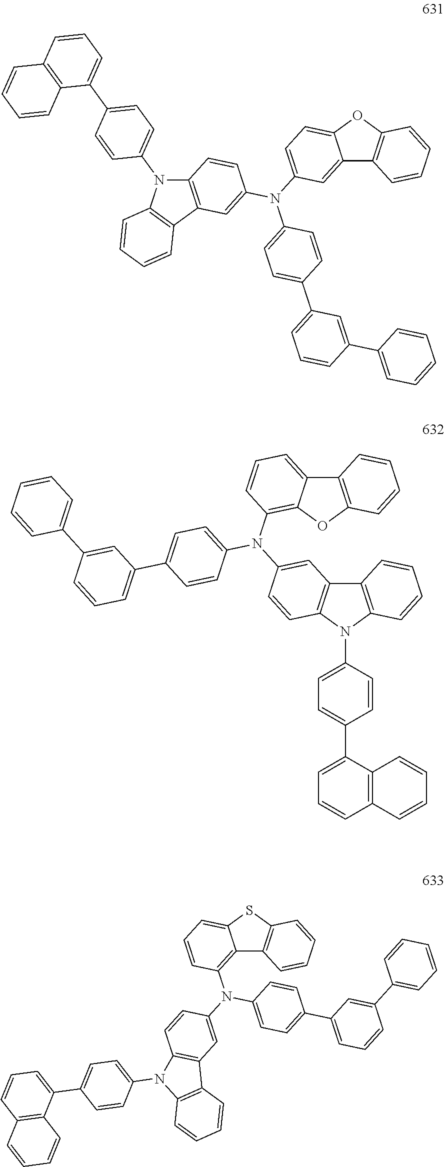 [SMILES: c1ccc(-c2cccc(-c3ccc(N(c4ccc5oc6ccccc6c5c4)c4ccc5c(c4)c4ccccc4n5-c4ccc(-c5cccc6ccccc56)cc4)cc3)c2)cc1 ; c1ccc(-c2cccc(-c3ccc(N(c4ccc5c(c4)c4ccccc4n5-c4ccc(-c5cccc6ccccc56)cc4)c4cccc5c4oc4ccccc45)cc3)c2)cc1 ; c1ccc(-c2cccc(-c3ccc(N(c4ccc5c(c4)c4ccccc4n5-c4ccc(-c5cccc6ccccc56)cc4)c4cccc5sc6ccccc6c45)cc3)c2)cc1] 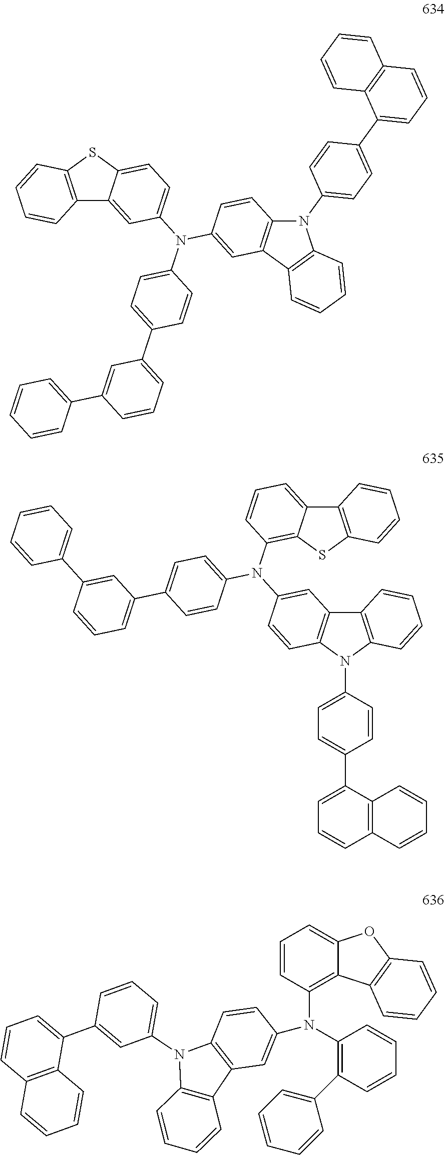 [SMILES: c1ccc(-c2cccc(-c3ccc(N(c4ccc5sc6ccccc6c5c4)c4ccc5c(c4)c4ccccc4n5-c4ccc(-c5cccc6ccccc56)cc4)cc3)c2)cc1 ; c1ccc(-c2cccc(-c3ccc(N(c4ccc5c(c4)c4ccccc4n5-c4ccc(-c5cccc6ccccc56)cc4)c4cccc5c4sc4ccccc45)cc3)c2)cc1 ; c1ccc(-c2ccccc2N(c2ccc3c(c2)c2ccccc2n3-c2cccc(-c3cccc4ccccc34)c2)c2cccc3oc4ccccc4c23)cc1] 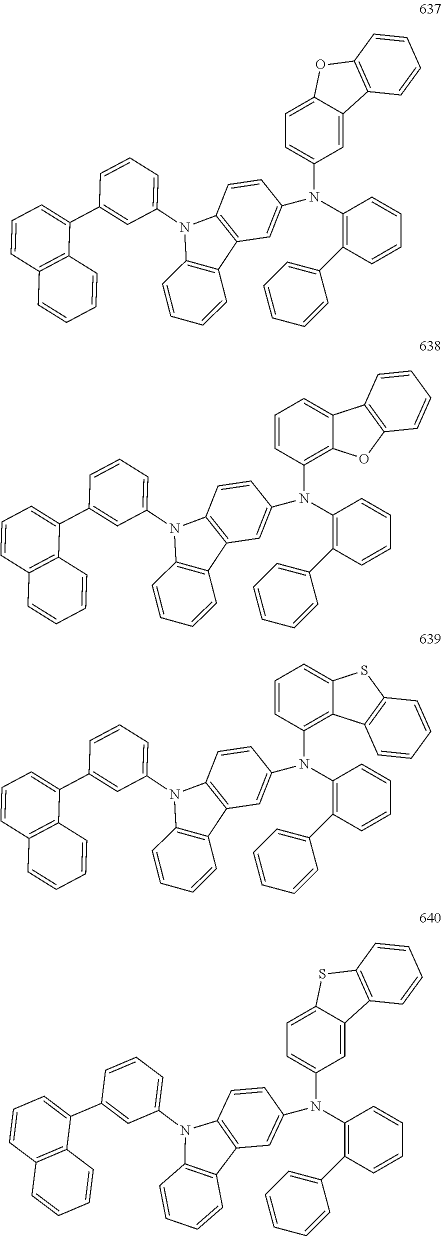 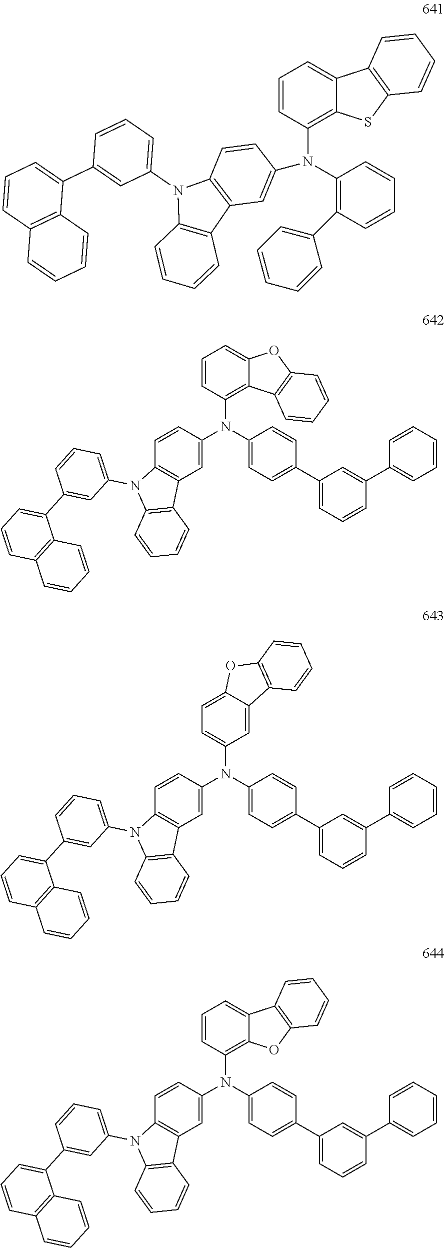 [SMILES: c1ccc(-c2ccccc2N(c2ccc3c(c2)c2ccccc2n3-c2cccc(-c3cccc4ccccc34)c2)c2cccc3c2sc2ccccc23)cc1 ; c1ccc(-c2cccc(-c3ccc(N(c4ccc5c(c4)c4ccccc4n5-c4cccc(-c5cccc6ccccc56)c4)c4cccc5oc6ccccc6c45)cc3)c2)cc1 ; c1ccc(-c2cccc(-c3ccc(N(c4ccc5oc6ccccc6c5c4)c4ccc5c(c4)c4ccccc4n5-c4cccc(-c5cccc6ccccc56)c4)cc3)c2)cc1 ; c1ccc(-c2cccc(-c3ccc(N(c4ccc5c(c4)c4ccccc4n5-c4cccc(-c5cccc6ccccc56)c4)c4cccc5c4oc4ccccc45)cc3)c2)cc1] 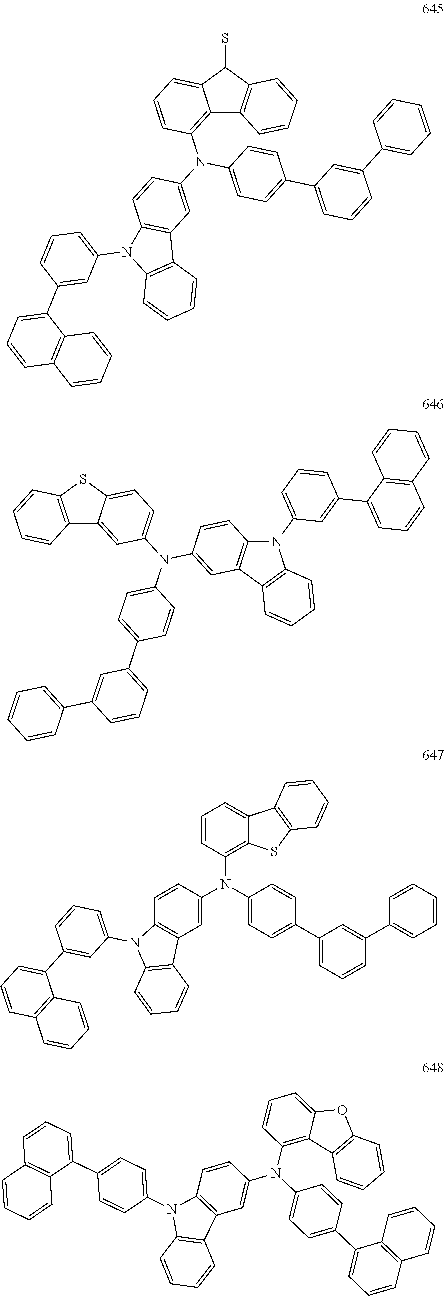 [SMILES: SC1c2ccccc2-c2c1cccc2N(c1ccc(-c2cccc(-c3ccccc3)c2)cc1)c1ccc2c(c1)c1ccccc1n2-c1cccc(-c2cccc3ccccc23)c1 ; c1ccc(-c2cccc(-c3ccc(N(c4ccc5sc6ccccc6c5c4)c4ccc5c(c4)c4ccccc4n5-c4cccc(-c5cccc6ccccc56)c4)cc3)c2)cc1 ; c1ccc(-c2cccc(-c3ccc(N(c4ccc5c(c4)c4ccccc4n5-c4cccc(-c5cccc6ccccc56)c4)c4cccc5c4sc4ccccc45)cc3)c2)cc1 ; c1ccc2c(-c3ccc(N(c4ccc5c(c4)c4ccccc4n5-c4ccc(-c5cccc6ccccc56)cc4)c4cccc5oc6ccccc6c45)cc3)cccc2c1] 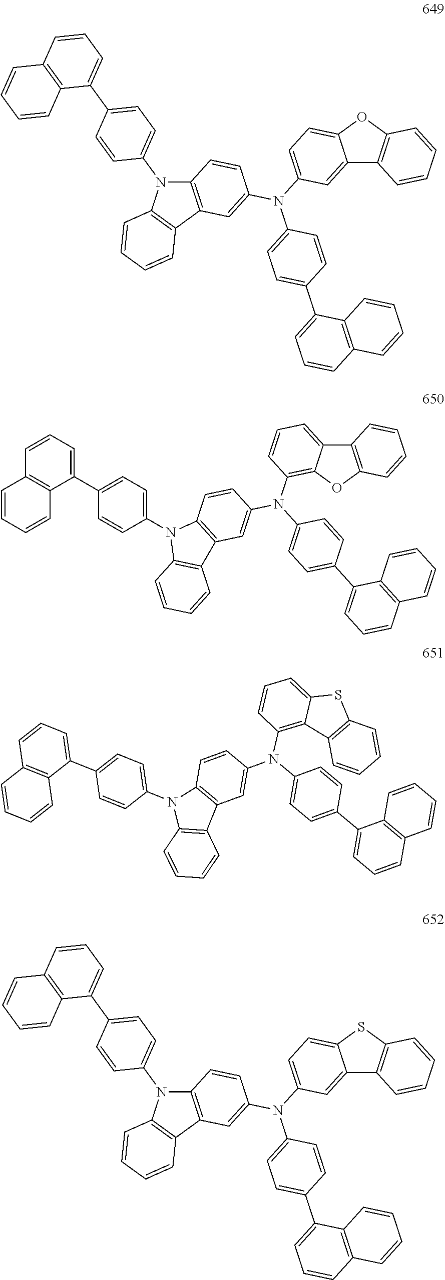 [SMILES: c1ccc2c(-c3ccc(N(c4ccc5oc6ccccc6c5c4)c4ccc5c(c4)c4ccccc4n5-c4ccc(-c5cccc6ccccc56)cc4)cc3)cccc2c1 ; c1ccc2c(-c3ccc(N(c4ccc5c(c4)c4ccccc4n5-c4ccc(-c5cccc6ccccc56)cc4)c4cccc5c4oc4ccccc45)cc3)cccc2c1 ; c1ccc2c(-c3ccc(N(c4ccc5c(c4)c4ccccc4n5-c4ccc(-c5cccc6ccccc56)cc4)c4cccc5sc6ccccc6c45)cc3)cccc2c1 ; c1ccc2c(-c3ccc(N(c4ccc5sc6ccccc6c5c4)c4ccc5c(c4)c4ccccc4n5-c4ccc(-c5cccc6ccccc56)cc4)cc3)cccc2c1] 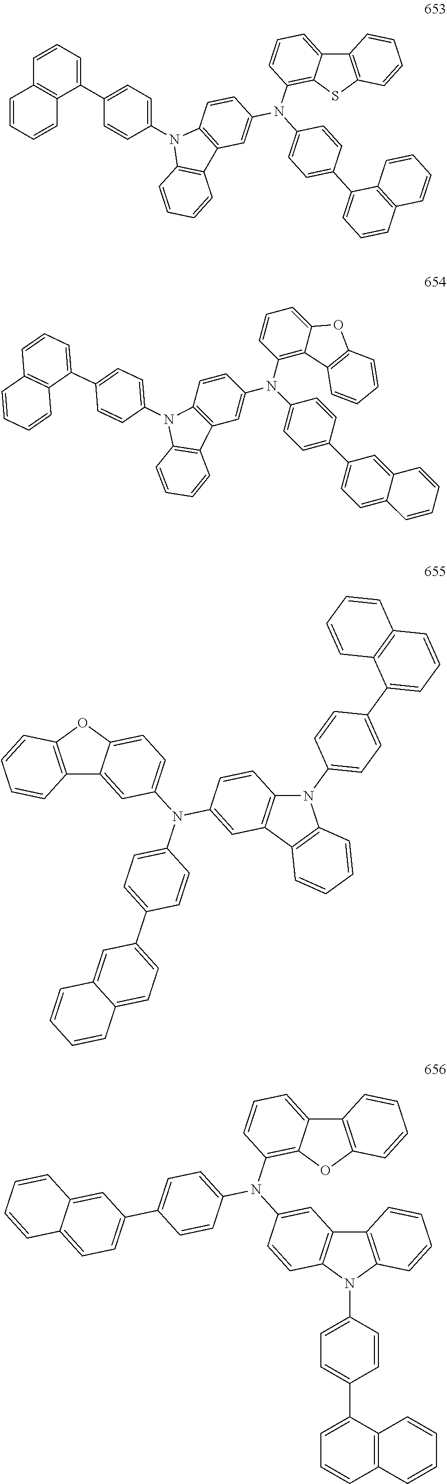 [SMILES: c1ccc2c(-c3ccc(N(c4ccc5c(c4)c4ccccc4n5-c4ccc(-c5cccc6ccccc56)cc4)c4cccc5c4sc4ccccc45)cc3)cccc2c1 ; c1ccc2cc(-c3ccc(N(c4ccc5c(c4)c4ccccc4n5-c4ccc(-c5cccc6ccccc56)cc4)c4cccc5oc6ccccc6c45)cc3)ccc2c1 ; c1ccc2cc(-c3ccc(N(c4ccc5oc6ccccc6c5c4)c4ccc5c(c4)c4ccccc4n5-c4ccc(-c5cccc6ccccc56)cc4)cc3)ccc2c1 ; c1ccc2cc(-c3ccc(N(c4ccc5c(c4)c4ccccc4n5-c4ccc(-c5cccc6ccccc56)cc4)c4cccc5c4oc4ccccc45)cc3)ccc2c1] 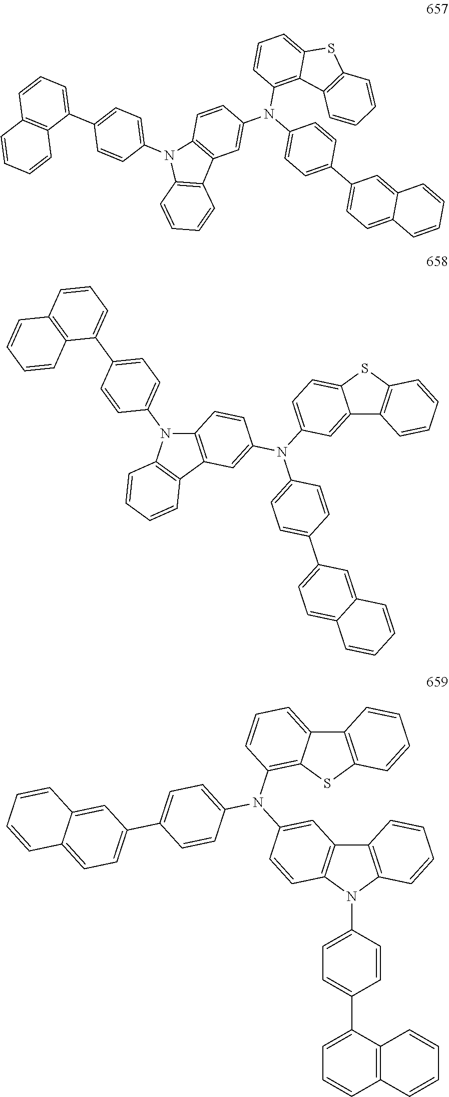 [SMILES: c1ccc2cc(-c3ccc(N(c4ccc5c(c4)c4ccccc4n5-c4ccc(-c5cccc6ccccc56)cc4)c4cccc5sc6ccccc6c45)cc3)ccc2c1 ; c1ccc2cc(-c3ccc(N(c4ccc5sc6ccccc6c5c4)c4ccc5c(c4)c4ccccc4n5-c4ccc(-c5cccc6ccccc56)cc4)cc3)ccc2c1 ; c1ccc2cc(-c3ccc(N(c4ccc5c(c4)c4ccccc4n5-c4ccc(-c5cccc6ccccc56)cc4)c4cccc5c4sc4ccccc45)cc3)ccc2c1] 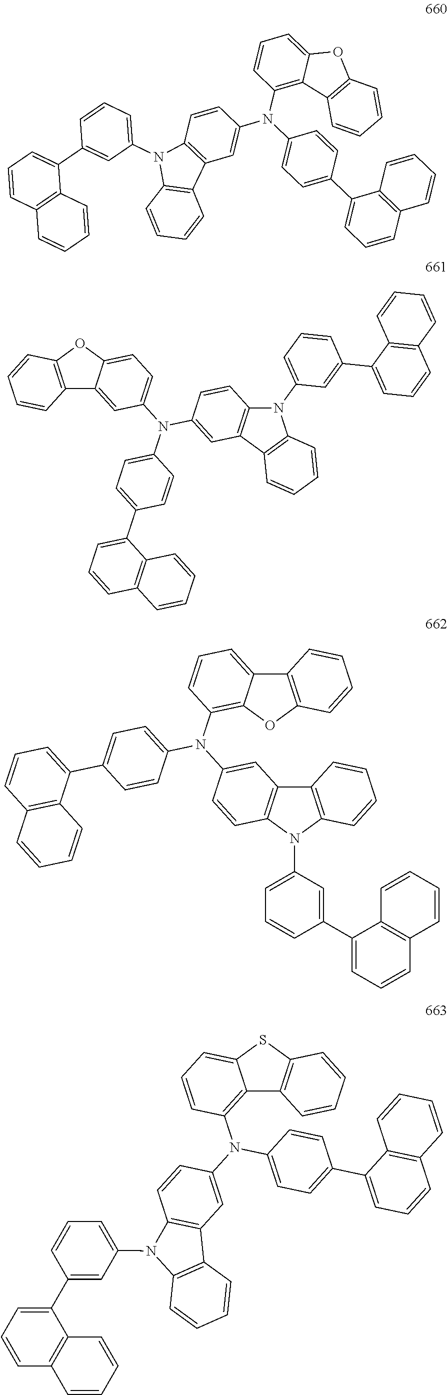 [SMILES: c1cc(-c2cccc3ccccc23)cc(-n2c3ccccc3c3cc(N(c4ccc(-c5cccc6ccccc56)cc4)c4cccc5oc6ccccc6c45)ccc32)c1 ; c1cc(-c2cccc3ccccc23)cc(-n2c3ccccc3c3cc(N(c4ccc(-c5cccc6ccccc56)cc4)c4ccc5oc6ccccc6c5c4)ccc32)c1 ; c1cc(-c2cccc3ccccc23)cc(-n2c3ccccc3c3cc(N(c4ccc(-c5cccc6ccccc56)cc4)c4cccc5c4oc4ccccc45)ccc32)c1 ; c1cc(-c2cccc3ccccc23)cc(-n2c3ccccc3c3cc(N(c4ccc(-c5cccc6ccccc56)cc4)c4cccc5sc6ccccc6c45)ccc32)c1] 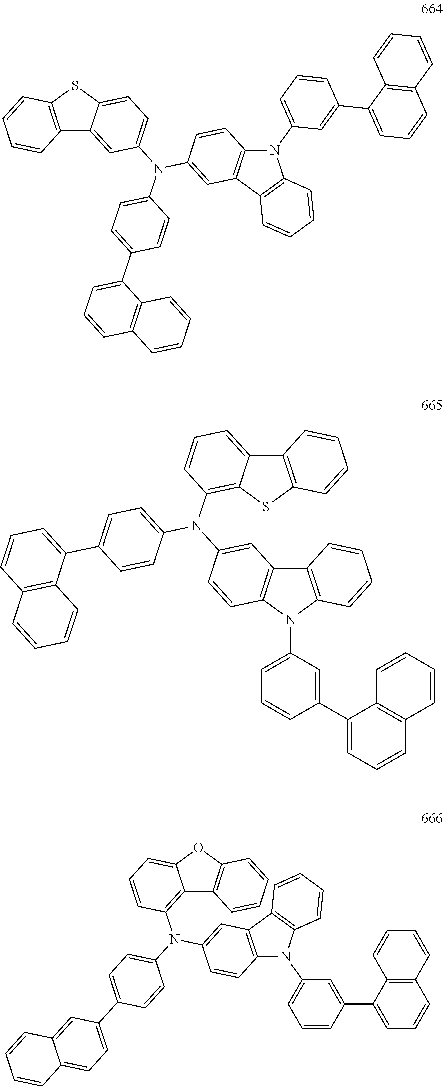 [SMILES: c1cc(-c2cccc3ccccc23)cc(-n2c3ccccc3c3cc(N(c4ccc(-c5cccc6ccccc56)cc4)c4ccc5sc6ccccc6c5c4)ccc32)c1 ; c1cc(-c2cccc3ccccc23)cc(-n2c3ccccc3c3cc(N(c4ccc(-c5cccc6ccccc56)cc4)c4cccc5c4sc4ccccc45)ccc32)c1 ; c1cc(-c2cccc3ccccc23)cc(-n2c3ccccc3c3cc(N(c4ccc(-c5ccc6ccccc6c5)cc4)c4cccc5oc6ccccc6c45)ccc32)c1] 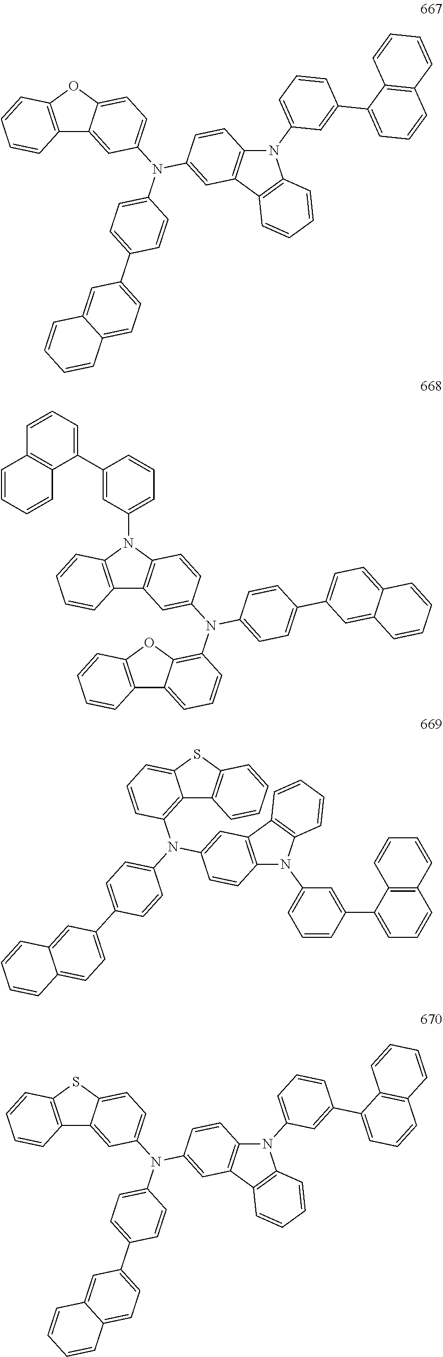 [SMILES: c1cc(-c2cccc3ccccc23)cc(-n2c3ccccc3c3cc(N(c4ccc(-c5ccc6ccccc6c5)cc4)c4ccc5oc6ccccc6c5c4)ccc32)c1 ; c1cc(-c2cccc3ccccc23)cc(-n2c3ccccc3c3cc(N(c4ccc(-c5ccc6ccccc6c5)cc4)c4cccc5c4oc4ccccc45)ccc32)c1 ; c1cc(-c2cccc3ccccc23)cc(-n2c3ccccc3c3cc(N(c4ccc(-c5ccc6ccccc6c5)cc4)c4cccc5sc6ccccc6c45)ccc32)c1 ; c1cc(-c2cccc3ccccc23)cc(-n2c3ccccc3c3cc(N(c4ccc(-c5ccc6ccccc6c5)cc4)c4ccc5sc6ccccc6c5c4)ccc32)c1] 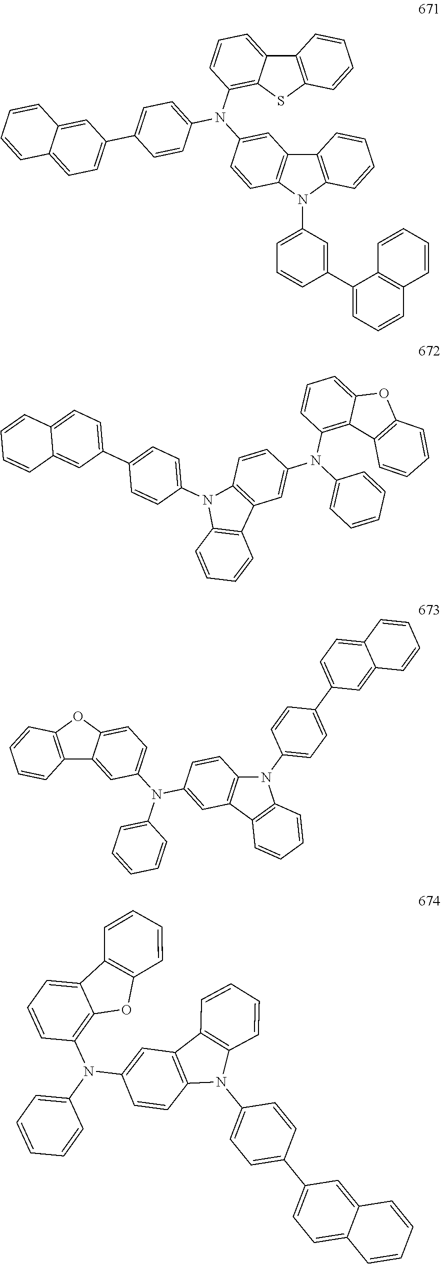 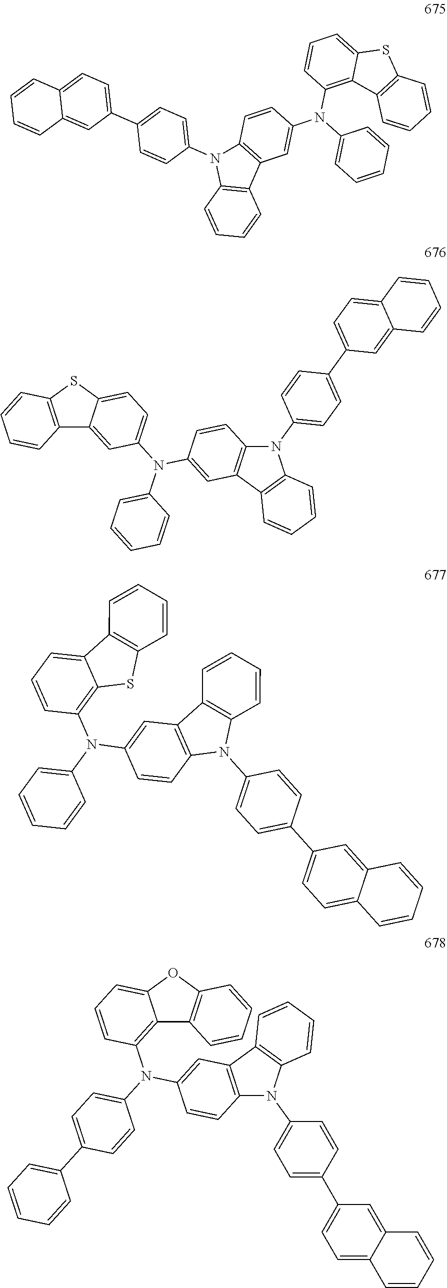 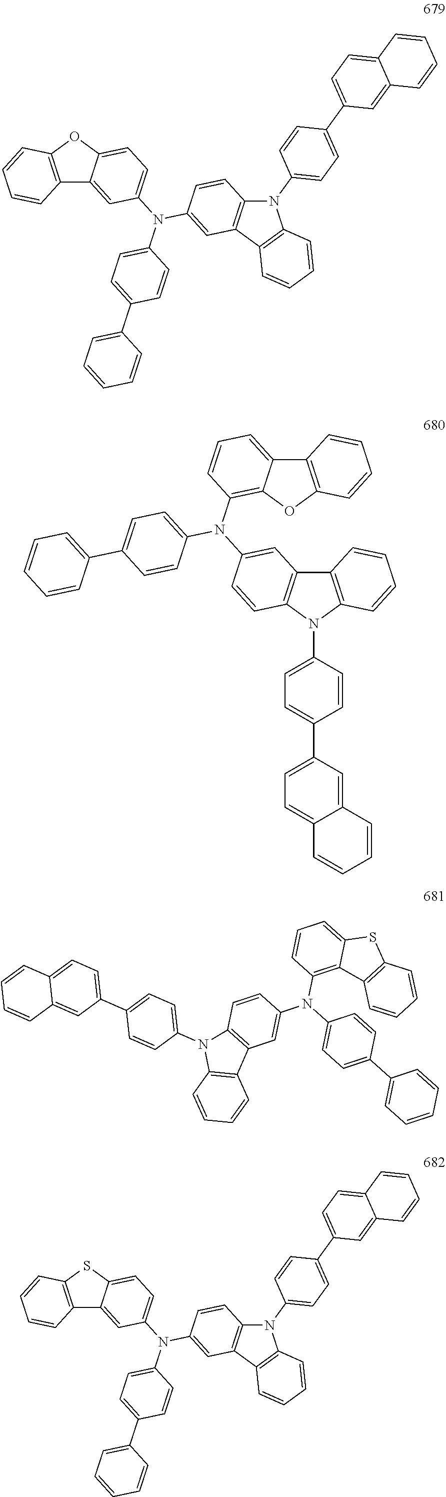 [SMILES: c1ccc(-c2ccc(N(c3ccc4oc5ccccc5c4c3)c3ccc4c(c3)c3ccccc3n4-c3ccc(-c4ccc5ccccc5c4)cc3)cc2)cc1 ; c1ccc(-c2ccc(N(c3ccc4c(c3)c3ccccc3n4-c3ccc(-c4ccc5ccccc5c4)cc3)c3cccc4c3oc3ccccc34)cc2)cc1 ; c1ccc(-c2ccc(N(c3ccc4c(c3)c3ccccc3n4-c3ccc(-c4ccc5ccccc5c4)cc3)c3cccc4sc5ccccc5c34)cc2)cc1 ; c1ccc(-c2ccc(N(c3ccc4sc5ccccc5c4c3)c3ccc4c(c3)c3ccccc3n4-c3ccc(-c4ccc5ccccc5c4)cc3)cc2)cc1] 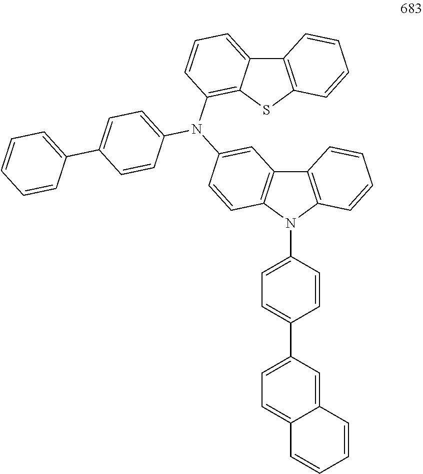 [SMILES: c1ccc(-c2ccc(N(c3ccc4c(c3)c3ccccc3n4-c3ccc(-c4ccc5ccccc5c4)cc3)c3cccc4c3sc3ccccc34)cc2)cc1] 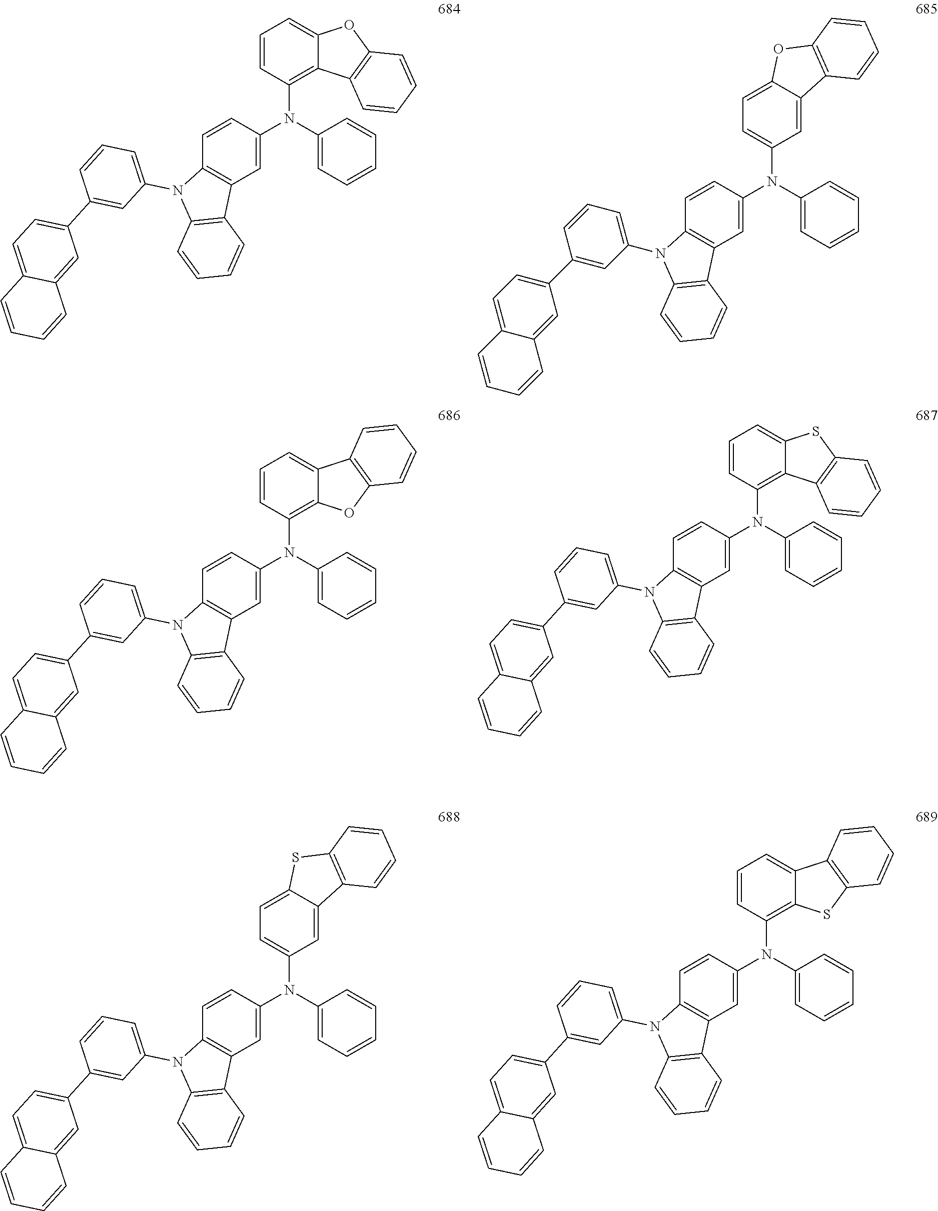 [SMILES: c1ccc(N(c2ccc3c(c2)c2ccccc2n3-c2cccc(-c3ccc4ccccc4c3)c2)c2cccc3oc4ccccc4c23)cc1 ; c1ccc(N(c2ccc3oc4ccccc4c3c2)c2ccc3c(c2)c2ccccc2n3-c2cccc(-c3ccc4ccccc4c3)c2)cc1 ; c1ccc(N(c2ccc3c(c2)c2ccccc2n3-c2cccc(-c3ccc4ccccc4c3)c2)c2cccc3c2oc2ccccc23)cc1 ; c1ccc(N(c2ccc3c(c2)c2ccccc2n3-c2cccc(-c3ccc4ccccc4c3)c2)c2cccc3sc4ccccc4c23)cc1 ; c1ccc(N(c2ccc3sc4ccccc4c3c2)c2ccc3c(c2)c2ccccc2n3-c2cccc(-c3ccc4ccccc4c3)c2)cc1 ; c1ccc(N(c2ccc3c(c2)c2ccccc2n3-c2cccc(-c3ccc4ccccc4c3)c2)c2cccc3c2sc2ccccc23)cc1] 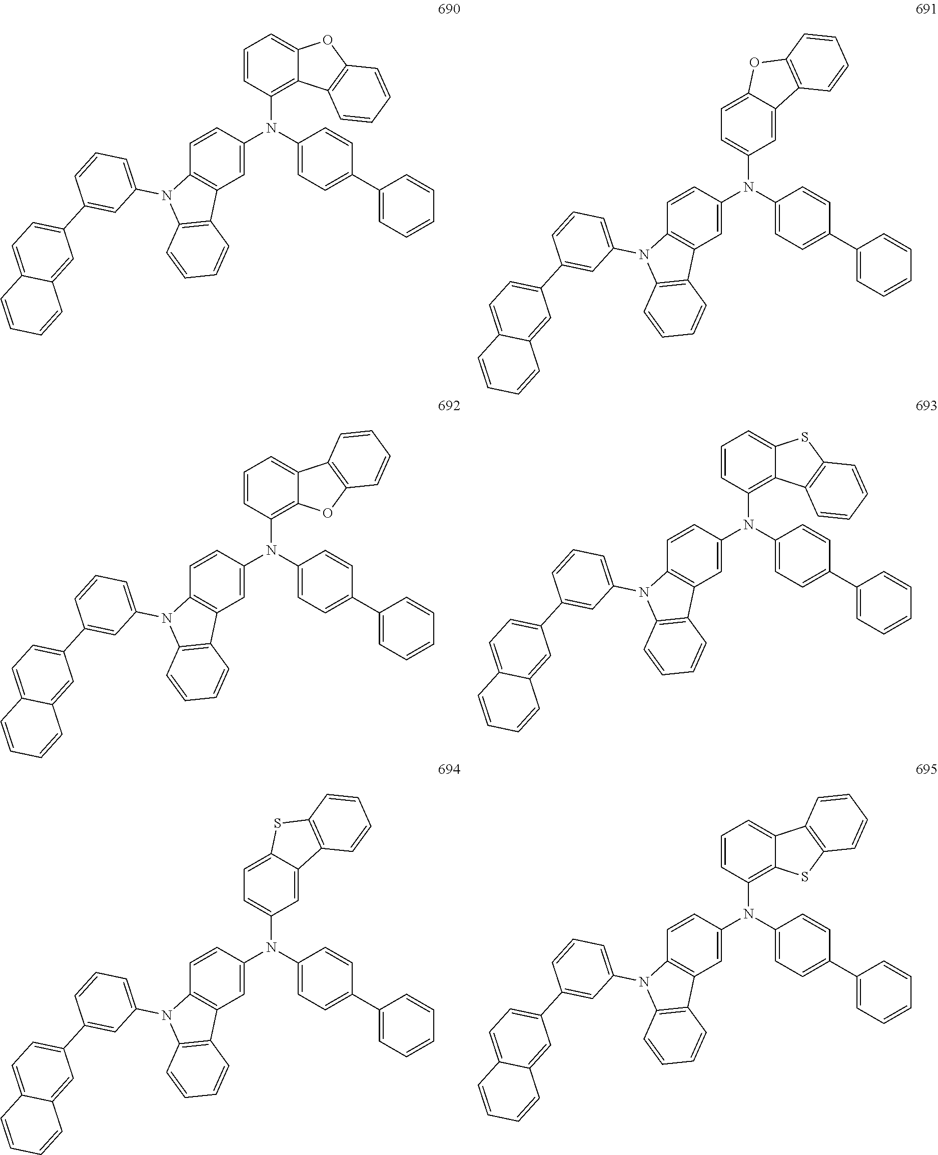 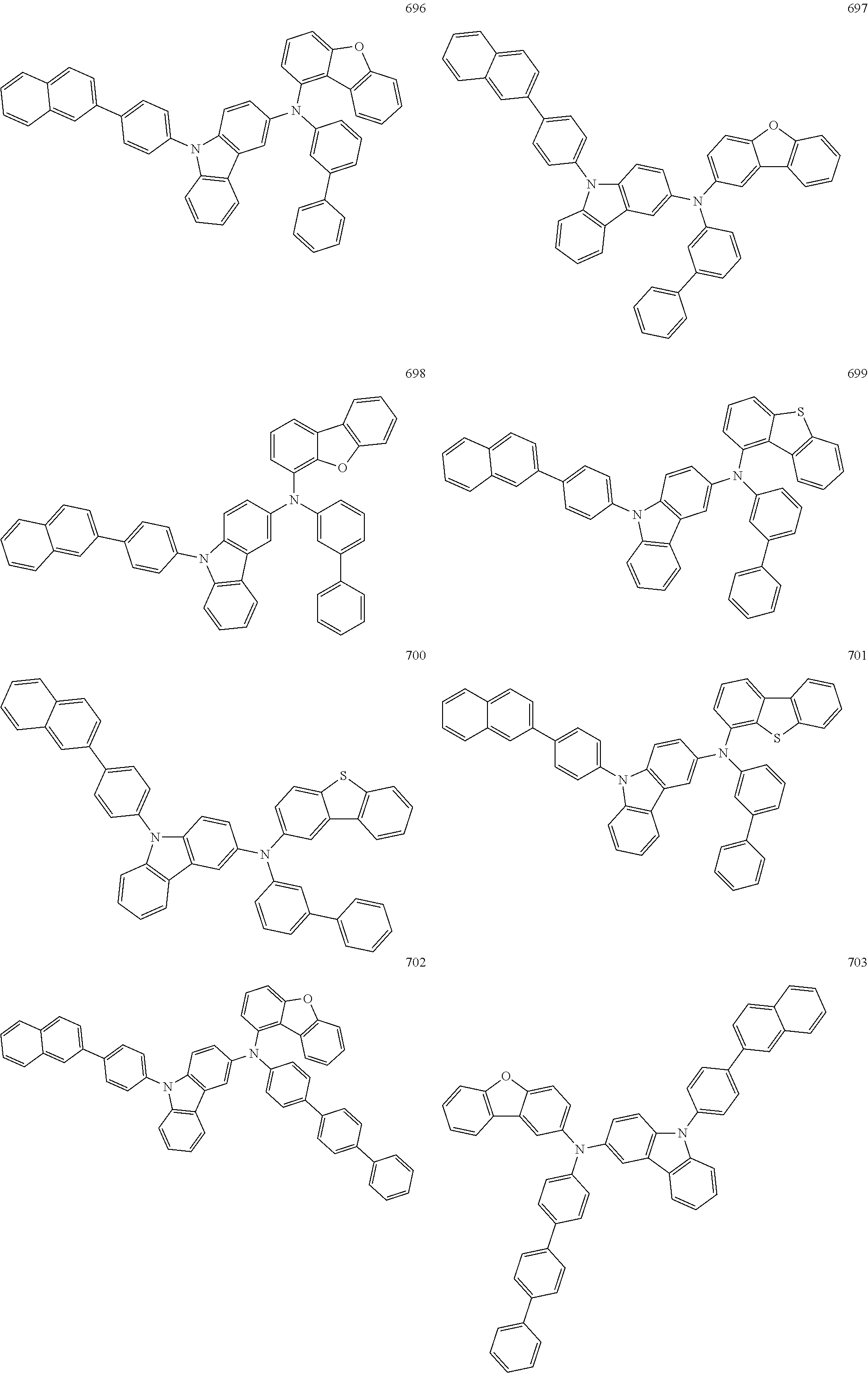 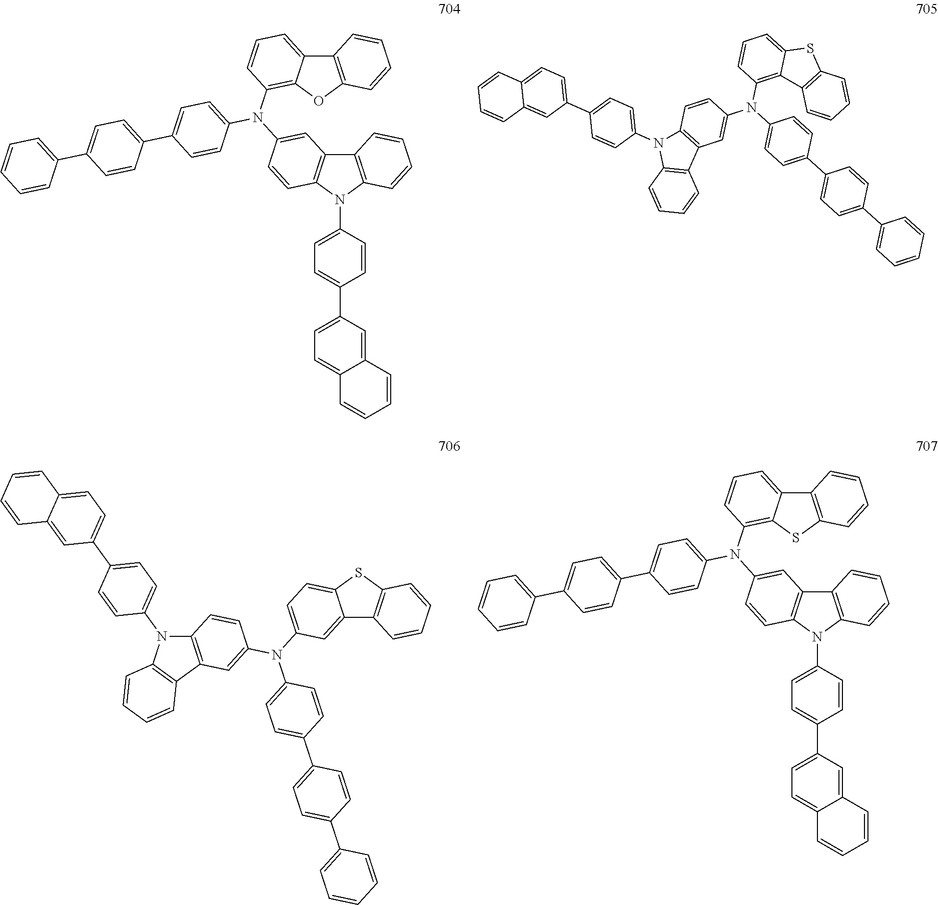 [SMILES: c1ccc(-c2ccc(-c3ccc(N(c4ccc5c(c4)c4ccccc4n5-c4ccc(-c5ccc6ccccc6c5)cc4)c4cccc5c4oc4ccccc45)cc3)cc2)cc1 ; c1ccc(-c2ccc(-c3ccc(N(c4ccc5c(c4)c4ccccc4n5-c4ccc(-c5ccc6ccccc6c5)cc4)c4cccc5sc6ccccc6c45)cc3)cc2)cc1 ; c1ccc(-c2ccc(-c3ccc(N(c4ccc5sc6ccccc6c5c4)c4ccc5c(c4)c4ccccc4n5-c4ccc(-c5ccc6ccccc6c5)cc4)cc3)cc2)cc1 ; c1ccc(-c2ccc(-c3ccc(N(c4ccc5c(c4)c4ccccc4n5-c4ccc(-c5ccc6ccccc6c5)cc4)c4cccc5c4sc4ccccc45)cc3)cc2)cc1] 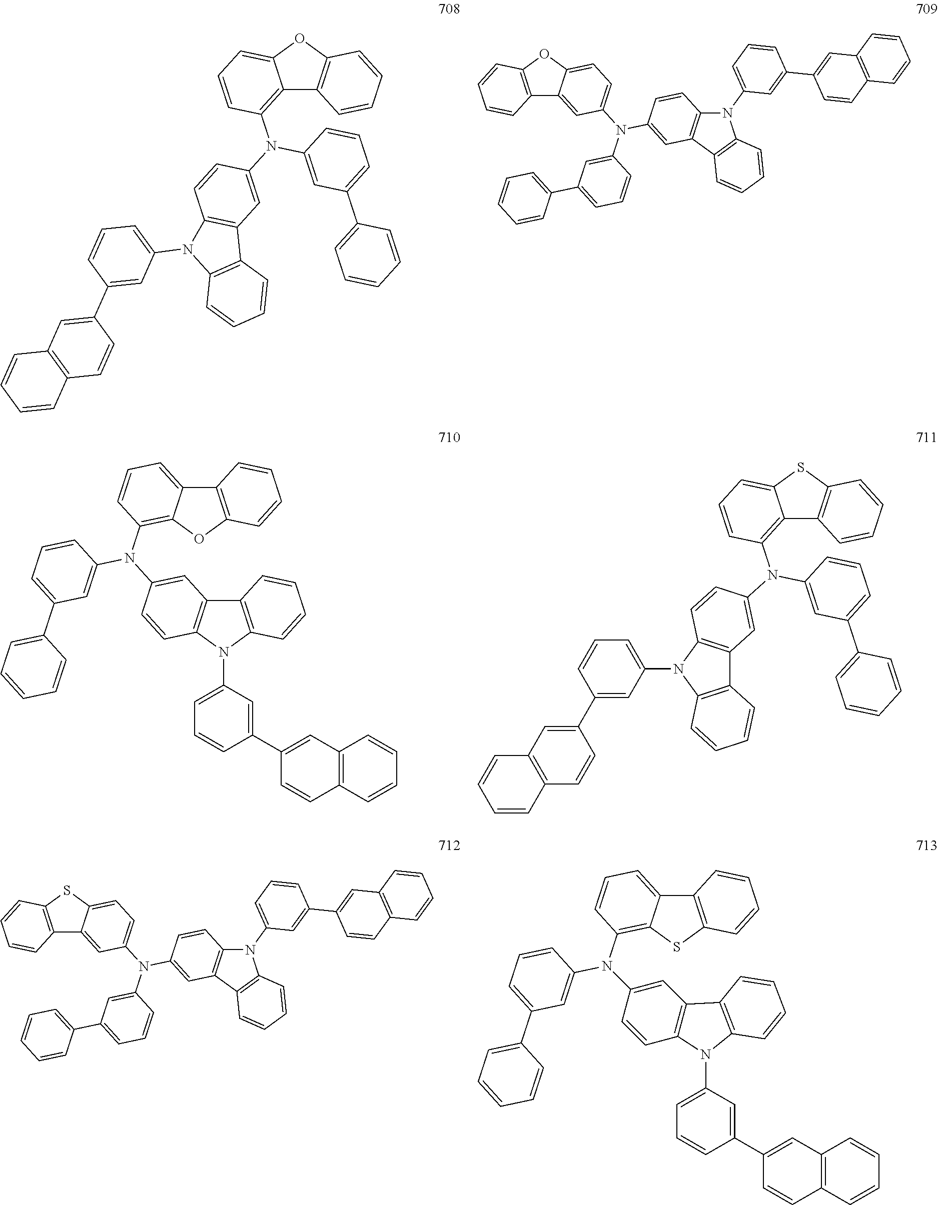 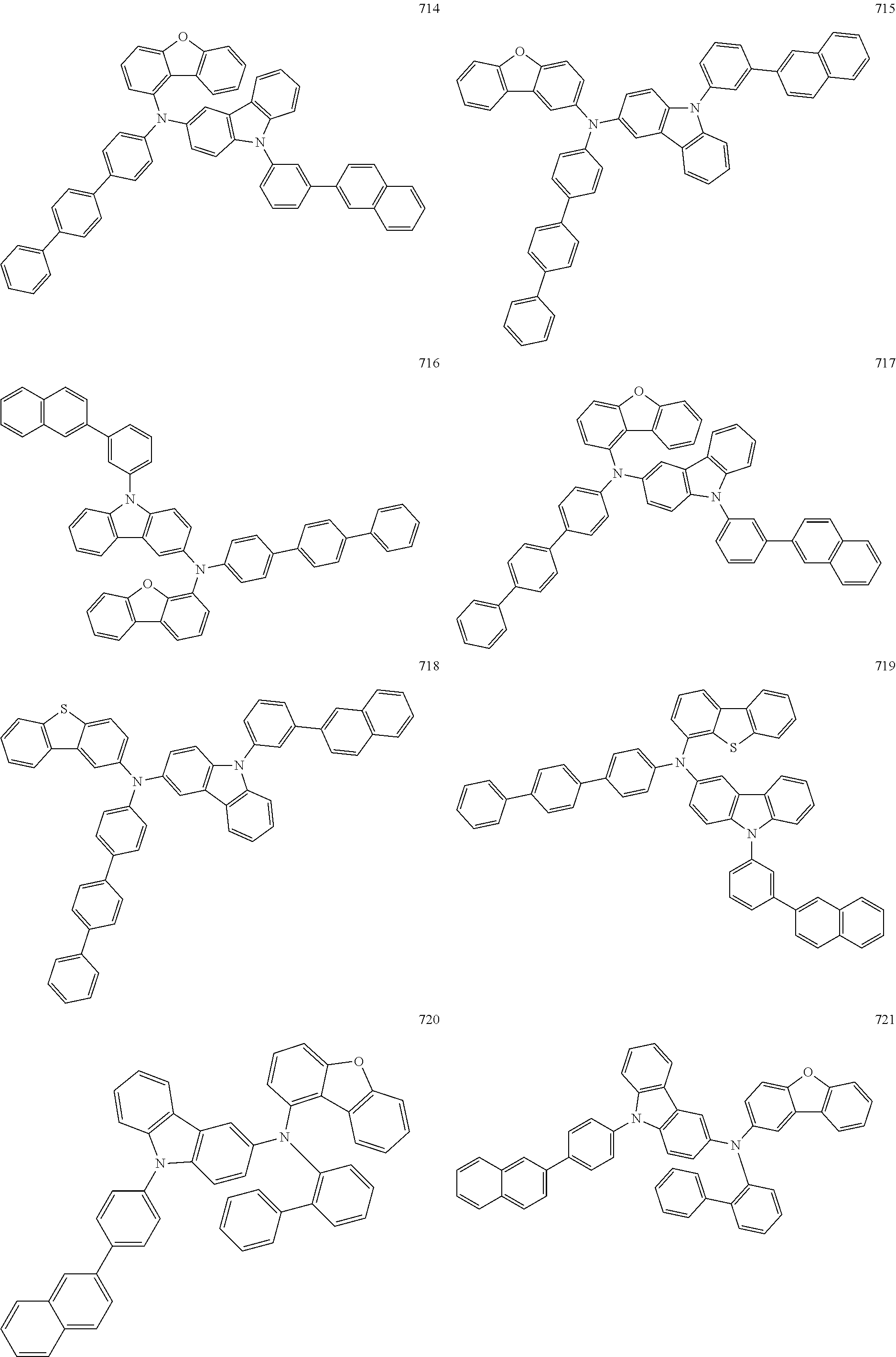 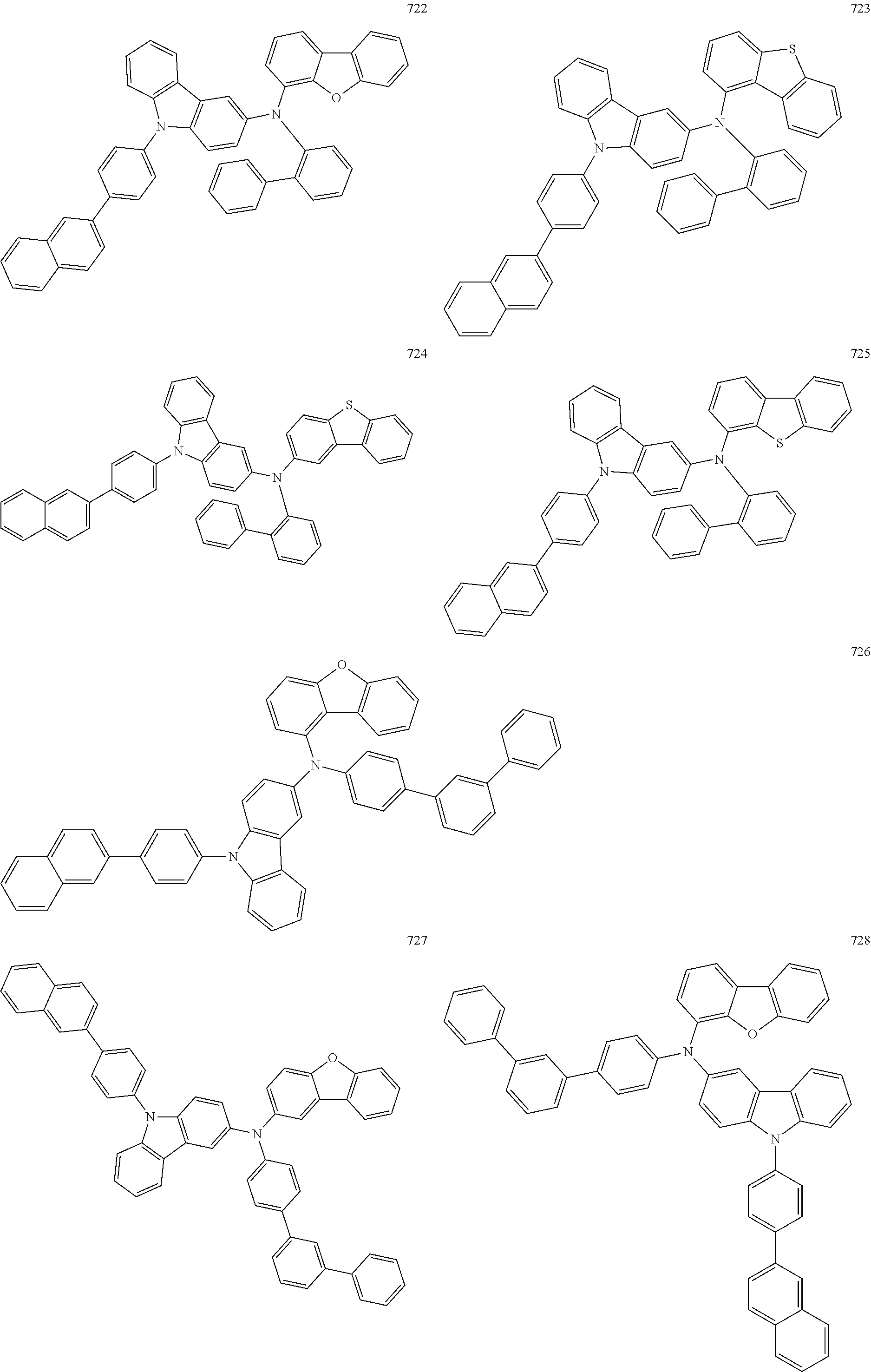 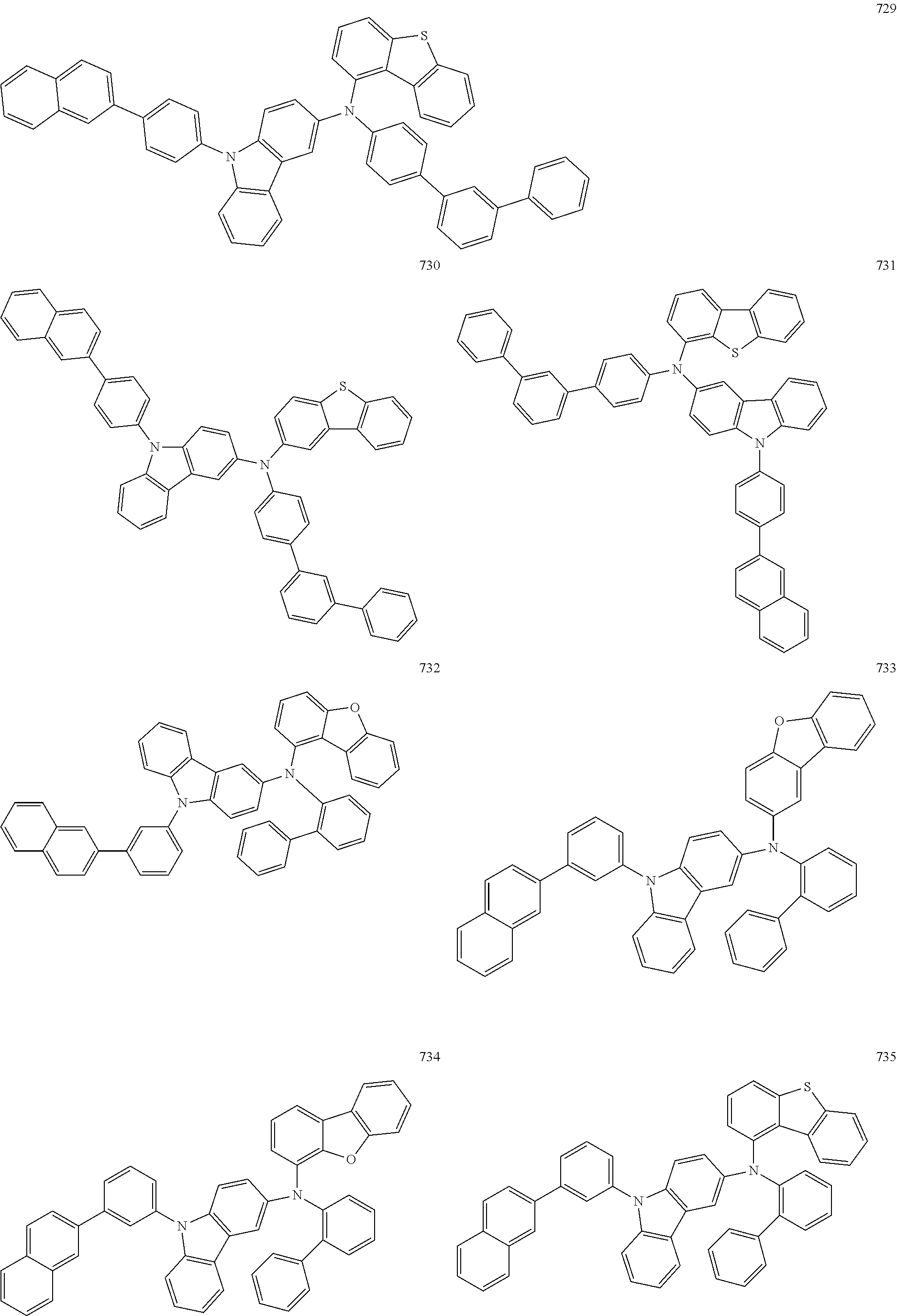 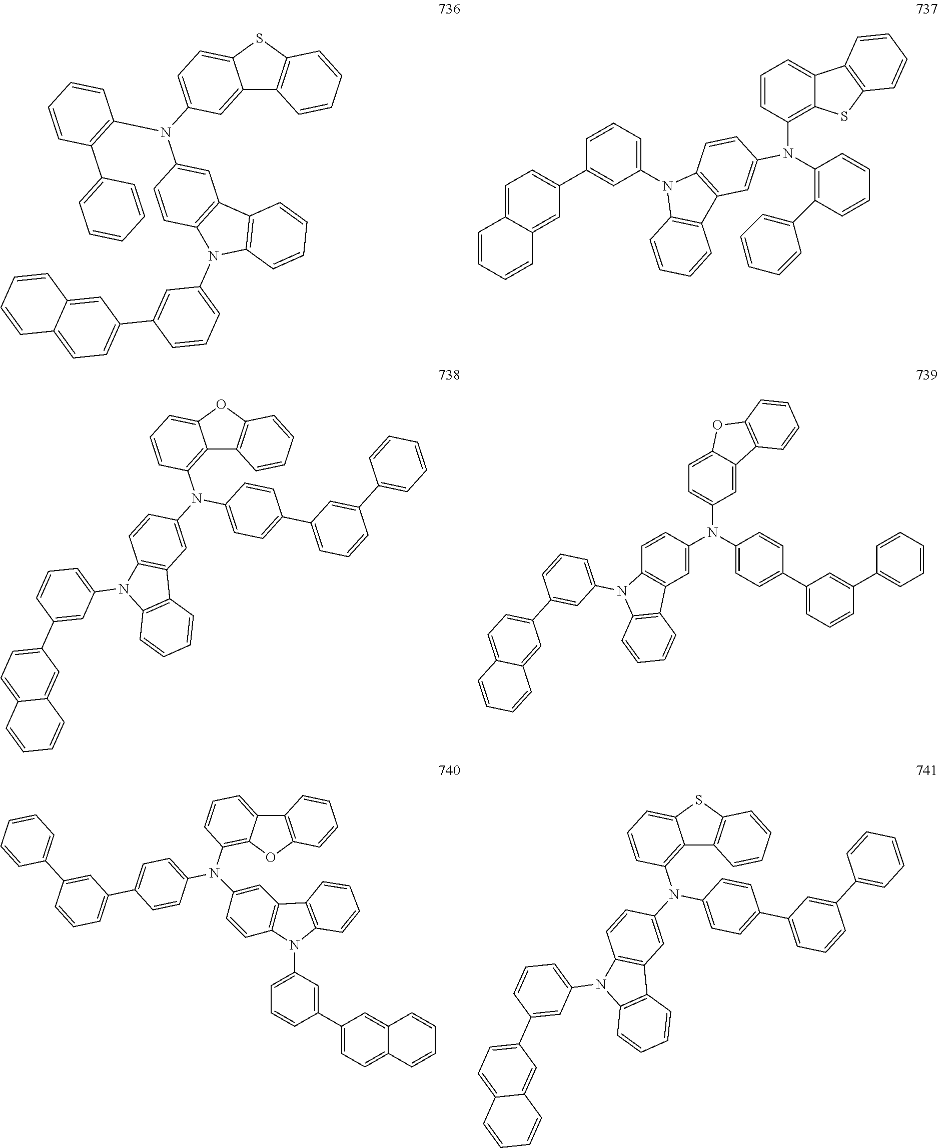 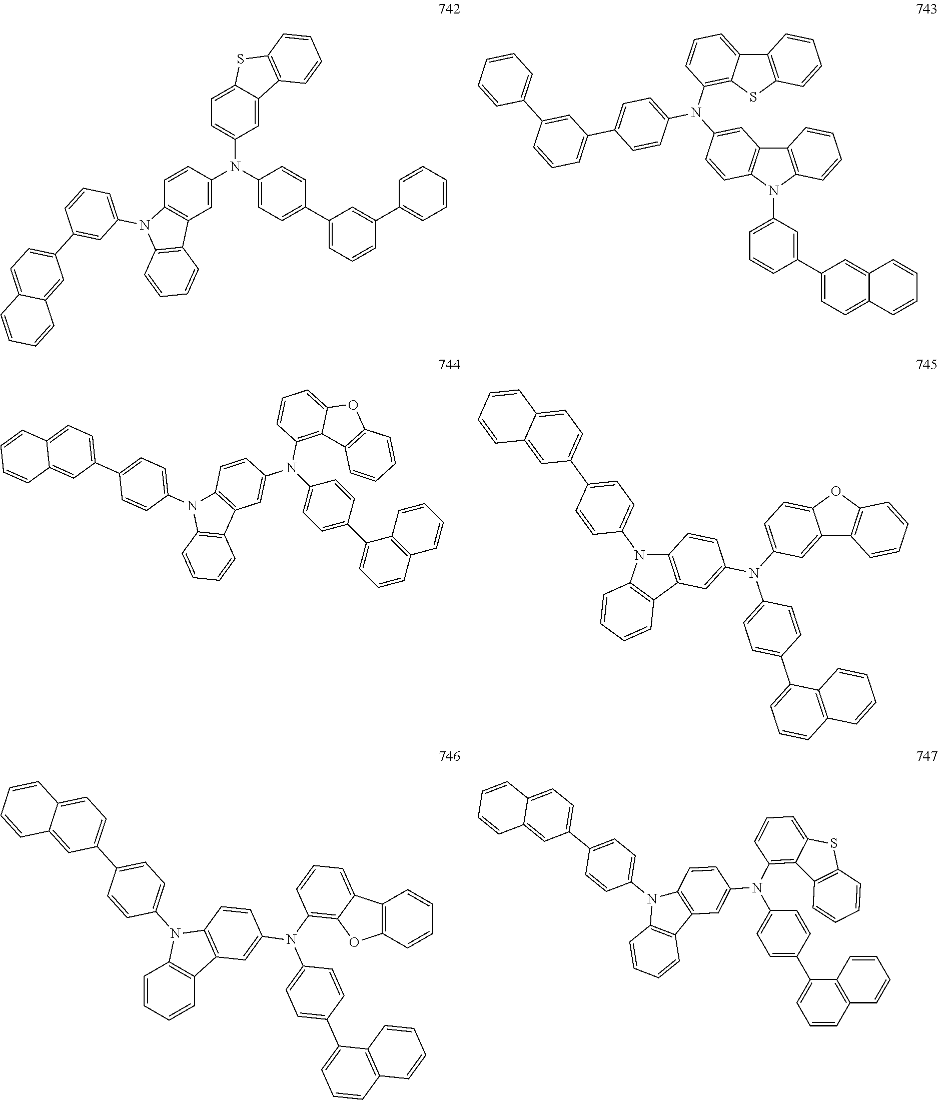 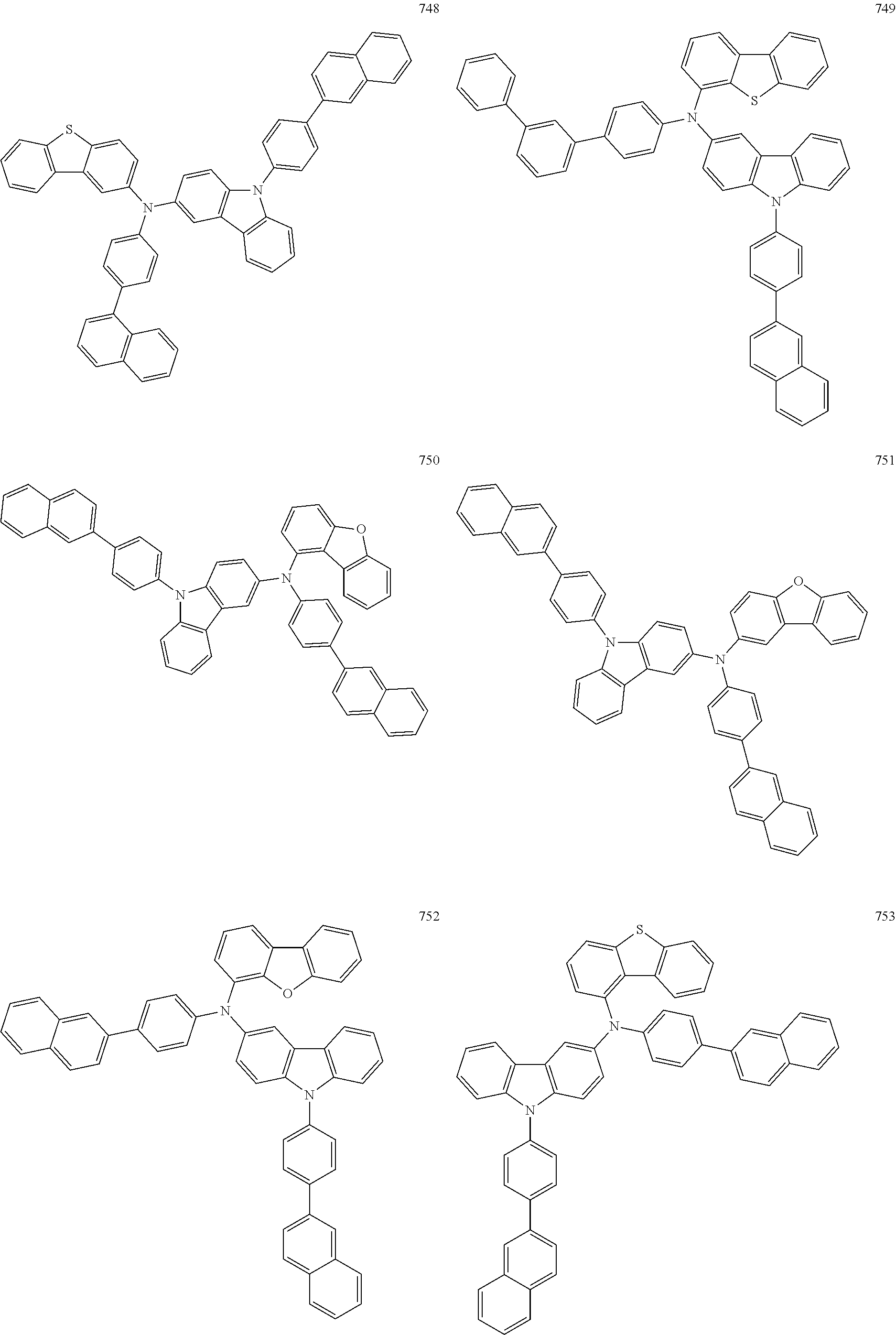 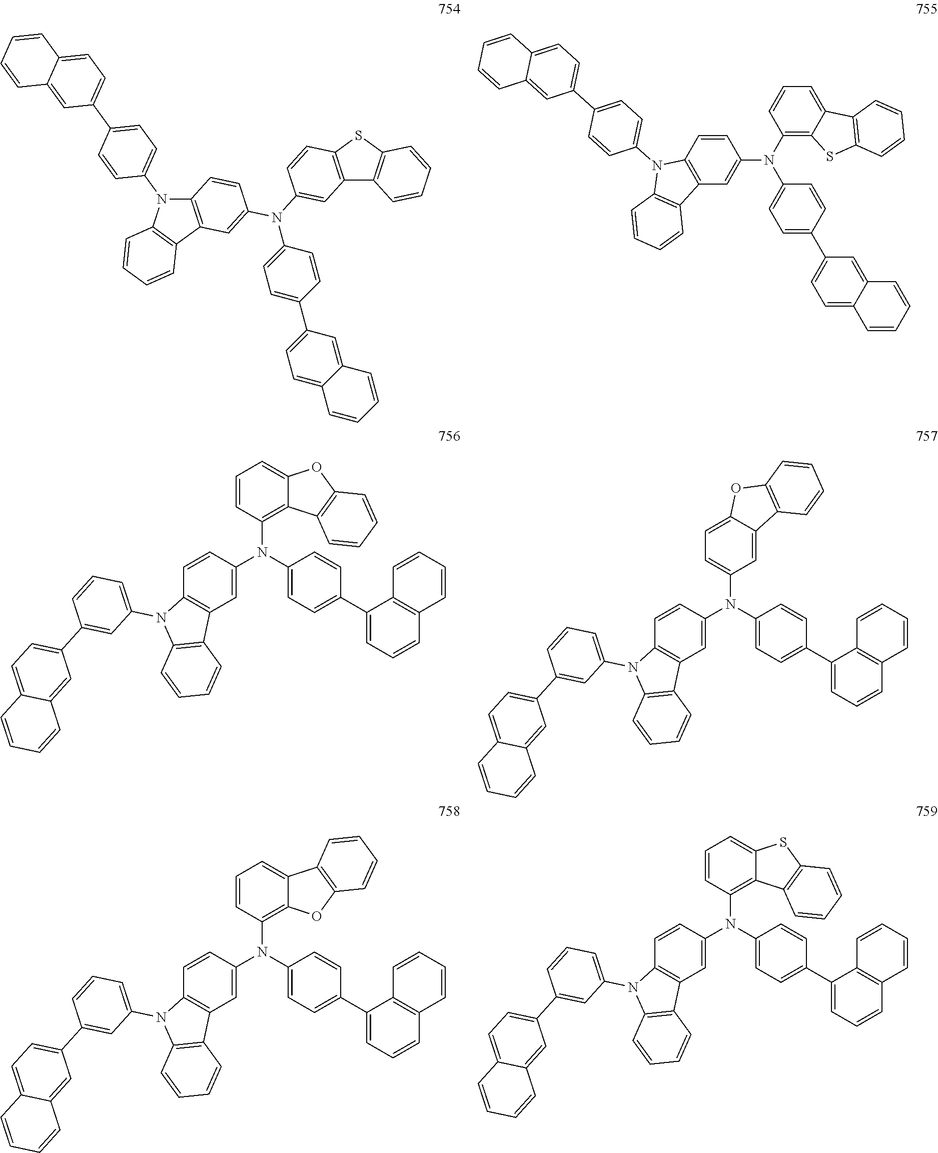 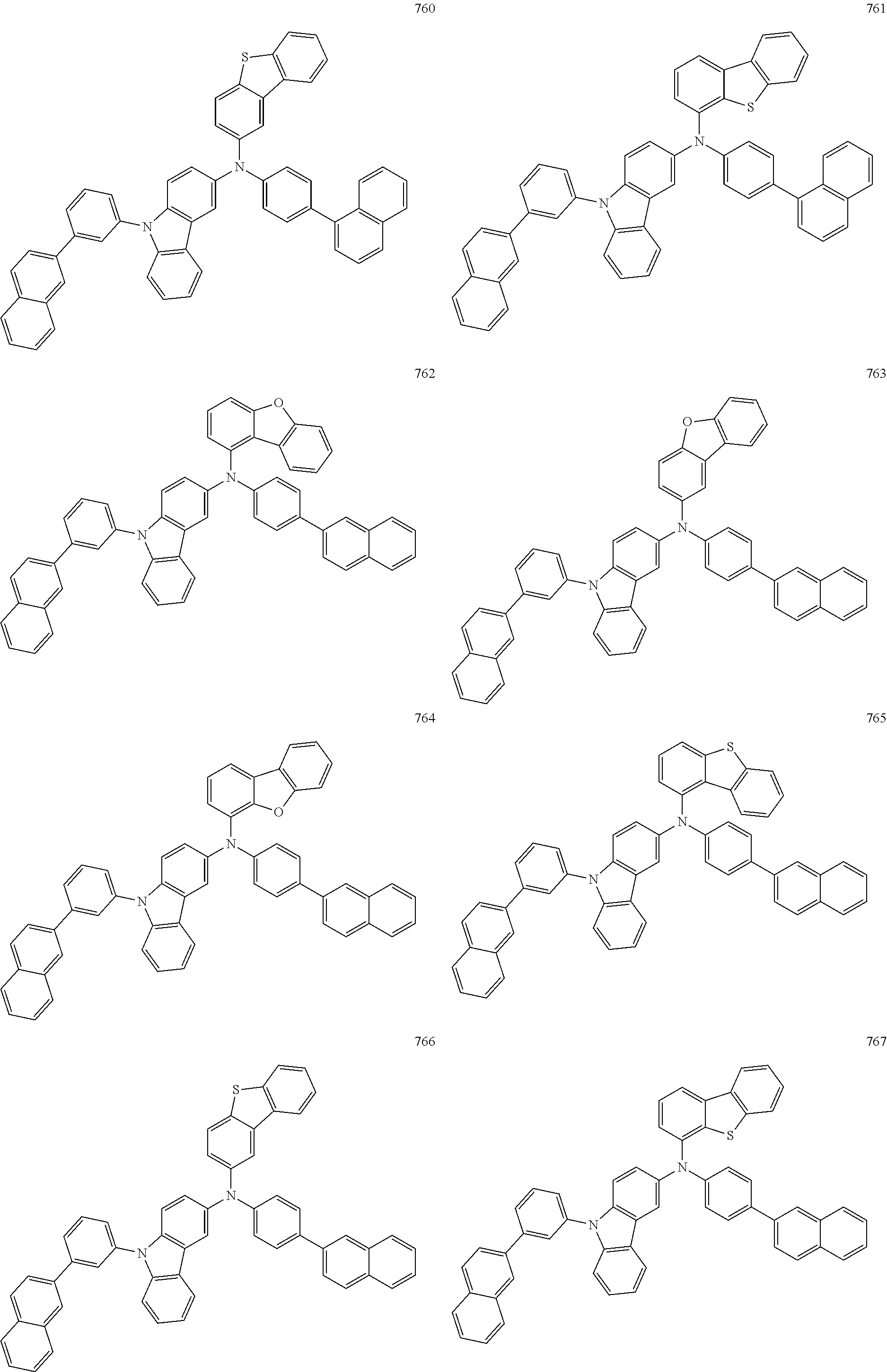 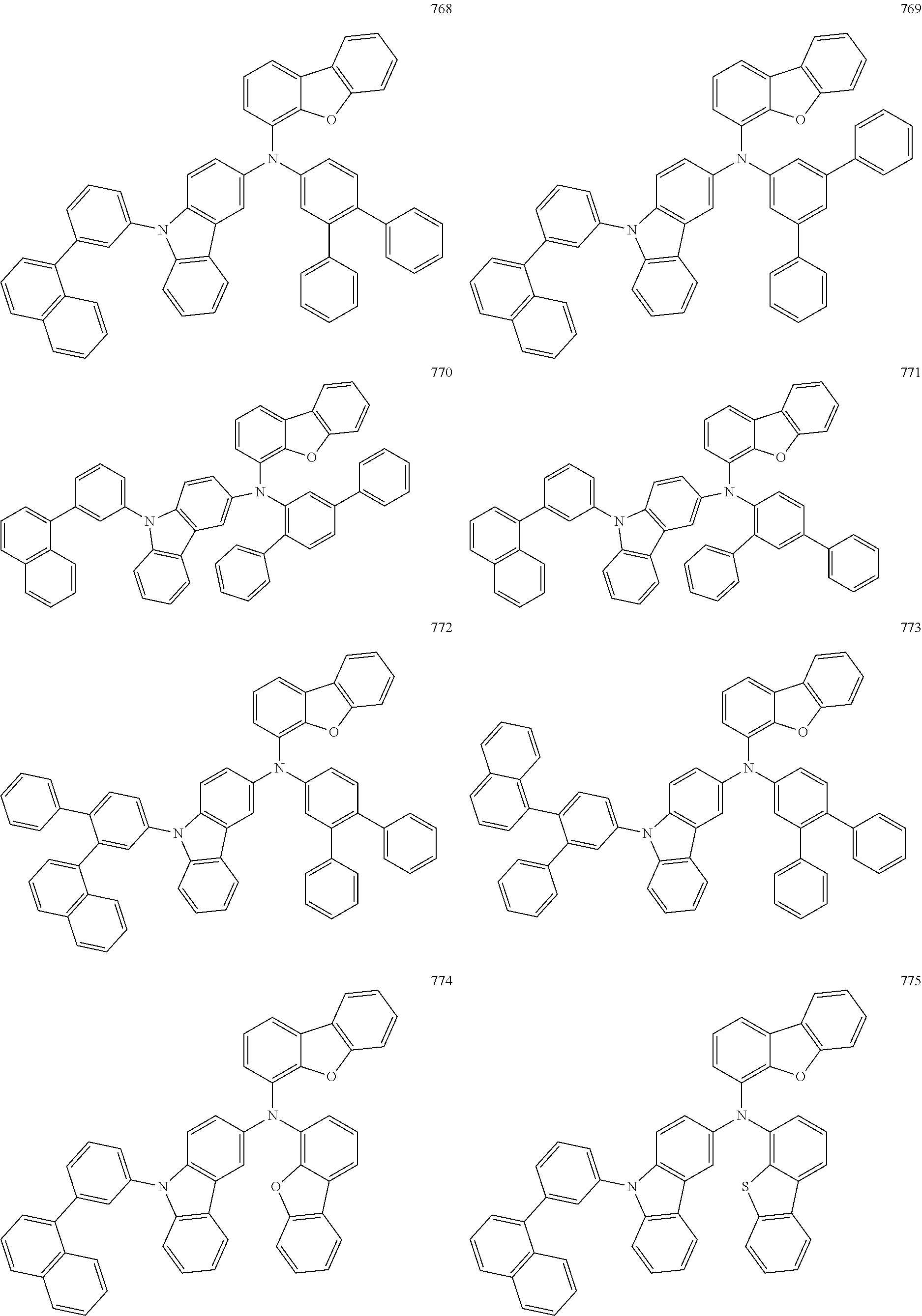 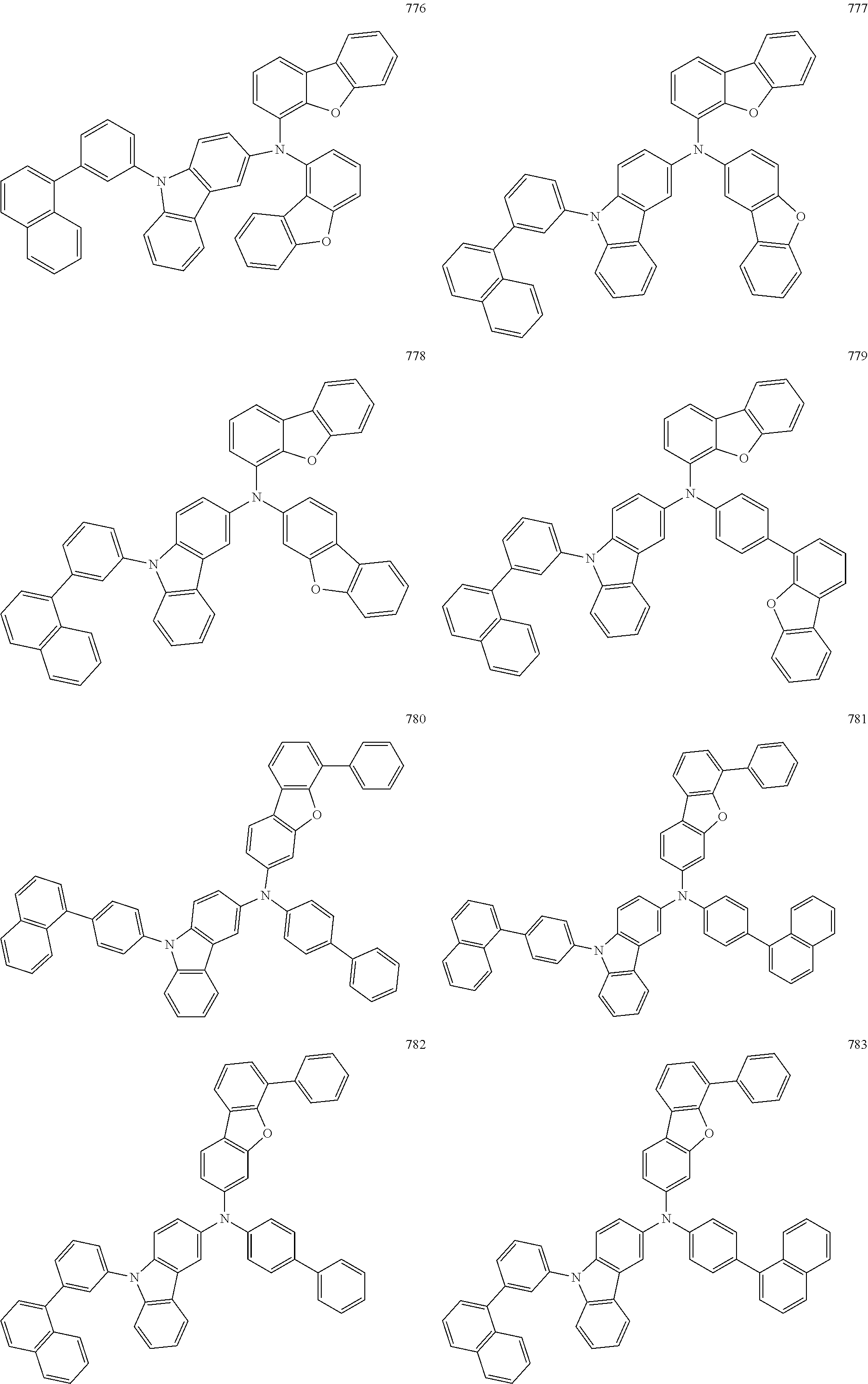 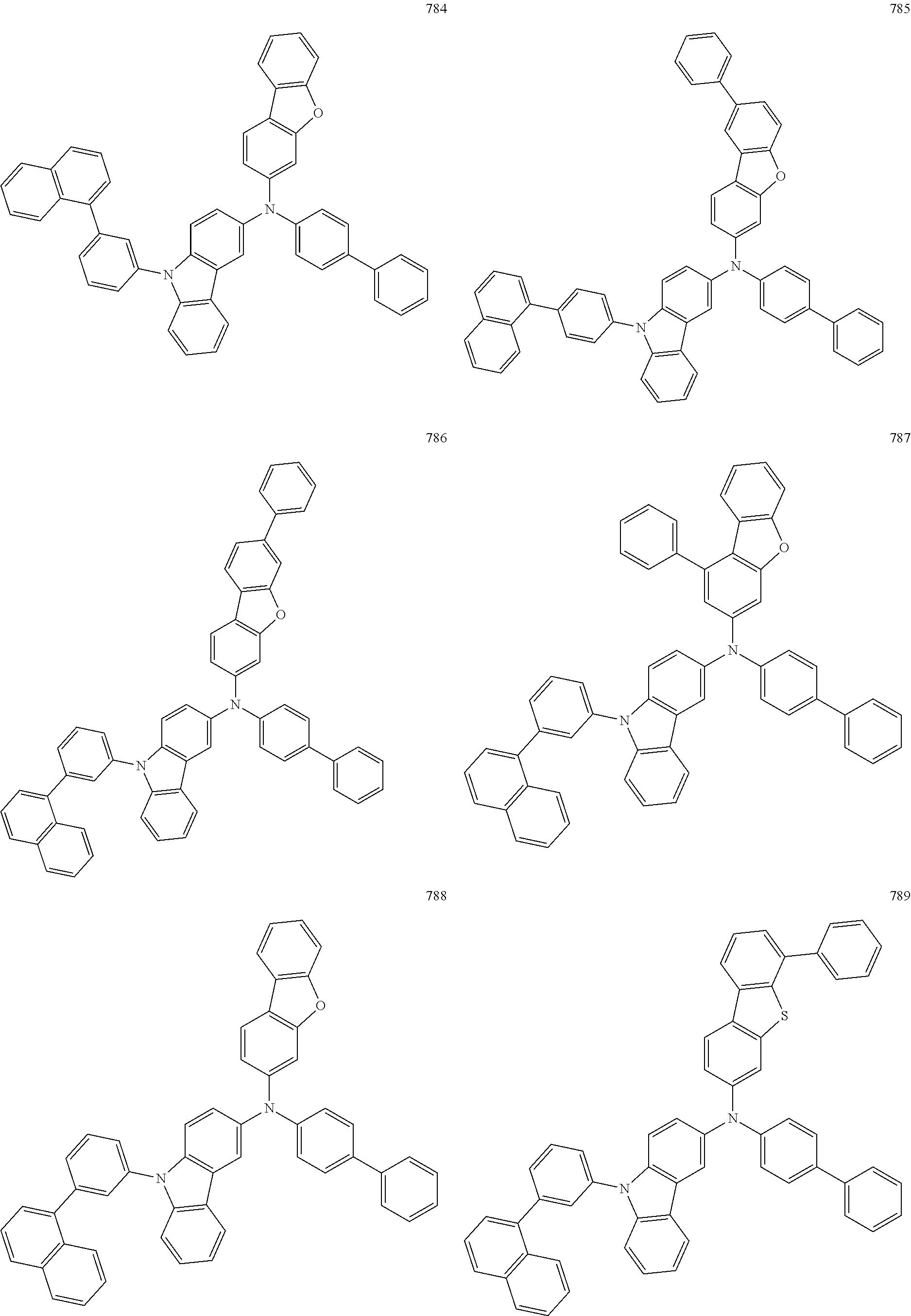 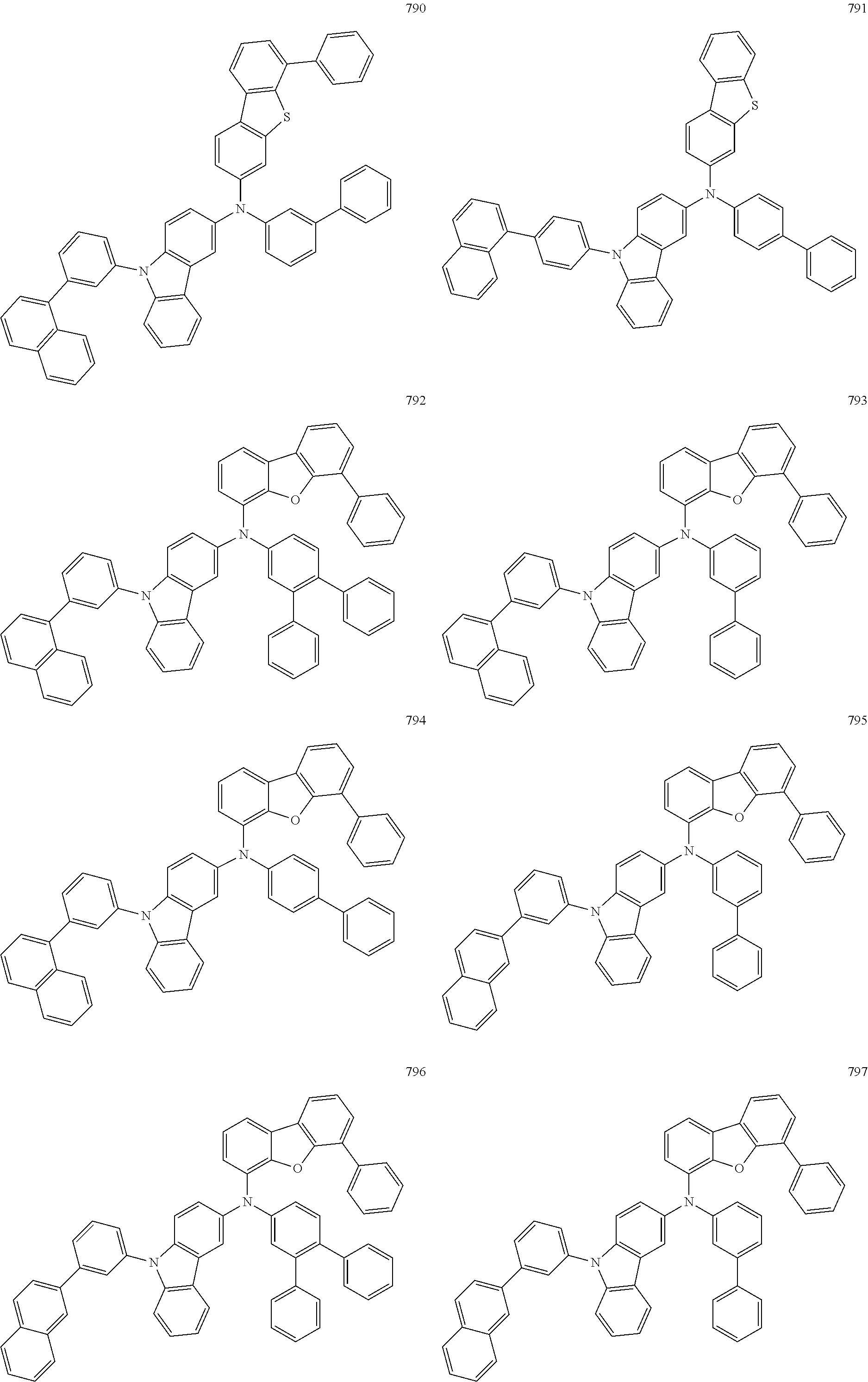 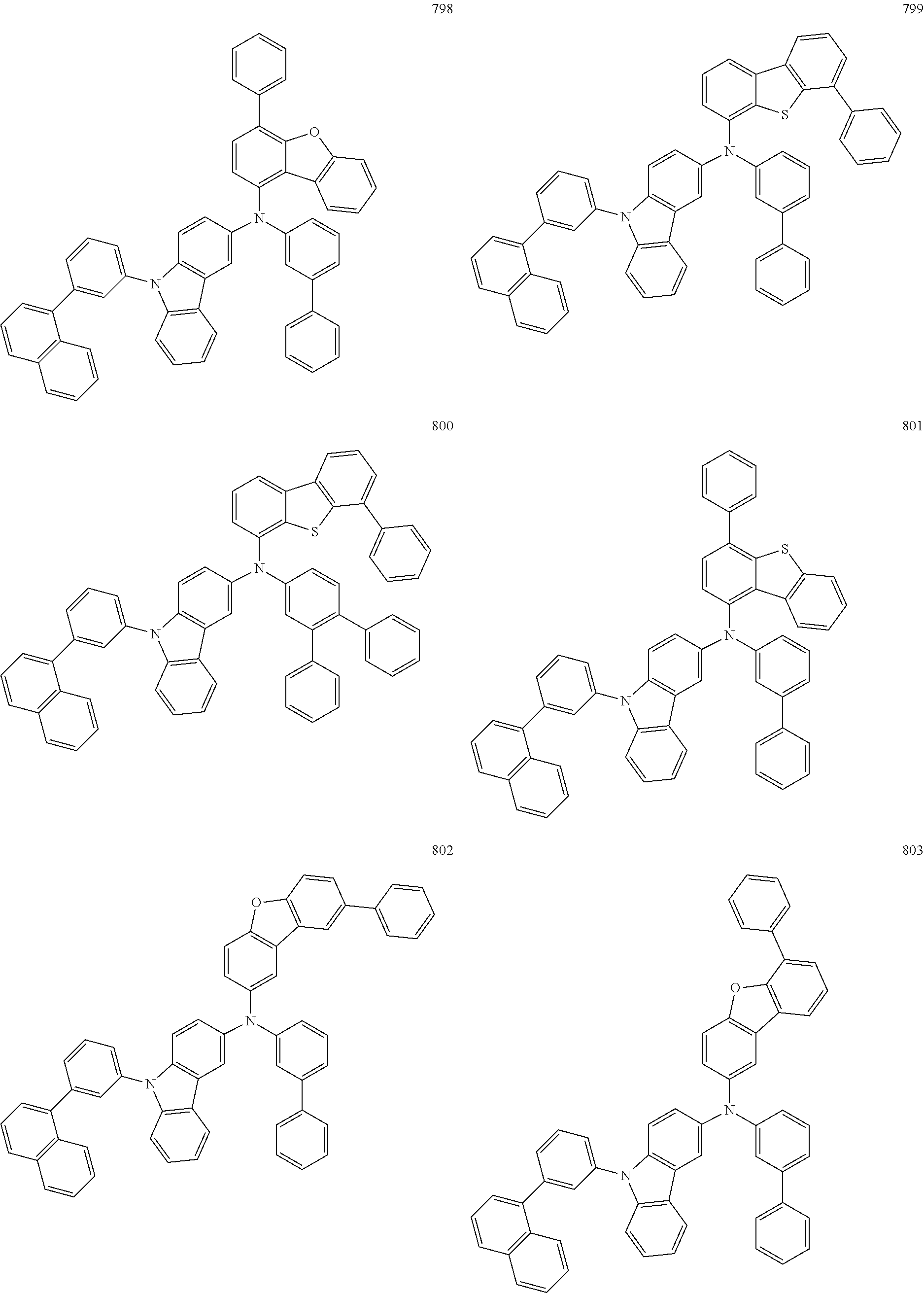 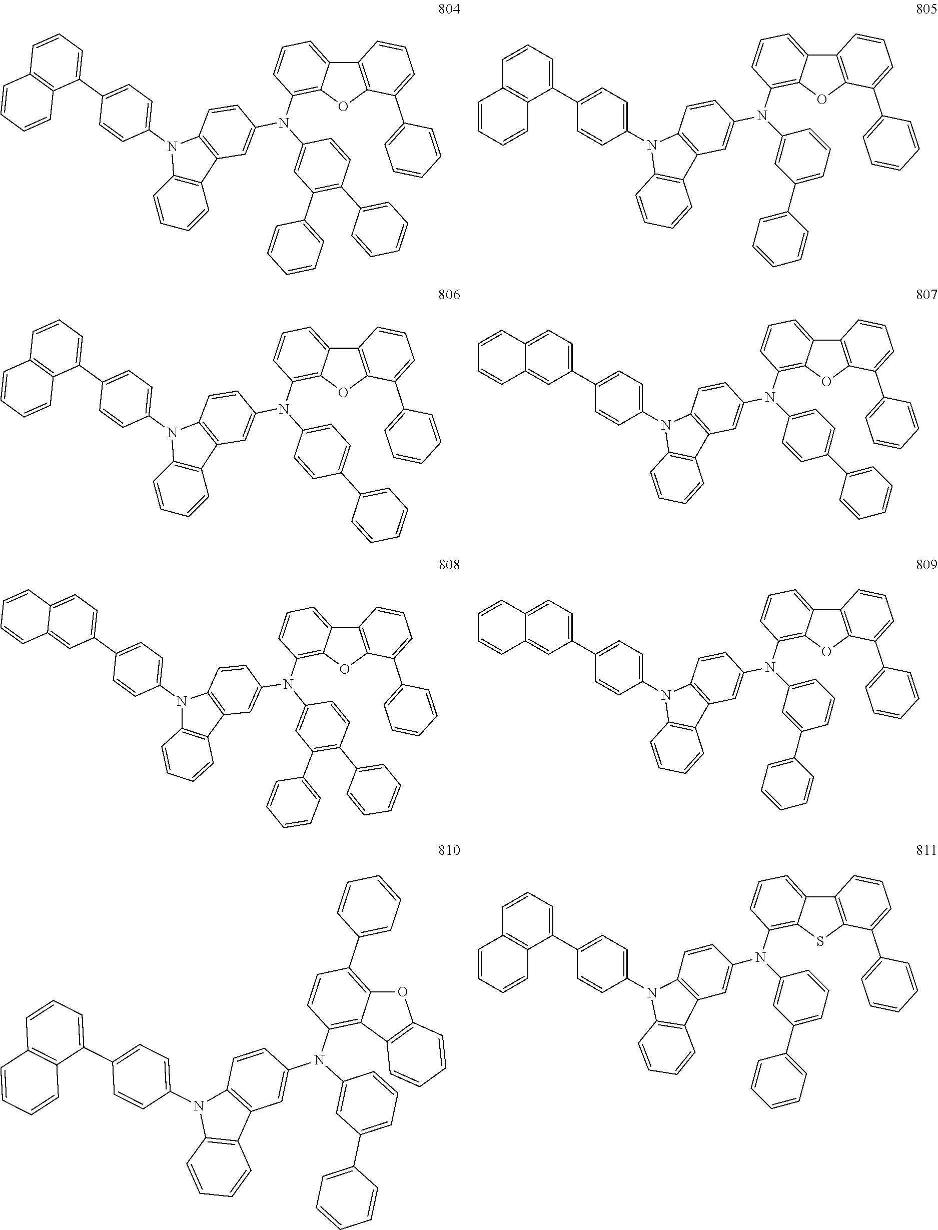 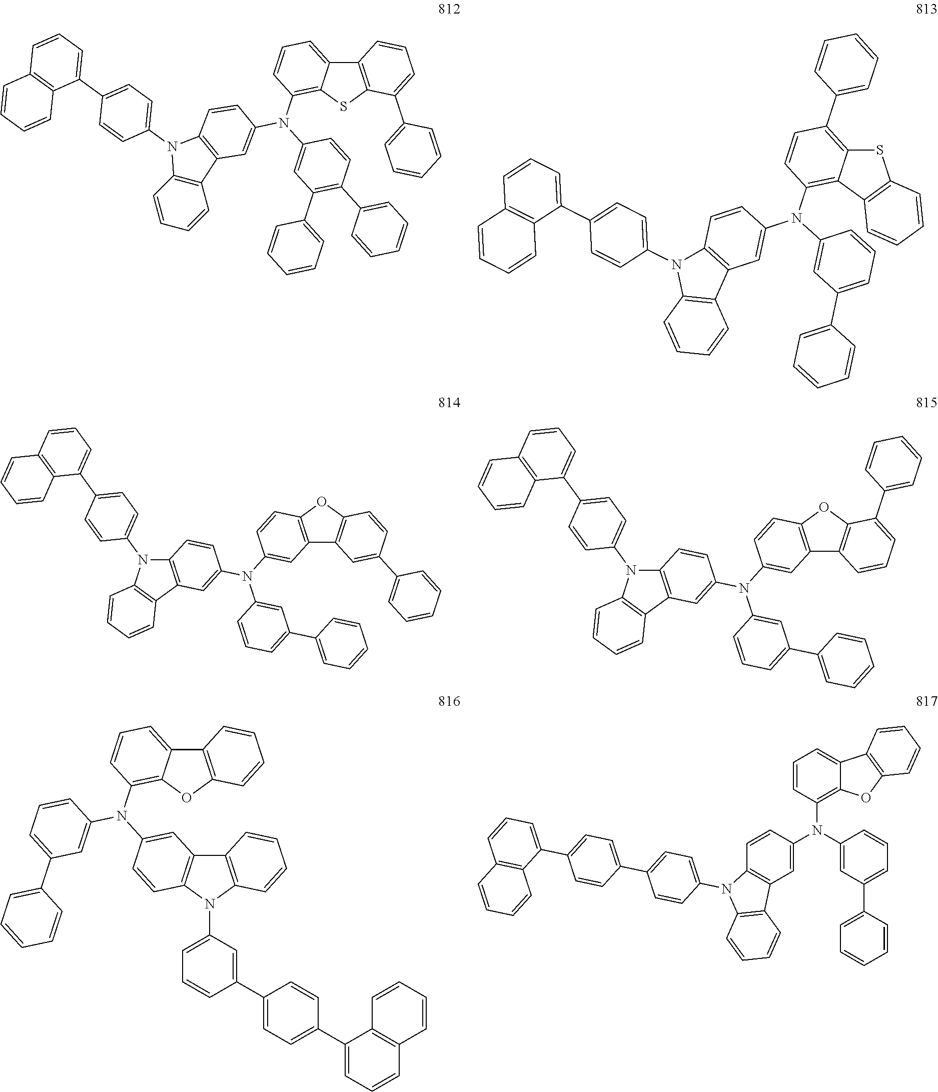 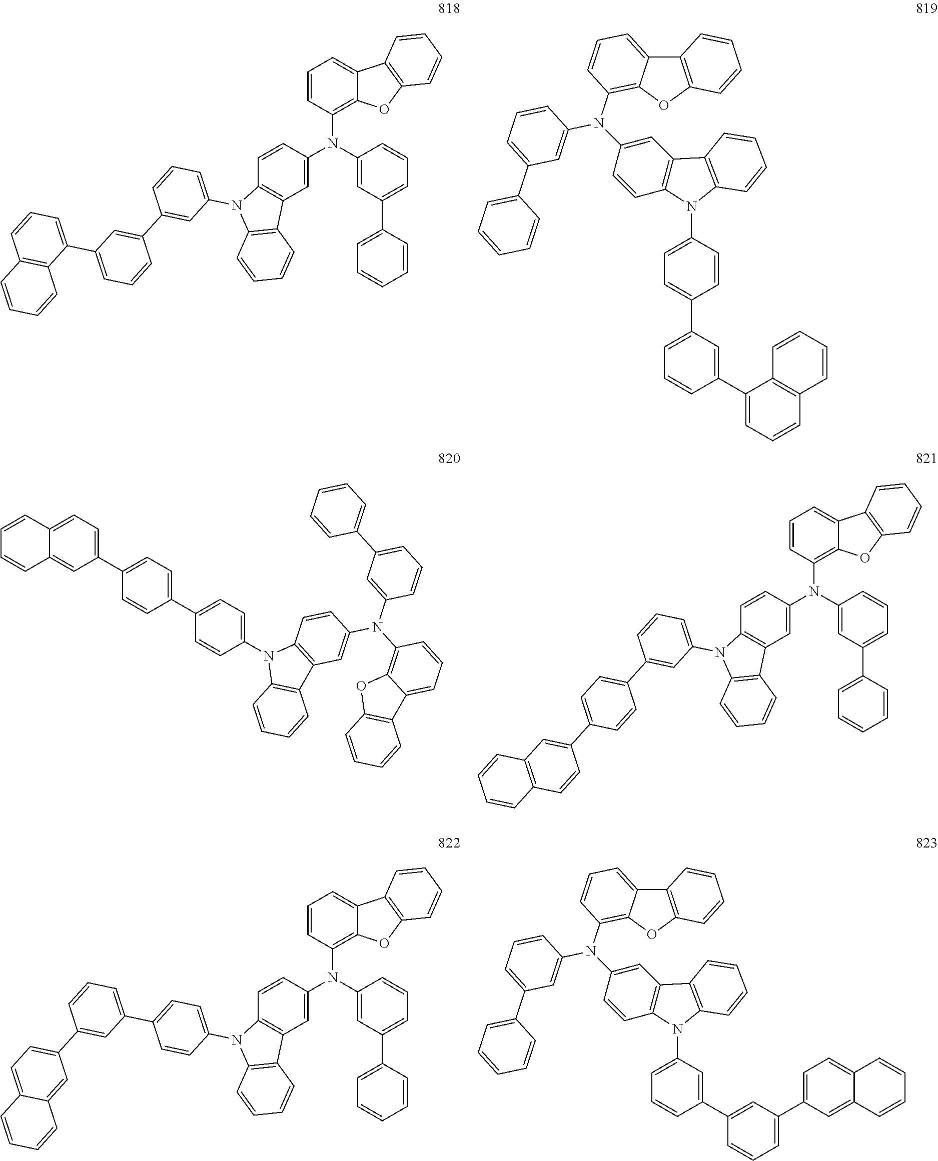 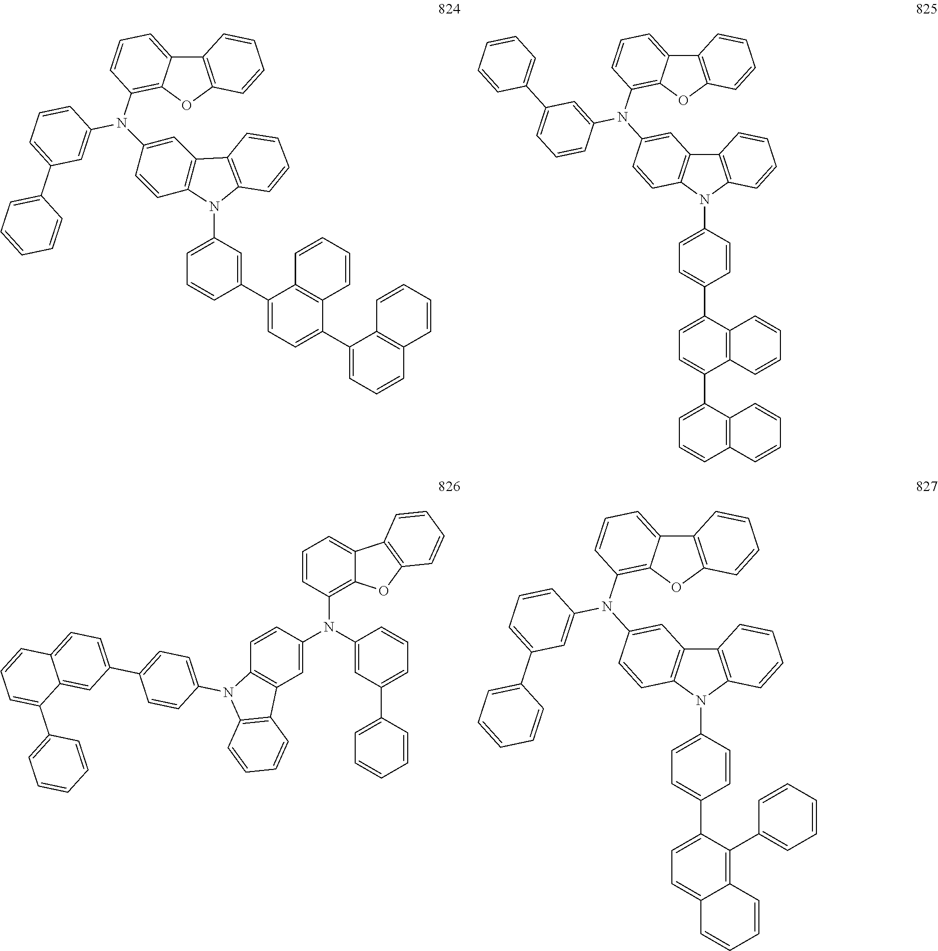 [SMILES: c1ccc(-c2cccc(N(c3ccc4c(c3)c3ccccc3n4-c3cccc(-c4ccc(-c5cccc6ccccc56)c5ccccc45)c3)c3cccc4c3oc3ccccc34)c2)cc1 ; c1ccc(-c2cccc(N(c3ccc4c(c3)c3ccccc3n4-c3ccc(-c4ccc(-c5cccc6ccccc56)c5ccccc45)cc3)c3cccc4c3oc3ccccc34)c2)cc1 ; c1ccc(-c2cccc(N(c3ccc4c(c3)c3ccccc3n4-c3ccc(-c4ccc5cccc(-c6ccccc6)c5c4)cc3)c3cccc4c3oc3ccccc34)c2)cc1 ; c1ccc(-c2cccc(N(c3ccc4c(c3)c3ccccc3n4-c3ccc(-c4ccc5ccccc5c4-c4ccccc4)cc3)c3cccc4c3oc3ccccc34)c2)cc1] 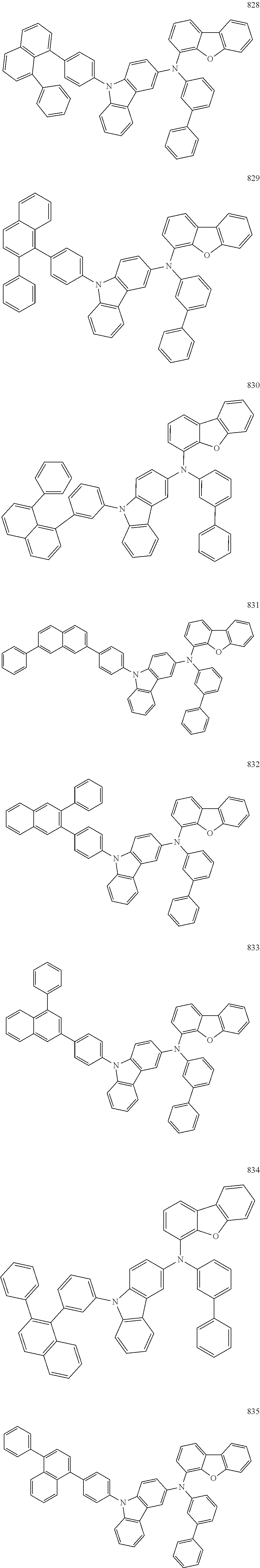 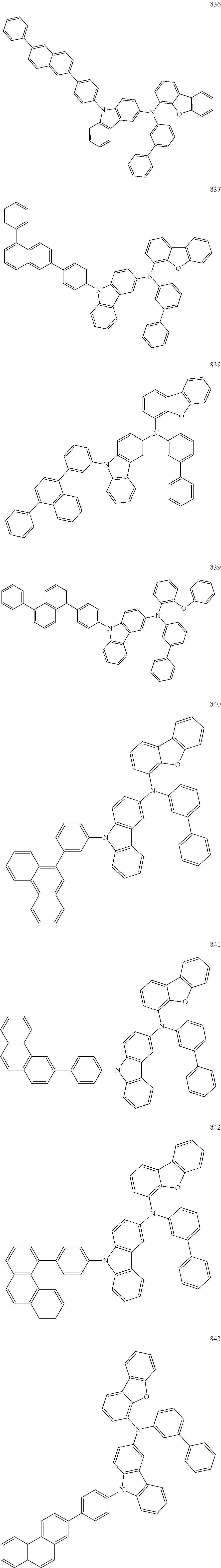 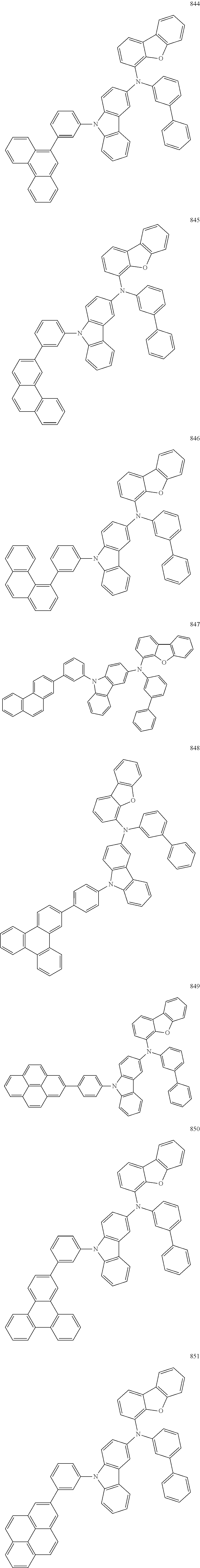 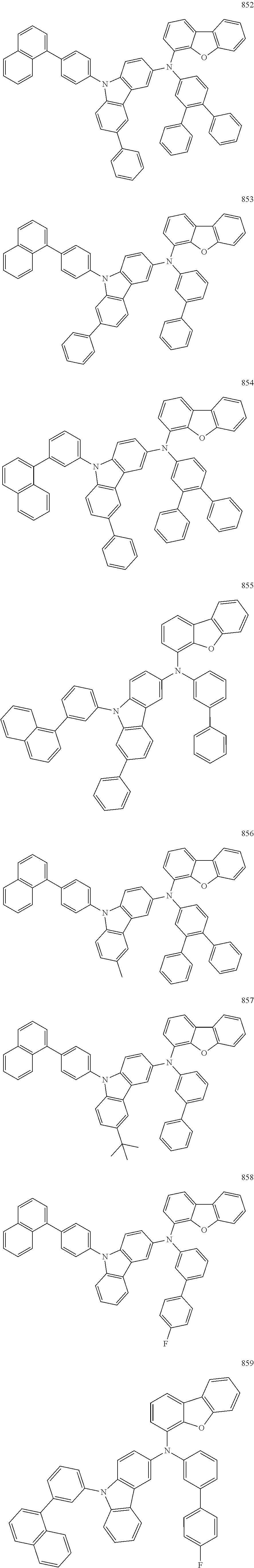 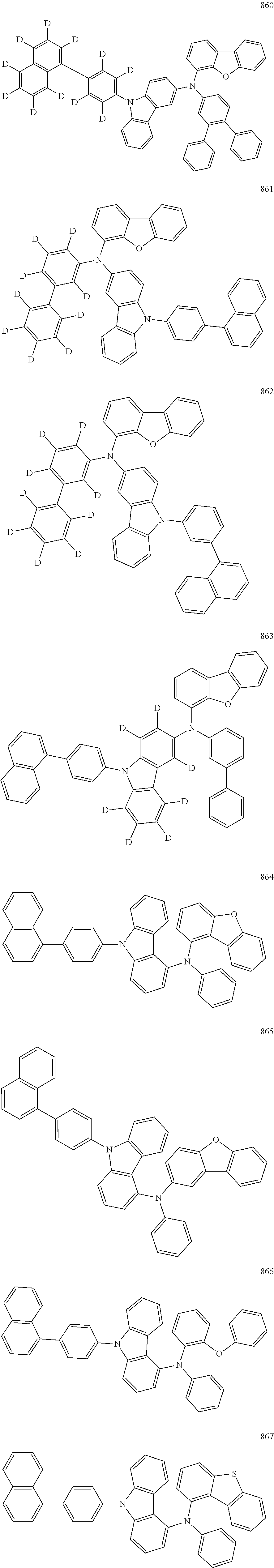 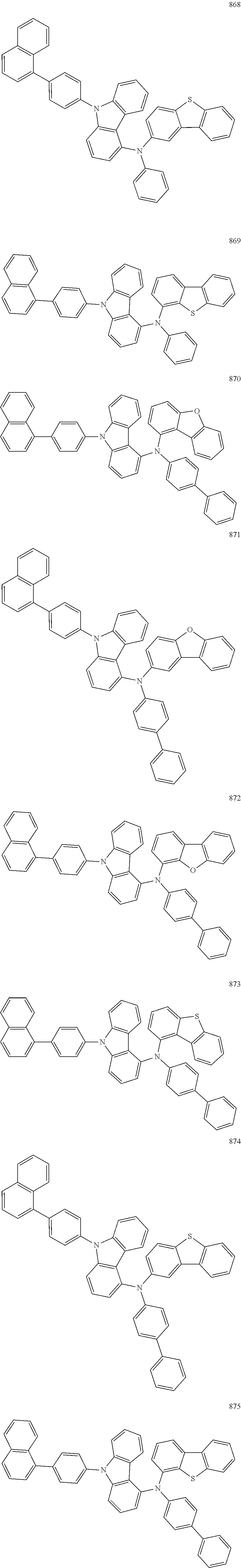 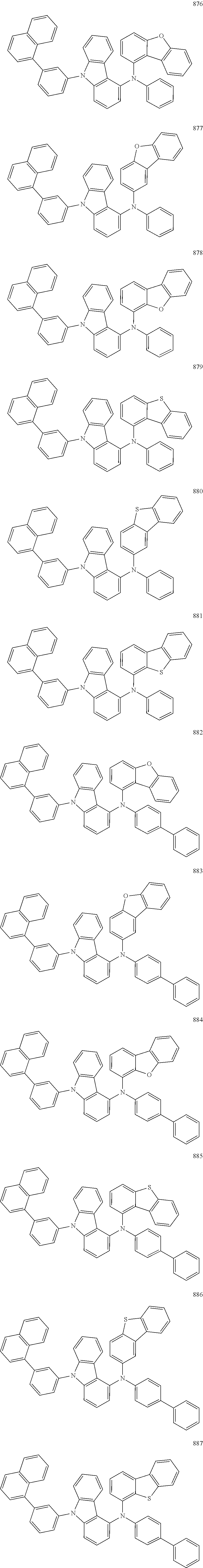 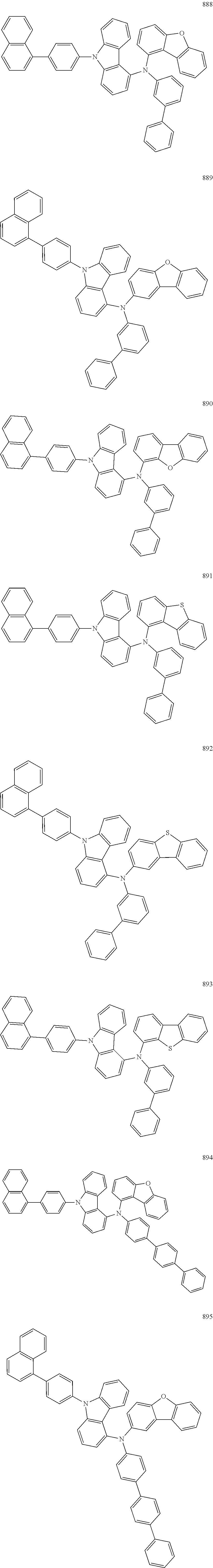 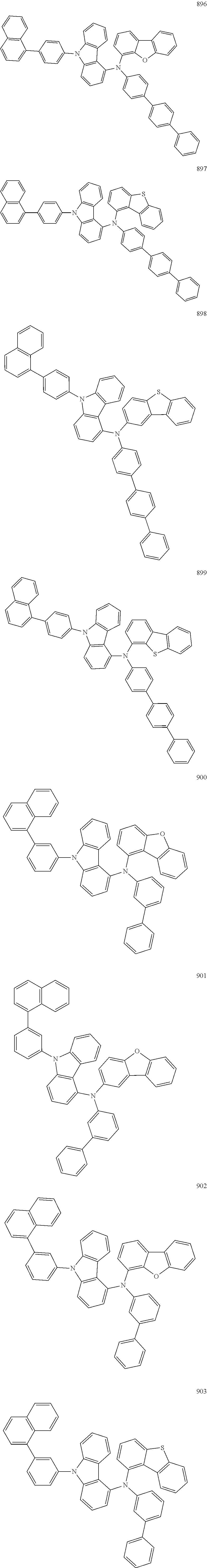 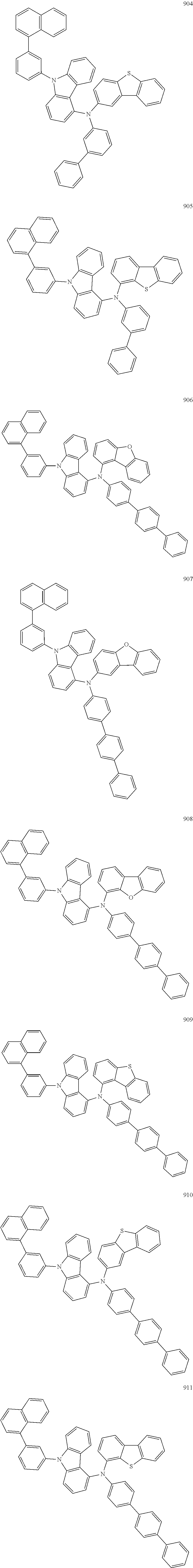 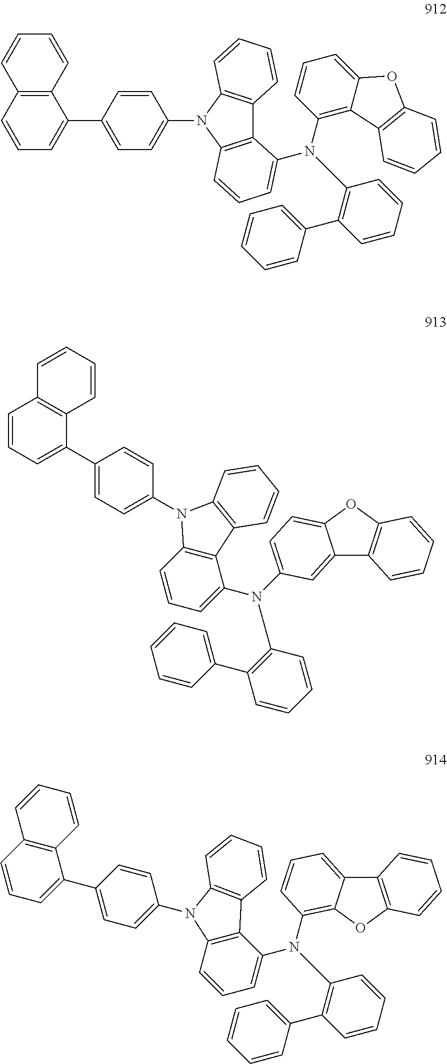 [SMILES: c1ccc(-c2ccccc2N(c2cccc3oc4ccccc4c23)c2cccc3c2c2ccccc2n3-c2ccc(-c3cccc4ccccc34)cc2)cc1 ; c1ccc(-c2ccccc2N(c2ccc3oc4ccccc4c3c2)c2cccc3c2c2ccccc2n3-c2ccc(-c3cccc4ccccc34)cc2)cc1 ; c1ccc(-c2ccccc2N(c2cccc3c2oc2ccccc23)c2cccc3c2c2ccccc2n3-c2ccc(-c3cccc4ccccc34)cc2)cc1] 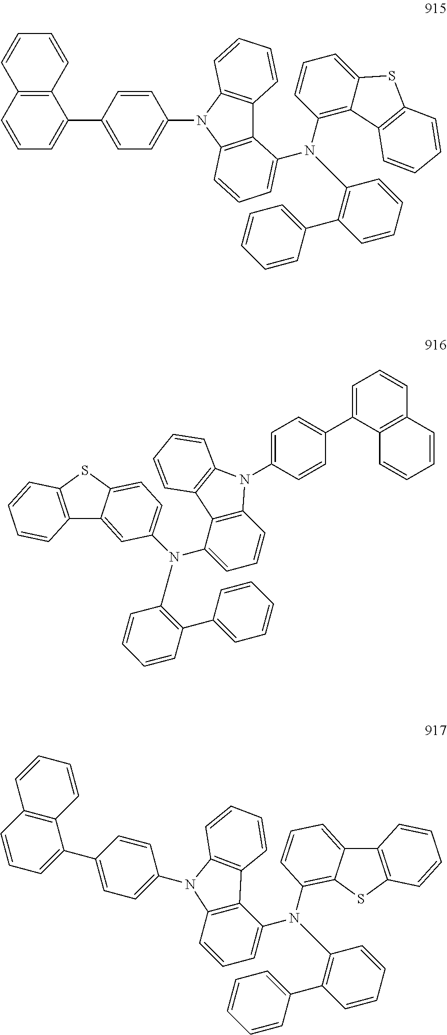 [SMILES: c1ccc(-c2ccccc2N(c2cccc3sc4ccccc4c23)c2cccc3c2c2ccccc2n3-c2ccc(-c3cccc4ccccc34)cc2)cc1 ; c1ccc(-c2ccccc2N(c2ccc3sc4ccccc4c3c2)c2cccc3c2c2ccccc2n3-c2ccc(-c3cccc4ccccc34)cc2)cc1 ; c1ccc(-c2ccccc2N(c2cccc3c2sc2ccccc23)c2cccc3c2c2ccccc2n3-c2ccc(-c3cccc4ccccc34)cc2)cc1] 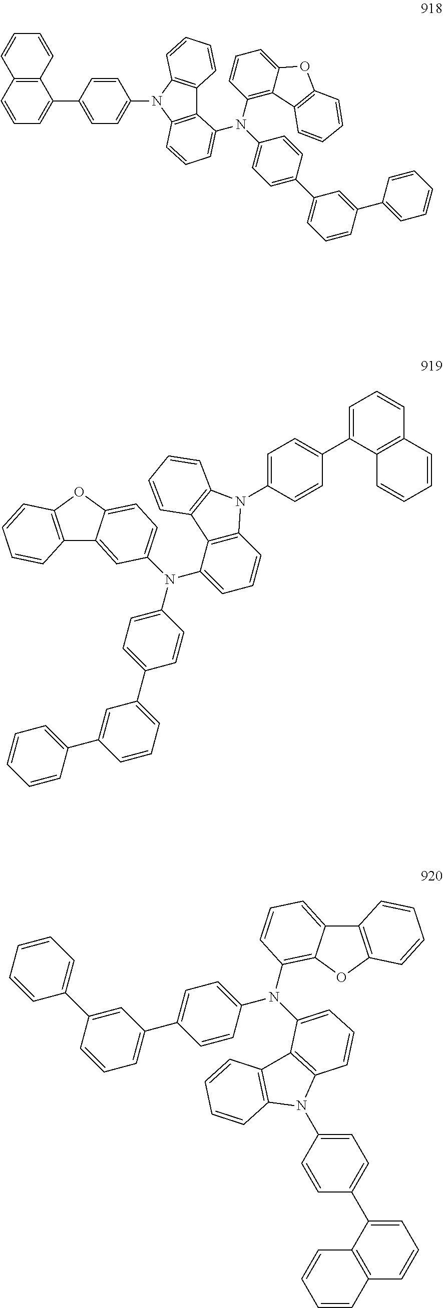 [SMILES: c1ccc(-c2cccc(-c3ccc(N(c4cccc5oc6ccccc6c45)c4cccc5c4c4ccccc4n5-c4ccc(-c5cccc6ccccc56)cc4)cc3)c2)cc1 ; c1ccc(-c2cccc(-c3ccc(N(c4ccc5oc6ccccc6c5c4)c4cccc5c4c4ccccc4n5-c4ccc(-c5cccc6ccccc56)cc4)cc3)c2)cc1 ; c1ccc(-c2cccc(-c3ccc(N(c4cccc5c4oc4ccccc45)c4cccc5c4c4ccccc4n5-c4ccc(-c5cccc6ccccc56)cc4)cc3)c2)cc1] 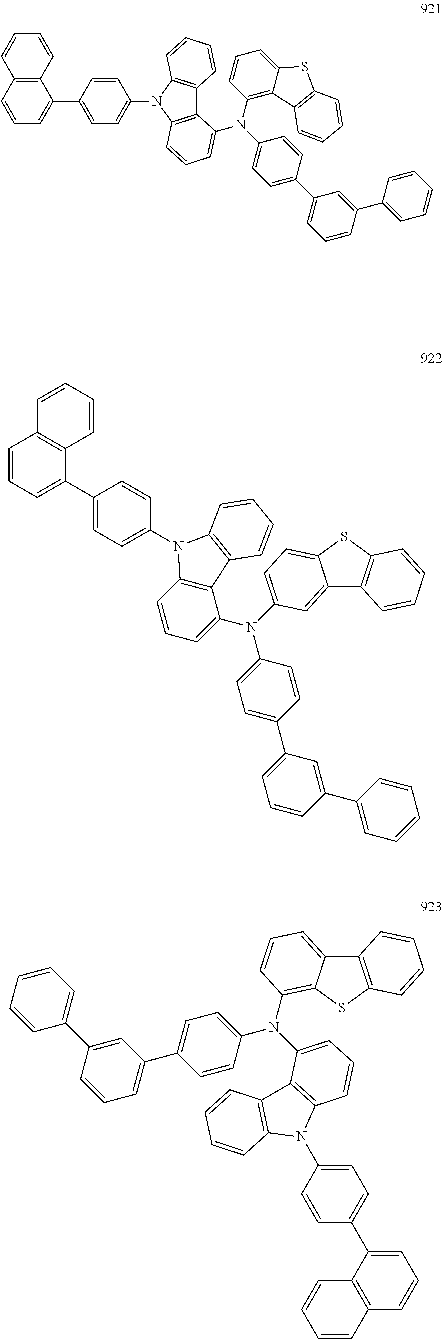 [SMILES: c1ccc(-c2cccc(-c3ccc(N(c4cccc5sc6ccccc6c45)c4cccc5c4c4ccccc4n5-c4ccc(-c5cccc6ccccc56)cc4)cc3)c2)cc1 ; c1ccc(-c2cccc(-c3ccc(N(c4ccc5sc6ccccc6c5c4)c4cccc5c4c4ccccc4n5-c4ccc(-c5cccc6ccccc56)cc4)cc3)c2)cc1 ; c1ccc(-c2cccc(-c3ccc(N(c4cccc5c4sc4ccccc45)c4cccc5c4c4ccccc4n5-c4ccc(-c5cccc6ccccc56)cc4)cc3)c2)cc1] 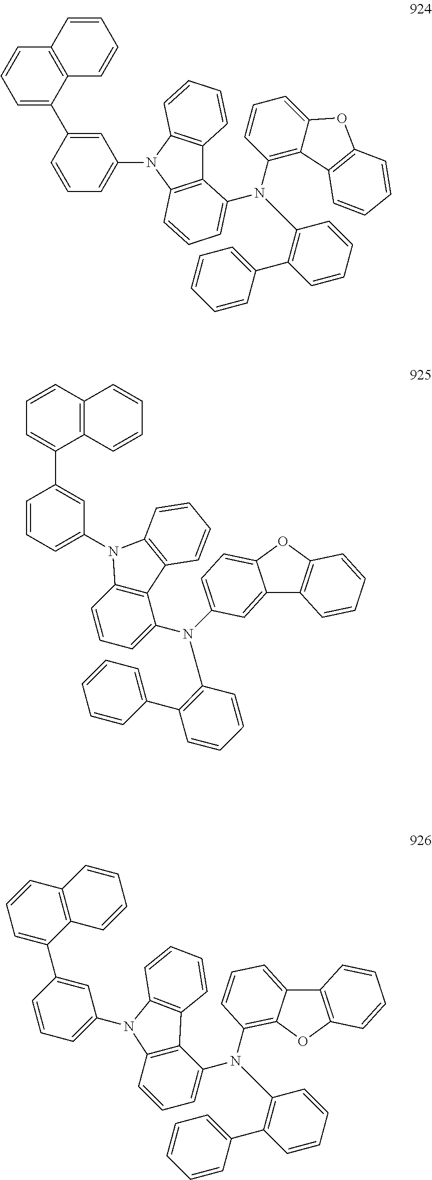 [SMILES: c1ccc(-c2ccccc2N(c2cccc3oc4ccccc4c23)c2cccc3c2c2ccccc2n3-c2cccc(-c3cccc4ccccc34)c2)cc1 ; c1ccc(-c2ccccc2N(c2ccc3oc4ccccc4c3c2)c2cccc3c2c2ccccc2n3-c2cccc(-c3cccc4ccccc34)c2)cc1 ; c1ccc(-c2ccccc2N(c2cccc3c2oc2ccccc23)c2cccc3c2c2ccccc2n3-c2cccc(-c3cccc4ccccc34)c2)cc1] 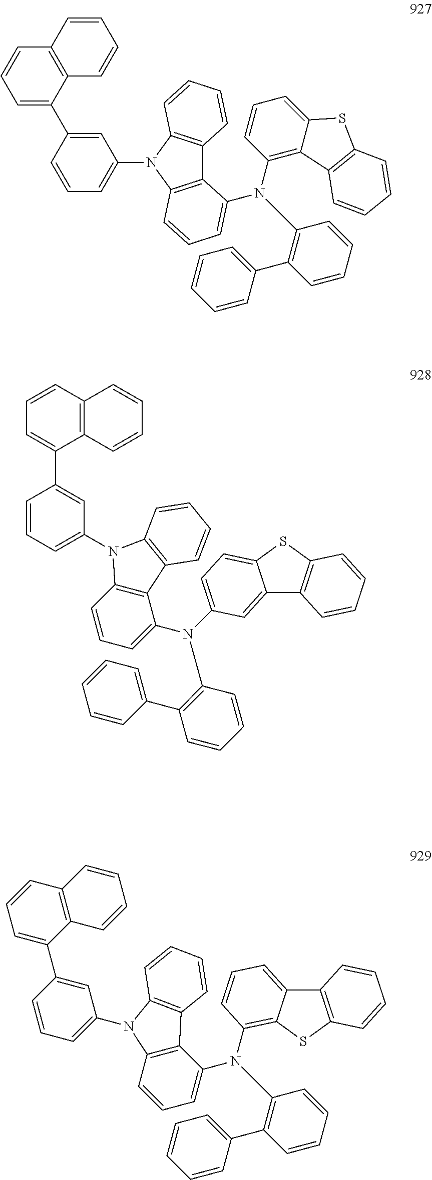 [SMILES: c1ccc(-c2ccccc2N(c2cccc3sc4ccccc4c23)c2cccc3c2c2ccccc2n3-c2cccc(-c3cccc4ccccc34)c2)cc1 ; c1ccc(-c2ccccc2N(c2ccc3sc4ccccc4c3c2)c2cccc3c2c2ccccc2n3-c2cccc(-c3cccc4ccccc34)c2)cc1 ; c1ccc(-c2ccccc2N(c2cccc3c2sc2ccccc23)c2cccc3c2c2ccccc2n3-c2cccc(-c3cccc4ccccc34)c2)cc1] 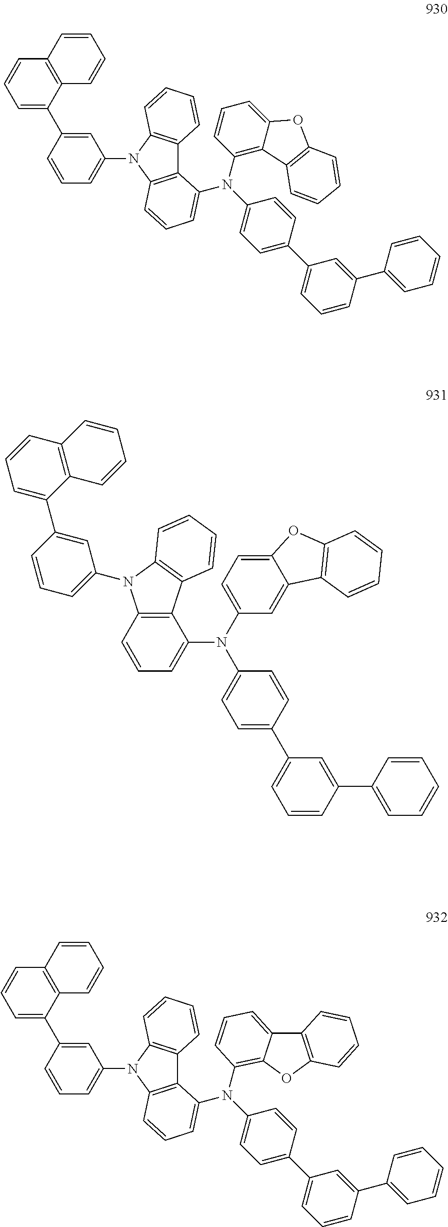 [SMILES: c1ccc(-c2cccc(-c3ccc(N(c4cccc5oc6ccccc6c45)c4cccc5c4c4ccccc4n5-c4cccc(-c5cccc6ccccc56)c4)cc3)c2)cc1 ; c1ccc(-c2cccc(-c3ccc(N(c4ccc5oc6ccccc6c5c4)c4cccc5c4c4ccccc4n5-c4cccc(-c5cccc6ccccc56)c4)cc3)c2)cc1 ; c1ccc(-c2cccc(-c3ccc(N(c4cccc5c4oc4ccccc45)c4cccc5c4c4ccccc4n5-c4cccc(-c5cccc6ccccc56)c4)cc3)c2)cc1] 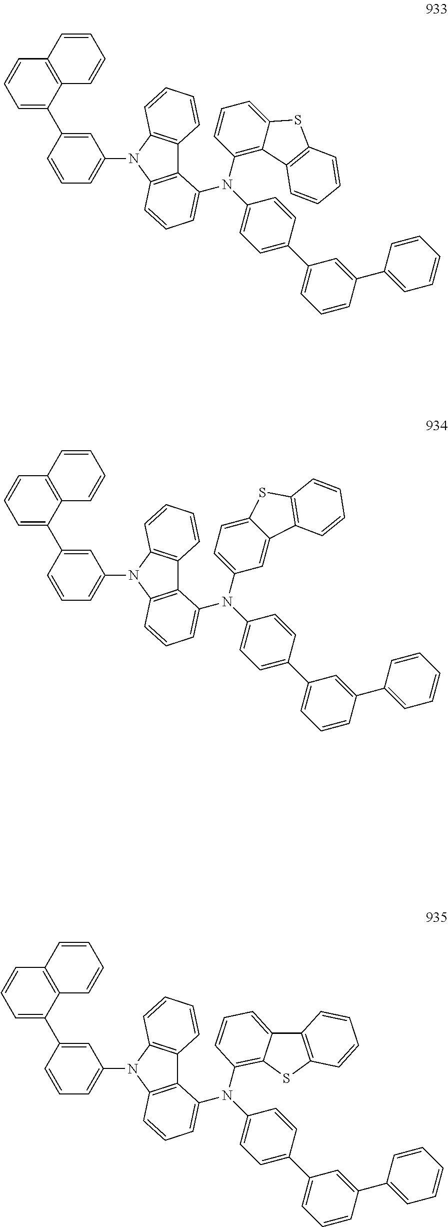 [SMILES: c1ccc(-c2cccc(-c3ccc(N(c4cccc5sc6ccccc6c45)c4cccc5c4c4ccccc4n5-c4cccc(-c5cccc6ccccc56)c4)cc3)c2)cc1 ; c1ccc(-c2cccc(-c3ccc(N(c4ccc5sc6ccccc6c5c4)c4cccc5c4c4ccccc4n5-c4cccc(-c5cccc6ccccc56)c4)cc3)c2)cc1 ; c1ccc(-c2cccc(-c3ccc(N(c4cccc5c4sc4ccccc45)c4cccc5c4c4ccccc4n5-c4cccc(-c5cccc6ccccc56)c4)cc3)c2)cc1] 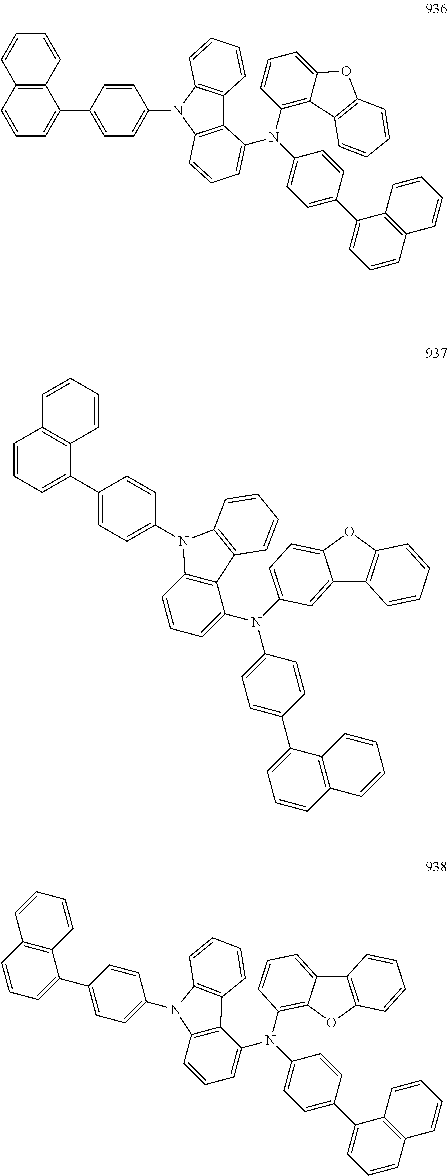 [SMILES: c1ccc2c(-c3ccc(N(c4cccc5oc6ccccc6c45)c4cccc5c4c4ccccc4n5-c4ccc(-c5cccc6ccccc56)cc4)cc3)cccc2c1 ; c1ccc2c(-c3ccc(N(c4ccc5oc6ccccc6c5c4)c4cccc5c4c4ccccc4n5-c4ccc(-c5cccc6ccccc56)cc4)cc3)cccc2c1 ; c1ccc2c(-c3ccc(N(c4cccc5c4oc4ccccc45)c4cccc5c4c4ccccc4n5-c4ccc(-c5cccc6ccccc56)cc4)cc3)cccc2c1] 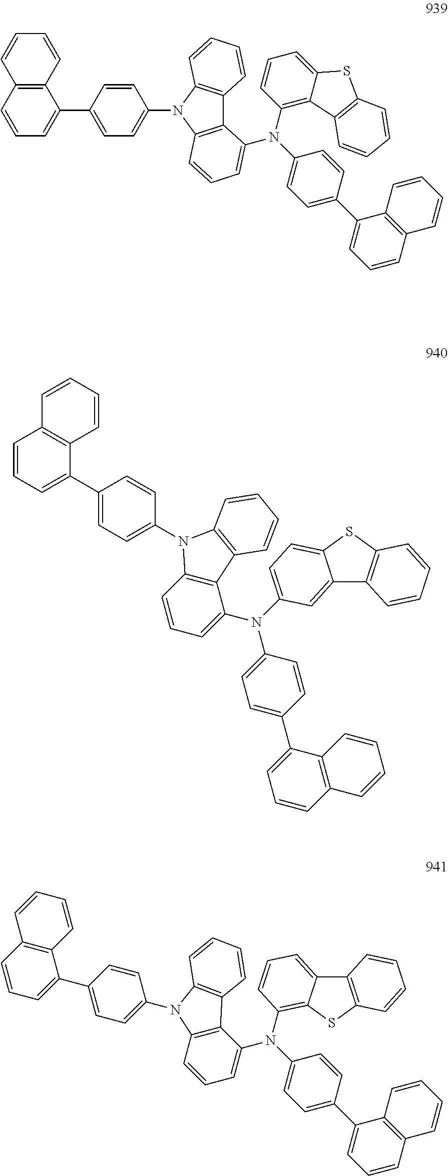 [SMILES: c1ccc2c(-c3ccc(N(c4cccc5sc6ccccc6c45)c4cccc5c4c4ccccc4n5-c4ccc(-c5cccc6ccccc56)cc4)cc3)cccc2c1 ; c1ccc2c(-c3ccc(N(c4ccc5sc6ccccc6c5c4)c4cccc5c4c4ccccc4n5-c4ccc(-c5cccc6ccccc56)cc4)cc3)cccc2c1 ; c1ccc2c(-c3ccc(N(c4cccc5c4sc4ccccc45)c4cccc5c4c4ccccc4n5-c4ccc(-c5cccc6ccccc56)cc4)cc3)cccc2c1] 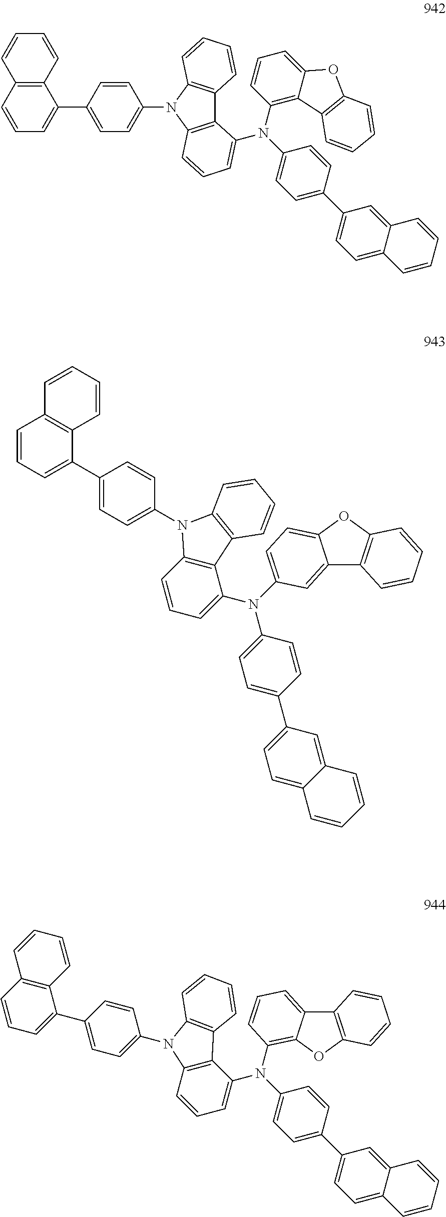 [SMILES: c1ccc2cc(-c3ccc(N(c4cccc5oc6ccccc6c45)c4cccc5c4c4ccccc4n5-c4ccc(-c5cccc6ccccc56)cc4)cc3)ccc2c1 ; c1ccc2cc(-c3ccc(N(c4ccc5oc6ccccc6c5c4)c4cccc5c4c4ccccc4n5-c4ccc(-c5cccc6ccccc56)cc4)cc3)ccc2c1 ; c1ccc2cc(-c3ccc(N(c4cccc5c4oc4ccccc45)c4cccc5c4c4ccccc4n5-c4ccc(-c5cccc6ccccc56)cc4)cc3)ccc2c1] 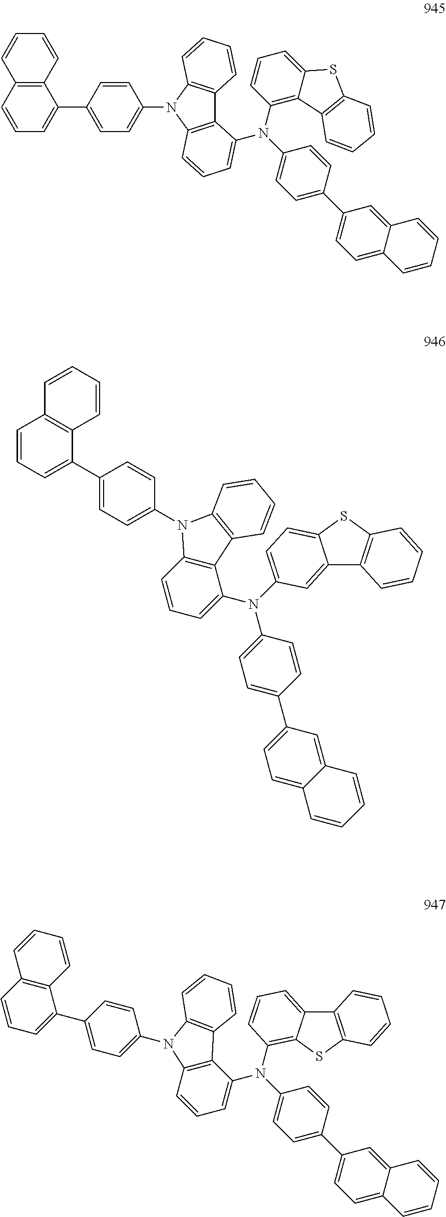 [SMILES: c1ccc2cc(-c3ccc(N(c4cccc5sc6ccccc6c45)c4cccc5c4c4ccccc4n5-c4ccc(-c5cccc6ccccc56)cc4)cc3)ccc2c1 ; c1ccc2cc(-c3ccc(N(c4ccc5sc6ccccc6c5c4)c4cccc5c4c4ccccc4n5-c4ccc(-c5cccc6ccccc56)cc4)cc3)ccc2c1 ; c1ccc2cc(-c3ccc(N(c4cccc5c4sc4ccccc45)c4cccc5c4c4ccccc4n5-c4ccc(-c5cccc6ccccc56)cc4)cc3)ccc2c1] 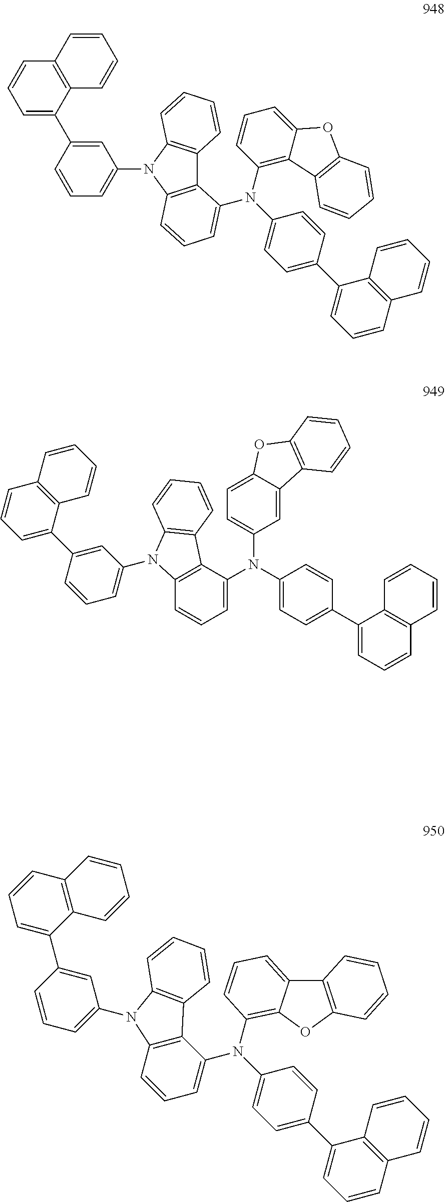 [SMILES: c1cc(-c2cccc3ccccc23)cc(-n2c3ccccc3c3c(N(c4ccc(-c5cccc6ccccc56)cc4)c4cccc5oc6ccccc6c45)cccc32)c1 ; c1cc(-c2cccc3ccccc23)cc(-n2c3ccccc3c3c(N(c4ccc(-c5cccc6ccccc56)cc4)c4ccc5oc6ccccc6c5c4)cccc32)c1 ; c1cc(-c2cccc3ccccc23)cc(-n2c3ccccc3c3c(N(c4ccc(-c5cccc6ccccc56)cc4)c4cccc5c4oc4ccccc45)cccc32)c1] 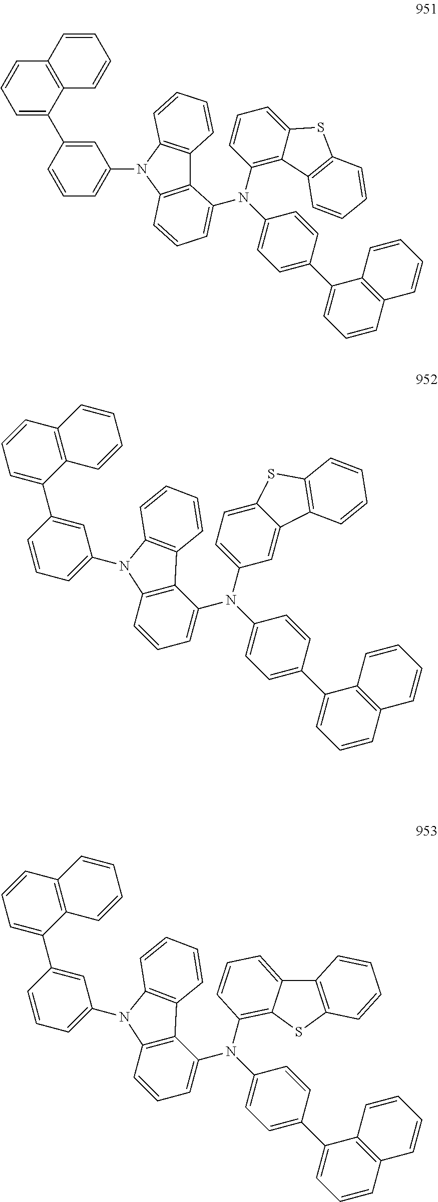 [SMILES: c1cc(-c2cccc3ccccc23)cc(-n2c3ccccc3c3c(N(c4ccc(-c5cccc6ccccc56)cc4)c4cccc5sc6ccccc6c45)cccc32)c1 ; c1cc(-c2cccc3ccccc23)cc(-n2c3ccccc3c3c(N(c4ccc(-c5cccc6ccccc56)cc4)c4ccc5sc6ccccc6c5c4)cccc32)c1 ; c1cc(-c2cccc3ccccc23)cc(-n2c3ccccc3c3c(N(c4ccc(-c5cccc6ccccc56)cc4)c4cccc5c4sc4ccccc45)cccc32)c1] 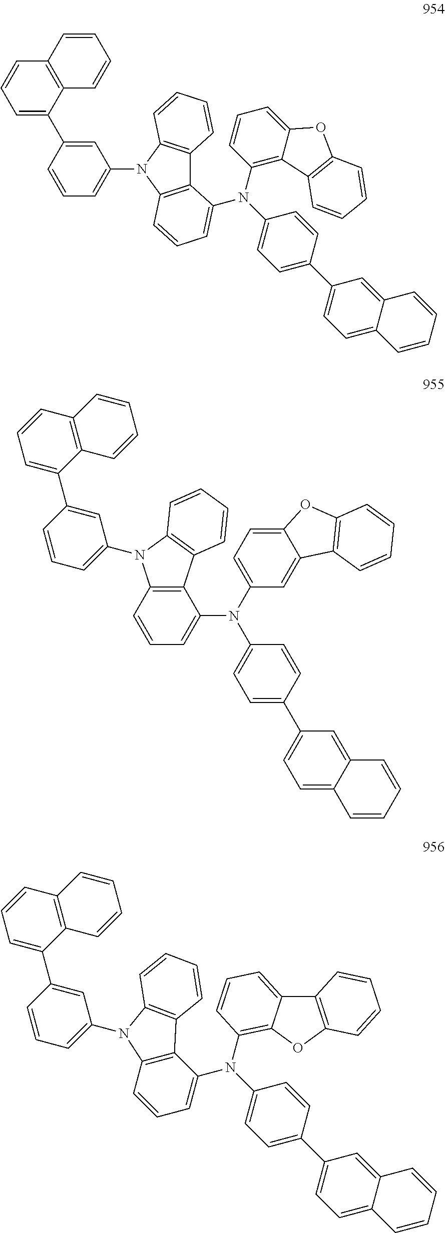 [SMILES: c1cc(-c2cccc3ccccc23)cc(-n2c3ccccc3c3c(N(c4ccc(-c5ccc6ccccc6c5)cc4)c4cccc5oc6ccccc6c45)cccc32)c1 ; c1cc(-c2cccc3ccccc23)cc(-n2c3ccccc3c3c(N(c4ccc(-c5ccc6ccccc6c5)cc4)c4ccc5oc6ccccc6c5c4)cccc32)c1 ; c1cc(-c2cccc3ccccc23)cc(-n2c3ccccc3c3c(N(c4ccc(-c5ccc6ccccc6c5)cc4)c4cccc5c4oc4ccccc45)cccc32)c1] 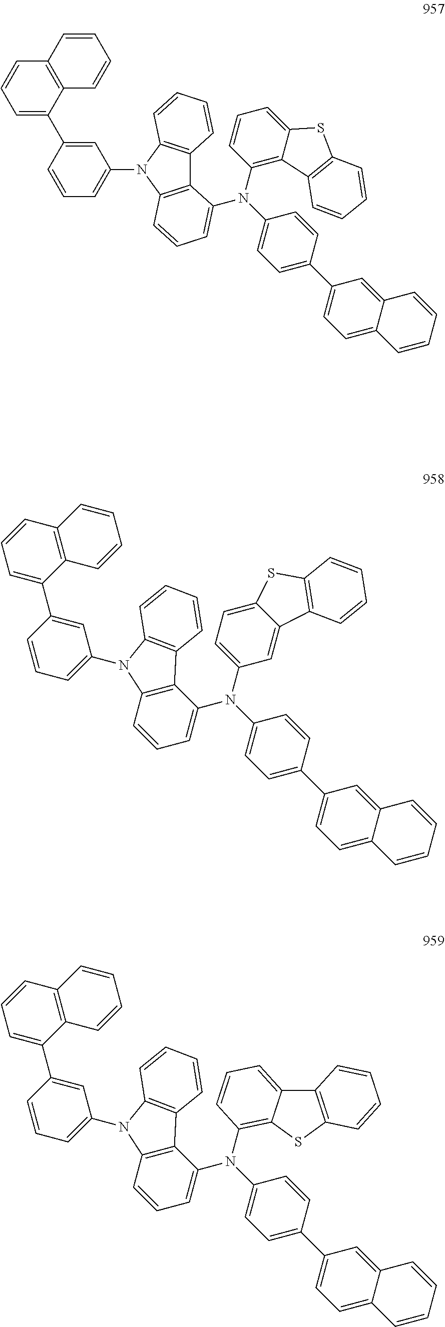 [SMILES: c1cc(-c2cccc3ccccc23)cc(-n2c3ccccc3c3c(N(c4ccc(-c5ccc6ccccc6c5)cc4)c4cccc5sc6ccccc6c45)cccc32)c1 ; c1cc(-c2cccc3ccccc23)cc(-n2c3ccccc3c3c(N(c4ccc(-c5ccc6ccccc6c5)cc4)c4ccc5sc6ccccc6c5c4)cccc32)c1 ; c1cc(-c2cccc3ccccc23)cc(-n2c3ccccc3c3c(N(c4ccc(-c5ccc6ccccc6c5)cc4)c4cccc5c4sc4ccccc45)cccc32)c1] 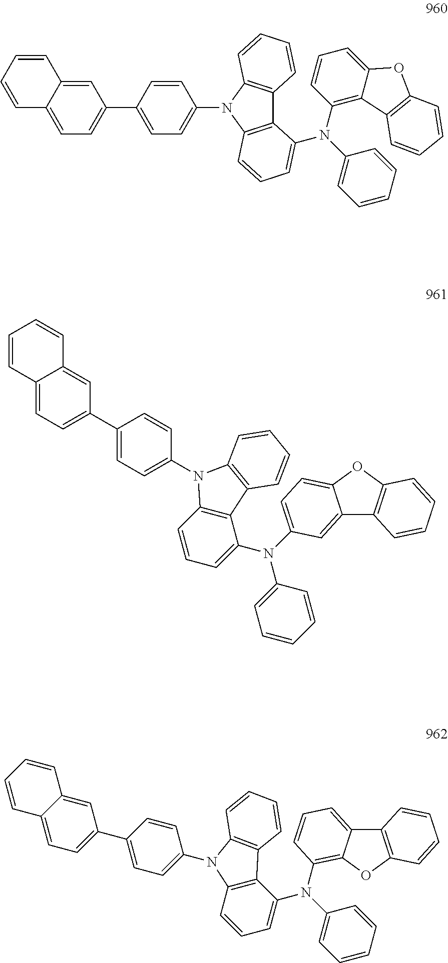 [SMILES: c1ccc(N(c2cccc3oc4ccccc4c23)c2cccc3c2c2ccccc2n3-c2ccc(-c3ccc4ccccc4c3)cc2)cc1 ; c1ccc(N(c2ccc3oc4ccccc4c3c2)c2cccc3c2c2ccccc2n3-c2ccc(-c3ccc4ccccc4c3)cc2)cc1 ; c1ccc(N(c2cccc3c2oc2ccccc23)c2cccc3c2c2ccccc2n3-c2ccc(-c3ccc4ccccc4c3)cc2)cc1] 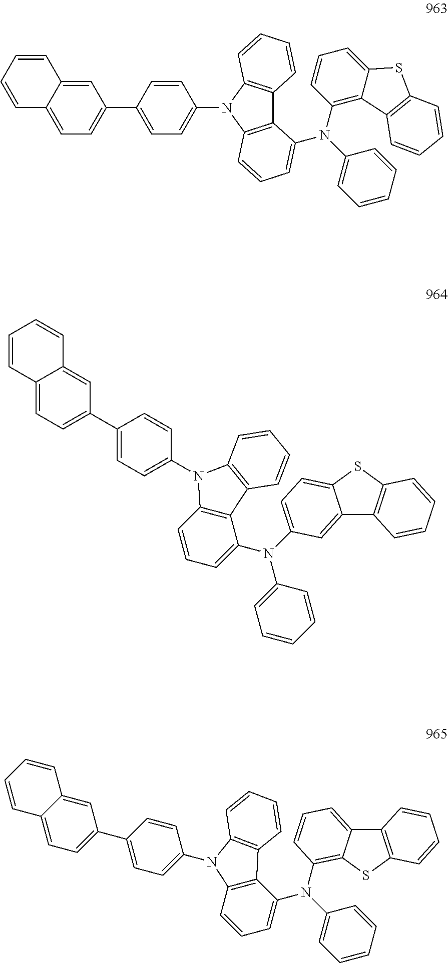 [SMILES: c1ccc(N(c2cccc3sc4ccccc4c23)c2cccc3c2c2ccccc2n3-c2ccc(-c3ccc4ccccc4c3)cc2)cc1 ; c1ccc(N(c2ccc3sc4ccccc4c3c2)c2cccc3c2c2ccccc2n3-c2ccc(-c3ccc4ccccc4c3)cc2)cc1 ; c1ccc(N(c2cccc3c2sc2ccccc23)c2cccc3c2c2ccccc2n3-c2ccc(-c3ccc4ccccc4c3)cc2)cc1] 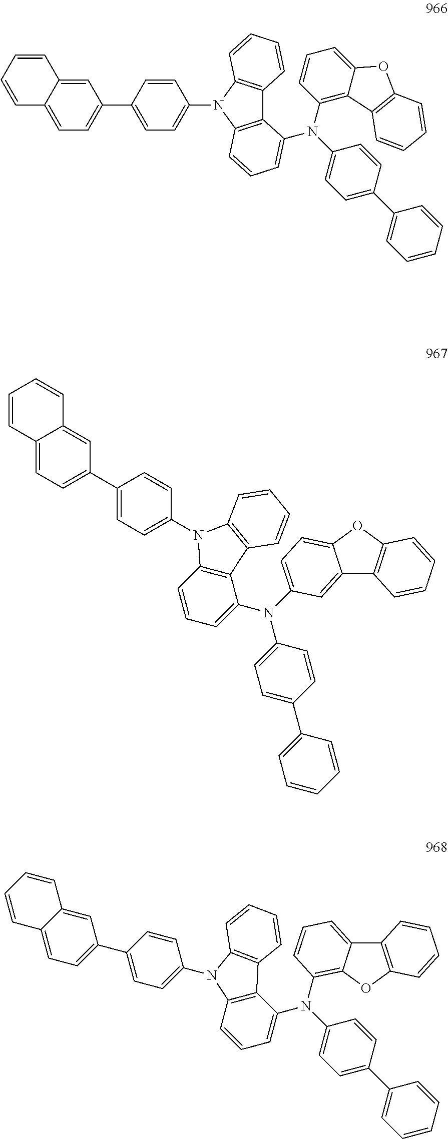 [SMILES: c1ccc(-c2ccc(N(c3cccc4oc5ccccc5c34)c3cccc4c3c3ccccc3n4-c3ccc(-c4ccc5ccccc5c4)cc3)cc2)cc1 ; c1ccc(-c2ccc(N(c3ccc4oc5ccccc5c4c3)c3cccc4c3c3ccccc3n4-c3ccc(-c4ccc5ccccc5c4)cc3)cc2)cc1 ; c1ccc(-c2ccc(N(c3cccc4c3oc3ccccc34)c3cccc4c3c3ccccc3n4-c3ccc(-c4ccc5ccccc5c4)cc3)cc2)cc1] 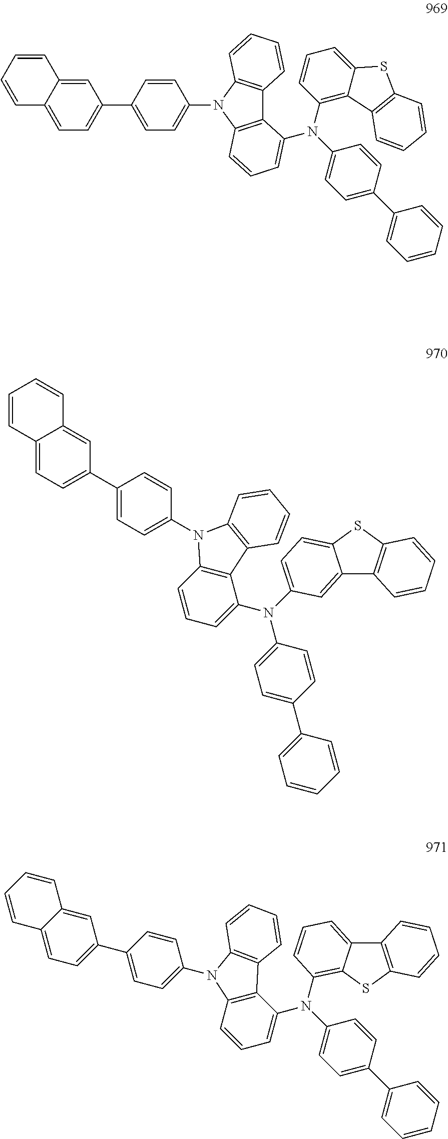 [SMILES: c1ccc(-c2ccc(N(c3cccc4sc5ccccc5c34)c3cccc4c3c3ccccc3n4-c3ccc(-c4ccc5ccccc5c4)cc3)cc2)cc1 ; c1ccc(-c2ccc(N(c3ccc4sc5ccccc5c4c3)c3cccc4c3c3ccccc3n4-c3ccc(-c4ccc5ccccc5c4)cc3)cc2)cc1 ; c1ccc(-c2ccc(N(c3cccc4c3sc3ccccc34)c3cccc4c3c3ccccc3n4-c3ccc(-c4ccc5ccccc5c4)cc3)cc2)cc1] 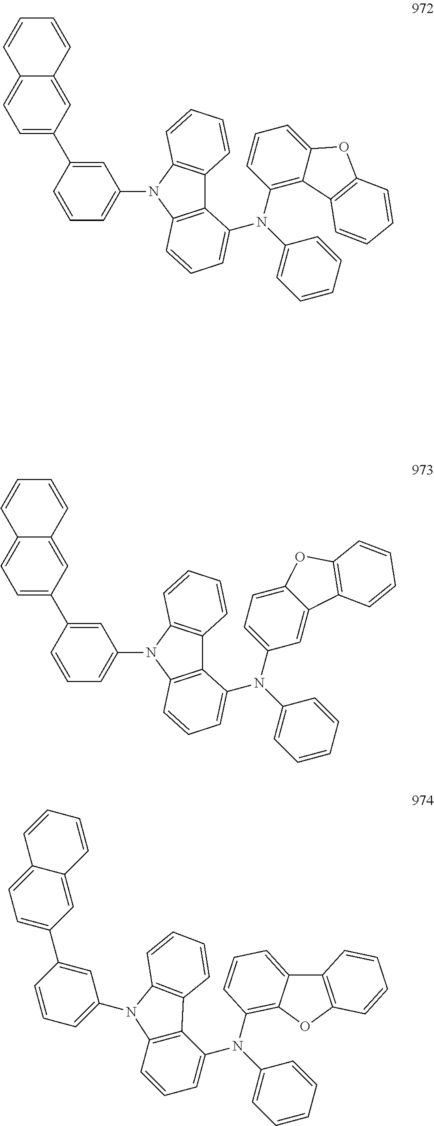 [SMILES: c1ccc(N(c2cccc3oc4ccccc4c23)c2cccc3c2c2ccccc2n3-c2cccc(-c3ccc4ccccc4c3)c2)cc1 ; c1ccc(N(c2ccc3oc4ccccc4c3c2)c2cccc3c2c2ccccc2n3-c2cccc(-c3ccc4ccccc4c3)c2)cc1 ; c1ccc(N(c2cccc3c2oc2ccccc23)c2cccc3c2c2ccccc2n3-c2cccc(-c3ccc4ccccc4c3)c2)cc1] 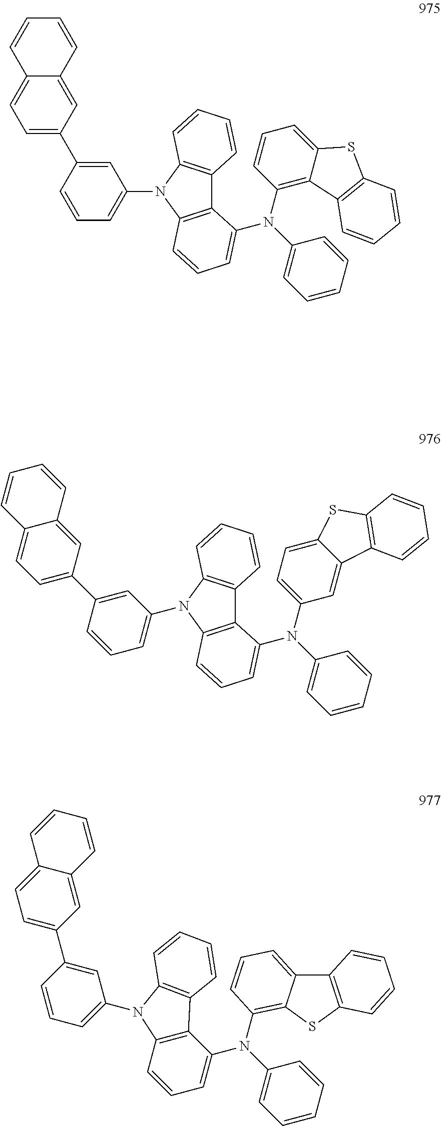 [SMILES: c1ccc(N(c2cccc3sc4ccccc4c23)c2cccc3c2c2ccccc2n3-c2cccc(-c3ccc4ccccc4c3)c2)cc1 ; c1ccc(N(c2ccc3sc4ccccc4c3c2)c2cccc3c2c2ccccc2n3-c2cccc(-c3ccc4ccccc4c3)c2)cc1 ; c1ccc(N(c2cccc3c2sc2ccccc23)c2cccc3c2c2ccccc2n3-c2cccc(-c3ccc4ccccc4c3)c2)cc1] 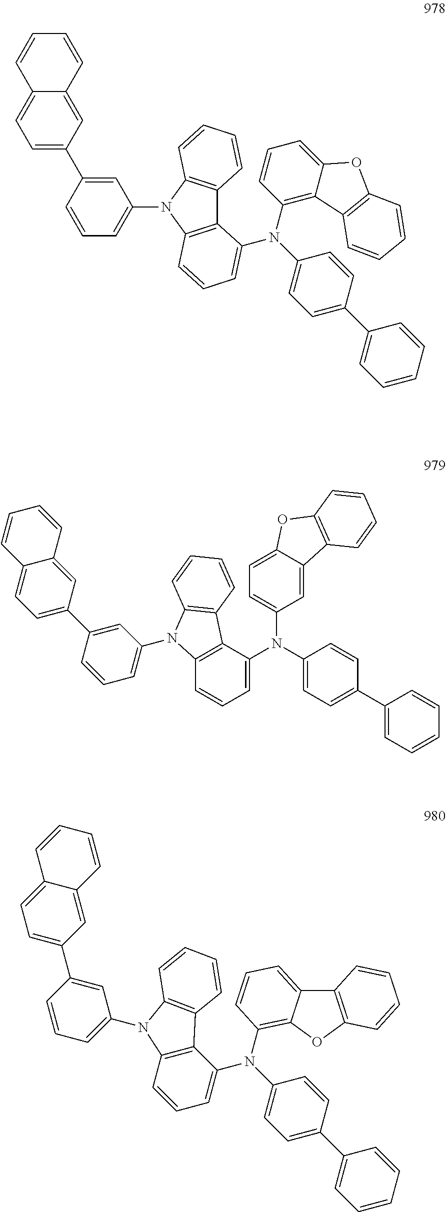 [SMILES: c1ccc(-c2ccc(N(c3cccc4oc5ccccc5c34)c3cccc4c3c3ccccc3n4-c3cccc(-c4ccc5ccccc5c4)c3)cc2)cc1 ; c1ccc(-c2ccc(N(c3ccc4oc5ccccc5c4c3)c3cccc4c3c3ccccc3n4-c3cccc(-c4ccc5ccccc5c4)c3)cc2)cc1 ; c1ccc(-c2ccc(N(c3cccc4c3oc3ccccc34)c3cccc4c3c3ccccc3n4-c3cccc(-c4ccc5ccccc5c4)c3)cc2)cc1] 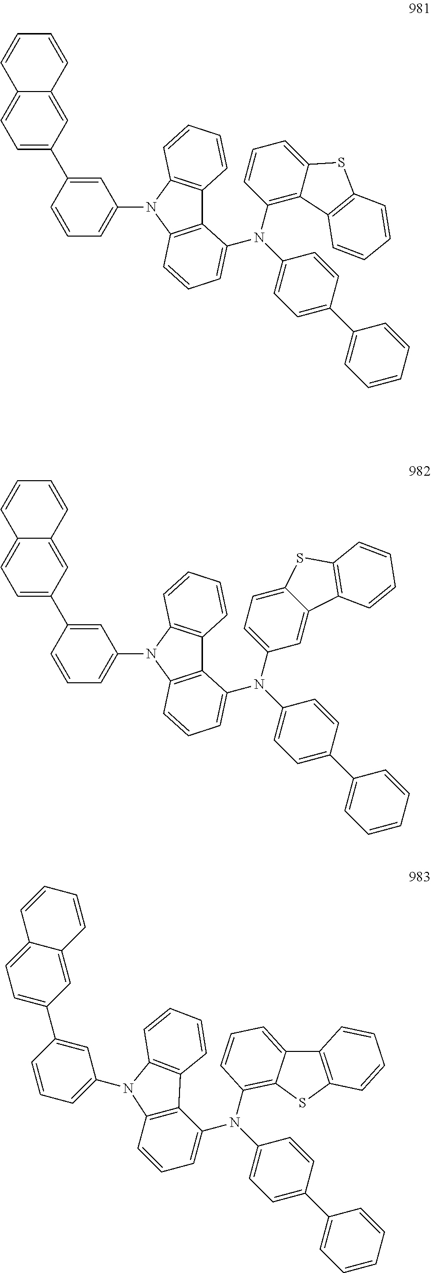 [SMILES: c1ccc(-c2ccc(N(c3cccc4sc5ccccc5c34)c3cccc4c3c3ccccc3n4-c3cccc(-c4ccc5ccccc5c4)c3)cc2)cc1 ; c1ccc(-c2ccc(N(c3ccc4sc5ccccc5c4c3)c3cccc4c3c3ccccc3n4-c3cccc(-c4ccc5ccccc5c4)c3)cc2)cc1 ; c1ccc(-c2ccc(N(c3cccc4c3sc3ccccc34)c3cccc4c3c3ccccc3n4-c3cccc(-c4ccc5ccccc5c4)c3)cc2)cc1] 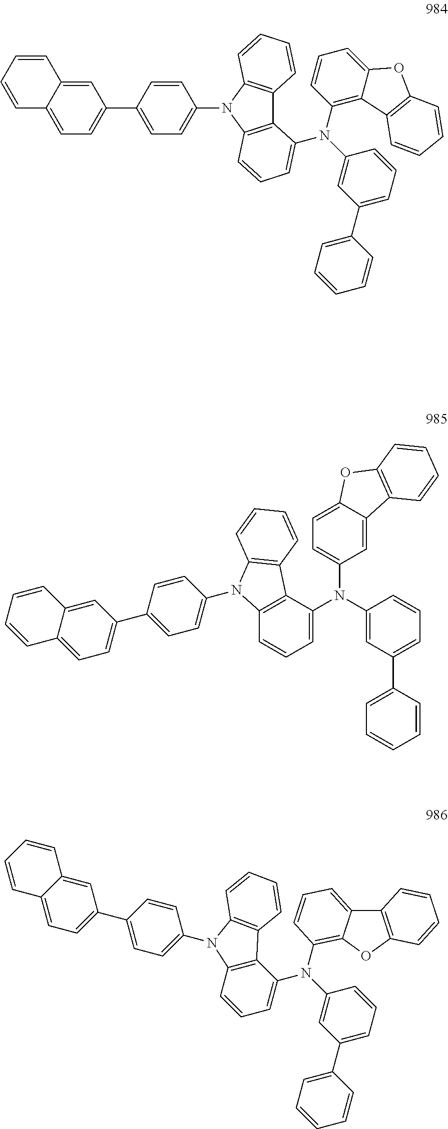 [SMILES: c1ccc(-c2cccc(N(c3cccc4oc5ccccc5c34)c3cccc4c3c3ccccc3n4-c3ccc(-c4ccc5ccccc5c4)cc3)c2)cc1 ; c1ccc(-c2cccc(N(c3ccc4oc5ccccc5c4c3)c3cccc4c3c3ccccc3n4-c3ccc(-c4ccc5ccccc5c4)cc3)c2)cc1 ; c1ccc(-c2cccc(N(c3cccc4c3oc3ccccc34)c3cccc4c3c3ccccc3n4-c3ccc(-c4ccc5ccccc5c4)cc3)c2)cc1] 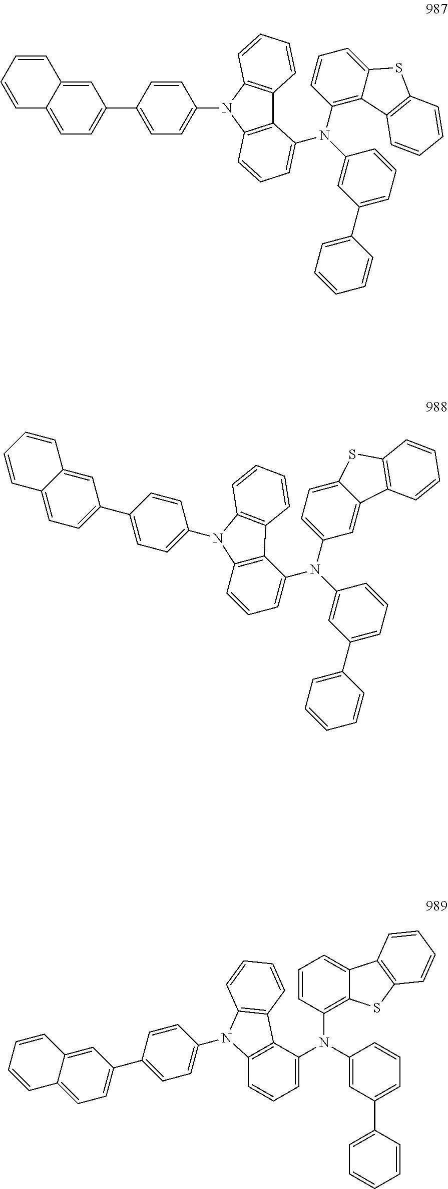 [SMILES: c1ccc(-c2cccc(N(c3cccc4sc5ccccc5c34)c3cccc4c3c3ccccc3n4-c3ccc(-c4ccc5ccccc5c4)cc3)c2)cc1 ; c1ccc(-c2cccc(N(c3ccc4sc5ccccc5c4c3)c3cccc4c3c3ccccc3n4-c3ccc(-c4ccc5ccccc5c4)cc3)c2)cc1 ; c1ccc(-c2cccc(N(c3cccc4c3sc3ccccc34)c3cccc4c3c3ccccc3n4-c3ccc(-c4ccc5ccccc5c4)cc3)c2)cc1] 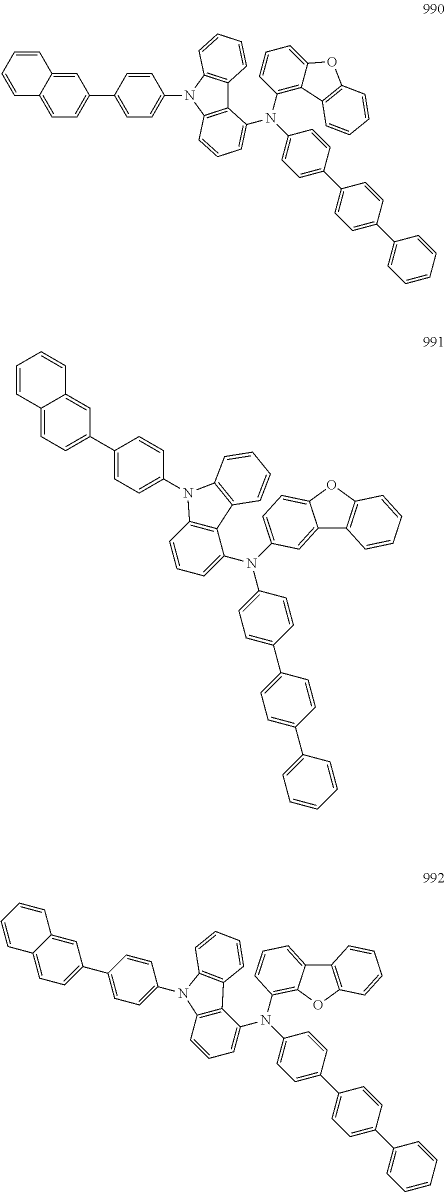 [SMILES: c1ccc(-c2ccc(-c3ccc(N(c4cccc5oc6ccccc6c45)c4cccc5c4c4ccccc4n5-c4ccc(-c5ccc6ccccc6c5)cc4)cc3)cc2)cc1 ; c1ccc(-c2ccc(-c3ccc(N(c4ccc5oc6ccccc6c5c4)c4cccc5c4c4ccccc4n5-c4ccc(-c5ccc6ccccc6c5)cc4)cc3)cc2)cc1 ; c1ccc(-c2ccc(-c3ccc(N(c4cccc5c4oc4ccccc45)c4cccc5c4c4ccccc4n5-c4ccc(-c5ccc6ccccc6c5)cc4)cc3)cc2)cc1] 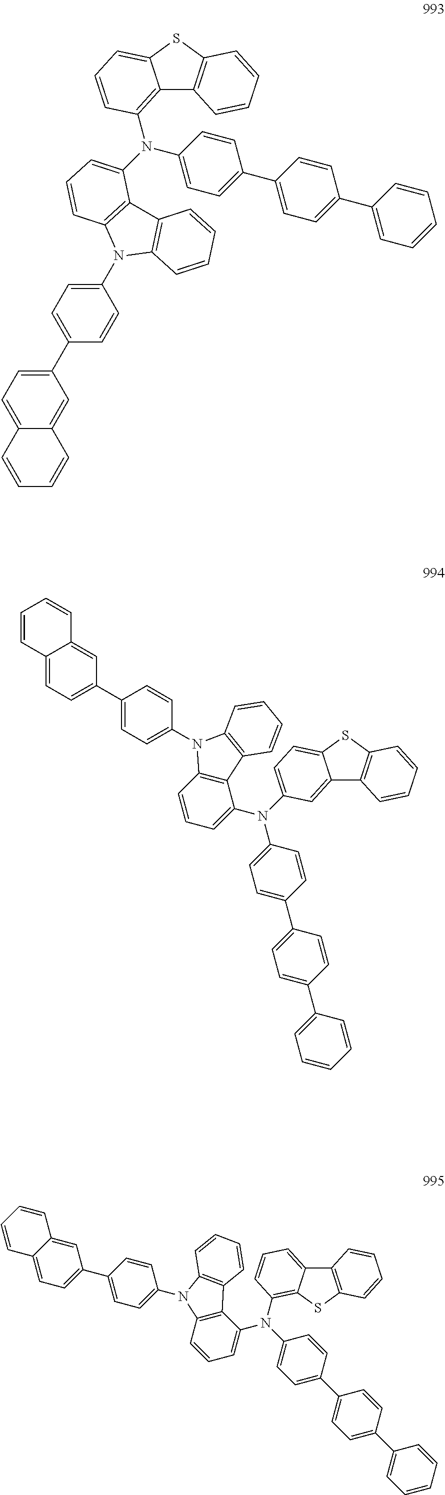 [SMILES: c1ccc(-c2ccc(-c3ccc(N(c4cccc5sc6ccccc6c45)c4cccc5c4c4ccccc4n5-c4ccc(-c5ccc6ccccc6c5)cc4)cc3)cc2)cc1 ; c1ccc(-c2ccc(-c3ccc(N(c4ccc5sc6ccccc6c5c4)c4cccc5c4c4ccccc4n5-c4ccc(-c5ccc6ccccc6c5)cc4)cc3)cc2)cc1 ; c1ccc(-c2ccc(-c3ccc(N(c4cccc5c4sc4ccccc45)c4cccc5c4c4ccccc4n5-c4ccc(-c5ccc6ccccc6c5)cc4)cc3)cc2)cc1] 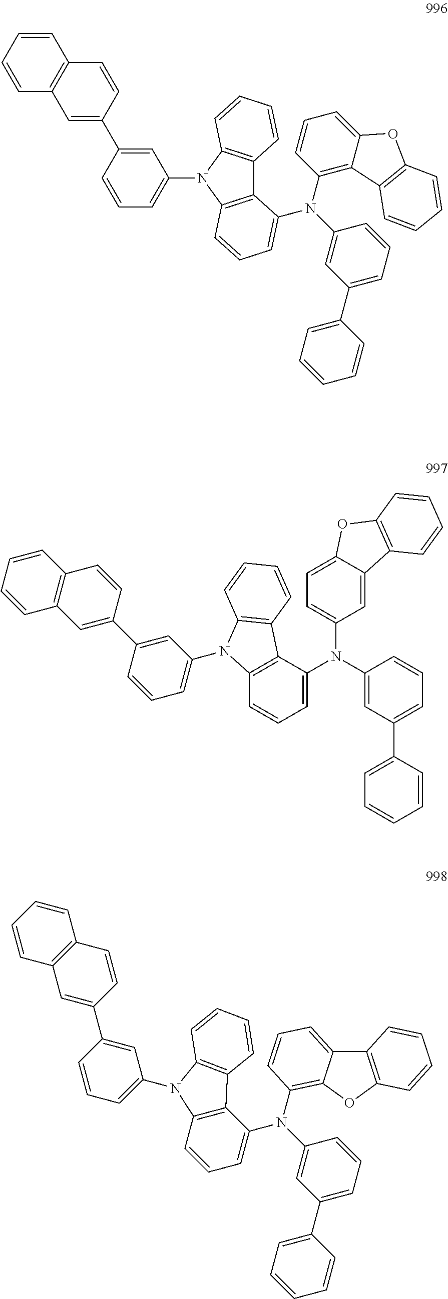 [SMILES: c1ccc(-c2cccc(N(c3cccc4oc5ccccc5c34)c3cccc4c3c3ccccc3n4-c3cccc(-c4ccc5ccccc5c4)c3)c2)cc1 ; c1ccc(-c2cccc(N(c3ccc4oc5ccccc5c4c3)c3cccc4c3c3ccccc3n4-c3cccc(-c4ccc5ccccc5c4)c3)c2)cc1 ; c1ccc(-c2cccc(N(c3cccc4c3oc3ccccc34)c3cccc4c3c3ccccc3n4-c3cccc(-c4ccc5ccccc5c4)c3)c2)cc1] 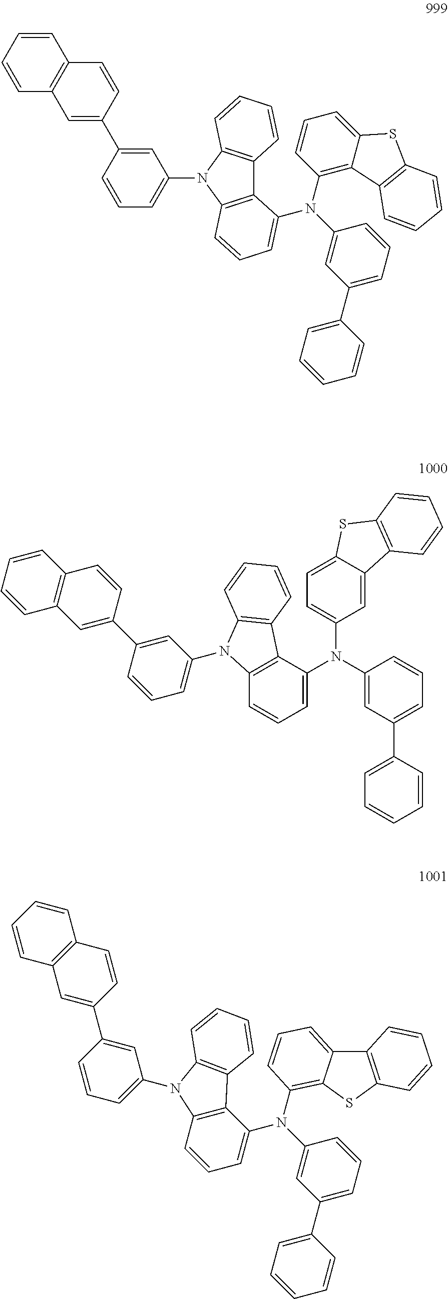 [SMILES: c1ccc(-c2cccc(N(c3cccc4sc5ccccc5c34)c3cccc4c3c3ccccc3n4-c3cccc(-c4ccc5ccccc5c4)c3)c2)cc1 ; c1ccc(-c2cccc(N(c3ccc4sc5ccccc5c4c3)c3cccc4c3c3ccccc3n4-c3cccc(-c4ccc5ccccc5c4)c3)c2)cc1 ; c1ccc(-c2cccc(N(c3cccc4c3sc3ccccc34)c3cccc4c3c3ccccc3n4-c3cccc(-c4ccc5ccccc5c4)c3)c2)cc1] 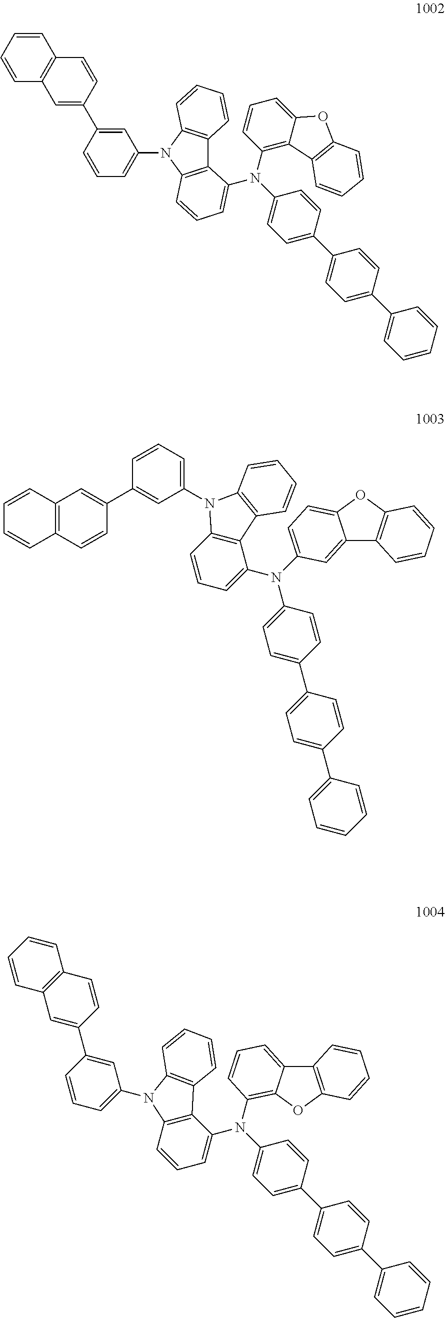 [SMILES: c1ccc(-c2ccc(-c3ccc(N(c4cccc5oc6ccccc6c45)c4cccc5c4c4ccccc4n5-c4cccc(-c5ccc6ccccc6c5)c4)cc3)cc2)cc1 ; c1ccc(-c2ccc(-c3ccc(N(c4ccc5oc6ccccc6c5c4)c4cccc5c4c4ccccc4n5-c4cccc(-c5ccc6ccccc6c5)c4)cc3)cc2)cc1 ; c1ccc(-c2ccc(-c3ccc(N(c4cccc5c4oc4ccccc45)c4cccc5c4c4ccccc4n5-c4cccc(-c5ccc6ccccc6c5)c4)cc3)cc2)cc1] 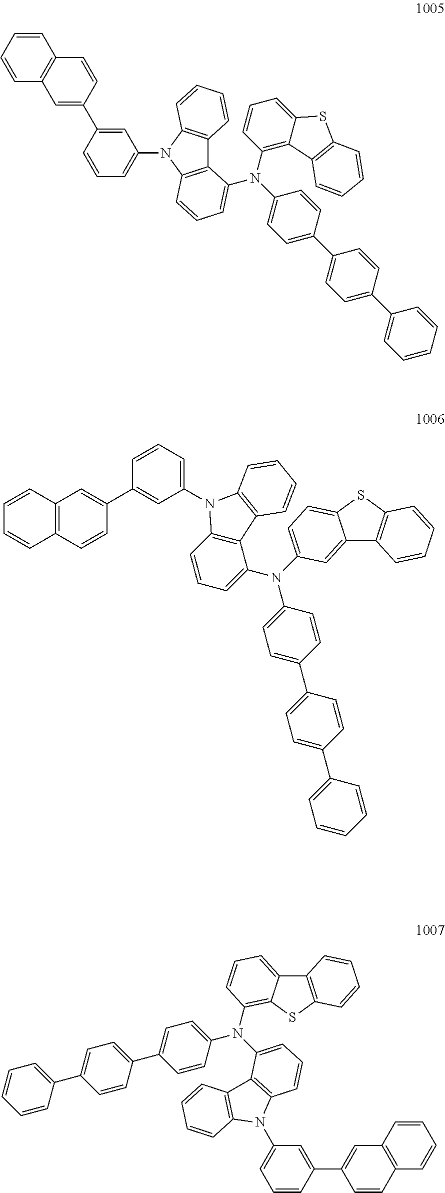 [SMILES: c1ccc(-c2ccc(-c3ccc(N(c4cccc5sc6ccccc6c45)c4cccc5c4c4ccccc4n5-c4cccc(-c5ccc6ccccc6c5)c4)cc3)cc2)cc1 ; c1ccc(-c2ccc(-c3ccc(N(c4ccc5sc6ccccc6c5c4)c4cccc5c4c4ccccc4n5-c4cccc(-c5ccc6ccccc6c5)c4)cc3)cc2)cc1 ; c1ccc(-c2ccc(-c3ccc(N(c4cccc5c4sc4ccccc45)c4cccc5c4c4ccccc4n5-c4cccc(-c5ccc6ccccc6c5)c4)cc3)cc2)cc1] 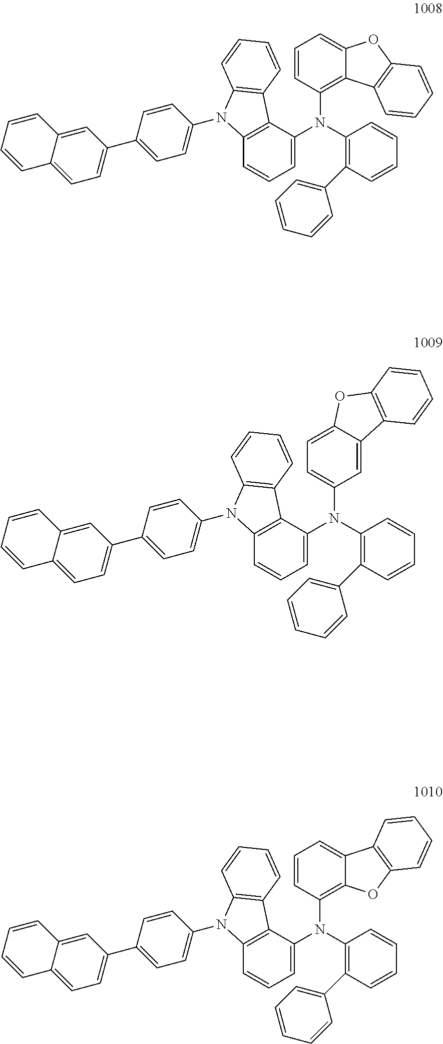 [SMILES: c1ccc(-c2ccccc2N(c2cccc3oc4ccccc4c23)c2cccc3c2c2ccccc2n3-c2ccc(-c3ccc4ccccc4c3)cc2)cc1 ; c1ccc(-c2ccccc2N(c2ccc3oc4ccccc4c3c2)c2cccc3c2c2ccccc2n3-c2ccc(-c3ccc4ccccc4c3)cc2)cc1 ; c1ccc(-c2ccccc2N(c2cccc3c2oc2ccccc23)c2cccc3c2c2ccccc2n3-c2ccc(-c3ccc4ccccc4c3)cc2)cc1] 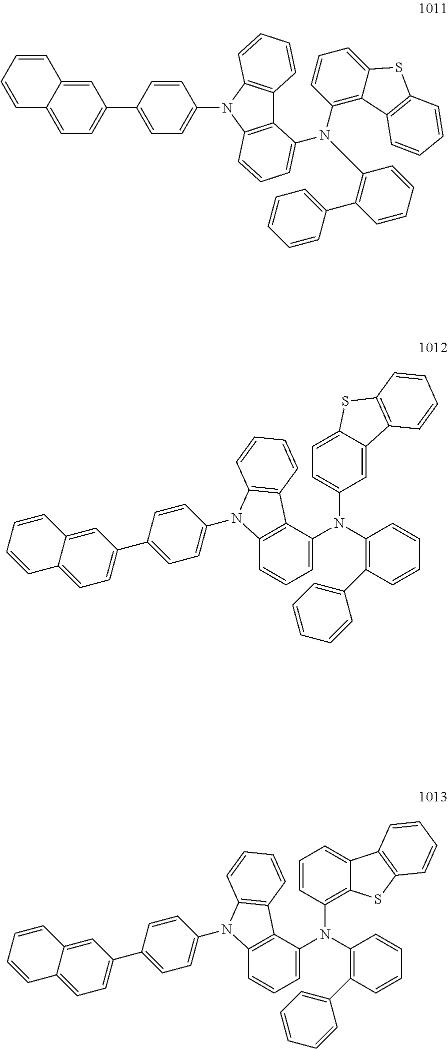 [SMILES: c1ccc(-c2ccccc2N(c2cccc3sc4ccccc4c23)c2cccc3c2c2ccccc2n3-c2ccc(-c3ccc4ccccc4c3)cc2)cc1 ; c1ccc(-c2ccccc2N(c2ccc3sc4ccccc4c3c2)c2cccc3c2c2ccccc2n3-c2ccc(-c3ccc4ccccc4c3)cc2)cc1 ; c1ccc(-c2ccccc2N(c2cccc3c2sc2ccccc23)c2cccc3c2c2ccccc2n3-c2ccc(-c3ccc4ccccc4c3)cc2)cc1] 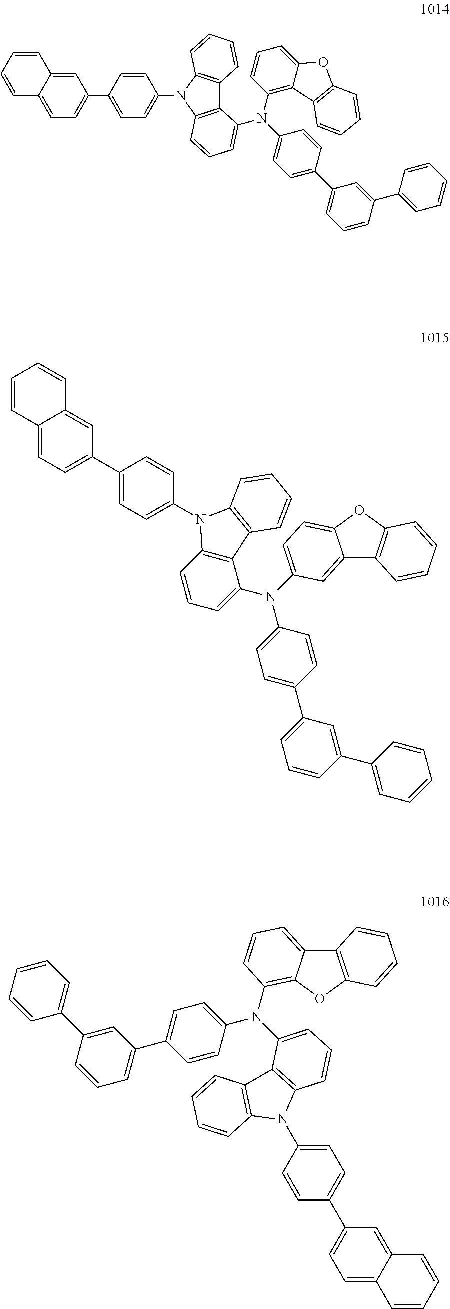 [SMILES: c1ccc(-c2cccc(-c3ccc(N(c4cccc5oc6ccccc6c45)c4cccc5c4c4ccccc4n5-c4ccc(-c5ccc6ccccc6c5)cc4)cc3)c2)cc1 ; c1ccc(-c2cccc(-c3ccc(N(c4ccc5oc6ccccc6c5c4)c4cccc5c4c4ccccc4n5-c4ccc(-c5ccc6ccccc6c5)cc4)cc3)c2)cc1 ; c1ccc(-c2cccc(-c3ccc(N(c4cccc5c4oc4ccccc45)c4cccc5c4c4ccccc4n5-c4ccc(-c5ccc6ccccc6c5)cc4)cc3)c2)cc1] 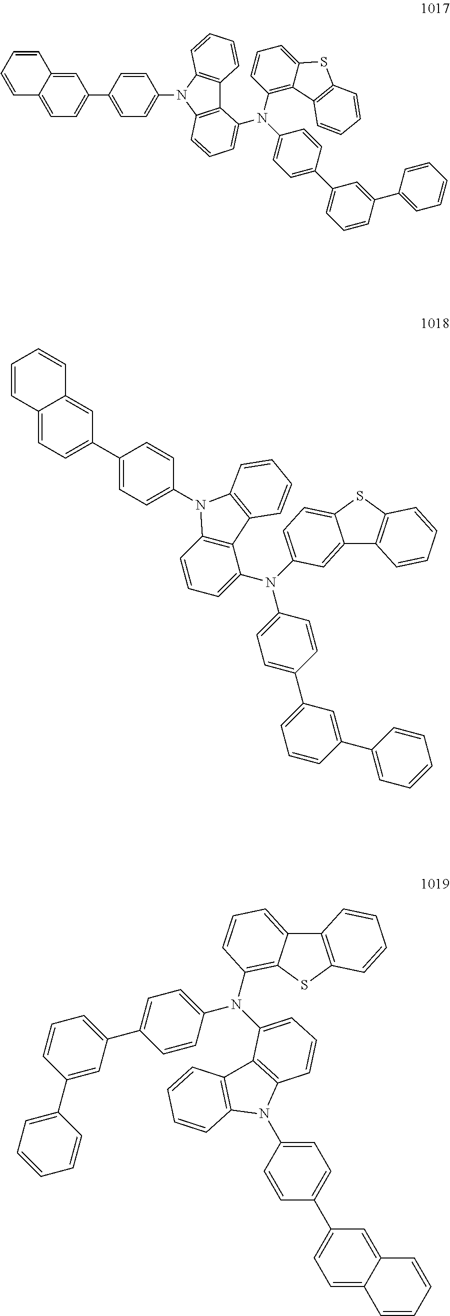 [SMILES: c1ccc(-c2cccc(-c3ccc(N(c4cccc5sc6ccccc6c45)c4cccc5c4c4ccccc4n5-c4ccc(-c5ccc6ccccc6c5)cc4)cc3)c2)cc1 ; c1ccc(-c2cccc(-c3ccc(N(c4ccc5sc6ccccc6c5c4)c4cccc5c4c4ccccc4n5-c4ccc(-c5ccc6ccccc6c5)cc4)cc3)c2)cc1 ; c1ccc(-c2cccc(-c3ccc(N(c4cccc5c4sc4ccccc45)c4cccc5c4c4ccccc4n5-c4ccc(-c5ccc6ccccc6c5)cc4)cc3)c2)cc1] 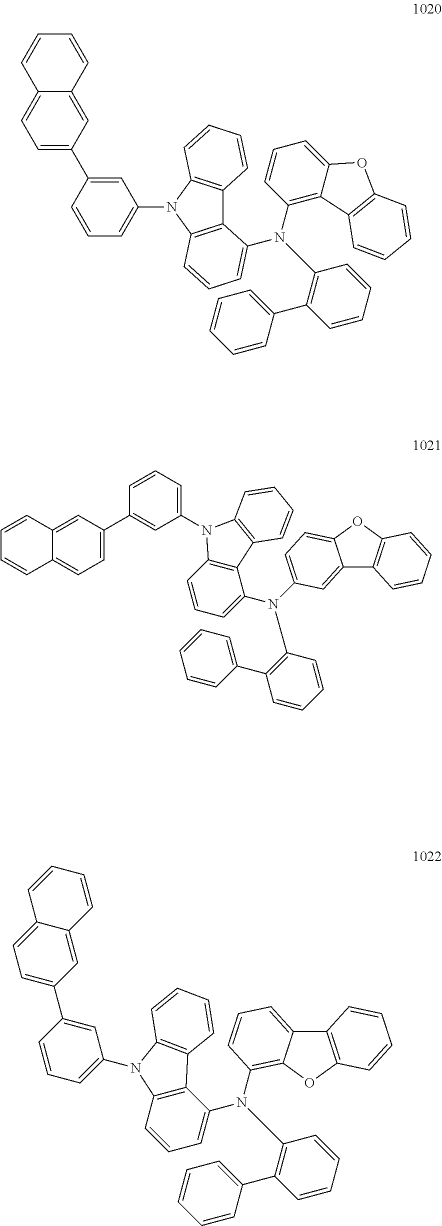 [SMILES: c1ccc(-c2ccccc2N(c2cccc3oc4ccccc4c23)c2cccc3c2c2ccccc2n3-c2cccc(-c3ccc4ccccc4c3)c2)cc1 ; c1ccc(-c2ccccc2N(c2ccc3oc4ccccc4c3c2)c2cccc3c2c2ccccc2n3-c2cccc(-c3ccc4ccccc4c3)c2)cc1 ; c1ccc(-c2ccccc2N(c2cccc3c2oc2ccccc23)c2cccc3c2c2ccccc2n3-c2cccc(-c3ccc4ccccc4c3)c2)cc1] 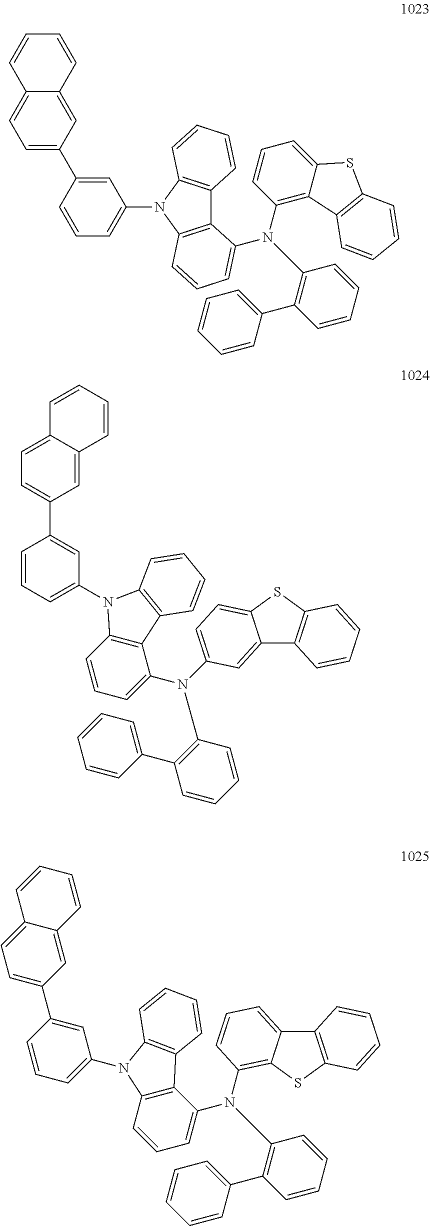 [SMILES: c1ccc(-c2ccccc2N(c2cccc3sc4ccccc4c23)c2cccc3c2c2ccccc2n3-c2cccc(-c3ccc4ccccc4c3)c2)cc1 ; c1ccc(-c2ccccc2N(c2ccc3sc4ccccc4c3c2)c2cccc3c2c2ccccc2n3-c2cccc(-c3ccc4ccccc4c3)c2)cc1 ; c1ccc(-c2ccccc2N(c2cccc3c2sc2ccccc23)c2cccc3c2c2ccccc2n3-c2cccc(-c3ccc4ccccc4c3)c2)cc1] 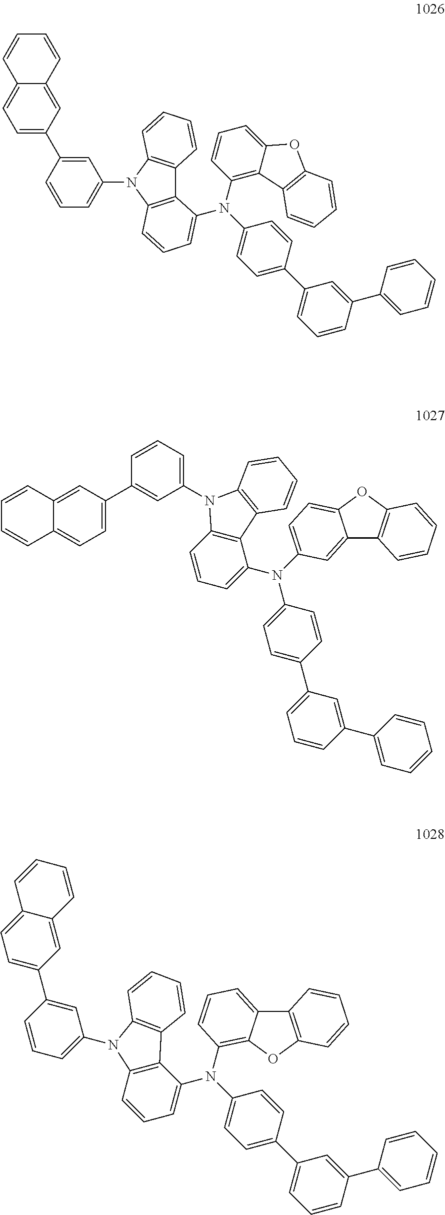 [SMILES: c1ccc(-c2cccc(-c3ccc(N(c4cccc5oc6ccccc6c45)c4cccc5c4c4ccccc4n5-c4cccc(-c5ccc6ccccc6c5)c4)cc3)c2)cc1 ; c1ccc(-c2cccc(-c3ccc(N(c4ccc5oc6ccccc6c5c4)c4cccc5c4c4ccccc4n5-c4cccc(-c5ccc6ccccc6c5)c4)cc3)c2)cc1 ; c1ccc(-c2cccc(-c3ccc(N(c4cccc5c4oc4ccccc45)c4cccc5c4c4ccccc4n5-c4cccc(-c5ccc6ccccc6c5)c4)cc3)c2)cc1] 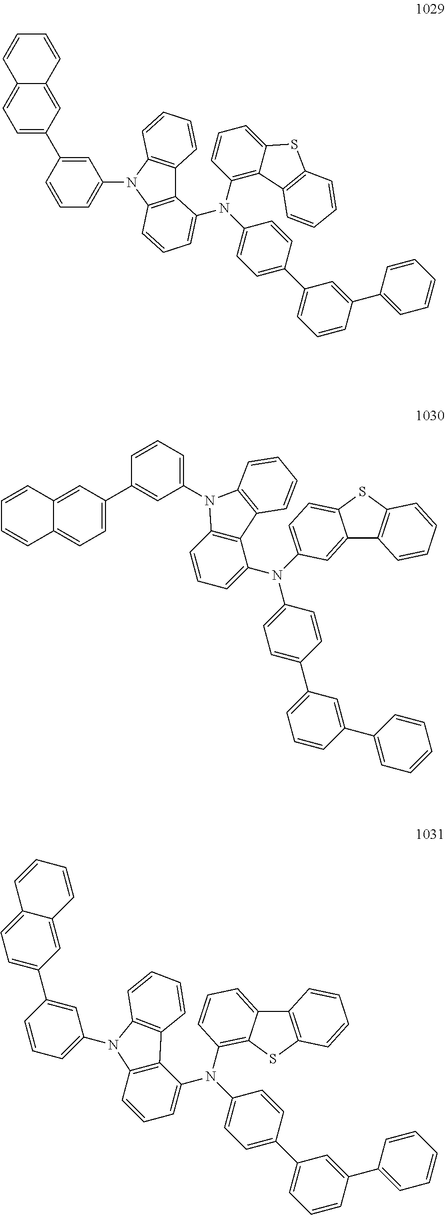 [SMILES: c1ccc(-c2cccc(-c3ccc(N(c4cccc5sc6ccccc6c45)c4cccc5c4c4ccccc4n5-c4cccc(-c5ccc6ccccc6c5)c4)cc3)c2)cc1 ; c1ccc(-c2cccc(-c3ccc(N(c4ccc5sc6ccccc6c5c4)c4cccc5c4c4ccccc4n5-c4cccc(-c5ccc6ccccc6c5)c4)cc3)c2)cc1 ; c1ccc(-c2cccc(-c3ccc(N(c4cccc5c4sc4ccccc45)c4cccc5c4c4ccccc4n5-c4cccc(-c5ccc6ccccc6c5)c4)cc3)c2)cc1] 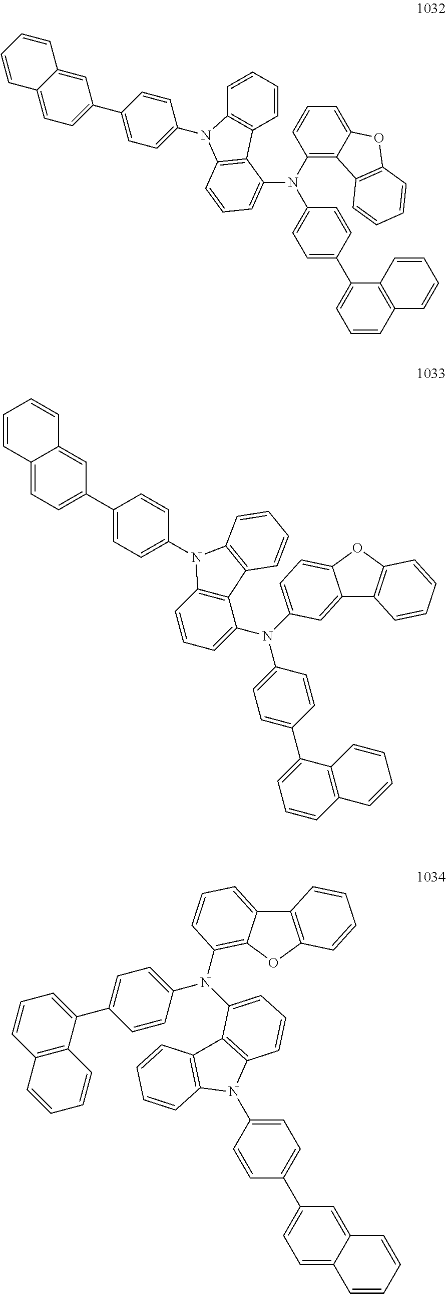 [SMILES: c1ccc2cc(-c3ccc(-n4c5ccccc5c5c(N(c6ccc(-c7cccc8ccccc78)cc6)c6cccc7oc8ccccc8c67)cccc54)cc3)ccc2c1 ; c1ccc2cc(-c3ccc(-n4c5ccccc5c5c(N(c6ccc(-c7cccc8ccccc78)cc6)c6ccc7oc8ccccc8c7c6)cccc54)cc3)ccc2c1 ; c1ccc2cc(-c3ccc(-n4c5ccccc5c5c(N(c6ccc(-c7cccc8ccccc78)cc6)c6cccc7c6oc6ccccc67)cccc54)cc3)ccc2c1] 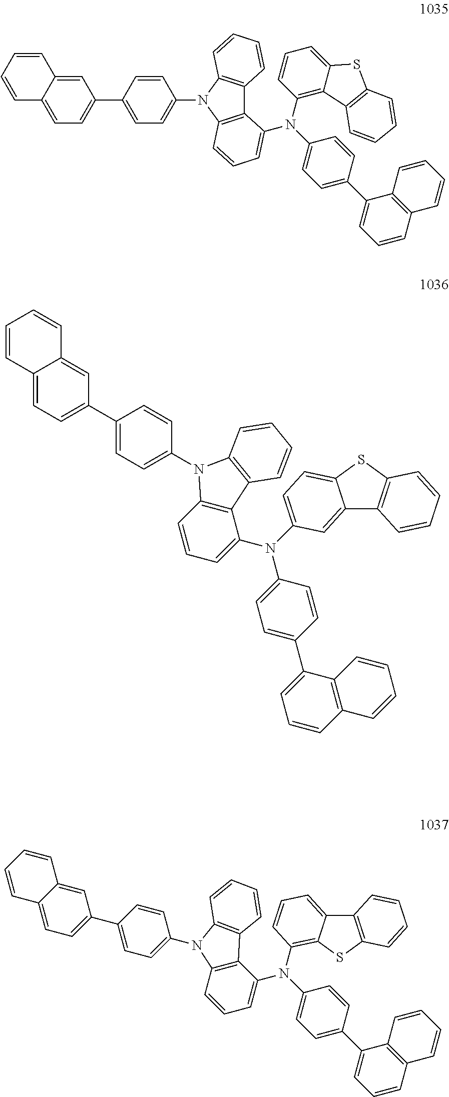 [SMILES: c1ccc2cc(-c3ccc(-n4c5ccccc5c5c(N(c6ccc(-c7cccc8ccccc78)cc6)c6cccc7sc8ccccc8c67)cccc54)cc3)ccc2c1 ; c1ccc2cc(-c3ccc(-n4c5ccccc5c5c(N(c6ccc(-c7cccc8ccccc78)cc6)c6ccc7sc8ccccc8c7c6)cccc54)cc3)ccc2c1 ; c1ccc2cc(-c3ccc(-n4c5ccccc5c5c(N(c6ccc(-c7cccc8ccccc78)cc6)c6cccc7c6sc6ccccc67)cccc54)cc3)ccc2c1] 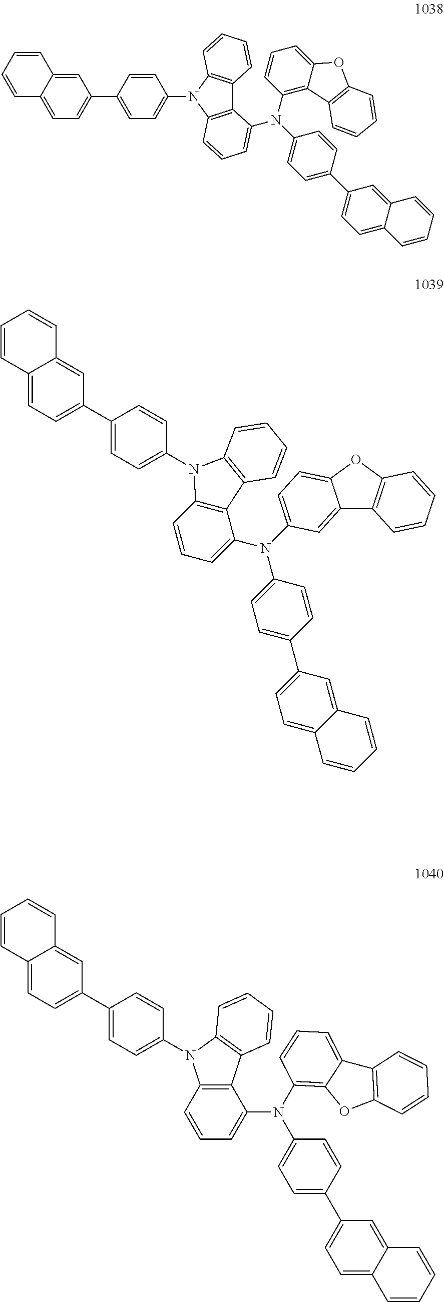 [SMILES: c1ccc2cc(-c3ccc(N(c4cccc5oc6ccccc6c45)c4cccc5c4c4ccccc4n5-c4ccc(-c5ccc6ccccc6c5)cc4)cc3)ccc2c1 ; c1ccc2cc(-c3ccc(N(c4ccc5oc6ccccc6c5c4)c4cccc5c4c4ccccc4n5-c4ccc(-c5ccc6ccccc6c5)cc4)cc3)ccc2c1 ; c1ccc2cc(-c3ccc(N(c4cccc5c4oc4ccccc45)c4cccc5c4c4ccccc4n5-c4ccc(-c5ccc6ccccc6c5)cc4)cc3)ccc2c1] 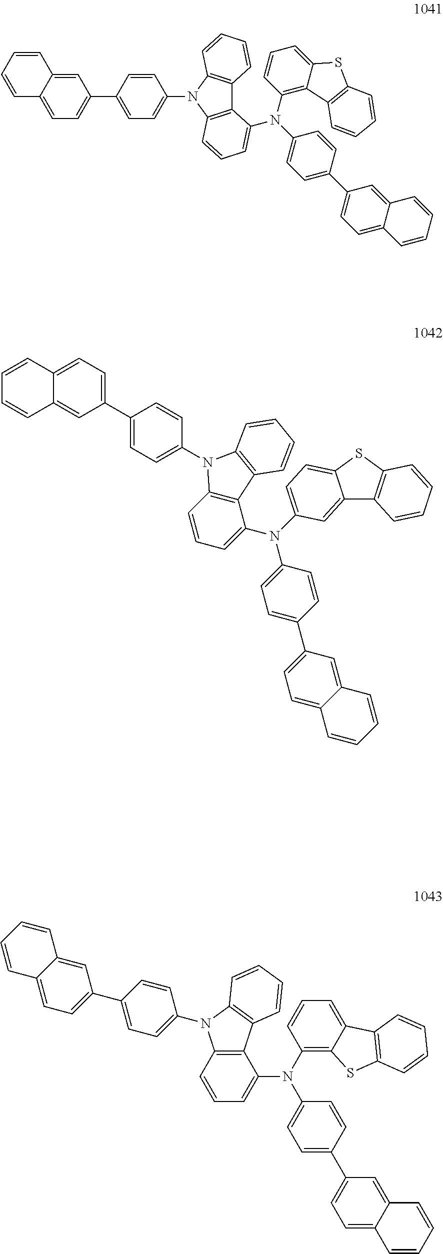 [SMILES: c1ccc2cc(-c3ccc(N(c4cccc5sc6ccccc6c45)c4cccc5c4c4ccccc4n5-c4ccc(-c5ccc6ccccc6c5)cc4)cc3)ccc2c1 ; c1ccc2cc(-c3ccc(N(c4ccc5sc6ccccc6c5c4)c4cccc5c4c4ccccc4n5-c4ccc(-c5ccc6ccccc6c5)cc4)cc3)ccc2c1 ; c1ccc2cc(-c3ccc(N(c4cccc5c4sc4ccccc45)c4cccc5c4c4ccccc4n5-c4ccc(-c5ccc6ccccc6c5)cc4)cc3)ccc2c1] 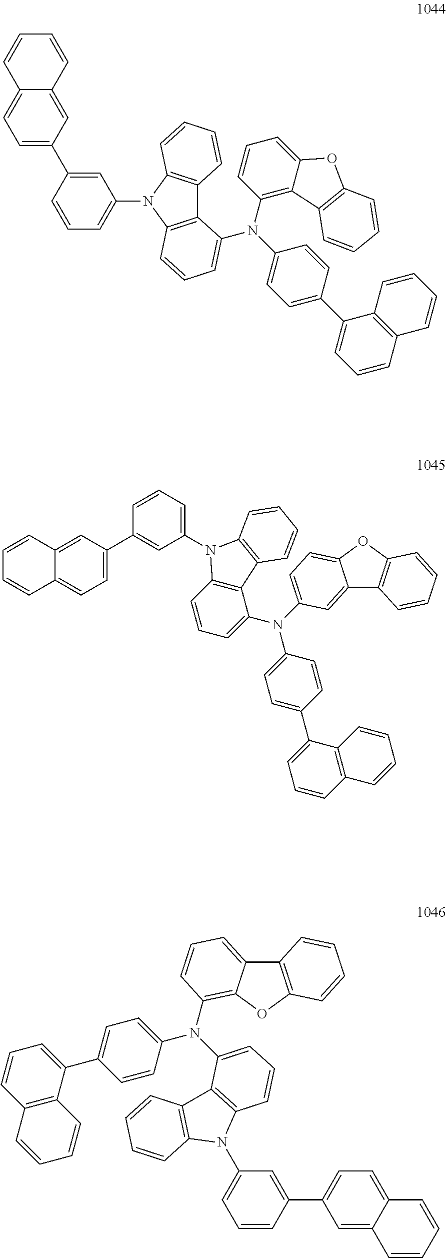 [SMILES: c1cc(-c2ccc3ccccc3c2)cc(-n2c3ccccc3c3c(N(c4ccc(-c5cccc6ccccc56)cc4)c4cccc5oc6ccccc6c45)cccc32)c1 ; c1cc(-c2ccc3ccccc3c2)cc(-n2c3ccccc3c3c(N(c4ccc(-c5cccc6ccccc56)cc4)c4ccc5oc6ccccc6c5c4)cccc32)c1 ; c1cc(-c2ccc3ccccc3c2)cc(-n2c3ccccc3c3c(N(c4ccc(-c5cccc6ccccc56)cc4)c4cccc5c4oc4ccccc45)cccc32)c1] 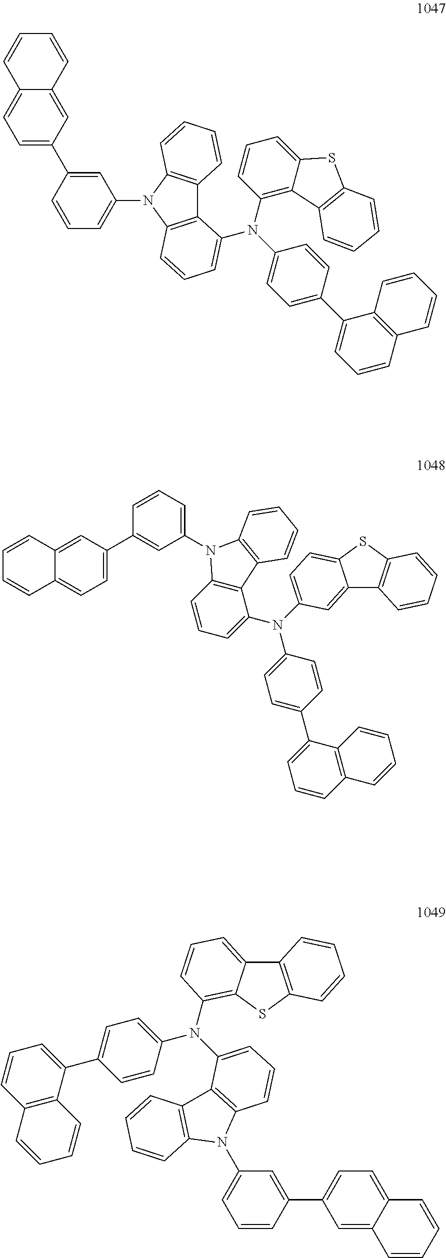 [SMILES: c1cc(-c2ccc3ccccc3c2)cc(-n2c3ccccc3c3c(N(c4ccc(-c5cccc6ccccc56)cc4)c4cccc5sc6ccccc6c45)cccc32)c1 ; c1cc(-c2ccc3ccccc3c2)cc(-n2c3ccccc3c3c(N(c4ccc(-c5cccc6ccccc56)cc4)c4ccc5sc6ccccc6c5c4)cccc32)c1 ; c1cc(-c2ccc3ccccc3c2)cc(-n2c3ccccc3c3c(N(c4ccc(-c5cccc6ccccc56)cc4)c4cccc5c4sc4ccccc45)cccc32)c1] 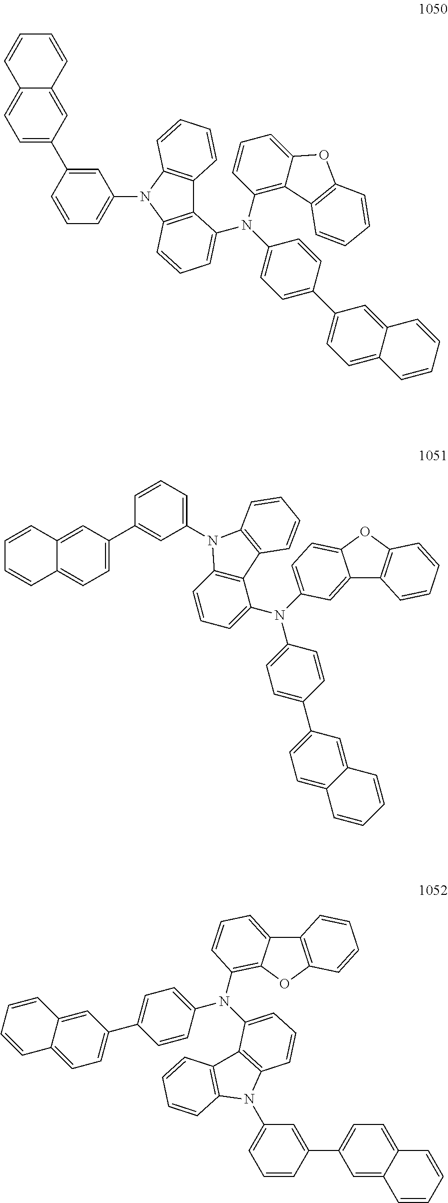 [SMILES: c1cc(-c2ccc3ccccc3c2)cc(-n2c3ccccc3c3c(N(c4ccc(-c5ccc6ccccc6c5)cc4)c4cccc5oc6ccccc6c45)cccc32)c1 ; c1cc(-c2ccc3ccccc3c2)cc(-n2c3ccccc3c3c(N(c4ccc(-c5ccc6ccccc6c5)cc4)c4ccc5oc6ccccc6c5c4)cccc32)c1 ; c1cc(-c2ccc3ccccc3c2)cc(-n2c3ccccc3c3c(N(c4ccc(-c5ccc6ccccc6c5)cc4)c4cccc5c4oc4ccccc45)cccc32)c1] 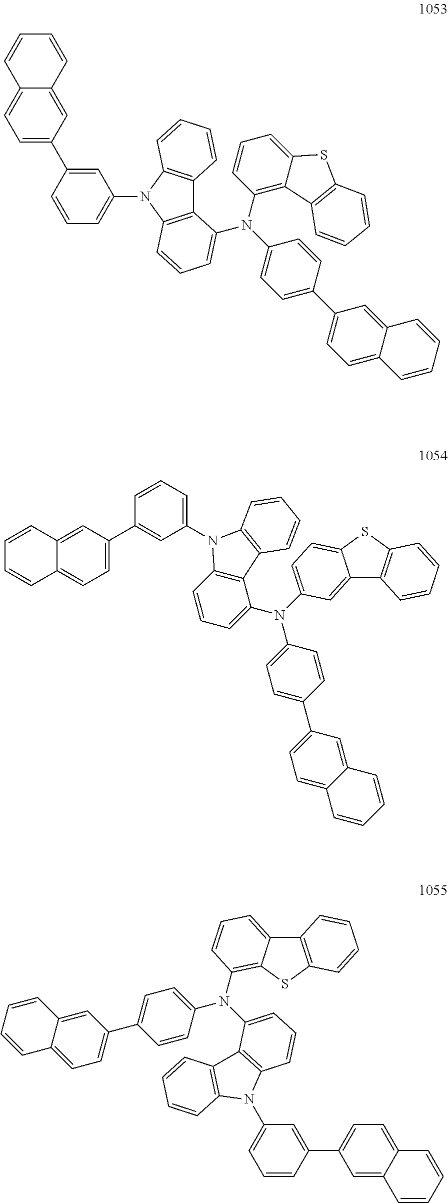 [SMILES: c1cc(-c2ccc3ccccc3c2)cc(-n2c3ccccc3c3c(N(c4ccc(-c5ccc6ccccc6c5)cc4)c4cccc5sc6ccccc6c45)cccc32)c1 ; c1cc(-c2ccc3ccccc3c2)cc(-n2c3ccccc3c3c(N(c4ccc(-c5ccc6ccccc6c5)cc4)c4ccc5sc6ccccc6c5c4)cccc32)c1 ; c1cc(-c2ccc3ccccc3c2)cc(-n2c3ccccc3c3c(N(c4ccc(-c5ccc6ccccc6c5)cc4)c4cccc5c4sc4ccccc45)cccc32)c1] 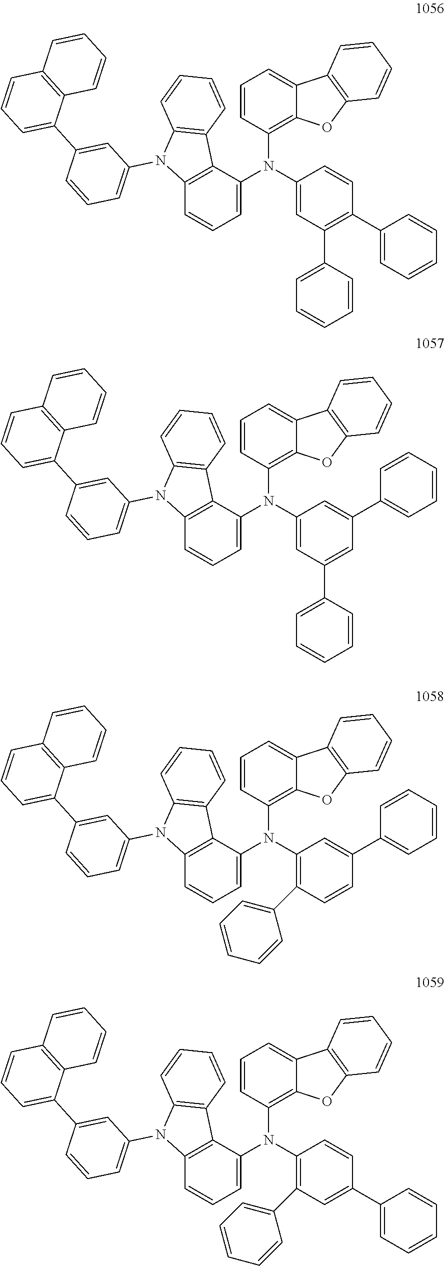 [SMILES: c1ccc(-c2ccc(N(c3cccc4c3oc3ccccc34)c3cccc4c3c3ccccc3n4-c3cccc(-c4cccc5ccccc45)c3)cc2-c2ccccc2)cc1 ; c1ccc(-c2cc(-c3ccccc3)cc(N(c3cccc4c3oc3ccccc34)c3cccc4c3c3ccccc3n4-c3cccc(-c4cccc5ccccc45)c3)c2)cc1 ; c1ccc(-c2ccc(-c3ccccc3)c(N(c3cccc4c3oc3ccccc34)c3cccc4c3c3ccccc3n4-c3cccc(-c4cccc5ccccc45)c3)c2)cc1 ; c1ccc(-c2ccc(N(c3cccc4c3oc3ccccc34)c3cccc4c3c3ccccc3n4-c3cccc(-c4cccc5ccccc45)c3)c(-c3ccccc3)c2)cc1] 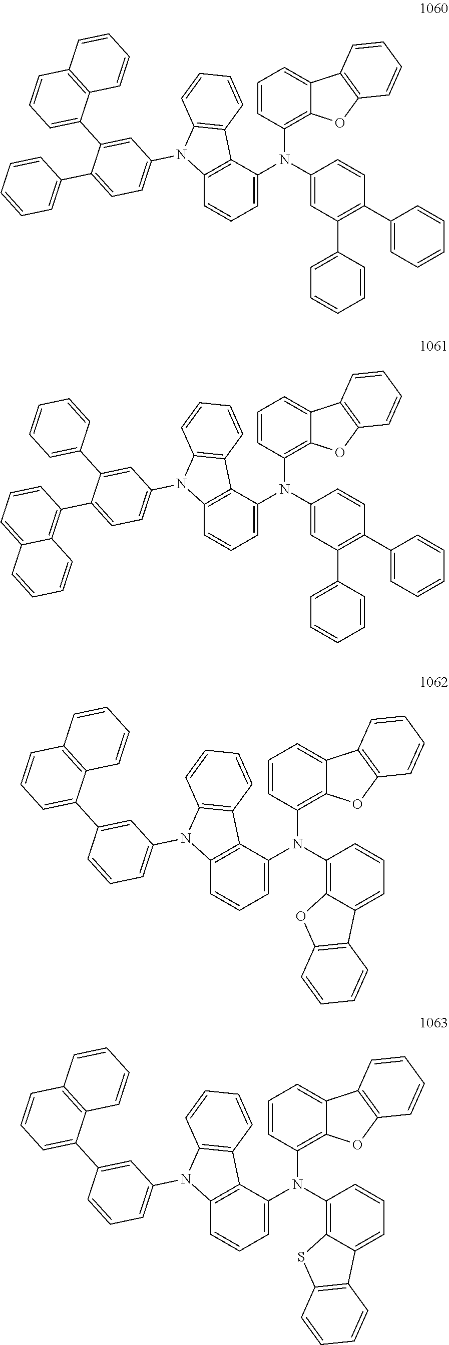 [SMILES: c1ccc(-c2ccc(N(c3cccc4c3oc3ccccc34)c3cccc4c3c3ccccc3n4-c3ccc(-c4ccccc4)c(-c4cccc5ccccc45)c3)cc2-c2ccccc2)cc1 ; c1ccc(-c2ccc(N(c3cccc4c3oc3ccccc34)c3cccc4c3c3ccccc3n4-c3ccc(-c4cccc5ccccc45)c(-c4ccccc4)c3)cc2-c2ccccc2)cc1 ; c1cc(-c2cccc3ccccc23)cc(-n2c3ccccc3c3c(N(c4cccc5c4oc4ccccc45)c4cccc5c4oc4ccccc45)cccc32)c1 ; c1cc(-c2cccc3ccccc23)cc(-n2c3ccccc3c3c(N(c4cccc5c4oc4ccccc45)c4cccc5c4sc4ccccc45)cccc32)c1] 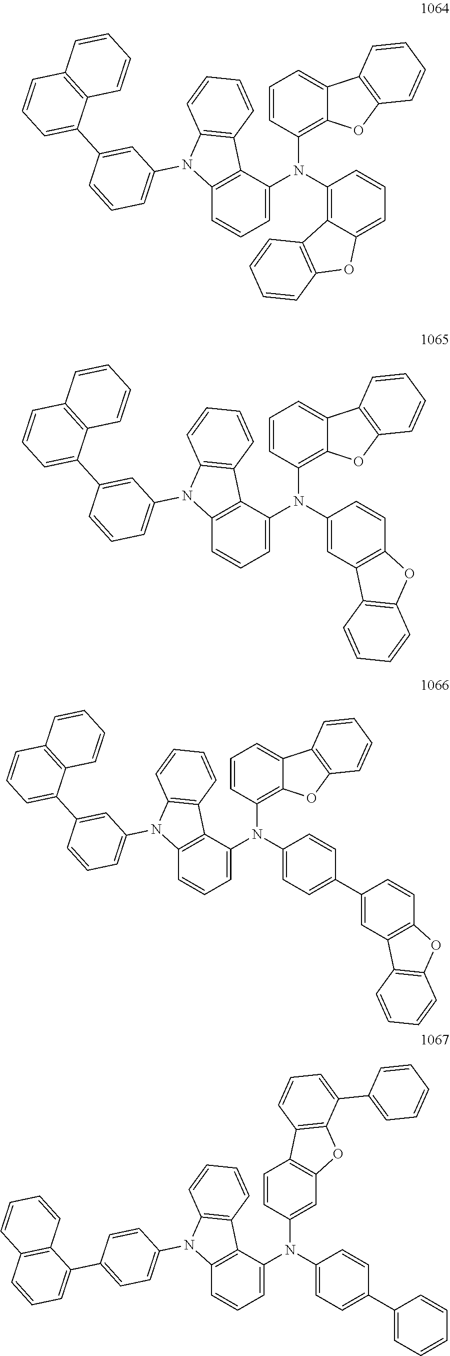 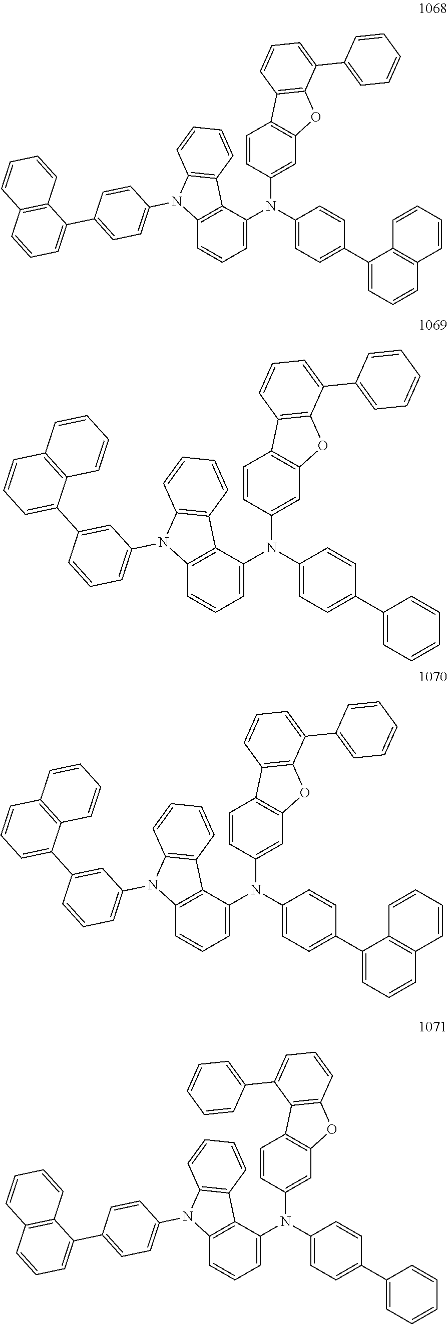 [SMILES: c1ccc(-c2cccc3c2oc2cc(N(c4ccc(-c5cccc6ccccc56)cc4)c4cccc5c4c4ccccc4n5-c4ccc(-c5cccc6ccccc56)cc4)ccc23)cc1 ; c1ccc(-c2ccc(N(c3ccc4c(c3)oc3c(-c5ccccc5)cccc34)c3cccc4c3c3ccccc3n4-c3cccc(-c4cccc5ccccc45)c3)cc2)cc1 ; c1ccc(-c2cccc3c2oc2cc(N(c4ccc(-c5cccc6ccccc56)cc4)c4cccc5c4c4ccccc4n5-c4cccc(-c5cccc6ccccc56)c4)ccc23)cc1 ; c1ccc(-c2ccc(N(c3ccc4c(c3)oc3cccc(-c5ccccc5)c34)c3cccc4c3c3ccccc3n4-c3ccc(-c4cccc5ccccc45)cc3)cc2)cc1] 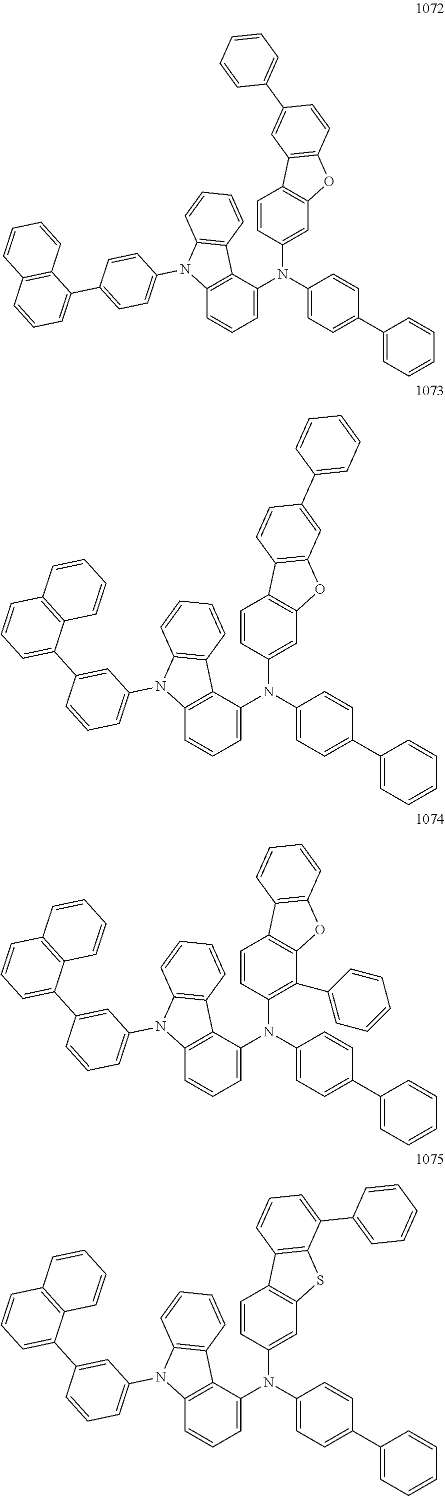 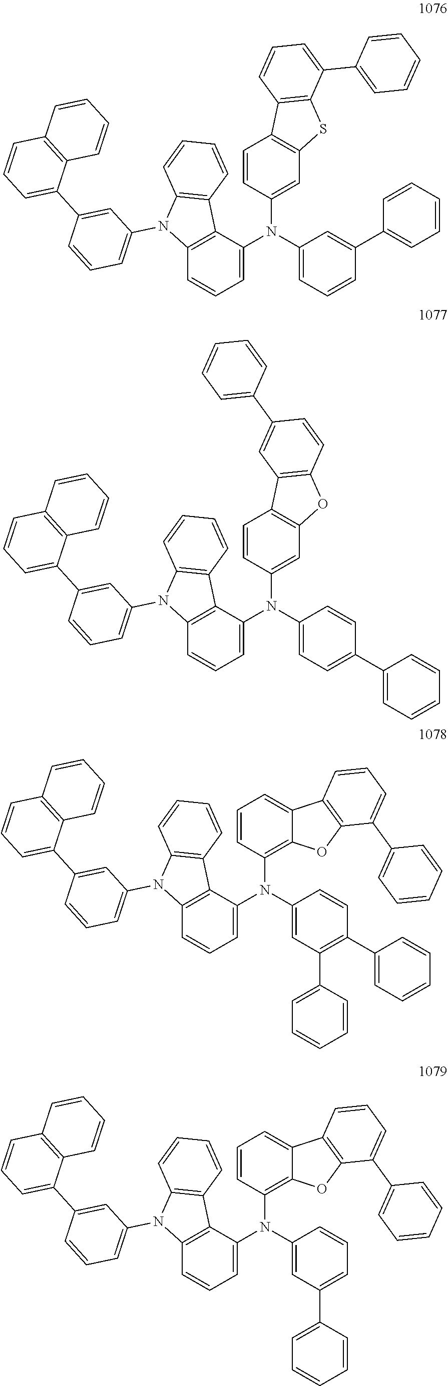 [SMILES: c1ccc(-c2cccc(N(c3ccc4c(c3)sc3c(-c5ccccc5)cccc34)c3cccc4c3c3ccccc3n4-c3cccc(-c4cccc5ccccc45)c3)c2)cc1 ; c1ccc(-c2ccc(N(c3ccc4c(c3)oc3ccc(-c5ccccc5)cc34)c3cccc4c3c3ccccc3n4-c3cccc(-c4cccc5ccccc45)c3)cc2)cc1 ; c1ccc(-c2ccc(N(c3cccc4c3oc3c(-c5ccccc5)cccc34)c3cccc4c3c3ccccc3n4-c3cccc(-c4cccc5ccccc45)c3)cc2-c2ccccc2)cc1 ; c1ccc(-c2cccc(N(c3cccc4c3oc3c(-c5ccccc5)cccc34)c3cccc4c3c3ccccc3n4-c3cccc(-c4cccc5ccccc45)c3)c2)cc1] 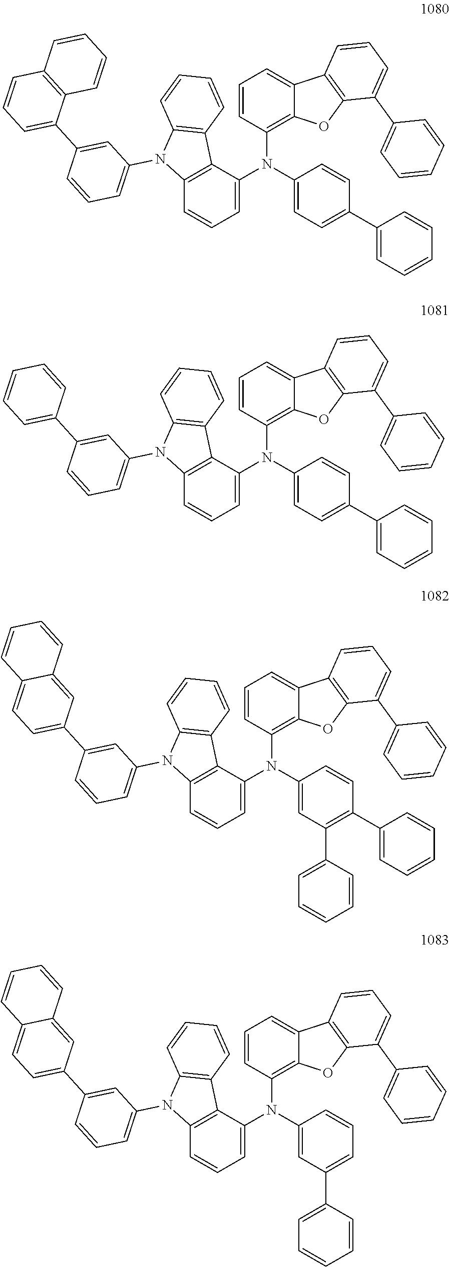 [SMILES: c1ccc(-c2ccc(N(c3cccc4c3oc3c(-c5ccccc5)cccc34)c3cccc4c3c3ccccc3n4-c3cccc(-c4cccc5ccccc45)c3)cc2)cc1 ; c1ccc(-c2ccc(N(c3cccc4c3oc3c(-c5ccccc5)cccc34)c3cccc4c3c3ccccc3n4-c3cccc(-c4ccccc4)c3)cc2)cc1 ; c1ccc(-c2ccc(N(c3cccc4c3oc3c(-c5ccccc5)cccc34)c3cccc4c3c3ccccc3n4-c3cccc(-c4ccc5ccccc5c4)c3)cc2-c2ccccc2)cc1 ; c1ccc(-c2cccc(N(c3cccc4c3oc3c(-c5ccccc5)cccc34)c3cccc4c3c3ccccc3n4-c3cccc(-c4ccc5ccccc5c4)c3)c2)cc1] 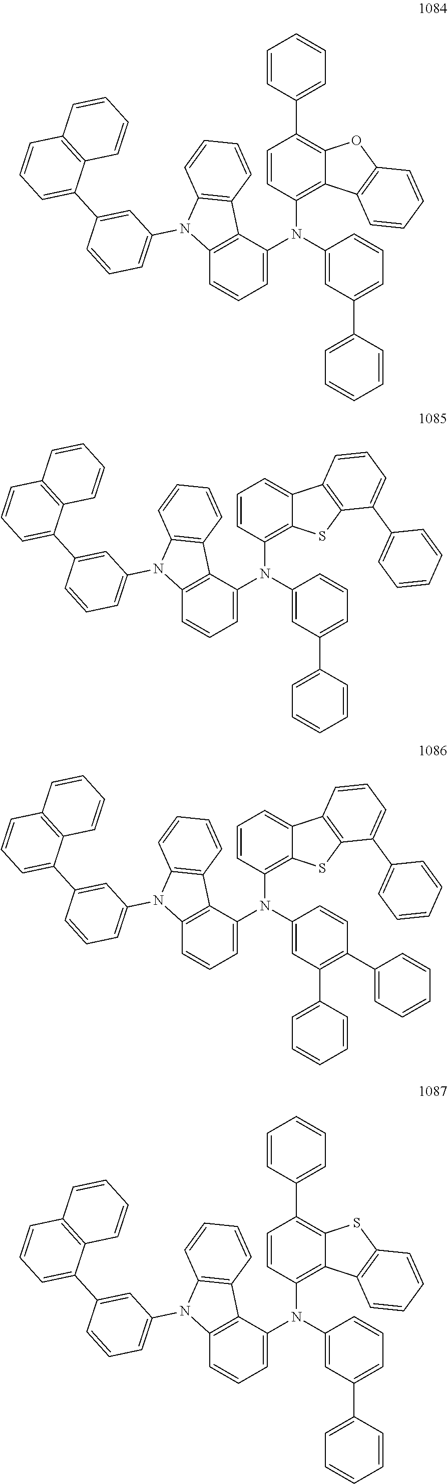 [SMILES: c1ccc(-c2cccc(N(c3ccc(-c4ccccc4)c4oc5ccccc5c34)c3cccc4c3c3ccccc3n4-c3cccc(-c4cccc5ccccc45)c3)c2)cc1 ; c1ccc(-c2cccc(N(c3cccc4c3sc3c(-c5ccccc5)cccc34)c3cccc4c3c3ccccc3n4-c3cccc(-c4cccc5ccccc45)c3)c2)cc1 ; c1ccc(-c2ccc(N(c3cccc4c3sc3c(-c5ccccc5)cccc34)c3cccc4c3c3ccccc3n4-c3cccc(-c4cccc5ccccc45)c3)cc2-c2ccccc2)cc1 ; c1ccc(-c2cccc(N(c3ccc(-c4ccccc4)c4sc5ccccc5c34)c3cccc4c3c3ccccc3n4-c3cccc(-c4cccc5ccccc45)c3)c2)cc1] 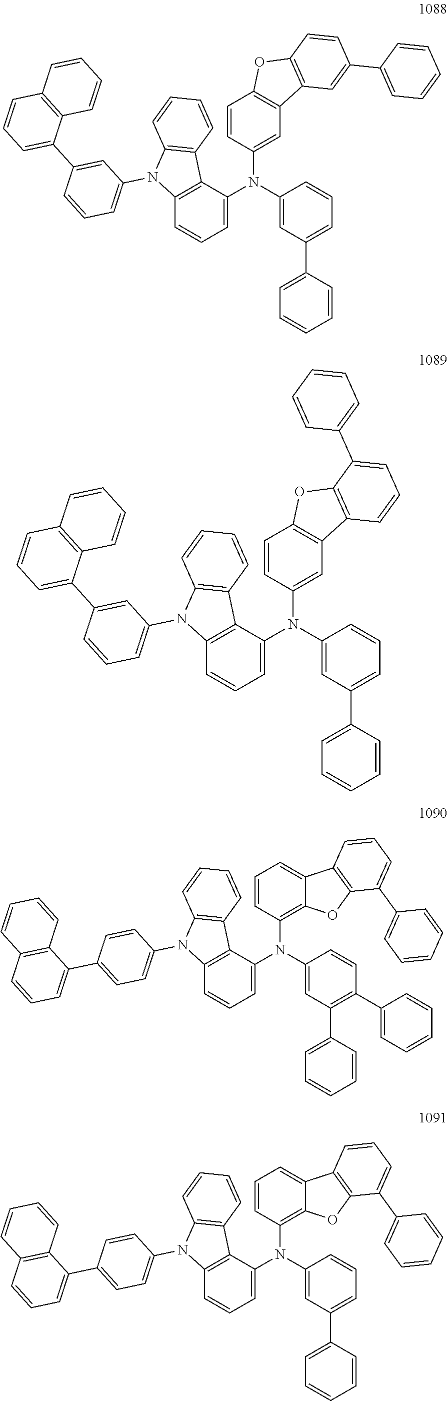 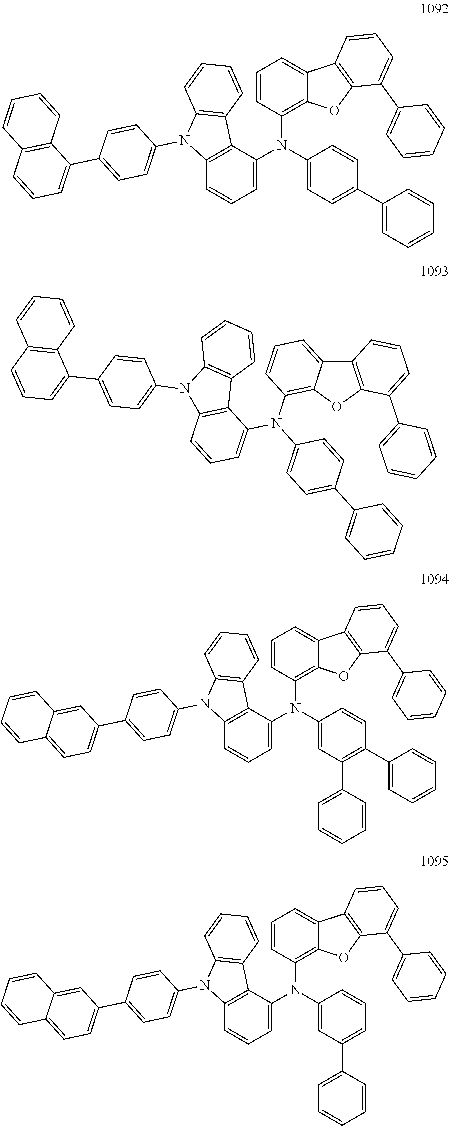 [SMILES: c1ccc(-c2ccc(N(c3cccc4c3oc3c(-c5ccccc5)cccc34)c3cccc4c3c3ccccc3n4-c3ccc(-c4cccc5ccccc45)cc3)cc2)cc1 ; c1ccc(-c2ccc(N(c3cccc4c3oc3c(-c5ccccc5)cccc34)c3cccc4c3c3ccccc3n4-c3ccc(-c4cccc5ccccc45)cc3)cc2)cc1 ; c1ccc(-c2ccc(N(c3cccc4c3oc3c(-c5ccccc5)cccc34)c3cccc4c3c3ccccc3n4-c3ccc(-c4ccc5ccccc5c4)cc3)cc2-c2ccccc2)cc1 ; c1ccc(-c2cccc(N(c3cccc4c3oc3c(-c5ccccc5)cccc34)c3cccc4c3c3ccccc3n4-c3ccc(-c4ccc5ccccc5c4)cc3)c2)cc1] 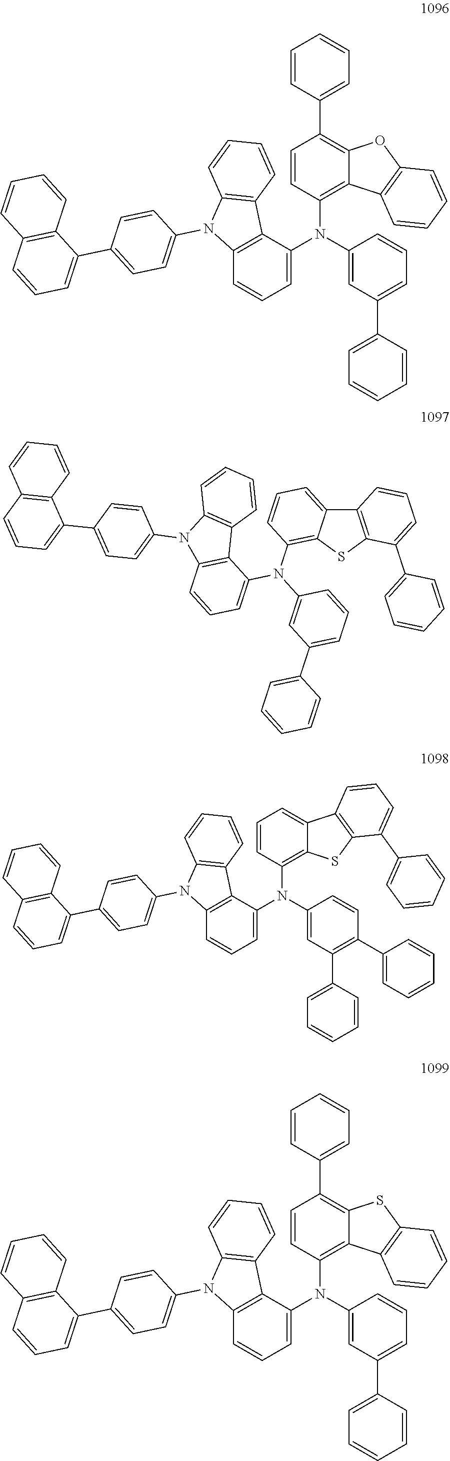 [SMILES: c1ccc(-c2cccc(N(c3ccc(-c4ccccc4)c4oc5ccccc5c34)c3cccc4c3c3ccccc3n4-c3ccc(-c4cccc5ccccc45)cc3)c2)cc1 ; c1ccc(-c2cccc(N(c3cccc4c3sc3c(-c5ccccc5)cccc34)c3cccc4c3c3ccccc3n4-c3ccc(-c4cccc5ccccc45)cc3)c2)cc1 ; c1ccc(-c2ccc(N(c3cccc4c3sc3c(-c5ccccc5)cccc34)c3cccc4c3c3ccccc3n4-c3ccc(-c4cccc5ccccc45)cc3)cc2-c2ccccc2)cc1 ; c1ccc(-c2cccc(N(c3ccc(-c4ccccc4)c4sc5ccccc5c34)c3cccc4c3c3ccccc3n4-c3ccc(-c4cccc5ccccc45)cc3)c2)cc1] 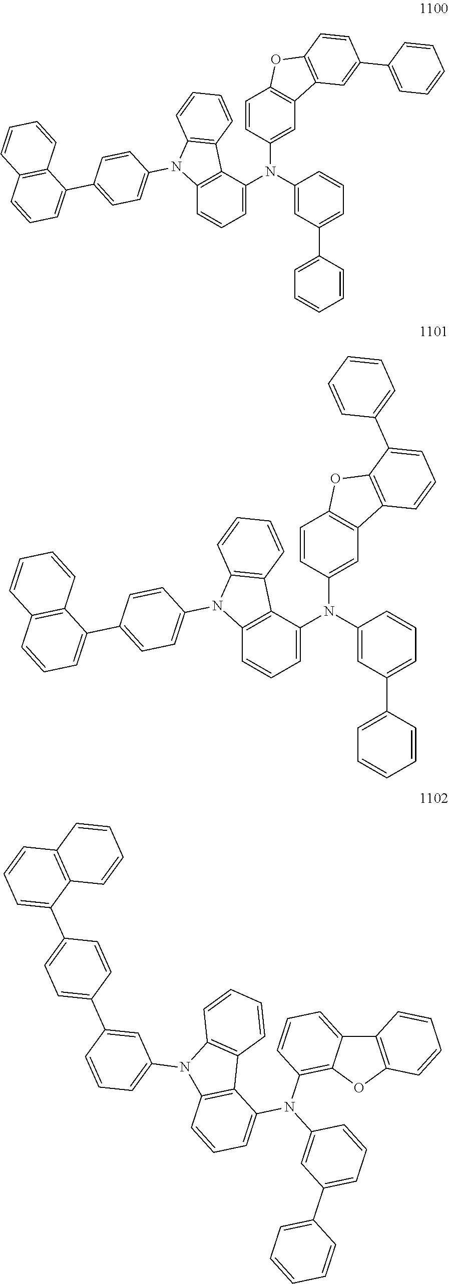 [SMILES: c1ccc(-c2cccc(N(c3ccc4oc5ccc(-c6ccccc6)cc5c4c3)c3cccc4c3c3ccccc3n4-c3ccc(-c4cccc5ccccc45)cc3)c2)cc1 ; c1ccc(-c2cccc(N(c3ccc4oc5c(-c6ccccc6)cccc5c4c3)c3cccc4c3c3ccccc3n4-c3ccc(-c4cccc5ccccc45)cc3)c2)cc1 ; c1ccc(-c2cccc(N(c3cccc4c3oc3ccccc34)c3cccc4c3c3ccccc3n4-c3cccc(-c4ccc(-c5cccc6ccccc56)cc4)c3)c2)cc1] 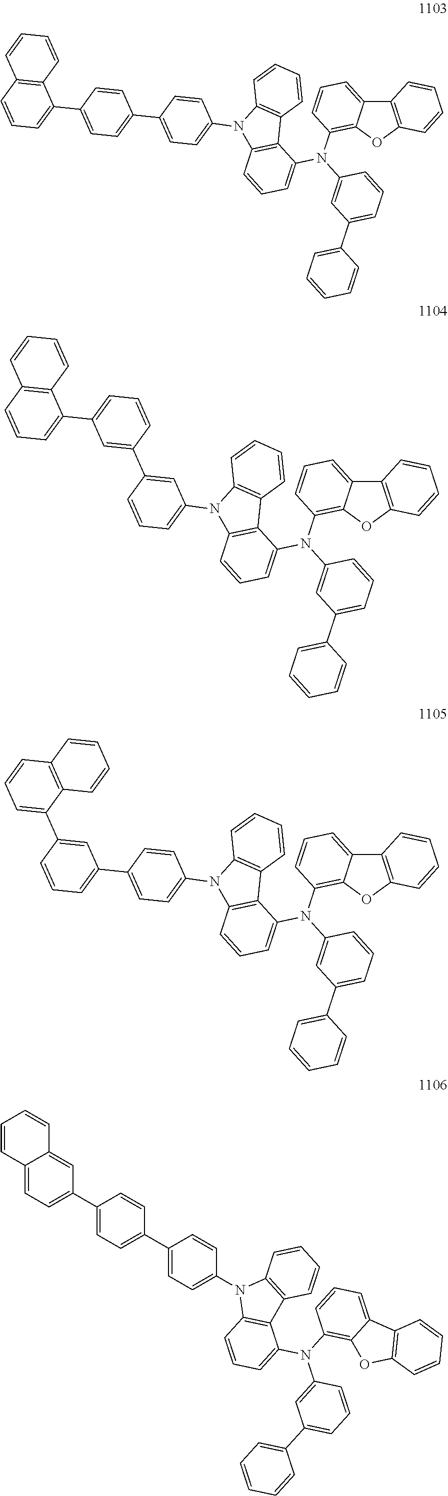 [SMILES: c1ccc(-c2cccc(N(c3cccc4c3oc3ccccc34)c3cccc4c3c3ccccc3n4-c3ccc(-c4ccc(-c5cccc6ccccc56)cc4)cc3)c2)cc1 ; c1ccc(-c2cccc(N(c3cccc4c3oc3ccccc34)c3cccc4c3c3ccccc3n4-c3cccc(-c4cccc(-c5cccc6ccccc56)c4)c3)c2)cc1 ; c1ccc(-c2cccc(N(c3cccc4c3oc3ccccc34)c3cccc4c3c3ccccc3n4-c3ccc(-c4cccc(-c5cccc6ccccc56)c4)cc3)c2)cc1 ; c1ccc(-c2cccc(N(c3cccc4c3oc3ccccc34)c3cccc4c3c3ccccc3n4-c3ccc(-c4ccc(-c5ccc6ccccc6c5)cc4)cc3)c2)cc1] 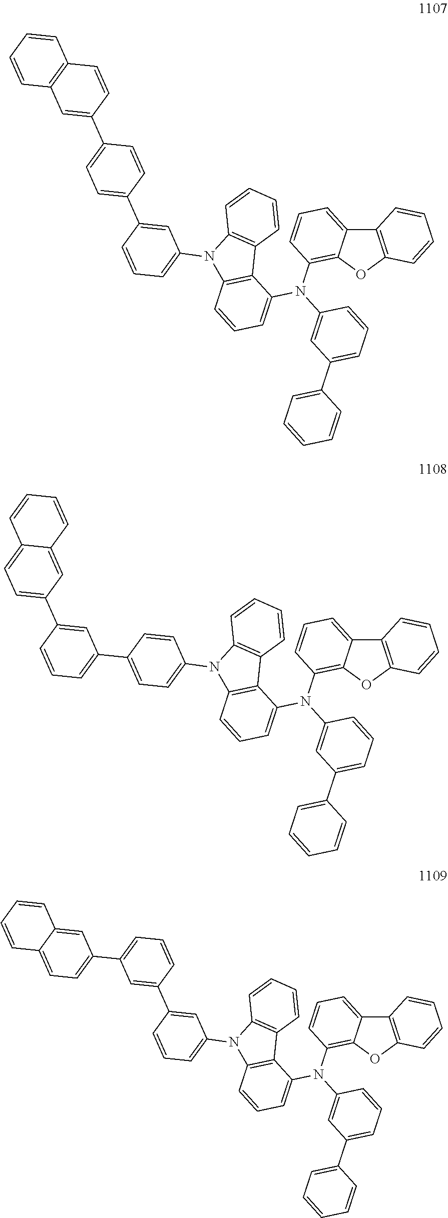 [SMILES: c1ccc(-c2cccc(N(c3cccc4c3oc3ccccc34)c3cccc4c3c3ccccc3n4-c3cccc(-c4ccc(-c5ccc6ccccc6c5)cc4)c3)c2)cc1 ; c1ccc(-c2cccc(N(c3cccc4c3oc3ccccc34)c3cccc4c3c3ccccc3n4-c3ccc(-c4cccc(-c5ccc6ccccc6c5)c4)cc3)c2)cc1 ; c1ccc(-c2cccc(N(c3cccc4c3oc3ccccc34)c3cccc4c3c3ccccc3n4-c3cccc(-c4cccc(-c5ccc6ccccc6c5)c4)c3)c2)cc1] 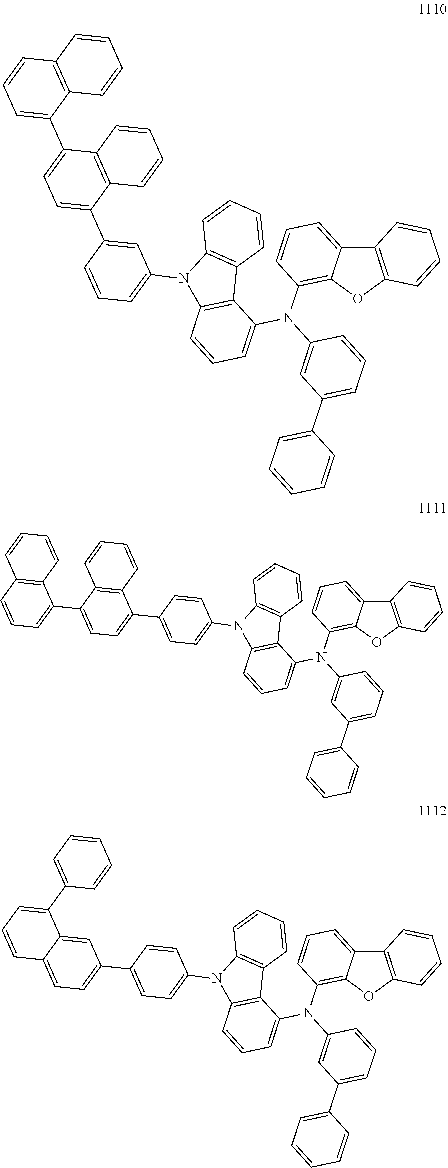 [SMILES: c1ccc(-c2cccc(N(c3cccc4c3oc3ccccc34)c3cccc4c3c3ccccc3n4-c3cccc(-c4ccc(-c5cccc6ccccc56)c5ccccc45)c3)c2)cc1 ; c1ccc(-c2cccc(N(c3cccc4c3oc3ccccc34)c3cccc4c3c3ccccc3n4-c3ccc(-c4ccc(-c5cccc6ccccc56)c5ccccc45)cc3)c2)cc1 ; c1ccc(-c2cccc(N(c3cccc4c3oc3ccccc34)c3cccc4c3c3ccccc3n4-c3ccc(-c4ccc5cccc(-c6ccccc6)c5c4)cc3)c2)cc1] 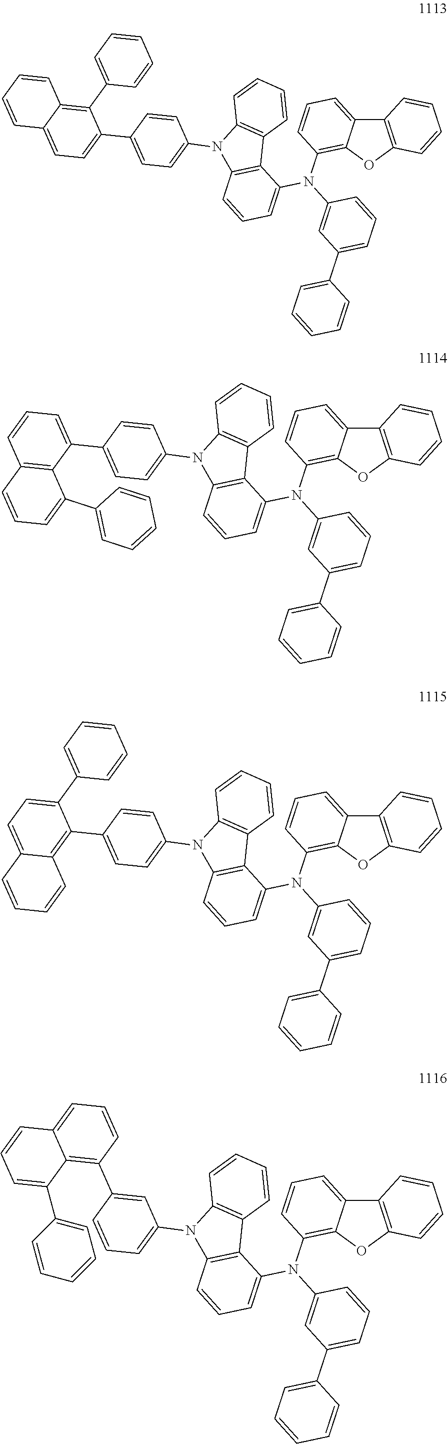 [SMILES: c1ccc(-c2cccc(N(c3cccc4c3oc3ccccc34)c3cccc4c3c3ccccc3n4-c3ccc(-c4ccc5ccccc5c4-c4ccccc4)cc3)c2)cc1 ; c1ccc(-c2cccc(N(c3cccc4c3oc3ccccc34)c3cccc4c3c3ccccc3n4-c3ccc(-c4cccc5cccc(-c6ccccc6)c45)cc3)c2)cc1 ; c1ccc(-c2cccc(N(c3cccc4c3oc3ccccc34)c3cccc4c3c3ccccc3n4-c3ccc(-c4c(-c5ccccc5)ccc5ccccc45)cc3)c2)cc1 ; c1ccc(-c2cccc(N(c3cccc4c3oc3ccccc34)c3cccc4c3c3ccccc3n4-c3cccc(-c4cccc5cccc(-c6ccccc6)c45)c3)c2)cc1] 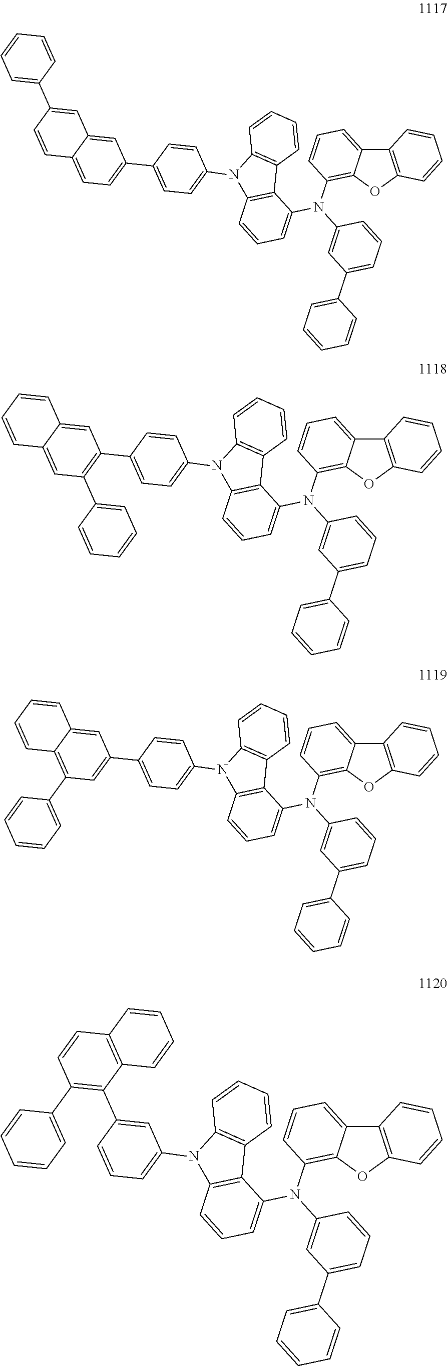 [SMILES: c1ccc(-c2cccc(N(c3cccc4c3oc3ccccc34)c3cccc4c3c3ccccc3n4-c3ccc(-c4ccc5ccc(-c6ccccc6)cc5c4)cc3)c2)cc1 ; c1ccc(-c2cccc(N(c3cccc4c3oc3ccccc34)c3cccc4c3c3ccccc3n4-c3ccc(-c4cc5ccccc5cc4-c4ccccc4)cc3)c2)cc1 ; c1ccc(-c2cccc(N(c3cccc4c3oc3ccccc34)c3cccc4c3c3ccccc3n4-c3ccc(-c4cc(-c5ccccc5)c5ccccc5c4)cc3)c2)cc1 ; c1ccc(-c2cccc(N(c3cccc4c3oc3ccccc34)c3cccc4c3c3ccccc3n4-c3cccc(-c4c(-c5ccccc5)ccc5ccccc45)c3)c2)cc1] 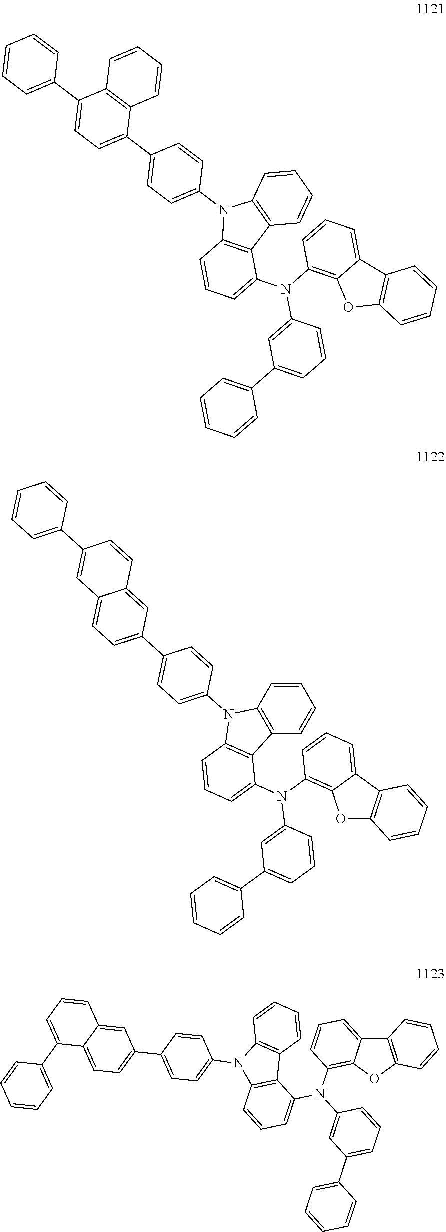 [SMILES: c1ccc(-c2cccc(N(c3cccc4c3oc3ccccc34)c3cccc4c3c3ccccc3n4-c3ccc(-c4ccc(-c5ccccc5)c5ccccc45)cc3)c2)cc1 ; c1ccc(-c2cccc(N(c3cccc4c3oc3ccccc34)c3cccc4c3c3ccccc3n4-c3ccc(-c4ccc5cc(-c6ccccc6)ccc5c4)cc3)c2)cc1 ; c1ccc(-c2cccc(N(c3cccc4c3oc3ccccc34)c3cccc4c3c3ccccc3n4-c3ccc(-c4ccc5c(-c6ccccc6)cccc5c4)cc3)c2)cc1] 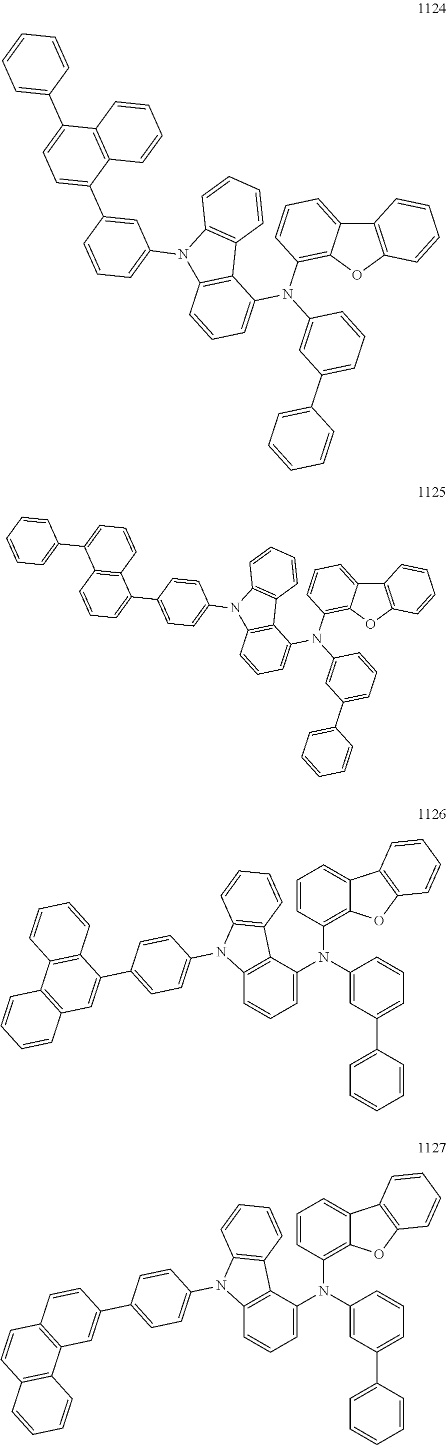 [SMILES: c1ccc(-c2cccc(N(c3cccc4c3oc3ccccc34)c3cccc4c3c3ccccc3n4-c3cccc(-c4ccc(-c5ccccc5)c5ccccc45)c3)c2)cc1 ; c1ccc(-c2cccc(N(c3cccc4c3oc3ccccc34)c3cccc4c3c3ccccc3n4-c3ccc(-c4cccc5c(-c6ccccc6)cccc45)cc3)c2)cc1 ; c1ccc(-c2cccc(N(c3cccc4c3oc3ccccc34)c3cccc4c3c3ccccc3n4-c3ccc(-c4cc5ccccc5c5ccccc45)cc3)c2)cc1 ; c1ccc(-c2cccc(N(c3cccc4c3oc3ccccc34)c3cccc4c3c3ccccc3n4-c3ccc(-c4ccc5ccc6ccccc6c5c4)cc3)c2)cc1] 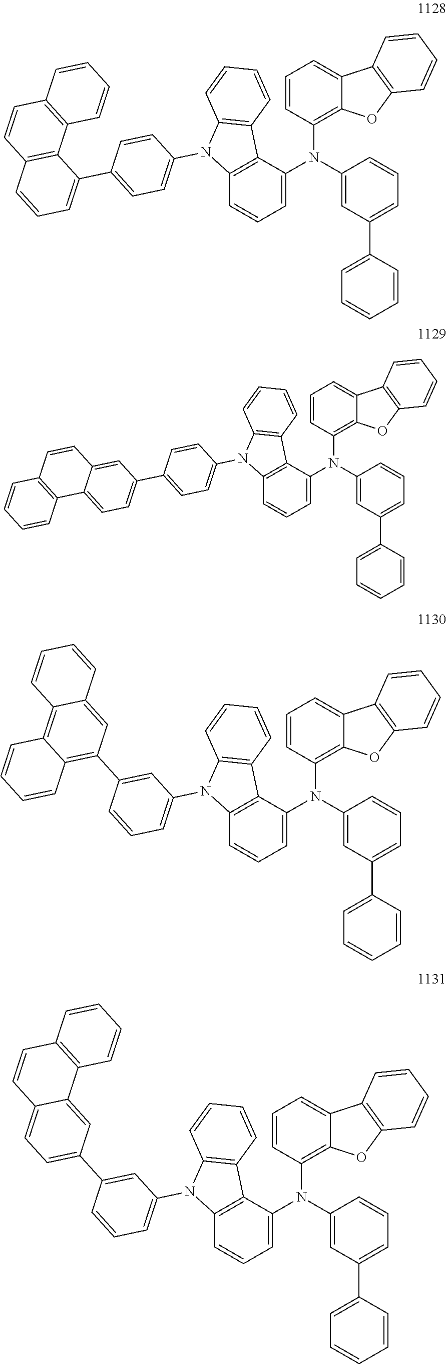 [SMILES: c1ccc(-c2cccc(N(c3cccc4c3oc3ccccc34)c3cccc4c3c3ccccc3n4-c3ccc(-c4cccc5ccc6ccccc6c45)cc3)c2)cc1 ; c1ccc(-c2cccc(N(c3cccc4c3oc3ccccc34)c3cccc4c3c3ccccc3n4-c3ccc(-c4ccc5c(ccc6ccccc65)c4)cc3)c2)cc1 ; c1ccc(-c2cccc(N(c3cccc4c3oc3ccccc34)c3cccc4c3c3ccccc3n4-c3cccc(-c4cc5ccccc5c5ccccc45)c3)c2)cc1 ; c1ccc(-c2cccc(N(c3cccc4c3oc3ccccc34)c3cccc4c3c3ccccc3n4-c3cccc(-c4ccc5ccc6ccccc6c5c4)c3)c2)cc1] 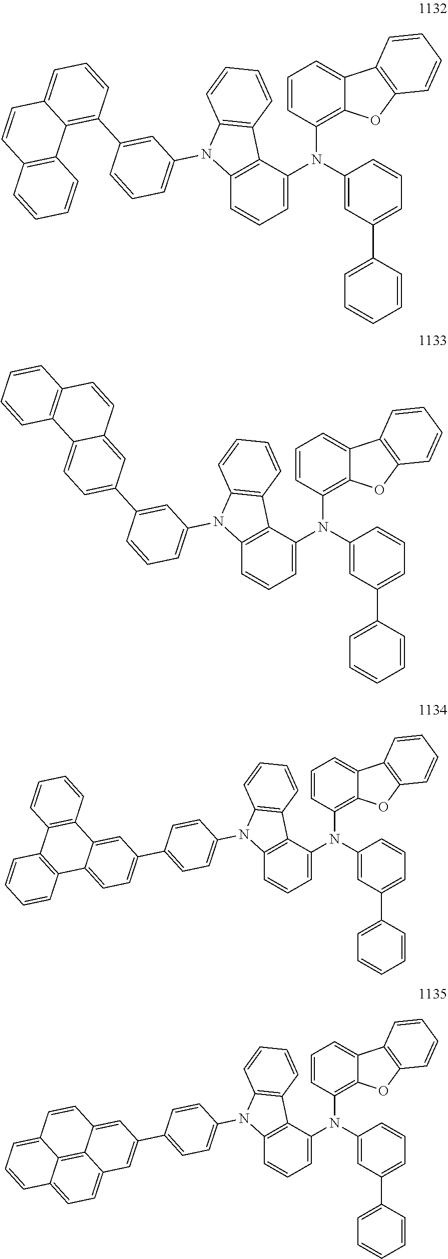 [SMILES: c1ccc(-c2cccc(N(c3cccc4c3oc3ccccc34)c3cccc4c3c3ccccc3n4-c3cccc(-c4cccc5ccc6ccccc6c45)c3)c2)cc1 ; c1ccc(-c2cccc(N(c3cccc4c3oc3ccccc34)c3cccc4c3c3ccccc3n4-c3cccc(-c4ccc5c(ccc6ccccc65)c4)c3)c2)cc1 ; c1ccc(-c2cccc(N(c3cccc4c3oc3ccccc34)c3cccc4c3c3ccccc3n4-c3ccc(-c4ccc5c6ccccc6c6ccccc6c5c4)cc3)c2)cc1 ; c1ccc(-c2cccc(N(c3cccc4c3oc3ccccc34)c3cccc4c3c3ccccc3n4-c3ccc(-c4cc5ccc6cccc7ccc(c4)c5c67)cc3)c2)cc1] 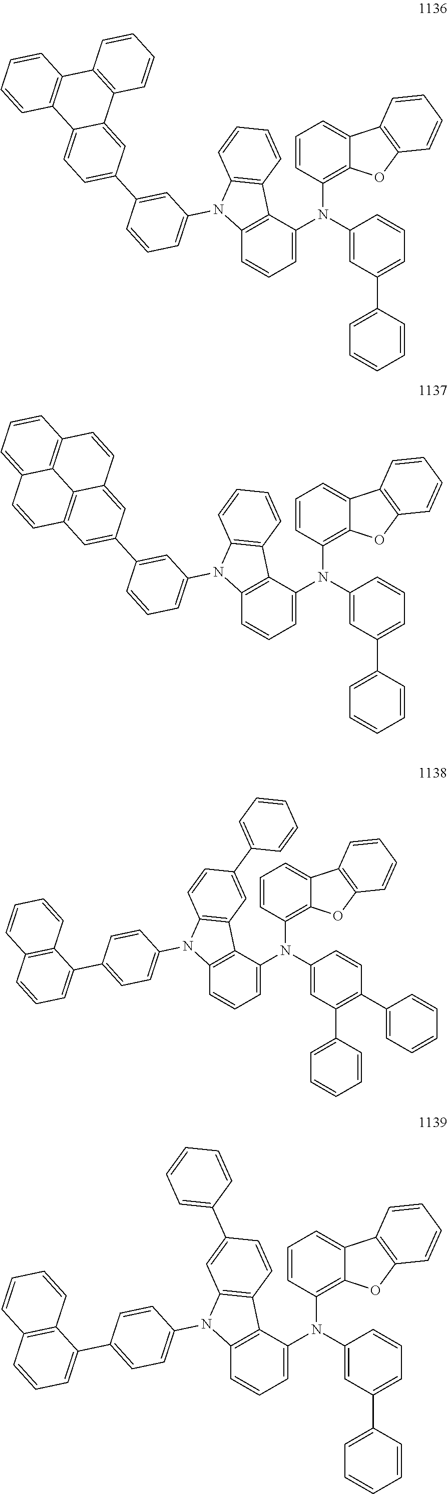 [SMILES: c1ccc(-c2cccc(N(c3cccc4c3oc3ccccc34)c3cccc4c3c3ccccc3n4-c3cccc(-c4ccc5c6ccccc6c6ccccc6c5c4)c3)c2)cc1 ; c1ccc(-c2cccc(N(c3cccc4c3oc3ccccc34)c3cccc4c3c3ccccc3n4-c3cccc(-c4cc5ccc6cccc7ccc(c4)c5c67)c3)c2)cc1 ; c1ccc(-c2ccc3c(c2)c2c(N(c4ccc(-c5ccccc5)c(-c5ccccc5)c4)c4cccc5c4oc4ccccc45)cccc2n3-c2ccc(-c3cccc4ccccc34)cc2)cc1 ; c1ccc(-c2cccc(N(c3cccc4c3oc3ccccc34)c3cccc4c3c3ccc(-c5ccccc5)cc3n4-c3ccc(-c4cccc5ccccc45)cc3)c2)cc1] 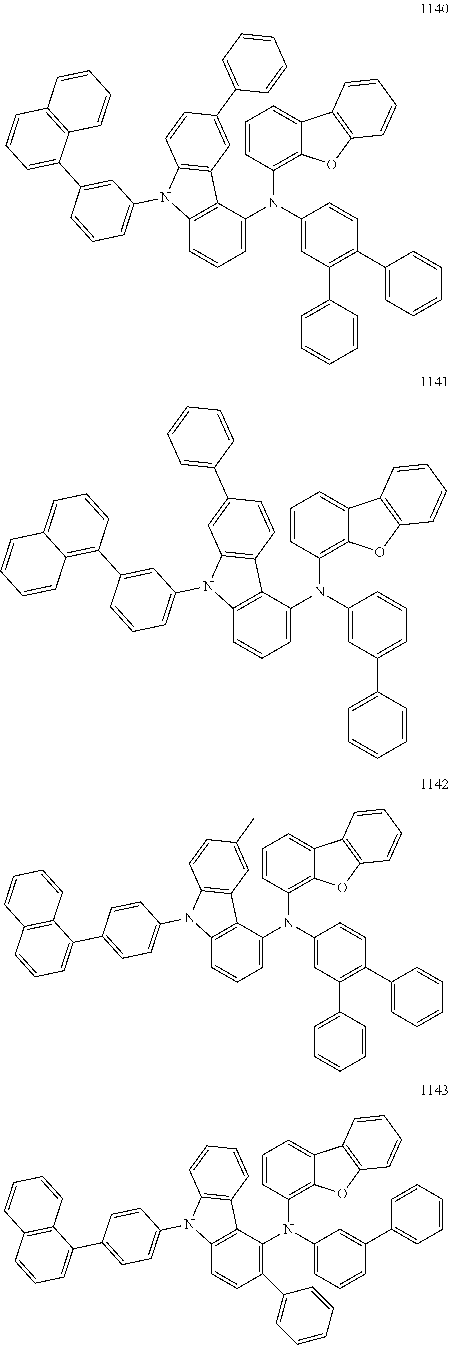 [SMILES: c1ccc(-c2ccc3c(c2)c2c(N(c4ccc(-c5ccccc5)c(-c5ccccc5)c4)c4cccc5c4oc4ccccc45)cccc2n3-c2cccc(-c3cccc4ccccc34)c2)cc1 ; c1ccc(-c2cccc(N(c3cccc4c3oc3ccccc34)c3cccc4c3c3ccc(-c5ccccc5)cc3n4-c3cccc(-c4cccc5ccccc45)c3)c2)cc1 ; Cc1ccc2c(c1)c1c(N(c3ccc(-c4ccccc4)c(-c4ccccc4)c3)c3cccc4c3oc3ccccc34)cccc1n2-c1ccc(-c2cccc3ccccc23)cc1 ; c1ccc(-c2cccc(N(c3cccc4c3oc3ccccc34)c3c(-c4ccccc4)ccc4c3c3ccccc3n4-c3ccc(-c4cccc5ccccc45)cc3)c2)cc1] 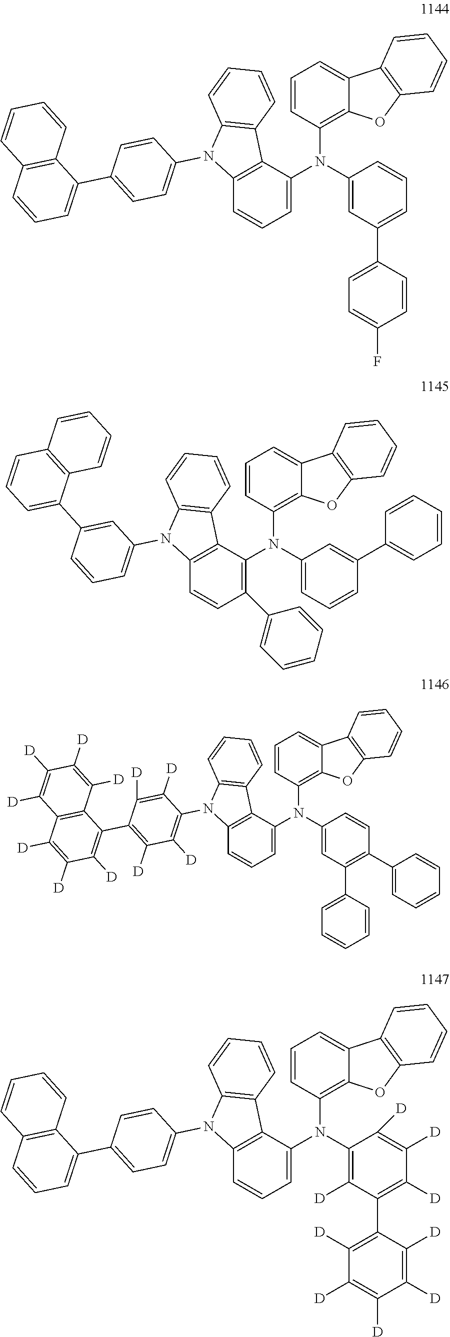 [SMILES: Fc1ccc(-c2cccc(N(c3cccc4c3oc3ccccc34)c3cccc4c3c3ccccc3n4-c3ccc(-c4cccc5ccccc45)cc3)c2)cc1 ; c1ccc(-c2cccc(N(c3cccc4c3oc3ccccc34)c3c(-c4ccccc4)ccc4c3c3ccccc3n4-c3cccc(-c4cccc5ccccc45)c3)c2)cc1 ; [2H]c1c([2H])c(-n2c3ccccc3c3c(N(c4ccc(-c5ccccc5)c(-c5ccccc5)c4)c4cccc5c4oc4ccccc45)cccc32)c([2H])c([2H])c1-c1c([2H])c([2H])c([2H])c2c([2H])c([2H])c([2H])c([2H])c12 ; [2H]c1c([2H])c([2H])c(-c2c([2H])c([2H])c([2H])c(N(c3cccc4c3oc3ccccc34)c3cccc4c3c3ccccc3n4-c3ccc(-c4cccc5ccccc45)cc3)c2[2H])c([2H])c1[2H]] 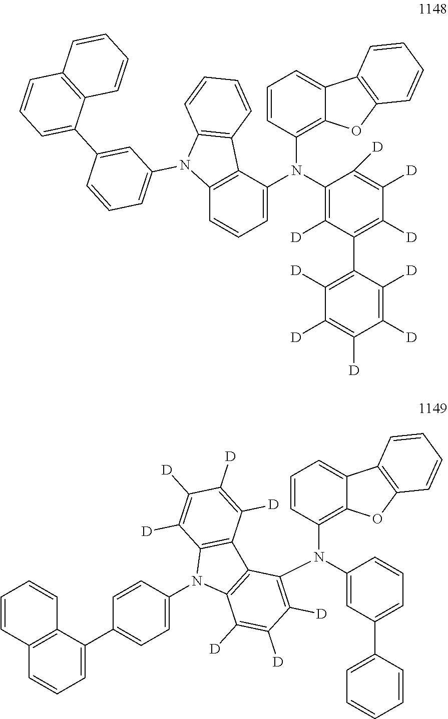 [SMILES: [2H]c1c([2H])c([2H])c(-c2c([2H])c([2H])c([2H])c(N(c3cccc4c3oc3ccccc34)c3cccc4c3c3ccccc3n4-c3cccc(-c4cccc5ccccc45)c3)c2[2H])c([2H])c1[2H] ; [2H]c1c([2H])c([2H])c2c(c1[2H])c1c(N(c3cccc(-c4ccccc4)c3)c3cccc4c3oc3ccccc34)c([2H])c([2H])c([2H])c1n2-c1ccc(-c2cccc3ccccc23)cc1]
in Compound Group 1, D is a deuterium atom.

14. An amine compound, the amine compound represented by Formula 1: 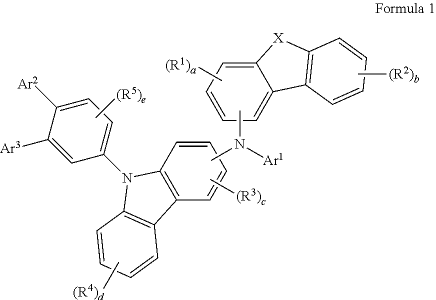
in Formula 1,
X is O or S,
Ar¹ is a substituted or unsubstituted aryl group of 6 to 30 ring-forming carbon atoms, or a substituted or unsubstituted heteroaryl group of 2 to 30 ring-forming carbon atoms,
Ar² and Ar³ are each independently a hydrogen atom, a deuterium atom, or a substituted or unsubstituted aryl group of 6 to 30 ring-forming carbon atoms, where at least one selected from among Ar² and Ar³ is a substituted or unsubstituted aryl group of 10 to 30 ring-forming carbon atoms,
R¹ to R⁵ are each independently a hydrogen atom, a deuterium atom, a halogen atom, a substituted or unsubstituted alkyl group of 1 to 15 carbon atoms, a substituted or unsubstituted aryl group of 6 to 30 ring-forming carbon atoms, or a substituted or unsubstituted heteroaryl group of 2 to 30 ring-forming carbon atoms,
“a”, “c” and “e” are each independently an integer of 0 to 3,
“b” and “d” are each independently an integer of 0 to 4,
when
a nitrogen atom of the amine compound is connected with position 2 or position 6 of a carbazole moiety, and is connected with position 3 or position 6 of a dibenzothiophene moiety or a dibenzofuran moiety, or
the nitrogen atom of the amine compound is connected with position 4 or position 5 of the carbazole moiety, and is connected with position 3 or position 6 of the dibenzothiophene moiety or the dibenzofuran moiety, then
a+b≥1, and at least one selected from among R¹ and R² is not a hydrogen atom, and
when the nitrogen atom of the amine compound is connected with position 3 of the carbazole moiety, then R³ is a hydrogen atom or a deuterium atom, and
Ar¹ comprises a moiety of 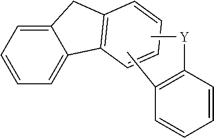
and Y is O or S, is excluded.

15. The amine compound of claim 14, wherein the amine compound is represented by Formula 2-1 or Formula 2-2: 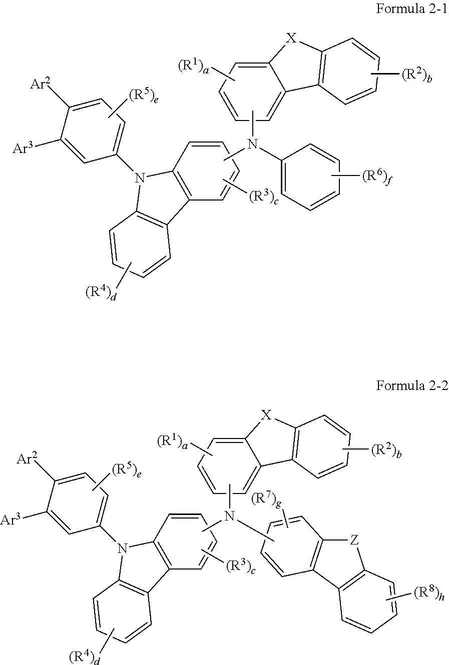
in Formula 2-1,
R⁶ is a hydrogen atom, a deuterium atom, a substituted or unsubstituted aryl group of 6 to 30 ring-forming carbon atoms, or a substituted or unsubstituted heteroaryl group of 2 to 30 ring-forming carbon atoms, and
“f” is an integer of 0 to 5,
in Formula 2-2,
Z is O or S,
R⁷ and R⁸ are each independently a hydrogen atom or a deuterium atom,
“g” is an integer of 0 to 3, and “h” is an integer of 0 to 4, and
in Formula 2-1 and Formula 2-2,
X, Ar², Ar³, R¹ to R⁵, and “a” to “e” are as defined in Formula 1.

16. The amine compound of claim 14, wherein the amine compound is represented by Formula 3-1 or Formula 3-2: 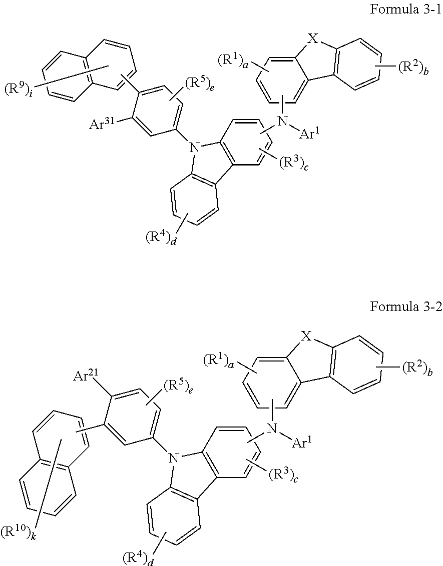
in Formula 3-1 and Formula 3-2,
R⁹, R¹⁰, Ar²¹ and Ar³¹ are each independently a hydrogen atom, a deuterium atom, or a substituted or unsubstituted aryl group of 6 to 30 ring-forming carbon atoms,
“i” and “k” are each independently an integer of 0 to 7, and
X, Ar¹, R¹ to R⁵, and “a” to “e” are as defined in Formula 1.

17. The amine compound of claim 14, wherein the amine compound is represented by any one selected from among Formula 4-1 to Formula 4-4: 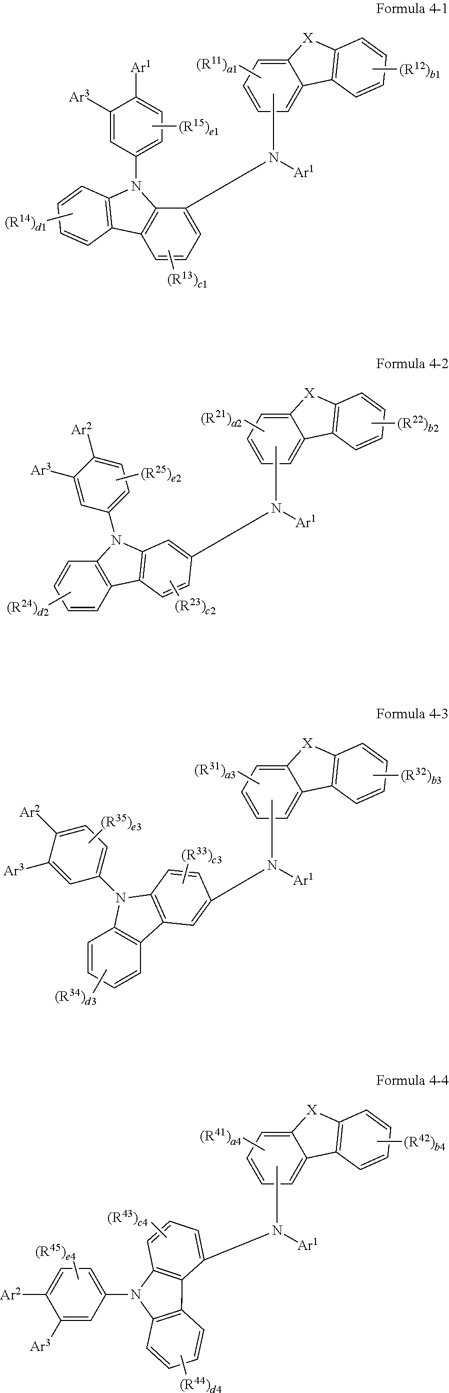
in Formula 4-1 to Formula 4-4,
R¹¹ to R¹³, R²¹ to R²³, R³¹ to R³³, R⁴¹, and R⁴² are each independently a hydrogen atom, a deuterium atom, or a substituted or unsubstituted aryl group of 6 to ring-forming carbon atoms,
R¹⁴, R²⁴, R³⁴, and R⁴⁴ are each independently a hydrogen atom, a deuterium atom, a halogen atom, a substituted or unsubstituted alkyl group of 1 to 15 carbon atoms, or a substituted or unsubstituted aryl group of 6 to 30 ring-forming carbon atoms,
R¹⁵, R²⁵, R³⁵, R⁴³ and R⁴⁵ are each independently a hydrogen atom, or a deuterium atom,
a1 to a4, c1 to c4, and e1 to e4 are each independently an integer of 0 to 3,
b1 to b4, and d1 to d4 are each independently an integer of 0 to 4,
X, and Ar¹ to Ar³ are as defined in Formula 1,
in Formula 4-2 when the nitrogen atom of the amine compound is connected with position 3 or position 6 of a dibenzothiophene moiety or a dibenzofuran moiety, then a2+b2≥1, and at least one selected from among R²¹ and R²² is a substituted or unsubstituted aryl group of 6 to 30 ring-forming carbon atoms, and
in Formula 4-4 when the nitrogen atom of the amine compound is connected with position 3 or position 6 of a dibenzothiophene moiety or a dibenzofuran moiety, then a4+b4≥1, and at least one selected from among R⁴¹ and R⁴² is a substituted or unsubstituted aryl group of 6 to 30 ring-forming carbon atoms.

18. The amine compound of claim 14, wherein at least one selected from among Ar² and Ar³ is represented by any one selected from among substituents in Substituent Group 1: 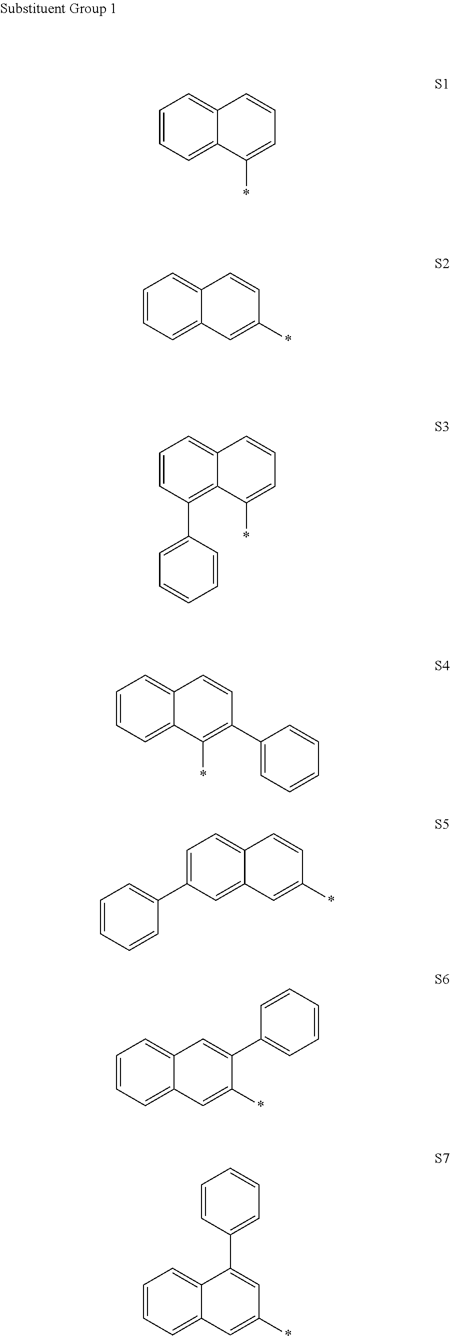 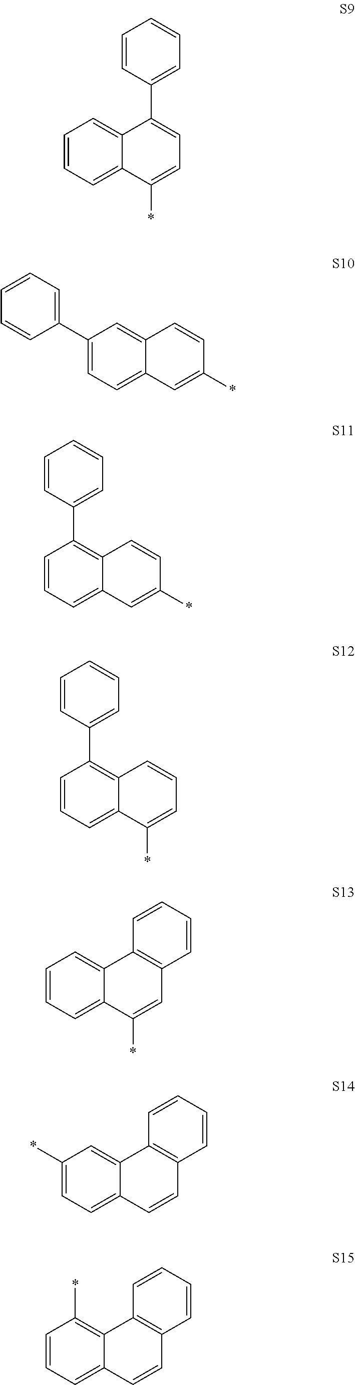 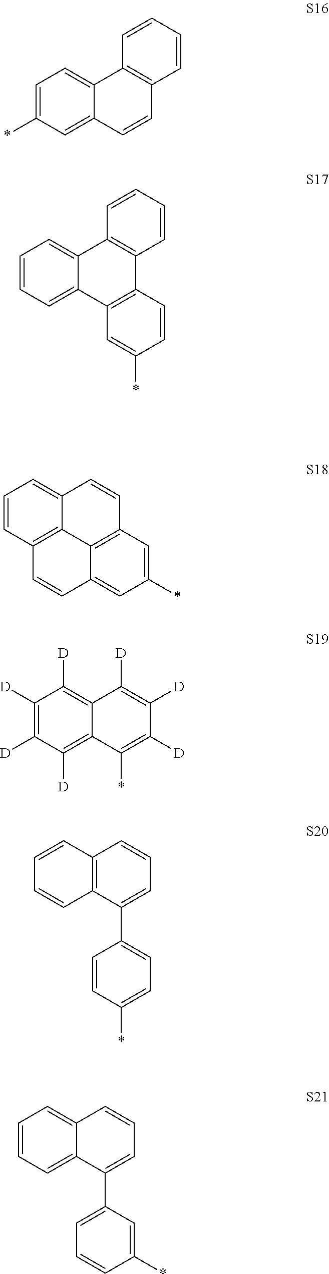 [SMILES: *c1ccc2c(ccc3ccccc32)c1 ; *c1ccc2c3ccccc3c3ccccc3c2c1 ; *c1cc2ccc3cccc4ccc(c1)c2c34 ; *c1c([2H])c([2H])c([2H])c2c([2H])c([2H])c([2H])c([2H])c12 ; *c1ccc(-c2cccc3ccccc23)cc1 ; *c1cccc(-c2cccc3ccccc23)c1] 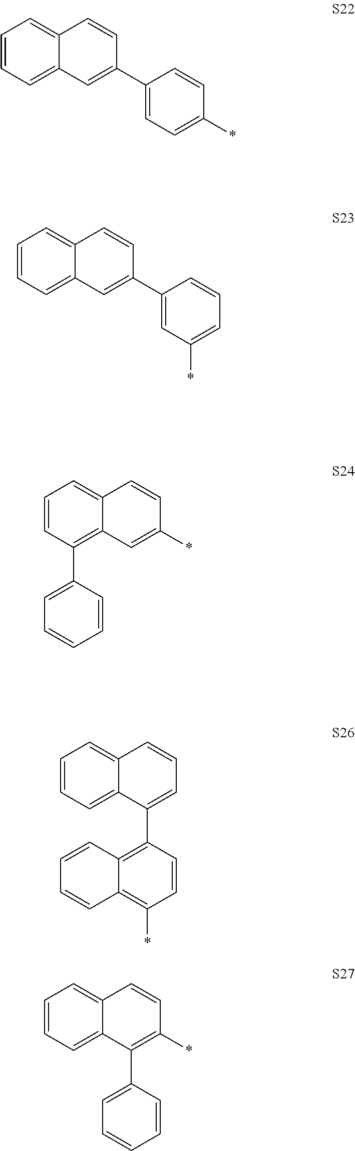
in Substituent Group 1, D is a deuterium atom.

19. The amine compound of claim 14, wherein the amine compound is represented by any one selected from among compounds in Compound Group 1: 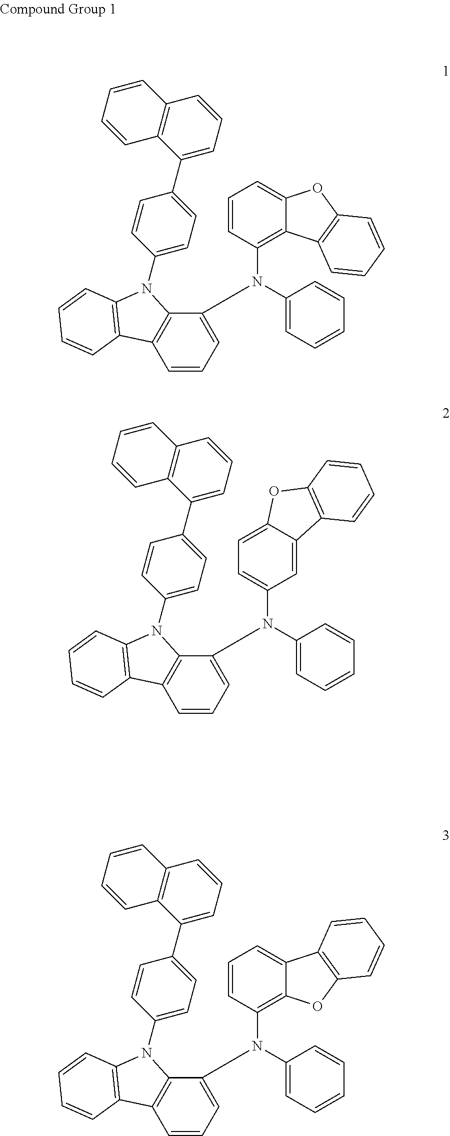 [SMILES: c1ccc(N(c2cccc3oc4ccccc4c23)c2cccc3c4ccccc4n(-c4ccc(-c5cccc6ccccc56)cc4)c23)cc1 ; c1ccc(N(c2ccc3oc4ccccc4c3c2)c2cccc3c4ccccc4n(-c4ccc(-c5cccc6ccccc56)cc4)c23)cc1 ; c1ccc(N(c2cccc3c2oc2ccccc23)c2cccc3c4ccccc4n(-c4ccc(-c5cccc6ccccc56)cc4)c23)cc1] 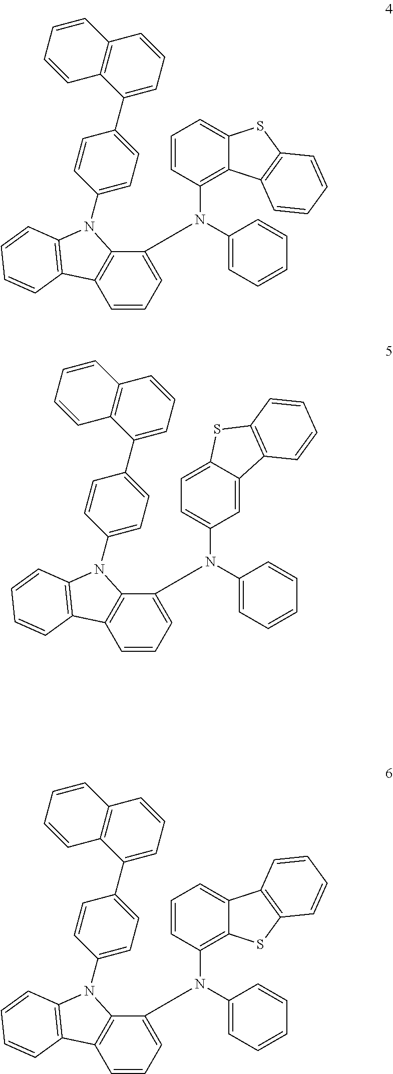 [SMILES: c1ccc(N(c2cccc3sc4ccccc4c23)c2cccc3c4ccccc4n(-c4ccc(-c5cccc6ccccc56)cc4)c23)cc1 ; c1ccc(N(c2ccc3sc4ccccc4c3c2)c2cccc3c4ccccc4n(-c4ccc(-c5cccc6ccccc56)cc4)c23)cc1 ; c1ccc(N(c2cccc3c2sc2ccccc23)c2cccc3c4ccccc4n(-c4ccc(-c5cccc6ccccc56)cc4)c23)cc1] 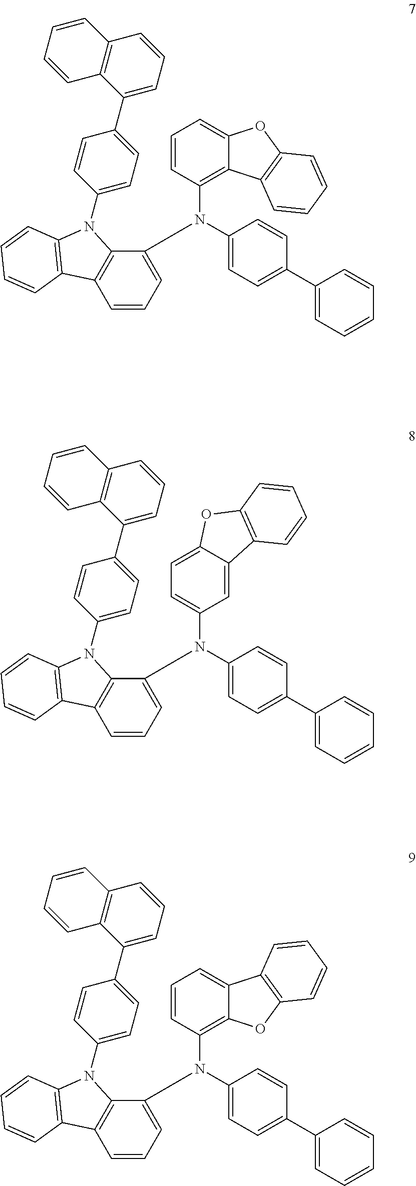 [SMILES: c1ccc(-c2ccc(N(c3cccc4oc5ccccc5c34)c3cccc4c5ccccc5n(-c5ccc(-c6cccc7ccccc67)cc5)c34)cc2)cc1 ; c1ccc(-c2ccc(N(c3ccc4oc5ccccc5c4c3)c3cccc4c5ccccc5n(-c5ccc(-c6cccc7ccccc67)cc5)c34)cc2)cc1 ; c1ccc(-c2ccc(N(c3cccc4c3oc3ccccc34)c3cccc4c5ccccc5n(-c5ccc(-c6cccc7ccccc67)cc5)c34)cc2)cc1] 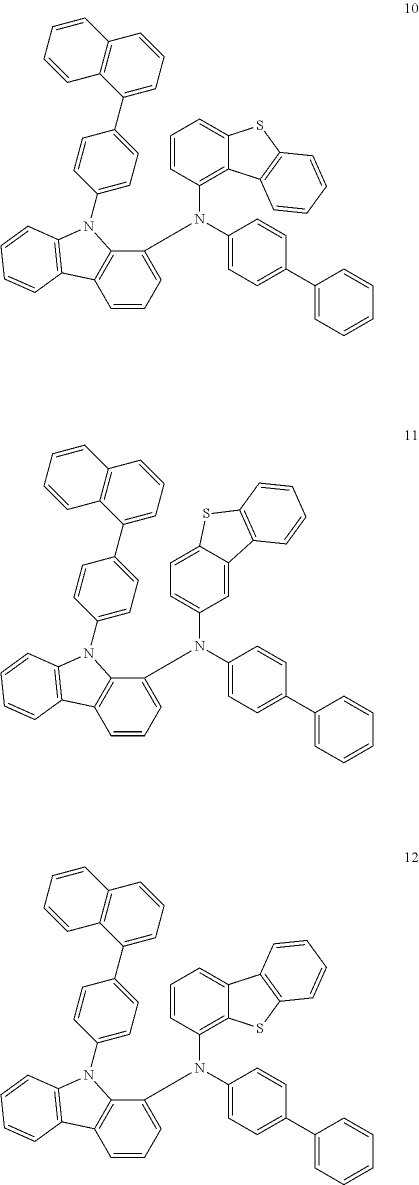 [SMILES: c1ccc(-c2ccc(N(c3cccc4sc5ccccc5c34)c3cccc4c5ccccc5n(-c5ccc(-c6cccc7ccccc67)cc5)c34)cc2)cc1 ; c1ccc(-c2ccc(N(c3ccc4sc5ccccc5c4c3)c3cccc4c5ccccc5n(-c5ccc(-c6cccc7ccccc67)cc5)c34)cc2)cc1 ; c1ccc(-c2ccc(N(c3cccc4c3sc3ccccc34)c3cccc4c5ccccc5n(-c5ccc(-c6cccc7ccccc67)cc5)c34)cc2)cc1] 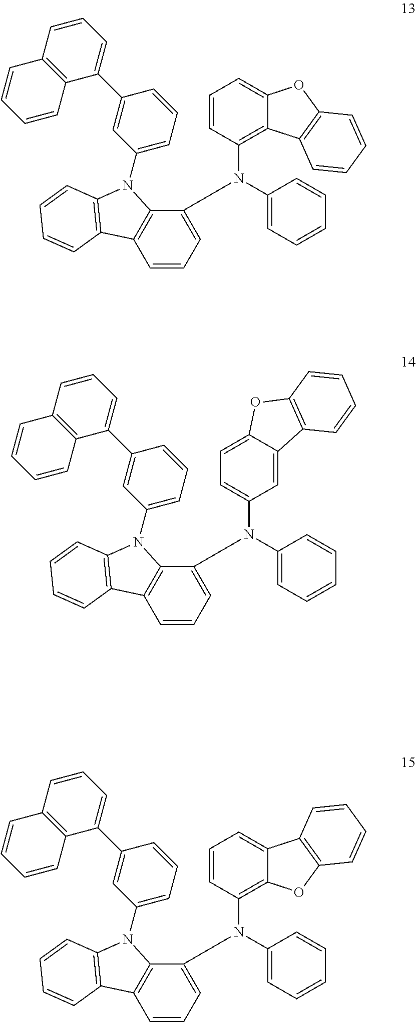 [SMILES: c1ccc(N(c2cccc3oc4ccccc4c23)c2cccc3c4ccccc4n(-c4cccc(-c5cccc6ccccc56)c4)c23)cc1 ; c1ccc(N(c2ccc3oc4ccccc4c3c2)c2cccc3c4ccccc4n(-c4cccc(-c5cccc6ccccc56)c4)c23)cc1 ; c1ccc(N(c2cccc3c2oc2ccccc23)c2cccc3c4ccccc4n(-c4cccc(-c5cccc6ccccc56)c4)c23)cc1] 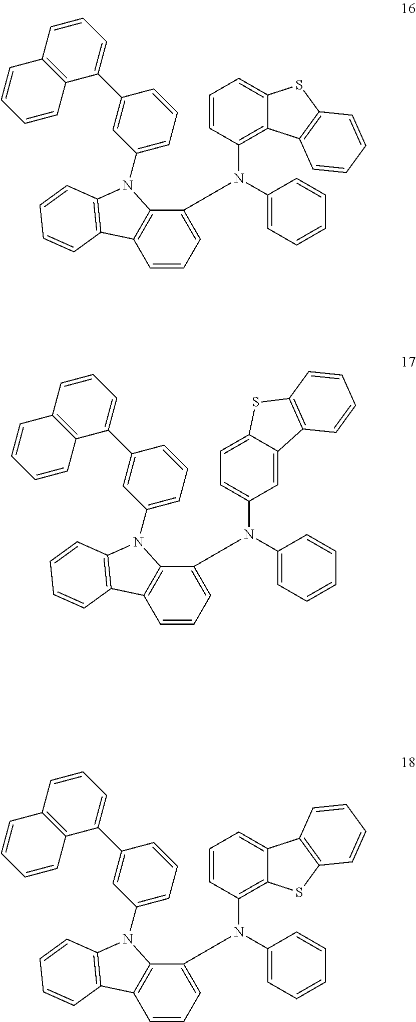 [SMILES: c1ccc(N(c2cccc3sc4ccccc4c23)c2cccc3c4ccccc4n(-c4cccc(-c5cccc6ccccc56)c4)c23)cc1 ; c1ccc(N(c2ccc3sc4ccccc4c3c2)c2cccc3c4ccccc4n(-c4cccc(-c5cccc6ccccc56)c4)c23)cc1 ; c1ccc(N(c2cccc3c2sc2ccccc23)c2cccc3c4ccccc4n(-c4cccc(-c5cccc6ccccc56)c4)c23)cc1] 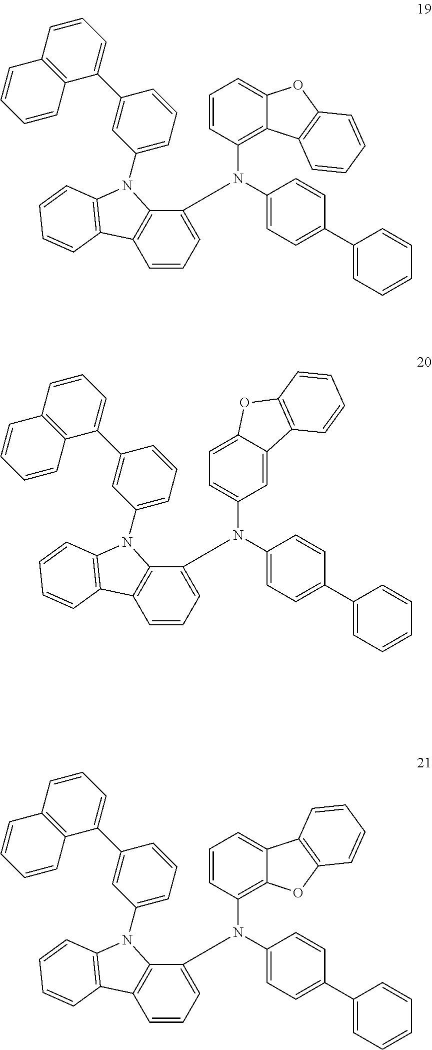 [SMILES: c1ccc(-c2ccc(N(c3cccc4oc5ccccc5c34)c3cccc4c5ccccc5n(-c5cccc(-c6cccc7ccccc67)c5)c34)cc2)cc1 ; c1ccc(-c2ccc(N(c3ccc4oc5ccccc5c4c3)c3cccc4c5ccccc5n(-c5cccc(-c6cccc7ccccc67)c5)c34)cc2)cc1 ; c1ccc(-c2ccc(N(c3cccc4c3oc3ccccc34)c3cccc4c5ccccc5n(-c5cccc(-c6cccc7ccccc67)c5)c34)cc2)cc1] 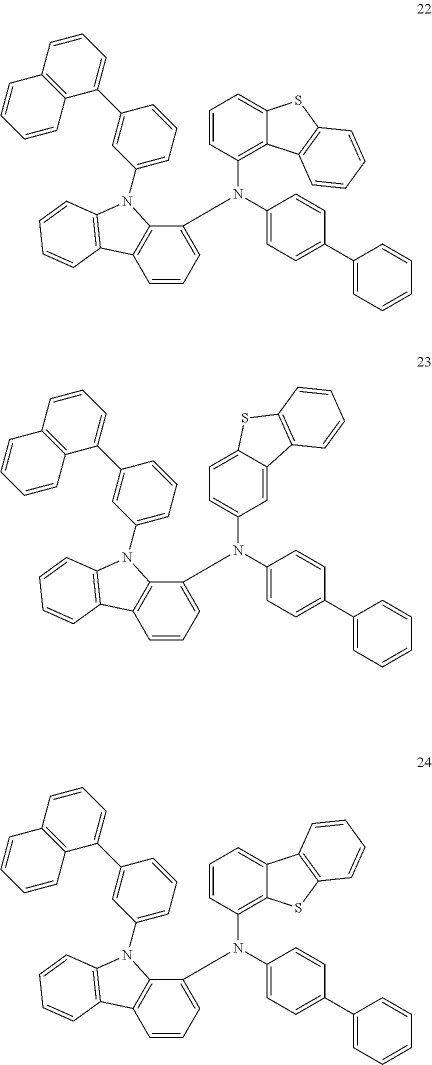 [SMILES: c1ccc(-c2ccc(N(c3cccc4sc5ccccc5c34)c3cccc4c5ccccc5n(-c5cccc(-c6cccc7ccccc67)c5)c34)cc2)cc1 ; c1ccc(-c2ccc(N(c3ccc4sc5ccccc5c4c3)c3cccc4c5ccccc5n(-c5cccc(-c6cccc7ccccc67)c5)c34)cc2)cc1 ; c1ccc(-c2ccc(N(c3cccc4c3sc3ccccc34)c3cccc4c5ccccc5n(-c5cccc(-c6cccc7ccccc67)c5)c34)cc2)cc1] 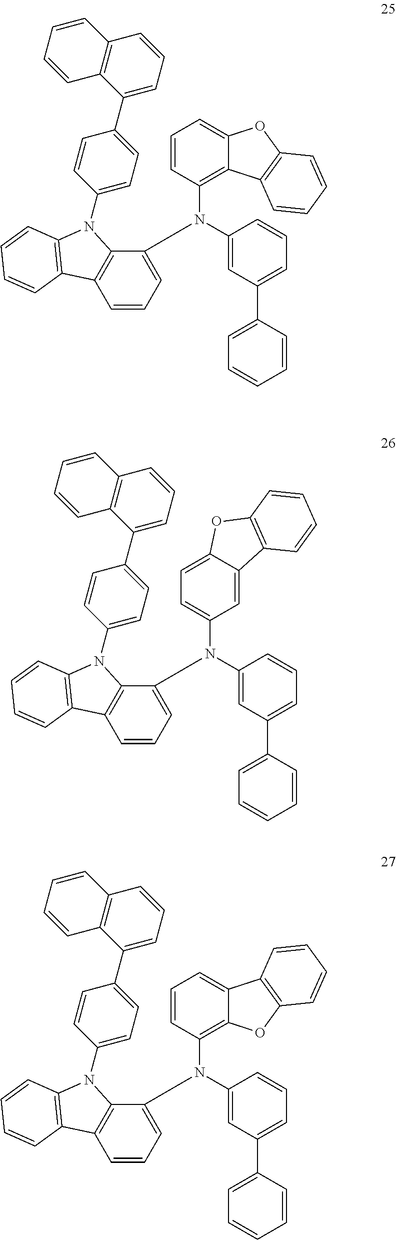 [SMILES: c1ccc(-c2cccc(N(c3cccc4oc5ccccc5c34)c3cccc4c5ccccc5n(-c5ccc(-c6cccc7ccccc67)cc5)c34)c2)cc1 ; c1ccc(-c2cccc(N(c3ccc4oc5ccccc5c4c3)c3cccc4c5ccccc5n(-c5ccc(-c6cccc7ccccc67)cc5)c34)c2)cc1 ; c1ccc(-c2cccc(N(c3cccc4c3oc3ccccc34)c3cccc4c5ccccc5n(-c5ccc(-c6cccc7ccccc67)cc5)c34)c2)cc1] 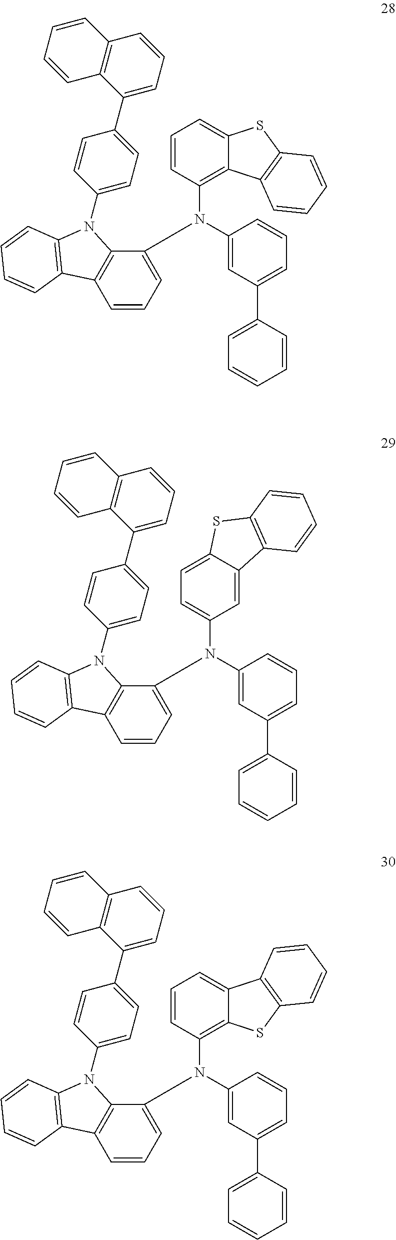 [SMILES: c1ccc(-c2cccc(N(c3cccc4sc5ccccc5c34)c3cccc4c5ccccc5n(-c5ccc(-c6cccc7ccccc67)cc5)c34)c2)cc1 ; c1ccc(-c2cccc(N(c3ccc4sc5ccccc5c4c3)c3cccc4c5ccccc5n(-c5ccc(-c6cccc7ccccc67)cc5)c34)c2)cc1 ; c1ccc(-c2cccc(N(c3cccc4c3sc3ccccc34)c3cccc4c5ccccc5n(-c5ccc(-c6cccc7ccccc67)cc5)c34)c2)cc1] 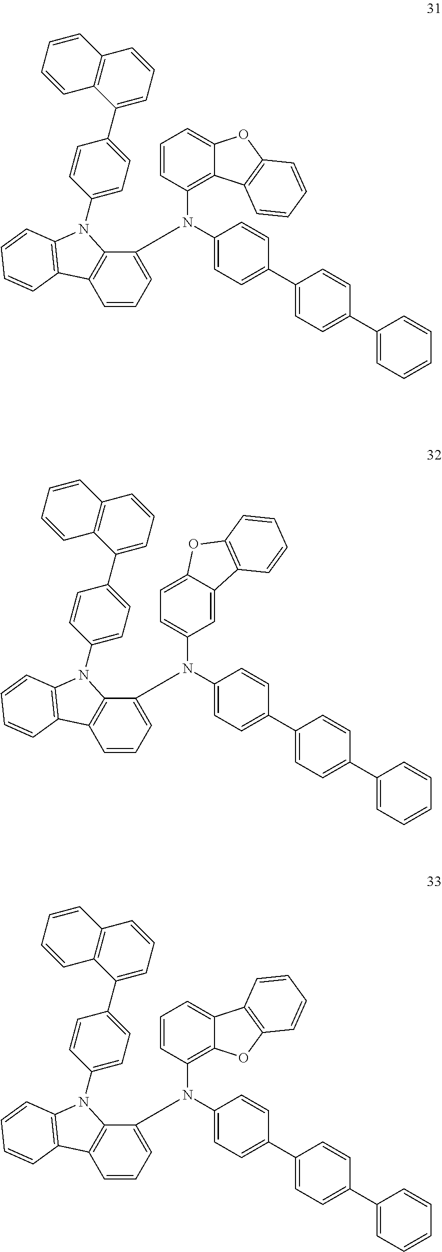 [SMILES: c1ccc(-c2ccc(-c3ccc(N(c4cccc5oc6ccccc6c45)c4cccc5c6ccccc6n(-c6ccc(-c7cccc8ccccc78)cc6)c45)cc3)cc2)cc1 ; c1ccc(-c2ccc(-c3ccc(N(c4ccc5oc6ccccc6c5c4)c4cccc5c6ccccc6n(-c6ccc(-c7cccc8ccccc78)cc6)c45)cc3)cc2)cc1 ; c1ccc(-c2ccc(-c3ccc(N(c4cccc5c4oc4ccccc45)c4cccc5c6ccccc6n(-c6ccc(-c7cccc8ccccc78)cc6)c45)cc3)cc2)cc1] 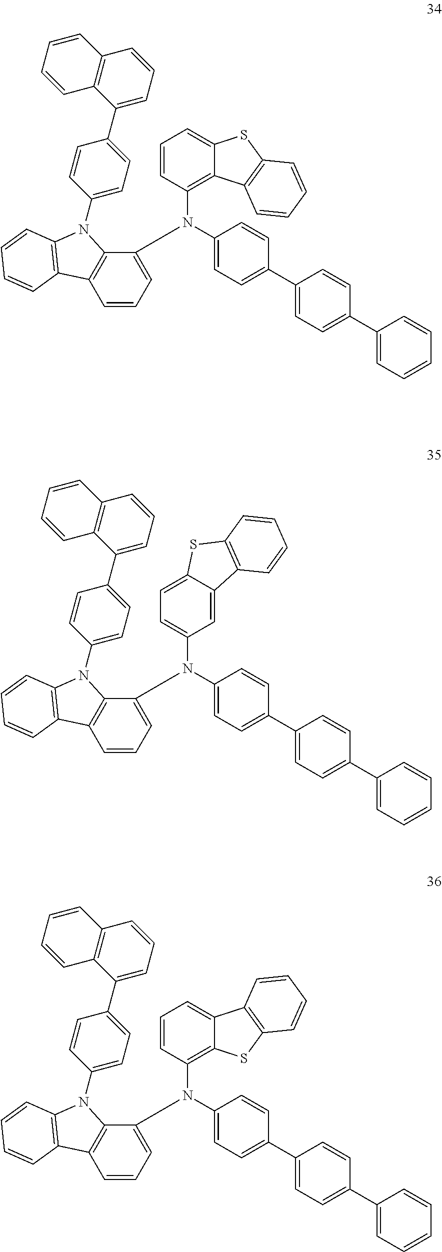 [SMILES: c1ccc(-c2ccc(-c3ccc(N(c4cccc5sc6ccccc6c45)c4cccc5c6ccccc6n(-c6ccc(-c7cccc8ccccc78)cc6)c45)cc3)cc2)cc1 ; c1ccc(-c2ccc(-c3ccc(N(c4ccc5sc6ccccc6c5c4)c4cccc5c6ccccc6n(-c6ccc(-c7cccc8ccccc78)cc6)c45)cc3)cc2)cc1 ; c1ccc(-c2ccc(-c3ccc(N(c4cccc5c4sc4ccccc45)c4cccc5c6ccccc6n(-c6ccc(-c7cccc8ccccc78)cc6)c45)cc3)cc2)cc1] 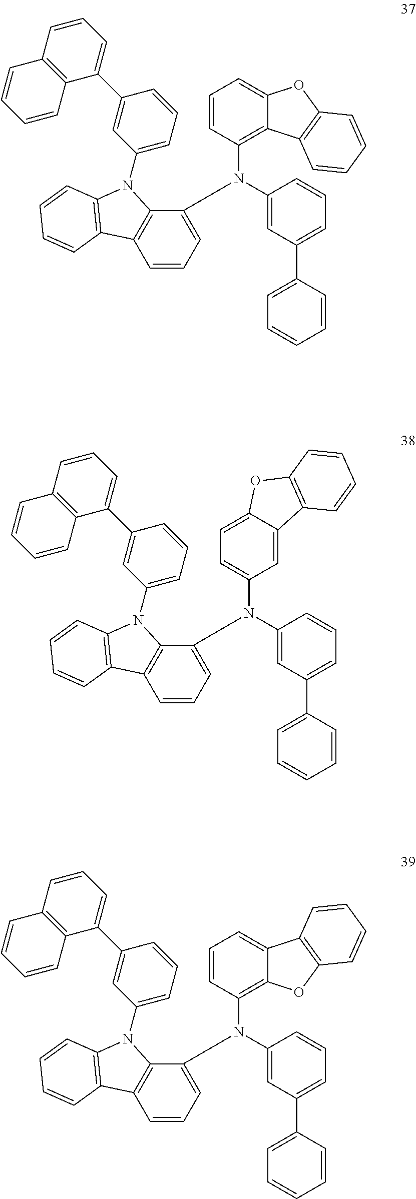 [SMILES: c1ccc(-c2cccc(N(c3cccc4oc5ccccc5c34)c3cccc4c5ccccc5n(-c5cccc(-c6cccc7ccccc67)c5)c34)c2)cc1 ; c1ccc(-c2cccc(N(c3ccc4oc5ccccc5c4c3)c3cccc4c5ccccc5n(-c5cccc(-c6cccc7ccccc67)c5)c34)c2)cc1 ; c1ccc(-c2cccc(N(c3cccc4c3oc3ccccc34)c3cccc4c5ccccc5n(-c5cccc(-c6cccc7ccccc67)c5)c34)c2)cc1] 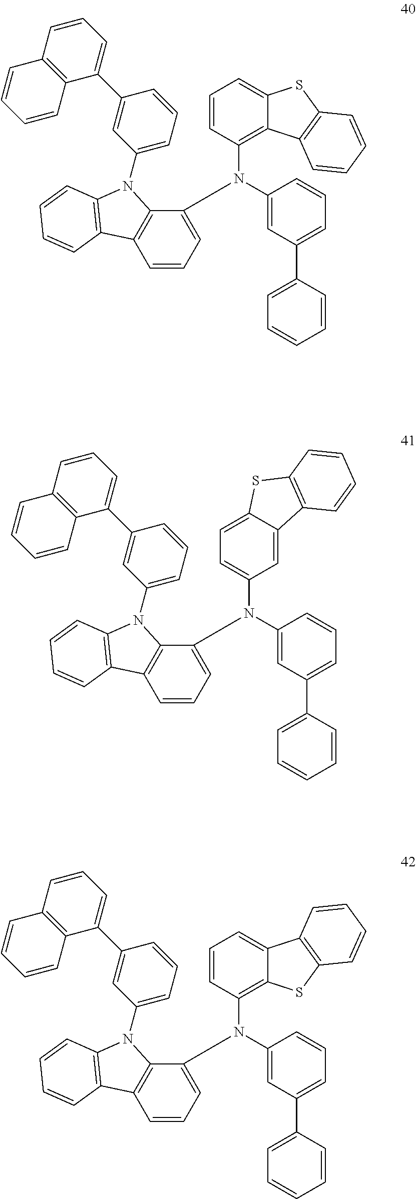 [SMILES: c1ccc(-c2cccc(N(c3cccc4sc5ccccc5c34)c3cccc4c5ccccc5n(-c5cccc(-c6cccc7ccccc67)c5)c34)c2)cc1 ; c1ccc(-c2cccc(N(c3ccc4sc5ccccc5c4c3)c3cccc4c5ccccc5n(-c5cccc(-c6cccc7ccccc67)c5)c34)c2)cc1 ; c1ccc(-c2cccc(N(c3cccc4c3sc3ccccc34)c3cccc4c5ccccc5n(-c5cccc(-c6cccc7ccccc67)c5)c34)c2)cc1] 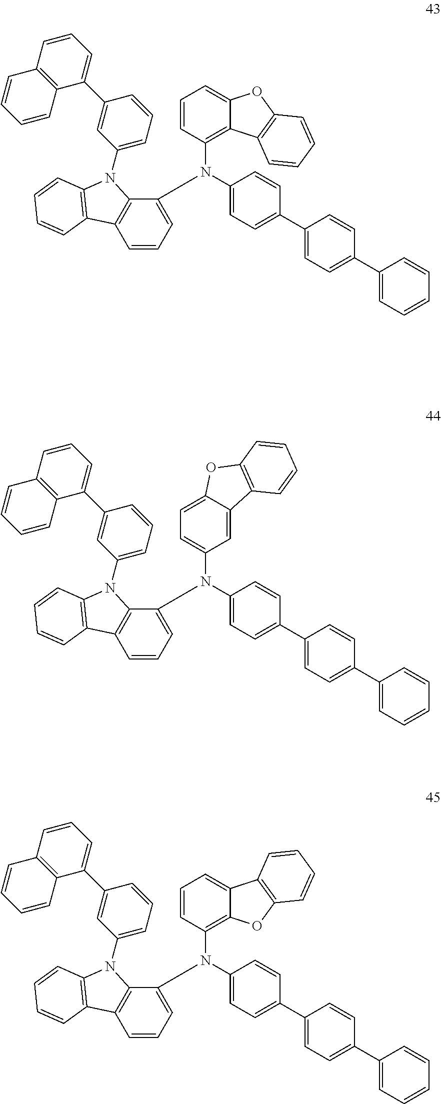 [SMILES: c1ccc(-c2ccc(-c3ccc(N(c4cccc5oc6ccccc6c45)c4cccc5c6ccccc6n(-c6cccc(-c7cccc8ccccc78)c6)c45)cc3)cc2)cc1 ; c1ccc(-c2ccc(-c3ccc(N(c4ccc5oc6ccccc6c5c4)c4cccc5c6ccccc6n(-c6cccc(-c7cccc8ccccc78)c6)c45)cc3)cc2)cc1 ; c1ccc(-c2ccc(-c3ccc(N(c4cccc5c4oc4ccccc45)c4cccc5c6ccccc6n(-c6cccc(-c7cccc8ccccc78)c6)c45)cc3)cc2)cc1] 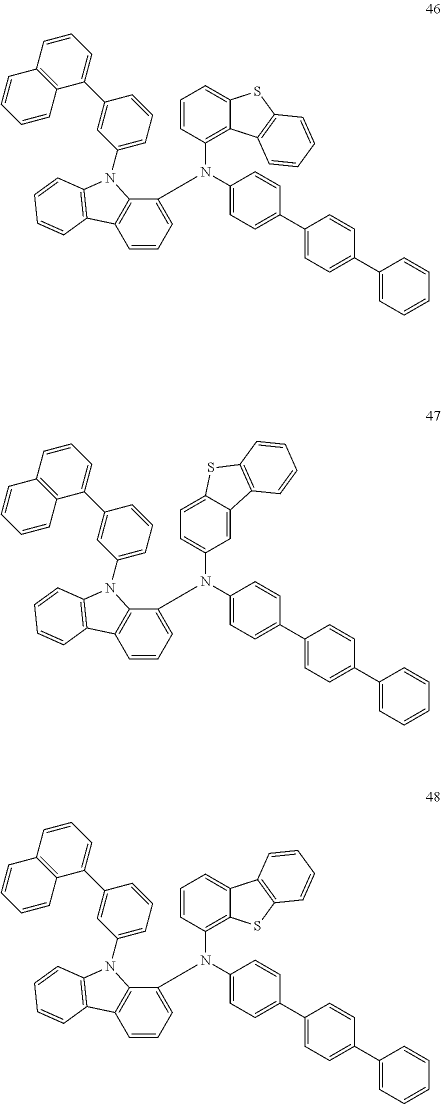 [SMILES: c1ccc(-c2ccc(-c3ccc(N(c4cccc5sc6ccccc6c45)c4cccc5c6ccccc6n(-c6cccc(-c7cccc8ccccc78)c6)c45)cc3)cc2)cc1 ; c1ccc(-c2ccc(-c3ccc(N(c4ccc5sc6ccccc6c5c4)c4cccc5c6ccccc6n(-c6cccc(-c7cccc8ccccc78)c6)c45)cc3)cc2)cc1 ; c1ccc(-c2ccc(-c3ccc(N(c4cccc5c4sc4ccccc45)c4cccc5c6ccccc6n(-c6cccc(-c7cccc8ccccc78)c6)c45)cc3)cc2)cc1] 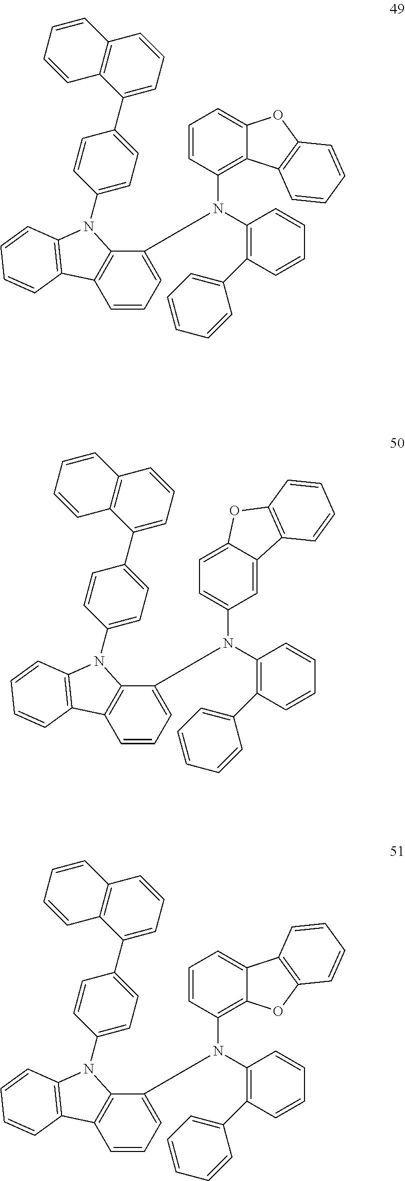 [SMILES: c1ccc(-c2ccccc2N(c2cccc3oc4ccccc4c23)c2cccc3c4ccccc4n(-c4ccc(-c5cccc6ccccc56)cc4)c23)cc1 ; c1ccc(-c2ccccc2N(c2ccc3oc4ccccc4c3c2)c2cccc3c4ccccc4n(-c4ccc(-c5cccc6ccccc56)cc4)c23)cc1 ; c1ccc(-c2ccccc2N(c2cccc3c2oc2ccccc23)c2cccc3c4ccccc4n(-c4ccc(-c5cccc6ccccc56)cc4)c23)cc1] 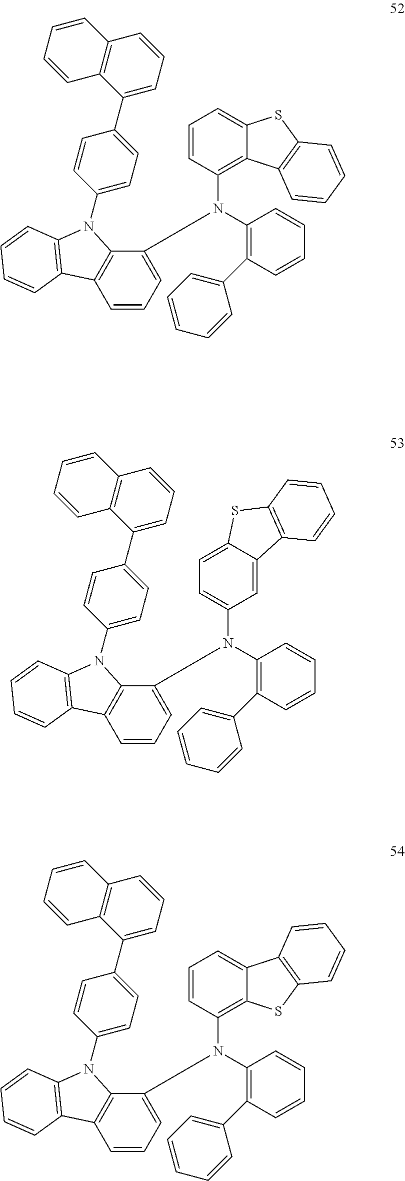 [SMILES: c1ccc(-c2ccccc2N(c2cccc3sc4ccccc4c23)c2cccc3c4ccccc4n(-c4ccc(-c5cccc6ccccc56)cc4)c23)cc1 ; c1ccc(-c2ccccc2N(c2ccc3sc4ccccc4c3c2)c2cccc3c4ccccc4n(-c4ccc(-c5cccc6ccccc56)cc4)c23)cc1 ; c1ccc(-c2ccccc2N(c2cccc3c2sc2ccccc23)c2cccc3c4ccccc4n(-c4ccc(-c5cccc6ccccc56)cc4)c23)cc1] 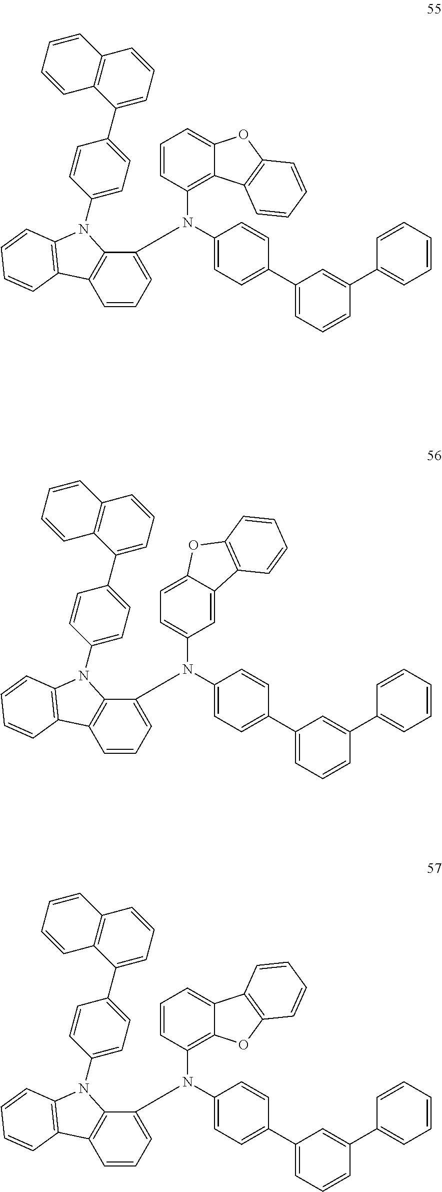 [SMILES: c1ccc(-c2cccc(-c3ccc(N(c4cccc5oc6ccccc6c45)c4cccc5c6ccccc6n(-c6ccc(-c7cccc8ccccc78)cc6)c45)cc3)c2)cc1 ; c1ccc(-c2cccc(-c3ccc(N(c4ccc5oc6ccccc6c5c4)c4cccc5c6ccccc6n(-c6ccc(-c7cccc8ccccc78)cc6)c45)cc3)c2)cc1 ; c1ccc(-c2cccc(-c3ccc(N(c4cccc5c4oc4ccccc45)c4cccc5c6ccccc6n(-c6ccc(-c7cccc8ccccc78)cc6)c45)cc3)c2)cc1] 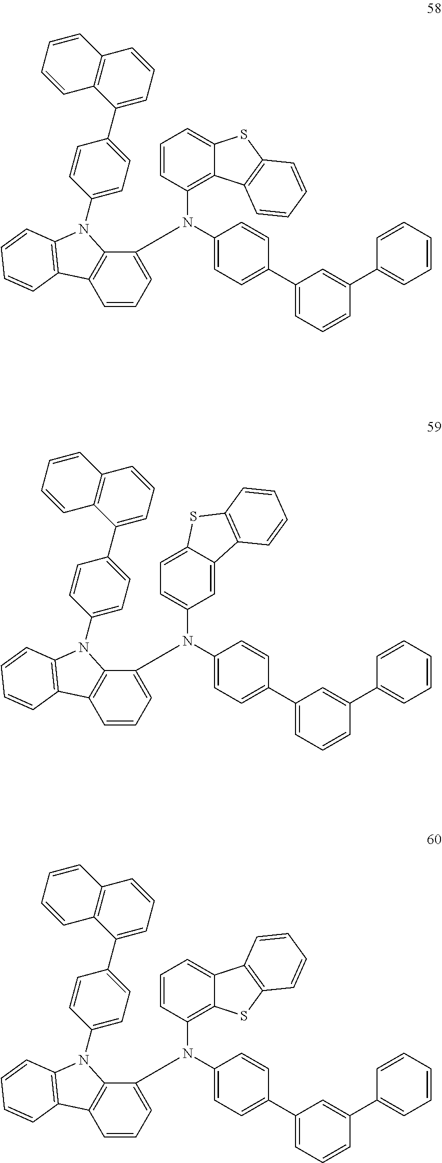 [SMILES: c1ccc(-c2cccc(-c3ccc(N(c4cccc5sc6ccccc6c45)c4cccc5c6ccccc6n(-c6ccc(-c7cccc8ccccc78)cc6)c45)cc3)c2)cc1 ; c1ccc(-c2cccc(-c3ccc(N(c4ccc5sc6ccccc6c5c4)c4cccc5c6ccccc6n(-c6ccc(-c7cccc8ccccc78)cc6)c45)cc3)c2)cc1 ; c1ccc(-c2cccc(-c3ccc(N(c4cccc5c4sc4ccccc45)c4cccc5c6ccccc6n(-c6ccc(-c7cccc8ccccc78)cc6)c45)cc3)c2)cc1] 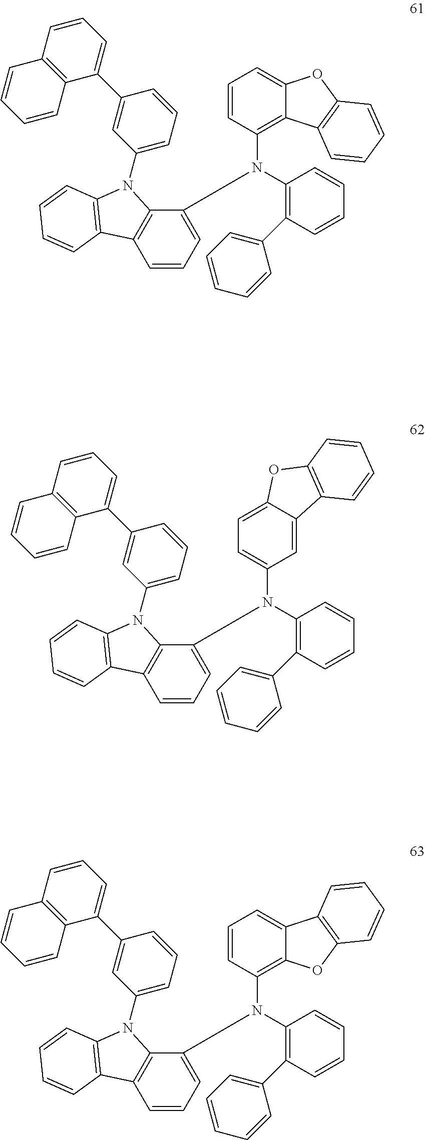 [SMILES: c1ccc(-c2ccccc2N(c2cccc3oc4ccccc4c23)c2cccc3c4ccccc4n(-c4cccc(-c5cccc6ccccc56)c4)c23)cc1 ; c1ccc(-c2ccccc2N(c2ccc3oc4ccccc4c3c2)c2cccc3c4ccccc4n(-c4cccc(-c5cccc6ccccc56)c4)c23)cc1 ; c1ccc(-c2ccccc2N(c2cccc3c2oc2ccccc23)c2cccc3c4ccccc4n(-c4cccc(-c5cccc6ccccc56)c4)c23)cc1] 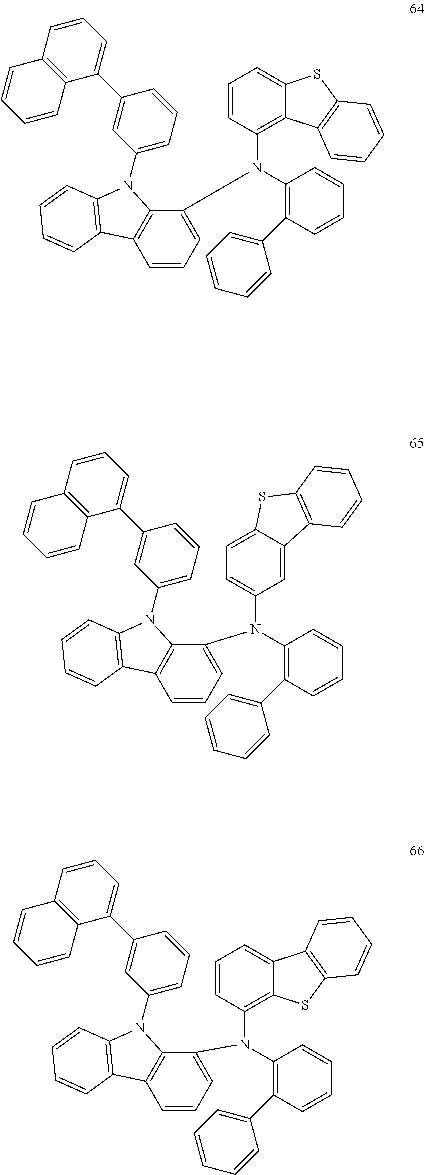 [SMILES: c1ccc(-c2ccccc2N(c2cccc3sc4ccccc4c23)c2cccc3c4ccccc4n(-c4cccc(-c5cccc6ccccc56)c4)c23)cc1 ; c1ccc(-c2ccccc2N(c2ccc3sc4ccccc4c3c2)c2cccc3c4ccccc4n(-c4cccc(-c5cccc6ccccc56)c4)c23)cc1 ; c1ccc(-c2ccccc2N(c2cccc3c2sc2ccccc23)c2cccc3c4ccccc4n(-c4cccc(-c5cccc6ccccc56)c4)c23)cc1] 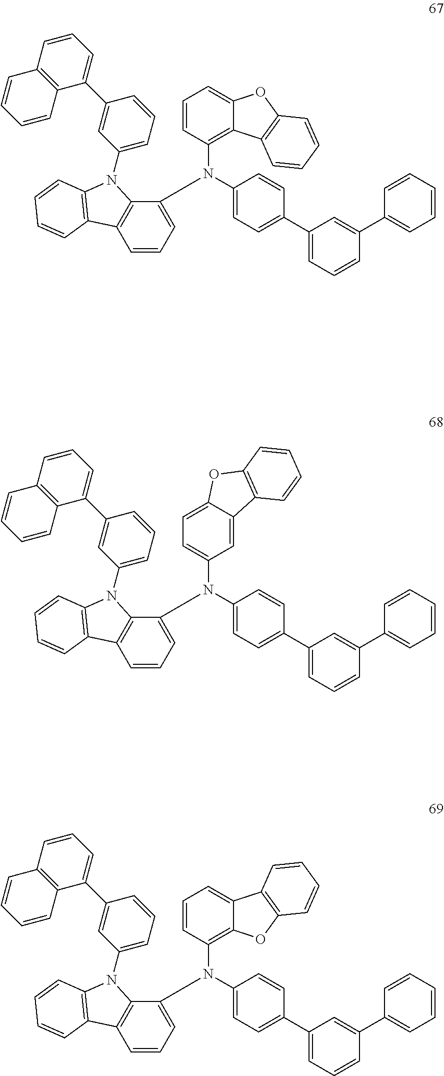 [SMILES: c1ccc(-c2cccc(-c3ccc(N(c4cccc5oc6ccccc6c45)c4cccc5c6ccccc6n(-c6cccc(-c7cccc8ccccc78)c6)c45)cc3)c2)cc1 ; c1ccc(-c2cccc(-c3ccc(N(c4ccc5oc6ccccc6c5c4)c4cccc5c6ccccc6n(-c6cccc(-c7cccc8ccccc78)c6)c45)cc3)c2)cc1 ; c1ccc(-c2cccc(-c3ccc(N(c4cccc5c4oc4ccccc45)c4cccc5c6ccccc6n(-c6cccc(-c7cccc8ccccc78)c6)c45)cc3)c2)cc1] 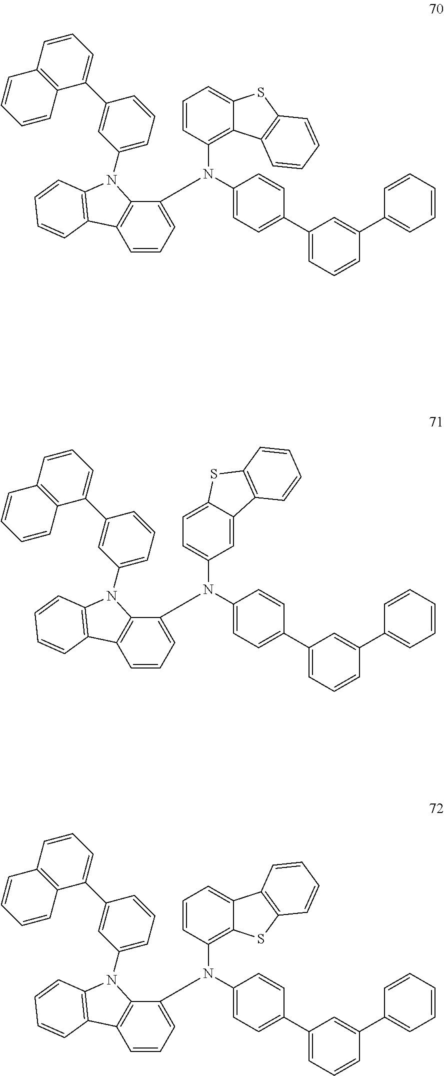 [SMILES: c1ccc(-c2cccc(-c3ccc(N(c4cccc5sc6ccccc6c45)c4cccc5c6ccccc6n(-c6cccc(-c7cccc8ccccc78)c6)c45)cc3)c2)cc1 ; c1ccc(-c2cccc(-c3ccc(N(c4ccc5sc6ccccc6c5c4)c4cccc5c6ccccc6n(-c6cccc(-c7cccc8ccccc78)c6)c45)cc3)c2)cc1 ; c1ccc(-c2cccc(-c3ccc(N(c4cccc5c4sc4ccccc45)c4cccc5c6ccccc6n(-c6cccc(-c7cccc8ccccc78)c6)c45)cc3)c2)cc1] 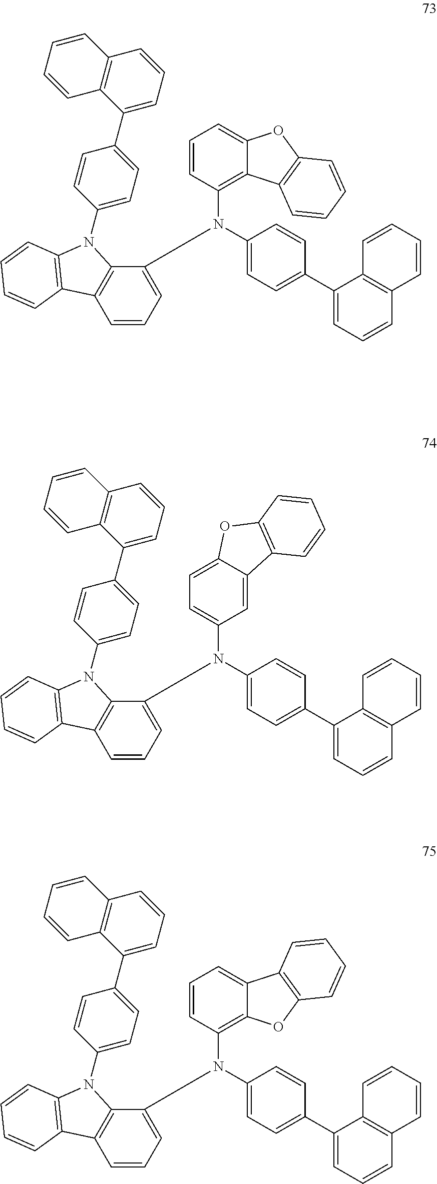 [SMILES: c1ccc2c(-c3ccc(N(c4cccc5oc6ccccc6c45)c4cccc5c6ccccc6n(-c6ccc(-c7cccc8ccccc78)cc6)c45)cc3)cccc2c1 ; c1ccc2c(-c3ccc(N(c4ccc5oc6ccccc6c5c4)c4cccc5c6ccccc6n(-c6ccc(-c7cccc8ccccc78)cc6)c45)cc3)cccc2c1 ; c1ccc2c(-c3ccc(N(c4cccc5c4oc4ccccc45)c4cccc5c6ccccc6n(-c6ccc(-c7cccc8ccccc78)cc6)c45)cc3)cccc2c1] 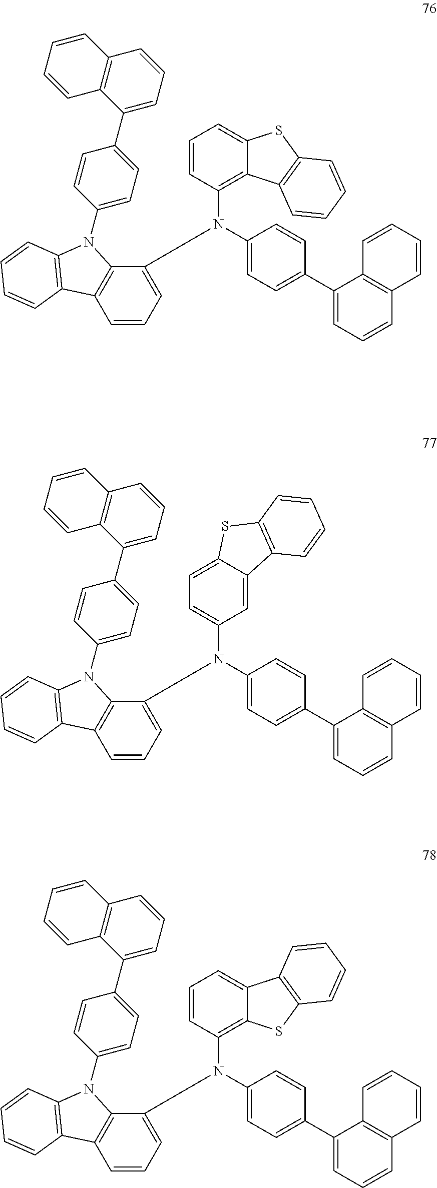 [SMILES: c1ccc2c(-c3ccc(N(c4cccc5sc6ccccc6c45)c4cccc5c6ccccc6n(-c6ccc(-c7cccc8ccccc78)cc6)c45)cc3)cccc2c1 ; c1ccc2c(-c3ccc(N(c4ccc5sc6ccccc6c5c4)c4cccc5c6ccccc6n(-c6ccc(-c7cccc8ccccc78)cc6)c45)cc3)cccc2c1 ; c1ccc2c(-c3ccc(N(c4cccc5c4sc4ccccc45)c4cccc5c6ccccc6n(-c6ccc(-c7cccc8ccccc78)cc6)c45)cc3)cccc2c1] 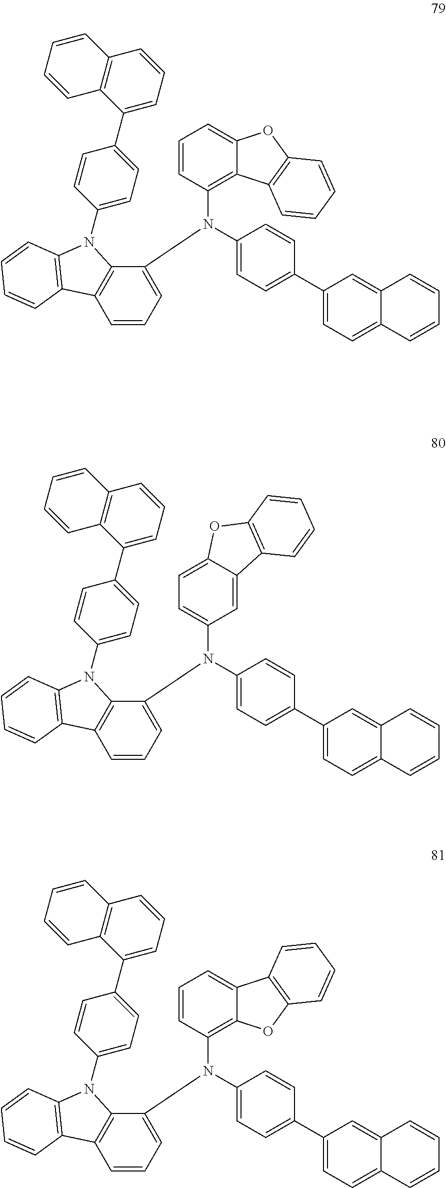 [SMILES: c1ccc2cc(-c3ccc(N(c4cccc5oc6ccccc6c45)c4cccc5c6ccccc6n(-c6ccc(-c7cccc8ccccc78)cc6)c45)cc3)ccc2c1 ; c1ccc2cc(-c3ccc(N(c4ccc5oc6ccccc6c5c4)c4cccc5c6ccccc6n(-c6ccc(-c7cccc8ccccc78)cc6)c45)cc3)ccc2c1 ; c1ccc2cc(-c3ccc(N(c4cccc5c4oc4ccccc45)c4cccc5c6ccccc6n(-c6ccc(-c7cccc8ccccc78)cc6)c45)cc3)ccc2c1] 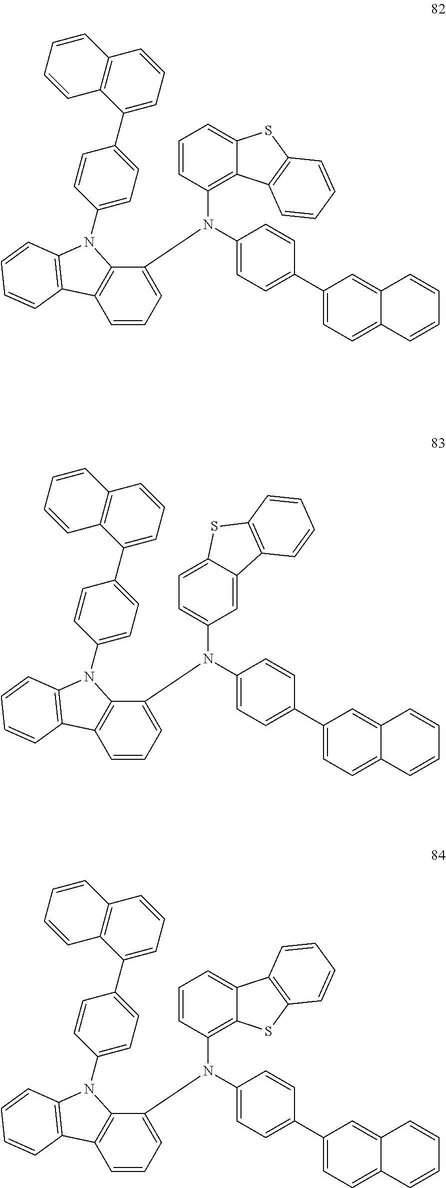 [SMILES: c1ccc2cc(-c3ccc(N(c4cccc5sc6ccccc6c45)c4cccc5c6ccccc6n(-c6ccc(-c7cccc8ccccc78)cc6)c45)cc3)ccc2c1 ; c1ccc2cc(-c3ccc(N(c4ccc5sc6ccccc6c5c4)c4cccc5c6ccccc6n(-c6ccc(-c7cccc8ccccc78)cc6)c45)cc3)ccc2c1 ; c1ccc2cc(-c3ccc(N(c4cccc5c4sc4ccccc45)c4cccc5c6ccccc6n(-c6ccc(-c7cccc8ccccc78)cc6)c45)cc3)ccc2c1] 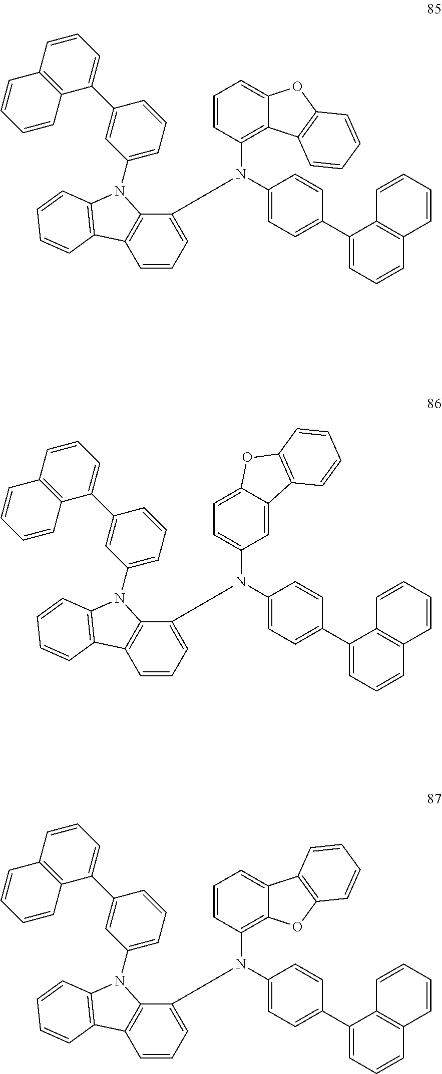 [SMILES: c1cc(-c2cccc3ccccc23)cc(-n2c3ccccc3c3cccc(N(c4ccc(-c5cccc6ccccc56)cc4)c4cccc5oc6ccccc6c45)c32)c1 ; c1cc(-c2cccc3ccccc23)cc(-n2c3ccccc3c3cccc(N(c4ccc(-c5cccc6ccccc56)cc4)c4ccc5oc6ccccc6c5c4)c32)c1 ; c1cc(-c2cccc3ccccc23)cc(-n2c3ccccc3c3cccc(N(c4ccc(-c5cccc6ccccc56)cc4)c4cccc5c4oc4ccccc45)c32)c1] 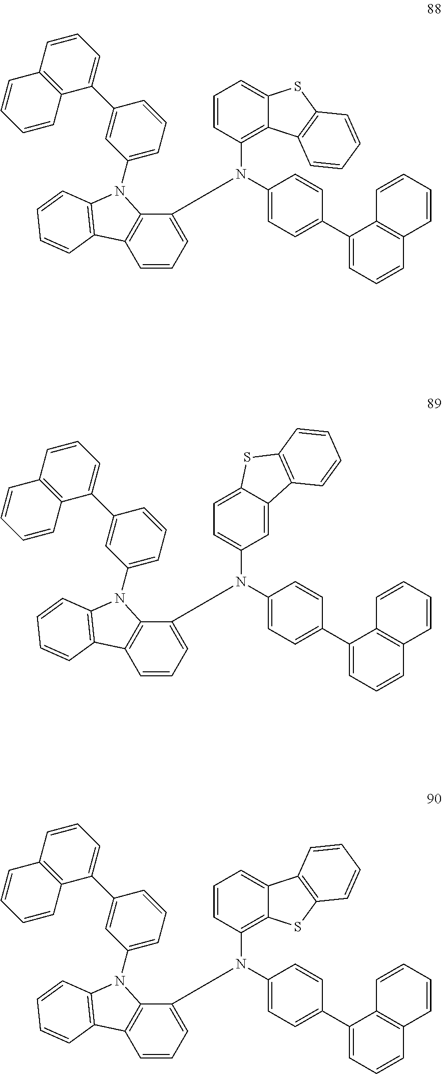 [SMILES: c1cc(-c2cccc3ccccc23)cc(-n2c3ccccc3c3cccc(N(c4ccc(-c5cccc6ccccc56)cc4)c4cccc5sc6ccccc6c45)c32)c1 ; c1cc(-c2cccc3ccccc23)cc(-n2c3ccccc3c3cccc(N(c4ccc(-c5cccc6ccccc56)cc4)c4ccc5sc6ccccc6c5c4)c32)c1 ; c1cc(-c2cccc3ccccc23)cc(-n2c3ccccc3c3cccc(N(c4ccc(-c5cccc6ccccc56)cc4)c4cccc5c4sc4ccccc45)c32)c1] 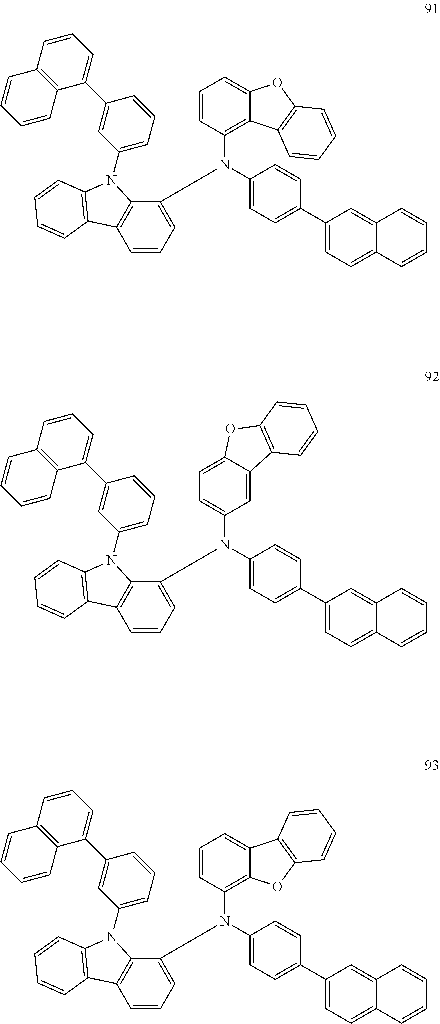 [SMILES: c1cc(-c2cccc3ccccc23)cc(-n2c3ccccc3c3cccc(N(c4ccc(-c5ccc6ccccc6c5)cc4)c4cccc5oc6ccccc6c45)c32)c1 ; c1cc(-c2cccc3ccccc23)cc(-n2c3ccccc3c3cccc(N(c4ccc(-c5ccc6ccccc6c5)cc4)c4ccc5oc6ccccc6c5c4)c32)c1 ; c1cc(-c2cccc3ccccc23)cc(-n2c3ccccc3c3cccc(N(c4ccc(-c5ccc6ccccc6c5)cc4)c4cccc5c4oc4ccccc45)c32)c1] 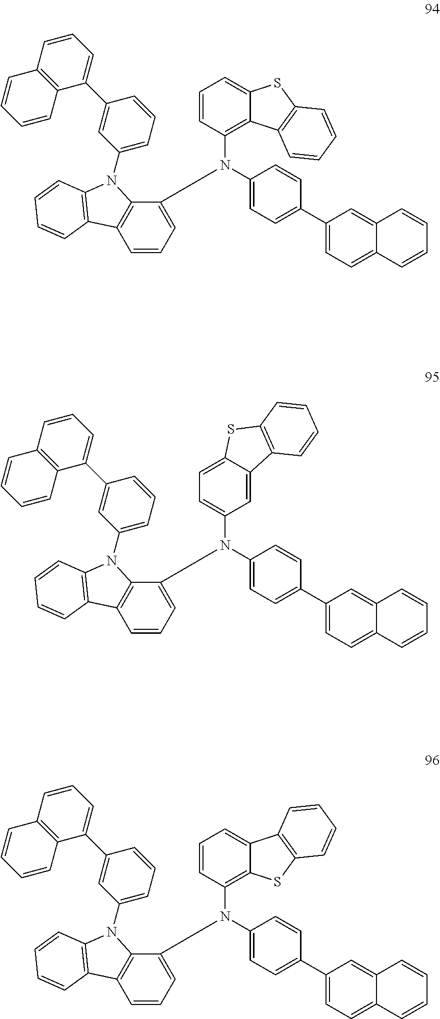 [SMILES: c1cc(-c2cccc3ccccc23)cc(-n2c3ccccc3c3cccc(N(c4ccc(-c5ccc6ccccc6c5)cc4)c4cccc5sc6ccccc6c45)c32)c1 ; c1cc(-c2cccc3ccccc23)cc(-n2c3ccccc3c3cccc(N(c4ccc(-c5ccc6ccccc6c5)cc4)c4ccc5sc6ccccc6c5c4)c32)c1 ; c1cc(-c2cccc3ccccc23)cc(-n2c3ccccc3c3cccc(N(c4ccc(-c5ccc6ccccc6c5)cc4)c4cccc5c4sc4ccccc45)c32)c1] 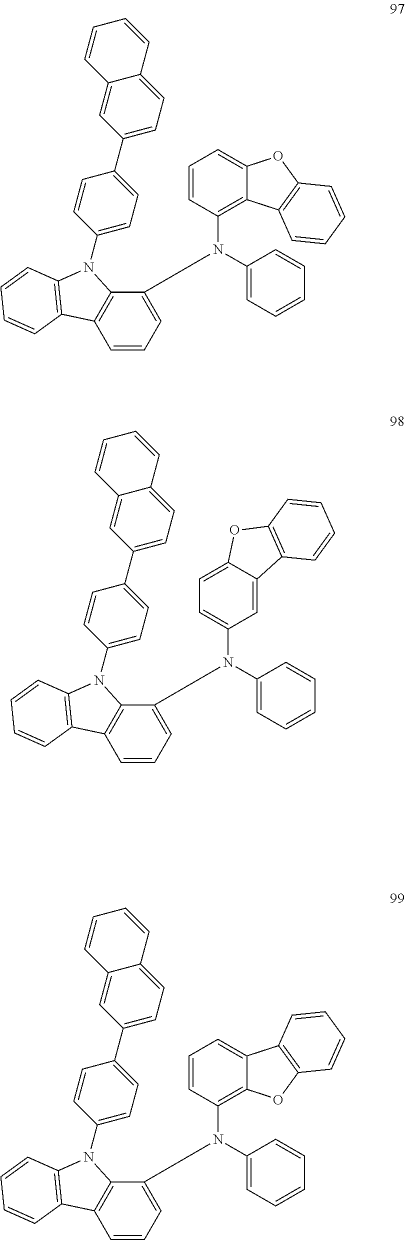 [SMILES: c1ccc(N(c2cccc3oc4ccccc4c23)c2cccc3c4ccccc4n(-c4ccc(-c5ccc6ccccc6c5)cc4)c23)cc1 ; c1ccc(N(c2ccc3oc4ccccc4c3c2)c2cccc3c4ccccc4n(-c4ccc(-c5ccc6ccccc6c5)cc4)c23)cc1 ; c1ccc(N(c2cccc3c2oc2ccccc23)c2cccc3c4ccccc4n(-c4ccc(-c5ccc6ccccc6c5)cc4)c23)cc1] 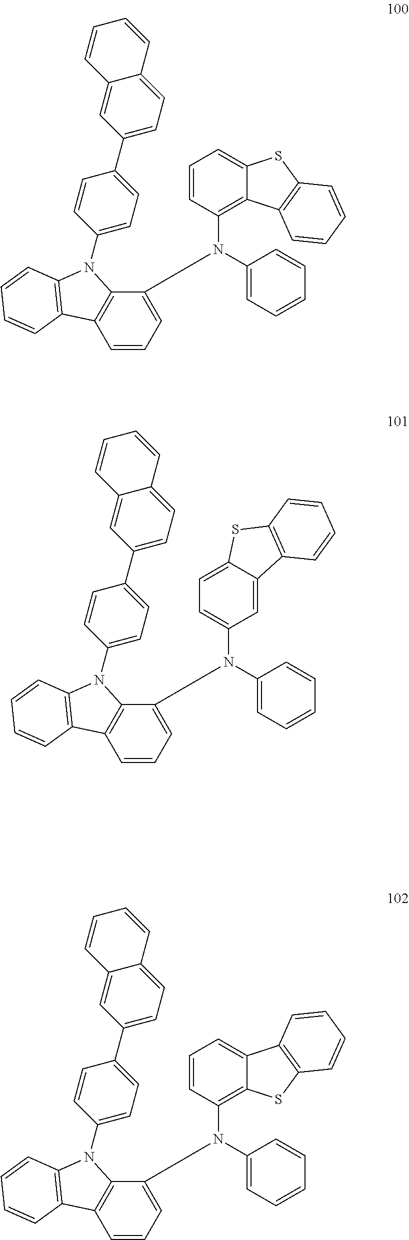 [SMILES: c1ccc(N(c2cccc3sc4ccccc4c23)c2cccc3c4ccccc4n(-c4ccc(-c5ccc6ccccc6c5)cc4)c23)cc1 ; c1ccc(N(c2ccc3sc4ccccc4c3c2)c2cccc3c4ccccc4n(-c4ccc(-c5ccc6ccccc6c5)cc4)c23)cc1 ; c1ccc(N(c2cccc3c2sc2ccccc23)c2cccc3c4ccccc4n(-c4ccc(-c5ccc6ccccc6c5)cc4)c23)cc1] 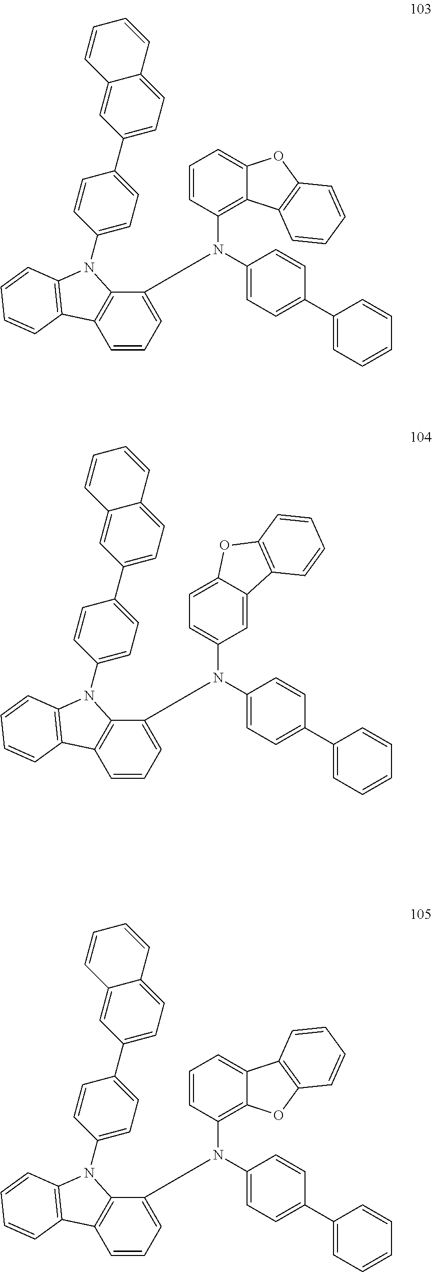 [SMILES: c1ccc(-c2ccc(N(c3cccc4oc5ccccc5c34)c3cccc4c5ccccc5n(-c5ccc(-c6ccc7ccccc7c6)cc5)c34)cc2)cc1 ; c1ccc(-c2ccc(N(c3ccc4oc5ccccc5c4c3)c3cccc4c5ccccc5n(-c5ccc(-c6ccc7ccccc7c6)cc5)c34)cc2)cc1 ; c1ccc(-c2ccc(N(c3cccc4c3oc3ccccc34)c3cccc4c5ccccc5n(-c5ccc(-c6ccc7ccccc7c6)cc5)c34)cc2)cc1] 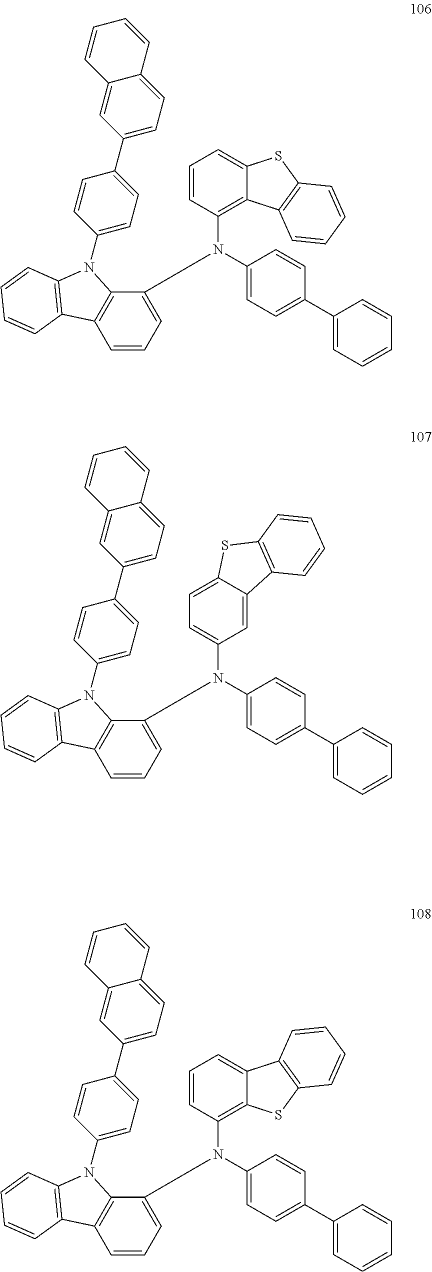 [SMILES: c1ccc(-c2ccc(N(c3cccc4sc5ccccc5c34)c3cccc4c5ccccc5n(-c5ccc(-c6ccc7ccccc7c6)cc5)c34)cc2)cc1 ; c1ccc(-c2ccc(N(c3ccc4sc5ccccc5c4c3)c3cccc4c5ccccc5n(-c5ccc(-c6ccc7ccccc7c6)cc5)c34)cc2)cc1 ; c1ccc(-c2ccc(N(c3cccc4c3sc3ccccc34)c3cccc4c5ccccc5n(-c5ccc(-c6ccc7ccccc7c6)cc5)c34)cc2)cc1] 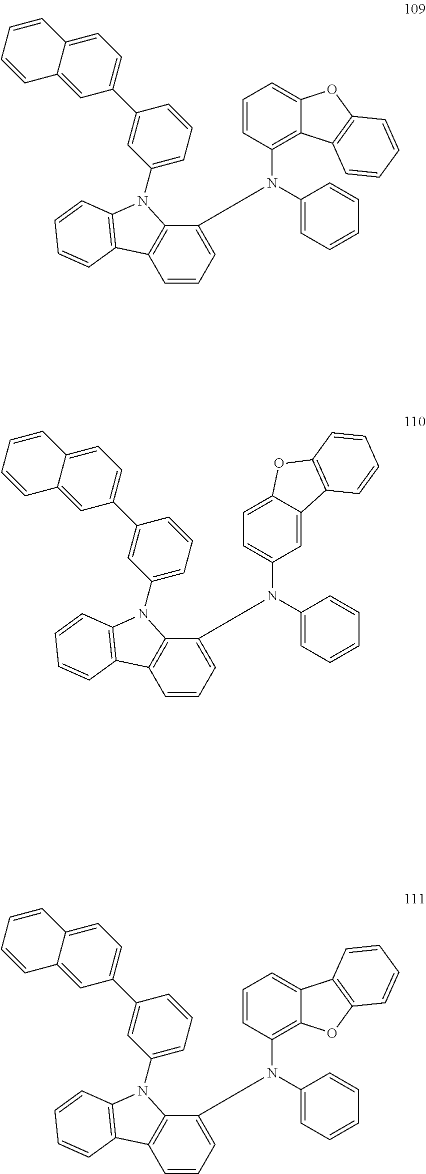 [SMILES: c1ccc(N(c2cccc3oc4ccccc4c23)c2cccc3c4ccccc4n(-c4cccc(-c5ccc6ccccc6c5)c4)c23)cc1 ; c1ccc(N(c2ccc3oc4ccccc4c3c2)c2cccc3c4ccccc4n(-c4cccc(-c5ccc6ccccc6c5)c4)c23)cc1 ; c1ccc(N(c2cccc3c2oc2ccccc23)c2cccc3c4ccccc4n(-c4cccc(-c5ccc6ccccc6c5)c4)c23)cc1] 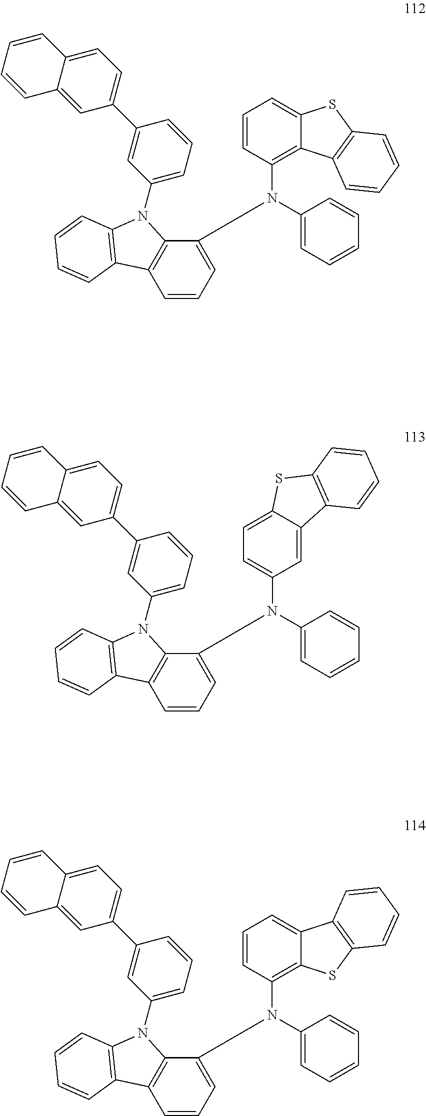 [SMILES: c1ccc(N(c2cccc3sc4ccccc4c23)c2cccc3c4ccccc4n(-c4cccc(-c5ccc6ccccc6c5)c4)c23)cc1 ; c1ccc(N(c2ccc3sc4ccccc4c3c2)c2cccc3c4ccccc4n(-c4cccc(-c5ccc6ccccc6c5)c4)c23)cc1 ; c1ccc(N(c2cccc3c2sc2ccccc23)c2cccc3c4ccccc4n(-c4cccc(-c5ccc6ccccc6c5)c4)c23)cc1] 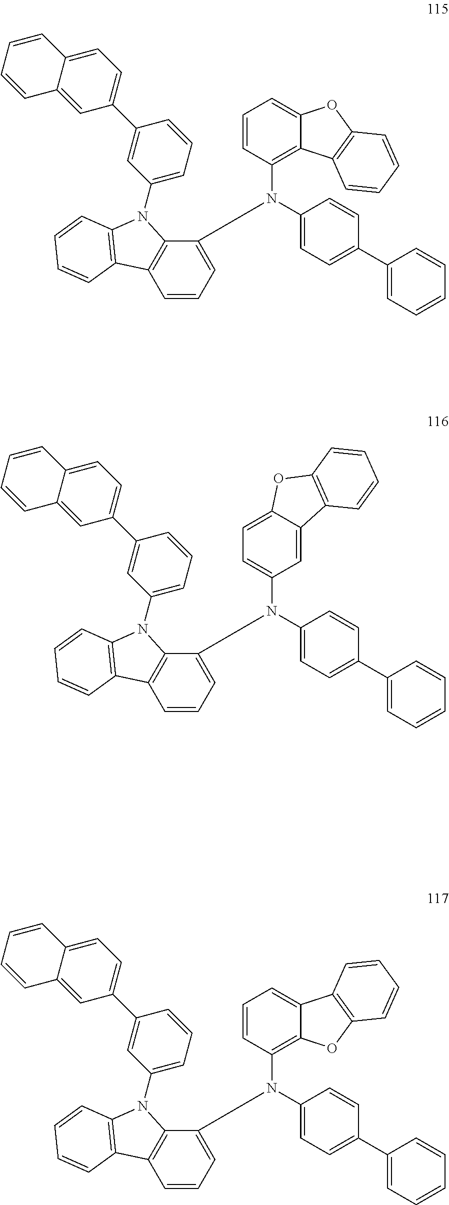 [SMILES: c1ccc(-c2ccc(N(c3cccc4oc5ccccc5c34)c3cccc4c5ccccc5n(-c5cccc(-c6ccc7ccccc7c6)c5)c34)cc2)cc1 ; c1ccc(-c2ccc(N(c3ccc4oc5ccccc5c4c3)c3cccc4c5ccccc5n(-c5cccc(-c6ccc7ccccc7c6)c5)c34)cc2)cc1 ; c1ccc(-c2ccc(N(c3cccc4c3oc3ccccc34)c3cccc4c5ccccc5n(-c5cccc(-c6ccc7ccccc7c6)c5)c34)cc2)cc1] 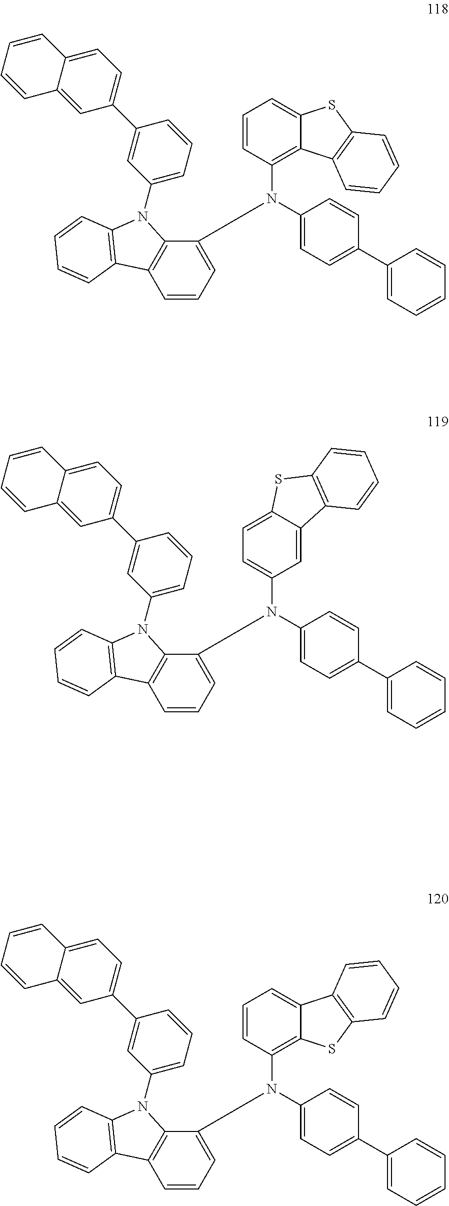 [SMILES: c1ccc(-c2ccc(N(c3cccc4sc5ccccc5c34)c3cccc4c5ccccc5n(-c5cccc(-c6ccc7ccccc7c6)c5)c34)cc2)cc1 ; c1ccc(-c2ccc(N(c3ccc4sc5ccccc5c4c3)c3cccc4c5ccccc5n(-c5cccc(-c6ccc7ccccc7c6)c5)c34)cc2)cc1 ; c1ccc(-c2ccc(N(c3cccc4c3sc3ccccc34)c3cccc4c5ccccc5n(-c5cccc(-c6ccc7ccccc7c6)c5)c34)cc2)cc1] 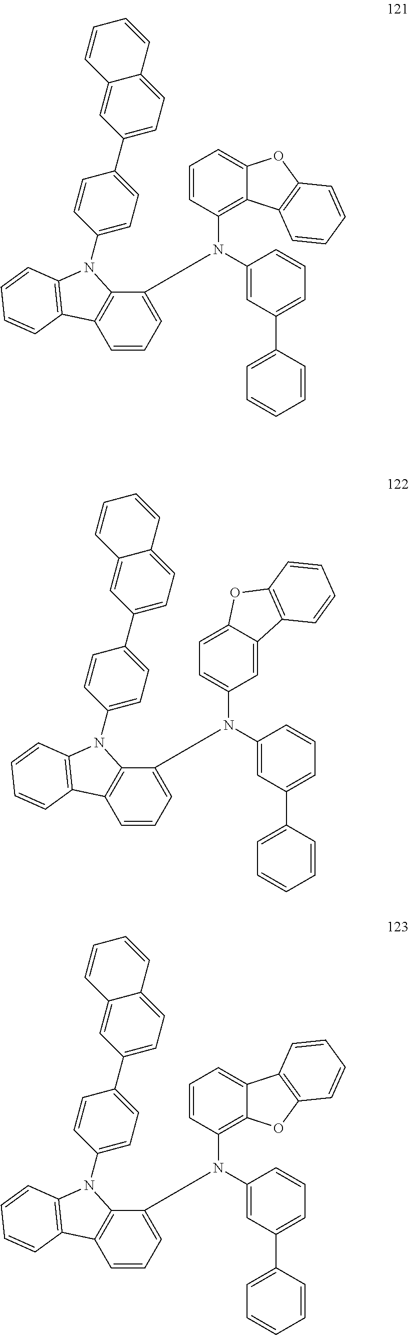 [SMILES: c1ccc(-c2cccc(N(c3cccc4oc5ccccc5c34)c3cccc4c5ccccc5n(-c5ccc(-c6ccc7ccccc7c6)cc5)c34)c2)cc1 ; c1ccc(-c2cccc(N(c3ccc4oc5ccccc5c4c3)c3cccc4c5ccccc5n(-c5ccc(-c6ccc7ccccc7c6)cc5)c34)c2)cc1 ; c1ccc(-c2cccc(N(c3cccc4c3oc3ccccc34)c3cccc4c5ccccc5n(-c5ccc(-c6ccc7ccccc7c6)cc5)c34)c2)cc1] 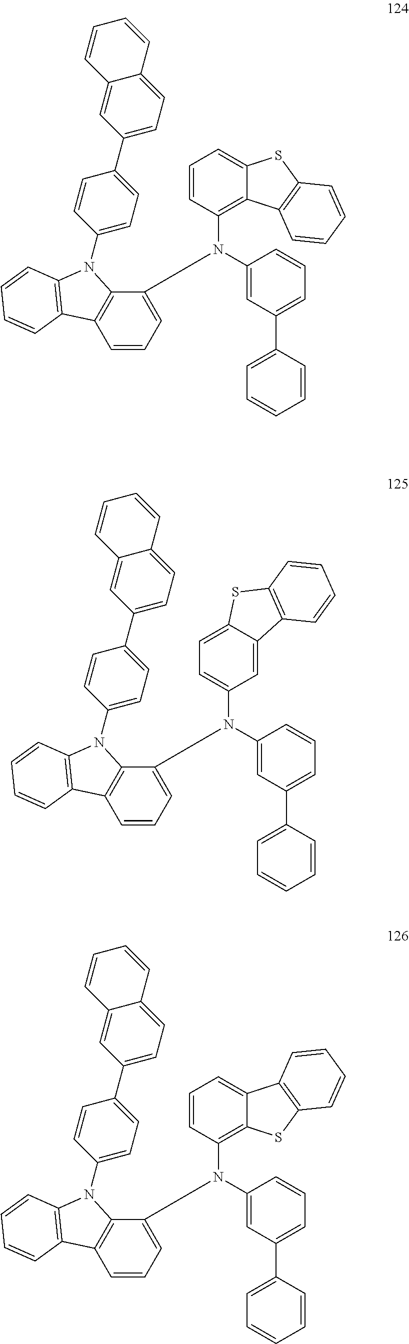 [SMILES: c1ccc(-c2cccc(N(c3cccc4sc5ccccc5c34)c3cccc4c5ccccc5n(-c5ccc(-c6ccc7ccccc7c6)cc5)c34)c2)cc1 ; c1ccc(-c2cccc(N(c3ccc4sc5ccccc5c4c3)c3cccc4c5ccccc5n(-c5ccc(-c6ccc7ccccc7c6)cc5)c34)c2)cc1 ; c1ccc(-c2cccc(N(c3cccc4c3sc3ccccc34)c3cccc4c5ccccc5n(-c5ccc(-c6ccc7ccccc7c6)cc5)c34)c2)cc1] 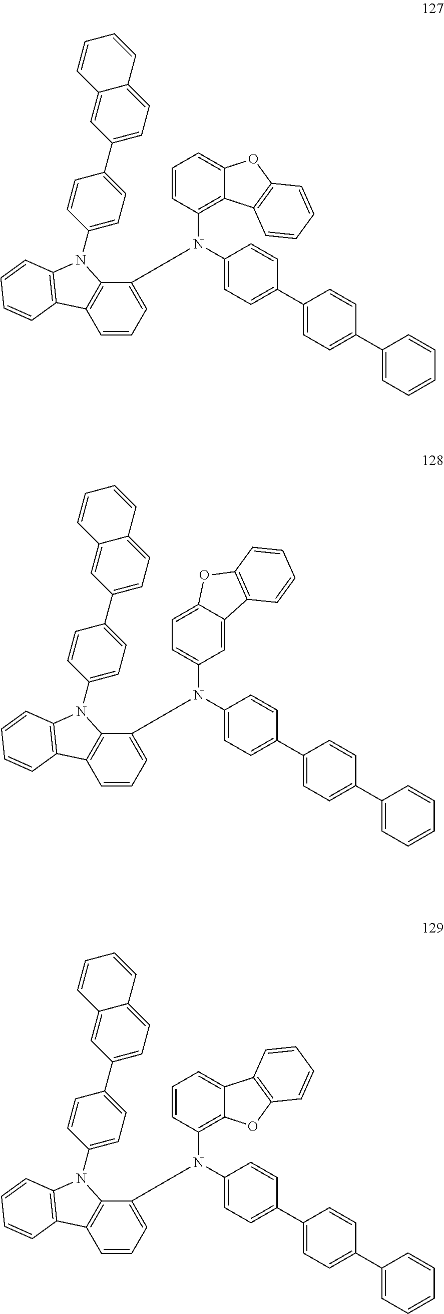 [SMILES: c1ccc(-c2ccc(-c3ccc(N(c4cccc5oc6ccccc6c45)c4cccc5c6ccccc6n(-c6ccc(-c7ccc8ccccc8c7)cc6)c45)cc3)cc2)cc1 ; c1ccc(-c2ccc(-c3ccc(N(c4ccc5oc6ccccc6c5c4)c4cccc5c6ccccc6n(-c6ccc(-c7ccc8ccccc8c7)cc6)c45)cc3)cc2)cc1 ; c1ccc(-c2ccc(-c3ccc(N(c4cccc5c4oc4ccccc45)c4cccc5c6ccccc6n(-c6ccc(-c7ccc8ccccc8c7)cc6)c45)cc3)cc2)cc1] 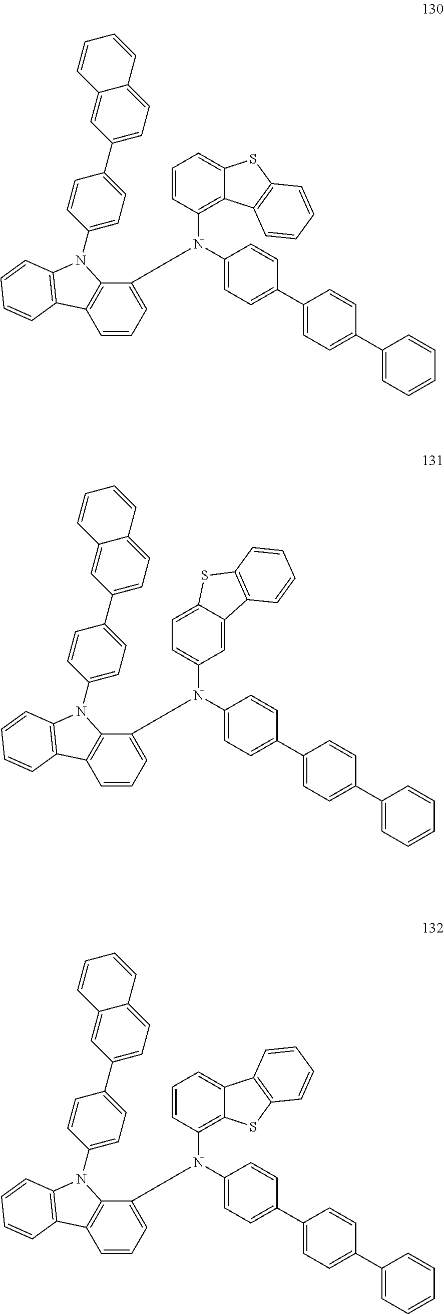 [SMILES: c1ccc(-c2ccc(-c3ccc(N(c4cccc5sc6ccccc6c45)c4cccc5c6ccccc6n(-c6ccc(-c7ccc8ccccc8c7)cc6)c45)cc3)cc2)cc1 ; c1ccc(-c2ccc(-c3ccc(N(c4ccc5sc6ccccc6c5c4)c4cccc5c6ccccc6n(-c6ccc(-c7ccc8ccccc8c7)cc6)c45)cc3)cc2)cc1 ; c1ccc(-c2ccc(-c3ccc(N(c4cccc5c4sc4ccccc45)c4cccc5c6ccccc6n(-c6ccc(-c7ccc8ccccc8c7)cc6)c45)cc3)cc2)cc1] 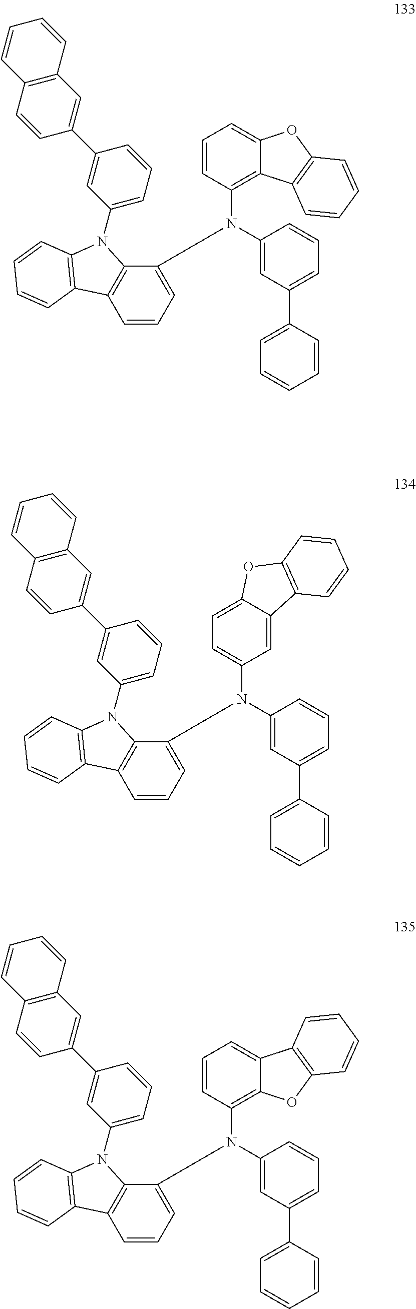 [SMILES: c1ccc(-c2cccc(N(c3cccc4oc5ccccc5c34)c3cccc4c5ccccc5n(-c5cccc(-c6ccc7ccccc7c6)c5)c34)c2)cc1 ; c1ccc(-c2cccc(N(c3ccc4oc5ccccc5c4c3)c3cccc4c5ccccc5n(-c5cccc(-c6ccc7ccccc7c6)c5)c34)c2)cc1 ; c1ccc(-c2cccc(N(c3cccc4c3oc3ccccc34)c3cccc4c5ccccc5n(-c5cccc(-c6ccc7ccccc7c6)c5)c34)c2)cc1] 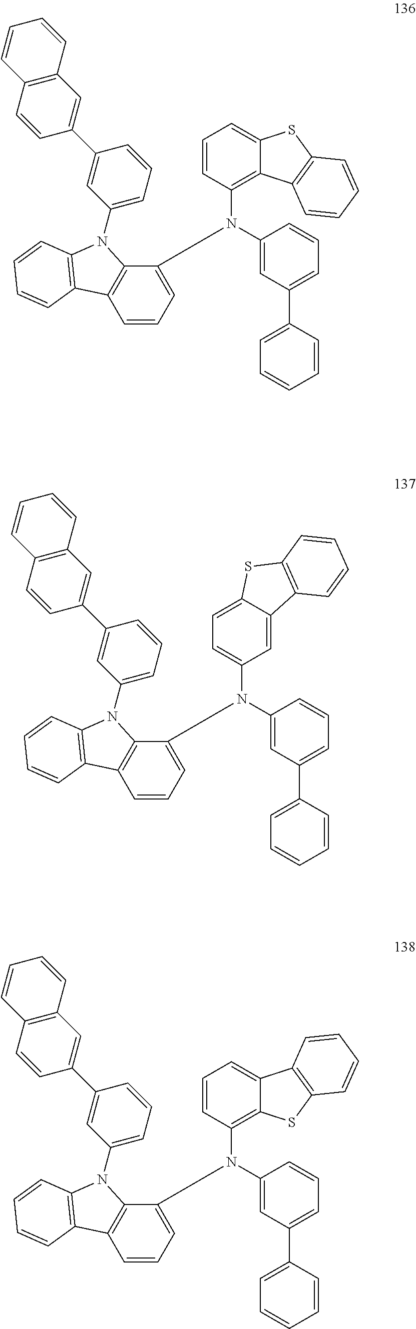 [SMILES: c1ccc(-c2cccc(N(c3cccc4sc5ccccc5c34)c3cccc4c5ccccc5n(-c5cccc(-c6ccc7ccccc7c6)c5)c34)c2)cc1 ; c1ccc(-c2cccc(N(c3ccc4sc5ccccc5c4c3)c3cccc4c5ccccc5n(-c5cccc(-c6ccc7ccccc7c6)c5)c34)c2)cc1 ; c1ccc(-c2cccc(N(c3cccc4c3sc3ccccc34)c3cccc4c5ccccc5n(-c5cccc(-c6ccc7ccccc7c6)c5)c34)c2)cc1] 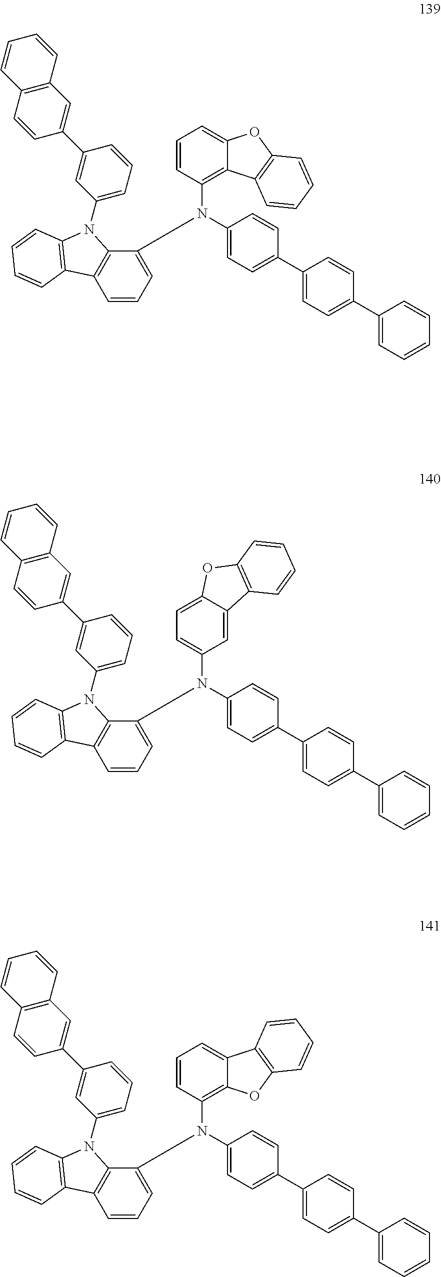 [SMILES: c1ccc(-c2ccc(-c3ccc(N(c4cccc5oc6ccccc6c45)c4cccc5c6ccccc6n(-c6cccc(-c7ccc8ccccc8c7)c6)c45)cc3)cc2)cc1 ; c1ccc(-c2ccc(-c3ccc(N(c4ccc5oc6ccccc6c5c4)c4cccc5c6ccccc6n(-c6cccc(-c7ccc8ccccc8c7)c6)c45)cc3)cc2)cc1 ; c1ccc(-c2ccc(-c3ccc(N(c4cccc5c4oc4ccccc45)c4cccc5c6ccccc6n(-c6cccc(-c7ccc8ccccc8c7)c6)c45)cc3)cc2)cc1] 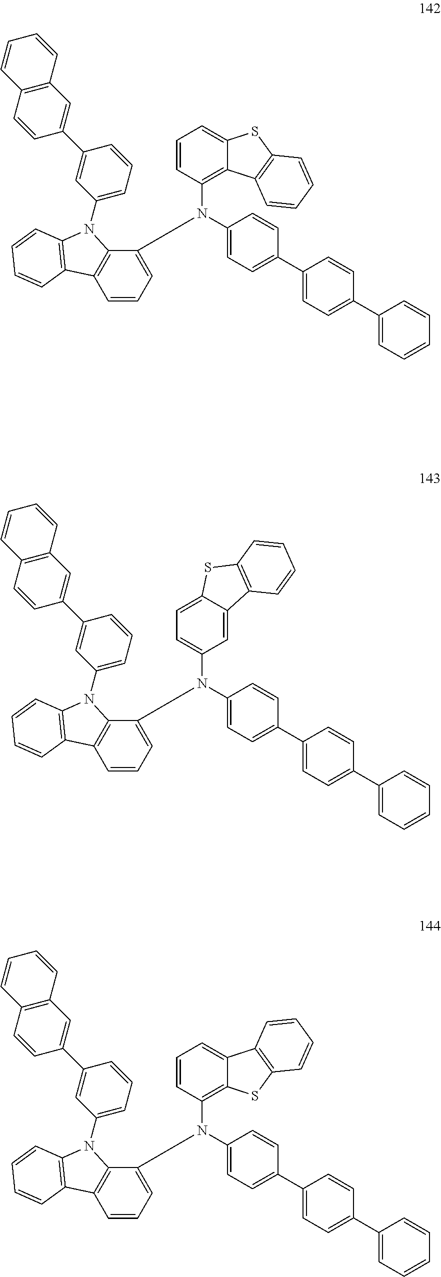 [SMILES: c1ccc(-c2ccc(-c3ccc(N(c4cccc5sc6ccccc6c45)c4cccc5c6ccccc6n(-c6cccc(-c7ccc8ccccc8c7)c6)c45)cc3)cc2)cc1 ; c1ccc(-c2ccc(-c3ccc(N(c4ccc5sc6ccccc6c5c4)c4cccc5c6ccccc6n(-c6cccc(-c7ccc8ccccc8c7)c6)c45)cc3)cc2)cc1 ; c1ccc(-c2ccc(-c3ccc(N(c4cccc5c4sc4ccccc45)c4cccc5c6ccccc6n(-c6cccc(-c7ccc8ccccc8c7)c6)c45)cc3)cc2)cc1] 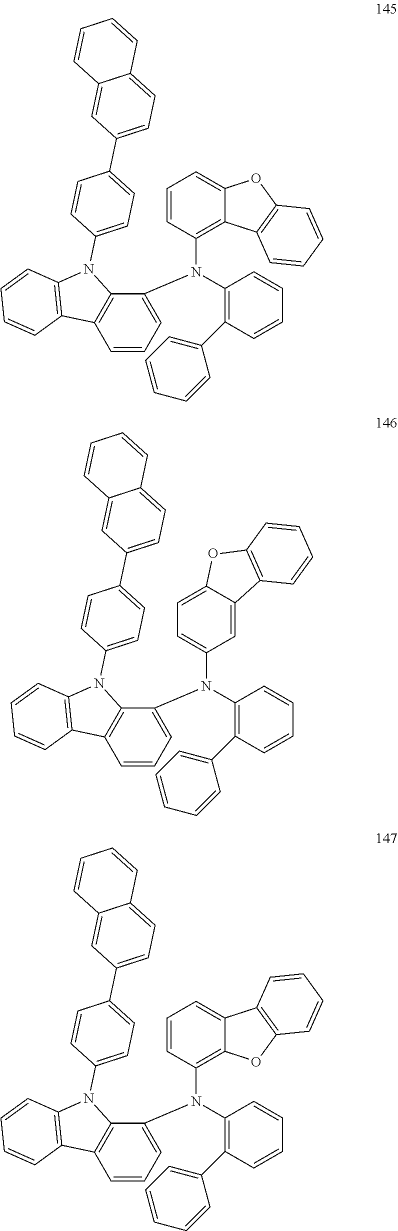 [SMILES: c1ccc(-c2ccccc2N(c2cccc3oc4ccccc4c23)c2cccc3c4ccccc4n(-c4ccc(-c5ccc6ccccc6c5)cc4)c23)cc1 ; c1ccc(-c2ccccc2N(c2ccc3oc4ccccc4c3c2)c2cccc3c4ccccc4n(-c4ccc(-c5ccc6ccccc6c5)cc4)c23)cc1 ; c1ccc(-c2ccccc2N(c2cccc3c2oc2ccccc23)c2cccc3c4ccccc4n(-c4ccc(-c5ccc6ccccc6c5)cc4)c23)cc1] 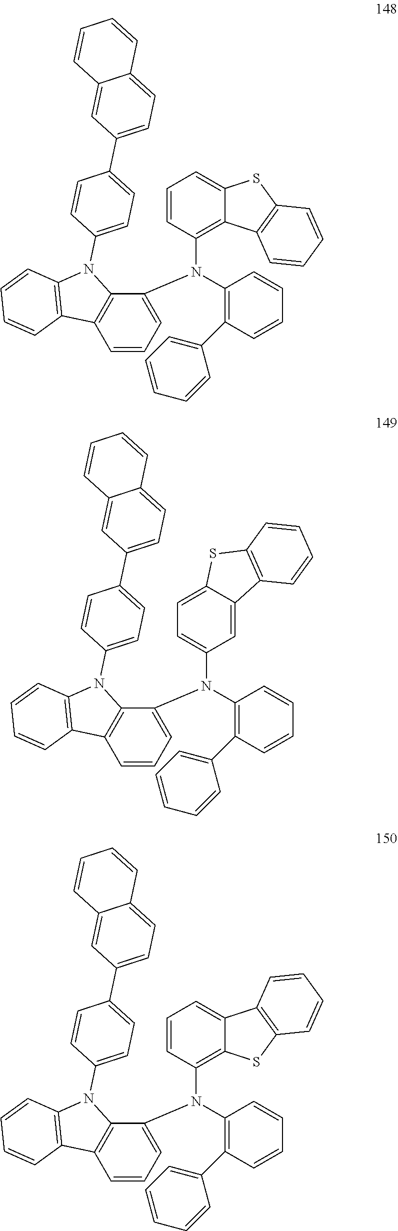 [SMILES: c1ccc(-c2ccccc2N(c2cccc3sc4ccccc4c23)c2cccc3c4ccccc4n(-c4ccc(-c5ccc6ccccc6c5)cc4)c23)cc1 ; c1ccc(-c2ccccc2N(c2ccc3sc4ccccc4c3c2)c2cccc3c4ccccc4n(-c4ccc(-c5ccc6ccccc6c5)cc4)c23)cc1 ; c1ccc(-c2ccccc2N(c2cccc3c2sc2ccccc23)c2cccc3c4ccccc4n(-c4ccc(-c5ccc6ccccc6c5)cc4)c23)cc1] 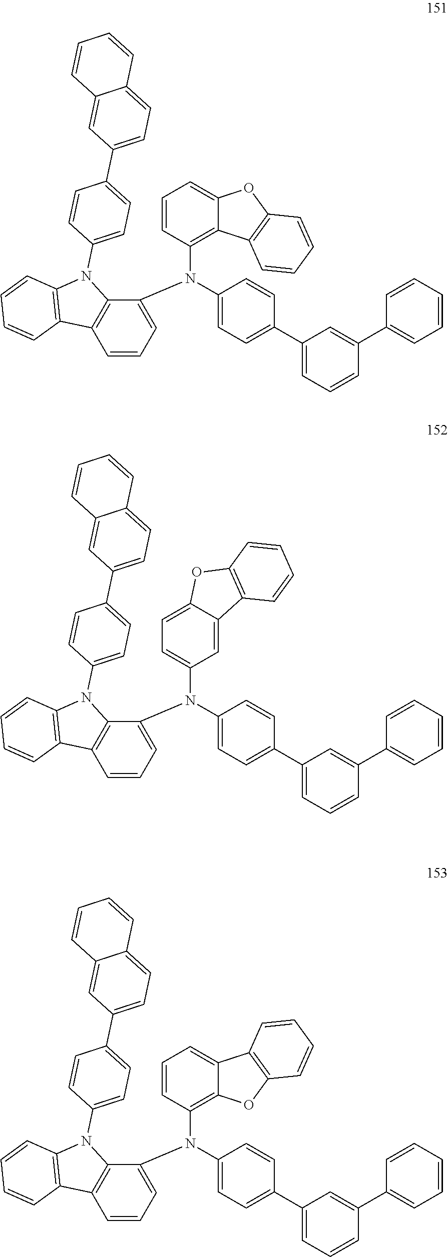 [SMILES: c1ccc(-c2cccc(-c3ccc(N(c4cccc5oc6ccccc6c45)c4cccc5c6ccccc6n(-c6ccc(-c7ccc8ccccc8c7)cc6)c45)cc3)c2)cc1 ; c1ccc(-c2cccc(-c3ccc(N(c4ccc5oc6ccccc6c5c4)c4cccc5c6ccccc6n(-c6ccc(-c7ccc8ccccc8c7)cc6)c45)cc3)c2)cc1 ; c1ccc(-c2cccc(-c3ccc(N(c4cccc5c4oc4ccccc45)c4cccc5c6ccccc6n(-c6ccc(-c7ccc8ccccc8c7)cc6)c45)cc3)c2)cc1] 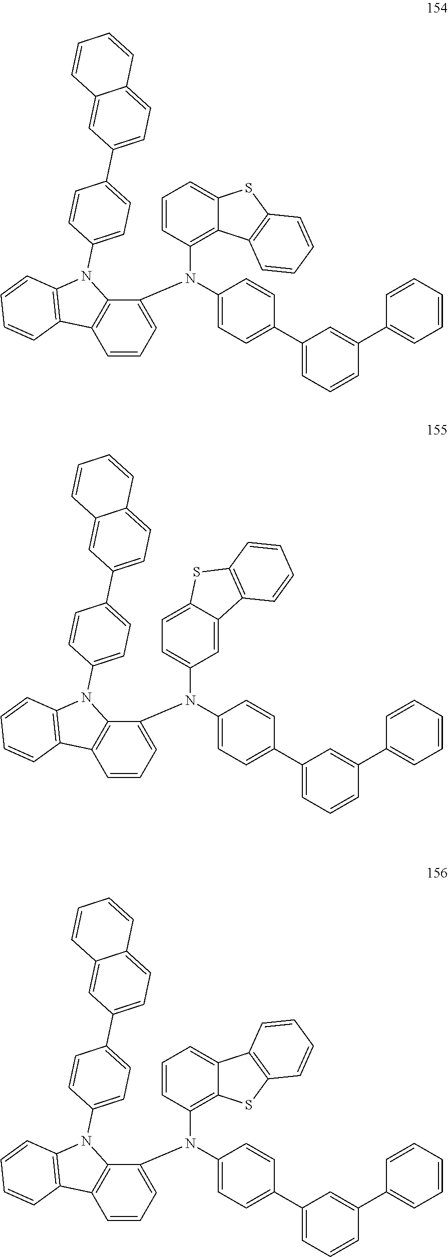 [SMILES: c1ccc(-c2cccc(-c3ccc(N(c4cccc5sc6ccccc6c45)c4cccc5c6ccccc6n(-c6ccc(-c7ccc8ccccc8c7)cc6)c45)cc3)c2)cc1 ; c1ccc(-c2cccc(-c3ccc(N(c4ccc5sc6ccccc6c5c4)c4cccc5c6ccccc6n(-c6ccc(-c7ccc8ccccc8c7)cc6)c45)cc3)c2)cc1 ; c1ccc(-c2cccc(-c3ccc(N(c4cccc5c4sc4ccccc45)c4cccc5c6ccccc6n(-c6ccc(-c7ccc8ccccc8c7)cc6)c45)cc3)c2)cc1] 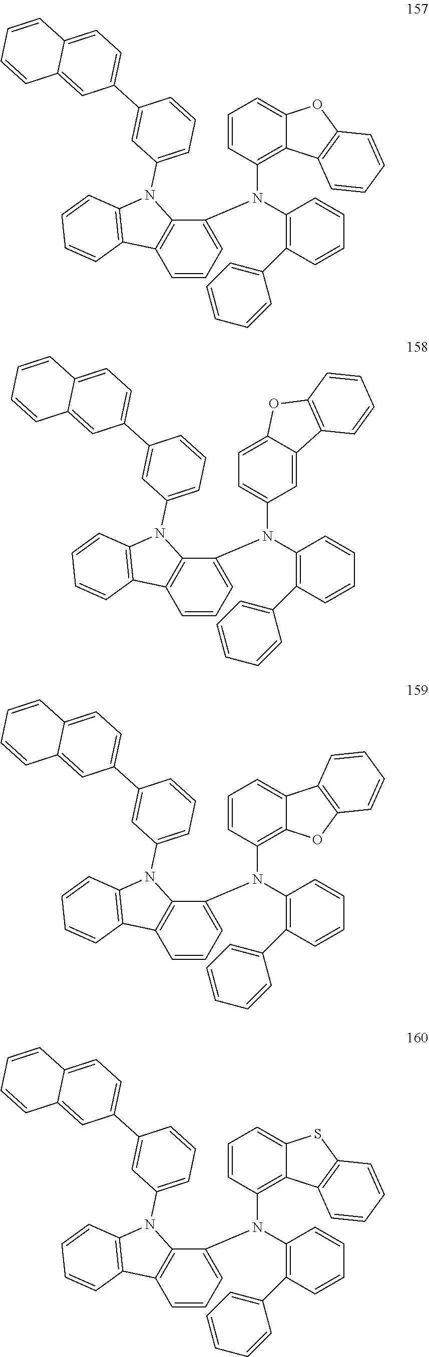 [SMILES: c1ccc(-c2ccccc2N(c2cccc3oc4ccccc4c23)c2cccc3c4ccccc4n(-c4cccc(-c5ccc6ccccc6c5)c4)c23)cc1 ; c1ccc(-c2ccccc2N(c2ccc3oc4ccccc4c3c2)c2cccc3c4ccccc4n(-c4cccc(-c5ccc6ccccc6c5)c4)c23)cc1 ; c1ccc(-c2ccccc2N(c2cccc3c2oc2ccccc23)c2cccc3c4ccccc4n(-c4cccc(-c5ccc6ccccc6c5)c4)c23)cc1 ; c1ccc(-c2ccccc2N(c2cccc3sc4ccccc4c23)c2cccc3c4ccccc4n(-c4cccc(-c5ccc6ccccc6c5)c4)c23)cc1] 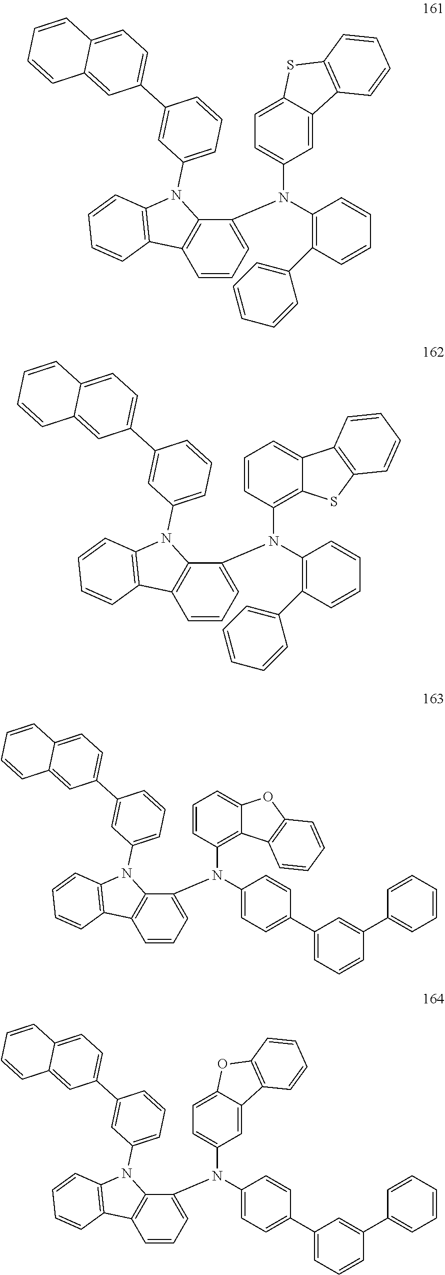 [SMILES: c1ccc(-c2ccccc2N(c2ccc3sc4ccccc4c3c2)c2cccc3c4ccccc4n(-c4cccc(-c5ccc6ccccc6c5)c4)c23)cc1 ; c1ccc(-c2ccccc2N(c2cccc3c2sc2ccccc23)c2cccc3c4ccccc4n(-c4cccc(-c5ccc6ccccc6c5)c4)c23)cc1 ; c1ccc(-c2cccc(-c3ccc(N(c4cccc5oc6ccccc6c45)c4cccc5c6ccccc6n(-c6cccc(-c7ccc8ccccc8c7)c6)c45)cc3)c2)cc1 ; c1ccc(-c2cccc(-c3ccc(N(c4ccc5oc6ccccc6c5c4)c4cccc5c6ccccc6n(-c6cccc(-c7ccc8ccccc8c7)c6)c45)cc3)c2)cc1] 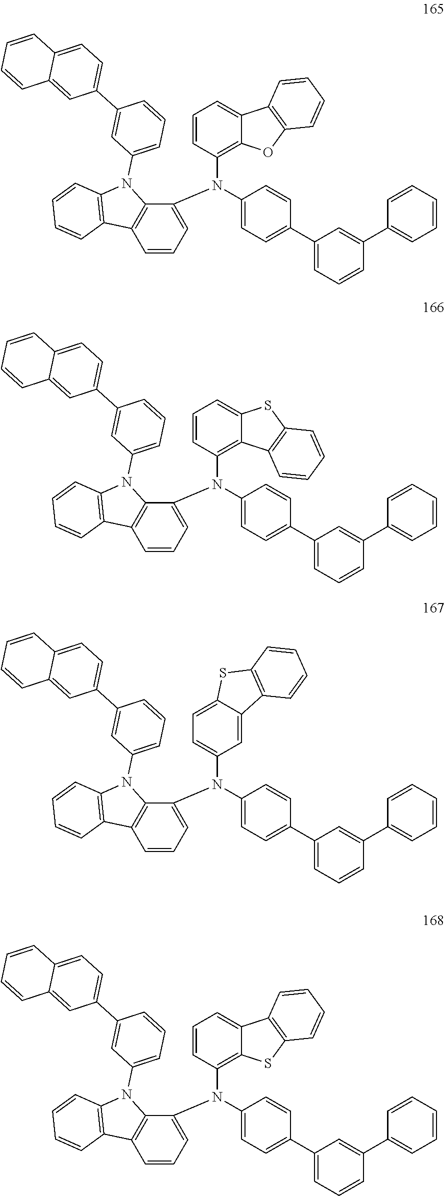 [SMILES: c1ccc(-c2cccc(-c3ccc(N(c4cccc5c4oc4ccccc45)c4cccc5c6ccccc6n(-c6cccc(-c7ccc8ccccc8c7)c6)c45)cc3)c2)cc1 ; c1ccc(-c2cccc(-c3ccc(N(c4cccc5sc6ccccc6c45)c4cccc5c6ccccc6n(-c6cccc(-c7ccc8ccccc8c7)c6)c45)cc3)c2)cc1 ; c1ccc(-c2cccc(-c3ccc(N(c4ccc5sc6ccccc6c5c4)c4cccc5c6ccccc6n(-c6cccc(-c7ccc8ccccc8c7)c6)c45)cc3)c2)cc1 ; c1ccc(-c2cccc(-c3ccc(N(c4cccc5c4sc4ccccc45)c4cccc5c6ccccc6n(-c6cccc(-c7ccc8ccccc8c7)c6)c45)cc3)c2)cc1] 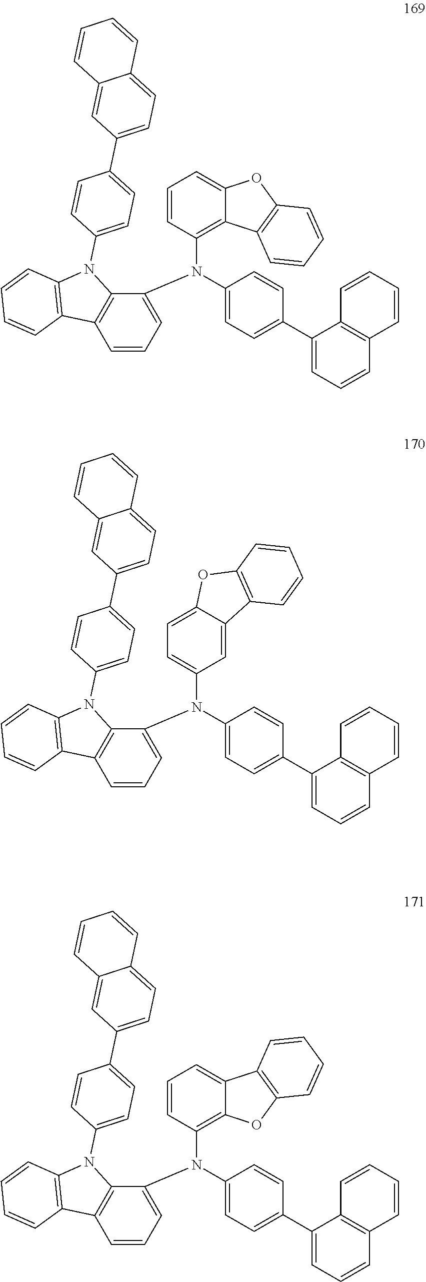 [SMILES: c1ccc2cc(-c3ccc(-n4c5ccccc5c5cccc(N(c6ccc(-c7cccc8ccccc78)cc6)c6cccc7oc8ccccc8c67)c54)cc3)ccc2c1 ; c1ccc2cc(-c3ccc(-n4c5ccccc5c5cccc(N(c6ccc(-c7cccc8ccccc78)cc6)c6ccc7oc8ccccc8c7c6)c54)cc3)ccc2c1 ; c1ccc2cc(-c3ccc(-n4c5ccccc5c5cccc(N(c6ccc(-c7cccc8ccccc78)cc6)c6cccc7c6oc6ccccc67)c54)cc3)ccc2c1] 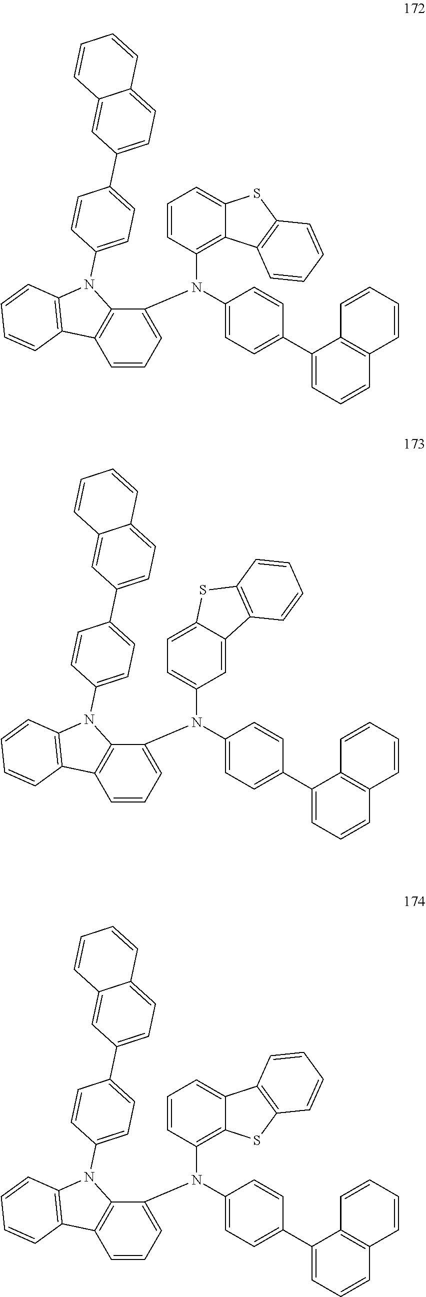 [SMILES: c1ccc2cc(-c3ccc(-n4c5ccccc5c5cccc(N(c6ccc(-c7cccc8ccccc78)cc6)c6cccc7sc8ccccc8c67)c54)cc3)ccc2c1 ; c1ccc2cc(-c3ccc(-n4c5ccccc5c5cccc(N(c6ccc(-c7cccc8ccccc78)cc6)c6ccc7sc8ccccc8c7c6)c54)cc3)ccc2c1 ; c1ccc2cc(-c3ccc(-n4c5ccccc5c5cccc(N(c6ccc(-c7cccc8ccccc78)cc6)c6cccc7c6sc6ccccc67)c54)cc3)ccc2c1] 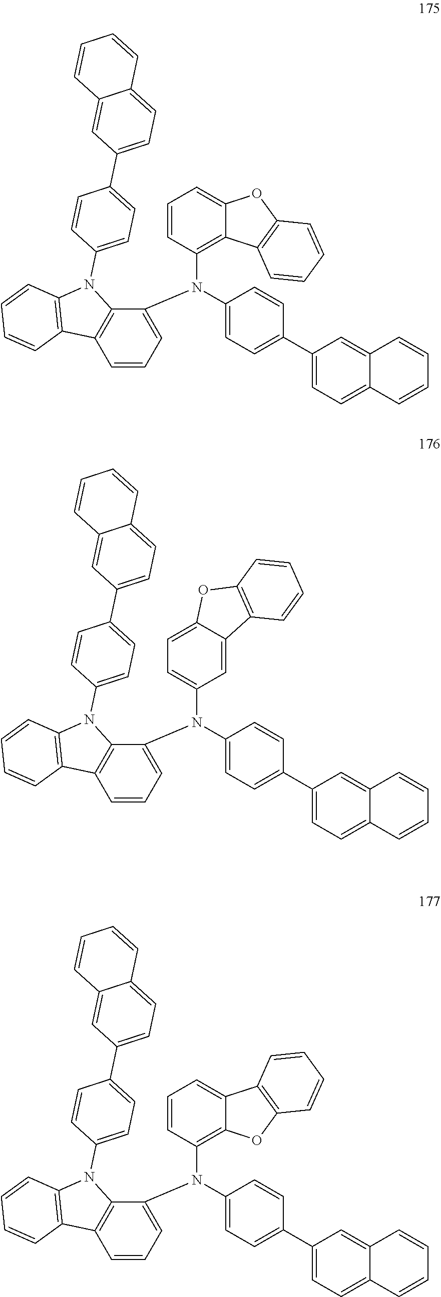 [SMILES: c1ccc2cc(-c3ccc(N(c4cccc5oc6ccccc6c45)c4cccc5c6ccccc6n(-c6ccc(-c7ccc8ccccc8c7)cc6)c45)cc3)ccc2c1 ; c1ccc2cc(-c3ccc(N(c4ccc5oc6ccccc6c5c4)c4cccc5c6ccccc6n(-c6ccc(-c7ccc8ccccc8c7)cc6)c45)cc3)ccc2c1 ; c1ccc2cc(-c3ccc(N(c4cccc5c4oc4ccccc45)c4cccc5c6ccccc6n(-c6ccc(-c7ccc8ccccc8c7)cc6)c45)cc3)ccc2c1] 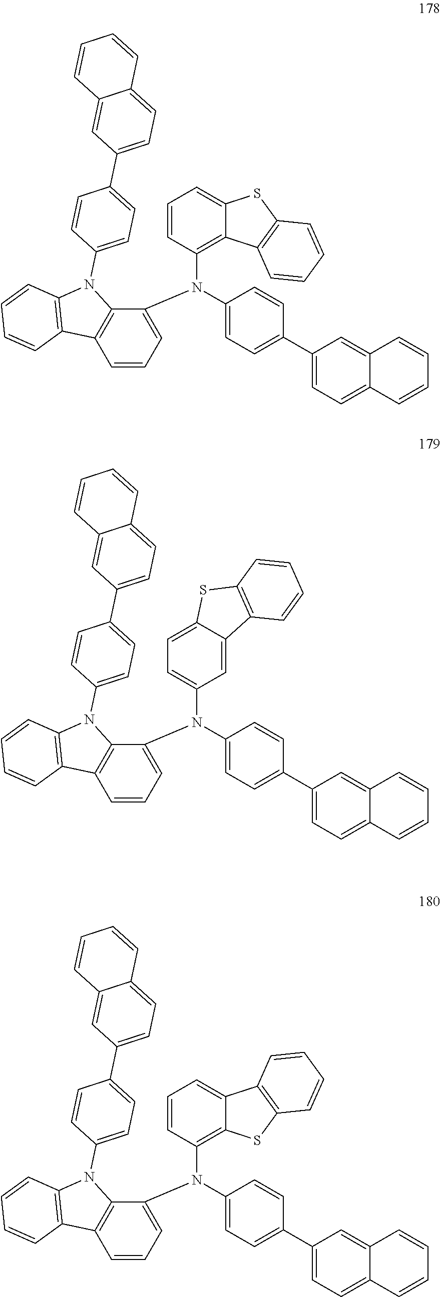 [SMILES: c1ccc2cc(-c3ccc(N(c4cccc5sc6ccccc6c45)c4cccc5c6ccccc6n(-c6ccc(-c7ccc8ccccc8c7)cc6)c45)cc3)ccc2c1 ; c1ccc2cc(-c3ccc(N(c4ccc5sc6ccccc6c5c4)c4cccc5c6ccccc6n(-c6ccc(-c7ccc8ccccc8c7)cc6)c45)cc3)ccc2c1 ; c1ccc2cc(-c3ccc(N(c4cccc5c4sc4ccccc45)c4cccc5c6ccccc6n(-c6ccc(-c7ccc8ccccc8c7)cc6)c45)cc3)ccc2c1] 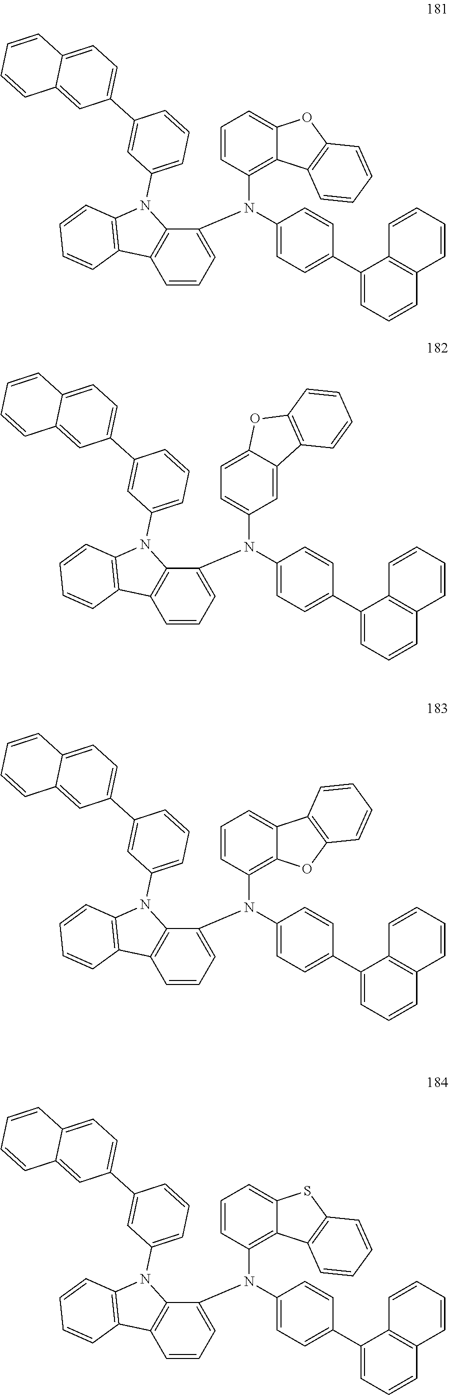 [SMILES: c1cc(-c2ccc3ccccc3c2)cc(-n2c3ccccc3c3cccc(N(c4ccc(-c5cccc6ccccc56)cc4)c4cccc5oc6ccccc6c45)c32)c1 ; c1cc(-c2ccc3ccccc3c2)cc(-n2c3ccccc3c3cccc(N(c4ccc(-c5cccc6ccccc56)cc4)c4ccc5oc6ccccc6c5c4)c32)c1 ; c1cc(-c2ccc3ccccc3c2)cc(-n2c3ccccc3c3cccc(N(c4ccc(-c5cccc6ccccc56)cc4)c4cccc5c4oc4ccccc45)c32)c1 ; c1cc(-c2ccc3ccccc3c2)cc(-n2c3ccccc3c3cccc(N(c4ccc(-c5cccc6ccccc56)cc4)c4cccc5sc6ccccc6c45)c32)c1] 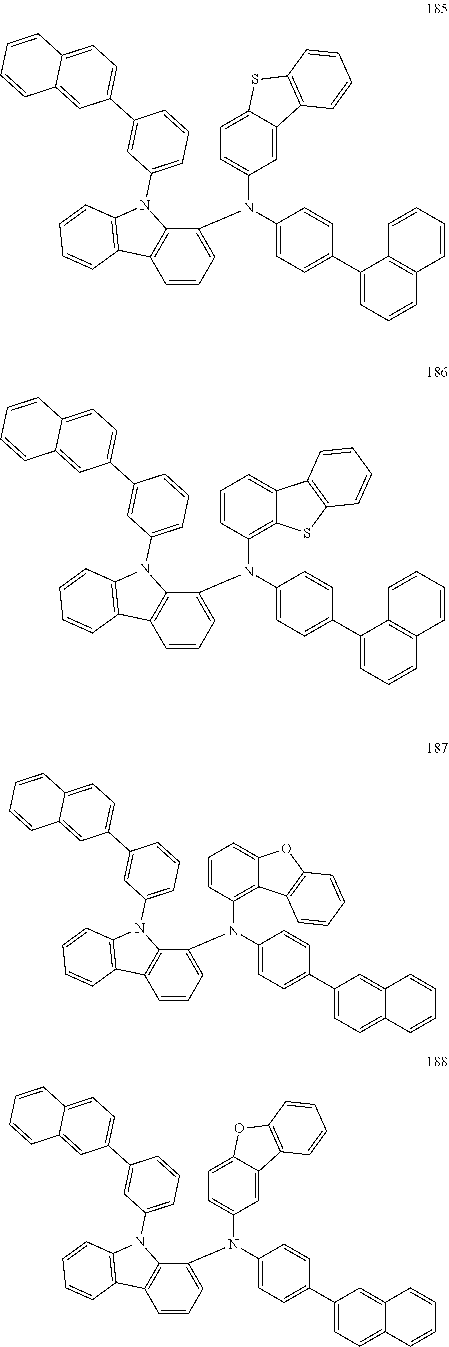 [SMILES: c1cc(-c2ccc3ccccc3c2)cc(-n2c3ccccc3c3cccc(N(c4ccc(-c5cccc6ccccc56)cc4)c4ccc5sc6ccccc6c5c4)c32)c1 ; c1cc(-c2ccc3ccccc3c2)cc(-n2c3ccccc3c3cccc(N(c4ccc(-c5cccc6ccccc56)cc4)c4cccc5c4sc4ccccc45)c32)c1 ; c1cc(-c2ccc3ccccc3c2)cc(-n2c3ccccc3c3cccc(N(c4ccc(-c5ccc6ccccc6c5)cc4)c4cccc5oc6ccccc6c45)c32)c1 ; c1cc(-c2ccc3ccccc3c2)cc(-n2c3ccccc3c3cccc(N(c4ccc(-c5ccc6ccccc6c5)cc4)c4ccc5oc6ccccc6c5c4)c32)c1] 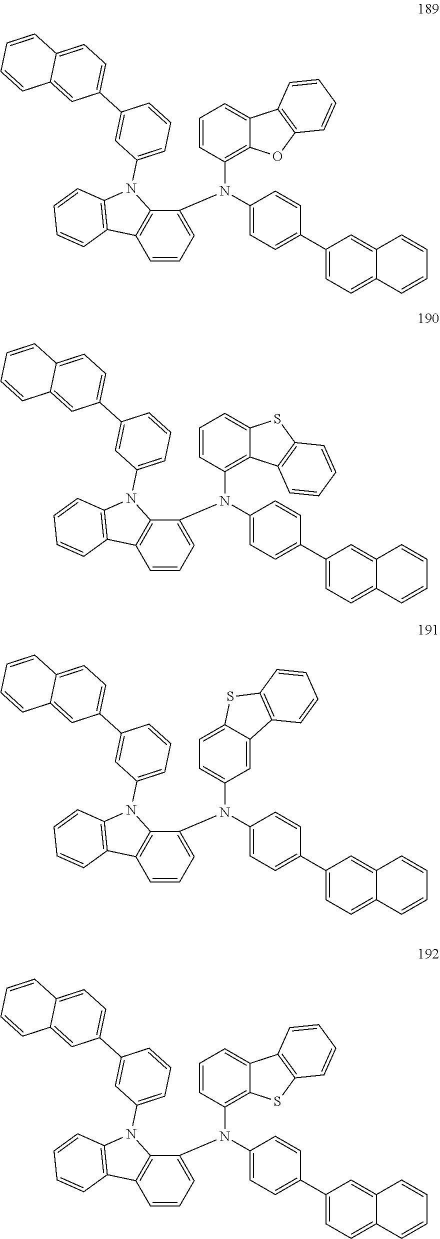 [SMILES: c1cc(-c2ccc3ccccc3c2)cc(-n2c3ccccc3c3cccc(N(c4ccc(-c5ccc6ccccc6c5)cc4)c4cccc5c4oc4ccccc45)c32)c1 ; c1cc(-c2ccc3ccccc3c2)cc(-n2c3ccccc3c3cccc(N(c4ccc(-c5ccc6ccccc6c5)cc4)c4cccc5sc6ccccc6c45)c32)c1 ; c1cc(-c2ccc3ccccc3c2)cc(-n2c3ccccc3c3cccc(N(c4ccc(-c5ccc6ccccc6c5)cc4)c4ccc5sc6ccccc6c5c4)c32)c1 ; c1cc(-c2ccc3ccccc3c2)cc(-n2c3ccccc3c3cccc(N(c4ccc(-c5ccc6ccccc6c5)cc4)c4cccc5c4sc4ccccc45)c32)c1] 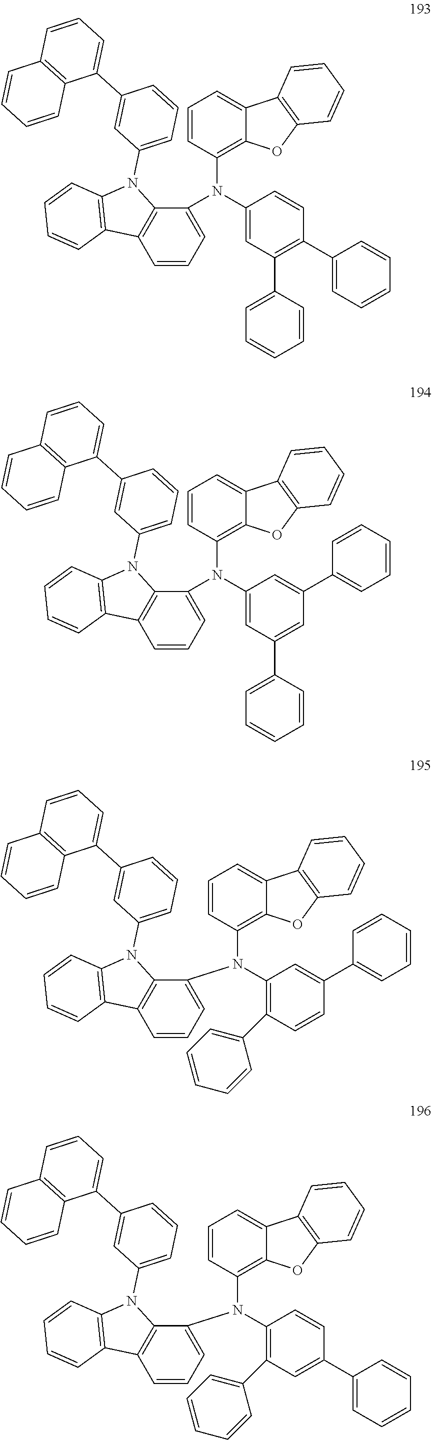 [SMILES: c1ccc(-c2ccc(N(c3cccc4c3oc3ccccc34)c3cccc4c5ccccc5n(-c5cccc(-c6cccc7ccccc67)c5)c34)cc2-c2ccccc2)cc1 ; c1ccc(-c2cc(-c3ccccc3)cc(N(c3cccc4c3oc3ccccc34)c3cccc4c5ccccc5n(-c5cccc(-c6cccc7ccccc67)c5)c34)c2)cc1 ; c1ccc(-c2ccc(-c3ccccc3)c(N(c3cccc4c3oc3ccccc34)c3cccc4c5ccccc5n(-c5cccc(-c6cccc7ccccc67)c5)c34)c2)cc1 ; c1ccc(-c2ccc(N(c3cccc4c3oc3ccccc34)c3cccc4c5ccccc5n(-c5cccc(-c6cccc7ccccc67)c5)c34)c(-c3ccccc3)c2)cc1] 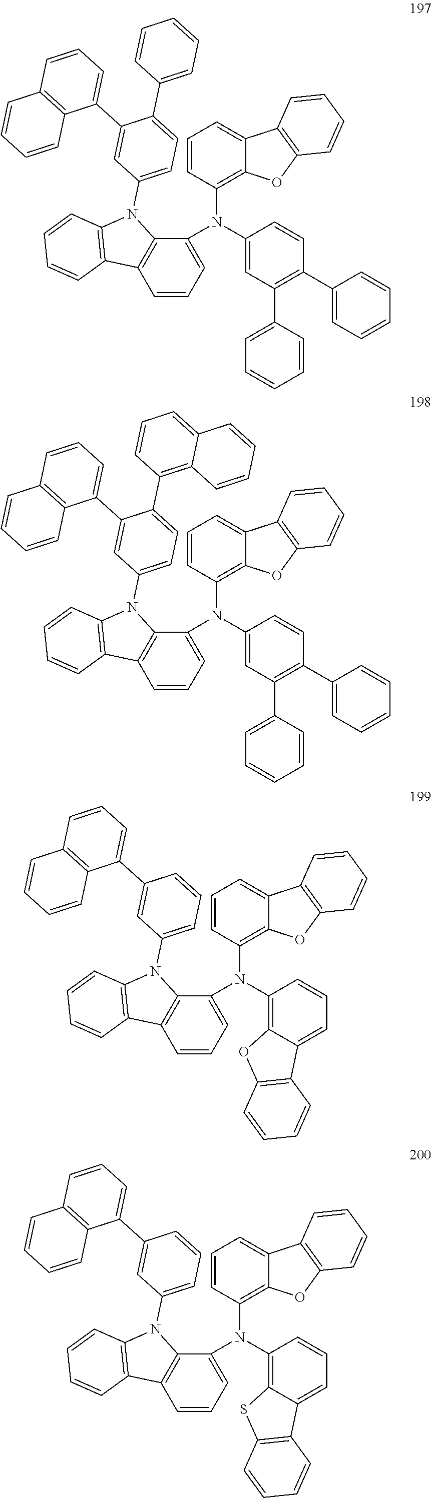 [SMILES: c1ccc(-c2ccc(N(c3cccc4c3oc3ccccc34)c3cccc4c5ccccc5n(-c5ccc(-c6ccccc6)c(-c6cccc7ccccc67)c5)c34)cc2-c2ccccc2)cc1 ; c1ccc(-c2ccc(N(c3cccc4c3oc3ccccc34)c3cccc4c5ccccc5n(-c5ccc(-c6cccc7ccccc67)c(-c6cccc7ccccc67)c5)c34)cc2-c2ccccc2)cc1 ; c1cc(-c2cccc3ccccc23)cc(-n2c3ccccc3c3cccc(N(c4cccc5c4oc4ccccc45)c4cccc5c4oc4ccccc45)c32)c1 ; c1cc(-c2cccc3ccccc23)cc(-n2c3ccccc3c3cccc(N(c4cccc5c4oc4ccccc45)c4cccc5c4sc4ccccc45)c32)c1] 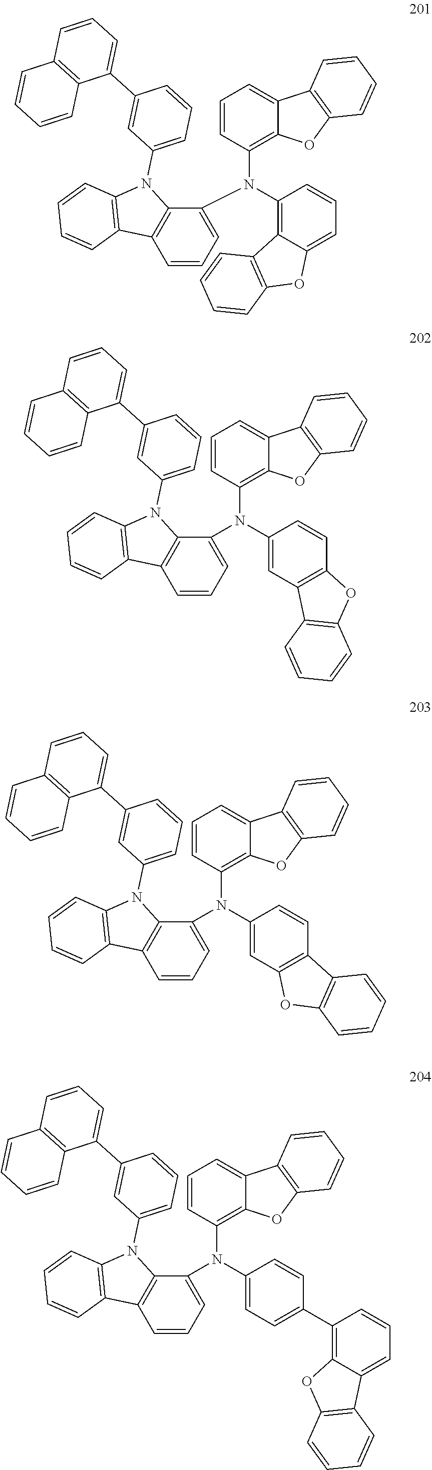 [SMILES: c1cc(-c2cccc3ccccc23)cc(-n2c3ccccc3c3cccc(N(c4cccc5c4oc4ccccc45)c4cccc5oc6ccccc6c45)c32)c1 ; c1cc(-c2cccc3ccccc23)cc(-n2c3ccccc3c3cccc(N(c4ccc5oc6ccccc6c5c4)c4cccc5c4oc4ccccc45)c32)c1 ; c1cc(-c2cccc3ccccc23)cc(-n2c3ccccc3c3cccc(N(c4ccc5c(c4)oc4ccccc45)c4cccc5c4oc4ccccc45)c32)c1 ; c1cc(-c2cccc3ccccc23)cc(-n2c3ccccc3c3cccc(N(c4ccc(-c5cccc6c5oc5ccccc56)cc4)c4cccc5c4oc4ccccc45)c32)c1] 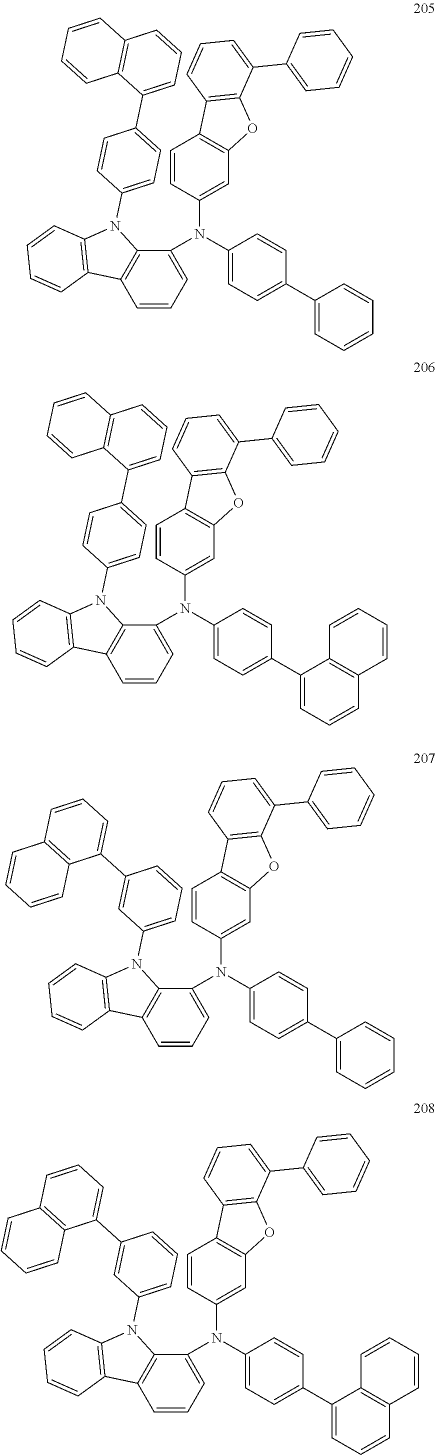 [SMILES: c1ccc(-c2ccc(N(c3ccc4c(c3)oc3c(-c5ccccc5)cccc34)c3cccc4c5ccccc5n(-c5ccc(-c6cccc7ccccc67)cc5)c34)cc2)cc1 ; c1ccc(-c2cccc3c2oc2cc(N(c4ccc(-c5cccc6ccccc56)cc4)c4cccc5c6ccccc6n(-c6ccc(-c7cccc8ccccc78)cc6)c45)ccc23)cc1 ; c1ccc(-c2ccc(N(c3ccc4c(c3)oc3c(-c5ccccc5)cccc34)c3cccc4c5ccccc5n(-c5cccc(-c6cccc7ccccc67)c5)c34)cc2)cc1 ; c1ccc(-c2cccc3c2oc2cc(N(c4ccc(-c5cccc6ccccc56)cc4)c4cccc5c6ccccc6n(-c6cccc(-c7cccc8ccccc78)c6)c45)ccc23)cc1] 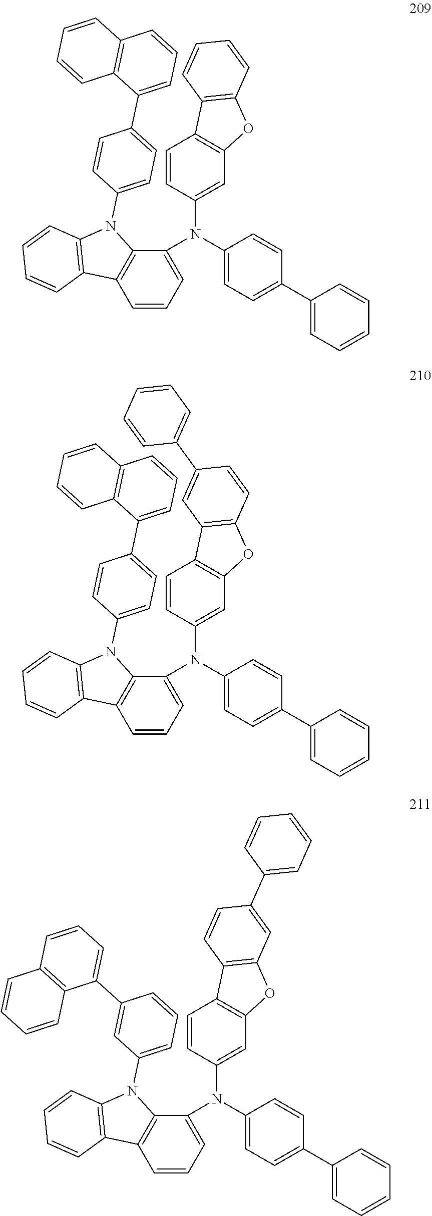 [SMILES: c1ccc(-c2ccc(N(c3ccc4c(c3)oc3ccccc34)c3cccc4c5ccccc5n(-c5ccc(-c6cccc7ccccc67)cc5)c34)cc2)cc1 ; c1ccc(-c2ccc(N(c3ccc4c(c3)oc3ccc(-c5ccccc5)cc34)c3cccc4c5ccccc5n(-c5ccc(-c6cccc7ccccc67)cc5)c34)cc2)cc1 ; c1ccc(-c2ccc(N(c3ccc4c(c3)oc3cc(-c5ccccc5)ccc34)c3cccc4c5ccccc5n(-c5cccc(-c6cccc7ccccc67)c5)c34)cc2)cc1] 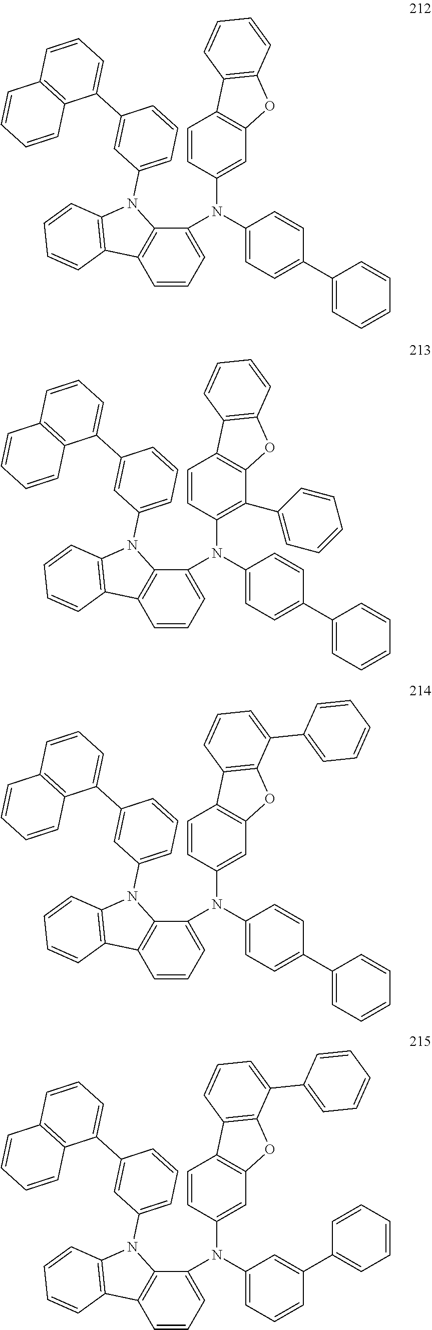 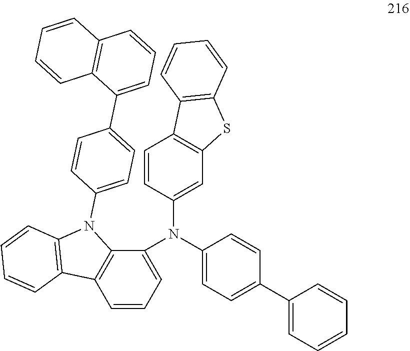 [SMILES: c1ccc(-c2ccc(N(c3ccc4c(c3)sc3ccccc34)c3cccc4c5ccccc5n(-c5ccc(-c6cccc7ccccc67)cc5)c34)cc2)cc1] 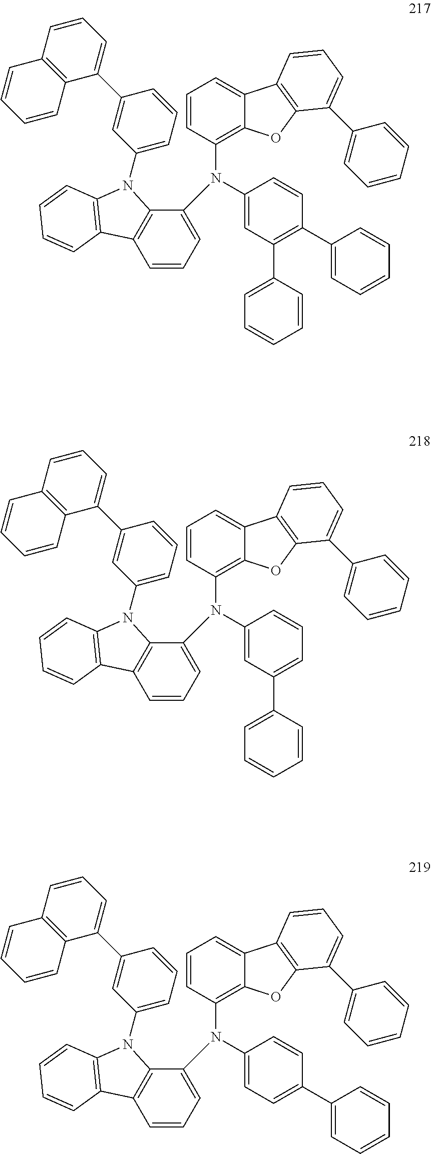 [SMILES: c1ccc(-c2ccc(N(c3cccc4c3oc3c(-c5ccccc5)cccc34)c3cccc4c5ccccc5n(-c5cccc(-c6cccc7ccccc67)c5)c34)cc2-c2ccccc2)cc1 ; c1ccc(-c2cccc(N(c3cccc4c3oc3c(-c5ccccc5)cccc34)c3cccc4c5ccccc5n(-c5cccc(-c6cccc7ccccc67)c5)c34)c2)cc1 ; c1ccc(-c2ccc(N(c3cccc4c3oc3c(-c5ccccc5)cccc34)c3cccc4c5ccccc5n(-c5cccc(-c6cccc7ccccc67)c5)c34)cc2)cc1] 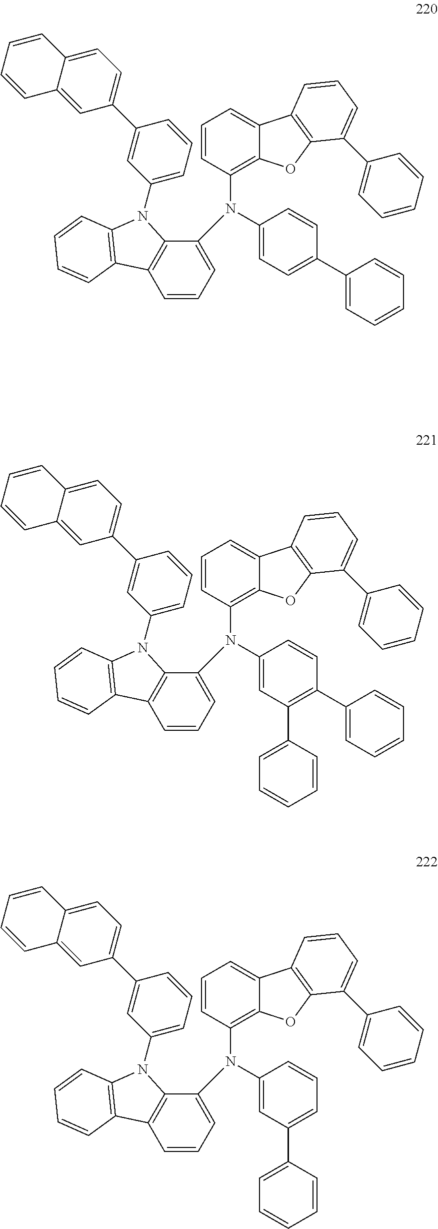 [SMILES: c1ccc(-c2ccc(N(c3cccc4c3oc3c(-c5ccccc5)cccc34)c3cccc4c5ccccc5n(-c5cccc(-c6ccc7ccccc7c6)c5)c34)cc2)cc1 ; c1ccc(-c2ccc(N(c3cccc4c3oc3c(-c5ccccc5)cccc34)c3cccc4c5ccccc5n(-c5cccc(-c6ccc7ccccc7c6)c5)c34)cc2-c2ccccc2)cc1 ; c1ccc(-c2cccc(N(c3cccc4c3oc3c(-c5ccccc5)cccc34)c3cccc4c5ccccc5n(-c5cccc(-c6ccc7ccccc7c6)c5)c34)c2)cc1] 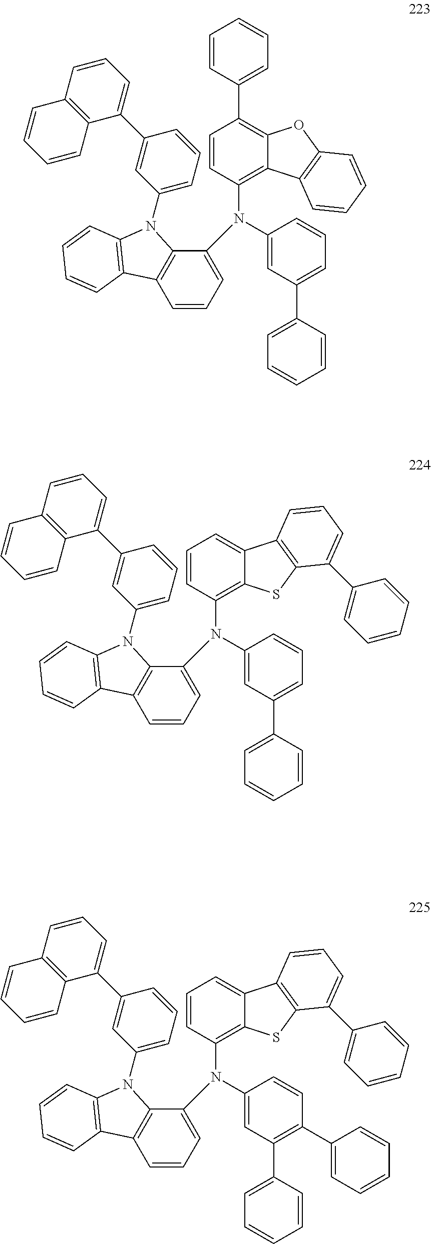 [SMILES: c1ccc(-c2cccc(N(c3ccc(-c4ccccc4)c4oc5ccccc5c34)c3cccc4c5ccccc5n(-c5cccc(-c6cccc7ccccc67)c5)c34)c2)cc1 ; c1ccc(-c2cccc(N(c3cccc4c3sc3c(-c5ccccc5)cccc34)c3cccc4c5ccccc5n(-c5cccc(-c6cccc7ccccc67)c5)c34)c2)cc1 ; c1ccc(-c2ccc(N(c3cccc4c3sc3c(-c5ccccc5)cccc34)c3cccc4c5ccccc5n(-c5cccc(-c6cccc7ccccc67)c5)c34)cc2-c2ccccc2)cc1] 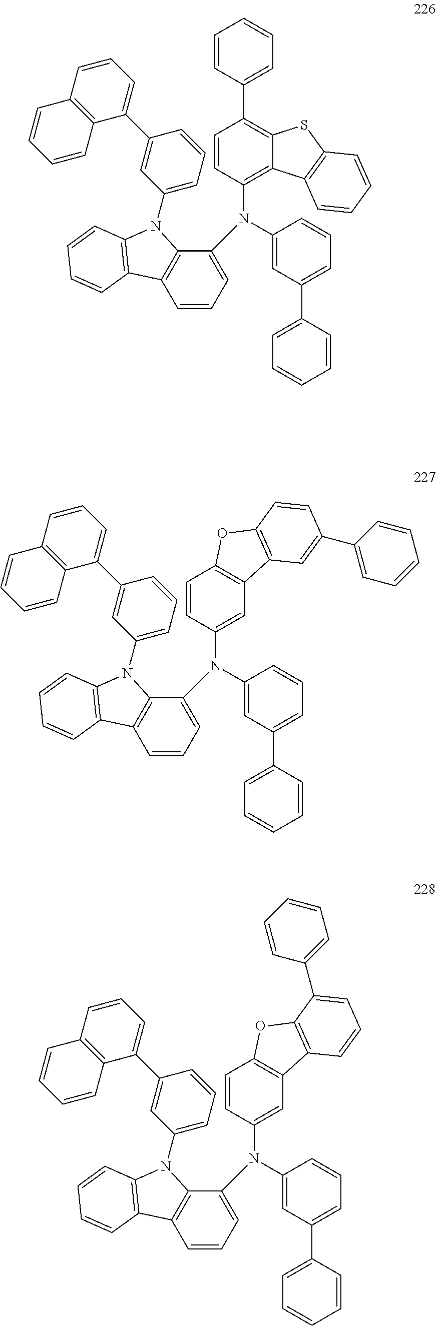 [SMILES: c1ccc(-c2cccc(N(c3ccc(-c4ccccc4)c4sc5ccccc5c34)c3cccc4c5ccccc5n(-c5cccc(-c6cccc7ccccc67)c5)c34)c2)cc1 ; c1ccc(-c2cccc(N(c3ccc4oc5ccc(-c6ccccc6)cc5c4c3)c3cccc4c5ccccc5n(-c5cccc(-c6cccc7ccccc67)c5)c34)c2)cc1 ; c1ccc(-c2cccc(N(c3ccc4oc5c(-c6ccccc6)cccc5c4c3)c3cccc4c5ccccc5n(-c5cccc(-c6cccc7ccccc67)c5)c34)c2)cc1] 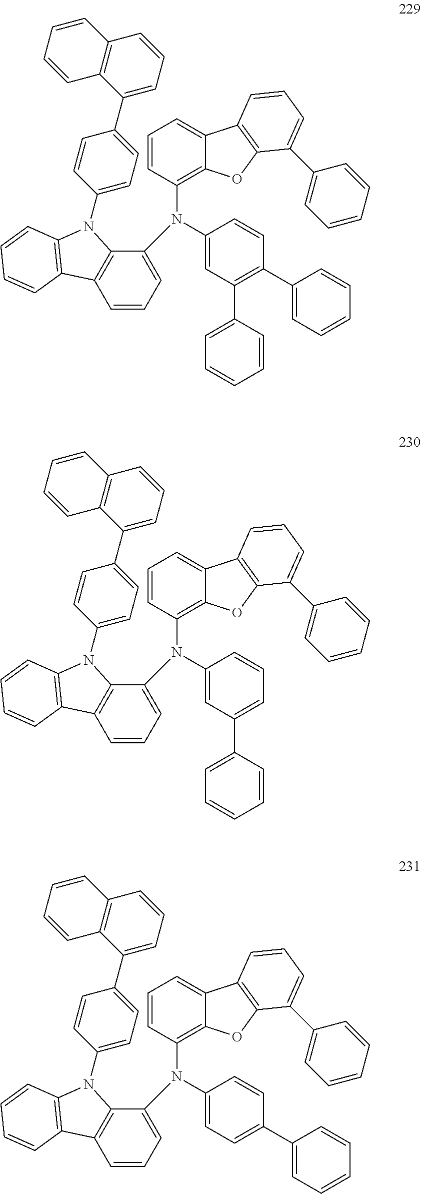 [SMILES: c1ccc(-c2ccc(N(c3cccc4c3oc3c(-c5ccccc5)cccc34)c3cccc4c5ccccc5n(-c5ccc(-c6cccc7ccccc67)cc5)c34)cc2-c2ccccc2)cc1 ; c1ccc(-c2cccc(N(c3cccc4c3oc3c(-c5ccccc5)cccc34)c3cccc4c5ccccc5n(-c5ccc(-c6cccc7ccccc67)cc5)c34)c2)cc1 ; c1ccc(-c2ccc(N(c3cccc4c3oc3c(-c5ccccc5)cccc34)c3cccc4c5ccccc5n(-c5ccc(-c6cccc7ccccc67)cc5)c34)cc2)cc1] 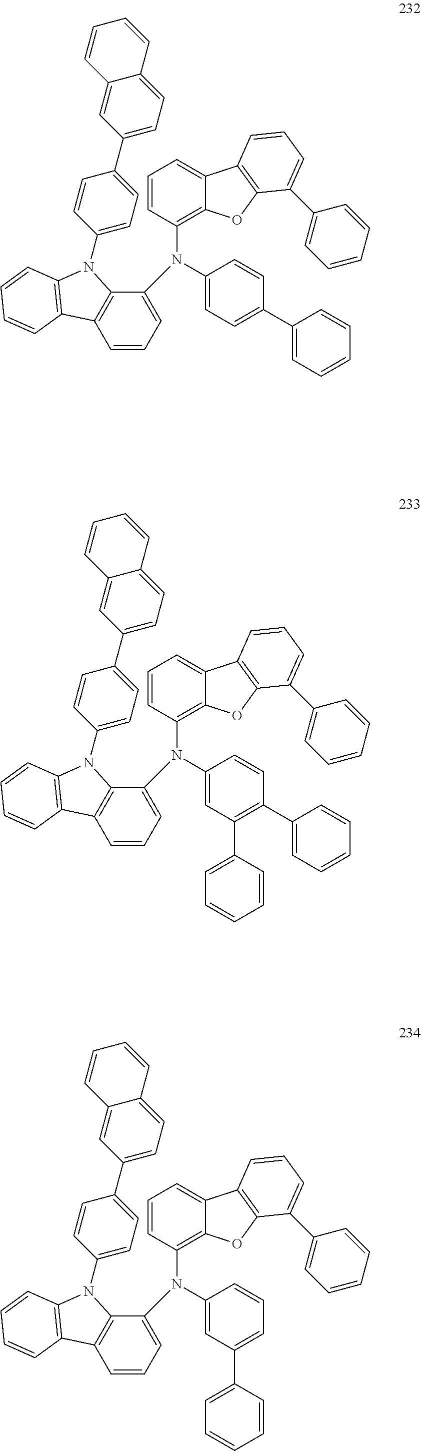 [SMILES: c1ccc(-c2ccc(N(c3cccc4c3oc3c(-c5ccccc5)cccc34)c3cccc4c5ccccc5n(-c5ccc(-c6ccc7ccccc7c6)cc5)c34)cc2)cc1 ; c1ccc(-c2ccc(N(c3cccc4c3oc3c(-c5ccccc5)cccc34)c3cccc4c5ccccc5n(-c5ccc(-c6ccc7ccccc7c6)cc5)c34)cc2-c2ccccc2)cc1 ; c1ccc(-c2cccc(N(c3cccc4c3oc3c(-c5ccccc5)cccc34)c3cccc4c5ccccc5n(-c5ccc(-c6ccc7ccccc7c6)cc5)c34)c2)cc1] 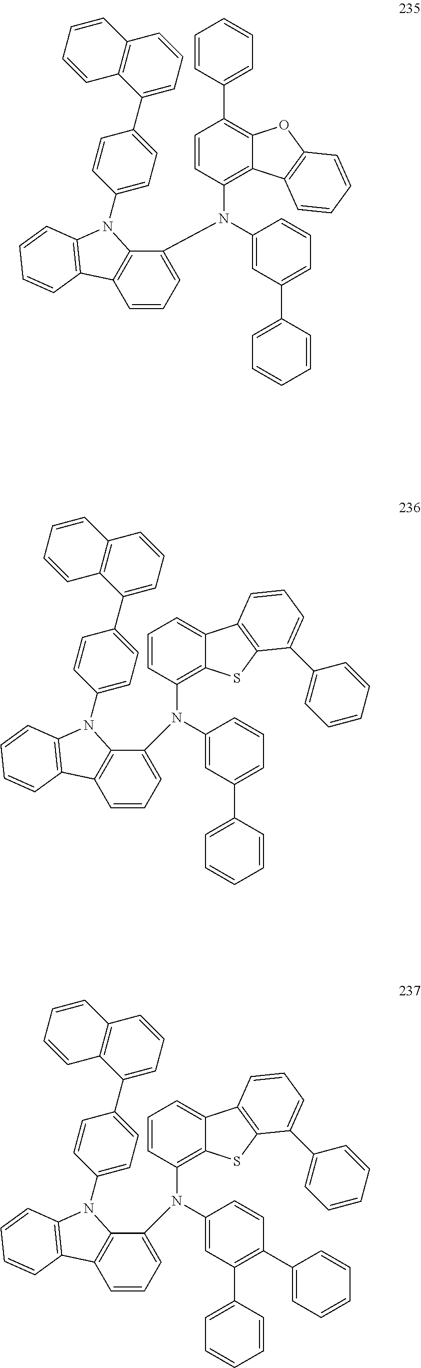 [SMILES: c1ccc(-c2cccc(N(c3ccc(-c4ccccc4)c4oc5ccccc5c34)c3cccc4c5ccccc5n(-c5ccc(-c6cccc7ccccc67)cc5)c34)c2)cc1 ; c1ccc(-c2cccc(N(c3cccc4c3sc3c(-c5ccccc5)cccc34)c3cccc4c5ccccc5n(-c5ccc(-c6cccc7ccccc67)cc5)c34)c2)cc1 ; c1ccc(-c2ccc(N(c3cccc4c3sc3c(-c5ccccc5)cccc34)c3cccc4c5ccccc5n(-c5ccc(-c6cccc7ccccc67)cc5)c34)cc2-c2ccccc2)cc1] 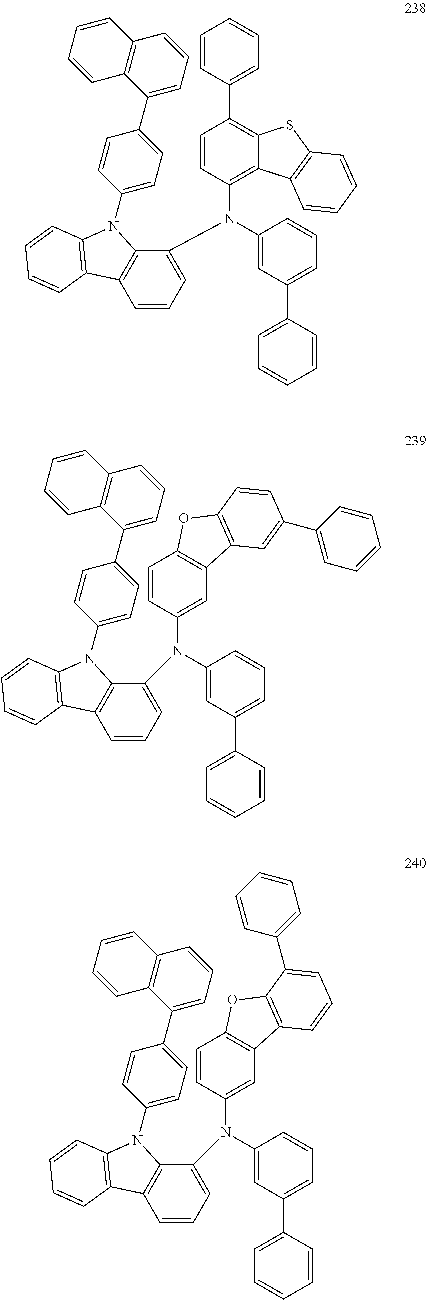 [SMILES: c1ccc(-c2cccc(N(c3ccc(-c4ccccc4)c4sc5ccccc5c34)c3cccc4c5ccccc5n(-c5ccc(-c6cccc7ccccc67)cc5)c34)c2)cc1 ; c1ccc(-c2cccc(N(c3ccc4oc5ccc(-c6ccccc6)cc5c4c3)c3cccc4c5ccccc5n(-c5ccc(-c6cccc7ccccc67)cc5)c34)c2)cc1 ; c1ccc(-c2cccc(N(c3ccc4oc5c(-c6ccccc6)cccc5c4c3)c3cccc4c5ccccc5n(-c5ccc(-c6cccc7ccccc67)cc5)c34)c2)cc1] 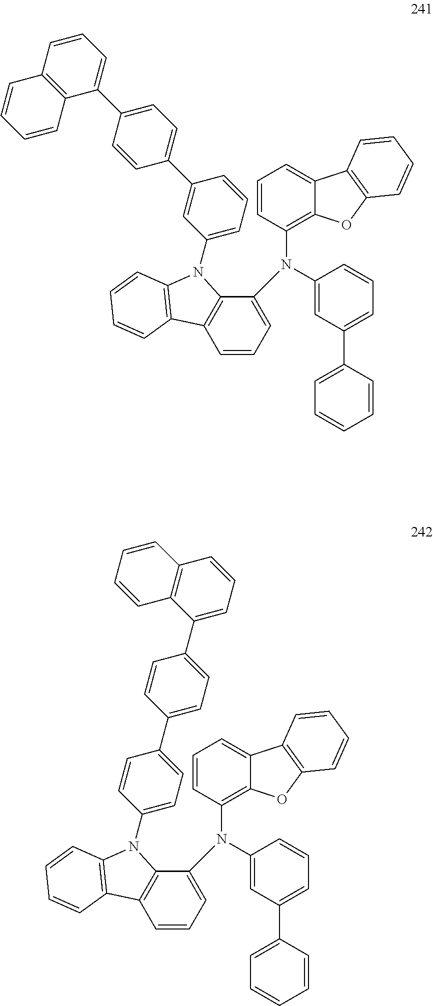 [SMILES: c1ccc(-c2cccc(N(c3cccc4c3oc3ccccc34)c3cccc4c5ccccc5n(-c5cccc(-c6ccc(-c7cccc8ccccc78)cc6)c5)c34)c2)cc1 ; c1ccc(-c2cccc(N(c3cccc4c3oc3ccccc34)c3cccc4c5ccccc5n(-c5ccc(-c6ccc(-c7cccc8ccccc78)cc6)cc5)c34)c2)cc1] 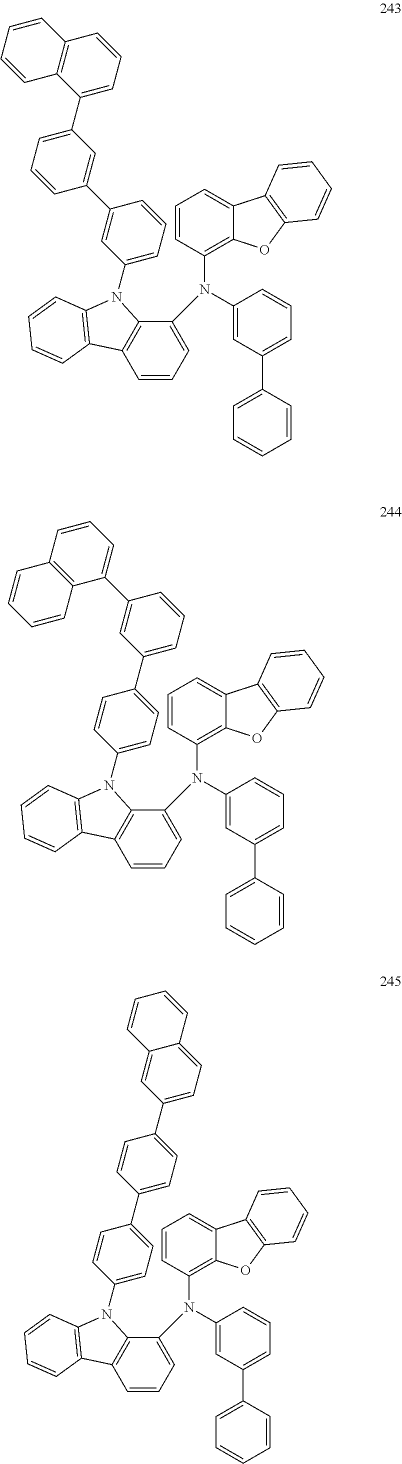 [SMILES: c1ccc(-c2cccc(N(c3cccc4c3oc3ccccc34)c3cccc4c5ccccc5n(-c5cccc(-c6cccc(-c7cccc8ccccc78)c6)c5)c34)c2)cc1 ; c1ccc(-c2cccc(N(c3cccc4c3oc3ccccc34)c3cccc4c5ccccc5n(-c5ccc(-c6cccc(-c7cccc8ccccc78)c6)cc5)c34)c2)cc1 ; c1ccc(-c2cccc(N(c3cccc4c3oc3ccccc34)c3cccc4c5ccccc5n(-c5ccc(-c6ccc(-c7ccc8ccccc8c7)cc6)cc5)c34)c2)cc1] 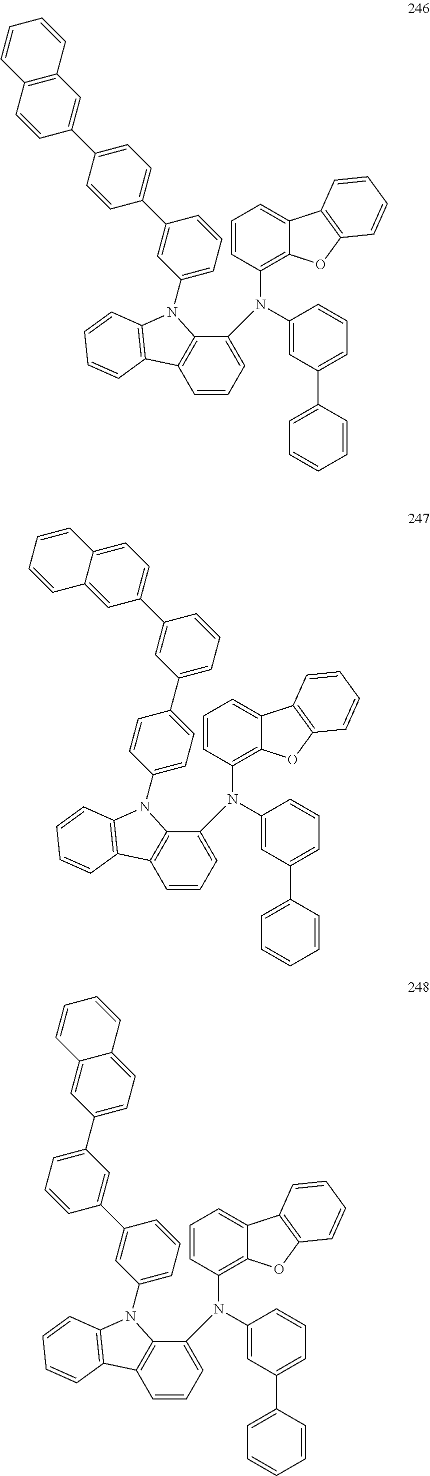 [SMILES: c1ccc(-c2cccc(N(c3cccc4c3oc3ccccc34)c3cccc4c5ccccc5n(-c5cccc(-c6ccc(-c7ccc8ccccc8c7)cc6)c5)c34)c2)cc1 ; c1ccc(-c2cccc(N(c3cccc4c3oc3ccccc34)c3cccc4c5ccccc5n(-c5ccc(-c6cccc(-c7ccc8ccccc8c7)c6)cc5)c34)c2)cc1 ; c1ccc(-c2cccc(N(c3cccc4c3oc3ccccc34)c3cccc4c5ccccc5n(-c5cccc(-c6cccc(-c7ccc8ccccc8c7)c6)c5)c34)c2)cc1] 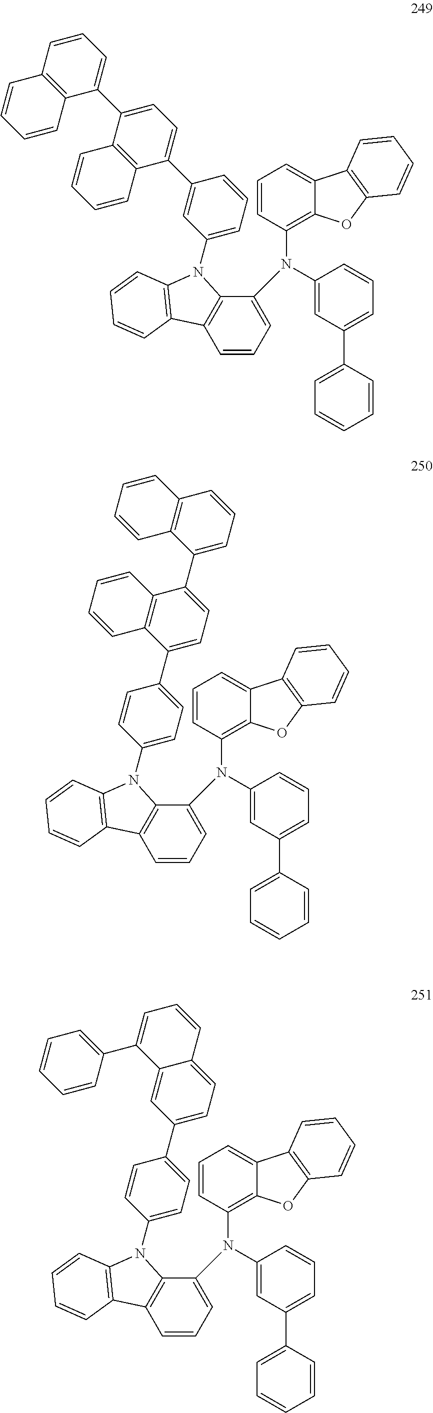 [SMILES: c1ccc(-c2cccc(N(c3cccc4c3oc3ccccc34)c3cccc4c5ccccc5n(-c5cccc(-c6ccc(-c7cccc8ccccc78)c7ccccc67)c5)c34)c2)cc1 ; c1ccc(-c2cccc(N(c3cccc4c3oc3ccccc34)c3cccc4c5ccccc5n(-c5ccc(-c6ccc(-c7cccc8ccccc78)c7ccccc67)cc5)c34)c2)cc1 ; c1ccc(-c2cccc(N(c3cccc4c3oc3ccccc34)c3cccc4c5ccccc5n(-c5ccc(-c6ccc7cccc(-c8ccccc8)c7c6)cc5)c34)c2)cc1] 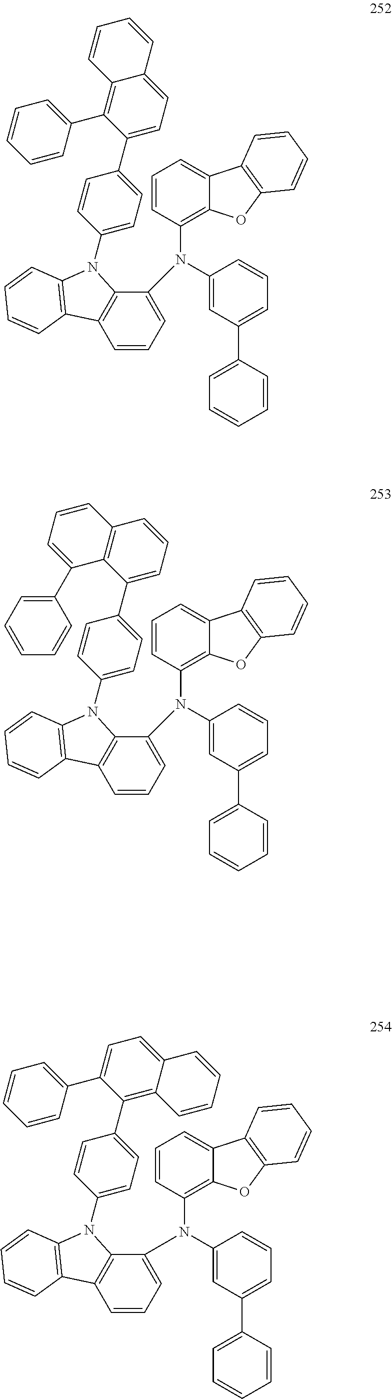 [SMILES: c1ccc(-c2cccc(N(c3cccc4c3oc3ccccc34)c3cccc4c5ccccc5n(-c5ccc(-c6ccc7ccccc7c6-c6ccccc6)cc5)c34)c2)cc1 ; c1ccc(-c2cccc(N(c3cccc4c3oc3ccccc34)c3cccc4c5ccccc5n(-c5ccc(-c6cccc7cccc(-c8ccccc8)c67)cc5)c34)c2)cc1 ; c1ccc(-c2cccc(N(c3cccc4c3oc3ccccc34)c3cccc4c5ccccc5n(-c5ccc(-c6c(-c7ccccc7)ccc7ccccc67)cc5)c34)c2)cc1] 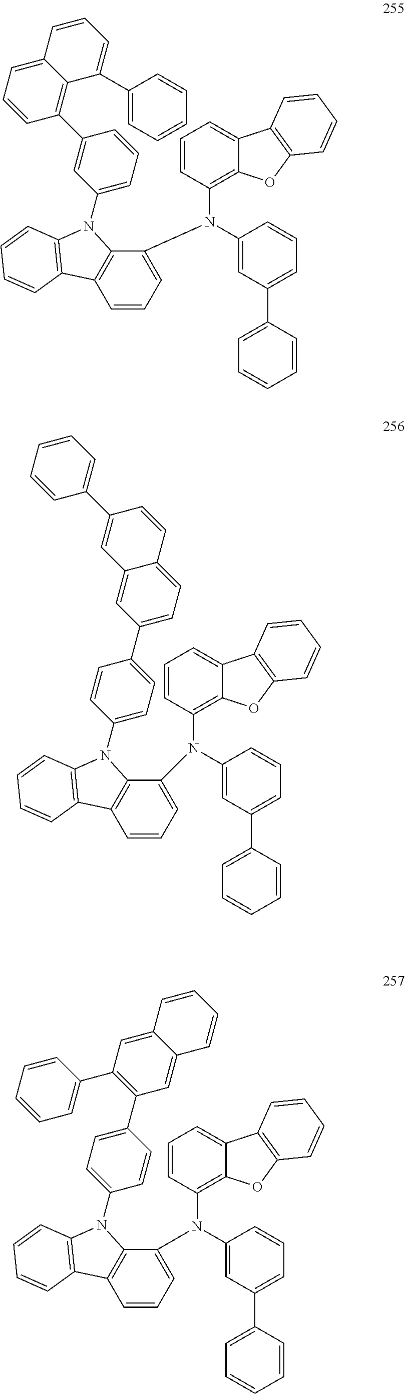 [SMILES: c1ccc(-c2cccc(N(c3cccc4c3oc3ccccc34)c3cccc4c5ccccc5n(-c5cccc(-c6cccc7cccc(-c8ccccc8)c67)c5)c34)c2)cc1 ; c1ccc(-c2cccc(N(c3cccc4c3oc3ccccc34)c3cccc4c5ccccc5n(-c5ccc(-c6ccc7ccc(-c8ccccc8)cc7c6)cc5)c34)c2)cc1 ; c1ccc(-c2cccc(N(c3cccc4c3oc3ccccc34)c3cccc4c5ccccc5n(-c5ccc(-c6cc7ccccc7cc6-c6ccccc6)cc5)c34)c2)cc1] 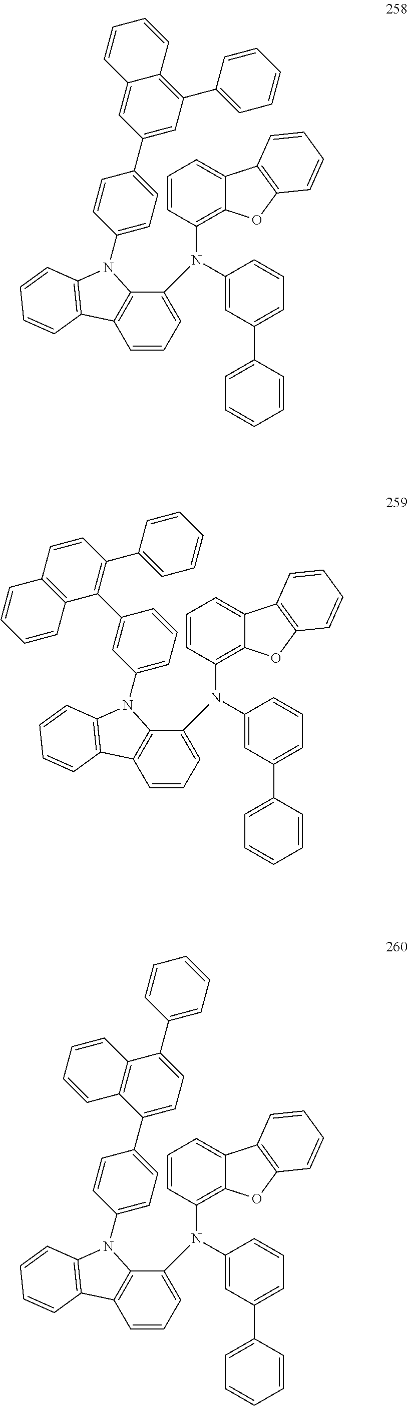 [SMILES: c1ccc(-c2cccc(N(c3cccc4c3oc3ccccc34)c3cccc4c5ccccc5n(-c5ccc(-c6cc(-c7ccccc7)c7ccccc7c6)cc5)c34)c2)cc1 ; c1ccc(-c2cccc(N(c3cccc4c3oc3ccccc34)c3cccc4c5ccccc5n(-c5cccc(-c6c(-c7ccccc7)ccc7ccccc67)c5)c34)c2)cc1 ; c1ccc(-c2cccc(N(c3cccc4c3oc3ccccc34)c3cccc4c5ccccc5n(-c5ccc(-c6ccc(-c7ccccc7)c7ccccc67)cc5)c34)c2)cc1] 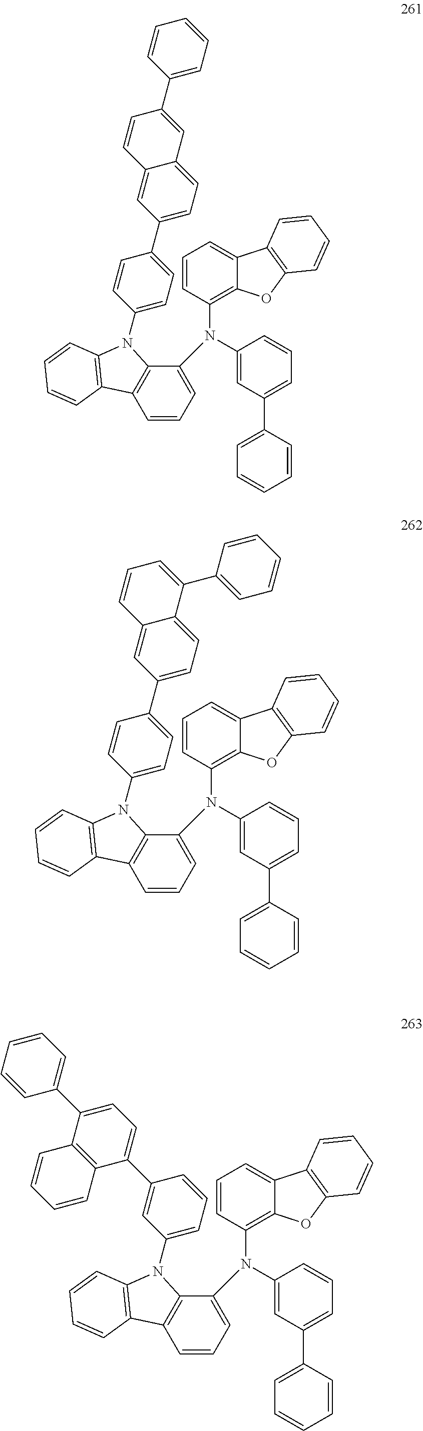 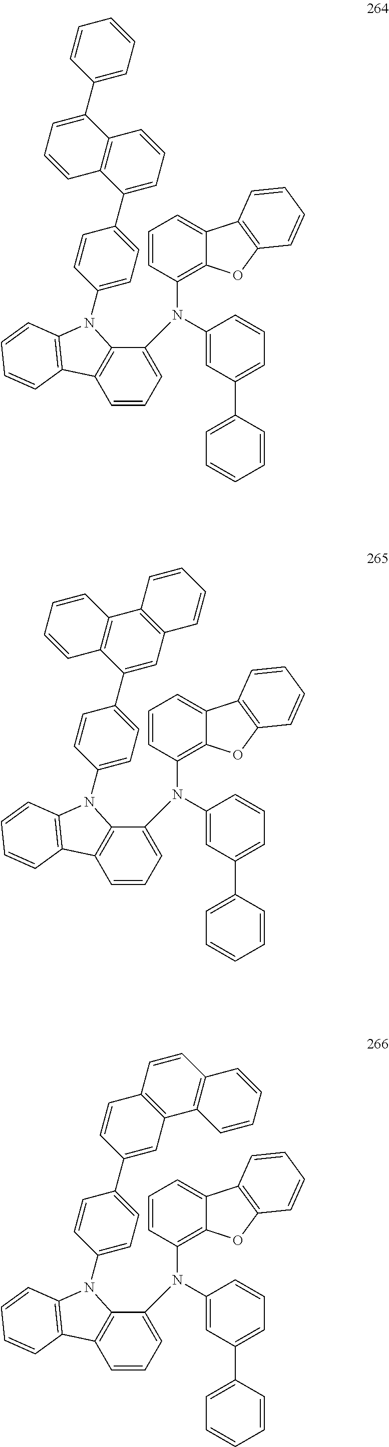 [SMILES: c1ccc(-c2cccc(N(c3cccc4c3oc3ccccc34)c3cccc4c5ccccc5n(-c5ccc(-c6cccc7c(-c8ccccc8)cccc67)cc5)c34)c2)cc1 ; c1ccc(-c2cccc(N(c3cccc4c3oc3ccccc34)c3cccc4c5ccccc5n(-c5ccc(-c6cc7ccccc7c7ccccc67)cc5)c34)c2)cc1 ; c1ccc(-c2cccc(N(c3cccc4c3oc3ccccc34)c3cccc4c5ccccc5n(-c5ccc(-c6ccc7ccc8ccccc8c7c6)cc5)c34)c2)cc1] 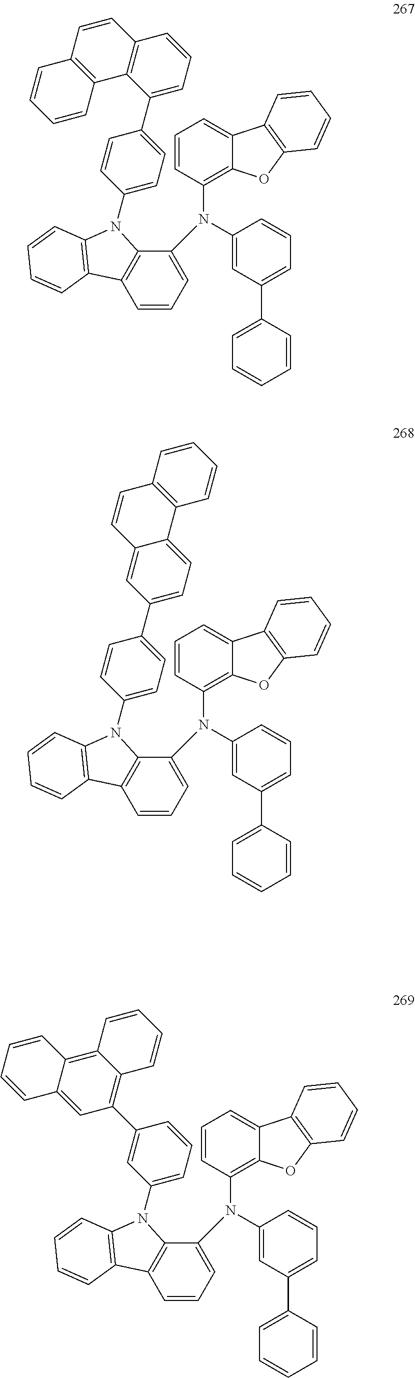 [SMILES: c1ccc(-c2cccc(N(c3cccc4c3oc3ccccc34)c3cccc4c5ccccc5n(-c5ccc(-c6cccc7ccc8ccccc8c67)cc5)c34)c2)cc1 ; c1ccc(-c2cccc(N(c3cccc4c3oc3ccccc34)c3cccc4c5ccccc5n(-c5ccc(-c6ccc7c(ccc8ccccc87)c6)cc5)c34)c2)cc1 ; c1ccc(-c2cccc(N(c3cccc4c3oc3ccccc34)c3cccc4c5ccccc5n(-c5cccc(-c6cc7ccccc7c7ccccc67)c5)c34)c2)cc1] 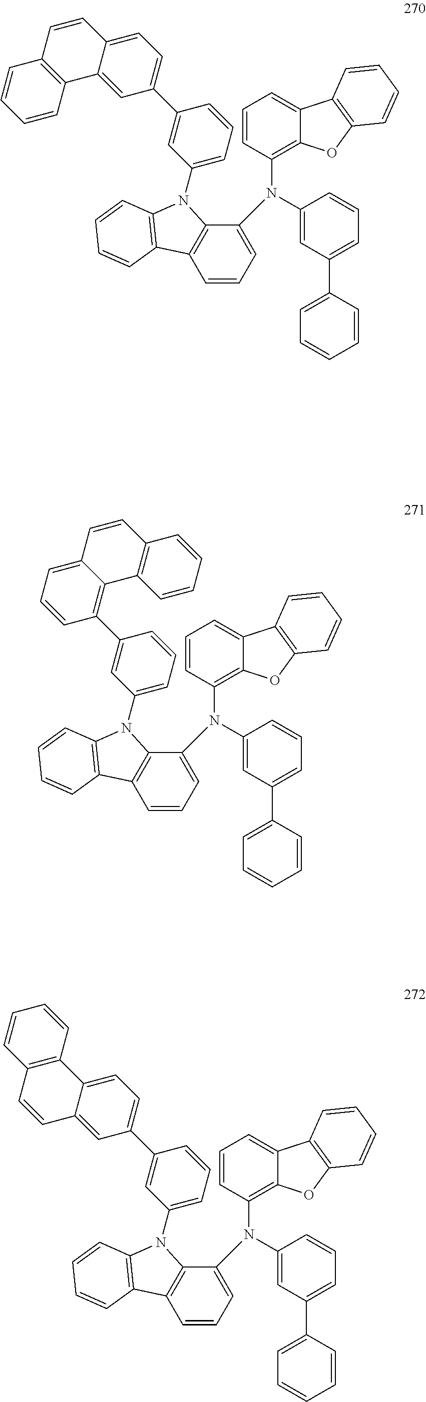 [SMILES: c1ccc(-c2cccc(N(c3cccc4c3oc3ccccc34)c3cccc4c5ccccc5n(-c5cccc(-c6ccc7ccc8ccccc8c7c6)c5)c34)c2)cc1 ; c1ccc(-c2cccc(N(c3cccc4c3oc3ccccc34)c3cccc4c5ccccc5n(-c5cccc(-c6cccc7ccc8ccccc8c67)c5)c34)c2)cc1 ; c1ccc(-c2cccc(N(c3cccc4c3oc3ccccc34)c3cccc4c5ccccc5n(-c5cccc(-c6ccc7c(ccc8ccccc87)c6)c5)c34)c2)cc1] 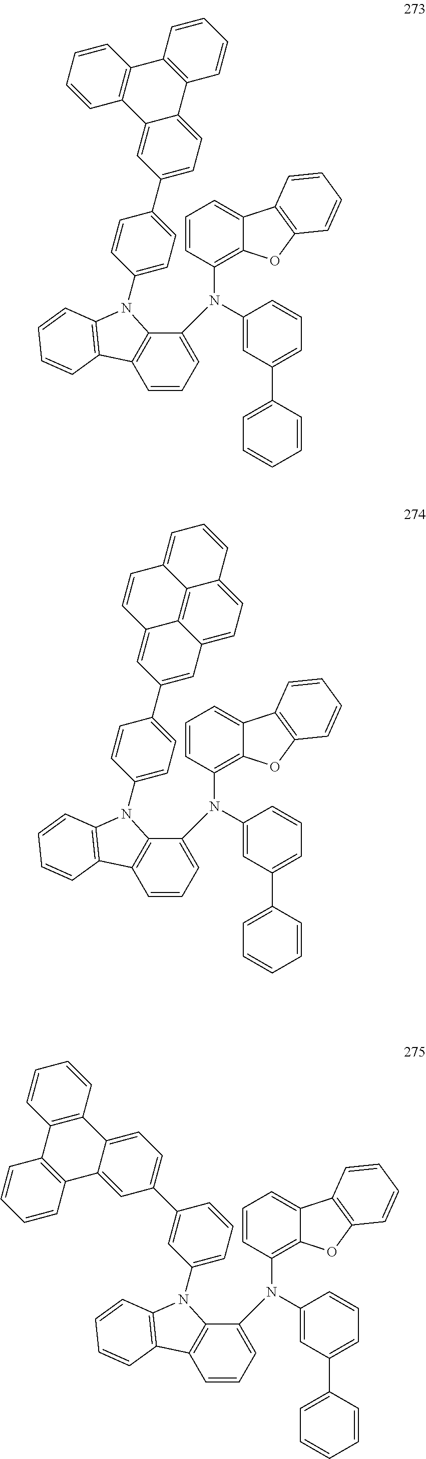 [SMILES: c1ccc(-c2cccc(N(c3cccc4c3oc3ccccc34)c3cccc4c5ccccc5n(-c5ccc(-c6ccc7c8ccccc8c8ccccc8c7c6)cc5)c34)c2)cc1 ; c1ccc(-c2cccc(N(c3cccc4c3oc3ccccc34)c3cccc4c5ccccc5n(-c5ccc(-c6cc7ccc8cccc9ccc(c6)c7c89)cc5)c34)c2)cc1 ; c1ccc(-c2cccc(N(c3cccc4c3oc3ccccc34)c3cccc4c5ccccc5n(-c5cccc(-c6ccc7c8ccccc8c8ccccc8c7c6)c5)c34)c2)cc1] 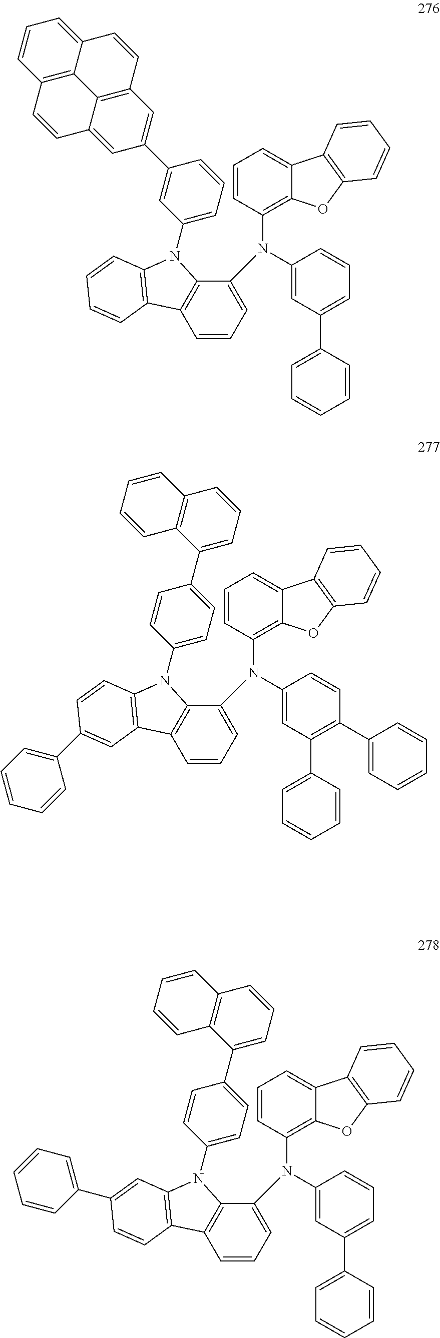 [SMILES: c1ccc(-c2cccc(N(c3cccc4c3oc3ccccc34)c3cccc4c5ccccc5n(-c5cccc(-c6cc7ccc8cccc9ccc(c6)c7c89)c5)c34)c2)cc1 ; c1ccc(-c2ccc3c(c2)c2cccc(N(c4ccc(-c5ccccc5)c(-c5ccccc5)c4)c4cccc5c4oc4ccccc45)c2n3-c2ccc(-c3cccc4ccccc34)cc2)cc1 ; c1ccc(-c2cccc(N(c3cccc4c3oc3ccccc34)c3cccc4c5ccc(-c6ccccc6)cc5n(-c5ccc(-c6cccc7ccccc67)cc5)c34)c2)cc1] 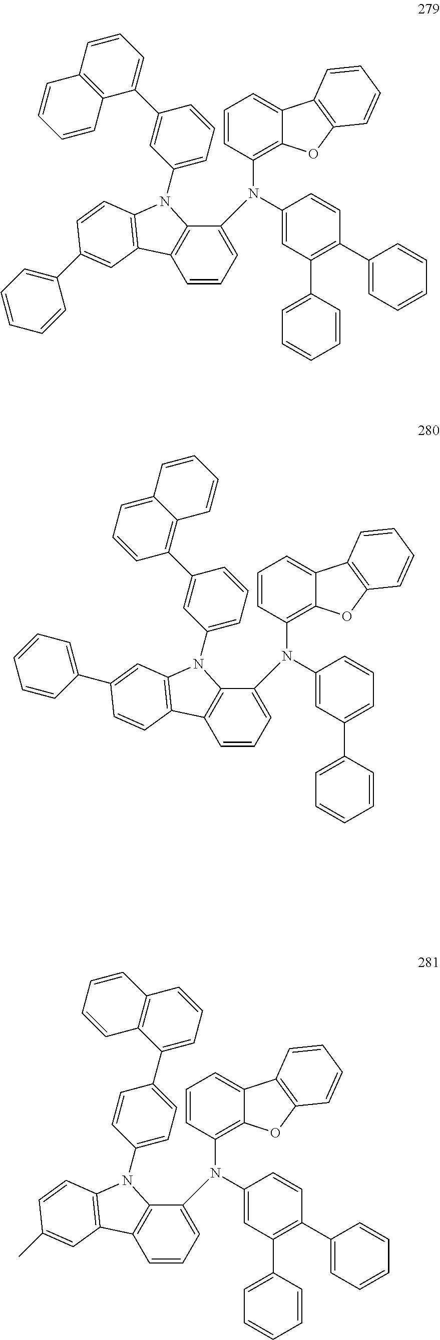 [SMILES: c1ccc(-c2ccc3c(c2)c2cccc(N(c4ccc(-c5ccccc5)c(-c5ccccc5)c4)c4cccc5c4oc4ccccc45)c2n3-c2cccc(-c3cccc4ccccc34)c2)cc1 ; c1ccc(-c2cccc(N(c3cccc4c3oc3ccccc34)c3cccc4c5ccc(-c6ccccc6)cc5n(-c5cccc(-c6cccc7ccccc67)c5)c34)c2)cc1 ; Cc1ccc2c(c1)c1cccc(N(c3ccc(-c4ccccc4)c(-c4ccccc4)c3)c3cccc4c3oc3ccccc34)c1n2-c1ccc(-c2cccc3ccccc23)cc1] 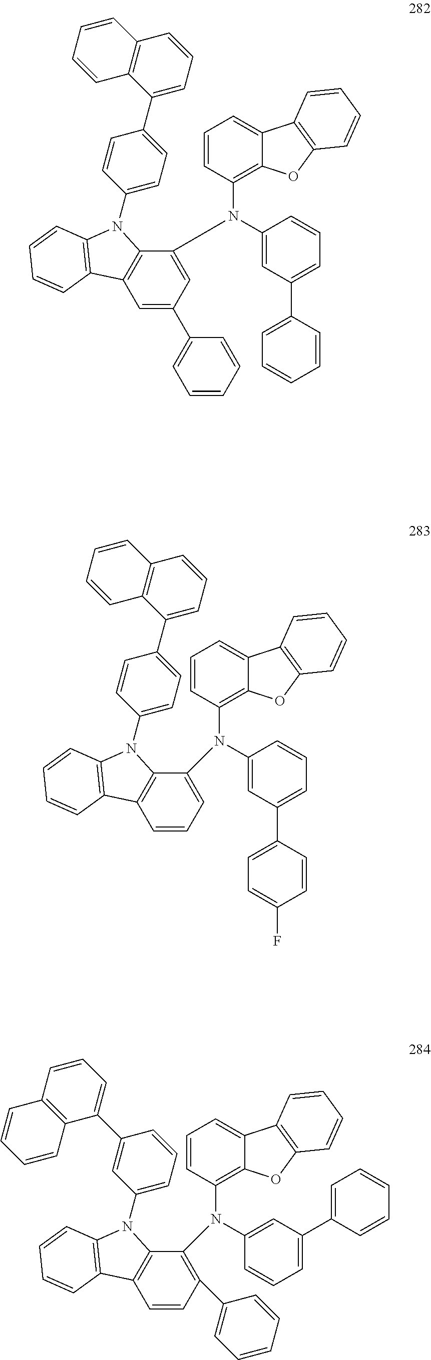 [SMILES: c1ccc(-c2cccc(N(c3cccc4c3oc3ccccc34)c3cc(-c4ccccc4)cc4c5ccccc5n(-c5ccc(-c6cccc7ccccc67)cc5)c34)c2)cc1 ; Fc1ccc(-c2cccc(N(c3cccc4c3oc3ccccc34)c3cccc4c5ccccc5n(-c5ccc(-c6cccc7ccccc67)cc5)c34)c2)cc1 ; c1ccc(-c2cccc(N(c3cccc4c3oc3ccccc34)c3c(-c4ccccc4)ccc4c5ccccc5n(-c5cccc(-c6cccc7ccccc67)c5)c34)c2)cc1] 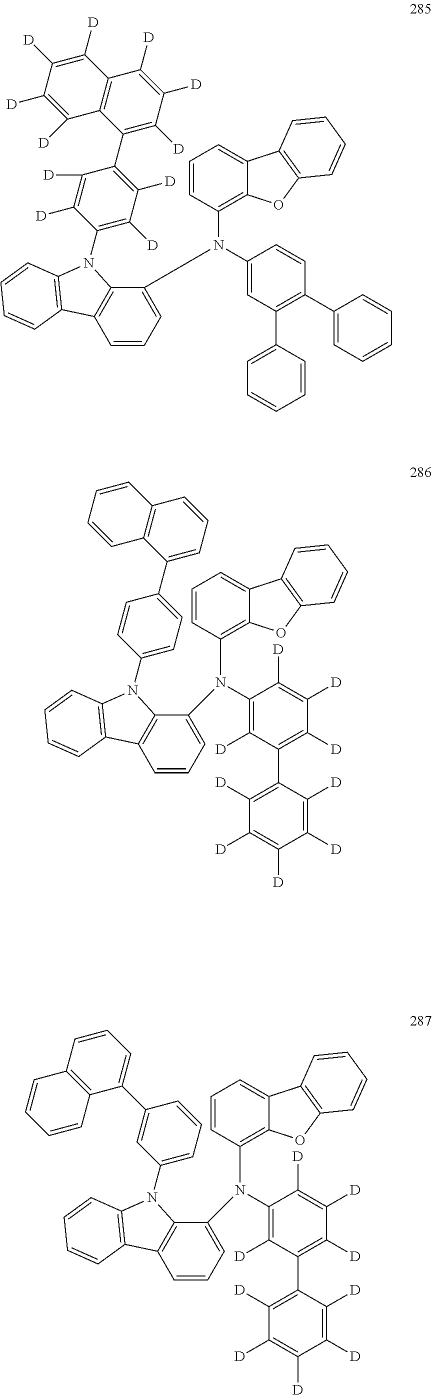 [SMILES: [2H]c1c([2H])c(-n2c3ccccc3c3cccc(N(c4ccc(-c5ccccc5)c(-c5ccccc5)c4)c4cccc5c4oc4ccccc45)c32)c([2H])c([2H])c1-c1c([2H])c([2H])c([2H])c2c([2H])c([2H])c([2H])c([2H])c12 ; [2H]c1c([2H])c([2H])c(-c2c([2H])c([2H])c([2H])c(N(c3cccc4c3oc3ccccc34)c3cccc4c5ccccc5n(-c5ccc(-c6cccc7ccccc67)cc5)c34)c2[2H])c([2H])c1[2H] ; [2H]c1c([2H])c([2H])c(-c2c([2H])c([2H])c([2H])c(N(c3cccc4c3oc3ccccc34)c3cccc4c5ccccc5n(-c5cccc(-c6cccc7ccccc67)c5)c34)c2[2H])c([2H])c1[2H]] 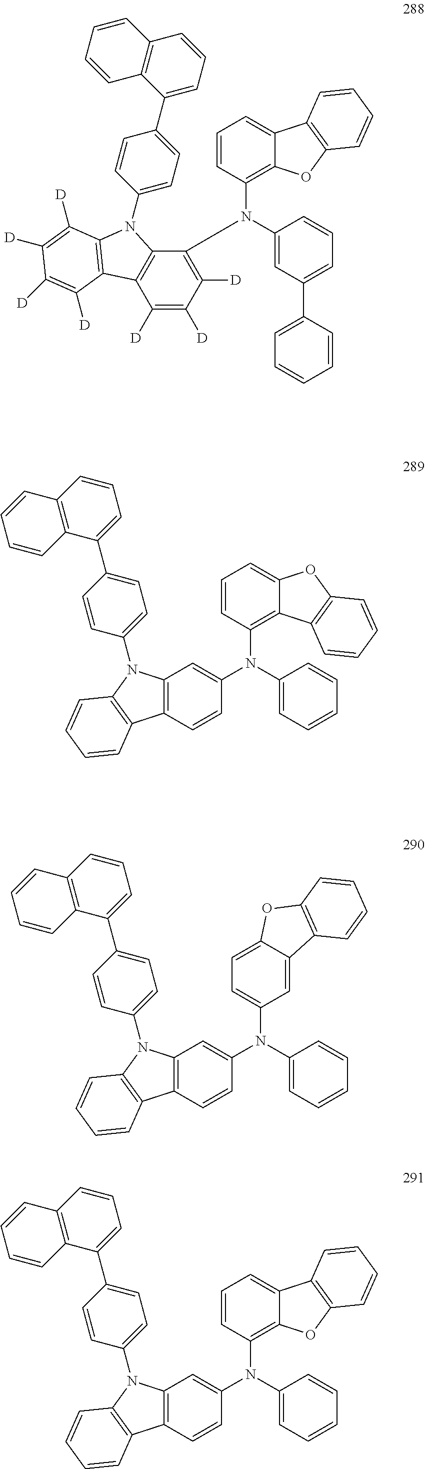 [SMILES: [2H]c1c([2H])c([2H])c2c(c1[2H])c1c([2H])c([2H])c([2H])c(N(c3cccc(-c4ccccc4)c3)c3cccc4c3oc3ccccc34)c1n2-c1ccc(-c2cccc3ccccc23)cc1 ; c1ccc(N(c2ccc3c4ccccc4n(-c4ccc(-c5cccc6ccccc56)cc4)c3c2)c2cccc3oc4ccccc4c23)cc1 ; c1ccc(N(c2ccc3oc4ccccc4c3c2)c2ccc3c4ccccc4n(-c4ccc(-c5cccc6ccccc56)cc4)c3c2)cc1 ; c1ccc(N(c2ccc3c4ccccc4n(-c4ccc(-c5cccc6ccccc56)cc4)c3c2)c2cccc3c2oc2ccccc23)cc1] 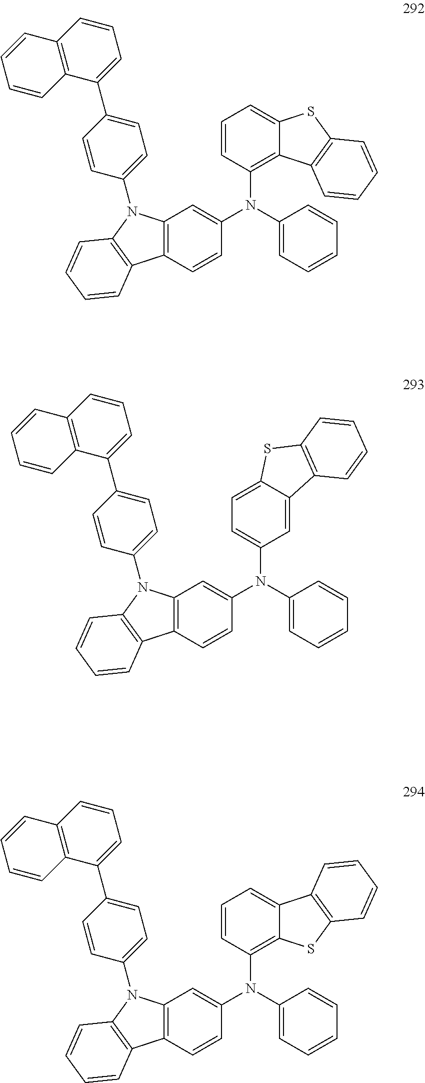 [SMILES: c1ccc(N(c2ccc3c4ccccc4n(-c4ccc(-c5cccc6ccccc56)cc4)c3c2)c2cccc3sc4ccccc4c23)cc1 ; c1ccc(N(c2ccc3sc4ccccc4c3c2)c2ccc3c4ccccc4n(-c4ccc(-c5cccc6ccccc56)cc4)c3c2)cc1 ; c1ccc(N(c2ccc3c4ccccc4n(-c4ccc(-c5cccc6ccccc56)cc4)c3c2)c2cccc3c2sc2ccccc23)cc1] 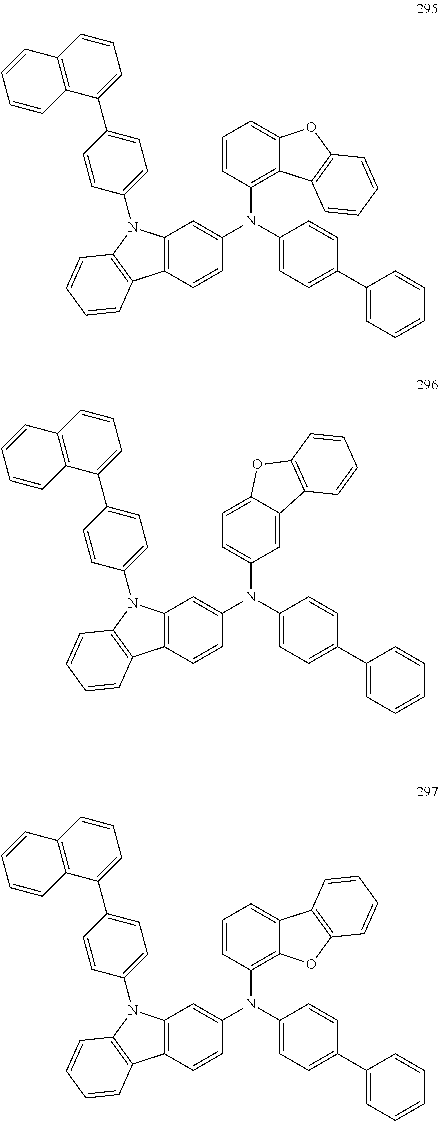 [SMILES: c1ccc(-c2ccc(N(c3ccc4c5ccccc5n(-c5ccc(-c6cccc7ccccc67)cc5)c4c3)c3cccc4oc5ccccc5c34)cc2)cc1 ; c1ccc(-c2ccc(N(c3ccc4oc5ccccc5c4c3)c3ccc4c5ccccc5n(-c5ccc(-c6cccc7ccccc67)cc5)c4c3)cc2)cc1 ; c1ccc(-c2ccc(N(c3ccc4c5ccccc5n(-c5ccc(-c6cccc7ccccc67)cc5)c4c3)c3cccc4c3oc3ccccc34)cc2)cc1] 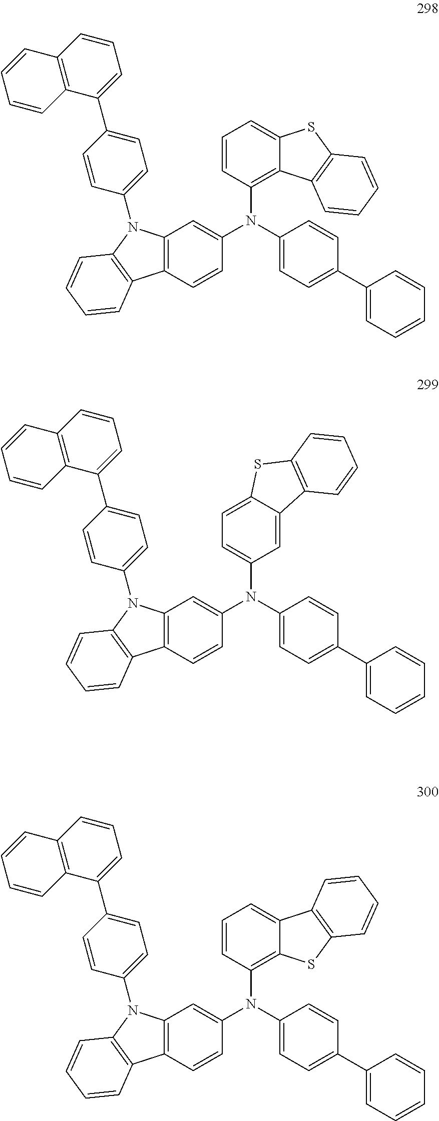 [SMILES: c1ccc(-c2ccc(N(c3ccc4c5ccccc5n(-c5ccc(-c6cccc7ccccc67)cc5)c4c3)c3cccc4sc5ccccc5c34)cc2)cc1 ; c1ccc(-c2ccc(N(c3ccc4sc5ccccc5c4c3)c3ccc4c5ccccc5n(-c5ccc(-c6cccc7ccccc67)cc5)c4c3)cc2)cc1 ; c1ccc(-c2ccc(N(c3ccc4c5ccccc5n(-c5ccc(-c6cccc7ccccc67)cc5)c4c3)c3cccc4c3sc3ccccc34)cc2)cc1] 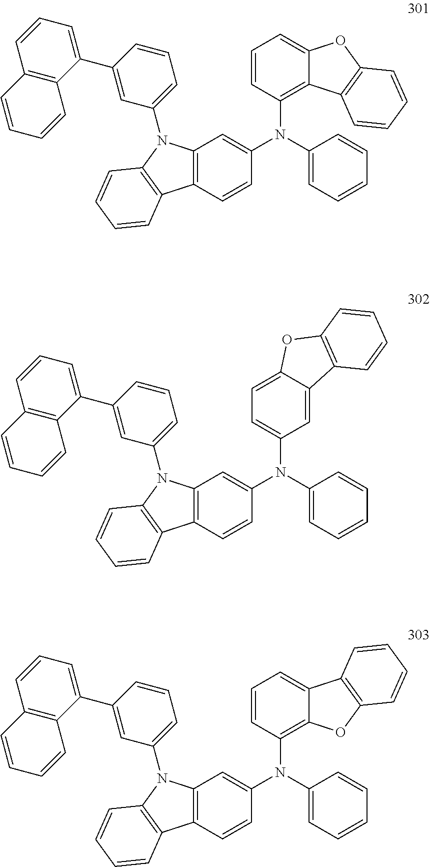 [SMILES: c1ccc(N(c2ccc3c4ccccc4n(-c4cccc(-c5cccc6ccccc56)c4)c3c2)c2cccc3oc4ccccc4c23)cc1 ; c1ccc(N(c2ccc3oc4ccccc4c3c2)c2ccc3c4ccccc4n(-c4cccc(-c5cccc6ccccc56)c4)c3c2)cc1 ; c1ccc(N(c2ccc3c4ccccc4n(-c4cccc(-c5cccc6ccccc56)c4)c3c2)c2cccc3c2oc2ccccc23)cc1] 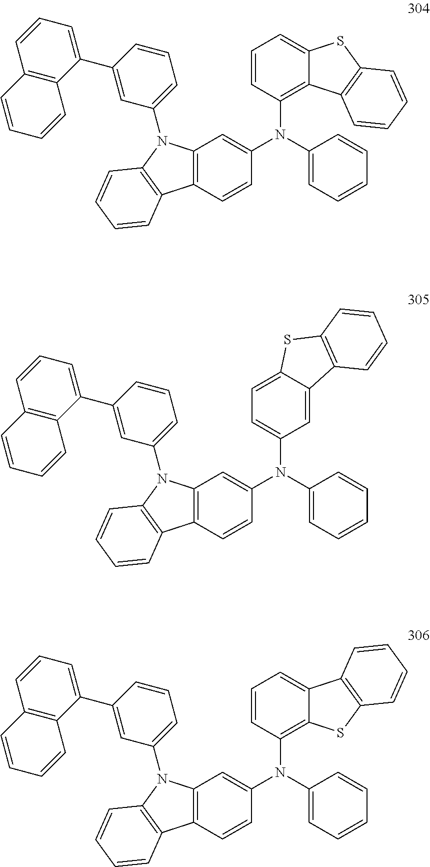 [SMILES: c1ccc(N(c2ccc3c4ccccc4n(-c4cccc(-c5cccc6ccccc56)c4)c3c2)c2cccc3sc4ccccc4c23)cc1 ; c1ccc(N(c2ccc3sc4ccccc4c3c2)c2ccc3c4ccccc4n(-c4cccc(-c5cccc6ccccc56)c4)c3c2)cc1 ; c1ccc(N(c2ccc3c4ccccc4n(-c4cccc(-c5cccc6ccccc56)c4)c3c2)c2cccc3c2sc2ccccc23)cc1] 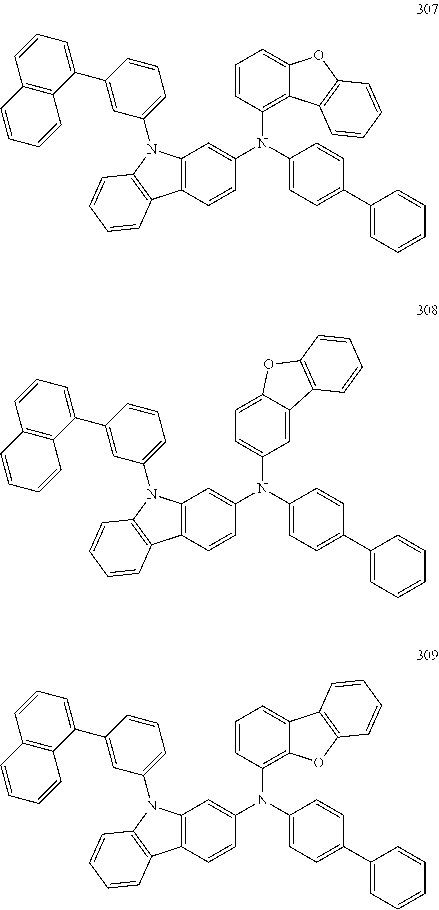 [SMILES: c1ccc(-c2ccc(N(c3ccc4c5ccccc5n(-c5cccc(-c6cccc7ccccc67)c5)c4c3)c3cccc4oc5ccccc5c34)cc2)cc1 ; c1ccc(-c2ccc(N(c3ccc4oc5ccccc5c4c3)c3ccc4c5ccccc5n(-c5cccc(-c6cccc7ccccc67)c5)c4c3)cc2)cc1 ; c1ccc(-c2ccc(N(c3ccc4c5ccccc5n(-c5cccc(-c6cccc7ccccc67)c5)c4c3)c3cccc4c3oc3ccccc34)cc2)cc1] 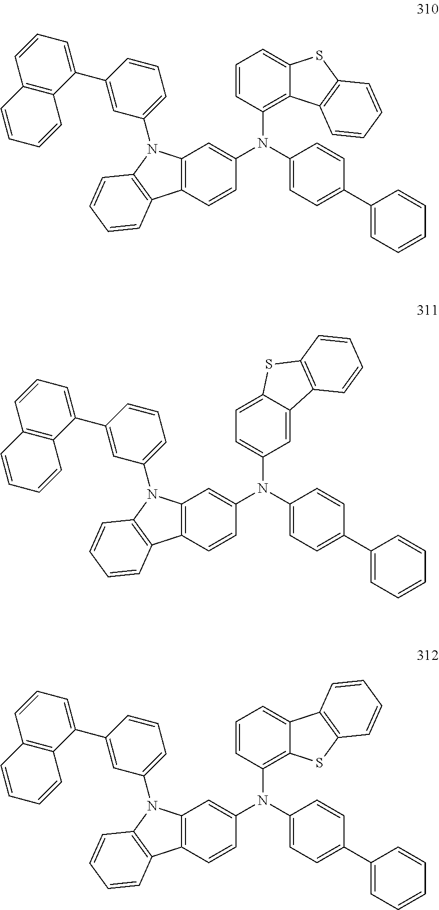 [SMILES: c1ccc(-c2ccc(N(c3ccc4c5ccccc5n(-c5cccc(-c6cccc7ccccc67)c5)c4c3)c3cccc4sc5ccccc5c34)cc2)cc1 ; c1ccc(-c2ccc(N(c3ccc4sc5ccccc5c4c3)c3ccc4c5ccccc5n(-c5cccc(-c6cccc7ccccc67)c5)c4c3)cc2)cc1 ; c1ccc(-c2ccc(N(c3ccc4c5ccccc5n(-c5cccc(-c6cccc7ccccc67)c5)c4c3)c3cccc4c3sc3ccccc34)cc2)cc1] 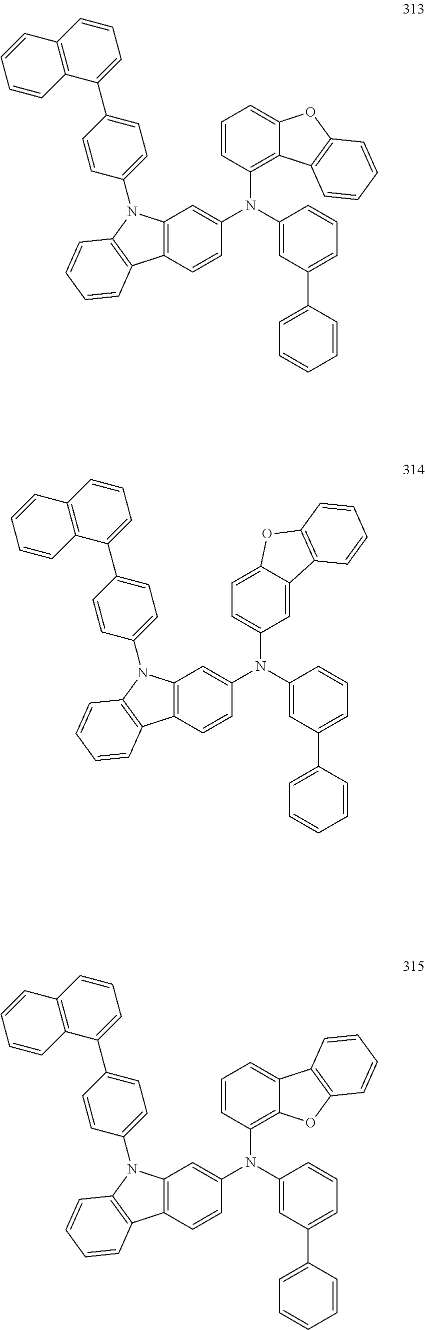 [SMILES: c1ccc(-c2cccc(N(c3ccc4c5ccccc5n(-c5ccc(-c6cccc7ccccc67)cc5)c4c3)c3cccc4oc5ccccc5c34)c2)cc1 ; c1ccc(-c2cccc(N(c3ccc4oc5ccccc5c4c3)c3ccc4c5ccccc5n(-c5ccc(-c6cccc7ccccc67)cc5)c4c3)c2)cc1 ; c1ccc(-c2cccc(N(c3ccc4c5ccccc5n(-c5ccc(-c6cccc7ccccc67)cc5)c4c3)c3cccc4c3oc3ccccc34)c2)cc1] 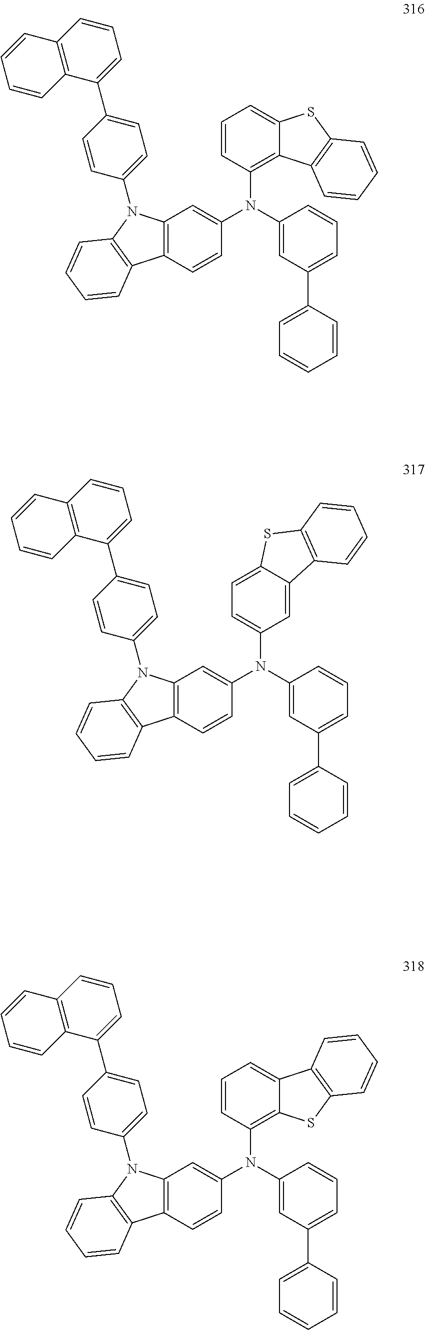 [SMILES: c1ccc(-c2cccc(N(c3ccc4c5ccccc5n(-c5ccc(-c6cccc7ccccc67)cc5)c4c3)c3cccc4sc5ccccc5c34)c2)cc1 ; c1ccc(-c2cccc(N(c3ccc4sc5ccccc5c4c3)c3ccc4c5ccccc5n(-c5ccc(-c6cccc7ccccc67)cc5)c4c3)c2)cc1 ; c1ccc(-c2cccc(N(c3ccc4c5ccccc5n(-c5ccc(-c6cccc7ccccc67)cc5)c4c3)c3cccc4c3sc3ccccc34)c2)cc1] 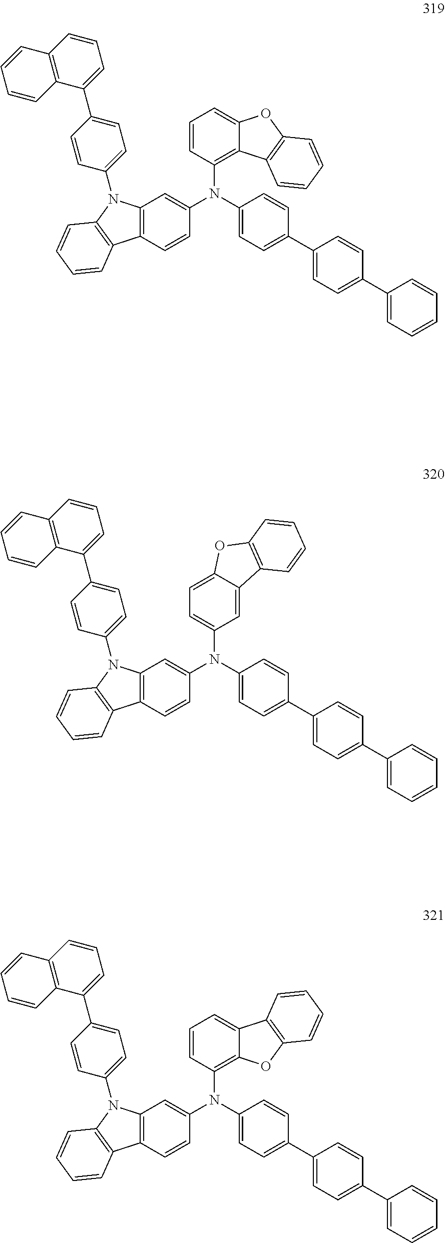 [SMILES: c1ccc(-c2ccc(-c3ccc(N(c4ccc5c6ccccc6n(-c6ccc(-c7cccc8ccccc78)cc6)c5c4)c4cccc5oc6ccccc6c45)cc3)cc2)cc1 ; c1ccc(-c2ccc(-c3ccc(N(c4ccc5oc6ccccc6c5c4)c4ccc5c6ccccc6n(-c6ccc(-c7cccc8ccccc78)cc6)c5c4)cc3)cc2)cc1 ; c1ccc(-c2ccc(-c3ccc(N(c4ccc5c6ccccc6n(-c6ccc(-c7cccc8ccccc78)cc6)c5c4)c4cccc5c4oc4ccccc45)cc3)cc2)cc1] 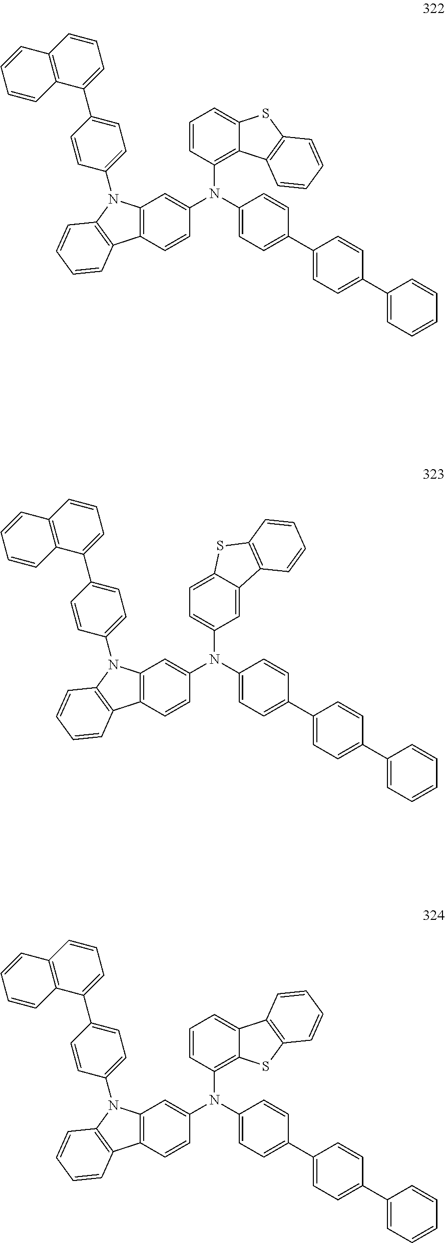 [SMILES: c1ccc(-c2ccc(-c3ccc(N(c4ccc5c6ccccc6n(-c6ccc(-c7cccc8ccccc78)cc6)c5c4)c4cccc5sc6ccccc6c45)cc3)cc2)cc1 ; c1ccc(-c2ccc(-c3ccc(N(c4ccc5sc6ccccc6c5c4)c4ccc5c6ccccc6n(-c6ccc(-c7cccc8ccccc78)cc6)c5c4)cc3)cc2)cc1 ; c1ccc(-c2ccc(-c3ccc(N(c4ccc5c6ccccc6n(-c6ccc(-c7cccc8ccccc78)cc6)c5c4)c4cccc5c4sc4ccccc45)cc3)cc2)cc1] 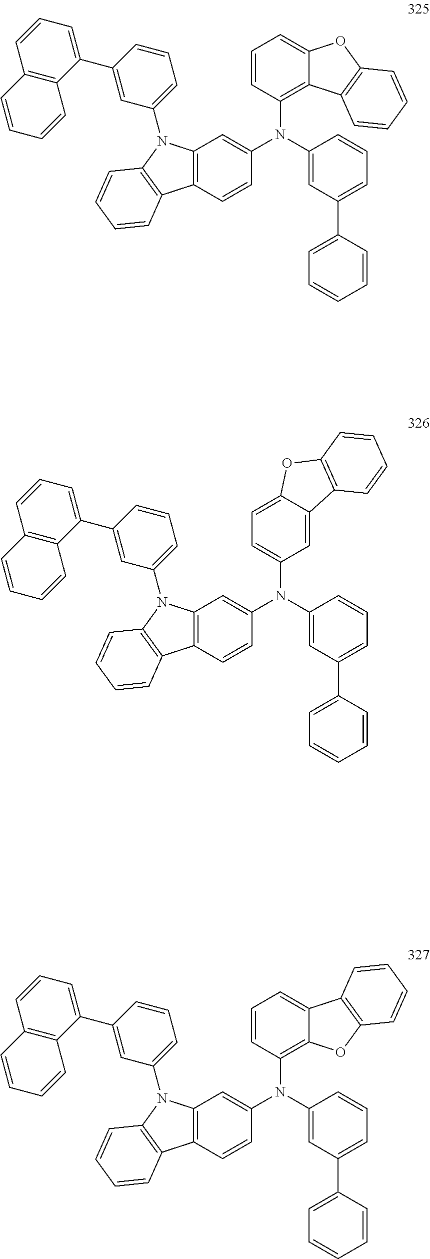 [SMILES: c1ccc(-c2cccc(N(c3ccc4c5ccccc5n(-c5cccc(-c6cccc7ccccc67)c5)c4c3)c3cccc4oc5ccccc5c34)c2)cc1 ; c1ccc(-c2cccc(N(c3ccc4oc5ccccc5c4c3)c3ccc4c5ccccc5n(-c5cccc(-c6cccc7ccccc67)c5)c4c3)c2)cc1 ; c1ccc(-c2cccc(N(c3ccc4c5ccccc5n(-c5cccc(-c6cccc7ccccc67)c5)c4c3)c3cccc4c3oc3ccccc34)c2)cc1] 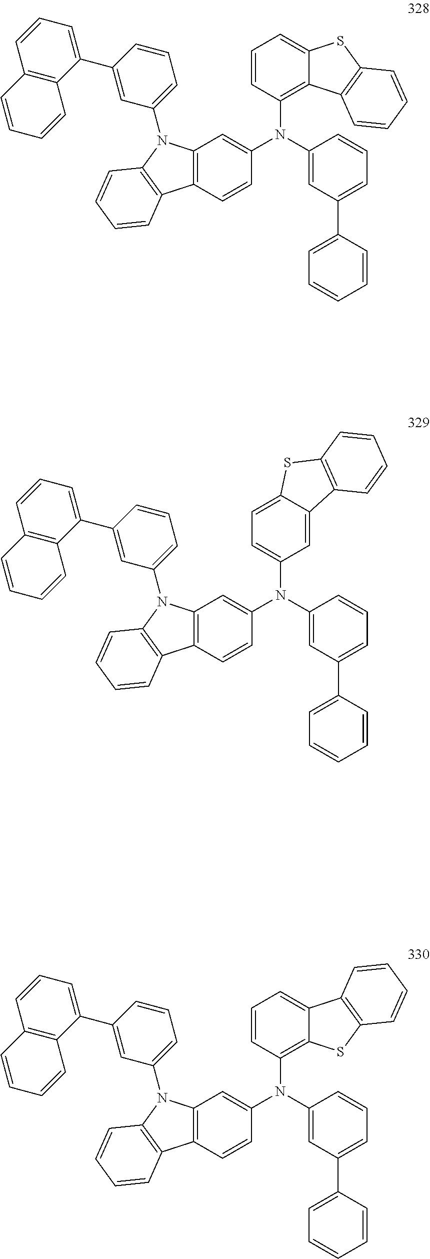 [SMILES: c1ccc(-c2cccc(N(c3ccc4c5ccccc5n(-c5cccc(-c6cccc7ccccc67)c5)c4c3)c3cccc4sc5ccccc5c34)c2)cc1 ; c1ccc(-c2cccc(N(c3ccc4sc5ccccc5c4c3)c3ccc4c5ccccc5n(-c5cccc(-c6cccc7ccccc67)c5)c4c3)c2)cc1 ; c1ccc(-c2cccc(N(c3ccc4c5ccccc5n(-c5cccc(-c6cccc7ccccc67)c5)c4c3)c3cccc4c3sc3ccccc34)c2)cc1] 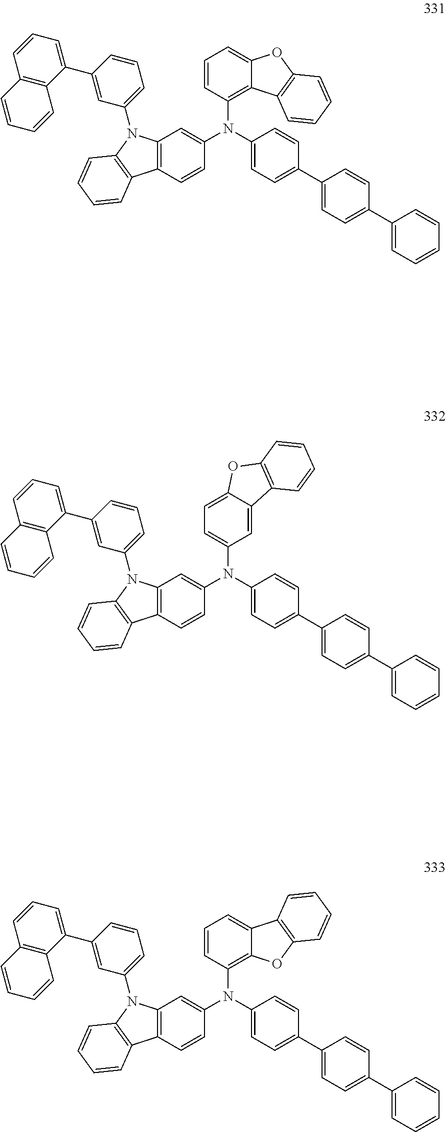 [SMILES: c1ccc(-c2ccc(-c3ccc(N(c4ccc5c6ccccc6n(-c6cccc(-c7cccc8ccccc78)c6)c5c4)c4cccc5oc6ccccc6c45)cc3)cc2)cc1 ; c1ccc(-c2ccc(-c3ccc(N(c4ccc5oc6ccccc6c5c4)c4ccc5c6ccccc6n(-c6cccc(-c7cccc8ccccc78)c6)c5c4)cc3)cc2)cc1 ; c1ccc(-c2ccc(-c3ccc(N(c4ccc5c6ccccc6n(-c6cccc(-c7cccc8ccccc78)c6)c5c4)c4cccc5c4oc4ccccc45)cc3)cc2)cc1] 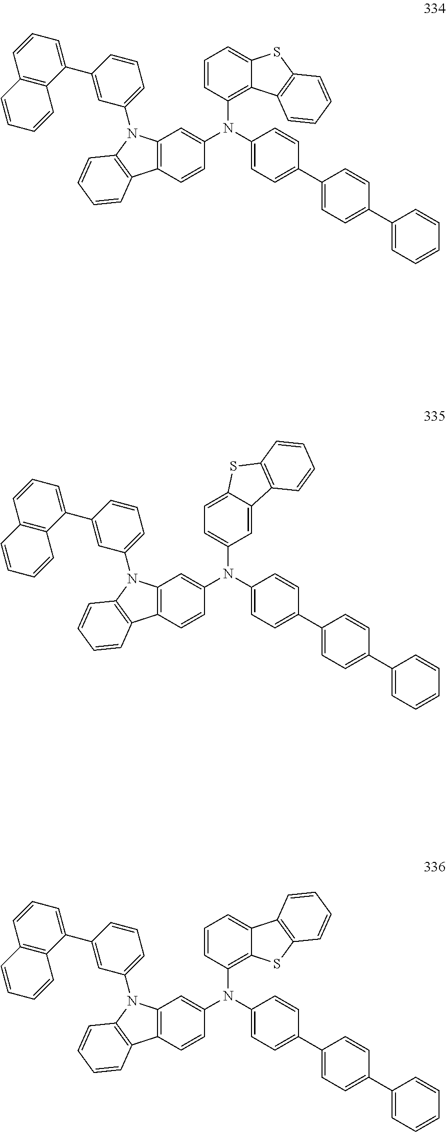 [SMILES: c1ccc(-c2ccc(-c3ccc(N(c4ccc5c6ccccc6n(-c6cccc(-c7cccc8ccccc78)c6)c5c4)c4cccc5sc6ccccc6c45)cc3)cc2)cc1 ; c1ccc(-c2ccc(-c3ccc(N(c4ccc5sc6ccccc6c5c4)c4ccc5c6ccccc6n(-c6cccc(-c7cccc8ccccc78)c6)c5c4)cc3)cc2)cc1 ; c1ccc(-c2ccc(-c3ccc(N(c4ccc5c6ccccc6n(-c6cccc(-c7cccc8ccccc78)c6)c5c4)c4cccc5c4sc4ccccc45)cc3)cc2)cc1] 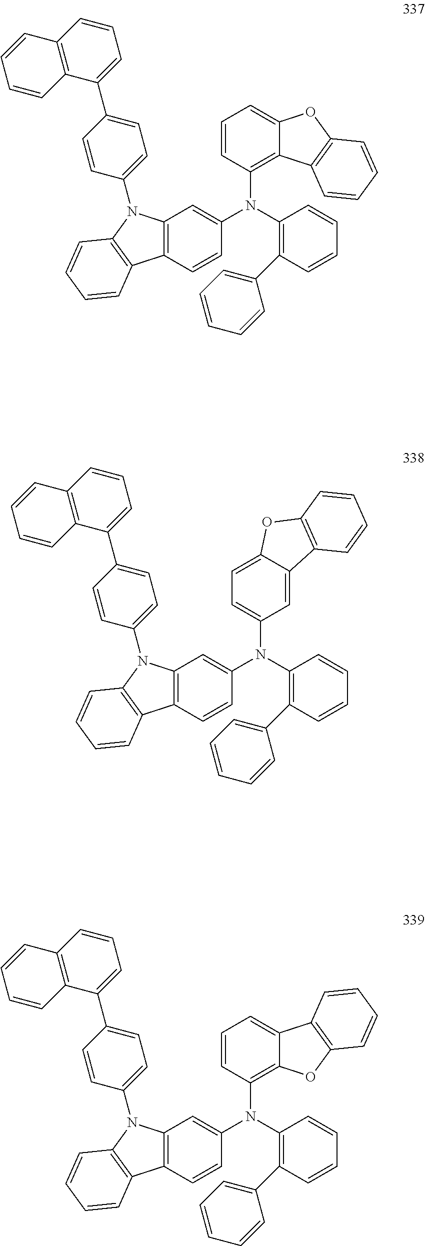 [SMILES: c1ccc(-c2ccccc2N(c2ccc3c4ccccc4n(-c4ccc(-c5cccc6ccccc56)cc4)c3c2)c2cccc3oc4ccccc4c23)cc1 ; c1ccc(-c2ccccc2N(c2ccc3oc4ccccc4c3c2)c2ccc3c4ccccc4n(-c4ccc(-c5cccc6ccccc56)cc4)c3c2)cc1 ; c1ccc(-c2ccccc2N(c2ccc3c4ccccc4n(-c4ccc(-c5cccc6ccccc56)cc4)c3c2)c2cccc3c2oc2ccccc23)cc1] 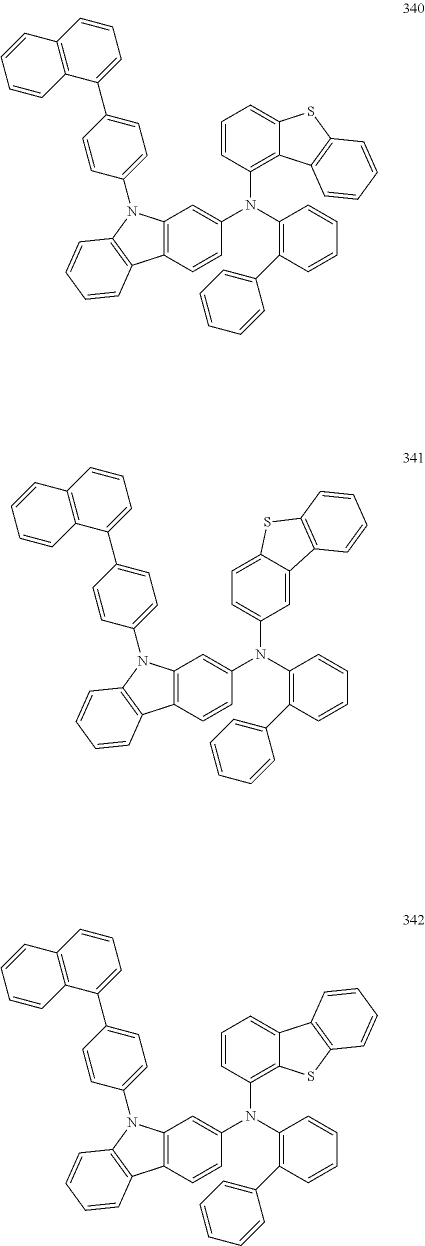 [SMILES: c1ccc(-c2ccccc2N(c2ccc3c4ccccc4n(-c4ccc(-c5cccc6ccccc56)cc4)c3c2)c2cccc3sc4ccccc4c23)cc1 ; c1ccc(-c2ccccc2N(c2ccc3sc4ccccc4c3c2)c2ccc3c4ccccc4n(-c4ccc(-c5cccc6ccccc56)cc4)c3c2)cc1 ; c1ccc(-c2ccccc2N(c2ccc3c4ccccc4n(-c4ccc(-c5cccc6ccccc56)cc4)c3c2)c2cccc3c2sc2ccccc23)cc1] 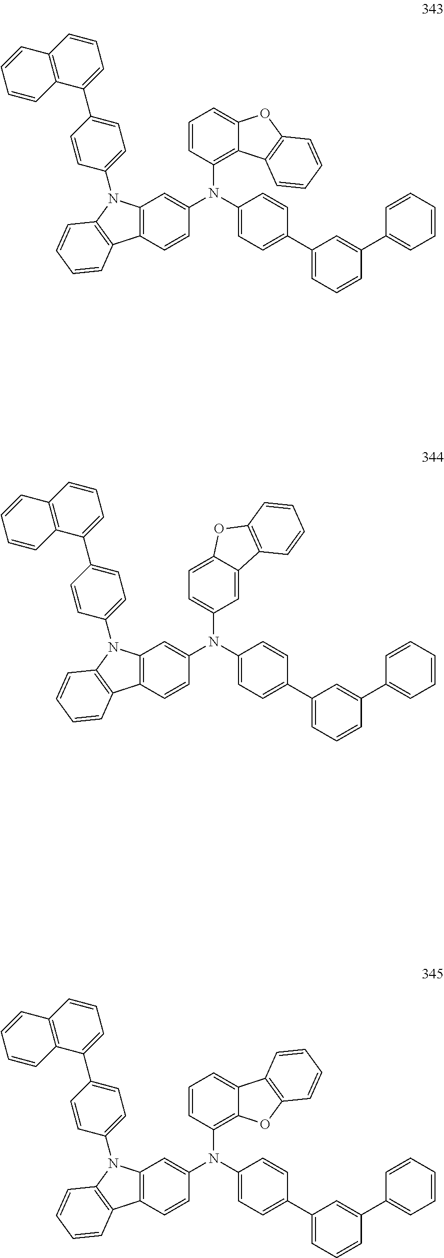 [SMILES: c1ccc(-c2cccc(-c3ccc(N(c4ccc5c6ccccc6n(-c6ccc(-c7cccc8ccccc78)cc6)c5c4)c4cccc5oc6ccccc6c45)cc3)c2)cc1 ; c1ccc(-c2cccc(-c3ccc(N(c4ccc5oc6ccccc6c5c4)c4ccc5c6ccccc6n(-c6ccc(-c7cccc8ccccc78)cc6)c5c4)cc3)c2)cc1 ; c1ccc(-c2cccc(-c3ccc(N(c4ccc5c6ccccc6n(-c6ccc(-c7cccc8ccccc78)cc6)c5c4)c4cccc5c4oc4ccccc45)cc3)c2)cc1] 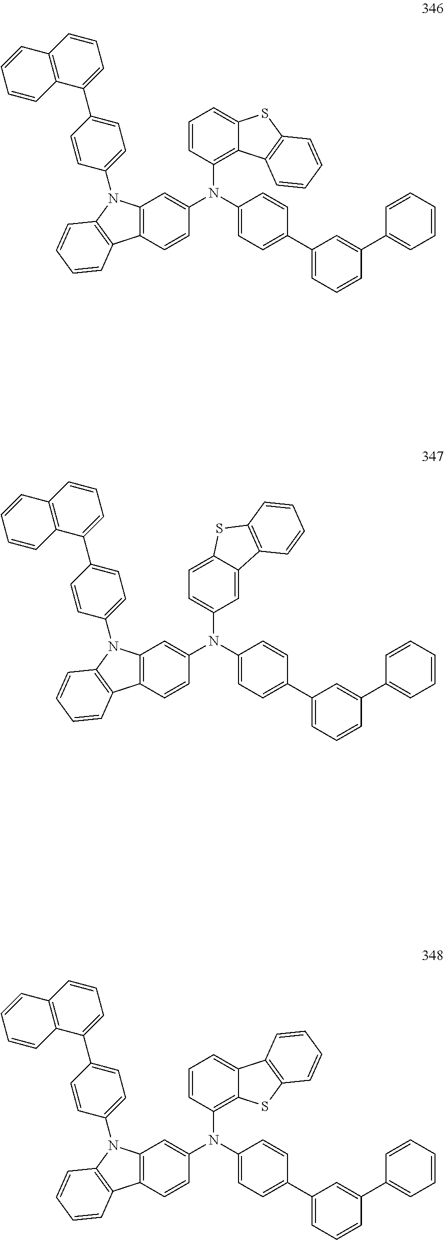 [SMILES: c1ccc(-c2cccc(-c3ccc(N(c4ccc5c6ccccc6n(-c6ccc(-c7cccc8ccccc78)cc6)c5c4)c4cccc5sc6ccccc6c45)cc3)c2)cc1 ; c1ccc(-c2cccc(-c3ccc(N(c4ccc5sc6ccccc6c5c4)c4ccc5c6ccccc6n(-c6ccc(-c7cccc8ccccc78)cc6)c5c4)cc3)c2)cc1 ; c1ccc(-c2cccc(-c3ccc(N(c4ccc5c6ccccc6n(-c6ccc(-c7cccc8ccccc78)cc6)c5c4)c4cccc5c4sc4ccccc45)cc3)c2)cc1] 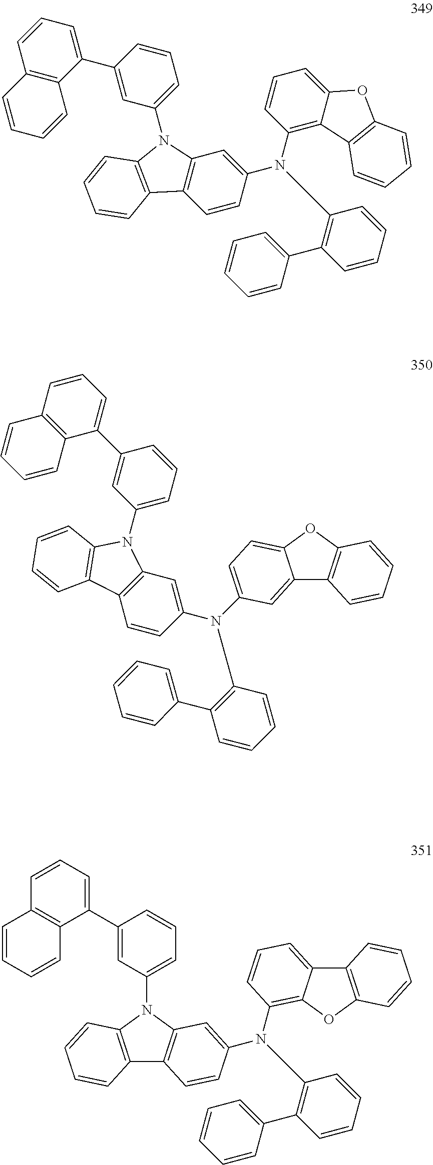 [SMILES: c1ccc(-c2ccccc2N(c2ccc3c4ccccc4n(-c4cccc(-c5cccc6ccccc56)c4)c3c2)c2cccc3oc4ccccc4c23)cc1 ; c1ccc(-c2ccccc2N(c2ccc3oc4ccccc4c3c2)c2ccc3c4ccccc4n(-c4cccc(-c5cccc6ccccc56)c4)c3c2)cc1 ; c1ccc(-c2ccccc2N(c2ccc3c4ccccc4n(-c4cccc(-c5cccc6ccccc56)c4)c3c2)c2cccc3c2oc2ccccc23)cc1] 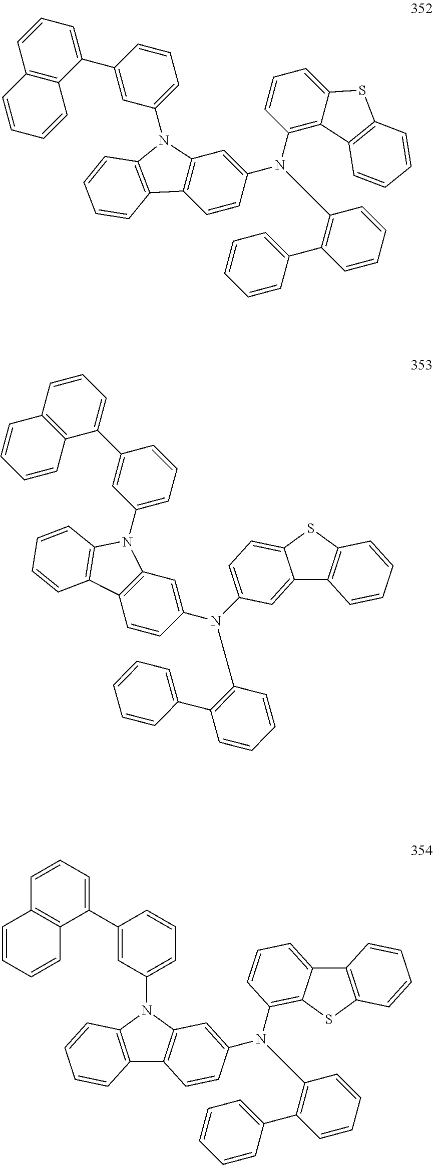 [SMILES: c1ccc(-c2ccccc2N(c2ccc3c4ccccc4n(-c4cccc(-c5cccc6ccccc56)c4)c3c2)c2cccc3sc4ccccc4c23)cc1 ; c1ccc(-c2ccccc2N(c2ccc3sc4ccccc4c3c2)c2ccc3c4ccccc4n(-c4cccc(-c5cccc6ccccc56)c4)c3c2)cc1 ; c1ccc(-c2ccccc2N(c2ccc3c4ccccc4n(-c4cccc(-c5cccc6ccccc56)c4)c3c2)c2cccc3c2sc2ccccc23)cc1] 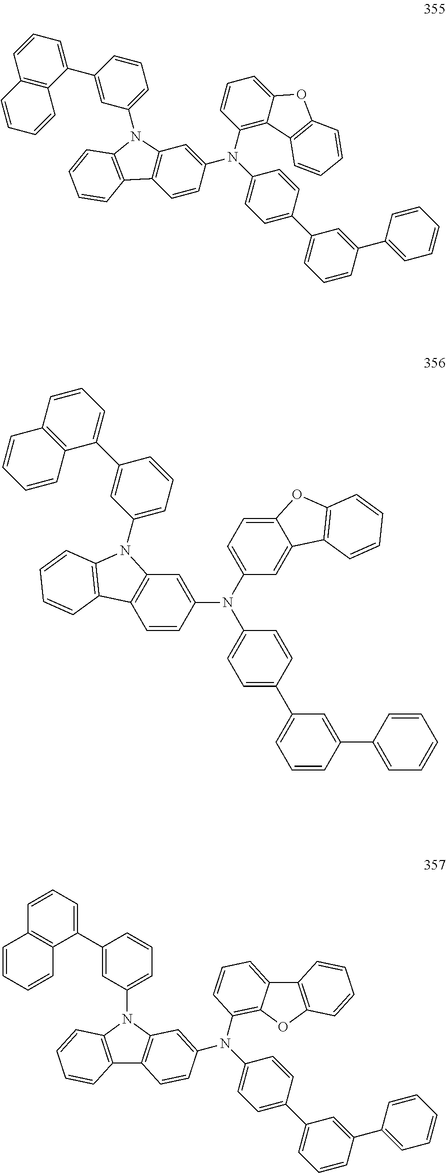 [SMILES: c1ccc(-c2cccc(-c3ccc(N(c4ccc5c6ccccc6n(-c6cccc(-c7cccc8ccccc78)c6)c5c4)c4cccc5oc6ccccc6c45)cc3)c2)cc1 ; c1ccc(-c2cccc(-c3ccc(N(c4ccc5oc6ccccc6c5c4)c4ccc5c6ccccc6n(-c6cccc(-c7cccc8ccccc78)c6)c5c4)cc3)c2)cc1 ; c1ccc(-c2cccc(-c3ccc(N(c4ccc5c6ccccc6n(-c6cccc(-c7cccc8ccccc78)c6)c5c4)c4cccc5c4oc4ccccc45)cc3)c2)cc1] 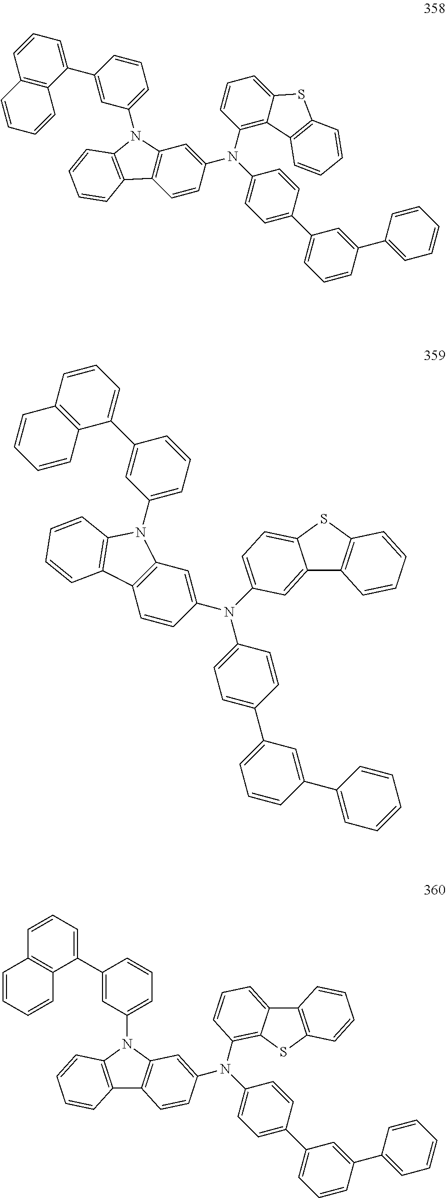 [SMILES: c1ccc(-c2cccc(-c3ccc(N(c4ccc5c6ccccc6n(-c6cccc(-c7cccc8ccccc78)c6)c5c4)c4cccc5sc6ccccc6c45)cc3)c2)cc1 ; c1ccc(-c2cccc(-c3ccc(N(c4ccc5sc6ccccc6c5c4)c4ccc5c6ccccc6n(-c6cccc(-c7cccc8ccccc78)c6)c5c4)cc3)c2)cc1 ; c1ccc(-c2cccc(-c3ccc(N(c4ccc5c6ccccc6n(-c6cccc(-c7cccc8ccccc78)c6)c5c4)c4cccc5c4sc4ccccc45)cc3)c2)cc1] 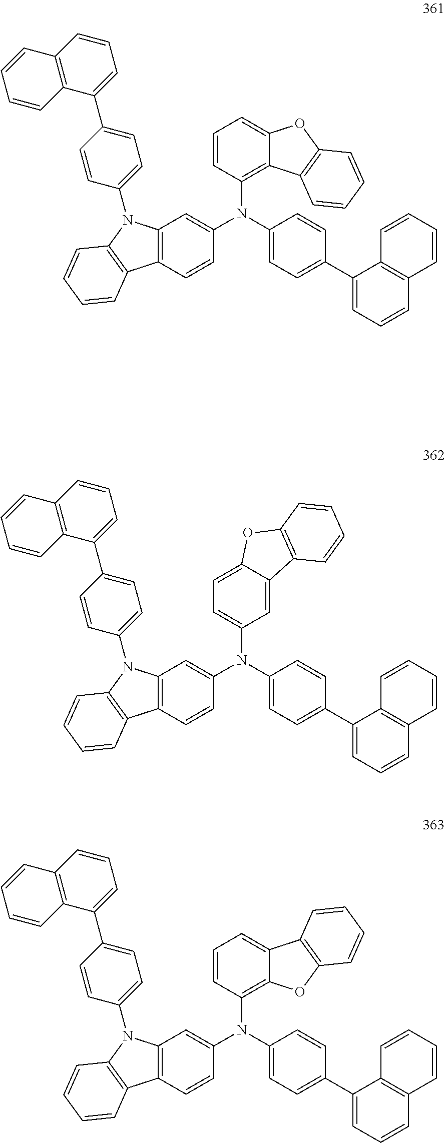 [SMILES: c1ccc2c(-c3ccc(N(c4ccc5c6ccccc6n(-c6ccc(-c7cccc8ccccc78)cc6)c5c4)c4cccc5oc6ccccc6c45)cc3)cccc2c1 ; c1ccc2c(-c3ccc(N(c4ccc5oc6ccccc6c5c4)c4ccc5c6ccccc6n(-c6ccc(-c7cccc8ccccc78)cc6)c5c4)cc3)cccc2c1 ; c1ccc2c(-c3ccc(N(c4ccc5c6ccccc6n(-c6ccc(-c7cccc8ccccc78)cc6)c5c4)c4cccc5c4oc4ccccc45)cc3)cccc2c1] 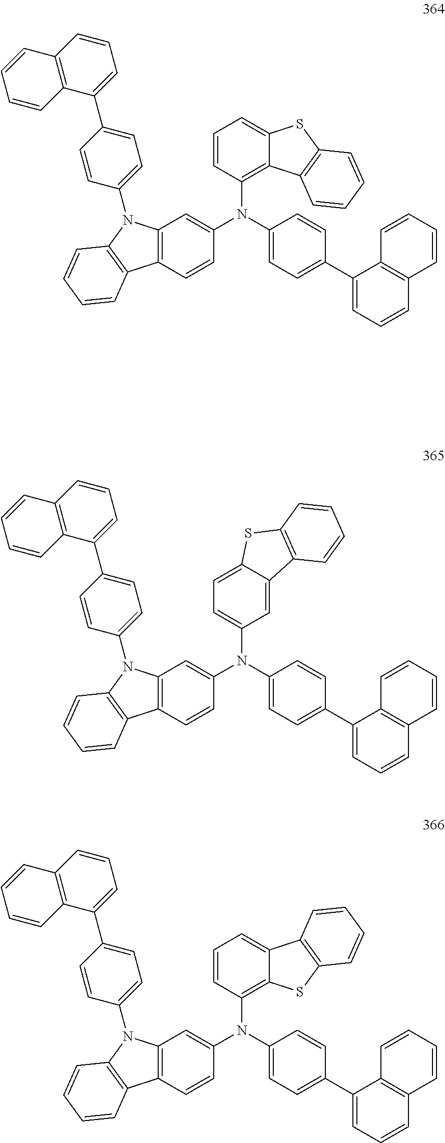 [SMILES: c1ccc2c(-c3ccc(N(c4ccc5c6ccccc6n(-c6ccc(-c7cccc8ccccc78)cc6)c5c4)c4cccc5sc6ccccc6c45)cc3)cccc2c1 ; c1ccc2c(-c3ccc(N(c4ccc5sc6ccccc6c5c4)c4ccc5c6ccccc6n(-c6ccc(-c7cccc8ccccc78)cc6)c5c4)cc3)cccc2c1 ; c1ccc2c(-c3ccc(N(c4ccc5c6ccccc6n(-c6ccc(-c7cccc8ccccc78)cc6)c5c4)c4cccc5c4sc4ccccc45)cc3)cccc2c1] 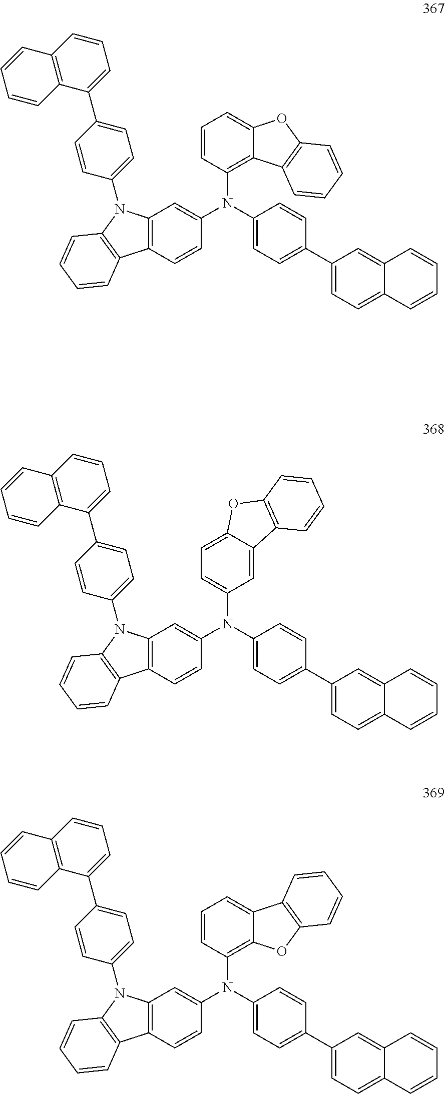 [SMILES: c1ccc2cc(-c3ccc(N(c4ccc5c6ccccc6n(-c6ccc(-c7cccc8ccccc78)cc6)c5c4)c4cccc5oc6ccccc6c45)cc3)ccc2c1 ; c1ccc2cc(-c3ccc(N(c4ccc5oc6ccccc6c5c4)c4ccc5c6ccccc6n(-c6ccc(-c7cccc8ccccc78)cc6)c5c4)cc3)ccc2c1 ; c1ccc2cc(-c3ccc(N(c4ccc5c6ccccc6n(-c6ccc(-c7cccc8ccccc78)cc6)c5c4)c4cccc5c4oc4ccccc45)cc3)ccc2c1] 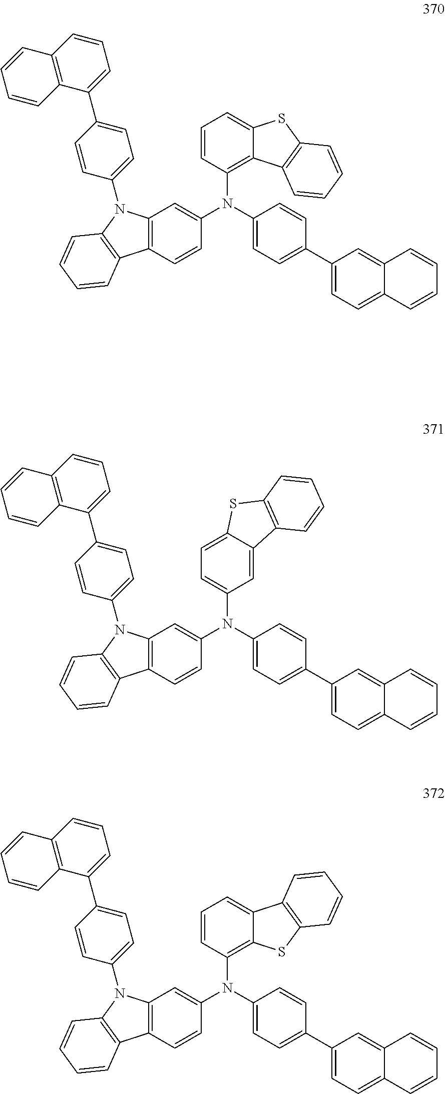 [SMILES: c1ccc2cc(-c3ccc(N(c4ccc5c6ccccc6n(-c6ccc(-c7cccc8ccccc78)cc6)c5c4)c4cccc5sc6ccccc6c45)cc3)ccc2c1 ; c1ccc2cc(-c3ccc(N(c4ccc5sc6ccccc6c5c4)c4ccc5c6ccccc6n(-c6ccc(-c7cccc8ccccc78)cc6)c5c4)cc3)ccc2c1 ; c1ccc2cc(-c3ccc(N(c4ccc5c6ccccc6n(-c6ccc(-c7cccc8ccccc78)cc6)c5c4)c4cccc5c4sc4ccccc45)cc3)ccc2c1] 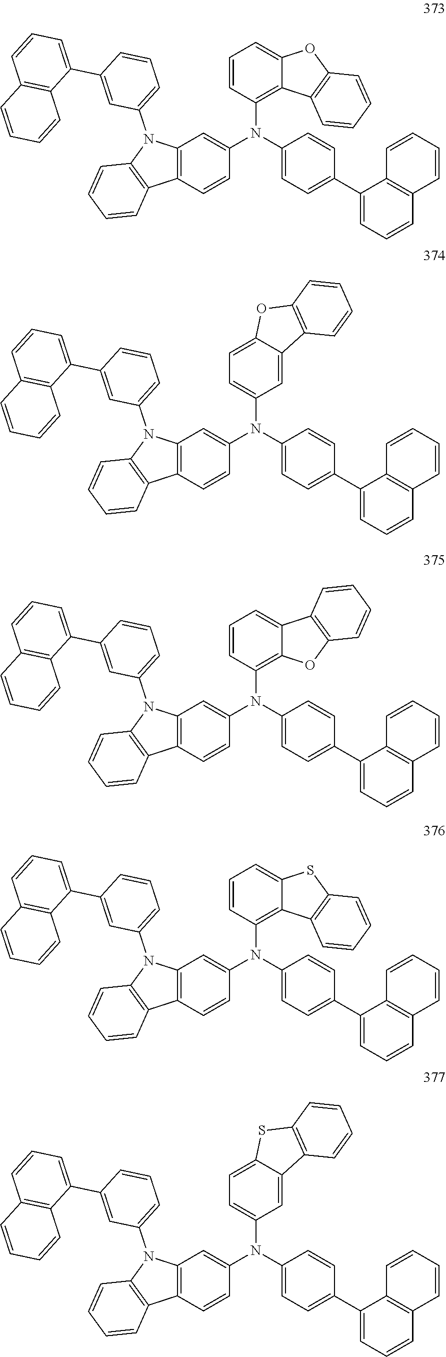 [SMILES: c1cc(-c2cccc3ccccc23)cc(-n2c3ccccc3c3ccc(N(c4ccc(-c5cccc6ccccc56)cc4)c4cccc5oc6ccccc6c45)cc32)c1 ; c1cc(-c2cccc3ccccc23)cc(-n2c3ccccc3c3ccc(N(c4ccc(-c5cccc6ccccc56)cc4)c4ccc5oc6ccccc6c5c4)cc32)c1 ; c1cc(-c2cccc3ccccc23)cc(-n2c3ccccc3c3ccc(N(c4ccc(-c5cccc6ccccc56)cc4)c4cccc5c4oc4ccccc45)cc32)c1 ; c1cc(-c2cccc3ccccc23)cc(-n2c3ccccc3c3ccc(N(c4ccc(-c5cccc6ccccc56)cc4)c4cccc5sc6ccccc6c45)cc32)c1 ; c1cc(-c2cccc3ccccc23)cc(-n2c3ccccc3c3ccc(N(c4ccc(-c5cccc6ccccc56)cc4)c4ccc5sc6ccccc6c5c4)cc32)c1] 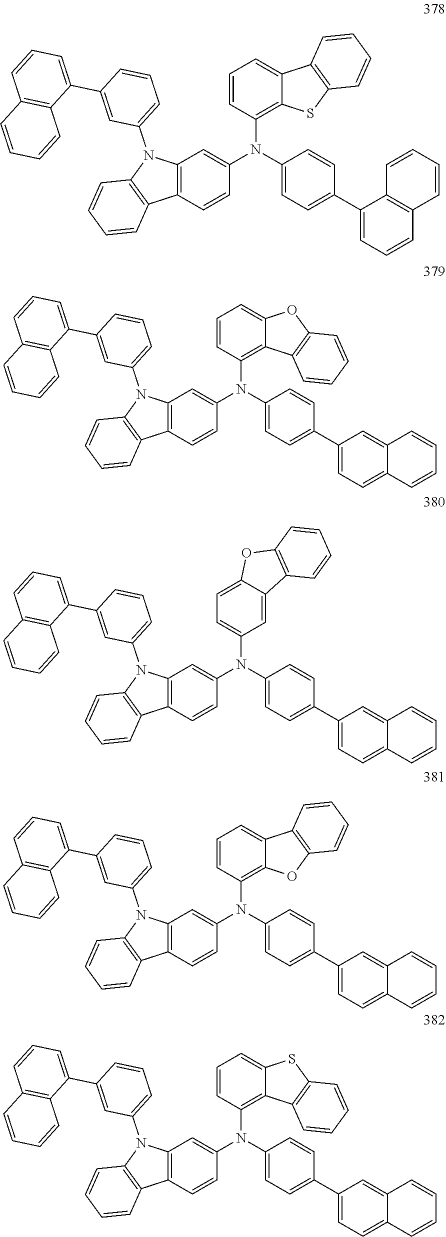 [SMILES: c1cc(-c2cccc3ccccc23)cc(-n2c3ccccc3c3ccc(N(c4ccc(-c5cccc6ccccc56)cc4)c4cccc5c4sc4ccccc45)cc32)c1 ; c1cc(-c2cccc3ccccc23)cc(-n2c3ccccc3c3ccc(N(c4ccc(-c5ccc6ccccc6c5)cc4)c4cccc5oc6ccccc6c45)cc32)c1 ; c1cc(-c2cccc3ccccc23)cc(-n2c3ccccc3c3ccc(N(c4ccc(-c5ccc6ccccc6c5)cc4)c4ccc5oc6ccccc6c5c4)cc32)c1 ; c1cc(-c2cccc3ccccc23)cc(-n2c3ccccc3c3ccc(N(c4ccc(-c5ccc6ccccc6c5)cc4)c4cccc5c4oc4ccccc45)cc32)c1 ; c1cc(-c2cccc3ccccc23)cc(-n2c3ccccc3c3ccc(N(c4ccc(-c5ccc6ccccc6c5)cc4)c4cccc5sc6ccccc6c45)cc32)c1] 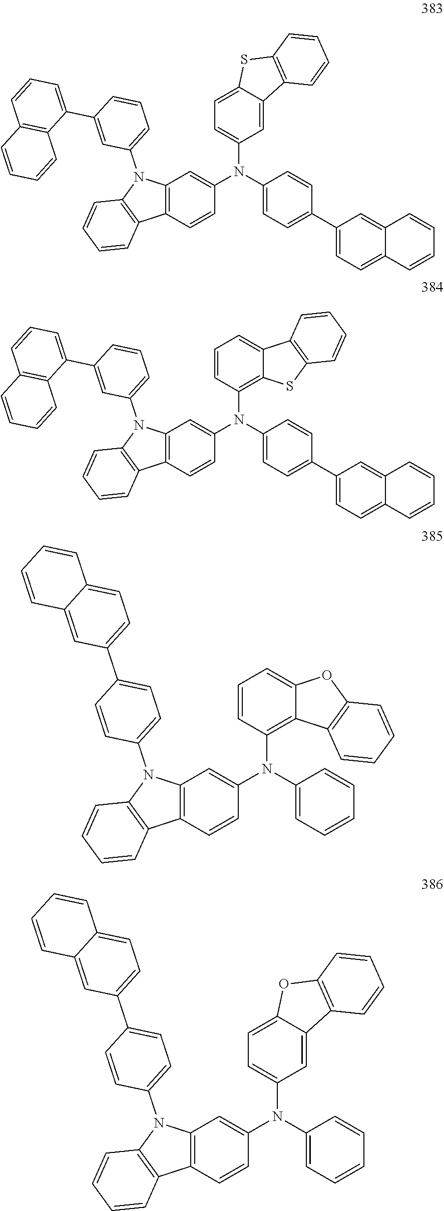 [SMILES: c1cc(-c2cccc3ccccc23)cc(-n2c3ccccc3c3ccc(N(c4ccc(-c5ccc6ccccc6c5)cc4)c4ccc5sc6ccccc6c5c4)cc32)c1 ; c1cc(-c2cccc3ccccc23)cc(-n2c3ccccc3c3ccc(N(c4ccc(-c5ccc6ccccc6c5)cc4)c4cccc5c4sc4ccccc45)cc32)c1 ; c1ccc(N(c2ccc3c4ccccc4n(-c4ccc(-c5ccc6ccccc6c5)cc4)c3c2)c2cccc3oc4ccccc4c23)cc1 ; c1ccc(N(c2ccc3oc4ccccc4c3c2)c2ccc3c4ccccc4n(-c4ccc(-c5ccc6ccccc6c5)cc4)c3c2)cc1] 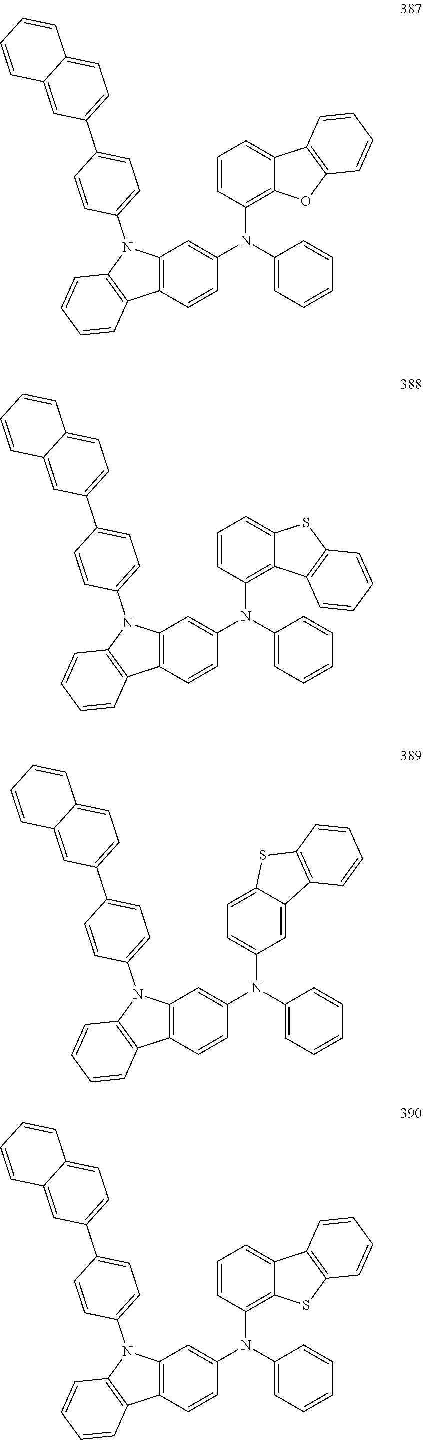 [SMILES: c1ccc(N(c2ccc3c4ccccc4n(-c4ccc(-c5ccc6ccccc6c5)cc4)c3c2)c2cccc3c2oc2ccccc23)cc1 ; c1ccc(N(c2ccc3c4ccccc4n(-c4ccc(-c5ccc6ccccc6c5)cc4)c3c2)c2cccc3sc4ccccc4c23)cc1 ; c1ccc(N(c2ccc3sc4ccccc4c3c2)c2ccc3c4ccccc4n(-c4ccc(-c5ccc6ccccc6c5)cc4)c3c2)cc1 ; c1ccc(N(c2ccc3c4ccccc4n(-c4ccc(-c5ccc6ccccc6c5)cc4)c3c2)c2cccc3c2sc2ccccc23)cc1] 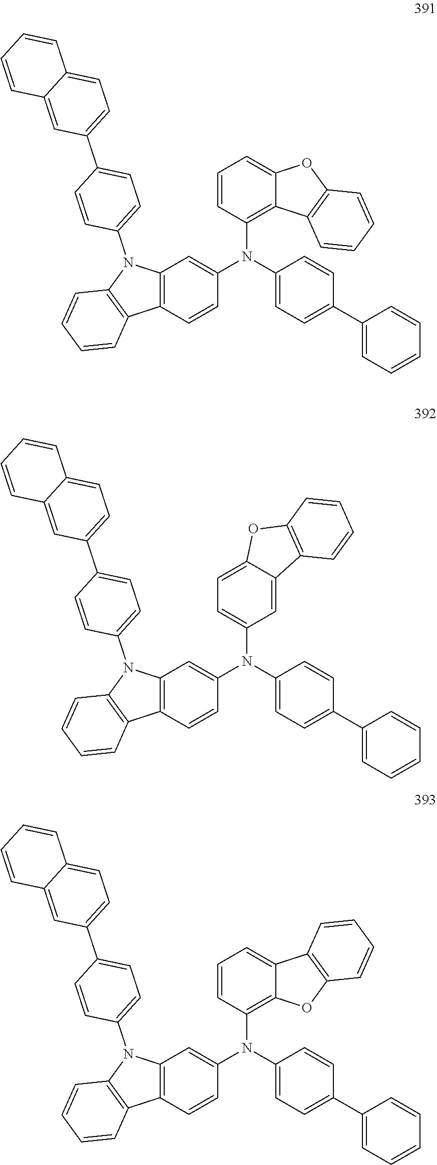 [SMILES: c1ccc(-c2ccc(N(c3ccc4c5ccccc5n(-c5ccc(-c6ccc7ccccc7c6)cc5)c4c3)c3cccc4oc5ccccc5c34)cc2)cc1 ; c1ccc(-c2ccc(N(c3ccc4oc5ccccc5c4c3)c3ccc4c5ccccc5n(-c5ccc(-c6ccc7ccccc7c6)cc5)c4c3)cc2)cc1 ; c1ccc(-c2ccc(N(c3ccc4c5ccccc5n(-c5ccc(-c6ccc7ccccc7c6)cc5)c4c3)c3cccc4c3oc3ccccc34)cc2)cc1] 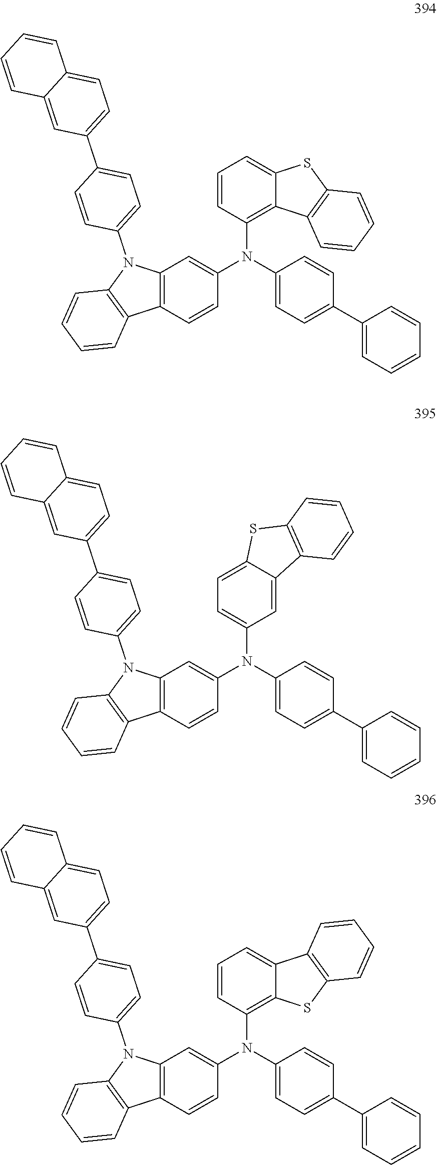 [SMILES: c1ccc(-c2ccc(N(c3ccc4c5ccccc5n(-c5ccc(-c6ccc7ccccc7c6)cc5)c4c3)c3cccc4sc5ccccc5c34)cc2)cc1 ; c1ccc(-c2ccc(N(c3ccc4sc5ccccc5c4c3)c3ccc4c5ccccc5n(-c5ccc(-c6ccc7ccccc7c6)cc5)c4c3)cc2)cc1 ; c1ccc(-c2ccc(N(c3ccc4c5ccccc5n(-c5ccc(-c6ccc7ccccc7c6)cc5)c4c3)c3cccc4c3sc3ccccc34)cc2)cc1] 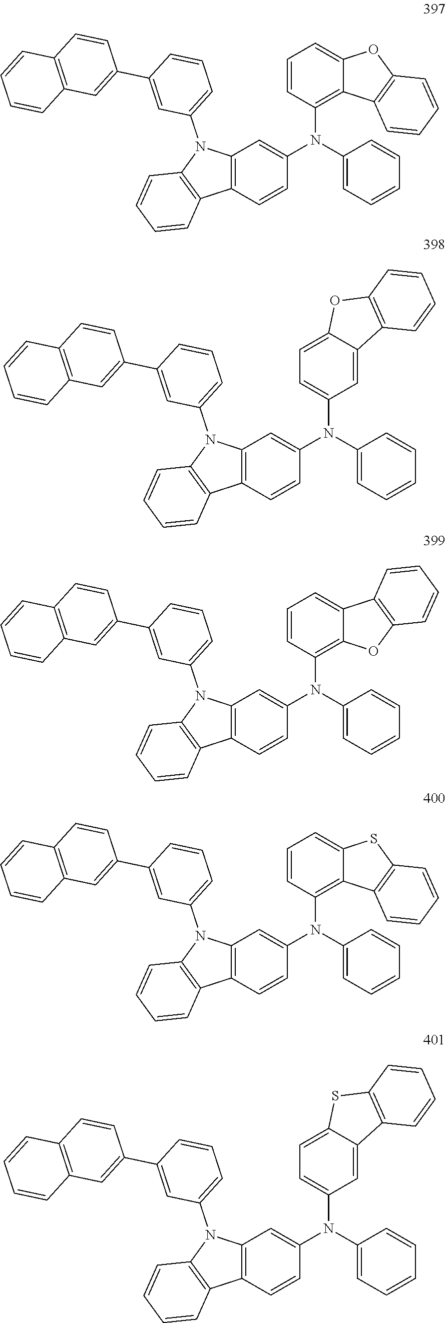 [SMILES: c1ccc(N(c2ccc3c4ccccc4n(-c4cccc(-c5ccc6ccccc6c5)c4)c3c2)c2cccc3oc4ccccc4c23)cc1 ; c1ccc(N(c2ccc3oc4ccccc4c3c2)c2ccc3c4ccccc4n(-c4cccc(-c5ccc6ccccc6c5)c4)c3c2)cc1 ; c1ccc(N(c2ccc3c4ccccc4n(-c4cccc(-c5ccc6ccccc6c5)c4)c3c2)c2cccc3c2oc2ccccc23)cc1 ; c1ccc(N(c2ccc3c4ccccc4n(-c4cccc(-c5ccc6ccccc6c5)c4)c3c2)c2cccc3sc4ccccc4c23)cc1 ; c1ccc(N(c2ccc3sc4ccccc4c3c2)c2ccc3c4ccccc4n(-c4cccc(-c5ccc6ccccc6c5)c4)c3c2)cc1] 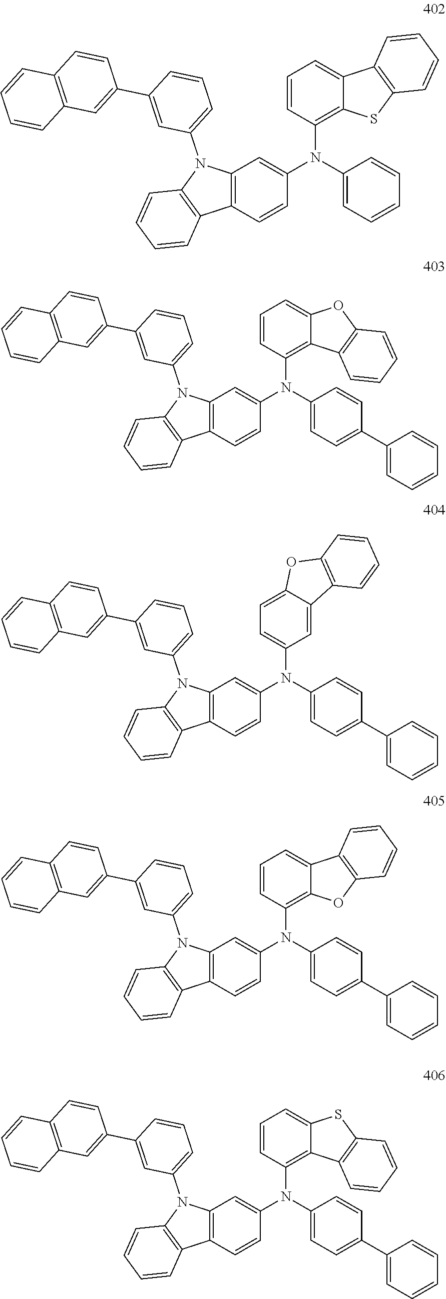 [SMILES: c1ccc(N(c2ccc3c4ccccc4n(-c4cccc(-c5ccc6ccccc6c5)c4)c3c2)c2cccc3c2sc2ccccc23)cc1 ; c1ccc(-c2ccc(N(c3ccc4c5ccccc5n(-c5cccc(-c6ccc7ccccc7c6)c5)c4c3)c3cccc4oc5ccccc5c34)cc2)cc1 ; c1ccc(-c2ccc(N(c3ccc4oc5ccccc5c4c3)c3ccc4c5ccccc5n(-c5cccc(-c6ccc7ccccc7c6)c5)c4c3)cc2)cc1 ; c1ccc(-c2ccc(N(c3ccc4c5ccccc5n(-c5cccc(-c6ccc7ccccc7c6)c5)c4c3)c3cccc4c3oc3ccccc34)cc2)cc1 ; c1ccc(-c2ccc(N(c3ccc4c5ccccc5n(-c5cccc(-c6ccc7ccccc7c6)c5)c4c3)c3cccc4sc5ccccc5c34)cc2)cc1] 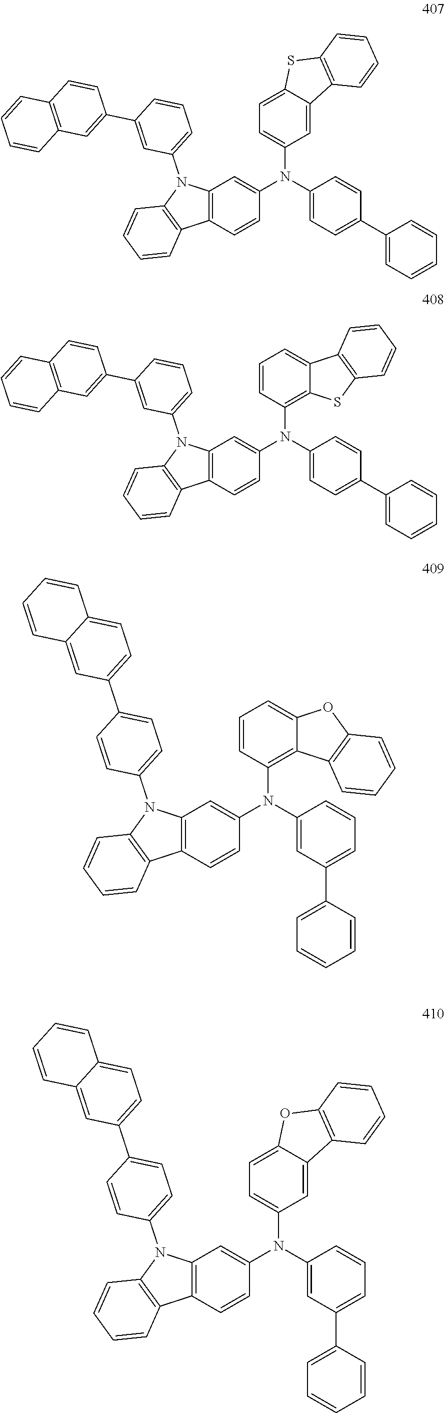 [SMILES: c1ccc(-c2ccc(N(c3ccc4sc5ccccc5c4c3)c3ccc4c5ccccc5n(-c5cccc(-c6ccc7ccccc7c6)c5)c4c3)cc2)cc1 ; c1ccc(-c2ccc(N(c3ccc4c5ccccc5n(-c5cccc(-c6ccc7ccccc7c6)c5)c4c3)c3cccc4c3sc3ccccc34)cc2)cc1 ; c1ccc(-c2cccc(N(c3ccc4c5ccccc5n(-c5ccc(-c6ccc7ccccc7c6)cc5)c4c3)c3cccc4oc5ccccc5c34)c2)cc1 ; c1ccc(-c2cccc(N(c3ccc4oc5ccccc5c4c3)c3ccc4c5ccccc5n(-c5ccc(-c6ccc7ccccc7c6)cc5)c4c3)c2)cc1] 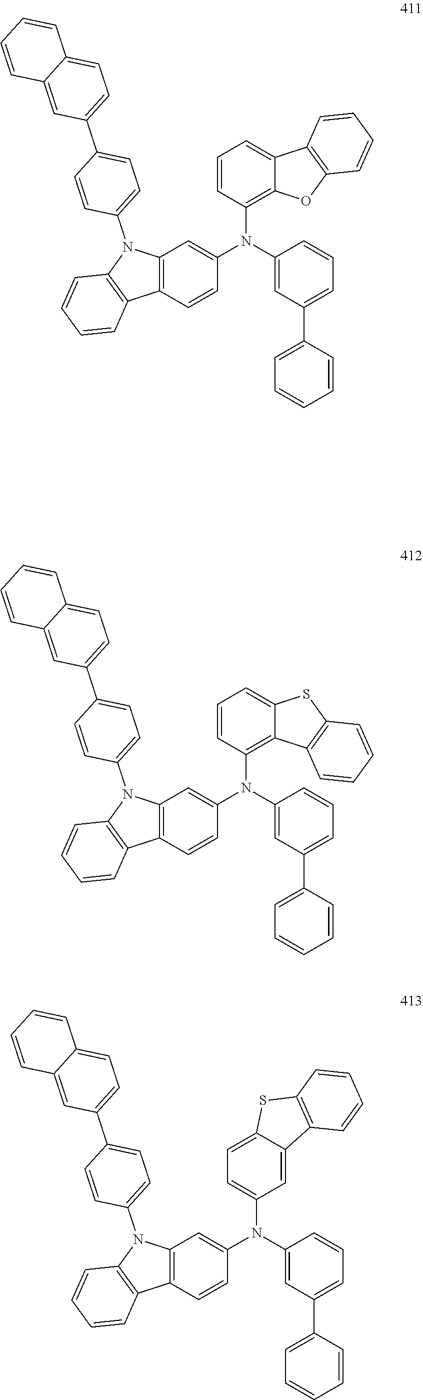 [SMILES: c1ccc(-c2cccc(N(c3ccc4c5ccccc5n(-c5ccc(-c6ccc7ccccc7c6)cc5)c4c3)c3cccc4c3oc3ccccc34)c2)cc1 ; c1ccc(-c2cccc(N(c3ccc4c5ccccc5n(-c5ccc(-c6ccc7ccccc7c6)cc5)c4c3)c3cccc4sc5ccccc5c34)c2)cc1 ; c1ccc(-c2cccc(N(c3ccc4sc5ccccc5c4c3)c3ccc4c5ccccc5n(-c5ccc(-c6ccc7ccccc7c6)cc5)c4c3)c2)cc1] 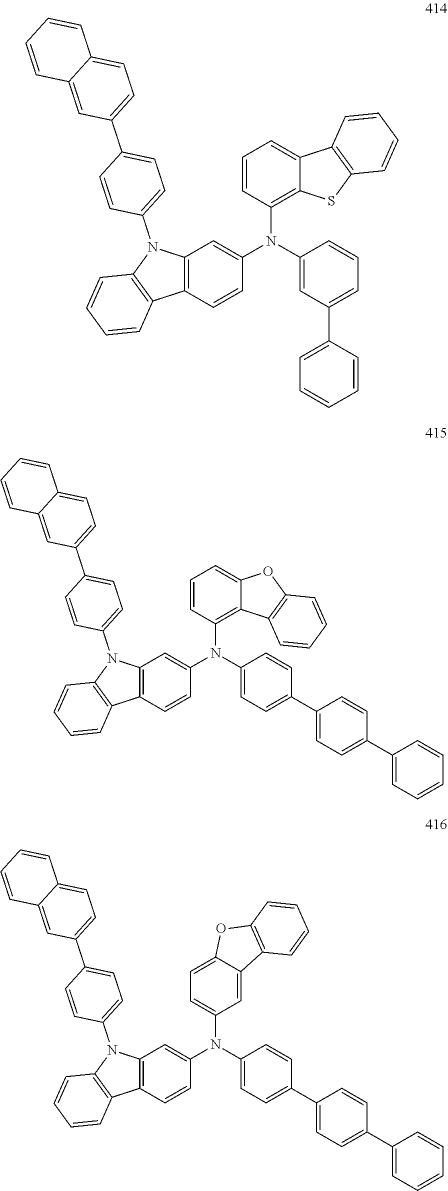 [SMILES: c1ccc(-c2cccc(N(c3ccc4c5ccccc5n(-c5ccc(-c6ccc7ccccc7c6)cc5)c4c3)c3cccc4c3sc3ccccc34)c2)cc1 ; c1ccc(-c2ccc(-c3ccc(N(c4ccc5c6ccccc6n(-c6ccc(-c7ccc8ccccc8c7)cc6)c5c4)c4cccc5oc6ccccc6c45)cc3)cc2)cc1 ; c1ccc(-c2ccc(-c3ccc(N(c4ccc5oc6ccccc6c5c4)c4ccc5c6ccccc6n(-c6ccc(-c7ccc8ccccc8c7)cc6)c5c4)cc3)cc2)cc1] 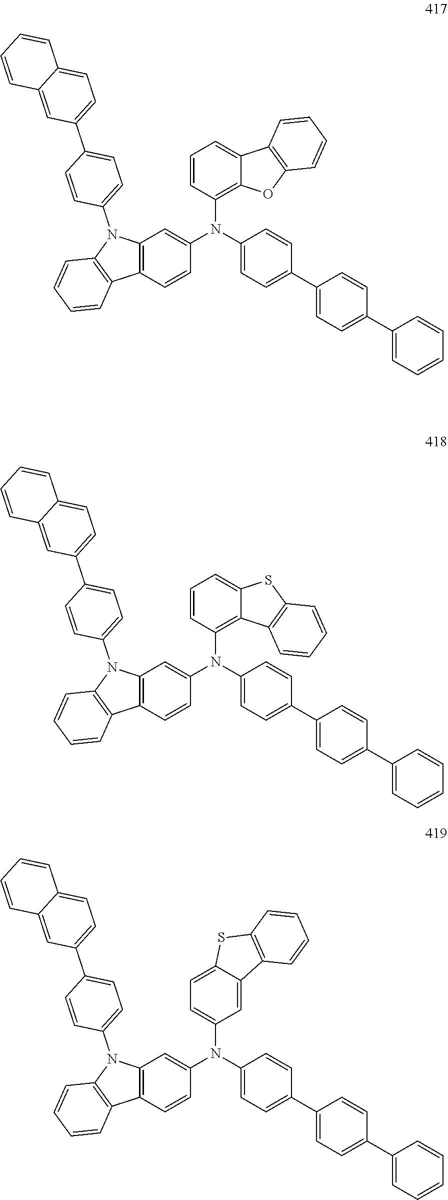 [SMILES: c1ccc(-c2ccc(-c3ccc(N(c4ccc5c6ccccc6n(-c6ccc(-c7ccc8ccccc8c7)cc6)c5c4)c4cccc5c4oc4ccccc45)cc3)cc2)cc1 ; c1ccc(-c2ccc(-c3ccc(N(c4ccc5c6ccccc6n(-c6ccc(-c7ccc8ccccc8c7)cc6)c5c4)c4cccc5sc6ccccc6c45)cc3)cc2)cc1 ; c1ccc(-c2ccc(-c3ccc(N(c4ccc5sc6ccccc6c5c4)c4ccc5c6ccccc6n(-c6ccc(-c7ccc8ccccc8c7)cc6)c5c4)cc3)cc2)cc1] 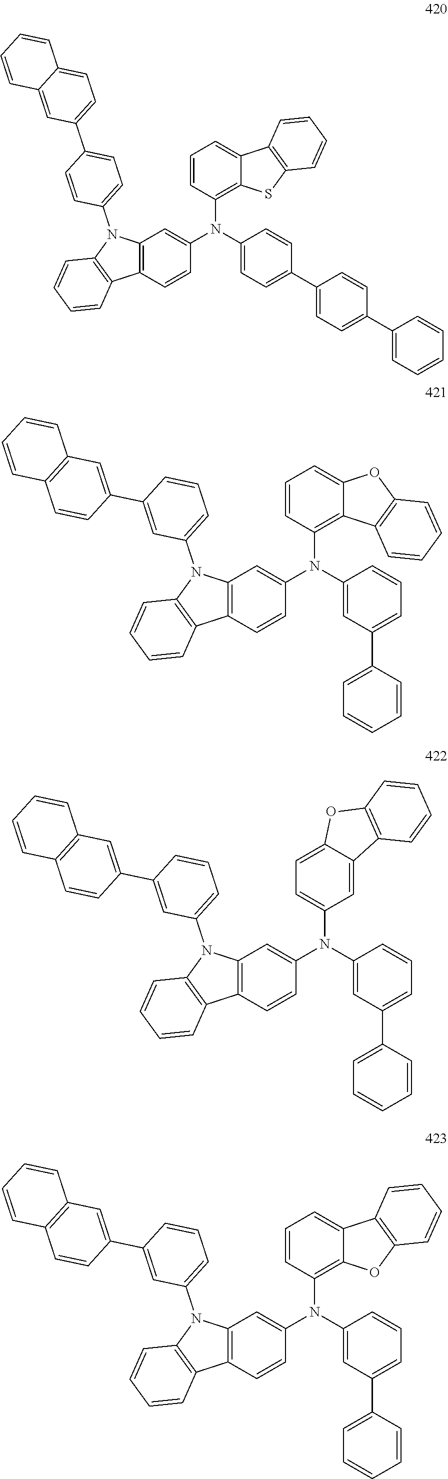 [SMILES: c1ccc(-c2ccc(-c3ccc(N(c4ccc5c6ccccc6n(-c6ccc(-c7ccc8ccccc8c7)cc6)c5c4)c4cccc5c4sc4ccccc45)cc3)cc2)cc1 ; c1ccc(-c2cccc(N(c3ccc4c5ccccc5n(-c5cccc(-c6ccc7ccccc7c6)c5)c4c3)c3cccc4oc5ccccc5c34)c2)cc1 ; c1ccc(-c2cccc(N(c3ccc4oc5ccccc5c4c3)c3ccc4c5ccccc5n(-c5cccc(-c6ccc7ccccc7c6)c5)c4c3)c2)cc1 ; c1ccc(-c2cccc(N(c3ccc4c5ccccc5n(-c5cccc(-c6ccc7ccccc7c6)c5)c4c3)c3cccc4c3oc3ccccc34)c2)cc1] 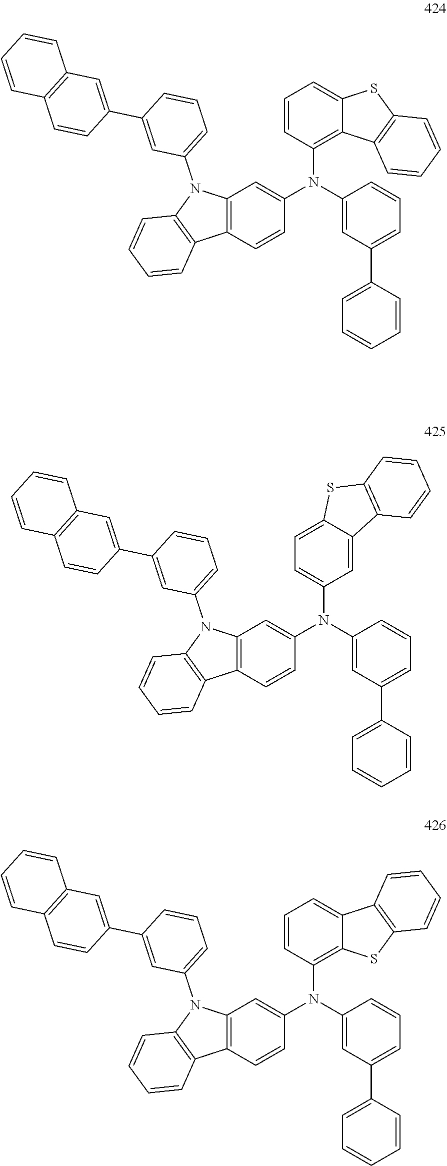 [SMILES: c1ccc(-c2cccc(N(c3ccc4c5ccccc5n(-c5cccc(-c6ccc7ccccc7c6)c5)c4c3)c3cccc4sc5ccccc5c34)c2)cc1 ; c1ccc(-c2cccc(N(c3ccc4sc5ccccc5c4c3)c3ccc4c5ccccc5n(-c5cccc(-c6ccc7ccccc7c6)c5)c4c3)c2)cc1 ; c1ccc(-c2cccc(N(c3ccc4c5ccccc5n(-c5cccc(-c6ccc7ccccc7c6)c5)c4c3)c3cccc4c3sc3ccccc34)c2)cc1] 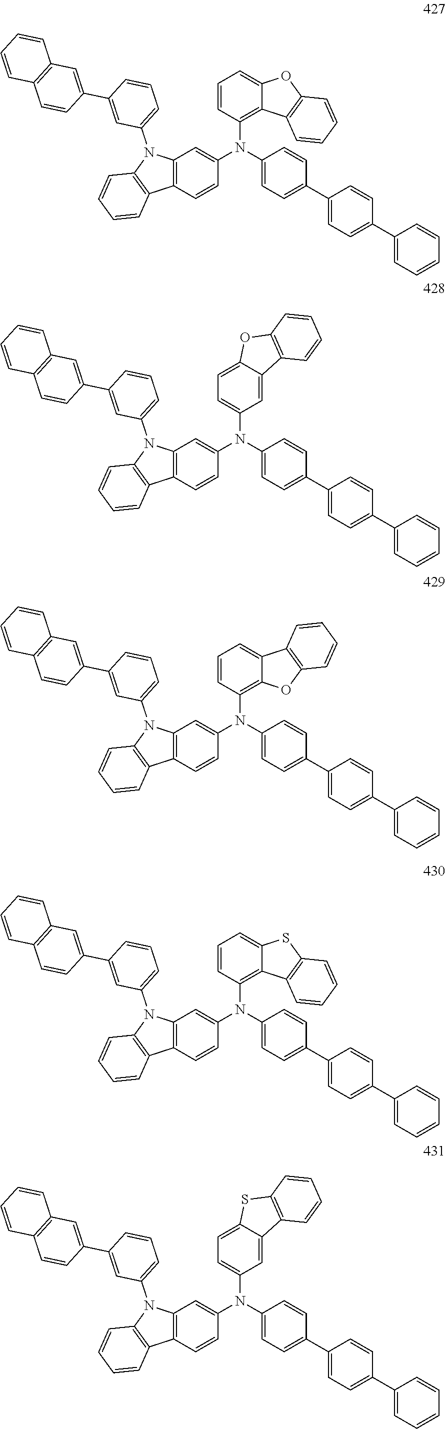 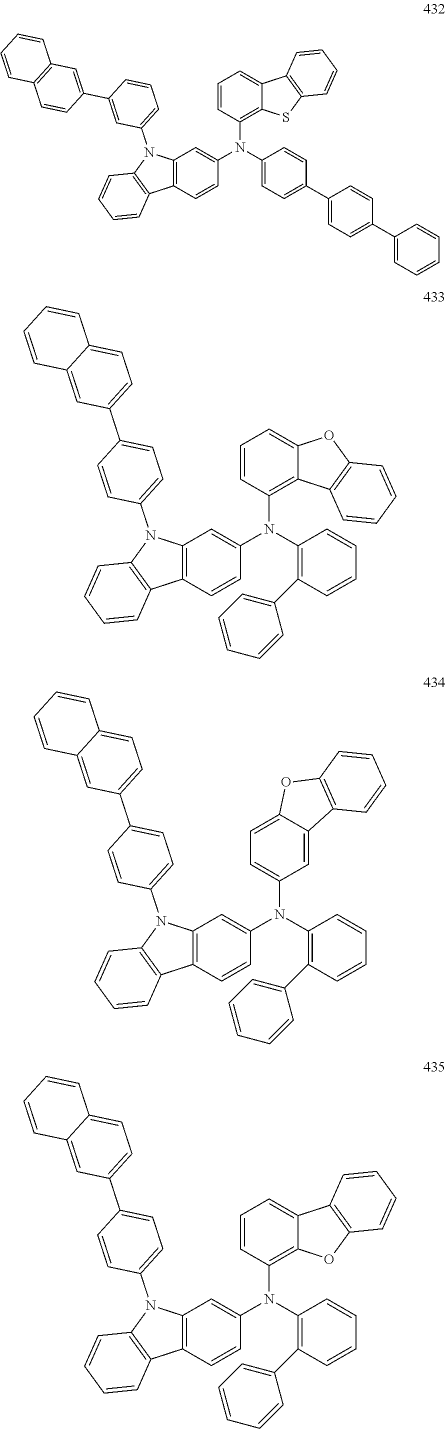 [SMILES: c1ccc(-c2ccc(-c3ccc(N(c4ccc5c6ccccc6n(-c6cccc(-c7ccc8ccccc8c7)c6)c5c4)c4cccc5c4sc4ccccc45)cc3)cc2)cc1 ; c1ccc(-c2ccccc2N(c2ccc3c4ccccc4n(-c4ccc(-c5ccc6ccccc6c5)cc4)c3c2)c2cccc3oc4ccccc4c23)cc1 ; c1ccc(-c2ccccc2N(c2ccc3oc4ccccc4c3c2)c2ccc3c4ccccc4n(-c4ccc(-c5ccc6ccccc6c5)cc4)c3c2)cc1 ; c1ccc(-c2ccccc2N(c2ccc3c4ccccc4n(-c4ccc(-c5ccc6ccccc6c5)cc4)c3c2)c2cccc3c2oc2ccccc23)cc1] 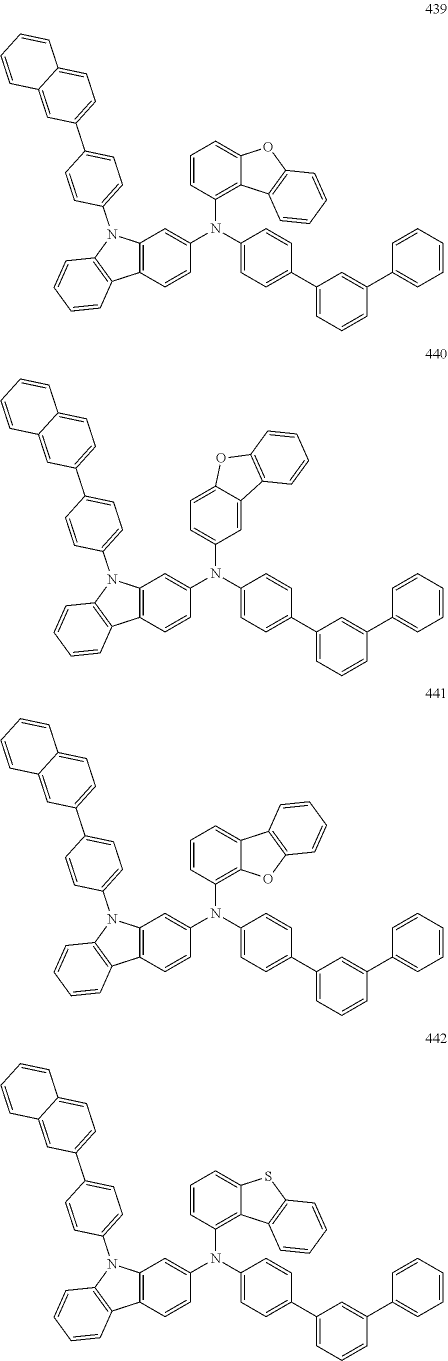 [SMILES: c1ccc(-c2cccc(-c3ccc(N(c4ccc5c6ccccc6n(-c6ccc(-c7ccc8ccccc8c7)cc6)c5c4)c4cccc5oc6ccccc6c45)cc3)c2)cc1 ; c1ccc(-c2cccc(-c3ccc(N(c4ccc5oc6ccccc6c5c4)c4ccc5c6ccccc6n(-c6ccc(-c7ccc8ccccc8c7)cc6)c5c4)cc3)c2)cc1 ; c1ccc(-c2cccc(-c3ccc(N(c4ccc5c6ccccc6n(-c6ccc(-c7ccc8ccccc8c7)cc6)c5c4)c4cccc5c4oc4ccccc45)cc3)c2)cc1 ; c1ccc(-c2cccc(-c3ccc(N(c4ccc5c6ccccc6n(-c6ccc(-c7ccc8ccccc8c7)cc6)c5c4)c4cccc5sc6ccccc6c45)cc3)c2)cc1] 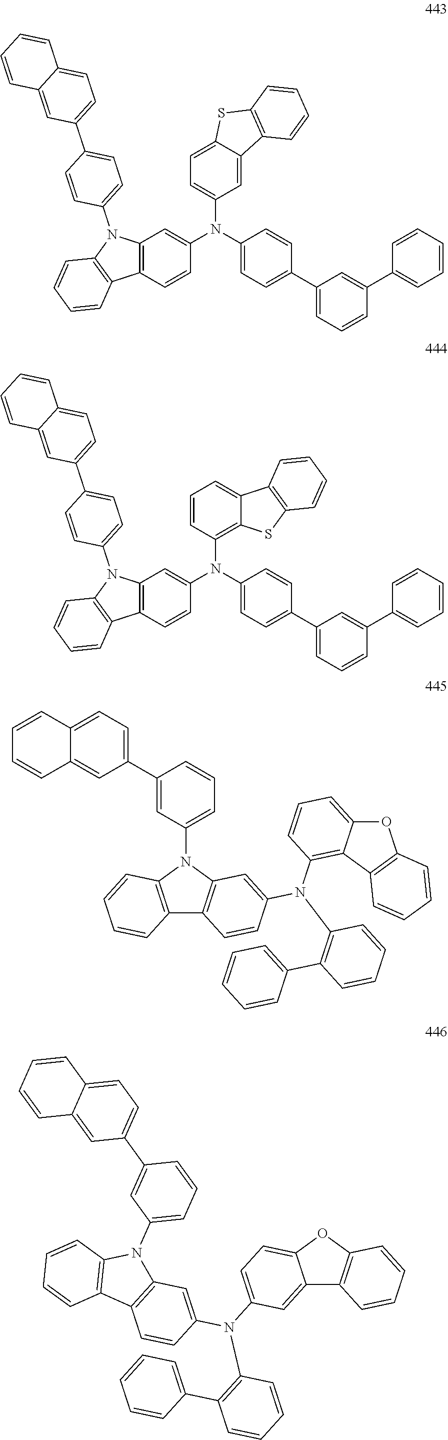 [SMILES: c1ccc(-c2cccc(-c3ccc(N(c4ccc5sc6ccccc6c5c4)c4ccc5c6ccccc6n(-c6ccc(-c7ccc8ccccc8c7)cc6)c5c4)cc3)c2)cc1 ; c1ccc(-c2cccc(-c3ccc(N(c4ccc5c6ccccc6n(-c6ccc(-c7ccc8ccccc8c7)cc6)c5c4)c4cccc5c4sc4ccccc45)cc3)c2)cc1 ; c1ccc(-c2ccccc2N(c2ccc3c4ccccc4n(-c4cccc(-c5ccc6ccccc6c5)c4)c3c2)c2cccc3oc4ccccc4c23)cc1 ; c1ccc(-c2ccccc2N(c2ccc3oc4ccccc4c3c2)c2ccc3c4ccccc4n(-c4cccc(-c5ccc6ccccc6c5)c4)c3c2)cc1] 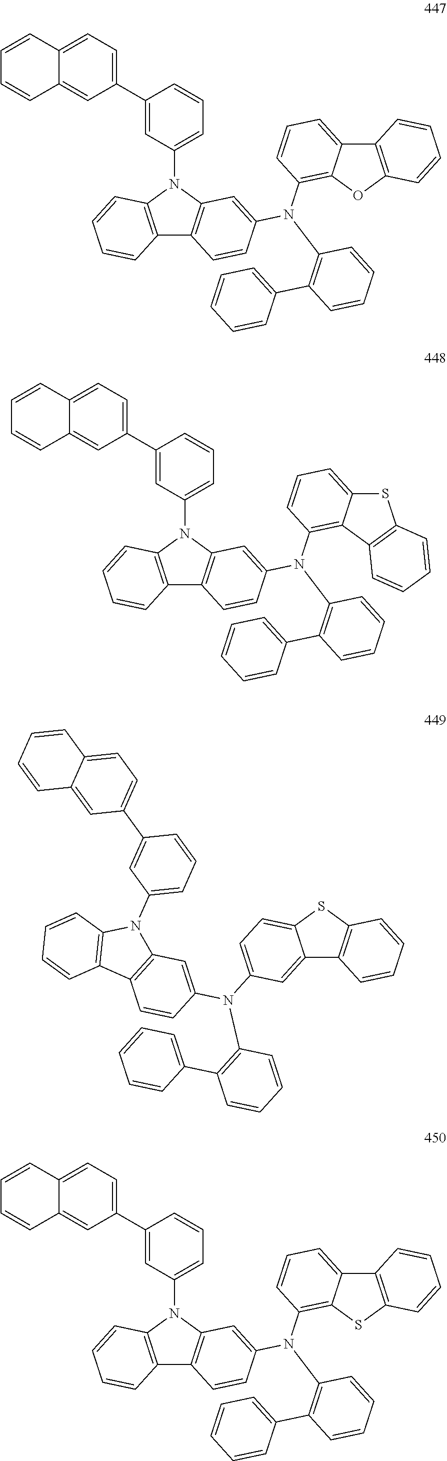 [SMILES: c1ccc(-c2ccccc2N(c2ccc3c4ccccc4n(-c4cccc(-c5ccc6ccccc6c5)c4)c3c2)c2cccc3c2oc2ccccc23)cc1 ; c1ccc(-c2ccccc2N(c2ccc3c4ccccc4n(-c4cccc(-c5ccc6ccccc6c5)c4)c3c2)c2cccc3sc4ccccc4c23)cc1 ; c1ccc(-c2ccccc2N(c2ccc3sc4ccccc4c3c2)c2ccc3c4ccccc4n(-c4cccc(-c5ccc6ccccc6c5)c4)c3c2)cc1 ; c1ccc(-c2ccccc2N(c2ccc3c4ccccc4n(-c4cccc(-c5ccc6ccccc6c5)c4)c3c2)c2cccc3c2sc2ccccc23)cc1] 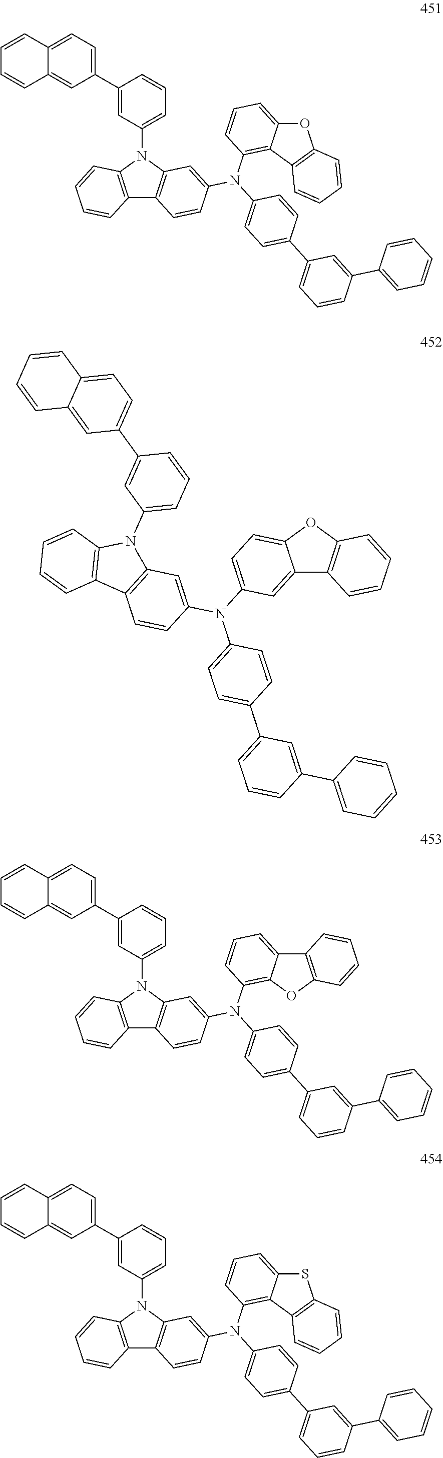 [SMILES: c1ccc(-c2cccc(-c3ccc(N(c4ccc5c6ccccc6n(-c6cccc(-c7ccc8ccccc8c7)c6)c5c4)c4cccc5oc6ccccc6c45)cc3)c2)cc1 ; c1ccc(-c2cccc(-c3ccc(N(c4ccc5oc6ccccc6c5c4)c4ccc5c6ccccc6n(-c6cccc(-c7ccc8ccccc8c7)c6)c5c4)cc3)c2)cc1 ; c1ccc(-c2cccc(-c3ccc(N(c4ccc5c6ccccc6n(-c6cccc(-c7ccc8ccccc8c7)c6)c5c4)c4cccc5c4oc4ccccc45)cc3)c2)cc1 ; c1ccc(-c2cccc(-c3ccc(N(c4ccc5c6ccccc6n(-c6cccc(-c7ccc8ccccc8c7)c6)c5c4)c4cccc5sc6ccccc6c45)cc3)c2)cc1] 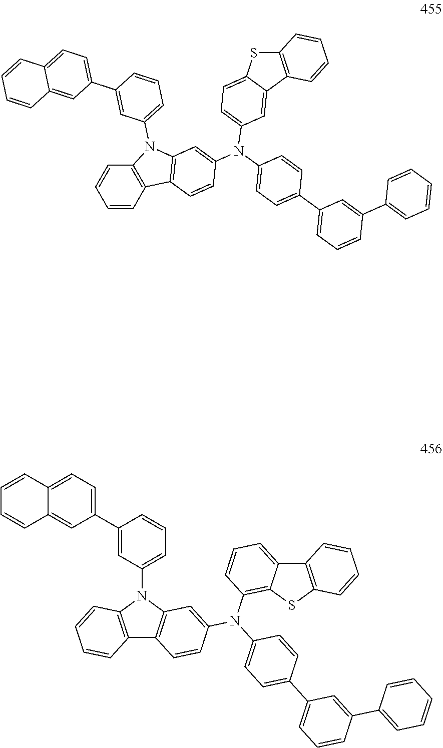 [SMILES: c1ccc(-c2cccc(-c3ccc(N(c4ccc5sc6ccccc6c5c4)c4ccc5c6ccccc6n(-c6cccc(-c7ccc8ccccc8c7)c6)c5c4)cc3)c2)cc1 ; c1ccc(-c2cccc(-c3ccc(N(c4ccc5c6ccccc6n(-c6cccc(-c7ccc8ccccc8c7)c6)c5c4)c4cccc5c4sc4ccccc45)cc3)c2)cc1] 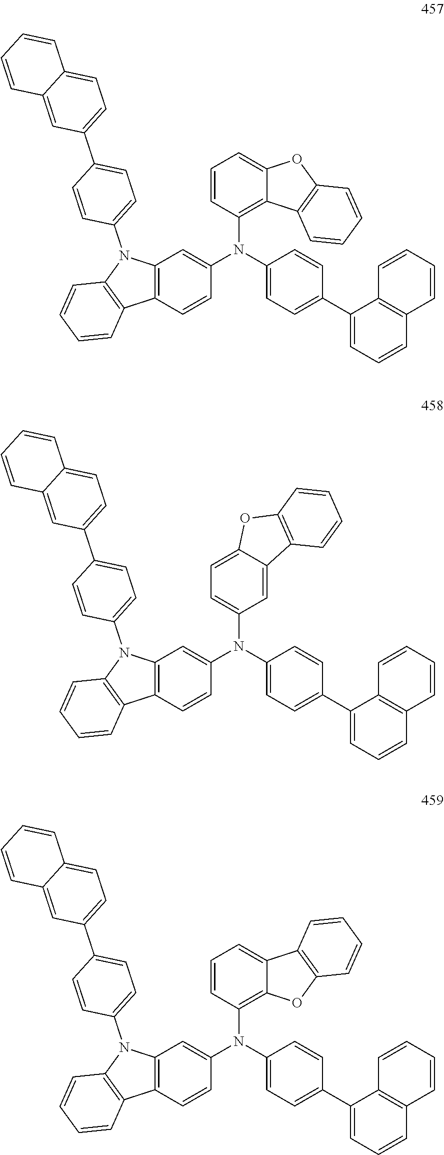 [SMILES: c1ccc2cc(-c3ccc(-n4c5ccccc5c5ccc(N(c6ccc(-c7cccc8ccccc78)cc6)c6cccc7oc8ccccc8c67)cc54)cc3)ccc2c1 ; c1ccc2cc(-c3ccc(-n4c5ccccc5c5ccc(N(c6ccc(-c7cccc8ccccc78)cc6)c6ccc7oc8ccccc8c7c6)cc54)cc3)ccc2c1 ; c1ccc2cc(-c3ccc(-n4c5ccccc5c5ccc(N(c6ccc(-c7cccc8ccccc78)cc6)c6cccc7c6oc6ccccc67)cc54)cc3)ccc2c1] 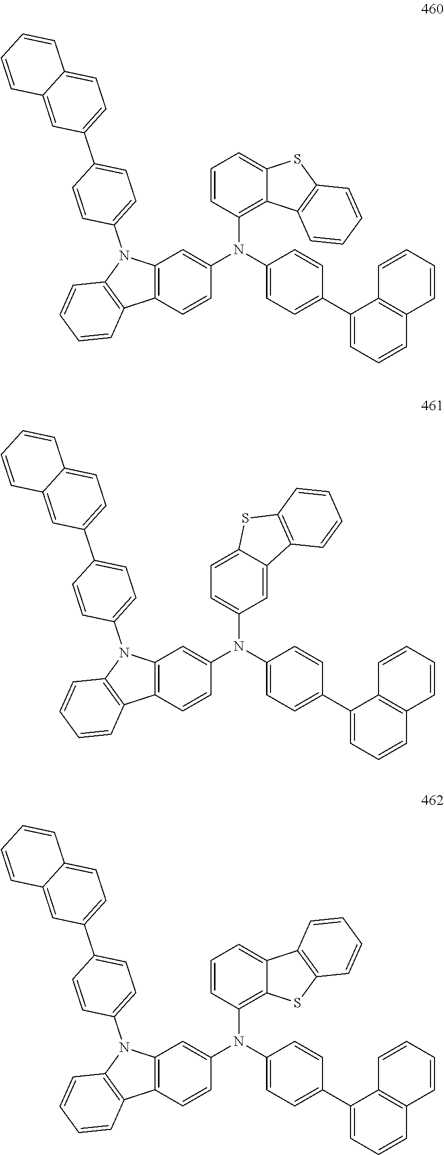 [SMILES: c1ccc2cc(-c3ccc(-n4c5ccccc5c5ccc(N(c6ccc(-c7cccc8ccccc78)cc6)c6cccc7sc8ccccc8c67)cc54)cc3)ccc2c1 ; c1ccc2cc(-c3ccc(-n4c5ccccc5c5ccc(N(c6ccc(-c7cccc8ccccc78)cc6)c6ccc7sc8ccccc8c7c6)cc54)cc3)ccc2c1 ; c1ccc2cc(-c3ccc(-n4c5ccccc5c5ccc(N(c6ccc(-c7cccc8ccccc78)cc6)c6cccc7c6sc6ccccc67)cc54)cc3)ccc2c1] 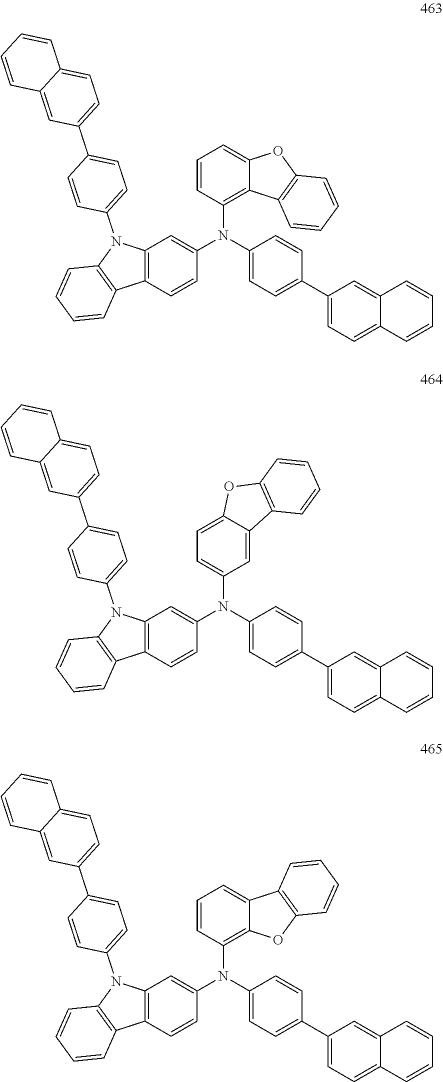 [SMILES: c1ccc2cc(-c3ccc(N(c4ccc5c6ccccc6n(-c6ccc(-c7ccc8ccccc8c7)cc6)c5c4)c4cccc5oc6ccccc6c45)cc3)ccc2c1 ; c1ccc2cc(-c3ccc(N(c4ccc5oc6ccccc6c5c4)c4ccc5c6ccccc6n(-c6ccc(-c7ccc8ccccc8c7)cc6)c5c4)cc3)ccc2c1 ; c1ccc2cc(-c3ccc(N(c4ccc5c6ccccc6n(-c6ccc(-c7ccc8ccccc8c7)cc6)c5c4)c4cccc5c4oc4ccccc45)cc3)ccc2c1] 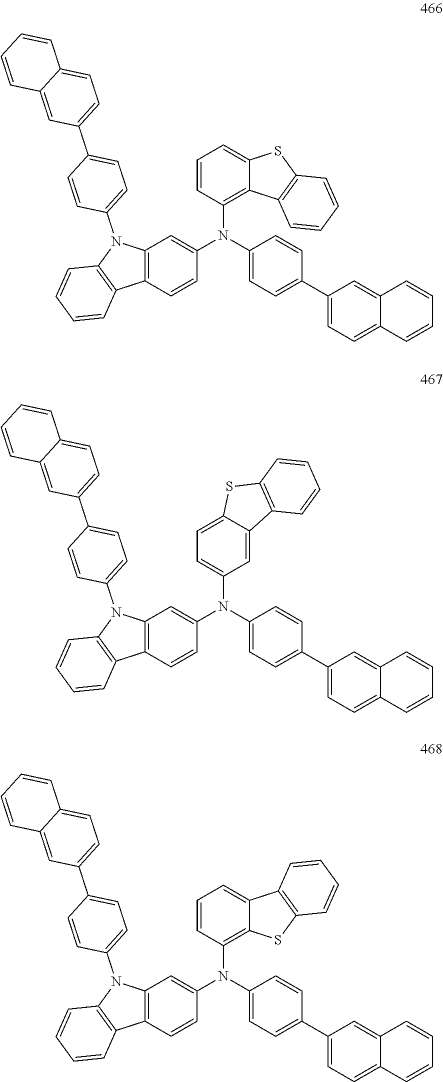 [SMILES: c1ccc2cc(-c3ccc(N(c4ccc5c6ccccc6n(-c6ccc(-c7ccc8ccccc8c7)cc6)c5c4)c4cccc5sc6ccccc6c45)cc3)ccc2c1 ; c1ccc2cc(-c3ccc(N(c4ccc5sc6ccccc6c5c4)c4ccc5c6ccccc6n(-c6ccc(-c7ccc8ccccc8c7)cc6)c5c4)cc3)ccc2c1 ; c1ccc2cc(-c3ccc(N(c4ccc5c6ccccc6n(-c6ccc(-c7ccc8ccccc8c7)cc6)c5c4)c4cccc5c4sc4ccccc45)cc3)ccc2c1] 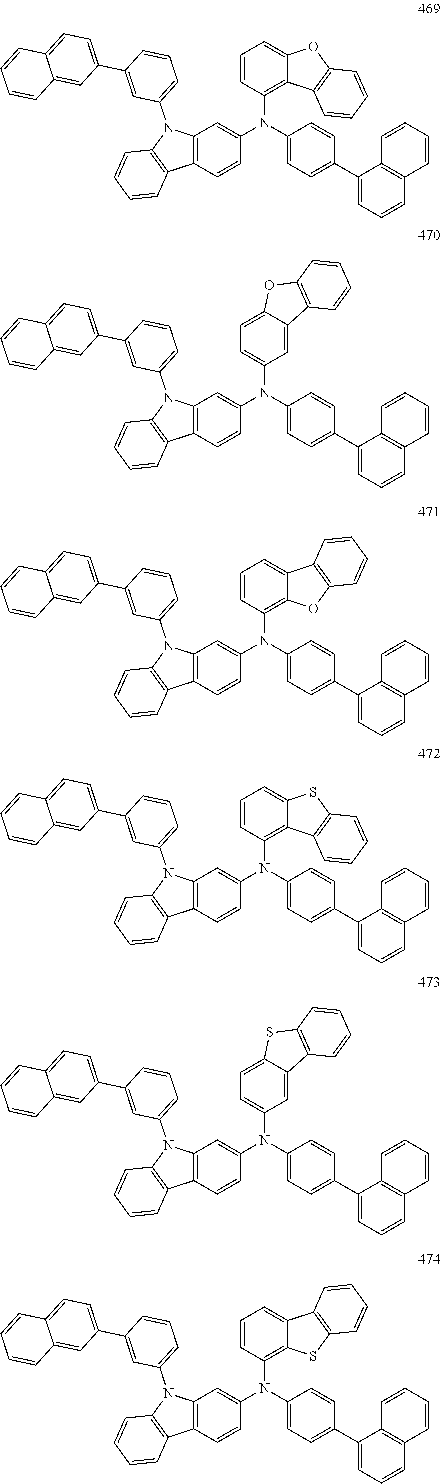 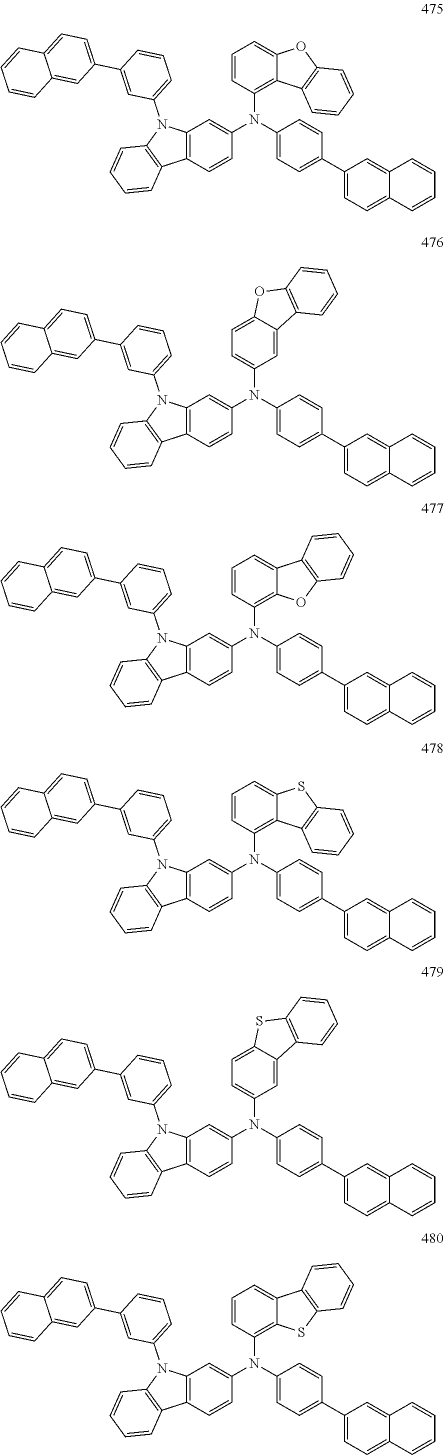 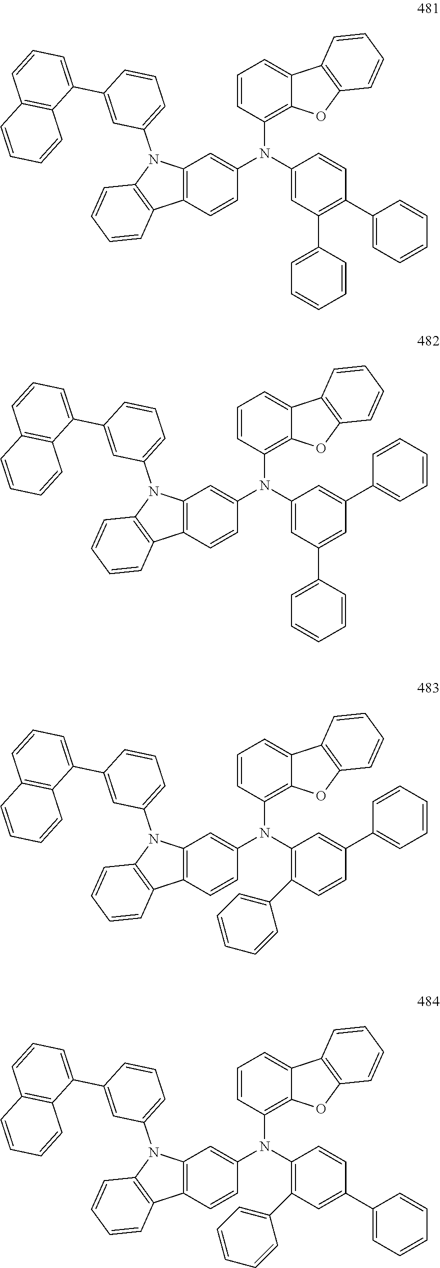 [SMILES: c1ccc(-c2ccc(N(c3ccc4c5ccccc5n(-c5cccc(-c6cccc7ccccc67)c5)c4c3)c3cccc4c3oc3ccccc34)cc2-c2ccccc2)cc1 ; c1ccc(-c2cc(-c3ccccc3)cc(N(c3ccc4c5ccccc5n(-c5cccc(-c6cccc7ccccc67)c5)c4c3)c3cccc4c3oc3ccccc34)c2)cc1 ; c1ccc(-c2ccc(-c3ccccc3)c(N(c3ccc4c5ccccc5n(-c5cccc(-c6cccc7ccccc67)c5)c4c3)c3cccc4c3oc3ccccc34)c2)cc1 ; c1ccc(-c2ccc(N(c3ccc4c5ccccc5n(-c5cccc(-c6cccc7ccccc67)c5)c4c3)c3cccc4c3oc3ccccc34)c(-c3ccccc3)c2)cc1] 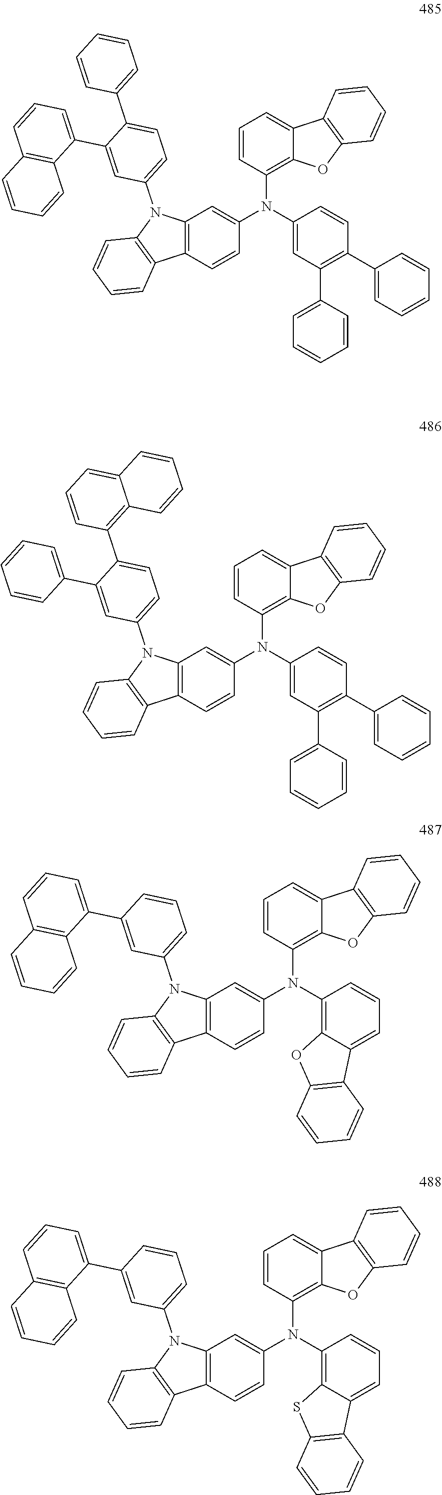 [SMILES: c1ccc(-c2ccc(N(c3ccc4c5ccccc5n(-c5ccc(-c6ccccc6)c(-c6cccc7ccccc67)c5)c4c3)c3cccc4c3oc3ccccc34)cc2-c2ccccc2)cc1 ; c1ccc(-c2ccc(N(c3ccc4c5ccccc5n(-c5ccc(-c6cccc7ccccc67)c(-c6ccccc6)c5)c4c3)c3cccc4c3oc3ccccc34)cc2-c2ccccc2)cc1 ; c1cc(-c2cccc3ccccc23)cc(-n2c3ccccc3c3ccc(N(c4cccc5c4oc4ccccc45)c4cccc5c4oc4ccccc45)cc32)c1 ; c1cc(-c2cccc3ccccc23)cc(-n2c3ccccc3c3ccc(N(c4cccc5c4oc4ccccc45)c4cccc5c4sc4ccccc45)cc32)c1] 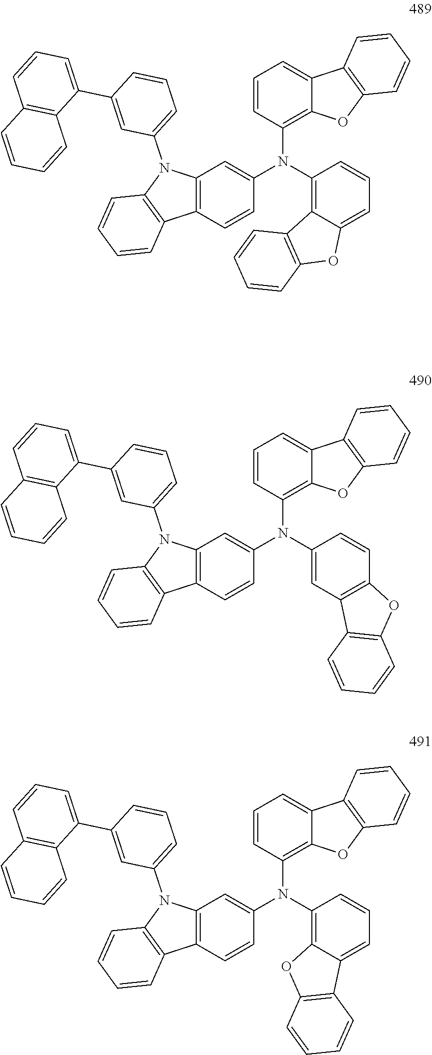 [SMILES: c1cc(-c2cccc3ccccc23)cc(-n2c3ccccc3c3ccc(N(c4cccc5c4oc4ccccc45)c4cccc5oc6ccccc6c45)cc32)c1 ; c1cc(-c2cccc3ccccc23)cc(-n2c3ccccc3c3ccc(N(c4ccc5oc6ccccc6c5c4)c4cccc5c4oc4ccccc45)cc32)c1 ; c1cc(-c2cccc3ccccc23)cc(-n2c3ccccc3c3ccc(N(c4cccc5c4oc4ccccc45)c4cccc5c4oc4ccccc45)cc32)c1] 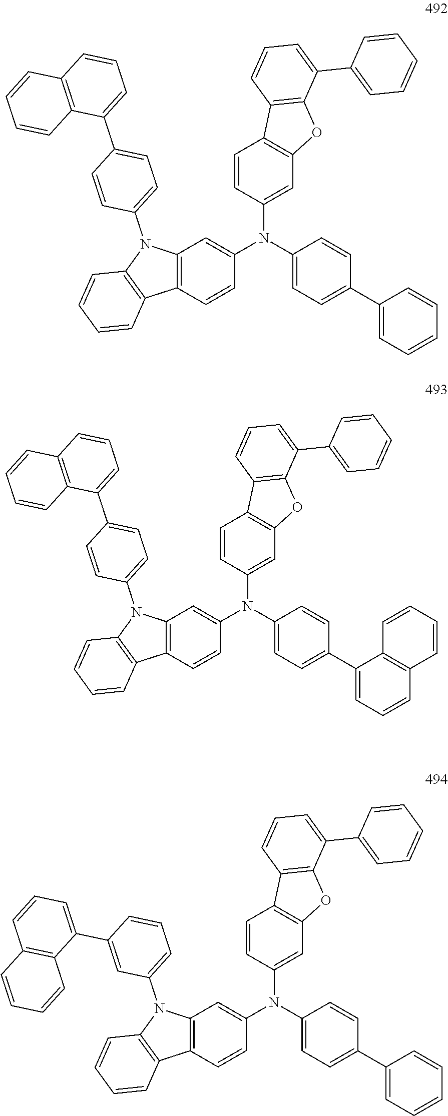 [SMILES: c1ccc(-c2ccc(N(c3ccc4c(c3)oc3c(-c5ccccc5)cccc34)c3ccc4c5ccccc5n(-c5ccc(-c6cccc7ccccc67)cc5)c4c3)cc2)cc1 ; c1ccc(-c2cccc3c2oc2cc(N(c4ccc(-c5cccc6ccccc56)cc4)c4ccc5c6ccccc6n(-c6ccc(-c7cccc8ccccc78)cc6)c5c4)ccc23)cc1 ; c1ccc(-c2ccc(N(c3ccc4c(c3)oc3c(-c5ccccc5)cccc34)c3ccc4c5ccccc5n(-c5cccc(-c6cccc7ccccc67)c5)c4c3)cc2)cc1] 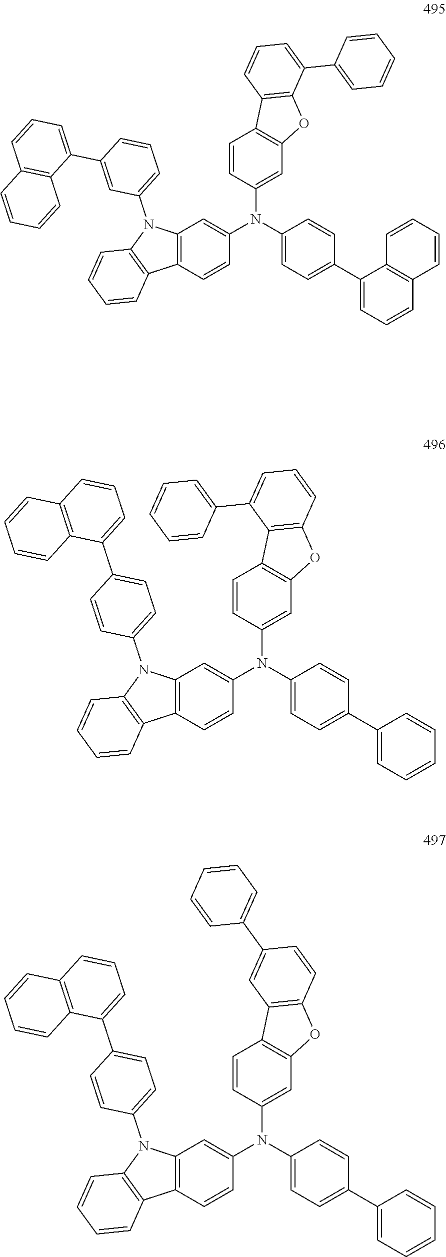 [SMILES: c1ccc(-c2cccc3c2oc2cc(N(c4ccc(-c5cccc6ccccc56)cc4)c4ccc5c6ccccc6n(-c6cccc(-c7cccc8ccccc78)c6)c5c4)ccc23)cc1 ; c1ccc(-c2ccc(N(c3ccc4c(c3)oc3cccc(-c5ccccc5)c34)c3ccc4c5ccccc5n(-c5ccc(-c6cccc7ccccc67)cc5)c4c3)cc2)cc1 ; c1ccc(-c2ccc(N(c3ccc4c(c3)oc3ccc(-c5ccccc5)cc34)c3ccc4c5ccccc5n(-c5ccc(-c6cccc7ccccc67)cc5)c4c3)cc2)cc1] 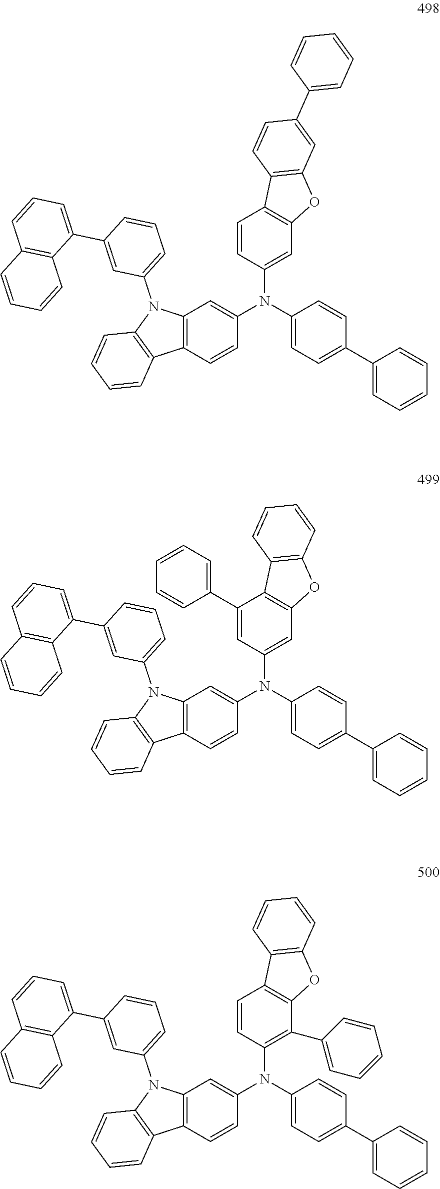 [SMILES: c1ccc(-c2ccc(N(c3ccc4c(c3)oc3cc(-c5ccccc5)ccc34)c3ccc4c5ccccc5n(-c5cccc(-c6cccc7ccccc67)c5)c4c3)cc2)cc1 ; c1ccc(-c2ccc(N(c3cc(-c4ccccc4)c4c(c3)oc3ccccc34)c3ccc4c5ccccc5n(-c5cccc(-c6cccc7ccccc67)c5)c4c3)cc2)cc1 ; c1ccc(-c2ccc(N(c3ccc4c5ccccc5n(-c5cccc(-c6cccc7ccccc67)c5)c4c3)c3ccc4c(oc5ccccc54)c3-c3ccccc3)cc2)cc1] 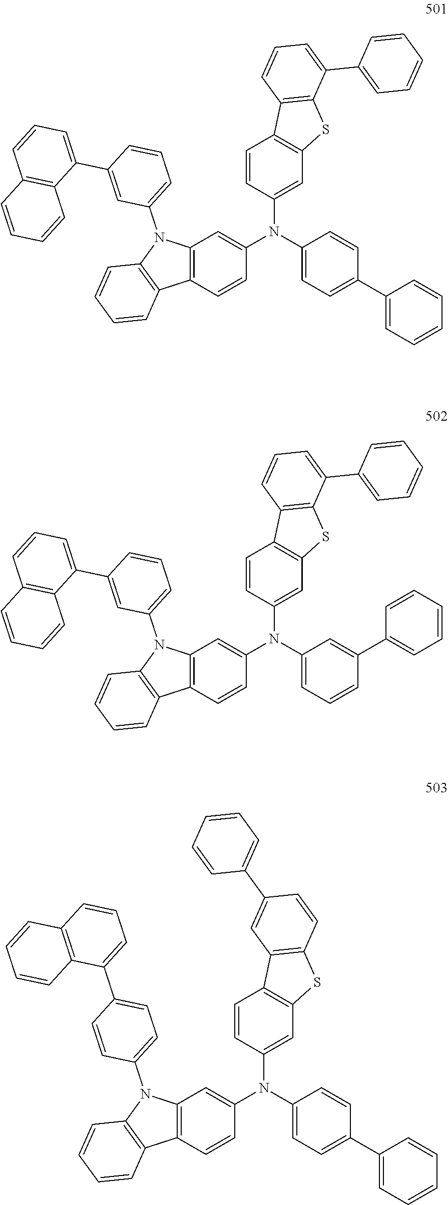 [SMILES: c1ccc(-c2ccc(N(c3ccc4c(c3)sc3c(-c5ccccc5)cccc34)c3ccc4c5ccccc5n(-c5cccc(-c6cccc7ccccc67)c5)c4c3)cc2)cc1 ; c1ccc(-c2cccc(N(c3ccc4c(c3)sc3c(-c5ccccc5)cccc34)c3ccc4c5ccccc5n(-c5cccc(-c6cccc7ccccc67)c5)c4c3)c2)cc1 ; c1ccc(-c2ccc(N(c3ccc4c(c3)sc3ccc(-c5ccccc5)cc34)c3ccc4c5ccccc5n(-c5ccc(-c6cccc7ccccc67)cc5)c4c3)cc2)cc1] 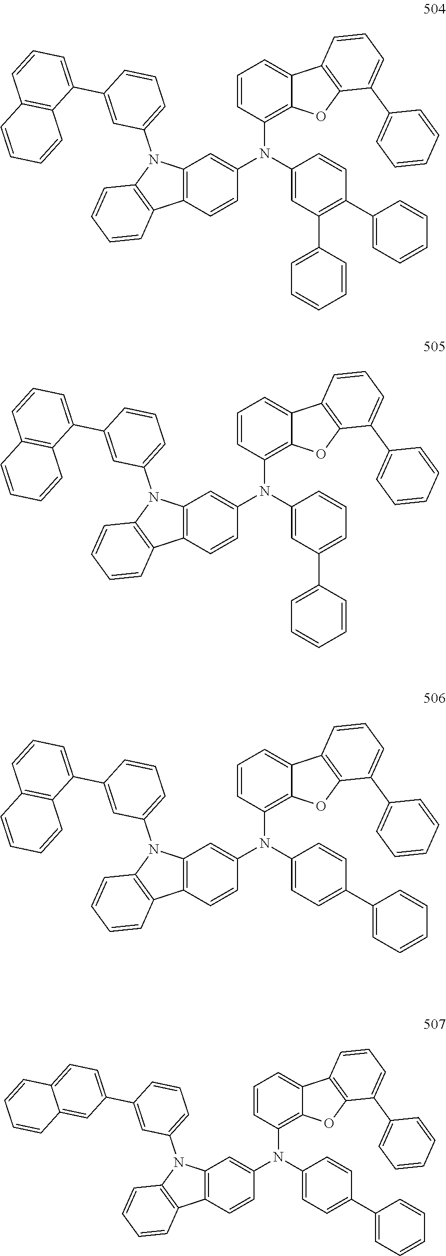 [SMILES: c1ccc(-c2ccc(N(c3ccc4c5ccccc5n(-c5cccc(-c6cccc7ccccc67)c5)c4c3)c3cccc4c3oc3c(-c5ccccc5)cccc34)cc2-c2ccccc2)cc1 ; c1ccc(-c2cccc(N(c3ccc4c5ccccc5n(-c5cccc(-c6cccc7ccccc67)c5)c4c3)c3cccc4c3oc3c(-c5ccccc5)cccc34)c2)cc1 ; c1ccc(-c2ccc(N(c3ccc4c5ccccc5n(-c5cccc(-c6cccc7ccccc67)c5)c4c3)c3cccc4c3oc3c(-c5ccccc5)cccc34)cc2)cc1 ; c1ccc(-c2ccc(N(c3ccc4c5ccccc5n(-c5cccc(-c6ccc7ccccc7c6)c5)c4c3)c3cccc4c3oc3c(-c5ccccc5)cccc34)cc2)cc1] 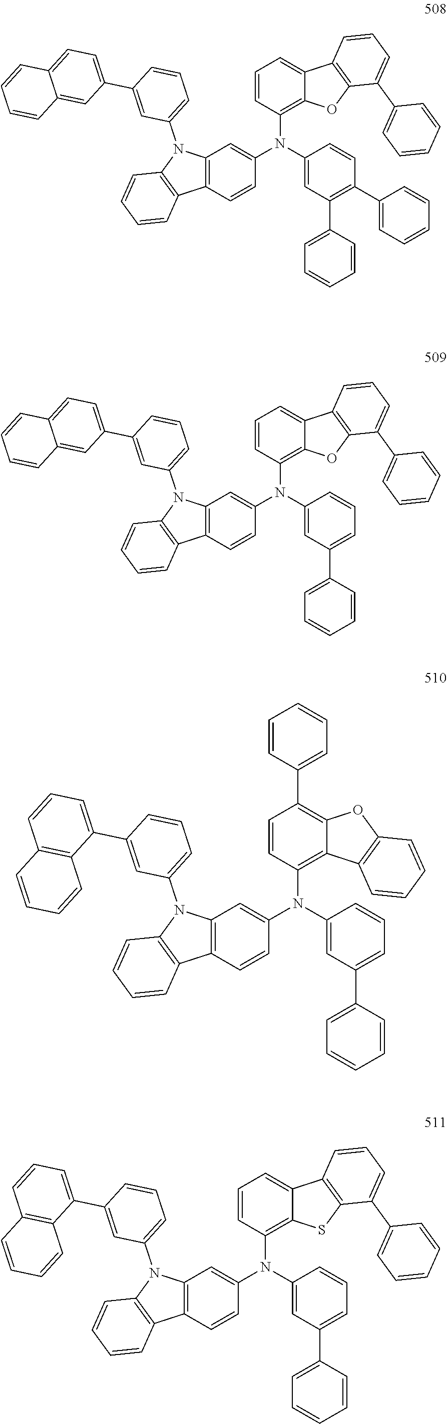 [SMILES: c1ccc(-c2ccc(N(c3ccc4c5ccccc5n(-c5cccc(-c6ccc7ccccc7c6)c5)c4c3)c3cccc4c3oc3c(-c5ccccc5)cccc34)cc2-c2ccccc2)cc1 ; c1ccc(-c2cccc(N(c3ccc4c5ccccc5n(-c5cccc(-c6ccc7ccccc7c6)c5)c4c3)c3cccc4c3oc3c(-c5ccccc5)cccc34)c2)cc1 ; c1ccc(-c2cccc(N(c3ccc4c5ccccc5n(-c5cccc(-c6cccc7ccccc67)c5)c4c3)c3ccc(-c4ccccc4)c4oc5ccccc5c34)c2)cc1 ; c1ccc(-c2cccc(N(c3ccc4c5ccccc5n(-c5cccc(-c6cccc7ccccc67)c5)c4c3)c3cccc4c3sc3c(-c5ccccc5)cccc34)c2)cc1] 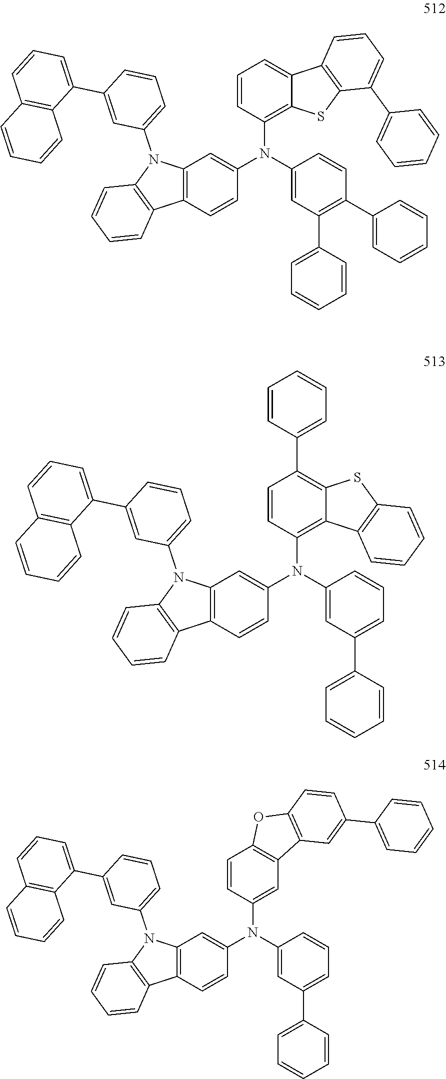 [SMILES: c1ccc(-c2ccc(N(c3ccc4c5ccccc5n(-c5cccc(-c6cccc7ccccc67)c5)c4c3)c3cccc4c3sc3c(-c5ccccc5)cccc34)cc2-c2ccccc2)cc1 ; c1ccc(-c2cccc(N(c3ccc4c5ccccc5n(-c5cccc(-c6cccc7ccccc67)c5)c4c3)c3ccc(-c4ccccc4)c4sc5ccccc5c34)c2)cc1 ; c1ccc(-c2cccc(N(c3ccc4oc5ccc(-c6ccccc6)cc5c4c3)c3ccc4c5ccccc5n(-c5cccc(-c6cccc7ccccc67)c5)c4c3)c2)cc1] 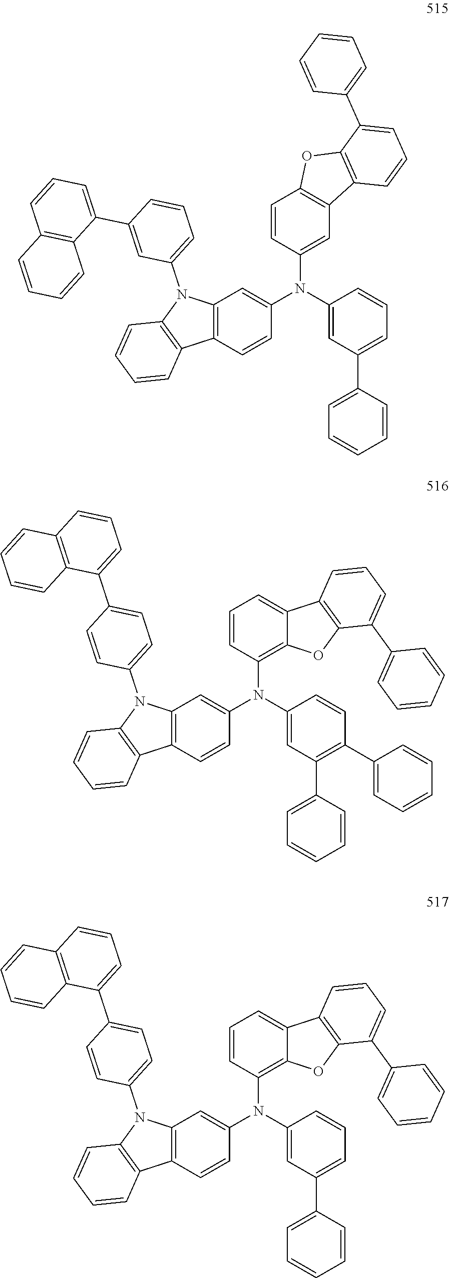 [SMILES: c1ccc(-c2cccc(N(c3ccc4oc5c(-c6ccccc6)cccc5c4c3)c3ccc4c5ccccc5n(-c5cccc(-c6cccc7ccccc67)c5)c4c3)c2)cc1 ; c1ccc(-c2ccc(N(c3ccc4c5ccccc5n(-c5ccc(-c6cccc7ccccc67)cc5)c4c3)c3cccc4c3oc3c(-c5ccccc5)cccc34)cc2-c2ccccc2)cc1 ; c1ccc(-c2cccc(N(c3ccc4c5ccccc5n(-c5ccc(-c6cccc7ccccc67)cc5)c4c3)c3cccc4c3oc3c(-c5ccccc5)cccc34)c2)cc1] 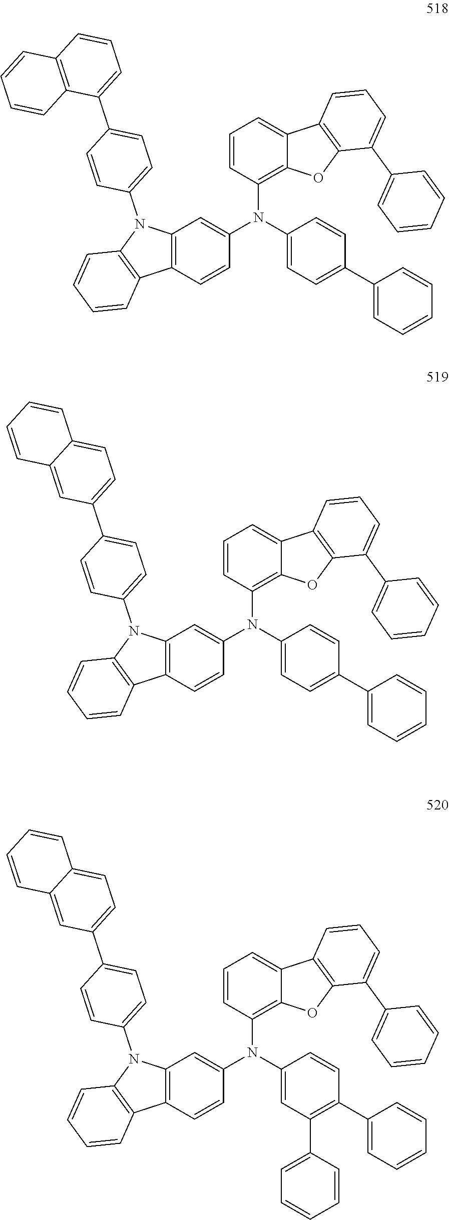 [SMILES: c1ccc(-c2ccc(N(c3ccc4c5ccccc5n(-c5ccc(-c6cccc7ccccc67)cc5)c4c3)c3cccc4c3oc3c(-c5ccccc5)cccc34)cc2)cc1 ; c1ccc(-c2ccc(N(c3ccc4c5ccccc5n(-c5ccc(-c6ccc7ccccc7c6)cc5)c4c3)c3cccc4c3oc3c(-c5ccccc5)cccc34)cc2)cc1 ; c1ccc(-c2ccc(N(c3ccc4c5ccccc5n(-c5ccc(-c6ccc7ccccc7c6)cc5)c4c3)c3cccc4c3oc3c(-c5ccccc5)cccc34)cc2-c2ccccc2)cc1] 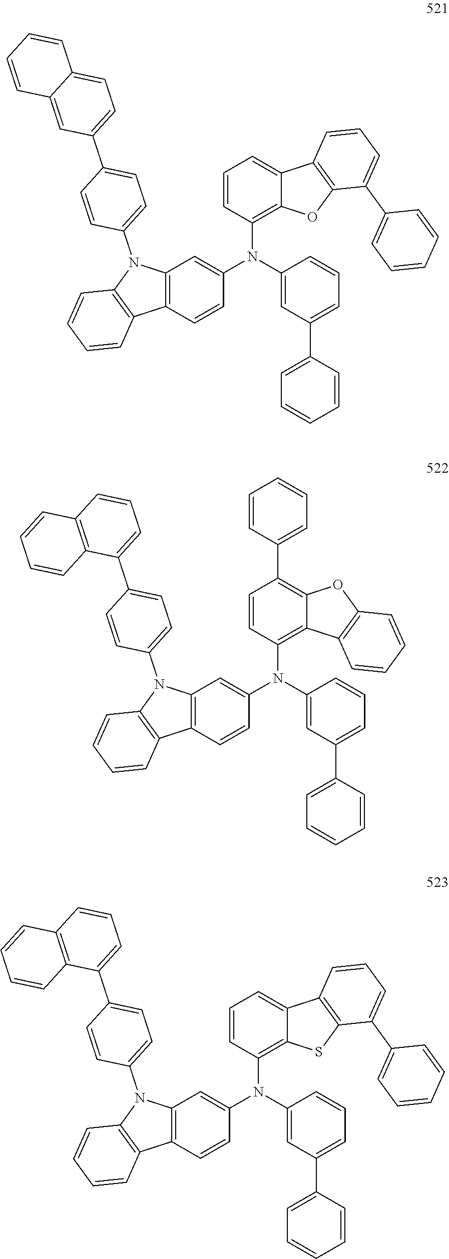 [SMILES: c1ccc(-c2cccc(N(c3ccc4c5ccccc5n(-c5ccc(-c6ccc7ccccc7c6)cc5)c4c3)c3cccc4c3oc3c(-c5ccccc5)cccc34)c2)cc1 ; c1ccc(-c2cccc(N(c3ccc4c5ccccc5n(-c5ccc(-c6cccc7ccccc67)cc5)c4c3)c3ccc(-c4ccccc4)c4oc5ccccc5c34)c2)cc1 ; c1ccc(-c2cccc(N(c3ccc4c5ccccc5n(-c5ccc(-c6cccc7ccccc67)cc5)c4c3)c3cccc4c3sc3c(-c5ccccc5)cccc34)c2)cc1] 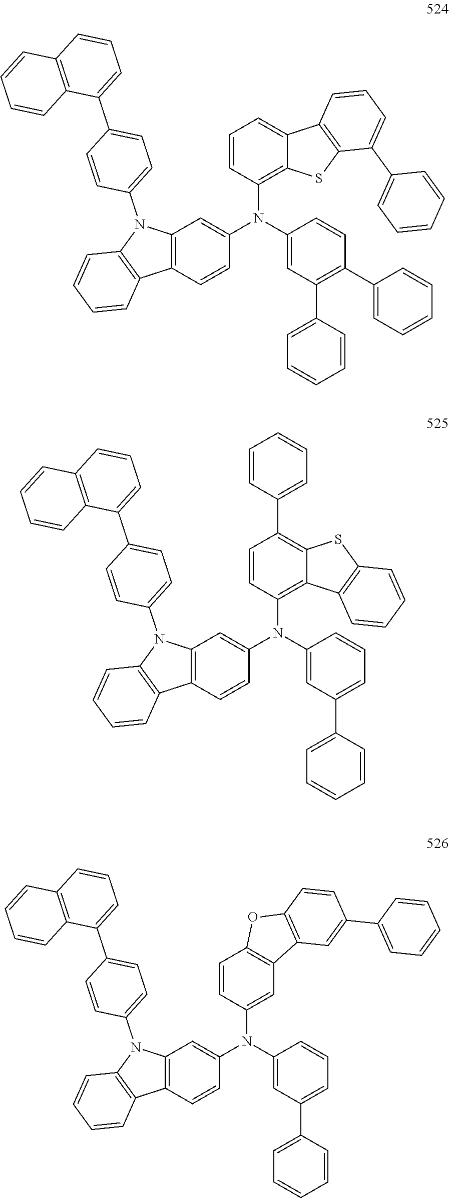 [SMILES: c1ccc(-c2ccc(N(c3ccc4c5ccccc5n(-c5ccc(-c6cccc7ccccc67)cc5)c4c3)c3cccc4c3sc3c(-c5ccccc5)cccc34)cc2-c2ccccc2)cc1 ; c1ccc(-c2cccc(N(c3ccc4c5ccccc5n(-c5ccc(-c6cccc7ccccc67)cc5)c4c3)c3ccc(-c4ccccc4)c4sc5ccccc5c34)c2)cc1 ; c1ccc(-c2cccc(N(c3ccc4oc5ccc(-c6ccccc6)cc5c4c3)c3ccc4c5ccccc5n(-c5ccc(-c6cccc7ccccc67)cc5)c4c3)c2)cc1] 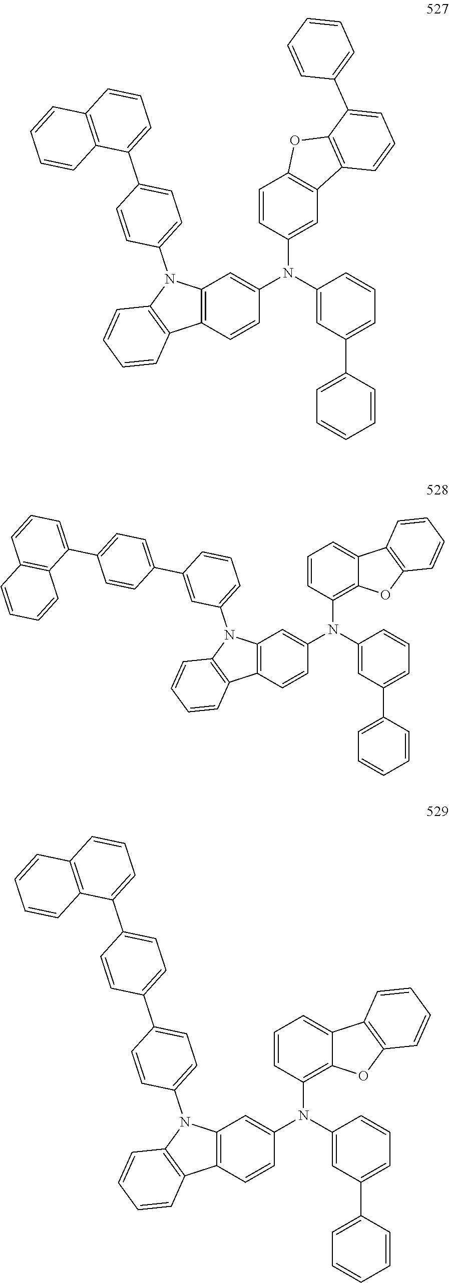 [SMILES: c1ccc(-c2cccc(N(c3ccc4oc5c(-c6ccccc6)cccc5c4c3)c3ccc4c5ccccc5n(-c5ccc(-c6cccc7ccccc67)cc5)c4c3)c2)cc1 ; c1ccc(-c2cccc(N(c3ccc4c5ccccc5n(-c5cccc(-c6ccc(-c7cccc8ccccc78)cc6)c5)c4c3)c3cccc4c3oc3ccccc34)c2)cc1 ; c1ccc(-c2cccc(N(c3ccc4c5ccccc5n(-c5ccc(-c6ccc(-c7cccc8ccccc78)cc6)cc5)c4c3)c3cccc4c3oc3ccccc34)c2)cc1] 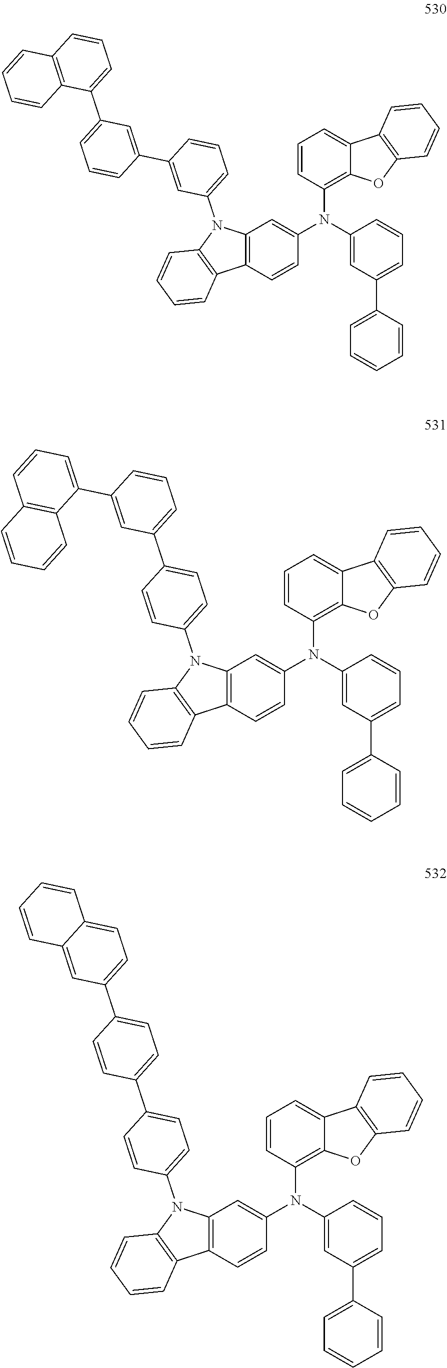 [SMILES: c1ccc(-c2cccc(N(c3ccc4c5ccccc5n(-c5cccc(-c6cccc(-c7cccc8ccccc78)c6)c5)c4c3)c3cccc4c3oc3ccccc34)c2)cc1 ; c1ccc(-c2cccc(N(c3ccc4c5ccccc5n(-c5ccc(-c6cccc(-c7cccc8ccccc78)c6)cc5)c4c3)c3cccc4c3oc3ccccc34)c2)cc1 ; c1ccc(-c2cccc(N(c3ccc4c5ccccc5n(-c5ccc(-c6ccc(-c7ccc8ccccc8c7)cc6)cc5)c4c3)c3cccc4c3oc3ccccc34)c2)cc1] 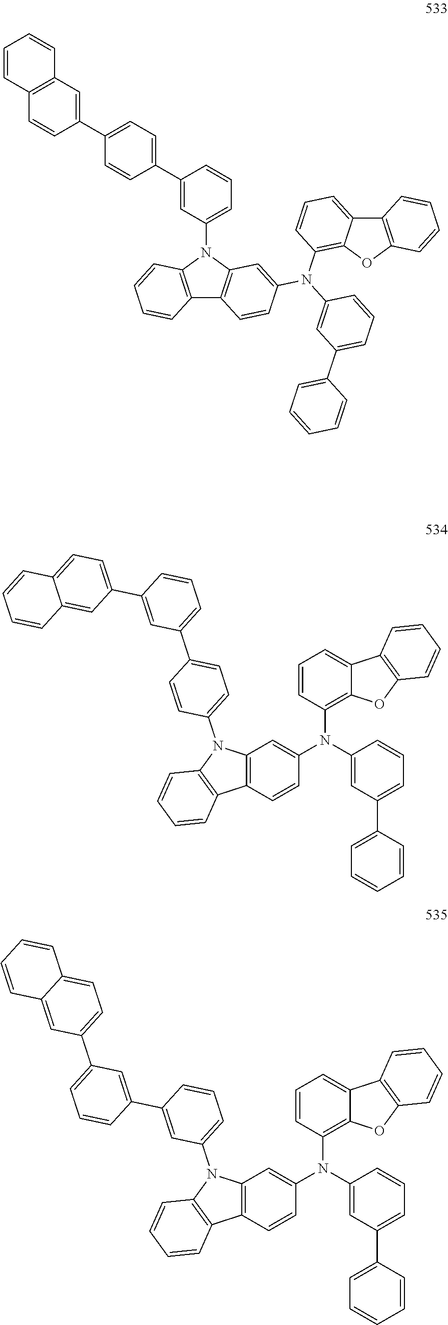 [SMILES: c1ccc(-c2cccc(N(c3ccc4c5ccccc5n(-c5cccc(-c6ccc(-c7ccc8ccccc8c7)cc6)c5)c4c3)c3cccc4c3oc3ccccc34)c2)cc1 ; c1ccc(-c2cccc(N(c3ccc4c5ccccc5n(-c5ccc(-c6cccc(-c7ccc8ccccc8c7)c6)cc5)c4c3)c3cccc4c3oc3ccccc34)c2)cc1 ; c1ccc(-c2cccc(N(c3ccc4c5ccccc5n(-c5cccc(-c6cccc(-c7ccc8ccccc8c7)c6)c5)c4c3)c3cccc4c3oc3ccccc34)c2)cc1] 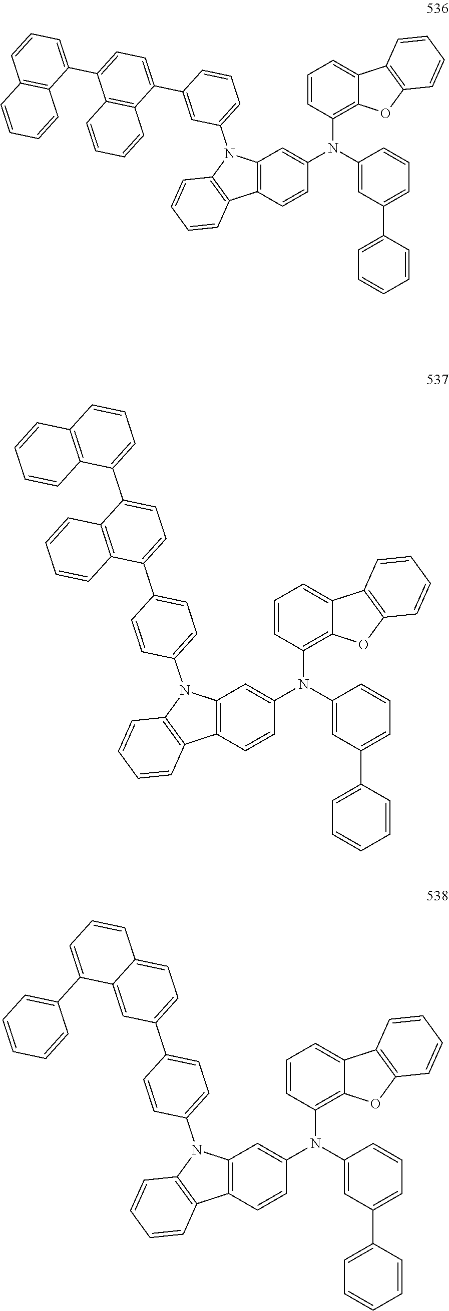 [SMILES: c1ccc(-c2cccc(N(c3ccc4c5ccccc5n(-c5cccc(-c6ccc(-c7cccc8ccccc78)c7ccccc67)c5)c4c3)c3cccc4c3oc3ccccc34)c2)cc1 ; c1ccc(-c2cccc(N(c3ccc4c5ccccc5n(-c5ccc(-c6ccc(-c7cccc8ccccc78)c7ccccc67)cc5)c4c3)c3cccc4c3oc3ccccc34)c2)cc1 ; c1ccc(-c2cccc(N(c3ccc4c5ccccc5n(-c5ccc(-c6ccc7cccc(-c8ccccc8)c7c6)cc5)c4c3)c3cccc4c3oc3ccccc34)c2)cc1] 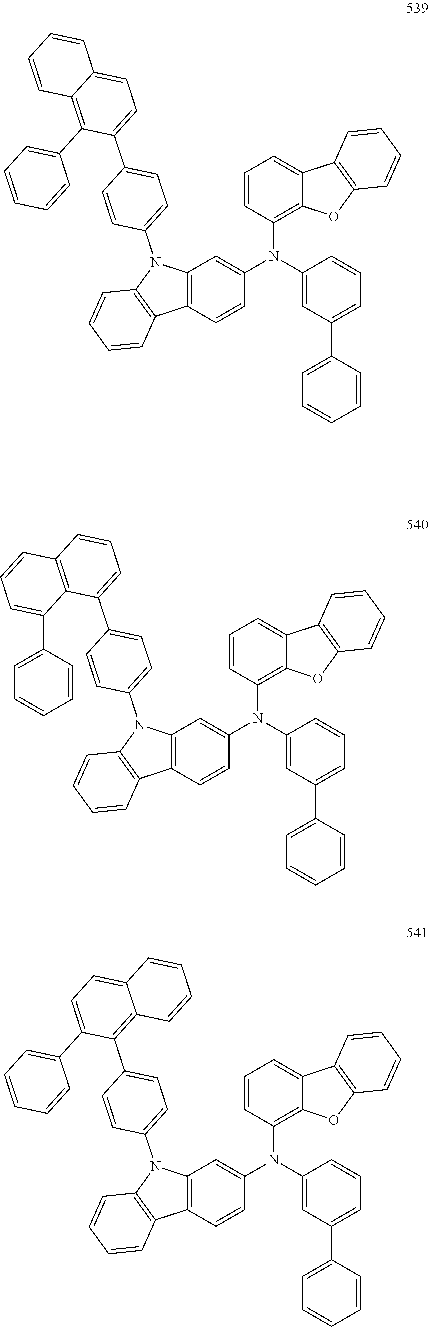 [SMILES: c1ccc(-c2cccc(N(c3ccc4c5ccccc5n(-c5ccc(-c6ccc7ccccc7c6-c6ccccc6)cc5)c4c3)c3cccc4c3oc3ccccc34)c2)cc1 ; c1ccc(-c2cccc(N(c3ccc4c5ccccc5n(-c5ccc(-c6cccc7cccc(-c8ccccc8)c67)cc5)c4c3)c3cccc4c3oc3ccccc34)c2)cc1 ; c1ccc(-c2cccc(N(c3ccc4c5ccccc5n(-c5ccc(-c6c(-c7ccccc7)ccc7ccccc67)cc5)c4c3)c3cccc4c3oc3ccccc34)c2)cc1] 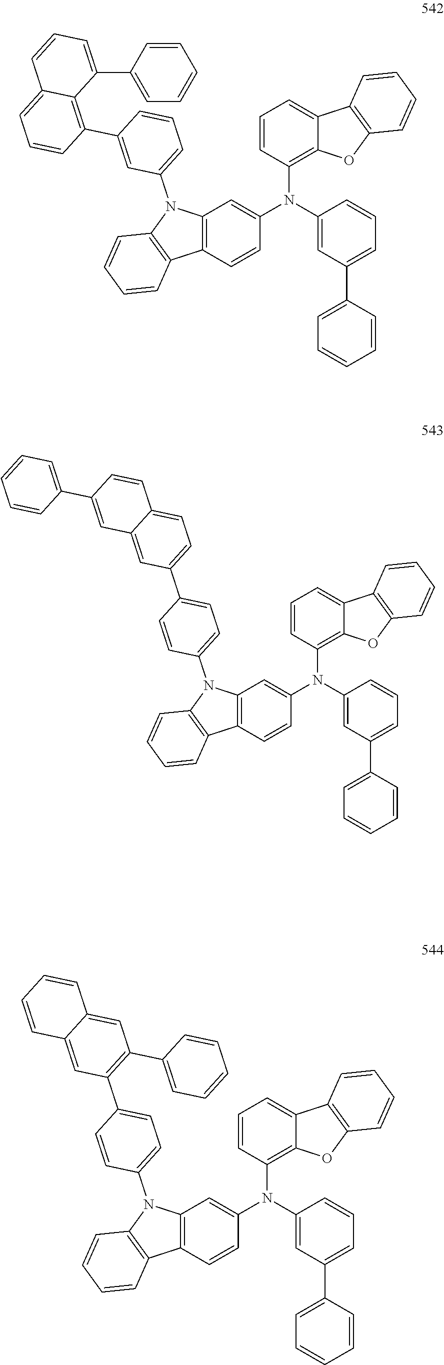 [SMILES: c1ccc(-c2cccc(N(c3ccc4c5ccccc5n(-c5cccc(-c6cccc7cccc(-c8ccccc8)c67)c5)c4c3)c3cccc4c3oc3ccccc34)c2)cc1 ; c1ccc(-c2cccc(N(c3ccc4c5ccccc5n(-c5ccc(-c6ccc7ccc(-c8ccccc8)cc7c6)cc5)c4c3)c3cccc4c3oc3ccccc34)c2)cc1 ; c1ccc(-c2cccc(N(c3ccc4c5ccccc5n(-c5ccc(-c6cc7ccccc7cc6-c6ccccc6)cc5)c4c3)c3cccc4c3oc3ccccc34)c2)cc1] 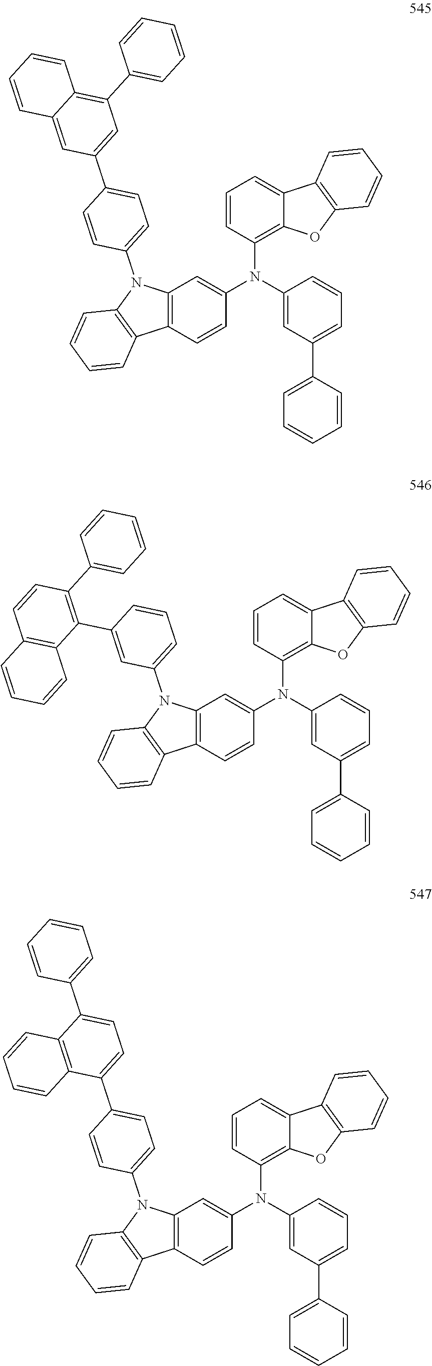 [SMILES: c1ccc(-c2cccc(N(c3ccc4c5ccccc5n(-c5ccc(-c6cc(-c7ccccc7)c7ccccc7c6)cc5)c4c3)c3cccc4c3oc3ccccc34)c2)cc1 ; c1ccc(-c2cccc(N(c3ccc4c5ccccc5n(-c5cccc(-c6c(-c7ccccc7)ccc7ccccc67)c5)c4c3)c3cccc4c3oc3ccccc34)c2)cc1 ; c1ccc(-c2cccc(N(c3ccc4c5ccccc5n(-c5ccc(-c6ccc(-c7ccccc7)c7ccccc67)cc5)c4c3)c3cccc4c3oc3ccccc34)c2)cc1] 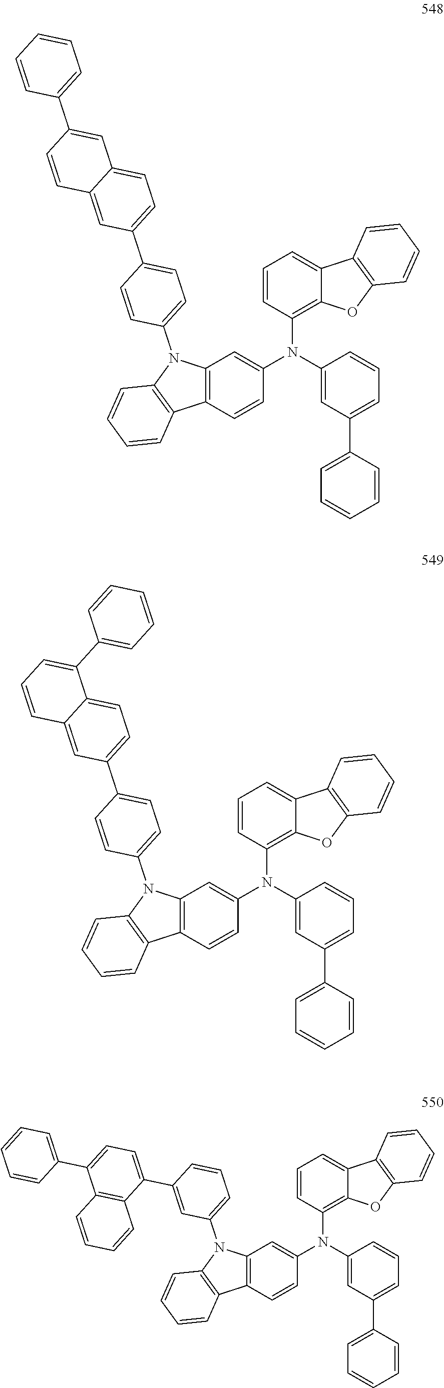 [SMILES: c1ccc(-c2cccc(N(c3ccc4c5ccccc5n(-c5ccc(-c6ccc7cc(-c8ccccc8)ccc7c6)cc5)c4c3)c3cccc4c3oc3ccccc34)c2)cc1 ; c1ccc(-c2cccc(N(c3ccc4c5ccccc5n(-c5ccc(-c6ccc7c(-c8ccccc8)cccc7c6)cc5)c4c3)c3cccc4c3oc3ccccc34)c2)cc1 ; c1ccc(-c2cccc(N(c3ccc4c5ccccc5n(-c5cccc(-c6ccc(-c7ccccc7)c7ccccc67)c5)c4c3)c3cccc4c3oc3ccccc34)c2)cc1] 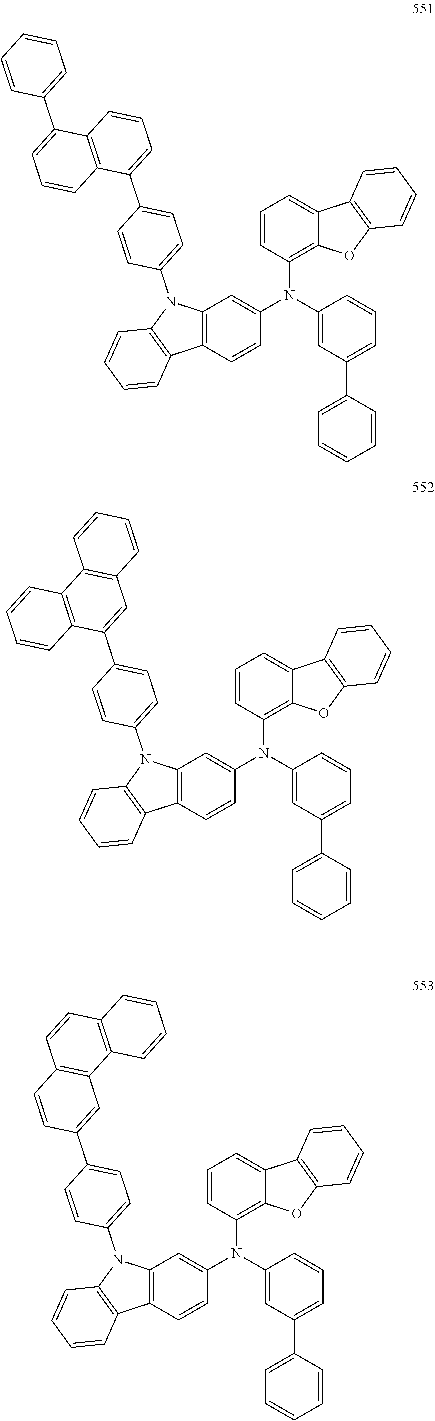 [SMILES: c1ccc(-c2cccc(N(c3ccc4c5ccccc5n(-c5ccc(-c6cccc7c(-c8ccccc8)cccc67)cc5)c4c3)c3cccc4c3oc3ccccc34)c2)cc1 ; c1ccc(-c2cccc(N(c3ccc4c5ccccc5n(-c5ccc(-c6cc7ccccc7c7ccccc67)cc5)c4c3)c3cccc4c3oc3ccccc34)c2)cc1 ; c1ccc(-c2cccc(N(c3ccc4c5ccccc5n(-c5ccc(-c6ccc7ccc8ccccc8c7c6)cc5)c4c3)c3cccc4c3oc3ccccc34)c2)cc1] 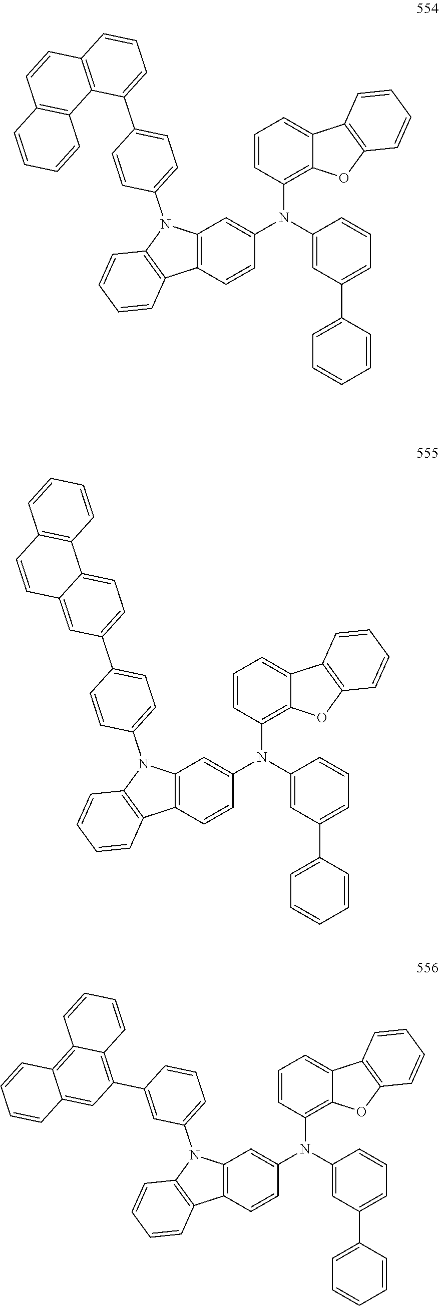 [SMILES: c1ccc(-c2cccc(N(c3ccc4c5ccccc5n(-c5ccc(-c6cccc7ccc8ccccc8c67)cc5)c4c3)c3cccc4c3oc3ccccc34)c2)cc1 ; c1ccc(-c2cccc(N(c3ccc4c5ccccc5n(-c5ccc(-c6ccc7c(ccc8ccccc87)c6)cc5)c4c3)c3cccc4c3oc3ccccc34)c2)cc1 ; c1ccc(-c2cccc(N(c3ccc4c5ccccc5n(-c5cccc(-c6cc7ccccc7c7ccccc67)c5)c4c3)c3cccc4c3oc3ccccc34)c2)cc1] 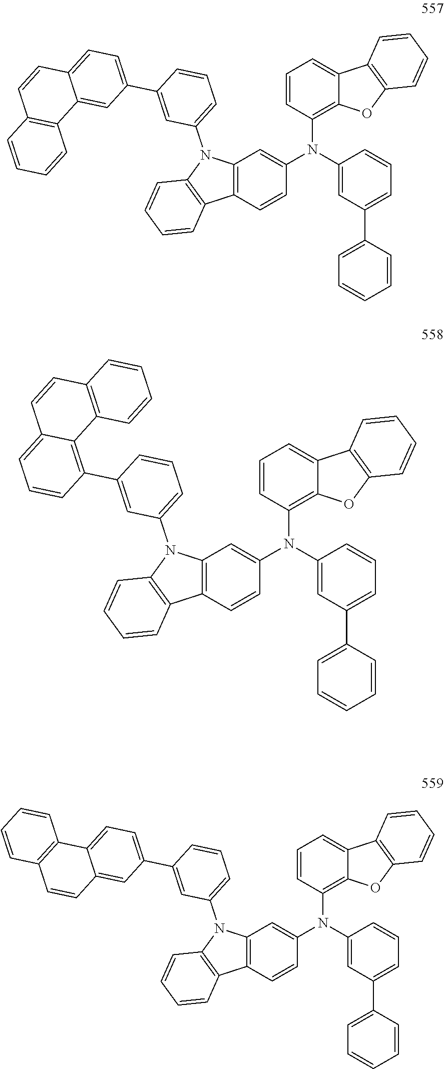 [SMILES: c1ccc(-c2cccc(N(c3ccc4c5ccccc5n(-c5cccc(-c6ccc7ccc8ccccc8c7c6)c5)c4c3)c3cccc4c3oc3ccccc34)c2)cc1 ; c1ccc(-c2cccc(N(c3ccc4c5ccccc5n(-c5cccc(-c6cccc7ccc8ccccc8c67)c5)c4c3)c3cccc4c3oc3ccccc34)c2)cc1 ; c1ccc(-c2cccc(N(c3ccc4c5ccccc5n(-c5cccc(-c6ccc7c(ccc8ccccc87)c6)c5)c4c3)c3cccc4c3oc3ccccc34)c2)cc1] 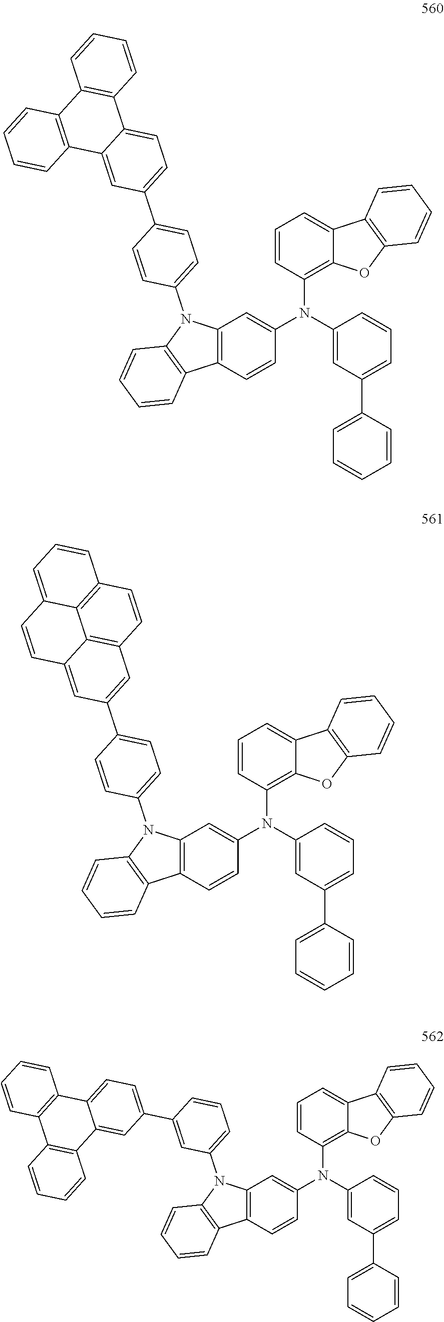 [SMILES: c1ccc(-c2cccc(N(c3ccc4c5ccccc5n(-c5ccc(-c6ccc7c8ccccc8c8ccccc8c7c6)cc5)c4c3)c3cccc4c3oc3ccccc34)c2)cc1 ; c1ccc(-c2cccc(N(c3ccc4c5ccccc5n(-c5ccc(-c6cc7ccc8cccc9ccc(c6)c7c89)cc5)c4c3)c3cccc4c3oc3ccccc34)c2)cc1 ; c1ccc(-c2cccc(N(c3ccc4c5ccccc5n(-c5cccc(-c6ccc7c8ccccc8c8ccccc8c7c6)c5)c4c3)c3cccc4c3oc3ccccc34)c2)cc1] 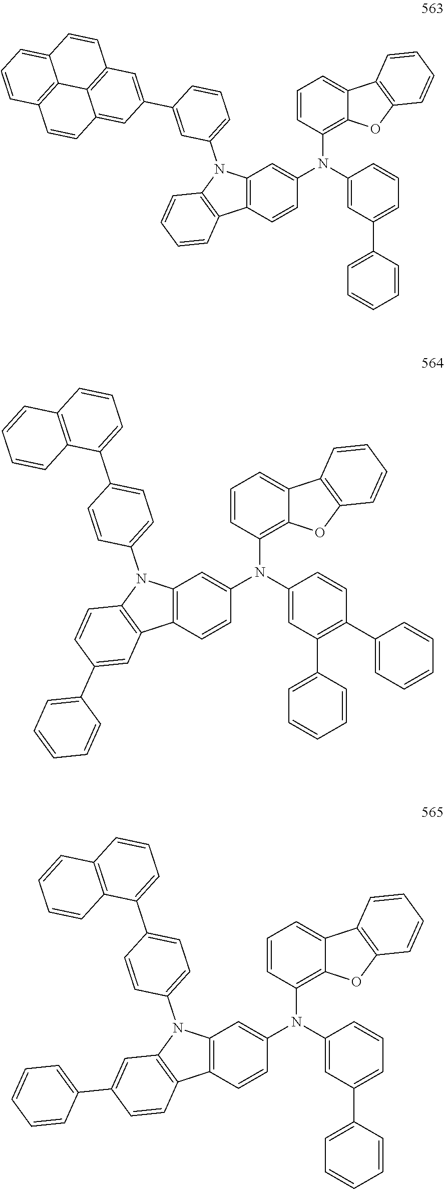 [SMILES: c1ccc(-c2cccc(N(c3ccc4c5ccccc5n(-c5cccc(-c6cc7ccc8cccc9ccc(c6)c7c89)c5)c4c3)c3cccc4c3oc3ccccc34)c2)cc1 ; c1ccc(-c2ccc3c(c2)c2ccc(N(c4ccc(-c5ccccc5)c(-c5ccccc5)c4)c4cccc5c4oc4ccccc45)cc2n3-c2ccc(-c3cccc4ccccc34)cc2)cc1 ; c1ccc(-c2cccc(N(c3ccc4c5ccc(-c6ccccc6)cc5n(-c5ccc(-c6cccc7ccccc67)cc5)c4c3)c3cccc4c3oc3ccccc34)c2)cc1] 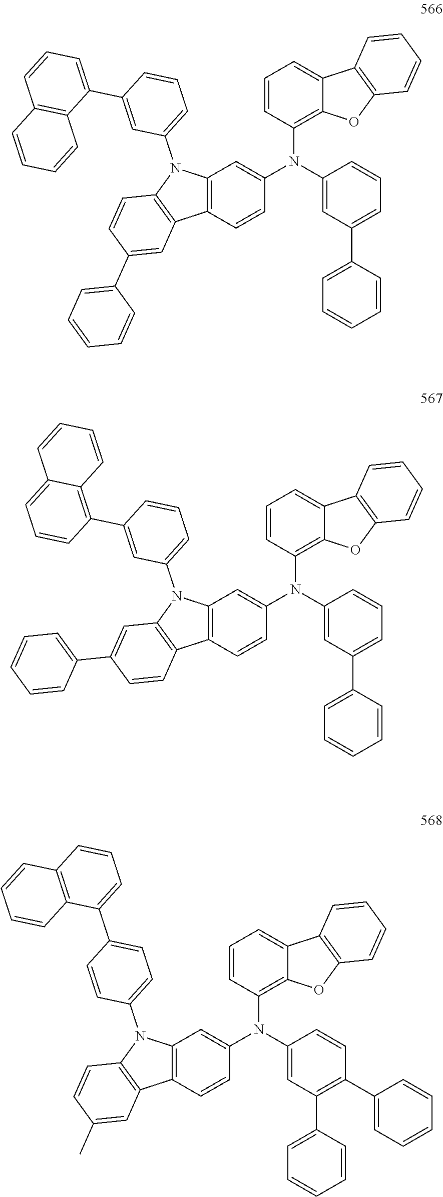 [SMILES: c1ccc(-c2cccc(N(c3ccc4c5cc(-c6ccccc6)ccc5n(-c5cccc(-c6cccc7ccccc67)c5)c4c3)c3cccc4c3oc3ccccc34)c2)cc1 ; c1ccc(-c2cccc(N(c3ccc4c5ccc(-c6ccccc6)cc5n(-c5cccc(-c6cccc7ccccc67)c5)c4c3)c3cccc4c3oc3ccccc34)c2)cc1 ; Cc1ccc2c(c1)c1ccc(N(c3ccc(-c4ccccc4)c(-c4ccccc4)c3)c3cccc4c3oc3ccccc34)cc1n2-c1ccc(-c2cccc3ccccc23)cc1] 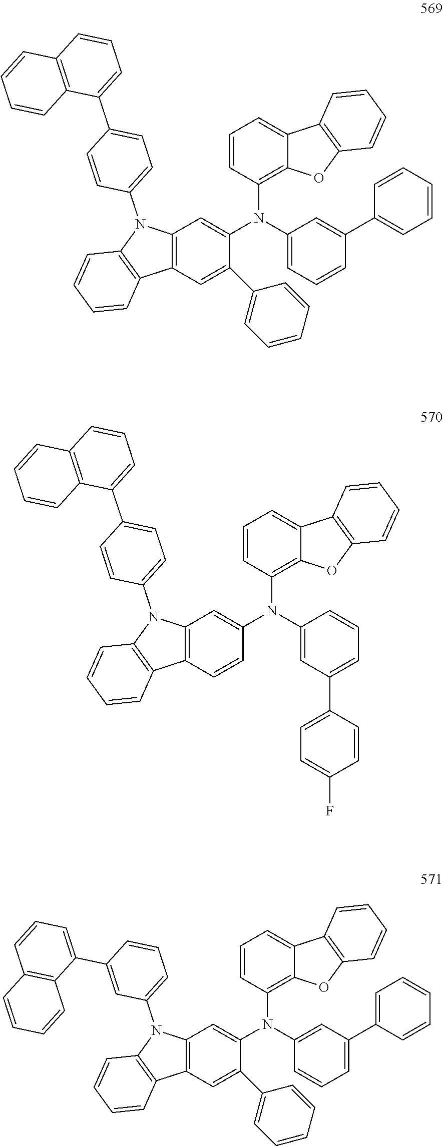 [SMILES: c1ccc(-c2cccc(N(c3cc4c(cc3-c3ccccc3)c3ccccc3n4-c3ccc(-c4cccc5ccccc45)cc3)c3cccc4c3oc3ccccc34)c2)cc1 ; Fc1ccc(-c2cccc(N(c3ccc4c5ccccc5n(-c5ccc(-c6cccc7ccccc67)cc5)c4c3)c3cccc4c3oc3ccccc34)c2)cc1 ; c1ccc(-c2cccc(N(c3cc4c(cc3-c3ccccc3)c3ccccc3n4-c3cccc(-c4cccc5ccccc45)c3)c3cccc4c3oc3ccccc34)c2)cc1] 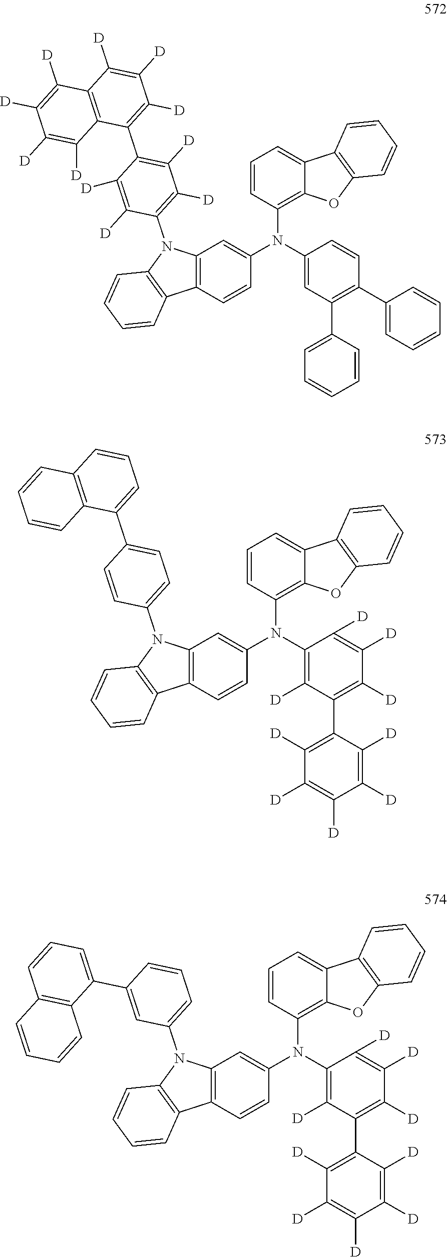 [SMILES: [2H]c1c([2H])c(-n2c3ccccc3c3ccc(N(c4ccc(-c5ccccc5)c(-c5ccccc5)c4)c4cccc5c4oc4ccccc45)cc32)c([2H])c([2H])c1-c1c([2H])c([2H])c([2H])c2c([2H])c([2H])c([2H])c([2H])c12 ; [2H]c1c([2H])c([2H])c(-c2c([2H])c([2H])c([2H])c(N(c3ccc4c5ccccc5n(-c5ccc(-c6cccc7ccccc67)cc5)c4c3)c3cccc4c3oc3ccccc34)c2[2H])c([2H])c1[2H] ; [2H]c1c([2H])c([2H])c(-c2c([2H])c([2H])c([2H])c(N(c3ccc4c5ccccc5n(-c5cccc(-c6cccc7ccccc67)c5)c4c3)c3cccc4c3oc3ccccc34)c2[2H])c([2H])c1[2H]] 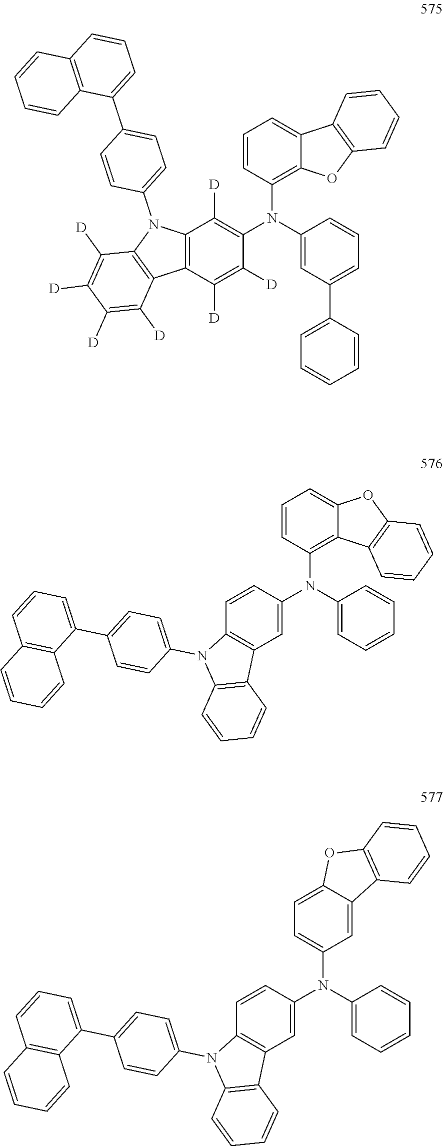 [SMILES: [2H]c1c([2H])c([2H])c2c(c1[2H])c1c([2H])c([2H])c(N(c3cccc(-c4ccccc4)c3)c3cccc4c3oc3ccccc34)c([2H])c1n2-c1ccc(-c2cccc3ccccc23)cc1 ; c1ccc(N(c2ccc3c(c2)c2ccccc2n3-c2ccc(-c3cccc4ccccc34)cc2)c2cccc3oc4ccccc4c23)cc1 ; c1ccc(N(c2ccc3oc4ccccc4c3c2)c2ccc3c(c2)c2ccccc2n3-c2ccc(-c3cccc4ccccc34)cc2)cc1] 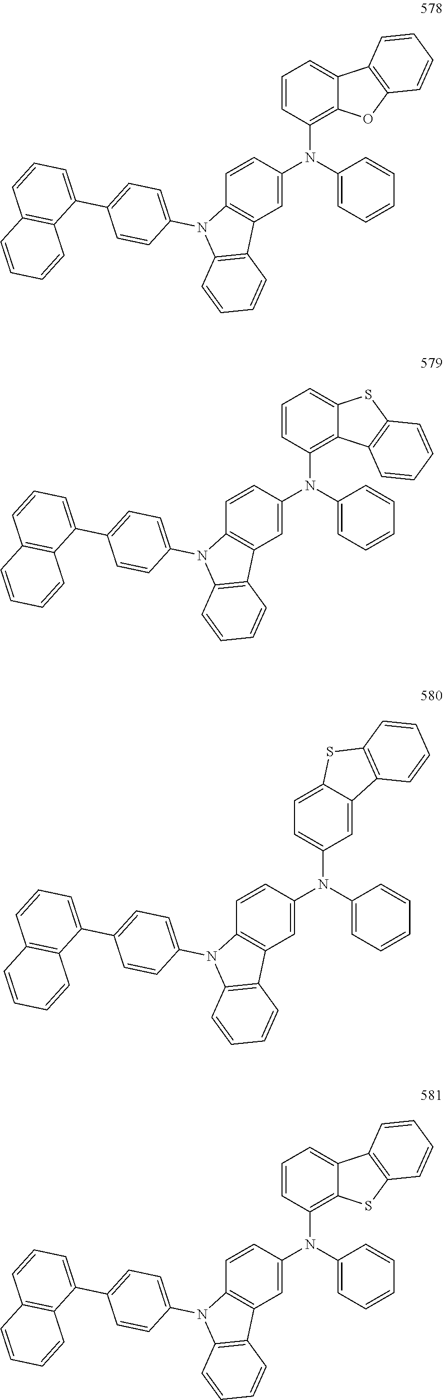 [SMILES: c1ccc(N(c2ccc3c(c2)c2ccccc2n3-c2ccc(-c3cccc4ccccc34)cc2)c2cccc3c2oc2ccccc23)cc1 ; c1ccc(N(c2ccc3c(c2)c2ccccc2n3-c2ccc(-c3cccc4ccccc34)cc2)c2cccc3sc4ccccc4c23)cc1 ; c1ccc(N(c2ccc3sc4ccccc4c3c2)c2ccc3c(c2)c2ccccc2n3-c2ccc(-c3cccc4ccccc34)cc2)cc1 ; c1ccc(N(c2ccc3c(c2)c2ccccc2n3-c2ccc(-c3cccc4ccccc34)cc2)c2cccc3c2sc2ccccc23)cc1] 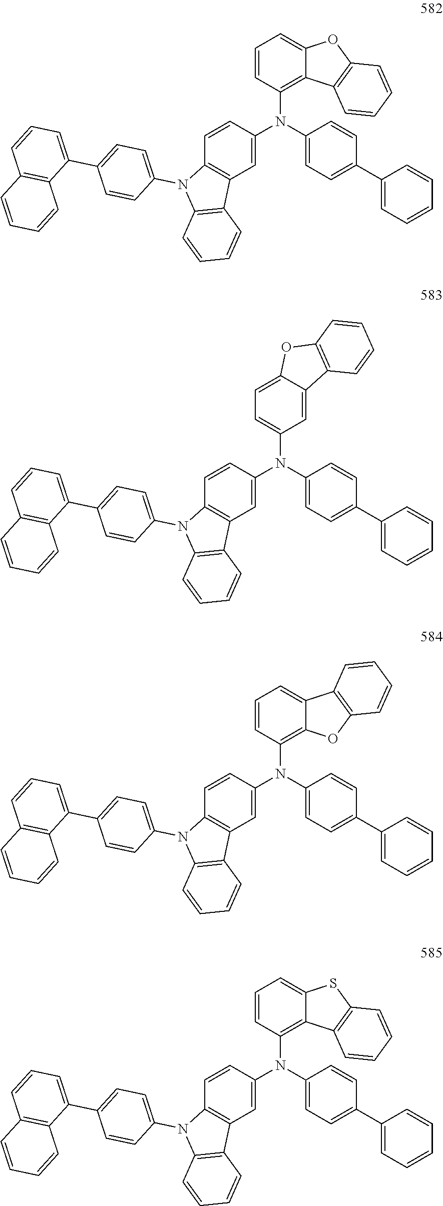 [SMILES: c1ccc(-c2ccc(N(c3ccc4c(c3)c3ccccc3n4-c3ccc(-c4cccc5ccccc45)cc3)c3cccc4oc5ccccc5c34)cc2)cc1 ; c1ccc(-c2ccc(N(c3ccc4oc5ccccc5c4c3)c3ccc4c(c3)c3ccccc3n4-c3ccc(-c4cccc5ccccc45)cc3)cc2)cc1 ; c1ccc(-c2ccc(N(c3ccc4c(c3)c3ccccc3n4-c3ccc(-c4cccc5ccccc45)cc3)c3cccc4c3oc3ccccc34)cc2)cc1 ; c1ccc(-c2ccc(N(c3ccc4c(c3)c3ccccc3n4-c3ccc(-c4cccc5ccccc45)cc3)c3cccc4sc5ccccc5c34)cc2)cc1] 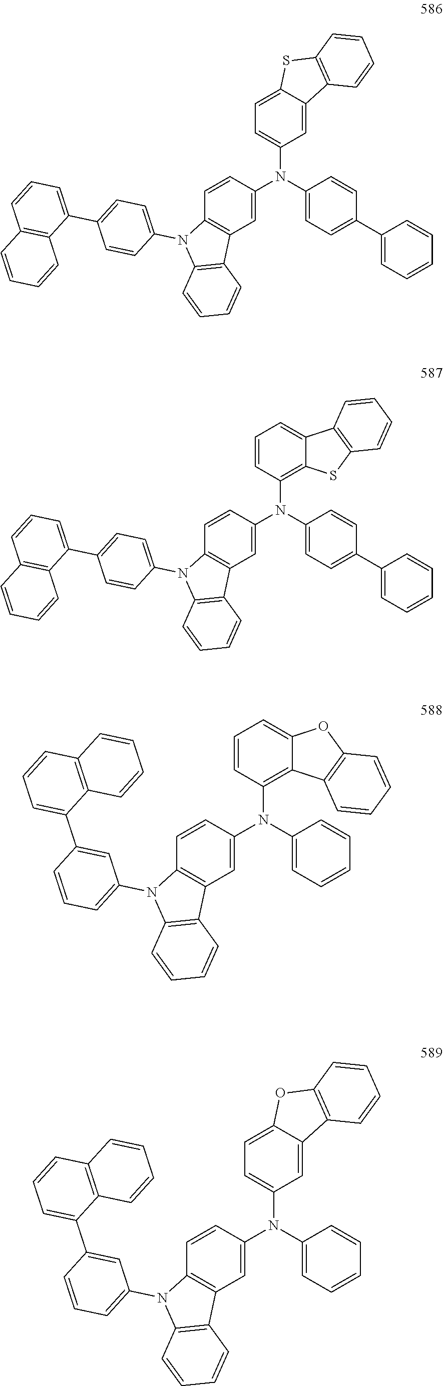 [SMILES: c1ccc(-c2ccc(N(c3ccc4sc5ccccc5c4c3)c3ccc4c(c3)c3ccccc3n4-c3ccc(-c4cccc5ccccc45)cc3)cc2)cc1 ; c1ccc(-c2ccc(N(c3ccc4c(c3)c3ccccc3n4-c3ccc(-c4cccc5ccccc45)cc3)c3cccc4c3sc3ccccc34)cc2)cc1 ; c1ccc(N(c2ccc3c(c2)c2ccccc2n3-c2cccc(-c3cccc4ccccc34)c2)c2cccc3oc4ccccc4c23)cc1 ; c1ccc(N(c2ccc3oc4ccccc4c3c2)c2ccc3c(c2)c2ccccc2n3-c2cccc(-c3cccc4ccccc34)c2)cc1] 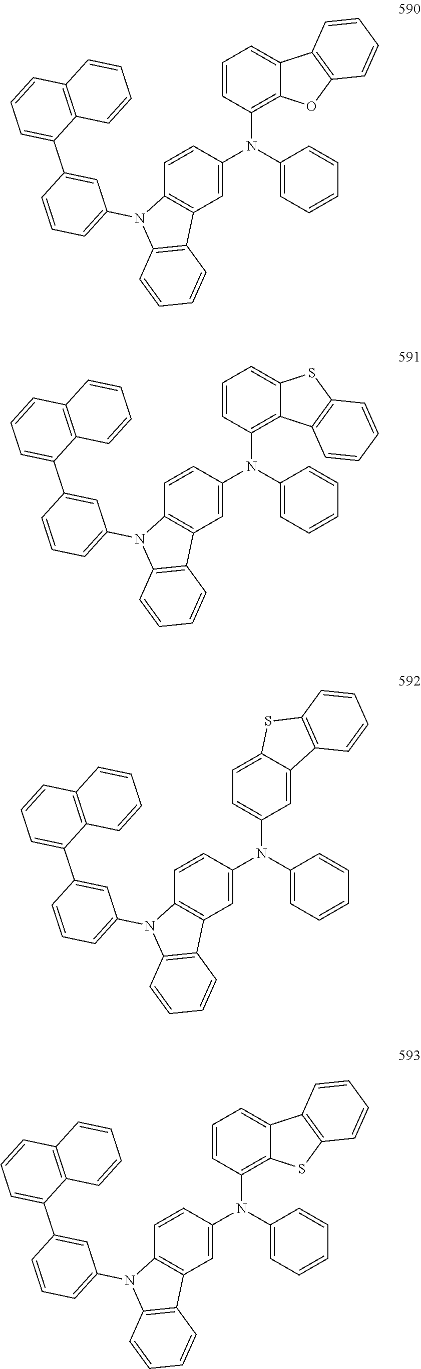 [SMILES: c1ccc(N(c2ccc3c(c2)c2ccccc2n3-c2cccc(-c3cccc4ccccc34)c2)c2cccc3c2oc2ccccc23)cc1 ; c1ccc(N(c2ccc3c(c2)c2ccccc2n3-c2cccc(-c3cccc4ccccc34)c2)c2cccc3sc4ccccc4c23)cc1 ; c1ccc(N(c2ccc3sc4ccccc4c3c2)c2ccc3c(c2)c2ccccc2n3-c2cccc(-c3cccc4ccccc34)c2)cc1 ; c1ccc(N(c2ccc3c(c2)c2ccccc2n3-c2cccc(-c3cccc4ccccc34)c2)c2cccc3c2sc2ccccc23)cc1] 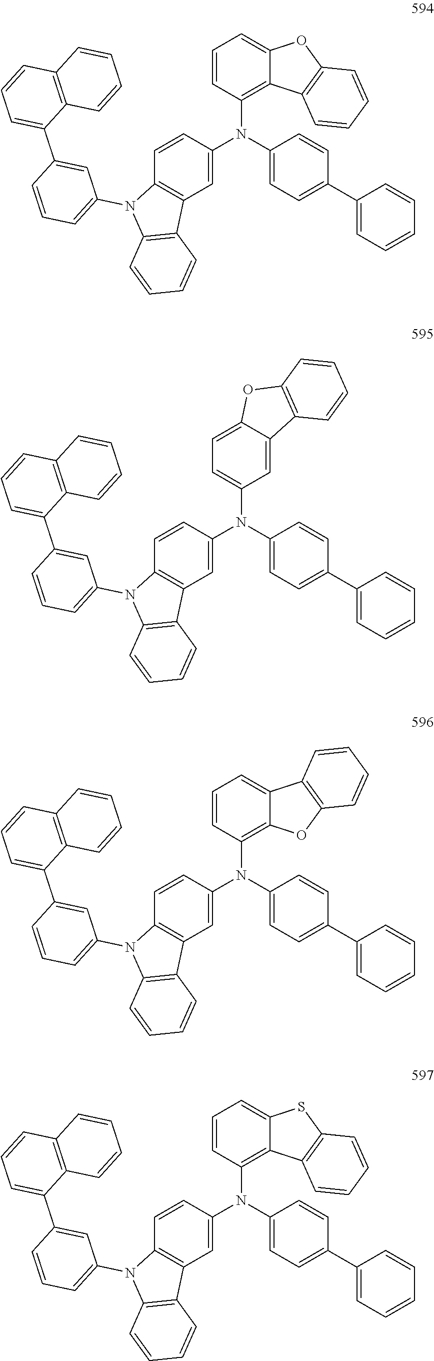 [SMILES: c1ccc(-c2ccc(N(c3ccc4c(c3)c3ccccc3n4-c3cccc(-c4cccc5ccccc45)c3)c3cccc4oc5ccccc5c34)cc2)cc1 ; c1ccc(-c2ccc(N(c3ccc4oc5ccccc5c4c3)c3ccc4c(c3)c3ccccc3n4-c3cccc(-c4cccc5ccccc45)c3)cc2)cc1 ; c1ccc(-c2ccc(N(c3ccc4c(c3)c3ccccc3n4-c3cccc(-c4cccc5ccccc45)c3)c3cccc4c3oc3ccccc34)cc2)cc1 ; c1ccc(-c2ccc(N(c3ccc4c(c3)c3ccccc3n4-c3cccc(-c4cccc5ccccc45)c3)c3cccc4sc5ccccc5c34)cc2)cc1] 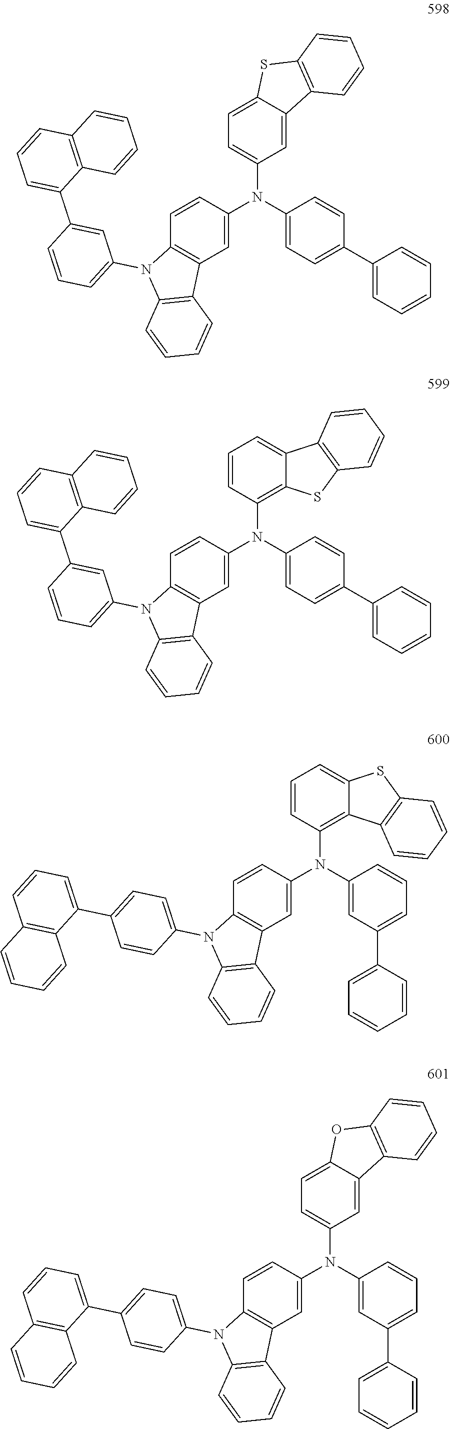 [SMILES: c1ccc(-c2ccc(N(c3ccc4sc5ccccc5c4c3)c3ccc4c(c3)c3ccccc3n4-c3cccc(-c4cccc5ccccc45)c3)cc2)cc1 ; c1ccc(-c2ccc(N(c3ccc4c(c3)c3ccccc3n4-c3cccc(-c4cccc5ccccc45)c3)c3cccc4c3sc3ccccc34)cc2)cc1 ; c1ccc(-c2cccc(N(c3ccc4c(c3)c3ccccc3n4-c3ccc(-c4cccc5ccccc45)cc3)c3cccc4sc5ccccc5c34)c2)cc1 ; c1ccc(-c2cccc(N(c3ccc4oc5ccccc5c4c3)c3ccc4c(c3)c3ccccc3n4-c3ccc(-c4cccc5ccccc45)cc3)c2)cc1] 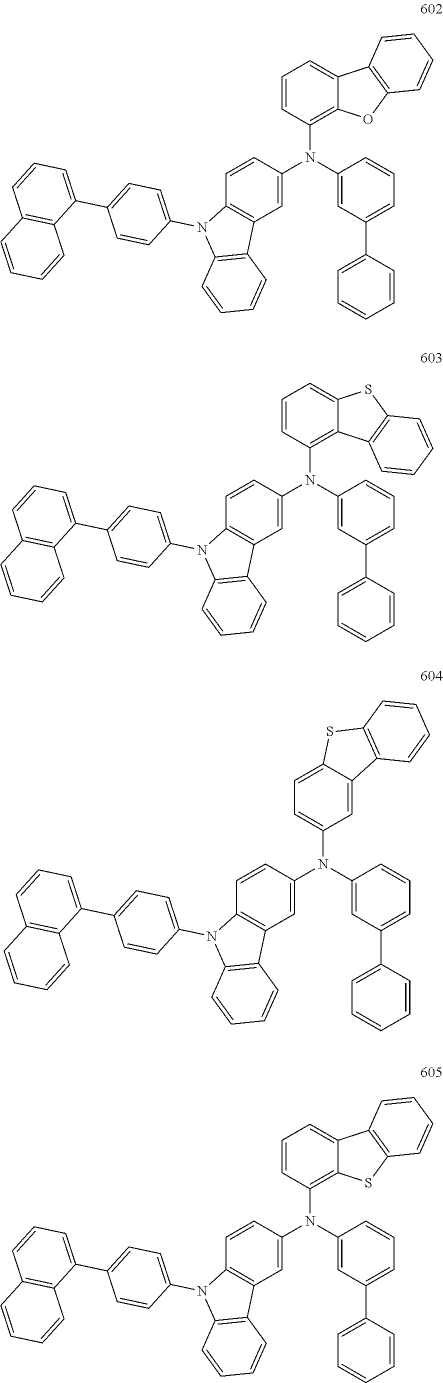 [SMILES: c1ccc(-c2cccc(N(c3ccc4c(c3)c3ccccc3n4-c3ccc(-c4cccc5ccccc45)cc3)c3cccc4c3oc3ccccc34)c2)cc1 ; c1ccc(-c2cccc(N(c3ccc4c(c3)c3ccccc3n4-c3ccc(-c4cccc5ccccc45)cc3)c3cccc4sc5ccccc5c34)c2)cc1 ; c1ccc(-c2cccc(N(c3ccc4sc5ccccc5c4c3)c3ccc4c(c3)c3ccccc3n4-c3ccc(-c4cccc5ccccc45)cc3)c2)cc1 ; c1ccc(-c2cccc(N(c3ccc4c(c3)c3ccccc3n4-c3ccc(-c4cccc5ccccc45)cc3)c3cccc4c3sc3ccccc34)c2)cc1] 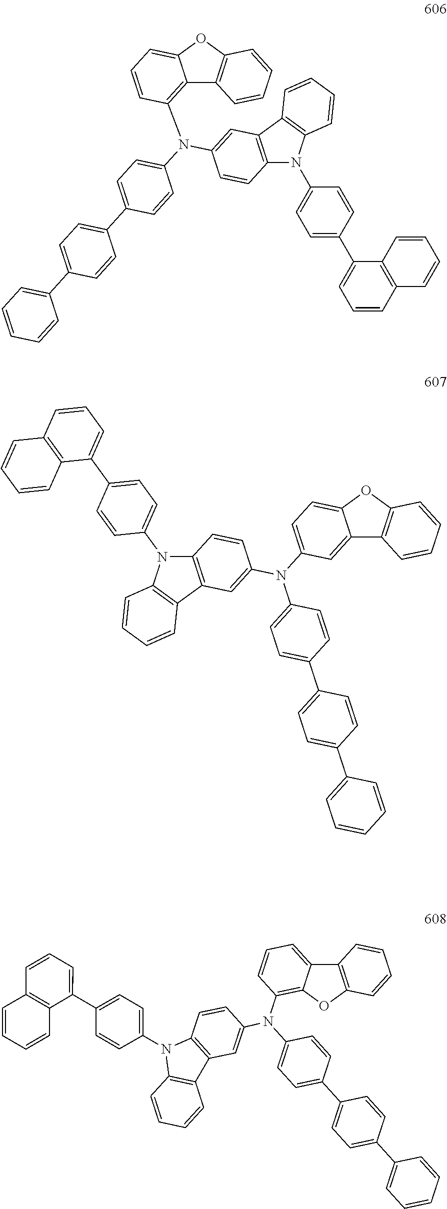 [SMILES: c1ccc(-c2ccc(-c3ccc(N(c4ccc5c(c4)c4ccccc4n5-c4ccc(-c5cccc6ccccc56)cc4)c4cccc5oc6ccccc6c45)cc3)cc2)cc1 ; c1ccc(-c2ccc(-c3ccc(N(c4ccc5oc6ccccc6c5c4)c4ccc5c(c4)c4ccccc4n5-c4ccc(-c5cccc6ccccc56)cc4)cc3)cc2)cc1 ; c1ccc(-c2ccc(-c3ccc(N(c4ccc5c(c4)c4ccccc4n5-c4ccc(-c5cccc6ccccc56)cc4)c4cccc5c4oc4ccccc45)cc3)cc2)cc1] 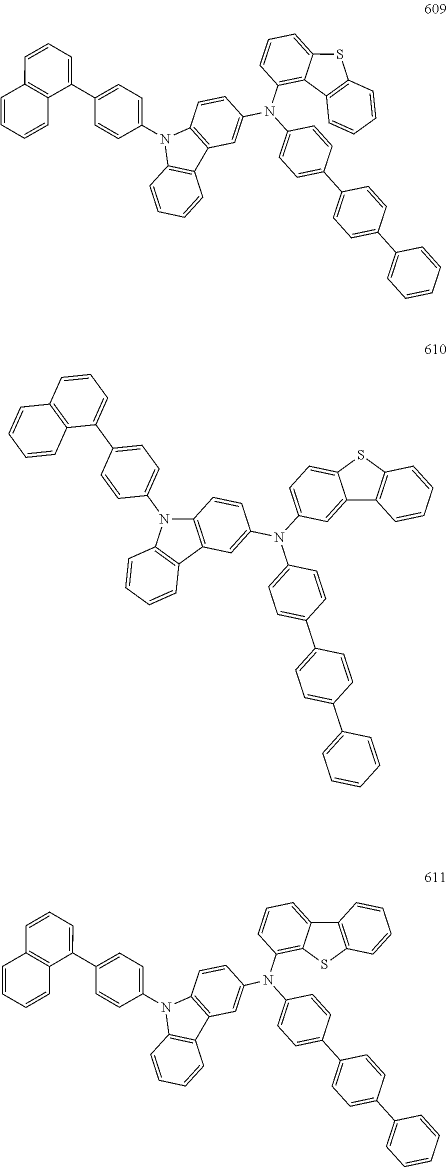 [SMILES: c1ccc(-c2ccc(-c3ccc(N(c4ccc5c(c4)c4ccccc4n5-c4ccc(-c5cccc6ccccc56)cc4)c4cccc5sc6ccccc6c45)cc3)cc2)cc1 ; c1ccc(-c2ccc(-c3ccc(N(c4ccc5sc6ccccc6c5c4)c4ccc5c(c4)c4ccccc4n5-c4ccc(-c5cccc6ccccc56)cc4)cc3)cc2)cc1 ; c1ccc(-c2ccc(-c3ccc(N(c4ccc5c(c4)c4ccccc4n5-c4ccc(-c5cccc6ccccc56)cc4)c4cccc5c4sc4ccccc45)cc3)cc2)cc1] 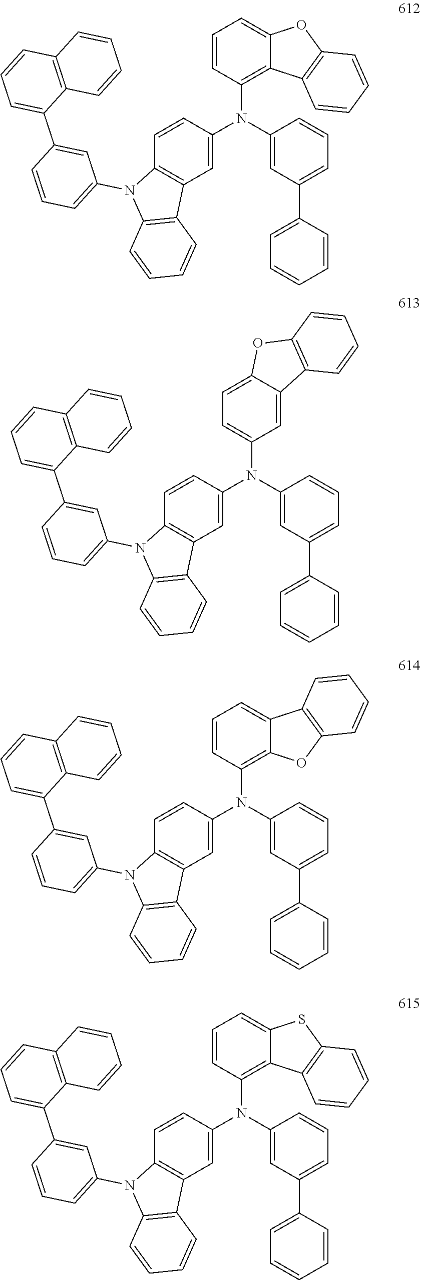 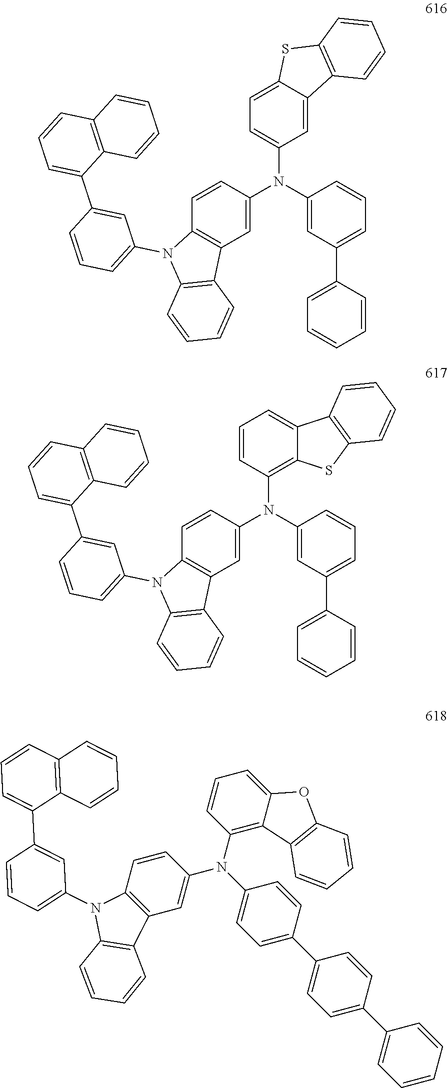 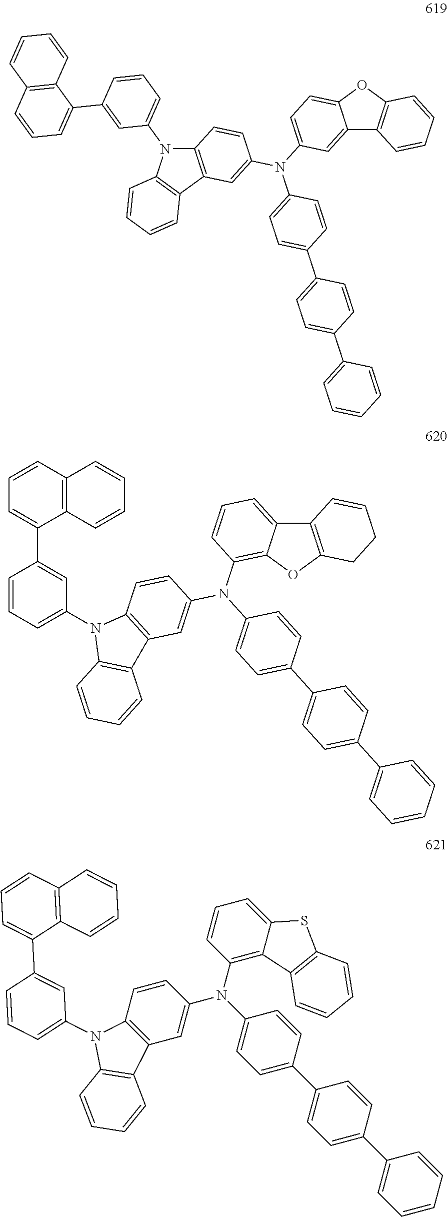 [SMILES: c1ccc(-c2ccc(-c3ccc(N(c4ccc5oc6ccccc6c5c4)c4ccc5c(c4)c4ccccc4n5-c4cccc(-c5cccc6ccccc56)c4)cc3)cc2)cc1 ; C1=Cc2c(oc3c(N(c4ccc(-c5ccc(-c6ccccc6)cc5)cc4)c4ccc5c(c4)c4ccccc4n5-c4cccc(-c5cccc6ccccc56)c4)cccc23)CC1 ; c1ccc(-c2ccc(-c3ccc(N(c4ccc5c(c4)c4ccccc4n5-c4cccc(-c5cccc6ccccc56)c4)c4cccc5sc6ccccc6c45)cc3)cc2)cc1] 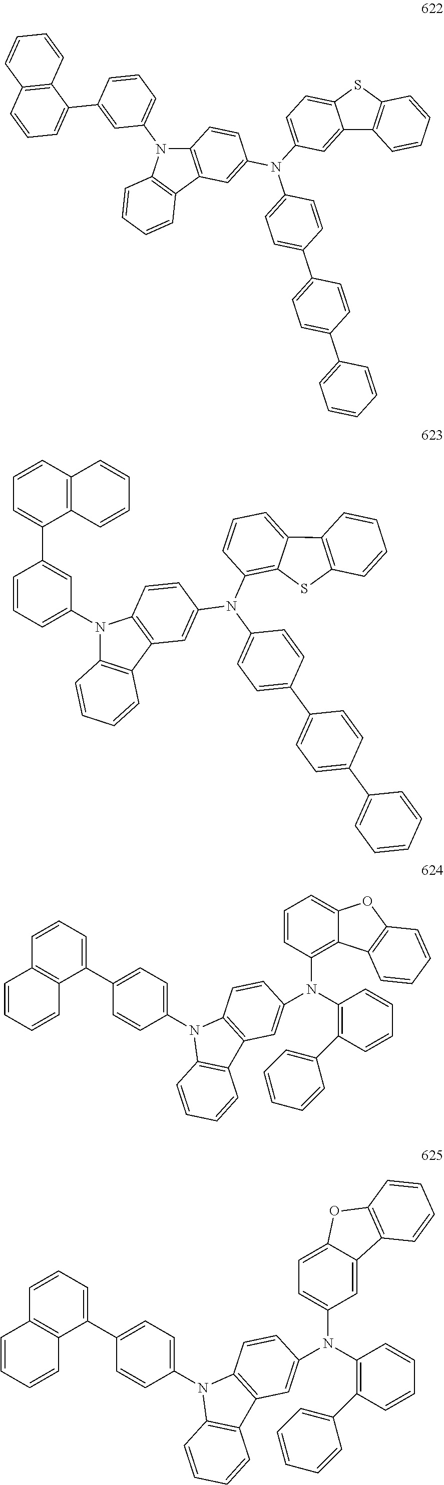 [SMILES: c1ccc(-c2ccc(-c3ccc(N(c4ccc5sc6ccccc6c5c4)c4ccc5c(c4)c4ccccc4n5-c4cccc(-c5cccc6ccccc56)c4)cc3)cc2)cc1 ; c1ccc(-c2ccc(-c3ccc(N(c4ccc5c(c4)c4ccccc4n5-c4cccc(-c5cccc6ccccc56)c4)c4cccc5c4sc4ccccc45)cc3)cc2)cc1 ; c1ccc(-c2ccccc2N(c2ccc3c(c2)c2ccccc2n3-c2ccc(-c3cccc4ccccc34)cc2)c2cccc3oc4ccccc4c23)cc1 ; c1ccc(-c2ccccc2N(c2ccc3oc4ccccc4c3c2)c2ccc3c(c2)c2ccccc2n3-c2ccc(-c3cccc4ccccc34)cc2)cc1] 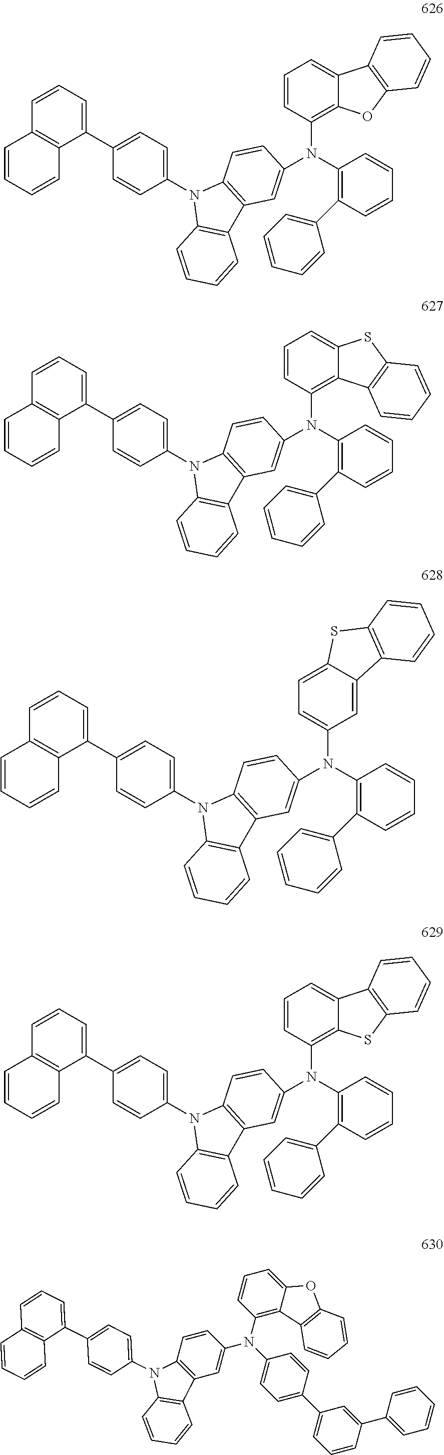 [SMILES: c1ccc(-c2ccccc2N(c2ccc3c(c2)c2ccccc2n3-c2ccc(-c3cccc4ccccc34)cc2)c2cccc3c2oc2ccccc23)cc1 ; c1ccc(-c2ccccc2N(c2ccc3c(c2)c2ccccc2n3-c2ccc(-c3cccc4ccccc34)cc2)c2cccc3sc4ccccc4c23)cc1 ; c1ccc(-c2ccccc2N(c2ccc3sc4ccccc4c3c2)c2ccc3c(c2)c2ccccc2n3-c2ccc(-c3cccc4ccccc34)cc2)cc1 ; c1ccc(-c2ccccc2N(c2ccc3c(c2)c2ccccc2n3-c2ccc(-c3cccc4ccccc34)cc2)c2cccc3c2sc2ccccc23)cc1 ; c1ccc(-c2cccc(-c3ccc(N(c4ccc5c(c4)c4ccccc4n5-c4ccc(-c5cccc6ccccc56)cc4)c4cccc5oc6ccccc6c45)cc3)c2)cc1] 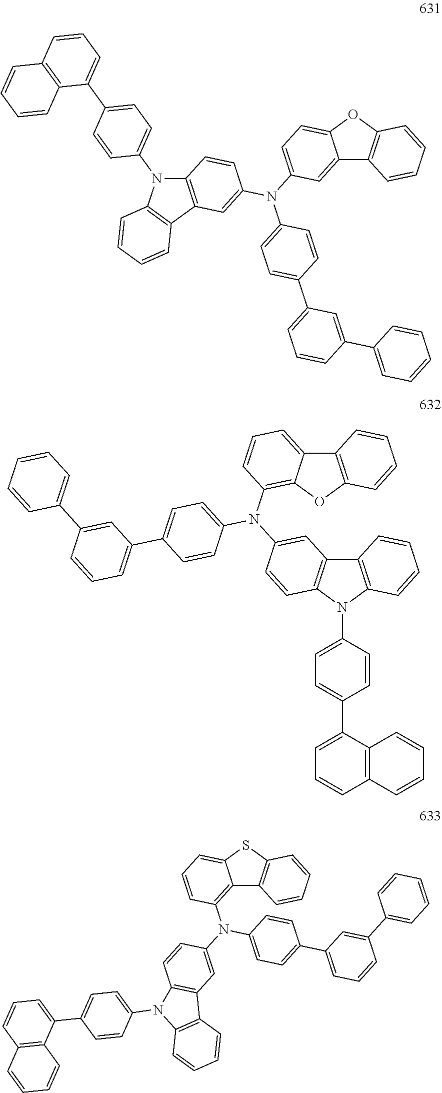 [SMILES: c1ccc(-c2cccc(-c3ccc(N(c4ccc5oc6ccccc6c5c4)c4ccc5c(c4)c4ccccc4n5-c4ccc(-c5cccc6ccccc56)cc4)cc3)c2)cc1 ; c1ccc(-c2cccc(-c3ccc(N(c4ccc5c(c4)c4ccccc4n5-c4ccc(-c5cccc6ccccc56)cc4)c4cccc5c4oc4ccccc45)cc3)c2)cc1 ; c1ccc(-c2cccc(-c3ccc(N(c4ccc5c(c4)c4ccccc4n5-c4ccc(-c5cccc6ccccc56)cc4)c4cccc5sc6ccccc6c45)cc3)c2)cc1] 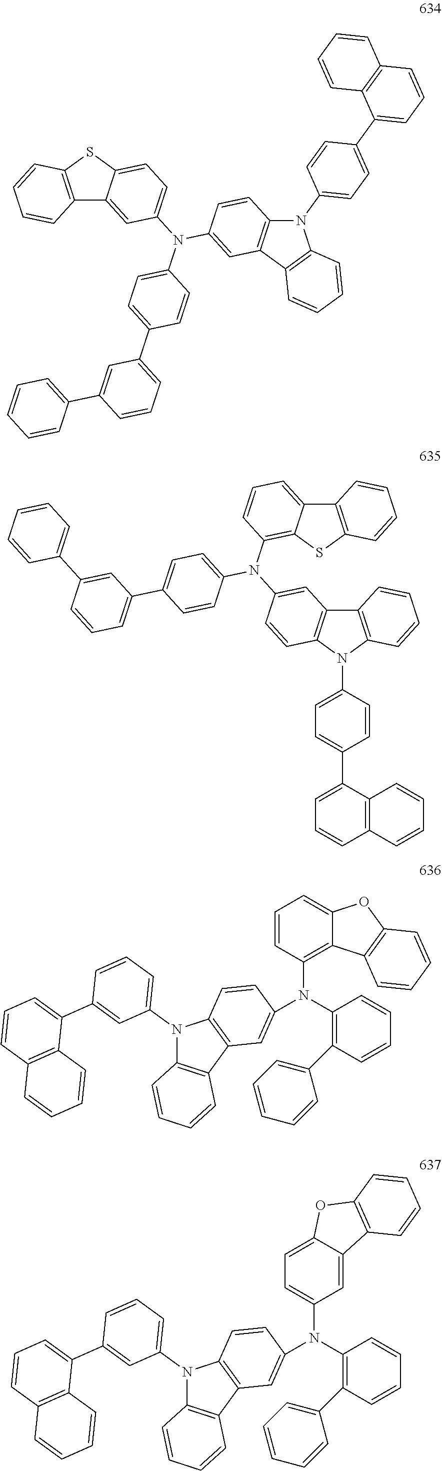 [SMILES: c1ccc(-c2cccc(-c3ccc(N(c4ccc5sc6ccccc6c5c4)c4ccc5c(c4)c4ccccc4n5-c4ccc(-c5cccc6ccccc56)cc4)cc3)c2)cc1 ; c1ccc(-c2cccc(-c3ccc(N(c4ccc5c(c4)c4ccccc4n5-c4ccc(-c5cccc6ccccc56)cc4)c4cccc5c4sc4ccccc45)cc3)c2)cc1 ; c1ccc(-c2ccccc2N(c2ccc3c(c2)c2ccccc2n3-c2cccc(-c3cccc4ccccc34)c2)c2cccc3oc4ccccc4c23)cc1 ; c1ccc(-c2ccccc2N(c2ccc3oc4ccccc4c3c2)c2ccc3c(c2)c2ccccc2n3-c2cccc(-c3cccc4ccccc34)c2)cc1] 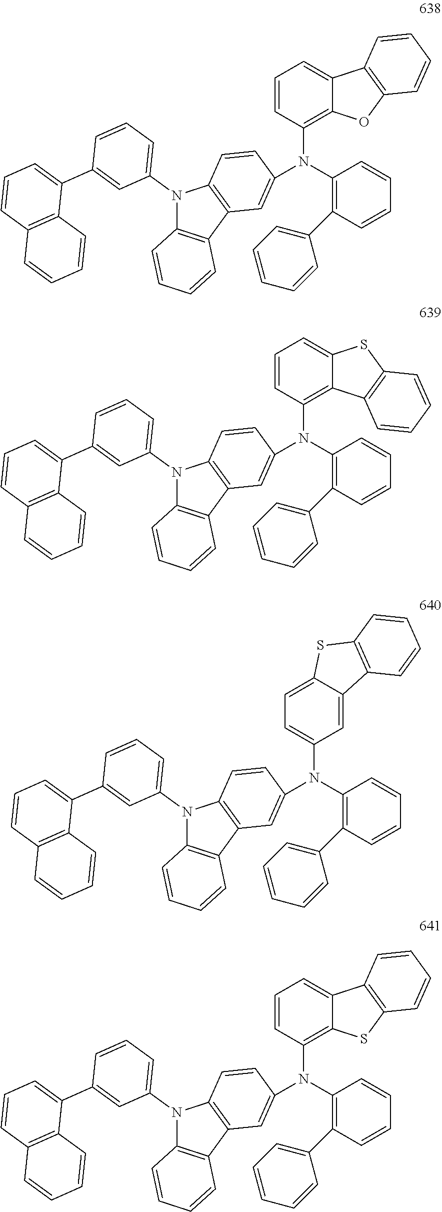 [SMILES: c1ccc(-c2ccccc2N(c2ccc3c(c2)c2ccccc2n3-c2cccc(-c3cccc4ccccc34)c2)c2cccc3c2oc2ccccc23)cc1 ; c1ccc(-c2ccccc2N(c2ccc3c(c2)c2ccccc2n3-c2cccc(-c3cccc4ccccc34)c2)c2cccc3sc4ccccc4c23)cc1 ; c1ccc(-c2ccccc2N(c2ccc3sc4ccccc4c3c2)c2ccc3c(c2)c2ccccc2n3-c2cccc(-c3cccc4ccccc34)c2)cc1 ; c1ccc(-c2ccccc2N(c2ccc3c(c2)c2ccccc2n3-c2cccc(-c3cccc4ccccc34)c2)c2cccc3c2sc2ccccc23)cc1] 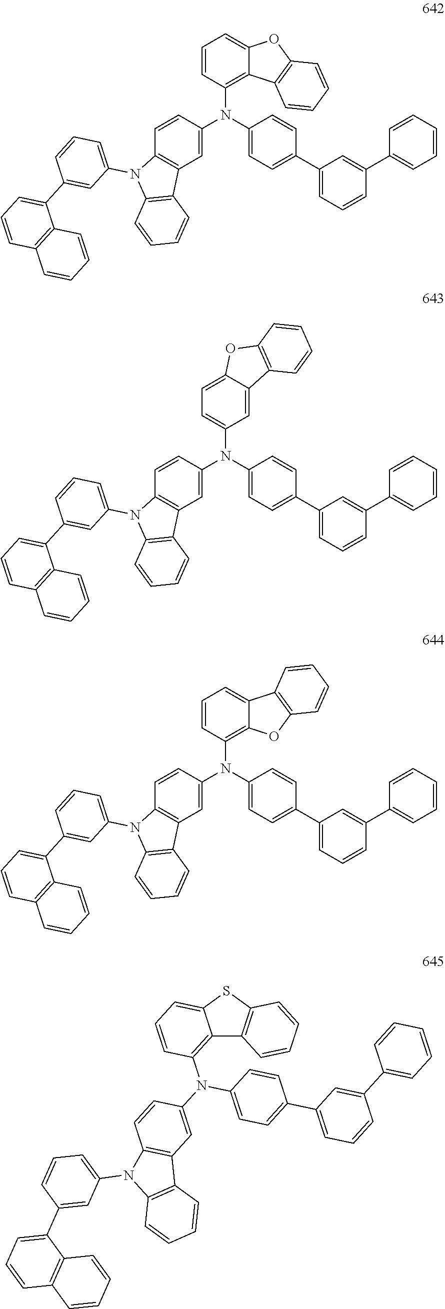 [SMILES: c1ccc(-c2cccc(-c3ccc(N(c4ccc5c(c4)c4ccccc4n5-c4cccc(-c5cccc6ccccc56)c4)c4cccc5oc6ccccc6c45)cc3)c2)cc1 ; c1ccc(-c2cccc(-c3ccc(N(c4ccc5oc6ccccc6c5c4)c4ccc5c(c4)c4ccccc4n5-c4cccc(-c5cccc6ccccc56)c4)cc3)c2)cc1 ; c1ccc(-c2cccc(-c3ccc(N(c4ccc5c(c4)c4ccccc4n5-c4cccc(-c5cccc6ccccc56)c4)c4cccc5c4oc4ccccc45)cc3)c2)cc1 ; c1ccc(-c2cccc(-c3ccc(N(c4ccc5c(c4)c4ccccc4n5-c4cccc(-c5cccc6ccccc56)c4)c4cccc5sc6ccccc6c45)cc3)c2)cc1] 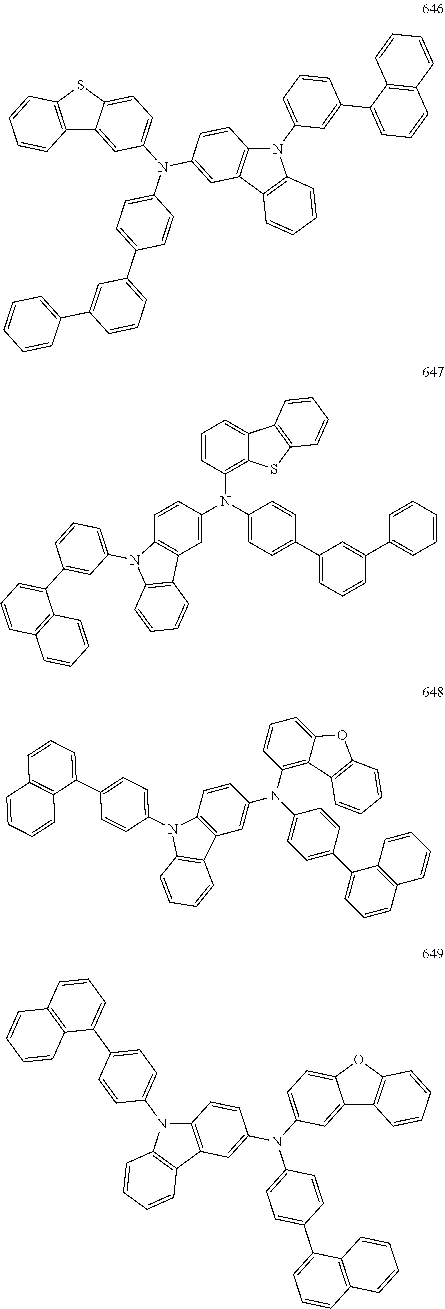 [SMILES: c1ccc(-c2cccc(-c3ccc(N(c4ccc5sc6ccccc6c5c4)c4ccc5c(c4)c4ccccc4n5-c4cccc(-c5cccc6ccccc56)c4)cc3)c2)cc1 ; c1ccc(-c2cccc(-c3ccc(N(c4ccc5c(c4)c4ccccc4n5-c4cccc(-c5cccc6ccccc56)c4)c4cccc5c4sc4ccccc45)cc3)c2)cc1 ; c1ccc2c(-c3ccc(N(c4ccc5c(c4)c4ccccc4n5-c4ccc(-c5cccc6ccccc56)cc4)c4cccc5oc6ccccc6c45)cc3)cccc2c1 ; c1ccc2c(-c3ccc(N(c4ccc5oc6ccccc6c5c4)c4ccc5c(c4)c4ccccc4n5-c4ccc(-c5cccc6ccccc56)cc4)cc3)cccc2c1] 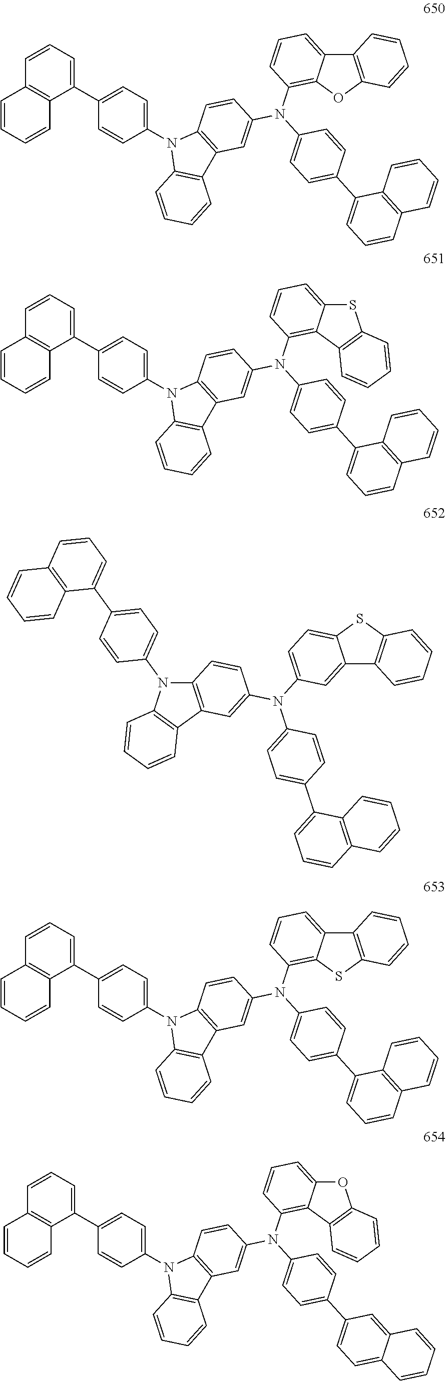 [SMILES: c1ccc2c(-c3ccc(N(c4ccc5c(c4)c4ccccc4n5-c4ccc(-c5cccc6ccccc56)cc4)c4cccc5c4oc4ccccc45)cc3)cccc2c1 ; c1ccc2c(-c3ccc(N(c4ccc5c(c4)c4ccccc4n5-c4ccc(-c5cccc6ccccc56)cc4)c4cccc5sc6ccccc6c45)cc3)cccc2c1 ; c1ccc2c(-c3ccc(N(c4ccc5sc6ccccc6c5c4)c4ccc5c(c4)c4ccccc4n5-c4ccc(-c5cccc6ccccc56)cc4)cc3)cccc2c1 ; c1ccc2c(-c3ccc(N(c4ccc5c(c4)c4ccccc4n5-c4ccc(-c5cccc6ccccc56)cc4)c4cccc5c4sc4ccccc45)cc3)cccc2c1 ; c1ccc2cc(-c3ccc(N(c4ccc5c(c4)c4ccccc4n5-c4ccc(-c5cccc6ccccc56)cc4)c4cccc5oc6ccccc6c45)cc3)ccc2c1] 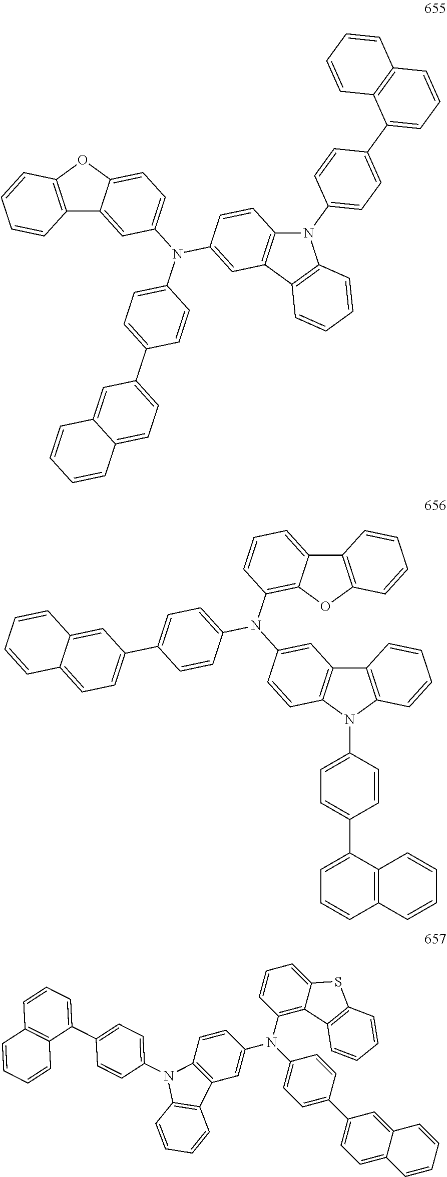 [SMILES: c1ccc2cc(-c3ccc(N(c4ccc5oc6ccccc6c5c4)c4ccc5c(c4)c4ccccc4n5-c4ccc(-c5cccc6ccccc56)cc4)cc3)ccc2c1 ; c1ccc2cc(-c3ccc(N(c4ccc5c(c4)c4ccccc4n5-c4ccc(-c5cccc6ccccc56)cc4)c4cccc5c4oc4ccccc45)cc3)ccc2c1 ; c1ccc2cc(-c3ccc(N(c4ccc5c(c4)c4ccccc4n5-c4ccc(-c5cccc6ccccc56)cc4)c4cccc5sc6ccccc6c45)cc3)ccc2c1] 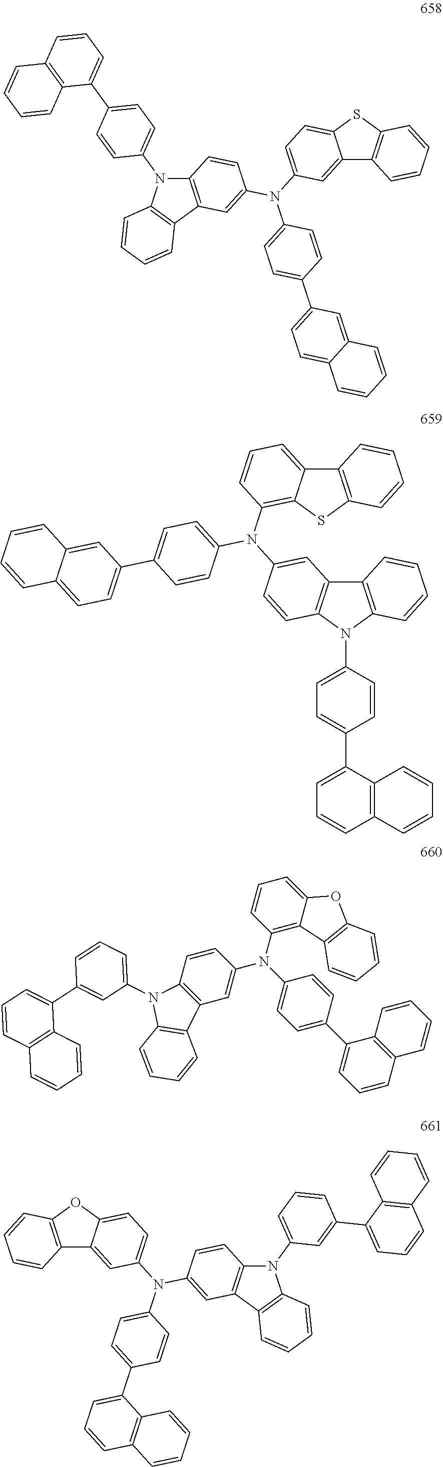 [SMILES: c1ccc2cc(-c3ccc(N(c4ccc5sc6ccccc6c5c4)c4ccc5c(c4)c4ccccc4n5-c4ccc(-c5cccc6ccccc56)cc4)cc3)ccc2c1 ; c1ccc2cc(-c3ccc(N(c4ccc5c(c4)c4ccccc4n5-c4ccc(-c5cccc6ccccc56)cc4)c4cccc5c4sc4ccccc45)cc3)ccc2c1 ; c1cc(-c2cccc3ccccc23)cc(-n2c3ccccc3c3cc(N(c4ccc(-c5cccc6ccccc56)cc4)c4cccc5oc6ccccc6c45)ccc32)c1 ; c1cc(-c2cccc3ccccc23)cc(-n2c3ccccc3c3cc(N(c4ccc(-c5cccc6ccccc56)cc4)c4ccc5oc6ccccc6c5c4)ccc32)c1] 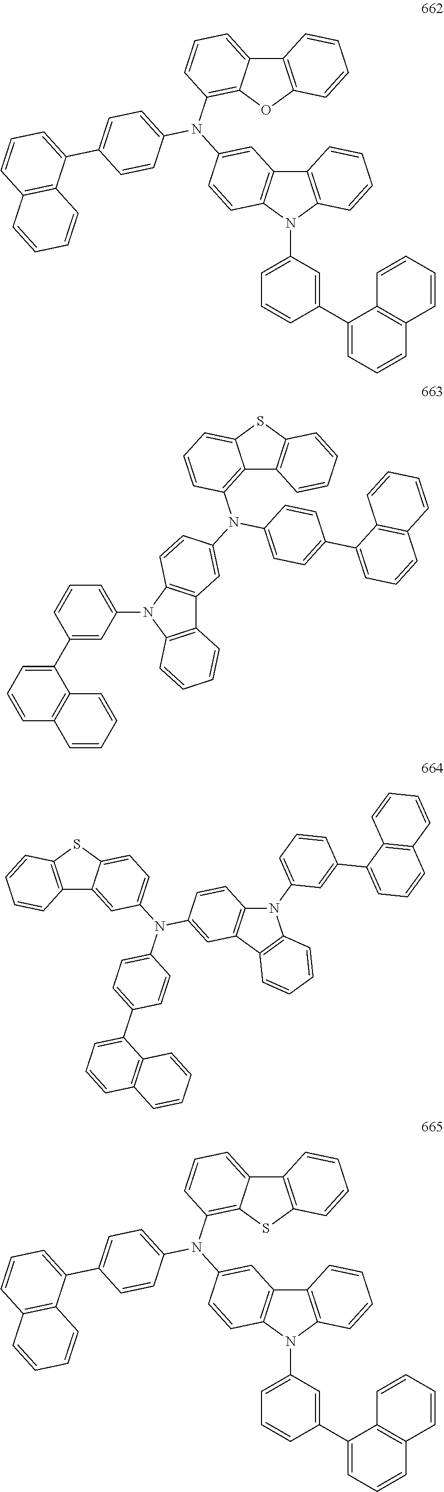 [SMILES: c1cc(-c2cccc3ccccc23)cc(-n2c3ccccc3c3cc(N(c4ccc(-c5cccc6ccccc56)cc4)c4cccc5c4oc4ccccc45)ccc32)c1 ; c1cc(-c2cccc3ccccc23)cc(-n2c3ccccc3c3cc(N(c4ccc(-c5cccc6ccccc56)cc4)c4cccc5sc6ccccc6c45)ccc32)c1 ; c1cc(-c2cccc3ccccc23)cc(-n2c3ccccc3c3cc(N(c4ccc(-c5cccc6ccccc56)cc4)c4ccc5sc6ccccc6c5c4)ccc32)c1 ; c1cc(-c2cccc3ccccc23)cc(-n2c3ccccc3c3cc(N(c4ccc(-c5cccc6ccccc56)cc4)c4cccc5c4sc4ccccc45)ccc32)c1] 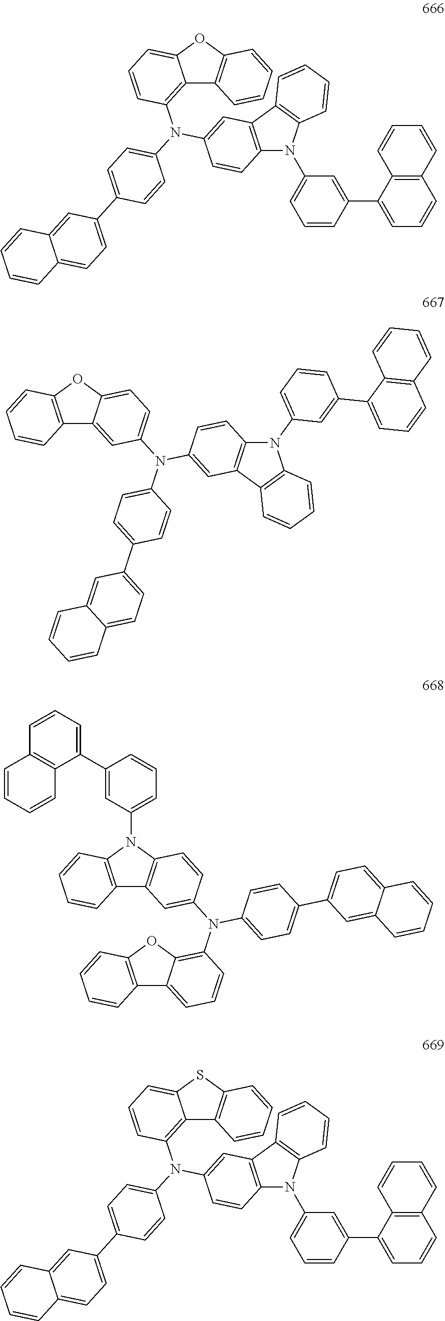 [SMILES: c1cc(-c2cccc3ccccc23)cc(-n2c3ccccc3c3cc(N(c4ccc(-c5ccc6ccccc6c5)cc4)c4cccc5oc6ccccc6c45)ccc32)c1 ; c1cc(-c2cccc3ccccc23)cc(-n2c3ccccc3c3cc(N(c4ccc(-c5ccc6ccccc6c5)cc4)c4ccc5oc6ccccc6c5c4)ccc32)c1 ; c1cc(-c2cccc3ccccc23)cc(-n2c3ccccc3c3cc(N(c4ccc(-c5ccc6ccccc6c5)cc4)c4cccc5c4oc4ccccc45)ccc32)c1 ; c1cc(-c2cccc3ccccc23)cc(-n2c3ccccc3c3cc(N(c4ccc(-c5ccc6ccccc6c5)cc4)c4cccc5sc6ccccc6c45)ccc32)c1] 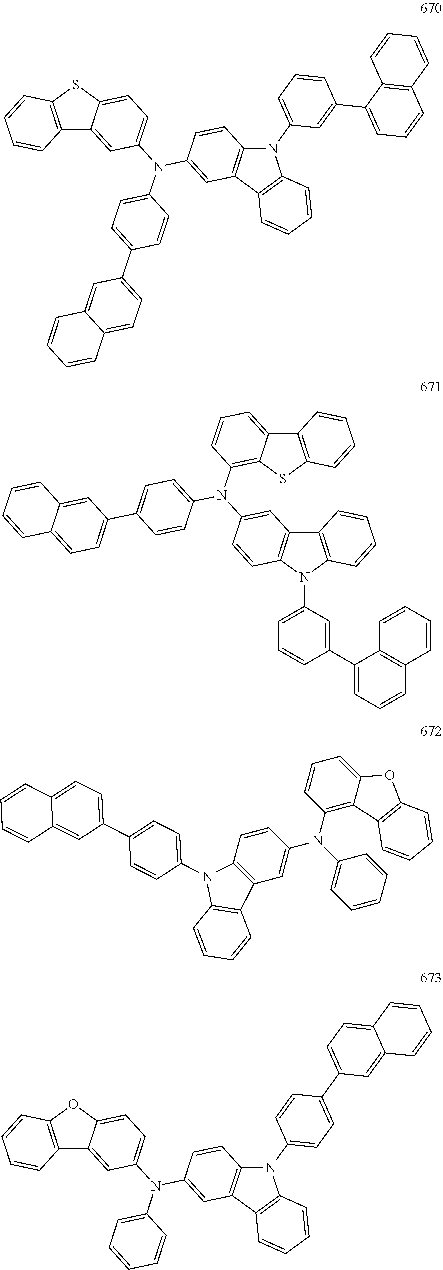 [SMILES: c1cc(-c2cccc3ccccc23)cc(-n2c3ccccc3c3cc(N(c4ccc(-c5ccc6ccccc6c5)cc4)c4ccc5sc6ccccc6c5c4)ccc32)c1 ; c1cc(-c2cccc3ccccc23)cc(-n2c3ccccc3c3cc(N(c4ccc(-c5ccc6ccccc6c5)cc4)c4cccc5c4sc4ccccc45)ccc32)c1 ; c1ccc(N(c2ccc3c(c2)c2ccccc2n3-c2ccc(-c3ccc4ccccc4c3)cc2)c2cccc3oc4ccccc4c23)cc1 ; c1ccc(N(c2ccc3oc4ccccc4c3c2)c2ccc3c(c2)c2ccccc2n3-c2ccc(-c3ccc4ccccc4c3)cc2)cc1] 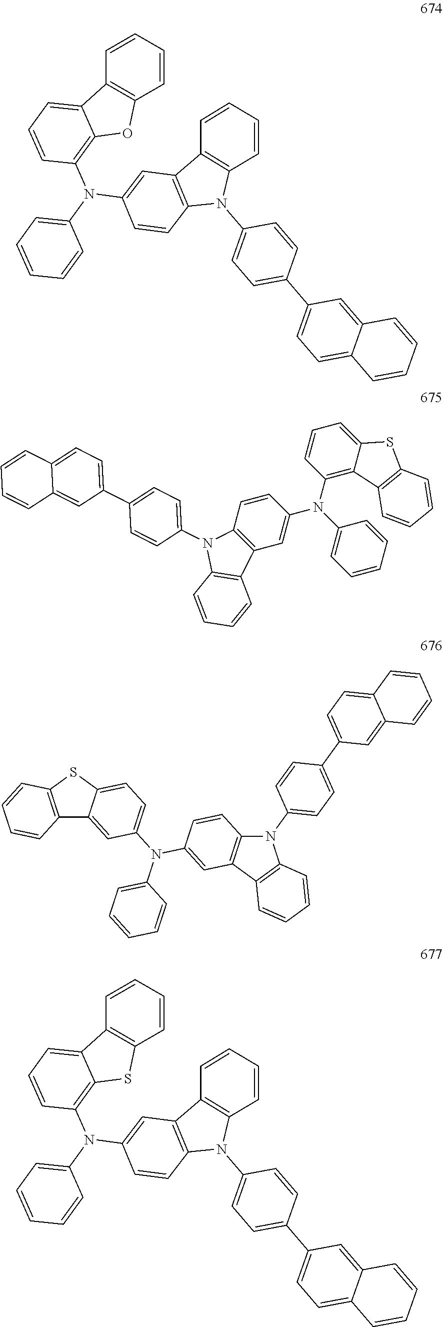 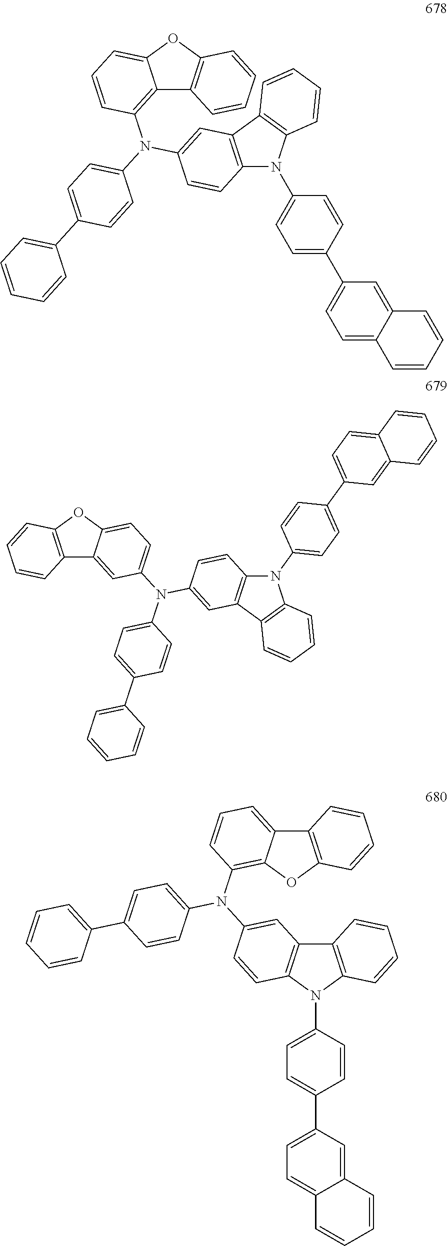 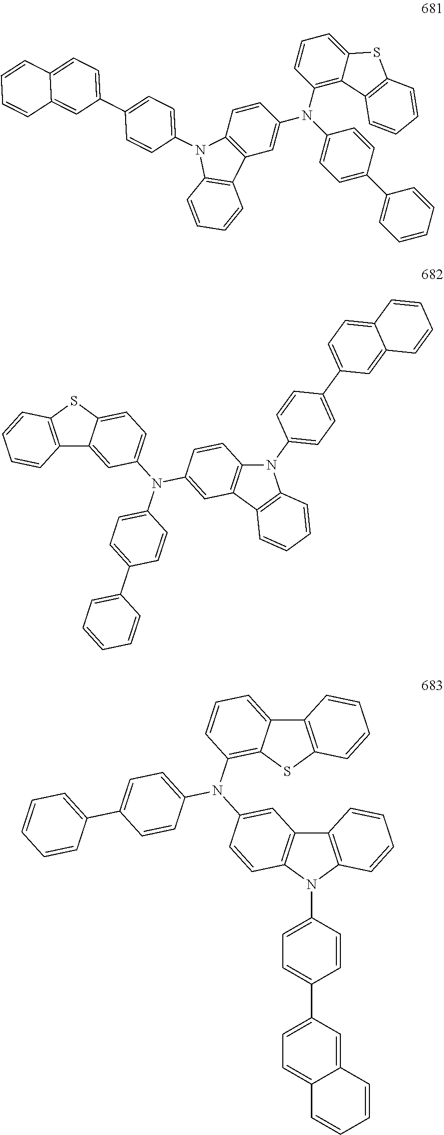 [SMILES: c1ccc(-c2ccc(N(c3ccc4c(c3)c3ccccc3n4-c3ccc(-c4ccc5ccccc5c4)cc3)c3cccc4sc5ccccc5c34)cc2)cc1 ; c1ccc(-c2ccc(N(c3ccc4sc5ccccc5c4c3)c3ccc4c(c3)c3ccccc3n4-c3ccc(-c4ccc5ccccc5c4)cc3)cc2)cc1 ; c1ccc(-c2ccc(N(c3ccc4c(c3)c3ccccc3n4-c3ccc(-c4ccc5ccccc5c4)cc3)c3cccc4c3sc3ccccc34)cc2)cc1] 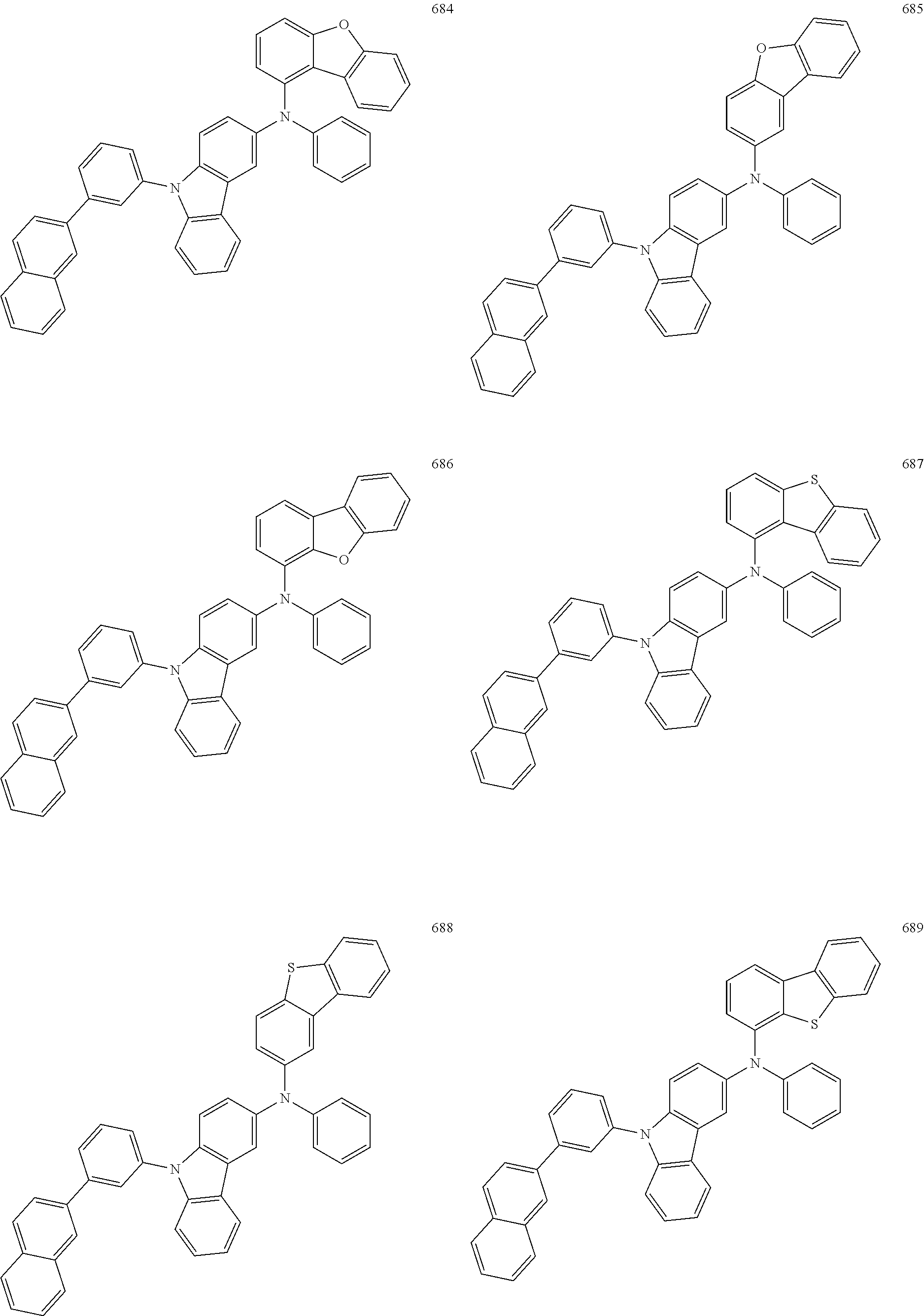 [SMILES: c1ccc(N(c2ccc3c(c2)c2ccccc2n3-c2cccc(-c3ccc4ccccc4c3)c2)c2cccc3oc4ccccc4c23)cc1 ; c1ccc(N(c2ccc3oc4ccccc4c3c2)c2ccc3c(c2)c2ccccc2n3-c2cccc(-c3ccc4ccccc4c3)c2)cc1 ; c1ccc(N(c2ccc3c(c2)c2ccccc2n3-c2cccc(-c3ccc4ccccc4c3)c2)c2cccc3c2oc2ccccc23)cc1 ; c1ccc(N(c2ccc3c(c2)c2ccccc2n3-c2cccc(-c3ccc4ccccc4c3)c2)c2cccc3sc4ccccc4c23)cc1 ; c1ccc(N(c2ccc3sc4ccccc4c3c2)c2ccc3c(c2)c2ccccc2n3-c2cccc(-c3ccc4ccccc4c3)c2)cc1 ; c1ccc(N(c2ccc3c(c2)c2ccccc2n3-c2cccc(-c3ccc4ccccc4c3)c2)c2cccc3c2sc2ccccc23)cc1] 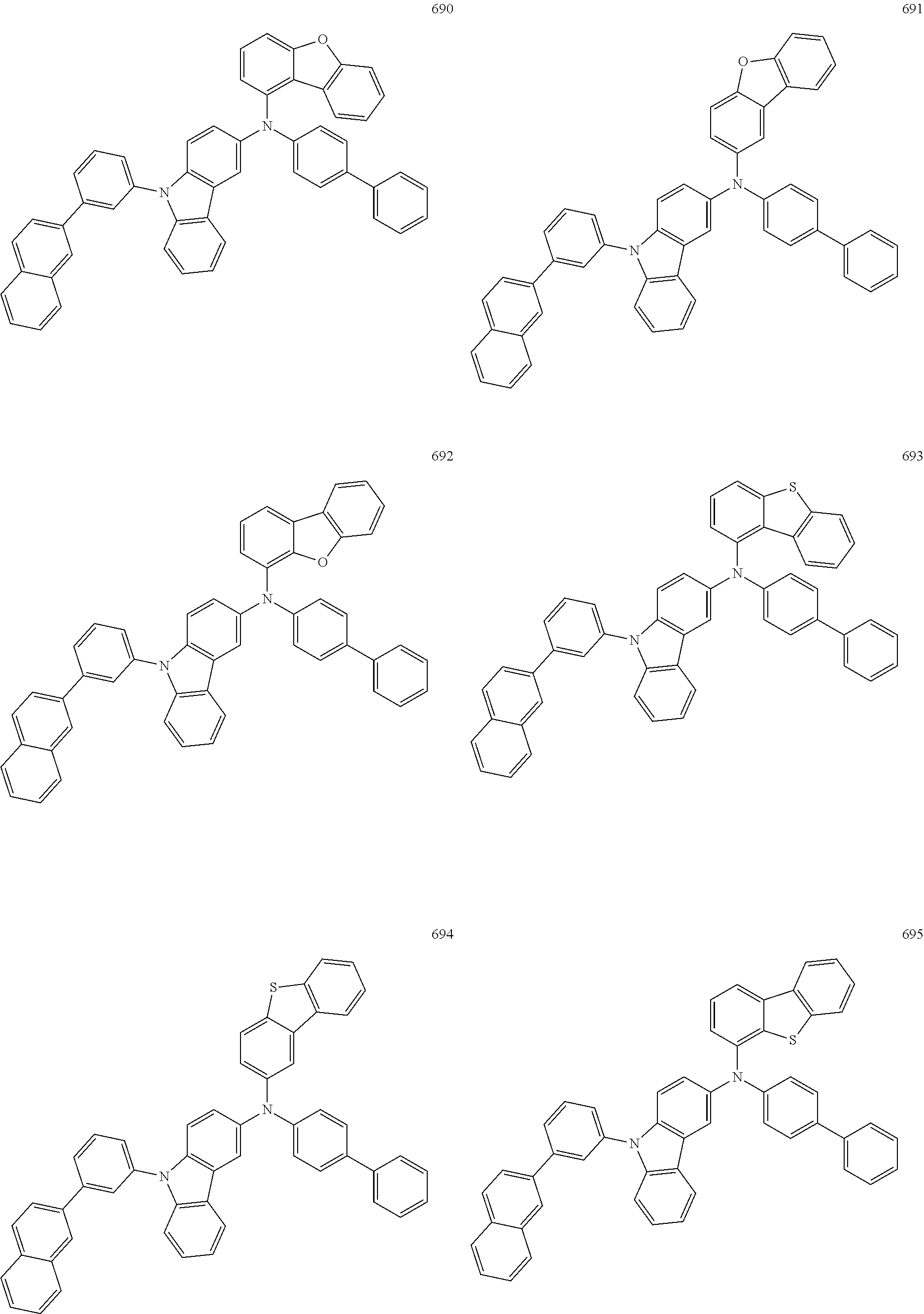 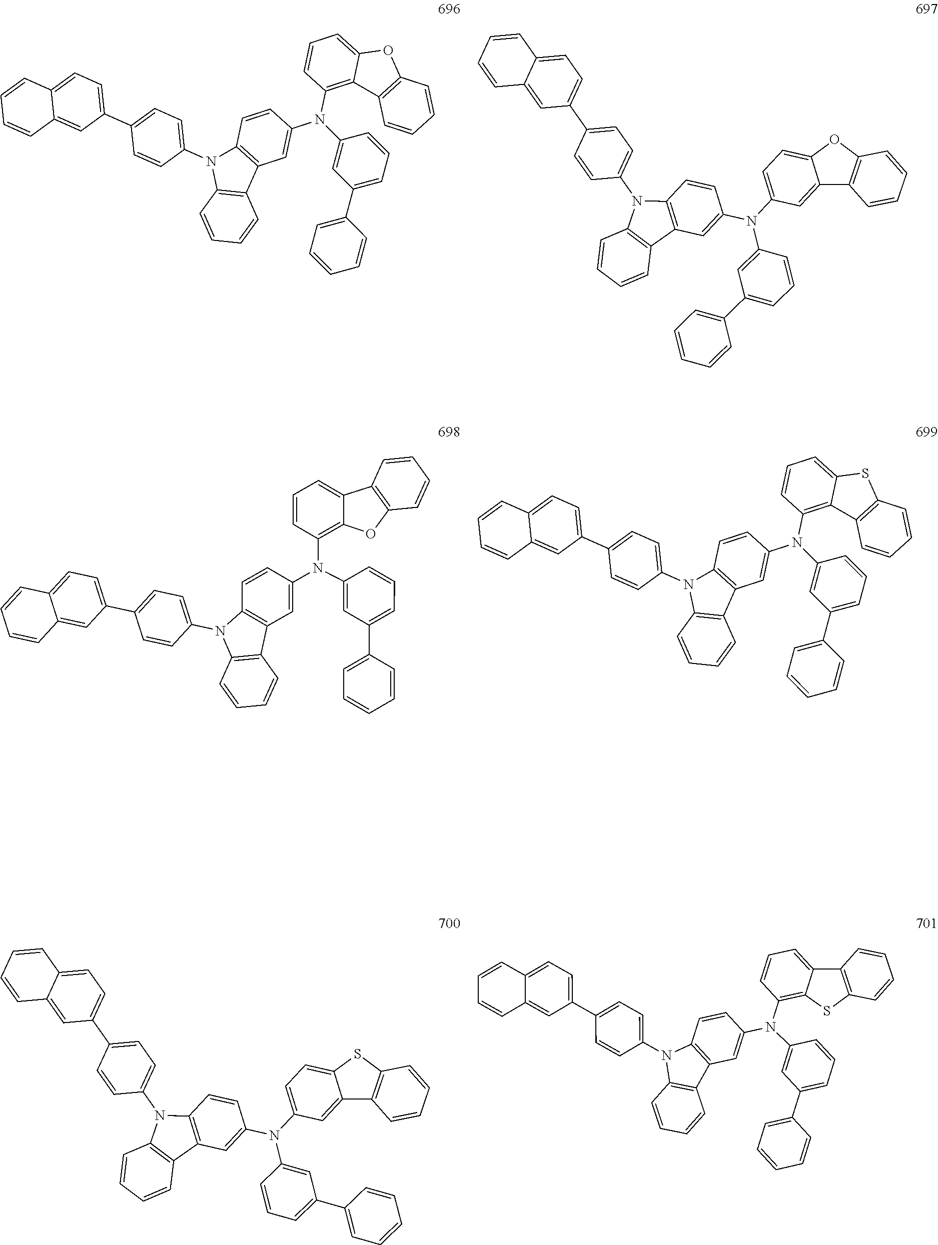 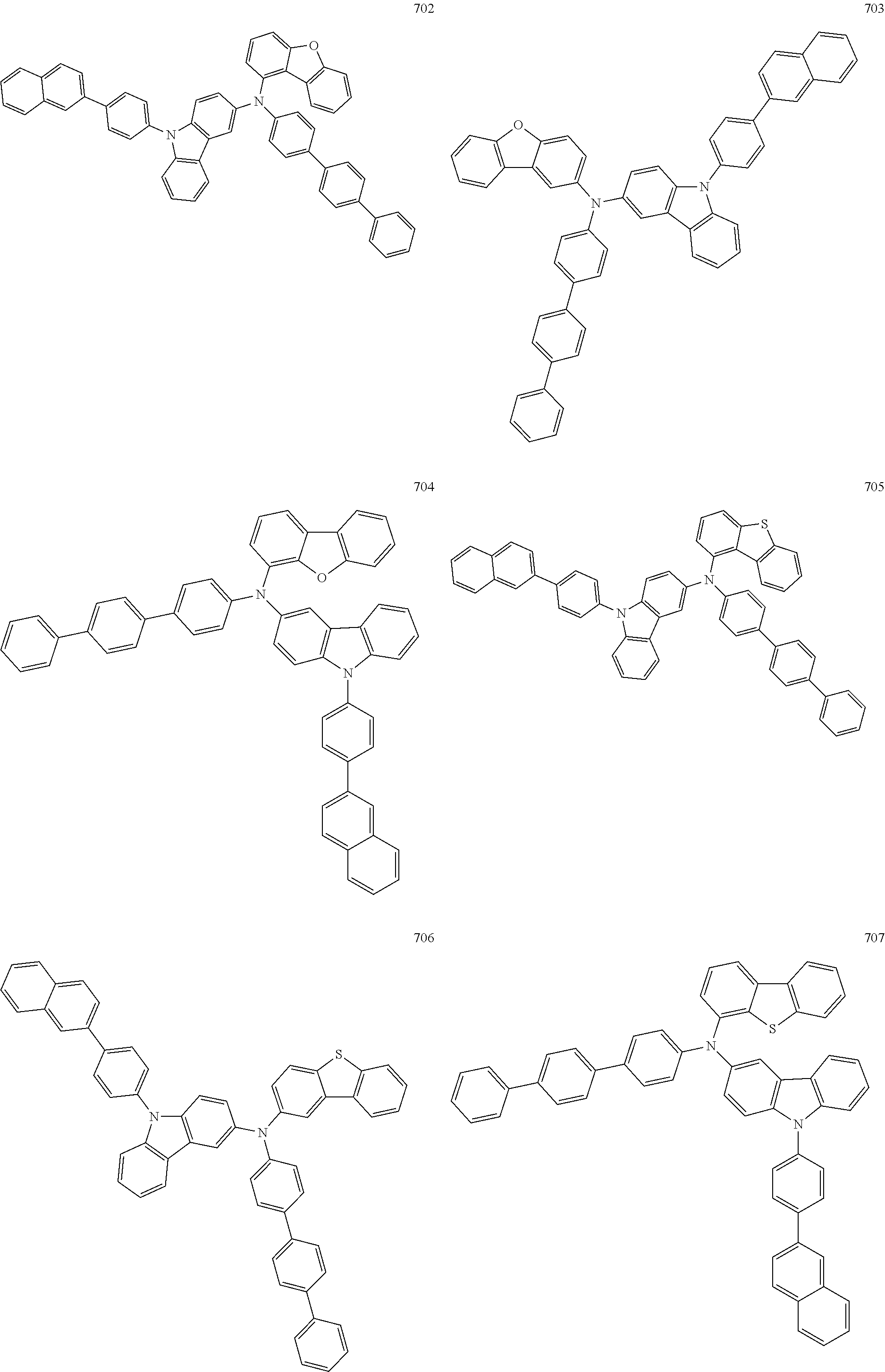 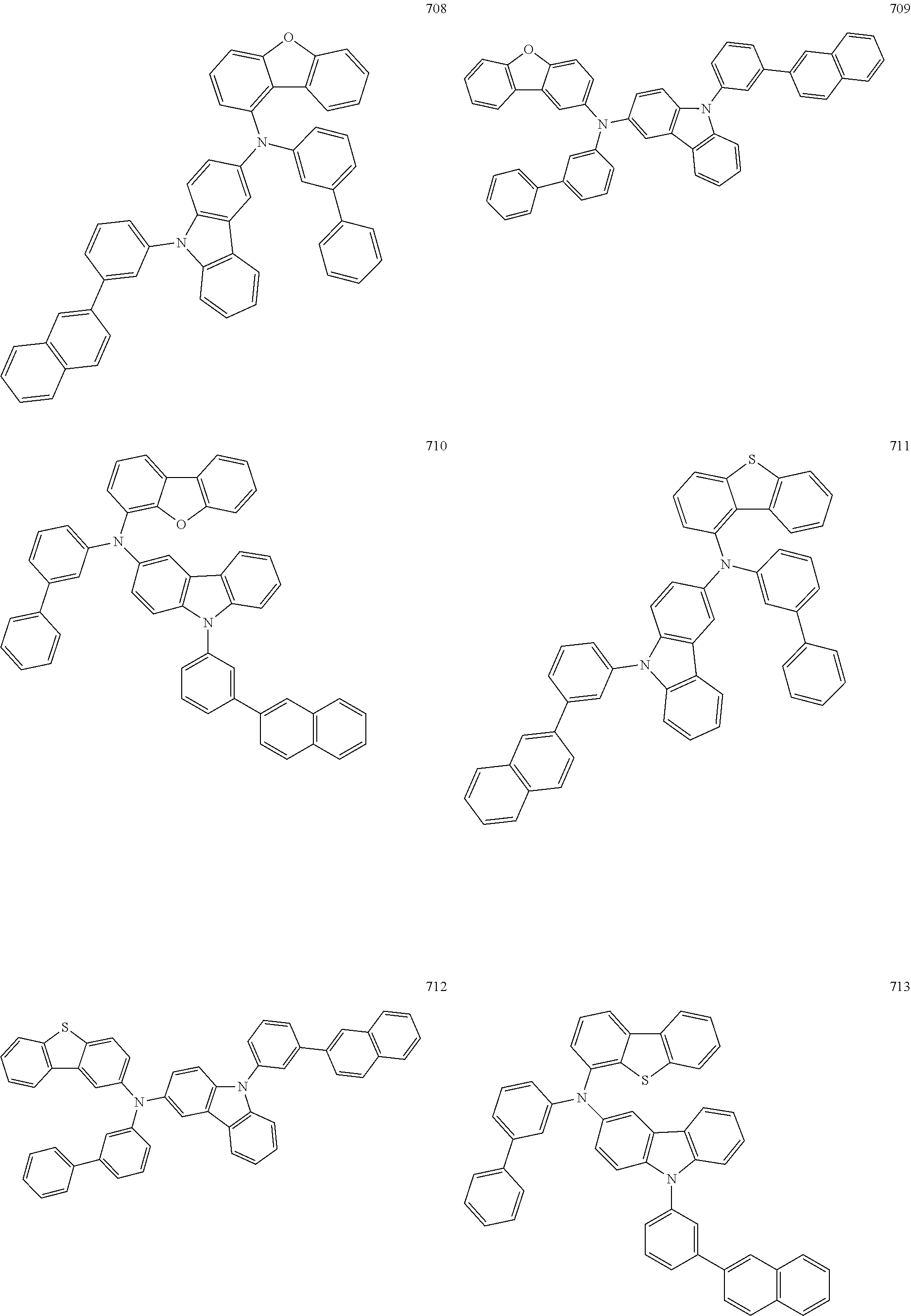 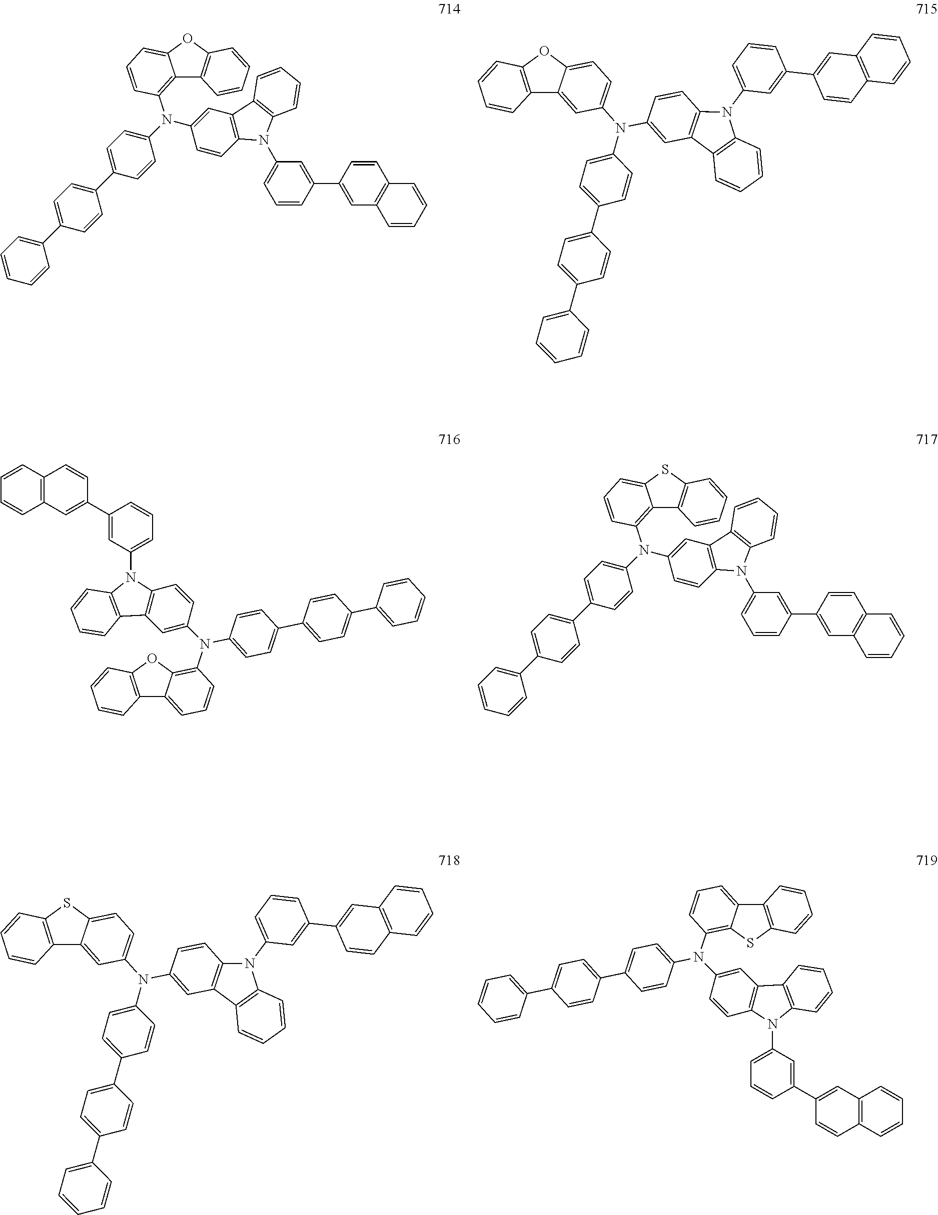 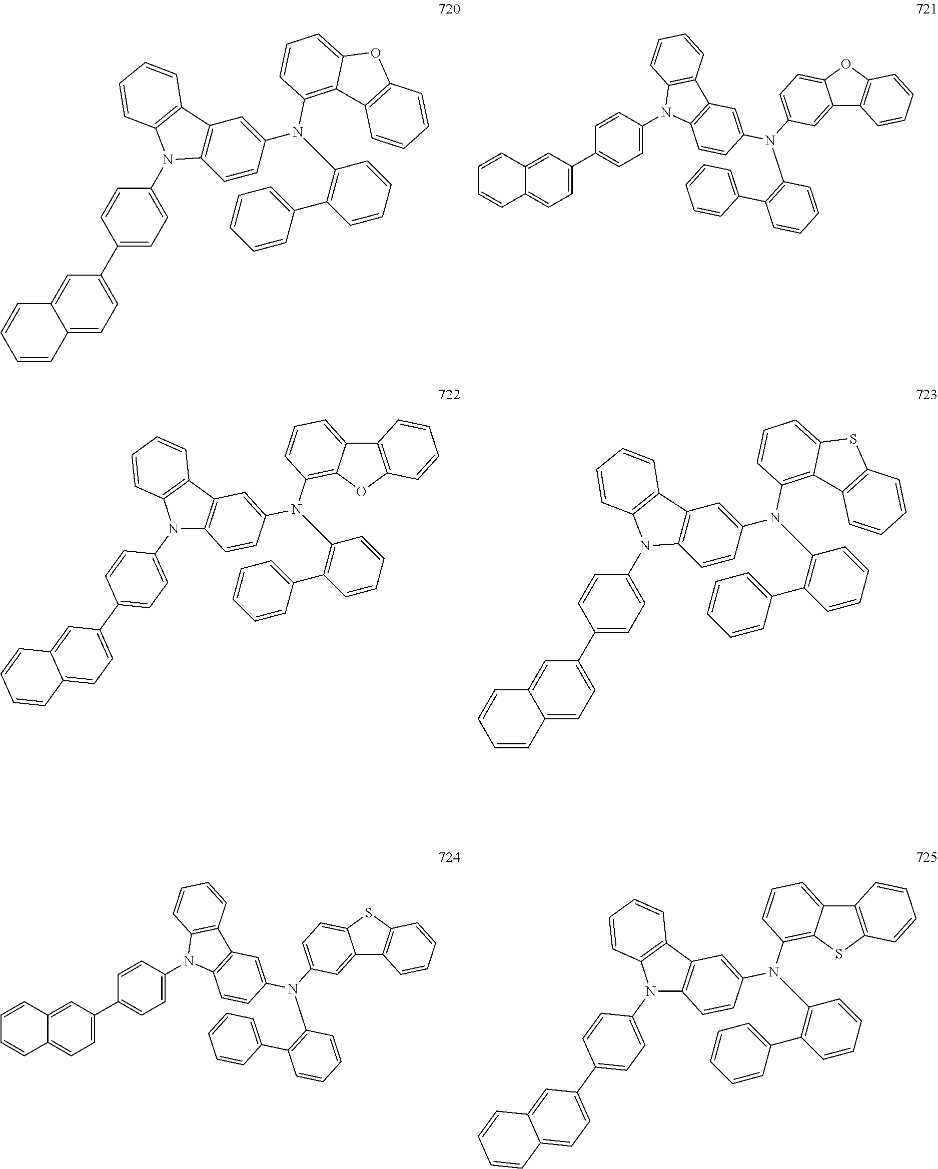 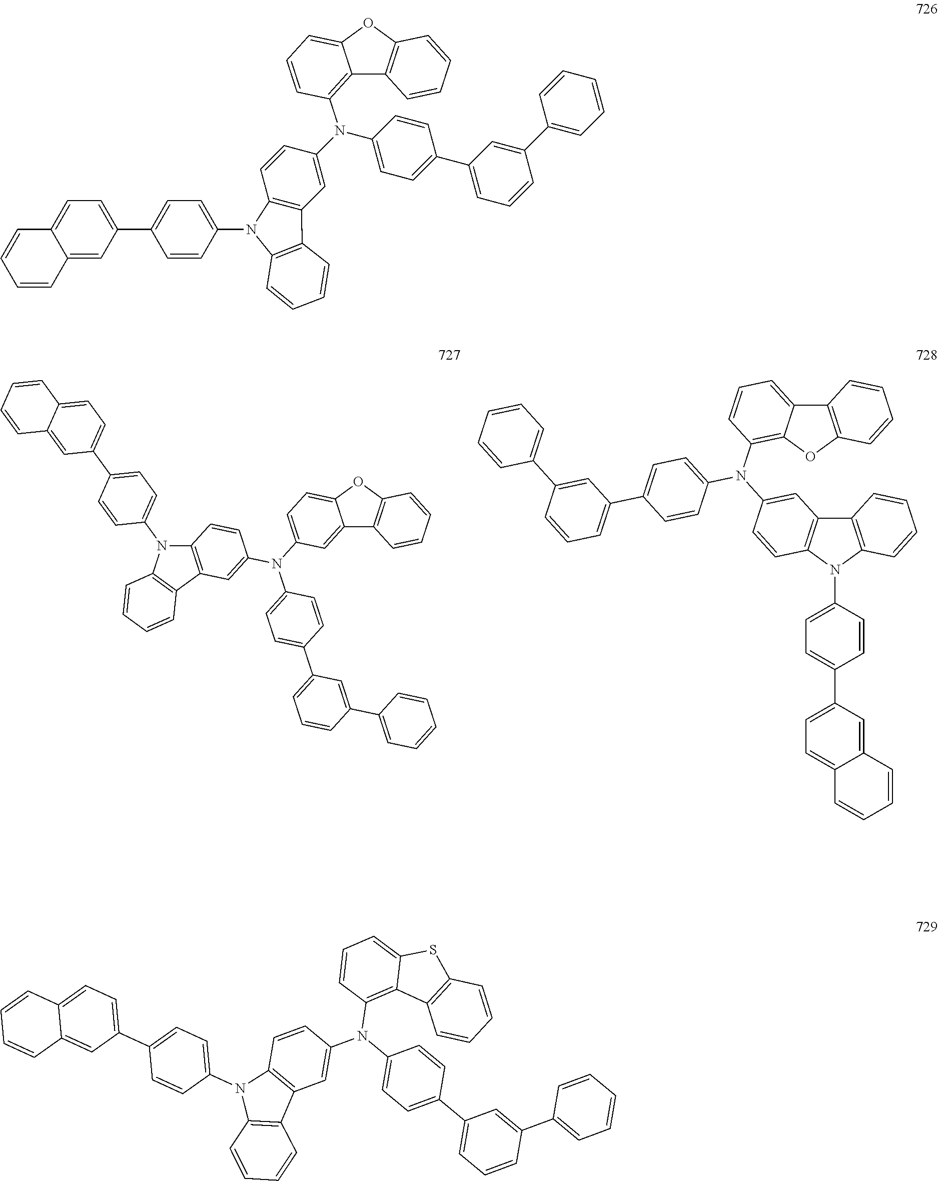 [SMILES: c1ccc(-c2cccc(-c3ccc(N(c4ccc5c(c4)c4ccccc4n5-c4ccc(-c5ccc6ccccc6c5)cc4)c4cccc5oc6ccccc6c45)cc3)c2)cc1 ; c1ccc(-c2cccc(-c3ccc(N(c4ccc5oc6ccccc6c5c4)c4ccc5c(c4)c4ccccc4n5-c4ccc(-c5ccc6ccccc6c5)cc4)cc3)c2)cc1 ; c1ccc(-c2cccc(-c3ccc(N(c4ccc5c(c4)c4ccccc4n5-c4ccc(-c5ccc6ccccc6c5)cc4)c4cccc5c4oc4ccccc45)cc3)c2)cc1 ; c1ccc(-c2cccc(-c3ccc(N(c4ccc5c(c4)c4ccccc4n5-c4ccc(-c5ccc6ccccc6c5)cc4)c4cccc5sc6ccccc6c45)cc3)c2)cc1] 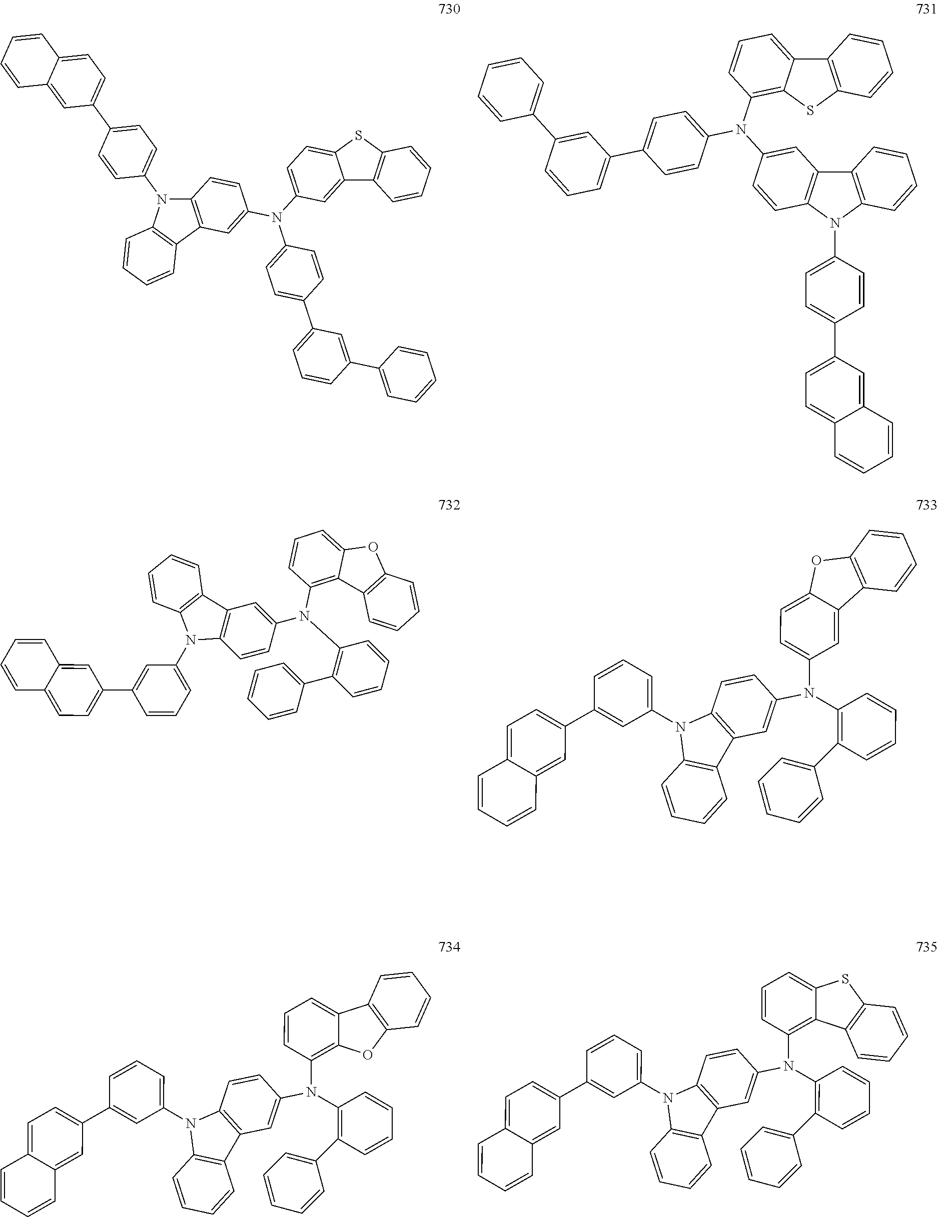 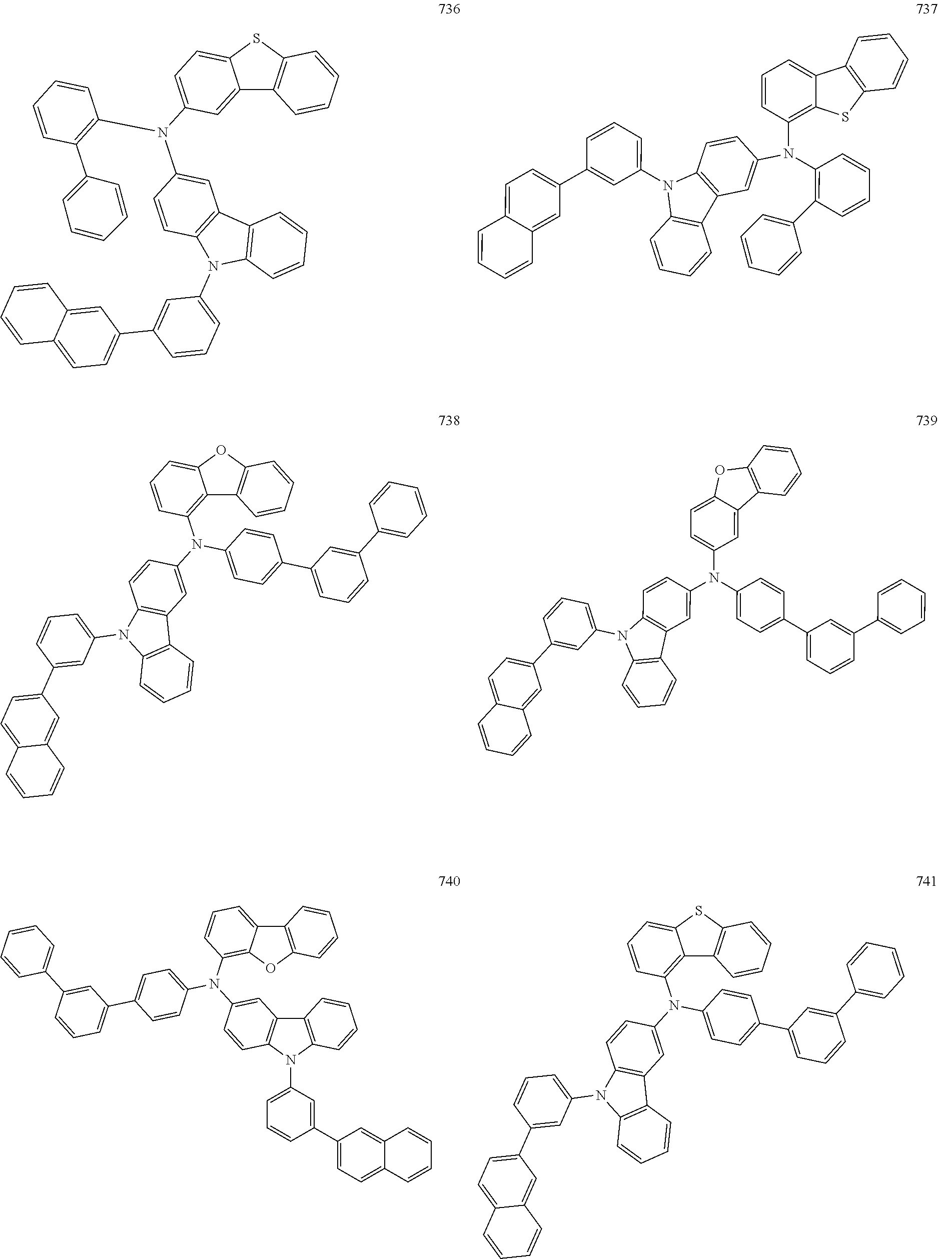 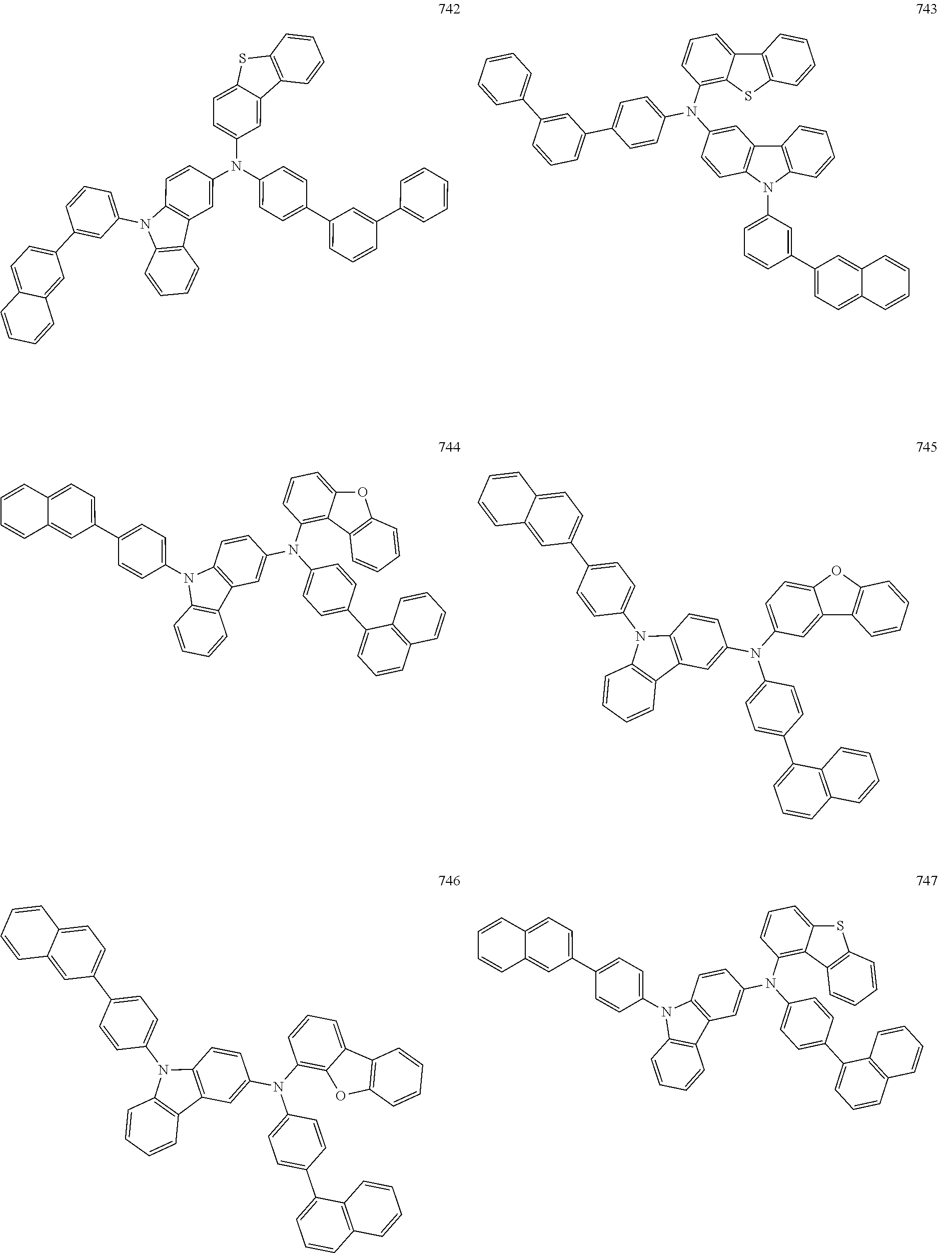 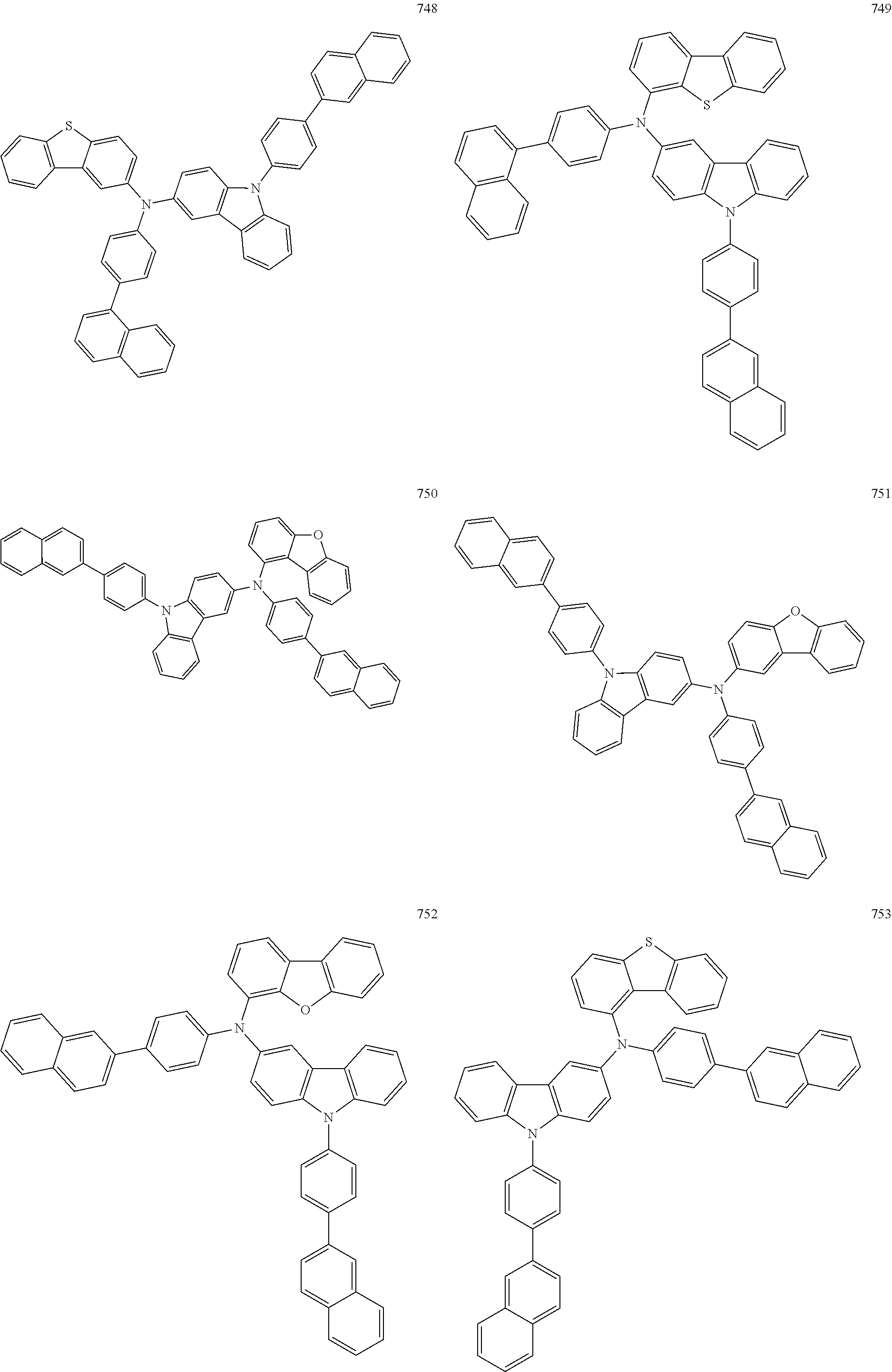 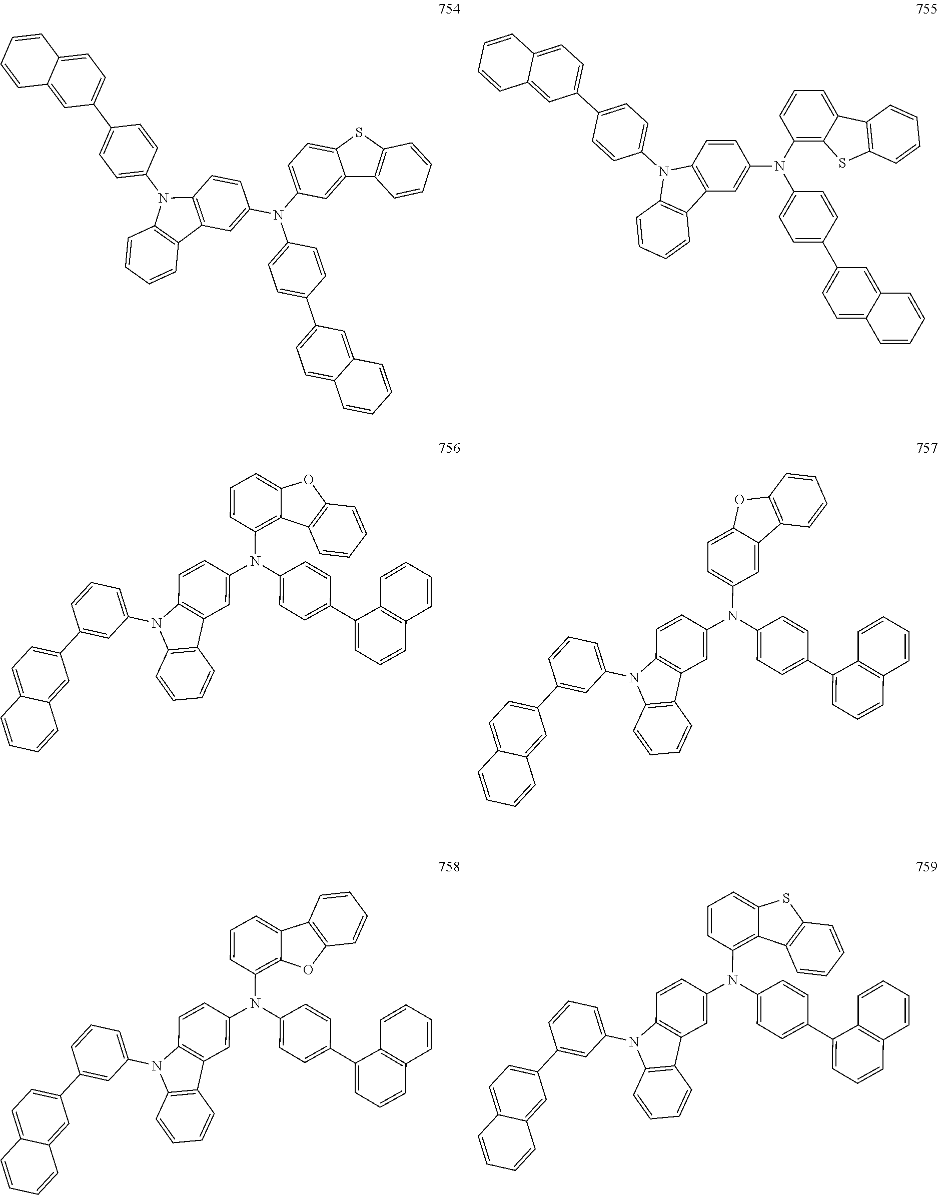 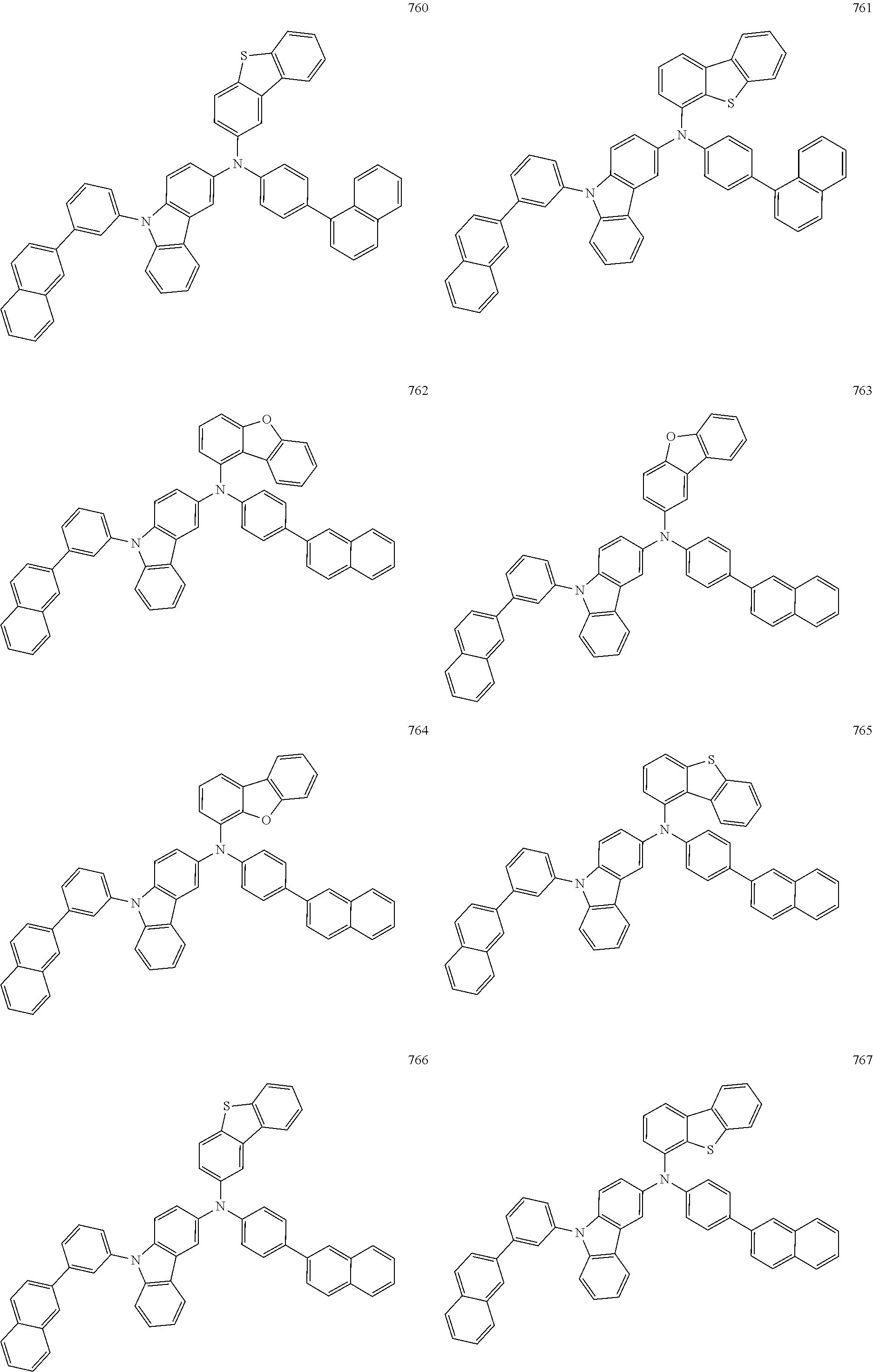 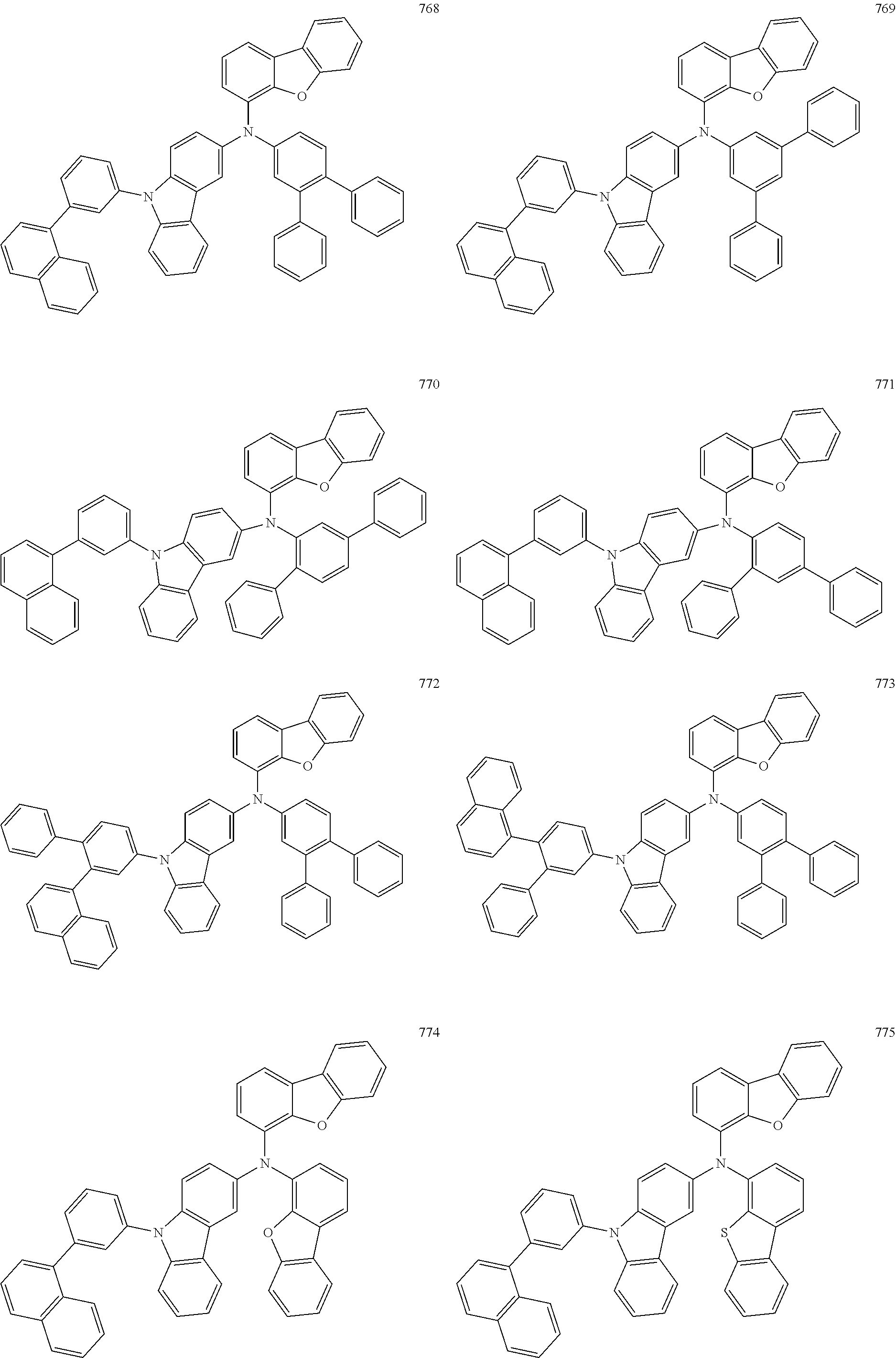 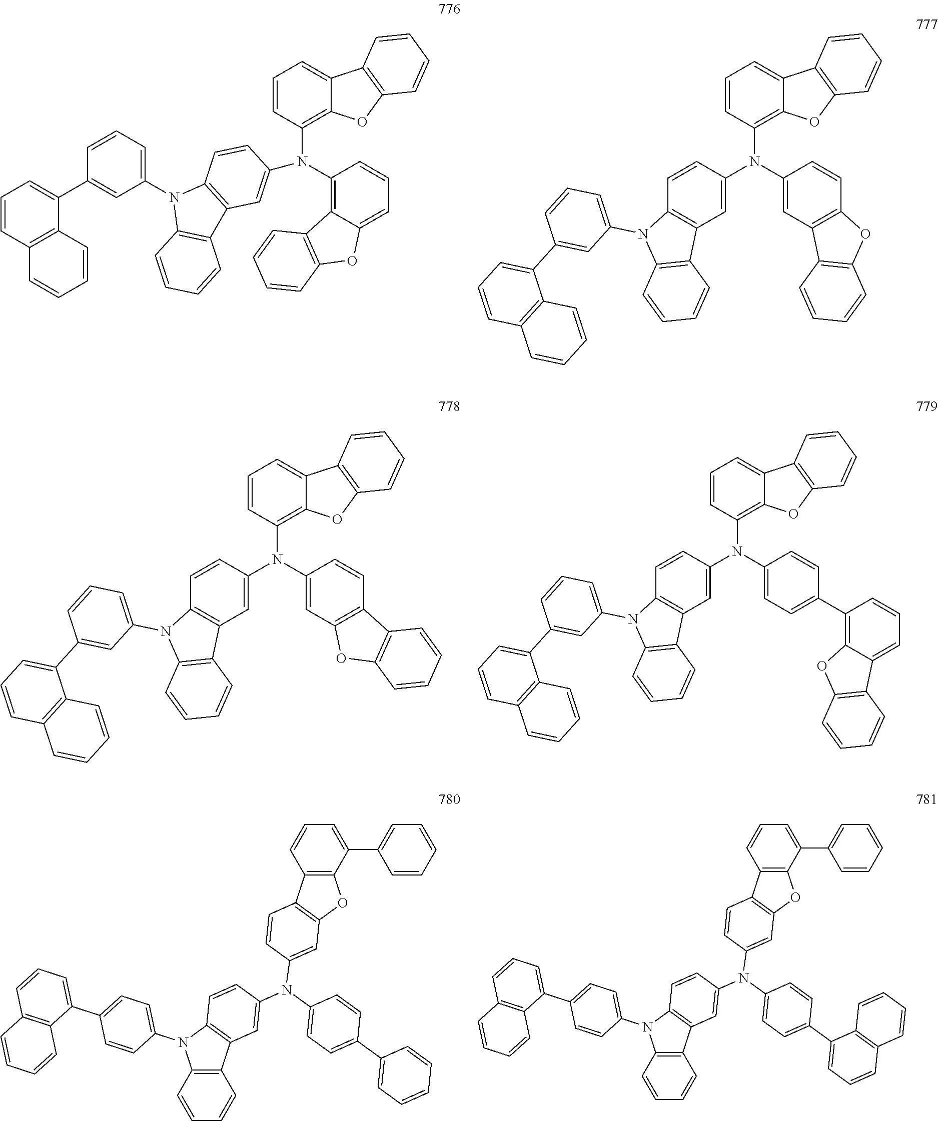 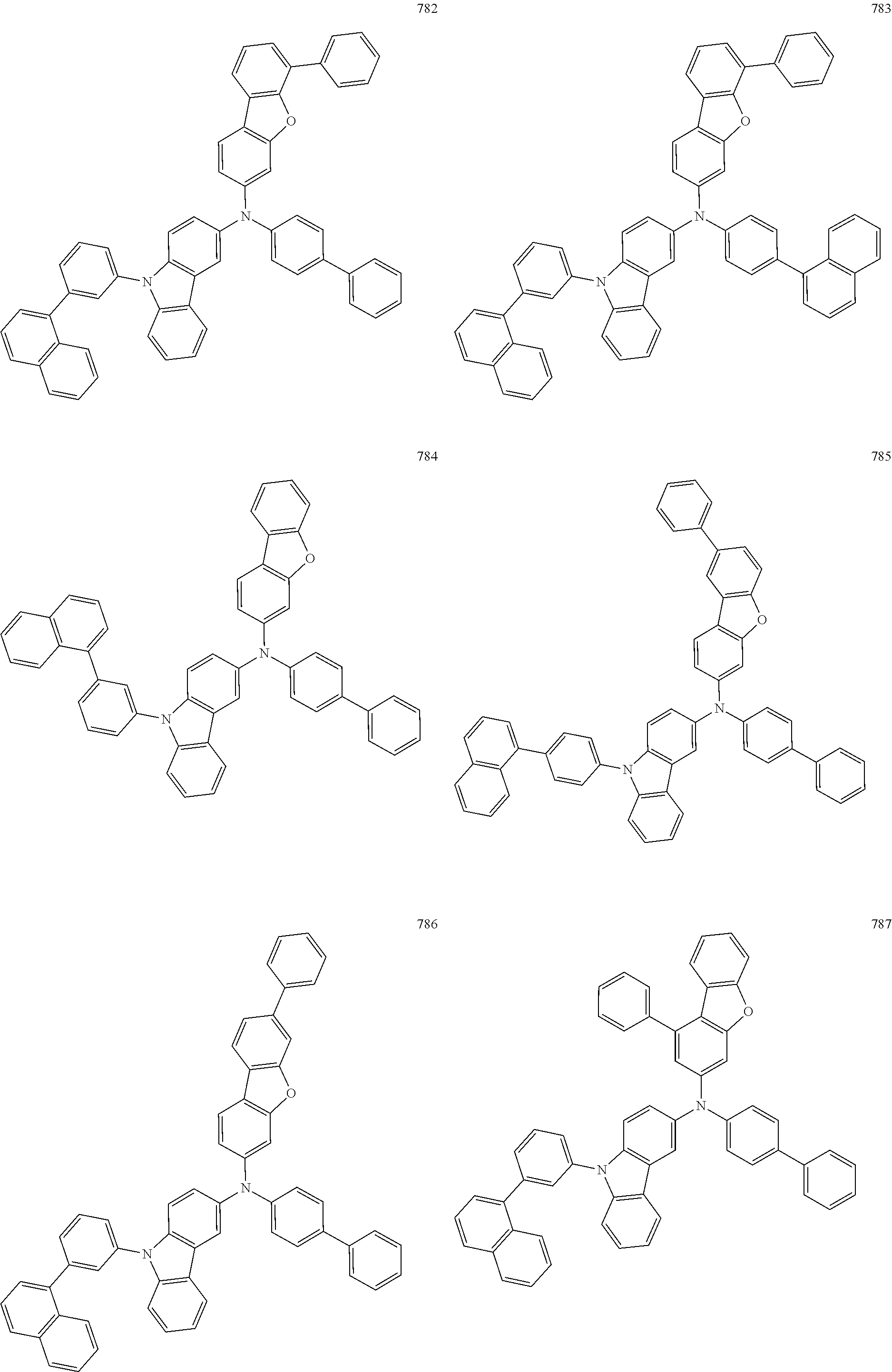 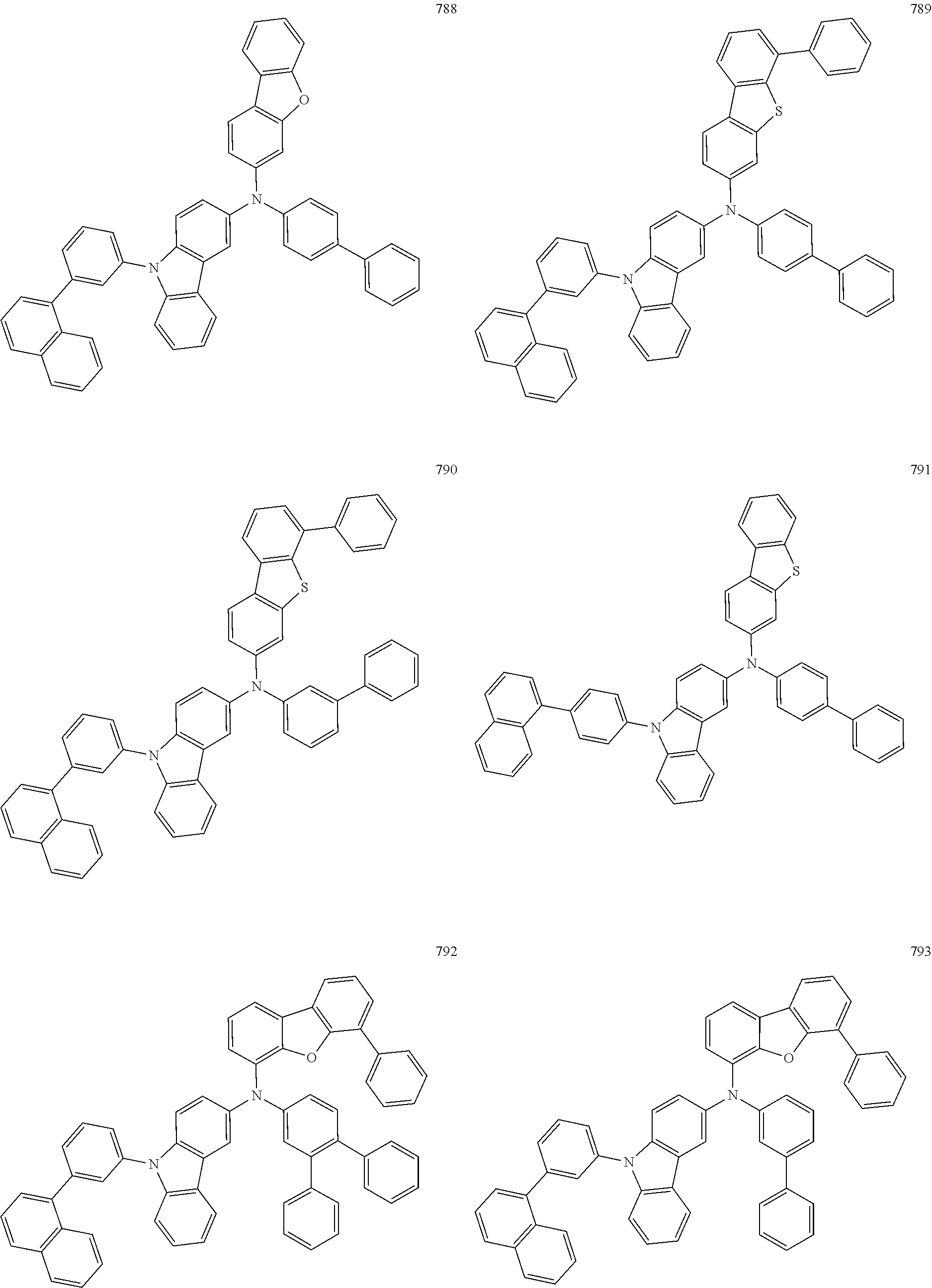 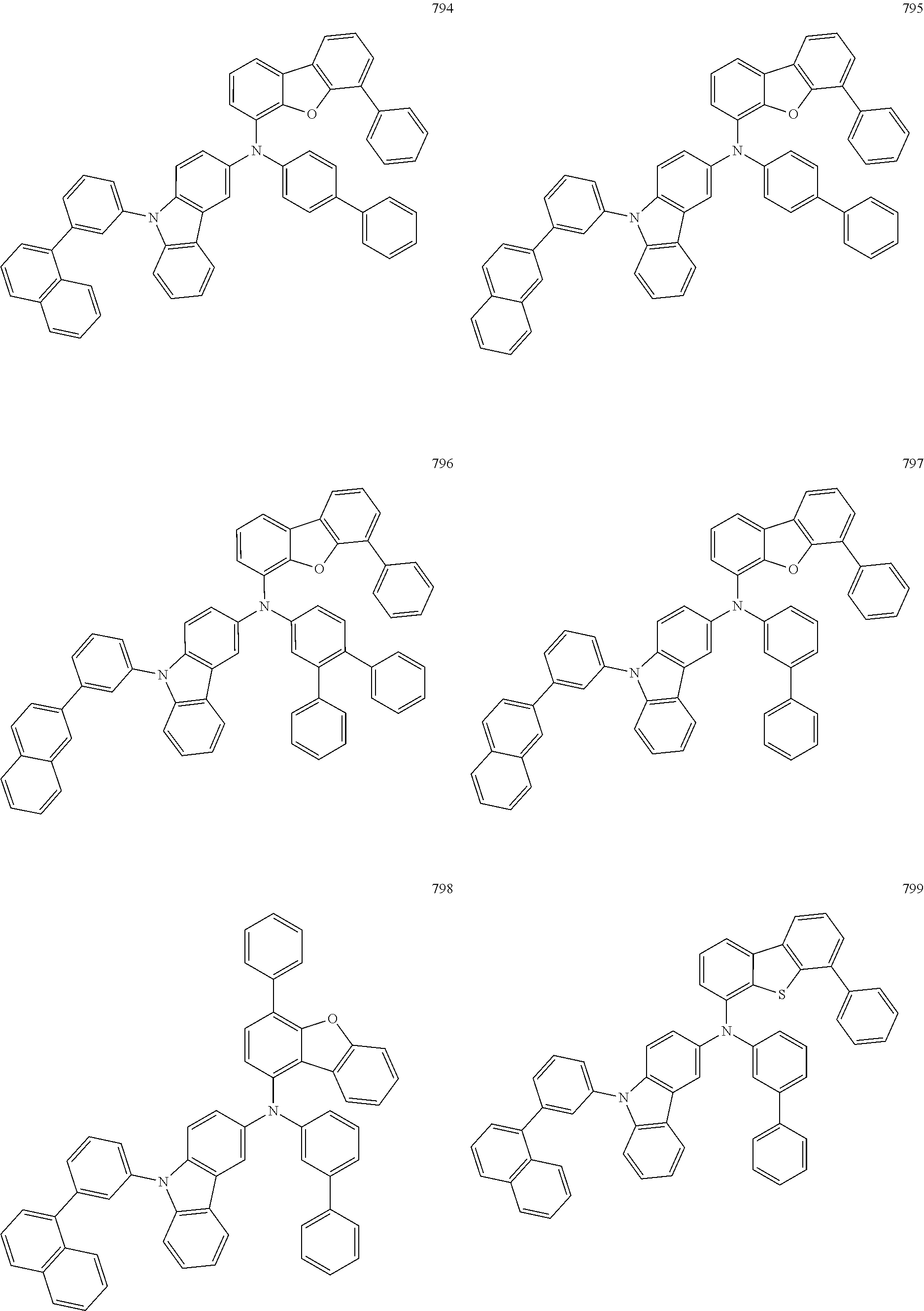 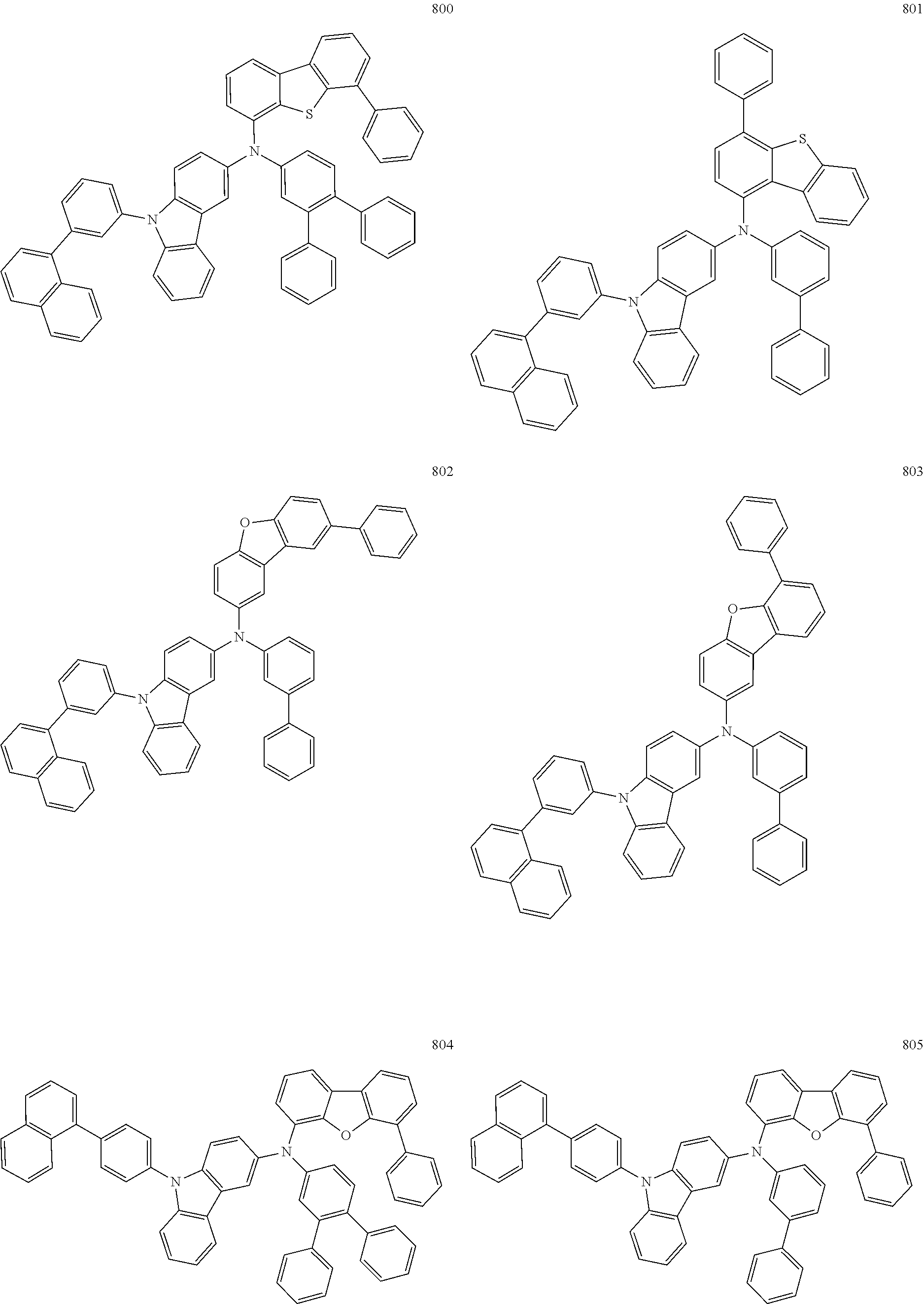 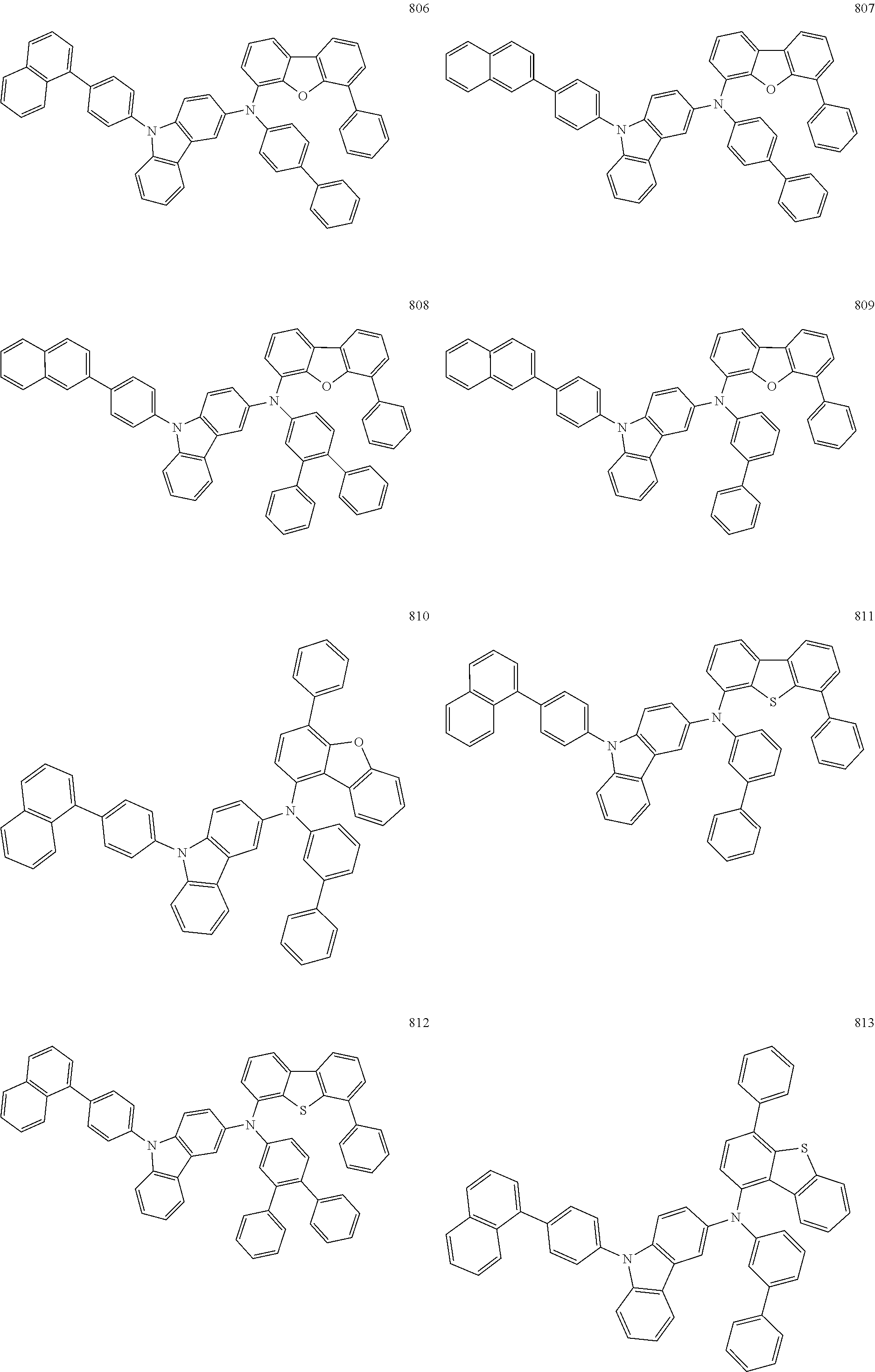 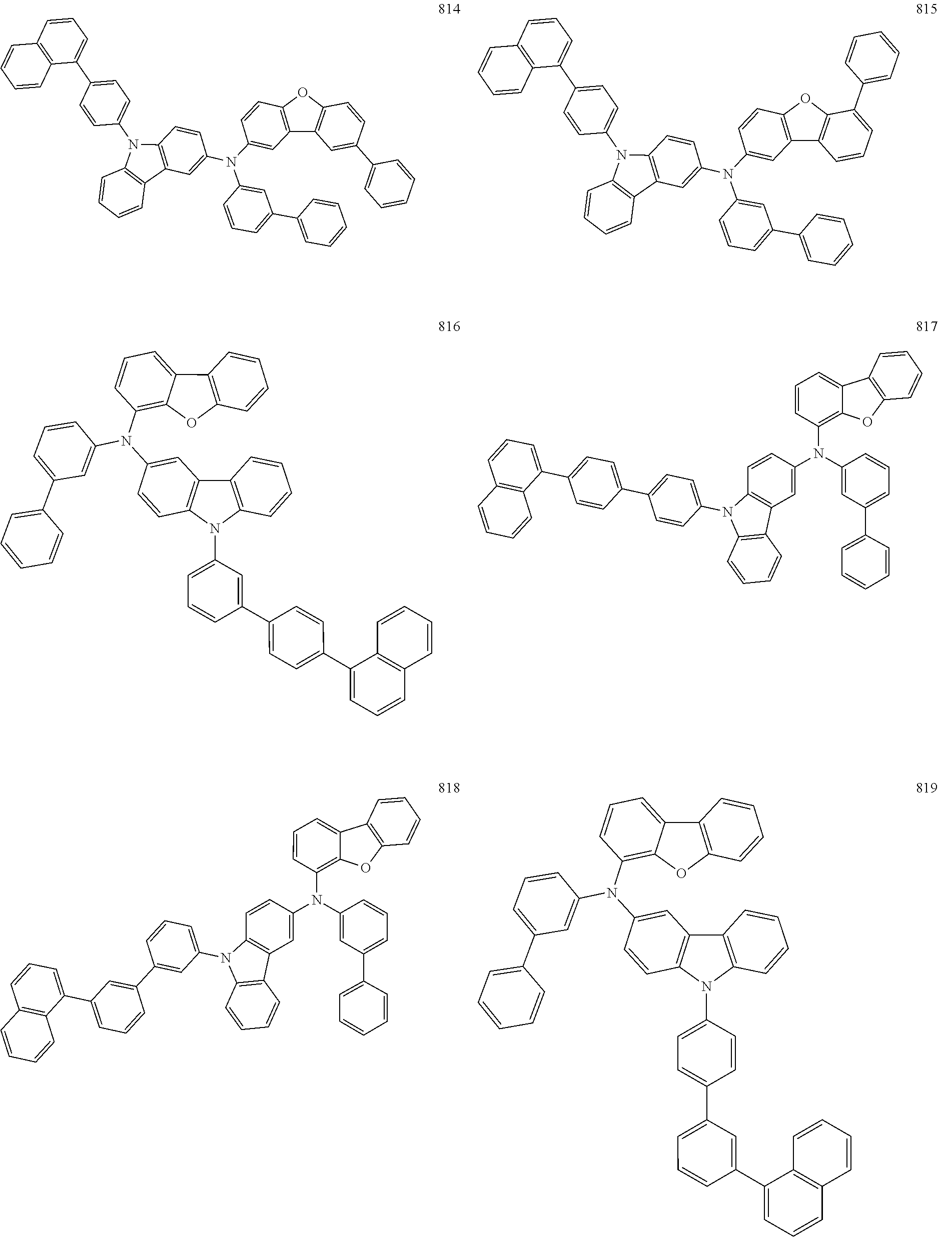 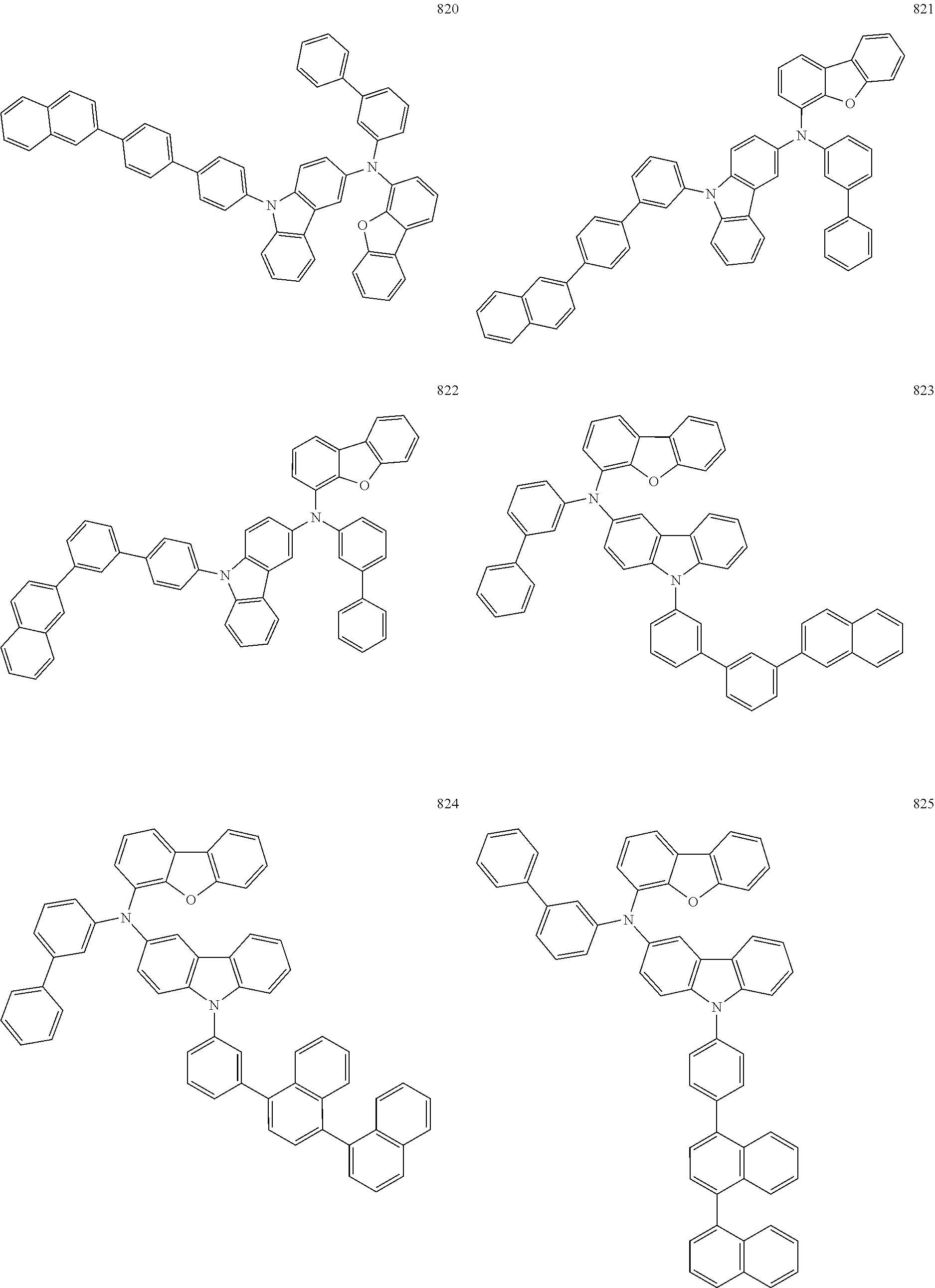 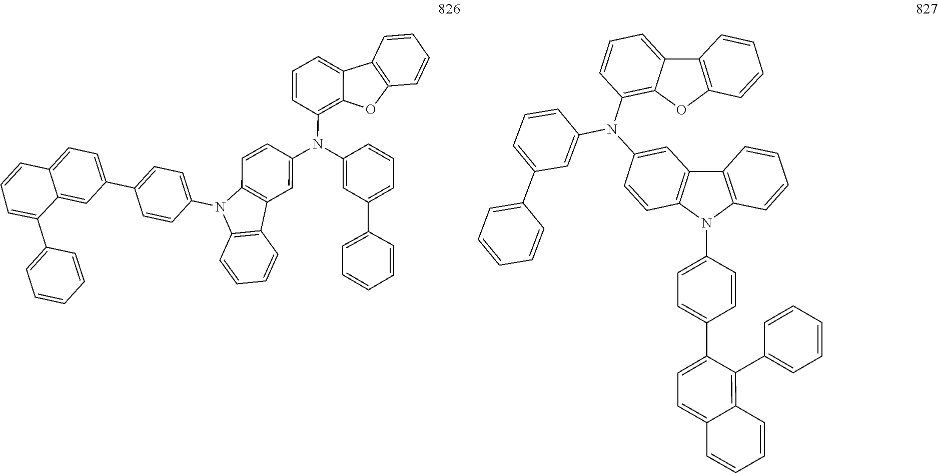 [SMILES: c1ccc(-c2cccc(N(c3ccc4c(c3)c3ccccc3n4-c3ccc(-c4ccc5cccc(-c6ccccc6)c5c4)cc3)c3cccc4c3oc3ccccc34)c2)cc1 ; c1ccc(-c2cccc(N(c3ccc4c(c3)c3ccccc3n4-c3ccc(-c4ccc5ccccc5c4-c4ccccc4)cc3)c3cccc4c3oc3ccccc34)c2)cc1] 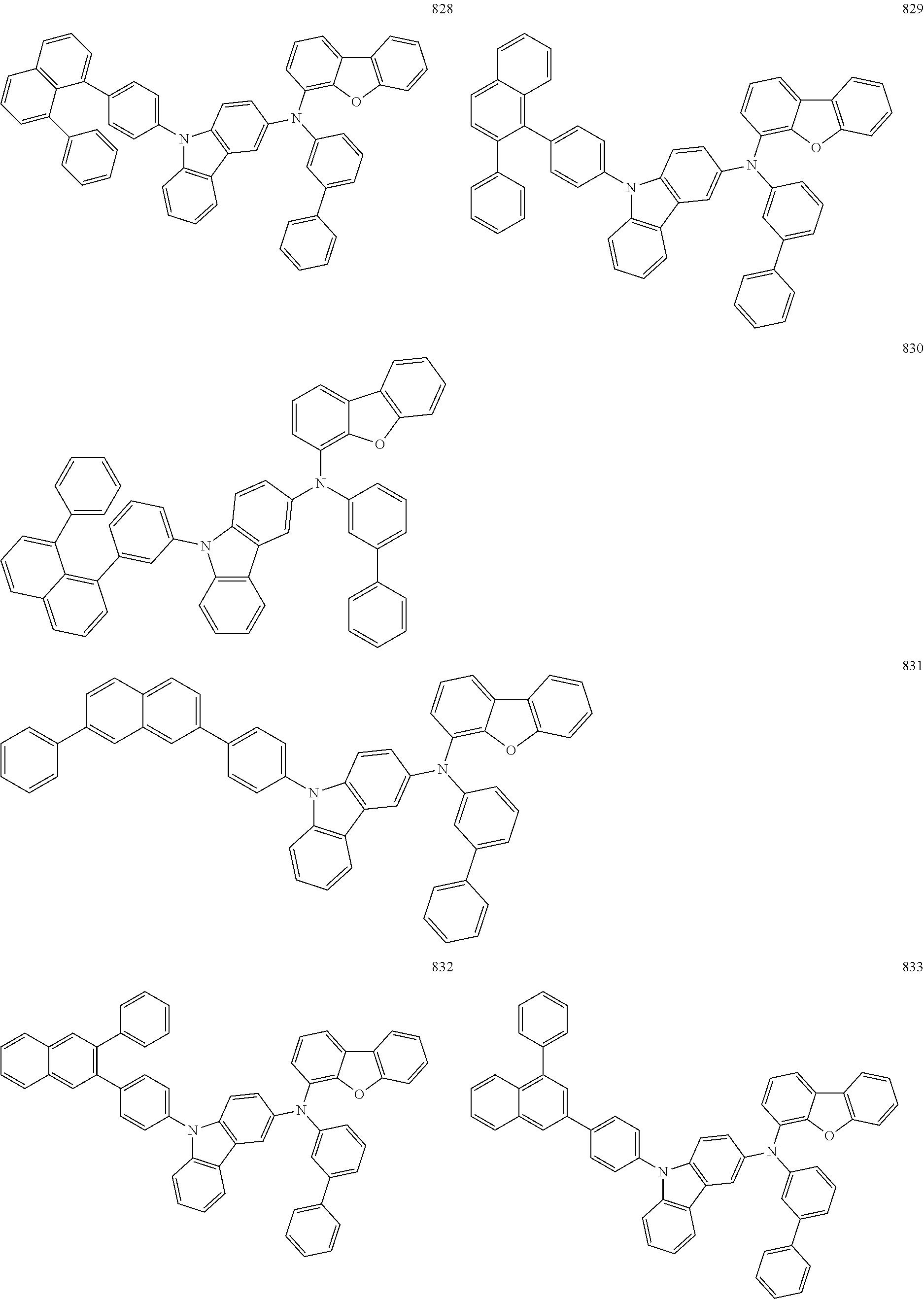 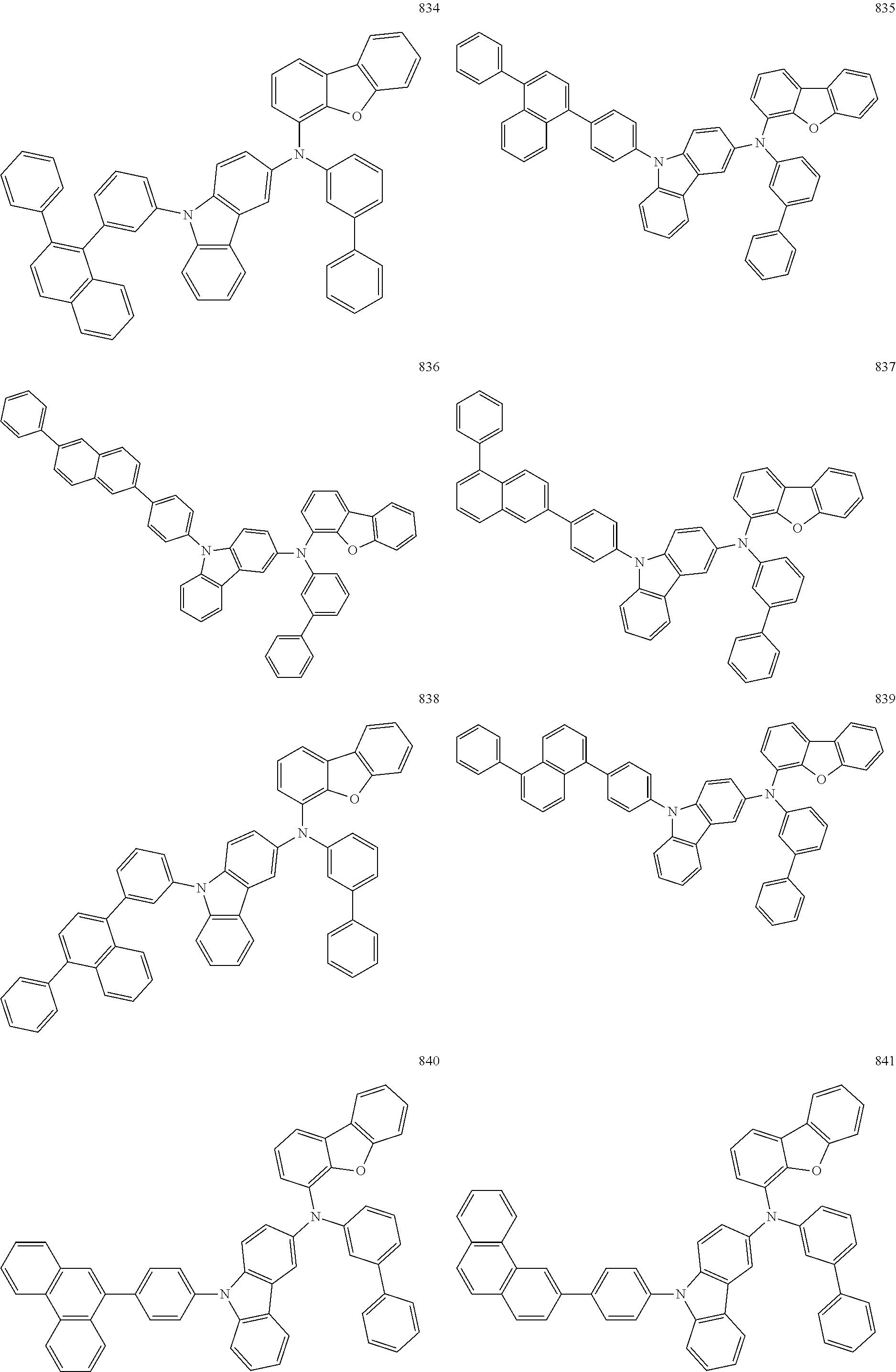 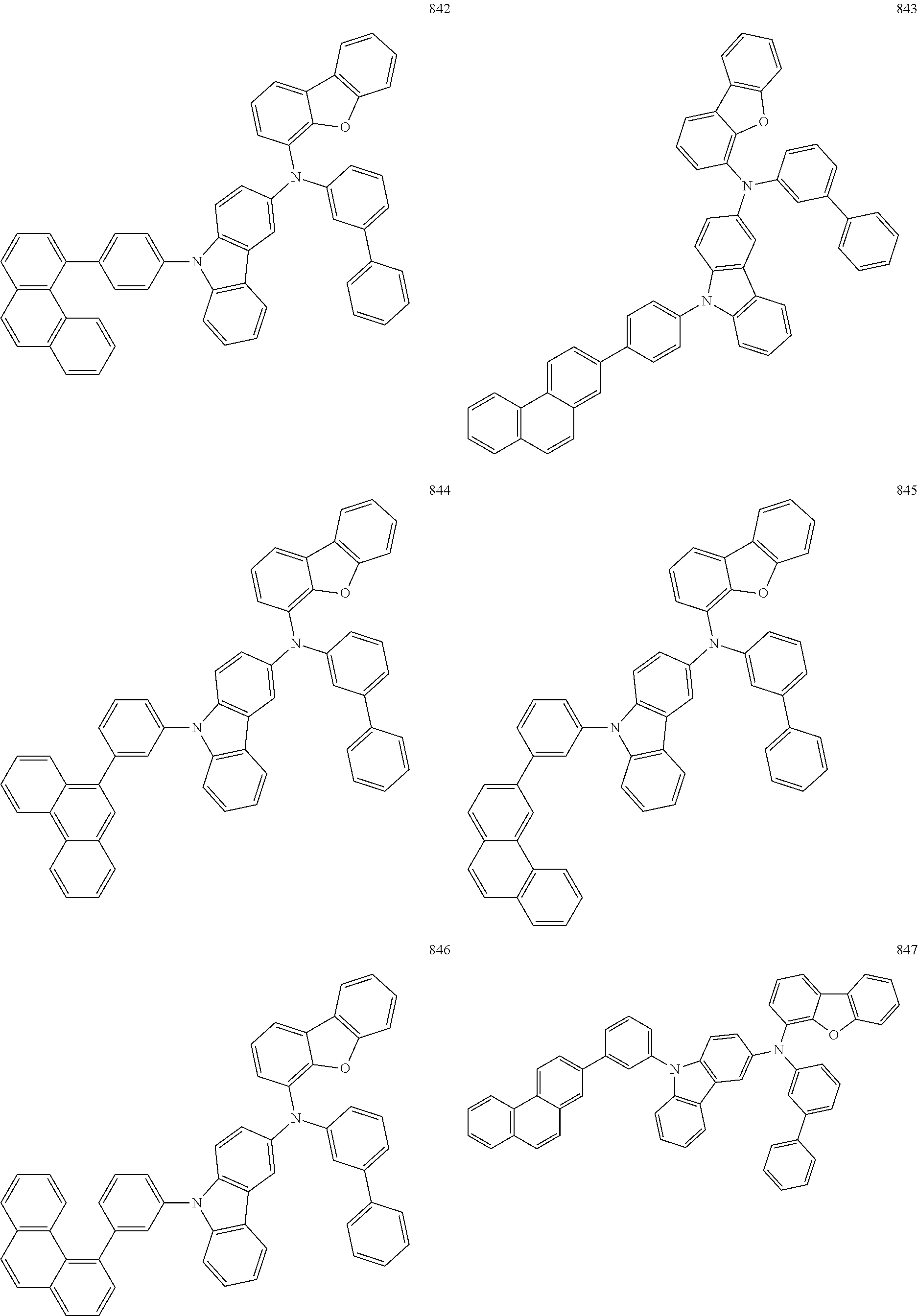 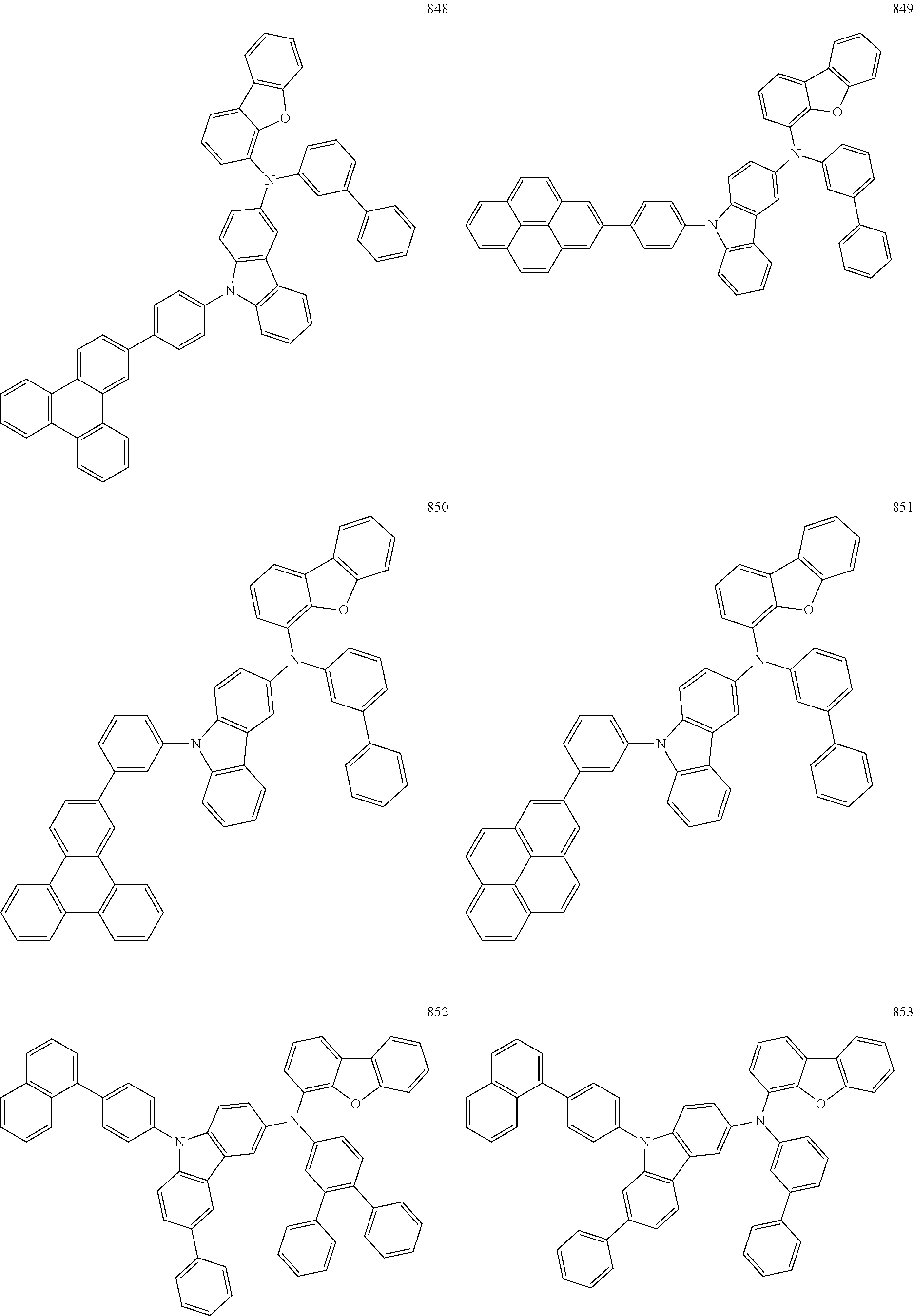 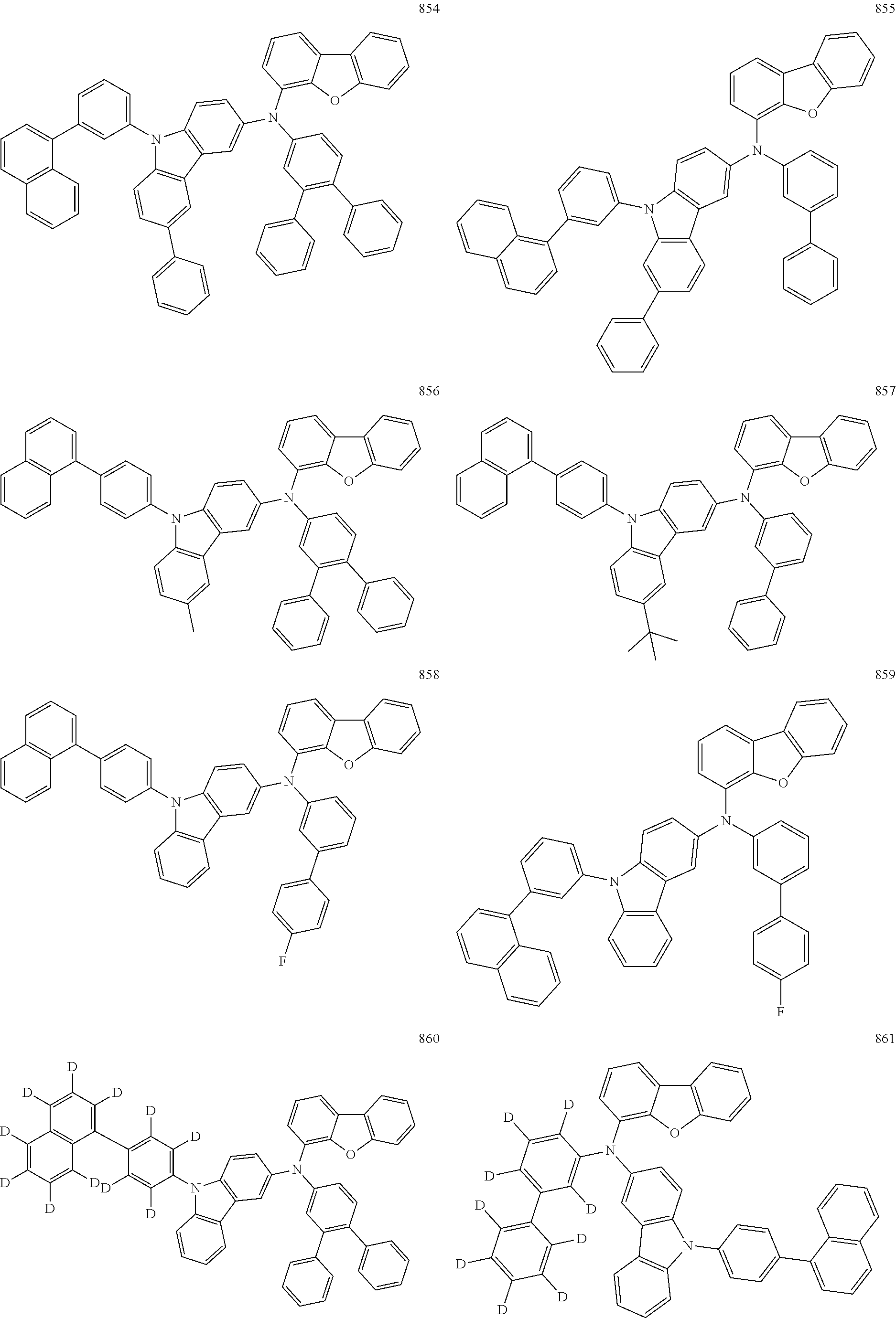 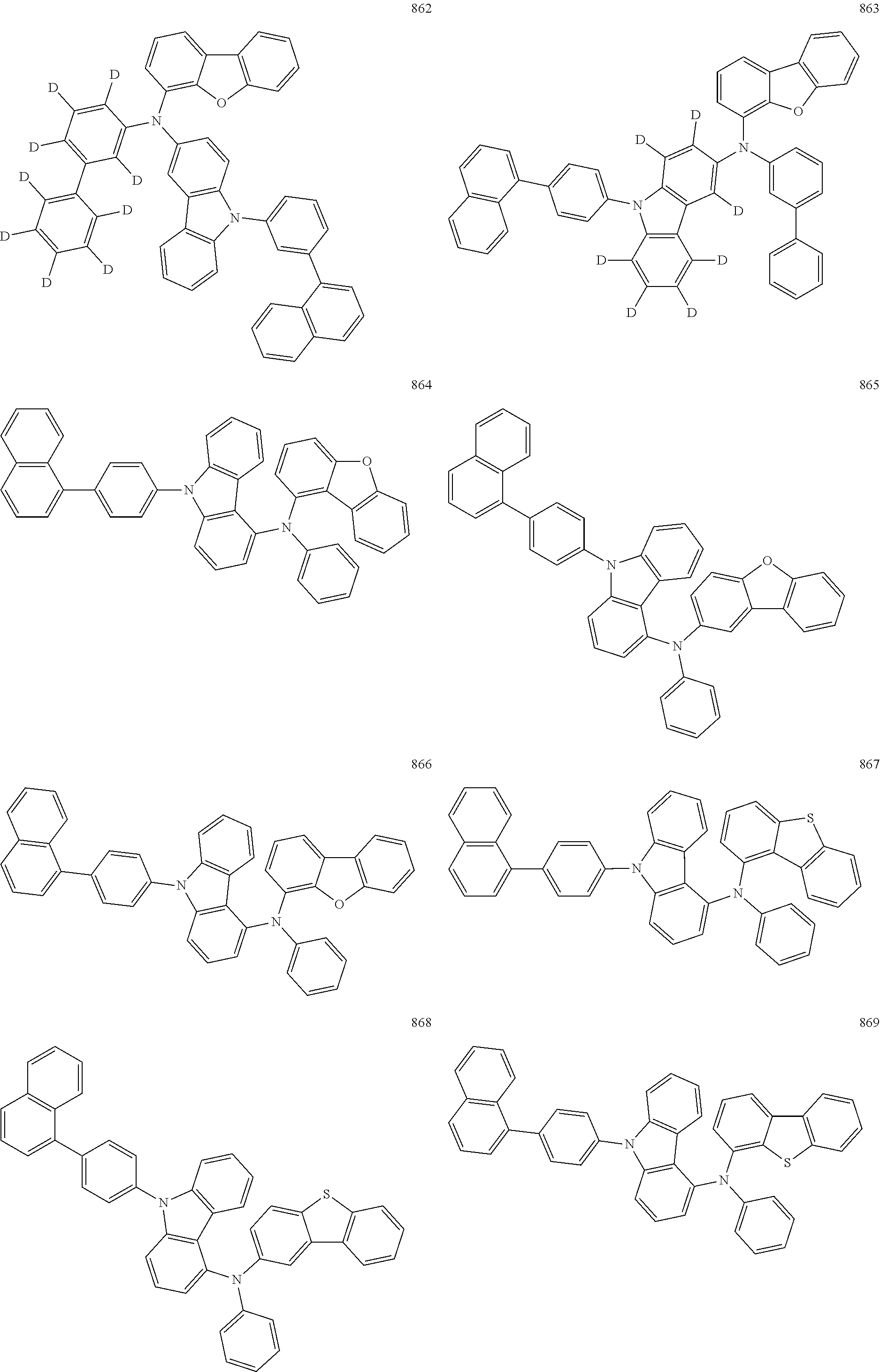 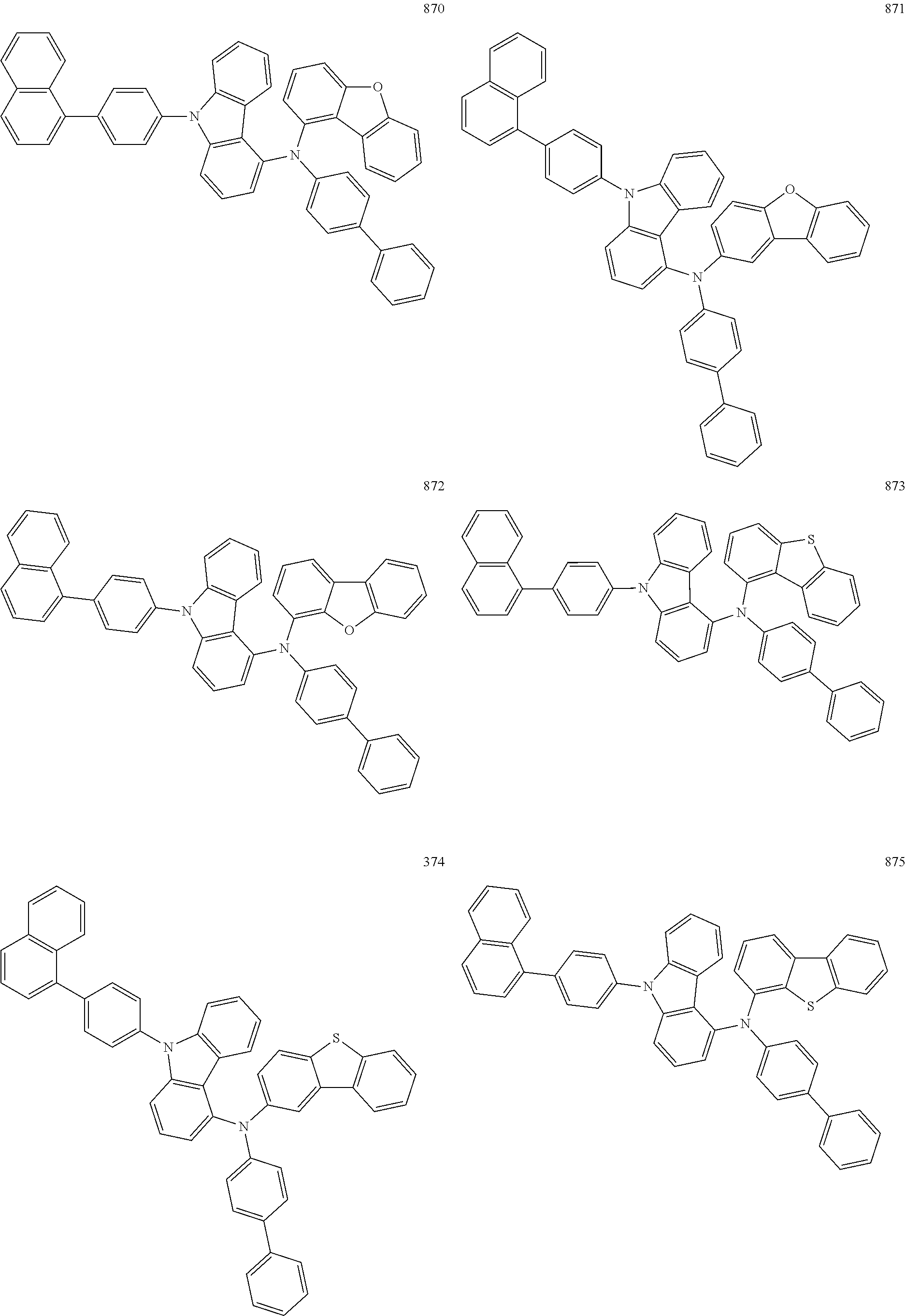 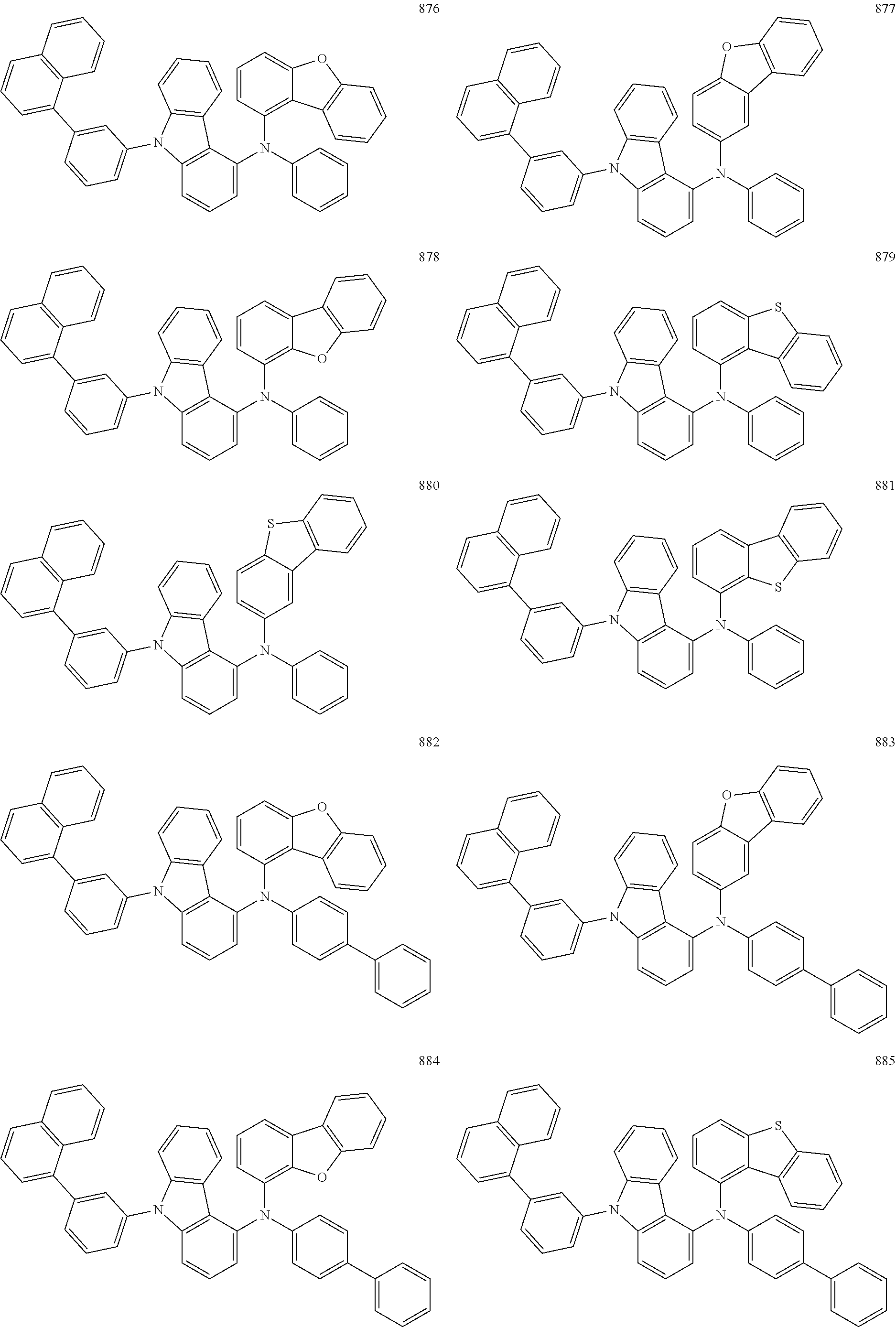 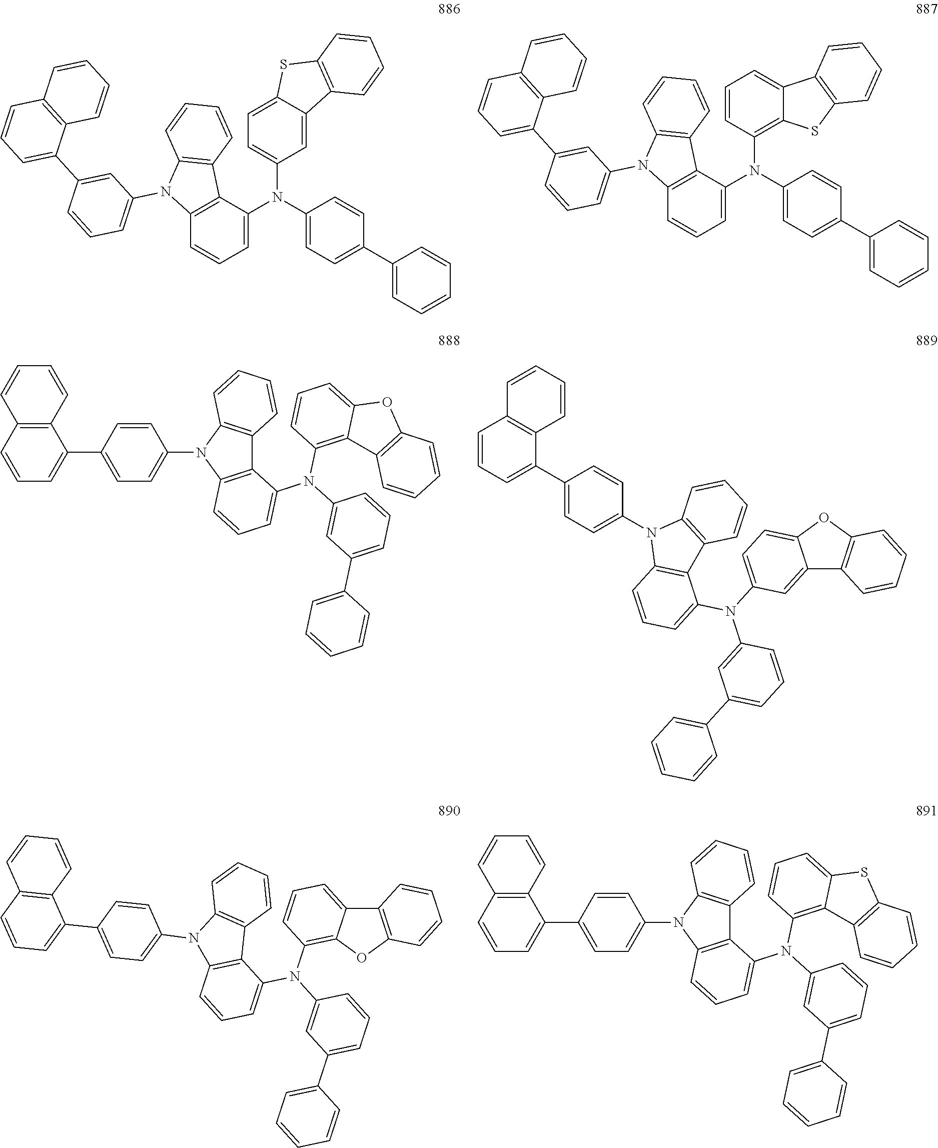 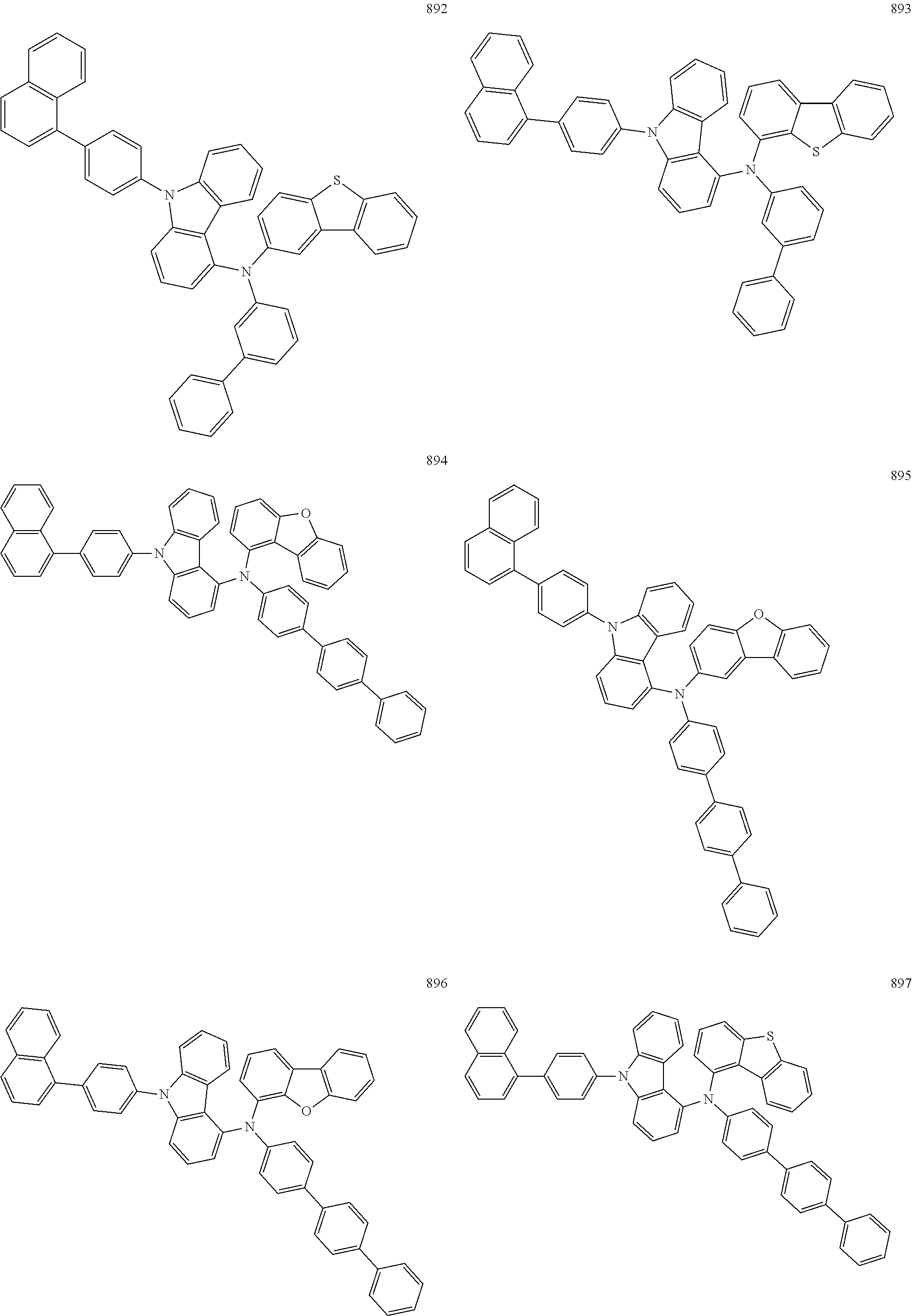 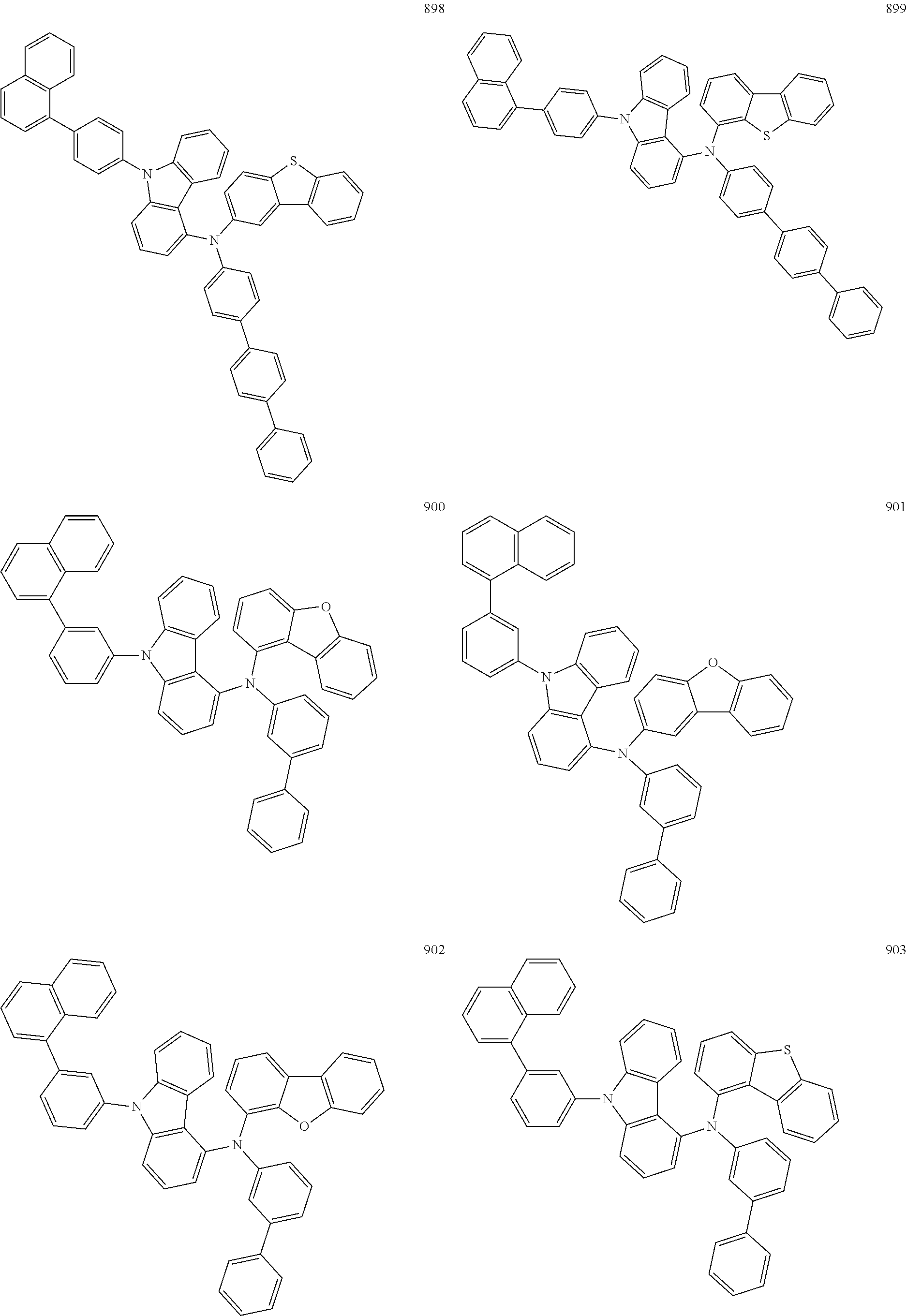 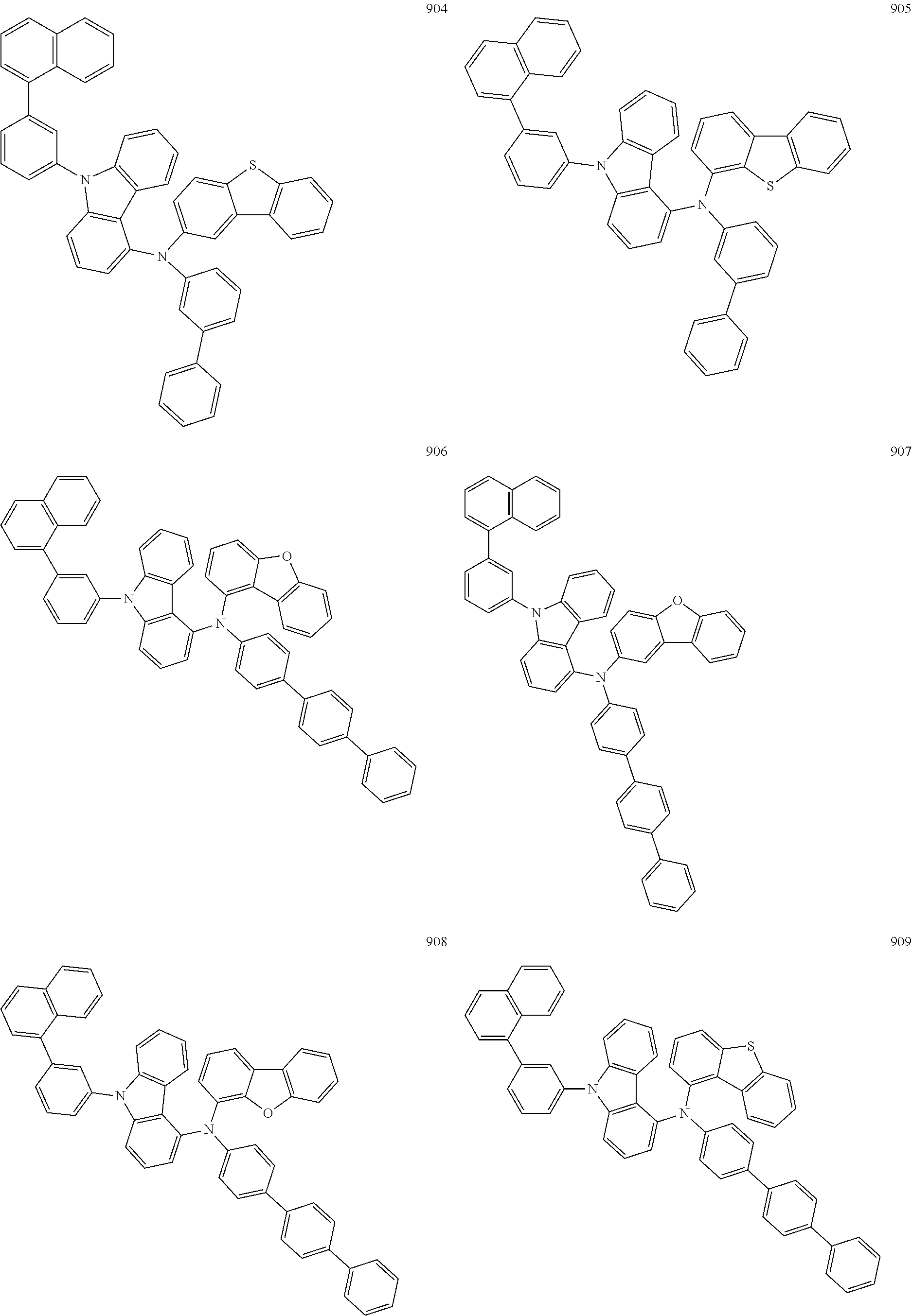 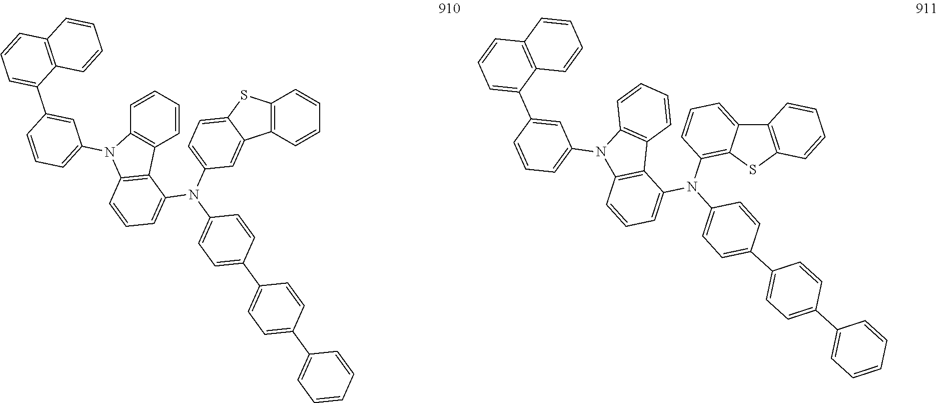 [SMILES: c1ccc(-c2ccc(-c3ccc(N(c4ccc5sc6ccccc6c5c4)c4cccc5c4c4ccccc4n5-c4cccc(-c5cccc6ccccc56)c4)cc3)cc2)cc1 ; c1ccc(-c2ccc(-c3ccc(N(c4cccc5c4sc4ccccc45)c4cccc5c4c4ccccc4n5-c4cccc(-c5cccc6ccccc56)c4)cc3)cc2)cc1] 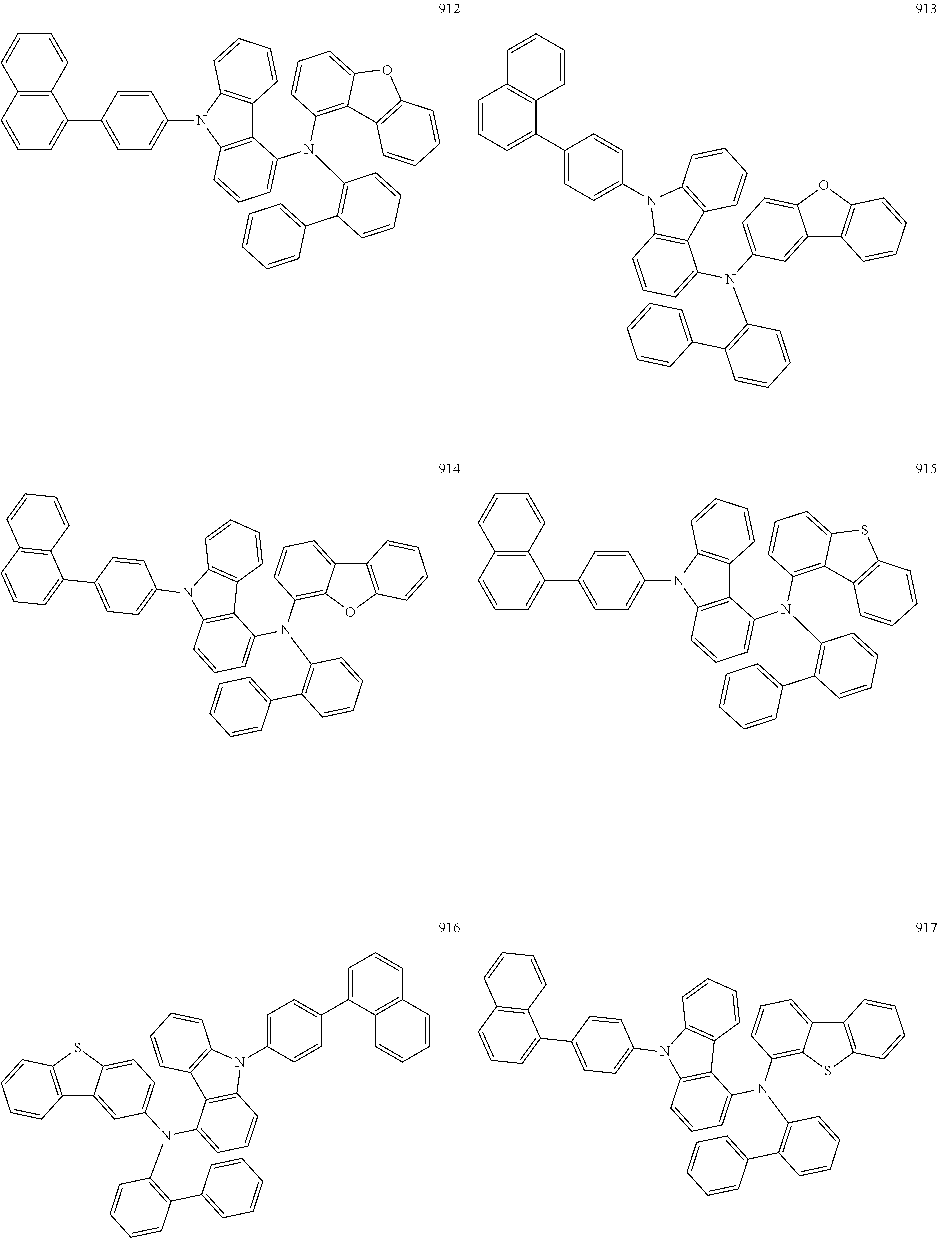 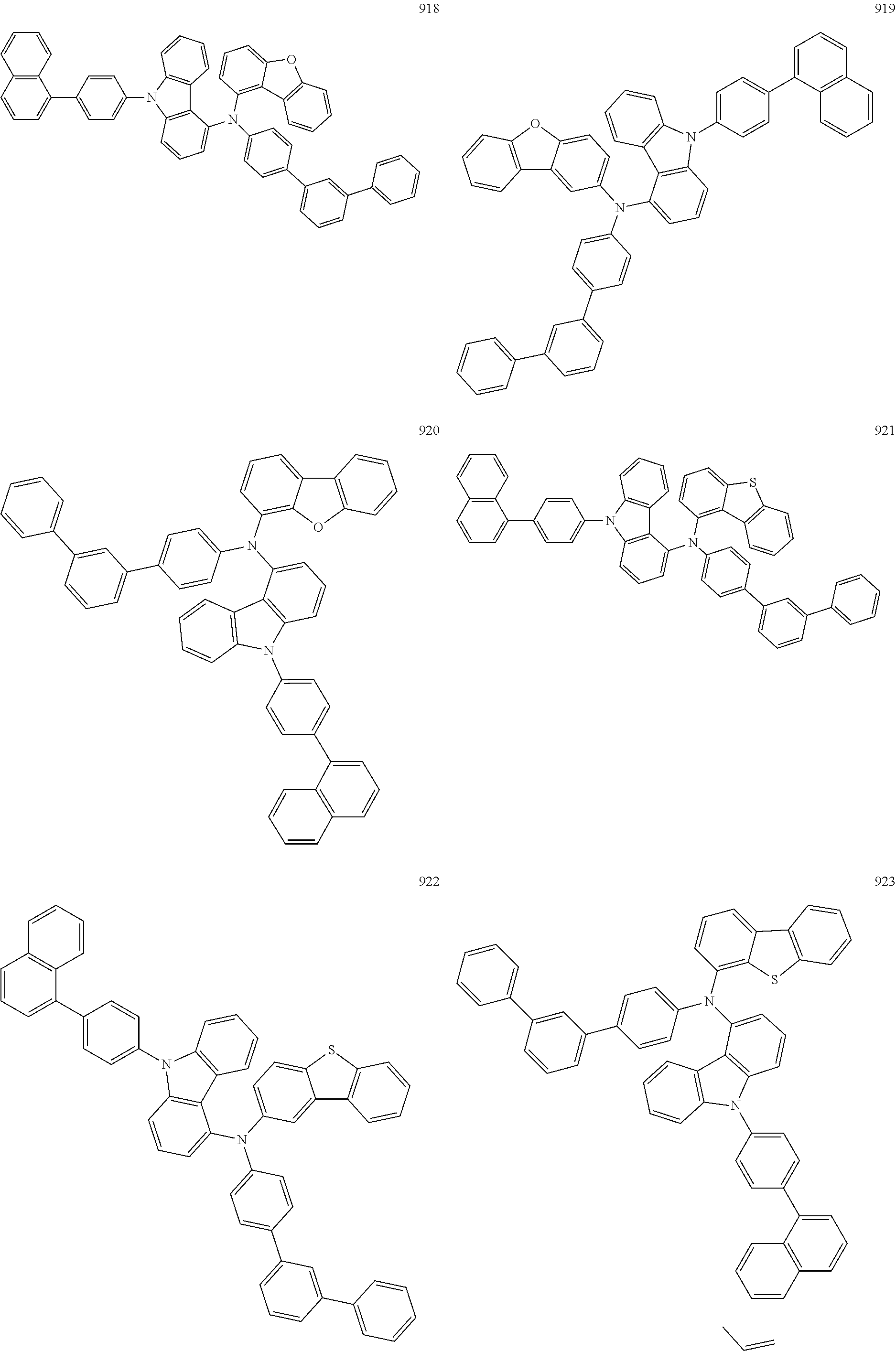 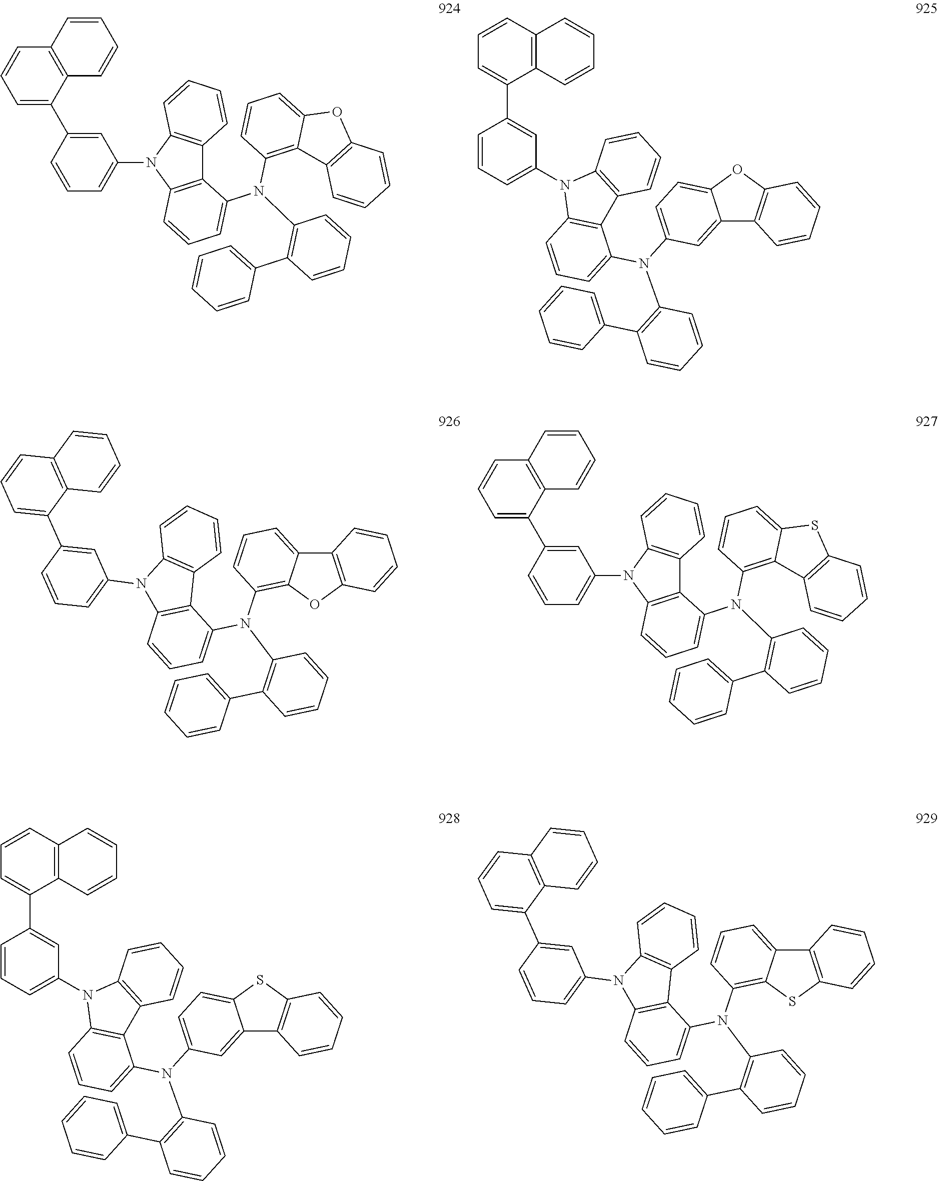 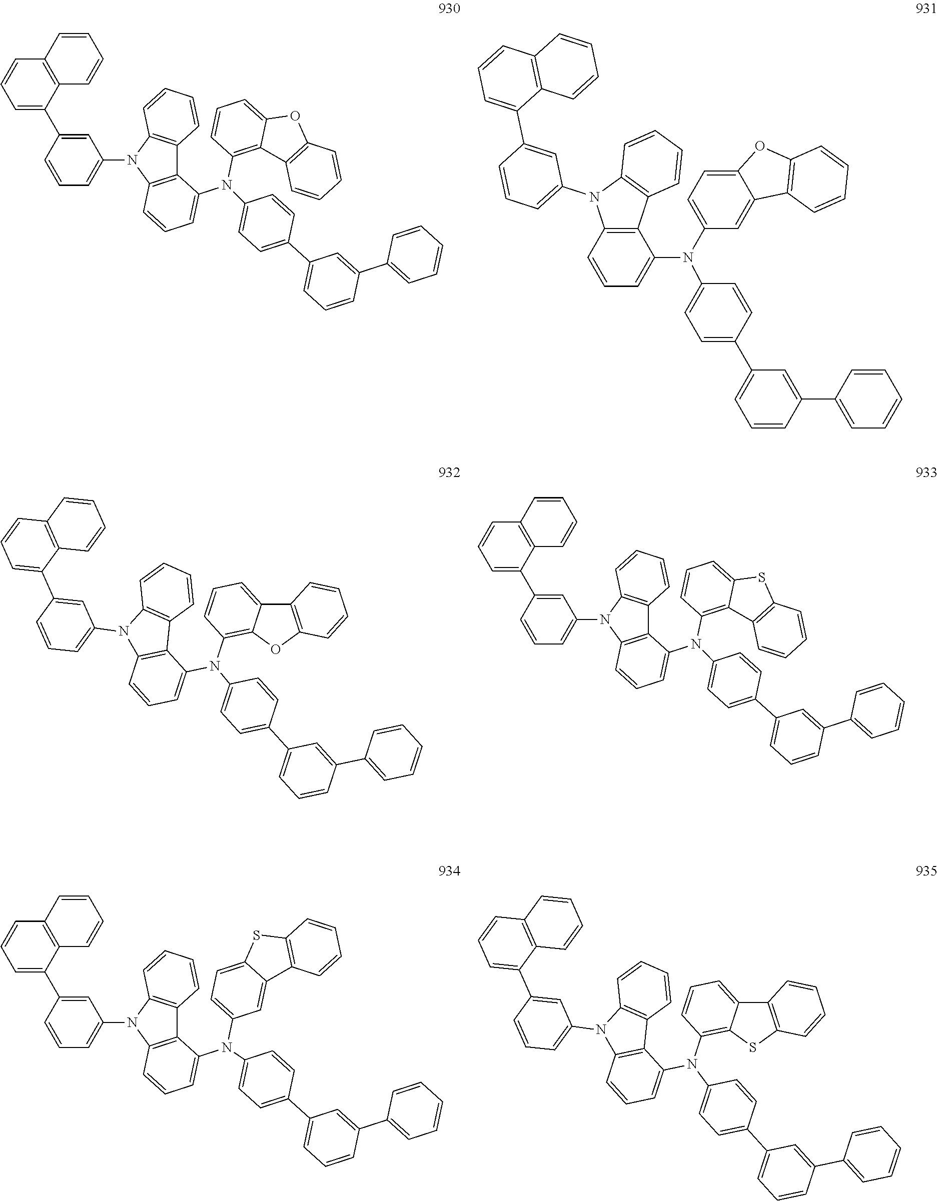 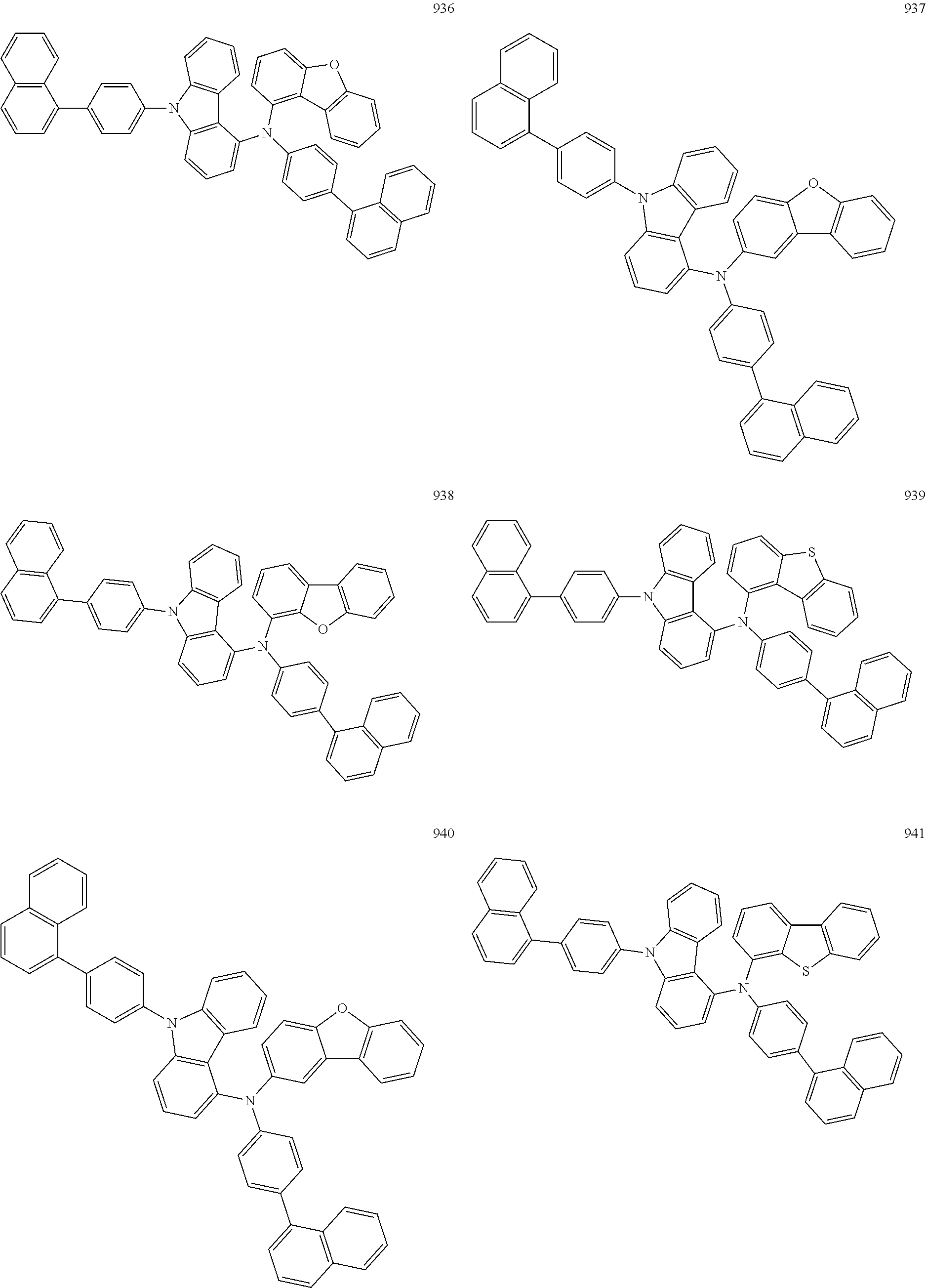 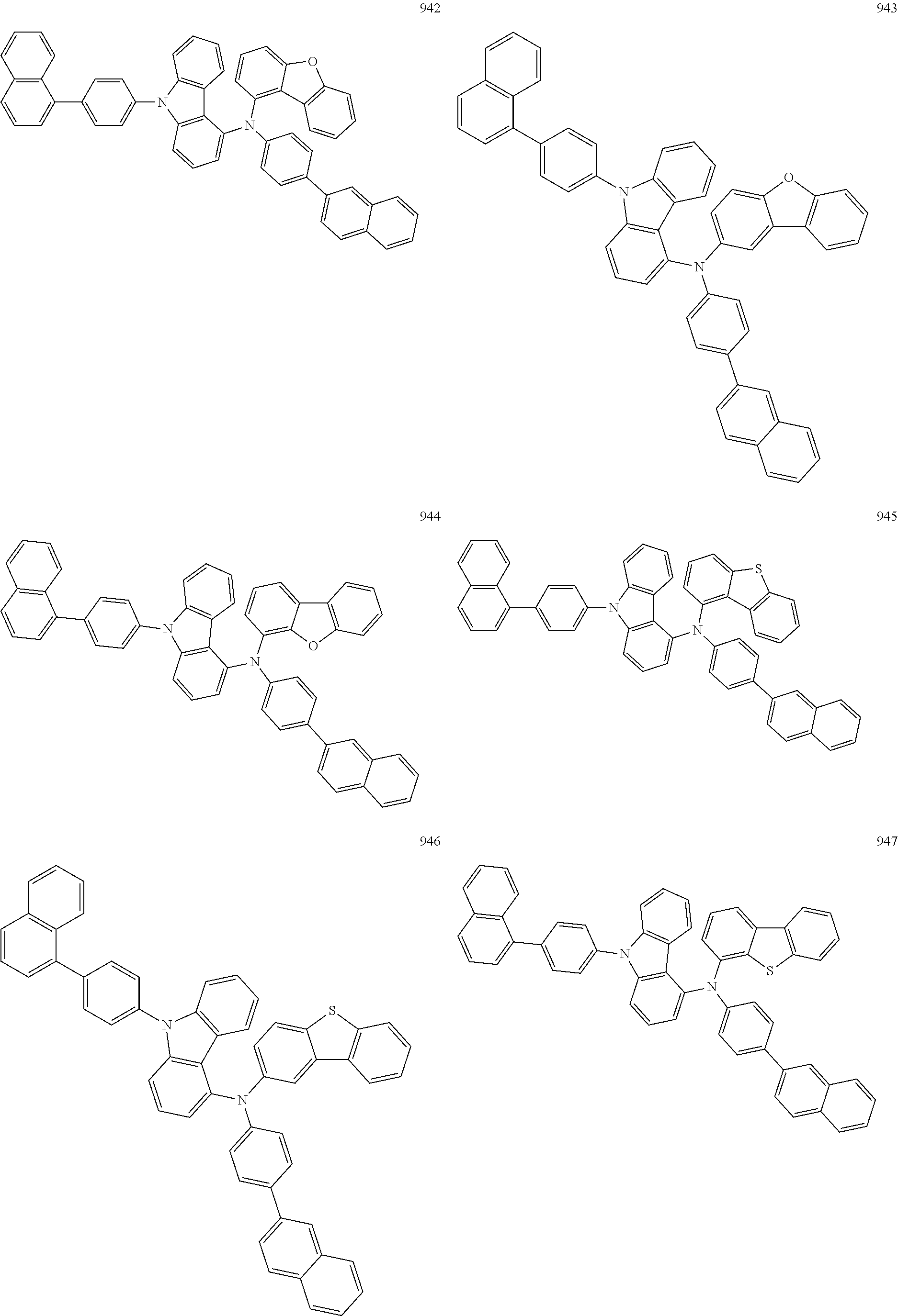 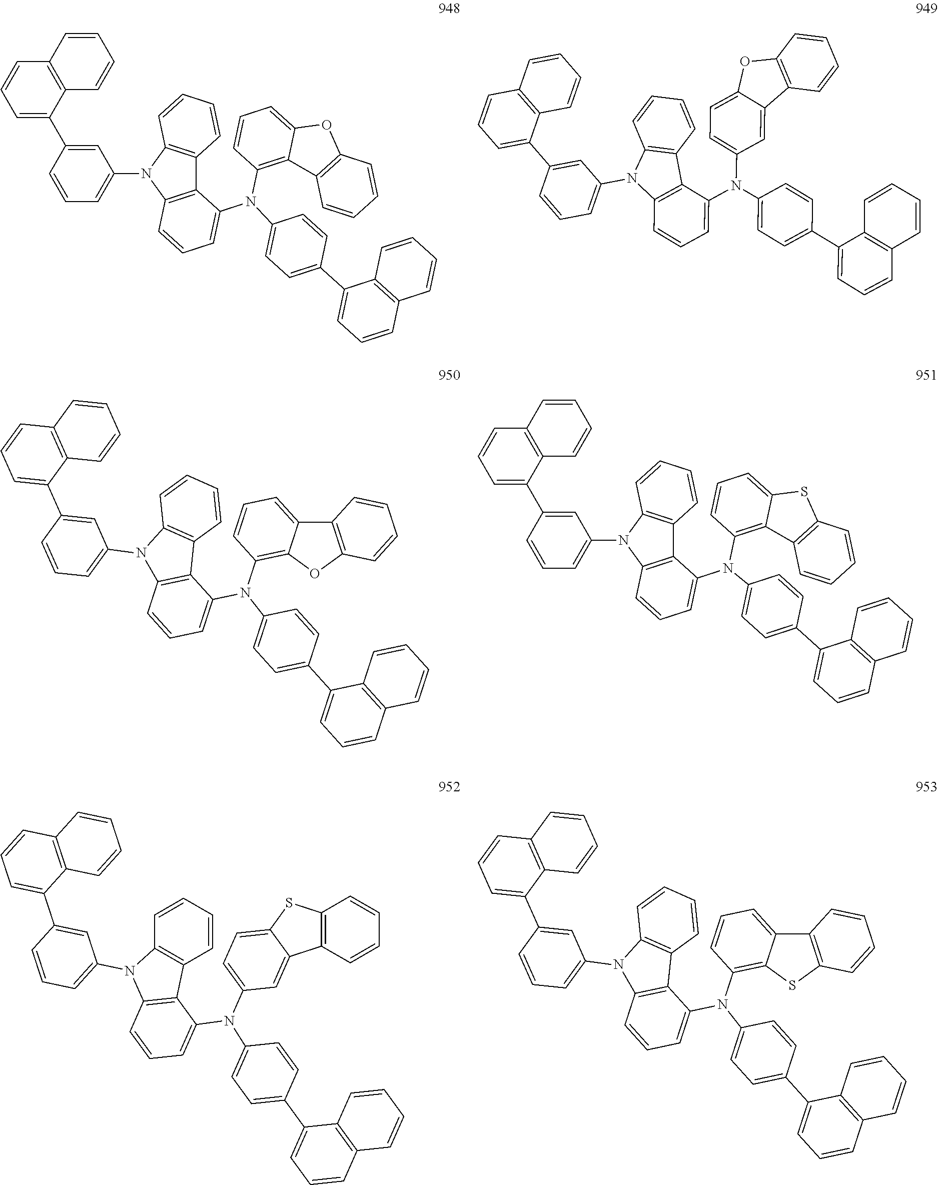 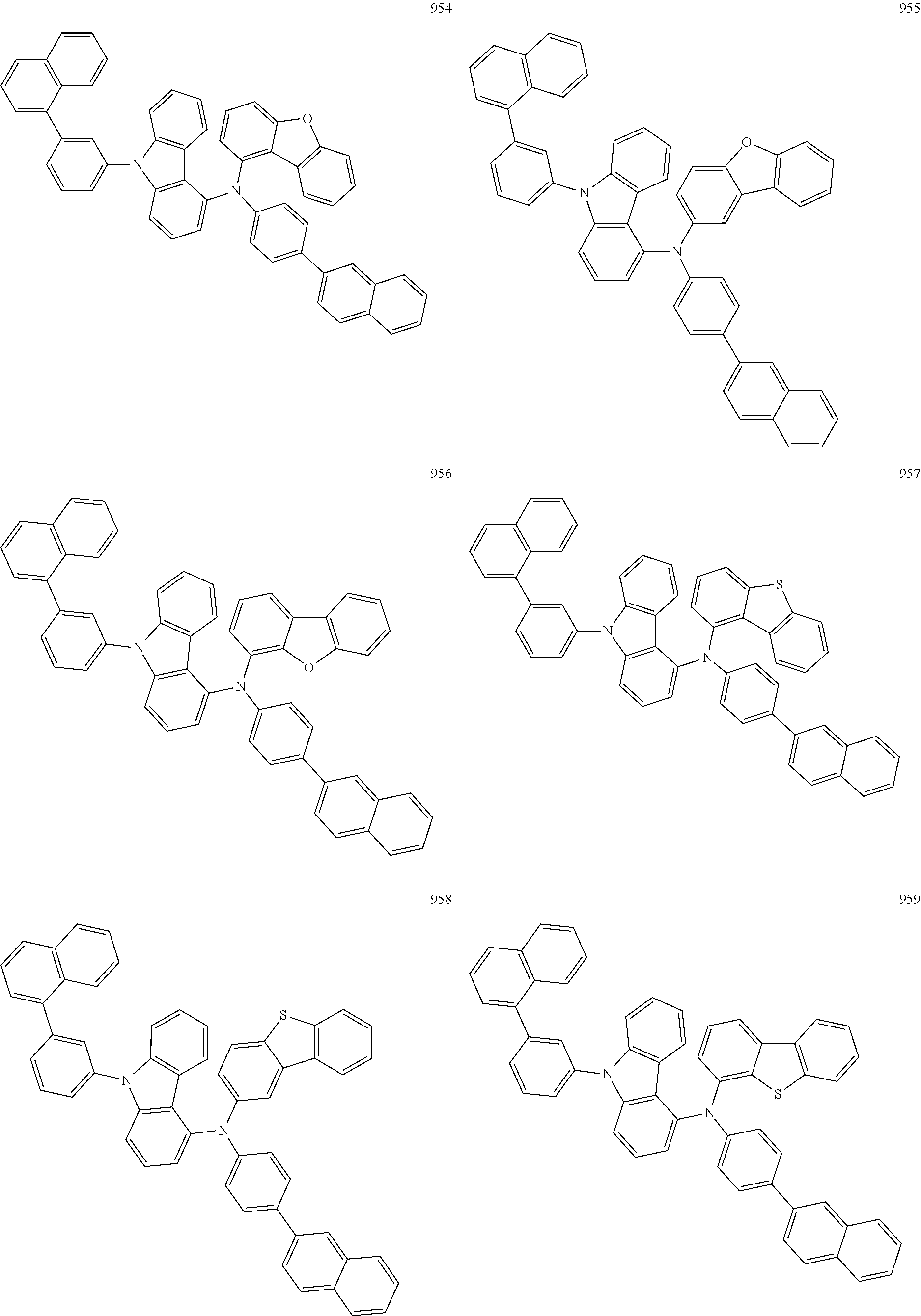 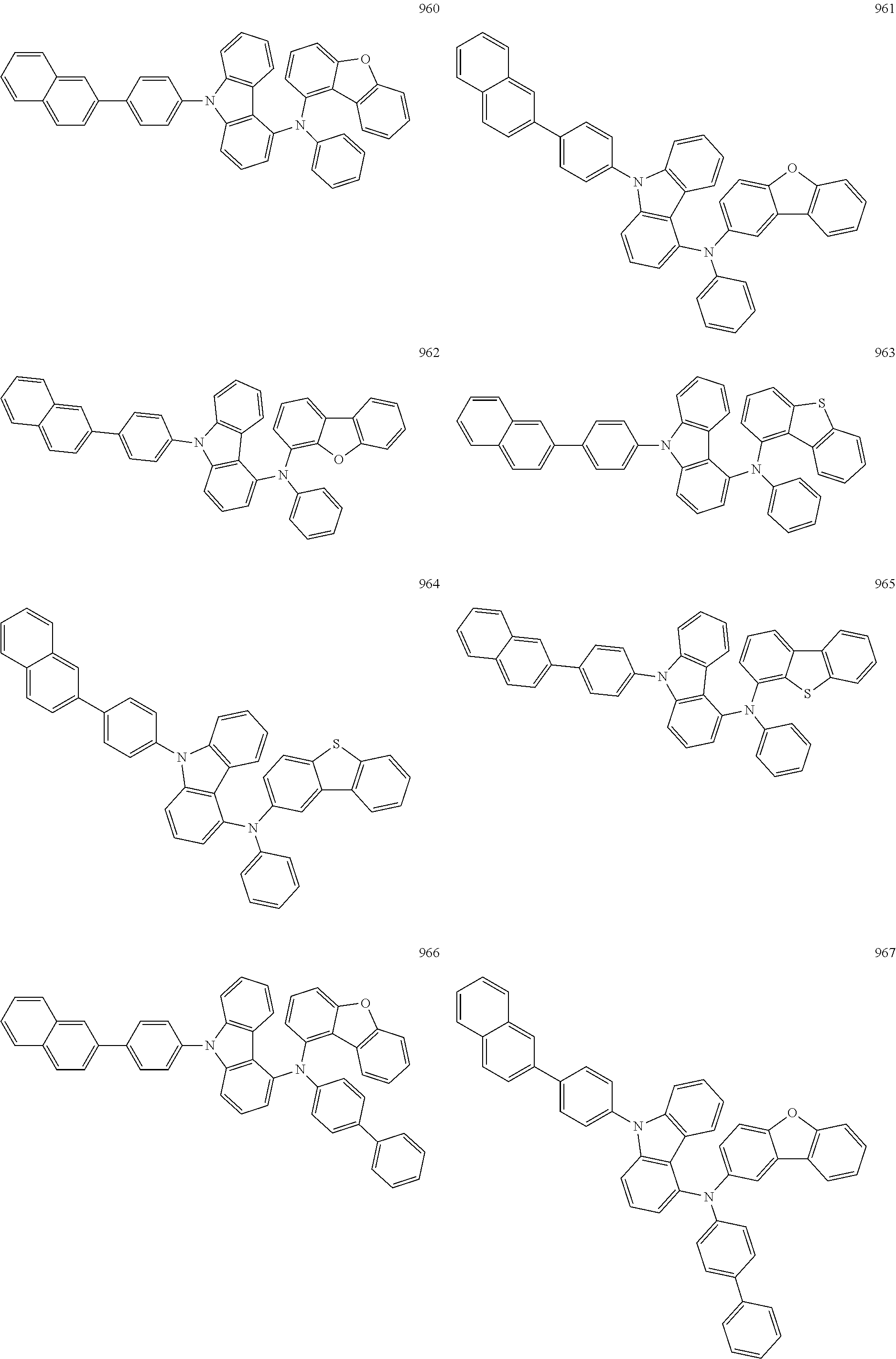 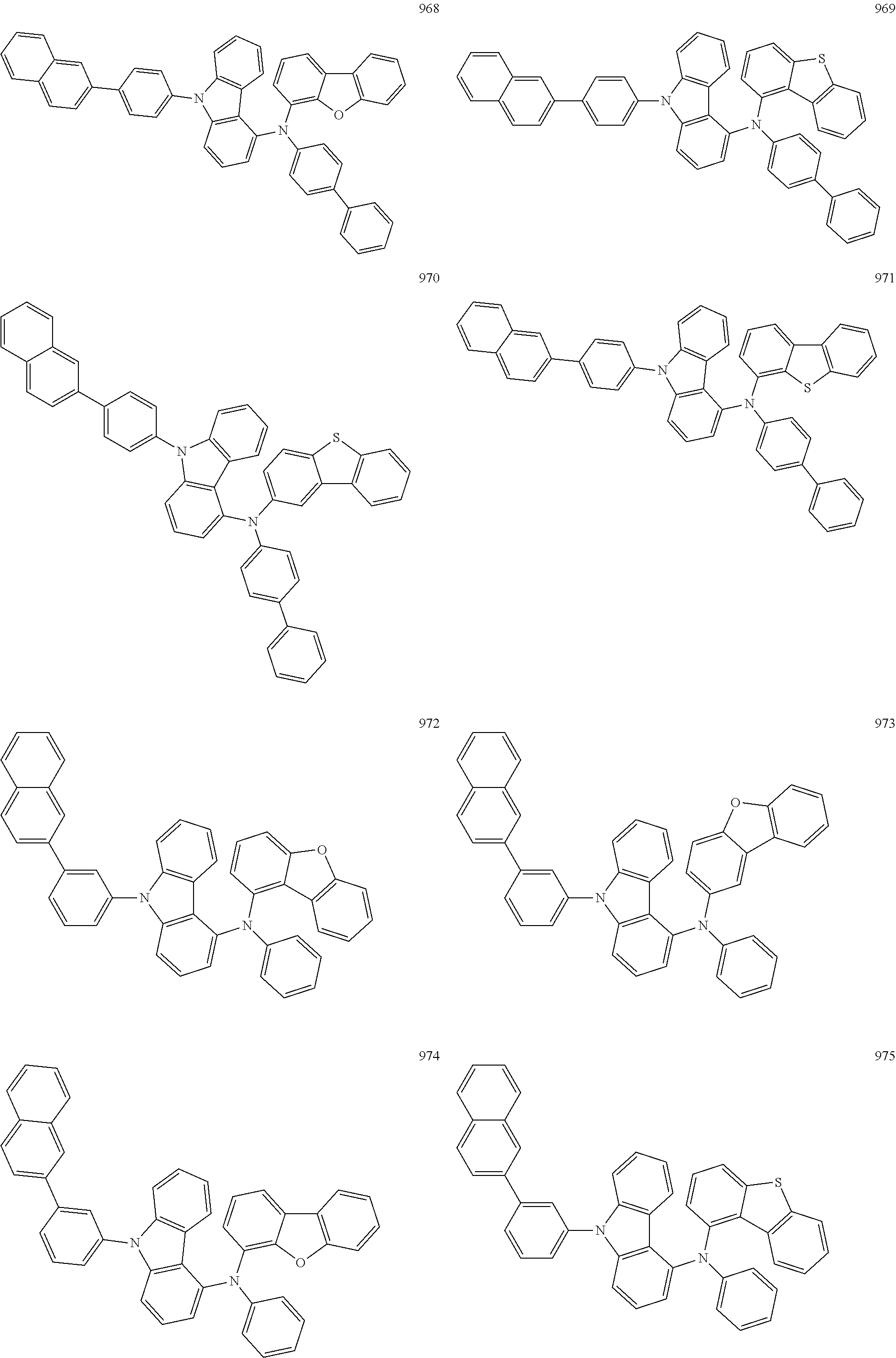 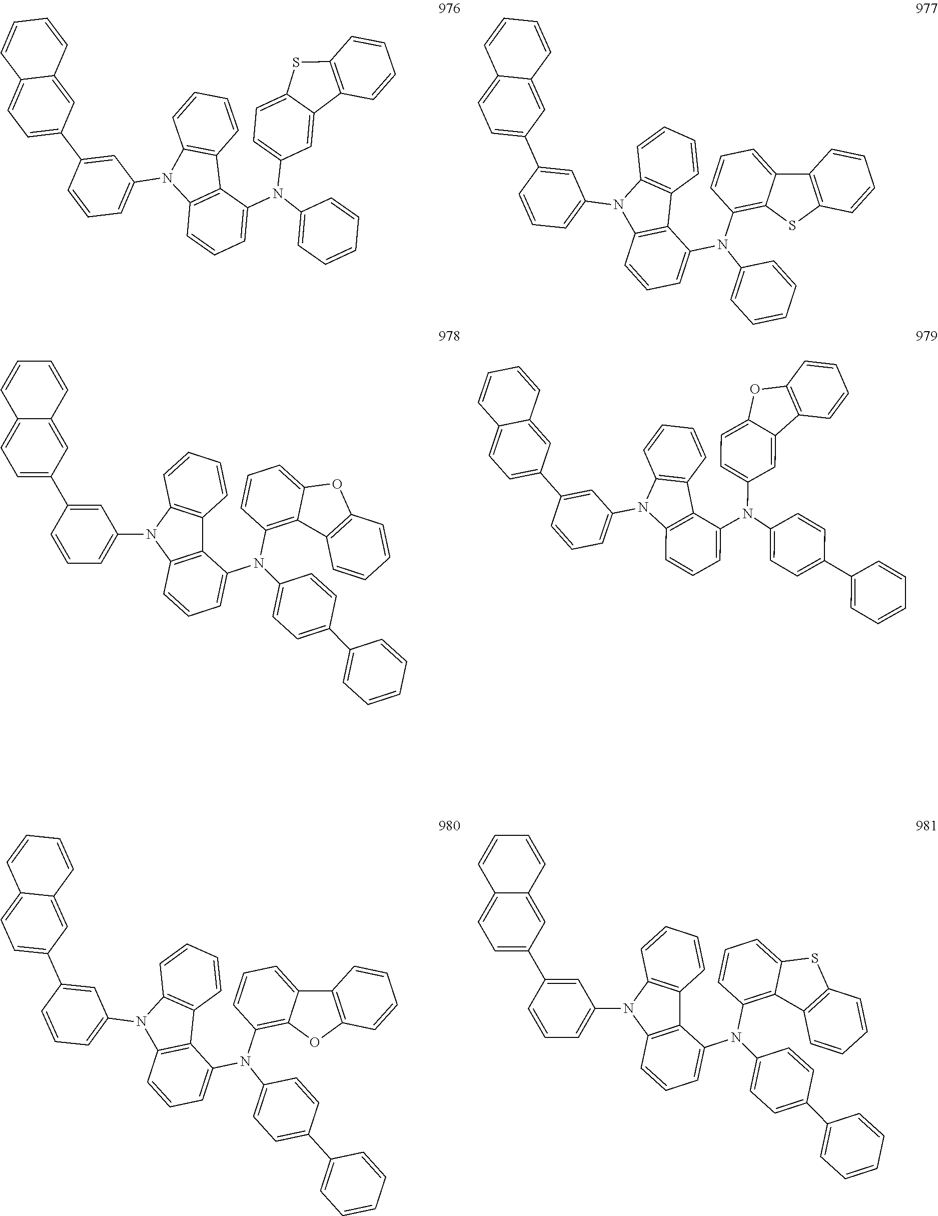 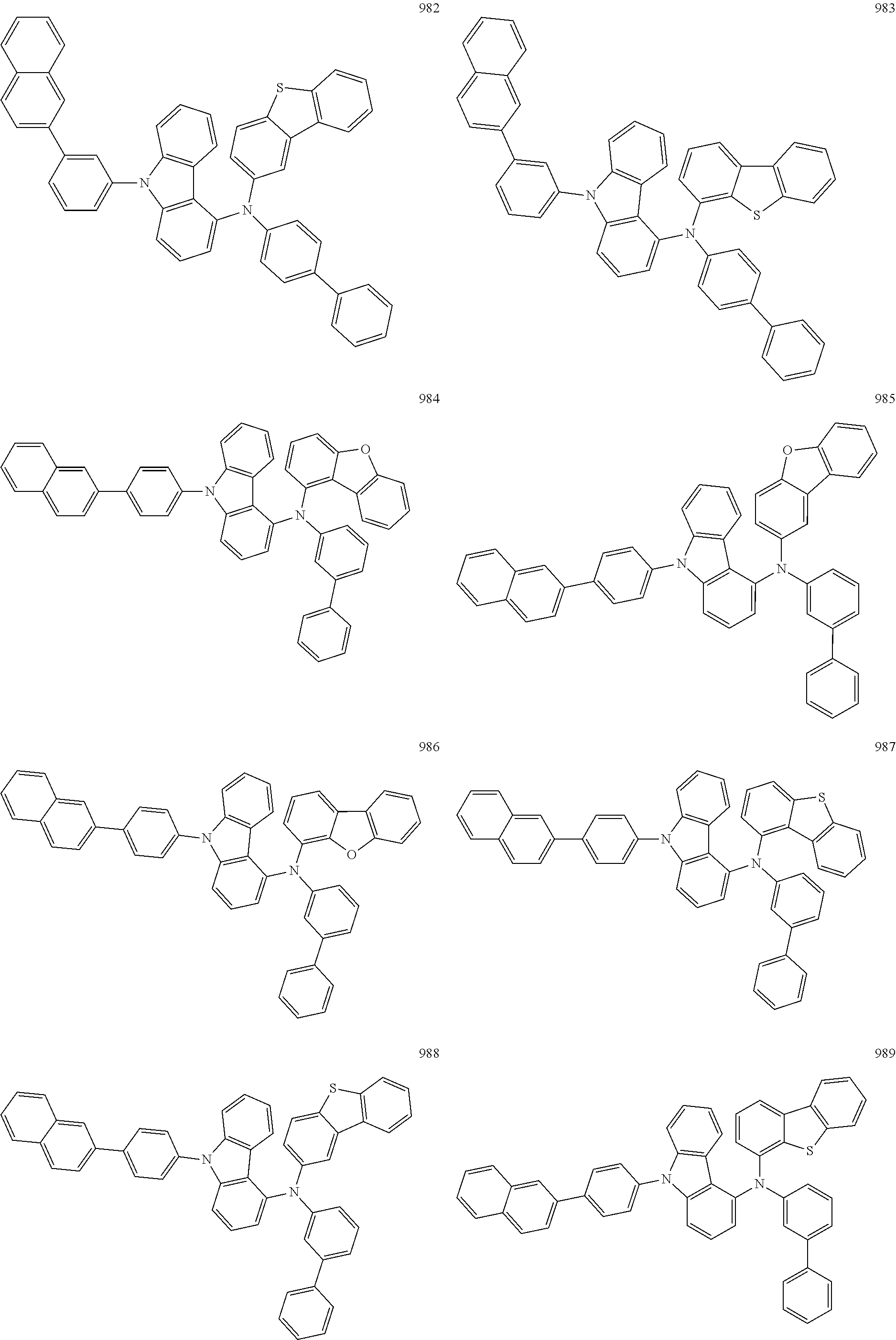 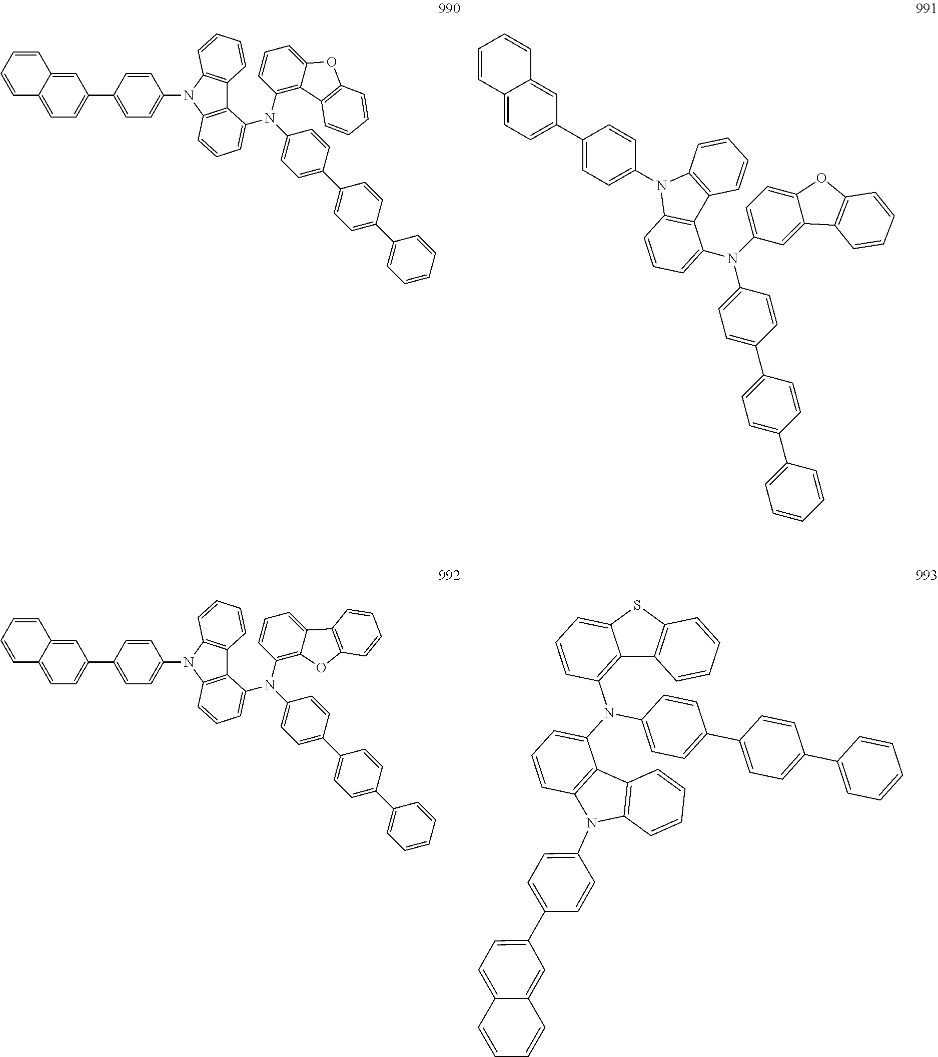 [SMILES: c1ccc(-c2ccc(-c3ccc(N(c4cccc5oc6ccccc6c45)c4cccc5c4c4ccccc4n5-c4ccc(-c5ccc6ccccc6c5)cc4)cc3)cc2)cc1 ; c1ccc(-c2ccc(-c3ccc(N(c4ccc5oc6ccccc6c5c4)c4cccc5c4c4ccccc4n5-c4ccc(-c5ccc6ccccc6c5)cc4)cc3)cc2)cc1 ; c1ccc(-c2ccc(-c3ccc(N(c4cccc5c4oc4ccccc45)c4cccc5c4c4ccccc4n5-c4ccc(-c5ccc6ccccc6c5)cc4)cc3)cc2)cc1 ; c1ccc(-c2ccc(-c3ccc(N(c4cccc5sc6ccccc6c45)c4cccc5c4c4ccccc4n5-c4ccc(-c5ccc6ccccc6c5)cc4)cc3)cc2)cc1] 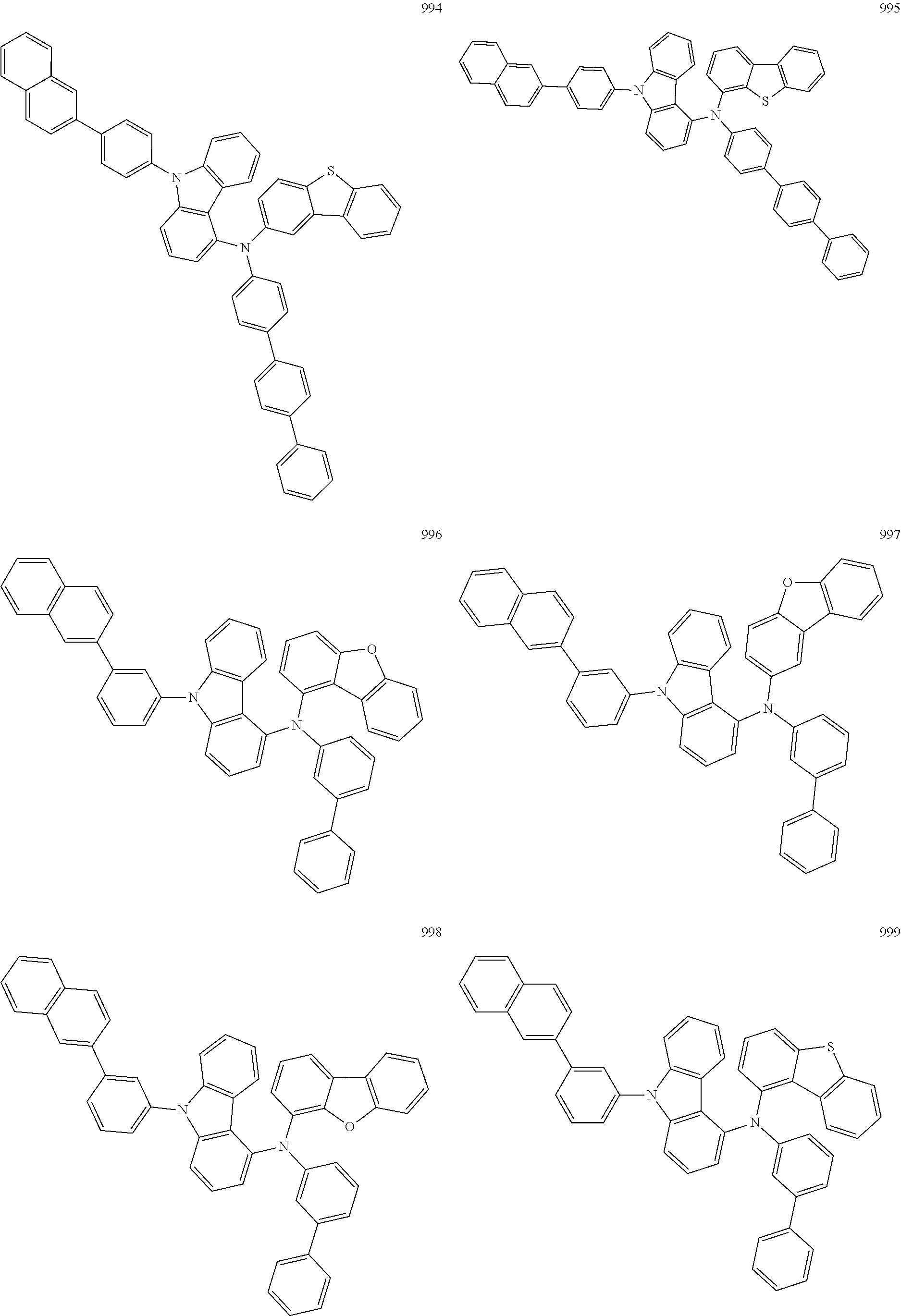 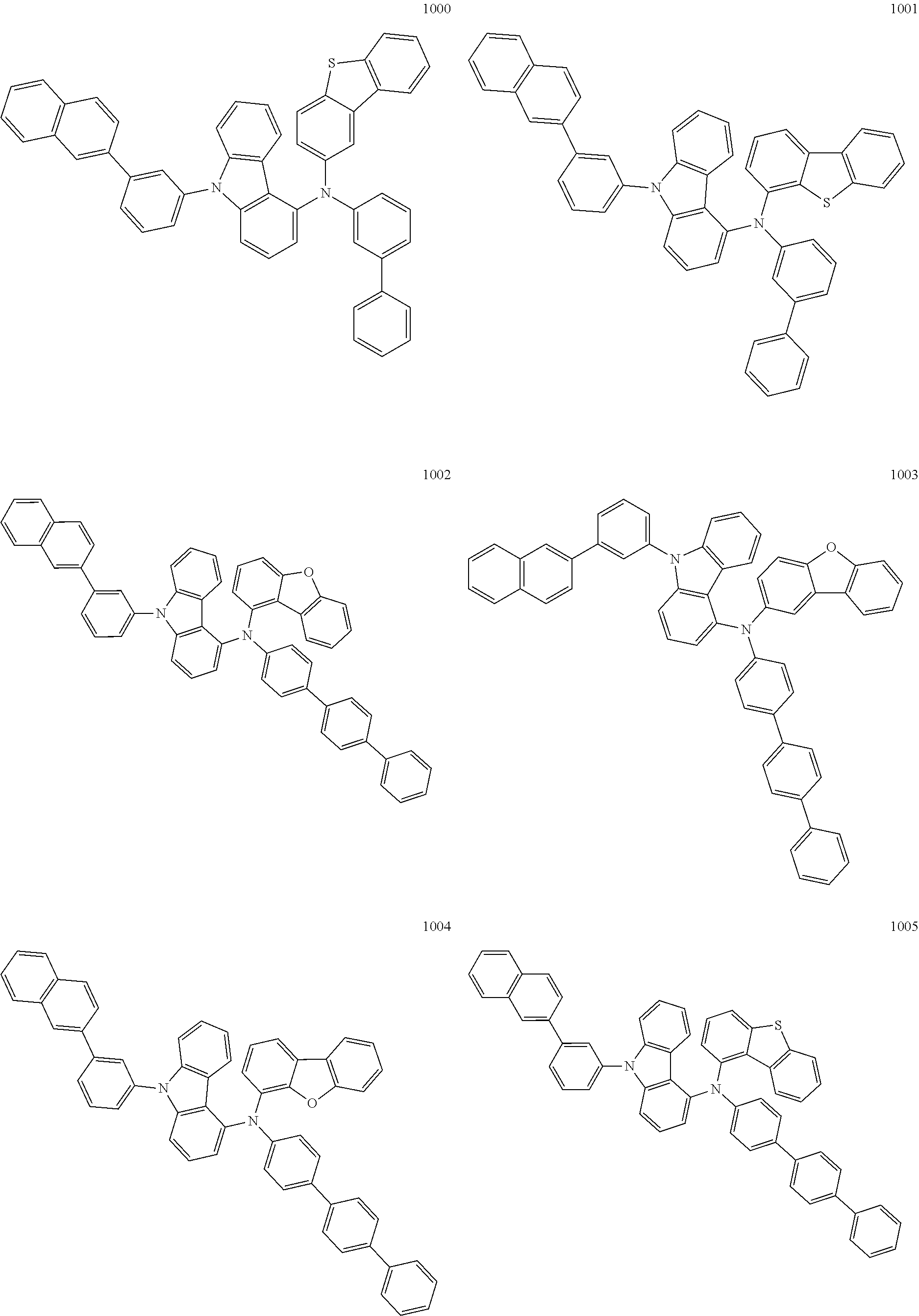 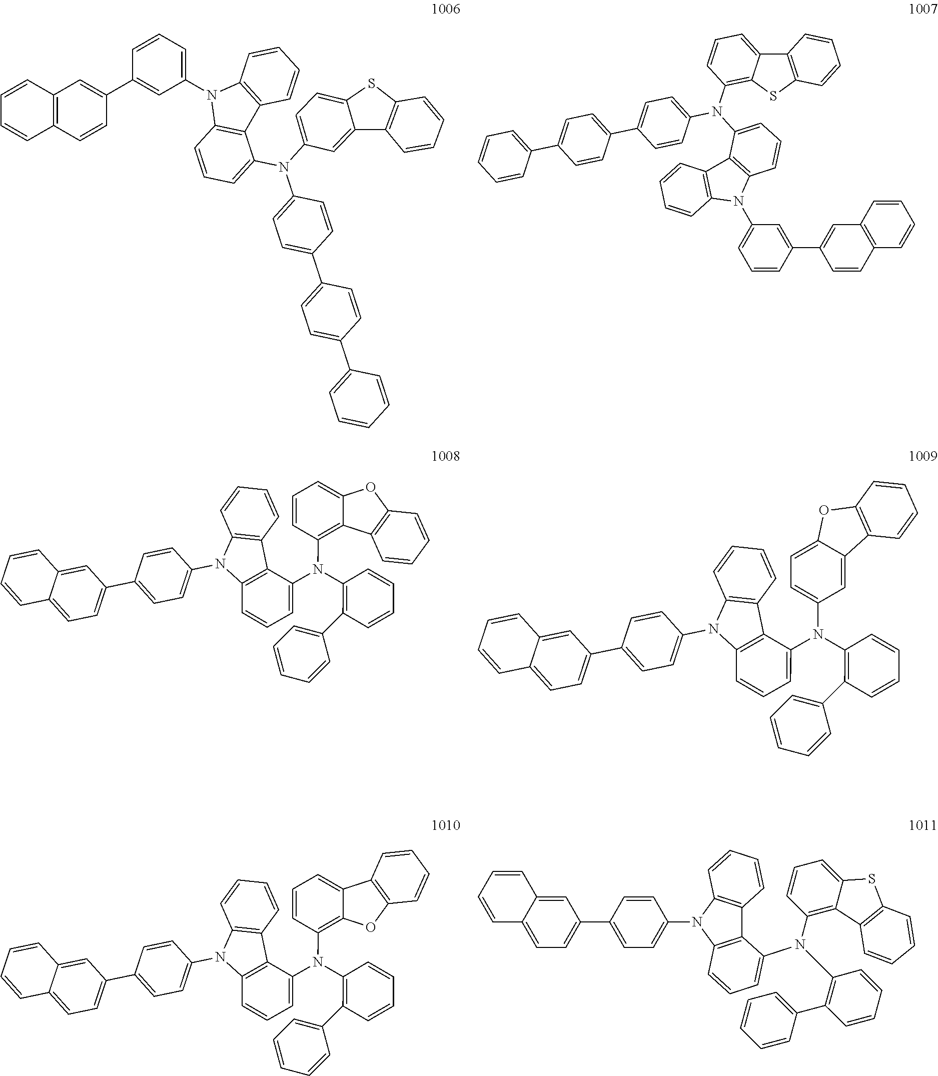 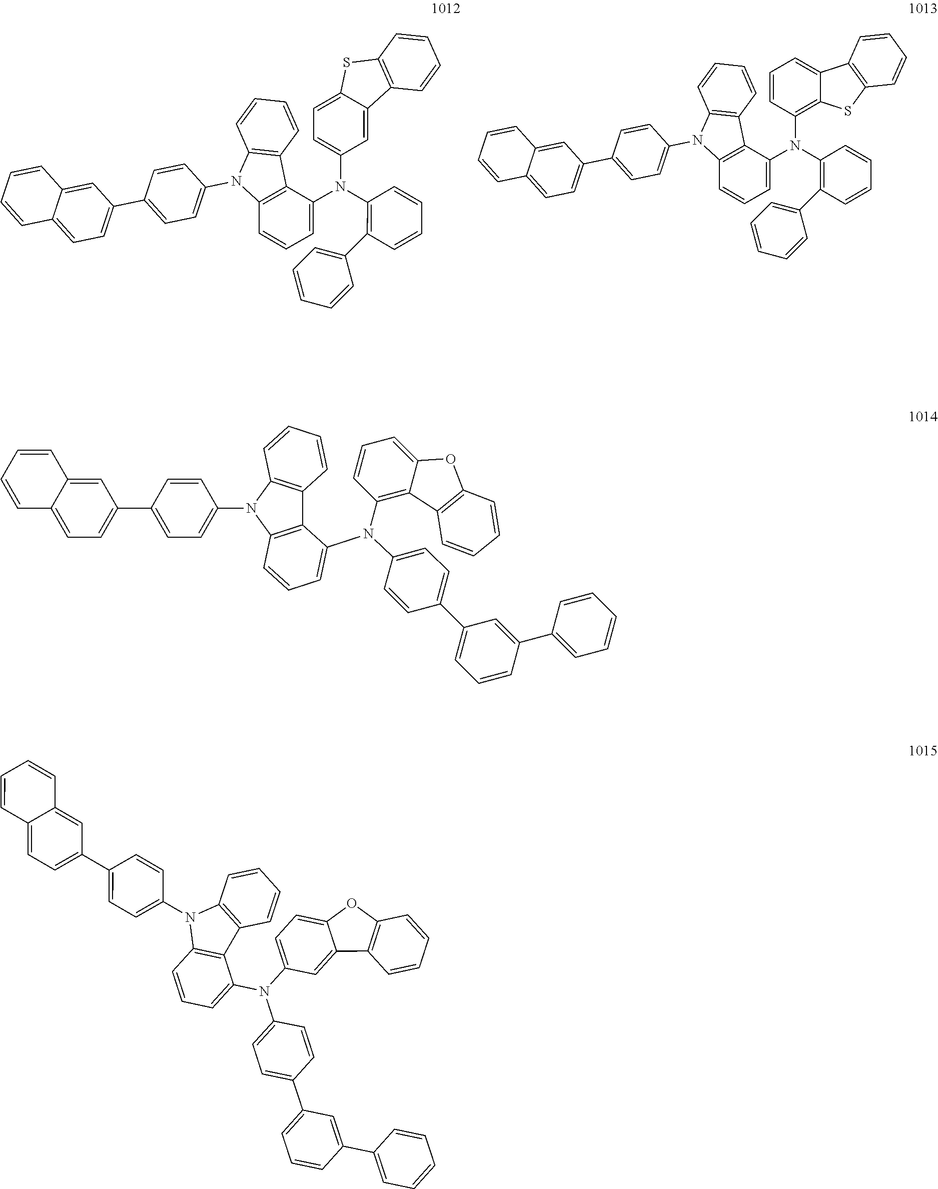 [SMILES: c1ccc(-c2ccccc2N(c2ccc3sc4ccccc4c3c2)c2cccc3c2c2ccccc2n3-c2ccc(-c3ccc4ccccc4c3)cc2)cc1 ; c1ccc(-c2ccccc2N(c2cccc3c2sc2ccccc23)c2cccc3c2c2ccccc2n3-c2ccc(-c3ccc4ccccc4c3)cc2)cc1 ; c1ccc(-c2cccc(-c3ccc(N(c4cccc5oc6ccccc6c45)c4cccc5c4c4ccccc4n5-c4ccc(-c5ccc6ccccc6c5)cc4)cc3)c2)cc1 ; c1ccc(-c2cccc(-c3ccc(N(c4ccc5oc6ccccc6c5c4)c4cccc5c4c4ccccc4n5-c4ccc(-c5ccc6ccccc6c5)cc4)cc3)c2)cc1] 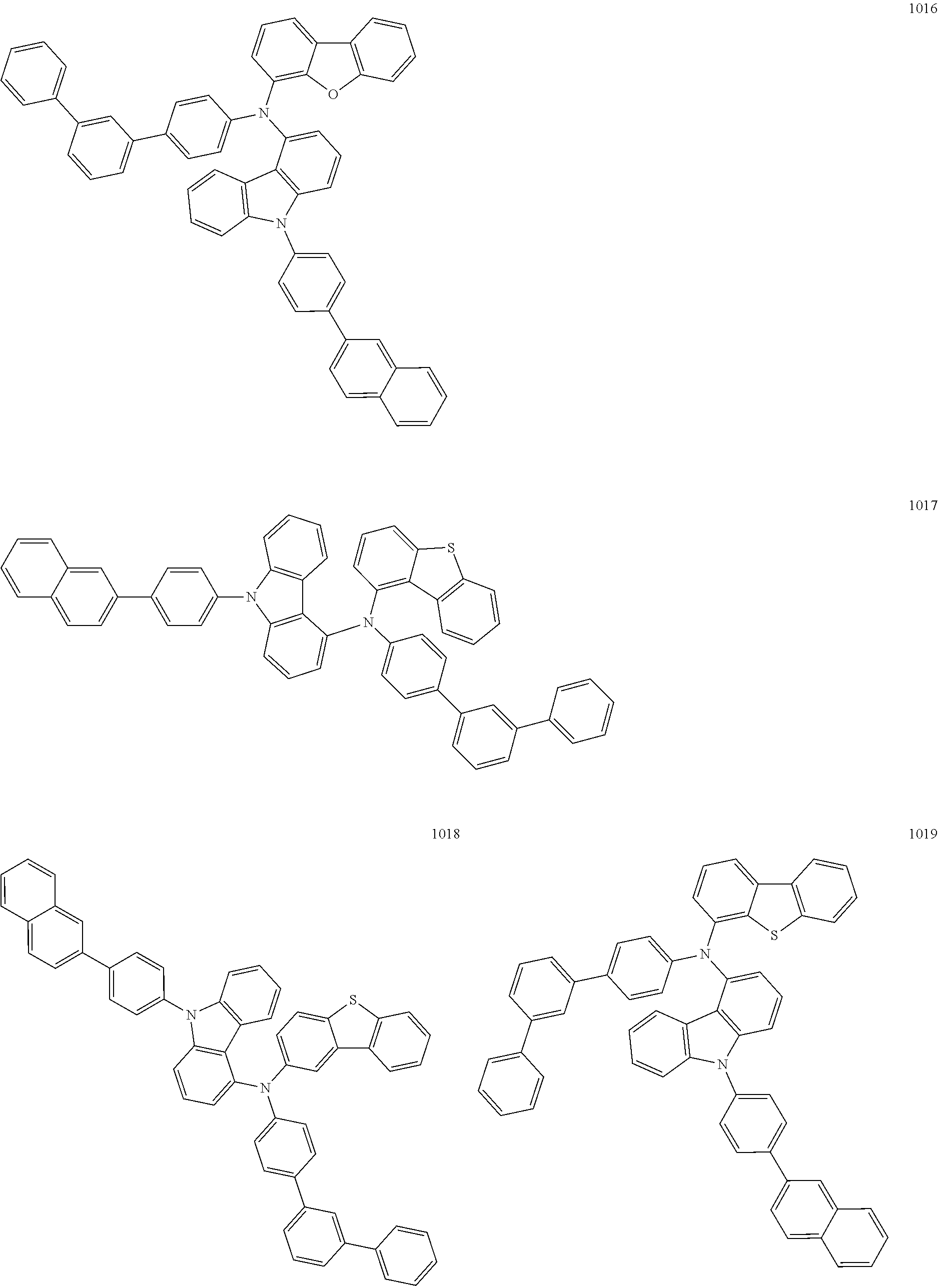 [SMILES: c1ccc(-c2cccc(-c3ccc(N(c4cccc5c4oc4ccccc45)c4cccc5c4c4ccccc4n5-c4ccc(-c5ccc6ccccc6c5)cc4)cc3)c2)cc1 ; c1ccc(-c2cccc(-c3ccc(N(c4cccc5sc6ccccc6c45)c4cccc5c4c4ccccc4n5-c4ccc(-c5ccc6ccccc6c5)cc4)cc3)c2)cc1 ; c1ccc(-c2cccc(-c3ccc(N(c4ccc5sc6ccccc6c5c4)c4cccc5c4c4ccccc4n5-c4ccc(-c5ccc6ccccc6c5)cc4)cc3)c2)cc1 ; c1ccc(-c2cccc(-c3ccc(N(c4cccc5c4sc4ccccc45)c4cccc5c4c4ccccc4n5-c4ccc(-c5ccc6ccccc6c5)cc4)cc3)c2)cc1] 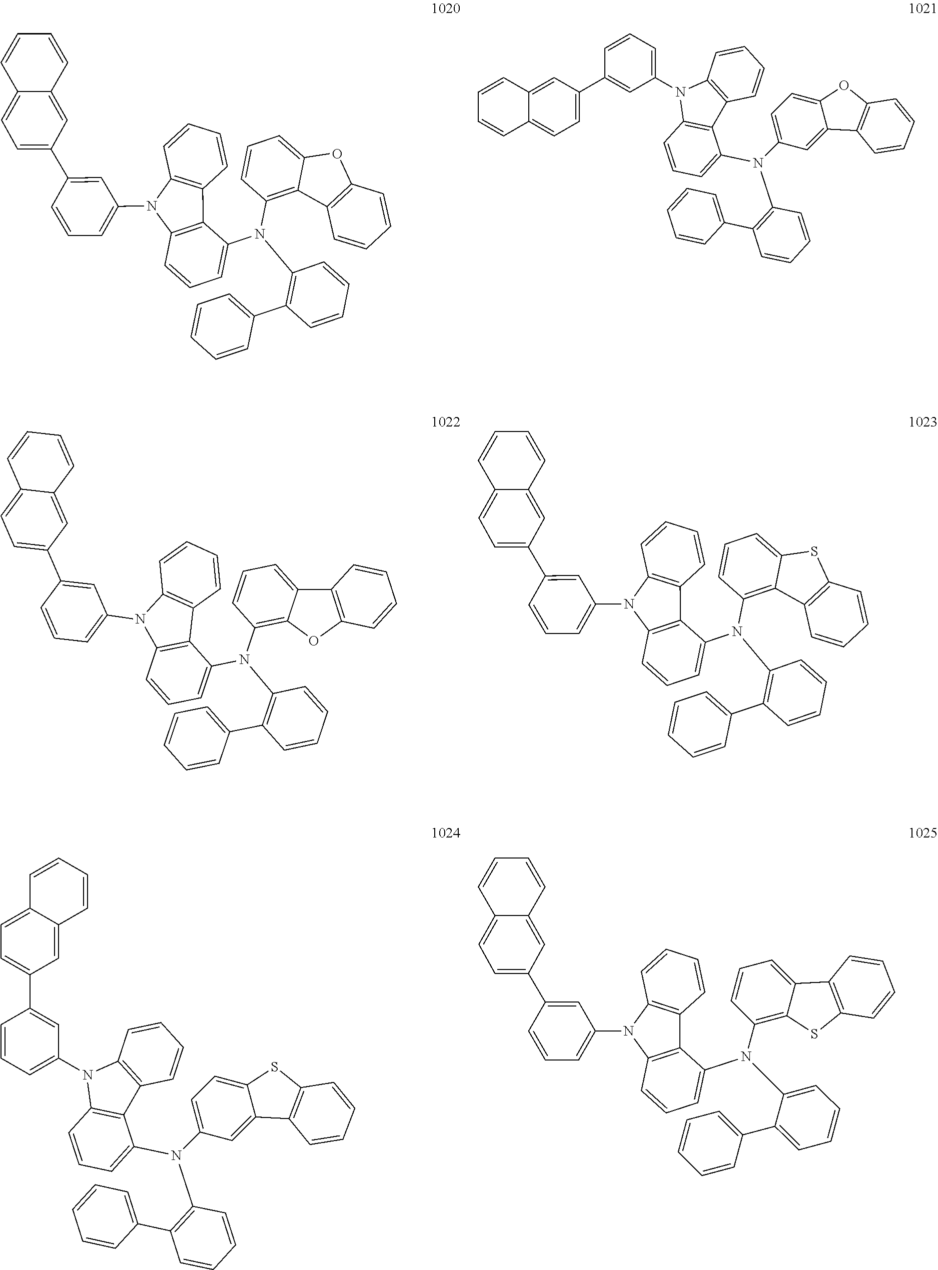 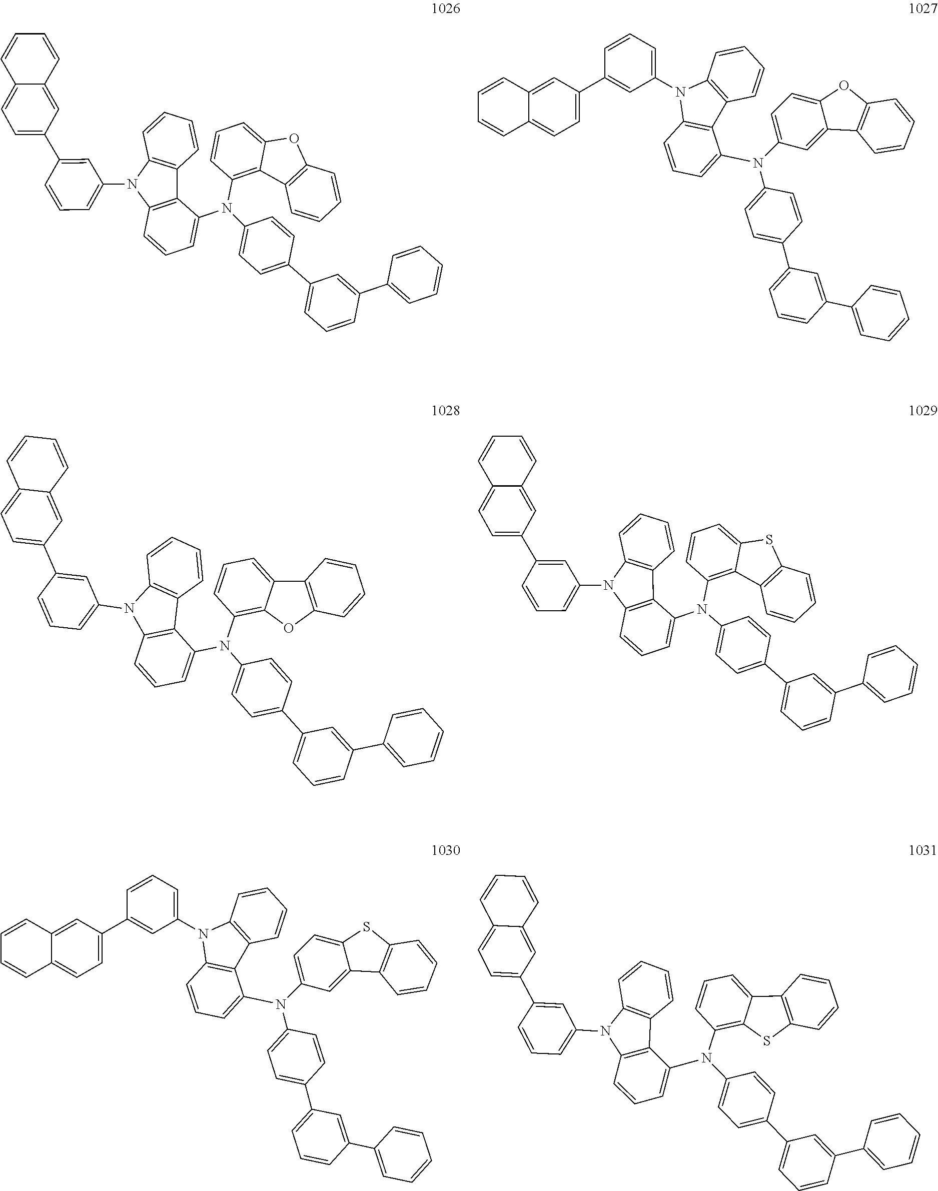 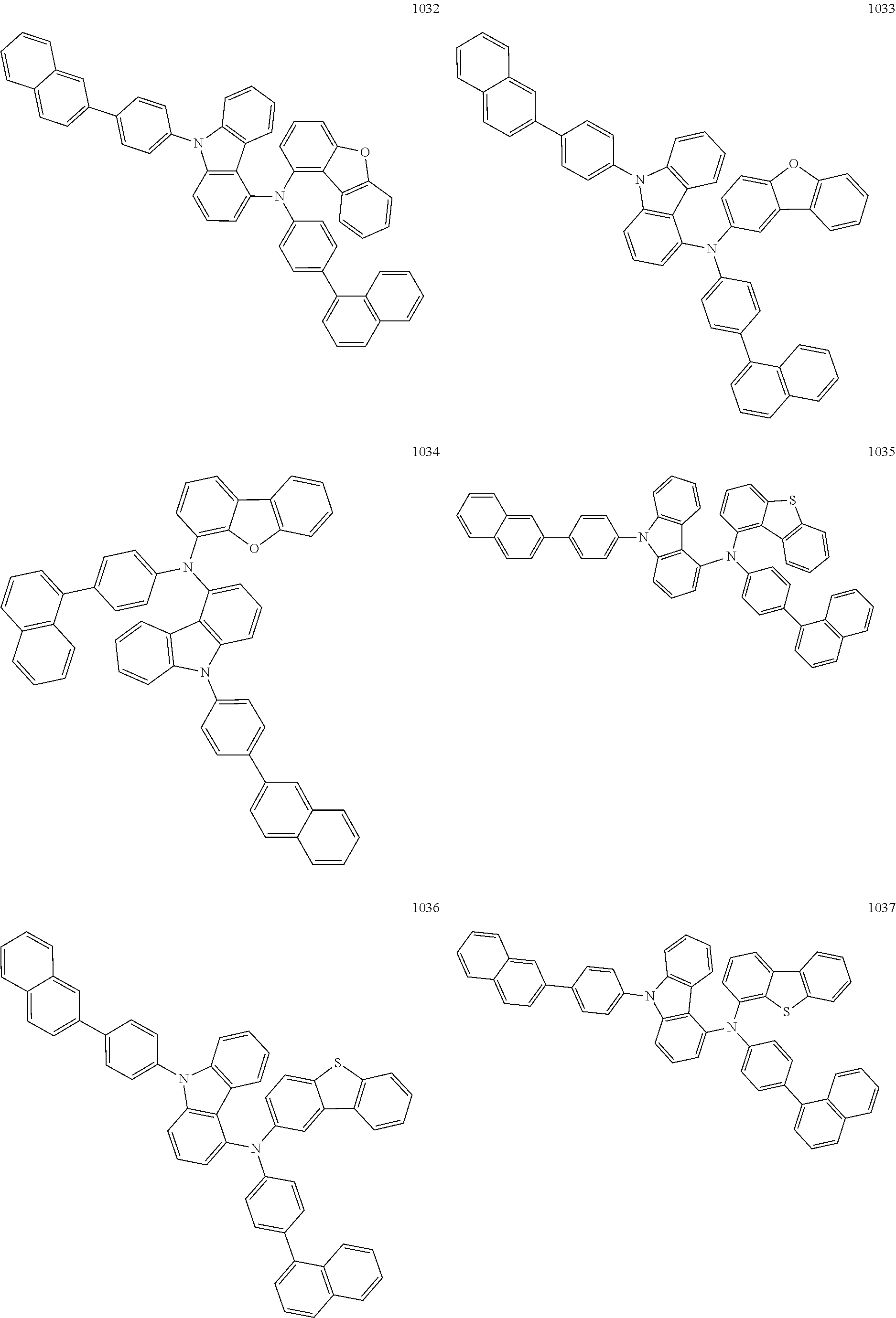 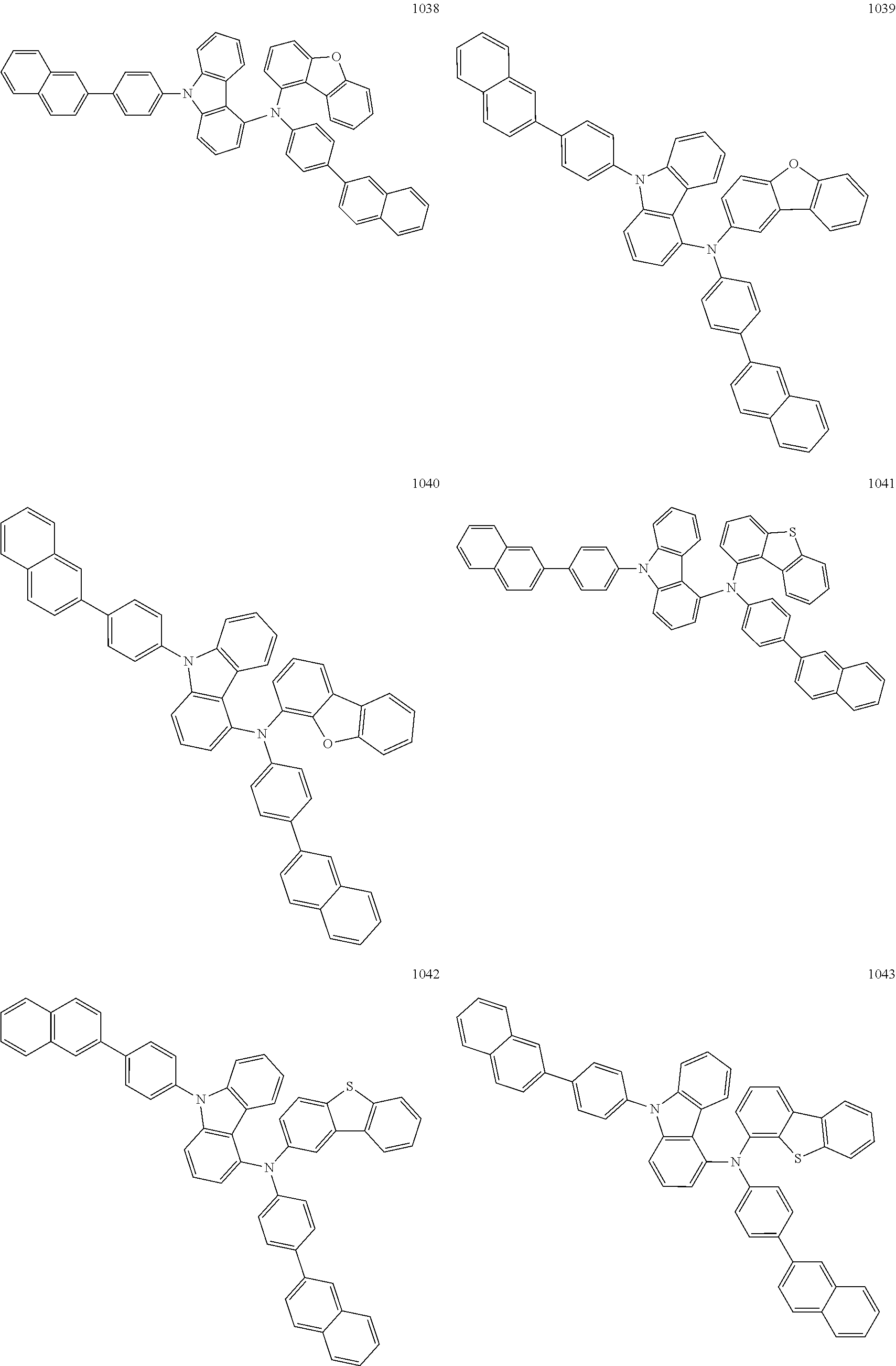 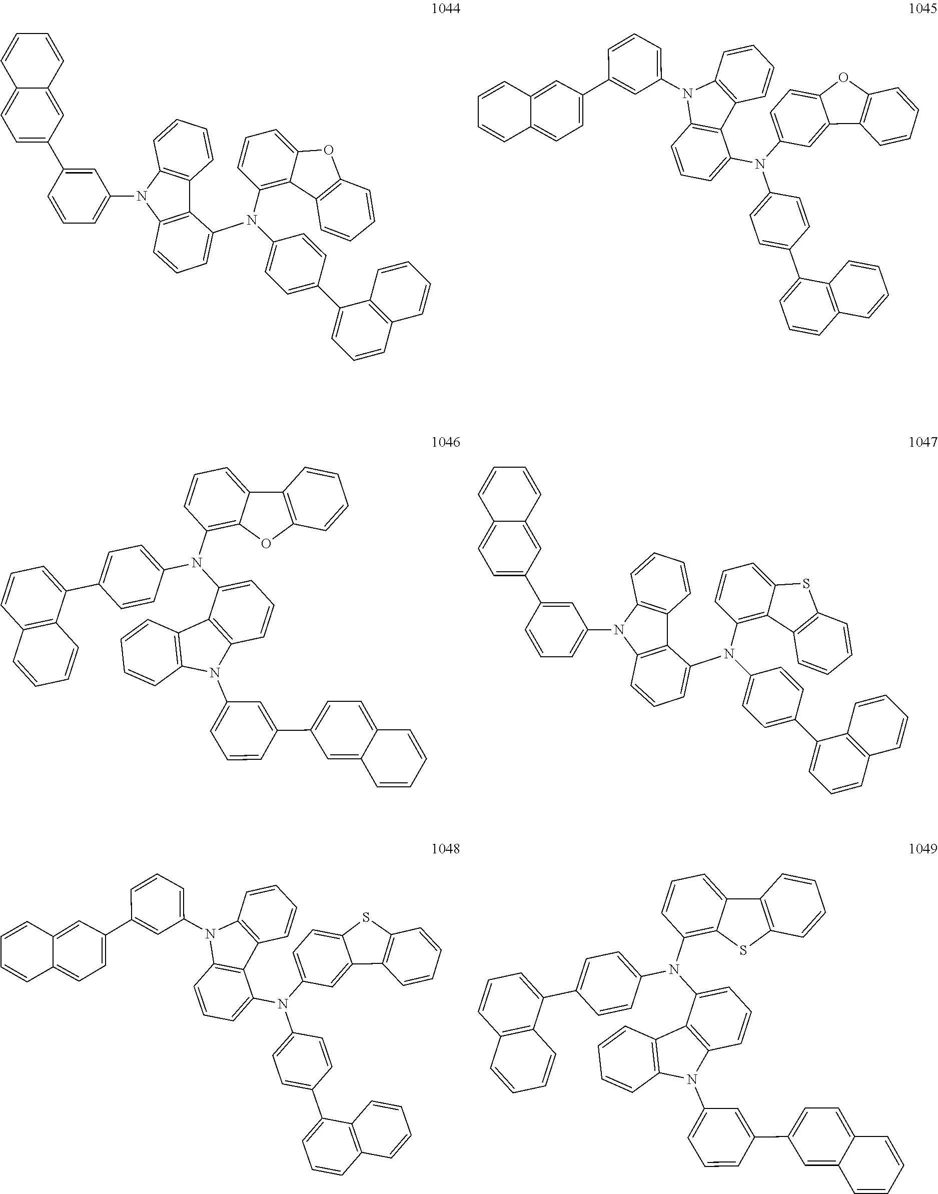 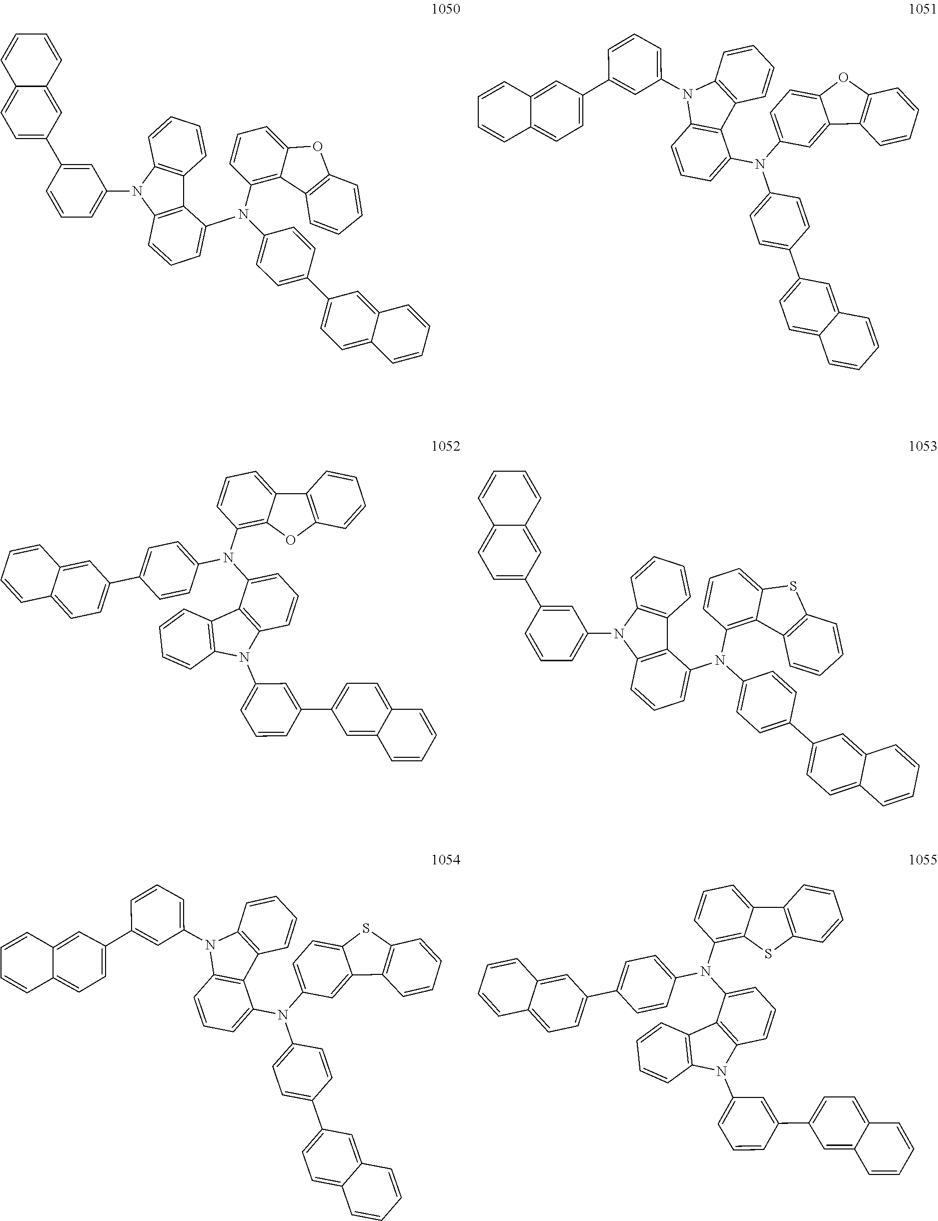 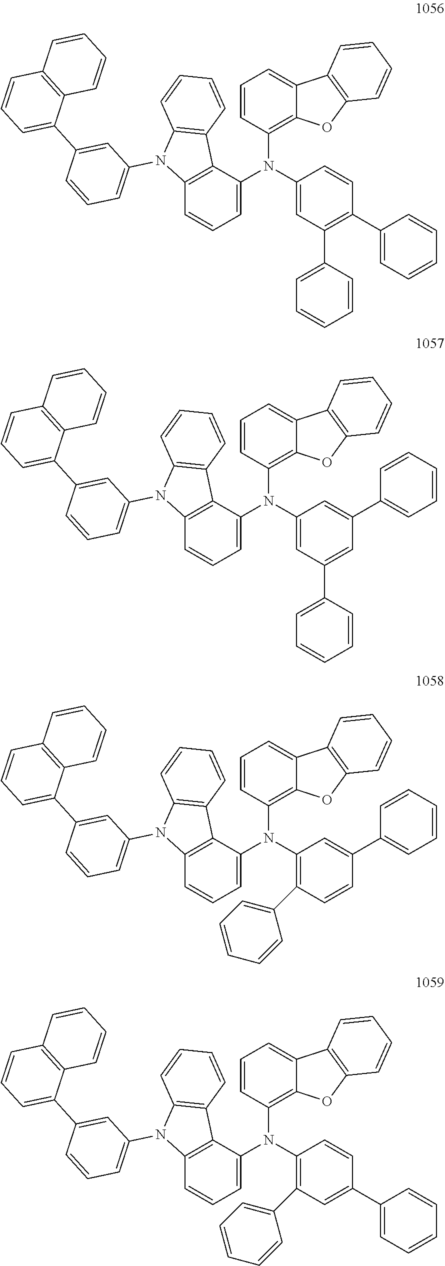 [SMILES: c1ccc(-c2ccc(N(c3cccc4c3oc3ccccc34)c3cccc4c3c3ccccc3n4-c3cccc(-c4cccc5ccccc45)c3)cc2-c2ccccc2)cc1 ; c1ccc(-c2cc(-c3ccccc3)cc(N(c3cccc4c3oc3ccccc34)c3cccc4c3c3ccccc3n4-c3cccc(-c4cccc5ccccc45)c3)c2)cc1 ; c1ccc(-c2ccc(-c3ccccc3)c(N(c3cccc4c3oc3ccccc34)c3cccc4c3c3ccccc3n4-c3cccc(-c4cccc5ccccc45)c3)c2)cc1 ; c1ccc(-c2ccc(N(c3cccc4c3oc3ccccc34)c3cccc4c3c3ccccc3n4-c3cccc(-c4cccc5ccccc45)c3)c(-c3ccccc3)c2)cc1] 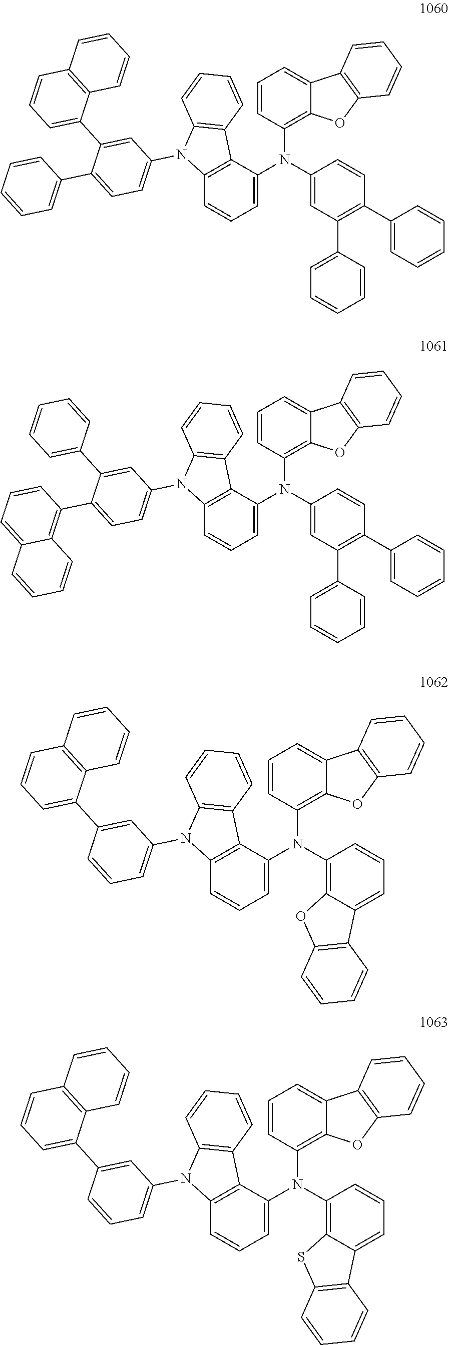 [SMILES: c1ccc(-c2ccc(N(c3cccc4c3oc3ccccc34)c3cccc4c3c3ccccc3n4-c3ccc(-c4ccccc4)c(-c4cccc5ccccc45)c3)cc2-c2ccccc2)cc1 ; c1ccc(-c2ccc(N(c3cccc4c3oc3ccccc34)c3cccc4c3c3ccccc3n4-c3ccc(-c4cccc5ccccc45)c(-c4ccccc4)c3)cc2-c2ccccc2)cc1 ; c1cc(-c2cccc3ccccc23)cc(-n2c3ccccc3c3c(N(c4cccc5c4oc4ccccc45)c4cccc5c4oc4ccccc45)cccc32)c1 ; c1cc(-c2cccc3ccccc23)cc(-n2c3ccccc3c3c(N(c4cccc5c4oc4ccccc45)c4cccc5c4sc4ccccc45)cccc32)c1] 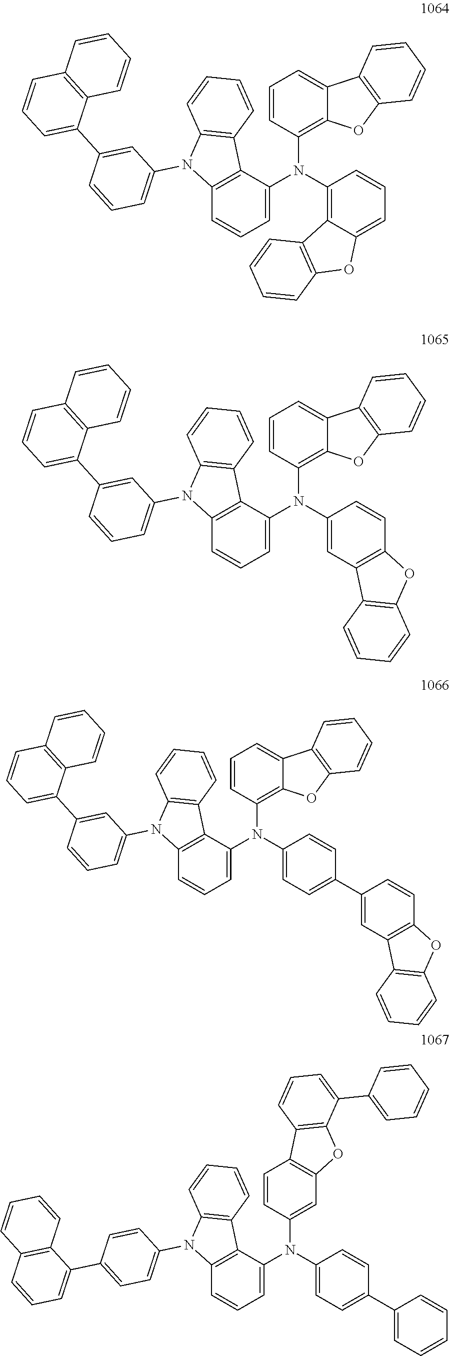 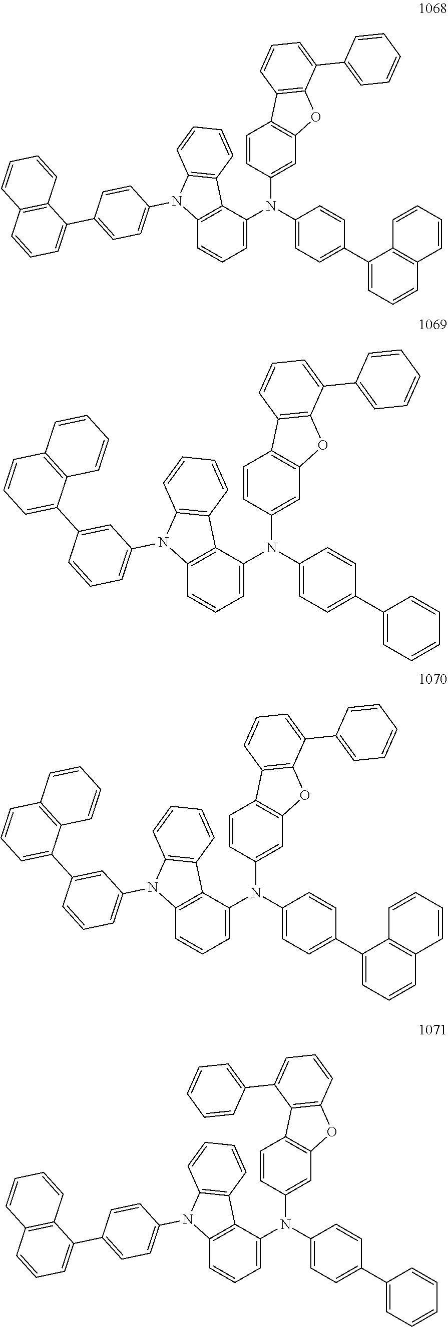 [SMILES: c1ccc(-c2cccc3c2oc2cc(N(c4ccc(-c5cccc6ccccc56)cc4)c4cccc5c4c4ccccc4n5-c4ccc(-c5cccc6ccccc56)cc4)ccc23)cc1 ; c1ccc(-c2ccc(N(c3ccc4c(c3)oc3c(-c5ccccc5)cccc34)c3cccc4c3c3ccccc3n4-c3cccc(-c4cccc5ccccc45)c3)cc2)cc1 ; c1ccc(-c2cccc3c2oc2cc(N(c4ccc(-c5cccc6ccccc56)cc4)c4cccc5c4c4ccccc4n5-c4cccc(-c5cccc6ccccc56)c4)ccc23)cc1 ; c1ccc(-c2ccc(N(c3ccc4c(c3)oc3cccc(-c5ccccc5)c34)c3cccc4c3c3ccccc3n4-c3ccc(-c4cccc5ccccc45)cc3)cc2)cc1] 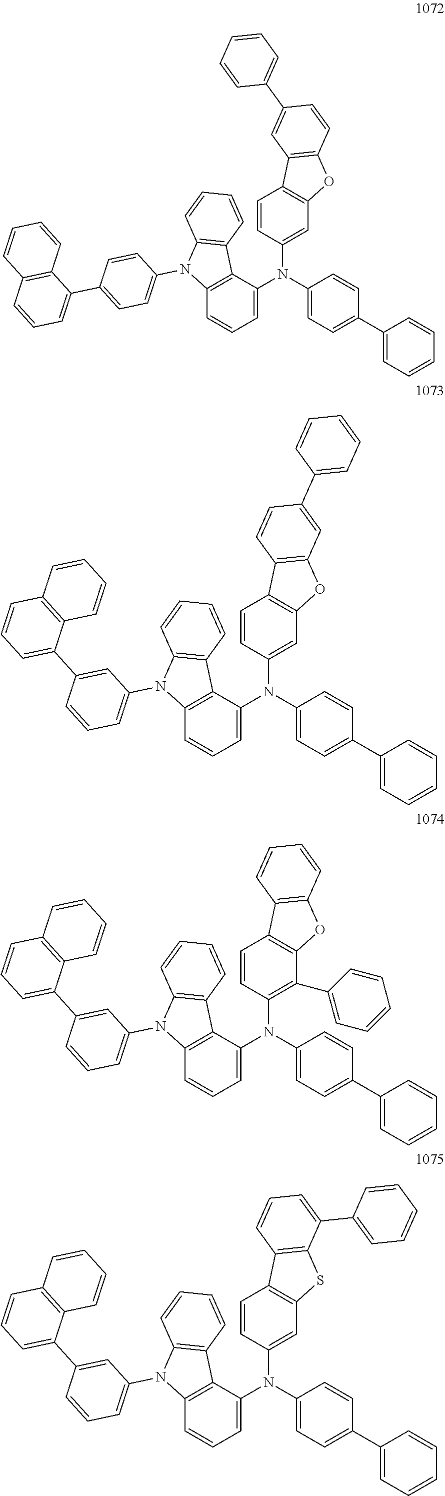 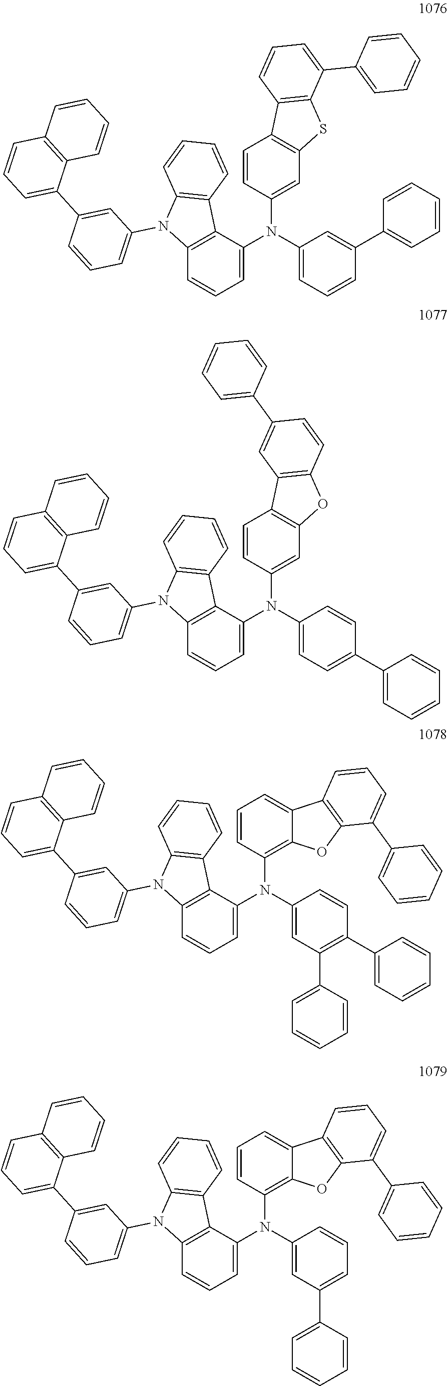 [SMILES: c1ccc(-c2cccc(N(c3ccc4c(c3)sc3c(-c5ccccc5)cccc34)c3cccc4c3c3ccccc3n4-c3cccc(-c4cccc5ccccc45)c3)c2)cc1 ; c1ccc(-c2ccc(N(c3ccc4c(c3)oc3ccc(-c5ccccc5)cc34)c3cccc4c3c3ccccc3n4-c3cccc(-c4cccc5ccccc45)c3)cc2)cc1 ; c1ccc(-c2ccc(N(c3cccc4c3oc3c(-c5ccccc5)cccc34)c3cccc4c3c3ccccc3n4-c3cccc(-c4cccc5ccccc45)c3)cc2-c2ccccc2)cc1 ; c1ccc(-c2cccc(N(c3cccc4c3oc3c(-c5ccccc5)cccc34)c3cccc4c3c3ccccc3n4-c3cccc(-c4cccc5ccccc45)c3)c2)cc1] 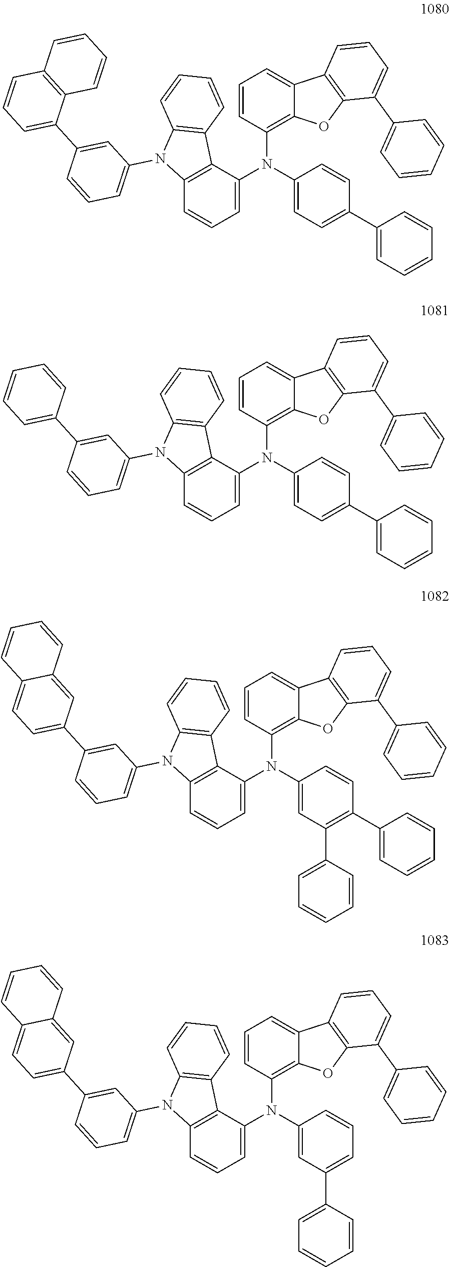 [SMILES: c1ccc(-c2ccc(N(c3cccc4c3oc3c(-c5ccccc5)cccc34)c3cccc4c3c3ccccc3n4-c3cccc(-c4cccc5ccccc45)c3)cc2)cc1 ; c1ccc(-c2ccc(N(c3cccc4c3oc3c(-c5ccccc5)cccc34)c3cccc4c3c3ccccc3n4-c3cccc(-c4ccccc4)c3)cc2)cc1 ; c1ccc(-c2ccc(N(c3cccc4c3oc3c(-c5ccccc5)cccc34)c3cccc4c3c3ccccc3n4-c3cccc(-c4ccc5ccccc5c4)c3)cc2-c2ccccc2)cc1 ; c1ccc(-c2cccc(N(c3cccc4c3oc3c(-c5ccccc5)cccc34)c3cccc4c3c3ccccc3n4-c3cccc(-c4ccc5ccccc5c4)c3)c2)cc1] 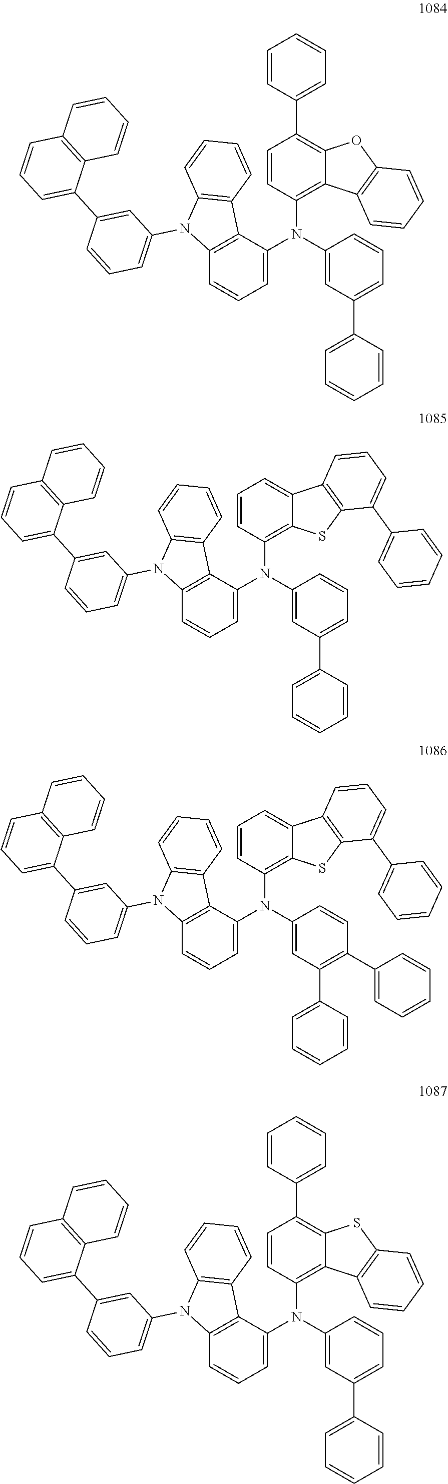 [SMILES: c1ccc(-c2cccc(N(c3ccc(-c4ccccc4)c4oc5ccccc5c34)c3cccc4c3c3ccccc3n4-c3cccc(-c4cccc5ccccc45)c3)c2)cc1 ; c1ccc(-c2cccc(N(c3cccc4c3sc3c(-c5ccccc5)cccc34)c3cccc4c3c3ccccc3n4-c3cccc(-c4cccc5ccccc45)c3)c2)cc1 ; c1ccc(-c2ccc(N(c3cccc4c3sc3c(-c5ccccc5)cccc34)c3cccc4c3c3ccccc3n4-c3cccc(-c4cccc5ccccc45)c3)cc2-c2ccccc2)cc1 ; c1ccc(-c2cccc(N(c3ccc(-c4ccccc4)c4sc5ccccc5c34)c3cccc4c3c3ccccc3n4-c3cccc(-c4cccc5ccccc45)c3)c2)cc1] 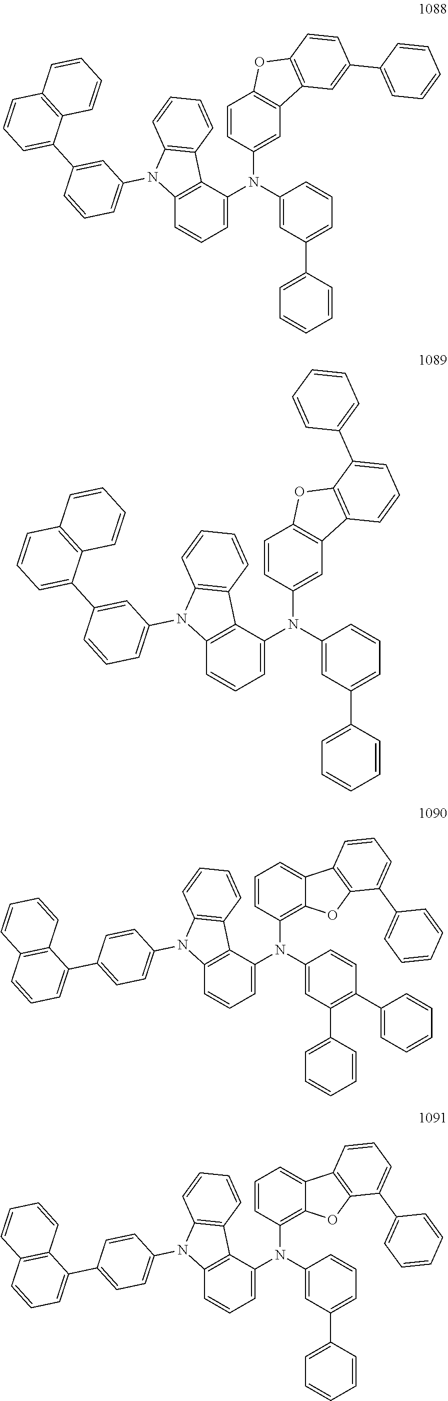 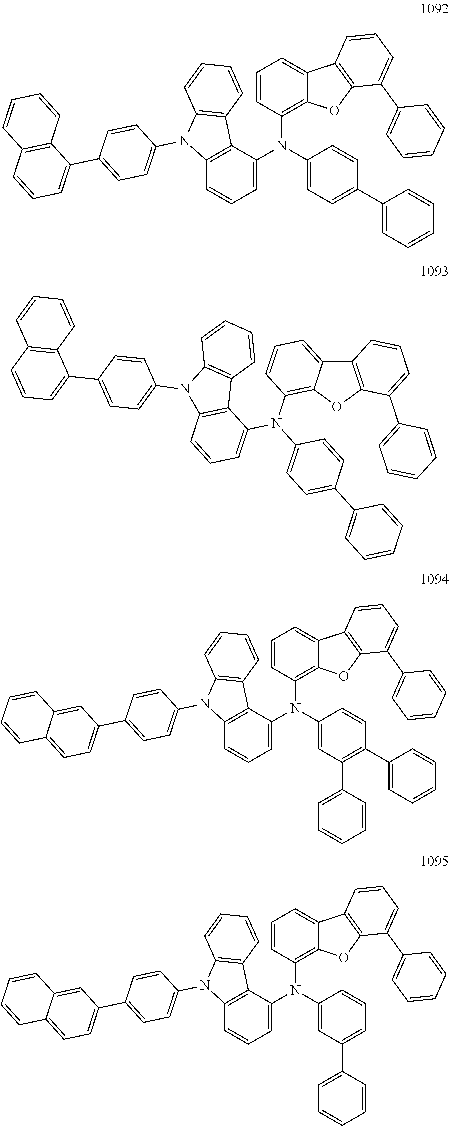 [SMILES: c1ccc(-c2ccc(N(c3cccc4c3oc3c(-c5ccccc5)cccc34)c3cccc4c3c3ccccc3n4-c3ccc(-c4cccc5ccccc45)cc3)cc2)cc1 ; c1ccc(-c2ccc(N(c3cccc4c3oc3c(-c5ccccc5)cccc34)c3cccc4c3c3ccccc3n4-c3ccc(-c4cccc5ccccc45)cc3)cc2)cc1 ; c1ccc(-c2ccc(N(c3cccc4c3oc3c(-c5ccccc5)cccc34)c3cccc4c3c3ccccc3n4-c3ccc(-c4ccc5ccccc5c4)cc3)cc2-c2ccccc2)cc1 ; c1ccc(-c2cccc(N(c3cccc4c3oc3c(-c5ccccc5)cccc34)c3cccc4c3c3ccccc3n4-c3ccc(-c4ccc5ccccc5c4)cc3)c2)cc1] 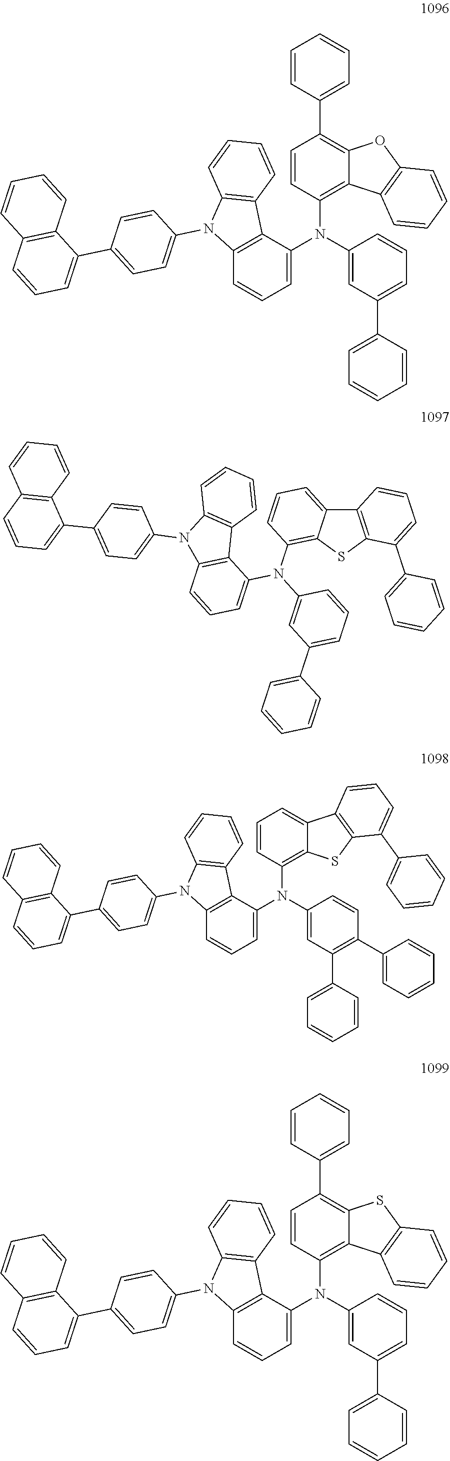 [SMILES: c1ccc(-c2cccc(N(c3ccc(-c4ccccc4)c4oc5ccccc5c34)c3cccc4c3c3ccccc3n4-c3ccc(-c4cccc5ccccc45)cc3)c2)cc1 ; c1ccc(-c2cccc(N(c3cccc4c3sc3c(-c5ccccc5)cccc34)c3cccc4c3c3ccccc3n4-c3ccc(-c4cccc5ccccc45)cc3)c2)cc1 ; c1ccc(-c2ccc(N(c3cccc4c3sc3c(-c5ccccc5)cccc34)c3cccc4c3c3ccccc3n4-c3ccc(-c4cccc5ccccc45)cc3)cc2-c2ccccc2)cc1 ; c1ccc(-c2cccc(N(c3ccc(-c4ccccc4)c4sc5ccccc5c34)c3cccc4c3c3ccccc3n4-c3ccc(-c4cccc5ccccc45)cc3)c2)cc1] 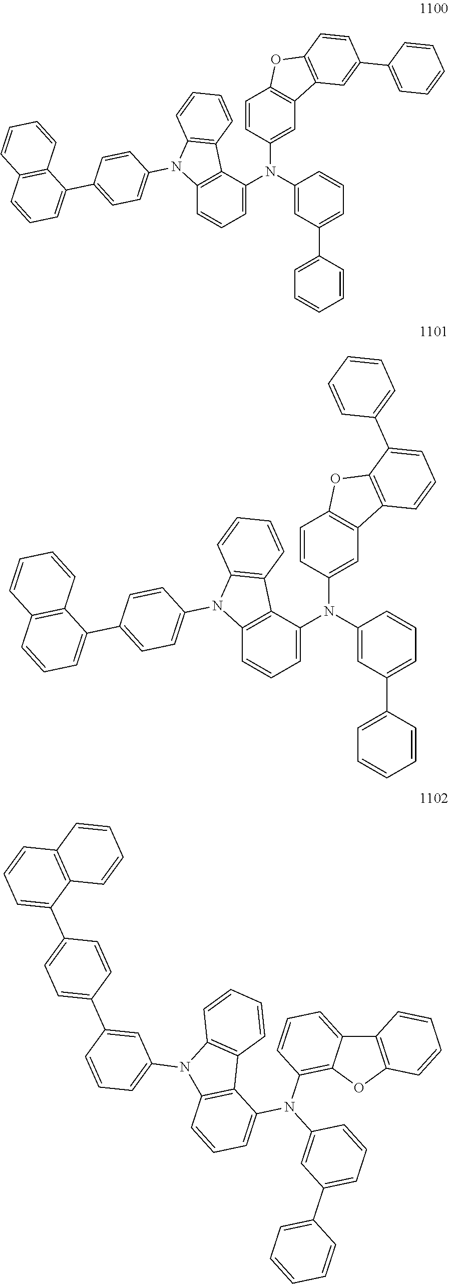 [SMILES: c1ccc(-c2cccc(N(c3ccc4oc5ccc(-c6ccccc6)cc5c4c3)c3cccc4c3c3ccccc3n4-c3ccc(-c4cccc5ccccc45)cc3)c2)cc1 ; c1ccc(-c2cccc(N(c3ccc4oc5c(-c6ccccc6)cccc5c4c3)c3cccc4c3c3ccccc3n4-c3ccc(-c4cccc5ccccc45)cc3)c2)cc1 ; c1ccc(-c2cccc(N(c3cccc4c3oc3ccccc34)c3cccc4c3c3ccccc3n4-c3cccc(-c4ccc(-c5cccc6ccccc56)cc4)c3)c2)cc1] 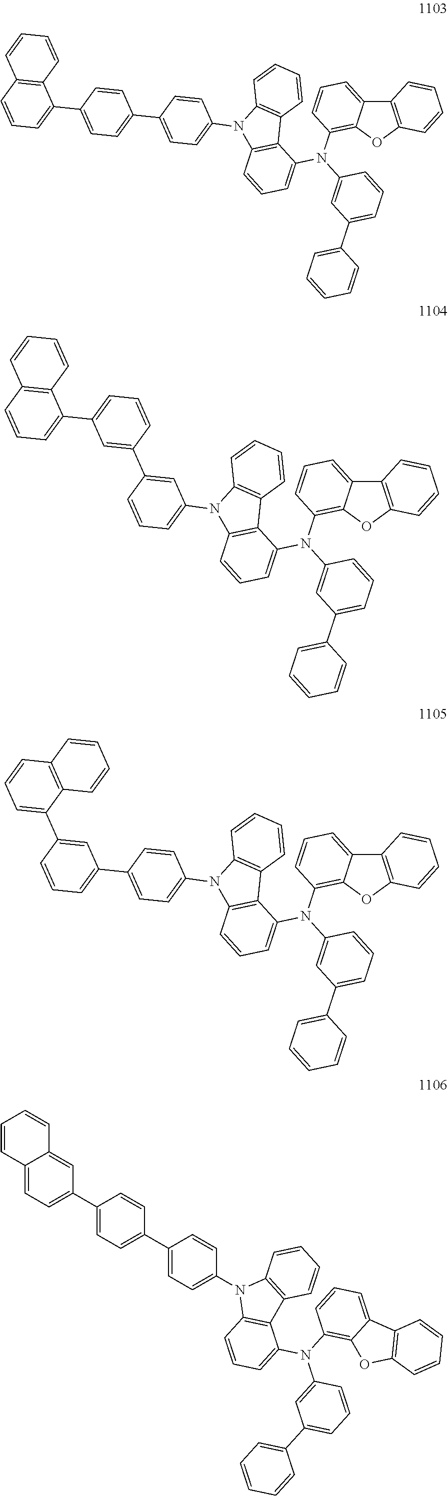 [SMILES: c1ccc(-c2cccc(N(c3cccc4c3oc3ccccc34)c3cccc4c3c3ccccc3n4-c3ccc(-c4ccc(-c5cccc6ccccc56)cc4)cc3)c2)cc1 ; c1ccc(-c2cccc(N(c3cccc4c3oc3ccccc34)c3cccc4c3c3ccccc3n4-c3cccc(-c4cccc(-c5cccc6ccccc56)c4)c3)c2)cc1 ; c1ccc(-c2cccc(N(c3cccc4c3oc3ccccc34)c3cccc4c3c3ccccc3n4-c3ccc(-c4cccc(-c5cccc6ccccc56)c4)cc3)c2)cc1 ; c1ccc(-c2cccc(N(c3cccc4c3oc3ccccc34)c3cccc4c3c3ccccc3n4-c3ccc(-c4ccc(-c5ccc6ccccc6c5)cc4)cc3)c2)cc1] 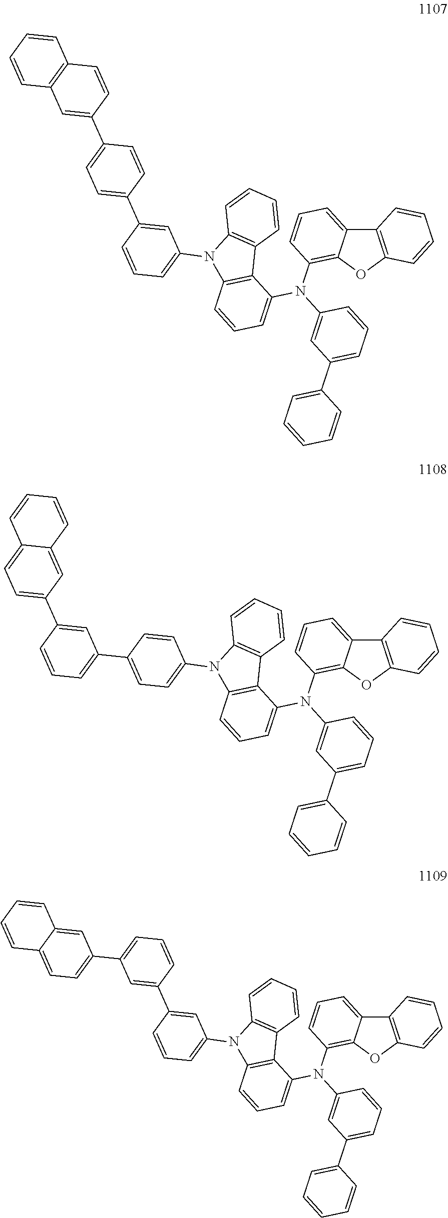 [SMILES: c1ccc(-c2cccc(N(c3cccc4c3oc3ccccc34)c3cccc4c3c3ccccc3n4-c3cccc(-c4ccc(-c5ccc6ccccc6c5)cc4)c3)c2)cc1 ; c1ccc(-c2cccc(N(c3cccc4c3oc3ccccc34)c3cccc4c3c3ccccc3n4-c3ccc(-c4cccc(-c5ccc6ccccc6c5)c4)cc3)c2)cc1 ; c1ccc(-c2cccc(N(c3cccc4c3oc3ccccc34)c3cccc4c3c3ccccc3n4-c3cccc(-c4cccc(-c5ccc6ccccc6c5)c4)c3)c2)cc1] 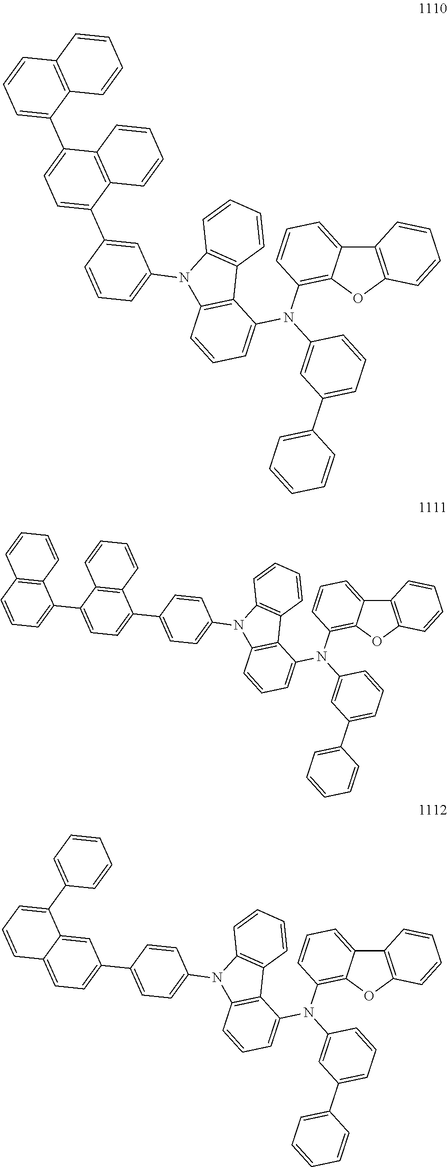 [SMILES: c1ccc(-c2cccc(N(c3cccc4c3oc3ccccc34)c3cccc4c3c3ccccc3n4-c3cccc(-c4ccc(-c5cccc6ccccc56)c5ccccc45)c3)c2)cc1 ; c1ccc(-c2cccc(N(c3cccc4c3oc3ccccc34)c3cccc4c3c3ccccc3n4-c3ccc(-c4ccc(-c5cccc6ccccc56)c5ccccc45)cc3)c2)cc1 ; c1ccc(-c2cccc(N(c3cccc4c3oc3ccccc34)c3cccc4c3c3ccccc3n4-c3ccc(-c4ccc5cccc(-c6ccccc6)c5c4)cc3)c2)cc1] 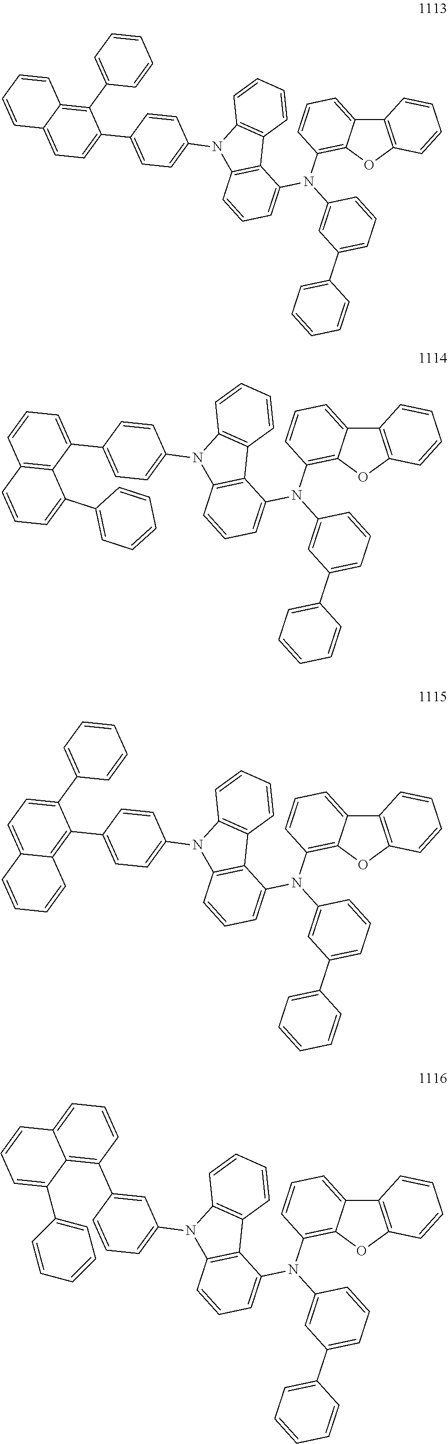 [SMILES: c1ccc(-c2cccc(N(c3cccc4c3oc3ccccc34)c3cccc4c3c3ccccc3n4-c3ccc(-c4ccc5ccccc5c4-c4ccccc4)cc3)c2)cc1 ; c1ccc(-c2cccc(N(c3cccc4c3oc3ccccc34)c3cccc4c3c3ccccc3n4-c3ccc(-c4cccc5cccc(-c6ccccc6)c45)cc3)c2)cc1 ; c1ccc(-c2cccc(N(c3cccc4c3oc3ccccc34)c3cccc4c3c3ccccc3n4-c3ccc(-c4c(-c5ccccc5)ccc5ccccc45)cc3)c2)cc1 ; c1ccc(-c2cccc(N(c3cccc4c3oc3ccccc34)c3cccc4c3c3ccccc3n4-c3cccc(-c4cccc5cccc(-c6ccccc6)c45)c3)c2)cc1] 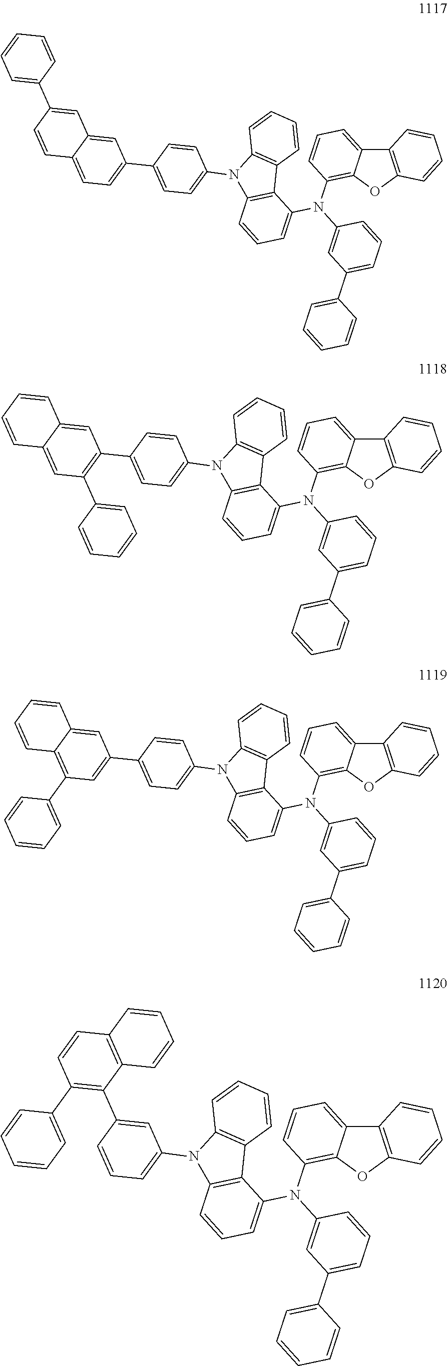 [SMILES: c1ccc(-c2cccc(N(c3cccc4c3oc3ccccc34)c3cccc4c3c3ccccc3n4-c3ccc(-c4ccc5ccc(-c6ccccc6)cc5c4)cc3)c2)cc1 ; c1ccc(-c2cccc(N(c3cccc4c3oc3ccccc34)c3cccc4c3c3ccccc3n4-c3ccc(-c4cc5ccccc5cc4-c4ccccc4)cc3)c2)cc1 ; c1ccc(-c2cccc(N(c3cccc4c3oc3ccccc34)c3cccc4c3c3ccccc3n4-c3ccc(-c4cc(-c5ccccc5)c5ccccc5c4)cc3)c2)cc1 ; c1ccc(-c2cccc(N(c3cccc4c3oc3ccccc34)c3cccc4c3c3ccccc3n4-c3cccc(-c4c(-c5ccccc5)ccc5ccccc45)c3)c2)cc1] 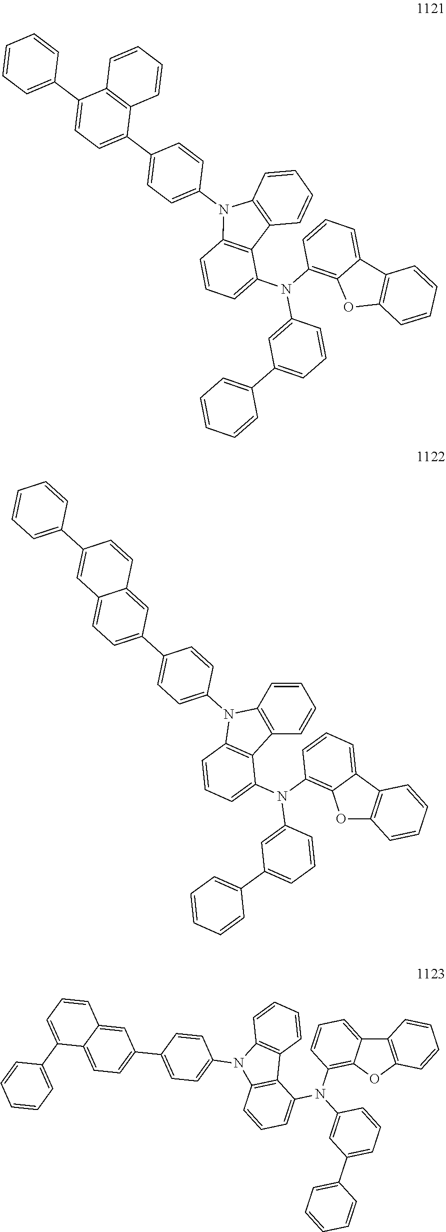 [SMILES: c1ccc(-c2cccc(N(c3cccc4c3oc3ccccc34)c3cccc4c3c3ccccc3n4-c3ccc(-c4ccc(-c5ccccc5)c5ccccc45)cc3)c2)cc1 ; c1ccc(-c2cccc(N(c3cccc4c3oc3ccccc34)c3cccc4c3c3ccccc3n4-c3ccc(-c4ccc5cc(-c6ccccc6)ccc5c4)cc3)c2)cc1 ; c1ccc(-c2cccc(N(c3cccc4c3oc3ccccc34)c3cccc4c3c3ccccc3n4-c3ccc(-c4ccc5c(-c6ccccc6)cccc5c4)cc3)c2)cc1] 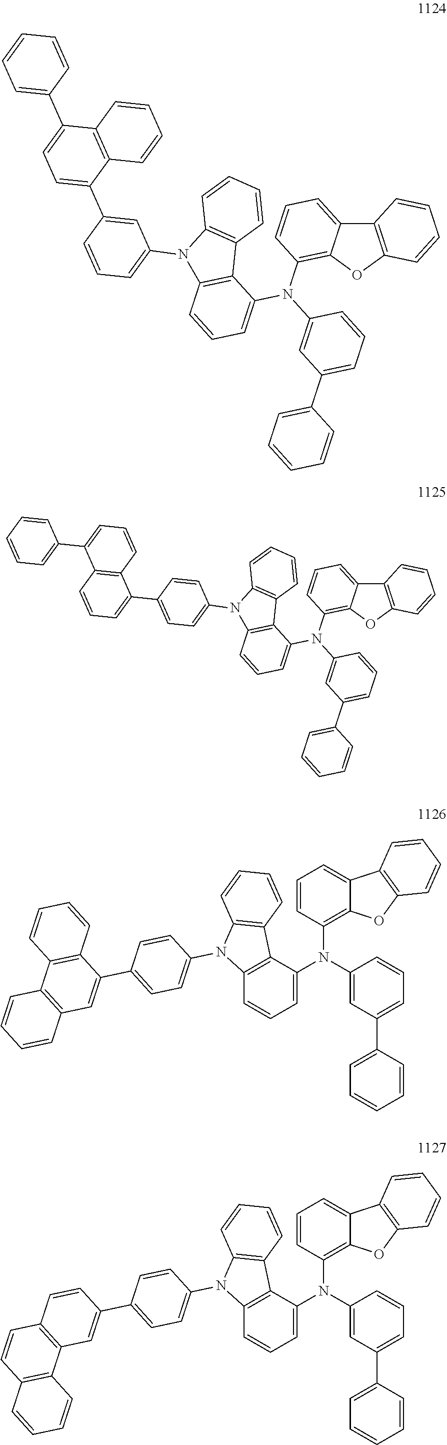 [SMILES: c1ccc(-c2cccc(N(c3cccc4c3oc3ccccc34)c3cccc4c3c3ccccc3n4-c3cccc(-c4ccc(-c5ccccc5)c5ccccc45)c3)c2)cc1 ; c1ccc(-c2cccc(N(c3cccc4c3oc3ccccc34)c3cccc4c3c3ccccc3n4-c3ccc(-c4cccc5c(-c6ccccc6)cccc45)cc3)c2)cc1 ; c1ccc(-c2cccc(N(c3cccc4c3oc3ccccc34)c3cccc4c3c3ccccc3n4-c3ccc(-c4cc5ccccc5c5ccccc45)cc3)c2)cc1 ; c1ccc(-c2cccc(N(c3cccc4c3oc3ccccc34)c3cccc4c3c3ccccc3n4-c3ccc(-c4ccc5ccc6ccccc6c5c4)cc3)c2)cc1] 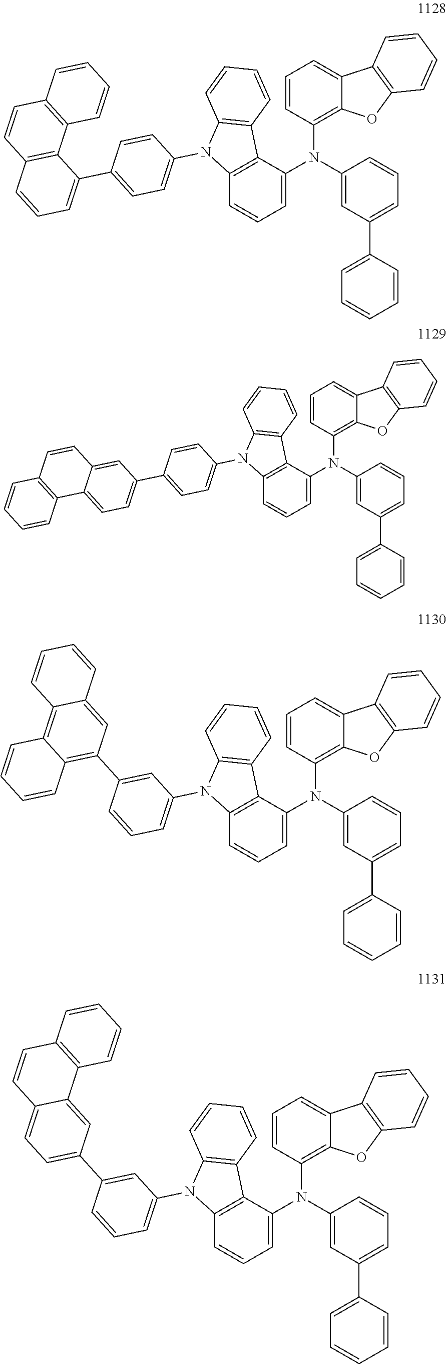 [SMILES: c1ccc(-c2cccc(N(c3cccc4c3oc3ccccc34)c3cccc4c3c3ccccc3n4-c3ccc(-c4cccc5ccc6ccccc6c45)cc3)c2)cc1 ; c1ccc(-c2cccc(N(c3cccc4c3oc3ccccc34)c3cccc4c3c3ccccc3n4-c3ccc(-c4ccc5c(ccc6ccccc65)c4)cc3)c2)cc1 ; c1ccc(-c2cccc(N(c3cccc4c3oc3ccccc34)c3cccc4c3c3ccccc3n4-c3cccc(-c4cc5ccccc5c5ccccc45)c3)c2)cc1 ; c1ccc(-c2cccc(N(c3cccc4c3oc3ccccc34)c3cccc4c3c3ccccc3n4-c3cccc(-c4ccc5ccc6ccccc6c5c4)c3)c2)cc1] 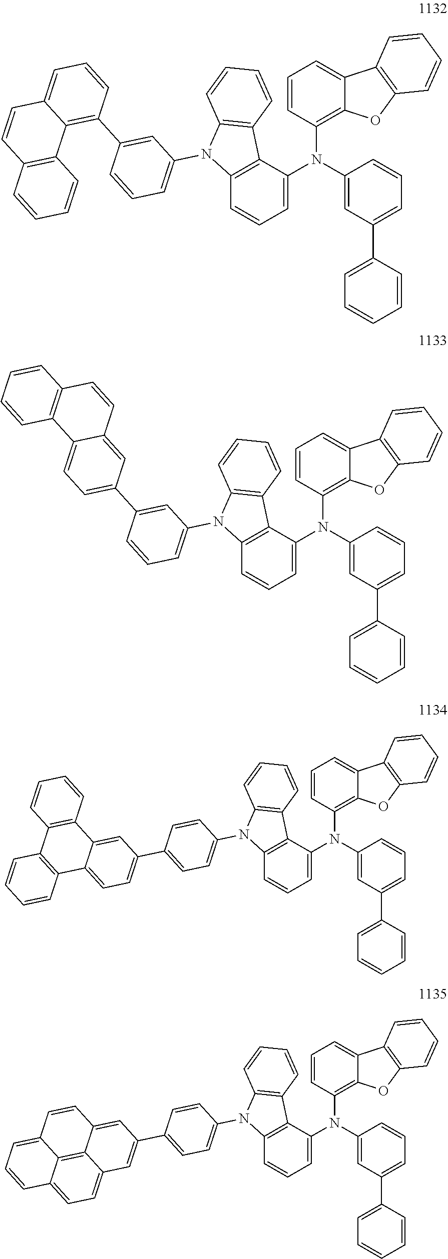 [SMILES: c1ccc(-c2cccc(N(c3cccc4c3oc3ccccc34)c3cccc4c3c3ccccc3n4-c3cccc(-c4cccc5ccc6ccccc6c45)c3)c2)cc1 ; c1ccc(-c2cccc(N(c3cccc4c3oc3ccccc34)c3cccc4c3c3ccccc3n4-c3cccc(-c4ccc5c(ccc6ccccc65)c4)c3)c2)cc1 ; c1ccc(-c2cccc(N(c3cccc4c3oc3ccccc34)c3cccc4c3c3ccccc3n4-c3ccc(-c4ccc5c6ccccc6c6ccccc6c5c4)cc3)c2)cc1 ; c1ccc(-c2cccc(N(c3cccc4c3oc3ccccc34)c3cccc4c3c3ccccc3n4-c3ccc(-c4cc5ccc6cccc7ccc(c4)c5c67)cc3)c2)cc1] 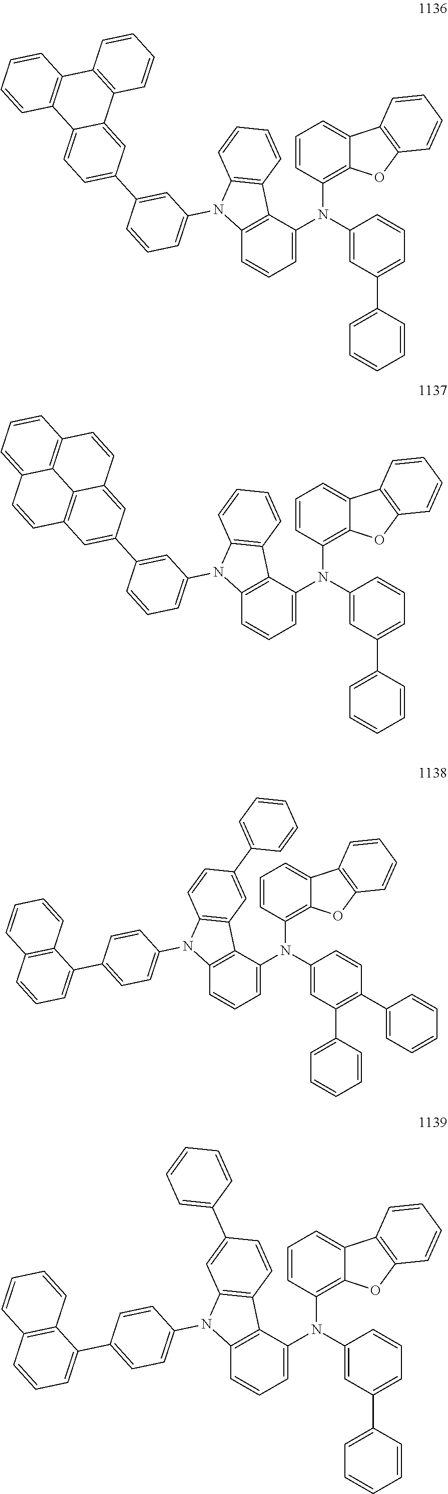 [SMILES: c1ccc(-c2cccc(N(c3cccc4c3oc3ccccc34)c3cccc4c3c3ccccc3n4-c3cccc(-c4ccc5c6ccccc6c6ccccc6c5c4)c3)c2)cc1 ; c1ccc(-c2cccc(N(c3cccc4c3oc3ccccc34)c3cccc4c3c3ccccc3n4-c3cccc(-c4cc5ccc6cccc7ccc(c4)c5c67)c3)c2)cc1 ; c1ccc(-c2ccc3c(c2)c2c(N(c4ccc(-c5ccccc5)c(-c5ccccc5)c4)c4cccc5c4oc4ccccc45)cccc2n3-c2ccc(-c3cccc4ccccc34)cc2)cc1 ; c1ccc(-c2cccc(N(c3cccc4c3oc3ccccc34)c3cccc4c3c3ccc(-c5ccccc5)cc3n4-c3ccc(-c4cccc5ccccc45)cc3)c2)cc1] 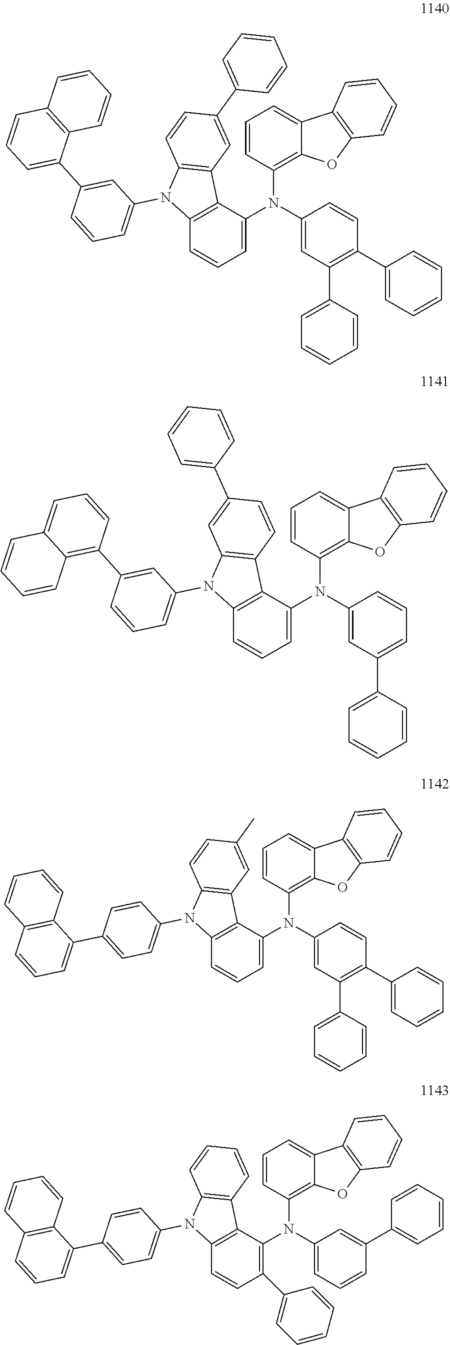 [SMILES: c1ccc(-c2ccc3c(c2)c2c(N(c4ccc(-c5ccccc5)c(-c5ccccc5)c4)c4cccc5c4oc4ccccc45)cccc2n3-c2cccc(-c3cccc4ccccc34)c2)cc1 ; c1ccc(-c2cccc(N(c3cccc4c3oc3ccccc34)c3cccc4c3c3ccc(-c5ccccc5)cc3n4-c3cccc(-c4cccc5ccccc45)c3)c2)cc1 ; Cc1ccc2c(c1)c1c(N(c3ccc(-c4ccccc4)c(-c4ccccc4)c3)c3cccc4c3oc3ccccc34)cccc1n2-c1ccc(-c2cccc3ccccc23)cc1 ; c1ccc(-c2cccc(N(c3cccc4c3oc3ccccc34)c3c(-c4ccccc4)ccc4c3c3ccccc3n4-c3ccc(-c4cccc5ccccc45)cc3)c2)cc1] 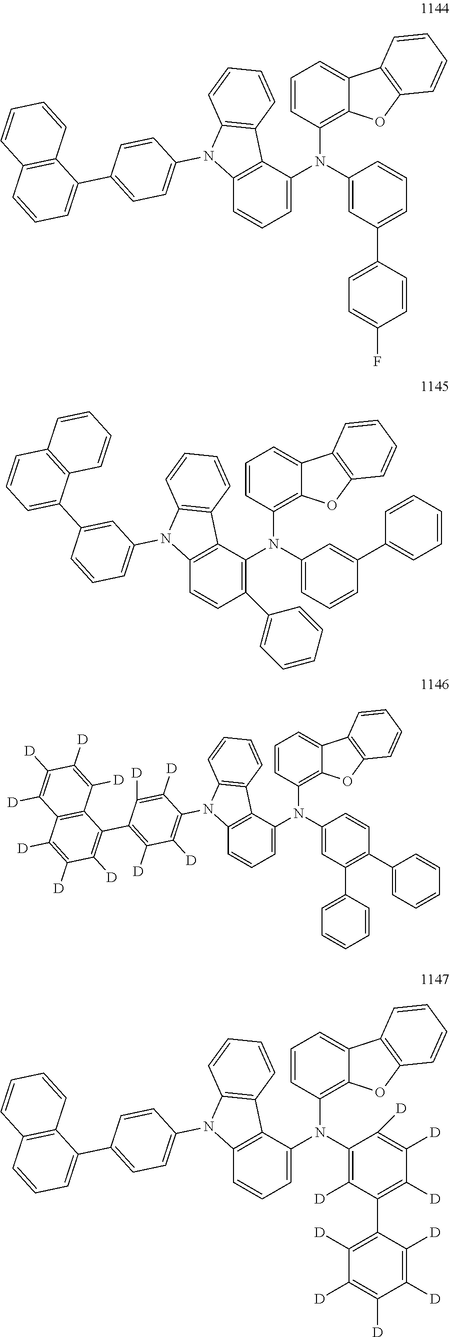 [SMILES: Fc1ccc(-c2cccc(N(c3cccc4c3oc3ccccc34)c3cccc4c3c3ccccc3n4-c3ccc(-c4cccc5ccccc45)cc3)c2)cc1 ; c1ccc(-c2cccc(N(c3cccc4c3oc3ccccc34)c3c(-c4ccccc4)ccc4c3c3ccccc3n4-c3cccc(-c4cccc5ccccc45)c3)c2)cc1 ; [2H]c1c([2H])c(-n2c3ccccc3c3c(N(c4ccc(-c5ccccc5)c(-c5ccccc5)c4)c4cccc5c4oc4ccccc45)cccc32)c([2H])c([2H])c1-c1c([2H])c([2H])c([2H])c2c([2H])c([2H])c([2H])c([2H])c12 ; [2H]c1c([2H])c([2H])c(-c2c([2H])c([2H])c([2H])c(N(c3cccc4c3oc3ccccc34)c3cccc4c3c3ccccc3n4-c3ccc(-c4cccc5ccccc45)cc3)c2[2H])c([2H])c1[2H]] 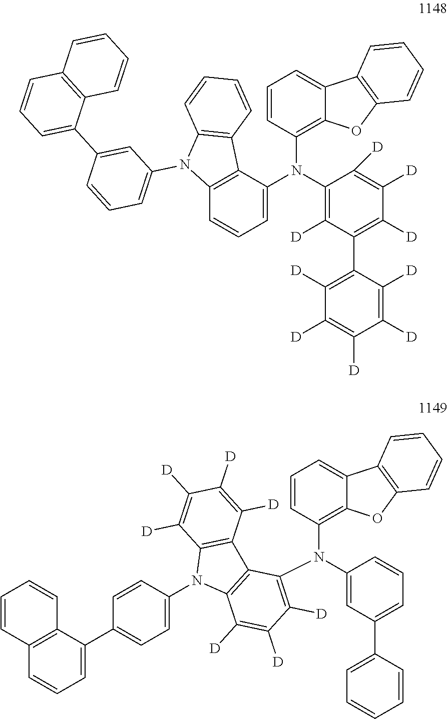 [SMILES: [2H]c1c([2H])c([2H])c(-c2c([2H])c([2H])c([2H])c(N(c3cccc4c3oc3ccccc34)c3cccc4c3c3ccccc3n4-c3cccc(-c4cccc5ccccc45)c3)c2[2H])c([2H])c1[2H] ; [2H]c1c([2H])c([2H])c2c(c1[2H])c1c(N(c3cccc(-c4ccccc4)c3)c3cccc4c3oc3ccccc34)c([2H])c([2H])c([2H])c1n2-c1ccc(-c2cccc3ccccc23)cc1]
in Compound Group 1, D is a deuterium atom.

20. A display device comprising:
a base layer;
a circuit layer on the base layer; and
a display element layer on the circuit layer and comprising a light emitting element,
the light emitting element comprising:
a first electrode;
a hole transport region on the first electrode;
an emission layer on the hole transport region;
an electron transport region on the emission layer; and
a second electrode on the electron transport region,
the hole transport region comprising an amine compound represented by Formula 1: 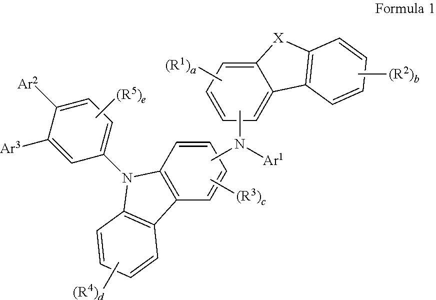
in Formula 1,
X is O or S,
Ar¹ is a substituted or unsubstituted aryl group of 6 to 30 ring-forming carbon atoms, or a substituted or unsubstituted heteroaryl group of 2 to 30 ring-forming carbon atoms,
Ar² and Ar³ are each independently a hydrogen atom, a deuterium atom, or a substituted or unsubstituted aryl group of 6 to 30 ring-forming carbon atoms, where at least one selected from among Ar² and Ar³ is a substituted or unsubstituted aryl group of 10 to 30 ring-forming carbon atoms,
R¹ to R⁵ are each independently a hydrogen atom, a deuterium atom, a halogen atom, a substituted or unsubstituted alkyl group of 1 to 15 carbon atoms, a substituted or unsubstituted aryl group of 6 to 30 ring-forming carbon atoms, or a substituted or unsubstituted heteroaryl group of 2 to 30 ring-forming carbon atoms,
“a”, “c” and “e” are each independently an integer of 0 to 3,
“b” and “d” are each independently an integer of 0 to 4,
when
a nitrogen atom of the amine compound is connected with position 2 or position 6 of a carbazole moiety, and is connected with position 3 or position 6 of a dibenzothiophene moiety or a dibenzofuran moiety, or
the nitrogen atom of the amine compound is connected with position 4 or position 5 of the carbazole moiety, and is connected with position 3 or position 6 of the dibenzothiophene moiety or a dibenzofuran moiety, then
a+b≥1, and at least one selected from among R¹ and R² is not a hydrogen atom, and
when the nitrogen atom of the amine compound is connected with position 3 of the carbazole moiety, then R³ is a hydrogen atom or a deuterium atom, and
Ar¹ is 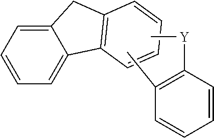
and Y is O or S, is excluded.
